(19) United States
(12) Patent Application Publication
(10) Pub. No.: US 2026/0061065 A1
(43) Pub. Date: Mar. 5, 2026

(54) ANTIBODY DRUG CONJUGATES

(71) Applicant: NOVARTIS AG, Basel (CH)

(72) Inventors: Matthew Terrence BURGER, Belmont, MA (US); Zhuoliang CHEN, Belmont, MA (US); Joseph Anthony D'ALESSIO, Cambridge, MA (US); Gregory John HOLLINGWORTH, Basel (CH); Claudia Judith KLINTER, Basel (CH); Jean-Baptiste Georges Armand LANGLOIS, Schlierbach (FR); Eric Andrew MCNEILL, Arlington, MA (US); Cornelia Anne MUNDT, Lörrach (DE); Katsumasa NAKAJIMA, Winchester, MA (US); Katharina MADÖRIN, Rünenberg (CH); Bing YU, Belmont, MA (US); Qiang ZHANG, East Brunswick, NJ (US)

(21) Appl. No.: 18/867,278

(22) PCT Filed: May 19, 2023
(86) PCT No.: PCT/IB2023/000277

§ 371 Date: Nov. 19, 2024

Related U.S. Application Data
(60) Provisional application No. 63/344,455, filed on May 20, 2022.

(51) Int. Cl. A61K 47/68

(52) U.S. Cl. CPC A61K 47/68031; A61K 47/6803

(57) ABSTRACT

The present disclosure relates to antibody-drug conjugates (ADCs) comprising an antibody or an antigen-binding fragment thereof covalently linked to two pharmaceutically active drugs through a dual linker. Linker-drug conjugates comprising the dual linker and the pharmaceutically active drugs are also disclosed. Such linkers are a convenient way of delivering two (e.g. two different or two of the same) drugs connected to a single antibody. Such linkers may be particularly useful in improving the solubility of antibody drug conjugates (ADCs) which comprise one or more hydrophobic drug compounds.

### RELATED APPLICATION

This application claims the benefit of the filing date, under 35 U.S.C. § 119(e), of U.S. Provisional Application No. 63/344,455, filed on May 20, 2022, the entire contents of which are incorporated here by reference.

### SEQUENCE LISTING

The instant application contains a Sequence Listing, which has been submitted electronically in ASCII XML format and is hereby incorporated by reference in its entirety. Said ASCII XML copy, created on Aug. 28, 2023, is named PAT059299-WO-PCT-ST26_SL.xml and is 568,832 bytes in size.

### FIELD OF THE INVENTION

The present disclosure relates to antibody-drug conjugates (ADCs) comprising an antibody or an antigen-binding fragment thereof covalently linked to two pharmaceutically active drugs through a dual linker. Linker-drug conjugates comprising the dual linker and the pharmaceutically active drugs are also disclosed. Such linkers are a convenient way of delivering two (e.g., two different or two of the same) drugs connected to a single antibody. Such linkers may be particularly useful in improving the solubility of antibody drug conjugates (ADCs) which comprise one or more hydrophobic drug compounds.

### BACKGROUND OF THE INVENTION

One aspect in the design of antibody drug conjugates (ADCs) is the design of the chemical linker, which links the drug moiety to the targeting moiety. Typically, an ADC uses a hydrophobic drug moiety, however when such drug moieties are used in combination with a relatively hydrophobic linker, solubility issues can arise which can affect the biocompatibility and pharmaceutical efficacy of the ADC.

Linker strategies have been reported to attempt to overcome these challenges, in particular the design of hydrophilic linkers incorporating polyethylene glycol (see R. P. Lyon, T. D. Bovee, S. O. Doronina, P. J. Burke, J. H. Hunter, H. D. Neff-LaFord, M. Jonas, M. E. Anderson, J. R. Setter, P. D. Senter, Nat. Biotechnol., 2015, 33, 733-735, and WO2015057699), linkers incorporating sulfonates (R. Y. Zhao, S. D. Wilhelm, C. Audette, G. Jones, B. A. Leece, A. C. Lazar, V. S. Goldmacher, R. Singh, Y. Kovtun, W. C. Widdison, J. M. Lambert, R. V. J. Chari, J. Med. Chen., 2011, 54, 3606-3623) and linkers having a carbohydrate backbone (F. S. Ekholm, H. Pynnönen, A. Vilkman, V. Pitksnen, J. Helin, J. Saarinen, T. Satomaa, ChemMedChem., 2016, 11(22):2501-2505).

There remains a need for antibody drug conjugate formats that allow for the targeted delivery of pharmaceutically active drugs, particularly hydrophobic drugs with improved pharmacokinetic and pharmacodynamic properties, in particular where the delivery of two pharmaceutically active drugs via a dual linker is desired.

### SUMMARY OF THE INVENTION

The invention provides compounds and conjugates whereby the delivery of two pharmaceutically active drugs via a dual linker is achieved, whilst simultaneously achieving a high level of solubility. The use of such linkers allows not only for the delivery of, if desired, two distinct drug molecules by a single monoclonal antibody, but also for the delivery of twice the number of drug moieties attached to the same antibody compared to antibody-drug conjugates known in the art (which can deliver 2, 4, 6 or 8 drugs per monoclonal antibody depending on the specific conjugation technology used). The compounds and conjugates described herein allow for the delivery of 4, 8, 12 or 16 drug molecules per monoclonal antibody.

Various embodiments of the invention are described herein.

According to a first aspect of the invention, there is hereby provided a compound of Formula (A-1), or a pharmaceutically acceptable salt thereof:

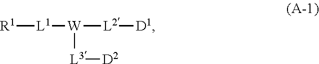

wherein:
R¹ is an attachment group;
L¹ is a bridging spacer;
W is a branching moiety;
L^{2′} and L^{3′} are each independently a linker; and
D¹ and D² are each independently a pharmaceutically active drug, optionally with the proviso that neither D¹ nor D² is a BH3 mimetic.

According to a second aspect of the invention, there is hereby provided a compound of Formula (B-1), or a pharmaceutically acceptable salt thereof:

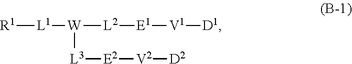

wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, an enzyme cleavage element or a hydrophilic moiety (e.g. E¹ and E² are each an enzyme cleavage element);
V¹ and V² are each independently i) a self-immolative group, or ii) an enzyme cleavage element (e.g. V¹ and V² are each independently a self-immolative group); and
D¹ and D² are each independently a pharmaceutically active drug, optionally with the proviso that neither D¹ nor D² is a BH3 mimetic.

According to a third aspect of the invention, there is hereby provided a compound of Formula (C-1), or a pharmaceutically acceptable salt thereof:

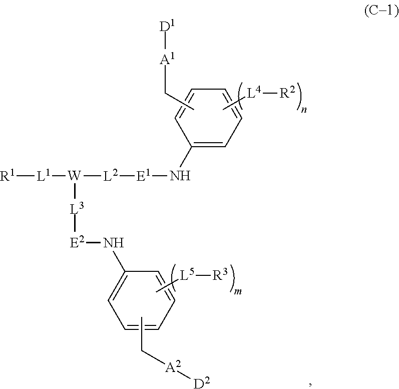

wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently a peptide group comprising 1 to 6 amino acids, wherein said peptide group is optionally substituted by a hydrophilic group;
A¹ and A² are each independently a bond, —OC(═O)—*,

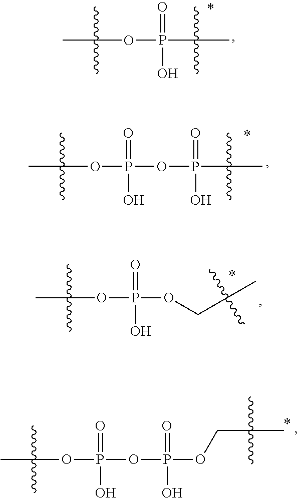 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆alkyl, and C₃-C₈cycloalkyl and the * of A¹ or A² indicates the point of attachment to D¹ or D²;
D¹ and D² are each independently a pharmaceutically active drug, optionally with the proviso that neither D¹ nor D² is a BH3 mimetic;
L⁴ and L⁵ are each independently a spacer moiety;
R² and R³ are each independently a hydrophilic group or an enzyme cleavage element (e.g. R² and R³ are each a hydrophilic group); and
m and n are each independently 0 or 1.

According to a fourth aspect of the invention, there is hereby provided a conjugate. In one embodiment of this aspect, the conjugate comprises an antibody or an antigen-binding fragment thereof covalently linked to two pharmaceutically active drugs through a dual linker, and wherein the dual linker has one attachment point connected to the antibody and two attachment points to the two pharmaceutically active drugs, with the proviso that neither pharmaceutically active drug is a BH3 mimetic.

In another embodiment of this aspect, there is hereby provided a conjugate of formula (A-2):

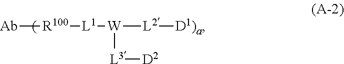

wherein:
Ab is an antibody or fragment thereof;
R¹⁰⁰ is a coupling group;
L¹ is a bridging spacer;
W is a branching moiety;
L^{2′} and L^{3′} are each independently a linker;
D¹ and D² are each independently a pharmaceutically active drug, optionally with the proviso that neither D¹ nor D² is a BH3 mimetic; and
a is an integer from 1 to 16.

According to a fifth aspect of the invention, there is hereby provided a conjugate of formula (B-2):

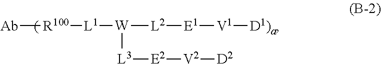

wherein:
Ab is an antibody or fragment thereof;
R¹⁰⁰ is a coupling group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, an enzyme cleavage element or a hydrophilic moiety (e.g. E¹ and E² are each an enzyme cleavage element);
V¹ and V² are each independently i) a self-immolative group, or ii) an enzyme cleavage element (e.g. V¹ and V² are each independently a self-immolative group);
D¹ and D² are each independently a pharmaceutically active drug, optionally with the proviso that neither D¹ nor D² is a BH3 mimetic; and
a is an integer from 1 to 16.

According to a sixth aspect of the invention, there is hereby provided a conjugate of formula (C-2):

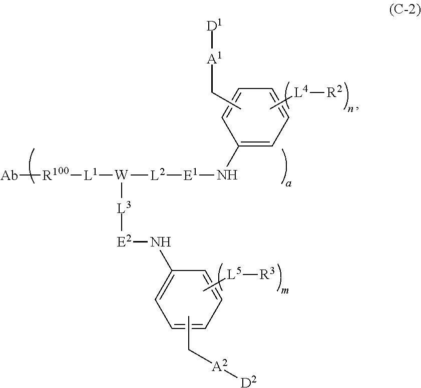

wherein:
Ab is an antibody or antigen-binding fragment thereof;
R¹⁰⁰ is a coupling group;
L¹ is a bridging spacer;
W is N or CR^{w}, wherein R^{w} is H or C₁-C₆alkyl (e.g. R^{w} is H); L² and L³ are each independently a connecting spacer;
E¹ and E² are each independently a peptide group comprising 1 to 6 amino acids, wherein said peptide group is optionally substituted by a hydrophilic group;
a is an integer from 1 to 16;

A¹ and A² are each independently a bond, —OC(═O)—*,

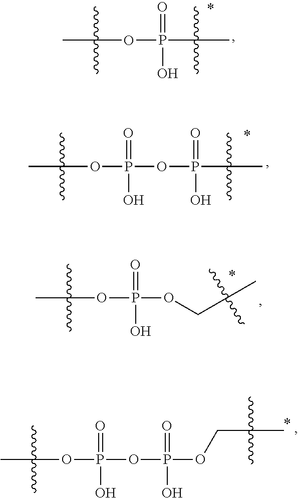 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆alkyl, and C₃-C₈cycloalkyl and the * of A¹ or A² indicates the point of attachment to D¹ or D²;
D¹ and D² are each independently a pharmaceutically active drug, optionally with the proviso that neither D¹ nor D² is a BH3 mimetic;
L⁴ and L⁵ are each independently a spacer moiety;
R² and R³ are each independently a hydrophilic group or an enzyme cleavage element (e.g. R² and R³ are each a hydrophilic group); and
m and n are each independently 0 or 1.

According to a seventh aspect of the invention, there is hereby provided a pharmaceutical composition comprising the conjugate of the fourth, fifth or sixth aspect of the invention, and a pharmaceutically acceptable carrier.

According to an eighth aspect of the invention, there is hereby provided a method of treating a subject having or suspected of having a cancer, comprising administering to the subject a therapeutically effective amount of the conjugate of the fourth, fifth or sixth aspect of the invention or the pharmaceutical composition of the pharmaceutical composition of the seventh aspect of the invention.

According to a ninth aspect of the invention, there is hereby provided a method of reducing or inhibiting the growth of a tumor in a subject, comprising administering to the subject a therapeutically effective amount of the conjugate of the fourth, fifth or sixth aspect of the invention or the pharmaceutical composition of the pharmaceutical composition of the seventh aspect of the invention.

According to a tenth aspect of the invention, there is hereby provided a method of reducing or inhibiting a hematological cancer in a subject, comprising administering to the subject a therapeutically effective amount of the conjugate of the fourth, fifth or sixth aspect of the invention or the pharmaceutical composition of the pharmaceutical composition of the seventh aspect of the invention.

According to an eleventh aspect of the invention, there is hereby provided a method of reducing or slowing the expansion of a cancer cell population in a subject, comprising administering to the subject a therapeutically effective amount of the conjugate of the fourth, fifth or sixth aspect of the invention or the pharmaceutical composition of the pharmaceutical composition of the seventh aspect of the invention.

According to a twelfth aspect of the invention, there is hereby provided a use of an conjugate of the fourth, fifth or sixth aspect of the invention, or the pharmaceutical composition of the seventh aspect of the invention, for the manufacture of a medicament for (i) treating a subject having or suspected of having a cancer, (ii) reducing or inhibiting the growth of a tumor in a subject, (iii) reducing or inhibiting a hematological cancer in a subject, or (iv) reducing or slowing the expansion of a cancer cell population in a subject.

According to a thirteenth aspect of the invention, there is hereby provided an antibody-drug conjugate of the fourth, fifth or sixth aspect of the invention, or the pharmaceutical composition of the seventh aspect of the invention for use in (i) treating a subject having or suspected of having a cancer, (ii) reducing or inhibiting the growth of a tumor in a subject, (iii) reducing or inhibiting a hematological cancer in a subject, or (iv) reducing or slowing the expansion of a cancer cell population in a subject.

### BRIEF DESCRIPTION OF THE FIGURES

FIG. 1. The dose response curves of Trastuzumab-Example 8 and Trastuzumab-Example 7 against representative cancer cell lines.

FIG. 2. The dose response curve of Trastuzumab-Example 5 against representative cancer cell lines.

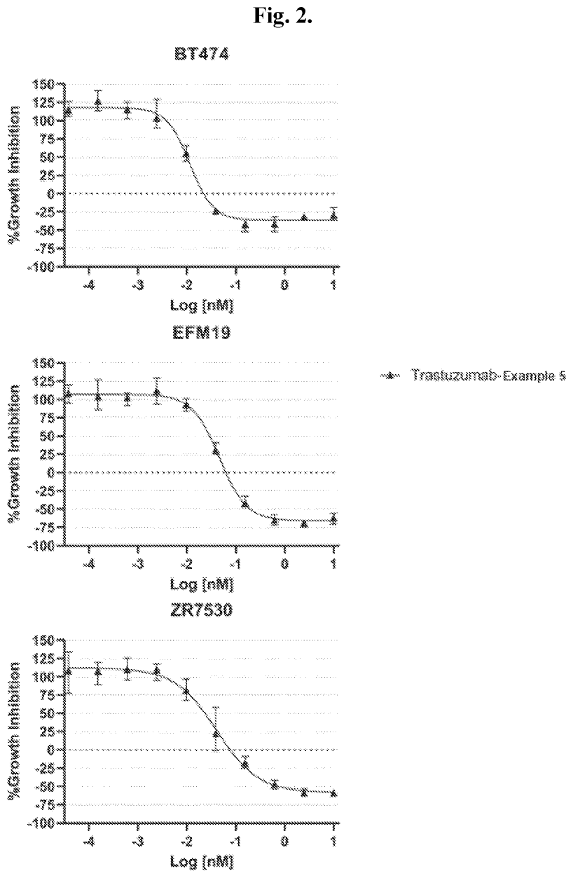

FIG. 3. The dose response curve of Trastuzumab-Example 6 against representative cancer cell lines.

### DETAILED DESCRIPTION

The disclosed compositions and methods may be understood more readily by reference to the following detailed description taken in connection with the accompanying figures, which form a part of this disclosure.

Throughout this text, the descriptions refer to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control.

Unless the context of a description indicates otherwise, e.g., in the absence of symbols indicating specific point(s) of connectivity, when a structure or fragment of a structure is drawn, it may be used on its own or attached to other components of an ADC, and it may do so with any orientation, e.g., with the antibody attached at any suitable attachment point to a chemical moiety such as a linker-drug. Where indicated, however, components of an ADC are attached in the orientation shown in a given formula. For example, if Formula (1) is described as

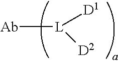

and the group

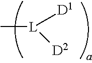

is described as

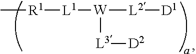

then the elaborated structure of Formula (1) is

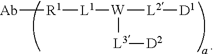

It is neither

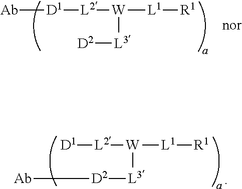

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

As used throughout this application, antibody drug conjugates can be identified using a naming convention in the general format of “target antigen/antibody-payload-dual linker-payload”. For example only, if an antibody drug conjugate is referred to as “Target X-P1-L1-P2”, such a conjugate would comprise an antibody that binds Target X, a dual linker designated as L1, and two payloads designated as P1 and P2, respectively. Alternatively, if an antibody drug conjugate is referred to as “anti-Target X-P1-L1-P2”, such a conjugate would comprise an antibody that binds Target X, a dual linker designated as L1, and two payloads designated as P1 and P2, respectively. In another alternative, if an antibody drug conjugate is referred to as “AbX-P1-L1-P2”, such a conjugate would comprise the antibody designated as AbX, a dual linker designated as L1, and two payloads designated as P1 and P2, respectively. A control antibody drug conjugate comprising a non-specific, isotype control antibody may be referenced as “isotype control IgG1-P1-L1-P2” or “IgG1-P1-L1-P2”.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen, carbon, nitrogen, oxygen, fluorine, and chlorine, such as ³H, ¹¹C, ¹³C, ¹⁴C, ¹⁵N, ¹⁸F, and ³⁶Cl. Accordingly, it should be understood that the present disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds can generally be prepared by conventional techniques known to those skilled in the art, e.g., using an appropriate isotopically-labeled reagents in place of the non-labeled reagent previously employed.

According to one aspect of the invention there is hereby disclosed a compound of Formula (A-1), or a pharmaceutically acceptable salt thereof:

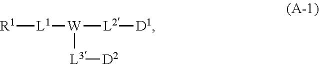

wherein:
R¹ is an attachment group;
L¹ is a bridging spacer;
W is a branching moiety;
L^{2′} and L^{3′} are each independently a linker; and
D¹ and D² are each independently a pharmaceutically active drug, optionally with the proviso that neither D¹ nor D² is a BH3 mimetic.

In an embodiment, each of L^{2′} and L^{3′} comprises a cleavable group, optionally at least one cleavable group comprises a phosphate group, a pyrophosphate group, a peptide group, and/or a self-immolative group (e.g. a peptide group and a self-immolative group).

In an embodiment, each of L^{2′} and L^{3′} comprises a peptide group and a self-immolative group.

In an embodiment, each of L^{2′} and L^{3′} comprises a cleavable group, optionally at least one cleavable group comprises a phosphate group, a pyrophosphate group, a sugar (e.g. glucuronamide) group, a peptide group, and/or a self-immolative group (e.g. a peptide group and a self-immolative group).

In an embodiment, the compound is a compound of formula (B-1):

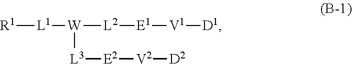

wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, a peptide group comprising 1 to 6 amino acids, wherein said peptide group is optionally substituted by a hydrophilic moiety;
V¹ and V² are independently comprise i) a self-immolative group, ii) an enzyme cleavage element (e.g. V¹ and V² are each independently a self-immolative group) or iiii) a self-immolative group and an enzyme cleavage element; and
D¹ and D² are each independently a pharmaceutically active drug, optionally with the proviso that neither D¹ nor D² is a BH3 mimetic;
or a pharmaceutically acceptable salt thereof. In an embodiment, the compound is a compound of formula (B-1):

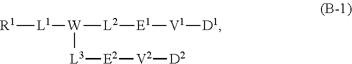

wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, a peptide group comprising 1 to 6 amino acids, wherein said peptide group is optionally substituted by a hydrophilic moiety;
V¹ and V² are each independently i) a self-immolative group, or ii) an enzyme cleavage element (e.g. V¹ and V² are each independently a self-immolative group); and
D¹ and D² are each independently a pharmaceutically active drug, optionally with the proviso that neither D¹ nor D² is a BH3 mimetic;
or a pharmaceutically acceptable salt thereof.

In an embodiment, W is N or CH.

In an embodiment, E¹ and E² are each an enzyme cleavage element.

In an embodiment, V¹ and V² are each independently a self-immolative group.

In an embodiment, (i) the cleavable linker comprises a phosphate group, a pyrophosphate group and/or a self-immolative group; (ii) the cleavable linker comprises a self-immolative group; (iii) the cleavable linker comprises a self-immolative group comprising —O—CH₂—, —NH—CH₂—, —OC(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium; or (iv) V¹ and/or V² each independently comprise a group comprising para-aminobenzyl-phosphate or para-aminobenzyl-pyrophosphate.

In an embodiment, (i) the cleavable linker comprises a phosphate group, a pyrophosphate group and/or a self-immolative group; (ii) the cleavable linker comprises a self-immolative group; or (iii) the cleavable linker comprises a self-immolative group comprising —O—CH₂—, —NH—CH₂—, OC(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.

In an embodiment, the compound is a compound of formula (C-1):

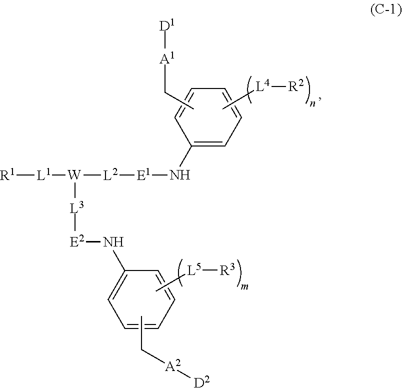

wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently a peptide group comprising 1 to 6 amino acids, wherein said peptide group is optionally substituted by a hydrophilic group;
A¹ and A² are each independently a bond, —OC(═O)—*,

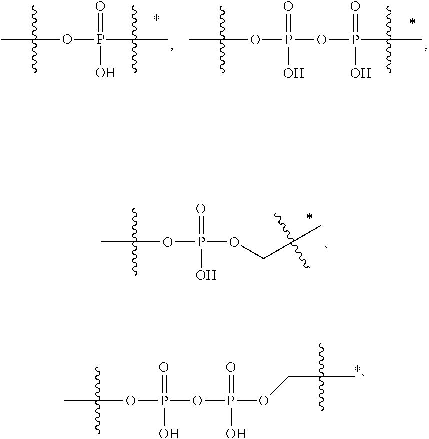 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆alkyl, and C₃-C₈cycloalkyl and the * of A¹ or A² indicates the point of attachment to D¹ or D²;
D¹ and D² are each independently a pharmaceutically active drug, optionally with the proviso that neither D¹ nor D² is a BH3 mimetic;
L⁴ and L⁵ are each independently a spacer moiety;
R² and R³ are each independently a hydrophilic group or an enzyme cleavage element (e.g. R² and R³ are each a hydrophilic group); and
m and n are each independently 0 or 1;
or a pharmaceutically acceptable salt thereof.

In an embodiment, W is N or CH.

In an embodiment, R² and R³ are each a hydrophilic group.

In an embodiment, the compound is a compound of formula (D1-1), (D2-1) or (D3-1):

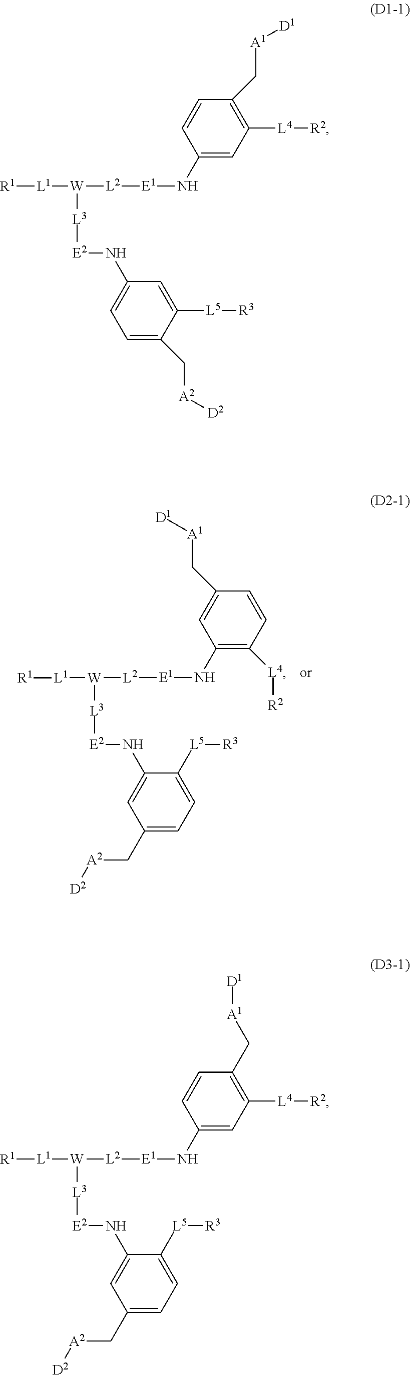

or a pharmaceutically acceptable salt thereof.

In an embodiment, Formula (D1-1), R² and R³ are each independently a hydrophilic group, wherein for Formula (D2-1), R² and R³ are each independently an enzyme cleavage element; and for Formula (D3-1), R² is a hydrophilic group and R³ is an enzyme cleavage element; or a pharmaceutically acceptable salt thereof.

In an embodiment, R¹ is selected from the group consisting of:

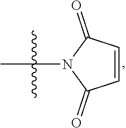 [SMILES: *N1C(=O)C=CC1=O]

—ONH₂, —NH₂,

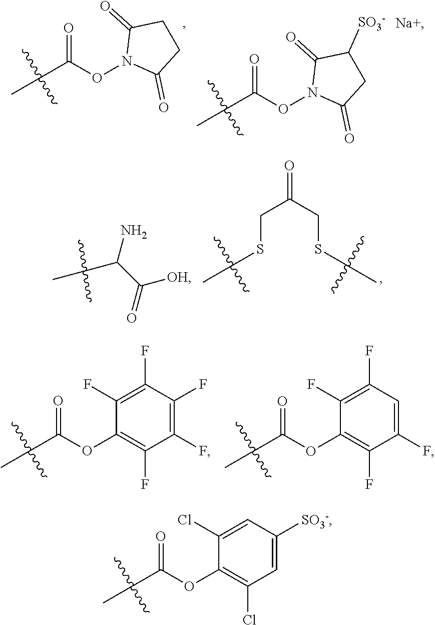

### —N₃,

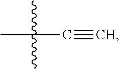 [SMILES: *C#C]

—SH, —SR¹¹, —SSR¹², —S(═O)₂(CH═CH₂), —(CH₂)₂S(═O)₂(CH═CH₂), —NHS(═O)₂(CH═CH₂), —NR¹¹C(═O)CH₂Br, e.g. —NHC(═O)CH₂Br, —NR¹¹C(═O)CH₂I, e.g. —NHC(═O)CH₂I,

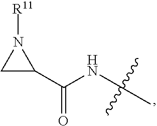

### —C(O)NHNH₂,

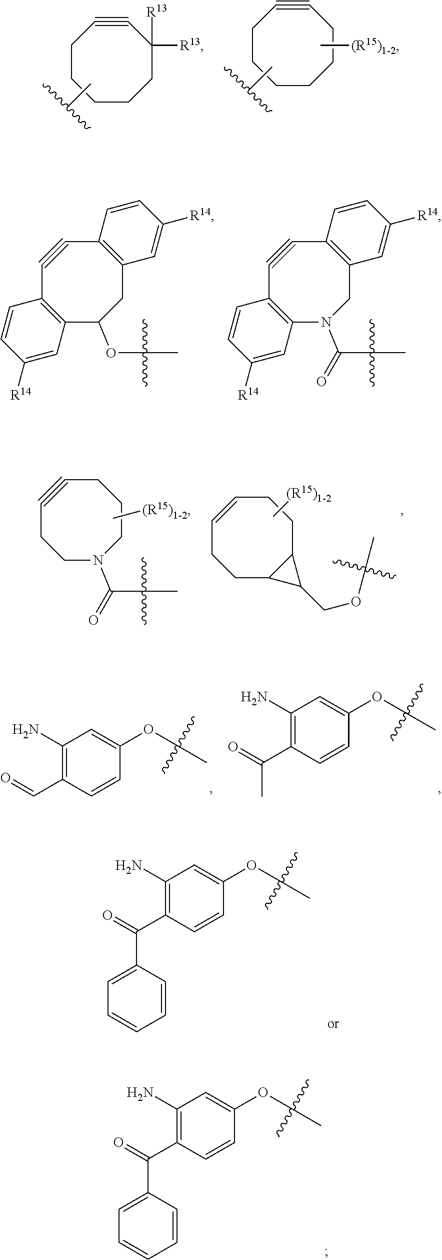

wherein:
each R¹¹ is independently selected from H and C₁-C₆alkyl;
each R¹² is 2-pyridyl or 4-pyridyl;
each R¹³ is independently selected from H, C₁-C₆alkyl, F, Cl, and —OH;
each R¹⁴ is independently selected from H, C₁-C₆alkyl, F, Cl, —NH₂, —OCH₃, —OCH₂CH₃, —N(CH₃)₂, —CN, —NO₂ and —OH; and
each R¹⁵ is independently selected from H, C₁-C₆alkyl, fluoro, benzyloxy substituted with —C(═O)OH, benzyl substituted with —C(═O)OH, C₁-C₄alkoxy substituted with —C(═O)OH and C₁-C₄alkyl substituted with —C(═O)OH.

In an embodiment, R¹ is selected from the group consisting of:

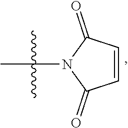 [SMILES: *N1C(=O)C=CC1=O]

### —ONH₂,

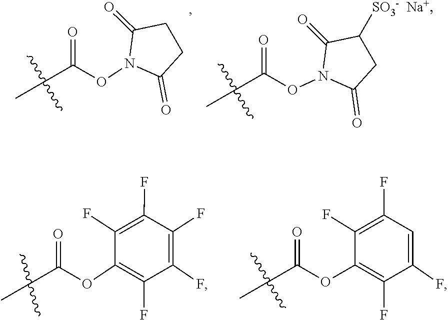

NHC(═O)CH₂Br and —NHC(═O)CH₂I.

In an embodiment, R¹ is

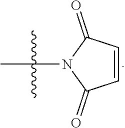 [SMILES: *N1C(=O)C=CC1=O]

In an embodiment, (1) L¹ comprises:

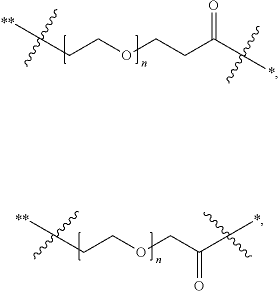

### or *—CH(OH)CH(OH)CH(OH)CH(OH)—**,

wherein each n is an integer from 1 to 12, wherein the * of L¹ indicates the point of direct or indirect (e.g. direct) attachment to W, and the ** of L¹ indicates the point of direct or indirect (e.g. direct) attachment to R¹;
(2) L¹ is

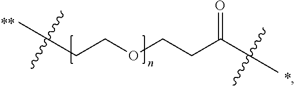

and n is an integer from 1 to 12 or n is 1 or n is 12, wherein the * of L¹ indicates the point of direct or indirect (e.g. direct) attachment to W, and the ** of L¹ indicates the point of direct or indirect (e.g. direct) attachment to R¹;
(3) L¹ is

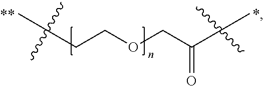

and n is an integer from 1 to 12, wherein the * of L¹ indicates the point of direct or indirect (e.g. direct) attachment to W, and the ** of L₁ indicates the point of direct or indirect (e.g. direct) attachment to R¹;
(4) L¹ comprises

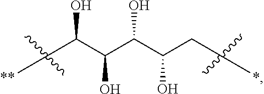 [SMILES: *C[C@H](O)[C@@H](O)[C@@H](O)[C@@H](*)O]

wherein the * of L¹ indicates the point of direct or indirect (e.g. direct) attachment to W, and the ** of L¹ indicates the point of direct or indirect (e.g. direct) attachment to R¹; or
(5) L¹ is a bridging spacer comprising:
—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R^{L1})₂(CH₂)ₘC(═O)NR^{L1}(CH₂)ₘNR^{L1}C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX¹(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R^{L1})₂—** or *—C(═O)(CH₂)ₘC(═O)NH(CH₂)ₘ—**, wherein the * of L¹ indicates the point of direct or indirect (e.g. direct) attachment to W, and the ** of L¹ indicates the point of direct or indirect (e.g. direct) attachment to R¹;
X₁ is

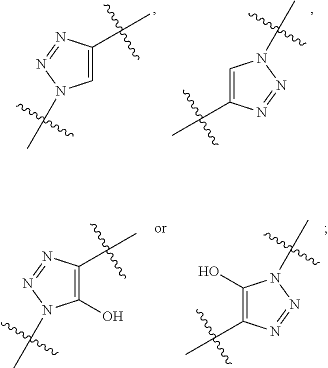 [SMILES: *c1cn(*)nn1 ; *c1cn(*)nn1 ; *c1nnn(*)c1O ; *c1nnn(*)c1O]

and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30; and
each R^{L1} is independently selected from H and C₁-C₆alkyl.
   In an embodiment, L¹ comprises a moiety represented by

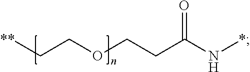

wherein n is an integer from 1 to 12 (e.g. 4 to 8, e.g. 4 or 8), wherein the * of L¹ indicates the point of direct or indirect (e.g. direct) attachment to W, and the ** of L¹ indicates the point of direct or indirect attachment to R¹.

In an embodiment, n is an integer from 4 to 8.

In an embodiment, n is 4 or 8.

In an embodiment, L¹ is represented by a formula

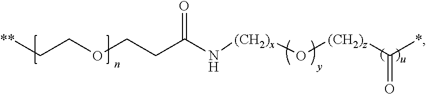

wherein
n is an integer from 1 to 12 (e.g. 4 to 8, e.g. 4 or 8);
x is an integer from 0 to 6 (e.g. 0 to 4, e.g. 0 to 2, e.g. 0 or 2);
y is 0 or 1;
z is an integer from 0 to 6 (e.g. 0 to 4, e.g. 0 to 2, e.g. 0 or 2);
u is 0 or 1; and
wherein the * of L¹ indicates the point of direct attachment to W, and the ** of L¹ indicates the point of direct attachment to R¹.

In an embodiment, n is an integer from 4 to 8.

In an embodiment, n is 4 or 8.

In an embodiment, x is an integer from 0 to 2.

In an embodiment, x is 0 or 2.

In an embodiment, z is an integer from 0 to 2.

In an embodiment, z is 0 or 2.

In an embodiment, L¹ is selected from the group consisting of:

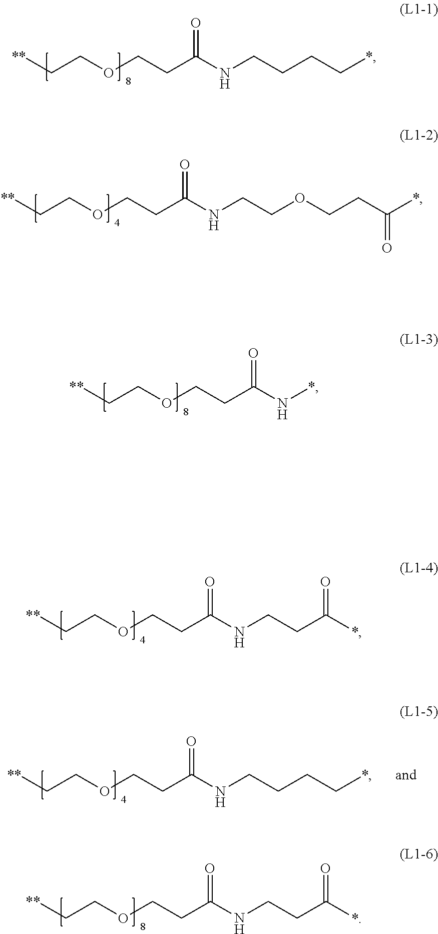

In an embodiment, L² and L³ are each independently a connecting spacer comprising a moiety represented by:

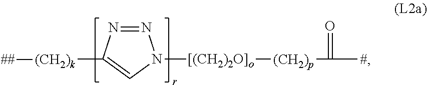

wherein
k is an integer from 0 to 6;
r is 0 or 1;
o is an integer from 0 to 12;
p is an integer from 0 to 6; and
   wherein the # of L² or L³ indicates the point of direct or indirect attachment to E¹ or E², respectively, and the ## of L² or L³ indicates the point of direct or indirect attachment to W.
   In an embodiment, L² and L³ are each independently a connecting spacer selected from a group consisting of

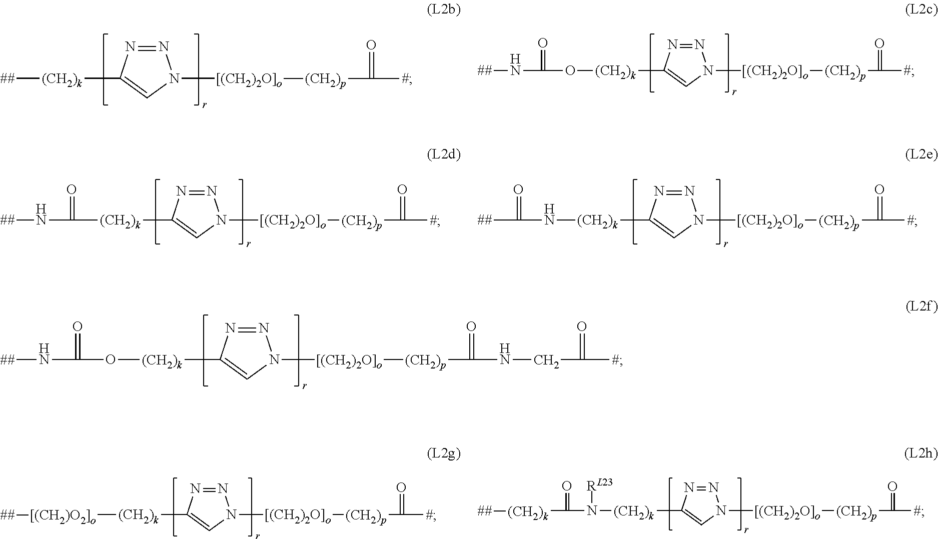

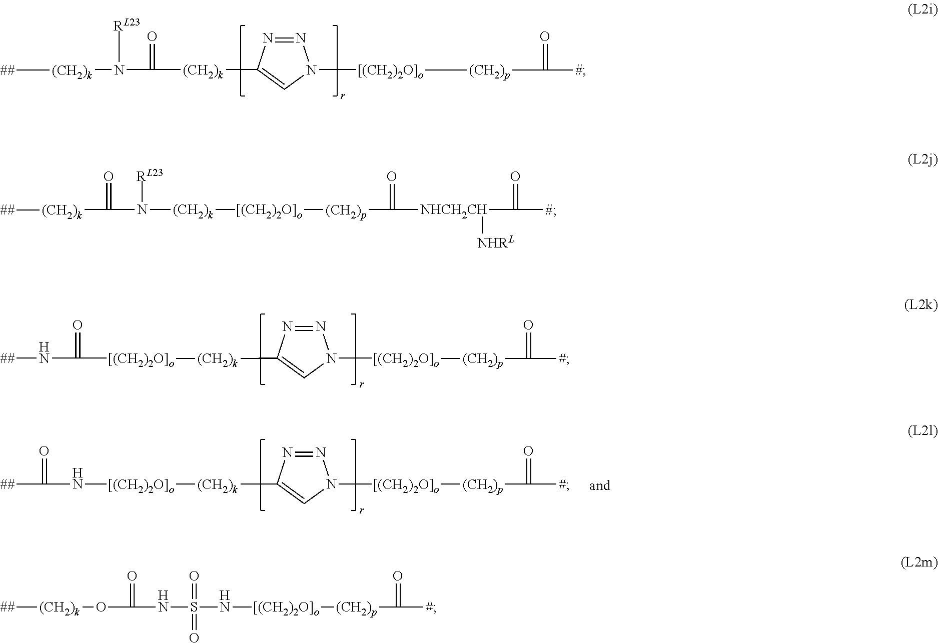

wherein
k, in each occurrence, is independently an integer from 0 to 4;
r, in each occurrence, is independently 0 or 1;
o, in each occurrence, is independently an integer from 0 to 10;
p, in each occurrence, is independently an integer from 0 to 4;
R^{L23} is hydrogen or C₁-C₆alkyl;
R^{L} is hydrogen or —C(O)—R^{H};
R^{H} is a hydrophilic group; and
the # of L² or L³ indicates the point of direct attachment to E¹ or E², respectively, and the ## of L²
or L³ indicates the point of direct attachment to W;
provided that when W is N, L² and L³ are not (L2c), (L2d), (L2f) or (L2k).

In an embodiment, L² and L³ are each independently a connecting spacer selected from a group consisting of:

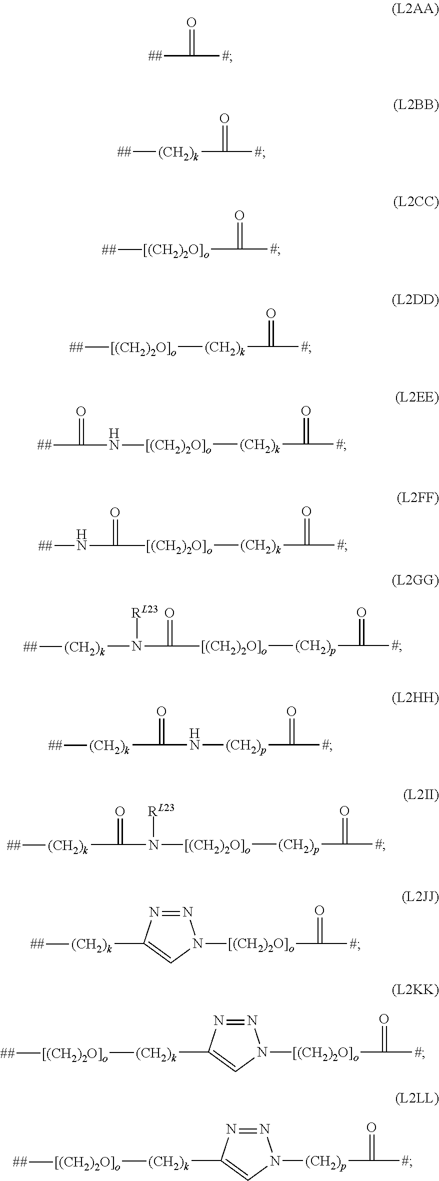

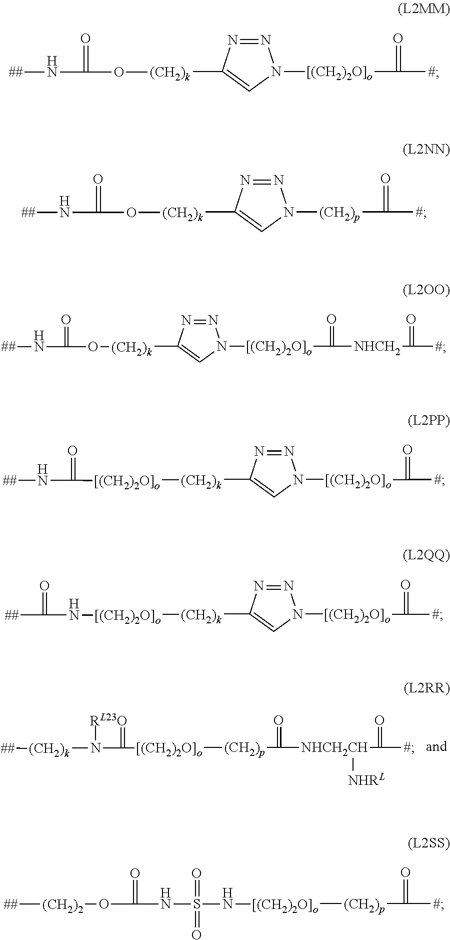

wherein
k, in each occurrence, is independently an integer from 1 to 3;
o, in each occurrence, is independently an integer from 1 to 9;
p, in each occurrence, is independently an integer from 1 to 3;
R^{L23} is hydrogen or C₁₋₃alkyl;
R^{L} is hydrogen or —C(O)—R^{H};
R^{H} is a hydrophilic group; and the # of L² or L³ indicates the point of direct attachment to E¹ or E², respectively, and the ## of L² or L³ indicates the point of direct attachment to W; provided that when W is N, L² and L³ are not (L2FF), (L2MM), (L2NN), (L2OO), or (L2PP).

In an embodiment, L² and L³, independently, are a connecting spacer selected from a group consisting of:

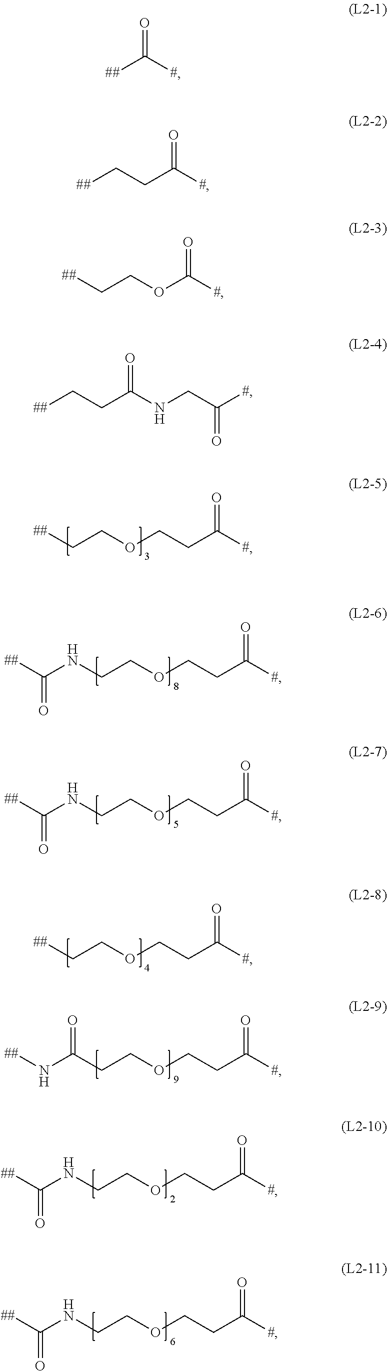

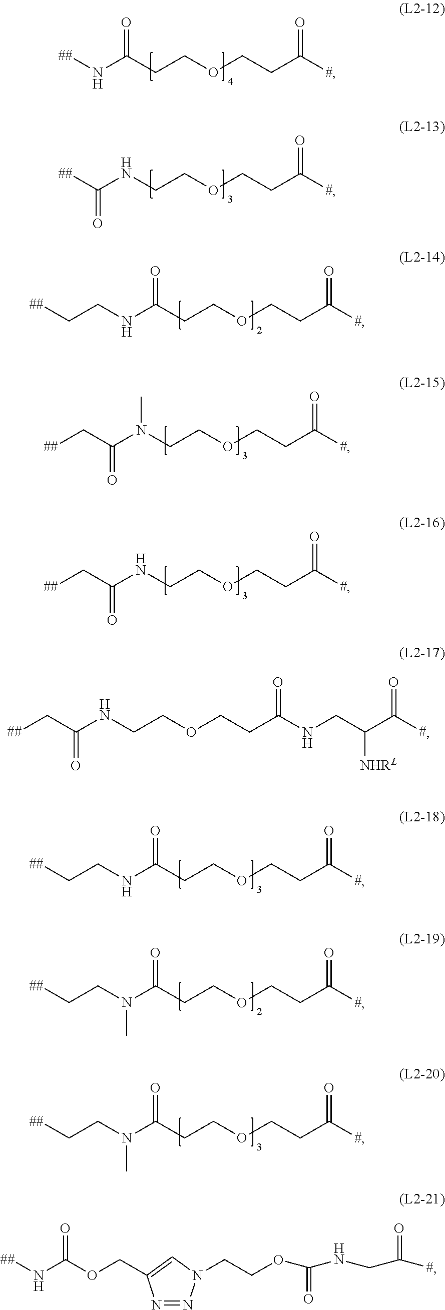

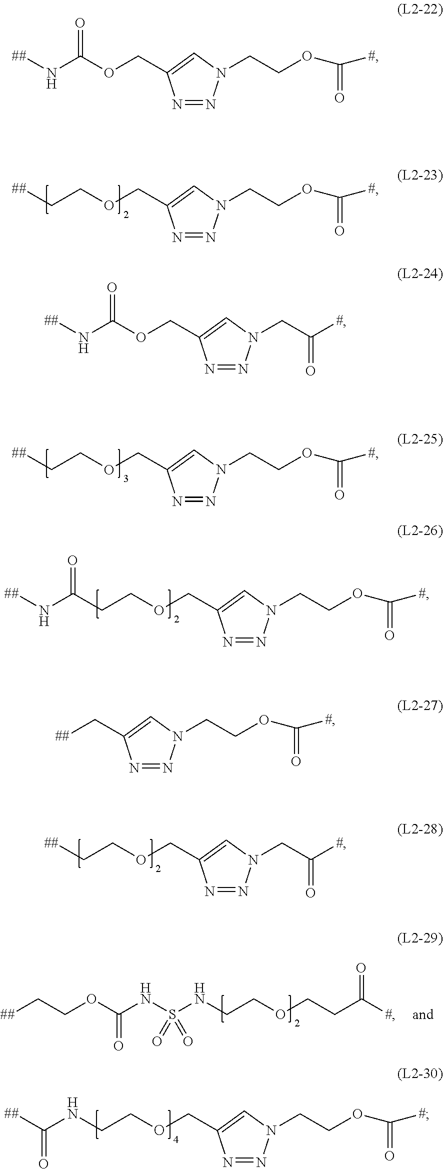

wherein the # of L² or L³ indicates the point of direct attachment to E¹ or E², respectively, the ## of L² or L³ indicates the point of direct attachment to W; R^{L} is hydrogen or —C(O)—R^{H}; and
R^{H} is

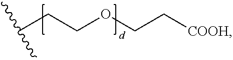

and d is an integer from 20 to 30 (e.g. 25).

In an embodiment, d is 25.

In an embodiment, each peptide group independently comprises 1 to 4, 1 to 3, or 1 to 2 amino acid residues.

In an embodiment, each peptide group independently comprises 2 amino acid residues.

In an embodiment, each amino acid residues is independently selected from glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), L-tyrosine (Tyr) and β-alanine (β-Ala).

In an embodiment, each peptide group is independently selected from Val-Cit, Phe-Lys, Val-Ala, Val-Lys, Leu-Cit, Cit-(β-Ala), Gly-Gly-Gly, Gly-Gly-Phe-Gly, and sulfo-Ala-Val-Ala.

In an embodiment, E¹ and/or E², independently, is/are each a peptide group selected from a group consisting of:

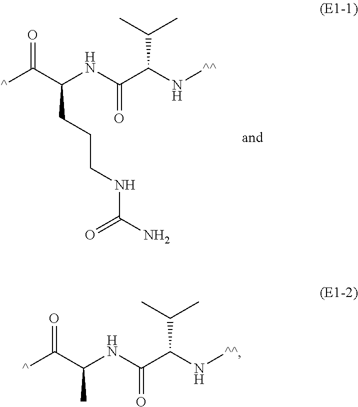

wherein \hat{ } of E1-1 or E1-2 indicates the point of direct attachment to V¹ or V² in Formula (B) or direct attachment to the —NH— group in Formula (C) and (D); and \hat{ }\hat{ } of E1-1 or E1-2 indicates the point of direct attachment to L² or L³, respectively (e.g. where E¹ and E² are each a peptide group independently selected from (E1-1) and (E1-2)).

In an embodiment, E¹ and E² are each a peptide group independently selected from (E1-1) and (E1-2).

In an embodiment, E¹ and/or E², independently, is/are each a peptide group represented by

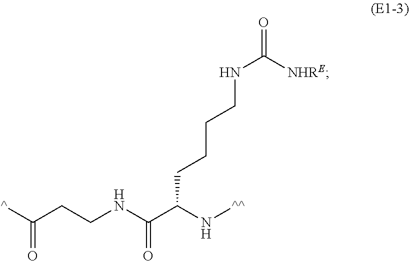

wherein R^{E} is a hydrophilic group R^{H}.

In an embodiment, the hydrophilic group R^{H} in (E1-3) is

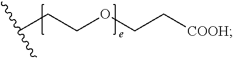

wherein e is an integer between 20 and 30 (e.g. 25).

In an embodiment, e is 25.

In an embodiment, E¹ and E² are each

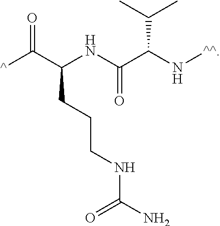

In an embodiment, A¹ and A² are independently selected from a bond and —OC(═O)—*, wherein * indicates the point of attachment to D¹ or D² respectively.

In an embodiment, A¹ and A² are independently selected from a bond,

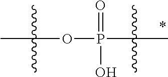 [SMILES: *OP(*)(=O)O]

OH and —OC(═O)—*, wherein * indicates the point of attachment to D¹ or D² respectively.

In an embodiment, A¹ and A² are each a bond.

In an embodiment, A¹ and A² are both-OC(═O)—*.

In an embodiment, one of A¹ and A² is OC(═O)—*, and the other of A¹ and A² is a bond.

In an embodiment, A¹ and A² independently are a bond or

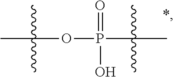 [SMILES: *OP(*)(=O)O]

wherein * indicates the point of attachment to D¹ or D².

In an embodiment, one of A¹ and A² is

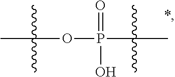 [SMILES: *OP(*)(=O)O]

and the other of A¹ and A² is a bond.

In an embodiment, A¹ and A² are both

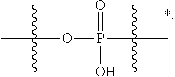 [SMILES: *OP(*)(=O)O]

In an embodiment, one of A¹ and A² is

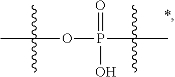 [SMILES: *OP(*)(=O)O]

and the other of A¹ and A² is OC(═O)—*.
In an embodiment, (i) A¹ and A² are —OC(═O)—*;
(ii) A¹ and A² are

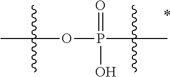 [SMILES: *OP(*)(=O)O]

(iii) A¹ is —OC(═O)—* and A² is a bond
(iv) A¹ is —OC(═O)—* and A² is

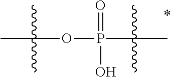 [SMILES: *OP(*)(=O)O]

(v) A¹ is a bond and A² is

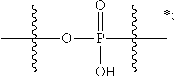 [SMILES: *OP(*)(=O)O]

or
(vi) A¹ is a bond and A² is —OC(═O)—*,
wherein * indicates the point of attachment to D¹ or D².

In an embodiment, i) L⁴ and L⁵ are each independently a spacer moiety having the structure

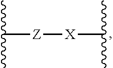

wherein:
Z is —O—, —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{L45}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is a bond, triazolyl, or —CH₂—triazolyl-,
wherein X is connected to R² or R³; or
(ii) L⁴ and L⁵, independently, are a spacer moiety having the structure

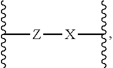

wherein:
Z is —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{b}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is —CH₂—triazolyl-C₁-C₄alkylene-OC(O)NHS(O)₂NH—, —C₄-C₆cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂—triazolyl-C₁-C₄alkylene-OC(O)NHS(O)₂NH—(CH₂CH₂O)ₙ—, —C₄-C₆cycloalkylene-OC(O)NHS(O)₂NH—(CH₂CH₂O)ₙ—, wherein each n independently is 1, 2, or 3,
wherein X is connected to R² or R³.

In an embodiment, Z is —O—, —CH₂NR^{L45}C(═O)—, —CH₂NR^{L45}C(═O)NH— or —CH₂O—; X is a bond, triazolyl, or —CH₂—triazolyl-; and R^{L45}, in each occurrence, is independently H or C₁-C₃alkyl.

In an embodiment, L⁴ and L⁵ are each independently a spacer moiety selected from a group consisting of

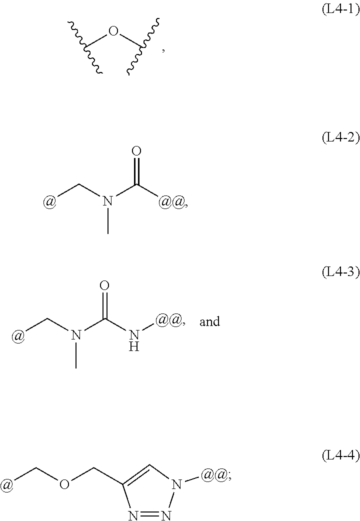

wherein the @ of L⁴ or L⁵ indicates the point of direct attachment to the phenyl group, and the @@ of L⁴ or L⁵ indicates the point of direct attachment to R² or R³.

In an embodiment, the hydrophilic groups represented by R² and R³ each independently comprises polyethylene glycol, polyalkylene glycol, a polyol, a polysarcosine, a sugar, an oligosaccharide, a polypeptide, C₂-C₆alkyl substituted with 1 to 3

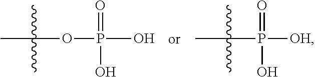 [SMILES: *OP(=O)(O)O ; *P(=O)(O)O]

or C₂—C₆alkyl substituted with 1 to 2 substituents independently selected from —OC(═O)NHS(O)₂NHCH₂CH₂OCH₃, —NHC(═O)C₁-C₄alkylene-P(O)(OCH₂CH₃)₂ and —COOH groups.

In an embodiment, R² or/and R³ independently is/are each selected from the group consisting of:

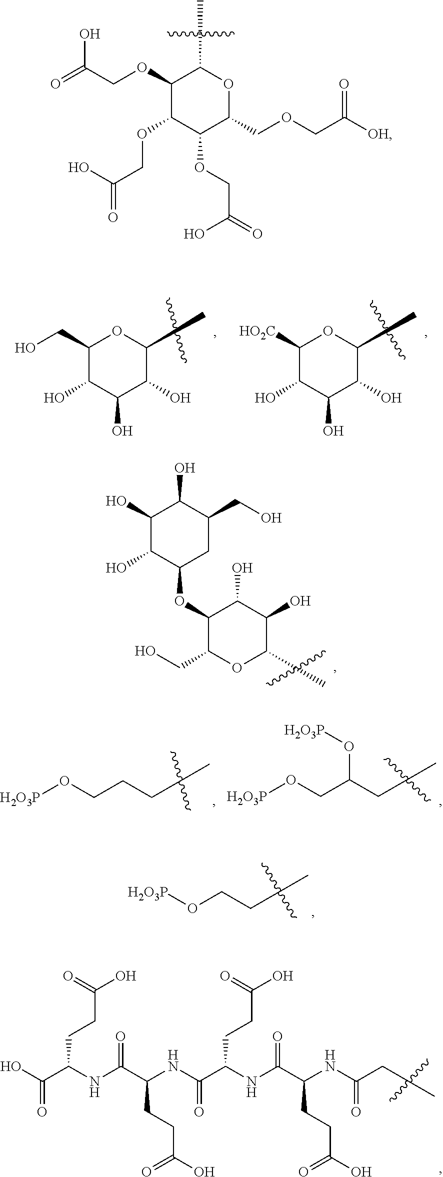

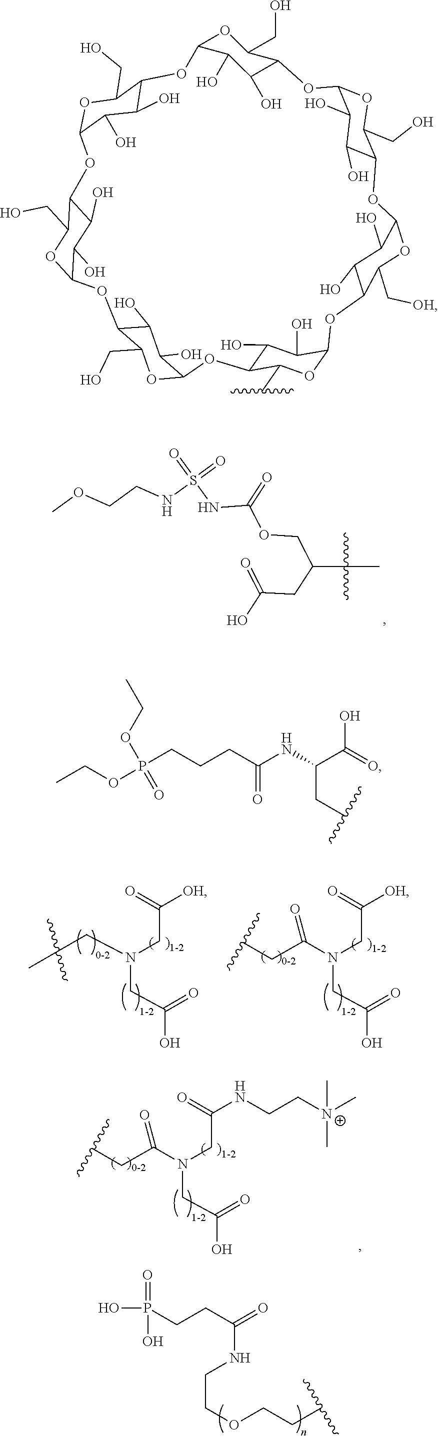

wherein n is an integer between 1 and 6,

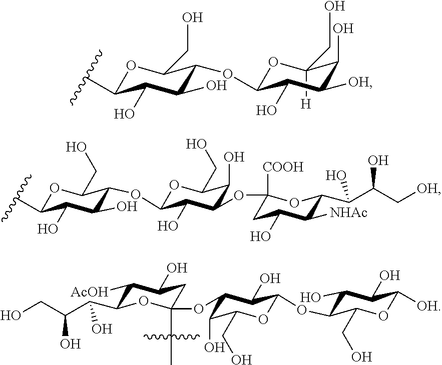

In an embodiment, R² or/and R³ independently comprises:
(i) a polysarcosine with the following moiety:

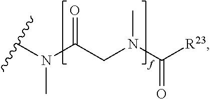

wherein
   f is an integer between 3 and 25; and
   R²³ is H, —CH₃ or —CH₂CH₂C(═O)OH;
(ii) a polyethylene glycol of formula:

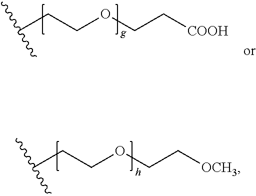

wherein
g and h are independently an integer between 2 and 30; or
(iii) a moiety represented by

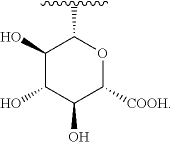 [SMILES: *[C@@H]1O[C@H](C(=O)O)[C@@H](O)[C@H](O)[C@H]1O]

In an embodiment, R² or/and R³, independently, is l are each selected from a group consisting of

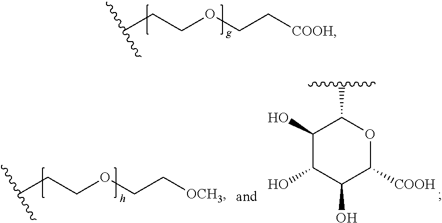

wherein
g and h are independently an integer between 20 and 30 (e.g. 23 or 24).

In an embodiment, R² or/and R³ comprises:

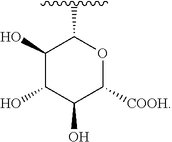 [SMILES: *[C@@H]1O[C@H](C(=O)O)[C@@H](O)[C@H](O)[C@H]1O]

In an embodiment, the hydrophilic group represented by R² or R³ each independently comprises:
(i) a polysarcosine with the following moiety:

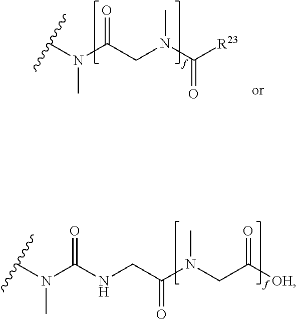

wherein f is an integer between 3 and 25;
and R²³ is H, —CH₃ or —CH₂CH₂C(═O)OH; or (ii) a polyethylene glycol of formula:

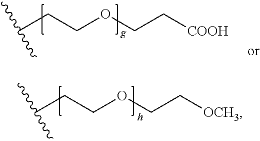

wherein g and h are independently an integer between 2 and 30.

In an embodiment, R² and R³ each independently comprises a polyethylene glycol of formula:

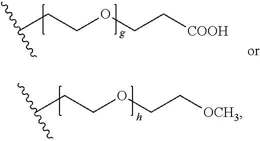

wherein g and h are independently an integer between 2 and 30.

In an embodiment, D¹ and D² are the same.

In an embodiment, D¹ and D² are different.

In an embodiment, D¹ and D² are each independently selected from a cytotoxic drug, a cytostatic drug and an immunosuppressive drug.

In an embodiment, D¹ and D² are each independently selected from an auristatin, a camptothecin, a duocarmycin, an etoposide, a maytansine, a maytansinoid, a taxane, a benzodiazepine or benzodiazepine containing drug (e.g., pyrrolo[1,4]-benzodiazepines (PBDs), indolinobenzodiazepines, and oxazolidinobenzodiazepines), and a vinca alkaloid.

In an embodiment, D¹ and D² are each independently selected from the group consisting of:

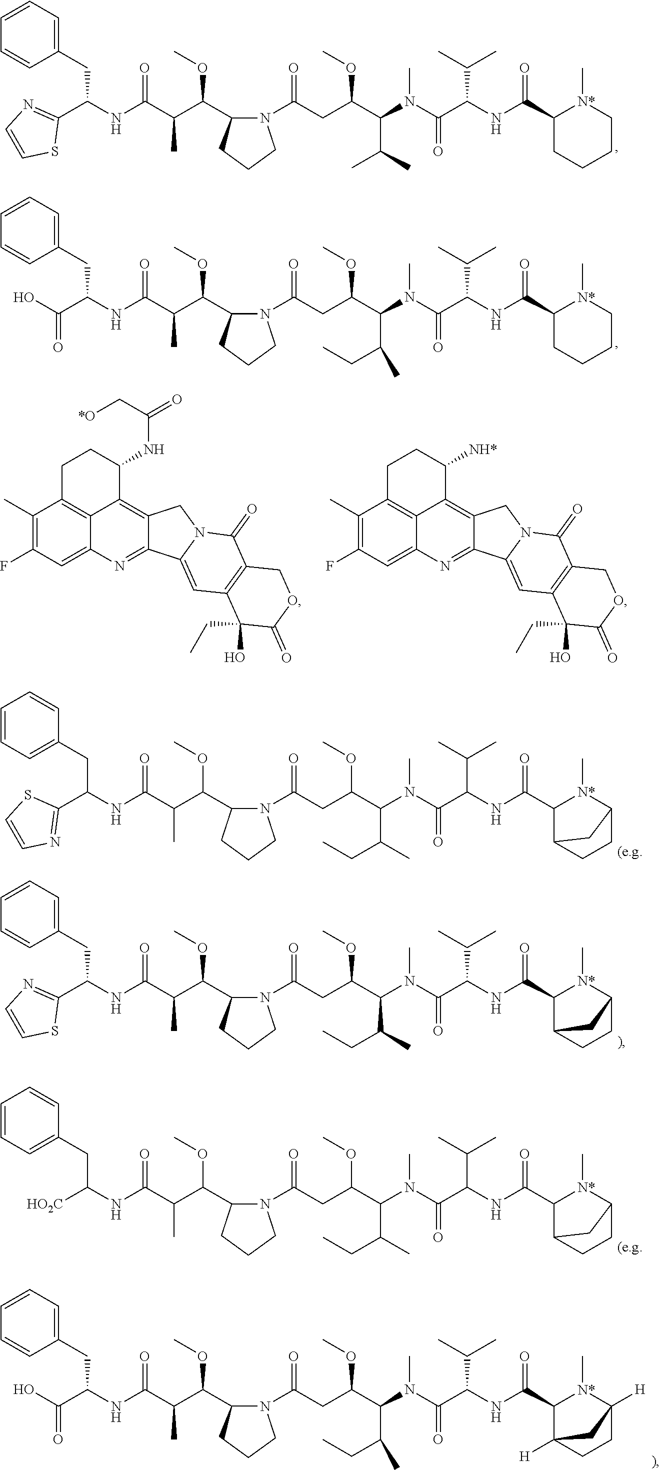

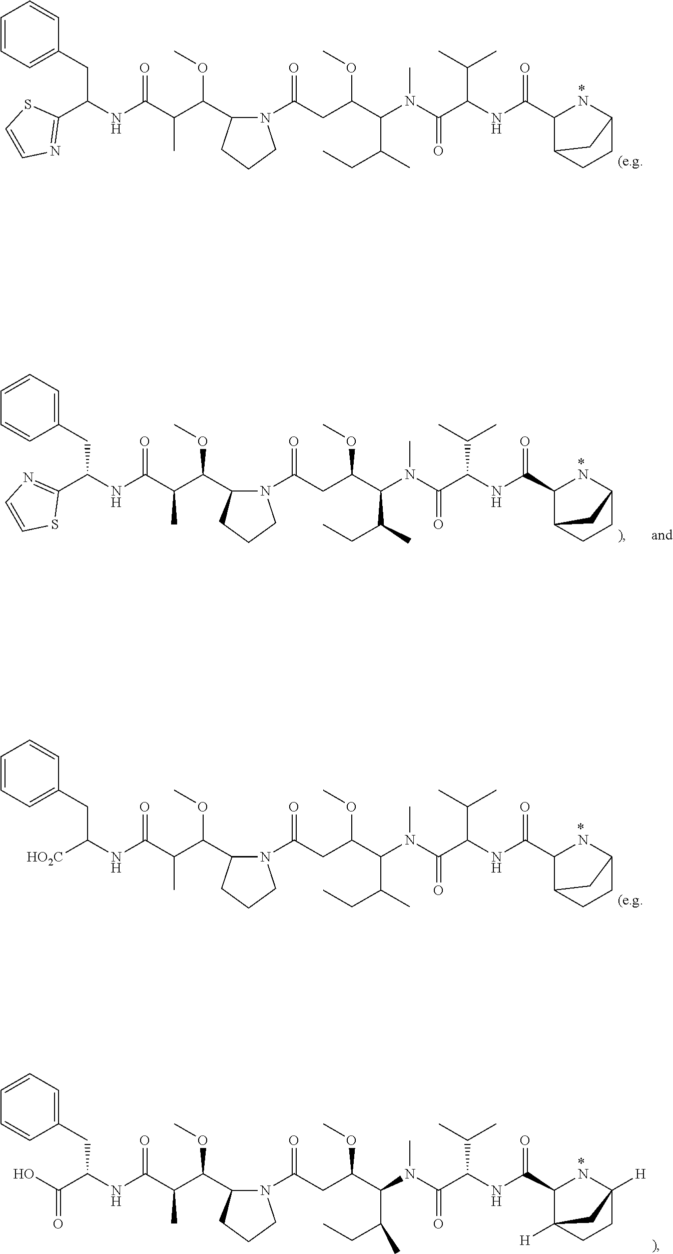

wherein * indicates the point of attachment to A¹ or A²; or a pharmaceutically acceptable salt thereof. It should be appreciated that where the N atom with a * in otherwise trivalent compounds such as

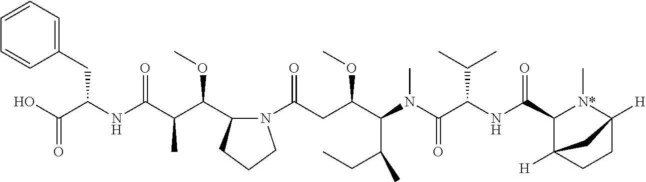

is attached to a linker, a cationic structure will result (cations are not shown for fragments of the molecules herein).
In an embodiment,

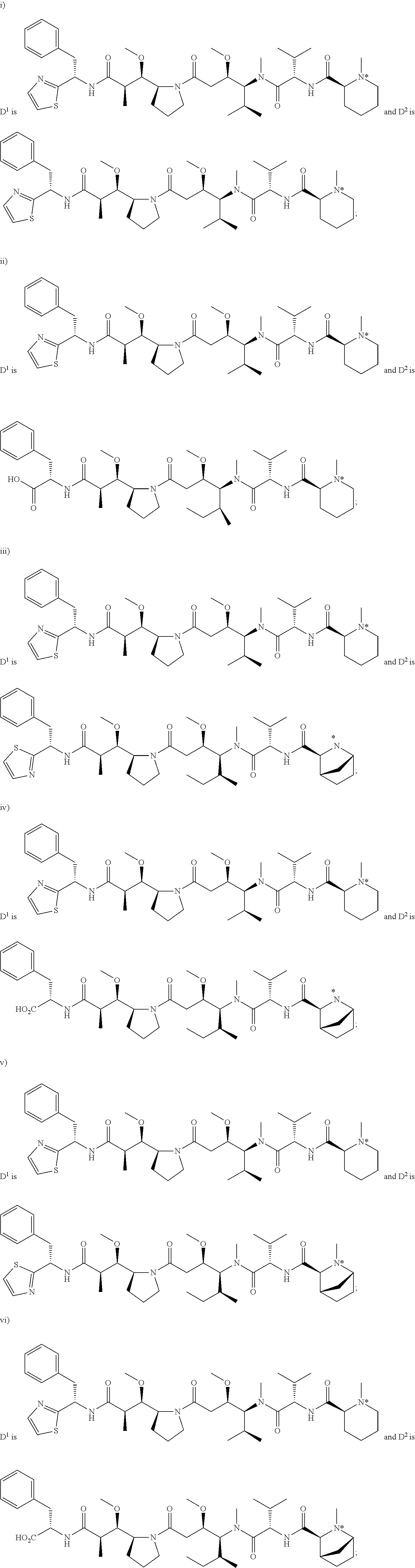

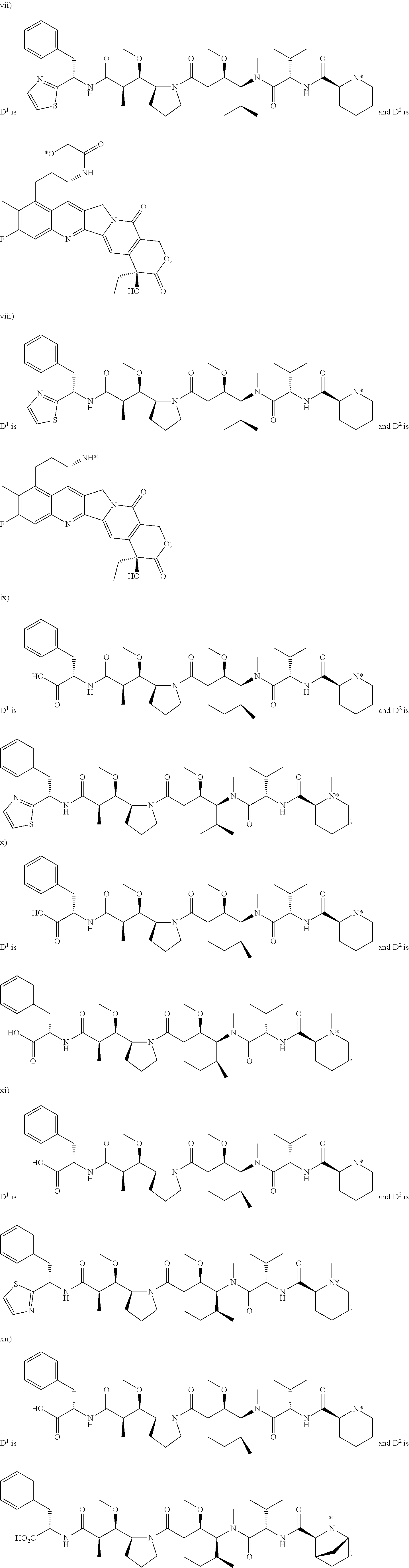

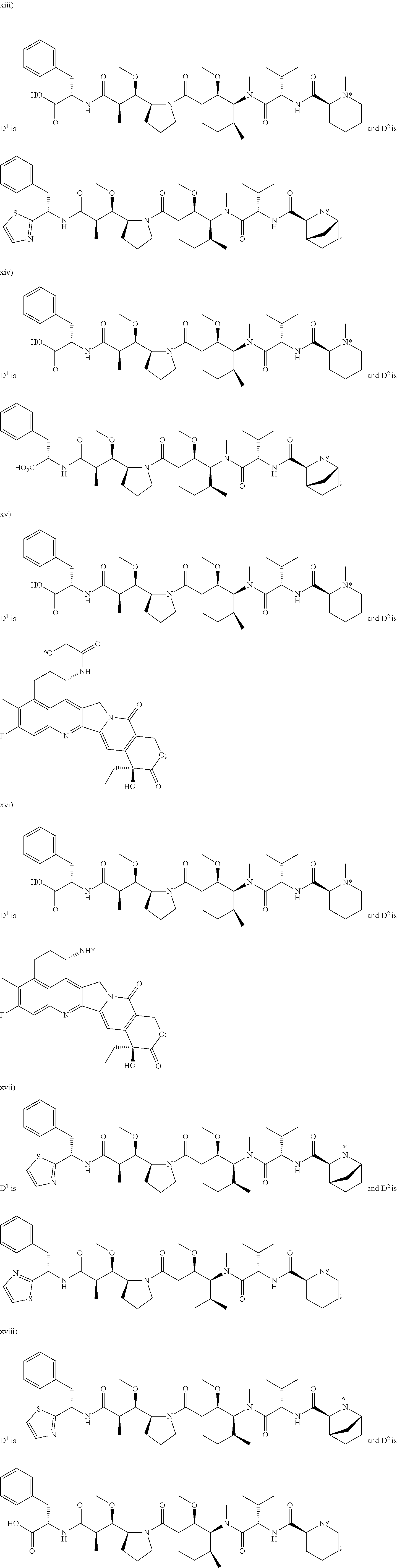

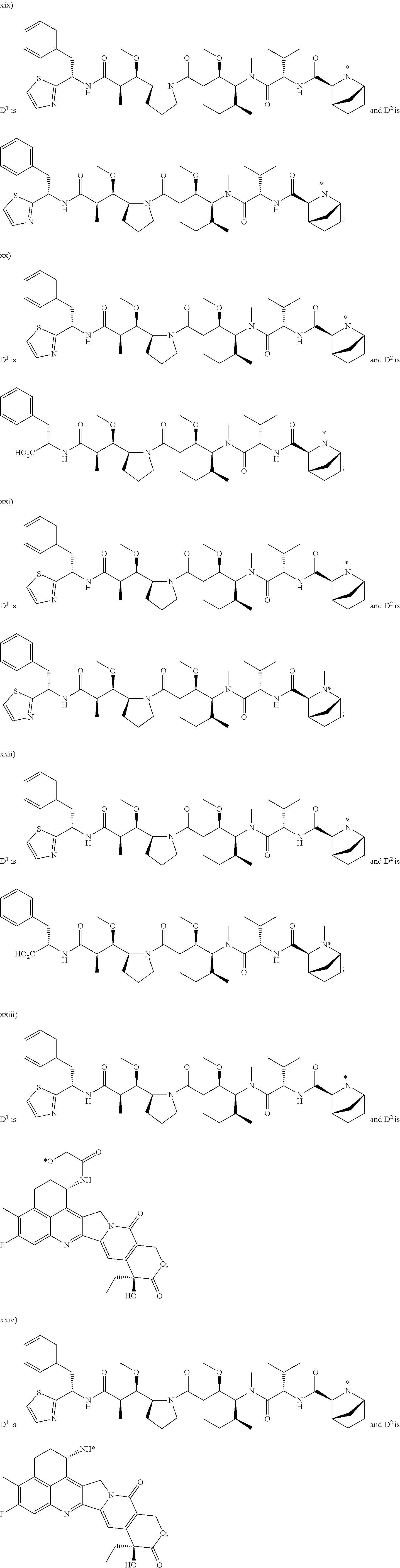

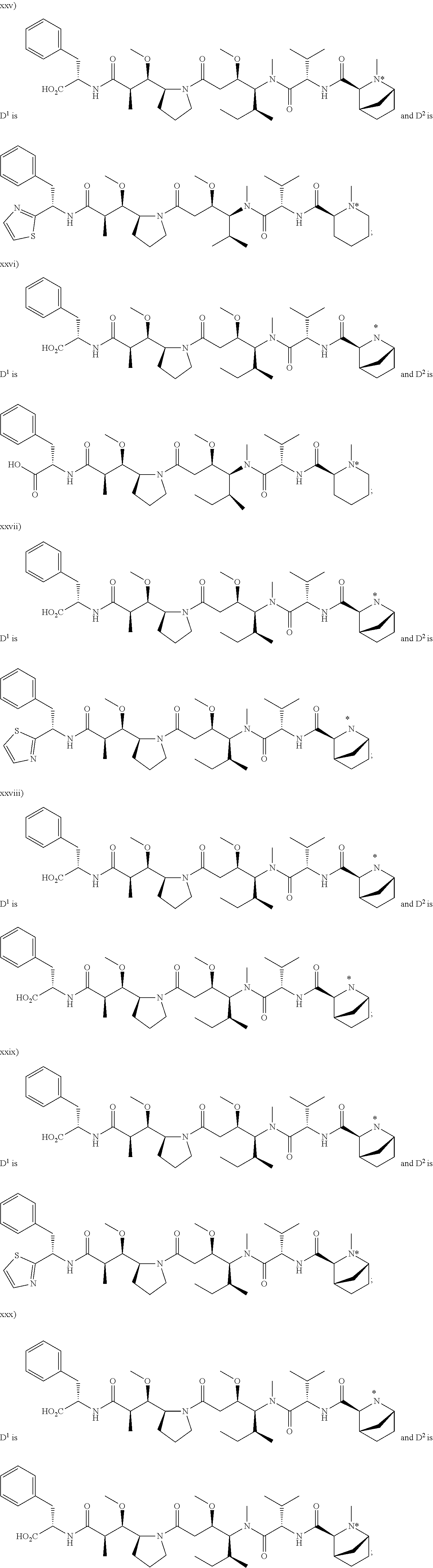

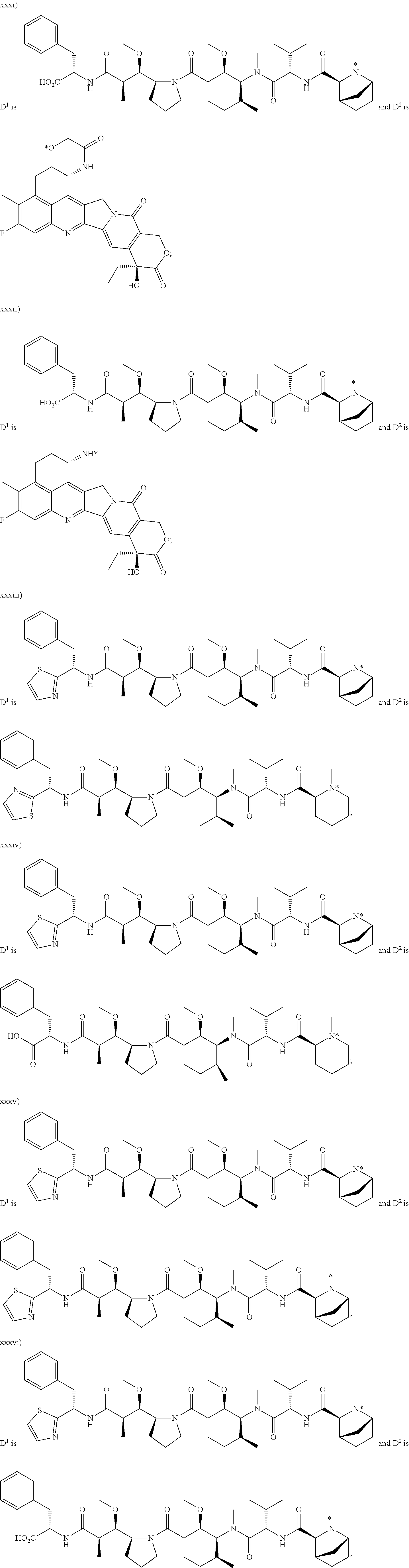

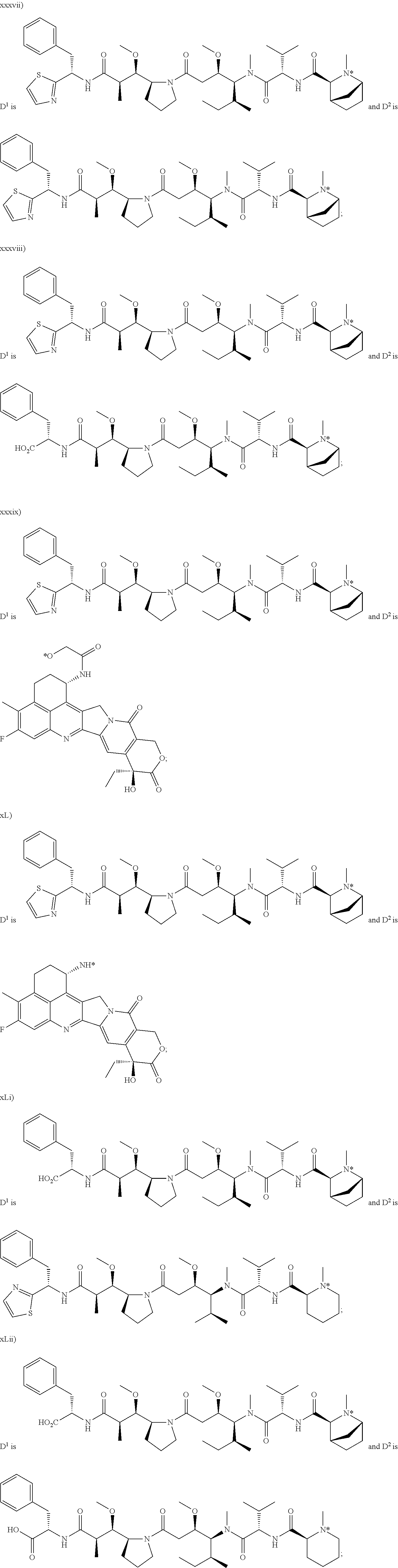

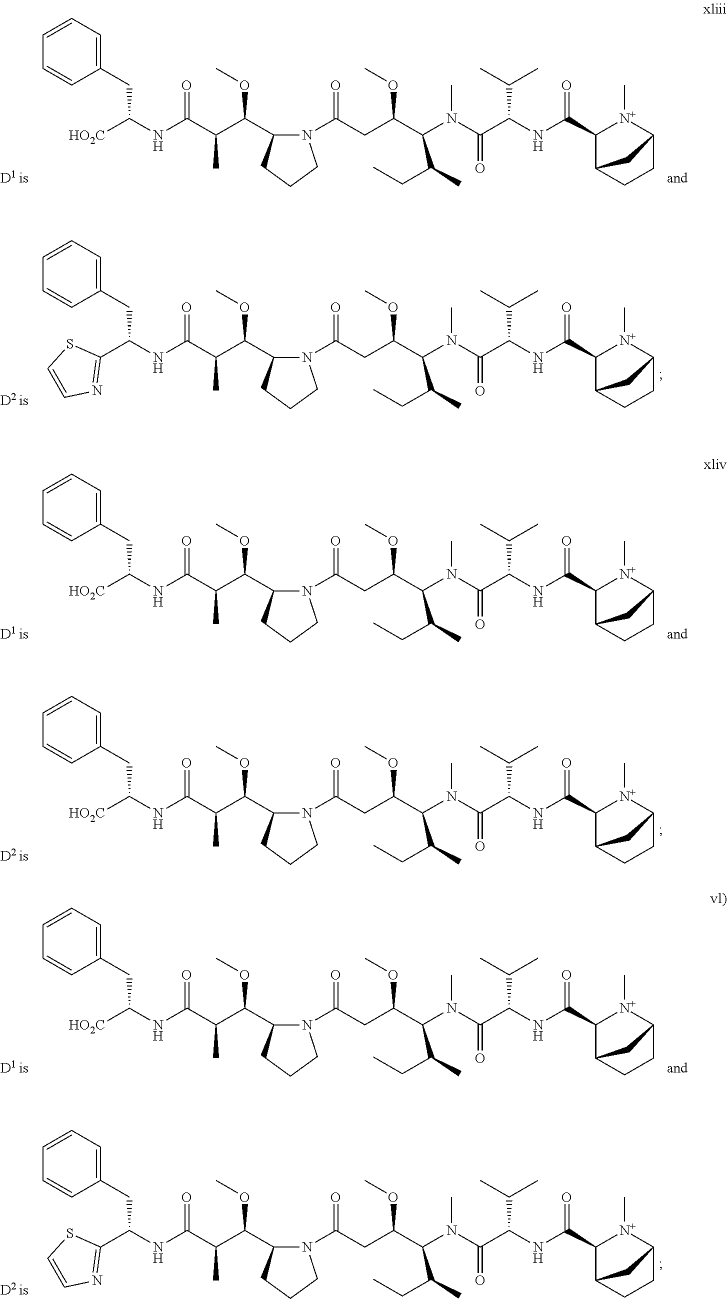

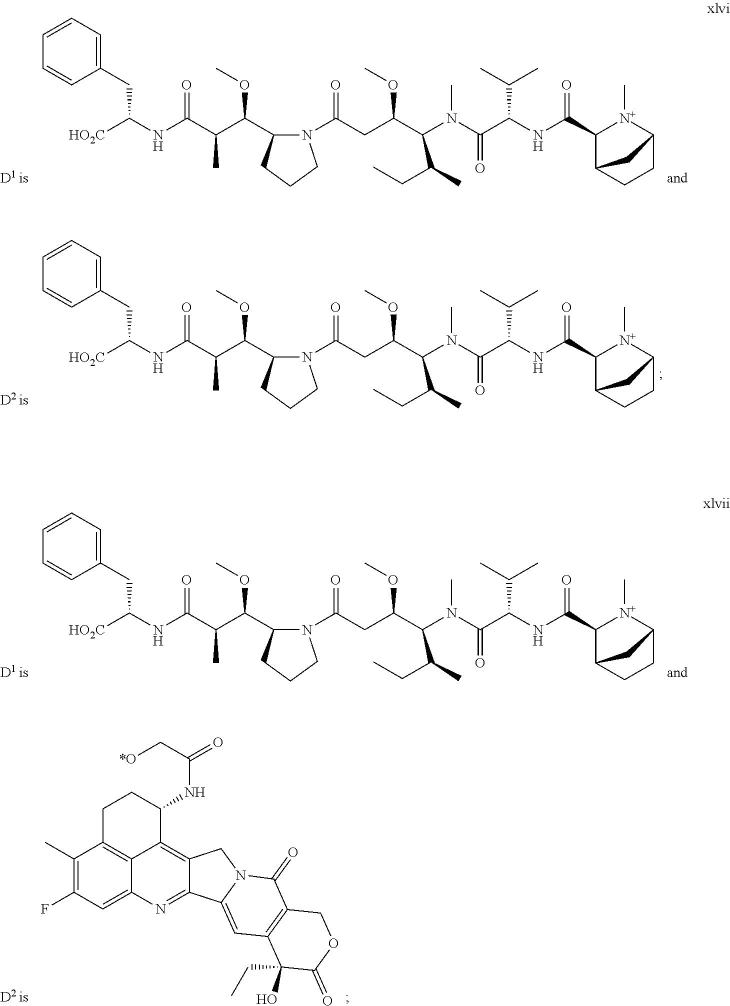

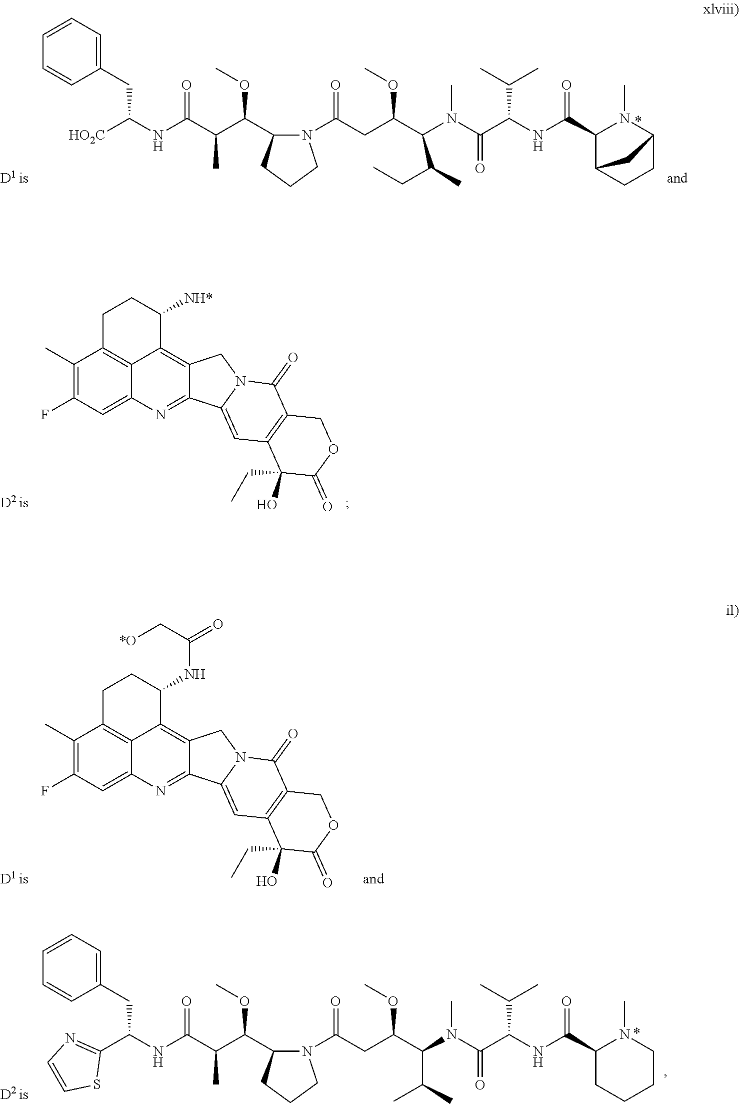

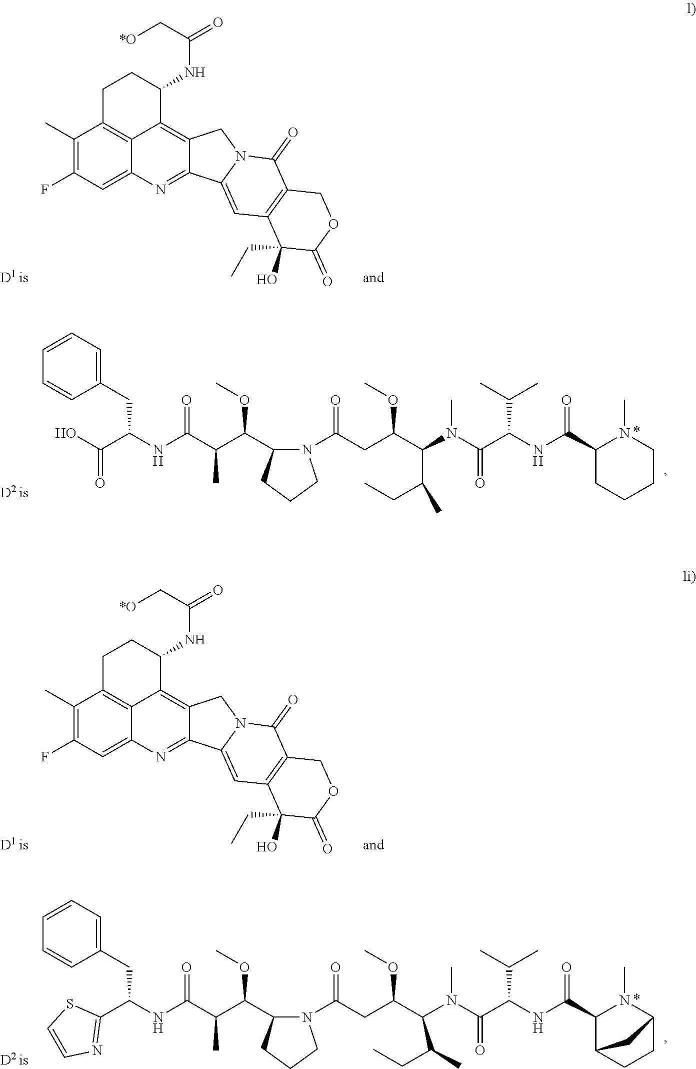

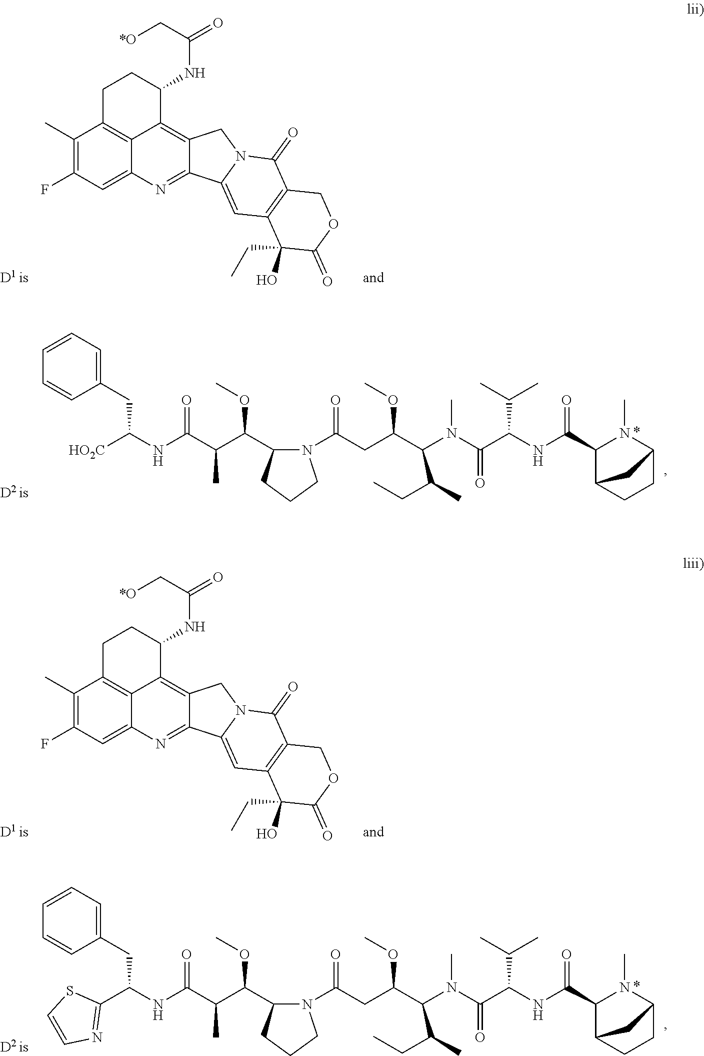

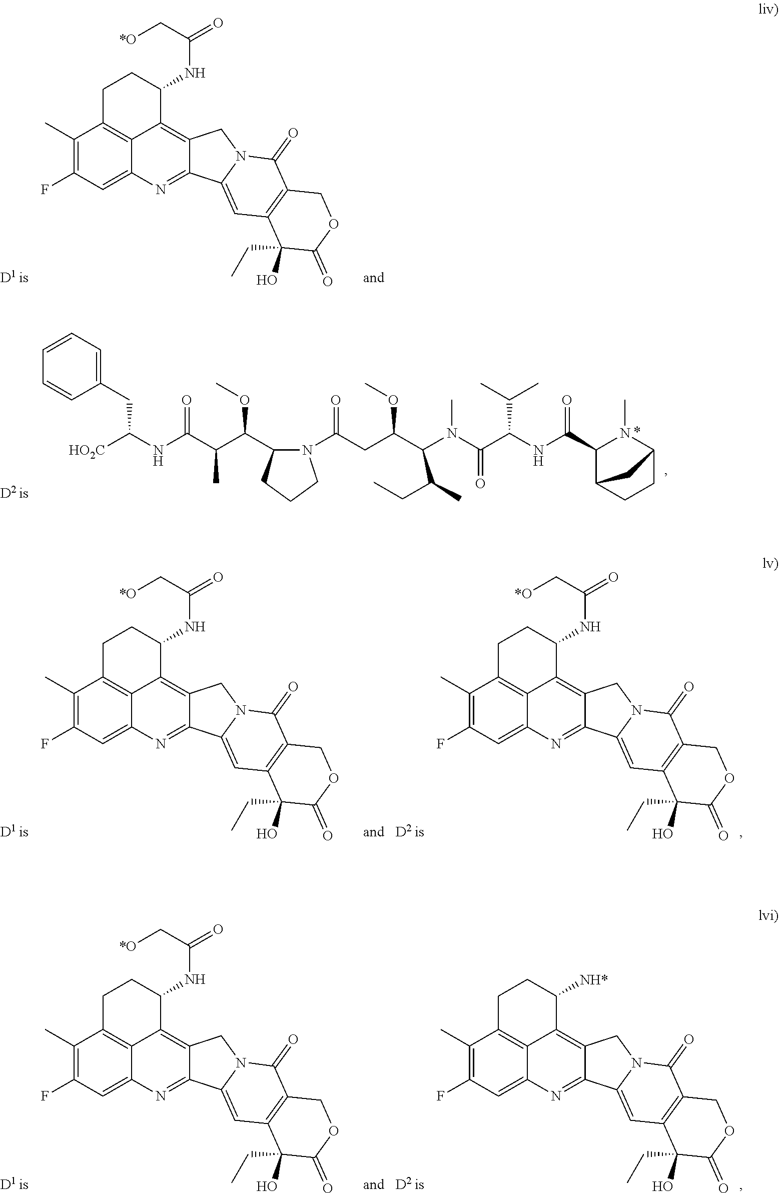

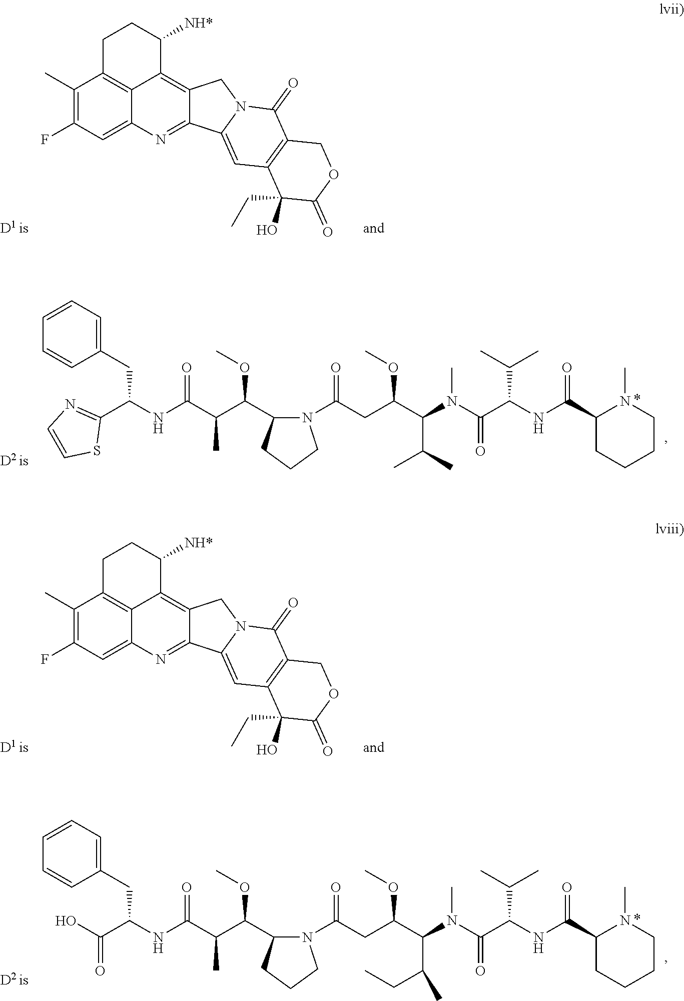

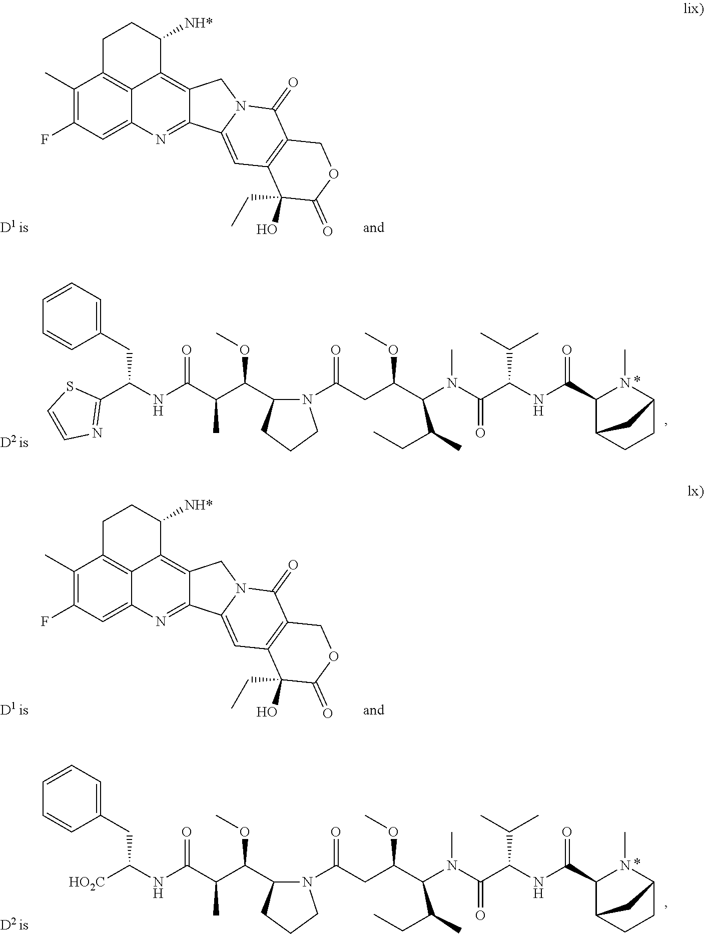

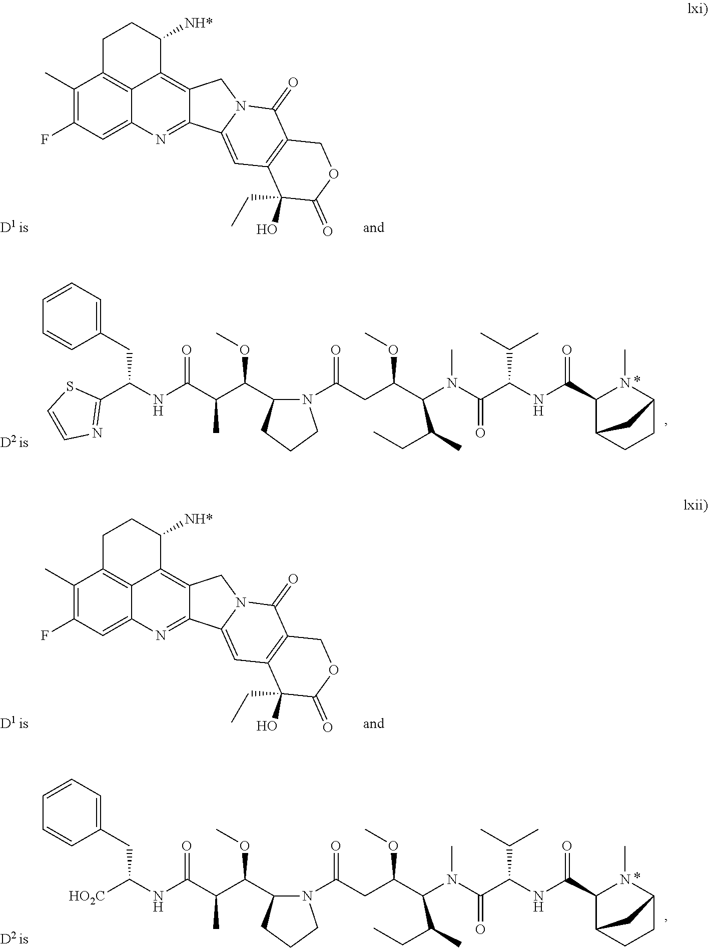

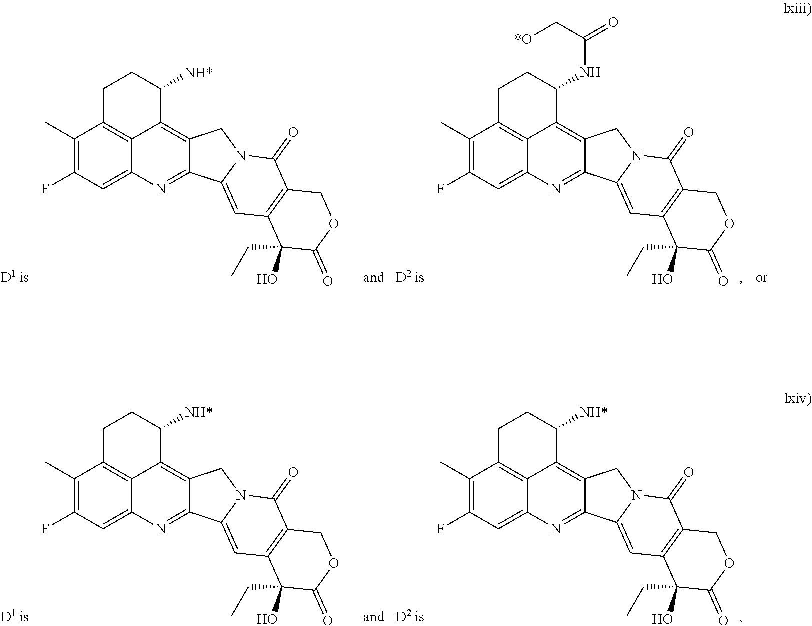

wherein * indicates the point of connection to the remainder of the molecule.

In an embodiment, A¹-D¹ and A²-D² are independently selected from the group consisting of:

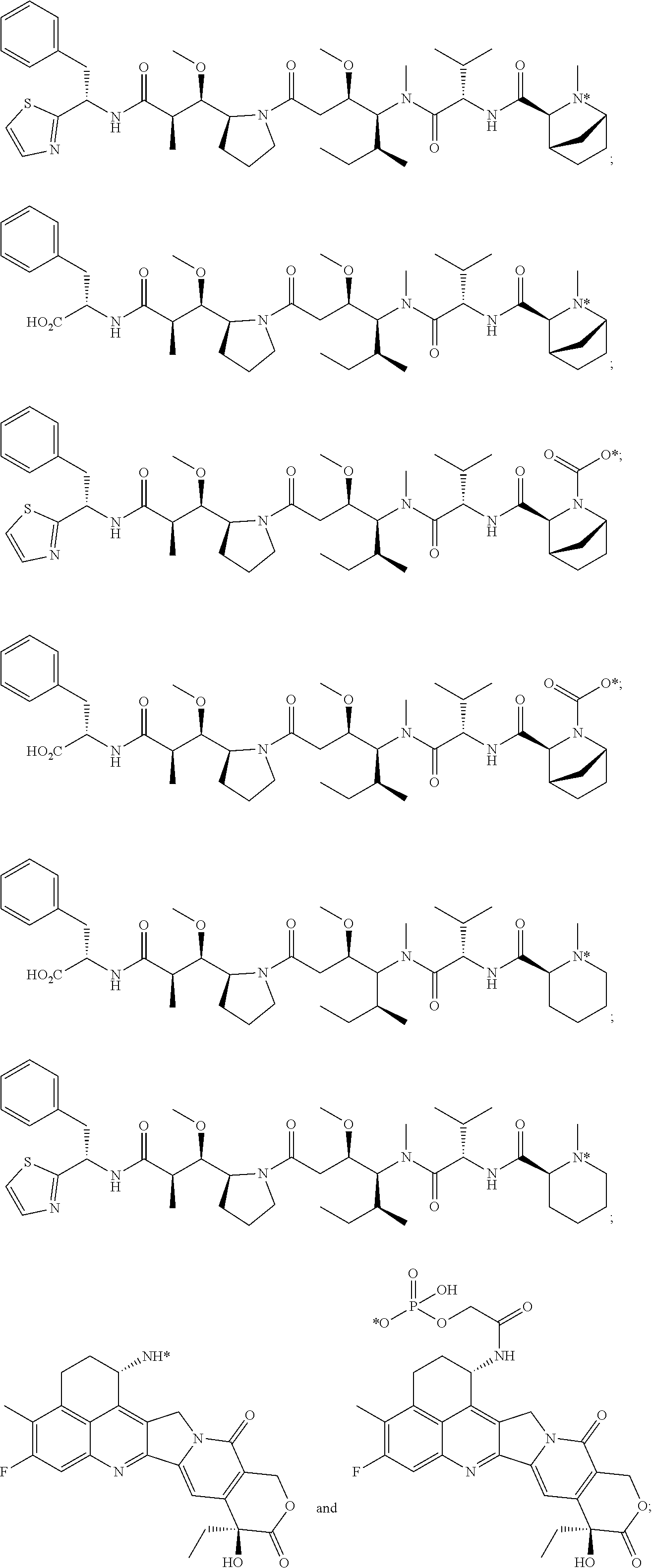

wherein * indicates the point of connection to the remainder of the molecule.
In an embodiment:

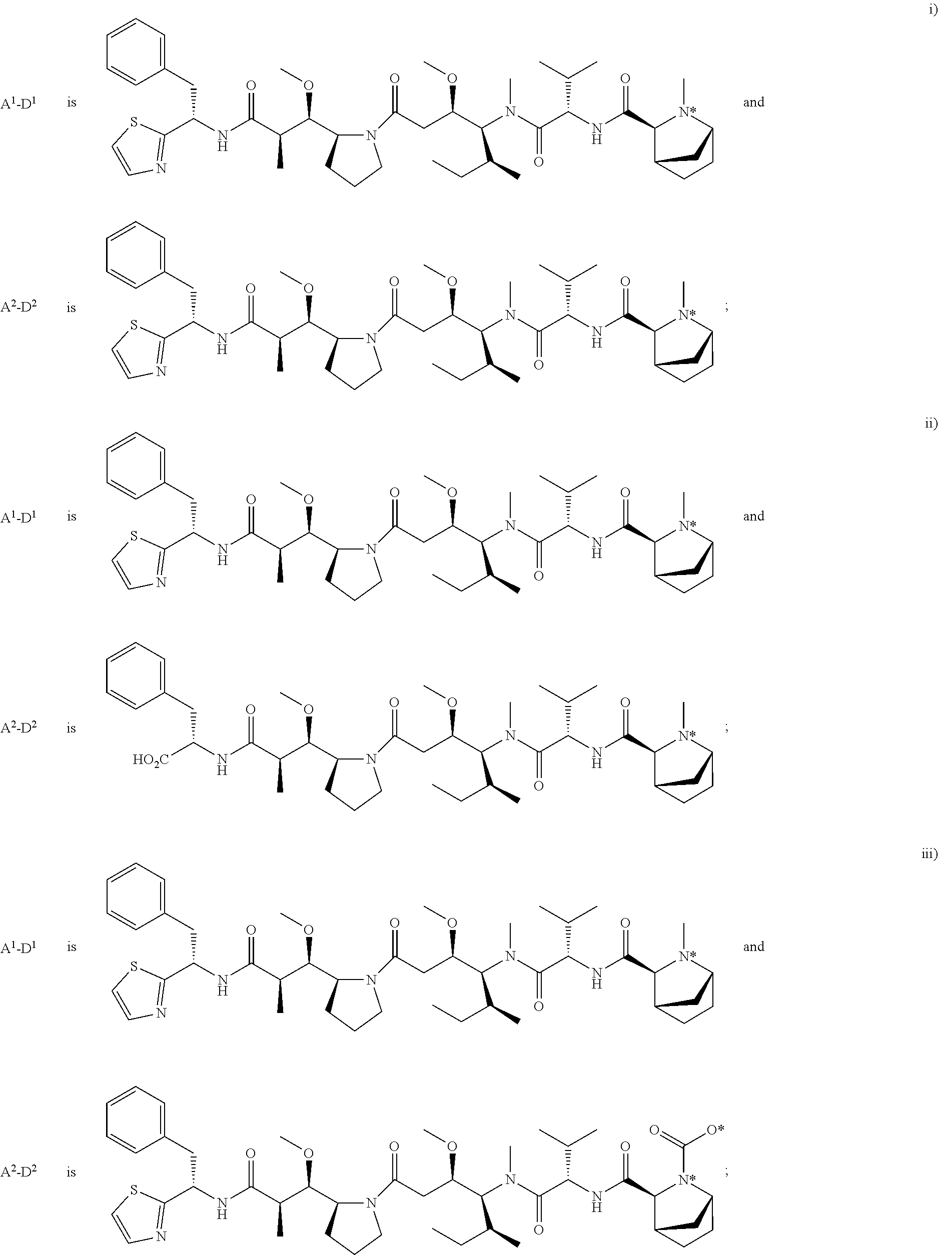

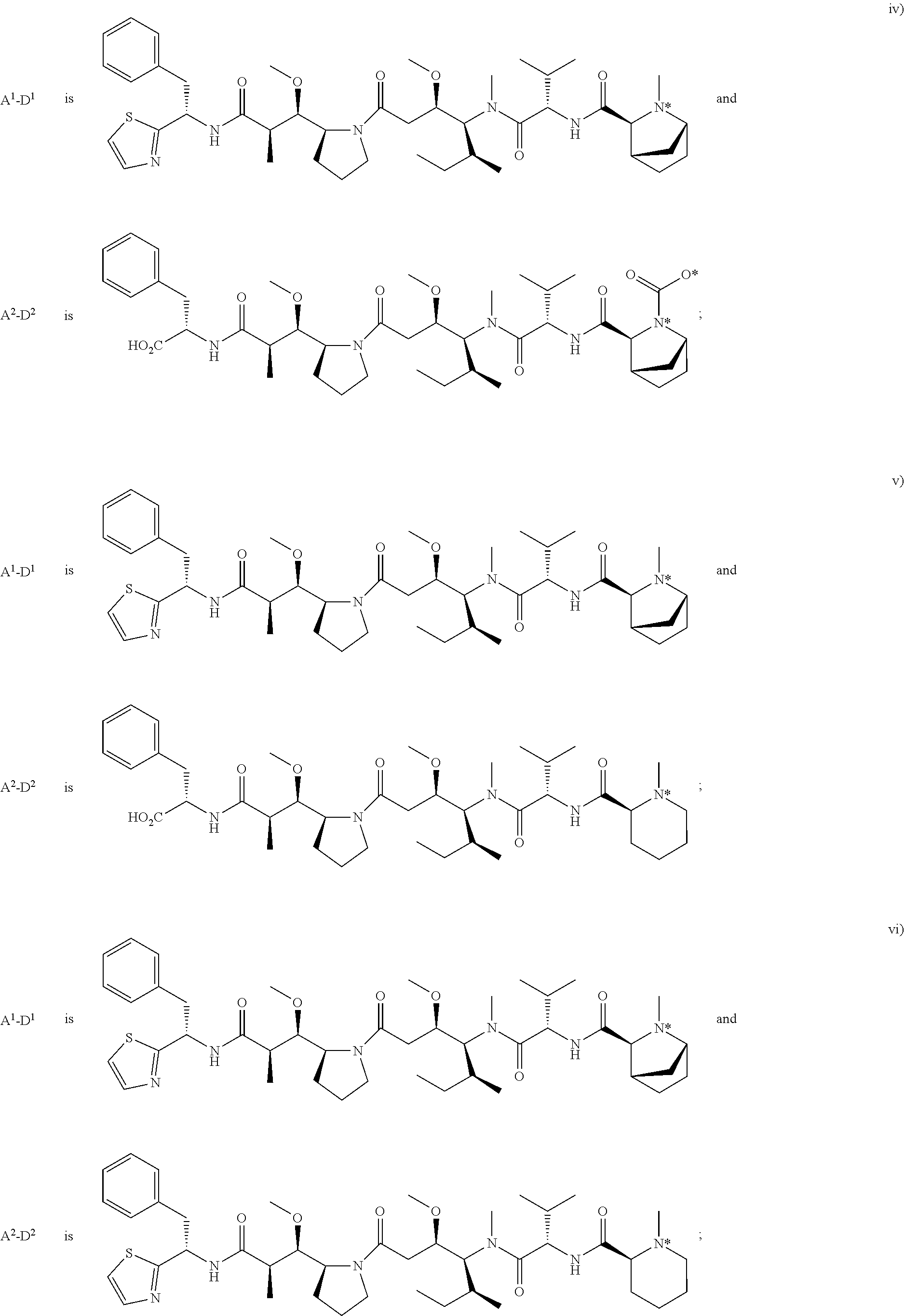

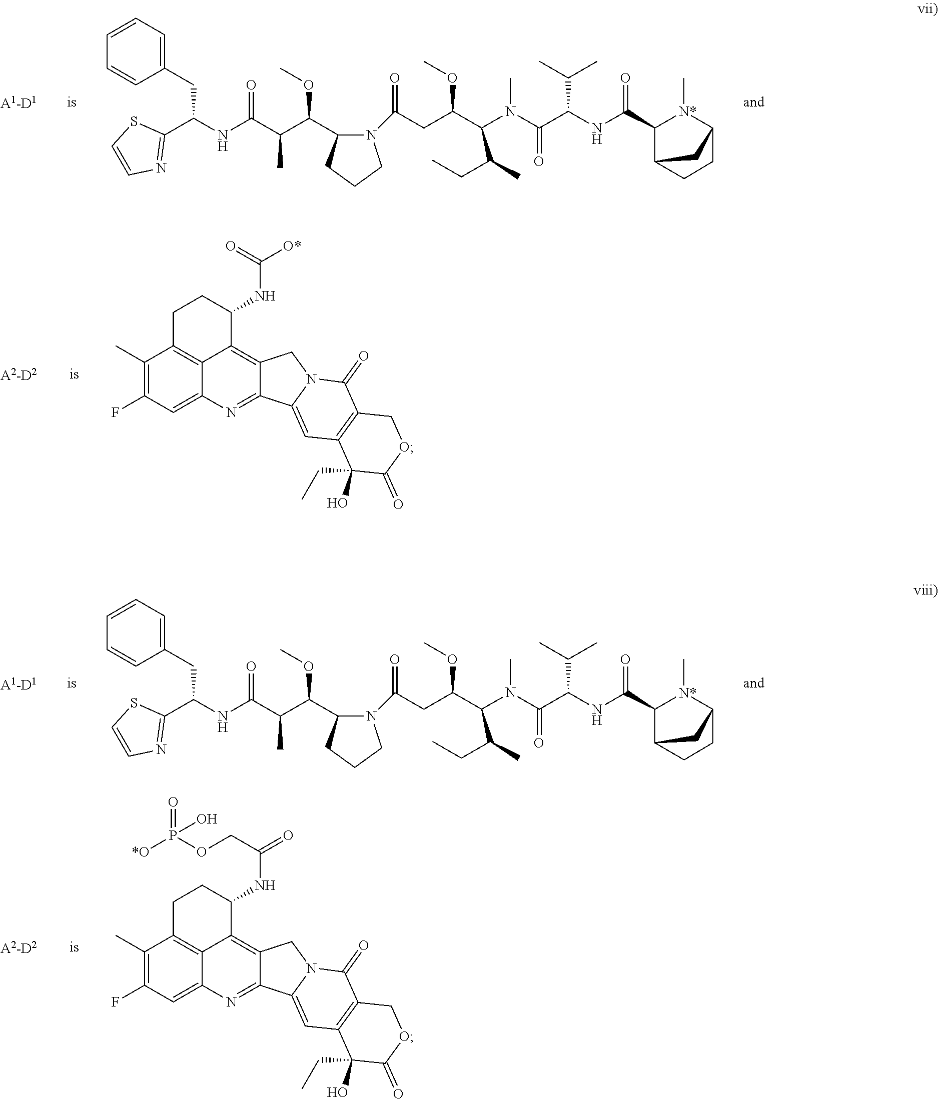

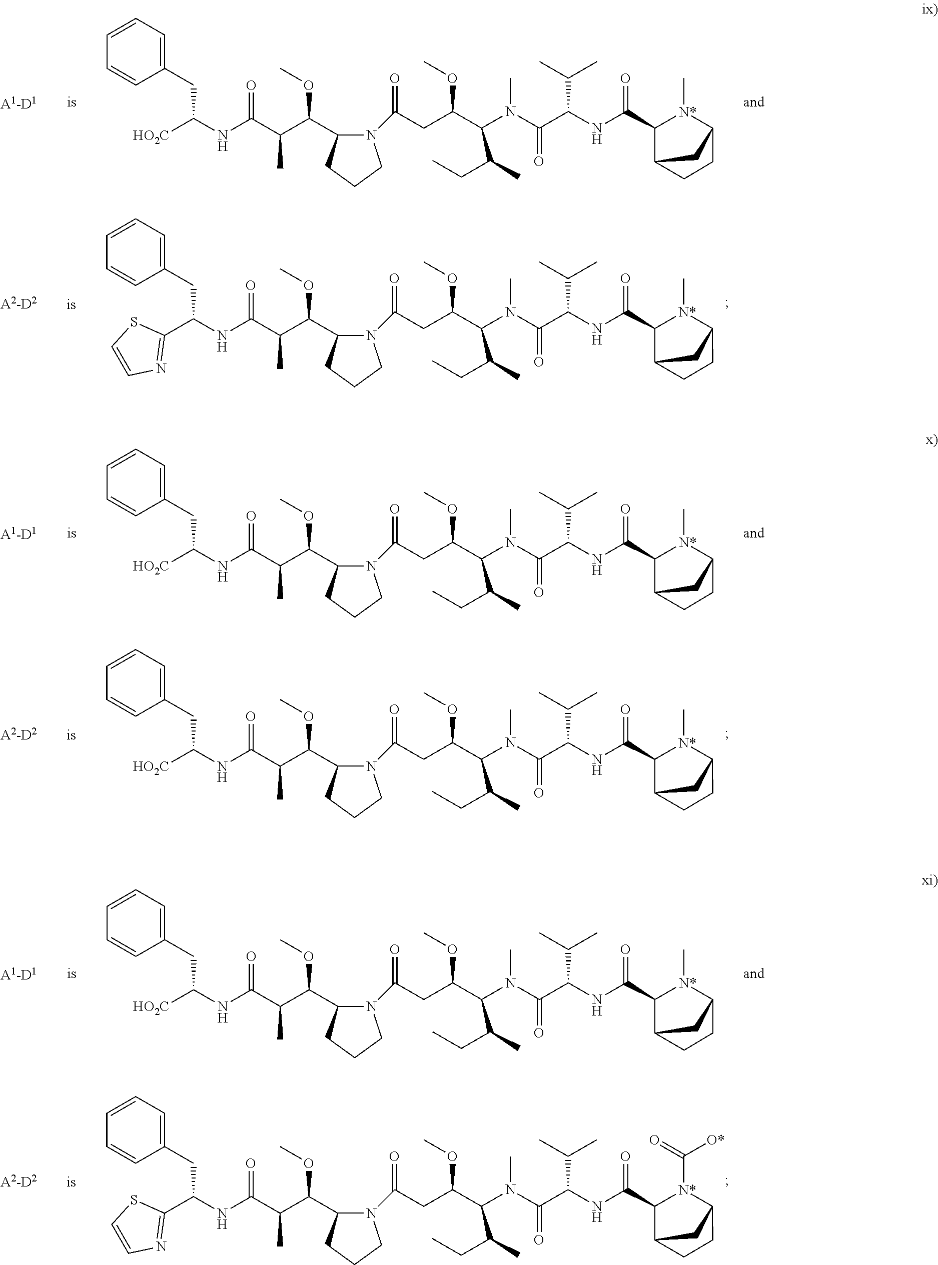

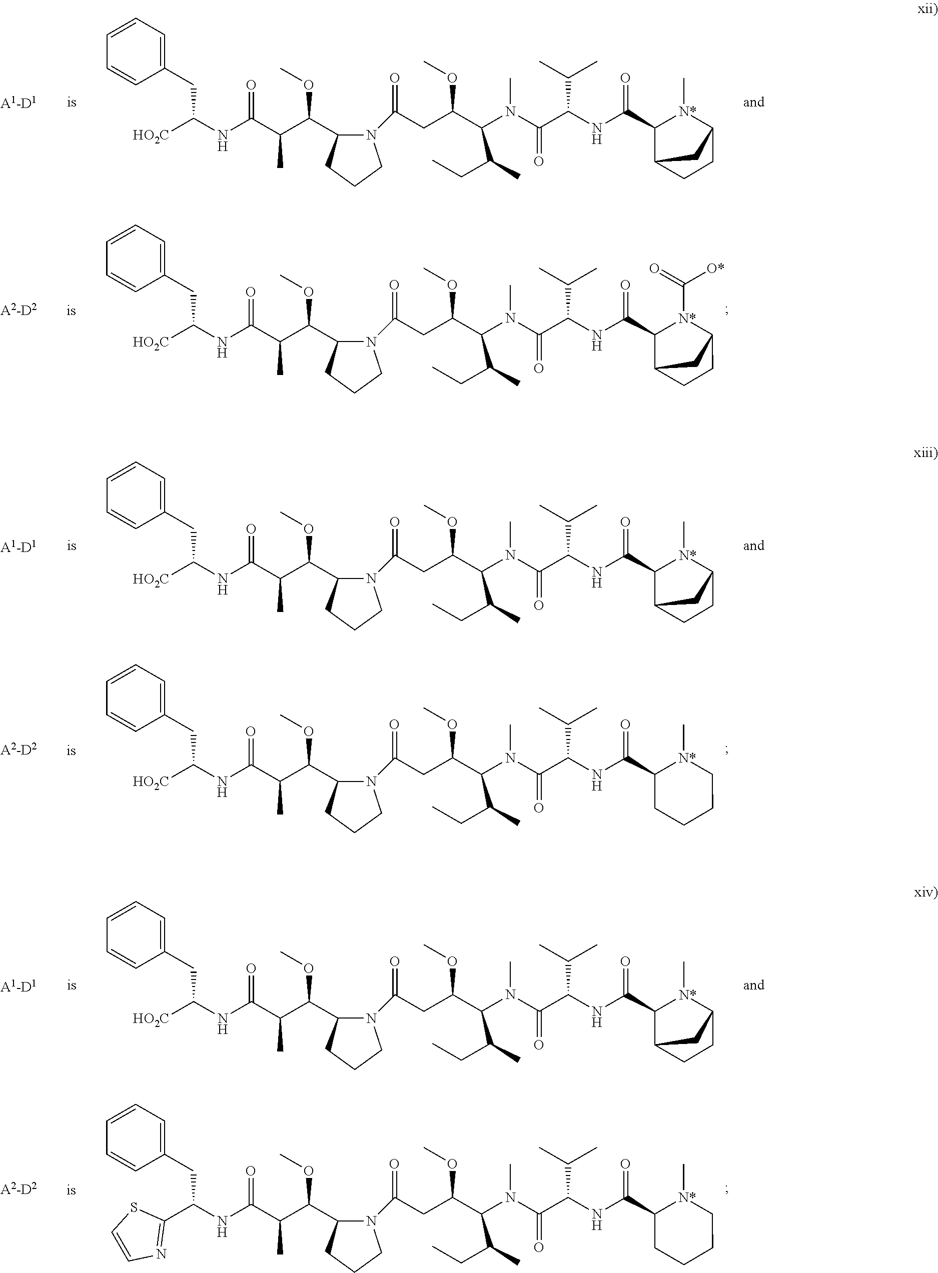

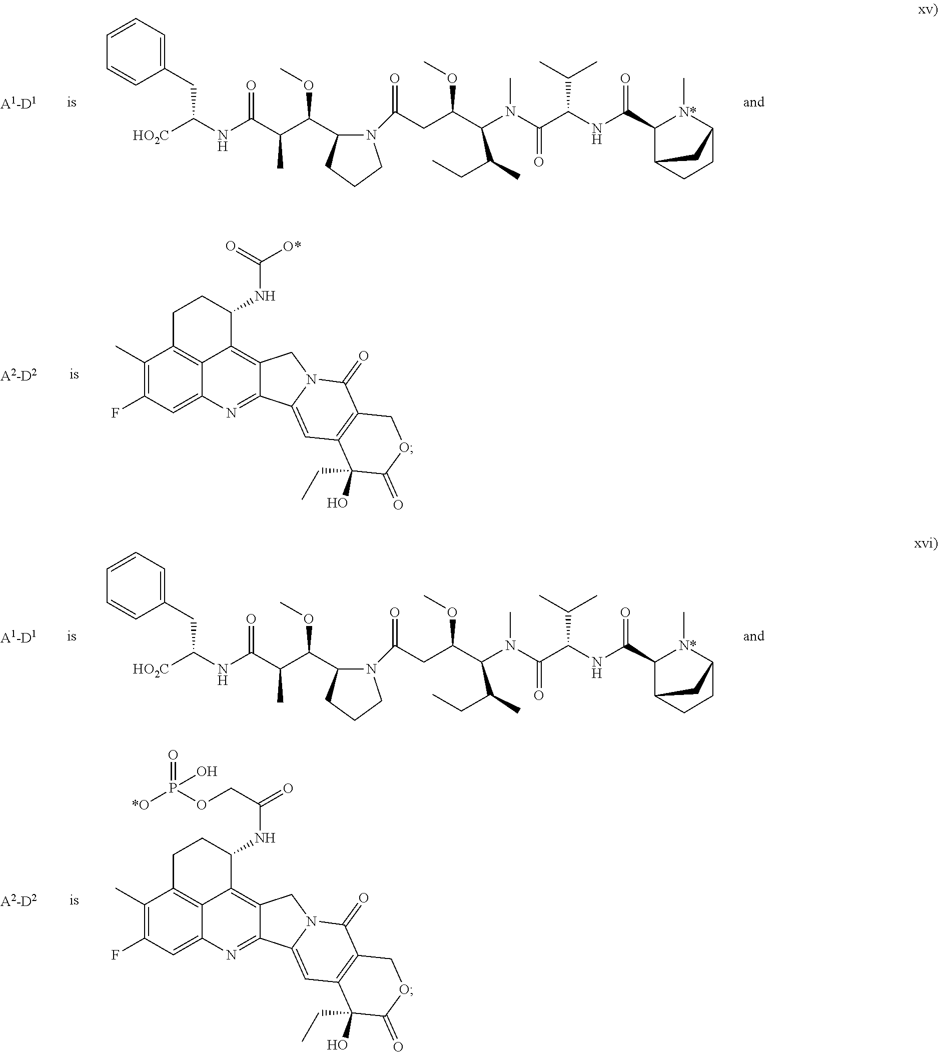

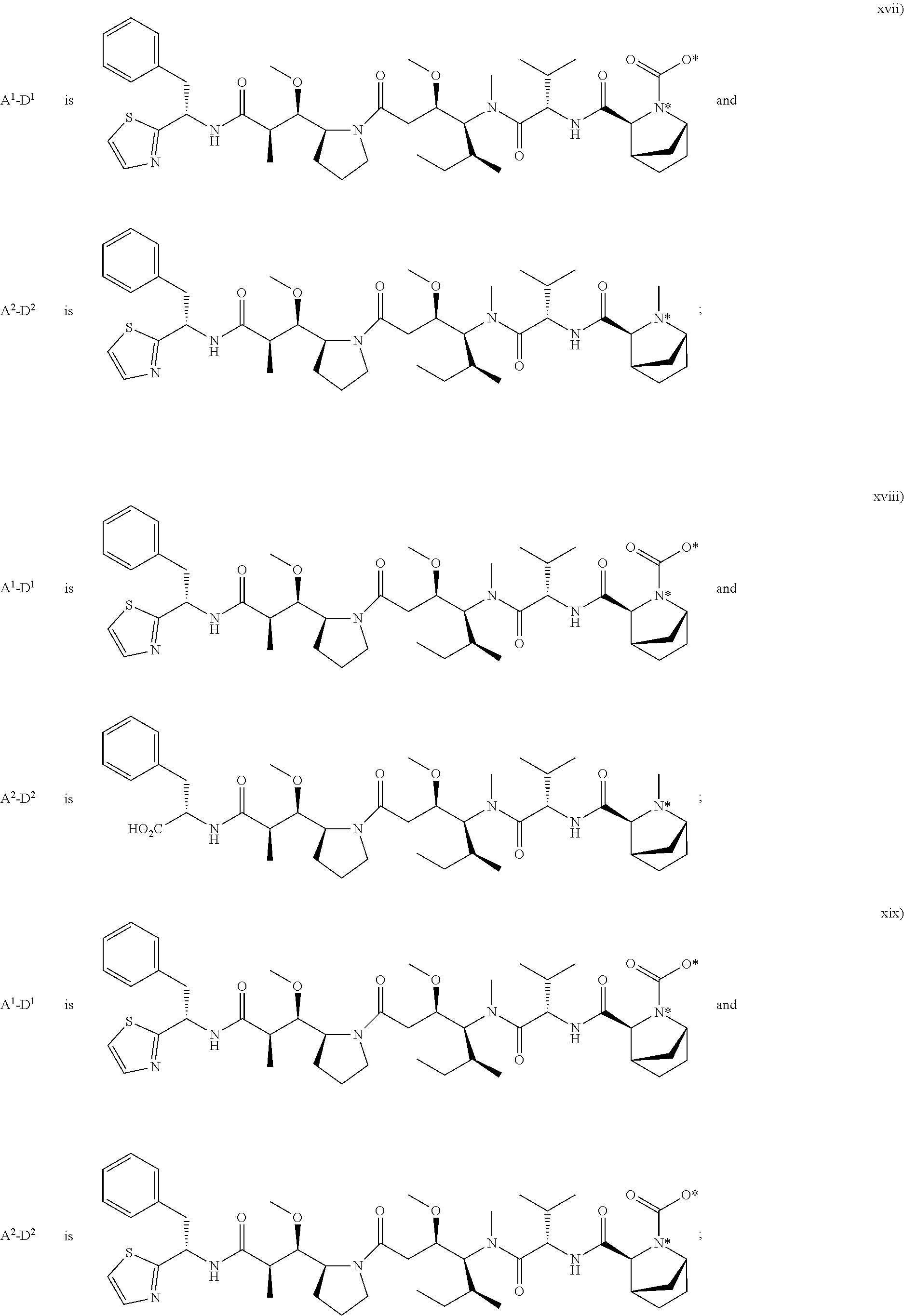

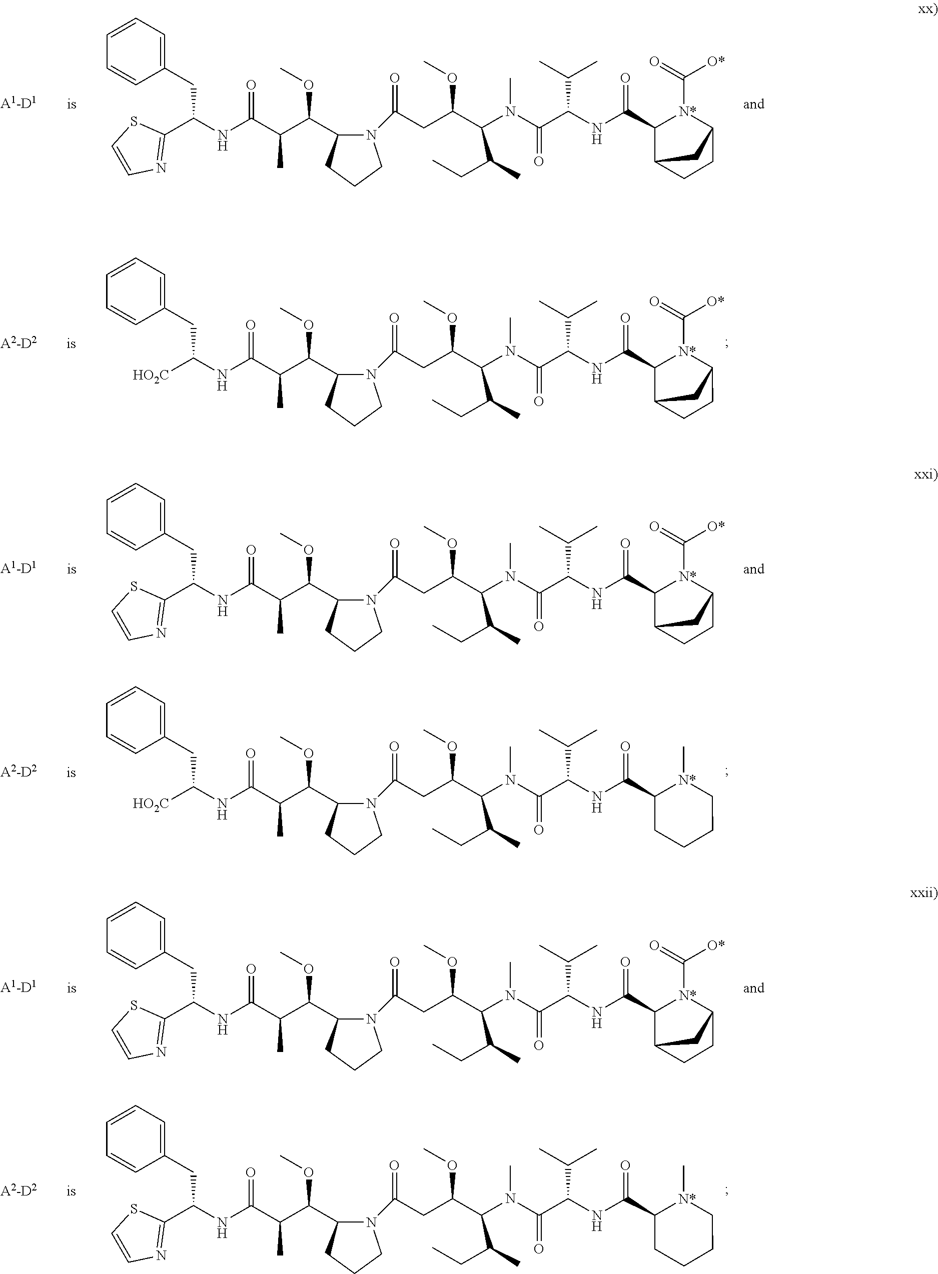

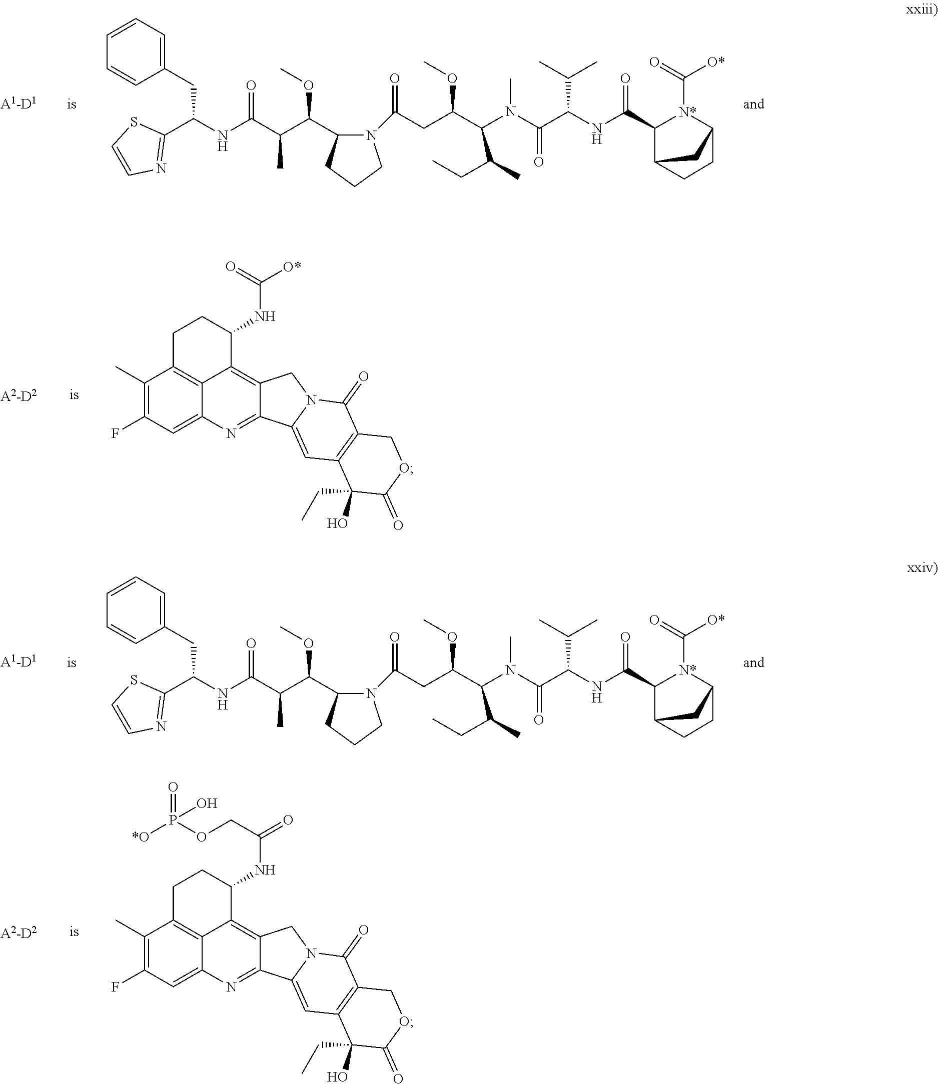

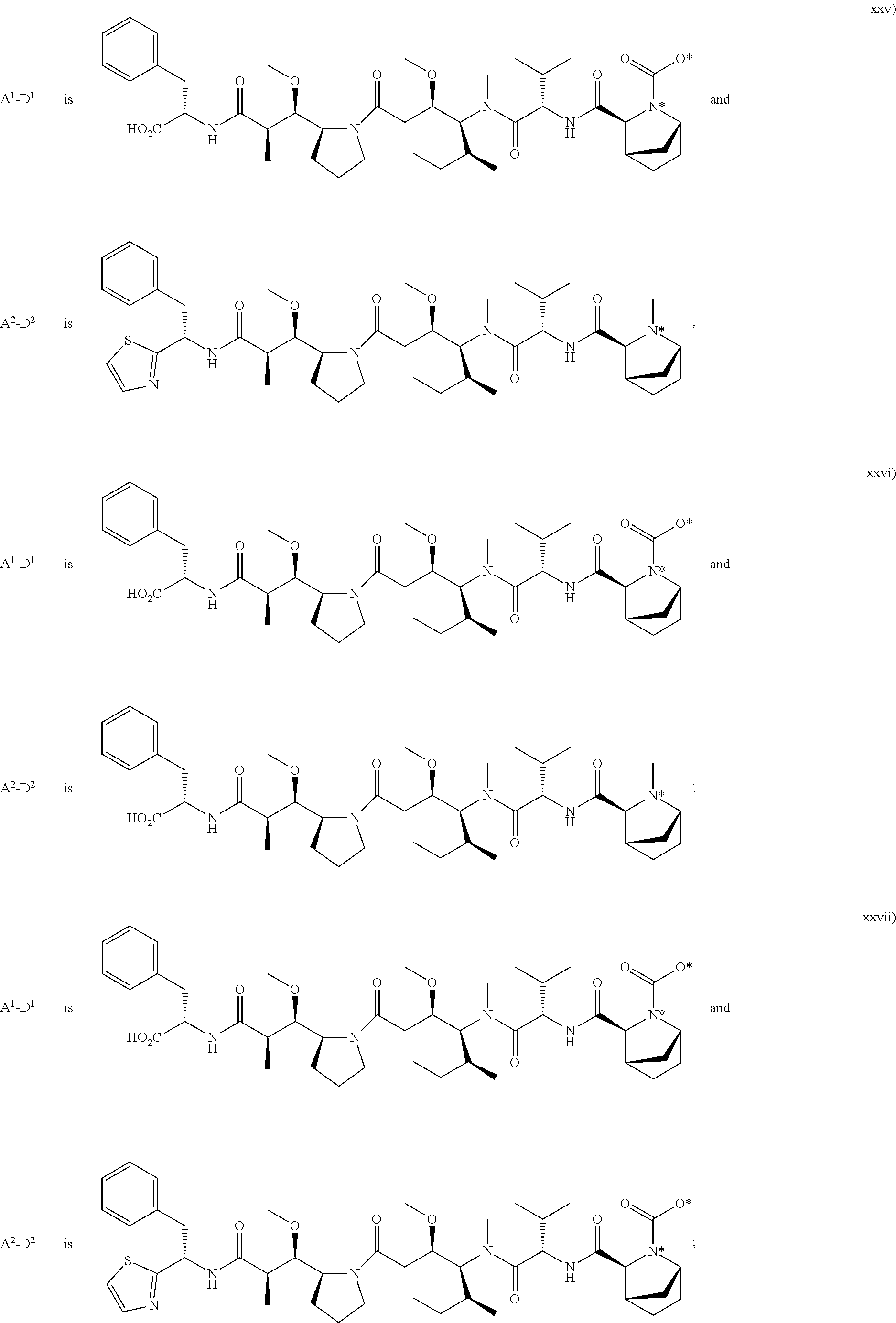

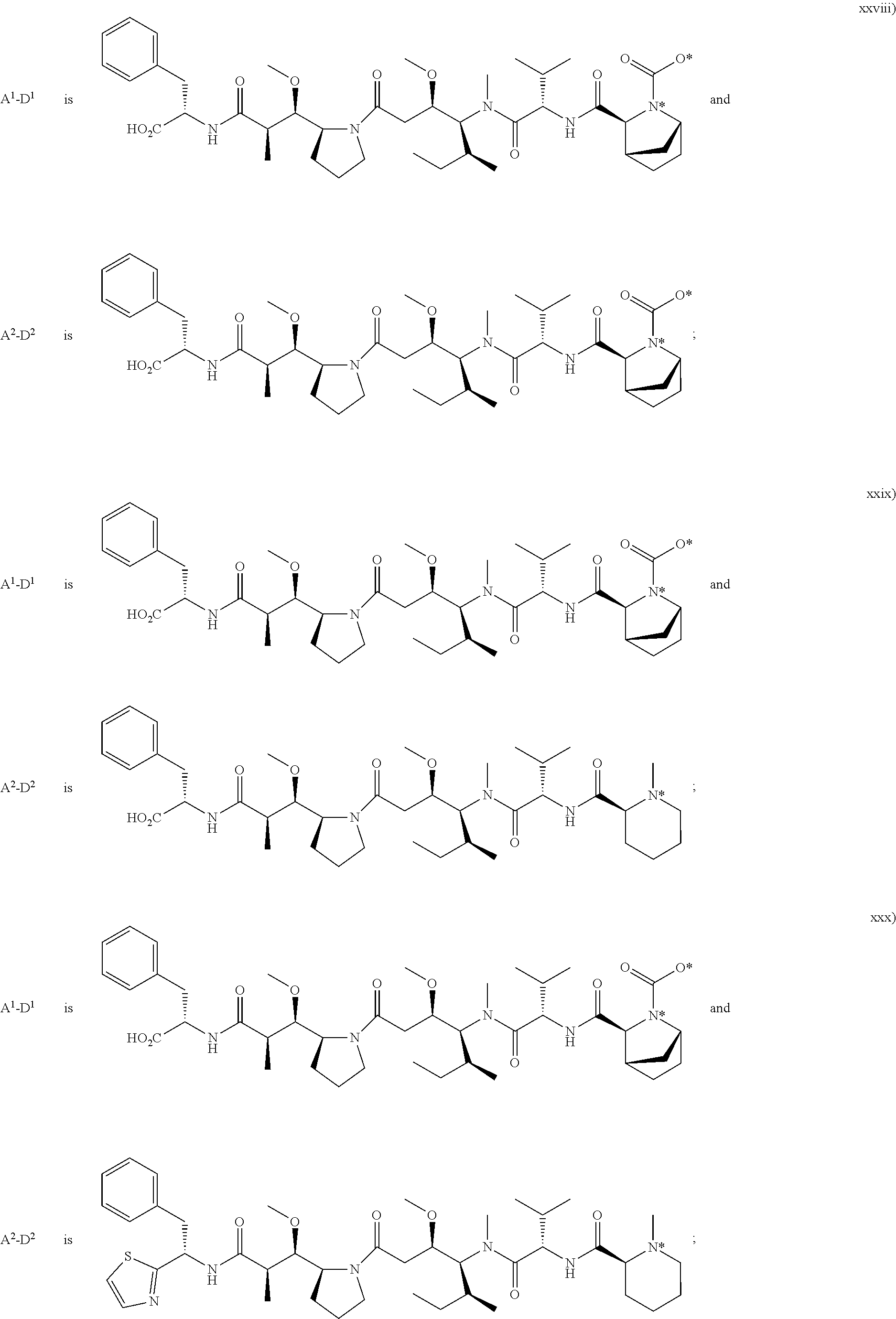

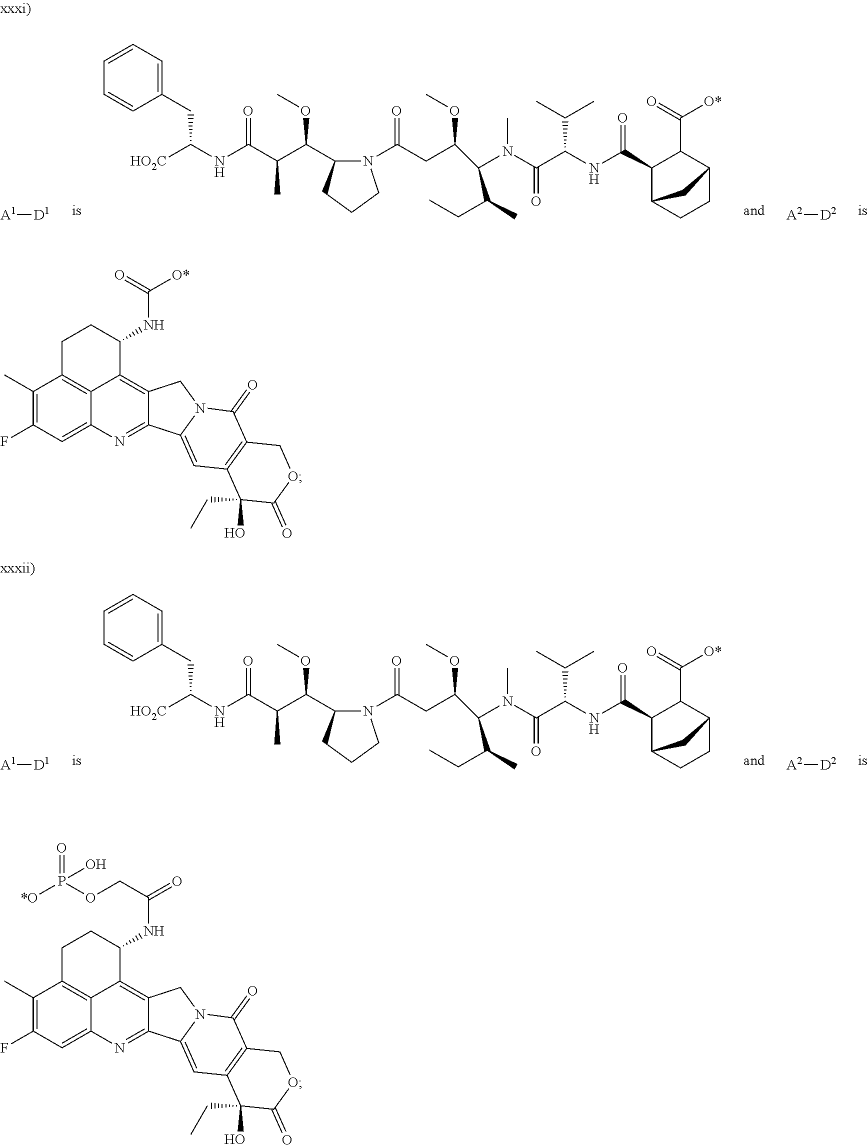

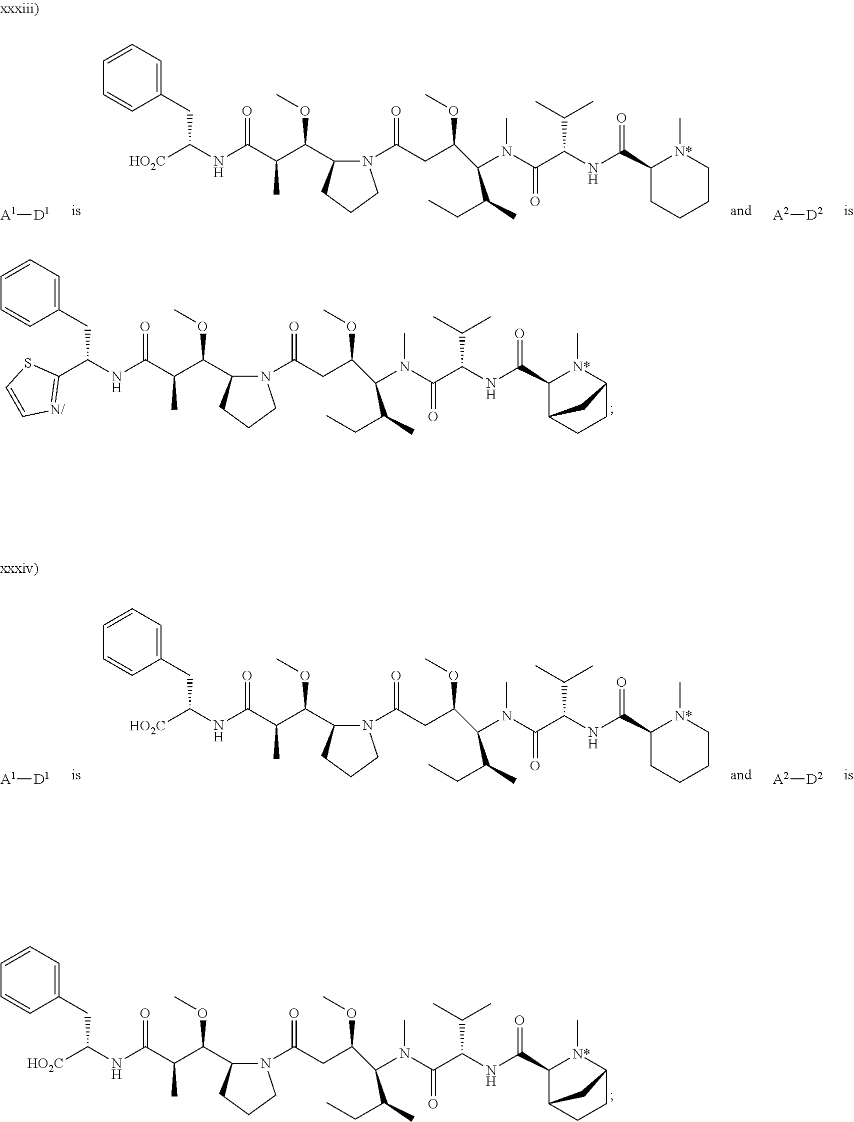

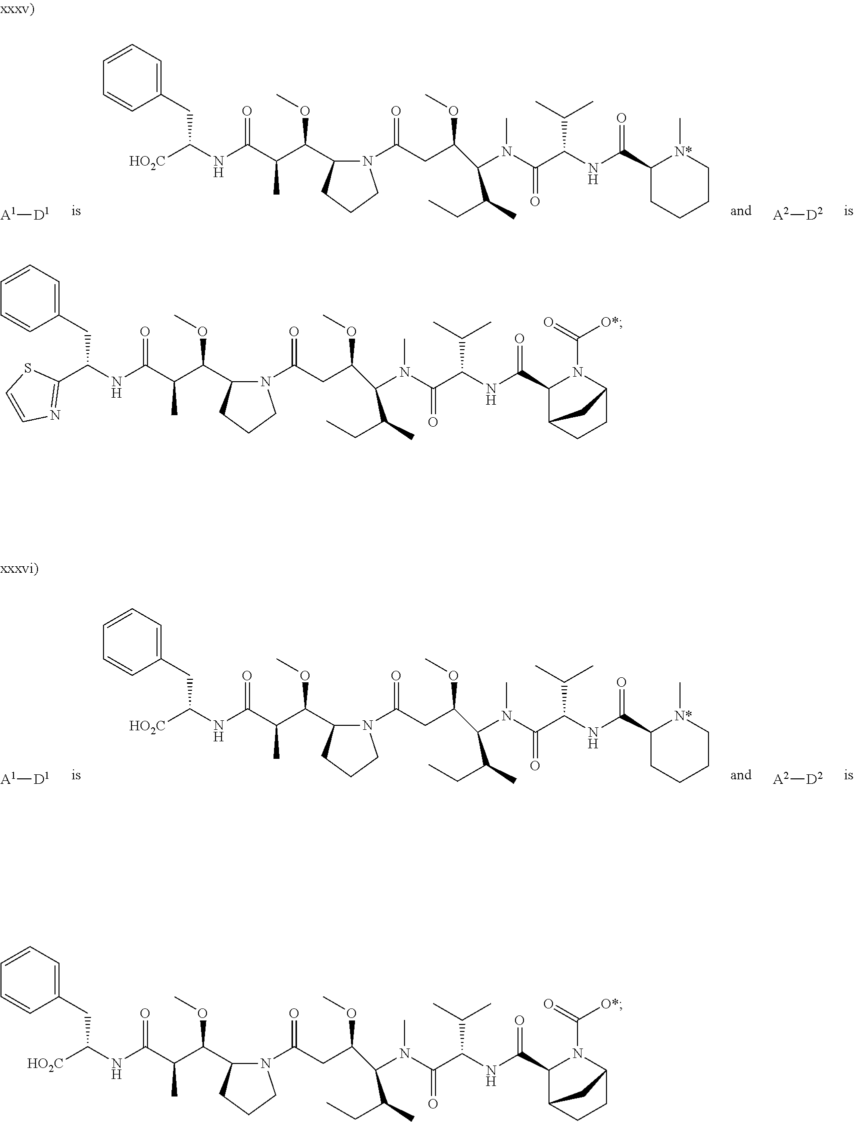

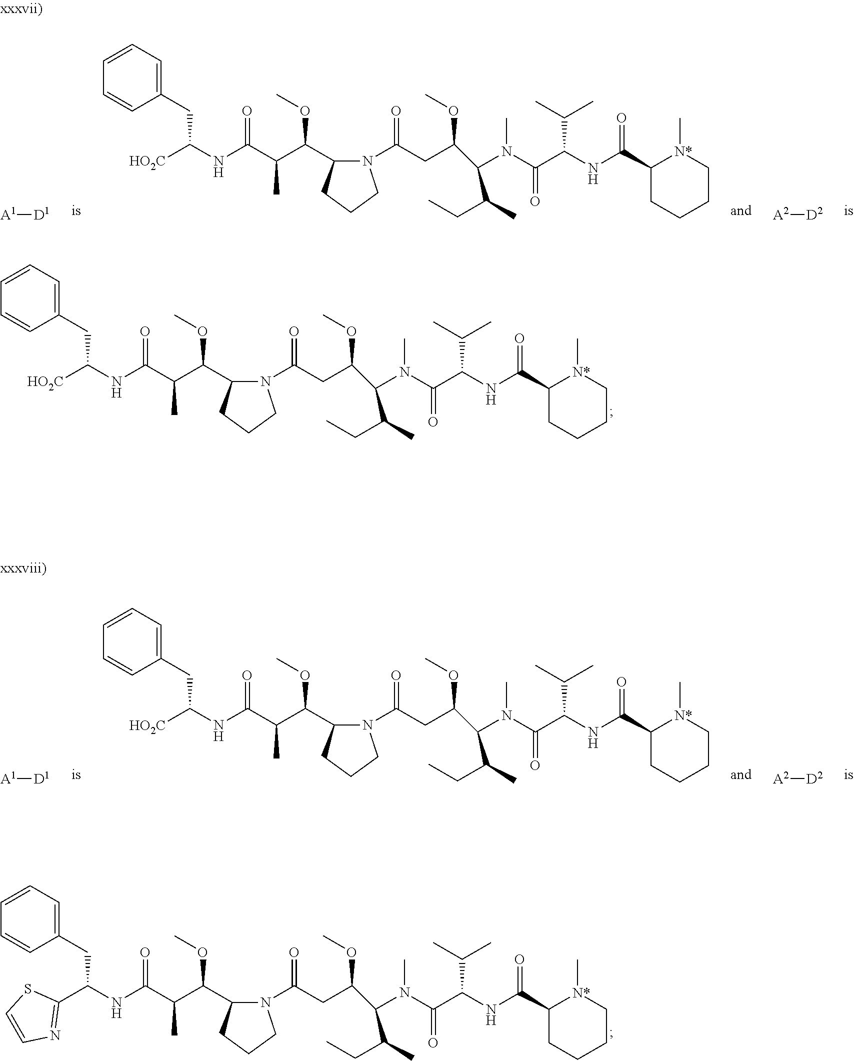

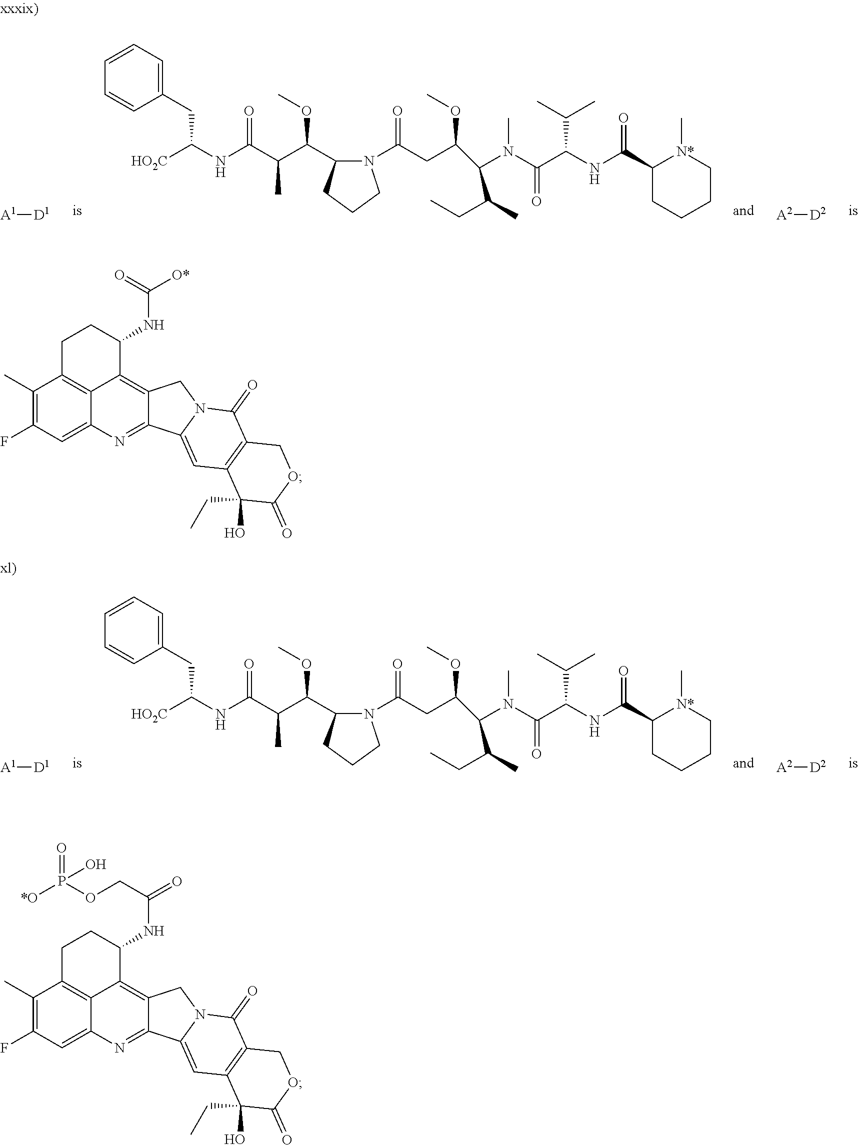

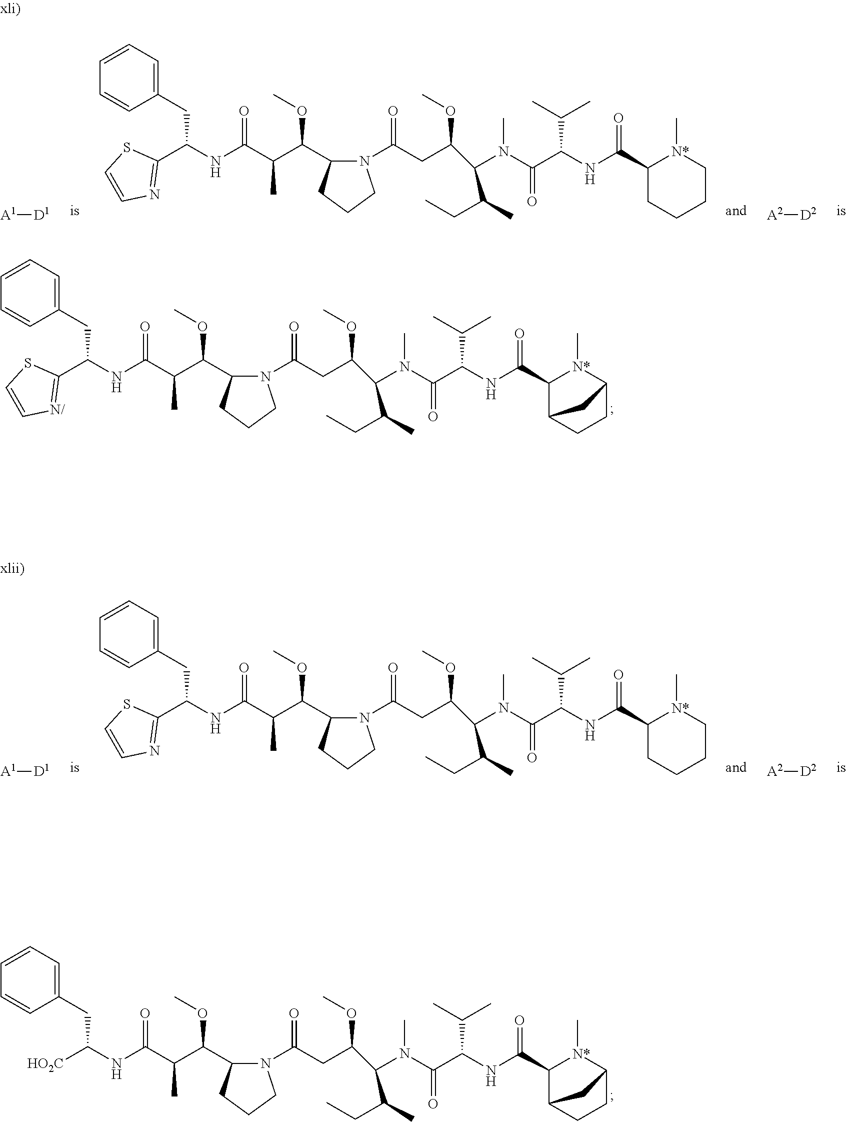

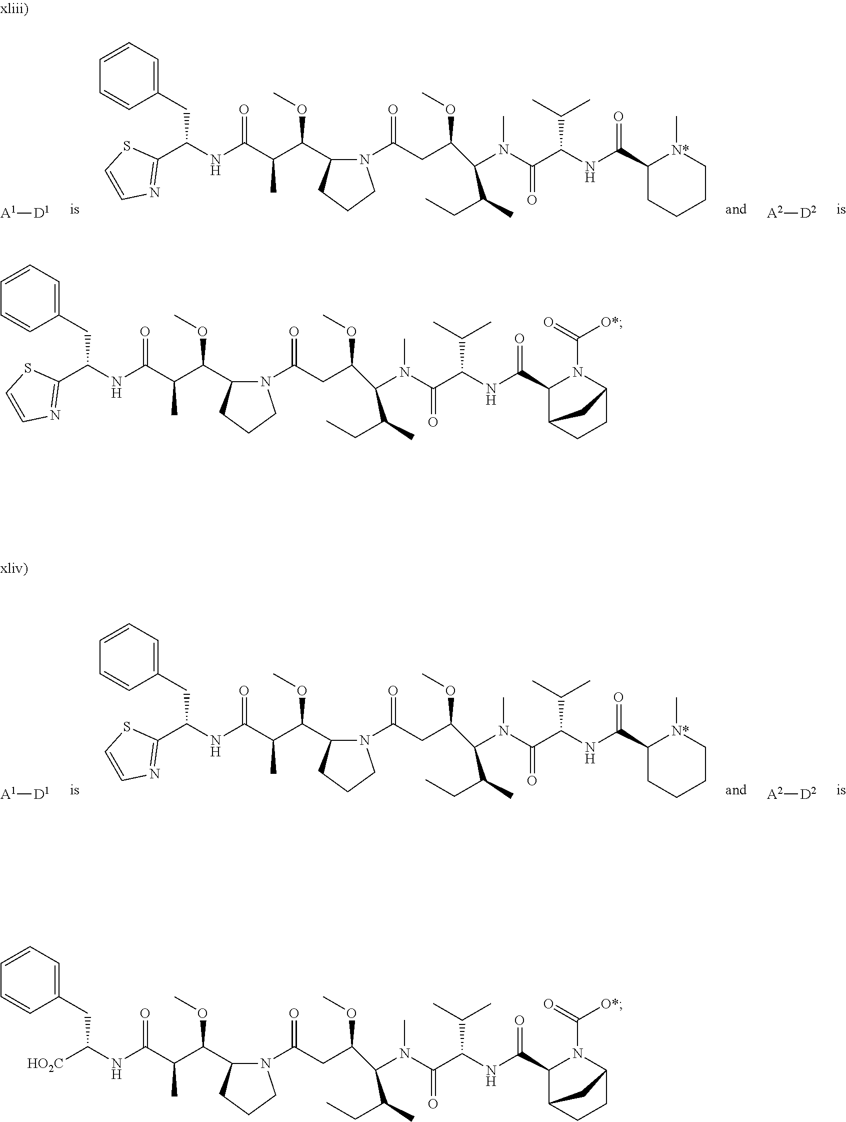

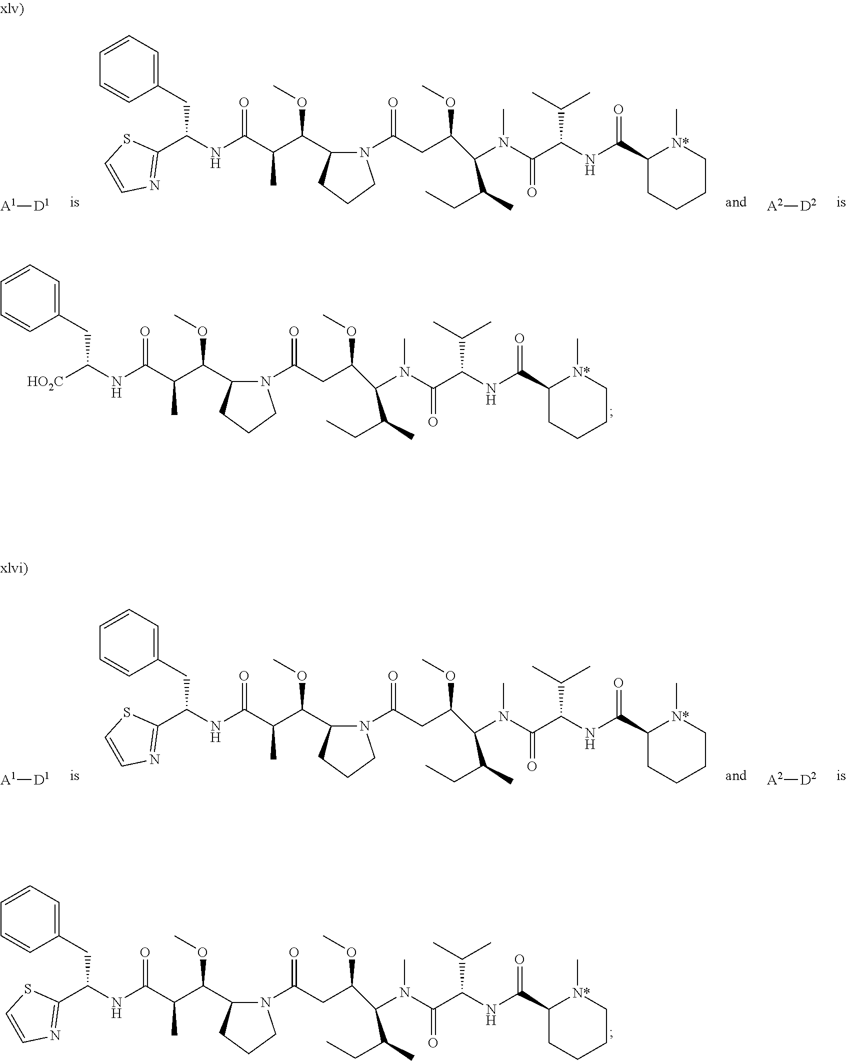

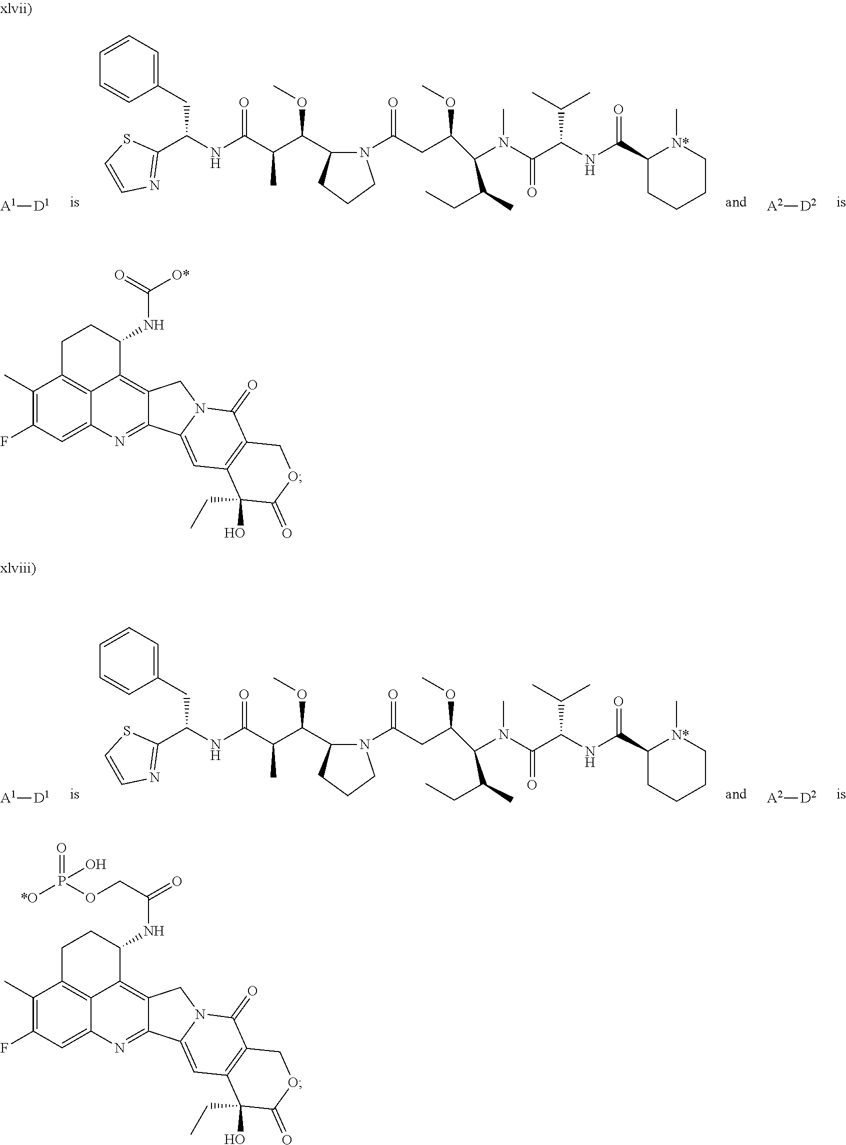

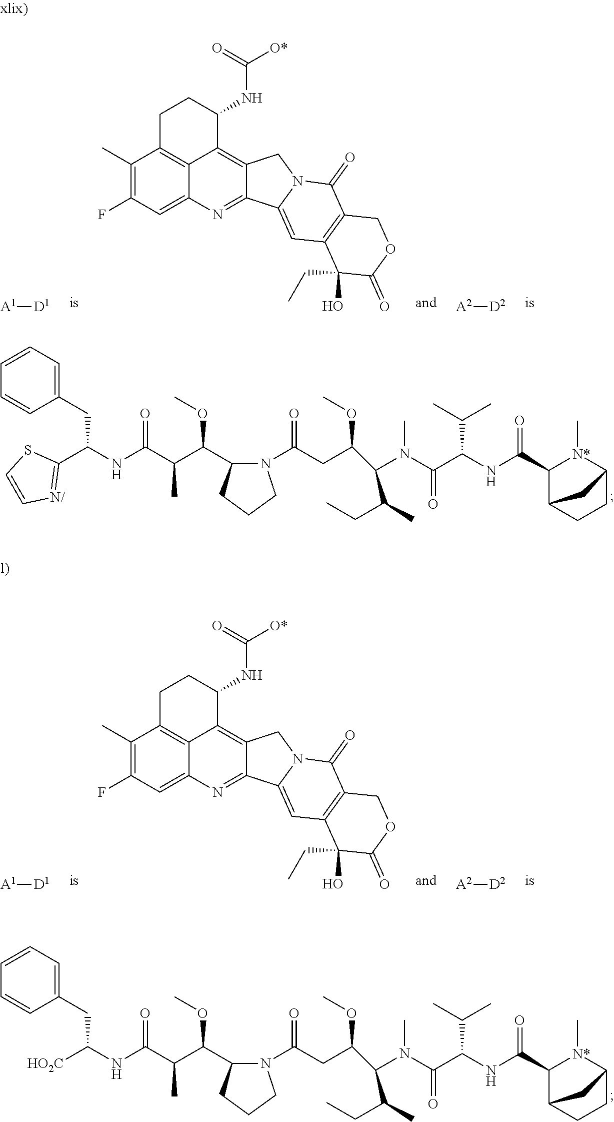

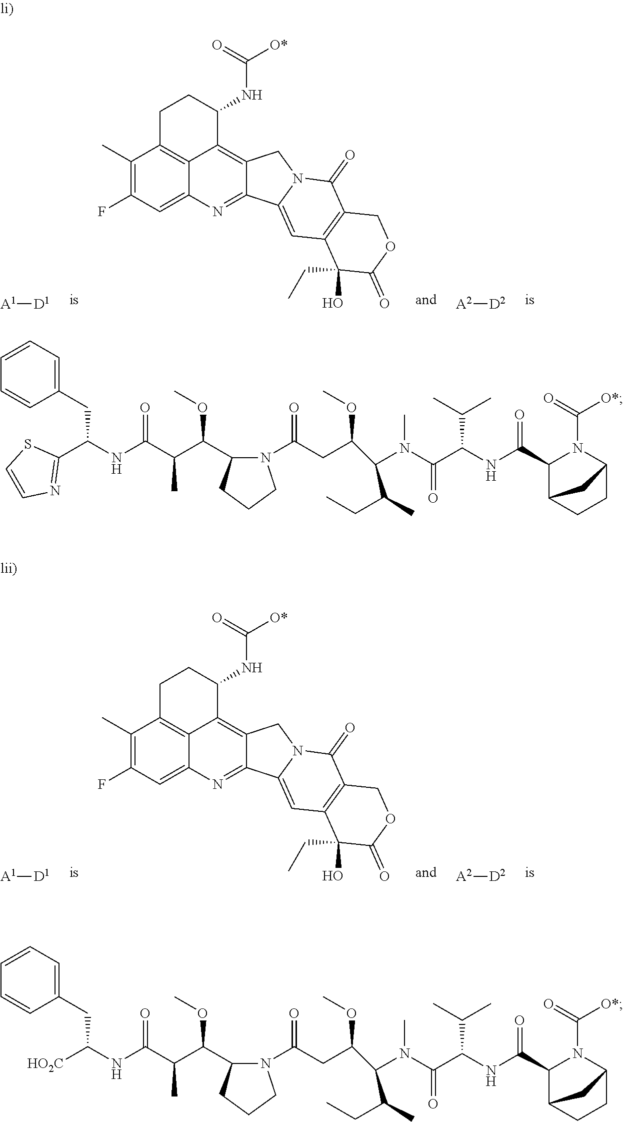

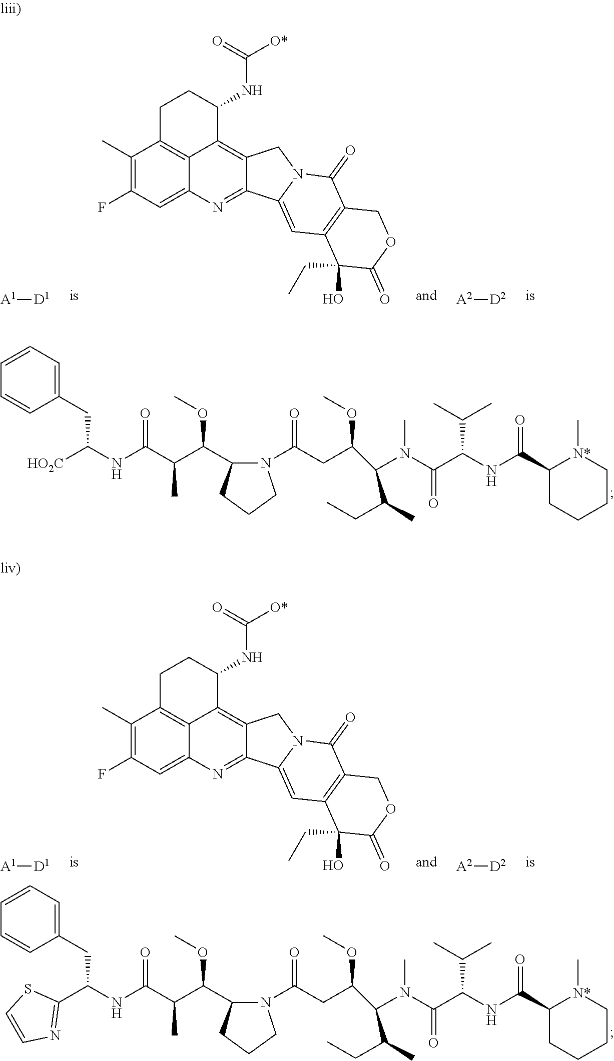

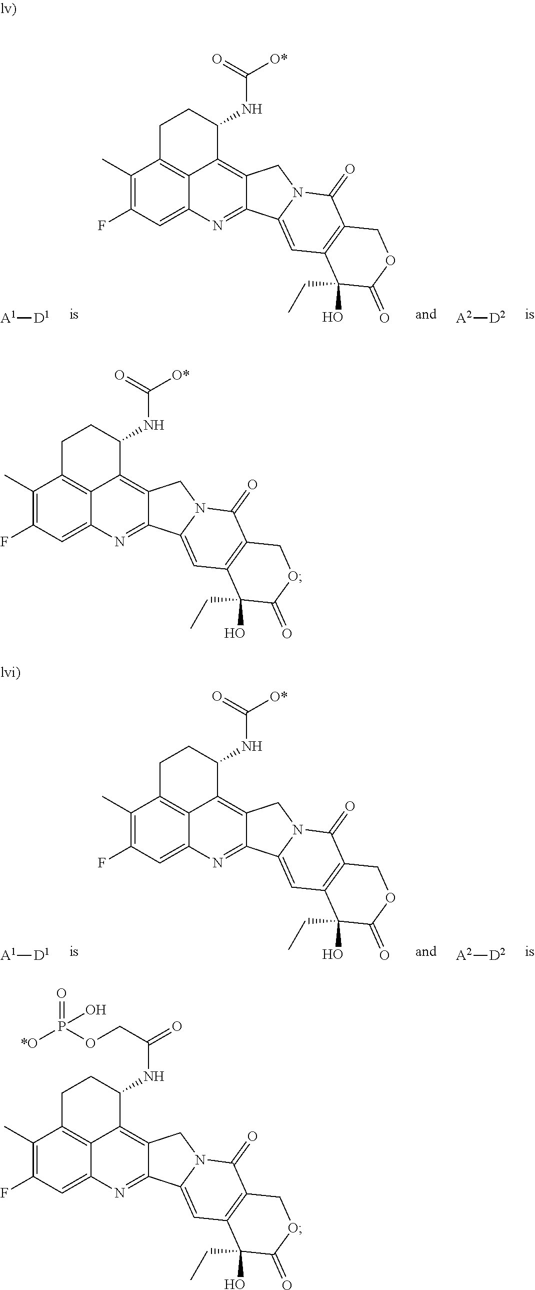

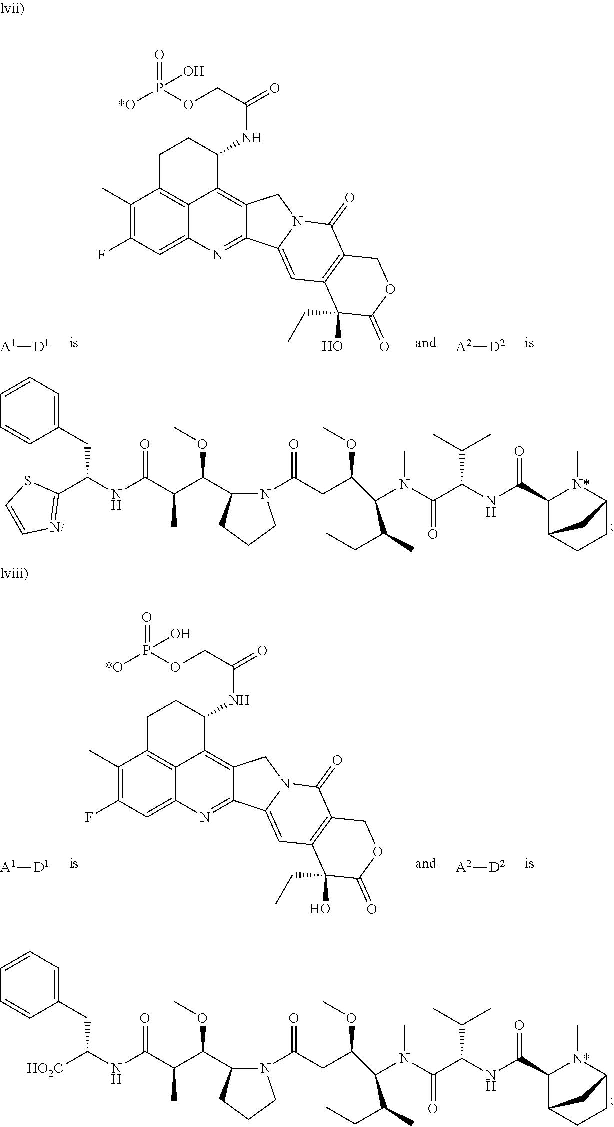

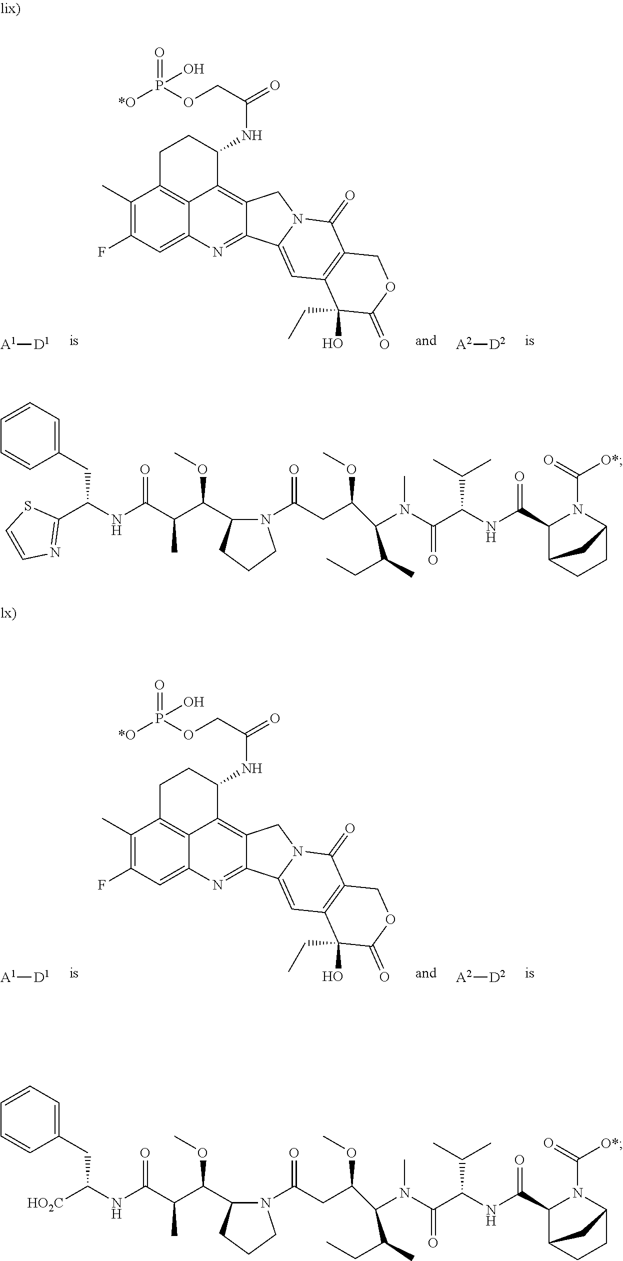

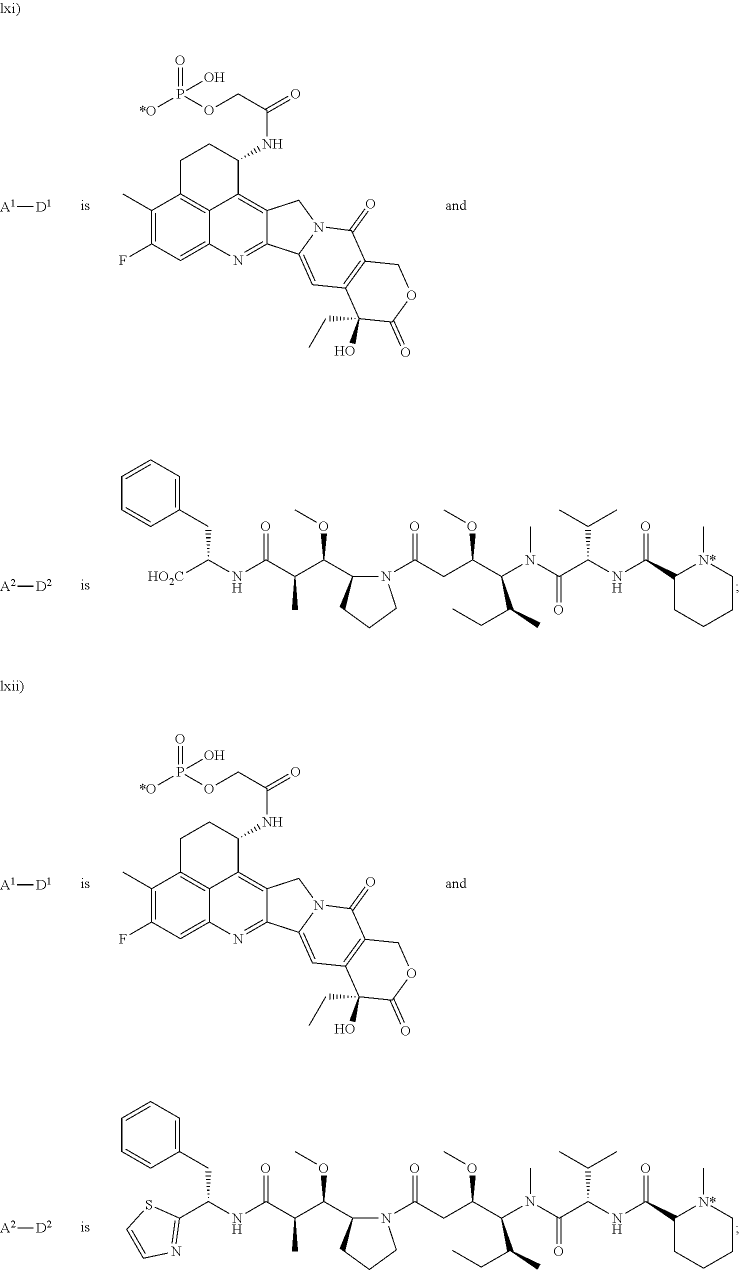

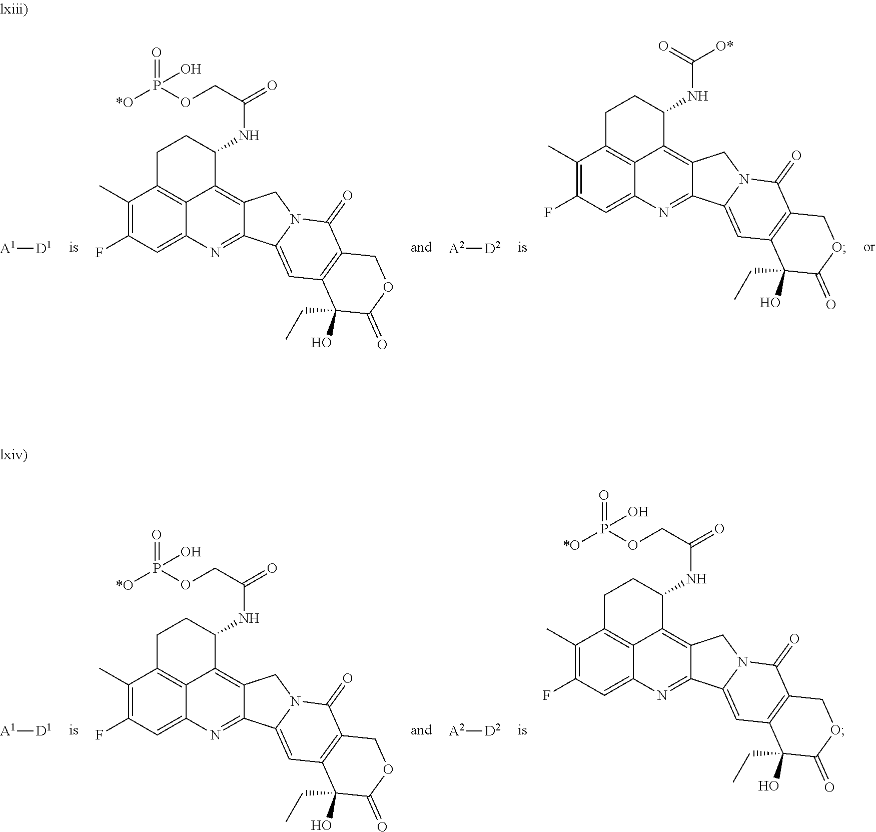

wherein * indicates the point of connection to the remainder of the molecule.
   In an embodiment, D¹ and/or D² are an auristatin.
   In an embodiment, both D¹ and D² are an auristatin.
   In an embodiment, D¹ and/or D² are a topoisomerase 1 inhibitor.
   In an embodiment, both D¹ and D² are a topoisomerase 1 inhibitor.
   In an embodiment, one of D¹ and D² is an auristatin and the other of D¹ and D² is a topoisomerase 1 inhibitor.
   In an embodiment, D¹ is an auristatin and D² is a topoisomerase 1 inhibitor.
   In an embodiment, D¹ is a topoisomerase 1 inhibitor and D² is an auristatin.
   In an embodiment, D¹ and D² are independently selected from the group consisting of

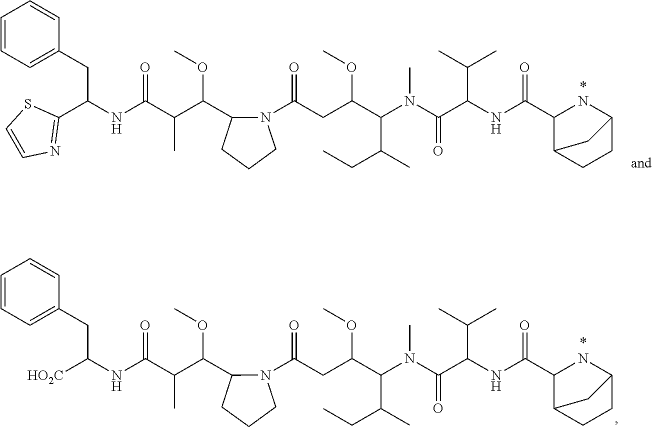

wherein * indicates the point of attachment to A¹ or A²; or a pharmaceutically acceptable salt thereof.

In an embodiment, D¹ and D² are independently selected from the group consisting of

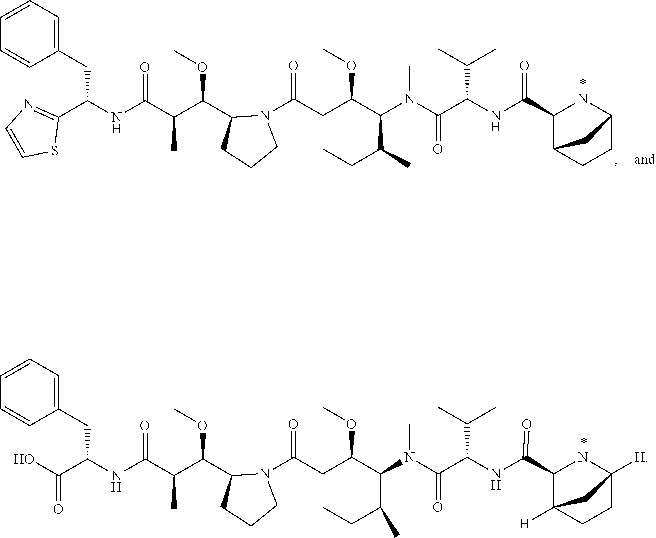

In an embodiment, D¹ and D² are independently selected from the group consisting of:

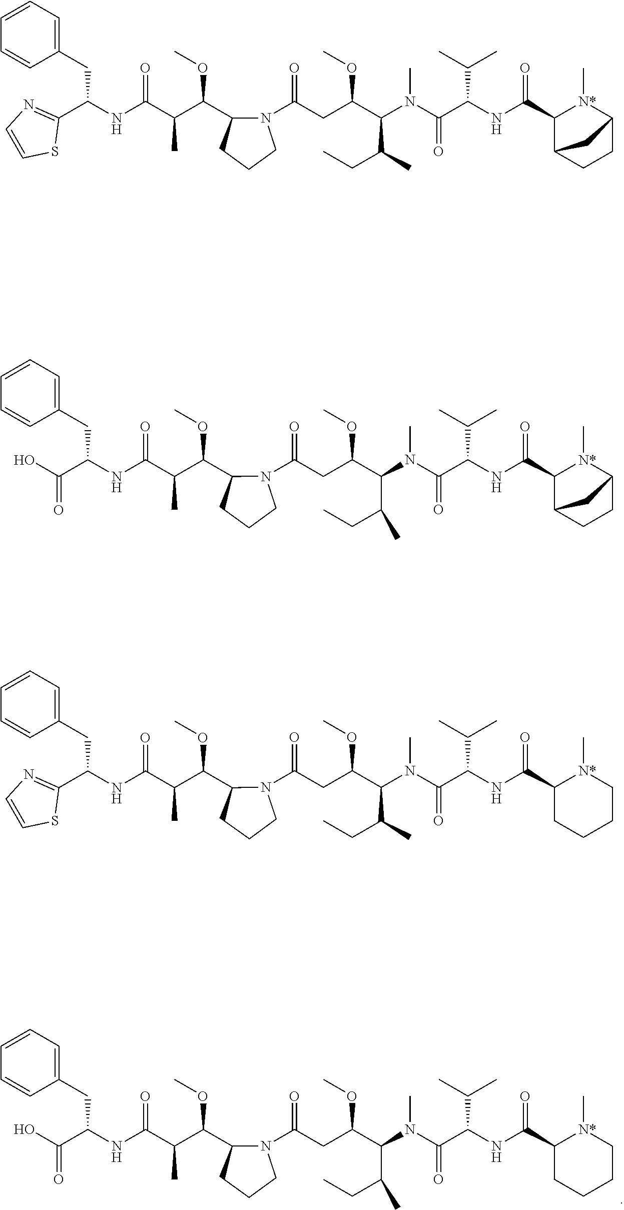

In an embodiment, D1 and D2 are each independently selected from

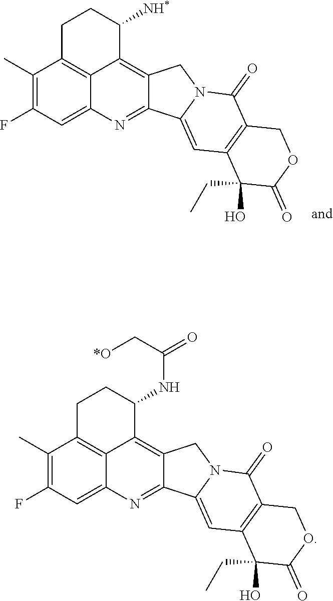

In an embodiment, D¹ and D² are each independently selected from an antitubulin agent, a tubulin inhibitor, a DNA minor groove binder, a DNA replication inhibitor, an alkylating agent, an antibiotic, an antifolate, an antimetabolite, a chemotherapy sensitizer, a topoisomerase inhibitor, and/or a vinca alkaloid.

In an embodiment, D¹ and D² are each independently selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor, a DHFR inhibitor, or a pro-apoptotic agent, optionally with the proviso that neither D¹ nor D² is a BH3 mimetic.

In an embodiment, the compound or pharmaceutically acceptable salt thereof is selected from the group consisting of:

P1-L1-P1, P1-L1-P2, P1-L1-P3, P1-L1-P4, P1-L1-P5, P1-L1-P6, P1-L1-P7, P1-L1-P8, P1-L2-P1, P1-L2-P2, P1-L2-P3, P1-L2-P4, P1-L2-P5, P1-L2-P6, P1-L2-P7, P1-L2-P8, P1-L3-P1, P1-L3-P2, P1-L3-P3, P1-L3-P4, P1-L3-P5, P1-L3-P6, P1-L3-P7, P1-L3-P8, P1-L4-P1, P1-L4-P2, P1-L4-P3, P1-L4-P4, P1-L4-P5, P1-L4-P6, P1-L4-P7, P1-L4-P8, P1-L5-P1, P1-L5-P2, P1-L5-P3, P1-L5-P4, P1-L5-P5, P1-L5-P6, P1-L5-P7, P1-L5-P8, P1-L6-P1, P1-L6-P2, P1-L6-P3, P1-L6-P4, P1-L6-P5, P1-L6-P6, P1-L6-P7, P1-L6-P8, P1-L7-P1, P1-L7-P2, P1-L7-P3, P1-L7-P4, P1-L7-P5, P1-L7-P6, P1-L7-P7, P1-L7-P8, P1-L8-P1, P1-L8-P2, P1-L8-P3, P1-L8-P4, P1-L8-P5, P1-L8-P6, P1-L8-P7, P1-L8-P8, P1-L9-P1, P1-L9-P2, P1-L9-P3, P1-L9-P4, P1-L9-P5, P1-L9-P6, P1-L9-P7, P1-L9-P8, P1-L10-P1, P1-L10-P2, P1-10-P3, P1-L10-P4, P1-10-P5, P1-L10-P6, P1-L10-P7, P1-10-P8, P1-L11-P1, P1-L11-P2, P1-L11-P3, P1-L11-P4, P1-L11-P5, P1-L11-P6, P1-L11-P7, P1-L11-P8, P1-L12-P1, P1-L12-P2, P1-L12-P3, P1-L12-P4, P1-L12-P5, P1-L12-P6, P1-L12-P7, P1-L12-P8, P1-L13-P1, P1-L13-P2, P1-L13-P3, P1-L13-P4, P1-L13-P5, P1-L13-P6, P1-L13-P7, P1-L13-P8, P1-L14-P1, P1-L14-P2, P1-L14-P3, P1-L14-P4, P1-L14-P5, P1-L14-P6, P1-L14-P7, P1-L14-P8, P1-L15-P1, P1-L15-P2, P1-L15-P3, P1-L15-P4, P1-L15-P5, P1-L15-P6, P1-L15-P7, P1-L15-P8, P1-L16-P1, P1-L16-P2, P1-L16-P3, P1-L16-P4, P1-L16-P5, P1-L16-P6, P1-L16-P7, P1-L16-P8, P1-L17-P1, P1-L17-P2, P1-L17-P3, P1-L17-P4, P1-L17-P5, P1-L17-P6, P1-L17-P7, P1-L17-P8, P1-L18-P1, P1-L18-P2, P1-L18-P3, P1-L18-P4, P1-L18-P5, P1-L18-P6, P1-L18-P7, P1-L18-P8, P1-L19-P1, P1-L19-P2, P1-L19-P3, P1-L19-P4, P1-L19-P5, P1-L19-P6, P1-L19-P7, P1-L19-P8, P1-L20-P1, P1-L20-P2, P1-L20-P3, P1-L20-P4, P1-L20-P5, P1-L20-P6, P1-L20-P7, P1-L20-P8, P1-L21-P1, P1-L21-P2, P1-L21-P3, P1-L21-P4, P1-L21-P5, P1-L21-P6, P1-L21-P7, P1-L21-P8, P1-L22-P1, P1-L22-P2, P1-L22-P3, P1-L22-P4, P1-L22-P5, P1-L22-P6, P1-L22-P7, P1-L22-P8, P1-L23-P1, P1-L23-P2, P1-L23-P3, P1-L23-P4, P1-L23-P5, P1-L23-P6, P1-L23-P7, P1-L23-P8, P1-L24-P1, P1-L24-P2, P1-L24-P3, P1-L24-P4, P1-L24-P5, P1-L24-P6, P1-L24-P7, P1-L24-P8, P1-L25-P1, P1-L25-P2, P1-L25-P3, P1-L25-P4, P1-L25-P5, P1-L25-P6, P1-L25-P7, P1-L25-P8, P1-L26-P1, P1-L26-P2, P1-L26-P3, P1-L26-P4, P1-L26-P5, P1-L26-P6, P1-L26-P7, P1-L26-P8, P1-L27-P1, P1-L27-P2, P1-L27-P3, P1-L27-P4, P1-L27-P5, P1-L27-P6, P1-L27-P7, P1-L27-P8, P1-L28-P1, P1-L28-P2, P1-L28-P3, P1-L28-P4, P1-L28-P5, P1-L28-P6, P1-L28-P7, P1-L28-P8, P1-L29-P1, P1-L29-P2, P1-L29-P3, P1-L29-P4, P1-L29-P5, P1-L29-P6, P1-L29-P7, P1-L29-P8, P1-L30-P1, P1-L30-P2, P1-L30-P3, P1-L30-P4, P1-L30-P5, P1-L30-P6, P1-L30-P7, P1-L30-P8, P1-L31-P1, P1-L31-P2, P1-L31-P3, P1-L31-P4, P1-L31-P5, P1-L31-P6, P1-L31-P7, P1-L31-P8, P1-L32-P1, P1-L32-P2, P1-L32-P3, P1-L32-P4, P1-L32-P5, P1-L32-P6, P1-L32-P7, P1-L32-P8, P1-L33-P1, P1-L33-P2, P1-L33-P3, P1-L33-P4, P1-L33-P5, P1-L33-P6, P1-L33-P7, P1-L33-P8, P1-L34-P1, P1-L34-P2, P1-L34-P3, P1-L34-P4, P1-L34-P5, P1-L34-P6, P1-L34-P7, P1-L34-P8, P1-L35-P1, P1-L35-P2, P1-L35-P3, P1-L35-P4, P1-L35-P5, P1-L35-P6, P1-L35-P7, P1-L35-P8, P1-L36-P1, P1-L36-P2, P1-L36-P3, P1-L36-P4, P1-L36-P5, P1-L36-P6, P1-L36-P7, P1-L36-P8, P2-L1-P1, P2-L1-P2, P2-L1-P3, P2-L1-P4, P2-L1-P5, P2-L1-P6, P2-L1-P7, P2-L1-P8, P2-L2-P1, P2-L2-P2, P2-L2-P3, P2-L2-P4, P2-L2-P5, P2-L2-P6, P2-L2-P7, P2-L2-P8, P2-L3-P1, P2-L3-P2, P2-L3-P3, P2-L3-P4, P2-L3-P5, P2-L3-P6, P2-L3-P7, P2-L3-P8, P2-L4-P1, P2-L4-P2, P2-L4-P3, P2-L4-P4, P2-L4-P5, P2-L4-P6, P2-L4-P7, P2-L4-P8, P2-L5-P1, P2-L5-P2, P2-L5-P3, P2-L5-P4, P2-L5-P5, P2-L5-P6, P2-L5-P7, P2-L5-P8, P2-L6-P1, P2-L6-P2, P2-L6-P3, P2-L6-P4, P2-L6-P5, P2-L6-P6, P2-L6-P7, P2-L6-P8, P2-L7-P1, P2-L7-P2, P2-L7-P3, P2-L7-P4, P2-L7-P5, P2-L7-P6, P2-L7-P7, P2-L7-P8, P2-L8-P1, P2-L8-P2, P2-L8-P3, P2-L8-P4, P2-L8-P5, P2-L8-P6, P2-L8-P7, P2-L8-P8, P2-L9-P1, P2-L9-P2, P2-L9-P3, P2-L9-P4, P2-L9-P5, P2-L9-P6, P2-L9-P7, P2-L9-P8, P2-L10-P1, P2-L10-P2, P2-L10-P3, P2-L10-P4, P2-L10-P5, P2-L10-P6, P2-L10-P7, P2-10-P8, P2-L11-P1, P2-L11-P2, P2-L11-P3, P2-L11-P4, P2-L11-P5, P2-L11-P6, P2-L11-P7, P2-L11-P8, P2-L12-P1, P2-L12-P2, P2-L12-P3, P2-L12-P4, P2-L12-P5, P2-L12-P6, P2-L12-P7, P2-L12-P8, P2-L13-P1, P2-L13-P2, P2-L13-P3, P2-L13-P4, P2-L13-P5, P2-L13-P6, P2-L13-P7, P2-L13-P8, P2-L14-P1, P2-L14-P2, P2-L14-P3, P2-L14-P4, P2-L14-P5, P2-L14-P6, P2-L14-P7, P2-L14-P8, P2-L15-P1, P2-L15-P2, P2-L15-P3, P2-L15-P4, P2-L15-P5, P2-L15-P6, P2-L15-P7, P2-L15-P8, P2-L16-P1, P2-L16-P2, P2-L16-P3, P2-L16-P4, P2-L16-P5, P2-L16-P6, P2-L16-P7, P2-L16-P8, P2-L17-P1, P2-L17-P2, P2-L17-P3, P2-L17-P4, P2-L17-P5, P2-L17-P6, P2-L17-P7, P2-L17-P8, P2-L18-P1, P2-L18-P2, P2-L18-P3, P2-L18-P4, P2-L18-P5, P2-L18-P6, P2-L18-P7, P2-L18-P8, P2-L19-P1, P2-L19-P2, P2-L19-P3, P2-L19-P4, P2-L19-P5, P2-L19-P6, P2-L19-P7, P2-L19-P8, P2-L20-P1, P2-L20-P2, P2-L20-P3, P2-L20-P4, P2-L20-P5, P2-L20-P6, P2-L20-P7, P2-L20-P8, P2-L21-P1, P2-L21-P2, P2-L21-P3, P2-L21-P4, P2-L21-P5, P2-L21-P6, P2-L21-P7, P2-L21-P8, P2-L22-P1, P2-L22-P2, P2-L22-P3, P2-L22-P4, P2-L22-P5, P2-L22-P6, P2-L22-P7, P2-L22-P8, P2-L23-P1, P2-L23-P2, P2-L23-P3, P2-L23-P4, P2-L23-P5, P2-L23-P6, P2-L23-P7, P2-L23-P8, P2-L24-P1, P2-L24-P2, P2-L24-P3, P2-L24-P4, P2-L24-P5, P2-L24-P6, P2-L24-P7, P2-L24-P8, P2-L25-P1, P2-L25-P2, P2-L25-P3, P2-L25-P4, P2-L25-P5, P2-L25-P6, P2-L25-P7, P2-L25-P8, P2-L26-P1, P2-L26-P2, P2-L26-P3, P2-L26-P4, P2-L26-P5, P2-L26-P6, P2-L26-P7, P2-L26-P8, P2-L27-P1, P2-L27-P2, P2-L27-P3, P2-L27-P4, P2-L27-P5, P2-L27-P6, P2-L27-P7, P2-L27-P8, P2-L28-P1, P2-L28-P2, P2-L28-P3, P2-L28-P4, P2-L28-P5, P2-L28-P6, P2-L28-P7, P2-L28-P8, P2-L29-P1, P2-L29-P2, P2-L29-P3, P2-L29-P4, P2-L29-P5, P2-L29-P6, P2-L29-P7, P2-L29-P8, P2-L30-P1, P2-L30-P2, P2-L30-P3, P2-L30-P4, P2-L30-P5, P2-L30-P6, P2-L30-P7, P2-L30-P8, P2-L31-P1, P2-L31-P2, P2-L31-P3, P2-L31-P4, P2-L31-P5, P2-L31-P6, P2-L31-P7, P2-L31-P8, P2-L32-P1, P2-L32-P2, P2-L32-P3, P2-L32-P4, P2-L32-P5, P2-L32-P6, P2-L32-P7, P2-L32-P8, P2-L33-P1, P2-L33-P2, P2-L33-P3, P2-L33-P4, P2-L33-P5, P2-L33-P6, P2-L33-P7, P2-L33-P8, P2-L34-P1, P2-L34-P2, P2-L34-P3, P2-L34-P4, P2-L34-P5, P2-L34-P6, P2-L34-P7, P2-L34-P8, P2-L35-P1, P2-L35-P2, P2-L35-P3, P2-L35-P4, P2-L35-P5, P2-L35-P6, P2-L35-P7, P2-L35-P8, P2-L36-P1, P2-L36-P2, P2-L36-P3, P2-L36-P4, P2-L36-P5, P2-L36-P6, P2-L36-P7, P2-L36-P8, P3-L1-P1, P3-L1-P2, P3-L1-P3, P3-L1-P4, P3-L1-P5, P3-L1-P6, P3-L1-P7, P3-L1-P8, P3-L2-P1, P3-L2-P2, P3-L2-P3, P3-L2-P4, P3-L2-P5, P3-L2-P6, P3-L2-P7, P3-L2-P8, P3-L3-P1, P3-L3-P2, P3-L3-P3, P3-L3-P4, P3-L3-P5, P3-L3-P6, P3-L3-P7, P3-L3-P8, P3-L4-P1, P3-L4-P2, P3-L4-P3, P3-L4-P4, P3-L4-P5, P3-L4-P6, P3-L4-P7, P3-L4-P8, P3-L5-P1, P3-L5-P2, P3-L5-P3, P3-L5-P4, P3-L5-P5, P3-L5-P6, P3-L5-P7, P3-L5-P8, P3-L6-P1, P3-L6-P2, P3-L6-P3, P3-L6-P4, P3-L6-P5, P3-L6-P6, P3-L6-P7, P3-L6-P8, P3-L7-P1, P3-L7-P2, P3-L7-P3, P3-L7-P4, P3-L7-P5, P3-L7-P6, P3-L7-P7, P3-L7-P8, P3-L8-P1, P3-L8-P2, P3-L8-P3, P3-L8-P4, P3-L8-P5, P3-L8-P6, P3-L8-P7, P3-L8-P8, P3-L9-P1, P3-L9-P2, P3-L9-P3, P3-L9-P4, P3-L9-P5, P3-L9-P6, P3-L9-P7, P3-L9-P8, P3-L10-P1, P3-L10-P2, P3-L10-P3, P3-L10-P4, P3-L10-P5, P3-L10-P6, P3-L10-P7, P3-L10-P8, P3-11-P1, P3-L11-P2, P3-L11-P3, P3-L11-P4, P3-L11-P5, P3-L11-P6, P3-L11-P7, P3-L11-P8, P3-L12-P1, P3-L12-P2, P3-L12-P3, P3-L12-P4, P3-L12-P5, P3-L12-P6, P3-L12-P7, P3-L12-P8, P3-L13-P1, P3-L13-P2, P3-L13-P3, P3-L13-P4, P3-L13-P5, P3-L13-P6, P3-L13-P7, P3-L13-P8, P3-L14-P1, P3-L14-P2, P3-L14-P3, P3-L14-P4, P3-L14-P5, P3-L14-P6, P3-L14-P7, P3-L14-P8, P3-L15-P1, P3-L15-P2, P3-L15-P3, P3-L15-P4, P3-L15-P5, P3-L15-P6, P3-L15-P7, P3-L15-P8, P3-L16-P1, P3-L16-P2, P3-L16-P3, P3-L16-P4, P3-L16-P5, P3-L16-P6, P3-L16-P7, P3-L16-P8, P3-L17-P1, P3-L17-P2, P3-L17-P3, P3-L17-P4, P3-L17-P5, P3-L17-P6, P3-L17-P7, P3-L17-P8, P3-L18-P1, P3-L18-P2, P3-L18-P3, P3-L18-P4, P3-L18-P5, P3-L18-P6, P3-L18-P7, P3-L18-P8, P3-L19-P1, P3-L19-P2, P3-L19-P3, P3-L19-P4, P3-L19-P5, P3-L19-P6, P3-L19-P7, P3-L19-P8, P3-L20-P1, P3-L20-P2, P3-L20-P3, P3-L20-P4, P3-L20-P5, P3-L20-P6, P3-L20-P7, P3-L20-P8, P3-L21-P1, P3-L21-P2, P3-L21-P3, P3-L21-P4, P3-L21-P5, P3-L21-P6, P3-L21-P7, P3-L21-P8, P3-L22-P1, P3-L22-P2, P3-L22-P3, P3-L22-P4, P3-L22-P5, P3-L22-P6, P3-L22-P7, P3-L22-P8, P3-L23-P1, P3-L23-P2, P3-L23-P3, P3-L23-P4, P3-L23-P5, P3-L23-P6, P3-L23-P7, P3-L23-P8, P3-L24-P1, P3-L24-P2, P3-L24-P3, P3-L24-P4, P3-L24-P5, P3-L24-P6, P3-L24-P7, P3-L24-P8, P3-L25-P1, P3-L25-P2, P3-L25-P3, P3-L25-P4, P3-L25-P5, P3-L25-P6, P3-L25-P7, P3-L25-P8, P3-L26-P1, P3-L26-P2, P3-L26-P3, P3-L26-P4, P3-L26-P5, P3-L26-P6, P3-L26-P7, P3-L26-P8, P3-L27-P1, P3-L27-P2, P3-L27-P3, P3-L27-P4, P3-L27-P5, P3-L27-P6, P3-L27-P7, P3-L27-P8, P3-L28-P1, P3-L28-P2, P3-L28-P3, P3-L28-P4, P3-L28-P5, P3-L28-P6, P3-L28-P7, P3-L28-P8, P3-L29-P1, P3-L29-P2, P3-L29-P3, P3-L29-P4, P3-L29-P5, P3-L29-P6, P3-L29-P7, P3-L29-P8, P3-L30-P1, P3-L30-P2, P3-L30-P3, P3-L30-P4, P3-L30-P5, P3-L30-P6, P3-L30-P7, P3-L30-P8, P3-L31-P1, P3-L31-P2, P3-L31-P3, P3-L31-P4, P3-L31-P5, P3-L31-P6, P3-L31-P7, P3-L31-P8, P3-L32-P1, P3-L32-P2, P3-L32-P3, P3-L32-P4, P3-L32-P5, P3-L32-P6, P3-L32-P7, P3-L32-P8, P3-L33-P1, P3-L33-P2, P3-L33-P3, P3-L33-P4, P3-L33-P5, P3-L33-P6, P3-L33-P7, P3-L33-P8, P3-L34-P1, P3-L34-P2, P3-L34-P3, P3-L34-P4, P3-L34-P5, P3-L34-P6, P3-L34-P7, P3-L34-P8, P3-L35-P1, P3-L35-P2, P3-L35-P3, P3-L35-P4, P3-L35-P5, P3-L35-P6, P3-L35-P7, P3-L35-P8, P3-L36-P1, P3-L36-P2, P3-L36-P3, P3-L36-P4, P3-L36-P5, P3-L36-P6, P3-L36-P7, P3-L36-P8, P4-L1-P1, P4-L1-P2, P4-L1-P3, P4-L1-P4, P4-L1-P5, P4-L1-P6, P4-L1-P7, P4-L1-P8, P4-L2-P1, P4-L2-P2, P4-L2-P3, P4-L2-P4, P4-L2-P5, P4-L2-P6, P4-L2-P7, P4-L2-P8, P4-L3-P1, P4-L3-P2, P4-L3-P3, P4-L3-P4, P4-L3-P5, P4-L3-P6, P4-L3-P7, P4-L3-P8, P4-L4-P1, P4-L4-P2, P4-L4-P3, P4-L4-P4, P4-L4-P5, P4-L4-P6, P4-L4-P7, P4-L4-P8, P4-L5-P1, P4-L5-P2, P4-L5-P3, P4-L5-P4, P4-L5-P5, P4-L5-P6, P4-L5-P7, P4-L5-P8, P4-L6-P1, P4-L6-P2, P4-L6-P3, P4-L6-P4, P4-L6-P5, P4-L6-P6, P4-L6-P7, P4-L6-P8, P4-L7-P1, P4-L7-P2, P4-L7-P3, P4-L7-P4, P4-L7-P5, P4-L7-P6, P4-L7-P7, P4-L7-P8, P4-L8-P1, P4-L8-P2, P4-L8-P3, P4-L8-P4, P4-L8-P5, P4-L8-P6, P4-L8-P7, P4-L8-P8, P4-L9-P1, P4-L9-P2, P4-L9-P3, P4-L9-P4, P4-L9-P5, P4-L9-P6, P4-L9-P7, P4-L9-P8, P4-10-P1, P4-10-P2, P4-10-P3, P4-L10-P4, P4-L10-P5, P4-10-P6, P4-10-P7, P4-10-P8, P4-11-P1, P4-L11-P2, P4-L11-P3, P4-L11-P4, P4-L11-P5, P4-L11-P6, P4-L11-P7, P4-L11-P8, P4-L12-P1, P4-L12-P2, P4-L12-P3, P4-L12-P4, P4-L12-P5, P4-L12-P6, P4-L12-P7, P4-L12-P8, P4-L13-P1, P4-L13-P2, P4-L13-P3, P4-L13-P4, P4-L13-P5, P4-L13-P6, P4-L13-P7, P4-L13-P8, P4-L14-P1, P4-L14-P2, P4-L14-P3, P4-L14-P4, P4-L14-P5, P4-L14-P6, P4-L14-P7, P4-L14-P8, P4-L15-P1, P4-L15-P2, P4-L15-P3, P4-L15-P4, P4-L15-P5, P4-L15-P6, P4-L15-P7, P4-L15-P8, P4-L16-P1, P4-L16-P2, P4-L16-P3, P4-L16-P4, P4-L16-P5, P4-L16-P6, P4-L16-P7, P4-L16-P8, P4-L17-P1, P4-L17-P2, P4-L17-P3, P4-L17-P4, P4-L17-P5, P4-L17-P6, P4-L17-P7, P4-L17-P8, P4-L18-P1, P4-L18-P2, P4-L18-P3, P4-L18-P4, P4-L18-P5, P4-L18-P6, P4-L18-P7, P4-L18-P8, P4-L19-P1, P4-L19-P2, P4-L19-P3, P4-L19-P4, P4-L19-P5, P4-L19-P6, P4-L19-P7, P4-L19-P8, P4-L20-P1, P4-L20-P2, P4-L20-P3, P4-L20-P4, P4-L20-P5, P4-L20-P6, P4-L20-P7, P4-L20-P8, P4-L21-P1, P4-L21-P2, P4-L21-P3, P4-L21-P4, P4-L21-P5, P4-L21-P6, P4-L21-P7, P4-L21-P8, P4-L22-P1, P4-L22-P2, P4-L22-P3, P4-L22-P4, P4-L22-P5, P4-L22-P6, P4-L22-P7, P4-L22-P8, P4-L23-P1, P4-L23-P2, P4-L23-P3, P4-L23-P4, P4-L23-P5, P4-L23-P6, P4-L23-P7, P4-L23-P8, P4-L24-P1, P4-L24-P2, P4-L24-P3, P4-L24-P4, P4-L24-P5, P4-L24-P6, P4-L24-P7, P4-L24-P8, P4-L25-P1, P4-L25-P2, P4-L25-P3, P4-L25-P4, P4-L25-P5, P4-L25-P6, P4-L25-P7, P4-L25-P8, P4-L26-P1, P4-L26-P2, P4-L26-P3, P4-L26-P4, P4-L26-P5, P4-L26-P6, P4-L26-P7, P4-L26-P8, P4-L27-P1, P4-L27-P2, P4-L27-P3, P4-L27-P4, P4-L27-P5, P4-L27-P6, P4-L27-P7, P4-L27-P8, P4-L28-P1, P4-L28-P2, P4-L28-P3, P4-L28-P4, P4-L28-P5, P4-L28-P6, P4-L28-P7, P4-L28-P8, P4-L29-P1, P4-L29-P2, P4-L29-P3, P4-L29-P4, P4-L29-P5, P4-L29-P6, P4-L29-P7, P4-L29-P8, P4-L30-P1, P4-L30-P2, P4-L30-P3, P4-L30-P4, P4-L30-P5, P4-L30-P6, P4-L30-P7, P4-L30-P8, P4-L31-P1, P4-L31-P2, P4-L31-P3, P4-L31-P4, P4-L31-P5, P4-L31-P6, P4-L31-P7, P4-L31-P8, P4-L32-P1, P4-L32-P2, P4-L32-P3, P4-L32-P4, P4-L32-P5, P4-L32-P6, P4-L32-P7, P4-L32-P8, P4-L33-P1, P4-L33-P2, P4-L33-P3, P4-L33-P4, P4-L33-P5, P4-L33-P6, P4-L33-P7, P4-L33-P8, P4-L34-P1, P4-L34-P2, P4-L34-P3, P4-L34-P4, P4-L34-P5, P4-L34-P6, P4-L34-P7, P4-L34-P8, P4-L35-P1, P4-L35-P2, P4-L35-P3, P4-L35-P4, P4-L35-P5, P4-L35-P6, P4-L35-P7, P4-L35-P8, P4-L36-P1, P4-L36-P2, P4-L36-P3, P4-L36-P4, P4-L36-P5, P4-L36-P6, P4-L36-P7, P4-L36-P8, P5-L1-P1, P5-L1-P2, P5-L1-P3, P5-L1-P4, P5-L1-P5, P5-L1-P6, P5-L1-P7, P5-L1-P8, P5-L2-P1, P5-L2-P2, P5-L2-P3, P5-L2-P4, P5-L2-P5, P5-L2-P6, P5-L2-P7, P5-L2-P8, P5-L3-P1, P5-L3-P2, P5-L3-P3, P5-L3-P4, P5-L3-P5, P5-L3-P6, P5-L3-P7, P5-L3-P8, P5-L4-P1, P5-L4-P2, P5-L4-P3, P5-L4-P4, P5-L4-P5, P5-L4-P6, P5-L4-P7, P5-L4-P8, P5-L5-P1, P5-L5-P2, P5-L5-P3, P5-L5-P4, P5-L5-P5, P5-L5-P6, P5-L5-P7, P5-L5-P8, P5-L6-P1, P5-L6-P2, P5-L6-P3, P5-L6-P4, P5-L6-P5, P5-L6-P6, P5-L6-P7, P5-L6-P8, P5-L7-P1, P5-L7-P2, P5-L7-P3, P5-L7-P4, P5-L7-P5, P5-L7-P6, P5-L7-P7, P5-L7-P8, P5-L8-P1, P5-L8-P2, P5-L8-P3, P5-L8-P4, P5-L8-P5, P5-L8-P6, P5-L8-P7, P5-L8-P8, P5-L9-P1, P5-L9-P2, P5-L9-P3, P5-L9-P4, P5-L9-P5, P5-L9-P6, P5-L9-P7, P5-L9-P8, P5-10-P1, P5-110-P2, P5-10-P3, P5-L10-P4, P5-10-P5, P5-10-P6, P5-10-P7, P5-10-P8, P5-L11-P1, P5-L11-P2, P5-L11-P3, P5-L11-P4, P5-L11-P5, P5-L11-P6, P5-L11-P7, P5-L11-P8, P5-L12-P1, P5-L12-P2, P5-L12-P3, P5-L12-P4, P5-L12-P5, P5-L12-P6, P5-L12-P7, P5-L12-P8, P5-L13-P1, P5-L13-P2, P5-L13-P3, P5-L13-P4, P5-L13-P5, P5-L13-P6, P5-L13-P7, P5-L13-P8, P5-L14-P1, P5-L14-P2, P5-L14-P3, P5-L14-P4, P5-L14-P5, P5-L14-P6, P5-L14-P7, P5-L14-P8, P5-L15-P1, P5-L15-P2, P5-L15-P3, P5-L15-P4, P5-L15-P5, P5-L15-P6, P5-L15-P7, P5-L15-P8, P5-L16-P1, P5-L16-P2, P5-L16-P3, P5-L16-P4, P5-L16-P5, P5-L16-P6, P5-L16-P7, P5-L16-P8, P5-L17-P1, P5-L17-P2, P5-L17-P3, P5-L17-P4, P5-L17-P5, P5-L17-P6, P5-L17-P7, P5-L17-P8, P5-L18-P1, P5-L18-P2, P5-L18-P3, P5-L18-P4, P5-L18-P5, P5-L18-P6, P5-L18-P7, P5-L18-P8, P5-L19-P1, P5-L19-P2, P5-L19-P3, P5-L19-P4, P5-L19-P5, P5-L19-P6, P5-L19-P7, P5-L19-P8, P5-L20-P1, P5-L20-P2, P5-L20-P3, P5-L20-P4, P5-L20-P5, P5-L20-P6, P5-L20-P7, P5-L20-P8, P5-L21-P1, P5-L21-P2, P5-L21-P3, P5-L21-P4, P5-L21-P5, P5-L21-P6, P5-L21-P7, P5-L21-P8, P5-L22-P1, P5-L22-P2, P5-L22-P3, P5-L22-P4, P5-L22-P5, P5-L22-P6, P5-L22-P7, P5-L22-P8, P5-L23-P1, P5-L23-P2, P5-L23-P3, P5-L23-P4, P5-L23-P5, P5-L23-P6, P5-L23-P7, P5-L23-P8, P5-L24-P1, P5-L24-P2, P5-L24-P3, P5-L24-P4, P5-L24-P5, P5-L24-P6, P5-L24-P7, P5-L24-P8, P5-L25-P1, P5-L25-P2, P5-L25-P3, P5-L25-P4, P5-L25-P5, P5-L25-P6, P5-L25-P7, P5-L25-P8, P5-L26-P1, P5-L26-P2, P5-L26-P3, P5-L26-P4, P5-L26-P5, P5-L26-P6, P5-L26-P7, P5-L26-P8, P5-L27-P1, P5-L27-P2, P5-L27-P3, P5-L27-P4, P5-L27-P5, P5-L27-P6, P5-L27-P7, P5-L27-P8, P5-L28-P1, P5-L28-P2, P5-L28-P3, P5-L28-P4, P5-L28-P5, P5-L28-P6, P5-L28-P7, P5-L28-P8, P5-L29-P1, P5-L29-P2, P5-L29-P3, P5-L29-P4, P5-L29-P5, P5-L29-P6, P5-L29-P7, P5-L29-P8, P5-L30-P1, P5-L30-P2, P5-L30-P3, P5-L30-P4, P5-L30-P5, P5-L30-P6, P5-L30-P7, P5-L30-P8, P5-L31-P1, P5-L31-P2, P5-L31-P3, P5-L31-P4, P5-L31-P5, P5-L31-P6, P5-L31-P7, P5-L31-P8, P5-L32-P1, P5-L32-P2, P5-L32-P3, P5-L32-P4, P5-L32-P5, P5-L32-P6, P5-L32-P7, P5-L32-P8, P5-L33-P1, P5-L33-P2, P5-L33-P3, P5-L33-P4, P5-L33-P5, P5-L33-P6, P5-L33-P7, P5-L33-P8, P5-L34-P1, P5-L34-P2, P5-L34-P3, P5-L34-P4, P5-L34-P5, P5-L34-P6, P5-L34-P7, P5-L34-P8, P5-L35-P1, P5-L35-P2, P5-L35-P3, P5-L35-P4, P5-L35-P5, P5-L35-P6, P5-L35-P7, P5-L35-P8, P5-L36-P1, P5-L36-P2, P5-L36-P3, P5-L36-P4, P5-L36-P5, P5-L36-P6, P5-L36-P7, P5-L36-P8, P6-L1-P1, P6-L1-P2, P6-L1-P3, P6-L1-P4, P6-L1-P5, P6-L1-P6, P6-L1-P7, P6-L1-P8, P6-L2-P1, P6-L2-P2, P6-L2-P3, P6-L2-P4, P6-L2-P5, P6-L2-P6, P6-L2-P7, P6-L2-P8, P6-L3-P1, P6-L3-P2, P6-L3-P3, P6-L3-P4, P6-L3-P5, P6-L3-P6, P6-L3-P7, P6-L3-P8, P6-L4-P1, P6-L4-P2, P6-L4-P3, P6-L4-P4, P6-L4-P5, P6-L4-P6, P6-L4-P7, P6-L4-P8, P6-L5-P1, P6-L5-P2, P6-L5-P3, P6-L5-P4, P6-L5-P5, P6-L5-P6, P6-L5-P7, P6-L5-P8, P6-L6-P1, P6-L6-P2, P6-L6-P3, P6-L6-P4, P6-L6-P5, P6-L6-P6, P6-L6-P7, P6-L6-P8, P6-L7-P1, P6-L7-P2, P6-L7-P3, P6-L7-P4, P6-L7-P5, P6-L7-P6, P6-L7-P7, P6-L7-P8, P6-L8-P1, P6-L8-P2, P6-L8-P3, P6-L8-P4, P6-L8-P5, P6-L8-P6, P6-L8-P7, P6-L8-P8, P6-L9-P1, P6-L9-P2, P6-L9-P3, P6-L9-P4, P6-L9-P5, P6-L9-P6, P6-L9-P7, P6-L9-P8, P6-L10-P1, P6-L10-P2, P6-L10-P3, P6-L10-P4, P6-L10-P5, P6-L10-P6, P6-L10-P7, P6-L10-P8, P6-11-P1, P6-L11-P2, P6-L11-P3, P6-L11-P4, P6-L11-P5, P6-L11-P6, P6-L11-P7, P6-L11-P8, P6-L12-P1, P6-L12-P2, P6-L12-P3, P6-L12-P4, P6-L12-P5, P6-L12-P6, P6-L12-P7, P6-L12-P8, P6-L13-P1, P6-L13-P2, P6-L13-P3, P6-L13-P4, P6-L13-P5, P6-L13-P6, P6-L13-P7, P6-L13-P8, P6-L14-P1, P6-L14-P2, P6-L14-P3, P6-L14-P4, P6-L14-P5, P6-L14-P6, P6-L14-P7, P6-L14-P8, P6-L15-P1, P6-L15-P2, P6-L15-P3, P6-L15-P4, P6-L15-P5, P6-L15-P6, P6-L15-P7, P6-L15-P8, P6-L16-P1, P6-L16-P2, P6-L16-P3, P6-L16-P4, P6-L16-P5, P6-L16-P6, P6-L16-P7, P6-L16-P8, P6-L17-P1, P6-L17-P2, P6-L17-P3, P6-L17-P4, P6-L17-P5, P6-L17-P6, P6-L17-P7, P6-L17-P8, P6-L18-P1, P6-L18-P2, P6-L18-P3, P6-L18-P4, P6-L18-P5, P6-L18-P6, P6-L18-P7, P6-L18-P8, P6-L19-P1, P6-L19-P2, P6-L19-P3, P6-L19-P4, P6-L19-P5, P6-L19-P6, P6-L19-P7, P6-L19-P8, P6-L20-P1, P6-L20-P2, P6-L20-P3, P6-L20-P4, P6-L20-P5, P6-L20-P6, P6-L20-P7, P6-L20-P8, P6-L21-P1, P6-L21-P2, P6-L21-P3, P6-L21-P4, P6-L21-P5, P6-L21-P6, P6-L21-P7, P6-L21-P8, P6-L22-P1, P6-L22-P2, P6-L22-P3, P6-L22-P4, P6-L22-P5, P6-L22-P6, P6-L22-P7, P6-L22-P8, P6-L23-P1, P6-L23-P2, P6-L23-P3, P6-L23-P4, P6-L23-P5, P6-L23-P6, P6-L23-P7, P6-L23-P8, P6-L24-P1, P6-L24-P2, P6-L24-P3, P6-L24-P4, P6-L24-P5, P6-L24-P6, P6-L24-P7, P6-L24-P8, P6-L25-P1, P6-L25-P2, P6-L25-P3, P6-L25-P4, P6-L25-P5, P6-L25-P6, P6-L25-P7, P6-L25-P8, P6-L26-P1, P6-L26-P2, P6-L26-P3, P6-L26-P4, P6-L26-P5, P6-L26-P6, P6-L26-P7, P6-L26-P8, P6-L27-P1, P6-L27-P2, P6-L27-P3, P6-L27-P4, P6-L27-P5, P6-L27-P6, P6-L27-P7, P6-L27-P8, P6-L28-P1, P6-L28-P2, P6-L28-P3, P6-L28-P4, P6-L28-P5, P6-L28-P6, P6-L28-P7, P6-L28-P8, P6-L29-P1, P6-L29-P2, P6-L29-P3, P6-L29-P4, P6-L29-P5, P6-L29-P6, P6-L29-P7, P6-L29-P8, P6-L30-P1, P6-L30-P2, P6-L30-P3, P6-L30-P4, P6-L30-P5, P6-L30-P6, P6-L30-P7, P6-L30-P8, P6-L31-P1, P6-L31-P2, P6-L31-P3, P6-L31-P4, P6-L31-P5, P6-L31-P6, P6-L31-P7, P6-L31-P8, P6-L32-P1, P6-L32-P2, P6-L32-P3, P6-L32-P4, P6-L32-P5, P6-L32-P6, P6-L32-P7, P6-L32-P8, P6-L33-P1, P6-L33-P2, P6-L33-P3, P6-L33-P4, P6-L33-P5, P6-L33-P6, P6-L33-P7, P6-L33-P8, P6-L34-P1, P6-L34-P2, P6-L34-P3, P6-L34-P4, P6-L34-P5, P6-L34-P6, P6-L34-P7, P6-L34-P8, P6-L35-P1, P6-L35-P2, P6-L35-P3, P6-L35-P4, P6-L35-P5, P6-L35-P6, P6-L35-P7, P6-L35-P8, P6-L36-P1, P6-L36-P2, P6-L36-P3, P6-L36-P4, P6-L36-P5, P6-L36-P6, P6-L36-P7, P6-L36-P8, P7-L1-P1, P7-L1-P2, P7-L1-P3, P7-L1-P4, P7-L1-P5, P7-L1-P6, P7-L1-P7, P7-L1-P8, P7-L2-P1, P7-L2-P2, P7-L2-P3, P7-L2-P4, P7-L2-P5, P7-L2-P6, P7-L2-P7, P7-L2-P8, P7-L3-P1, P7-L3-P2, P7-L3-P3, P7-L3-P4, P7-L3-P5, P7-L3-P6, P7-L3-P7, P7-L3-P8, P7-L4-P1, P7-L4-P2, P7-L4-P3, P7-L4-P4, P7-L4-P5, P7-L4-P6, P7-L4-P7, P7-L4-P8, P7-L5-P1, P7-L5-P2, P7-L5-P3, P7-L5-P4, P7-L5-P5, P7-L5-P6, P7-L5-P7, P7-L5-P8, P7-L6-P1, P7-L6-P2, P7-L6-P3, P7-L6-P4, P7-L6-P5, P7-L6-P6, P7-L6-P7, P7-L6-P8, P7-L7-P1, P7-L7-P2, P7-L7-P3, P7-L7-P4, P7-L7-P5, P7-L7-P6, P7-L7-P7, P7-L7-P8, P7-L8-P1, P7-L8-P2, P7-L8-P3, P7-L8-P4, P7-L8-P5, P7-L8-P6, P7-L8-P7, P7-L8-P8, P7-L9-P1, P7-L9-P2, P7-L9-P3, P7-L9-P4, P7-L9-P5, P7-L9-P6, P7-L9-P7, P7-L9-P8, P7-L10-P1, P7-L10-P2, P7-L10-P3, P7- L10-P4, P7-10-P5, P7-10-P6, P7-10-P7, P7-10-P8, P7-L11-P1, P7-L11-P2, P7-L11-P3, P7-L11-P4, P7-L11-P5, P7-L11-P6, P7-L11-P7, P7-L11-P8, P7-L12-P1, P7-L12-P2, P7-L12-P3, P7-L12-P4, P7-L12-P5, P7-L12-P6, P7-L12-P7, P7-L12-P8, P7-L13-P1, P7-L13-P2, P7-L13-P3, P7-L13-P4, P7-L13-P5, P7-L13-P6, P7-L13-P7, P7-L13-P8, P7-L14-P1, P7-L14-P2, P7-L14-P3, P7-L14-P4, P7-L14-P5, P7-L14-P6, P7-L14-P7, P7-L14-P8, P7-L15-P1, P7-L15-P2, P7-L15-P3, P7-L15-P4, P7-L15-P5, P7-L15-P6, P7-L15-P7, P7-L15-P8, P7-L16-P1, P7-L16-P2, P7-L16-P3, P7-L16-P4, P7-L16-P5, P7-L16-P6, P7-L16-P7, P7-L16-P8, P7-L17-P1, P7-L17-P2, P7-L17-P3, P7-L17-P4, P7-L17-P5, P7-L17-P6, P7-L17-P7, P7-L17-P8, P7-L18-P1, P7-L18-P2, P7-L18-P3, P7-L18-P4, P7-L18-P5, P7-L18-P6, P7-L18-P7, P7-L18-P8, P7-L19-P1, P7-L19-P2, P7-L19-P3, P7-L19-P4, P7-L19-P5, P7-L19-P6, P7-L19-P7, P7-L19-P8, P7-L20-P1, P7-L20-P2, P7-L20-P3, P7-L20-P4, P7-L20-P5, P7-L20-P6, P7-L20-P7, P7-L20-P8, P7-L21-P1, P7-L21-P2, P7-L21-P3, P7-L21-P4, P7-L21-P5, P7-L21-P6, P7-L21-P7, P7-L21-P8, P7-L22-P1, P7-L22-P2, P7-L22-P3, P7-L22-P4, P7-L22-P5, P7-L22-P6, P7-L22-P7, P7-L22-P8, P7-L23-P1, P7-L23-P2, P7-L23-P3, P7-L23-P4, P7-L23-P5, P7-L23-P6, P7-L23-P7, P7-L23-P8, P7-L24-P1, P7-L24-P2, P7-L24-P3, P7-L24-P4, P7-L24-P5, P7-L24-P6, P7-L24-P7, P7-L24-P8, P7-L25-P1, P7-L25-P2, P7-L25-P3, P7-L25-P4, P7-L25-P5, P7-L25-P6, P7-L25-P7, P7-L25-P8, P7-L26-P1, P7-L26-P2, P7-L26-P3, P7-L26-P4, P7-L26-P5, P7-L26-P6, P7-L26-P7, P7-L26-P8, P7-L27-P1, P7-L27-P2, P7-L27-P3, P7-L27-P4, P7-L27-P5, P7-L27-P6, P7-L27-P7, P7-L27-P8, P7-L28-P1, P7-L28-P2, P7-L28-P3, P7-L28-P4, P7-L28-P5, P7-L28-P6, P7-L28-P7, P7-L28-P8, P7-L29-P1, P7-L29-P2, P7-L29-P3, P7-L29-P4, P7-L29-P5, P7-L29-P6, P7-L29-P7, P7-L29-P8, P7-L30-P1, P7-L30-P2, P7-L30-P3, P7-L30-P4, P7-L30-P5, P7-L30-P6, P7-L30-P7, P7-L30-P8, P7-L31-P1, P7-L31-P2, P7-L31-P3, P7-L31-P4, P7-L31-P5, P7-L31-P6, P7-L31-P7, P7-L31-P8, P7-L32-P1, P7-L32-P2, P7-L32-P3, P7-L32-P4, P7-L32-P5, P7-L32-P6, P7-L32-P7, P7-L32-P8, P7-L33-P1, P7-L33-P2, P7-L33-P3, P7-L33-P4, P7-L33-P5, P7-L33-P6, P7-L33-P7, P7-L33-P8, P7-L34-P1, P7-L34-P2, P7-L34-P3, P7-L34-P4, P7-L34-P5, P7-L34-P6, P7-L34-P7, P7-L34-P8, P7-L35-P1, P7-L35-P2, P7-L35-P3, P7-L35-P4, P7-L35-P5, P7-L35-P6, P7-L35-P7, P7-L35-P8, P7-L36-P1, P7-L36-P2, P7-L36-P3, P7-L36-P4, P7-L36-P5, P7-L36-P6, P7-L36-P7, P7-L36-P8, P8-L1-P1, P8-L1-P2, P8-L1-P3, P8-L1-P4, P8-L1-P5, P8-L1-P6, P8-L1-P7, P8-L1-P8, P8-L2-P1, P8-L2-P2, P8-L2-P3, P8-L2-P4, P8-L2-P5, P8-L2-P6, P8-L2-P7, P8-L2-P8, P8-L3-P1, P8-L3-P2, P8-L3-P3, P8-L3-P4, P8-L3-P5, P8-L3-P6, P8-L3-P7, P8-L3-P8, P8-L4-P1, P8-L4-P2, P8-L4-P3, P8-L4-P4, P8-L4-P5, P8-L4-P6, P8-L4-P7, P8-L4-P8, P8-L5-P1, P8-L5-P2, P8-L5-P3, P8-L5-P4, P8-L5-P5, P8-L5-P6, P8-L5-P7, P8-L5-P8, P8-L6-P1, P8-L6-P2, P8-L6-P3, P8-L6-P4, P8-L6-P5, P8-L6-P6, P8-L6-P7, P8-L6-P8, P8-L7-P1, P8-L7-P2, P8-L7-P3, P8-L7-P4, P8-L7-P5, P8-L7-P6, P8-L7-P7, P8-L7-P8, P8-L8-P1, P8-L8-P2, P8-L8-P3, P8-L8-P4, P8-L8-P5, P8-L8-P6, P8-L8-P7, P8-L8-P8, P8-L9-P1, P8-L9-P2, P8-L9-P3, P8-L9-P4, P8-L9-P5, P8-L9-P6, P8-L9-P7, P8-L9-P8, P8-L10-P1, P8-10-P2, P8-10-P3, P8-L10-P4, P8-10-P5, P8-10-P6, P8-10-P7, P8-10-P8, P8-L11-P1, P8-L11-P2, P8-L11-P3, P8-L11-P4, P8-L11-P5, P8-L11-P6, P8-L11-P7, P8-L11-P8, P8-L12-P1, P8-L12-P2, P8-L12-P3, P8-L12-P4, P8-L12-P5, P8-L12-P6, P8-L12-P7, P8-L12-P8, P8-L13-P1, P8-L13-P2, P8-L13-P3, P8-L13-P4, P8-L13-P5, P8-L13-P6, P8-L13-P7, P8-L13-P8, P8-L14-P1, P8-L14-P2, P8-L14-P3, P8-L14-P4, P8-L14-P5, P8-L14-P6, P8-L14-P7, P8-L14-P8, P8-L15-P1, P8-L15-P2, P8-L15-P3, P8-L15-P4, P8-L15-P5, P8-L15-P6, P8-L15-P7, P8-L15-P8, P8-L16-P1, P8-L16-P2, P8-L16-P3, P8-L16-P4, P8-L16-P5, P8-L16-P6, P8-L16-P7, P8-L16-P8, P8-L17-P1, P8-L17-P2, P8-L17-P3, P8-L17-P4, P8-L17-P5, P8-L17-P6, P8-L17-P7, P8-L17-P8, P8-L18-P1, P8-L18-P2, P8-L18-P3, P8-L18-P4, P8-L18-P5, P8-L18-P6, P8-L18-P7, P8-L18-P8, P8-L19-P1, P8-L19-P2, P8-L19-P3, P8-L19-P4, P8-L19-P5, P8-L19-P6, P8-L19-P7, P8-L19-P8, P8-L20-P1, P8-L20-P2, P8-L20-P3, P8-L20-P4, P8-L20-P5, P8-L20-P6, P8-L20-P7, P8-L20-P8, P8-L21-P1, P8-L21-P2, P8-L21-P3, P8-L21-P4, P8-L21-P5, P8-L21-P6, P8-L21-P7, P8-L21-P8, P8-L22-P1, P8-L22-P2, P8-L22-P3, P8-L22-P4, P8-L22-P5, P8-L22-P6, P8-L22-P7, P8-L22-P8, P8-L23-P1, P8-L23-P2, P8-L23-P3, P8-L23-P4, P8-L23-P5, P8-L23-P6, P8-L23-P7, P8-L23-P8, P8-L24-P1, P8-L24-P2, P8-L24-P3, P8-L24-P4, P8-L24-P5, P8-L24-P6, P8-L24-P7, P8-L24-P8, P8-L25-P1, P8-L25-P2, P8-L25-P3, P8-L25-P4, P8-L25-P5, P8-L25-P6, P8-L25-P7, P8-L25-P8, P8-L26-P1, P8-L26-P2, P8-L26-P3, P8-L26-P4, P8-L26-P5, P8-L26-P6, P8-L26-P7, P8-L26-P8, P8-L27-P1, P8-L27-P2, P8-L27-P3, P8-L27-P4, P8-L27-P5, P8-L27-P6, P8-L27-P7, P8-L27-P8, P8-L28-P1, P8-L28-P2, P8-L28-P3, P8-L28-P4, P8-L28-P5, P8-L28-P6, P8-L28-P7, P8-L28-P8, P8-L29-P1, P8-L29-P2, P8-L29-P3, P8-L29-P4, P8-L29-P5, P8-L29-P6, P8-L29-P7, P8-L29-P8, P8-L30-P1, P8-L30-P2, P8-L30-P3, P8-L30-P4, P8-L30-P5, P8-L30-P6, P8-L30-P7, P8-L30-P8, P8-L31-P1, P8-L31-P2, P8-L31-P3, P8-L31-P4, P8-L31-P5, P8-L31-P6, P8-L31-P7, P8-L31-P8, P8-L32-P1, P8-L32-P2, P8-L32-P3, P8-L32-P4, P8-L32-P5, P8-L32-P6, P8-L32-P7, P8-L32-P8, P8-L33-P1, P8-L33-P2, P8-L33-P3, P8-L33-P4, P8-L33-P5, P8-L33-P6, P8-L33-P7, P8-L33-P8, P8-L34-P1, P8-L34-P2, P8-L34-P3, P8-L34-P4, P8-L34-P5, P8-L34-P6, P8-L34-P7, P8-L34-P8, P8-L35-P1, P8-L35-P2, P8-L35-P3, P8-L35-P4, P8-L35-P5, P8-L35-P6, P8-L35-P7, P8-L35-P8, P8-L36-P1, P8-L36-P2, P8-L36-P3, P8-L36-P4, P8-L36-P5, P8-L36-P6, P8-L36-P7, P8-L36-P8,

According to an aspect of the invention, there is provided a conjugate comprising an antibody or an antigen-binding fragment thereof covalently linked to two pharmaceutically active drugs through a dual linker, and wherein the dual linker has one attachment point connected to the antibody and two attachment points to the two pharmaceutically active drugs, with the proviso that neither pharmaceutically active drug is a BH3 mimetic. In one embodiment, the two pharmaceutically active drugs are the same or different. In another embodiment, the pharmaceutically active drug is selected from a cytotoxic drug, a cytostatic drug and an immunosuppressive drug. In yet another embodiment, the pharmaceutically active drug is selected from an auristatin, a camptothecin, a duocarmycin, an etoposide, a maytansine, a maytansinoid, a taxane, a benzodiazepine or benzodiazepine containing drug (e.g., pyrrolo[1,4]-benzodiazepines (PBDs), indolinobenzodiazepines, and oxazolidinobenzodiazepines), and a vinca alkaloid. In an embodiment, the pharmaceutically active drug is an auristatin. In an embodiment, the pharmaceutically active drug is selected from an antitubulin agent, a tubulin inhibitor, a DNA minor groove binder, a DNA replication inhibitor, an alkylating agent, an antibiotic, an antifolate, an antimetabolite, a chemotherapy sensitizer, a topoisomerase inhibitor, and/or a vinca alkaloid. In another embodiment, the pharmaceutically active drug is selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor, a DHFR inhibitor, or a pro-apoptotic agent. In a further embodiment, the pharmaceutically active drug is selected from a topoisomerase 1 inhibitor, or an enantiomer, diastereoisomer, atropisomer, deuterated derivative, and/or addition salt thereof with a pharmaceutically acceptable acid or base. In an embodiment, the pharmaceutically active drug is wherein the topoisomerase 1 inhibitor is selected from topotecan, exatecan, deruxtecan and SN-38. In yet another embodiment, the pharmaceutically active drug is selected from an anti-mitotic drug, or an enantiomer, diastereoisomer, atropisomer, deuterated derivative, and/or addition salt thereof with a pharmaceutically acceptable acid or base. In a further embodiment, the anti-mitotic drug is monomethyl auristatin E (MMAE) or a taxane. In an embodiment, the taxane is selected from docetaxel, paclitaxel, or cabazitaxel. In another embodiment, the conjugate comprises any of the compounds or pharmaceutically acceptable salts thereof disclosed herein.

As mentioned above, according to an aspect of the invention, there is provided a conjugate of formula (A-2):

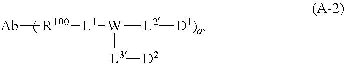

wherein:
Ab is an antibody or fragment thereof;
R¹⁰⁰ is a coupling group;
L¹ is a bridging spacer;
W is a branching moiety;
L^{2′} and L^{3′} are each independently a linker;
D¹ and D² are each independently a pharmaceutically active drug, optionally with the proviso that
neither D¹ nor D² is a BH3 mimetic; and
a is an integer from 1 to 16.

In an Embodiment, each of L^{2′} and L^{3′} comprises a cleavable group, optionally at least one cleavable group comprises a phosphate group, a pyrophosphate group and/or a self-immolative group (e.g. a peptide group and a self-immolative group).

In an Embodiment, each of L^{2′} and L^{3′} comprises a peptide group and a self-immolative group.

In an Embodiment, each of L^{2′} and L^{3′} comprises a cleavable group, optionally at least one cleavable group comprises a phosphate group, a pyrophosphate group, a sugar (e.g. glucuronamide) group, a peptide group, and/or a self-immolative group (e.g. a peptide group and a self-immolative group).

In an Embodiment, the conjugate is of formula (B-2):

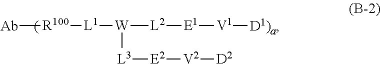

wherein:
Ab is an antibody or fragment thereof;
R¹⁰⁰ is a coupling group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, an enzyme cleavage element or a hydrophilic moiety (e.g. E¹ and E² are each an enzyme cleavage element);
V¹ and V² independently comprise i) a self-immolative group, ii) an enzyme cleavage element (e.g. V¹ and V² are each independently a self-immolative group) or iii) a self-immolative group and an enzyme cleavage element;
D¹ and D² are each independently a pharmaceutically active drug, with the proviso that neither D¹ nor D² is a BH3 mimetic; and
A is an integer from 1 to 16.

In an Embodiment, the conjugate is of formula (B-2):

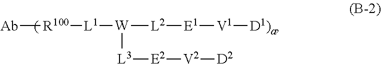

wherein:
Ab is an antibody or fragment thereof;
R¹⁰⁰ is a coupling group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, a peptide group comprising 1 to 6 amino acids, wherein said peptide group is optionally substituted by a hydrophilic moiety;
V¹ and V² are each independently i) a self-immolative group, or ii) an enzyme cleavage element (e.g. V¹ and V² are each independently a self-immolative group);
D¹ and D² are each independently a pharmaceutically active drug, optionally with the proviso that neither D¹ nor D² is a BH3 mimetic; and
a is an integer from 1 to 16.

In an Embodiment, W is N or CH.

In an Embodiment, E¹ and E² are each an enzyme cleavage element.

In an Embodiment, V¹ and V² are each independently a self-immolative group

In an Embodiment, a is an integer from 1 to 8, or from 1 to 6 or from 1 to 4 or a is 1 or 2.

In an Embodiment, a is determined by liquid chromatography-mass spectrometry (LC-MS).

In an Embodiment, (i) the cleavable linker comprises a phosphate group, a pyrophosphate group and/or a self-immolative group; (ii) the cleavable linker comprises a self-immolative group; or (iii) the cleavable linker comprises a self-immolative group comprising —CH₂—O—, —NH—CH₂—, —C(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.

In an Embodiment, W is N or CH.

In an Embodiment, R² and R³ are each a hydrophilic group.

In an Embodiment, the conjugate is of formula (C-2):

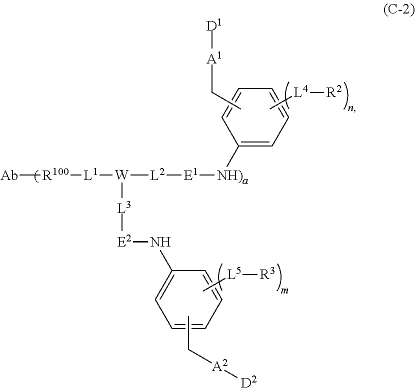

wherein:
Ab is an antibody or antigen-binding fragment thereof;
R¹⁰⁰ is a coupling group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently a peptide group comprising 1 to 6 amino acids, wherein said peptide group is optionally substituted by a hydrophilic group;
A¹ and A² are each independently a bond, —OC(═O)—*,

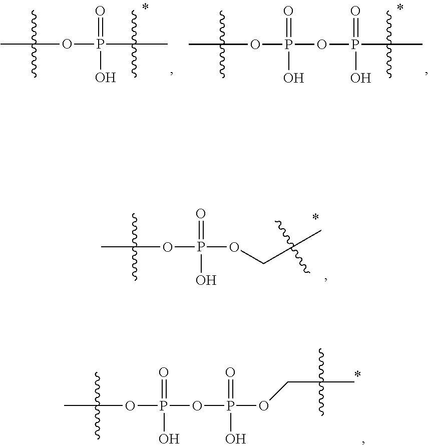 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆alkyl, and C₃-C₈cycloalkyl and the * of
A¹ or A² indicates the point of attachment to D¹ or D²;
D¹ and D² are each independently a pharmaceutically active drug, optionally with the proviso that neither D¹ nor D² is a BH3 mimetic;
L⁴ and L⁵ are each independently a spacer moiety;
R² and R³ are each independently a hydrophilic group or an enzyme cleavage element (e.g. R² and R³ are each a hydrophilic group); and
m and n are each independently 0 or 1.

In an Embodiment, the conjugate is of formula (D1-2), (D²-2) or (D³-2):

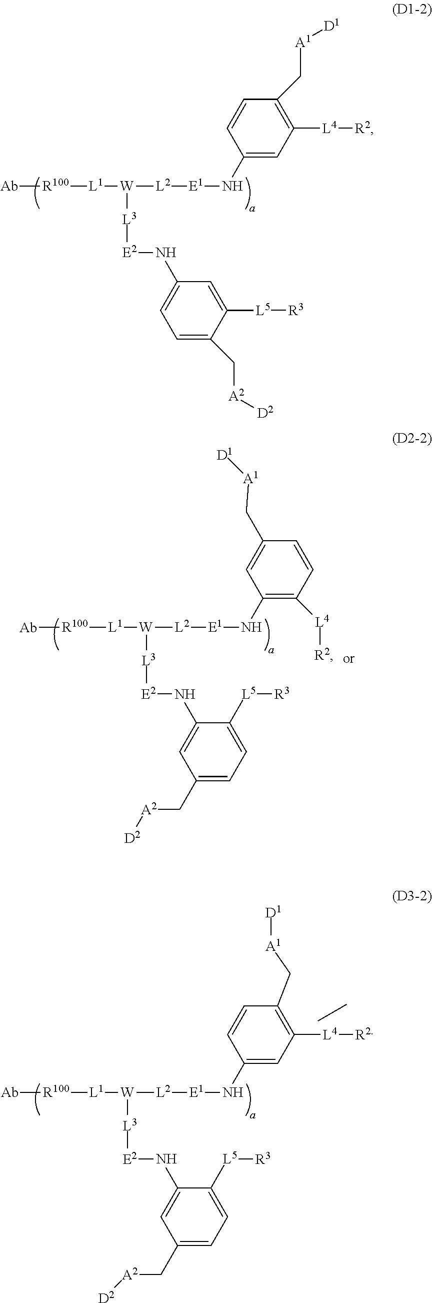

In an Embodiment, the conjugate is of Formula (D1-2), R² and R³ are each independently a hydrophilic group, wherein for Formula (D2-2), R² and R³ are each independently an enzyme cleavage element; and for Formula (D3-2), R² is a hydrophilic group and R³ is an enzyme cleavage element.

In an Embodiment, R¹⁰⁰ is selected from the group consisting of

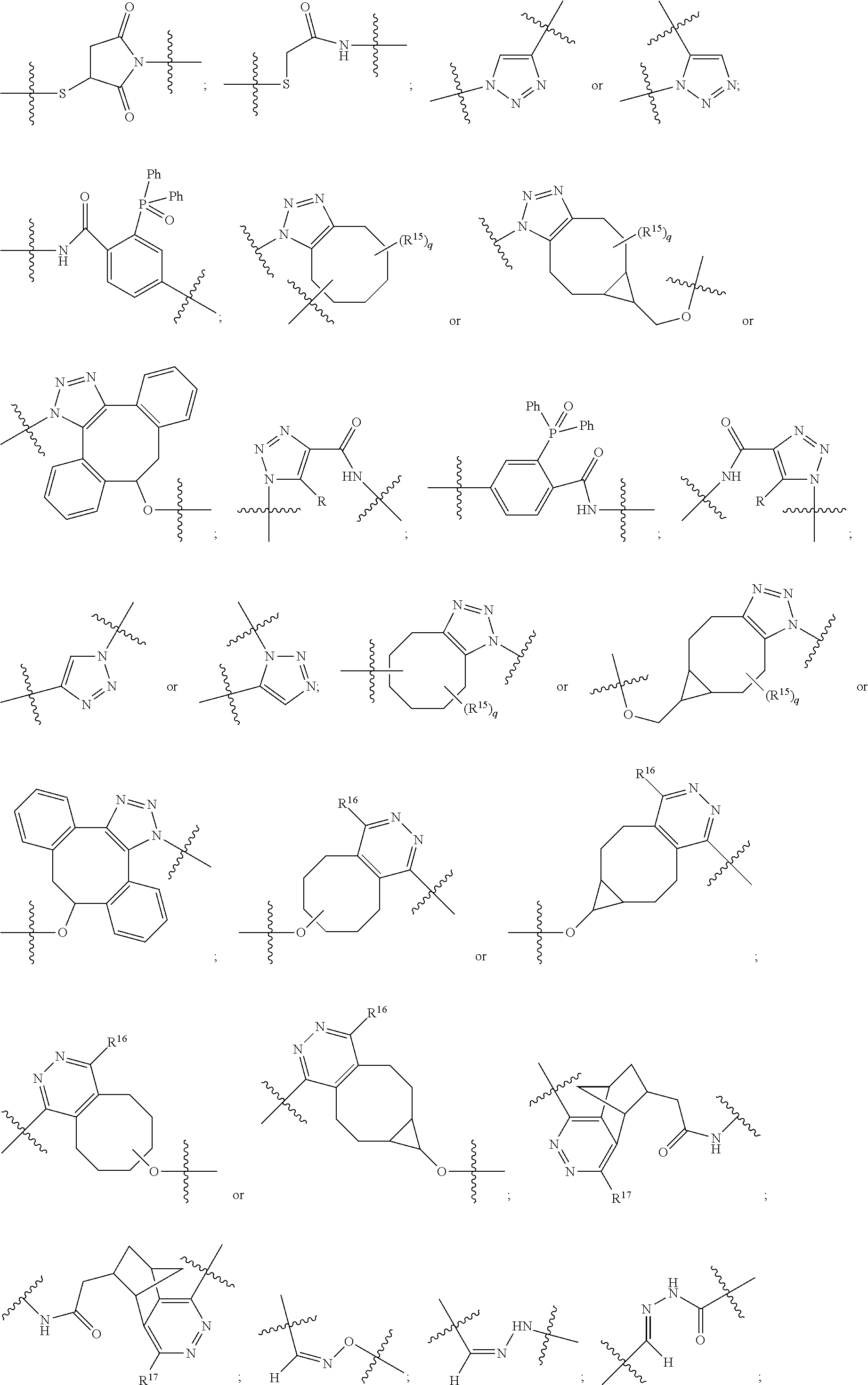

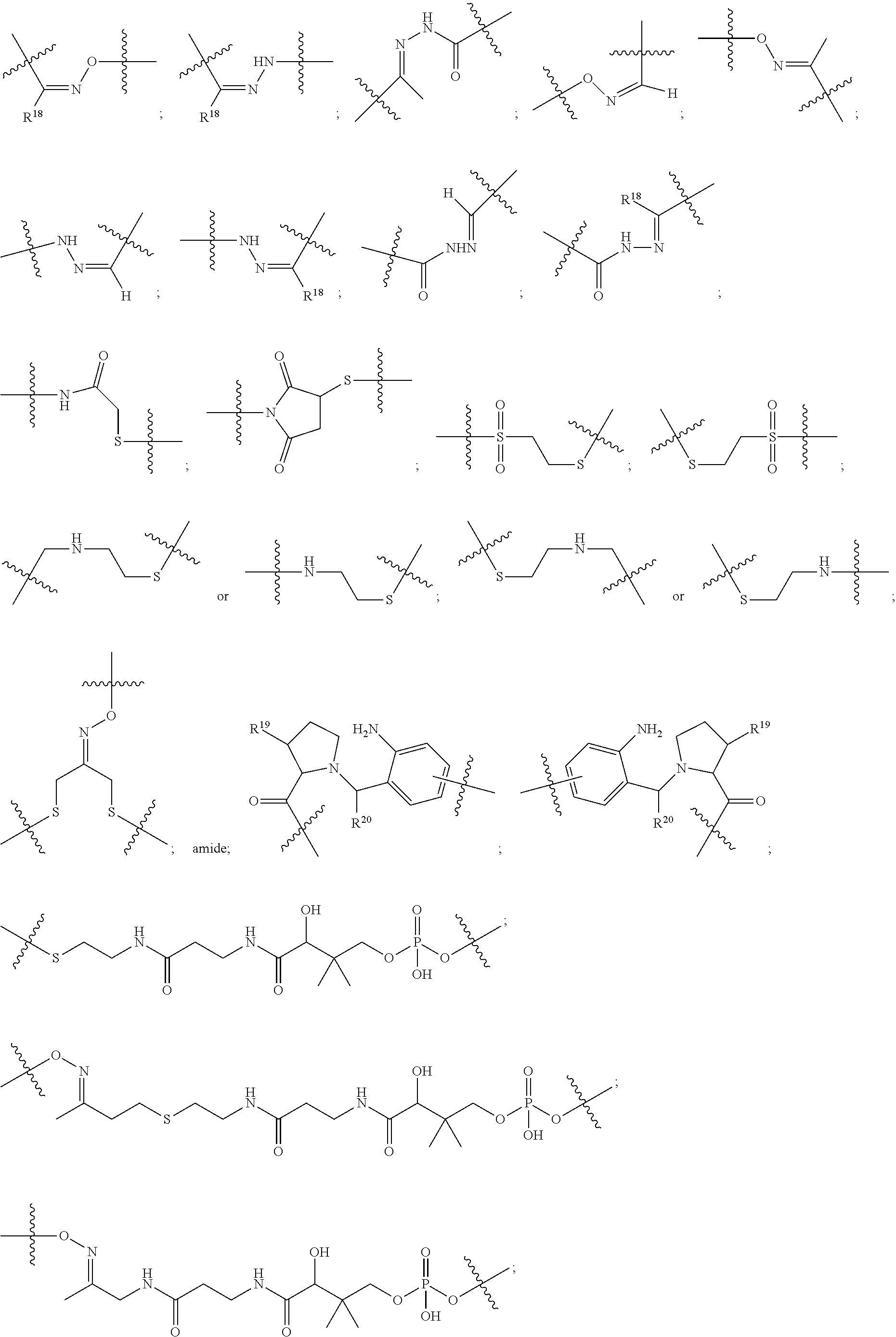

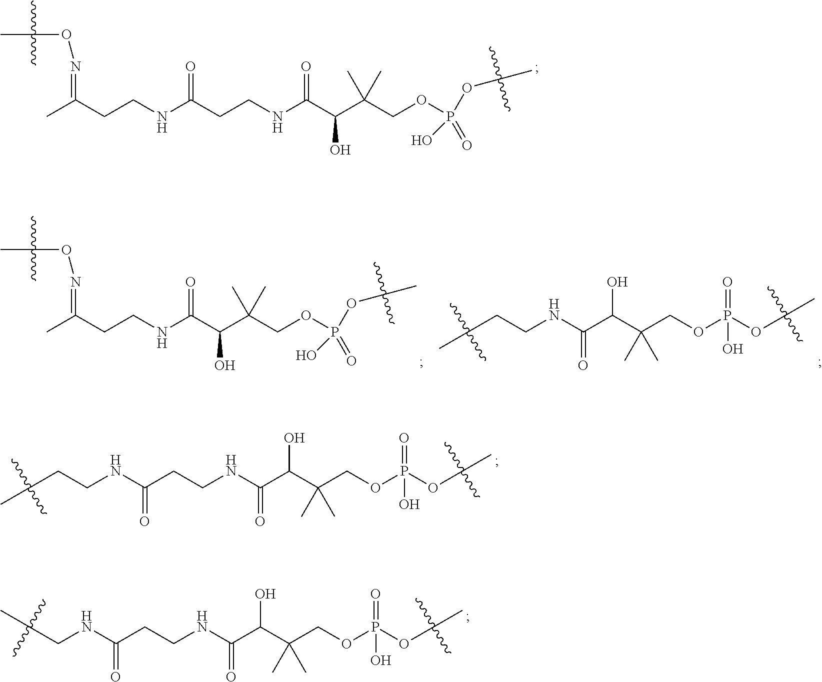 [SMILES: *O/N=C(\C)CCNC(=O)CCNC(=O)[C@H](O)C(C)(C)COP(=O)(O)O* ; *O/N=C(\C)CCNC(=O)[C@H](O)C(C)(C)COP(=O)(O)O* ; *CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O*]

and
disulfide,
   wherein:
R¹⁶ is H, C₁-C₄alkyl, phenyl, pyrimidine or pyridine;
R¹⁸ is H, C₁-C₆alkyl, phenyl or C₁-C₄alkyl substituted with 1 to 3 —OH groups;
each R¹⁵ is independently selected from H, C₁-C₆alkyl, fluoro, benzyloxy substituted with —C(═O)OH, benzyl substituted with —C(═O)OH, C₁-C₄alkoxy substituted with —C(═O)OH and C₁-C₄alkyl substituted with —C(═O)OH;
R¹⁷ is independently selected from H, phenyl and pyridine;
q is 0, 1, 2 or 3;
R¹⁹ is H or methyl; and
R²⁰ is H, —CH₃ or phenyl.

In an Embodiment, R¹⁰⁰ is selected from the group consisting of

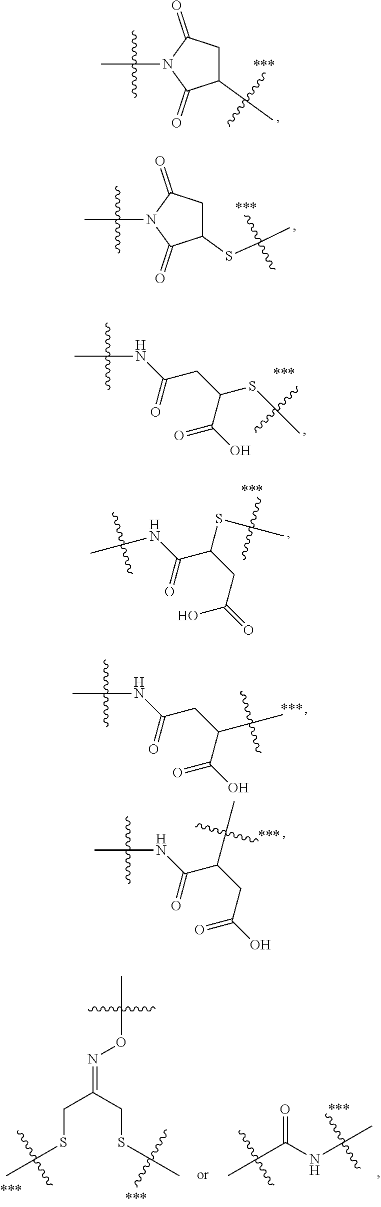

where the *** of R¹⁰⁰ indicates the point of attachment to Ab.

In an Embodiment, R¹⁰⁰ is

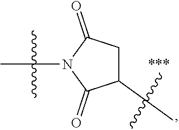 [SMILES: *C1CC(=O)N(*)C1=O]

where the *** of R¹⁰⁰ indicates the point of attachment to Ab.

In an Embodiment, (1) L¹ comprises:

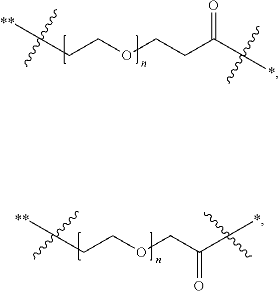

### *—CH(OH)CH(OH)CH(OH)CH(OH)—**,

wherein each n is an integer from 1 to 12, wherein the * of L¹ indicates the point of direct or indirect (e.g. direct) attachment to W, and the ** of L¹ indicates the point of direct or indirect (e.g. direct) attachment to R¹;
(2) L¹ is

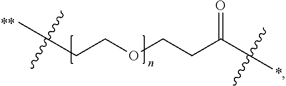

and n is an integer from 1 to 12 or n is 1 or n is 12, wherein the * of L¹ indicates the point of direct or indirect (e.g. direct) attachment to W, and the ** of L¹ indicates the point of direct or indirect (e.g. direct) attachment to R¹;
(3) L¹ is

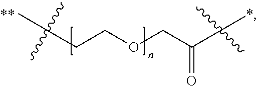

and n is an integer from 1 to 12, wherein the * of L¹ indicates the point of direct or indirect (e.g. direct) attachment to W, and the ** of L₁ indicates the point of direct or indirect (e.g. direct) attachment to R¹;
(4) L¹ comprises

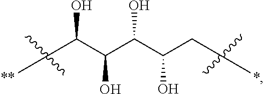 [SMILES: *C[C@H](O)[C@@H](O)[C@@H](O)[C@@H](*)O]

wherein the * of L¹ indicates the point of direct or indirect (e.g. direct) attachment to W, and the ** of L¹ indicates the point of direct or indirect (e.g. direct) attachment to R¹; or
(5) L¹ is a bridging spacer comprising: *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R^{L1})₂(CH₂)ₘC(═O)NR^{L1} (CH₂)ₘNR^{L1}C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R^{L1})₂—** or *—C(═O)(CH₂)ₘC(═O)NH(CH₂)ₘ—**, wherein the * of L¹ indicates the point of direct or indirect (e.g. direct) attachment to W, and the ** of L¹ indicates the point of direct or indirect (e.g. direct) attachment to R¹;
X₁ is

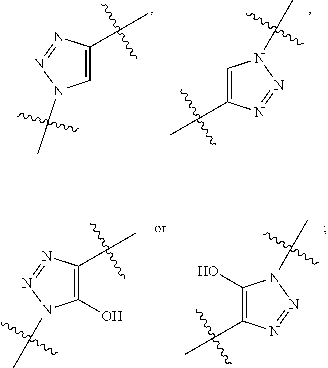 [SMILES: *c1cn(*)nn1 ; *c1cn(*)nn1 ; *c1nnn(*)c1O ; *c1nnn(*)c1O]

and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30; and
each R^{L1} is independently selected from H and C₁-C₆alkyl.

In an Embodiment, L¹ comprises a moiety represented by

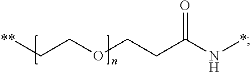

wherein n is an integer from 1 to 12 (e.g. 4 to 8, e.g. 4 or 8), wherein the * of L¹ indicates the point of direct or indirect (e.g. direct) attachment to W, and the ** of L¹ indicates the point of direct or indirect attachment to R¹.

In an Embodiment, n is an integer from 4 to 8.

In an Embodiment, n is 4 or 8.

In an Embodiment, L¹ is represented by a formula

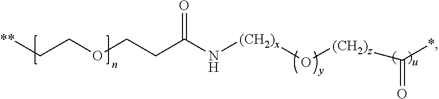

wherein
n is an integer from 1 to 12 (e.g. 4 to 8, e.g. 4 or 8);
x is an integer from 0 to 6 (e.g. 0 to 4, e.g. 0 to 2, e.g. 0 or 2);
y is 0 or 1;
z is an integer from 0 to 6 (e.g. 0 to 4, e.g. 0 to 2, e.g. 0 or 2);
u is 0 or 1; and
wherein the * of L¹ indicates the point of direct attachment to W, and the ** of L¹ indicates the point of direct attachment to R¹.

In an Embodiment, n is an integer from 4 to 8.

In an Embodiment, n is 4 or 8.

In an Embodiment, x is an integer from 0 to 2.

In an Embodiment, x is 0 or 2.

In an Embodiment, z is an integer from 0 to 2.

In an Embodiment, z is 0 or 2.

In an Embodiment, L¹ is selected from the group consisting of:

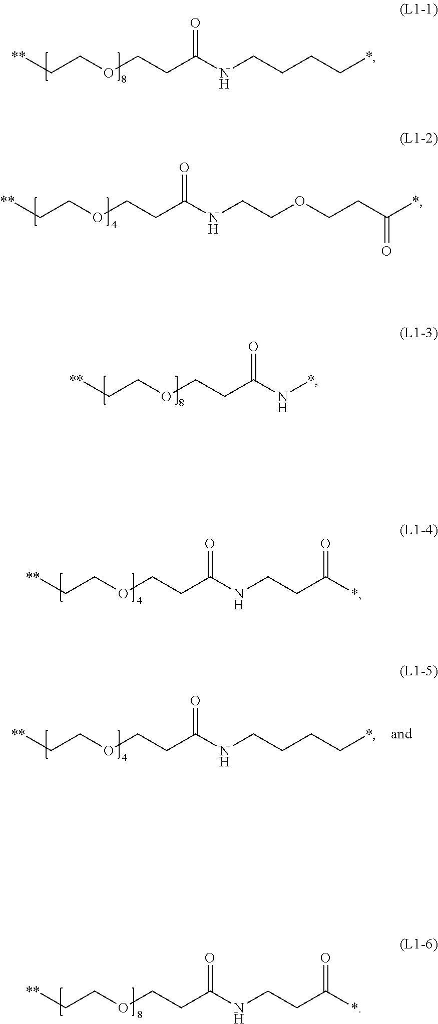

In an Embodiment, L² and L³ are each independently a connecting spacer comprising a moiety represented by:

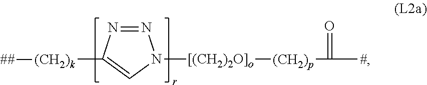

wherein
k is an integer from 0 to 6;
r is 0 or 1;
o is an integer from 0 to 12;
p is an integer from 0 to 6; and
   wherein the # of L2 or L3 indicates the point of direct or indirect attachment to E¹ or E², respectively, and the ## of L2 or L3 indicates the point of direct or indirect attachment to W.

In an Embodiment, L² and L³ are each independently a connecting spacer selected from a group consisting of:

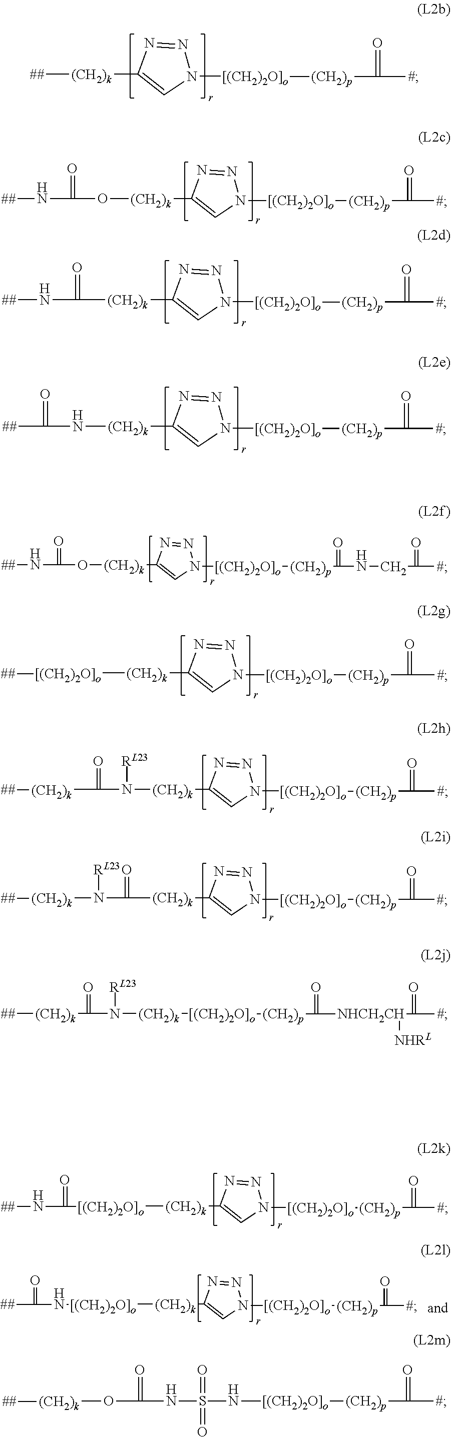

wherein
k, in each occurrence, is independently an integer from 0 to 4;
r, in each occurrence, is independently 0 or 1;
o, in each occurrence, is independently an integer from 0 to 10;
p, in each occurrence, is independently an integer from 0 to 4;
R^{L23} is hydrogen or C₁-C₆alkyl;
R^{L} is hydrogen or —C(O)—R^{H};
R^{H} is a hydrophilic group; and
the # of L² or L³ indicates the point of direct attachment to E¹ or E², respectively, and the
## of L² or L³ indicates the point of direct attachment to W;
provided that when W is N, L² and L³ are not (L2c), (L2d), (L2f) or (L2k).

In an Embodiment, L² and L³ are each independently a connecting spacer selected from a group consisting of:

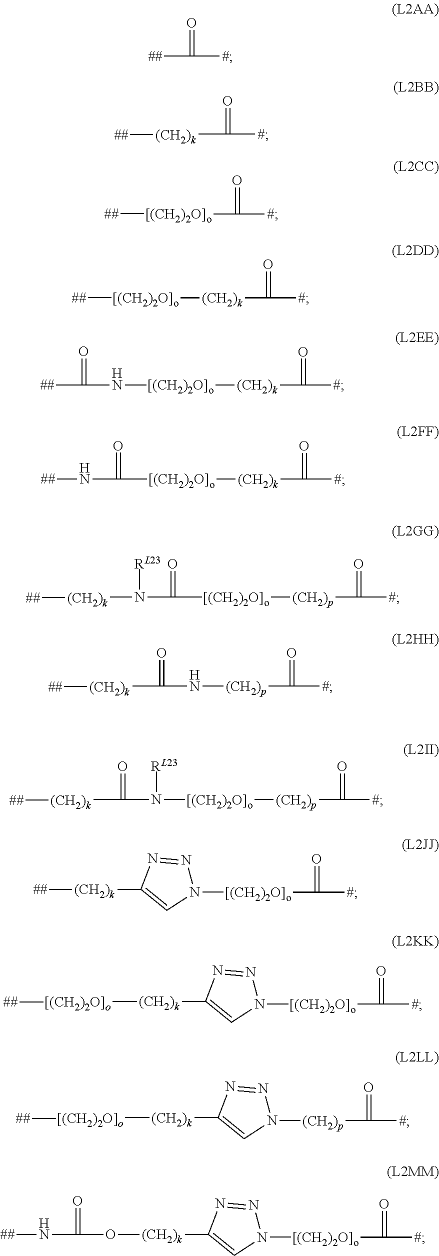

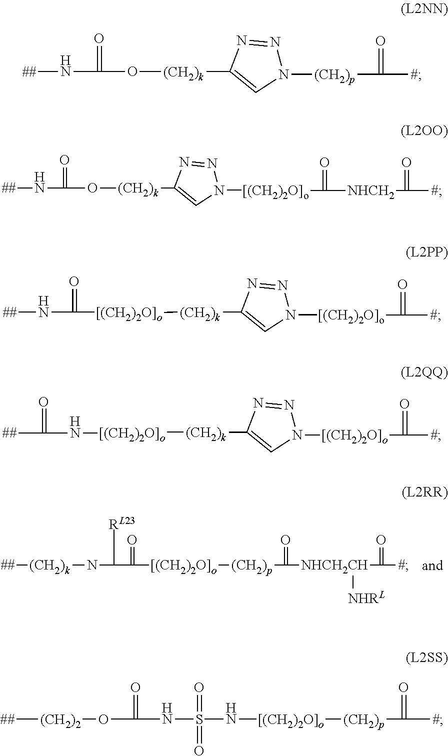

wherein
k, in each occurrence, is independently an integer from 1 to 3;
o, in each occurrence, is independently an integer from 1 to 9;
p, in each occurrence, is independently an integer from 1 to 3;
R^{L23} is hydrogen or C₁-C₃alkyl;
R^{L} is hydrogen or —C(O)—R^{H};
R^{H} is a hydrophilic group; and
the # of L² or L³ indicates the point of direct attachment to E¹ or E², respectively, and the ## of L² or L³ indicates the point of direct attachment to W; provided that when W is N, L² and L³ are not (L2FF), (L2MM), (L2NN), (L2OO), or (L2PP).

In an Embodiment, L² and L³, independently, are a connecting spacer selected from a group consisting of:

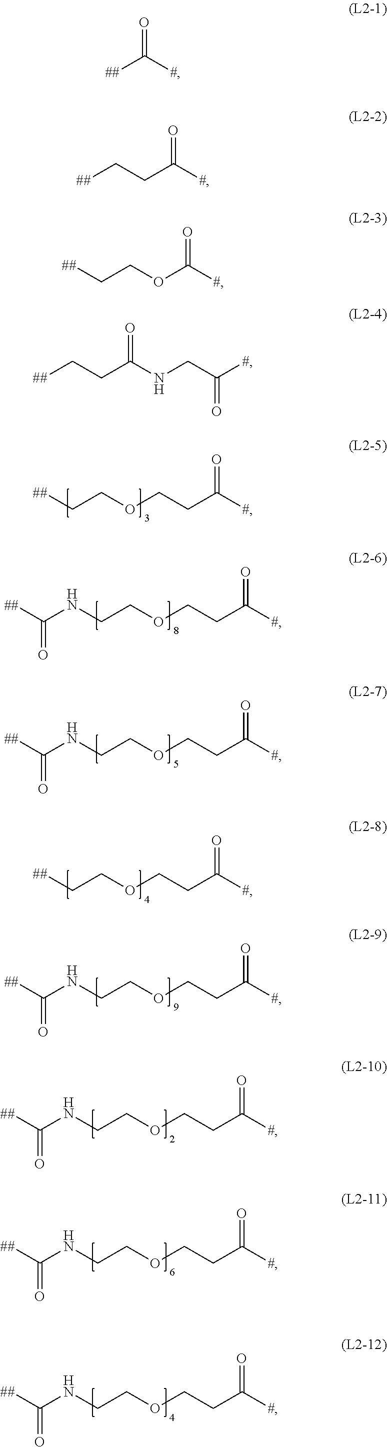

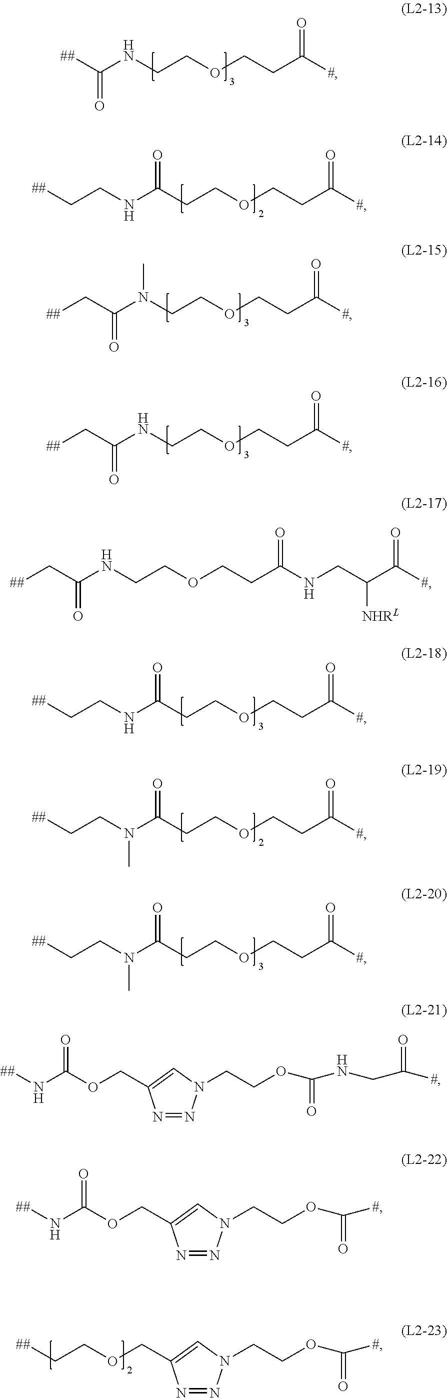

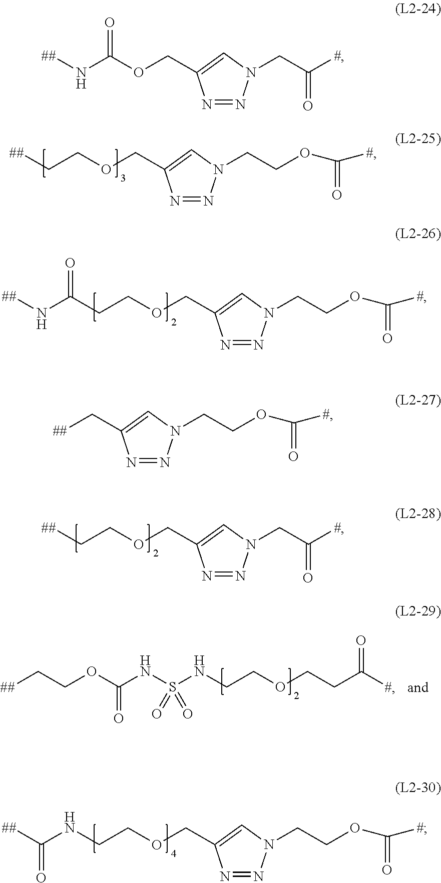

wherein the # of L² or L³ indicates the point of direct attachment to E¹ or E², respectively, the ## of L² or L³ indicates the point of direct attachment to W; R^{L} is hydrogen or —C(O)—R^{H}; and
R^{H} is

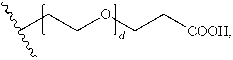

and d is an integer from 20 to 30 (e.g. 25).

In an Embodiment, d is 25.

In an Embodiment, each peptide group independently comprises 1 to 4, 1 to 3, or 1 to 2 amino acid residues.

In an Embodiment, each peptide group independently comprises 2 amino acid residues.

In an Embodiment, each amino acid residues is independently selected from glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), L-tyrosine (Tyr) and β-alanine (β-Ala).

In an Embodiment, each peptide group is independently selected from Val-Cit, Phe-Lys, Val-Ala, Val-Lys, Leu-Cit, Cit-(β-Ala), Gly-Gly-Gly, Gly-Gly-Phe-Gly, and sulfo-Ala-Val-Ala.

In an Embodiment, E¹ and/or E², independently, is/are each a peptide group selected from a group consisting of:

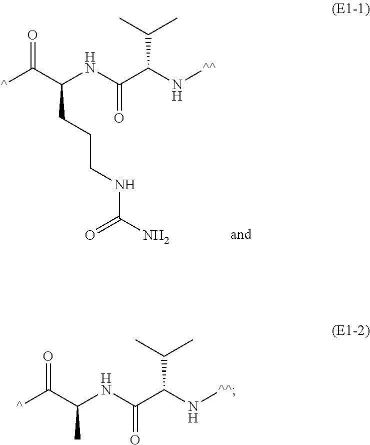

wherein \hat{ } of E1-1 or E1-2 indicates the point of direct attachment to V¹ or V² in Formula (B) or direct attachment to the —NH— group in Formula (C) and (D); and \hat{ }\hat{ } of E1-1 or E1-2 indicates the point of direct attachment to L² or L³, respectively (e.g. where E¹ and E² are each a peptide group independently selected from (E1-1) and (E1-2)).

In an Embodiment, E¹ and E² are each a peptide group independently selected from (E1-1) and (E1-2).

In an Embodiment, E¹ and/or E², independently, is/are each a peptide group represented by

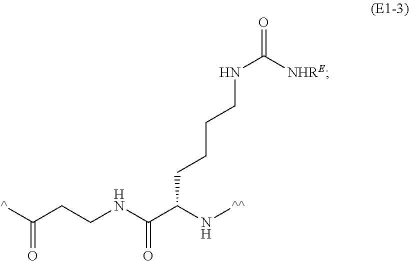

wherein R^{E} is a hydrophilic group R^{H}.

In an Embodiment, the hydrophilic group R^{H} in (E1-3) is

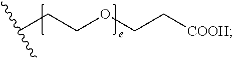

wherein e is an integer between 20 and 30 (e.g. 25).

In an Embodiment, e is 25.

In an Embodiment, E¹ and E² are each

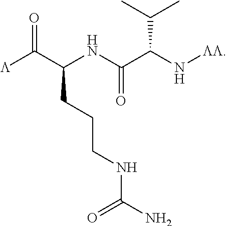

In an Embodiment, A¹ and A² are independently selected from a bond and —OC(═O)—*, wherein * indicates the point of attachment to D¹ or D² respectively.

In an Embodiment, A¹ and A² are independently selected from a bond,

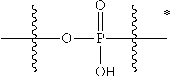 [SMILES: *OP(*)(=O)O]

and —OC(═O)—*, wherein * indicates the point of attachment to D¹ or D² respectively.

In an Embodiment, A¹ and A² are a bond.

In an Embodiment, A¹ and A² are independently a bond or

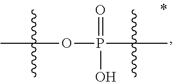 [SMILES: *OP(*)(=O)O]

wherein * indicates the point of attachment to D¹ or D² respectively.

In an Embodiment, one of A¹ and A² is

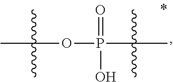 [SMILES: *OP(*)(=O)O]

and the other of A¹ and A² is a bond.

In an Embodiment, A¹ and A² are both

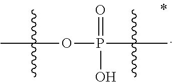 [SMILES: *OP(*)(=O)O]

In an Embodiment, one of A¹ and A² is

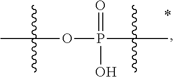 [SMILES: *OP(*)(=O)O]

and the other of A¹ and A² is OC(═O)—*.

In an Embodiment, (i) A¹ and A² are —OC(═O)—*;
(ii) A¹ and A² are

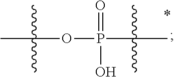 [SMILES: *OP(*)(=O)O]

(iii) A¹ is —OC(═O)—* and A² is a bond;
(iv) A¹ is —OC(═O)—* and A² is

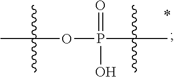 [SMILES: *OP(*)(=O)O]

(v) A¹ is a bond and A² is

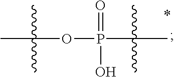 [SMILES: *OP(*)(=O)O]

or
(vi) A¹ is a bond and A² is —OC(═O)—*,
wherein * indicates the point of attachment to D¹ or D².

In an Embodiment, i) L⁴ and L⁵ are each independently a spacer moiety having the structure

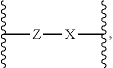

wherein:
Z is —O—, —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{L45}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is a bond, triazolyl, or —CH₂—triazolyl-,
wherein X is connected to R² or R³; or
(ii) L⁴ and L⁵, independently, are a spacer moiety having the structure

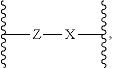

wherein:
Z is —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{b}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is —CH₂—triazolyl-C₁-C₄alkylene-OC(O)NHS(O)₂NH—, —C₄-C₆cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂—triazolyl-C₁-C₄alkylene-OC(O)NHS(O)₂NH—(CH₂CH₂O)ₙ—, —C₄-C₆cycloalkylene-OC(O)NHS(O)₂NH—(CH₂CH₂O)ₙ—, wherein each n independently is 1, 2, or 3,
wherein X is connected to R² or R³.

In an Embodiment, Z is —O—, —CH₂NR^{L45}C(═O)—, —CH₂NR^{L45}C(═O)NH— or —CH₂O—; X is a bond, triazolyl, or —CH₂—triazolyl-; and R^{L45}, in each occurrence, is independently H or C₁-C₃alkyl.

In an Embodiment, L⁴ and L⁵ are each independently a spacer moiety selected from a group consisting of

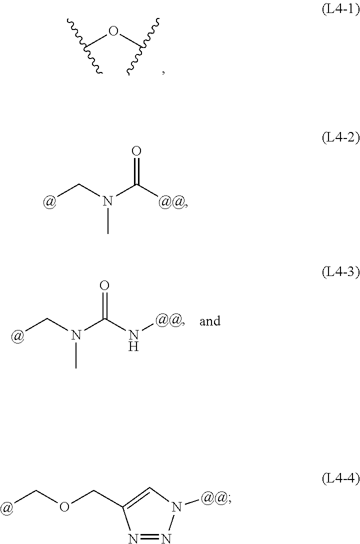

wherein the @ of L⁴ or L⁵ indicates the point of direct attachment to the phenyl group, and the @@ of L⁴ or L⁵ indicates the point of direct attachment to R² or R³.

In an Embodiment, the hydrophilic groups represented by R² and R³ each independently comprises polyethylene glycol, polyalkylene glycol, a polyol, a polysarcosine, a sugar, an oligosaccharide, a polypeptide, C₂-C₆alkyl substituted with 1 to 3

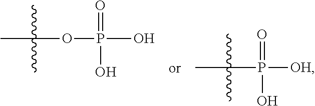 [SMILES: *OP(=O)(O)O ; *P(=O)(O)O]

or C₂—C₆alkyl substituted with 1 to 2 substituents independently selected from —OC(═O)NHS(O)₂NHCH₂CH₂OCH₃, —NHC(═O)C₁-C₄alkylene-P(O)(OCH₂CH₃)₂ and —COOH groups.

In an Embodiment, R² or/and R³ independently is/are each selected from the group consisting of:

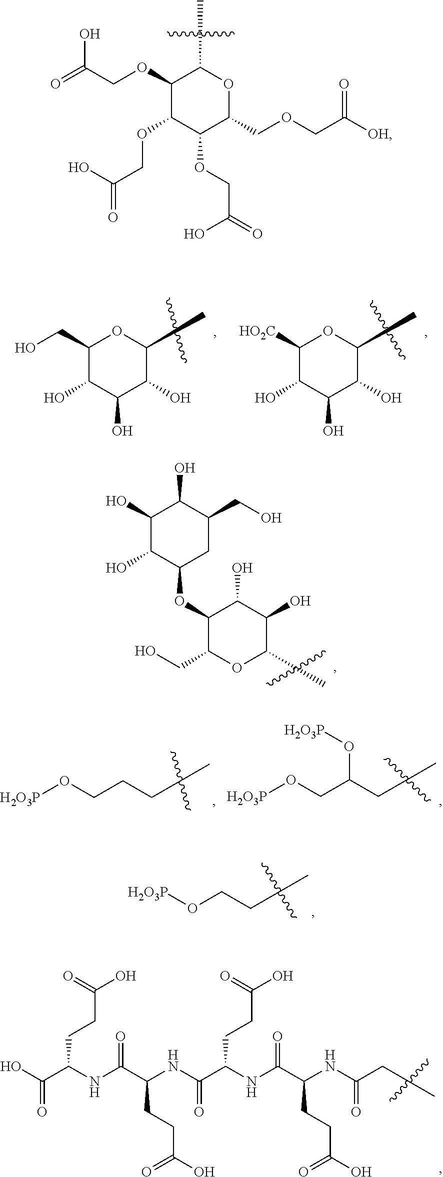

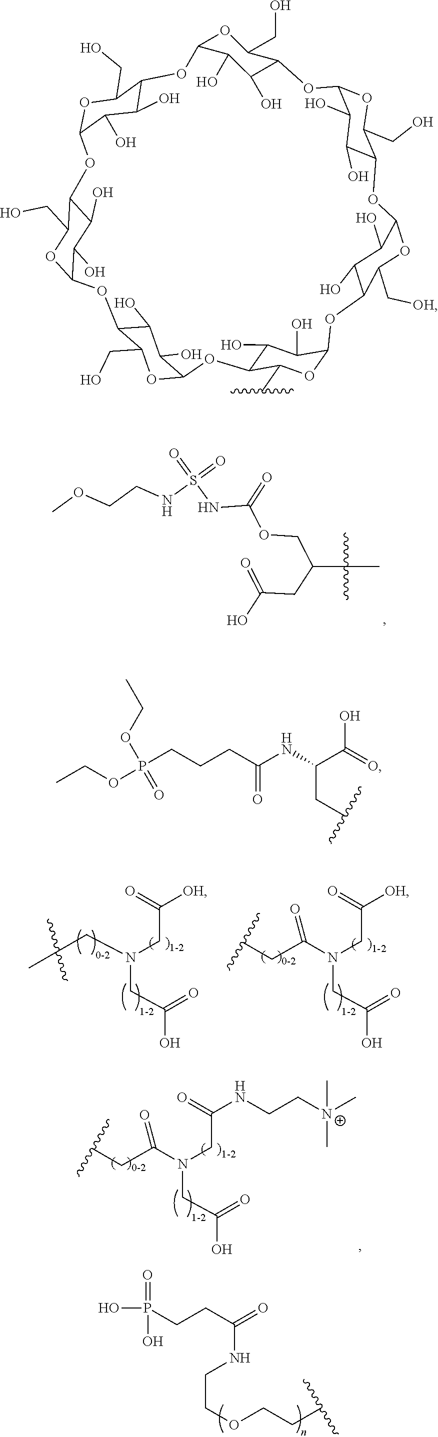

wherein n is an integer between 1 and 6,

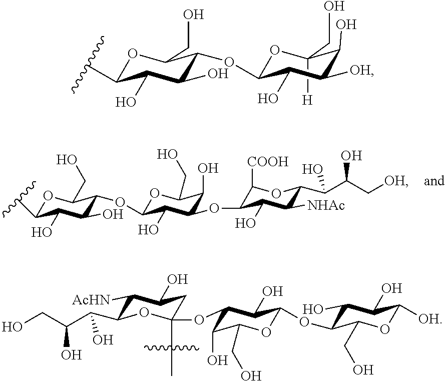

In an Embodiment, R² or/and R³ is/are each independently comprising:
(i) a polysarcosine with the following moiety:

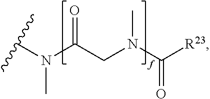

wherein
   f is an integer between 3 and 25; and
   R²³ is H, —CH₃ or —CH₂CH₂C(═O)OH;
(ii) a polyethylene glycol of formula:

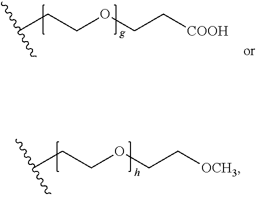

wherein
   g and h are independently an integer between 2 and 30; or
(iii) a moiety represented by

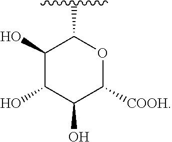 [SMILES: *[C@@H]1O[C@H](C(=O)O)[C@@H](O)[C@H](O)[C@H]1O]

In an embodiment, the hydrophilic group represented by R² or R³ each independently comprises:
(i) a polysarcosine with the following moiety:

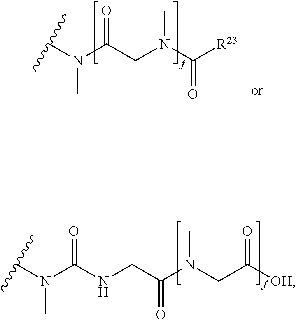

wherein f is an integer between 3 and 25; and R²³ is H, —CH₃ or —CH₂CH₂C(═O)OH; or (ii) a polyethylene glycol of formula:

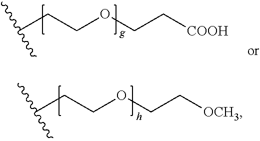

wherein g and h are independently an integer between 2 and 30.

In an Embodiment, R² and R³, independently, is/are each selected from a group consisting of

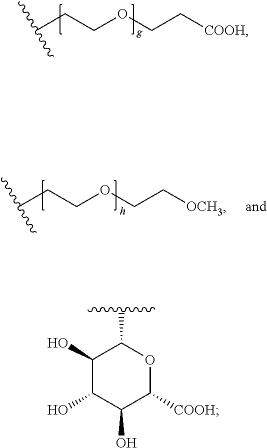

wherein
g and h are independently an integer between 20 and 30 (e.g. 23 or 24).

In an Embodiment, R² or/and R³ comprises:

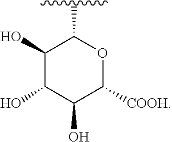 [SMILES: *[C@@H]1O[C@H](C(=O)O)[C@@H](O)[C@H](O)[C@H]1O]

In an Embodiment, R² and R³ each independently comprises a polyethylene glycol of formula:

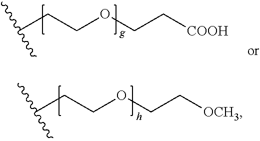

wherein g and h are independently an integer between 2 and 30.

In an Embodiment, D¹ and D² are the same.

In an Embodiment, D¹ and D² are different.

In an Embodiment, D¹ and D² are each independently selected from a cytotoxic drug, a cytostatic drug and an immunosuppressive drug.

In an Embodiment, D¹ and D² are each independently selected from an auristatin, a camptothecin, a duocarmycin, an etoposide, a maytansine, a maytansinoid, a taxane, a benzodiazepine or benzodiazepine containing drug (e.g., pyrrolo[1,4]-benzodiazepines (PBDs), indolinobenzodiazepines, and oxazolidinobenzodiazepines), and a vinca alkaloid.

In an embodiment, D¹ and D² are each independently selected from the group consisting of:

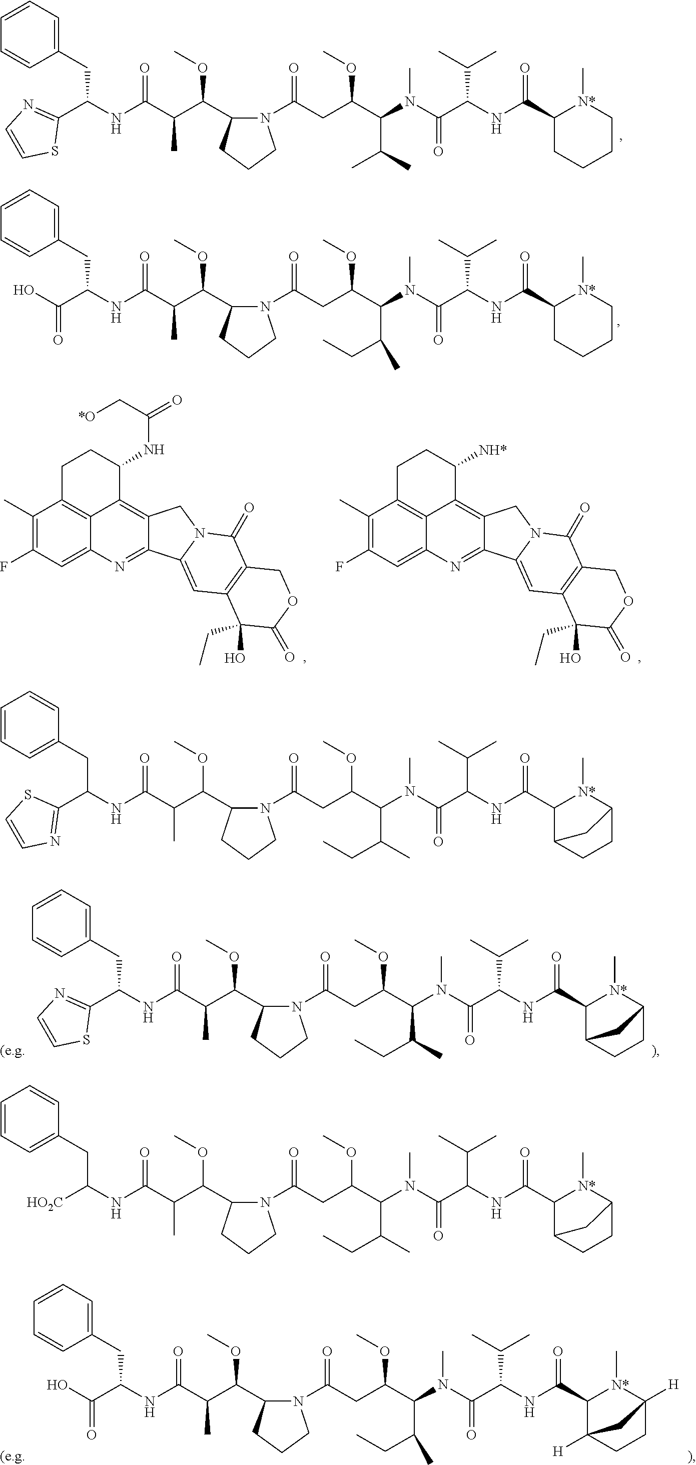

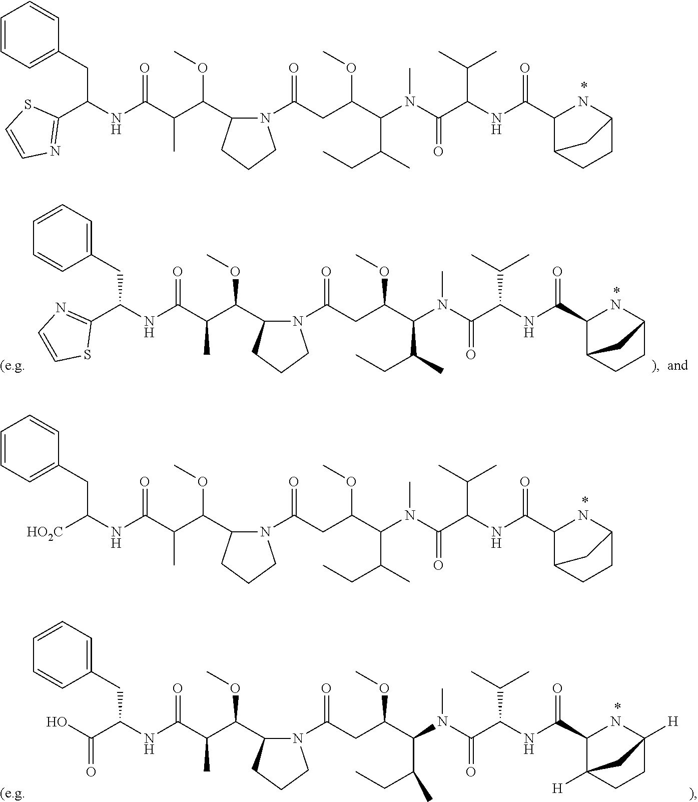

wherein * indicates the point of attachment to A¹ or A²; or a pharmaceutically acceptable salt thereof.

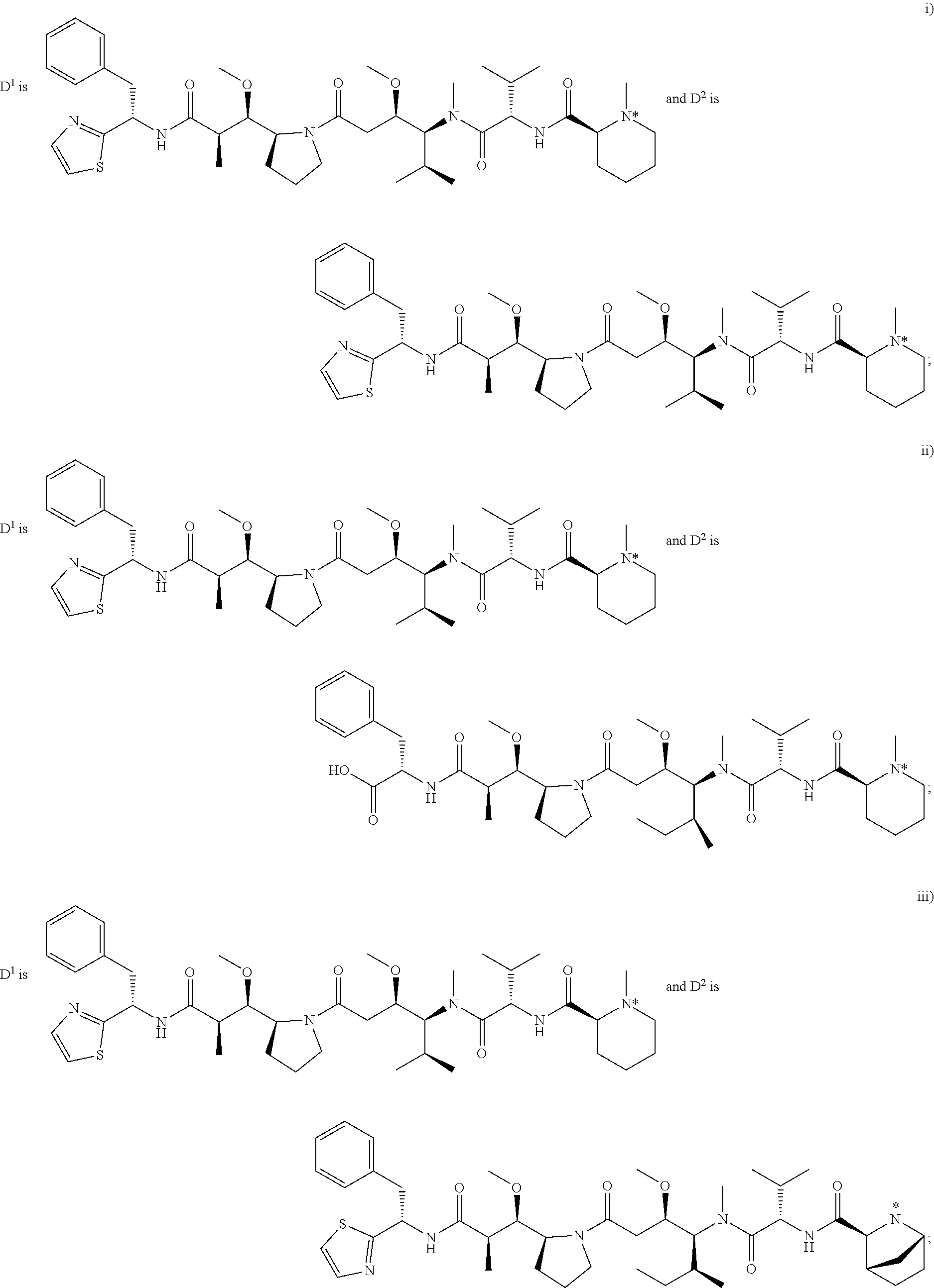

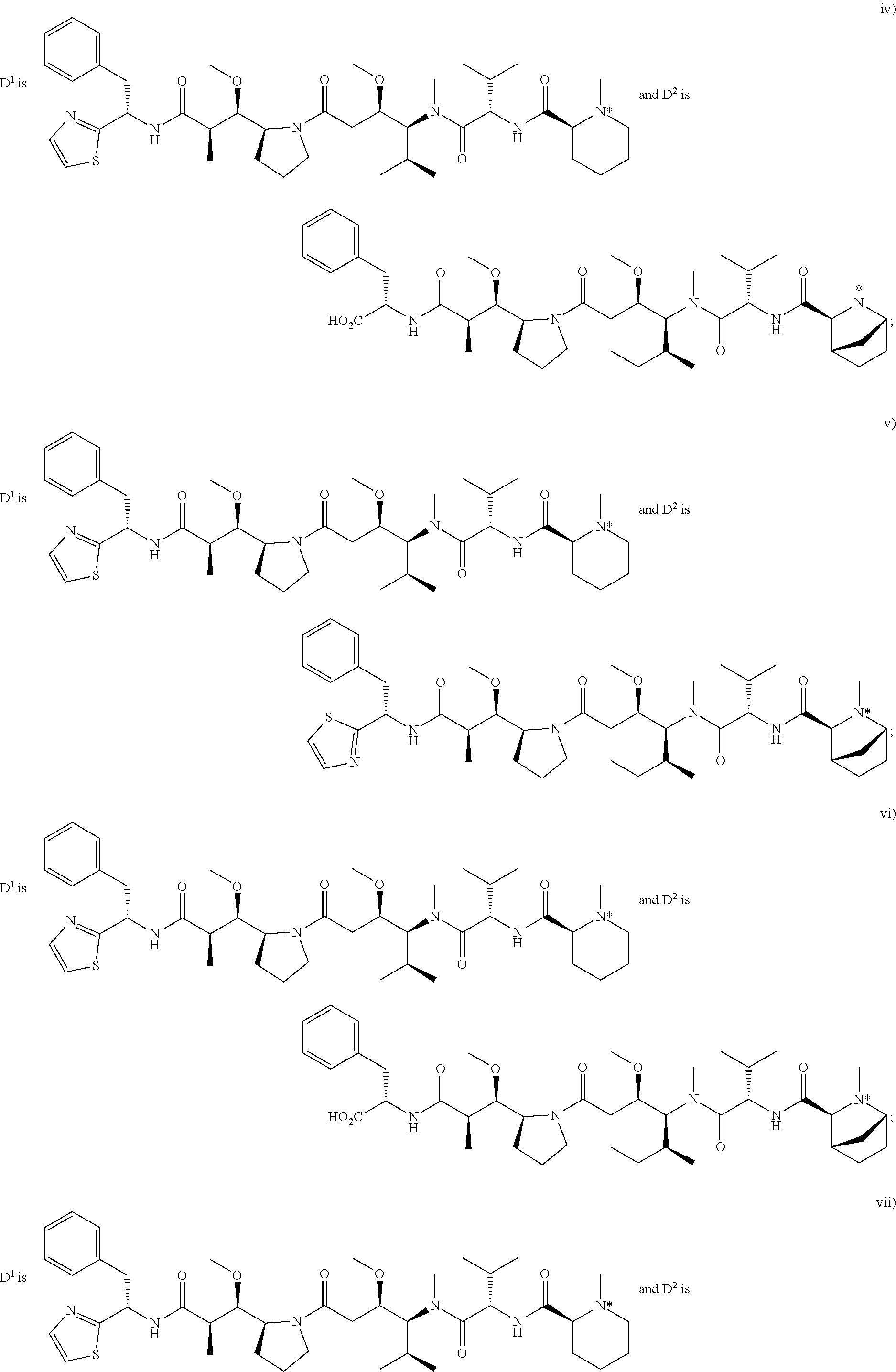

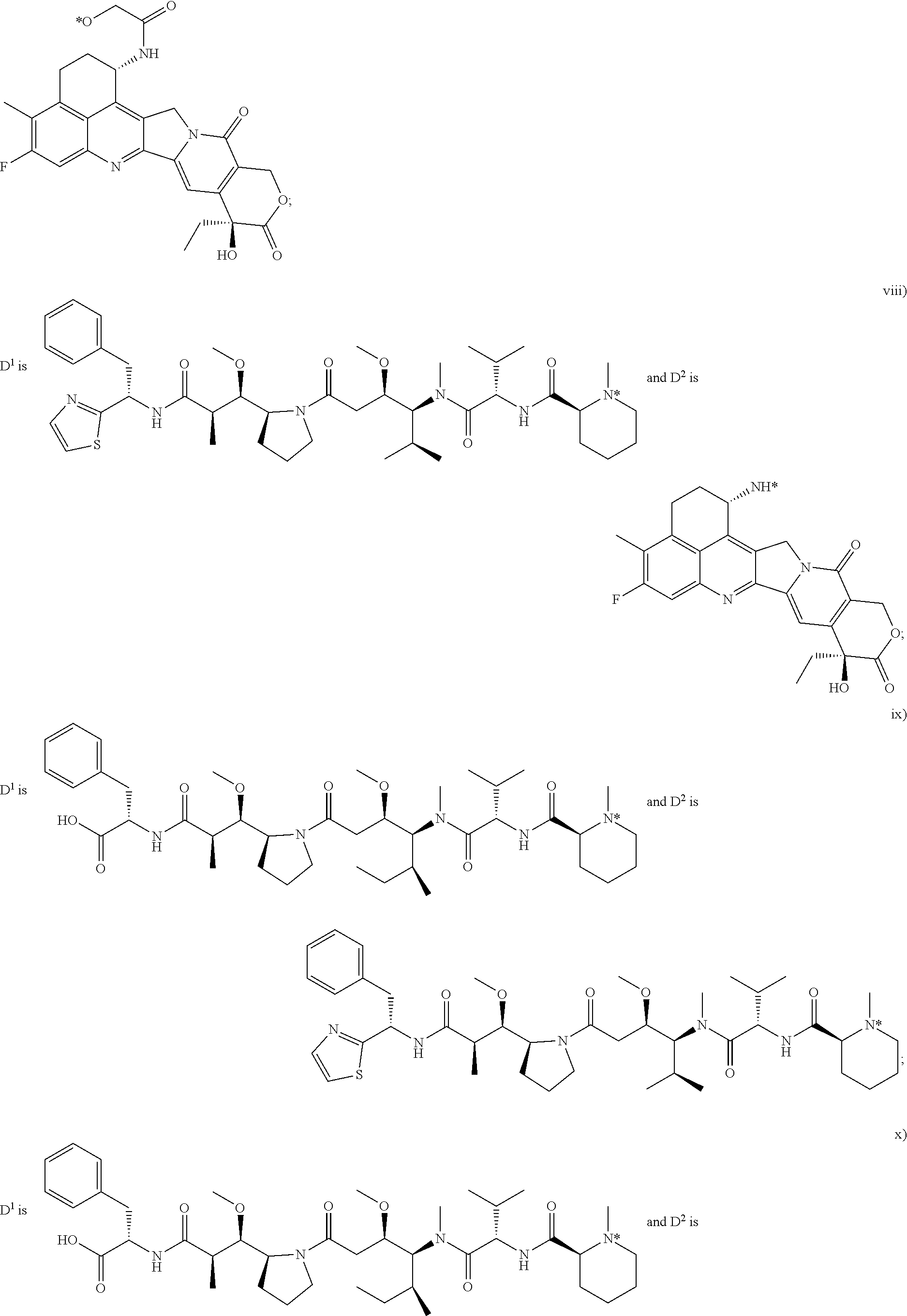

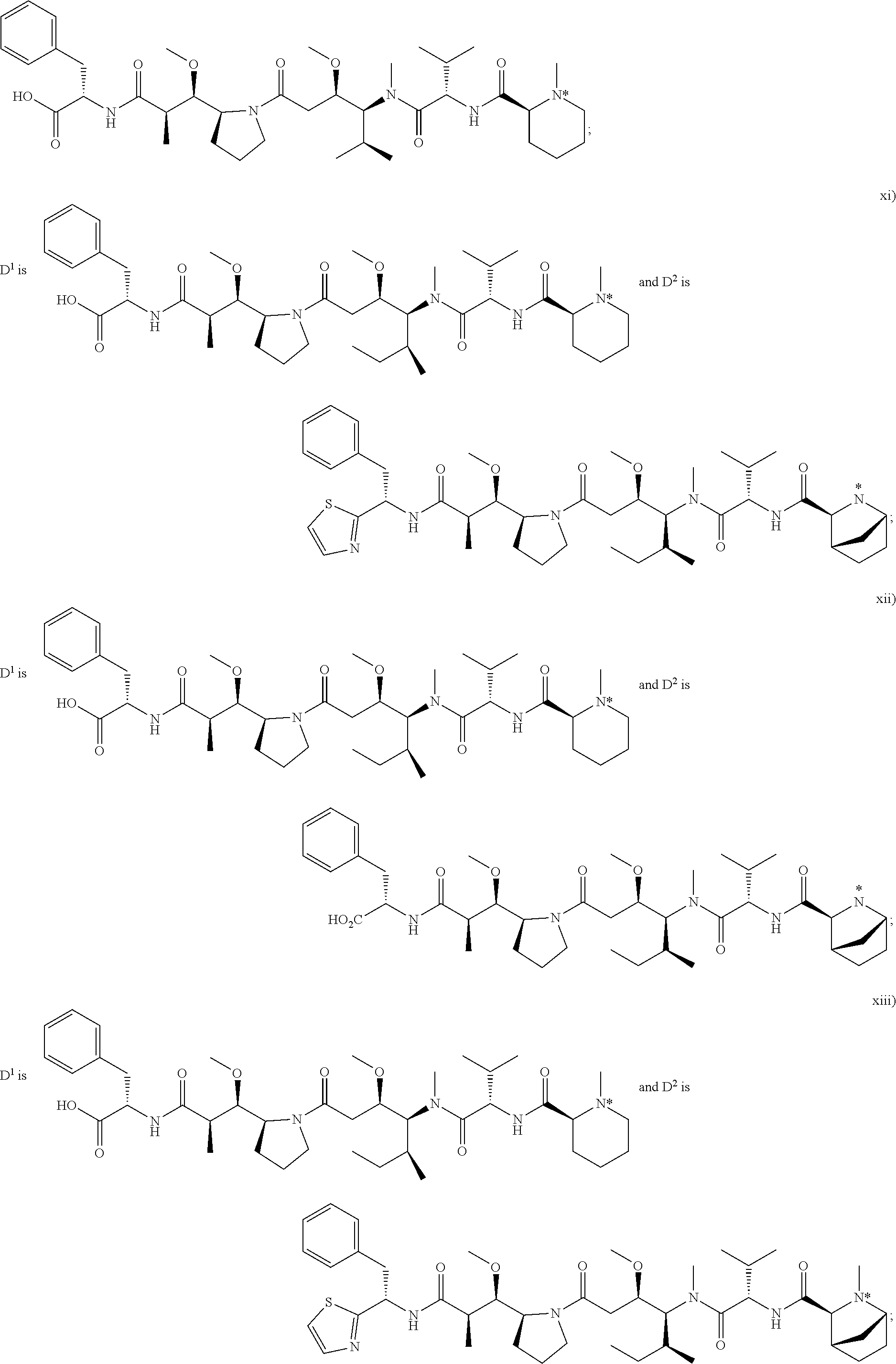

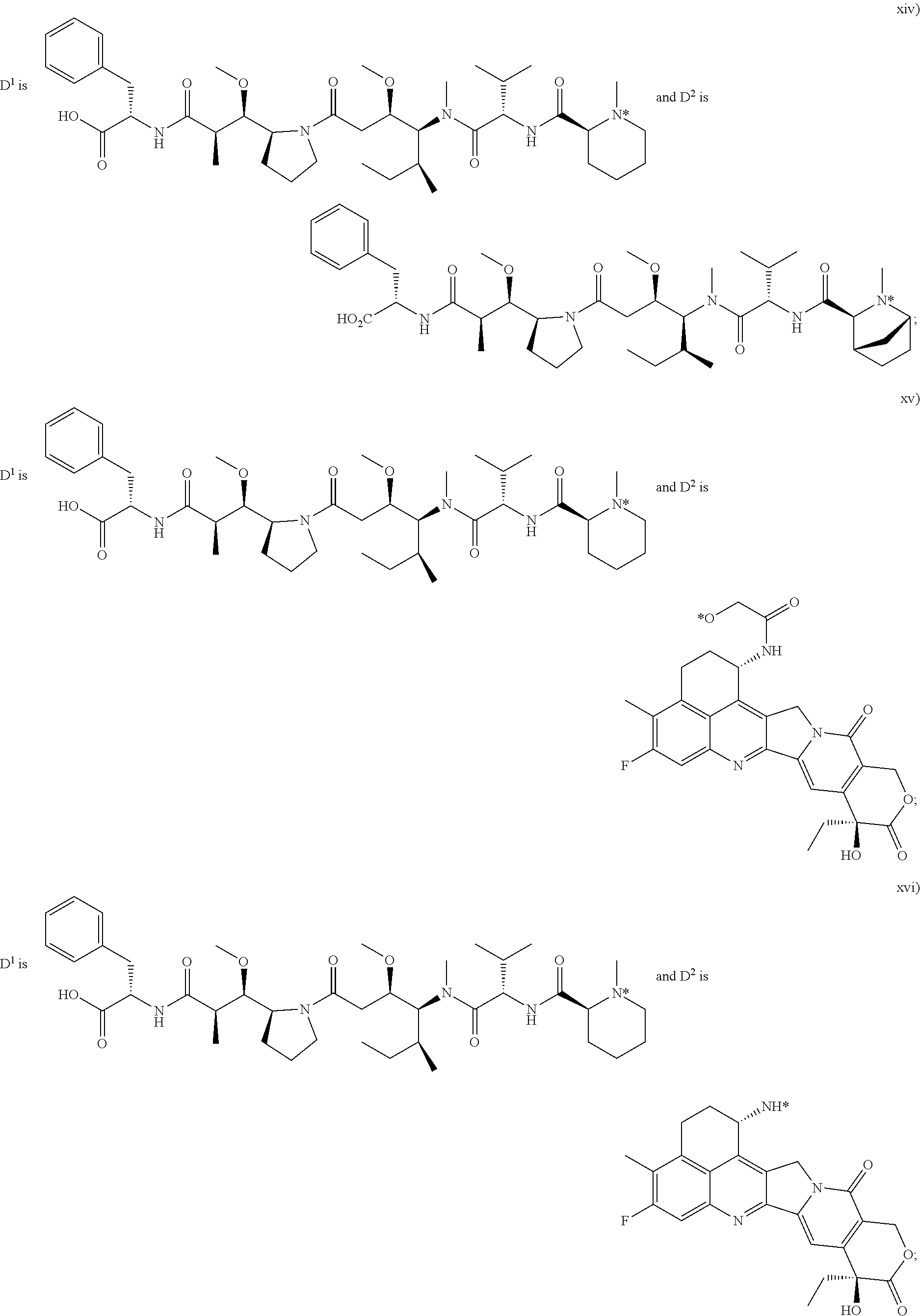

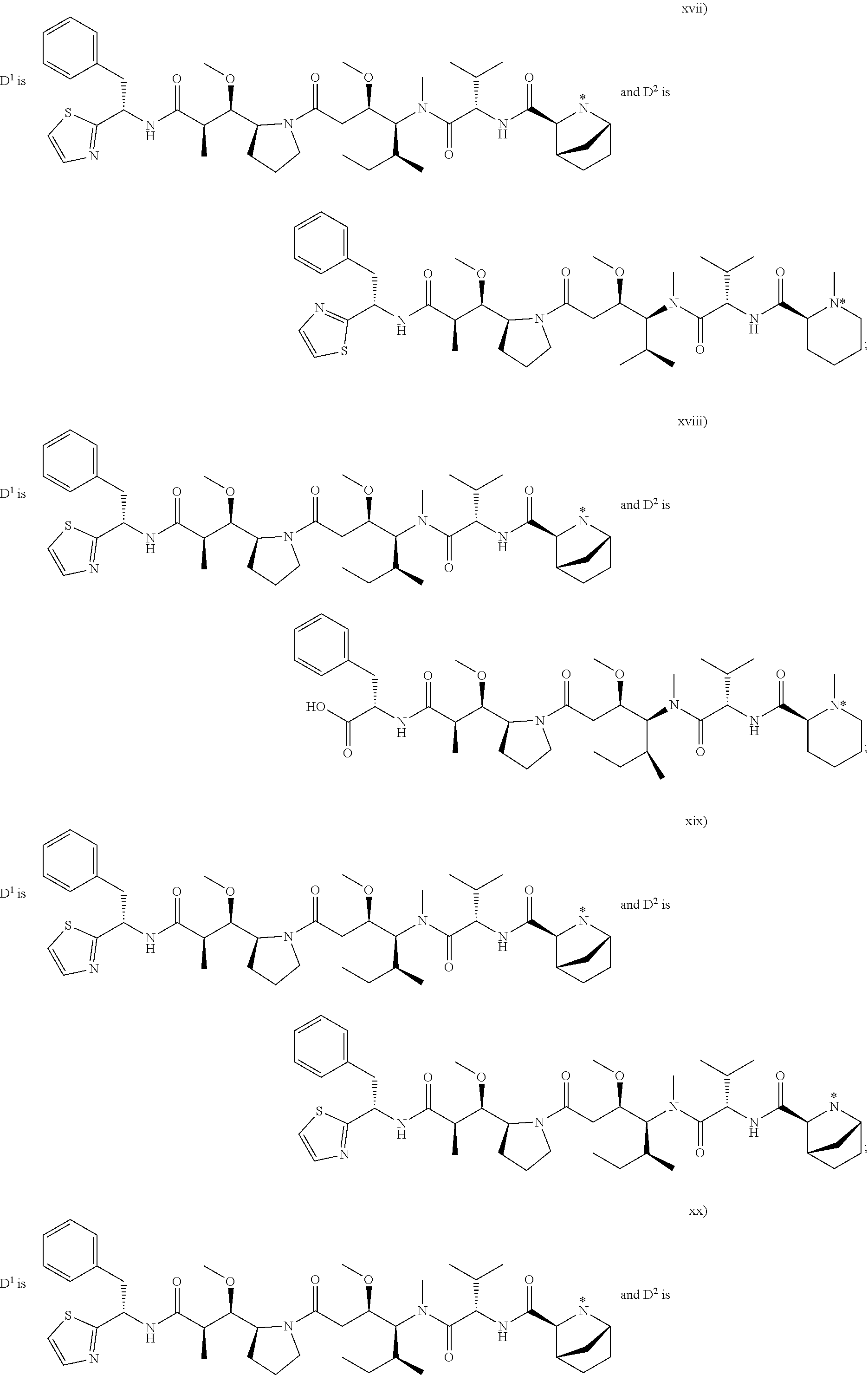

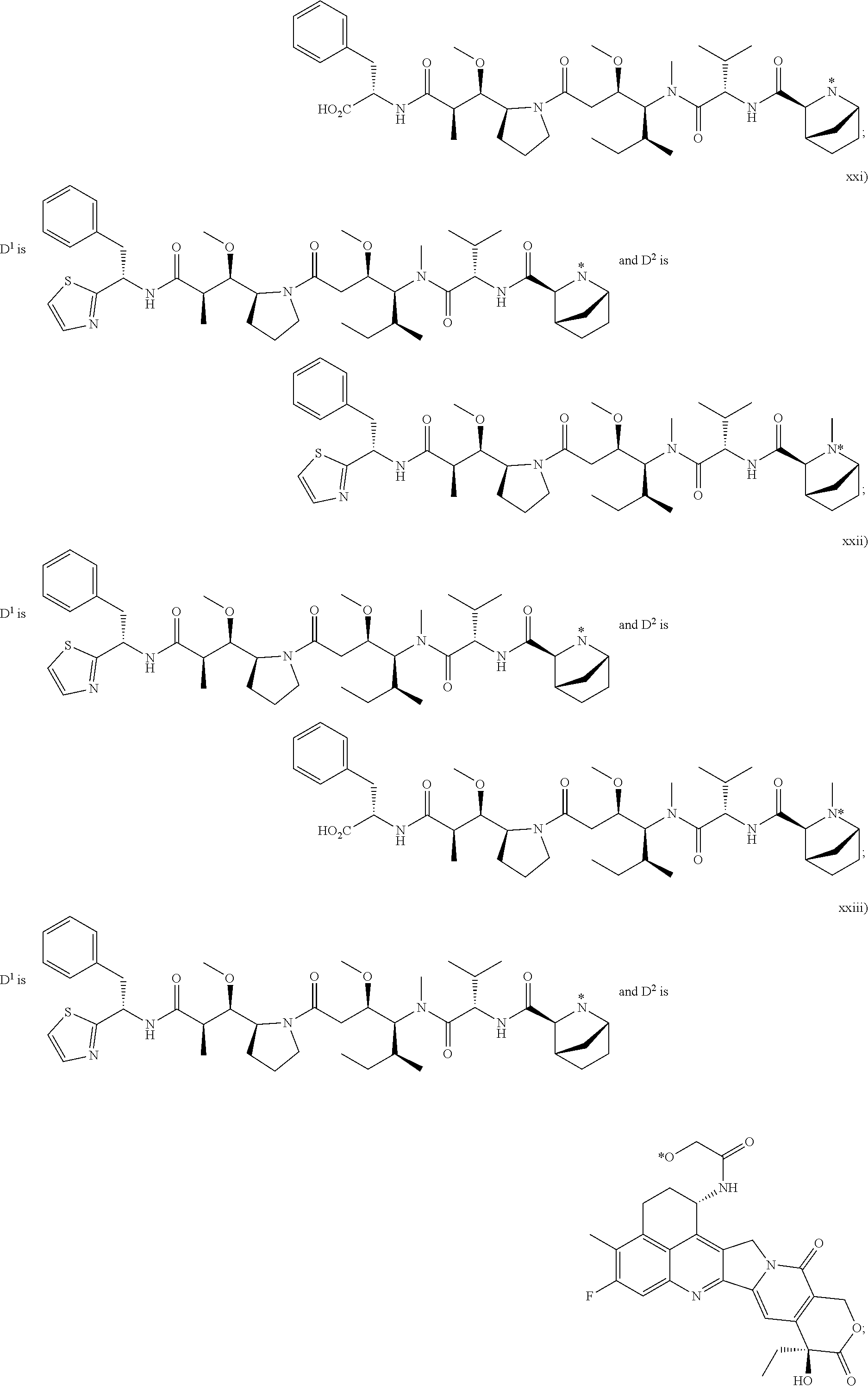

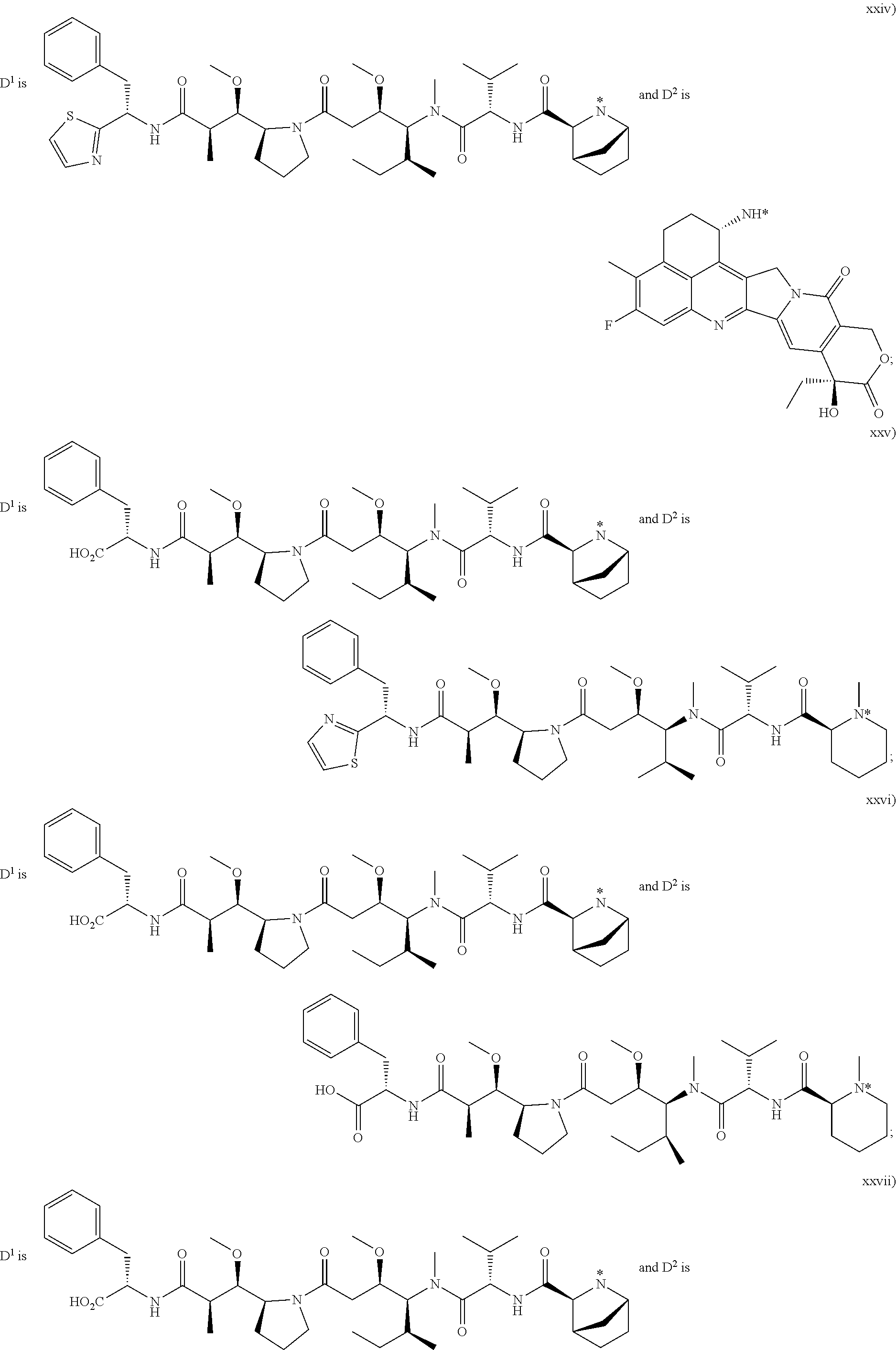

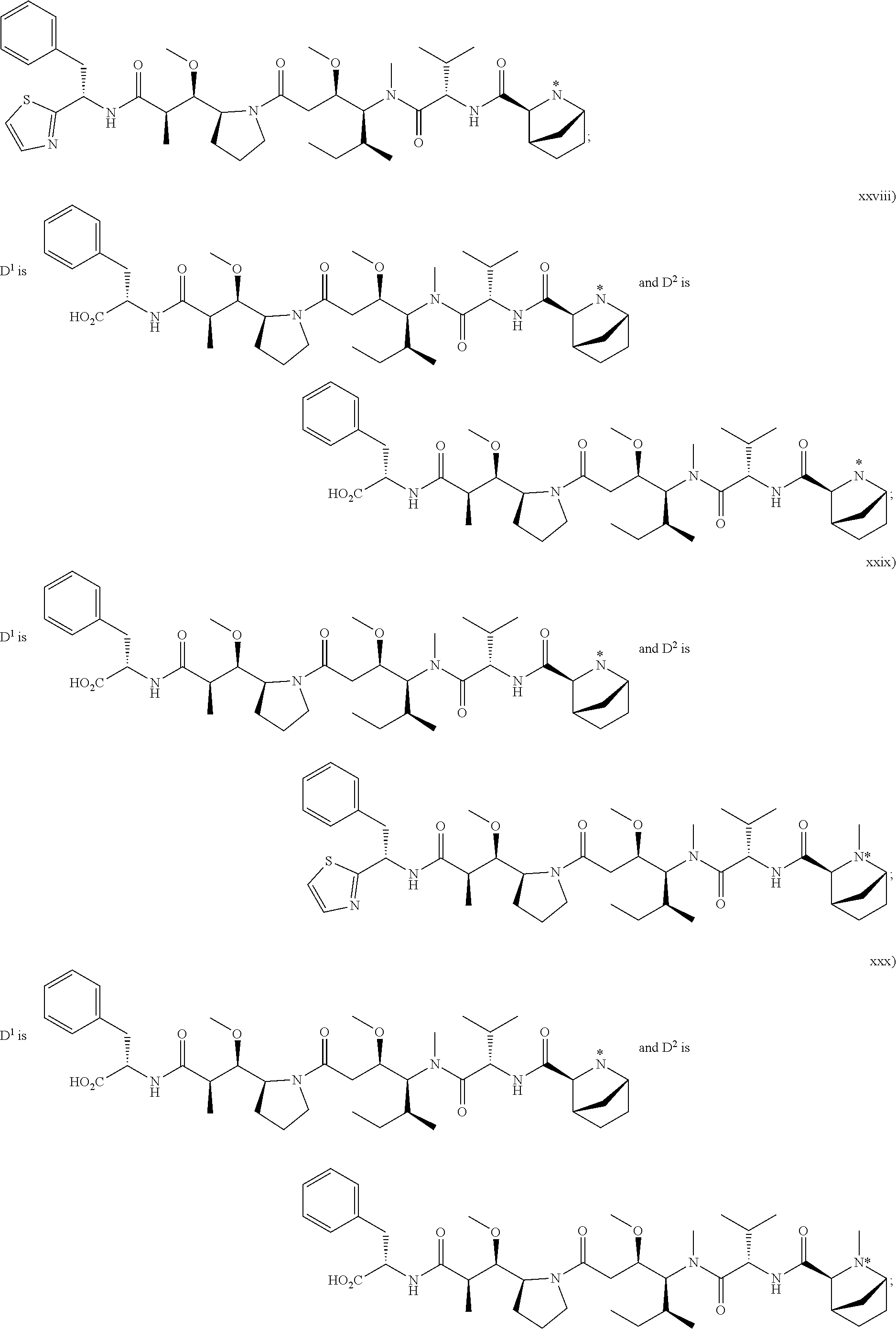

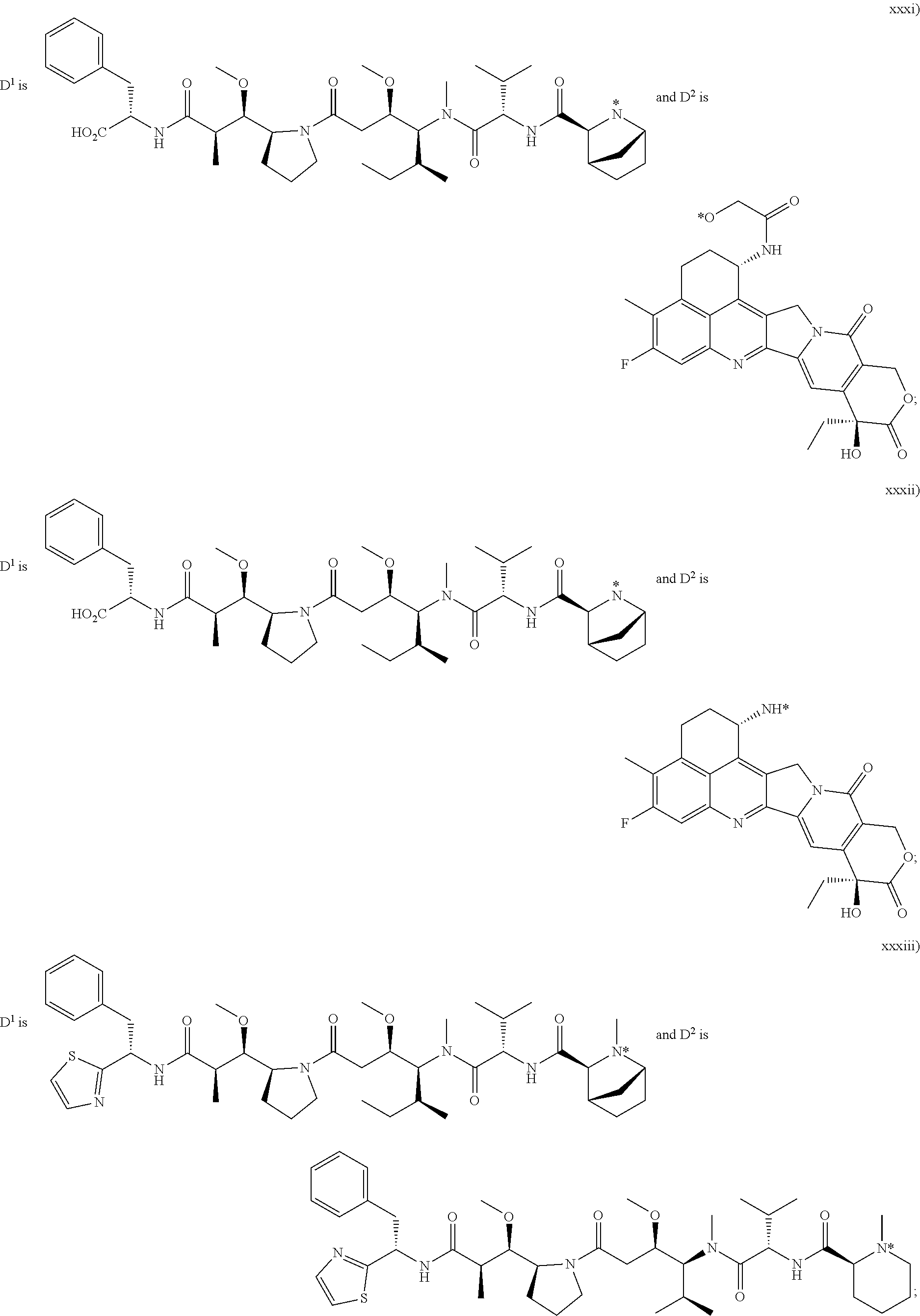

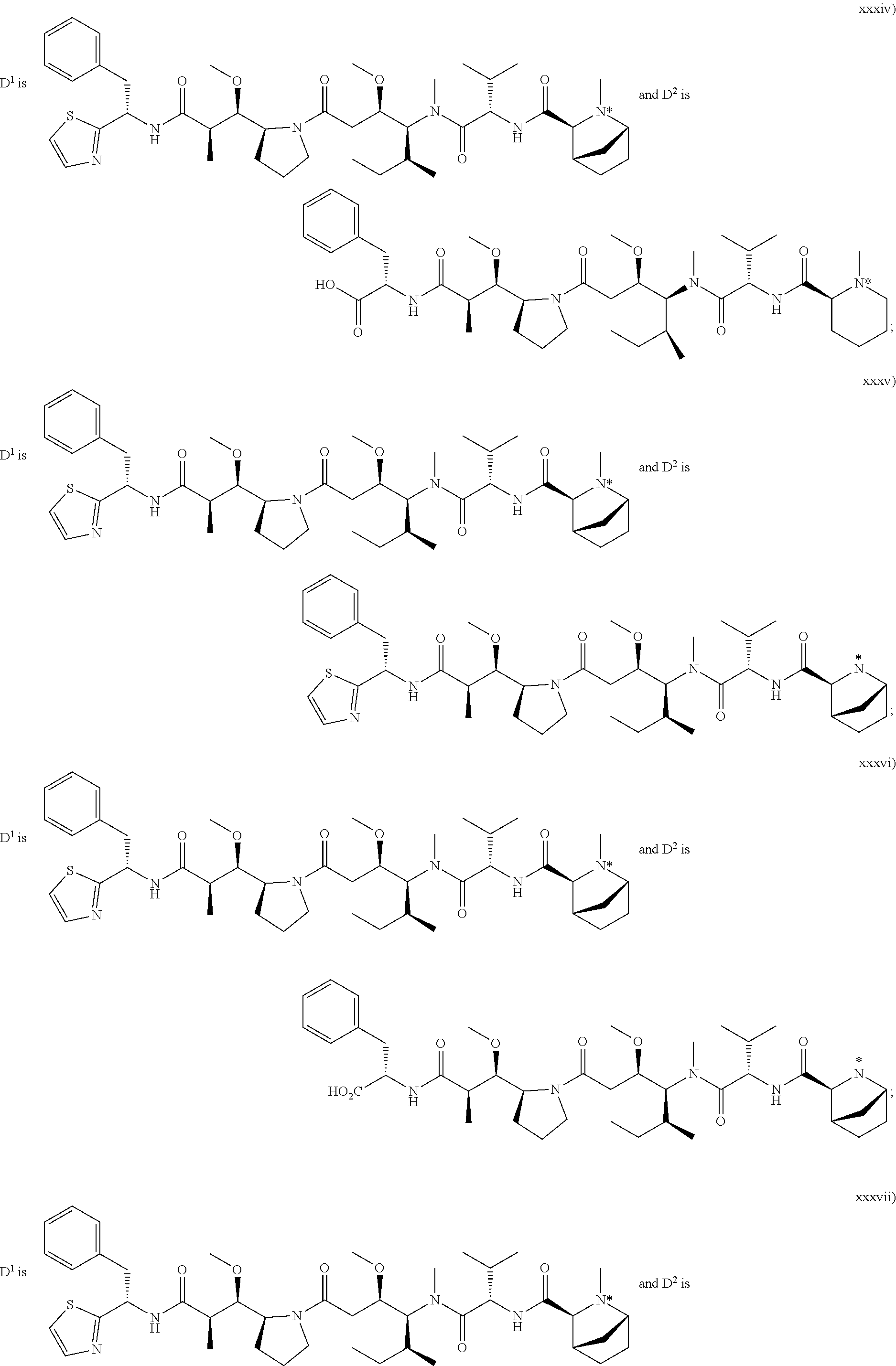

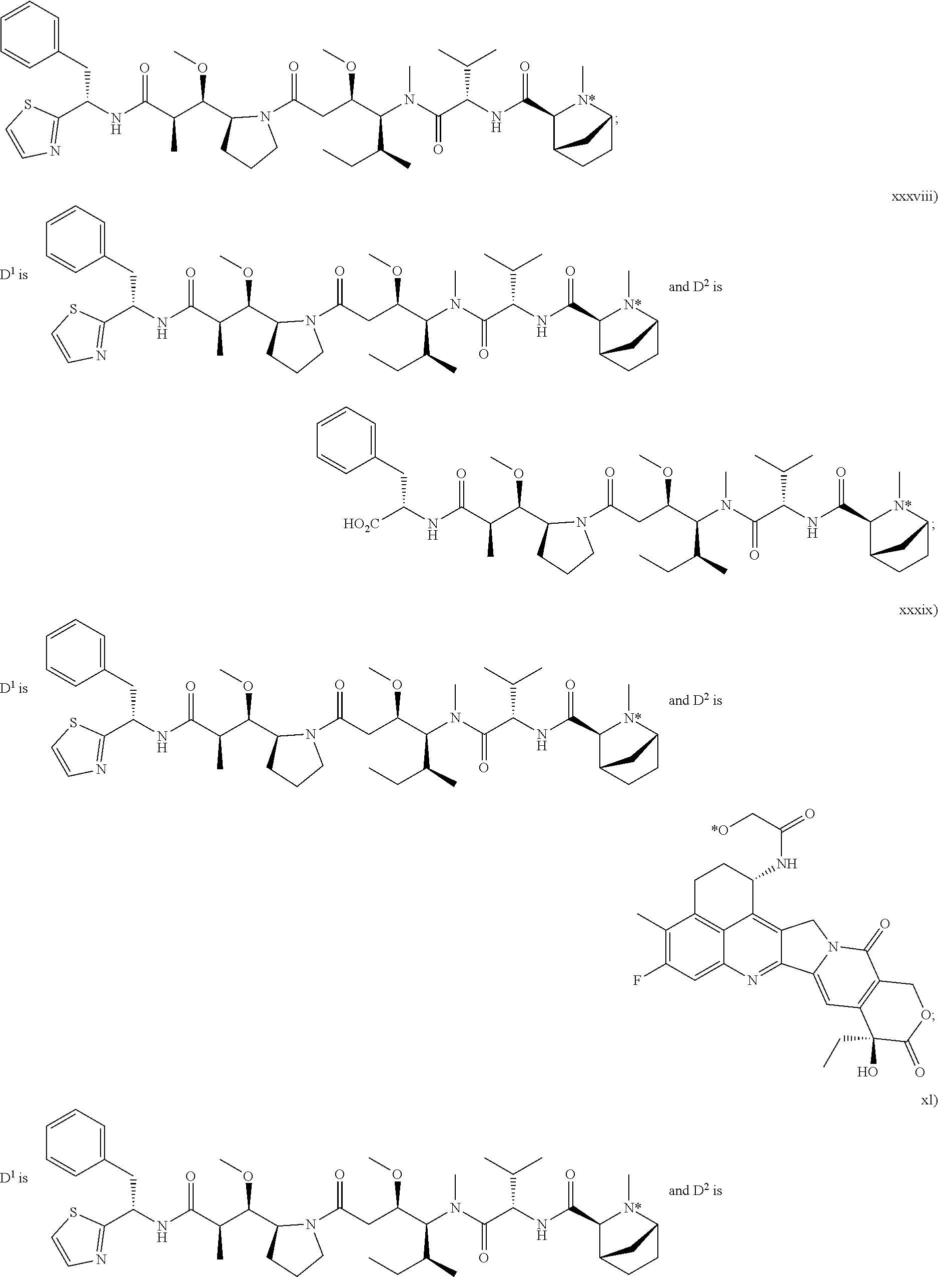

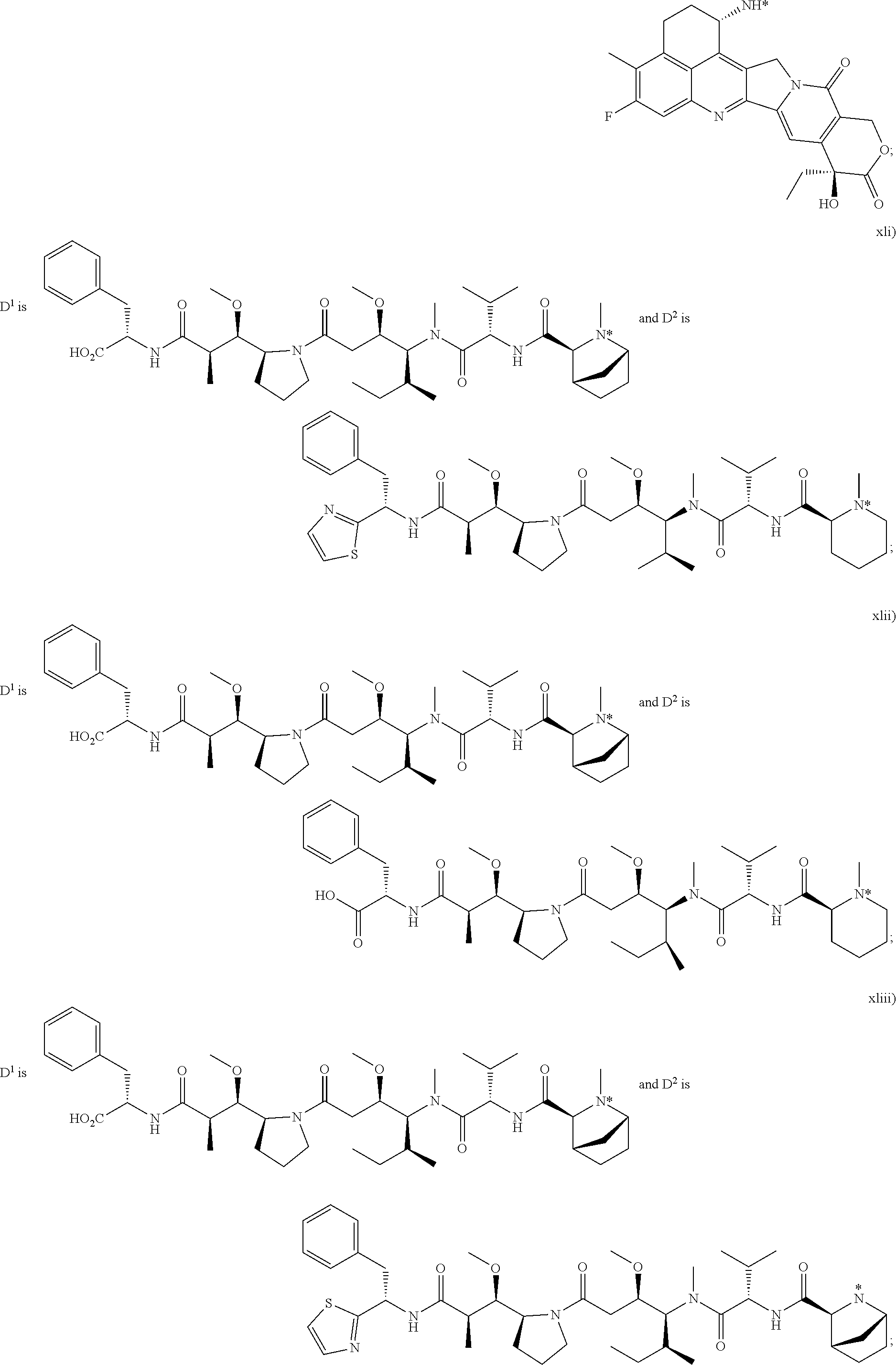

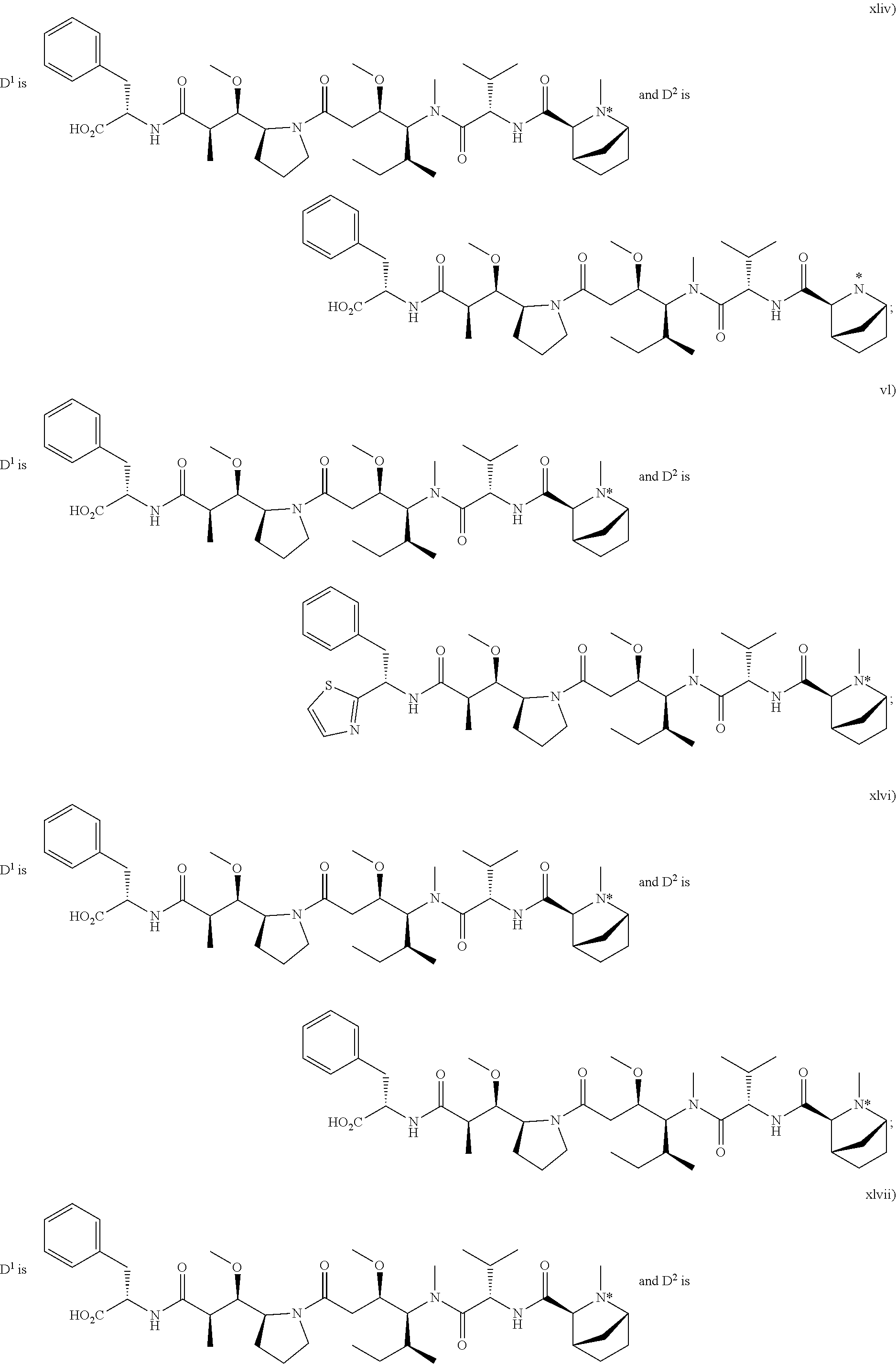

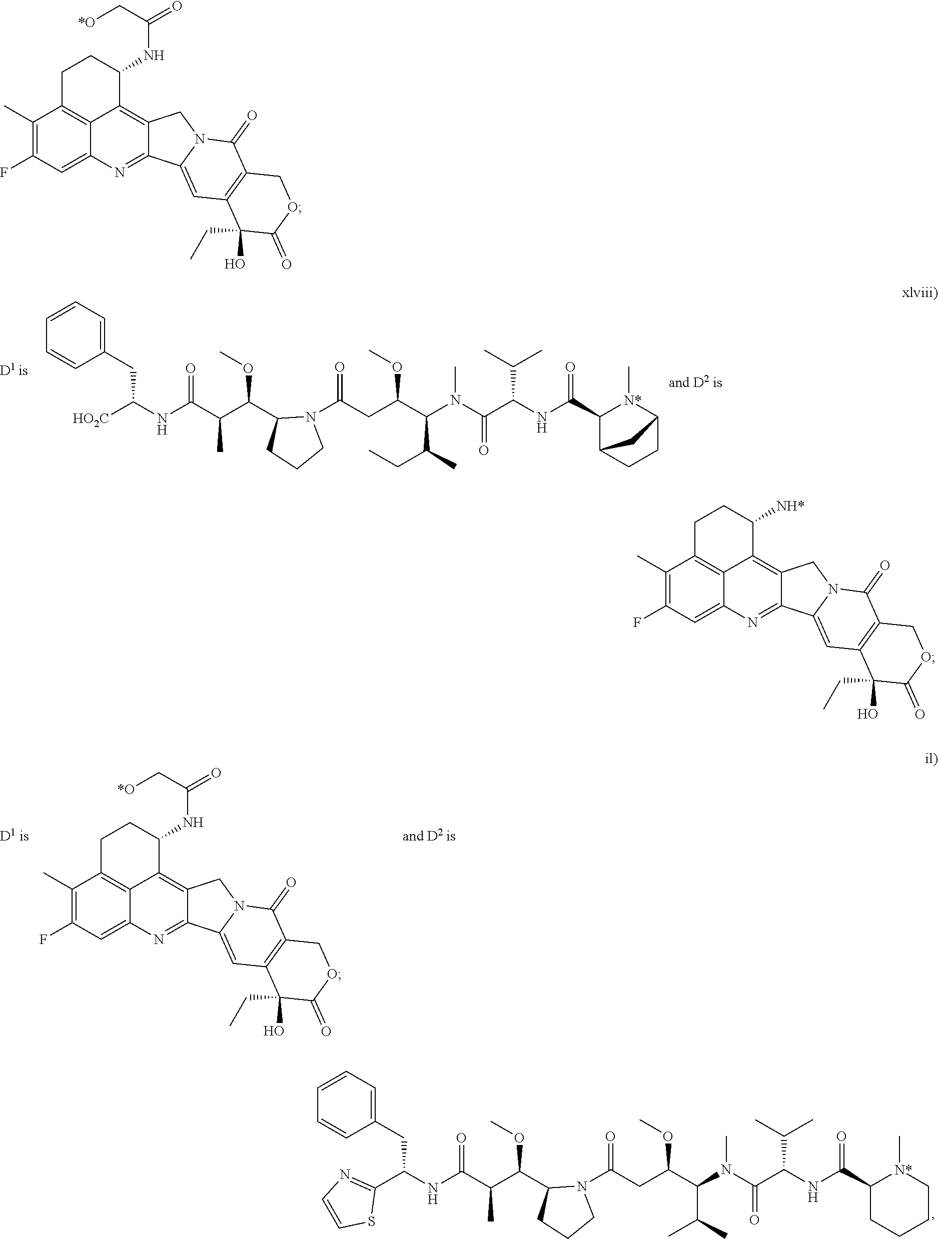

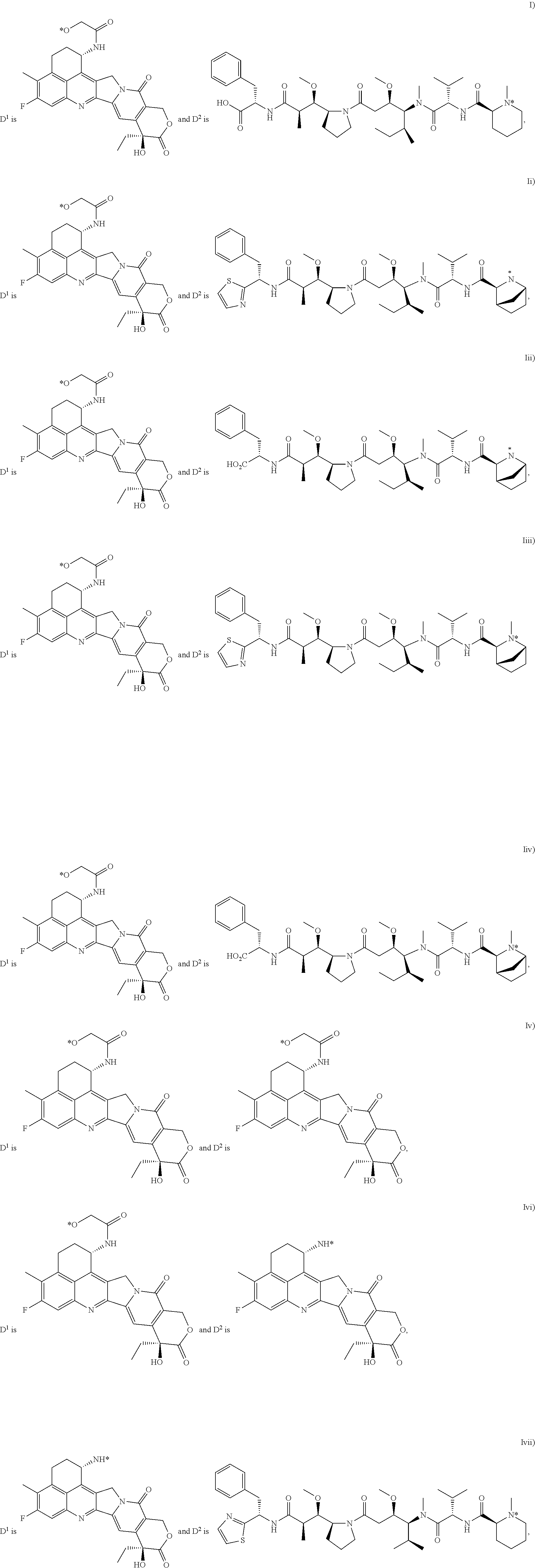

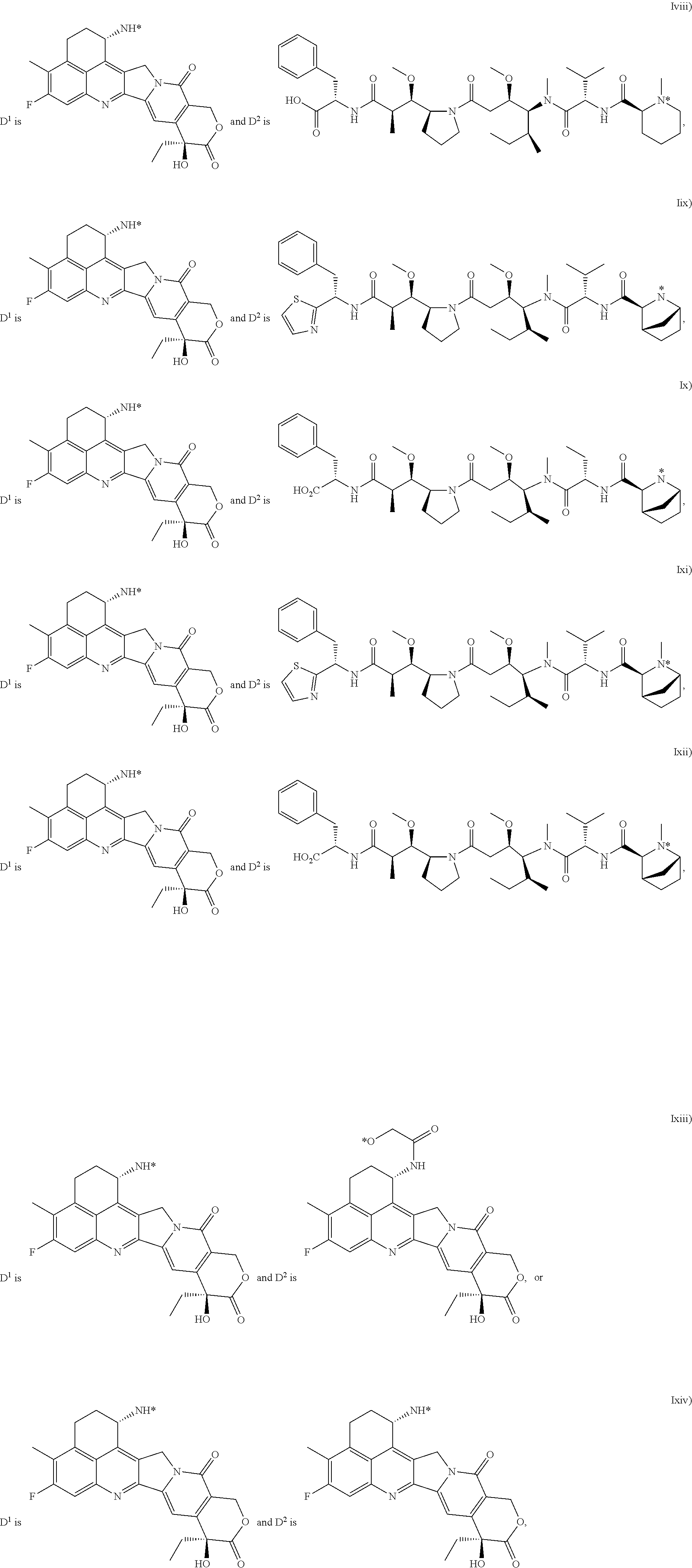

In an embodiment:

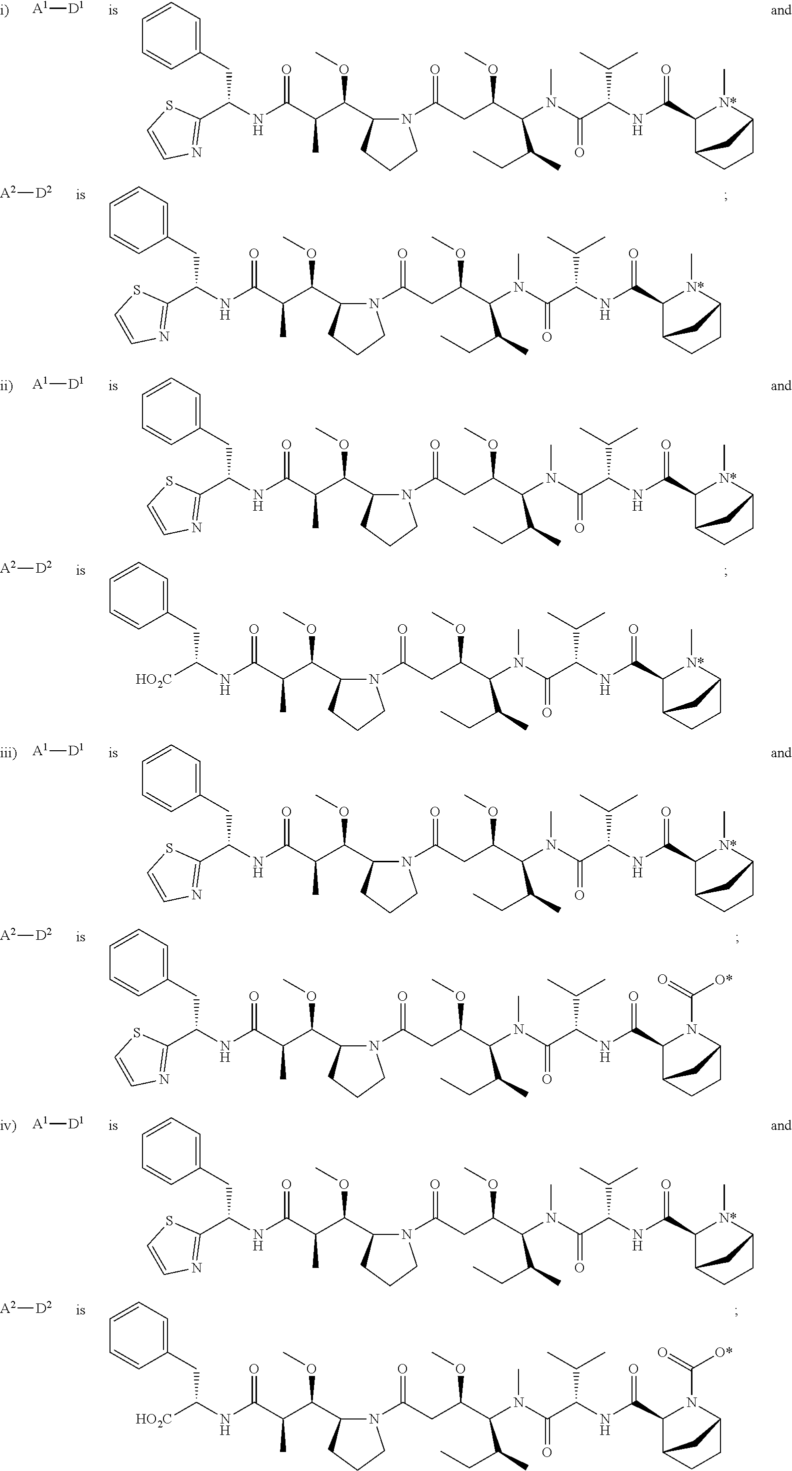

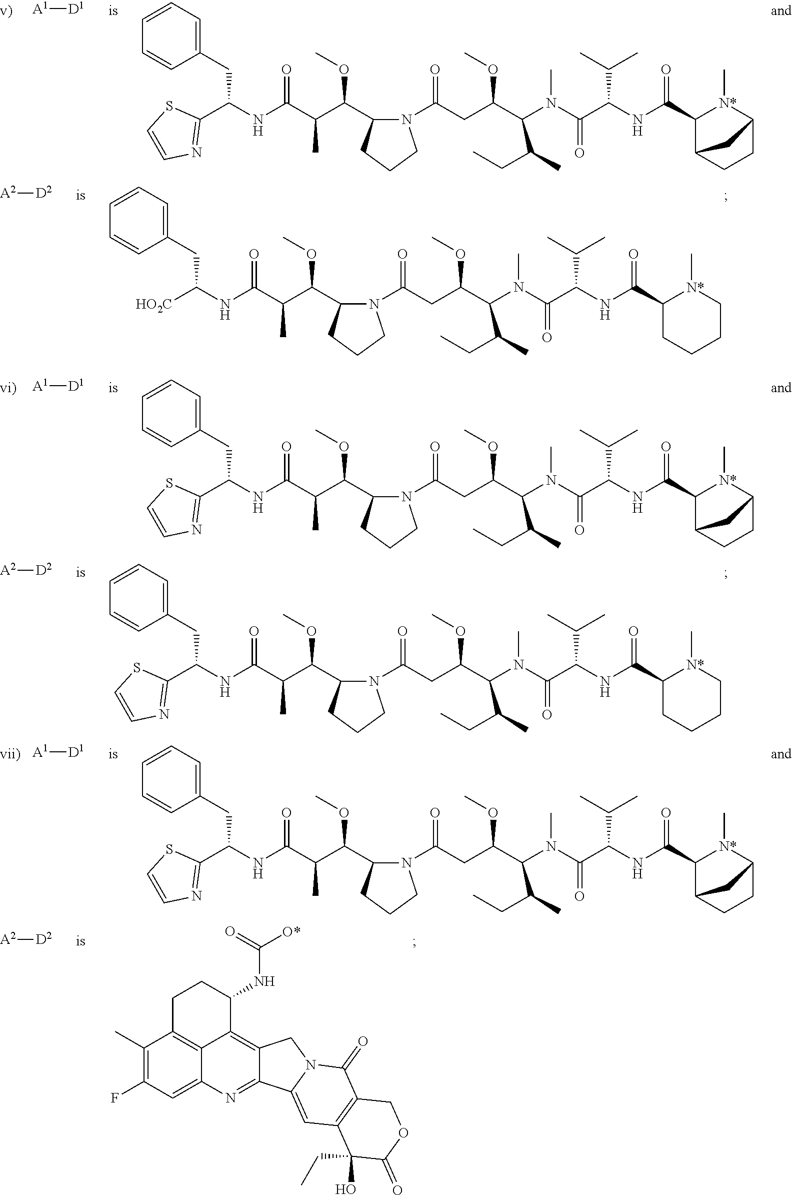

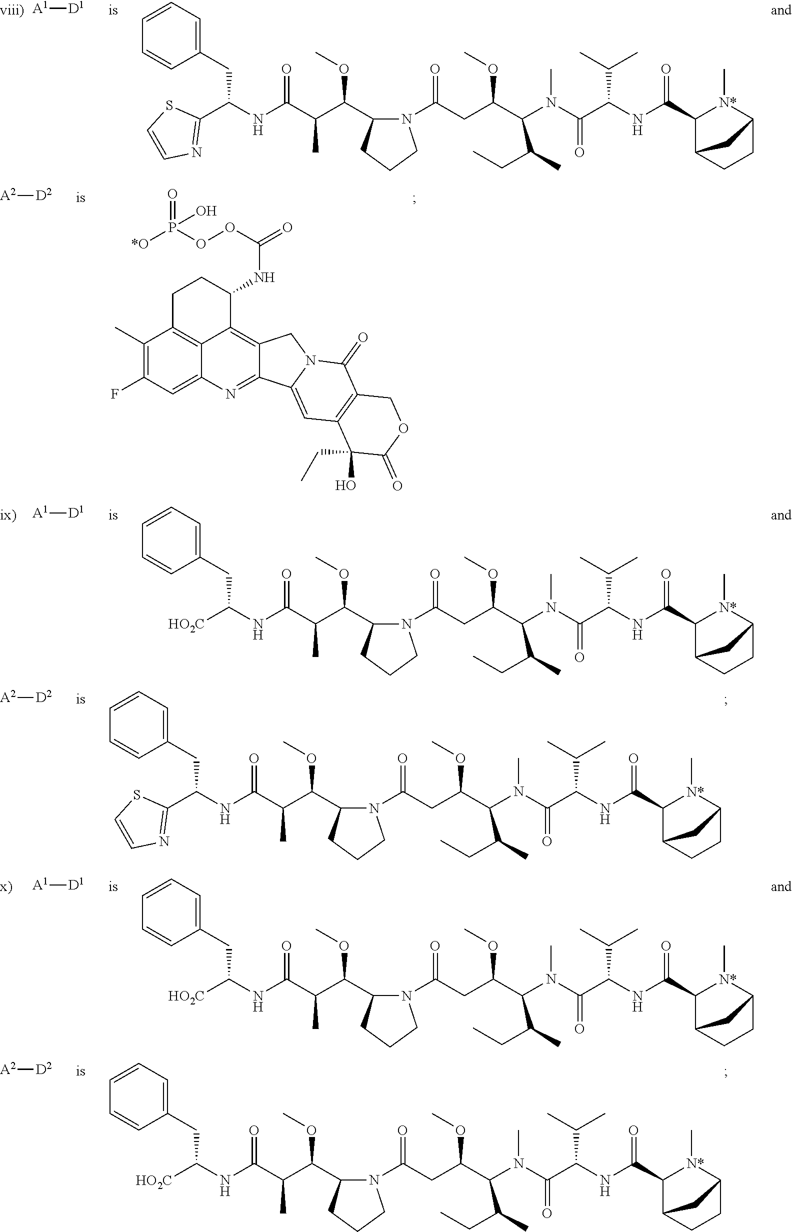

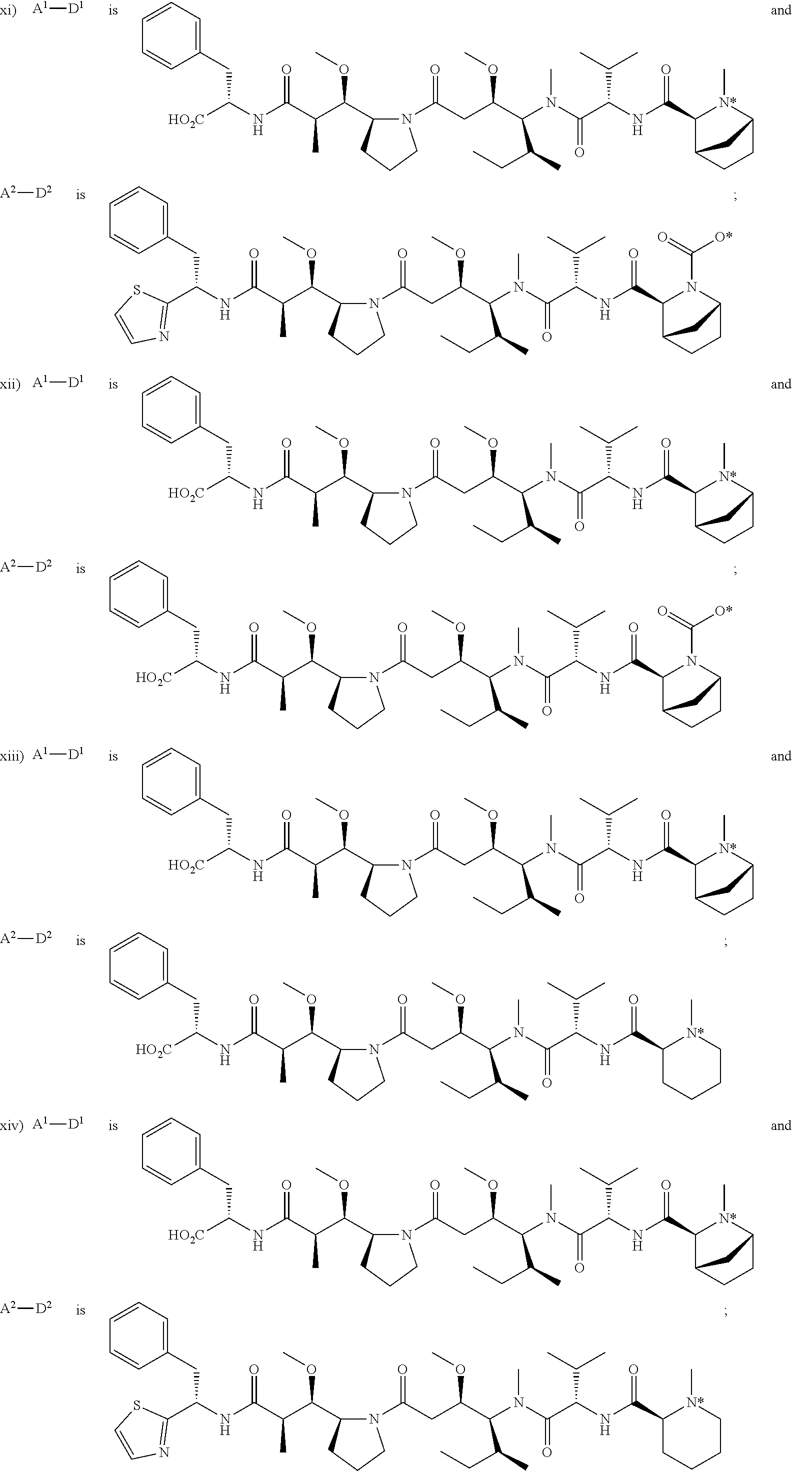

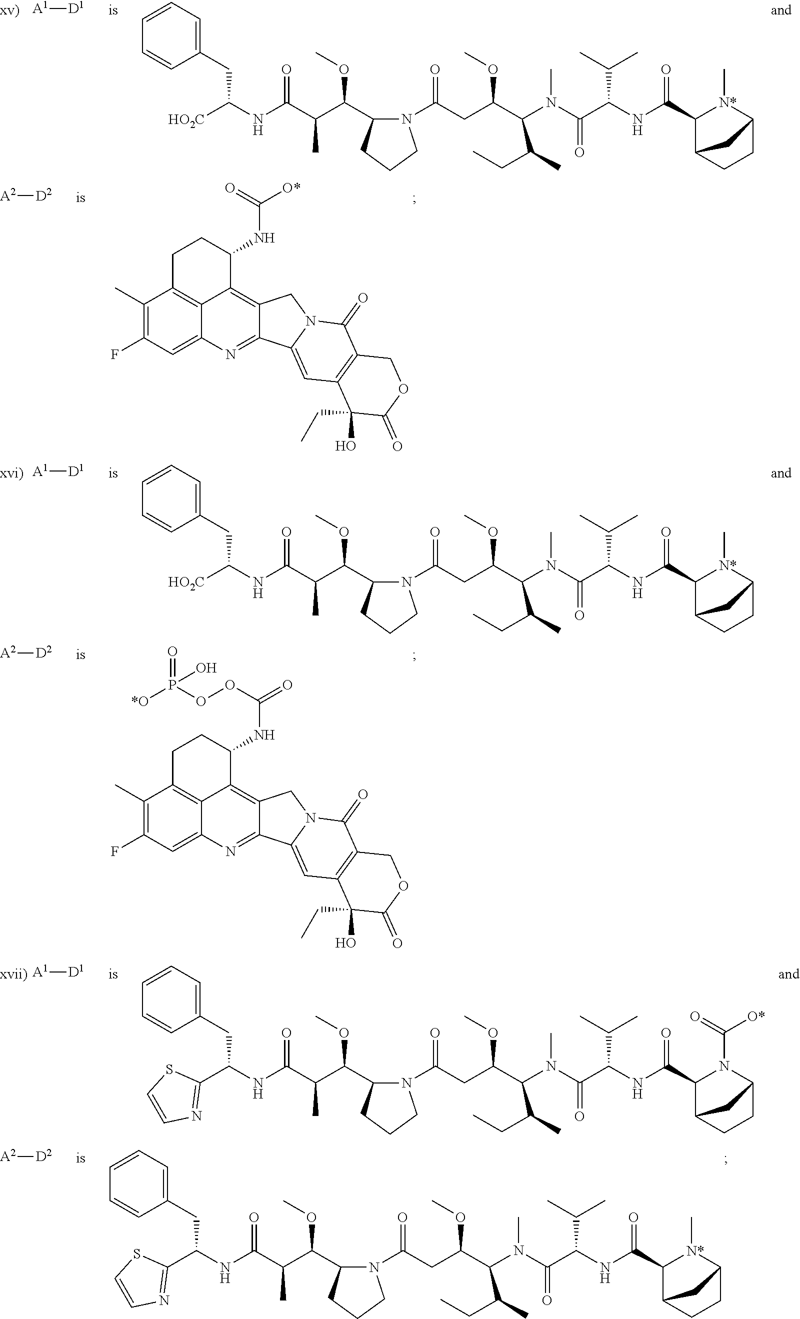

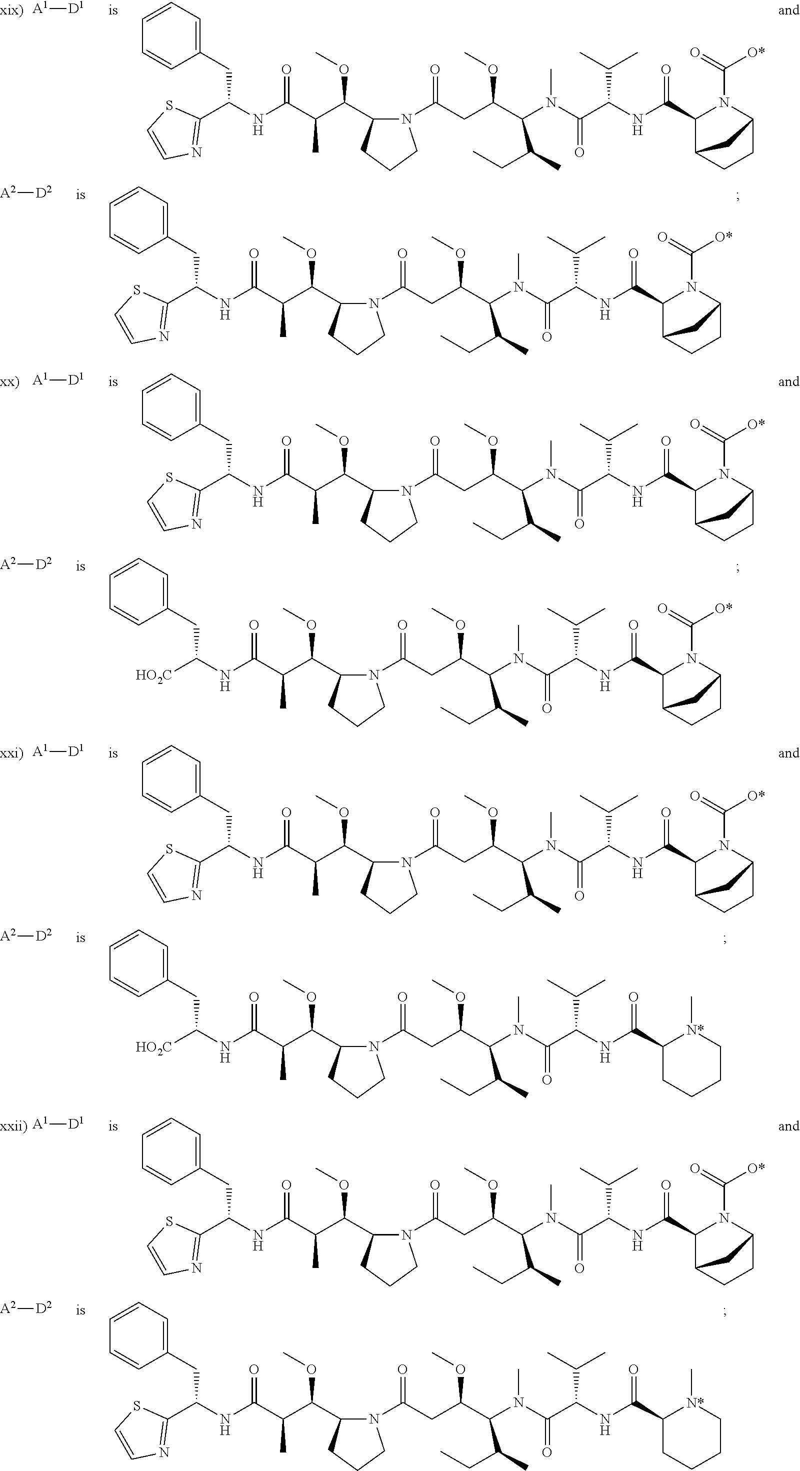

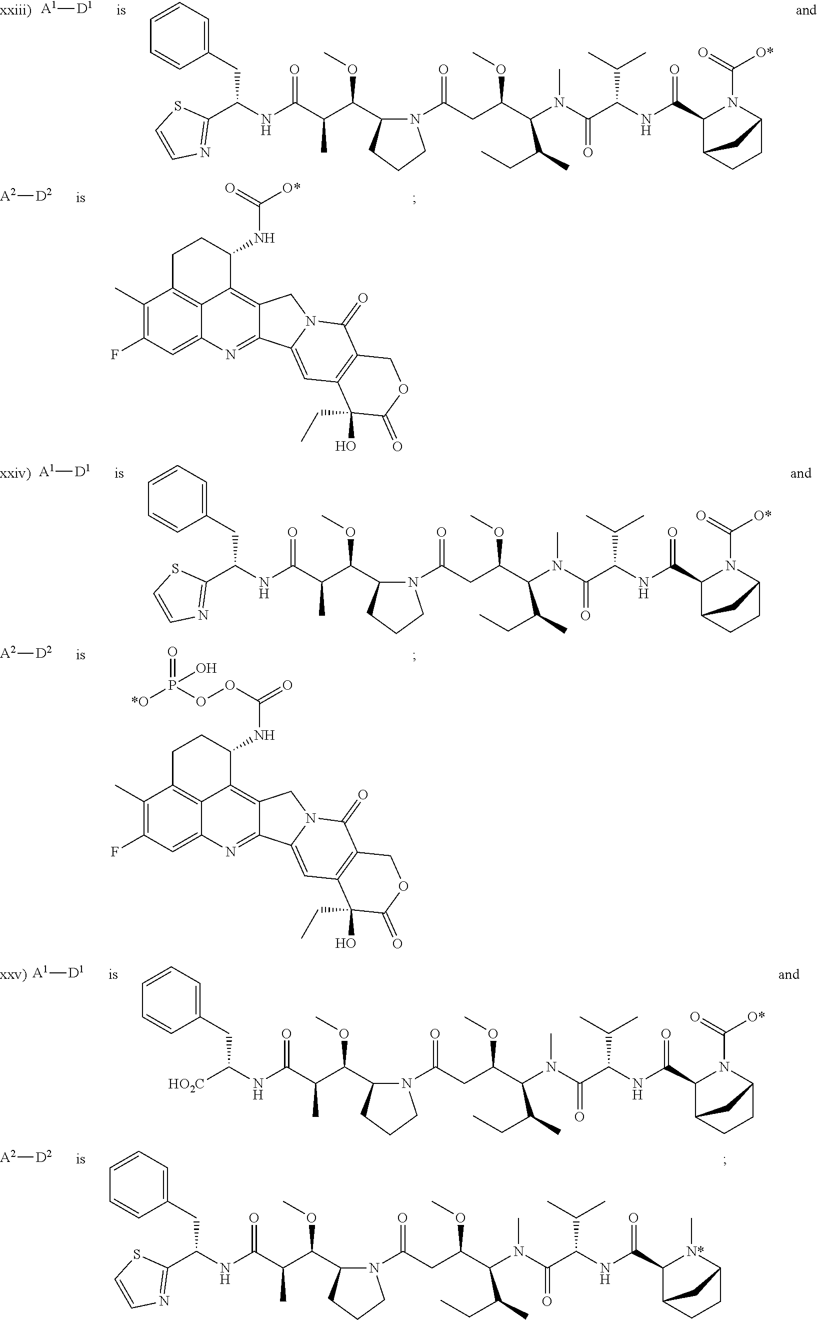

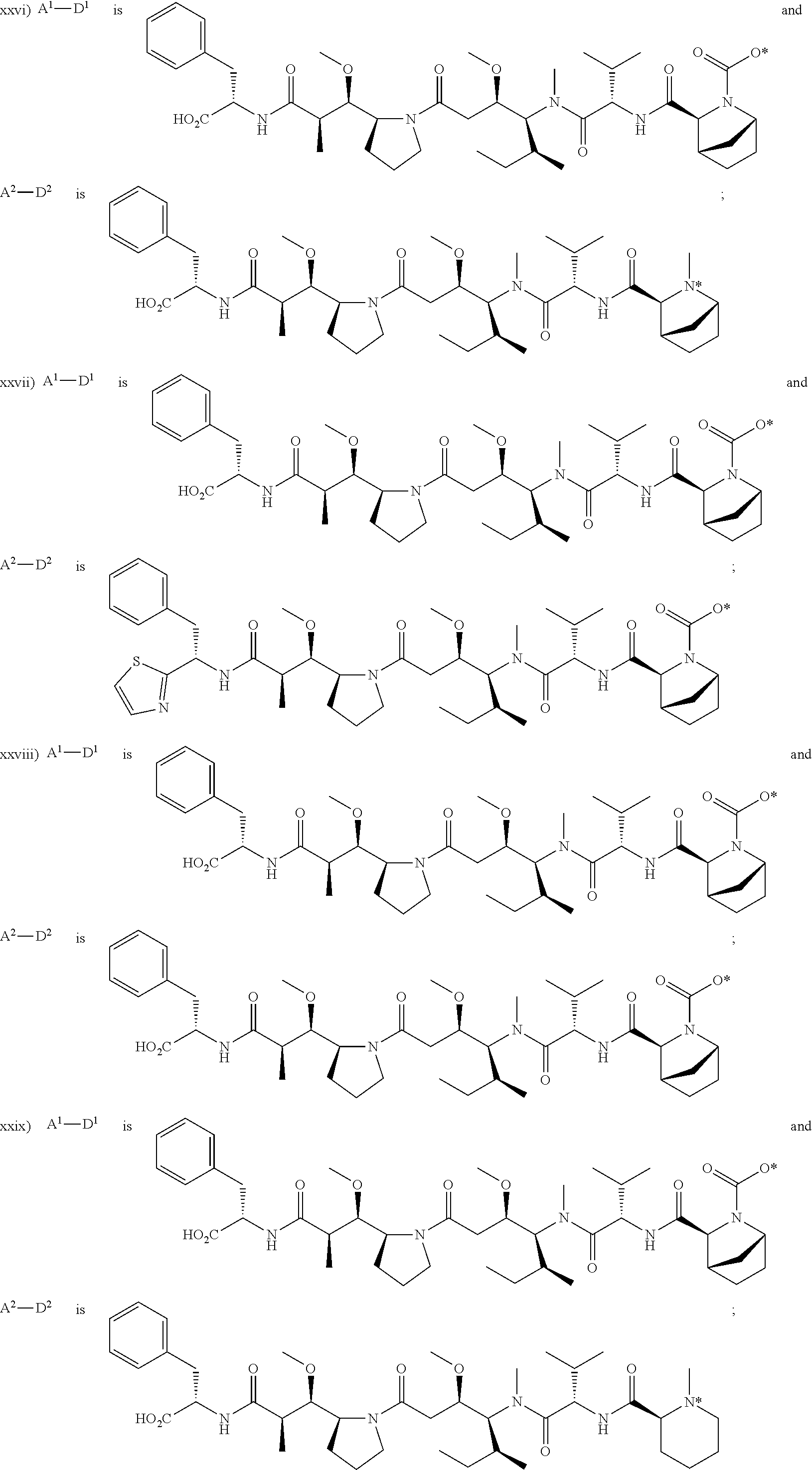

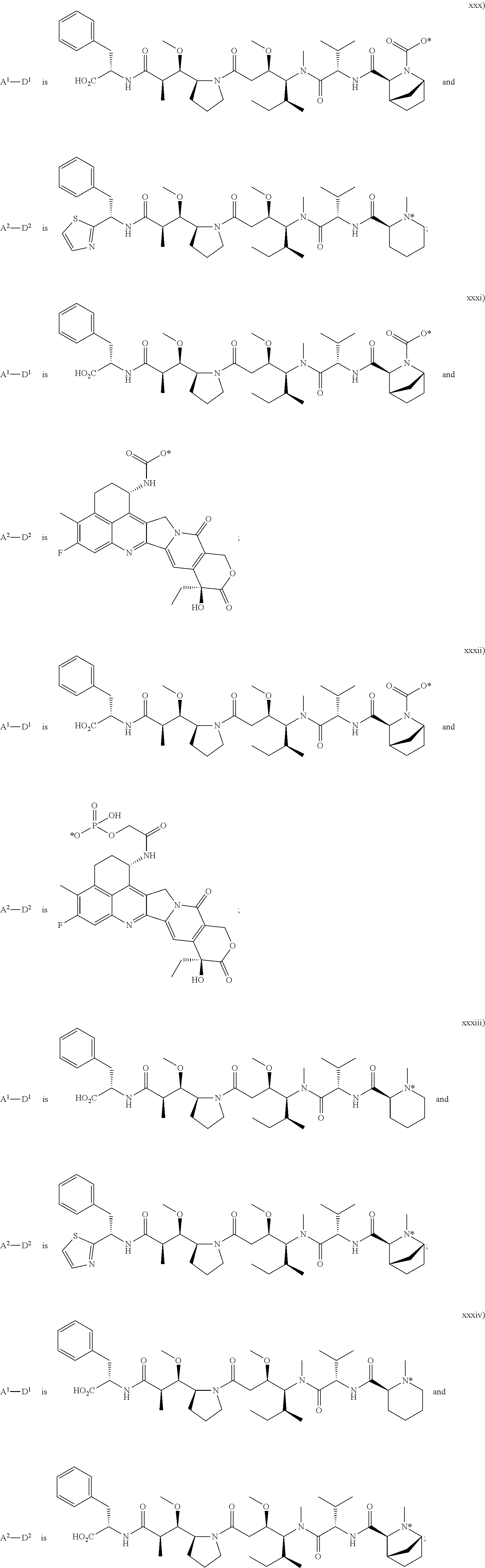

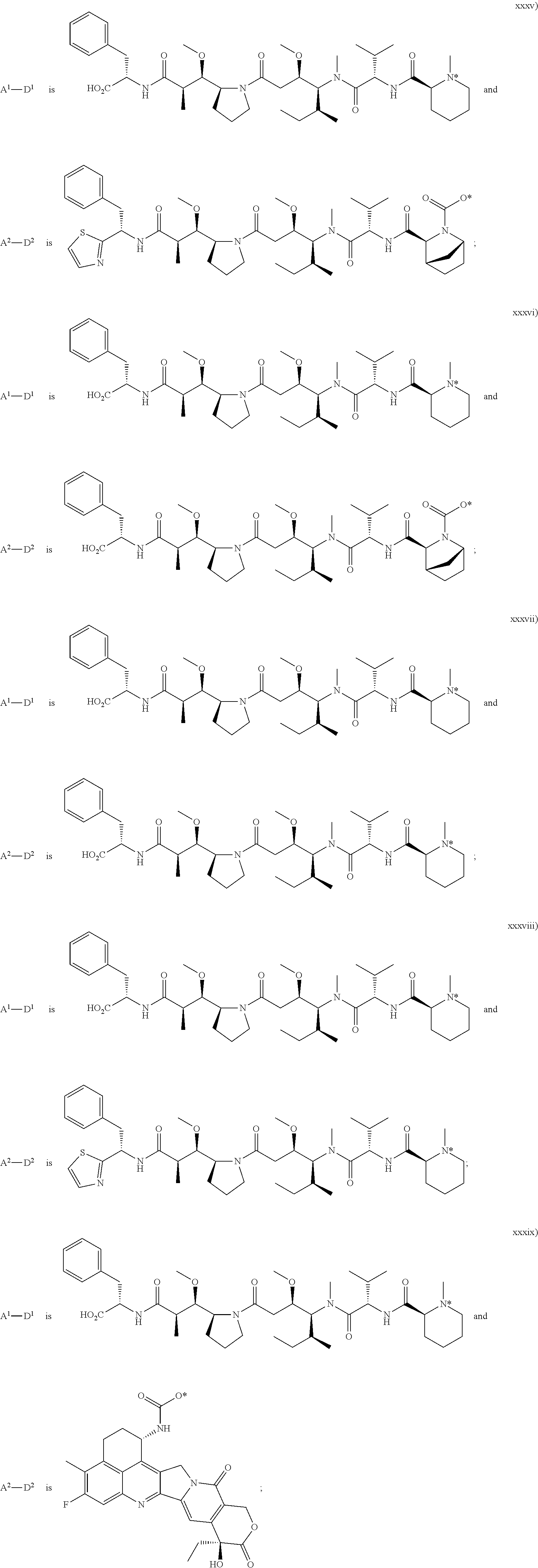

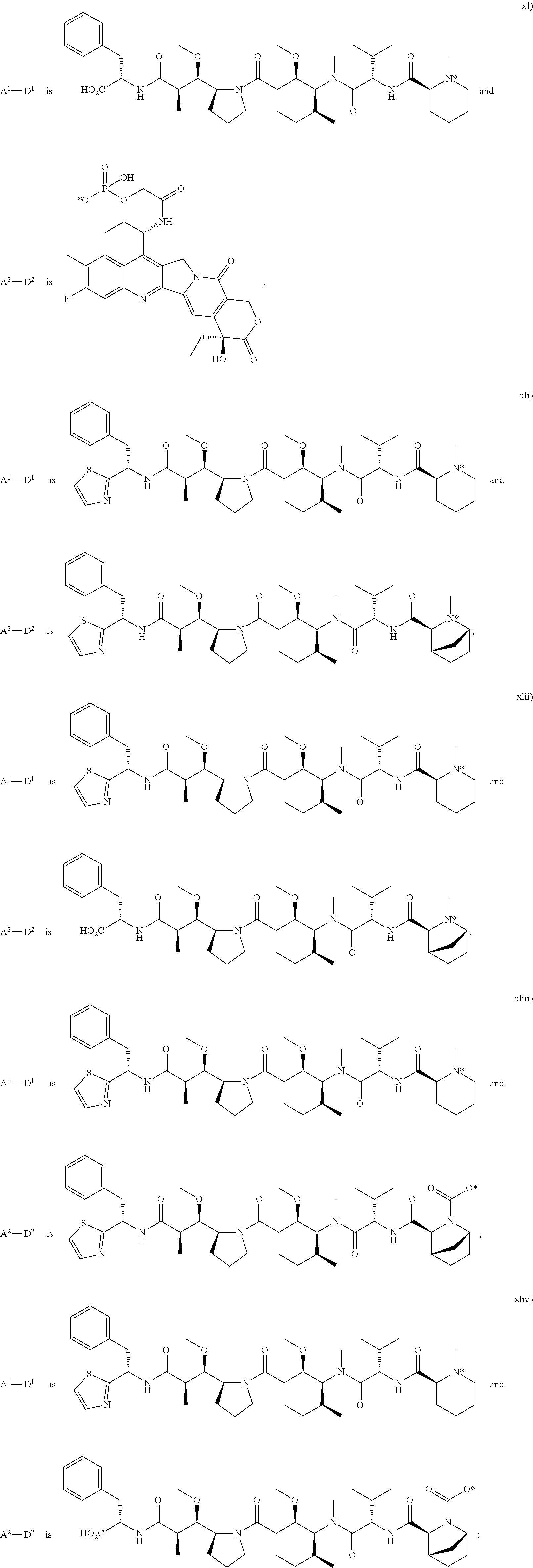

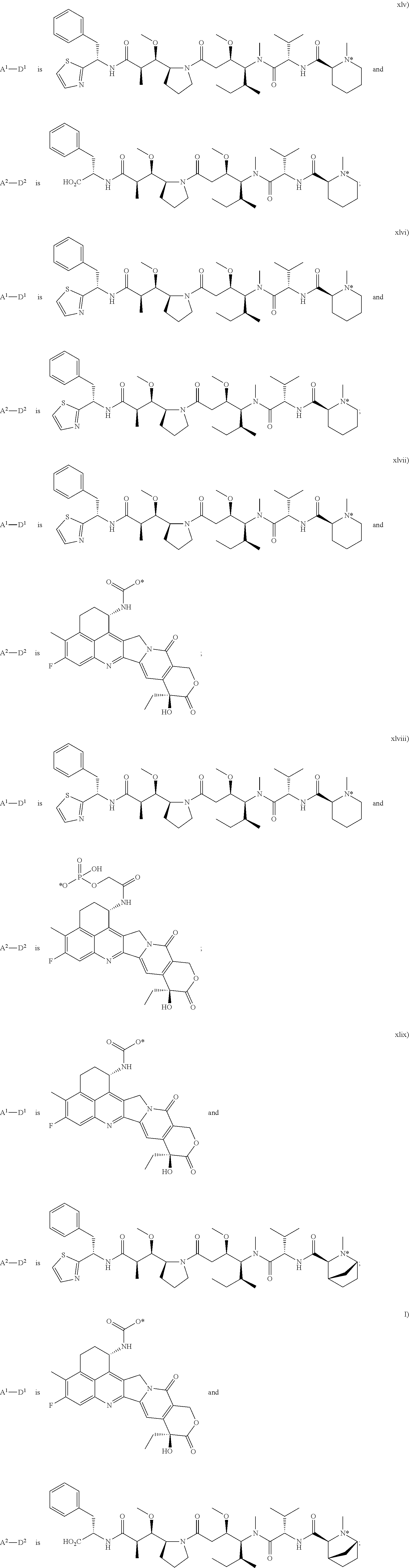

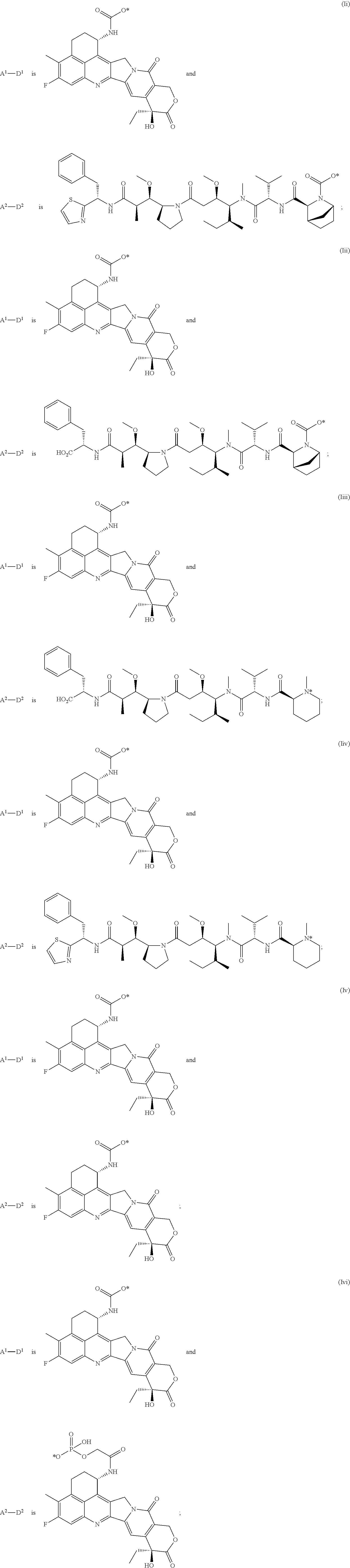

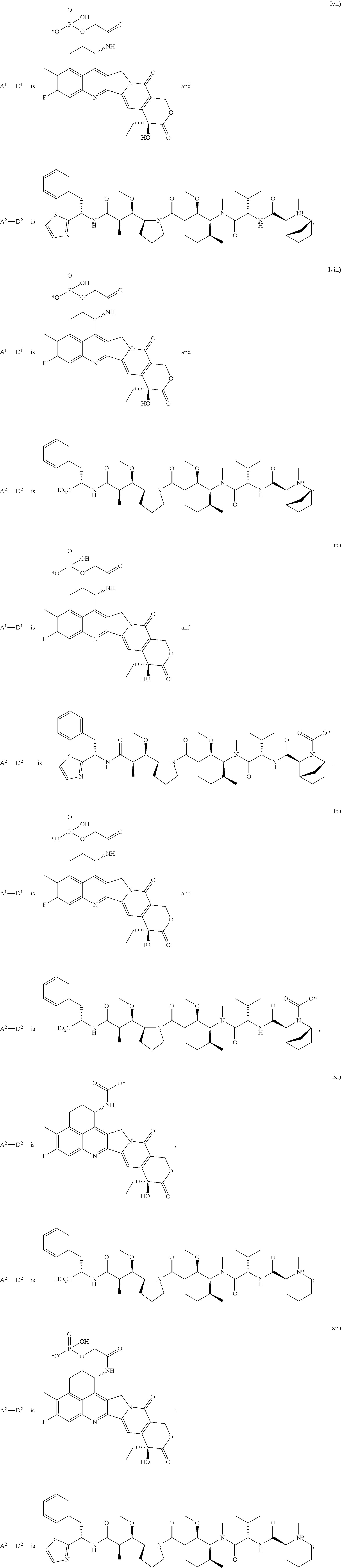

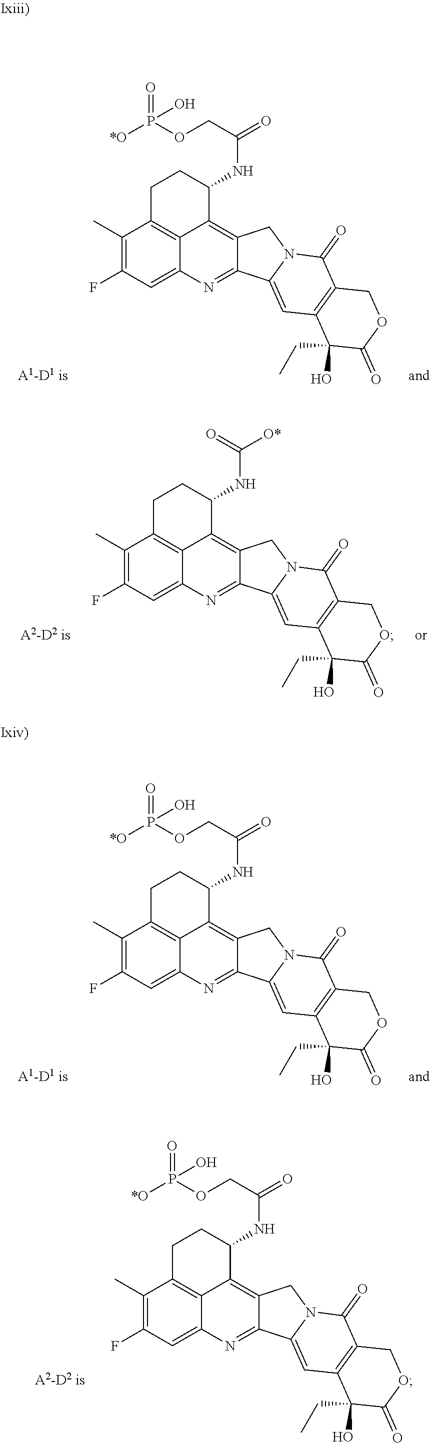

wherein * indicates the point of connection to the remainder of the molecule.

In an Embodiment, D¹ and/or D² are an auristatin.

In an embodiment, both D¹ and D² are an auristatin.

In an embodiment, D¹ and/or D² are a topoisomerase 1 inhibitor.

In an embodiment, both D¹ and D² are a topoisomerase 1 inhibitor.

In an embodiment, one of D¹ and D² is an auristatin and the other of D¹ and D² is a topoisomerase 1 inhibitor.

In an embodiment, D¹ is an auristatin and D² is a topoisomerase 1 inhibitor.

In an embodiment, D¹ is a topoisomerase 1 inhibitor and D² is an auristatin.

In an Embodiment, D¹ and D² are independently selected from the group consisting of

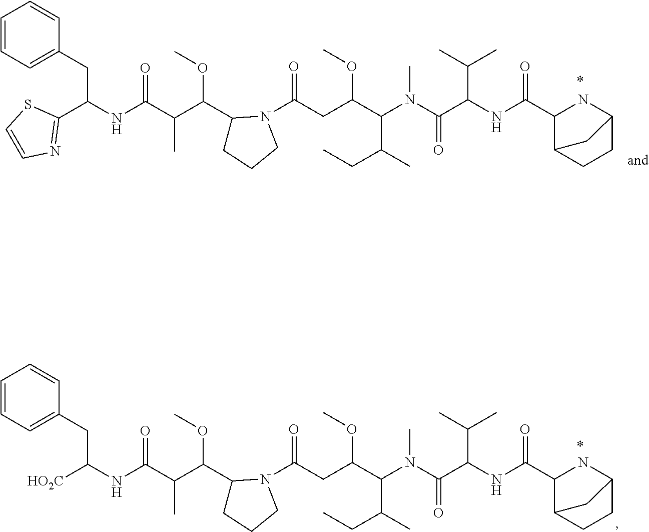

wherein * indicates the point of attachment to A¹ or A².

In an Embodiment, D¹ and D² are independently selected from the group consisting of

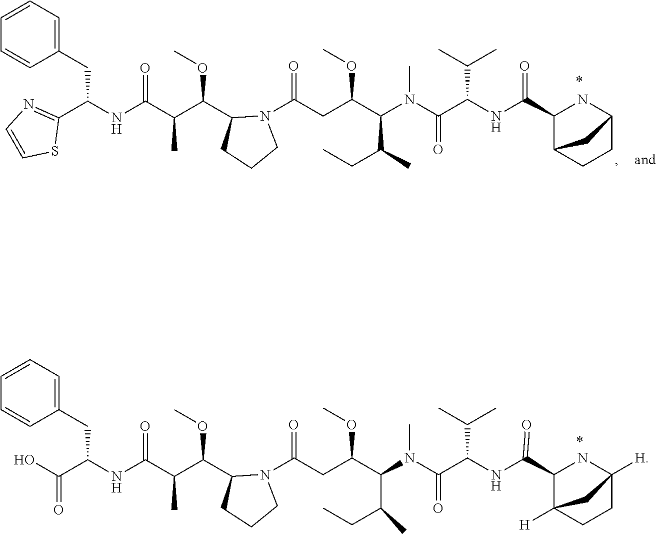

In an Embodiment, D¹ and D² are each independently selected from an antitubulin agent, a tubulin inhibitor, a DNA minor groove binder, a DNA replication inhibitor, an alkylating agent, an antibiotic, an antifolate, an antimetabolite, a chemotherapy sensitizer, a topoisomerase inhibitor, and/or a vinca alkaloid.

In an Embodiment, D¹ and D² are each independently selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor, a DHFR inhibitor, or a pro-apoptotic agent, optionally with the proviso that neither D¹ nor D² is a BH3 mimetic.

In an Embodiment, the antibody or antigen-binding fragment binds to a target antigen on a cancer cell.

In an Embodiment, (i) the target antigen is selected from BCMA, CD33, HER2, CD38, CD48, CD79b, PCAD, CD74, CD138, SLAMF7, CD123, CLL1, FLT3, CD7, CKIT, CD56, SEZ6, DLL3, DLK1, B7-H3, EGFR, CD71, EphA2, EPCAM, FOLR1, ENPP3, MET, AXL, SLC34A2, Nectin4, TROP2, LIV1, CD46, MSLN, F3, MUC16, SLC39A6, TFRC, TACSTD2, and GPNMB; (ii) the target antigen is selected from EGFR, CD7, HER2, EPCAM, FOLR1, ENPP3, MET, AXL, SLC34A2, Nectin4, MSLN, F3, MUC16, SLC39A6, TFRC, TACSTD2, and GPNMB; or (iii) the target antigen is PCAD, CD48, CD74, EphA2, HER2, TROP2, B7-H3, or 5T4.

In an Embodiment, the target antigen is PCAD, CD48, CD74, EphA2, HER2, TROP2, B7-H3, or 5T4.

In an Embodiment, the antibody or antigen-binding fragment thereof is selected from Table D1.

In an Embodiment, the antibody or antigen-binding fragment comprises i) three heavy chain CDR sequences and three light chain CDR sequences selected from an antibody in Tables D3 and D8, ii) a heavy chain variable region sequence and a light chain variable region sequence selected from an antibody in Tables D2 and D8, and/or iii) a heavy chain sequence and light chain sequence selected from an antibody in Tables D4, D5, and D7.

In an Embodiment, the antibody or antigen-binding fragment is an anti-CD74 antibody comprising three heavy chain CDRs and three light chain CDRs selected from the group consisting of:
1) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:268, light chain CDR2 (LCDR2) consisting of SEQ ID NO:264, and light chain CDR3 (LCDR3) consisting of SEQ ID NO:265;
2) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
3) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:269, light chain CDR2 (LCDR2) consisting of SEQ ID NO:264, and light chain CDR3 (LCDR3) consisting of SEQ ID NO:174;
4) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
5) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:263, light chain CDR2 (LCDR2) consisting of SEQ ID NO:264, and light chain CDR3 (LCDR3) consisting of SEQ ID NO:265;
6) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174;
7) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:215, light chain CDR2 (LCDR2) consisting of SEQ ID NO:264, and light chain CDR3 (LCDR3) consisting of SEQ ID NO:174; and
8) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174.

In an Embodiment, the antibody or antigen-binding fragment is an anti-CD74 antibody comprising (a) a heavy chain variable region comprising the amino acid sequence of SEQ ID NO:153, and a light chain variable region comprising the amino acid sequence of SEQ ID NO:262, or (b) a heavy chain variable region comprising the amino acid sequence of SEQ ID NO:153, and a light chain variable region comprising the amino acid sequence of SEQ ID NO:267.

In an Embodiment, the antibody or antigen-binding fragment is an anti-CD74 antibody comprising:
(a) the heavy chain amino acid sequence of SEQ ID NO: 118 or a sequence that is at least 95% identical to SEQ ID NO: 118, and the light chain amino acid sequence of SEQ ID NO:237 or a sequence that is at least 95% identical to SEQ ID NO:237;
(b) the heavy chain amino acid sequence of SEQ ID NO:236 or a sequence that is at least 95% identical to SEQ ID NO:236, and the light chain amino acid sequence of SEQ ID NO:237 or a sequence that is at least 95% identical to SEQ ID NO:237; or
(c) the heavy chain amino acid sequence of SEQ ID NO: 118 or a sequence that is at least 95% identical to SEQ ID NO:118, and the light chain amino acid sequence of SEQ ID NO:239 or a sequence that is at least 95% identical to SEQ ID NO:239.

In an Embodiment, the antibody or antigen-binding fragment is an anti-CD48 antibody comprising three heavy chain CDRs and three light chain CDRs selected from the group consisting of:
1) heavy chain CDR1 (HCDR1) consisting of SEQ ID NO:271, heavy chain CDR2 (HCDR2) consisting of SEQ ID NO:272, heavy chain CDR3 (HCDR3) consisting of SEQ ID NO:273; light chain CDR1 (LCDR1) consisting of SEQ ID NO:281, light chain CDR2 (LCDR2) consisting of SEQ ID NO:282, and light chain CDR3 (LCDR3) consisting of SEQ ID NO:283;
2) heavy chain CDR1 (HCDR1) consisting of SEQ ID NO:274, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:285, and light chain CDR3 (LCDR3) consisting of SEQ ID NO:286;
3) heavy chain CDR1 (HCDR1) consisting of SEQ ID NO:276, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:287, light chain CDR2 (LCDR2) consisting of SEQ ID NO:282, and light chain CDR3 (LCDR3) consisting of SEQ ID NO:286; and
4) heavy chain CDR1 (HCDR1) consisting of SEQ ID NO:279, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:288, and light chain CDR3 (LCDR3) consisting of SEQ ID NO:286; and
5) heavy chain CDR1 (HCDR1) consisting of SEQ ID NO:51, heavy chain CDR2 (HCDR2) consisting of SEQ ID NO:52, heavy chain CDR3 (HCDR3) consisting of SEQ ID NO:53; light chain CDR1 (LCDR1) consisting of SEQ ID NO:54, light chain CDR2 (LCDR2) consisting of SEQ ID NO:55, and light chain CDR3 (LCDR3) consisting of SEQ ID NO:56.

In an Embodiment, the antibody or antigen-binding fragment is an anti-CD48 antibody comprising a) a heavy chain variable region comprising the amino acid sequence of SEQ ID NO:270, and a light chain variable region comprising the amino acid sequence of SEQ ID NO:280; or b) a heavy chain variable region comprising the amino acid sequence of SEQ ID NO:13, and a light chain variable region comprising the amino acid sequence of SEQ ID NO:14.

In an Embodiment, the antibody or antigen-binding fragment is an anti-CD48 antibody comprising (a) the heavy chain amino acid sequence of SEQ ID NO:240 or a sequence that is at least 95% identical to SEQ ID NO:240, and the light chain amino acid sequence of SEQ ID NO:243 or a sequence that is at least 95% identical to SEQ ID NO:243; or (b) the heavy chain amino acid sequence of SEQ ID NO:242 or a sequence that is at least 95% identical to SEQ ID NO:242, and the light chain amino acid sequence of SEQ ID NO:243 or a sequence that is at least 95% identical to SEQ ID NO:243; c) the heavy chain amino acid sequence of SEQ ID NO:240 or a sequence that is at least 95% identical to SEQ ID NO:240, and the light chain amino acid sequence of SEQ ID NO:69 or a sequence that is at least 95% identical to SEQ ID NO:70.

In an embodiment, the antibody or antigen-binding fragment is an anti-Her2 antibody comprising three heavy chain CDRs and three light chain CDRs selected from the group consisting of:
1) heavy chain CDR1 (HCDR1) consisting of SEQ ID NO:289, heavy chain CDR2 (HCDR2) consisting of SEQ ID NO:290, heavy chain CDR3 (HCDR3) consisting of SEQ ID NO:291; light chain CDR1 (LCDR1) consisting of SEQ ID NO:297, light chain CDR2 (LCDR2) consisting of SEQ ID NO:298, and light chain CDR3 (LCDR3) consisting of SEQ ID NO:299;
2) heavy chain CDR1 (HCDR1) consisting of SEQ ID NO:292, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44;
3) heavy chain CDR1 (HCDR1) consisting of SEQ ID NO:293, heavy chain CDR2 (HCDR2) consisting of SEQ ID NO:294, heavy chain CDR3 (HCDR3) consisting of SEQ ID NO:295; light chain CDR1 (LCDR1) consisting of SEQ ID NO:302, light chain CDR2 (LCDR2) consisting of SEQ ID NO:298, and light chain CDR3 (LCDR3) consisting of SEQ ID NO:44; and
4) heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44.

In an embodiment, the antibody or antigen-binding fragment is an anti-Her2 antibody comprising a heavy chain variable region comprising the amino acid sequence of SEQ ID NO:9, and a light chain variable region comprising the amino acid sequence of SEQ ID NO:296.

In an embodiment, the antibody or antigen-binding fragment is an anti-Her2 antibody comprising the heavy chain amino acid sequence of SEQ ID NO:245 or a sequence that is at least 95% identical to SEQ ID NO:245, and the light chain amino acid sequence of SEQ ID NO:66 or a sequence that is at least 95% identical to SEQ ID NO:66.

In an embodiment, the antibody or antigen-binding fragment is an anti-PCAD antibody comprising three heavy chain CDRs and three light chain CDRs selected from the group consisting of:
1) heavy chain CDR1 (HCDR1) consisting of SEQ ID NO:304, heavy chain CDR2 (HCDR2) consisting of SEQ ID NO:305, heavy chain CDR3 (HCDR3) consisting of SEQ ID NO:306; light chain CDR1 (LCDR1) consisting of SEQ ID NO:312, light chain CDR2 (LCDR2) consisting of SEQ ID NO:313, and light chain CDR3 (LCDR3) consisting of SEQ ID NO:314;
2) heavy chain CDR1 (HCDR1) consisting of SEQ ID NO:307, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316;
3) heavy chain CDR1 (HCDR1) consisting of SEQ ID NO:309, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:317, light chain CDR2 (LCDR2) consisting of SEQ ID NO:313, and light chain CDR3 (LCDR3) consisting of SEQ ID NO:316; and
4) heavy chain CDR1 (HCDR1) consisting of SEQ ID NO:310, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316.

In an embodiment, the antibody or antigen-binding fragment is an anti-PCAD antibody comprising a heavy chain variable region comprising the amino acid sequence of SEQ ID NO:303, and a light chain variable region comprising the amino acid sequence of SEQ ID NO:311.

In an embodiment, the antibody or antigen-binding fragment is an anti-PCAD antibody comprising the heavy chain amino acid sequence of SEQ ID NO:248 or a sequence that is at least 95% identical to SEQ ID NO:248, and the light chain amino acid sequence of SEQ ID NO:250 or a sequence that is at least 95% identical to SEQ ID NO:250.

In an embodiment, the antibody or antigen-binding fragment is an anti-EphA2 antibody comprising three heavy chain CDRs and three light chain CDRs selected from the group consisting of:
1) heavy chain CDR1 (HCDR1) consisting of SEQ ID NO:319, heavy chain CDR2 (HCDR2) consisting of SEQ ID NO:320, heavy chain CDR3 (HCDR3) consisting of SEQ ID NO:321; light chain CDR1 (LCDR1) consisting of SEQ ID NO:330, light chain CDR2 (LCDR2) consisting of SEQ ID NO:331, and light chain CDR3 (LCDR3) consisting of SEQ ID NO:332;
2) heavy chain CDR1 (HCDR1) consisting of SEQ ID NO:322, heavy chain CDR2 (HCDR2) consisting of SEQ ID NO:323, heavy chain CDR3 (HCDR3) consisting of SEQ ID NO:324; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335;
3) heavy chain CDR1 (HCDR1) consisting of SEQ ID NO:325, heavy chain CDR2 (HCDR2) consisting of SEQ ID NO:326, heavy chain CDR3 (HCDR3) consisting of SEQ ID NO:327; light chain CDR1 (LCDR1) consisting of SEQ ID NO:336, light chain CDR2 (LCDR2) consisting of SEQ ID NO:331, and light chain CDR3 (LCDR3) consisting of SEQ ID NO:335; and
4) heavy chain CDR1 (HCDR1) consisting of SEQ ID NO:328, heavy chain CDR2 (HCDR2) consisting of SEQ ID NO:323, heavy chain CDR3 (HCDR3) consisting of SEQ ID NO:321; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.

In an embodiment, the antibody or antigen-binding fragment is an anti-EphA2 antibody comprising a heavy chain variable region comprising the amino acid sequence of SEQ ID NO:318, and a light chain variable region comprising the amino acid sequence of SEQ ID NO:329.

In an embodiment, the antibody or antigen-binding fragment is an anti-EphA2 antibody comprising the heavy chain amino acid sequence of SEQ ID NO:252 or a sequence that is at least 95% identical to SEQ ID NO:252, and the light chain amino acid sequence of SEQ ID NO:254 or a sequence that is at least 95% identical to SEQ ID NO:254.

In an embodiment, the antibody or antigen binding fragment thereof comprises one or more cysteine substitutions selected from E152C, S375C, or both E152C and S375C of the heavy chain of the antibody or antigen binding fragment thereof, wherein the position is numbered according to the EU system.

In an embodiment, the antibody or antigen binding fragment thereof comprises one or more Fc silencing mutations.

In an embodiment, the composition comprises multiple copies of the antibody-drug conjugate of any one of claims 46 to 110, wherein the average a of the antibody-drug conjugates in the composition is from about 1 to about 8, e.g., about 1 to about 6, about 1 to about 4, or about 1 to about 2.

In an embodiment, the compound or pharmaceutically acceptable salt there is represented by any one of the following formulae:

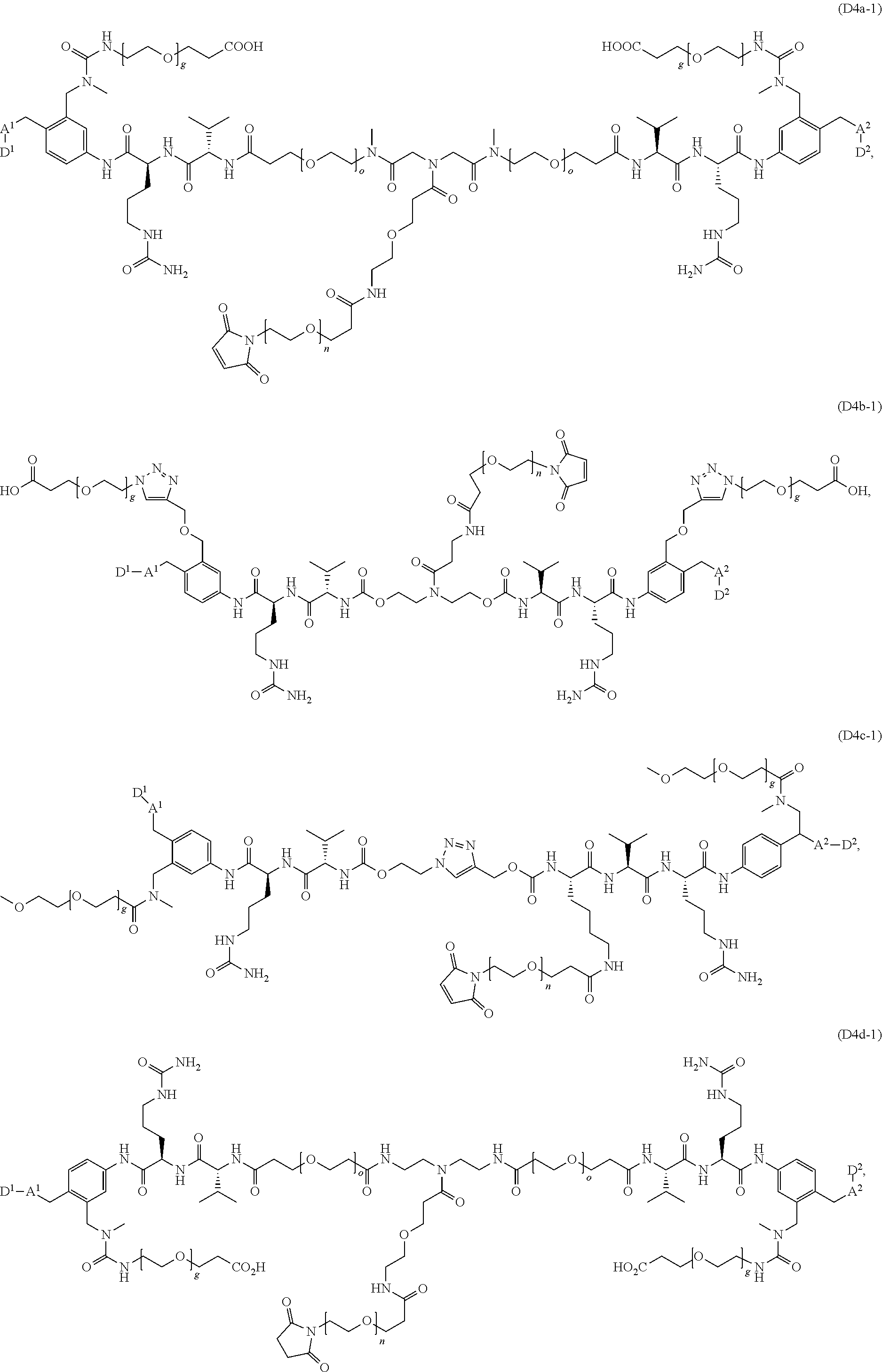

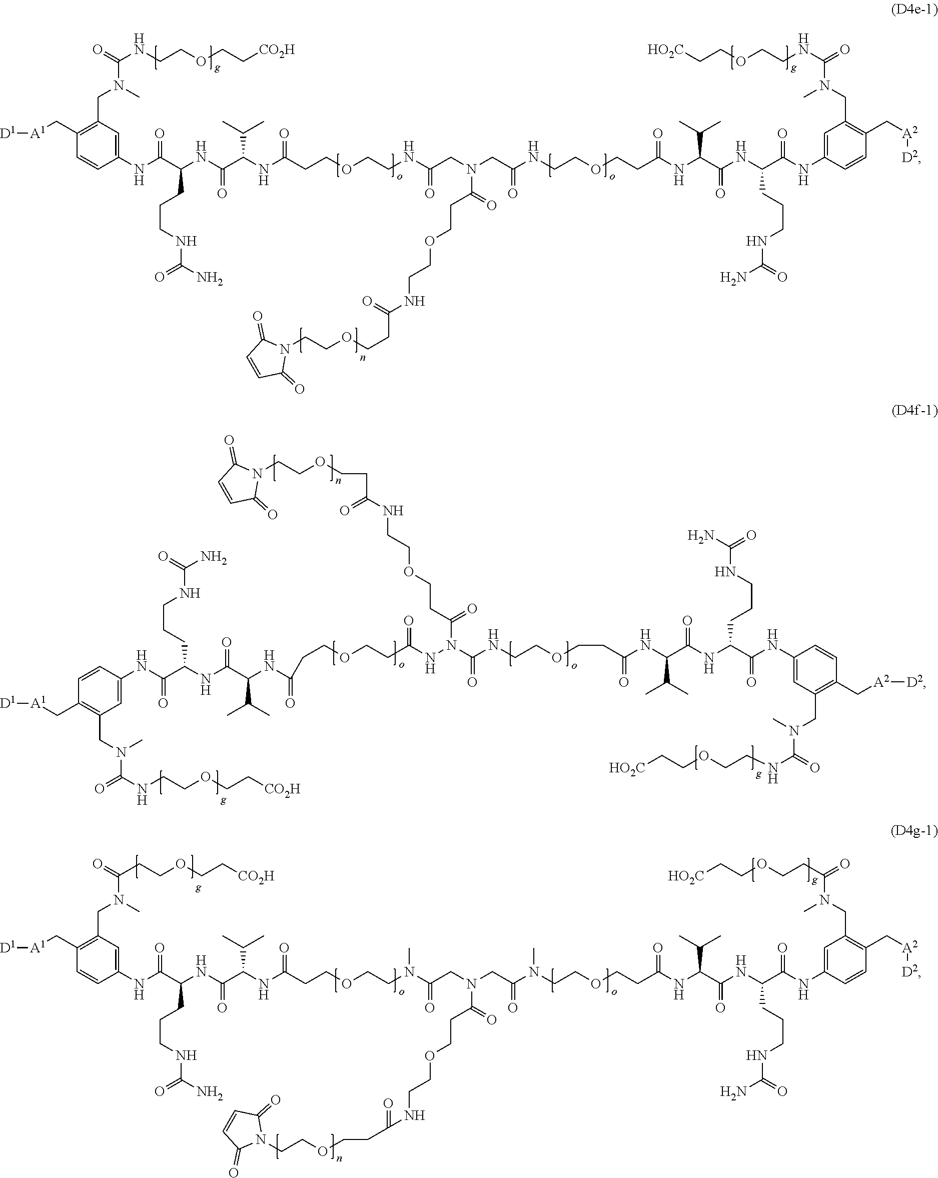

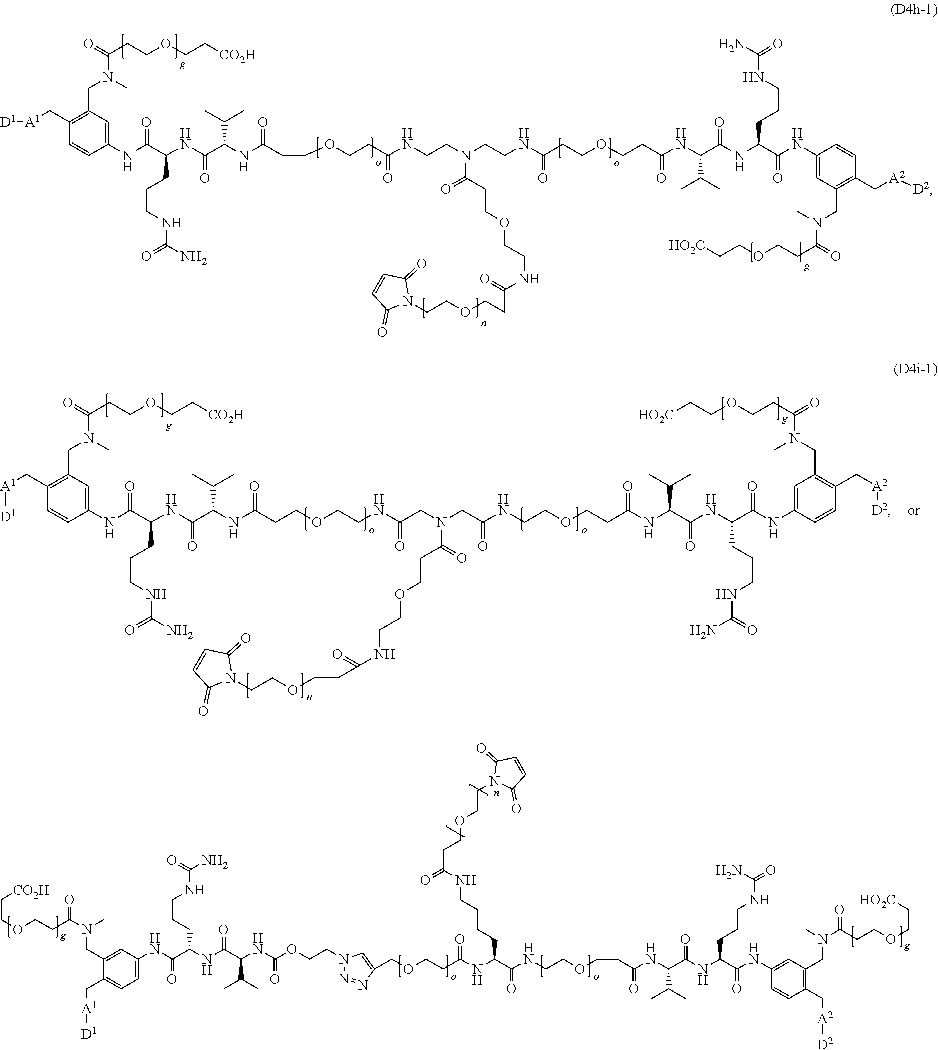

wherein:
A¹ and A² are each independently selected from a bond,

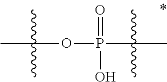 [SMILES: *OP(*)(=O)O]

and —O—C(═O)—*, wherein * in A¹ and A² indicates the point of attachment to D¹ or D²;
g for each occurrence is independently an integer between 20 and 30;
o for each occurrence is independently an integer between 1 and 9; and
n is an integer between 1 and 12.

In an embodiment, A¹ and A² are each independently selected from a bond and —O—C(═O)—*, wherein * in A¹ and A² indicates the point of attachment to D¹ or D².

In an embodiment, the conjugate is represented by any one of the following formulae:

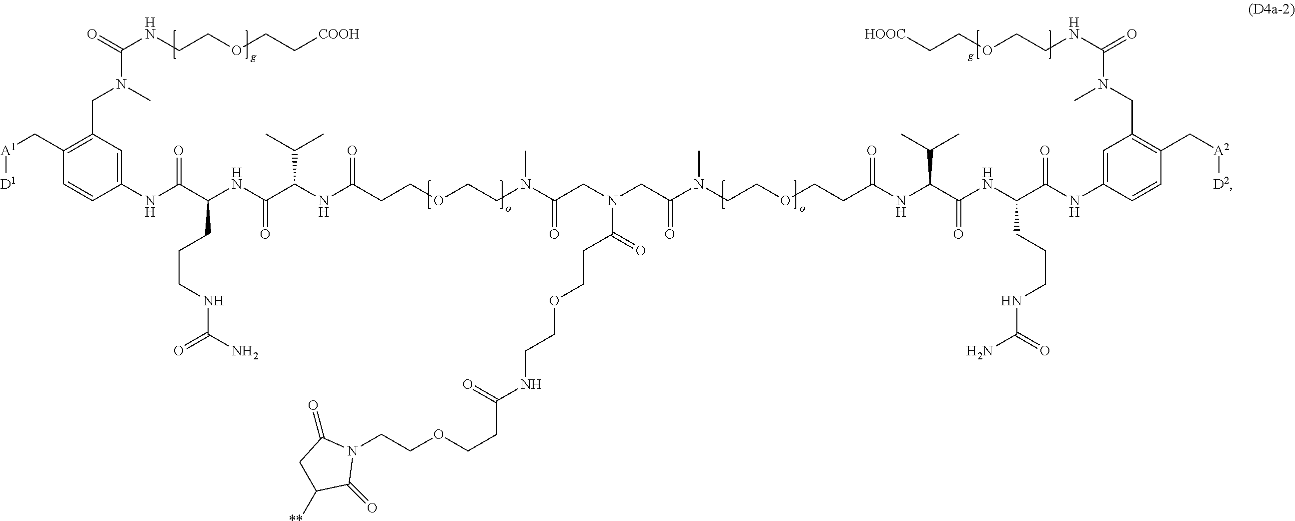

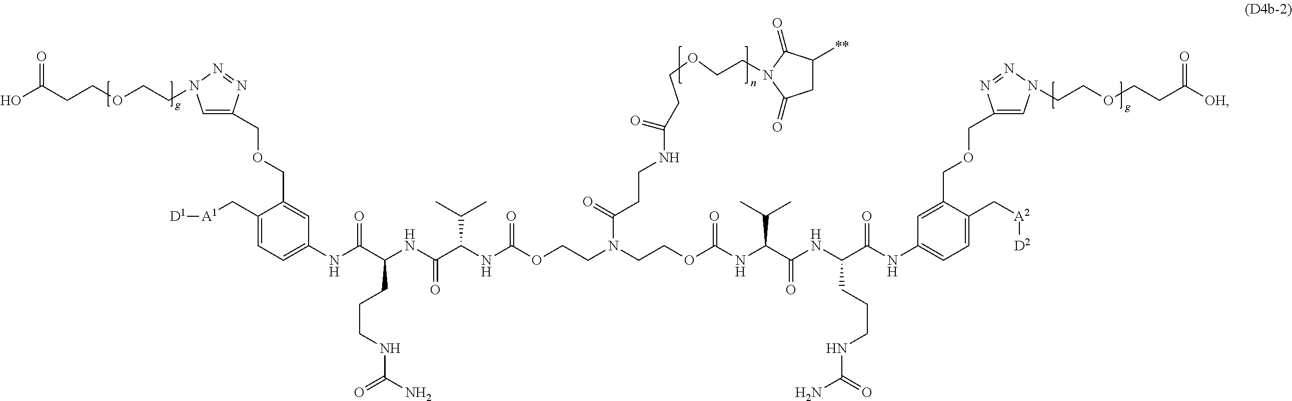

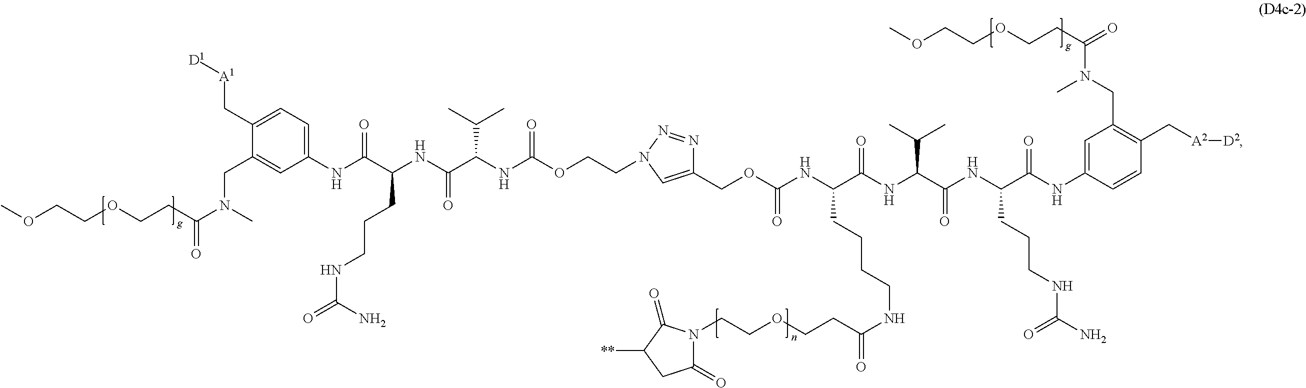

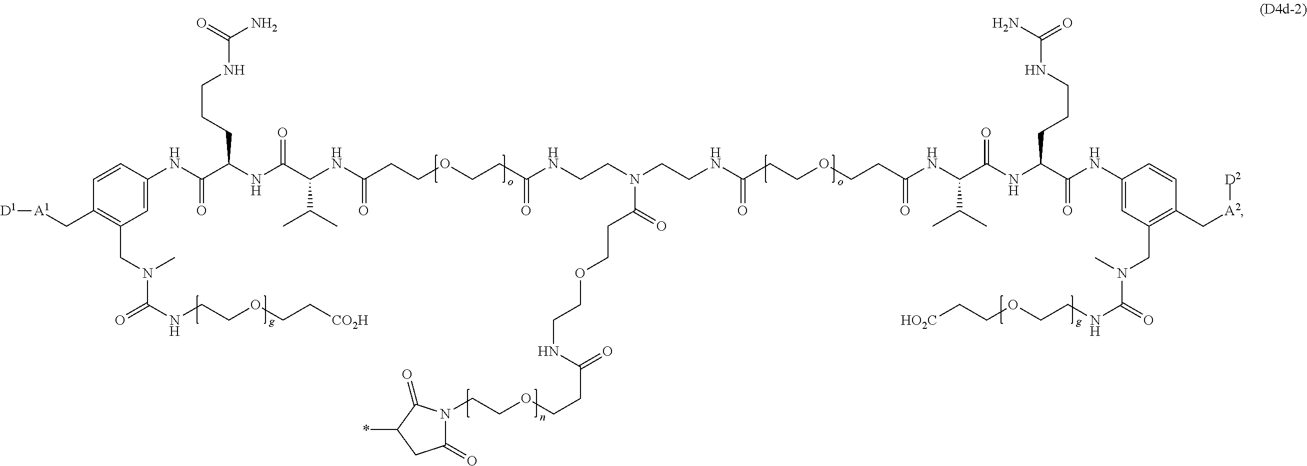

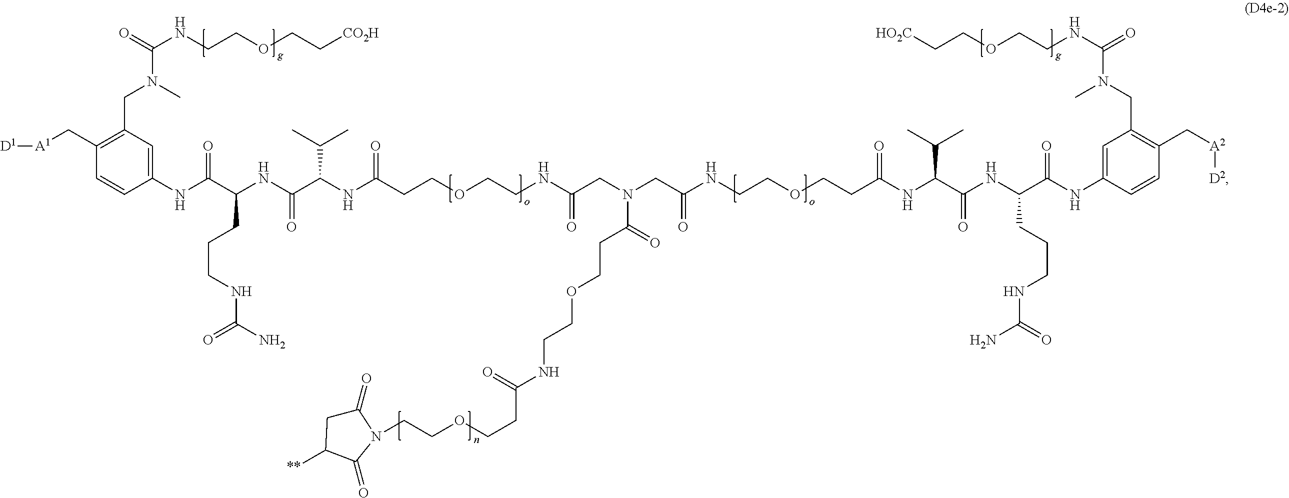

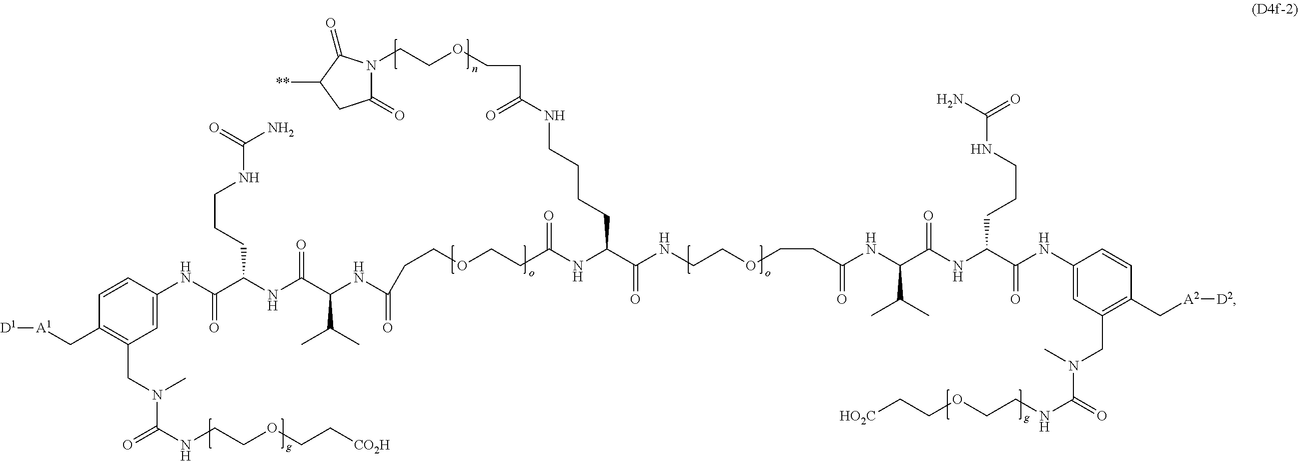

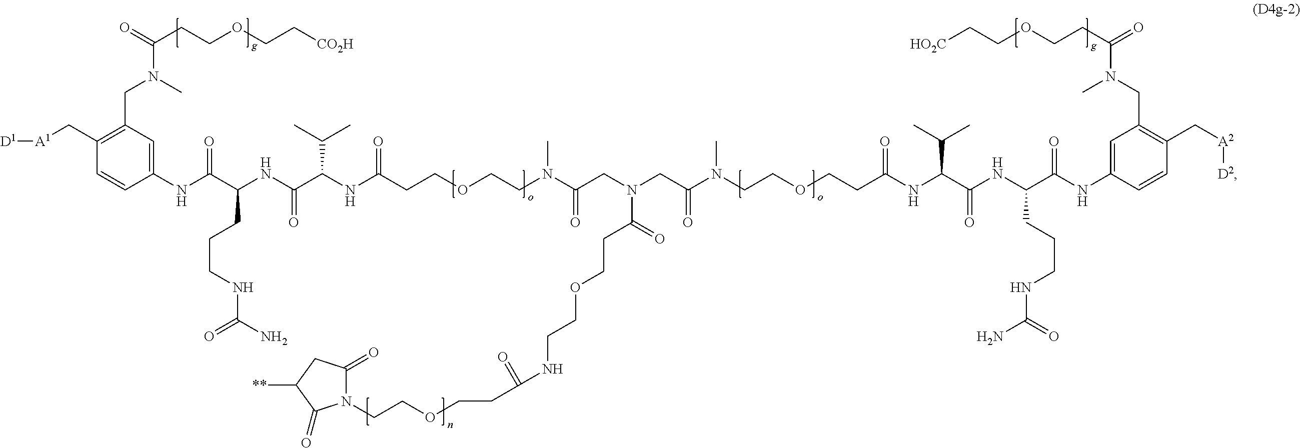

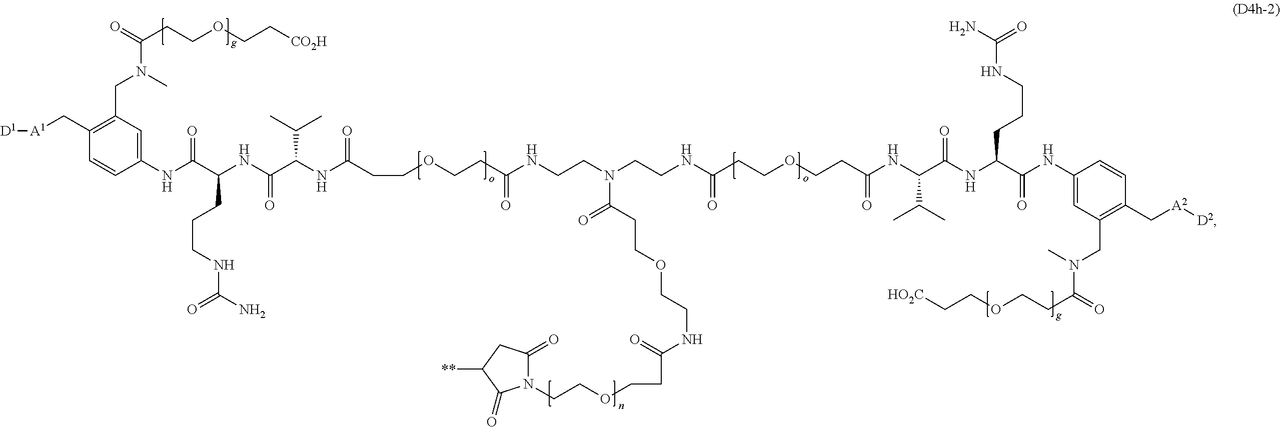

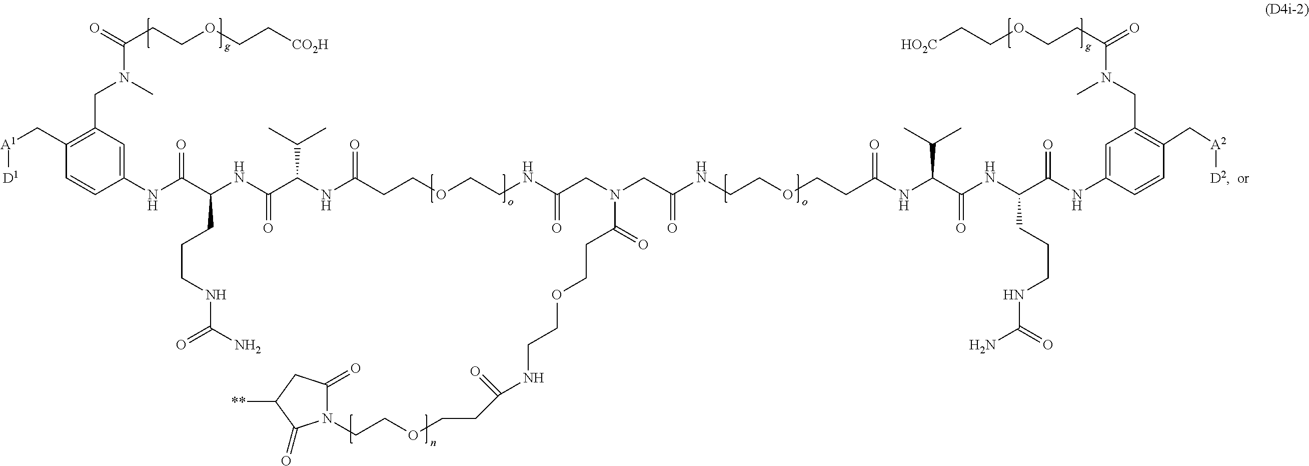

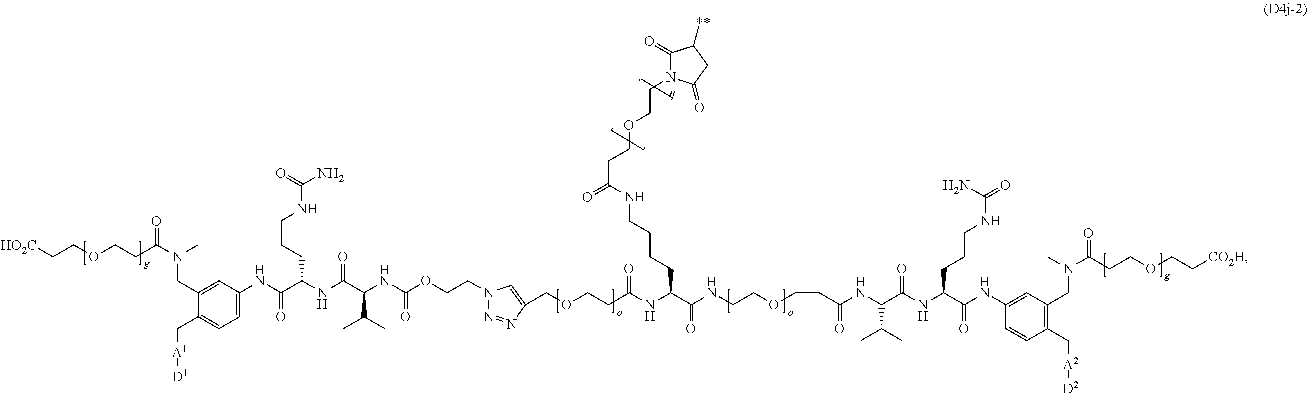

wherein:
A¹ and A² are each independently selected from a bond,

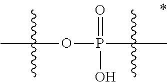 [SMILES: *OP(*)(=O)O]

and —O—C(═O)—*, wherein * in A¹ and A² indicates the point of attachment to D¹ or D².
g for each occurrence is independently an integer between 20 and 30;
o for each occurrence is independently an integer between 1 and 9;
n is an integer between 1 and 12; and [symbol] indicates the point of attachment to the Ab.
   In an embodiment, A¹ and A² are each independently selected from a bond and O—C(═O)—*, wherein * in A¹ and A² indicates the point of attachment to D¹ or D².

In an embodiment, the compound or pharmaceutically acceptable salt thereof is of formula (D5a-1):

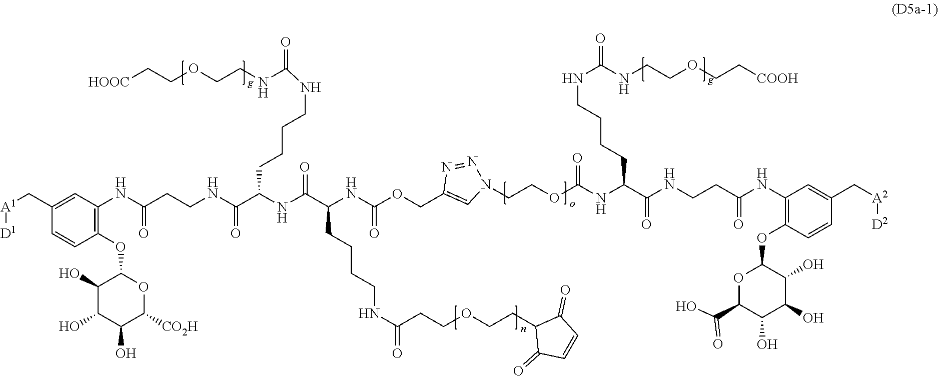

wherein:
A¹ and A² are each independently selected from a bond,

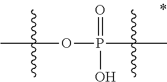 [SMILES: *OP(*)(=O)O]

and —O—C(═O)—*, wherein * in A¹ and A² indicates the point of attachment to D¹ or D².
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 1 and 3); and
n is an integer between 1 and 12 (e.g., between 5 and 10).
   In an Embodiment, A¹ and A² are each independently selected from a bond and —O—C(═O)—*, wherein * in A¹ and A² indicates the point of attachment to D¹ or D².

In an embodiment, the conjugate is of formula (D5a-2):

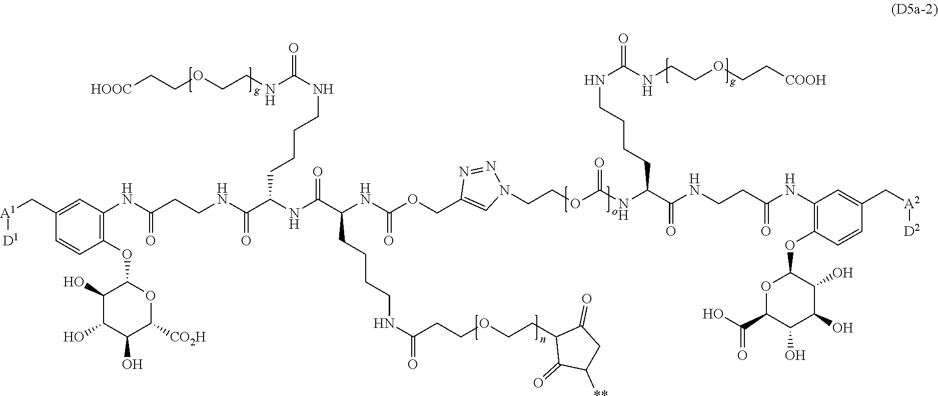

wherein:
A¹ and A² are each independently selected from a bond,

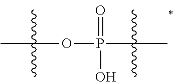 [SMILES: *OP(*)(=O)O]

and —O—C(═O)—*, wherein * in A¹ and A² indicates the point of attachment to D¹ or D².
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 1 and 3);
n is an integer between 1 and 12 (e.g., between 5 and 10); and
[symbol] indicates the point of attachment to the Ab.
   In an Embodiment, A¹ and A² are each independently selected from a bond and —O—C(═O)—*, wherein * in A¹ and A² indicates the point of attachment to D¹ or D².

In an Embodiment, the conjugate is represented by any one of the following formulae:

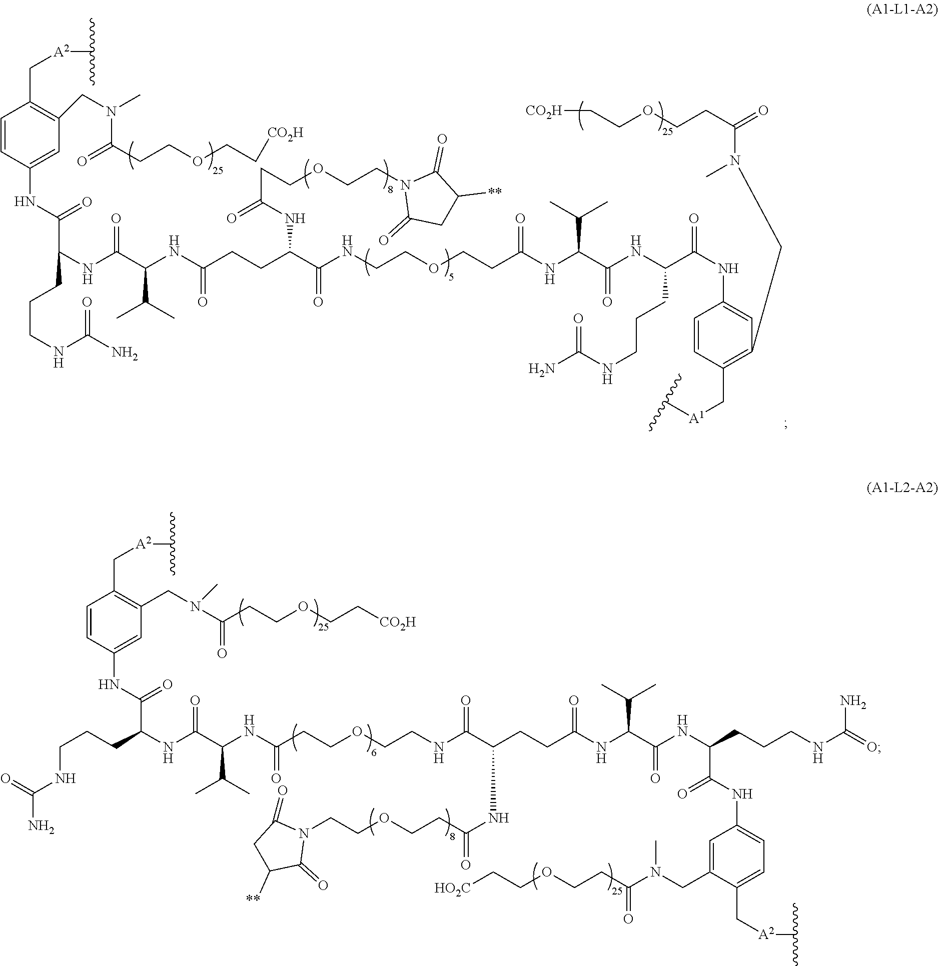

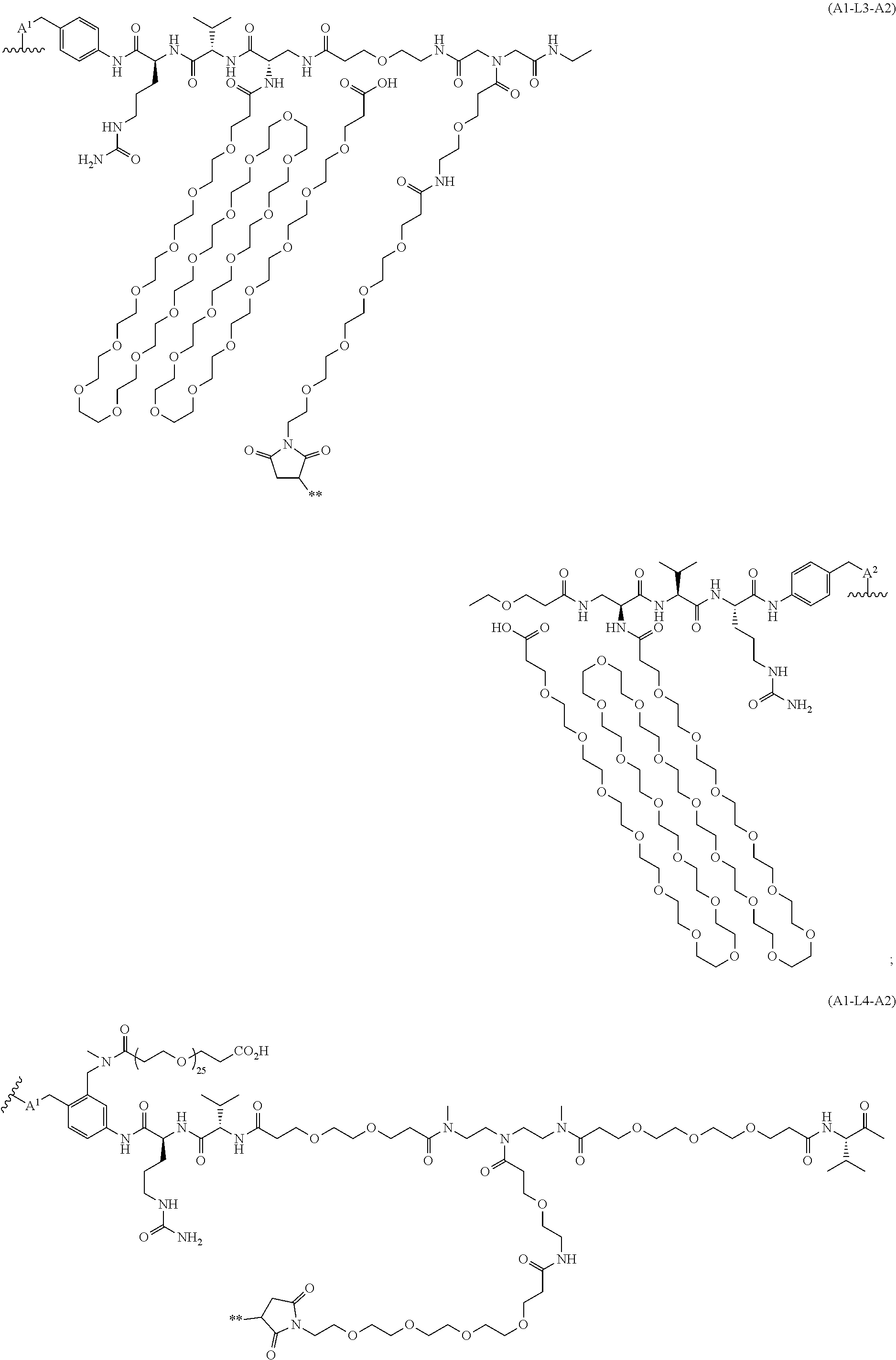

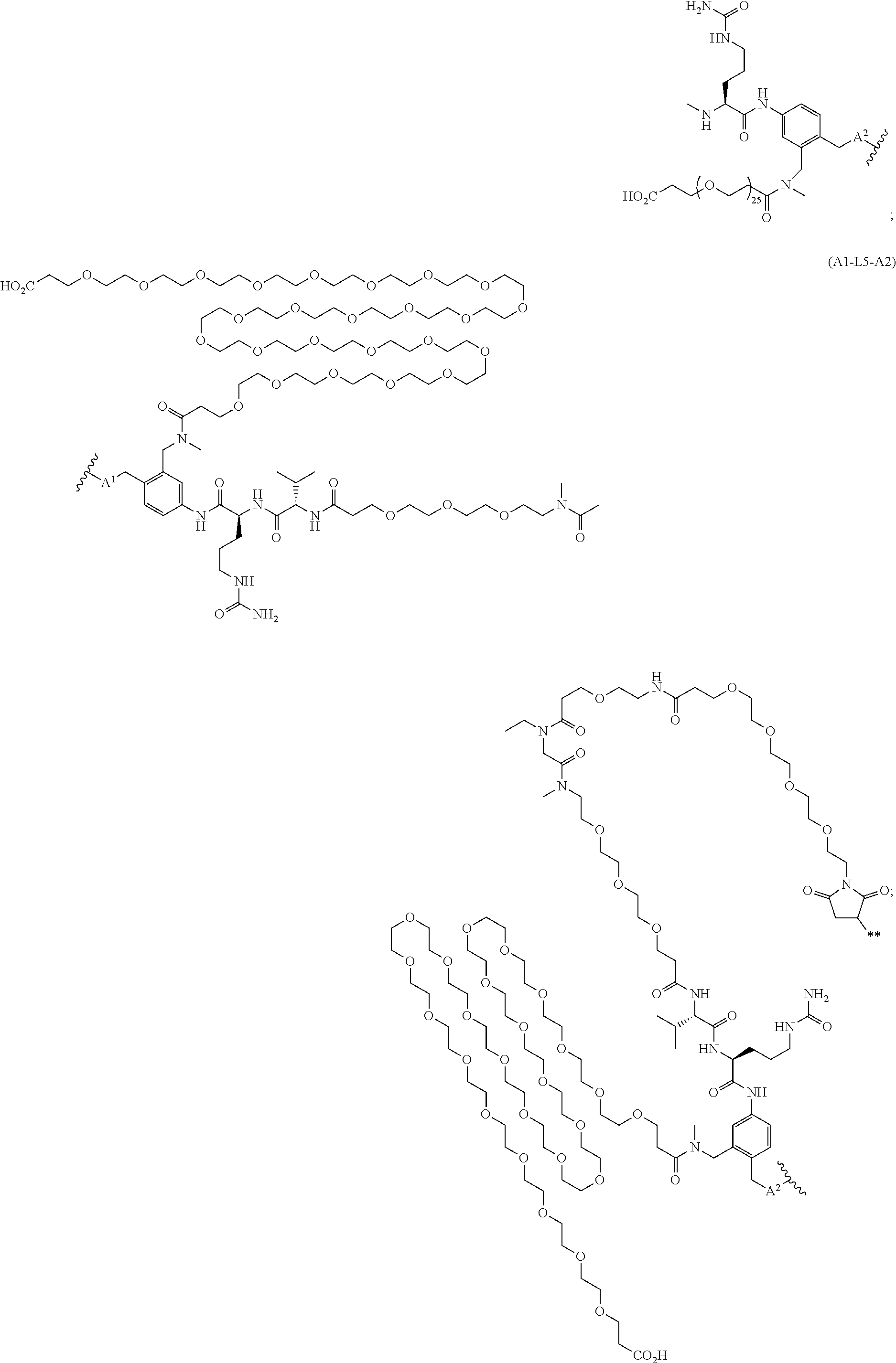

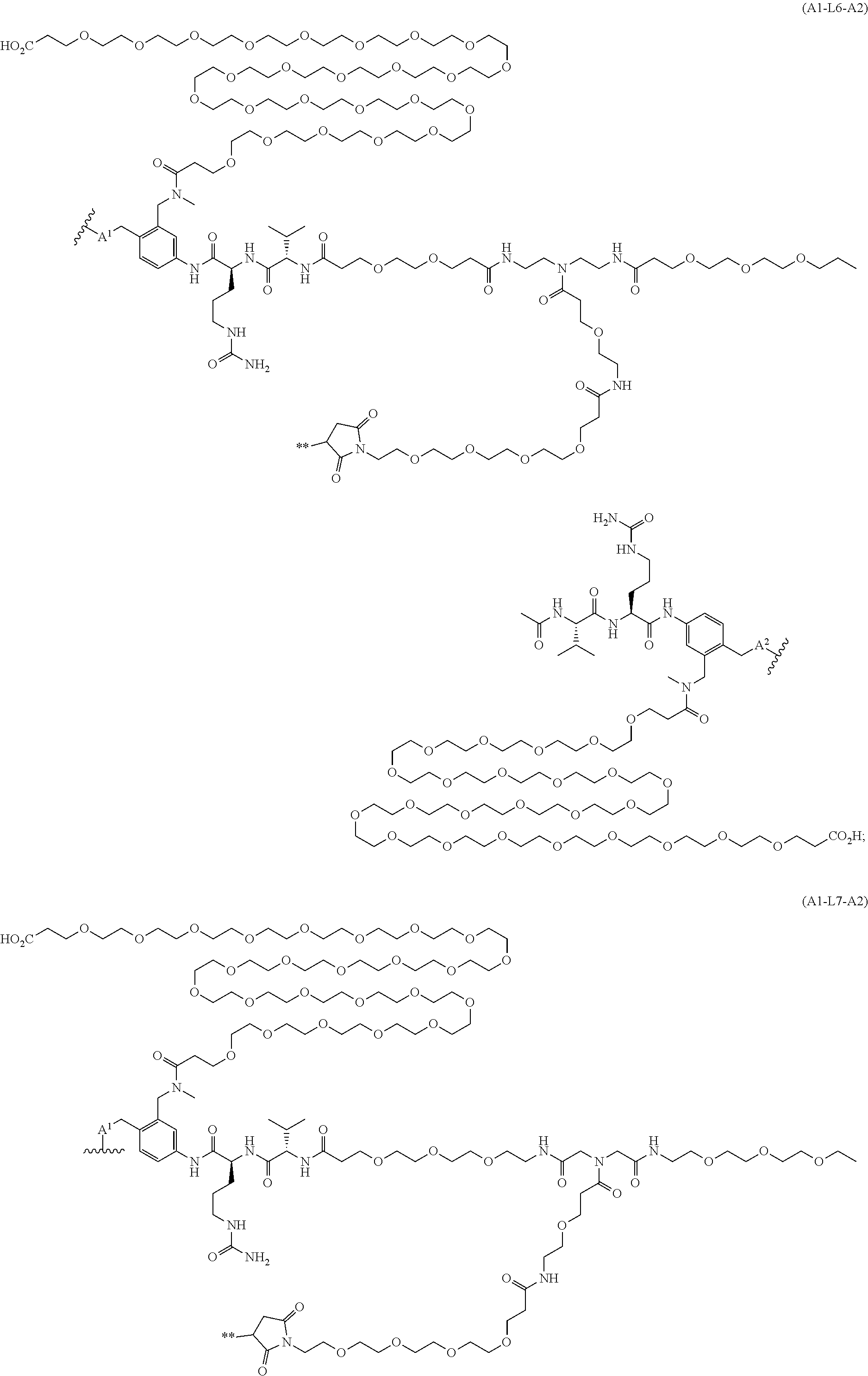

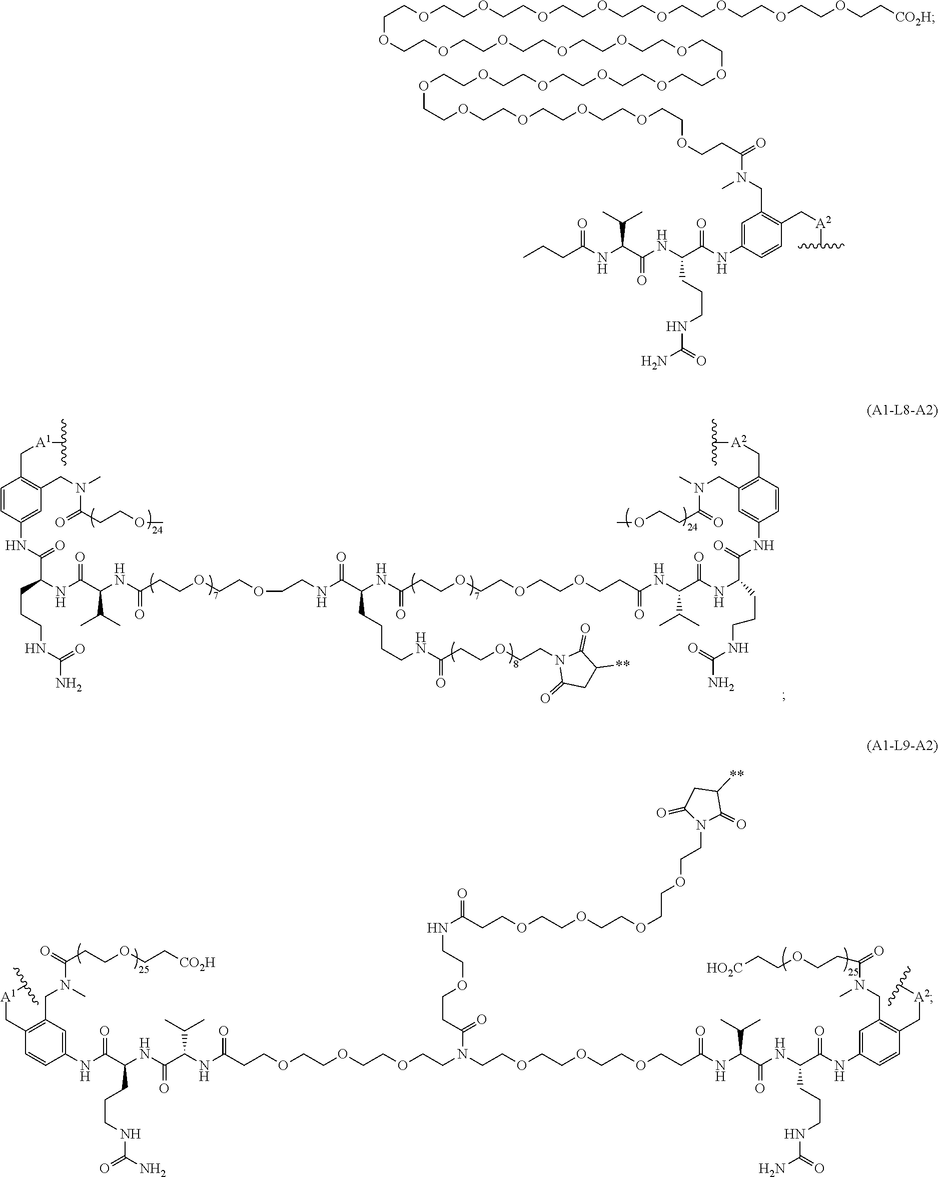

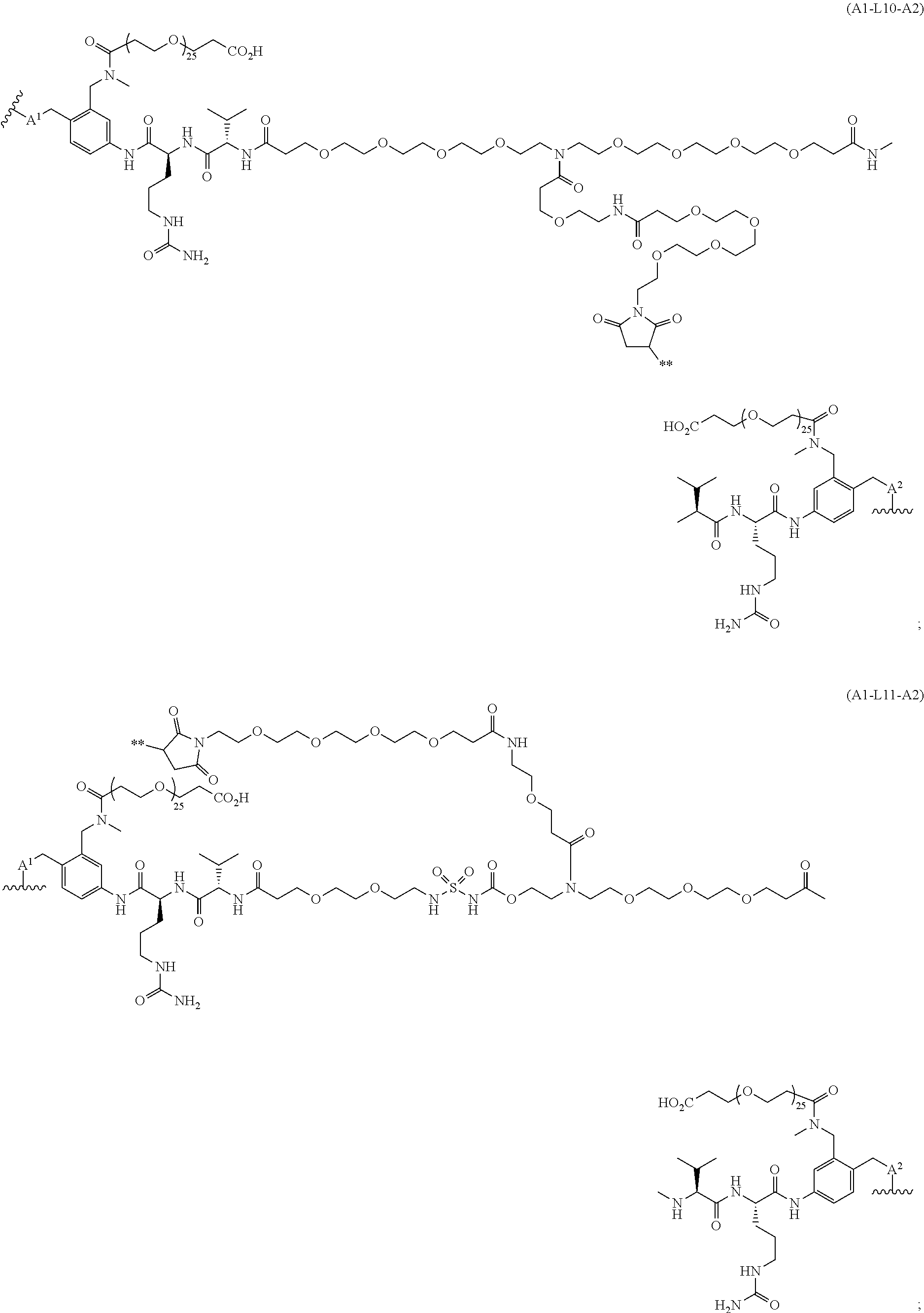

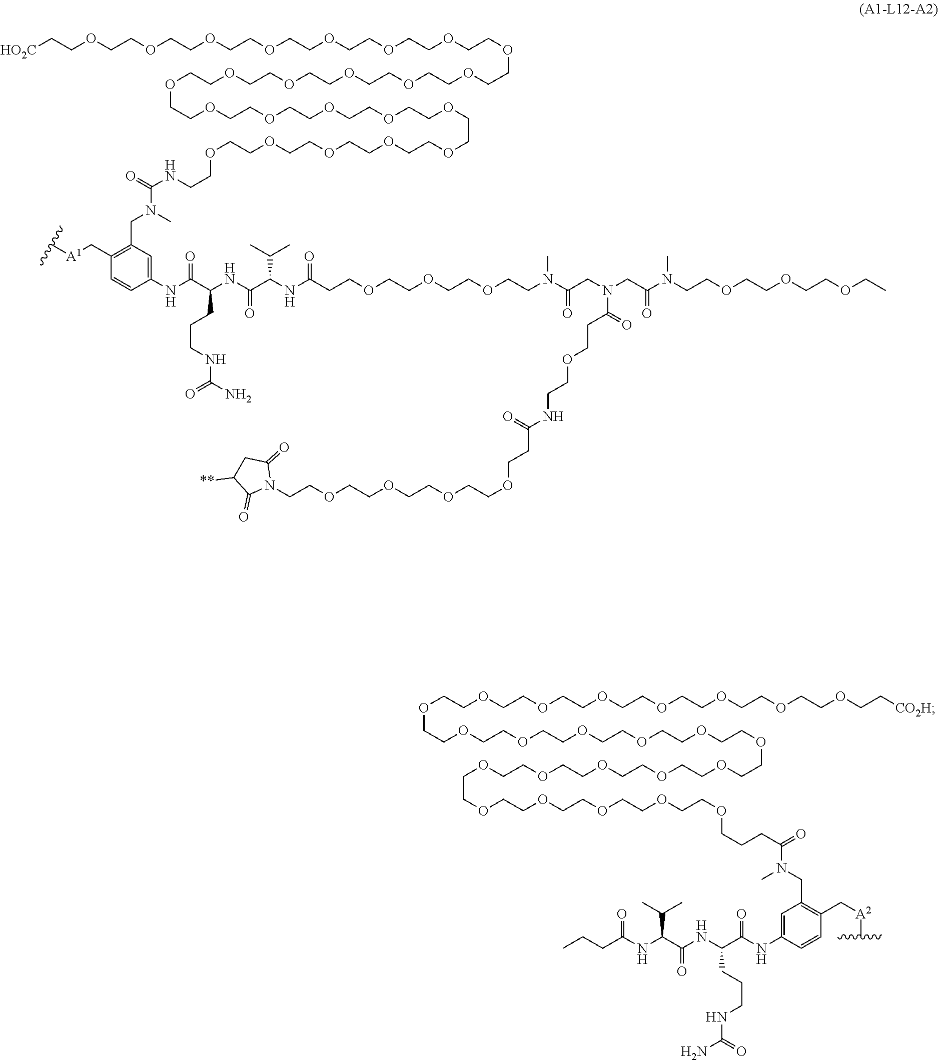

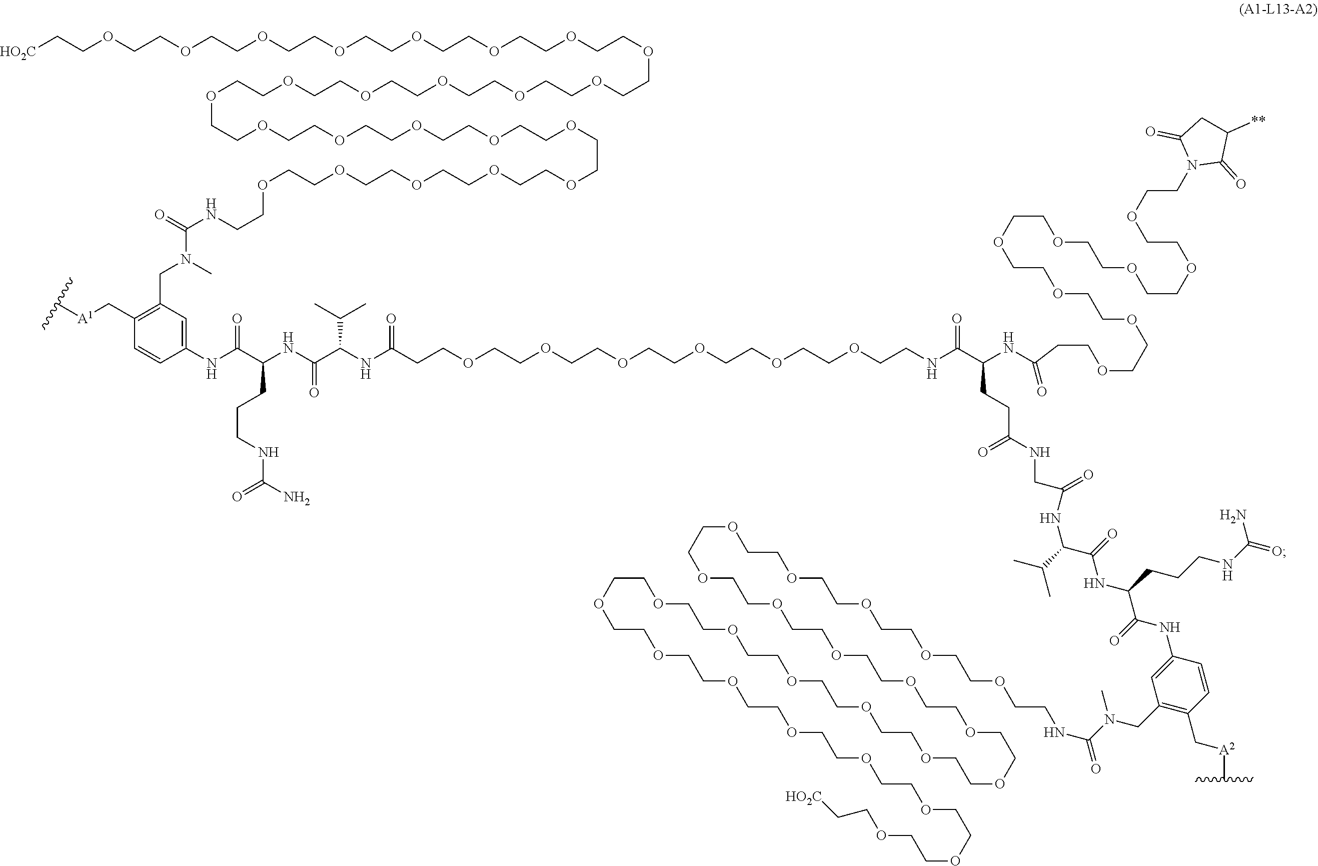

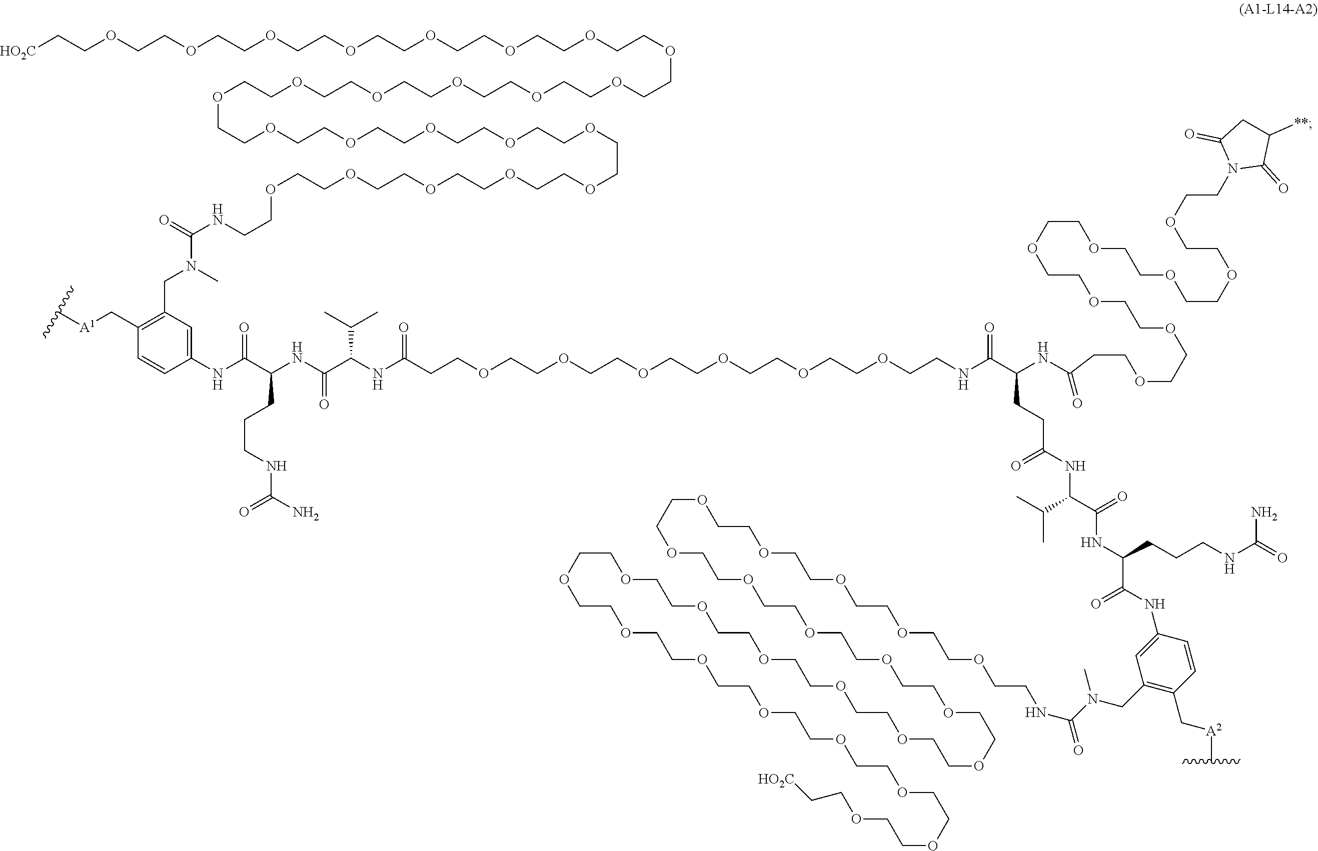

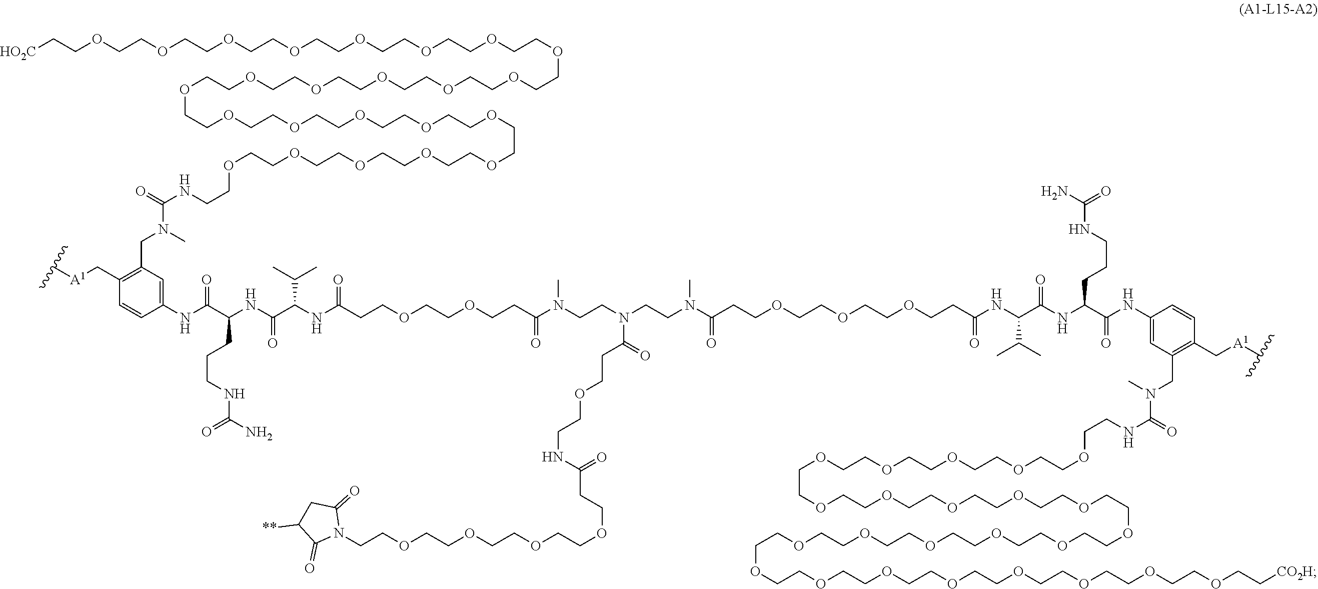

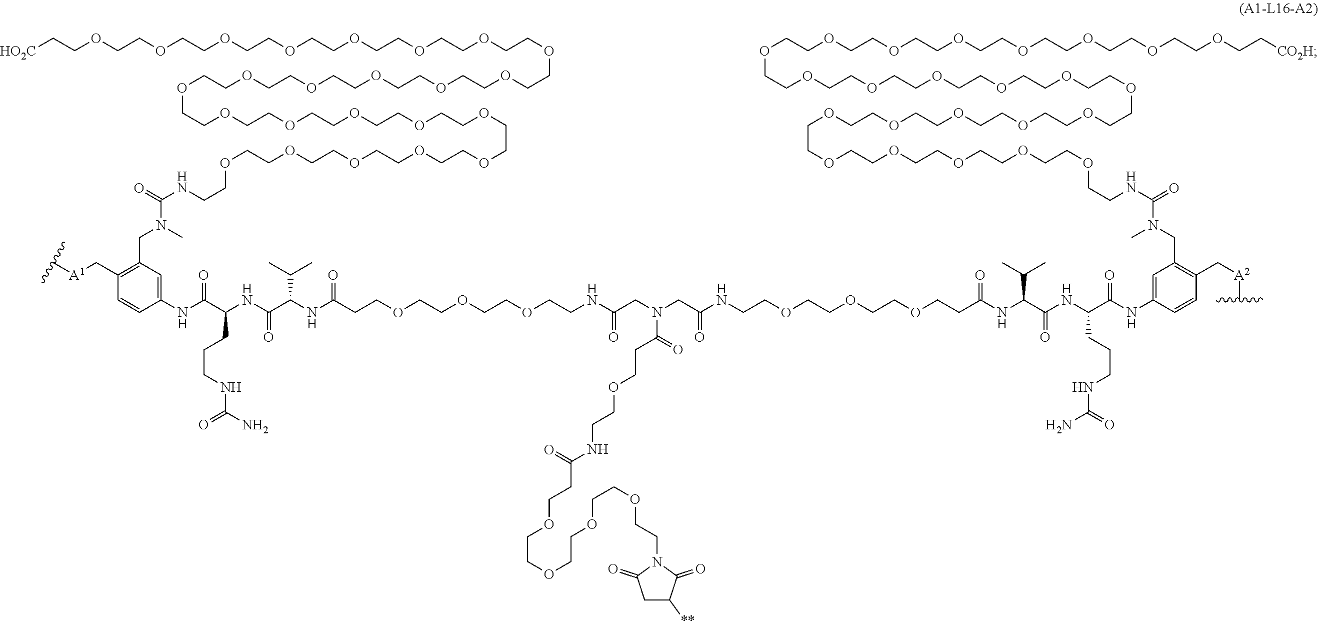

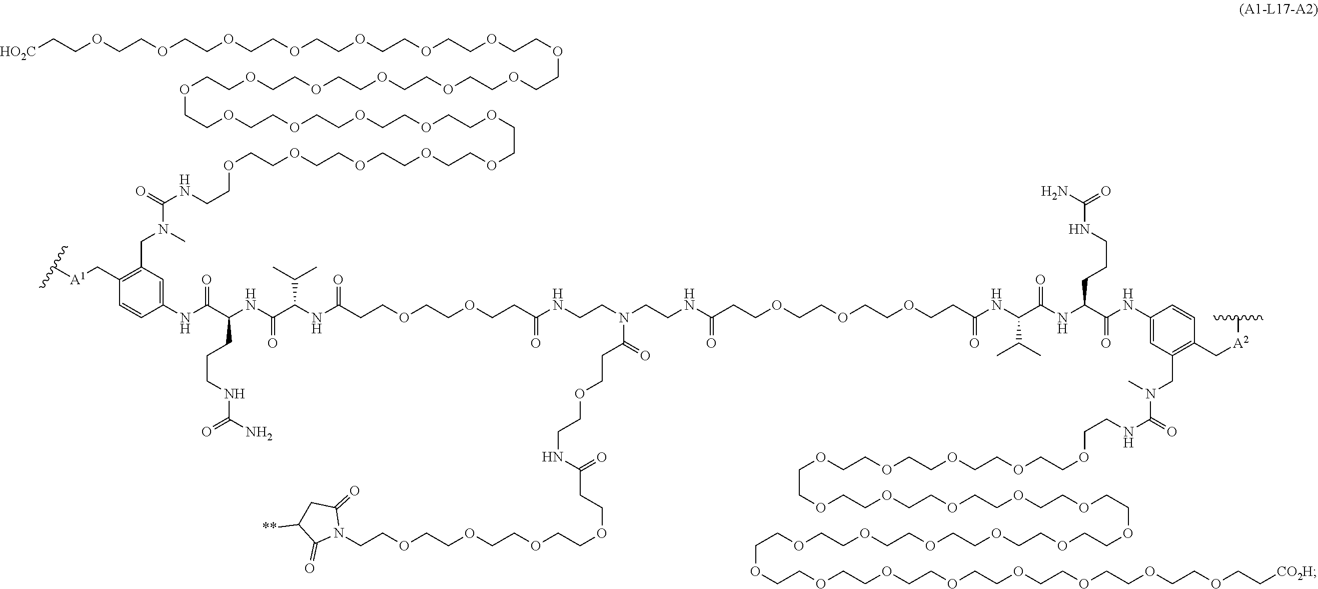

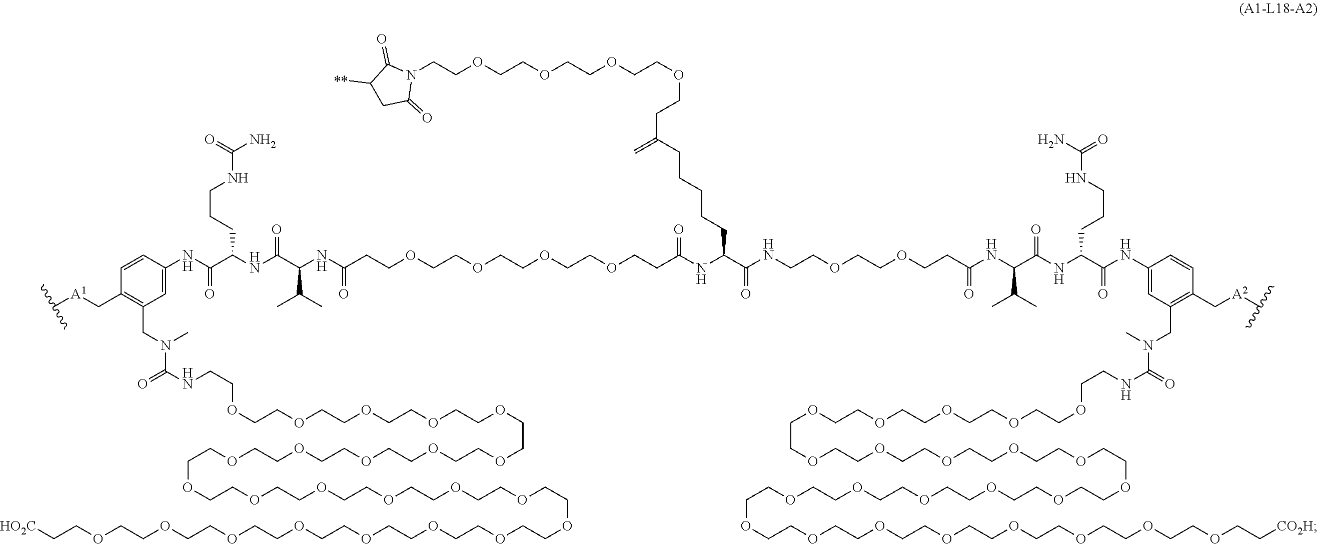

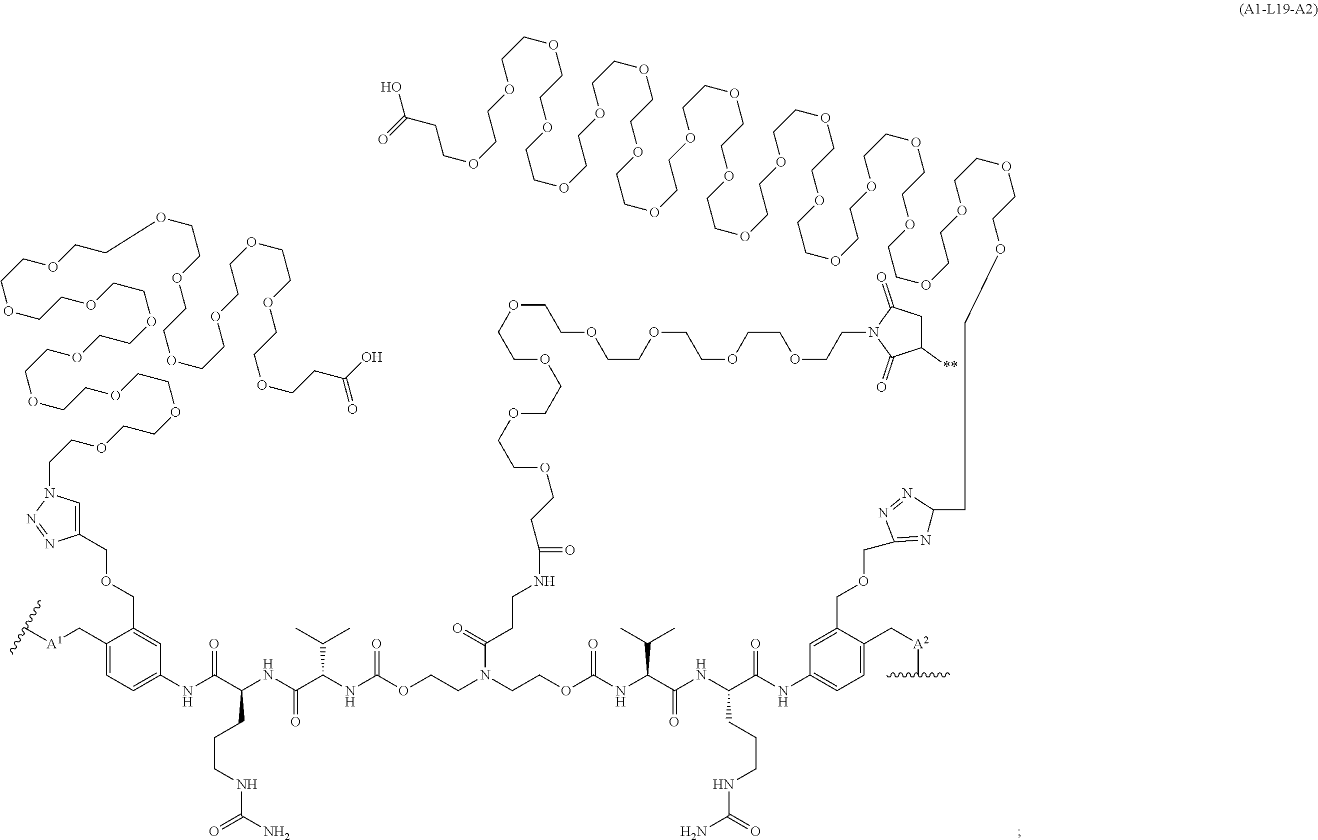

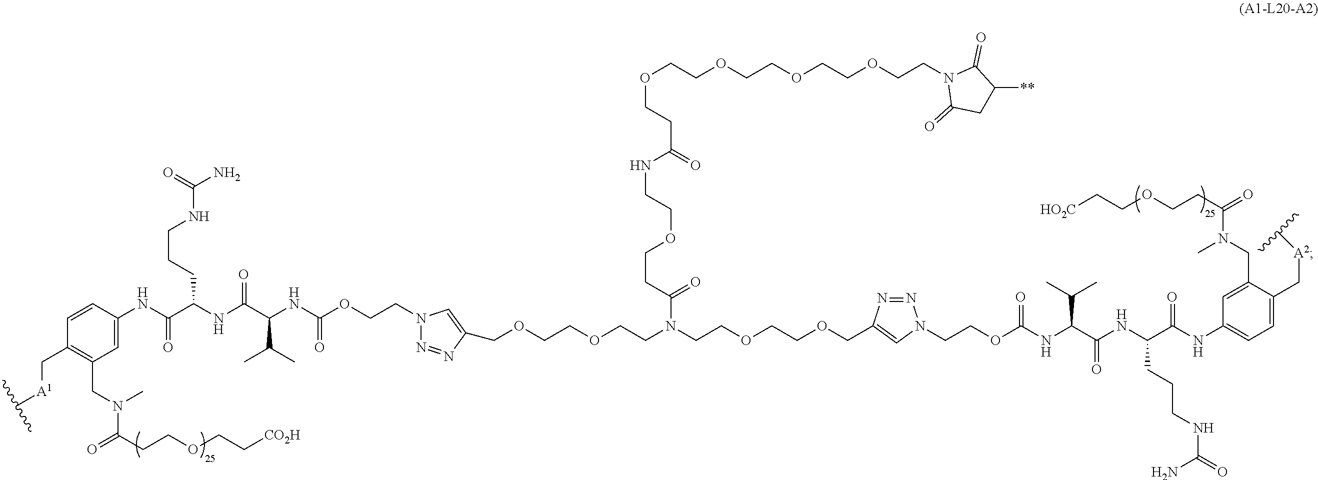

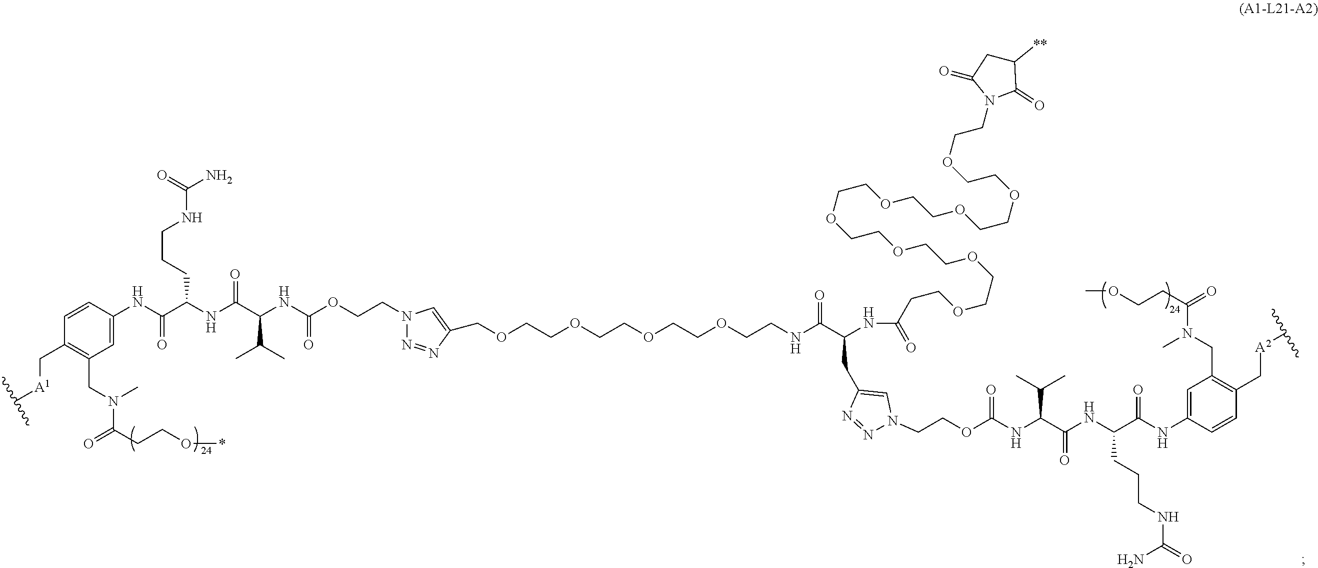

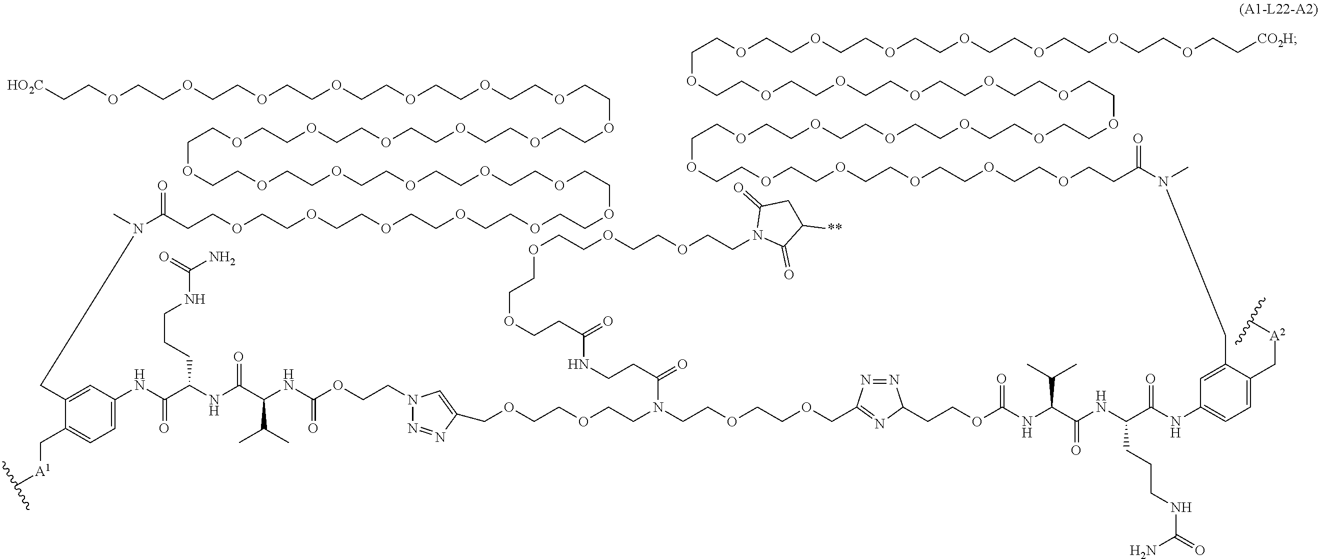

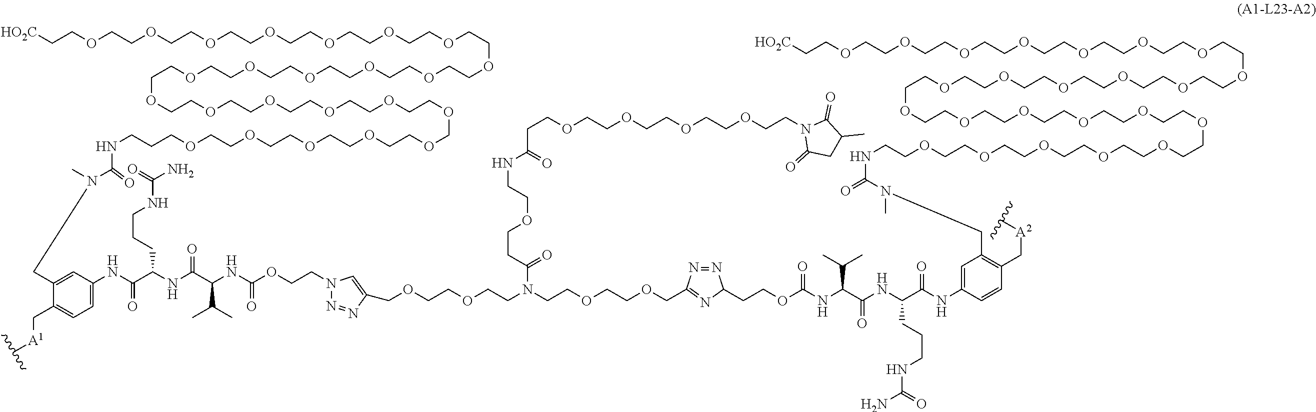

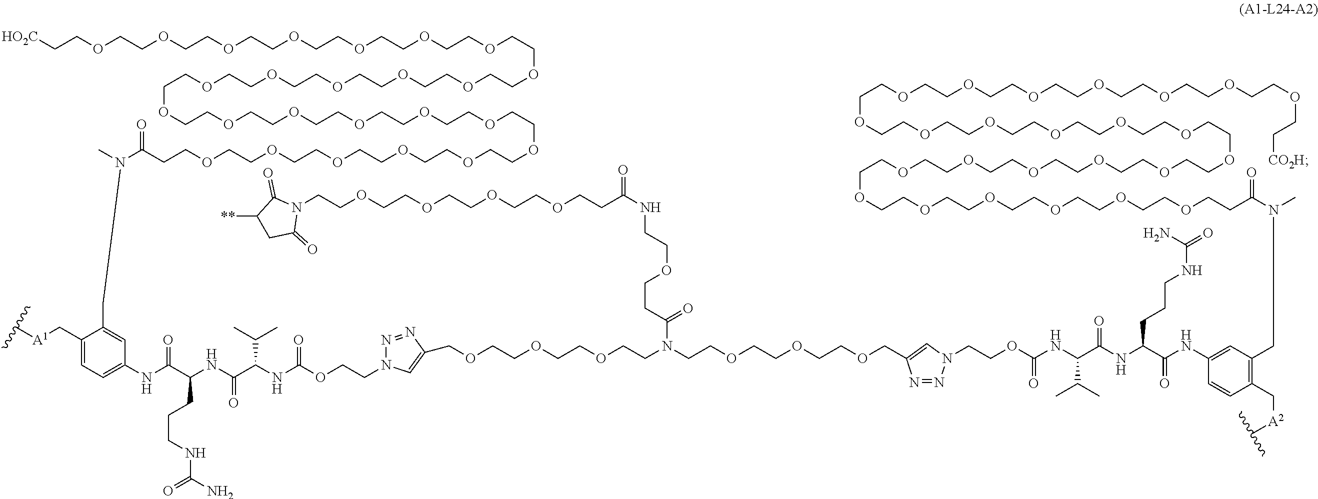

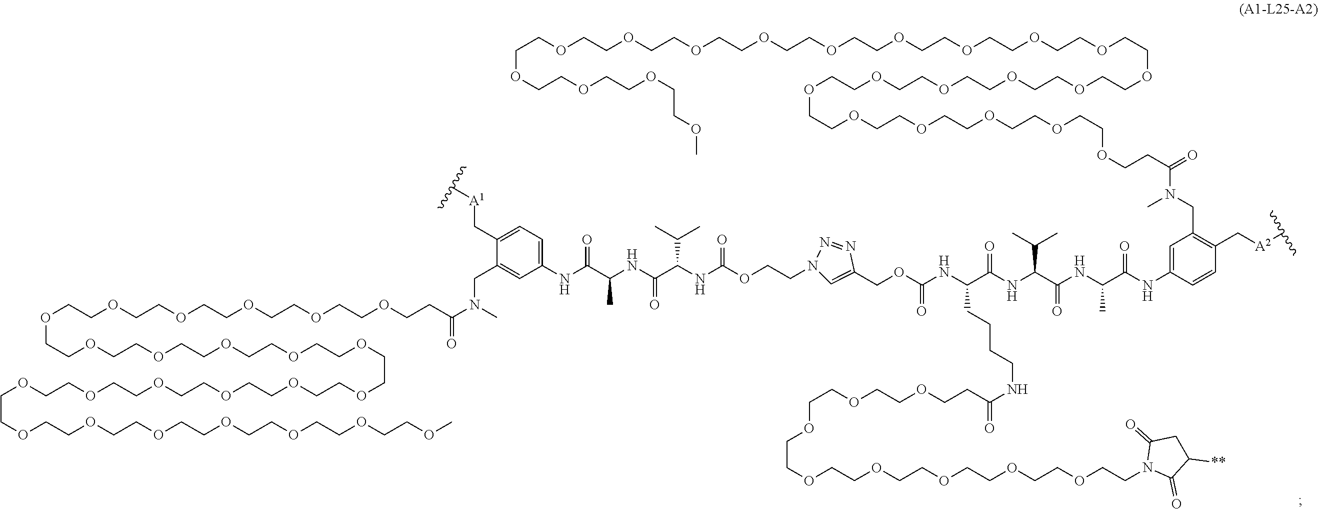

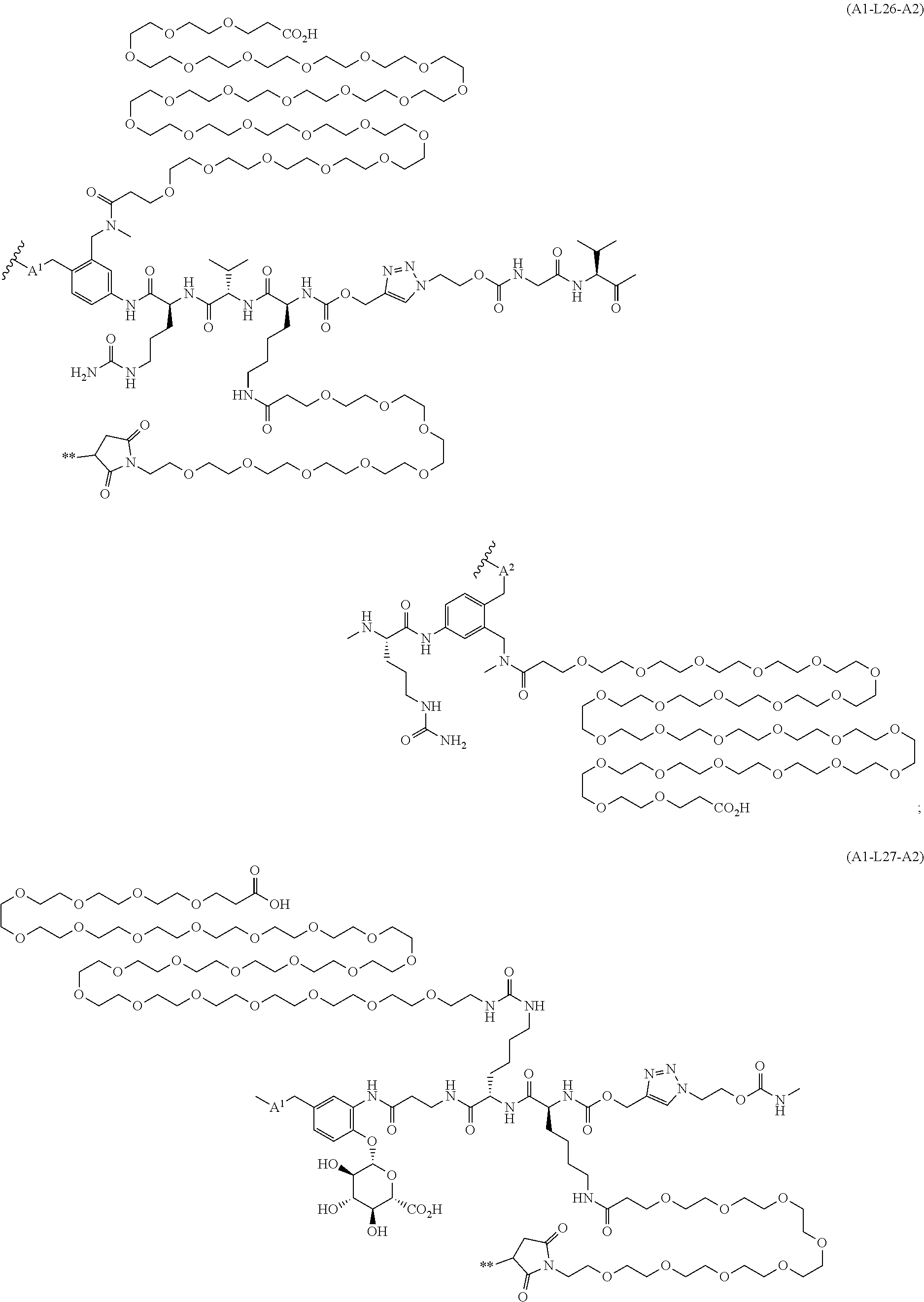

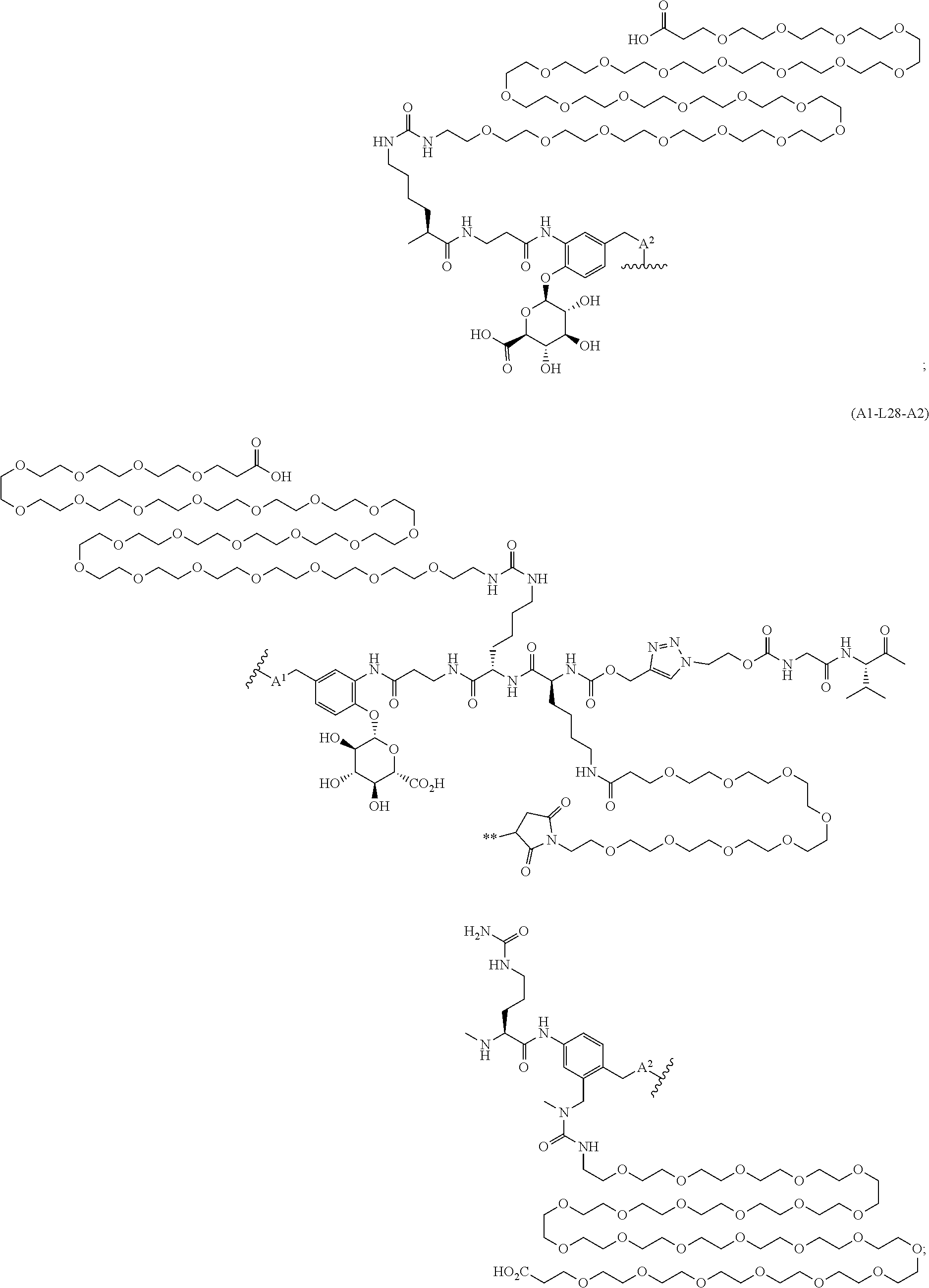

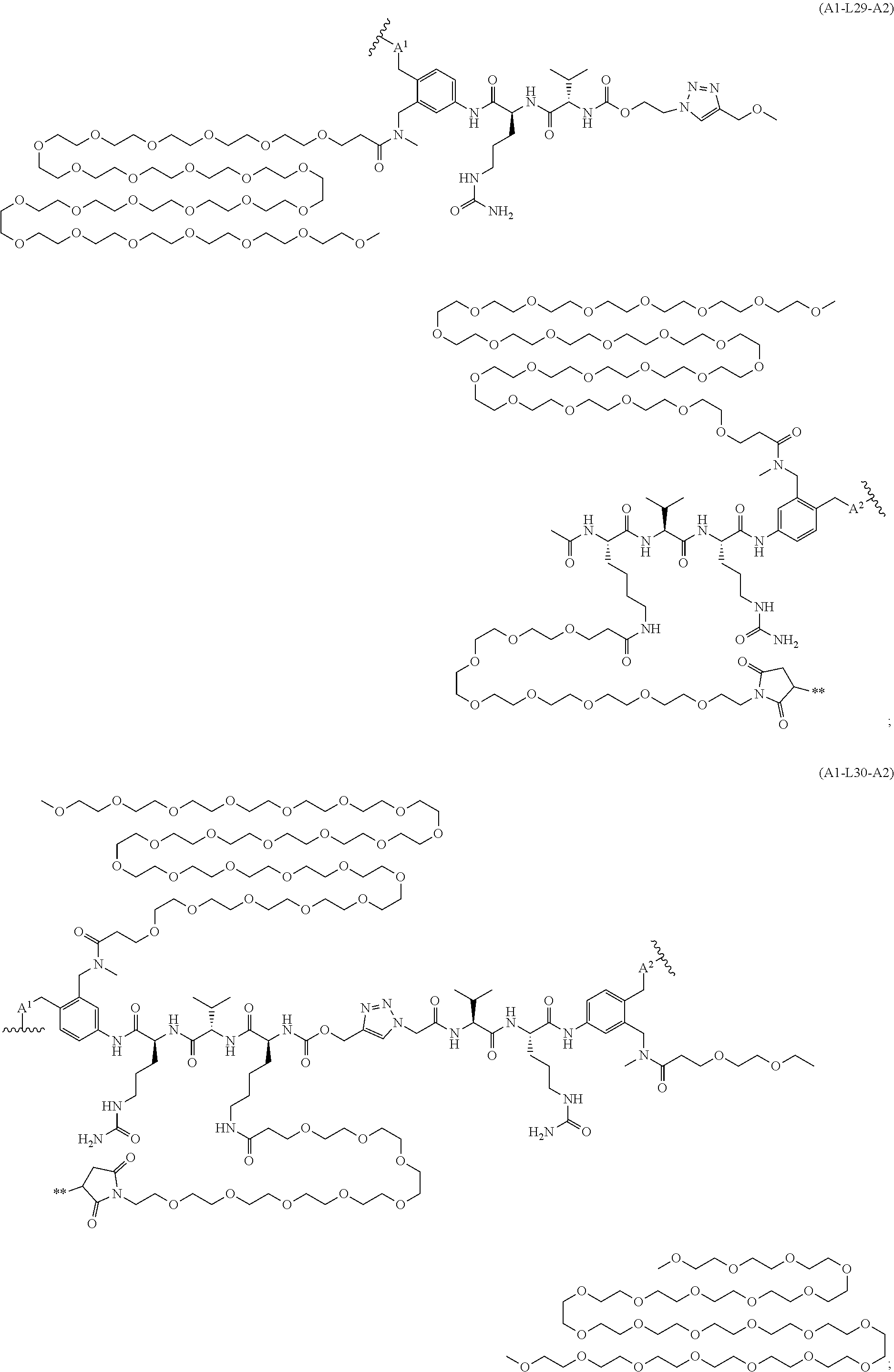

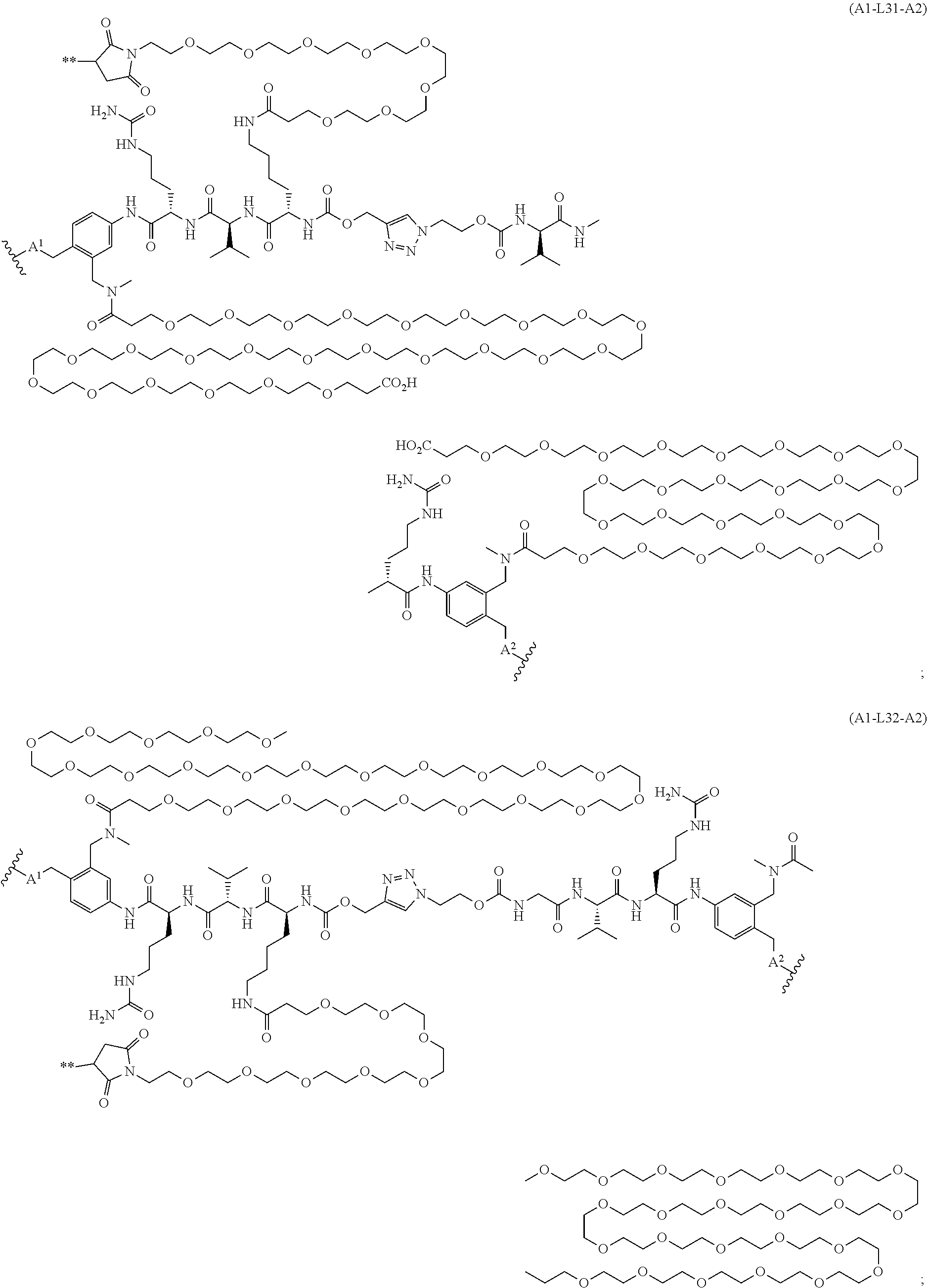

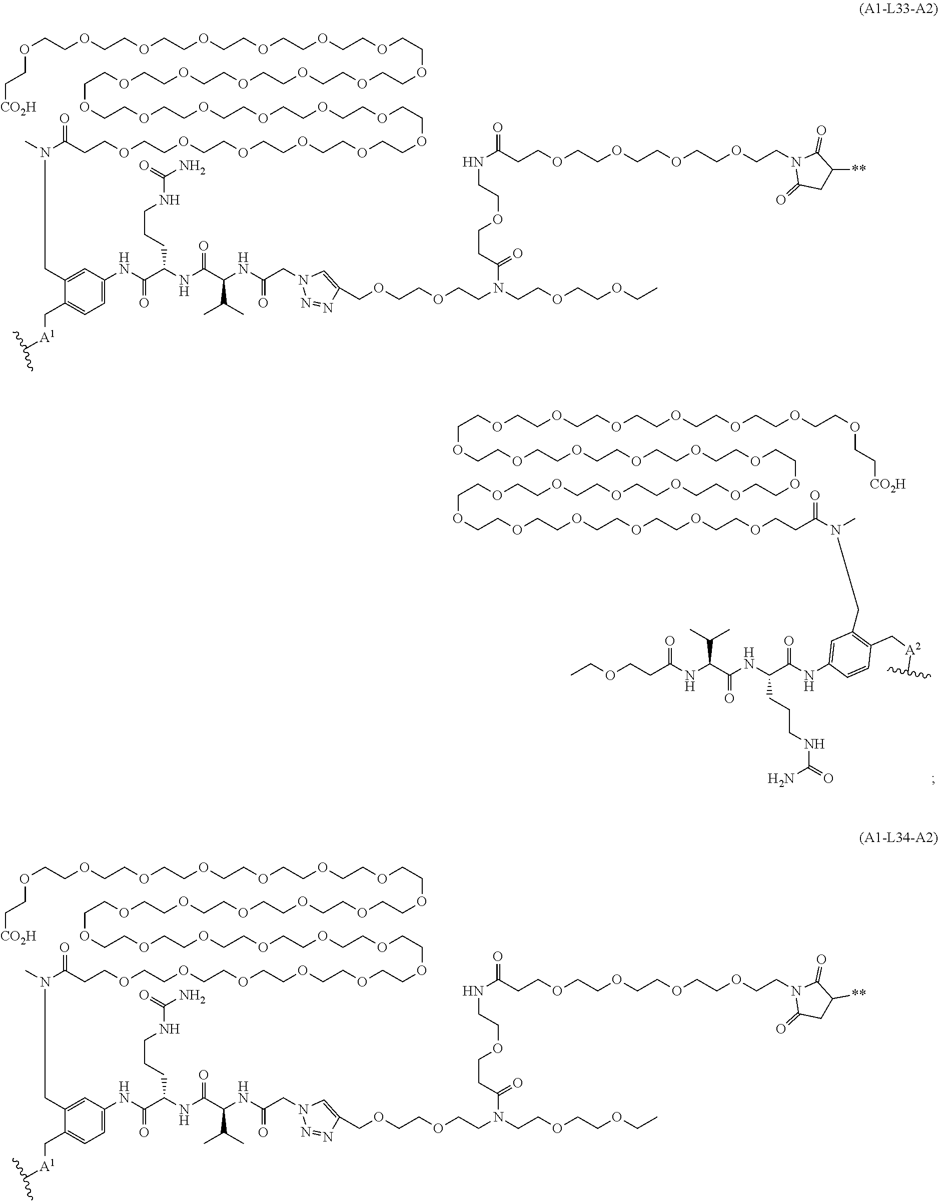

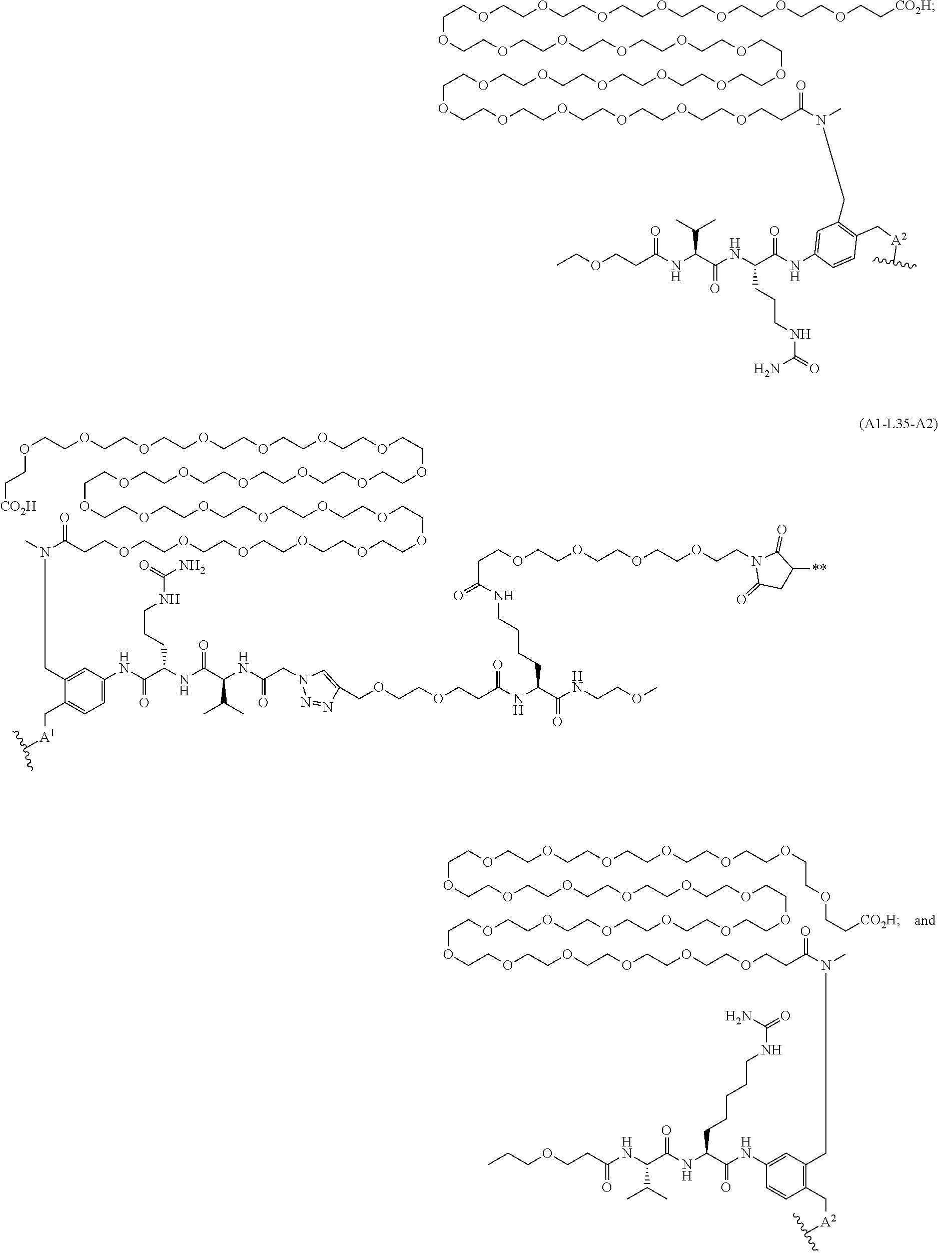

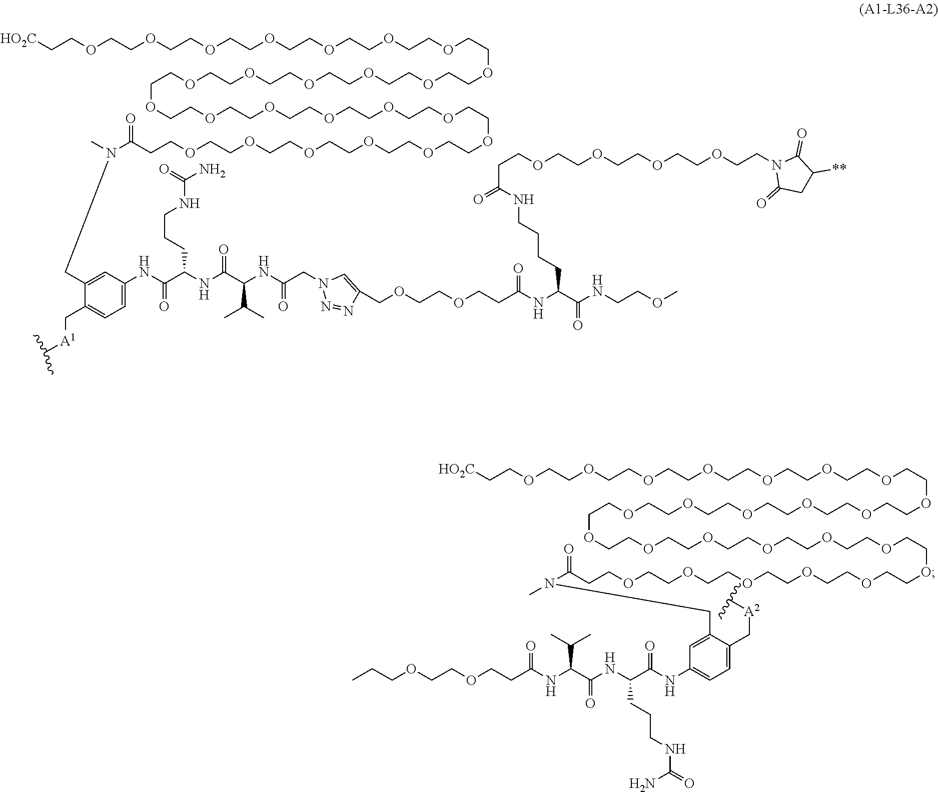

wherein A¹ and A² are each independently selected from a bond,

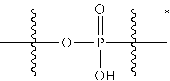 [SMILES: *OP(*)(=O)O]

and —OC(═O)—*, wherein * indicates the point of attachment to D¹ or D²; [symbol] indicates the point of attachment to the Ab; [symbol] and indicates the point of direct attachment to D¹ or D².

In an embodiment, A¹ and A² are each independently selected from a bond and —O—C(═O)—*, wherein * in A¹ and A² indicates the point of attachment to D¹ or D².

In some embodiments, the present disclosure provides, in part, novel antibody-drug conjugate (ADC) compounds with biological activity against cancer cells. The compounds may slow, inhibit, and/or reverse tumor growth in mammals, and/or may be useful for treating human cancer patients. The present disclosure more specifically relates, in some embodiments, to ADC compounds that are capable of binding and killing cancer cells. In some embodiments, the ADC compounds disclosed herein comprise a dual linker that attaches two a pharmaceutically active drugs, optionally with the proviso that neither is a BH3 mimetic, to a full-length antibody or an antigen-binding fragment. In some embodiments, the ADC compounds are also capable of internalizing into a target cell after binding.

In some embodiments, the antibody or antigen-binding fragment binds to a target antigen on a cancer cell. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor —like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74, EphA2, TROP2, B7-H3 or 5T4.

In some embodiments, the antibody or antigen-binding fragment are antibodies or antigen-binding fragments disclosed on the internet at go.drugbank.com/drugs/DB00002, in international application publication WO2018/098306, WO2016/179257, WO2011/097627, WO2017/214282, WO2017/214301, WO2017/214233, WO2013/126810, WO2008/056833, WO2020/236817, WO2017/214335, and WO2012147713, and in U.S. Pat. No. 6,870,034B2, which are incorporated by reference in their entireties.

In some embodiments, the antibody or antigen-binding fragment is an anti-HER2 antibody or antigen-binding fragment (e.g., trastuzumab or Ab T). In some embodiments, the antibody or antigen-binding fragment is an anti-CD74 antibody or antigen-binding fragment (e.g., VHmil×VK1aNQAb or milatuzumab). In some embodiments, the antibody or antigen-binding fragment is an anti-CD48 antibody or antigen-binding fragment (e.g., SGN-CD48A (MEM/MEM102 or NY920). In some embodiments, the antibody or antigen-binding fragment is an anti-PCAD antibody or antigen-binding fragment (e.g., CQY679). In some embodiments, the antibody or antigen-binding fragment is an anti-EphA2 antibody or antigen-binding fragment (e.g., 1C1). In some embodiments, the antibody or antigen-binding fragment is an antibody or antigen-binding fragment having CDR sequences is selected from those in Tables D3 and D8, or the antibody or antigen-binding fragment having variable regions is selected from those in Tables D2 and D8, or the antibody or antigen-binding fragment having full length is selected from those in Tables D4, D5, and D7.

Also provided herein, in some embodiments, are compositions comprising multiple copies of an antibody-drug conjugate (e.g., any of the exemplary antibody-drug conjugates described herein). In some embodiments, the average p of the antibody-drug conjugates in the composition is from about 2 to about 4.

In an embodiment, the antibody or antigen-binding fragment thereof is conjugated to a group selected from any one of In an embodiment, the compound or pharmaceutically acceptable salt thereof is selected from the group consisting of:

P1-L1-P1, P1-L1-P2, P1-L1-P3, P1-L1-P4, P1-L1-P5, P1-L1-P6, P1-L1-P7, P1-L1-P8, P1-L2-P1, P1-L2-P2, P1-L2-P3, P1-L2-P4, P1-L2-P5, P1-L2-P6, P1-L2-P7, P1-L2-P8, P1-L3-P1, P1-L3-P2, P1-L3-P3, P1-L3-P4, P1-L3-P5, P1-L3-P6, P1-L3-P7, P1-L3-P8, P1-L4-P1, P1-L4-P2, P1-L4-P3, P1-L4-P4, P1-L4-P5, P1-L4-P6, P1-L4-P7, P1-L4-P8, P1-L5-P1, P1-L5-P2, P1-L5-P3, P1-L5-P4, P1-L5-P5, P1-L5-P6, P1-L5-P7, P1-L5-P8, P1-L6-P1, P1-L6-P2, P1-L6-P3, P1-L6-P4, P1-L6-P5, P1-L6-P6, P1-L6-P7, P1-L6-P8, P1-L7-P1, P1-L7-P2, P1-L7-P3, P1-L7-P4, P1-L7-P5, P1-L7-P6, P1-L7-P7, P1-L7-P8, P1-L8-P1, P1-L8-P2, P1-L8-P3, P1-L8-P4, P1-L8-P5, P1-L8-P6, P1-L8-P7, P1-L8-P8, P1-L9-P1, P1-L9-P2, P1-L9-P3, P1-L9-P4, P1-L9-P5, P1-L9-P6, P1-L9-P7, P1-L9-P8, P1-L10-P1, P1-L10-P2, P1-10-P3, P1-L10-P4, P1-10-P5, P1-L10-P6, P1-L10-P7, P1-10-P8, P1-L11-P1, P1-L11-P2, P1-L11-P3, P1-L11-P4, P1-L11-P5, P1-L11-P6, P1-L11-P7, P1-L11-P8, P1-L12-P1, P1-L12-P2, P1-L12-P3, P1-L12-P4, P1-L12-P5, P1-L12-P6, P1-L12-P7, P1-L12-P8, P1-L13-P1, P1-L13-P2, P1-L13-P3, P1-L13-P4, P1-L13-P5, P1-L13-P6, P1-L13-P7, P1-L13-P8, P1-L14-P1, P1-L14-P2, P1-L14-P3, P1-L14-P4, P1-L14-P5, P1-L14-P6, P1-L14-P7, P1-L14-P8, P1-L15-P1, P1-L15-P2, P1-L15-P3, P1-L15-P4, P1-L15-P5, P1-L15-P6, P1-L15-P7, P1-L15-P8, P1-L16-P1, P1-L16-P2, P1-L16-P3, P1-L16-P4, P1-L16-P5, P1-L16-P6, P1-L16-P7, P1-L16-P8, P1-L17-P1, P1-L17-P2, P1-L17-P3, P1-L17-P4, P1-L17-P5, P1-L17-P6, P1-L17-P7, P1-L17-P8, P1-L18-P1, P1-L18-P2, P1-L18-P3, P1-L18-P4, P1-L18-P5, P1-L18-P6, P1-L18-P7, P1-L18-P8, P1-L19-P1, P1-L19-P2, P1-L19-P3, P1-L19-P4, P1-L19-P5, P1-L19-P6, P1-L19-P7, P1-L19-P8, P1-L20-P1, P1-L20-P2, P1-L20-P3, P1-L20-P4, P1-L20-P5, P1-L20-P6, P1-L20-P7, P1-L20-P8, P1-L21-P1, P1-L21-P2, P1-L21-P3, P1-L21-P4, P1-L21-P5, P1-L21-P6, P1-L21-P7, P1-L21-P8, P1-L22-P1, P1-L22-P2, P1-L22-P3, P1-L22-P4, P1-L22-P5, P1-L22-P6, P1-L22-P7, P1-L22-P8, P1-L23-P1, P1-L23-P2, P1-L23-P3, P1-L23-P4, P1-L23-P5, P1-L23-P6, P1-L23-P7, P1-L23-P8, P1-L24-P1, P1-L24-P2, P1-L24-P3, P1-L24-P4, P1-L24-P5, P1-L24-P6, P1-L24-P7, P1-L24-P8, P1-L25-P1, P1-L25-P2, P1-L25-P3, P1-L25-P4, P1-L25-P5, P1-L25-P6, P1-L25-P7, P1-L25-P8, P1-L26-P1, P1-L26-P2, P1-L26-P3, P1-L26-P4, P1-L26-P5, P1-L26-P6, P1-L26-P7, P1-L26-P8, P1-L27-P1, P1-L27-P2, P1-L27-P3, P1-L27-P4, P1-L27-P5, P1-L27-P6, P1-L27-P7, P1-L27-P8, P1-L28-P1, P1-L28-P2, P1-L28-P3, P1-L28-P4, P1-L28-P5, P1-L28-P6, P1-L28-P7, P1-L28-P8, P1-L29-P1, P1-L29-P2, P1-L29-P3, P1-L29-P4, P1-L29-P5, P1-L29-P6, P1-L29-P7, P1-L29-P8, P1-L30-P1, P1-L30-P2, P1-L30-P3, P1-L30-P4, P1-L30-P5, P1-L30-P6, P1-L30-P7, P1-L30-P8, P1-L31-P1, P1-L31-P2, P1-L31-P3, P1-L31-P4, P1-L31-P5, P1-L31-P6, P1-L31-P7, P1-L31-P8, P1-L32-P1, P1-L32-P2, P1-L32-P3, P1-L32-P4, P1-L32-P5, P1-L32-P6, P1-L32-P7, P1-L32-P8, P1-L33-P1, P1-L33-P2, P1-L33-P3, P1-L33-P4, P1-L33-P5, P1-L33-P6, P1-L33-P7, P1-L33-P8, P1-L34-P1, P1-L34-P2, P1-L34-P3, P1-L34-P4, P1-L34-P5, P1-L34-P6, P1-L34-P7, P1-L34-P8, P1-L35-P1, P1-L35-P2, P1-L35-P3, P1-L35-P4, P1-L35-P5, P1-L35-P6, P1-L35-P7, P1-L35-P8, P1-L36-P1, P1-L36-P2, P1-L36-P3, P1-L36-P4, P1-L36-P5, P1-L36-P6, P1-L36-P7, P1-L36-P8, P2-L1-P1, P2-L1-P2, P2-L1-P3, P2-L1-P4, P2-L1-P5, P2-L1-P6, P2-L1-P7, P2-L1-P8, P2-L2-P1, P2-L2-P2, P2-L2-P3, P2-L2-P4, P2-L2-P5, P2-L2-P6, P2-L2-P7, P2-L2-P8, P2-L3-P1, P2-L3-P2, P2-L3-P3, P2-L3-P4, P2-L3-P5, P2-L3-P6, P2-L3-P7, P2-L3-P8, P2-L4-P1, P2-L4-P2, P2-L4-P3, P2-L4-P4, P2-L4-P5, P2-L4-P6, P2-L4-P7, P2-L4-P8, P2-L5-P1, P2-L5-P2, P2-L5-P3, P2-L5-P4, P2-L5-P5, P2-L5-P6, P2-L5-P7, P2-L5-P8, P2-L6-P1, P2-L6-P2, P2-L6-P3, P2-L6-P4, P2-L6-P5, P2-L6-P6, P2-L6-P7, P2-L6-P8, P2-L7-P1, P2-L7-P2, P2-L7-P3, P2-L7-P4, P2-L7-P5, P2-L7-P6, P2-L7-P7, P2-L7-P8, P2-L8-P1, P2-L8-P2, P2-L8-P3, P2-L8-P4, P2-L8-P5, P2-L8-P6, P2-L8-P7, P2-L8-P8, P2-L9-P1, P2-L9-P2, P2-L9-P3, P2-L9-P4, P2-L9-P5, P2-L9-P6, P2-L9-P7, P2-L9-P8, P2-L10-P1, P2-L10-P2, P2-L10-P3, P2-L10-P4, P2-L10-P5, P2-L10-P6, P2-L10-P7, P2-10-P8, P2-L11-P1, P2-L11-P2, P2-L11-P3, P2-L11-P4, P2-L11-P5, P2-L11-P6, P2-L11-P7, P2-L11-P8, P2-L12-P1, P2-L12-P2, P2-L12-P3, P2-L12-P4, P2-L12-P5, P2-L12-P6, P2-L12-P7, P2-L12-P8, P2-L13-P1, P2-L13-P2, P2-L13-P3, P2-L13-P4, P2-L13-P5, P2-L13-P6, P2-L13-P7, P2-L13-P8, P2-L14-P1, P2-L14-P2, P2-L14-P3, P2-L14-P4, P2-L14-P5, P2-L14-P6, P2-L14-P7, P2-L14-P8, P2-L15-P1, P2-L15-P2, P2-L15-P3, P2-L15-P4, P2-L15-P5, P2-L15-P6, P2-L15-P7, P2-L15-P8, P2-L16-P1, P2-L16-P2, P2-L16-P3, P2-L16-P4, P2-L16-P5, P2-L16-P6, P2-L16-P7, P2-L16-P8, P2-L17-P1, P2-L17-P2, P2-L17-P3, P2-L17-P4, P2-L17-P5, P2-L17-P6, P2-L17-P7, P2-L17-P8, P2-L18-P1, P2-L18-P2, P2-L18-P3, P2-L18-P4, P2-L18-P5, P2-L18-P6, P2-L18-P7, P2-L18-P8, P2-L19-P1, P2-L19-P2, P2-L19-P3, P2-L19-P4, P2-L19-P5, P2-L19-P6, P2-L19-P7, P2-L19-P8, P2-L20-P1, P2-L20-P2, P2-L20-P3, P2-L20-P4, P2-L20-P5, P2-L20-P6, P2-L20-P7, P2-L20-P8, P2-L21-P1, P2-L21-P2, P2-L21-P3, P2-L21-P4, P2-L21-P5, P2-L21-P6, P2-L21-P7, P2-L21-P8, P2-L22-P1, P2-L22-P2, P2-L22-P3, P2-L22-P4, P2-L22-P5, P2-L22-P6, P2-L22-P7, P2-L22-P8, P2-L23-P1, P2-L23-P2, P2-L23-P3, P2-L23-P4, P2-L23-P5, P2-L23-P6, P2-L23-P7, P2-L23-P8, P2-L24-P1, P2-L24-P2, P2-L24-P3, P2-L24-P4, P2-L24-P5, P2-L24-P6, P2-L24-P7, P2-L24-P8, P2-L25-P1, P2-L25-P2, P2-L25-P3, P2-L25-P4, P2-L25-P5, P2-L25-P6, P2-L25-P7, P2-L25-P8, P2-L26-P1, P2-L26-P2, P2-L26-P3, P2-L26-P4, P2-L26-P5, P2-L26-P6, P2-L26-P7, P2-L26-P8, P2-L27-P1, P2-L27-P2, P2-L27-P3, P2-L27-P4, P2-L27-P5, P2-L27-P6, P2-L27-P7, P2-L27-P8, P2-L28-P1, P2-L28-P2, P2-L28-P3, P2-L28-P4, P2-L28-P5, P2-L28-P6, P2-L28-P7, P2-L28-P8, P2-L29-P1, P2-L29-P2, P2-L29-P3, P2-L29-P4, P2-L29-P5, P2-L29-P6, P2-L29-P7, P2-L29-P8, P2-L30-P1, P2-L30-P2, P2-L30-P3, P2-L30-P4, P2-L30-P5, P2-L30-P6, P2-L30-P7, P2-L30-P8, P2-L31-P1, P2-L31-P2, P2-L31-P3, P2-L31-P4, P2-L31-P5, P2-L31-P6, P2-L31-P7, P2-L31-P8, P2-L32-P1, P2-L32-P2, P2-L32-P3, P2-L32-P4, P2-L32-P5, P2-L32-P6, P2-L32-P7, P2-L32-P8, P2-L33-P1, P2-L33-P2, P2-L33-P3, P2-L33-P4, P2-L33-P5, P2-L33-P6, P2-L33-P7, P2-L33-P8, P2-L34-P1, P2-L34-P2, P2-L34-P3, P2-L34-P4, P2-L34-P5, P2-L34-P6, P2-L34-P7, P2-L34-P8, P2-L35-P1, P2-L35-P2, P2-L35-P3, P2-L35-P4, P2-L35-P5, P2-L35-P6, P2-L35-P7, P2-L35-P8, P2-L36-P1, P2-L36-P2, P2-L36-P3, P2-L36-P4, P2-L36-P5, P2-L36-P6, P2-L36-P7, P2-L36-P8, P3-L1-P1, P3-L1-P2, P3-L1-P3, P3-L1-P4, P3-L1-P5, P3-L1-P6, P3-L1-P7, P3-L1-P8, P3-L2-P1, P3-L2-P2, P3-L2-P3, P3-L2-P4, P3-L2-P5, P3-L2-P6, P3-L2-P7, P3-L2-P8, P3-L3-P1, P3-L3-P2, P3-L3-P3, P3-L3-P4, P3-L3-P5, P3-L3-P6, P3-L3-P7, P3-L3-P8, P3-L4-P1, P3-L4-P2, P3-L4-P3, P3-L4-P4, P3-L4-P5, P3-L4-P6, P3-L4-P7, P3-L4-P8, P3-L5-P1, P3-L5-P2, P3-L5-P3, P3-L5-P4, P3-L5-P5, P3-L5-P6, P3-L5-P7, P3-L5-P8, P3-L6-P1, P3-L6-P2, P3-L6-P3, P3-L6-P4, P3-L6-P5, P3-L6-P6, P3-L6-P7, P3-L6-P8, P3-L7-P1, P3-L7-P2, P3-L7-P3, P3-L7-P4, P3-L7-P5, P3-L7-P6, P3-L7-P7, P3-L7-P8, P3-L8-P1, P3-L8-P2, P3-L8-P3, P3-L8-P4, P3-L8-P5, P3-L8-P6, P3-L8-P7, P3-L8-P8, P3-L9-P1, P3-L9-P2, P3-L9-P3, P3-L9-P4, P3-L9-P5, P3-L9-P6, P3-L9-P7, P3-L9-P8, P3-L10-P1, P3-10-P2, P3-10-P3, P3-L10-P4, P3-10-P5, P3-10-P6, P3-10-P7, P3-L10-P8, P3-L11-P1, P3-L11-P2, P3-L11-P3, P3-L11-P4, P3-L11-P5, P3-L11-P6, P3-L11-P7, P3-L11-P8, P3-L12-P1, P3-L12-P2, P3-L12-P3, P3-L12-P4, P3-L12-P5, P3-L12-P6, P3-L12-P7, P3-L12-P8, P3-L13-P1, P3-L13-P2, P3-L13-P3, P3-L13-P4, P3-L13-P5, P3-L13-P6, P3-L13-P7, P3-L13-P8, P3-L14-P1, P3-L14-P2, P3-L14-P3, P3-L14-P4, P3-L14-P5, P3-L14-P6, P3-L14-P7, P3-L14-P8, P3-L15-P1, P3-L15-P2, P3-L15-P3, P3-L15-P4, P3-L15-P5, P3-L15-P6, P3-L15-P7, P3-L15-P8, P3-L16-P1, P3-L16-P2, P3-L16-P3, P3-L16-P4, P3-L16-P5, P3-L16-P6, P3-L16-P7, P3-L16-P8, P3-L17-P1, P3-L17-P2, P3-L17-P3, P3-L17-P4, P3-L17-P5, P3-L17-P6, P3-L17-P7, P3-L17-P8, P3-L18-P1, P3-L18-P2, P3-L18-P3, P3-L18-P4, P3-L18-P5, P3-L18-P6, P3-L18-P7, P3-L18-P8, P3-L19-P1, P3-L19-P2, P3-L19-P3, P3-L19-P4, P3-L19-P5, P3-L19-P6, P3-L19-P7, P3-L19-P8, P3-L20-P1, P3-L20-P2, P3-L20-P3, P3-L20-P4, P3-L20-P5, P3-L20-P6, P3-L20-P7, P3-L20-P8, P3-L21-P1, P3-L21-P2, P3-L21-P3, P3-L21-P4, P3-L21-P5, P3-L21-P6, P3-L21-P7, P3-L21-P8, P3-L22-P1, P3-L22-P2, P3-L22-P3, P3-L22-P4, P3-L22-P5, P3-L22-P6, P3-L22-P7, P3-L22-P8, P3-L23-P1, P3-L23-P2, P3-L23-P3, P3-L23-P4, P3-L23-P5, P3-L23-P6, P3-L23-P7, P3-L23-P8, P3-L24-P1, P3-L24-P2, P3-L24-P3, P3-L24-P4, P3-L24-P5, P3-L24-P6, P3-L24-P7, P3-L24-P8, P3-L25-P1, P3-L25-P2, P3-L25-P3, P3-L25-P4, P3-L25-P5, P3-L25-P6, P3-L25-P7, P3-L25-P8, P3-L26-P1, P3-L26-P2, P3-L26-P3, P3-L26-P4, P3-L26-P5, P3-L26-P6, P3-L26-P7, P3-L26-P8, P3-L27-P1, P3-L27-P2, P3-L27-P3, P3-L27-P4, P3-L27-P5, P3-L27-P6, P3-L27-P7, P3-L27-P8, P3-L28-P1, P3-L28-P2, P3-L28-P3, P3-L28-P4, P3-L28-P5, P3-L28-P6, P3-L28-P7, P3-L28-P8, P3-L29-P1, P3-L29-P2, P3-L29-P3, P3-L29-P4, P3-L29-P5, P3-L29-P6, P3-L29-P7, P3-L29-P8, P3-L30-P1, P3-L30-P2, P3-L30-P3, P3-L30-P4, P3-L30-P5, P3-L30-P6, P3-L30-P7, P3-L30-P8, P3-L31-P1, P3-L31-P2, P3-L31-P3, P3-L31-P4, P3-L31-P5, P3-L31-P6, P3-L31-P7, P3-L31-P8, P3-L32-P1, P3-L32-P2, P3-L32-P3, P3-L32-P4, P3-L32-P5, P3-L32-P6, P3-L32-P7, P3-L32-P8, P3-L33-P1, P3-L33-P2, P3-L33-P3, P3-L33-P4, P3-L33-P5, P3-L33-P6, P3-L33-P7, P3-L33-P8, P3-L34-P1, P3-L34-P2, P3-L34-P3, P3-L34-P4, P3-L34-P5, P3-L34-P6, P3-L34-P7, P3-L34-P8, P3-L35-P1, P3-L35-P2, P3-L35-P3, P3-L35-P4, P3-L35-P5, P3-L35-P6, P3-L35-P7, P3-L35-P8, P3-L36-P1, P3-L36-P2, P3-L36-P3, P3-L36-P4, P3-L36-P5, P3-L36-P6, P3-L36-P7, P3-L36-P8, P4-L1-P1, P4-L1-P2, P4-L1-P3, P4-L1-P4, P4-L1-P5, P4-L1-P6, P4-L1-P7, P4-L1-P8, P4-L2-P1, P4-L2-P2, P4-L2-P3, P4-L2-P4, P4-L2-P5, P4-L2-P6, P4-L2-P7, P4-L2-P8, P4-L3-P1, P4-L3-P2, P4-L3-P3, P4-L3-P4, P4-L3-P5, P4-L3-P6, P4-L3-P7, P4-L3-P8, P4-L4-P1, P4-L4-P2, P4-L4-P3, P4-L4-P4, P4-L4-P5, P4-L4-P6, P4-L4-P7, P4-L4-P8, P4-L5-P1, P4-L5-P2, P4-L5-P3, P4-L5-P4, P4-L5-P5, P4-L5-P6, P4-L5-P7, P4-L5-P8, P4-L6-P1, P4-L6-P2, P4-L6-P3, P4-L6-P4, P4-L6-P5, P4-L6-P6, P4-L6-P7, P4-L6-P8, P4-L7-P1, P4-L7-P2, P4-L7-P3, P4-L7-P4, P4-L7-P5, P4-L7-P6, P4-L7-P7, P4-L7-P8, P4-L8-P1, P4-L8-P2, P4-L8-P3, P4-L8-P4, P4-L8-P5, P4-L8-P6, P4-L8-P7, P4-L8-P8, P4-L9-P1, P4-L9-P2, P4-L9-P3, P4-L9-P4, P4-L9-P5, P4-L9-P6, P4-L9-P7, P4-L9-P8, P4-10-P1, P4-10-P2, P4-10-P3, P4-L10-P4, P4-L10-P5, P4-10-P6, P4-10-P7, P4-10-P8, P4-11-P1, P4-L11-P2, P4-L11-P3, P4-L11-P4, P4-L11-P5, P4-L11-P6, P4-L11-P7, P4-L11-P8, P4-L12-P1, P4-L12-P2, P4-L12-P3, P4-L12-P4, P4-L12-P5, P4-L12-P6, P4-L12-P7, P4-L12-P8, P4-L13-P1, P4-L13-P2, P4-L13-P3, P4-L13-P4, P4-L13-P5, P4-L13-P6, P4-L13-P7, P4-L13-P8, P4-L14-P1, P4-L14-P2, P4-L14-P3, P4-L14-P4, P4-L14-P5, P4-L14-P6, P4-L14-P7, P4-L14-P8, P4-L15-P1, P4-L15-P2, P4-L15-P3, P4-L15-P4, P4-L15-P5, P4-L15-P6, P4-L15-P7, P4-L15-P8, P4-L16-P1, P4-L16-P2, P4-L16-P3, P4-L16-P4, P4-L16-P5, P4-L16-P6, P4-L16-P7, P4-L16-P8, P4-L17-P1, P4-L17-P2, P4-L17-P3, P4-L17-P4, P4-L17-P5, P4-L17-P6, P4-L17-P7, P4-L17-P8, P4-L18-P1, P4-L18-P2, P4-L18-P3, P4-L18-P4, P4-L18-P5, P4-L18-P6, P4-L18-P7, P4-L18-P8, P4-L19-P1, P4-L19-P2, P4-L19-P3, P4-L19-P4, P4-L19-P5, P4-L19-P6, P4-L19-P7, P4-L19-P8, P4-L20-P1, P4-L20-P2, P4-L20-P3, P4-L20-P4, P4-L20-P5, P4-L20-P6, P4-L20-P7, P4-L20-P8, P4-L21-P1, P4-L21-P2, P4-L21-P3, P4-L21-P4, P4-L21-P5, P4-L21-P6, P4-L21-P7, P4-L21-P8, P4-L22-P1, P4-L22-P2, P4-L22-P3, P4-L22-P4, P4-L22-P5, P4-L22-P6, P4-L22-P7, P4-L22-P8, P4-L23-P1, P4-L23-P2, P4-L23-P3, P4-L23-P4, P4-L23-P5, P4-L23-P6, P4-L23-P7, P4-L23-P8, P4-L24-P1, P4-L24-P2, P4-L24-P3, P4-L24-P4, P4-L24-P5, P4-L24-P6, P4-L24-P7, P4-L24-P8, P4-L25-P1, P4-L25-P2, P4-L25-P3, P4-L25-P4, P4-L25-P5, P4-L25-P6, P4-L25-P7, P4-L25-P8, P4-L26-P1, P4-L26-P2, P4-L26-P3, P4-L26-P4, P4-L26-P5, P4-L26-P6, P4-L26-P7, P4-L26-P8, P4-L27-P1, P4-L27-P2, P4-L27-P3, P4-L27-P4, P4-L27-P5, P4-L27-P6, P4-L27-P7, P4-L27-P8, P4-L28-P1, P4-L28-P2, P4-L28-P3, P4-L28-P4, P4-L28-P5, P4-L28-P6, P4-L28-P7, P4-L28-P8, P4-L29-P1, P4-L29-P2, P4-L29-P3, P4-L29-P4, P4-L29-P5, P4-L29-P6, P4-L29-P7, P4-L29-P8, P4-L30-P1, P4-L30-P2, P4-L30-P3, P4-L30-P4, P4-L30-P5, P4-L30-P6, P4-L30-P7, P4-L30-P8, P4-L31-P1, P4-L31-P2, P4-L31-P3, P4-L31-P4, P4-L31-P5, P4-L31-P6, P4-L31-P7, P4-L31-P8, P4-L32-P1, P4-L32-P2, P4-L32-P3, P4-L32-P4, P4-L32-P5, P4-L32-P6, P4-L32-P7, P4-L32-P8, P4-L33-P1, P4-L33-P2, P4-L33-P3, P4-L33-P4, P4-L33-P5, P4-L33-P6, P4-L33-P7, P4-L33-P8, P4-L34-P1, P4-L34-P2, P4-L34-P3, P4-L34-P4, P4-L34-P5, P4-L34-P6, P4-L34-P7, P4-L34-P8, P4-L35-P1, P4-L35-P2, P4-L35-P3, P4-L35-P4, P4-L35-P5, P4-L35-P6, P4-L35-P7, P4-L35-P8, P4-L36-P1, P4-L36-P2, P4-L36-P3, P4-L36-P4, P4-L36-P5, P4-L36-P6, P4-L36-P7, P4-L36-P8, P5-L1-P1, P5-L1-P2, P5-L1-P3, P5-L1-P4, P5-L1-P5, P5-L1-P6, P5-L1-P7, P5-L1-P8, P5-L2-P1, P5-L2-P2, P5-L2-P3, P5-L2-P4, P5-L2-P5, P5-L2-P6, P5-L2-P7, P5-L2-P8, P5-L3-P1, P5-L3-P2, P5-L3-P3, P5-L3-P4, P5-L3-P5, P5-L3-P6, P5-L3-P7, P5-L3-P8, P5-L4-P1, P5-L4-P2, P5-L4-P3, P5-L4-P4, P5-L4-P5, P5-L4-P6, P5-L4-P7, P5-L4-P8, P5-L5-P1, P5-L5-P2, P5-L5-P3, P5-L5-P4, P5-L5-P5, P5-L5-P6, P5-L5-P7, P5-L5-P8, P5-L6-P1, P5-L6-P2, P5-L6-P3, P5-L6-P4, P5-L6-P5, P5-L6-P6, P5-L6-P7, P5-L6-P8, P5-L7-P1, P5-L7-P2, P5-L7-P3, P5-L7-P4, P5-L7-P5, P5-L7-P6, P5-L7-P7, P5-L7-P8, P5-L8-P1, P5-L8-P2, P5-L8-P3, P5-L8-P4, P5-L8-P5, P5-L8-P6, P5-L8-P7, P5-L8-P8, P5-L9-P1, P5-L9-P2, P5-L9-P3, P5-L9-P4, P5-L9-P5, P5-L9-P6, P5-L9-P7, P5-L9-P8, P5-10-P1, P5-110-P2, P5-10-P3, P5-L10-P4, P5-10-P5, P5-10-P6, P5-10-P7, P5-10-P8, P5-L11-P1, P5-L11-P2, P5-L11-P3, P5-L11-P4, P5-L11-P5, P5-L11-P6, P5-L11-P7, P5-L11-P8, P5-L12-P1, P5-L12-P2, P5-L12-P3, P5-L12-P4, P5-L12-P5, P5-L12-P6, P5-L12-P7, P5-L12-P8, P5-L13-P1, P5-L13-P2, P5-L13-P3, P5-L13-P4, P5-L13-P5, P5-L13-P6, P5-L13-P7, P5-L13-P8, P5-L14-P1, P5-L14-P2, P5-L14-P3, P5-L14-P4, P5-L14-P5, P5-L14-P6, P5-L14-P7, P5-L14-P8, P5-15-P1, P5-L15-P2, P5-L15-P3, P5-L15-P4, P5-L15-P5, P5-L15-P6, P5-L15-P7, P5-L15-P8, P5-L16-P1, P5-L16-P2, P5-L16-P3, P5-L16-P4, P5-L16-P5, P5-L16-P6, P5-L16-P7, P5-L16-P8, P5-L17-P1, P5-L17-P2, P5-L17-P3, P5-L17-P4, P5-L17-P5, P5-L17-P6, P5-L17-P7, P5-L17-P8, P5-L18-P1, P5-L18-P2, P5-L18-P3, P5-L18-P4, P5-L18-P5, P5-L18-P6, P5-L18-P7, P5-L18-P8, P5-L19-P1, P5-L19-P2, P5-L19-P3, P5-L19-P4, P5-L19-P5, P5-L19-P6, P5-L19-P7, P5-L19-P8, P5-L20-P1, P5-L20-P2, P5-L20-P3, P5-L20-P4, P5-L20-P5, P5-L20-P6, P5-L20-P7, P5-L20-P8, P5-L21-P1, P5-L21-P2, P5-L21-P3, P5-L21-P4, P5-L21-P5, P5-L21-P6, P5-L21-P7, P5-L21-P8, P5-L22-P1, P5-L22-P2, P5-L22-P3, P5-L22-P4, P5-L22-P5, P5-L22-P6, P5-L22-P7, P5-L22-P8, P5-L23-P1, P5-L23-P2, P5-L23-P3, P5-L23-P4, P5-L23-P5, P5-L23-P6, P5-L23-P7, P5-L23-P8, P5-L24-P1, P5-L24-P2, P5-L24-P3, P5-L24-P4, P5-L24-P5, P5-L24-P6, P5-L24-P7, P5-L24-P8, P5-L25-P1, P5-L25-P2, P5-L25-P3, P5-L25-P4, P5-L25-P5, P5-L25-P6, P5-L25-P7, P5-L25-P8, P5-L26-P1, P5-L26-P2, P5-L26-P3, P5-L26-P4, P5-L26-P5, P5-L26-P6, P5-L26-P7, P5-L26-P8, P5-L27-P1, P5-L27-P2, P5-L27-P3, P5-L27-P4, P5-L27-P5, P5-L27-P6, P5-L27-P7, P5-L27-P8, P5-L28-P1, P5-L28-P2, P5-L28-P3, P5-L28-P4, P5-L28-P5, P5-L28-P6, P5-L28-P7, P5-L28-P8, P5-L29-P1, P5-L29-P2, P5-L29-P3, P5-L29-P4, P5-L29-P5, P5-L29-P6, P5-L29-P7, P5-L29-P8, P5-L30-P1, P5-L30-P2, P5-L30-P3, P5-L30-P4, P5-L30-P5, P5-L30-P6, P5-L30-P7, P5-L30-P8, P5-L31-P1, P5-L31-P2, P5-L31-P3, P5-L31-P4, P5-L31-P5, P5-L31-P6, P5-L31-P7, P5-L31-P8, P5-L32-P1, P5-L32-P2, P5-L32-P3, P5-L32-P4, P5-L32-P5, P5-L32-P6, P5-L32-P7, P5-L32-P8, P5-L33-P1, P5-L33-P2, P5-L33-P3, P5-L33-P4, P5-L33-P5, P5-L33-P6, P5-L33-P7, P5-L33-P8, P5-L34-P1, P5-L34-P2, P5-L34-P3, P5-L34-P4, P5-L34-P5, P5-L34-P6, P5-L34-P7, P5-L34-P8, P5-L35-P1, P5-L35-P2, P5-L35-P3, P5-L35-P4, P5-L35-P5, P5-L35-P6, P5-L35-P7, P5-L35-P8, P5-L36-P1, P5-L36-P2, P5-L36-P3, P5-L36-P4, P5-L36-P5, P5-L36-P6, P5-L36-P7, P5-L36-P8, P6-L1-P1, P6-L1-P2, P6-L1-P3, P6-L1-P4, P6-L1-P5, P6-L1-P6, P6-L1-P7, P6-L1-P8, P6-L2-P1, P6-L2-P2, P6-L2-P3, P6-L2-P4, P6-L2-P5, P6-L2-P6, P6-L2-P7, P6-L2-P8, P6-L3-P1, P6-L3-P2, P6-L3-P3, P6-L3-P4, P6-L3-P5, P6-L3-P6, P6-L3-P7, P6-L3-P8, P6-L4-P1, P6-L4-P2, P6-L4-P3, P6-L4-P4, P6-L4-P5, P6-L4-P6, P6-L4-P7, P6-L4-P8, P6-L5-P1, P6-L5-P2, P6-L5-P3, P6-L5-P4, P6-L5-P5, P6-L5-P6, P6-L5-P7, P6-L5-P8, P6-L6-P1, P6-L6-P2, P6-L6-P3, P6-L6-P4, P6-L6-P5, P6-L6-P6, P6-L6-P7, P6-L6-P8, P6-L7-P1, P6-L7-P2, P6-L7-P3, P6-L7-P4, P6-L7-P5, P6-L7-P6, P6-L7-P7, P6-L7-P8, P6-L8-P1, P6-L8-P2, P6-L8-P3, P6-L8-P4, P6-L8-P5, P6-L8-P6, P6-L8-P7, P6-L8-P8, P6-L9-P1, P6-L9-P2, P6-L9-P3, P6-L9-P4, P6-L9-P5, P6-L9-P6, P6-L9-P7, P6-L9-P8, P6-10-P1, P6-10-P2, P6-10-P3, P6-L10-P4, P6-10-P5, P6-10-P6, P6-L10-P7, P6-10-P8, P6-11-P1, P6-L11-P2, P6-L11-P3, P6-L11-P4, P6-L11-P5, P6-L11-P6, P6-L11-P7, P6-L11-P8, P6-L12-P1, P6-L12-P2, P6-L12-P3, P6-L12-P4, P6-L12-P5, P6-L12-P6, P6-L12-P7, P6-L12-P8, P6-L13-P1, P6-L13-P2, P6-L13-P3, P6-L13-P4, P6-L13-P5, P6-L13-P6, P6-L13-P7, P6-L13-P8, P6-L14-P1, P6-L14-P2, P6-L14-P3, P6-L14-P4, P6-L14-P5, P6-L14-P6, P6-L14-P7, P6-L14-P8, P6-L15-P1, P6-L15-P2, P6-L15-P3, P6-L15-P4, P6-L15-P5, P6-L15-P6, P6-L15-P7, P6-L15-P8, P6-L16-P1, P6-L16-P2, P6-L16-P3, P6-L16-P4, P6-L16-P5, P6-L16-P6, P6-L16-P7, P6-L16-P8, P6-L17-P1, P6-L17-P2, P6-L17-P3, P6-L17-P4, P6-L17-P5, P6-L17-P6, P6-L17-P7, P6-L17-P8, P6-L18-P1, P6-L18-P2, P6-L18-P3, P6-L18-P4, P6-L18-P5, P6-L18-P6, P6-L18-P7, P6-L18-P8, P6-L19-P1, P6-L19-P2, P6-L19-P3, P6-L19-P4, P6-L19-P5, P6-L19-P6, P6-L19-P7, P6-L19-P8, P6-L20-P1, P6-L20-P2, P6-L20-P3, P6-L20-P4, P6-L20-P5, P6-L20-P6, P6-L20-P7, P6-L20-P8, P6-L21-P1, P6-L21-P2, P6-L21-P3, P6-L21-P4, P6-L21-P5, P6-L21-P6, P6-L21-P7, P6-L21-P8, P6-L22-P1, P6-L22-P2, P6-L22-P3, P6-L22-P4, P6-L22-P5, P6-L22-P6, P6-L22-P7, P6-L22-P8, P6-L23-P1, P6-L23-P2, P6-L23-P3, P6-L23-P4, P6-L23-P5, P6-L23-P6, P6-L23-P7, P6-L23-P8, P6-L24-P1, P6-L24-P2, P6-L24-P3, P6-L24-P4, P6-L24-P5, P6-L24-P6, P6-L24-P7, P6-L24-P8, P6-L25-P1, P6-L25-P2, P6-L25-P3, P6-L25-P4, P6-L25-P5, P6-L25-P6, P6-L25-P7, P6-L25-P8, P6-L26-P1, P6-L26-P2, P6-L26-P3, P6-L26-P4, P6-L26-P5, P6-L26-P6, P6-L26-P7, P6-L26-P8, P6-L27-P1, P6-L27-P2, P6-L27-P3, P6-L27-P4, P6-L27-P5, P6-L27-P6, P6-L27-P7, P6-L27-P8, P6-L28-P1, P6-L28-P2, P6-L28-P3, P6-L28-P4, P6-L28-P5, P6-L28-P6, P6-L28-P7, P6-L28-P8, P6-L29-P1, P6-L29-P2, P6-L29-P3, P6-L29-P4, P6-L29-P5, P6-L29-P6, P6-L29-P7, P6-L29-P8, P6-L30-P1, P6-L30-P2, P6-L30-P3, P6-L30-P4, P6-L30-P5, P6-L30-P6, P6-L30-P7, P6-L30-P8, P6-L31-P1, P6-L31-P2, P6-L31-P3, P6-L31-P4, P6-L31-P5, P6-L31-P6, P6-L31-P7, P6-L31-P8, P6-L32-P1, P6-L32-P2, P6-L32-P3, P6-L32-P4, P6-L32-P5, P6-L32-P6, P6-L32-P7, P6-L32-P8, P6-L33-P1, P6-L33-P2, P6-L33-P3, P6-L33-P4, P6-L33-P5, P6-L33-P6, P6-L33-P7, P6-L33-P8, P6-L34-P1, P6-L34-P2, P6-L34-P3, P6-L34-P4, P6-L34-P5, P6-L34-P6, P6-L34-P7, P6-L34-P8, P6-L35-P1, P6-L35-P2, P6-L35-P3, P6-L35-P4, P6-L35-P5, P6-L35-P6, P6-L35-P7, P6-L35-P8, P6-L36-P1, P6-L36-P2, P6-L36-P3, P6-L36-P4, P6-L36-P5, P6-L36-P6, P6-L36-P7, P6-L36-P8, P7-L1-P1, P7-L1-P2, P7-L1-P3, P7-L1-P4, P7-L1-P5, P7-L1-P6, P7-L1-P7, P7-L1-P8, P7-L2-P1, P7-L2-P2, P7-L2-P3, P7-L2-P4, P7-L2-P5, P7-L2-P6, P7-L2-P7, P7-L2-P8, P7-L3-P1, P7-L3-P2, P7-L3-P3, P7-L3-P4, P7-L3-P5, P7-L3-P6, P7-L3-P7, P7-L3-P8, P7-L4-P1, P7-L4-P2, P7-L4-P3, P7-L4-P4, P7-L4-P5, P7-L4-P6, P7-L4-P7, P7-L4-P8, P7-L5-P1, P7-L5-P2, P7-L5-P3, P7-L5-P4, P7-L5-P5, P7-L5-P6, P7-L5-P7, P7-L5-P8, P7-L6-P1, P7-L6-P2, P7-L6-P3, P7-L6-P4, P7-L6-P5, P7-L6-P6, P7-L6-P7, P7-L6-P8, P7-L7-P1, P7-L7-P2, P7-L7-P3, P7-L7-P4, P7-L7-P5, P7-L7-P6, P7-L7-P7, P7-L7-P8, P7-L8-P1, P7-L8-P2, P7-L8-P3, P7-L8-P4, P7-L8-P5, P7-L8-P6, P7-L8-P7, P7-L8-P8, P7-L9-P1, P7-L9-P2, P7-L9-P3, P7-L9-P4, P7-L9-P5, P7-L9-P6, P7-L9-P7, P7-L9-P8, P7-10-P1, P7-10-P2, P7-10-P3, P7-110-P4, P7-10-P5, P7-10-P6, P7-10-P7, P7-10-P8, P7-L11-P1, P7-L11-P2, P7-L11-P3, P7-L11-P4, P7-L11-P5, P7-L11-P6, P7-L11-P7, P7-L11-P8, P7-L12-P1, P7-L12-P2, P7-L12-P3, P7-L12-P4, P7-L12-P5, P7-L12-P6, P7-L12-P7, P7-L12-P8, P7-L13-P1, P7-L13-P2, P7-L13-P3, P7-L13-P4, P7-L13-P5, P7-L13-P6, P7-L13-P7, P7-L13-P8, P7-L14-P1, P7-L14-P2, P7-L14-P3, P7-L14-P4, P7-L14-P5, P7-L14-P6, P7-L14-P7, P7-L14-P8, P7-L15-P1, P7-L15-P2, P7-L15-P3, P7-L15-P4, P7-L15-P5, P7-L15-P6, P7-L15-P7, P7-L15-P8, P7-L16-P1, P7-L16-P2, P7-L16-P3, P7-L16-P4, P7-L16-P5, P7-L16-P6, P7-L16-P7, P7-L16-P8, P7-L17-P1, P7-L17-P2, P7-L17-P3, P7-L17-P4, P7-L17-P5, P7-L17-P6, P7-L17-P7, P7-L17-P8, P7-L18-P1, P7-L18-P2, P7-L18-P3, P7-L18-P4, P7-L18-P5, P7-L18-P6, P7-L18-P7, P7-L18-P8, P7-L19-P1, P7-L19-P2, P7-L19-P3, P7-L19-P4, P7-L19-P5, P7-L19-P6, P7-L19-P7, P7-L19-P8, P7-L20-P1, P7-L20-P2, P7-L20-P3, P7-L20-P4, P7-L20-P5, P7-L20-P6, P7-L20-P7, P7-L20-P8, P7-L21-P1, P7-L21-P2, P7-L21-P3, P7-L21-P4, P7-L21-P5, P7-L21-P6, P7-L21-P7, P7-L21-P8, P7-L22-P1, P7-L22-P2, P7-L22-P3, P7-L22-P4, P7-L22-P5, P7-L22-P6, P7-L22-P7, P7-L22-P8, P7-L23-P1, P7-L23-P2, P7-L23-P3, P7-L23-P4, P7-L23-P5, P7-L23-P6, P7-L23-P7, P7-L23-P8, P7-L24-P1, P7-L24-P2, P7-L24-P3, P7-L24-P4, P7-L24-P5, P7-L24-P6, P7-L24-P7, P7-L24-P8, P7-L25-P1, P7-L25-P2, P7-L25-P3, P7-L25-P4, P7-L25-P5, P7-L25-P6, P7-L25-P7, P7-L25-P8, P7-L26-P1, P7-L26-P2, P7-L26-P3, P7-L26-P4, P7-L26-P5, P7-L26-P6, P7-L26-P7, P7-L26-P8, P7-L27-P1, P7-L27-P2, P7-L27-P3, P7-L27-P4, P7-L27-P5, P7-L27-P6, P7-L27-P7, P7-L27-P8, P7-L28-P1, P7-L28-P2, P7-L28-P3, P7-L28-P4, P7-L28-P5, P7-L28-P6, P7-L28-P7, P7-L28-P8, P7-L29-P1, P7-L29-P2, P7-L29-P3, P7-L29-P4, P7-L29-P5, P7-L29-P6, P7-L29-P7, P7-L29-P8, P7-L30-P1, P7-L30-P2, P7-L30-P3, P7-L30-P4, P7-L30-P5, P7-L30-P6, P7-L30-P7, P7-L30-P8, P7-L31-P1, P7-L31-P2, P7-L31-P3, P7-L31-P4, P7-L31-P5, P7-L31-P6, P7-L31-P7, P7-L31-P8, P7-L32-P1, P7-L32-P2, P7-L32-P3, P7-L32-P4, P7-L32-P5, P7-L32-P6, P7-L32-P7, P7-L32-P8, P7-L33-P1, P7-L33-P2, P7-L33-P3, P7-L33-P4, P7-L33-P5, P7-L33-P6, P7-L33-P7, P7-L33-P8, P7-L34-P1, P7-L34-P2, P7-L34-P3, P7-L34-P4, P7-L34-P5, P7-L34-P6, P7-L34-P7, P7-L34-P8, P7-L35-P1, P7-L35-P2, P7-L35-P3, P7-L35-P4, P7-L35-P5, P7-L35-P6, P7-L35-P7, P7-L35-P8, P7-L36-P1, P7-L36-P2, P7-L36-P3, P7-L36-P4, P7-L36-P5, P7-L36-P6, P7-L36-P7, P7-L36-P8, P8-L1-P1, P8-L1-P2, P8-L1-P3, P8-L1-P4, P8-L1-P5, P8-L1-P6, P8-L1-P7, P8-L1-P8, P8-L2-P1, P8-L2-P2, P8-L2-P3, P8-L2-P4, P8-L2-P5, P8-L2-P6, P8-L2-P7, P8-L2-P8, P8-L3-P1, P8-L3-P2, P8-L3-P3, P8-L3-P4, P8-L3-P5, P8-L3-P6, P8-L3-P7, P8-L3-P8, P8-L4-P1, P8-L4-P2, P8-L4-P3, P8-L4-P4, P8-L4-P5, P8-L4-P6, P8-L4-P7, P8-L4-P8, P8-L5-P1, P8-L5-P2, P8-L5-P3, P8-L5-P4, P8-L5-P5, P8-L5-P6, P8-L5-P7, P8-L5-P8, P8-L6-P1, P8-L6-P2, P8-L6-P3, P8-L6-P4, P8-L6-P5, P8-L6-P6, P8-L6-P7, P8-L6-P8, P8-L7-P1, P8-L7-P2, P8-L7-P3, P8-L7-P4, P8-L7-P5, P8-L7-P6, P8-L7-P7, P8-L7-P8, P8-L8-P1, P8-L8-P2, P8-L8-P3, P8-L8-P4, P8-L8-P5, P8-L8-P6, P8-L8-P7, P8-L8-P8, P8-L9-P1, P8-L9-P2, P8-L9-P3, P8-L9-P4, P8-L9-P5, P8-L9-P6, P8-L9-P7, P8-L9-P8, P8-L10-P1, P8-10-P2, P8-10-P3, P8-L10-P4, P8-10-P5, P8-10-P6, P8-10-P7, P8-10-P8, P8-L11-P1, P8-L11-P2, P8-L11-P3, P8-L11-P4, P8-L11-P5, P8-L11-P6, P8-L11-P7, P8-L11-P8, P8-L12-P1, P8-L12-P2, P8-L12-P3, P8-L12-P4, P8-L12-P5, P8-L12-P6, P8-L12-P7, P8-L12-P8, P8-L13-P1, P8-L13-P2, P8-L13-P3, P8-L13-P4, P8-L13-P5, P8-L13-P6, P8-L13-P7, P8-L13-P8, P8-L14-P1, P8-L14-P2, P8-L14-P3, P8-L14-P4, P8-L14-P5, P8-L14-P6, P8-L14-P7, P8-L14-P8, P8-L15-P1, P8-L15-P2, P8-L15-P3, P8-L15-P4, P8-L15-P5, P8-L15-P6, P8-L15-P7, P8-L15-P8, P8-L16-P1, P8-L16-P2, P8-L16-P3, P8-L16-P4, P8-L16-P5, P8-L16-P6, P8-L16-P7, P8-L16-P8, P8-L17-P1, P8-L17-P2, P8-L17-P3, P8-L17-P4, P8-L17-P5, P8-L17-P6, P8-L17-P7, P8-L17-P8, P8-L18-P1, P8-L18-P2, P8-L18-P3, P8-L18-P4, P8-L18-P5, P8-L18-P6, P8-L18-P7, P8-L18-P8, P8-L19-P1, P8-L19-P2, P8-L19-P3, P8-L19-P4, P8-L19-P5, P8-L19-P6, P8-L19-P7, P8-L19-P8, P8-L20-P1, P8-L20-P2, P8-L20-P3, P8-L20-P4, P8-L20-P5, P8-L20-P6, P8-L20-P7, P8-L20-P8, P8-L21-P1, P8-L21-P2, P8-L21-P3, P8-L21-P4, P8-L21-P5, P8-L21-P6, P8-L21-P7, P8-L21-P8, P8-L22-P1, P8-L22-P2, P8-L22-P3, P8-L22-P4, P8-L22-P5, P8-L22-P6, P8-L22-P7, P8-L22-P8, P8-L23-P1, P8-L23-P2, P8-L23-P3, P8-L23-P4, P8-L23-P5, P8-L23-P6, P8-L23-P7, P8-L23-P8, P8-L24-P1, P8-L24-P2, P8-L24-P3, P8-L24-P4, P8-L24-P5, P8-L24-P6, P8-L24-P7, P8-L24-P8, P8-L25-P1, P8-L25-P2, P8-L25-P3, P8-L25-P4, P8-L25-P5, P8-L25-P6, P8-L25-P7, P8-L25-P8, P8-L26-P1, P8-L26-P2, P8-L26-P3, P8-L26-P4, P8-L26-P5, P8-L26-P6, P8-L26-P7, P8-L26-P8, P8-L27-P1, P8-L27-P2, P8-L27-P3, P8-L27-P4, P8-L27-P5, P8-L27-P6, P8-L27-P7, P8-L27-P8, P8-L28-P1, P8-L28-P2, P8-L28-P3, P8-L28-P4, P8-L28-P5, P8-L28-P6, P8-L28-P7, P8-L28-P8, P8-L29-P1, P8-L29-P2, P8-L29-P3, P8-L29-P4, P8-L29-P5, P8-L29-P6, P8-L29-P7, P8-L29-P8, P8-L30-P1, P8-L30-P2, P8-L30-P3, P8-L30-P4, P8-L30-P5, P8-L30-P6, P8-L30-P7, P8-L30-P8, P8-L31-P1, P8-L31-P2, P8-L31-P3, P8-L31-P4, P8-L31-P5, P8-L31-P6, P8-L31-P7, P8-L31-P8, P8-L32-P1, P8-L32-P2, P8-L32-P3, P8-L32-P4, P8-L32-P5, P8-L32-P6, P8-L32-P7, P8-L32-P8, P8-L33-P1, P8-L33-P2, P8-L33-P3, P8-L33-P4, P8-L33-P5, P8-L33-P6, P8-L33-P7, P8-L33-P8, P8-L34-P1, P8-L34-P2, P8-L34-P3, P8-L34-P4, P8-L34-P5, P8-L34-P6, P8-L34-P7, P8-L34-P8, P8-L35-P1, P8-L35-P2, P8-L35-P3, P8-L35-P4, P8-L35-P5, P8-L35-P6, P8-L35-P7, P8-L35-P8, P8-L36-P1, P8-L36-P2, P8-L36-P3, P8-L36-P4, P8-L36-P5, P8-L36-P6, P8-L36-P7 and P8-L36-P8.

According to an aspect of the invention, there is hereby provided a pharmaceutical composition comprising the conjugate and a pharmaceutically acceptable carrier.

According to an aspect of the invention, there is hereby provided a method of treating a subject having or suspected of having a cancer, comprising administering to the subject a therapeutically effective amount of the conjugate or pharmaceutical composition of the invention.

In an embodiment, the cancer expresses a target antigen.

In an embodiment, the cancer is a tumor or a hematological cancer, optionally, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer.

According to an aspect of the invention, there is hereby provided a method of reducing or inhibiting the growth of a tumor in a subject, comprising administering to the subject a therapeutically effective amount of the conjugate or pharmaceutical composition of the invention.

In an embodiment, the tumor expresses a target antigen.

In an embodiment, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, spleen cancer, pancreatic cancer, stomach cancer, colon cancer, or head and neck cancer.

According to an aspect of the invention, there is hereby provided a method of reducing or inhibiting a hematological cancer in a subject, comprising administering to the subject a therapeutically effective amount of the conjugate or pharmaceutical composition of the invention.

In an embodiment, the hematological cancer expresses a target antigen.

In an embodiment, the hematological cancer is chronic lymphocytic leukemia (CLL), follicular lymphoma, mantle cell lymphoma, diffuse large B-cell lymphoma, acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), Hodgkin's lymphoma, non-Hodgkin's lymphoma or myelodysplasia syndrome (MDS).

In an embodiment, administration of the conjugate or pharmaceutical composition reduces or inhibits the growth of the tumor or hematological cancer by at least about 10%, at least about 20%, at least about 30%, at least about 40%, at least about 50%, at least about 60%, at least about 70%, at least about 80%, at least about 90%, at least about 95%, or at least about 99%.

According to an aspect of the invention, there is hereby provided a method of reducing or slowing the expansion of a cancer cell population in a subject, comprising administering to the subject a therapeutically effective amount of the conjugate or pharmaceutical composition of the invention.

In an embodiment, the cancer cell population expresses a target antigen.

In an embodiment, the cancer cell population is from a tumor or a hematological cancer, optionally wherein the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer.

In an embodiment, administration of the conjugate or pharmaceutical composition reduces the cancer cell population or slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%.

In an embodiment, the antibody-drug conjugate is administered as monotherapy. In another embodiment, the antibody-drug conjugate is administered adjunctive to another therapeutic agent or radiation therapy. In an embodiment, the antibody-drug conjugate is administered in an amount effective to sensitize the tumor cells to one or more additional therapeutic agents and/or radiation therapy.

In an embodiment, the method further comprises administering to the subject in need thereof at least one additional therapeutic agent.

In an embodiment, the one additional therapeutic agent is a taxane, a vinca alkaloid, a MEK inhibitor, an ERK inhibitor, topoisomerase inhibitor, or a RAF inhibitor.

According to an aspect of the invention, there is hereby provided a use of an conjugate or pharmaceutical composition of the invention, for the manufacture of a medicament for (i) treating a subject having or suspected of having a cancer, (ii) reducing or inhibiting the growth of a tumor in a subject, (iii) reducing or inhibiting a hematological cancer in a subject, or (iv) reducing or slowing the expansion of a cancer cell population in a subject.

According to an aspect of the invention, there is hereby provided an antibody-drug conjugate or pharmaceutical composition of the invention, for use in (i) treating a subject having or suspected of having a cancer, (ii) reducing or inhibiting the growth of a tumor in a subject, (iii) reducing or inhibiting a hematological cancer in a subject, or (iv) reducing or slowing the expansion of a cancer cell population in a subject.

Methods of producing the described ADC compounds and compositions are also disclosed. An exemplary embodiment is a method of producing an antibody-drug conjugate by reacting an antibody or antigen-binding fragment with a cleavable dual linker joined or covalently attached to a pharmaceutically active drug that is not a BH3 mimetic under conditions that allow conjugation.

The invention therefore provides the following numbered embodiments. It will be recognized that features specified in each embodiment may be combined with other specified features to provide further embodiments of the present invention.
Embodiment 1. A compound of Formula (A-1), or a pharmaceutically acceptable salt thereof:

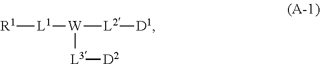

wherein:
R¹ is an attachment group;
L¹ is a bridging spacer;
W is a branching moiety;
L^{2′} and L³ are each independently a linker; and
D¹ and D² are each independently a pharmaceutically active drug, optionally with the proviso that neither D¹ nor D² is a BH3 mimetic.
Embodiment 2. The compound or pharmaceutically acceptable salt thereof according to Embodiment 1, wherein each of L^{2′} and L^{3′} comprises a cleavable group, optionally at least one cleavable group comprises a phosphate group, a pyrophosphate group, a peptide group, and/or a self-immolative group (e.g. a peptide group and a self-immolative group).
Embodiment 2a. The compound or pharmaceutically acceptable salt thereof according to Embodiment 2, wherein each of L^{2′} and L^{3′} comprises a peptide group and a self-immolative group.
Embodiment 2b. The compound or pharmaceutically acceptable salt thereof according to Embodiment 1, wherein each of L^{2′} and L^{3′} comprises a cleavable group, optionally at least one cleavable group comprises a phosphate group, a pyrophosphate group, a sugar (e.g. glucuronamide) group, a peptide group, and/or a self-immolative group (e.g. a peptide group and a self-immolative group).
Embodiment 3. The compound or pharmaceutically acceptable salt thereof according to Embodiment 1, Embodiment 2 or Embodiment 2a, of formula (B-1):

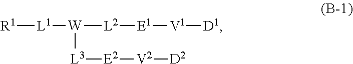

wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, a peptide group comprising 1 to 6 amino acids, wherein said peptide group is optionally substituted by a hydrophilic moiety;
V¹ and V² each independently comprise i) a self-immolative group, ii) an enzyme cleavage element (e.g. V¹ and V² are each independently a self-immolative group), or iii) a self-immolative group and an enzyme cleavage element; and
D¹ and D² are each independently a pharmaceutically active drug, optionally with the proviso that neither D¹ nor D² is a BH3 mimetic; or a pharmaceutically acceptable salt thereof.
Embodiment 3aa. The compound or pharmaceutically acceptable salt thereof according to Embodiment 3, wherein V¹ and V² are each independently i) a self-immolative group or ii) an enzyme cleavage element (e.g. V¹ and V² are each independently a self-immolative group).
Embodiment 3a. The compound or pharmaceutically acceptable salt thereof according to Embodiment 3 or Embodiment 3aa, wherein W is N or CH.
Embodiment 3b. The compound or pharmaceutically acceptable salt thereof according to Embodiment 3, Embodiment 3aa or Embodiment 3a, wherein E¹ and E² are each an enzyme cleavage element.
Embodiment 3c. The compound or pharmaceutically acceptable salt thereof according to Embodiment 3, Embodiment 3aa, Embodiment 3a or Embodiment 3b, wherein V¹ and V² are each independently a self-immolative group.
Embodiment 4. The compound or pharmaceutically acceptable salt thereof according to Embodiment 3, Embodiment 3aa, Embodiment 3a, Embodiment 3b or Embodiment 3c, wherein (i) the cleavable linker comprises a phosphate group, a pyrophosphate group and/or a self-immolative group; (ii) the cleavable linker comprises a self-immolative group; or (iii) the cleavable linker comprises a self-immolative group comprising —O—CH₂—, —NH—CH₂—, —OC(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium; or (iv) V¹ and/or V² each independently comprise a group comprising para-aminobenzyl-phosphate or para-aminobenzyl-pyrophosphate.
Embodiment 4a. The compound or pharmaceutically acceptable salt thereof according to Embodiment 4, wherein (i) the cleavable linker comprises a phosphate group, a pyrophosphate group and/or a self-immolative group; (ii) the cleavable linker comprises a self-immolative group; or (iii) the cleavable linker comprises a self-immolative group comprising —O—CH₂—, —NH—CH₂—, —OC(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.
Embodiment 5. The compound or pharmaceutically acceptable salt thereof according to Embodiment 3, Embodiment 4 or Embodiment 4a, of formula (C-1):

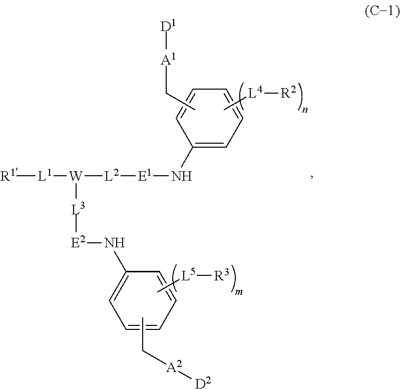

wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently a peptide group comprising 1 to 6 amino acids, wherein said peptide group is optionally substituted by a hydrophilic group;
A¹ and A² are each independently a bond, —OC(═O)—*,

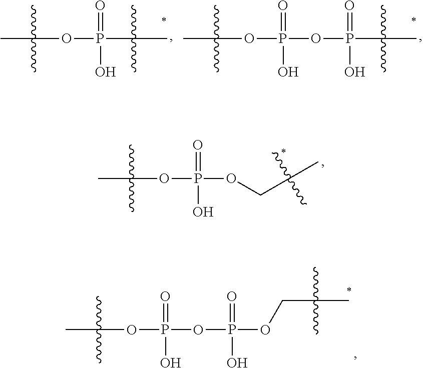 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆alkyl, and C₃-C₈cycloalkyl and the * of A¹ or A² indicates the point of attachment to D¹ or D²;
D¹ and D² are each independently a pharmaceutically active drug, optionally with the proviso that neither D¹ nor D² is a BH3 mimetic;
L⁴ and L⁵ are each independently a spacer moiety;
R² and R³ are each independently a hydrophilic group or an enzyme cleavage element (e.g. R² and R³ are each a hydrophilic group); and
m and n are each independently 0 or 1;
or a pharmaceutically acceptable salt thereof.
Embodiment 5a. The compound or pharmaceutically acceptable salt thereof according to Embodiment 5, wherein W is N or CH.
Embodiment 5b. The compound or pharmaceutically acceptable salt thereof according to Embodiment 5 or Embodiment 5a, wherein R² and R³ are each a hydrophilic group.
Embodiment 6. The compound or pharmaceutically acceptable salt thereof according to Embodiment 5, Embodiment 5a, or Embodiment 5b, of formula (D1-1), (D2-1) or (D3-1):

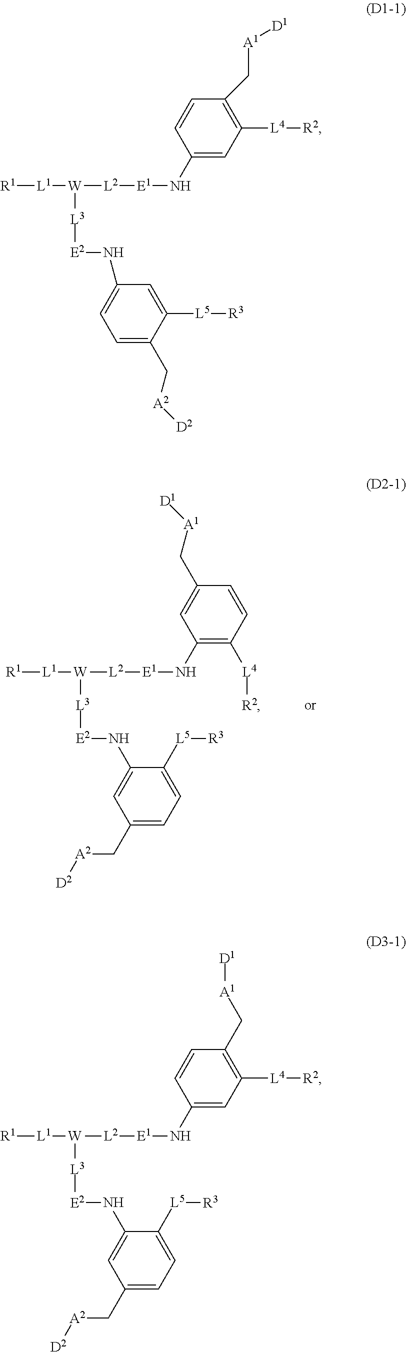

or a pharmaceutically acceptable salt thereof.
Embodiment 7. The compound or pharmaceutically acceptable salt thereof according to Embodiment 6, wherein for Formula (D1-1), R² and R³ are each independently a hydrophilic group, wherein for Formula (D2-1), R² and R³ are each independently an enzyme cleavage element; and for Formula (D3-1), R² is a hydrophilic group and R³ is an enzyme cleavage element;
or a pharmaceutically acceptable salt thereof.
Embodiment 8. The compound or pharmaceutically acceptable salt thereof according to any one of the preceding Embodiments, wherein R¹ is selected from the group consisting of:

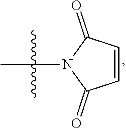 [SMILES: *N1C(=O)C=CC1=O]

—ONH₂, —NH₂,

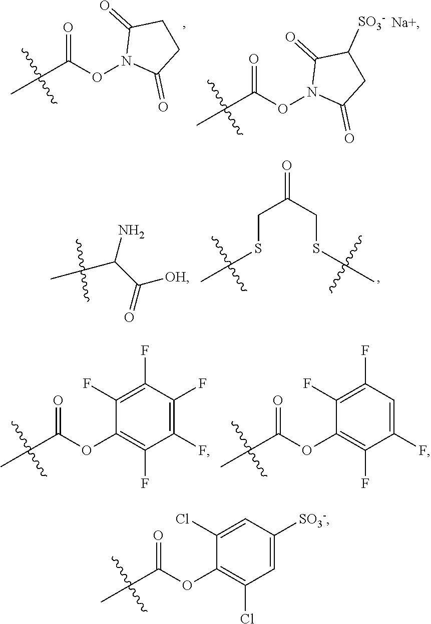

—N₃,

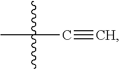 [SMILES: *C#C]

—SH, —SR¹¹, —SSR¹², —S(═O)₂(CH═CH₂), —(CH₂)₂S(═O)₂(CH═CH₂), —NHS(═O)₂(CH═CH₂), —NR¹¹C(═O)CH₂Br e.g. —NHC(═O)CH₂Br, —NR¹¹C(═O)CH₂I, e.g. —NHC(═O)CH₂I,

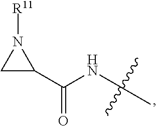

—C(O)NHNH₂,

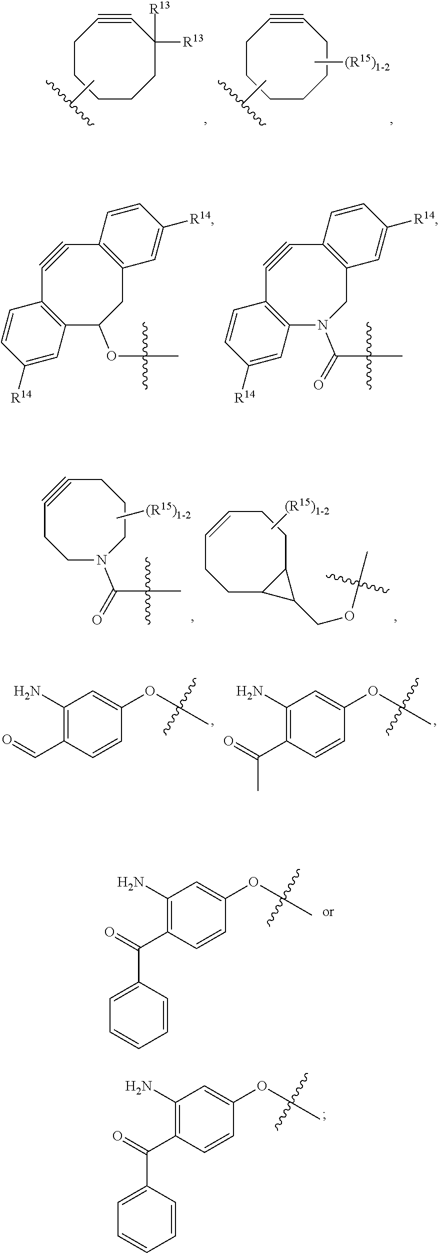

wherein:
each R¹¹ is independently selected from H and C₁-C₆alkyl;
each R¹² is 2-pyridyl or 4-pyridyl;
each R¹³ is independently selected from H, C₁-C₆alkyl, F, Cl, and —OH;
each R¹⁴ is independently selected from H, C₁-C₆alkyl, F, Cl, —NH₂, —OCH₃, —OCH₂CH₃, —N(CH₃)₂, —CN, —NO₂ and —OH; and
each R¹⁵ is independently selected from H, C₁-C₆alkyl, fluoro, benzyloxy substituted with —C(═O)OH, benzyl substituted with —C(═O)OH, C₁-C₄alkoxy substituted with —C(═O)OH and C₁-C₄alkyl substituted with —C(═O)OH.
Embodiment 9. The compound or pharmaceutically acceptable salt thereof according to any one of the preceding Embodiments, wherein R¹ is selected from the group consisting of:

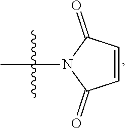 [SMILES: *N1C(=O)C=CC1=O]

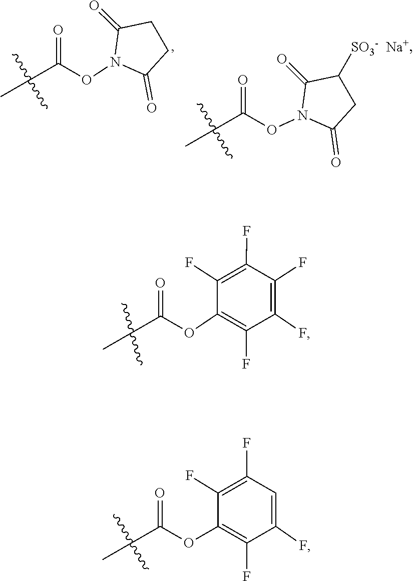

NHC(═O)CH₂Br and —NHC(═O)CH₂I.
Embodiment 9a. The compound or pharmaceutically acceptable salt thereof according to any one of the preceding Embodiments, wherein R¹ is selected from the group consisting of:

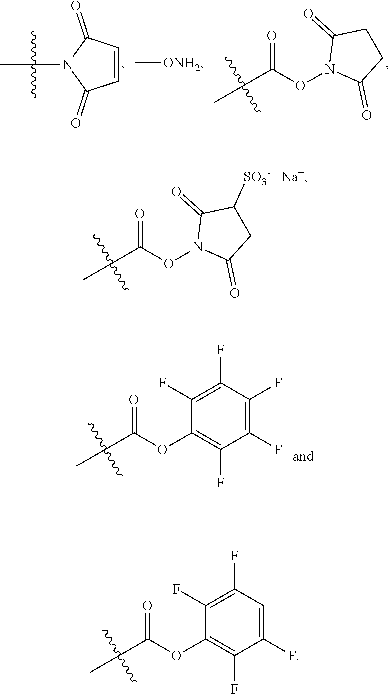

Embodiment 10. The compound or pharmaceutically acceptable salt thereof according to any one of the preceding Embodiments, wherein R¹ is

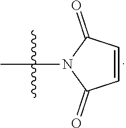 [SMILES: *N1C(=O)C=CC1=O]

Embodiment 11. The compound or pharmaceutically acceptable salt thereof according to any one of the preceding Embodiments, wherein:
   (1) L¹ comprises:

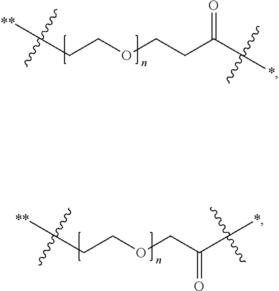

or *—CH(OH)CH(OH)CH(OH)CH(OH)—**,
wherein each n is an integer from 1 to 12, wherein the * of L¹ indicates the point of direct or indirect (e.g. direct) attachment to W, and the ** of L¹ indicates the point of direct or indirect (e.g. direct) attachment to R¹;
(2) L¹ is

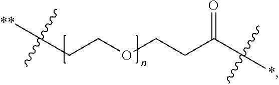

and n is an integer from 1 to 12 or n is 1 or n is 12, wherein the * of L¹ indicates the point of direct or indirect (e.g. direct) attachment to W, and the ** of L¹ indicates the point of direct or indirect (e.g. direct) attachment to R¹;
(3) L¹ is

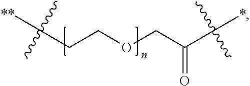

and n is an integer from 1 to 12, wherein the * of L¹ indicates the point of direct or indirect (e.g. direct) attachment to W, and the ** of L₁ indicates the point of direct or indirect (e.g. direct) attachment to R¹;
(4) L¹ comprises

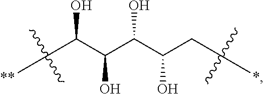 [SMILES: *C[C@H](O)[C@@H](O)[C@@H](O)[C@@H](*)O]

wherein the * of L¹ indicates the point of direct or indirect (e.g. direct) attachment to W, and the ** of L¹ indicates the point of direct or indirect (e.g. direct) attachment to R¹; or
(5) L¹ is a bridging spacer comprising:
*—C(═O)(CH₂)ₘO(CH₂)ₘ—**; *—C(═O)((CH₂)ₘO)ₜ(CH₂)ₙ—**; *—C(═O)(CH₂)ₘ—**;
*—C(═O)NH((CH₂)ₘO)ₜ(CH₂)ₙ—**;
*—C(═O)O(CH₂)ₘSSC(R^{L1})₂(CH₂)ₘC(═O)NR^{L1} (CH₂)ₘNR^{L1}C(═O)(CH₂)ₘ—**;
*—C(═O)O(CH₂)ₘC(═O)NH(CH₂)ₘ—**; *—C(═O)(CH₂)ₘNH(CH₂)ₘ—**;
*—C(═O)(CH₂)ₘNH(CH₂)ₙC(═O)—**; *—C(═O)(CH₂)ₘX¹(CH₂)ₘ—**;
*—C(═O)((CH₂)ₘO)ₜ(CH₂)ₙX₁(CH₂)ₙ—**; *—C(═O)(CH₂)ₘNHC(═O)(CH₂)ₙ—**;
*—C(═O)((CH₂)ₘO)ₜ(CH₂)ₙNHC(═O)(CH₂)ₙ—**;
*—C(═O)(CH₂)ₘNHC(═O)(CH₂)ₙX₁(CH₂)ₙ—**;
*—C(═O)((CH₂)ₘO)ₜ(CH₂)ₙNHC(═O)(CH₂)ₙX₁(CH₂)ₙ—**;
*—C(═O)((CH₂)ₘO)ₜ(CH₂)ₙC(═O)NH(CH₂)ₘ—**; *—C(═O)(CH₂)ₘC(R^{L1})₂—** or
*—C(═O)(CH₂)ₘC(═O)NH(CH₂)ₘ—**, wherein the * of L¹ indicates the point of direct or indirect (e.g. direct) attachment to W, and the ** of L¹ indicates the point of direct or indirect (e.g. direct) attachment to R¹;
X₁ is

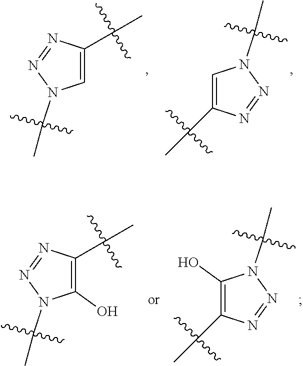 [SMILES: *c1cn(*)nn1 ; *c1cn(*)nn1 ; *c1nnn(*)c1O ; *c1nnn(*)c1O]

and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30; and
each R^{L1} is independently selected from H and C₁-C₆alkyl.
Embodiment 12. The compound or pharmaceutically acceptable salt thereof according to any one of the preceding Embodiments, wherein L¹ comprises a moiety represented by

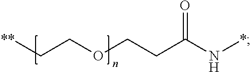

wherein n is an integer from 1 to 12 (e.g. 4 to 8, e.g. 4 or 8), wherein the * of L¹ indicates the point of direct or indirect (e.g. direct) attachment to W, and the ** of L¹ indicates the point of direct or indirect attachment to R¹.
Embodiment 12a. The compound or pharmaceutically acceptable salt thereof according to Embodiment 12, wherein n is an integer from 4 to 8.
Embodiment 12b. The compound or pharmaceutically acceptable salt thereof according to Embodiment 12a, wherein n is 4 or 8.
Embodiment 13. The compound or pharmaceutically acceptable salt thereof according to any one of the preceding Embodiments, wherein L¹ is represented by a formula

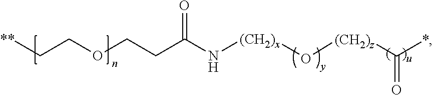

wherein
   n is an integer from 1 to 12 (e.g. 4 to 8, e.g. 4 or 8);
   x is an integer from 0 to 6 (e.g. 0 to 4, e.g. 0 to 2, e.g. 0 or 2);
   y is 0 or 1;
   z is an integer from 0 to 6 (e.g. 0 to 4, e.g. 0 to 2, e.g. 0 or 2);
   u is 0 or 1; and
   wherein the * of L¹ indicates the point of direct attachment to W, and the ** of L¹ indicates the point of direct attachment to R¹.
Embodiment 13a. The compound or pharmaceutically acceptable salt thereof according to Embodiment 13, wherein n is an integer from 4 to 8.
Embodiment 13b. The compound or pharmaceutically acceptable salt thereof according to Embodiment 13a, wherein n is 4 or 8.
Embodiment 13c. The compound or pharmaceutically acceptable salt thereof according to Embodiment 13, Embodiment 13a or Embodiment 13b, wherein x is an integer from 0 to 2.
Embodiment 13d. The compound or pharmaceutically acceptable salt thereof according to Embodiment 13c, wherein x is 0 or 2.
Embodiment 13e. The compound or pharmaceutically acceptable salt thereof according to Embodiment 13, Embodiment 13a, Embodiment 13b, Embodiment 13c or Embodiment 13d, wherein z is an integer from 0 to 2.
Embodiment 13f. The compound or pharmaceutically acceptable salt thereof according to Embodiment 13e, wherein z is 0 or 2.
Embodiment 14. The compound or pharmaceutically acceptable salt thereof according to any one of the preceding Embodiments, wherein L¹ is selected from the group consisting of:

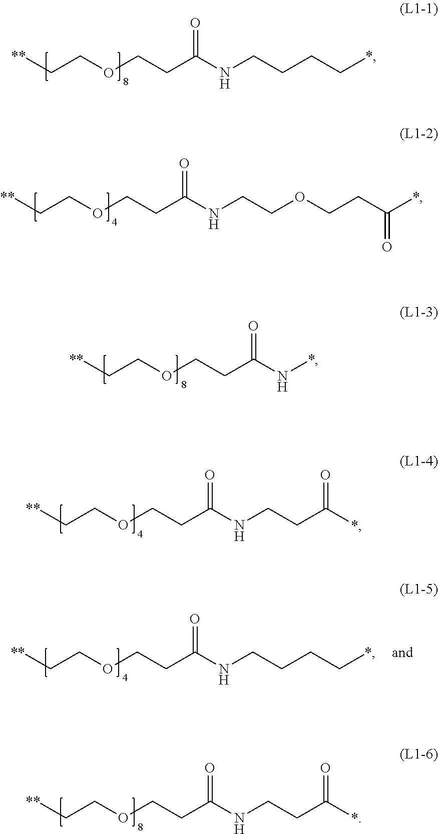

Embodiment 15. The compound or pharmaceutically acceptable salt thereof according to any one of Embodiments 3 to 14, wherein L² and L³ are each independently a connecting spacer comprising a moiety represented by:

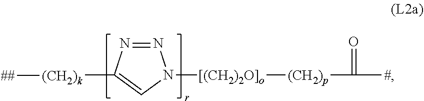

wherein
   k is an integer from 0 to 6;
   r is 0 or 1;
   is an integer from 0 to 12;
   p is an integer from 0 to 6; and
wherein the # of L² or L³ indicates the point of direct or indirect attachment to E¹ or E², respectively, and the ## of L² or L³ indicates the point of direct or indirect attachment to W.
Embodiment 16. The compound or pharmaceutically acceptable salt thereof according to any one of Embodiments 3 to 15, wherein
L² and L³ are each independently a connecting spacer selected from a group consisting of

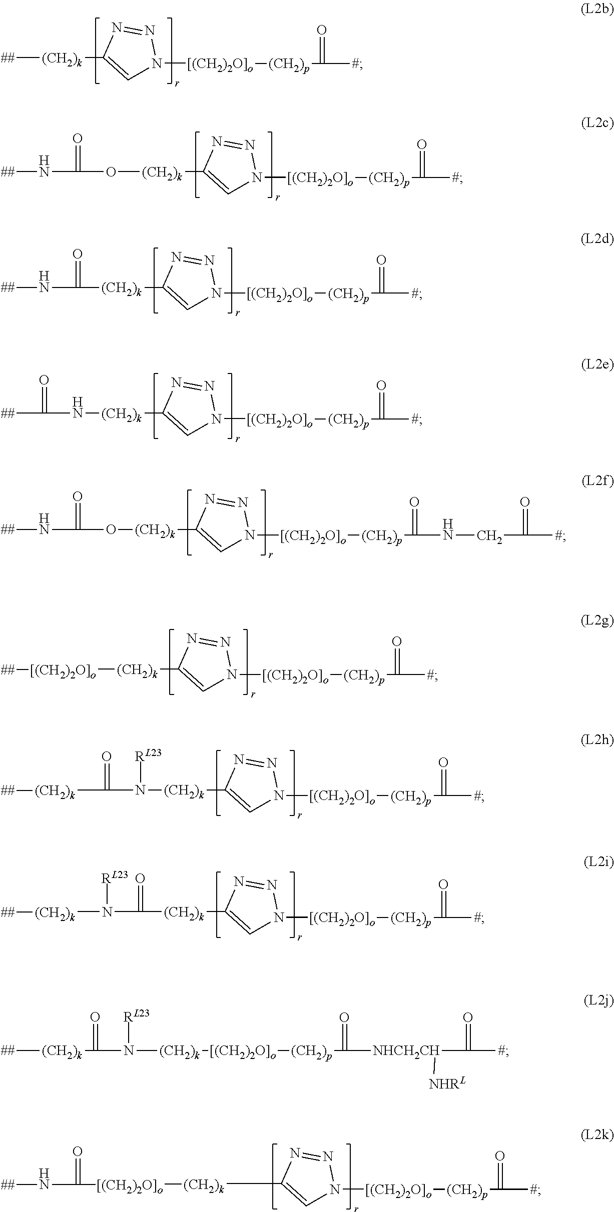

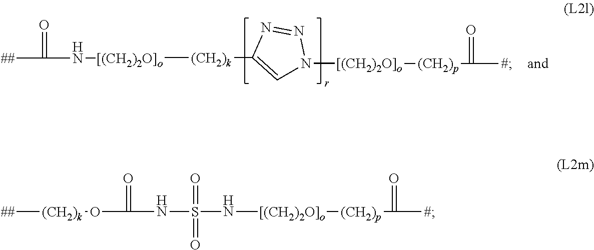

wherein
   k, in each occurrence, is independently an integer from 0 to 4;
   r, in each occurrence, is independently 0 or 1;
   o, in each occurrence, is independently an integer from 0 to 10;
   p, in each occurrence, is independently an integer from 0 to 4;
   R^{L23} is hydrogen or C₁-C₆alkyl;
   R^{L} is hydrogen or —C(O)—R^{H};
   R^{H} is a hydrophilic group; and
   the # of L² or L³ indicates the point of direct attachment to E¹ or E², respectively, and the
   ## of L² or L³ indicates the point of direct attachment to W;
   provided that when W is N, L² and L³ are not (L2c), (L2d), (L2f) or (L2k).
Embodiment 17. The compound or pharmaceutically acceptable salt thereof according to Embodiment 16, wherein L² and L³ are each independently a connecting spacer selected from a group consisting of:

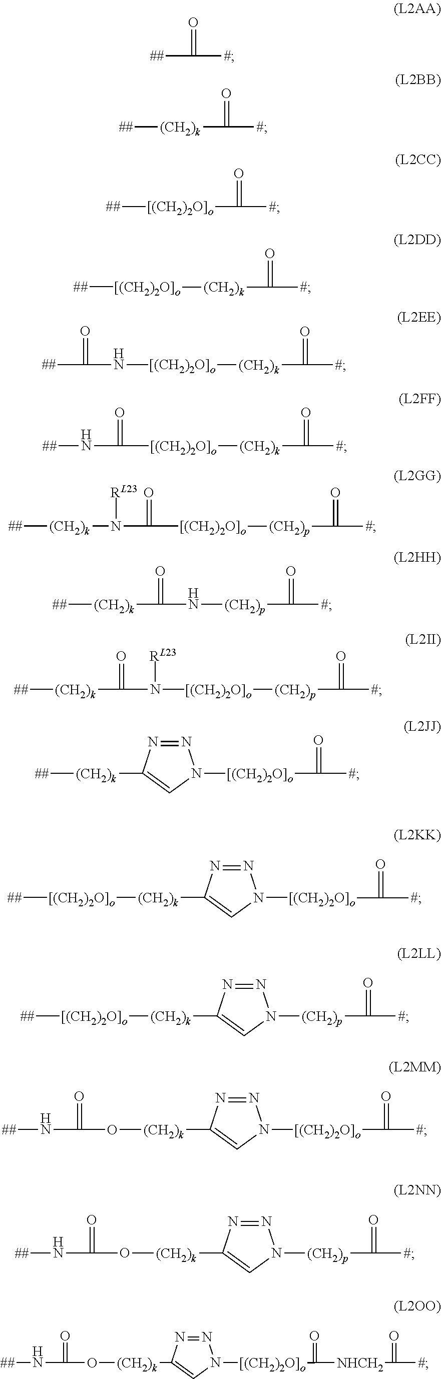

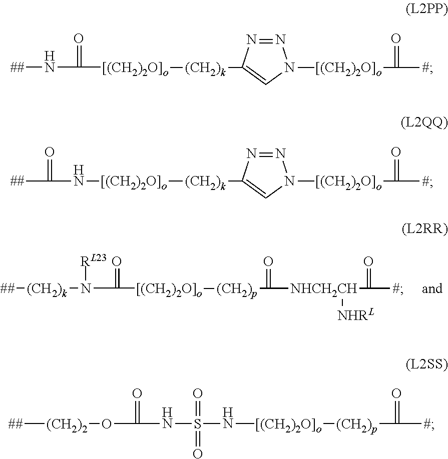

wherein
k, in each occurrence, is independently an integer from 1 to 3;
o, in each occurrence, is independently an integer from 1 to 9;
p, in each occurrence, is independently an integer from 1 to 3;
R^{L23} is hydrogen or C₁-C₃alkyl;
R^{L} is hydrogen or —C(O)—R^{H};
R^{H} is a hydrophilic group; and
the # of L² or L³ indicates the point of direct attachment to E¹ or E², respectively, and the ## of L² or L³ indicates the point of direct attachment to W; provided that when W is N, L² and L³ are not (L2FF), (L2MM), (L2NN), (L2OO), or (L2PP).
Embodiment 18. The compound or pharmaceutically acceptable salt thereof according to any one of Embodiments 3 to 17, wherein L² and L³, independently, are a connecting spacer selected from a group consisting of:

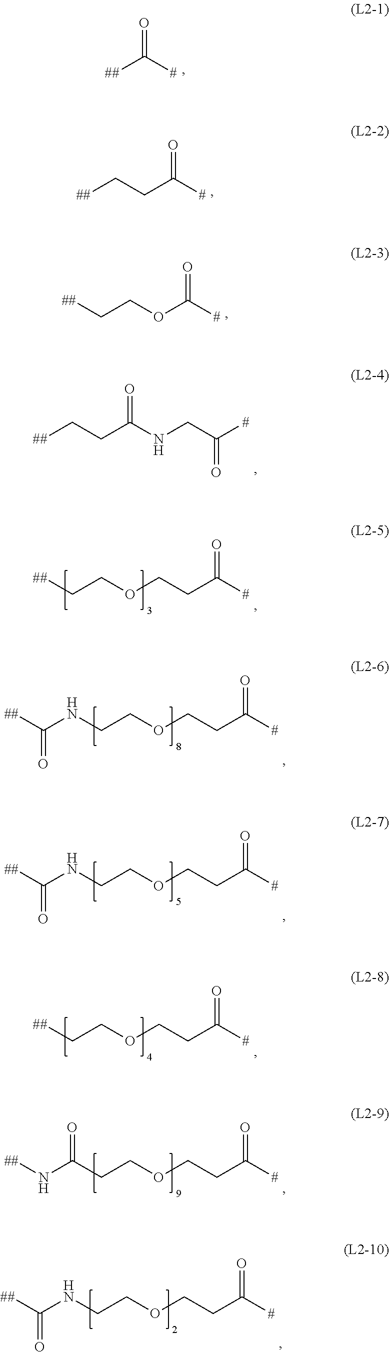

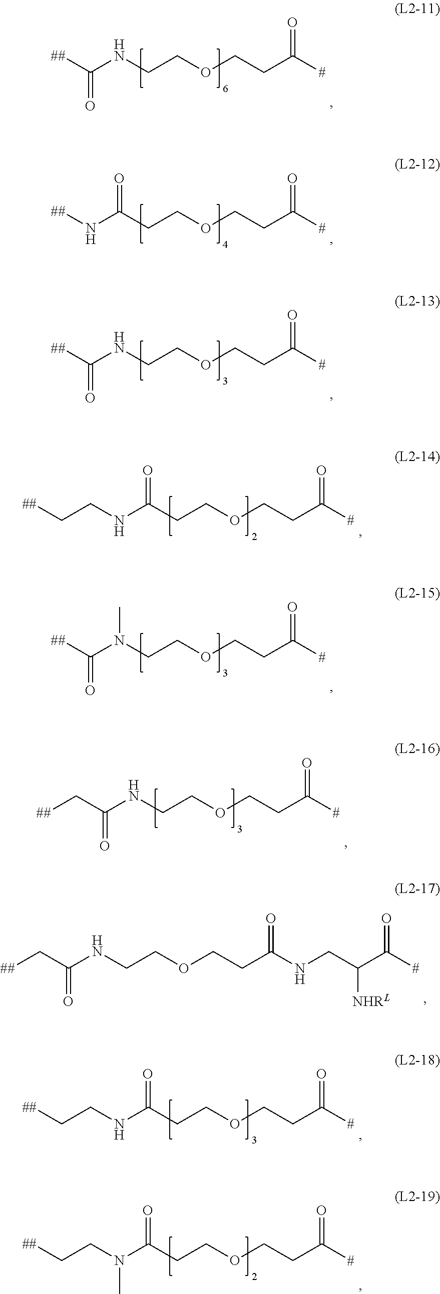

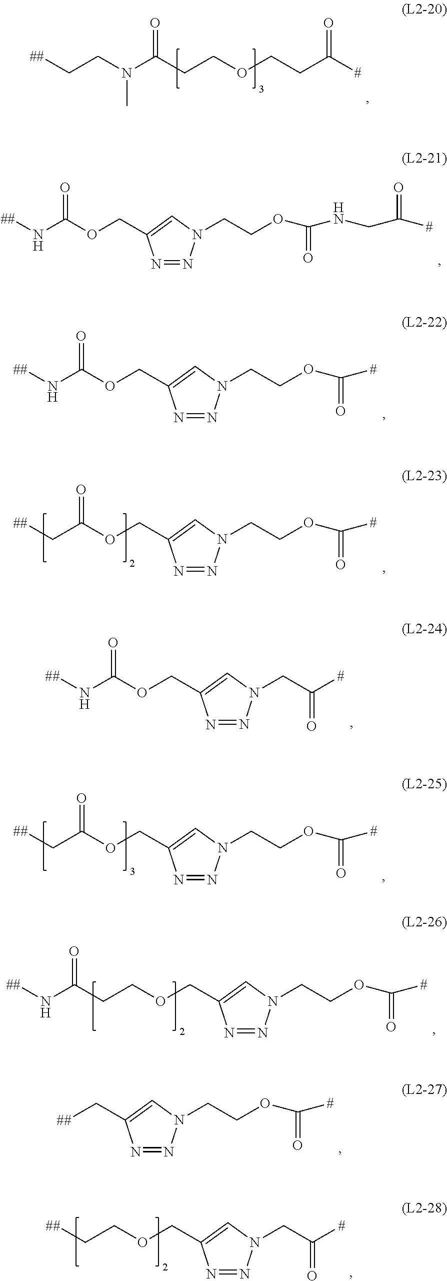

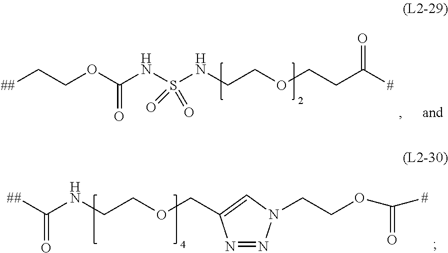

wherein the # of L² or L³ indicates the point of direct attachment to E¹ or E², respectively, the ## of L² or L³ indicates the point of direct attachment to W; R^{L} is hydrogen or —C(O)—R^{H}; and
R^{H} is

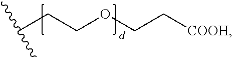

and d is an integer from 20 to 30 (e.g. 25).
Embodiment 18a. The compound or pharmaceutically acceptable salt thereof according to Embodiment 18, wherein d is 25.
Embodiment 19. The compound or pharmaceutically acceptable salt thereof according to any one of Embodiments 5 to 18a, wherein each peptide group independently comprises 1 to 4, 1 to 3, or 1 to 2 amino acid residues.
Embodiment 19a. The compound or pharmaceutically acceptable salt thereof according to Embodiment 19, wherein each peptide group independently comprises 2 amino acid residues.
Embodiment 20. The compound or pharmaceutically acceptable salt thereof according to Embodiment 19 or Embodiment 19a, wherein each amino acid residues is independently selected from glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), L-tyrosine (Tyr) and β-alanine (β-Ala).
Embodiment 21. The compound or pharmaceutically acceptable salt according to any one of the Embodiments 5 to 19 and 20, wherein each peptide group is independently selected from Val-Cit, Phe-Lys, Val-Ala, Val-Lys, Leu-Cit, Cit-(β-Ala), Gly-Gly-Gly, Gly-Gly-Phe-Gly, and sulfo-Ala-Val-Ala.
Embodiment 22. The compound or pharmaceutically acceptable salt according to any one of Embodiments 3 to 19a, wherein E¹ and/or E², independently, is/are each a peptide group selected from a group consisting of:

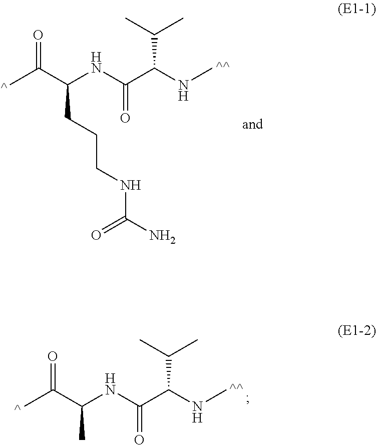

wherein \hat{ } of E1-1 or E1-2 indicates the point of direct attachment to V¹ or V² in Formula (B) or direct attachment to the —NH— group in Formula (C) and (D); and \hat{ }\hat{ } of E1-1 or E1-2 indicates the point of direct attachment to L² or L³, respectively (e.g. where E¹ and E² are each a peptide group independently selected from (E1-1) and (E1-2)).
Embodiment 22a. The compound or pharmaceutically acceptable salt thereof according to Embodiment 22, wherein E¹ and E² are each a peptide group independently selected from (E1-1) and (E1-2).
Embodiment 23. The compound or pharmaceutically acceptable salt thereof according to any one of Embodiments 3 to 19a, wherein E¹ and/or E², independently, is/are each a peptide group represented by

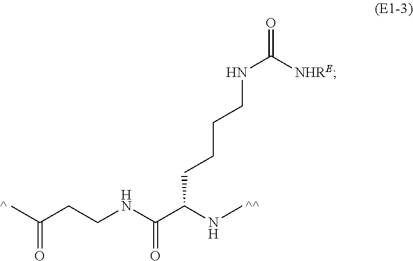

wherein R^{E} is a hydrophilic group R^{H}.
Embodiment 24. The compound or pharmaceutically acceptable salt thereof according to Embodiment 23, wherein the hydrophilic group R^{H} in (E1-3) is

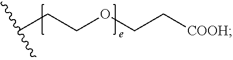

wherein e is an integer between 20 and 30 (e.g. 25).
Embodiment 24a. The compound or pharmaceutically acceptable salt thereof according to Embodiment 24, wherein e is 25.
Embodiment 25. The compound or pharmaceutically acceptable salt according to Embodiment 22, wherein E¹ and E² are each

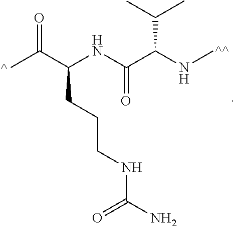 [SMILES: CN[C@H](C(=O)N[C@@H](CCCNC(N)=O)C(C)=O)C(C)C]

Embodiment 26. The compound or pharmaceutically acceptable salt according to any one of Embodiments 5 to 25, wherein A¹ and A² are independently selected from a bond and —OC(═O)—*, wherein * indicates the point of attachment to D¹ or D² respectively.
Embodiment 26a. The compound or pharmaceutically acceptable salt according to any one of Embodiments 5 to 25, wherein A¹ and A² are independently selected from a bond,

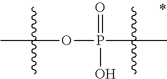 [SMILES: *OP(*)(=O)O]

and —OC(═O)—*, wherein * indicates the point of attachment to D¹ or D² respectively.
Embodiment 27. The compound or pharmaceutically acceptable salt according to Embodiment 26, wherein A¹ and A² are each a bond.
Embodiment 27a. The compound or pharmaceutically acceptable salt according to Embodiment 26, wherein A¹ and A² are both-OC(═O)—*.
Embodiment 27b. The compound or pharmaceutically acceptable salt according to Embodiment 26, wherein one of A¹ and A² is OC(═O)—*, and the other of A¹ and A² is a bond.
Embodiment 27c. The compound or pharmaceutically acceptable salt according to Embodiment 26a, wherein A¹ and A² independently are a bond or

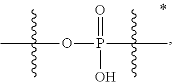 [SMILES: *OP(*)(=O)O]

wherein * indicates the point of attachment to D¹ or D².
Embodiment 27d. The compound or pharmaceutically acceptable salt according to Embodiment 27c, wherein one of A¹ and A² is

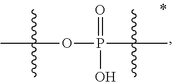 [SMILES: *OP(*)(=O)O]

and the other of A¹ and A² is a bond.
Embodiment 27e. The compound or pharmaceutically acceptable salt according to Embodiment 27c, wherein A¹ and A² are both

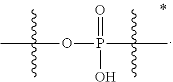 [SMILES: *OP(*)(=O)O]

Embodiment 27f. The compound or pharmaceutically acceptable salt according to Embodiment 26a, wherein one of A¹ and A² is

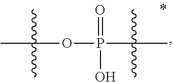 [SMILES: *OP(*)(=O)O]

and the other of A¹ and A² is OC(═O)—*.
Embodiment 27 g. The compound or pharmaceutically acceptable salt according to Embodiment 26a, wherein) A¹ and A² a-e-OC(═O)—*;
(ii) A¹ and A² are

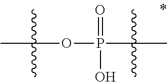 [SMILES: *OP(*)(=O)O]

(iii) A¹-s —OC(═O)—* and A² is a bond
(iv) A¹-s —OC(═O)—* and A² is

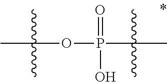 [SMILES: *OP(*)(=O)O]

(v) A¹ is a bond and A² is

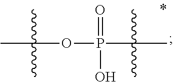 [SMILES: *OP(*)(=O)O]

or
(vi) A¹ is a bond and A²-s —OC(═O)—*,
wherein * indicates the point of attachment to D¹ or D².
Embodiment 28. The compound or pharmaceutically acceptable salt according to any one of Embodiments 5 to 27b, wherein:
   i) L⁴ and L⁵ are each independently a spacer moiety having the structure

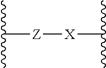

wherein:
Z is —O—, —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{L45}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is a bond, triazolyl, or —CH₂—triazolyl-,
wherein X is connected to R² or R³; or
(ii) L⁴ and L⁵, independently, are a spacer moiety having the structure

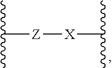

wherein:
Z is —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{b}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is —CH₂—triazolyl-C₁-C₄alkylene-OC(O)NHS(O)₂NH—, —C₄-C₆cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂—triazolyl-C₁-C₄alkylene-OC(O)NHS(O)₂NH—(CH₂CH₂O)ₙ—, —C₄-C₆cycloalkylene-OC(O)NHS(O)₂NH—(CH₂CH₂O)ₙ—, wherein each n independently is 1, 2, or 3,
wherein X is connected to R² or R³.
Embodiment 29. The compound or pharmaceutically acceptable salt according to Embodiment 28, wherein Z is —O—, —CH₂NR^{L45}C(═O)—, —CH₂NR^{L45}C(═O)NH— or —CH₂O—; X is a bond, triazolyl, or —CH₂—triazolyl-; and R^{L45}, in each occurrence, is independently H or C₁-C₃alkyl.
Embodiment 30. The compound or pharmaceutically acceptable salt according to any one of Embodiments 5 to 29, wherein L⁴ and L⁵ are each independently a spacer moiety selected from a group consisting of

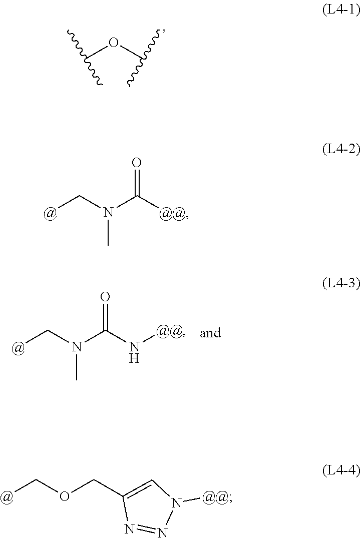

wherein the @ of L⁴ or L⁵ indicates the point of direct attachment to the phenyl group, and the @@ of L⁴ or L⁵ indicates the point of direct attachment to R² or R³.
Embodiment 31. The compound or pharmaceutically acceptable salt thereof according to any one of Embodiments 5 to 30, wherein the hydrophilic groups represented by R² and R³ each independently comprises polyethylene glycol, polyalkylene glycol, a polyol, a polysarcosine, a sugar, an oligosaccharide, a polypeptide, C₂-C₆alkyl substituted with 1 to 3

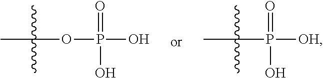 [SMILES: *OP(=O)(O)O ; *P(=O)(O)O]

or C₂-C₆alkyl substituted with 1 to 2 substituents independently selected from —OC(═O)NHS(O)₂NHCH₂CH₂OCH₃, —NHC(═O)C₁-C₄alkylene-P(O)(OCH₂CH₃)₂ and —COOH groups.
Embodiment 32. The compound or pharmaceutically acceptable salt thereof according to any one of Embodiments 5 to 30, wherein R² or/and R³ independently is/are each selected from the group consisting of:

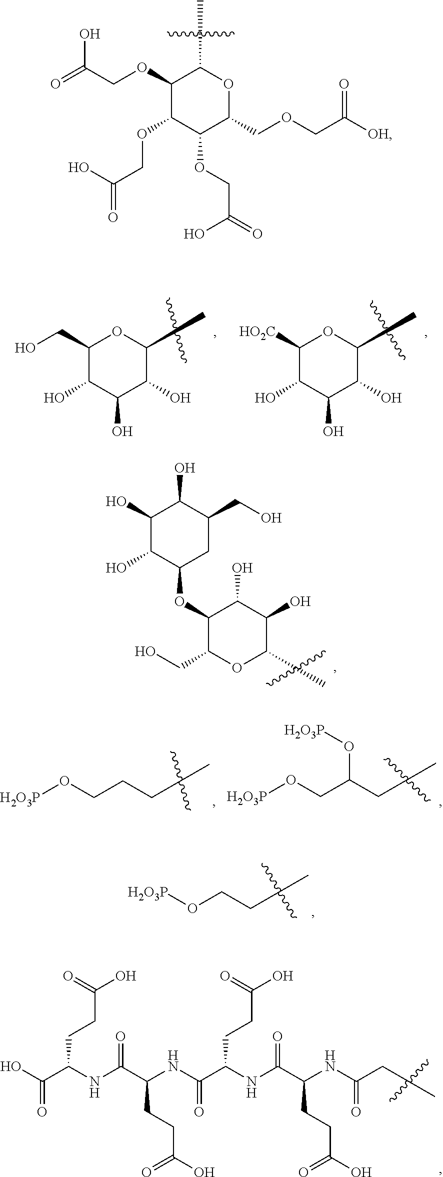

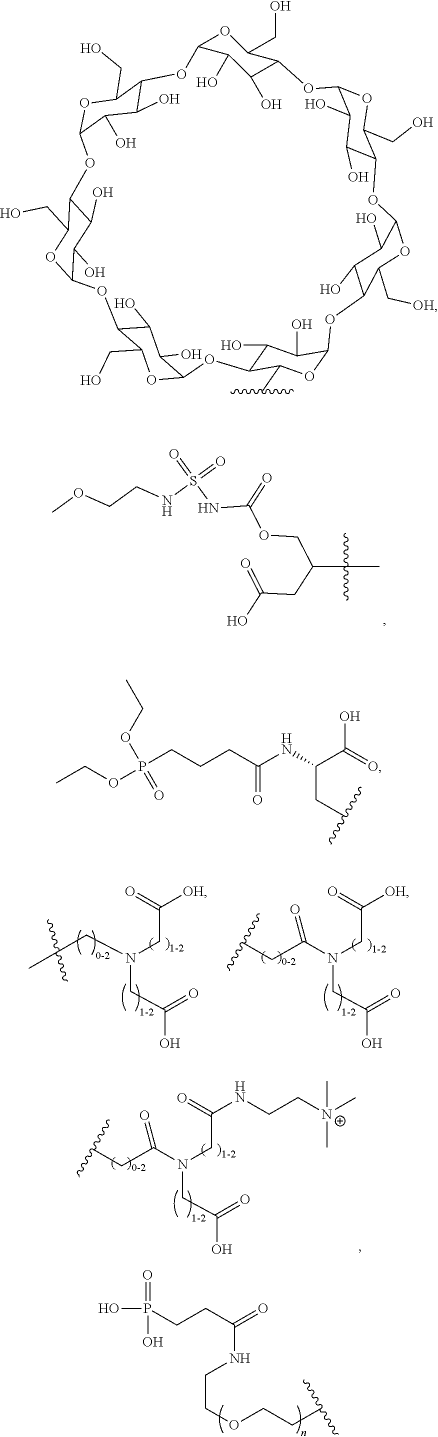

wherein
n is an integer between 1 and 6,

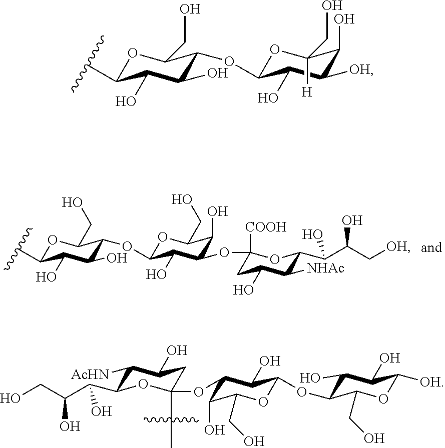

Embodiment 33. The compound or pharmaceutically acceptable salt according to any one of Embodiments 5 to 30, wherein R² or/and R³ independently comprises:
   (i) a polysarcosine with the following moiety:

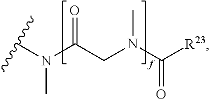

wherein
f is an integer between 3 and 25; and
R²³ is H, —CH₃ or —CH₂CH₂C(═O)OH;
(ii) a polyethylene glycol of formula:

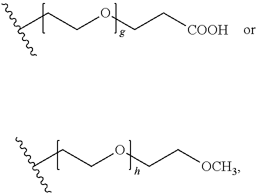

wherein
g and h are independently an integer between 2 and 30; or
(iii) a moiety represented by

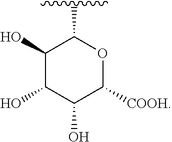 [SMILES: *[C@@H]1O[C@H](C(=O)O)[C@H](O)[C@H](O)[C@H]1O]

Embodiment 33a. The compound or pharmaceutically acceptable salt according to any one of Embodiments 5 to 30, wherein the hydrophilic group represented by R² or R³ each independently comprises:
(i) a polysarcosine with the following moiety:

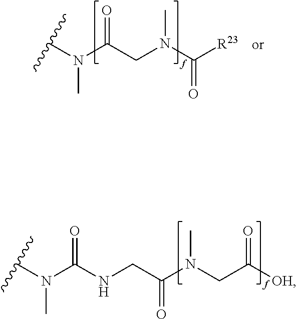

Wherein f is an integer between 3 and 25; and R²³ is H, —CH₃ or —CH₂CH₂C(═O)OH; or
(ii) a polyethylene glycol of formula:

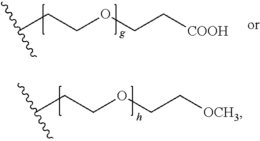

wherein g and h are independently an integer between 2 and 30; or
(iii) a moiety represented by

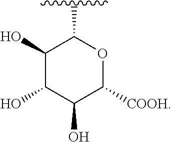 [SMILES: *[C@@H]1O[C@H](C(=O)O)[C@@H](O)[C@H](O)[C@H]1O]

Embodiment 34. The compound or pharmaceutically acceptable salt according to any one of Embodiments 5 to 30, wherein R² or/and R³, independently, is/are each selected from a group consisting of

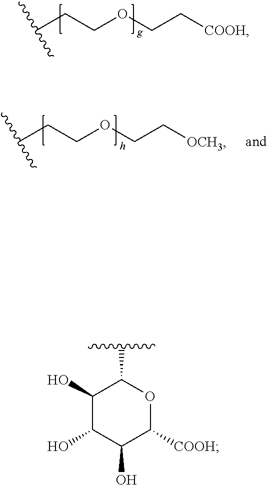

wherein g and h are independently an integer between 20 and 30 (e.g. 23 or 24).
Embodiment 35. The compound or pharmaceutically acceptable salt thereof according to Embodiment 34, wherein R² or/and R³ comprises:

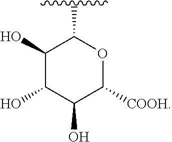 [SMILES: *[C@@H]1O[C@H](C(=O)O)[C@@H](O)[C@H](O)[C@H]1O]

Embodiment 35a. The compound or pharmaceutically acceptable salt thereof according to Embodiment 34, wherein R² and R³ each independently comprises a polyethylene glycol of formula:

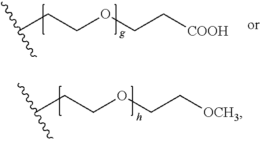

wherein g and h are independently an integer between 2 and 30.
Embodiment 36. The compound or pharmaceutically acceptable salt according to any one of the preceding Embodiments, wherein D¹ and D² are the same.
Embodiment 37. The compound or pharmaceutically acceptable salt according to any one of Embodiments 1 to 35a, wherein D¹ and D² are different.
Embodiment 38. The compound or pharmaceutically acceptable salt according to any one of the preceding Embodiments, wherein D¹ and D² are each independently selected from a cytotoxic drug, a cytostatic drug and an immunosuppressive drug.
Embodiment 39. The compound or pharmaceutically acceptable salt according to any one of the Embodiments 1 to 37, wherein D¹ and D² are each independently selected from an auristatin, a camptothecin, a duocarmycin, an etoposide, a maytansine, a maytansinoid, a taxane, a benzodiazepine or benzodiazepine containing drug (e.g., pyrrolo[1,4]-benzodiazepines (PBDs), indolinobenzodiazepines, and oxazolidinobenzodiazepines), and a vinca alkaloid.
Embodiment 39a. The compound or pharmaceutically acceptable salt according to any one of Embodiments 1 to 37, wherein D¹ and D² are each independently selected from the group consisting of:

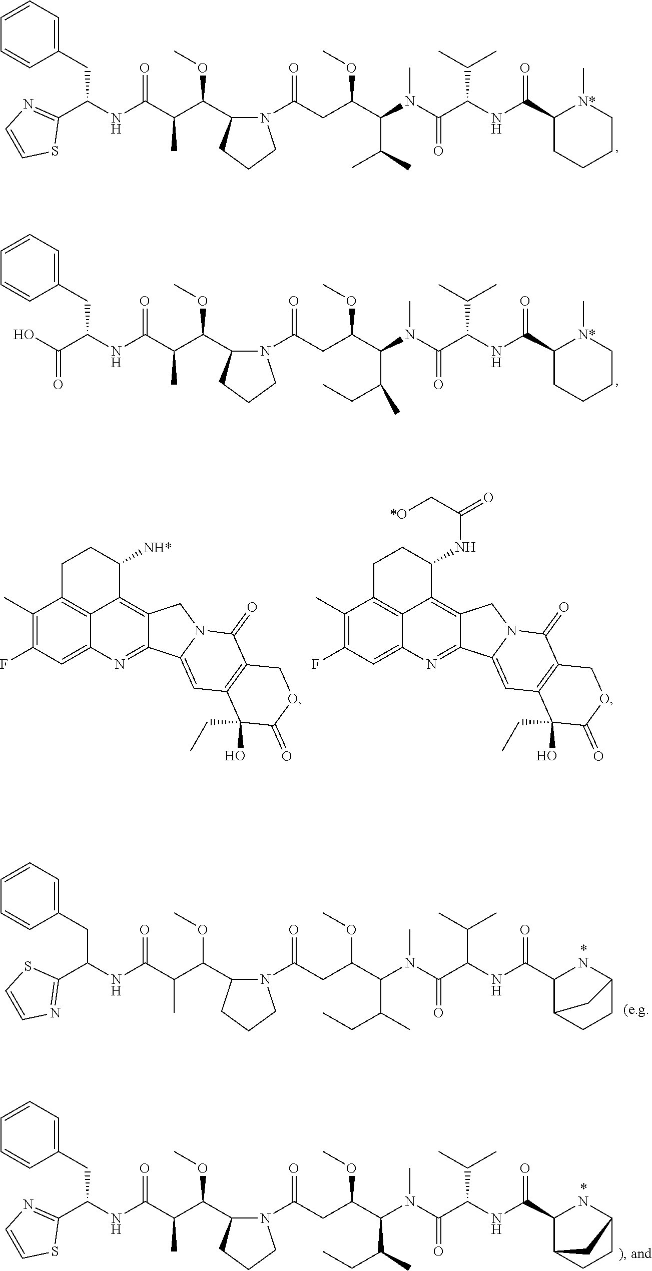

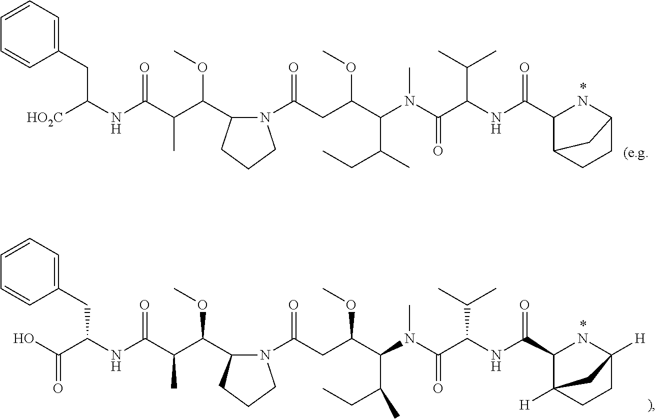

wherein * indicates the point of attachment to A¹ or A²; or a pharmaceutically acceptable salt thereof.
Embodiment 39b. The compound or pharmaceutically acceptable salt according to Embodiment 39a, wherein:

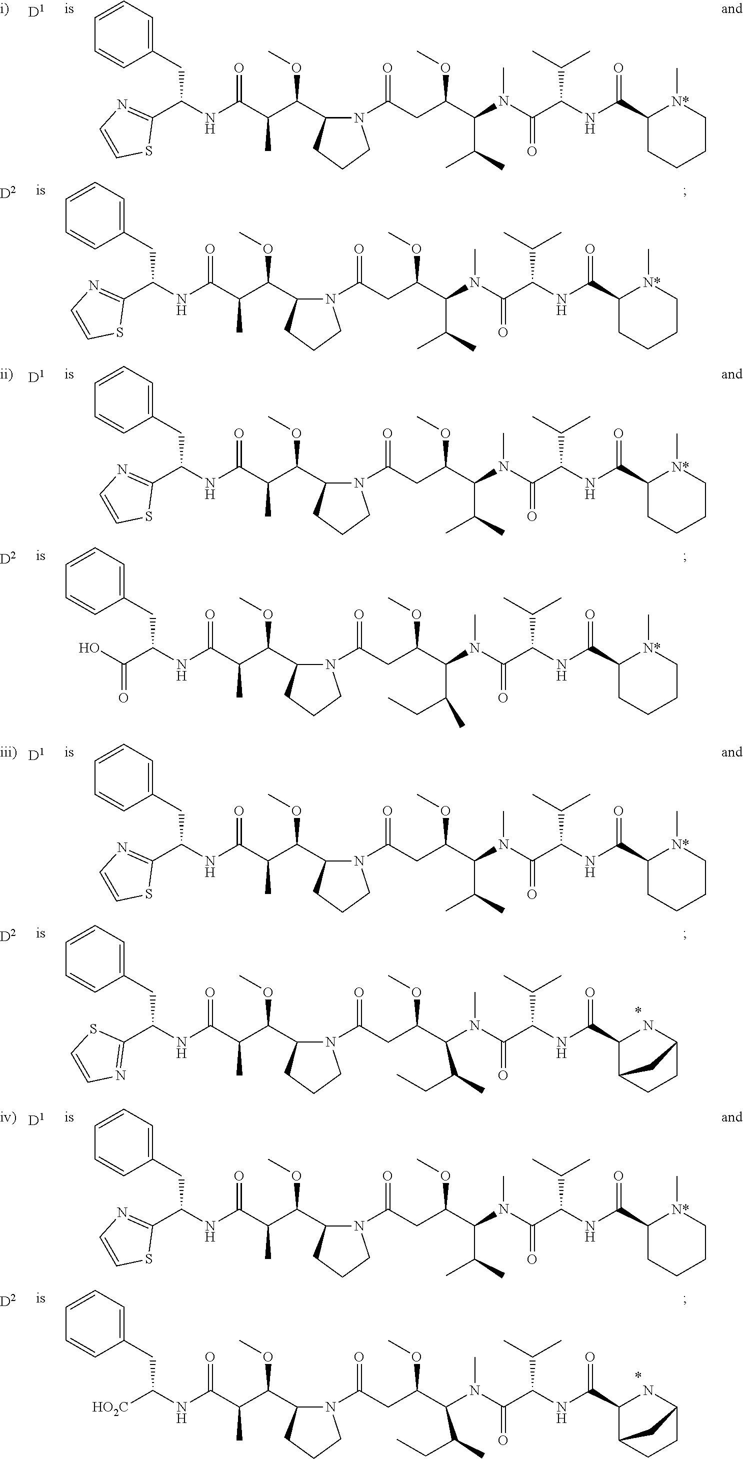

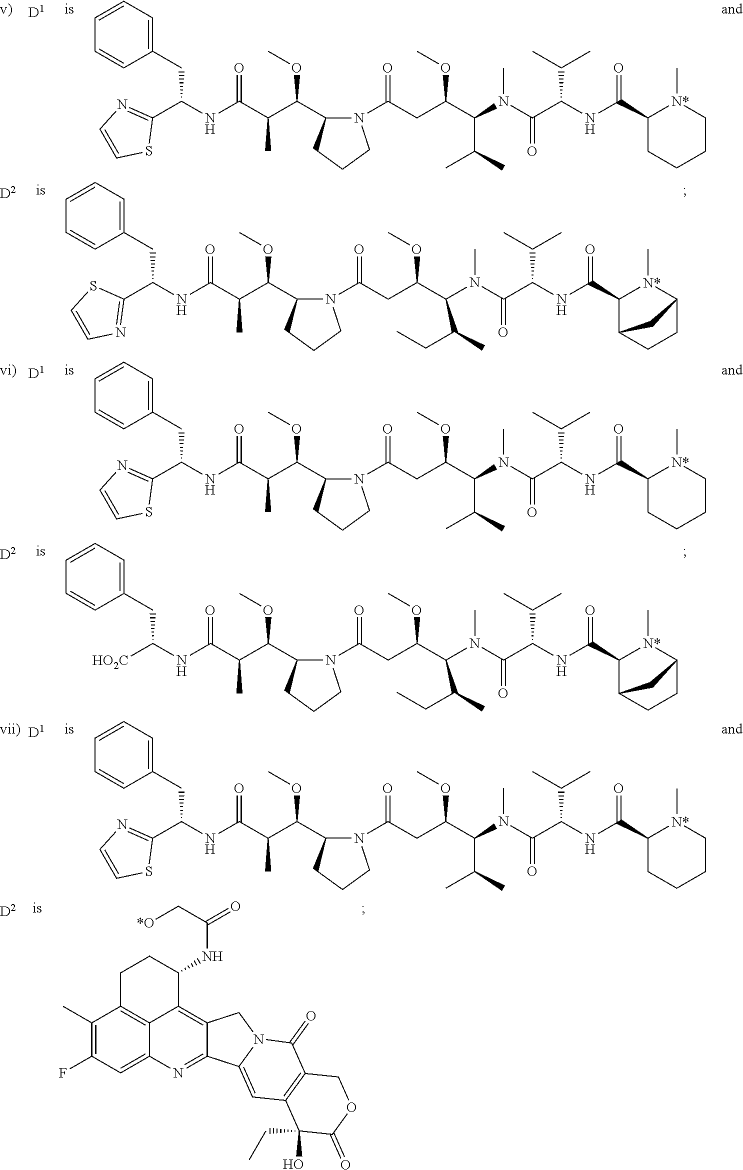

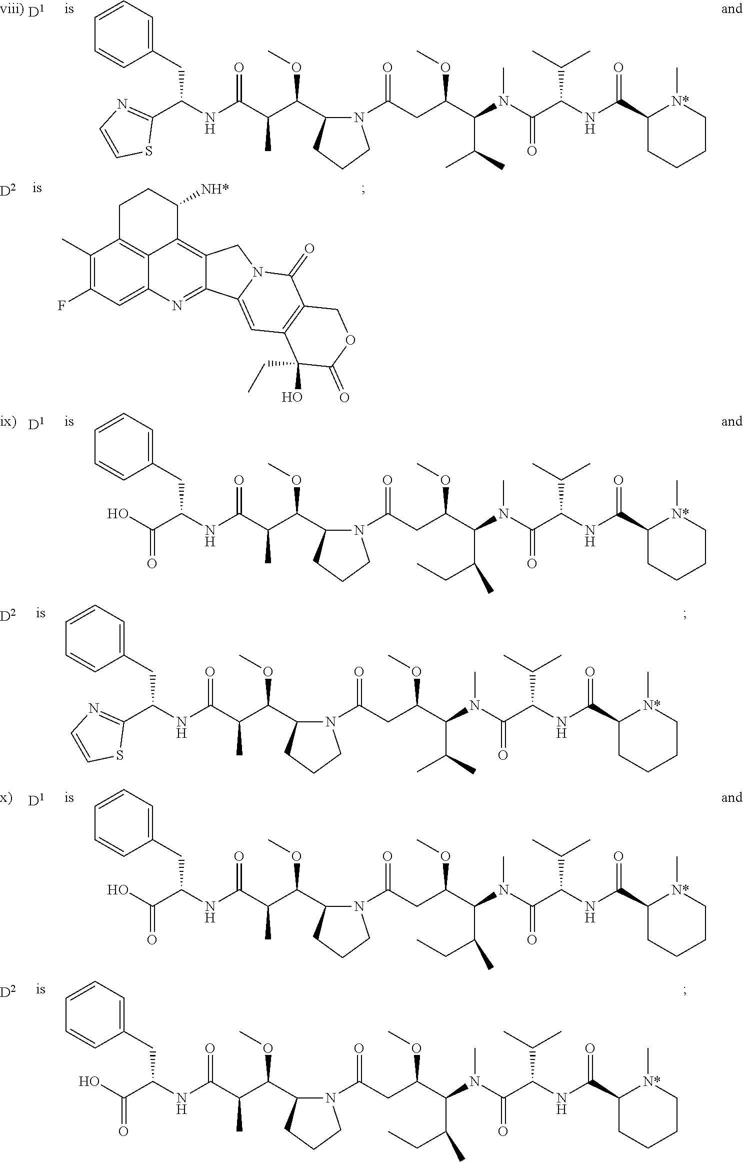

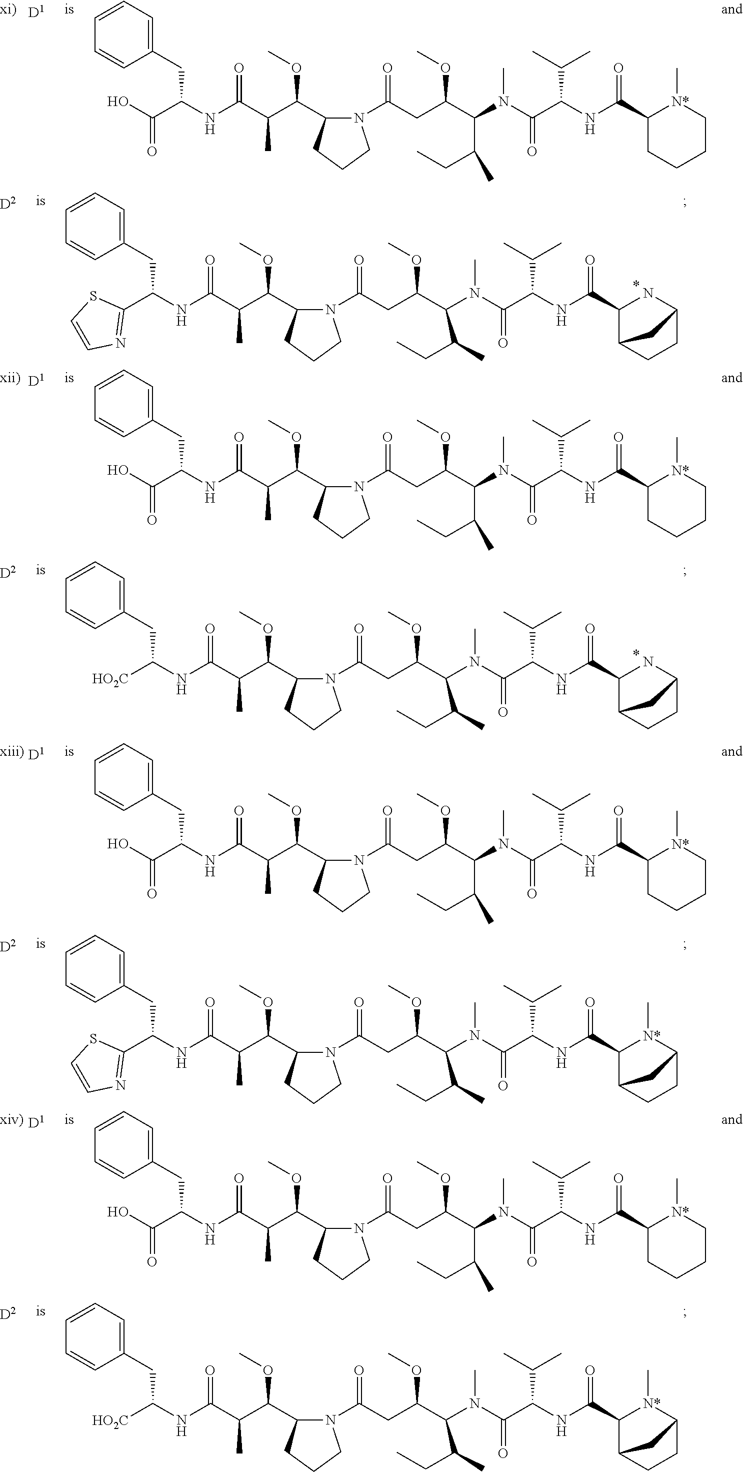

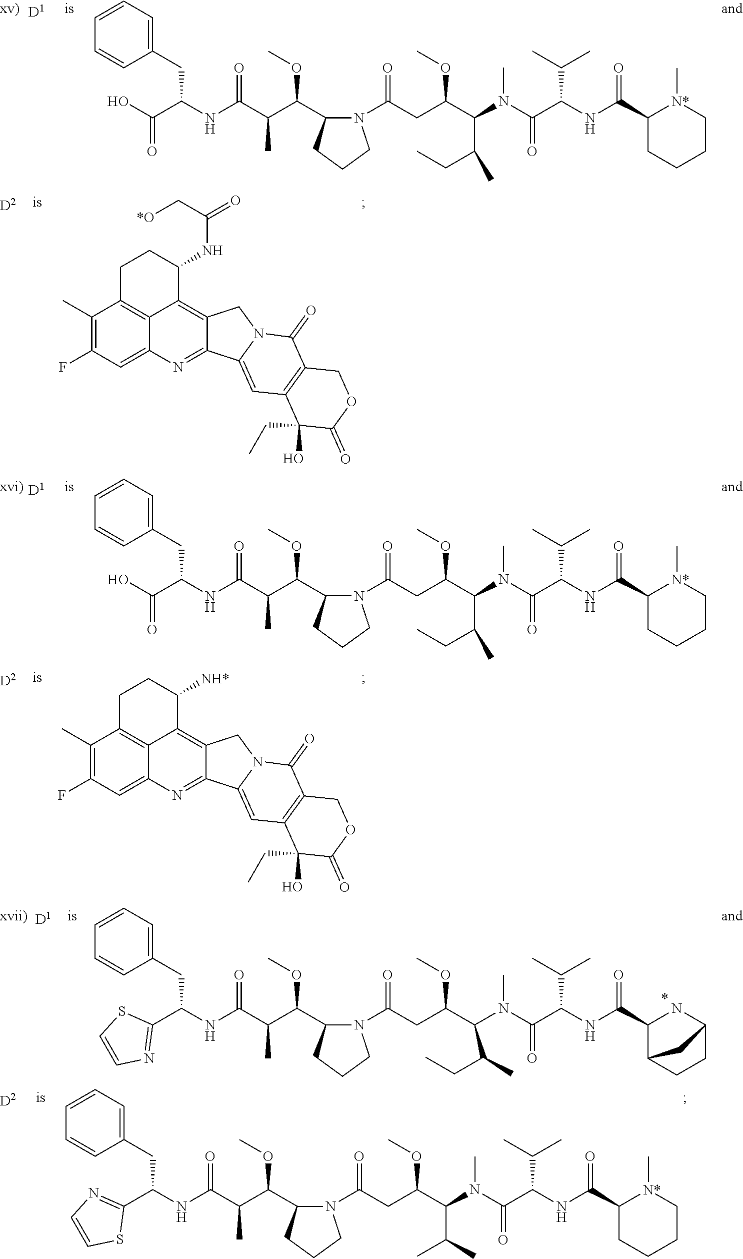

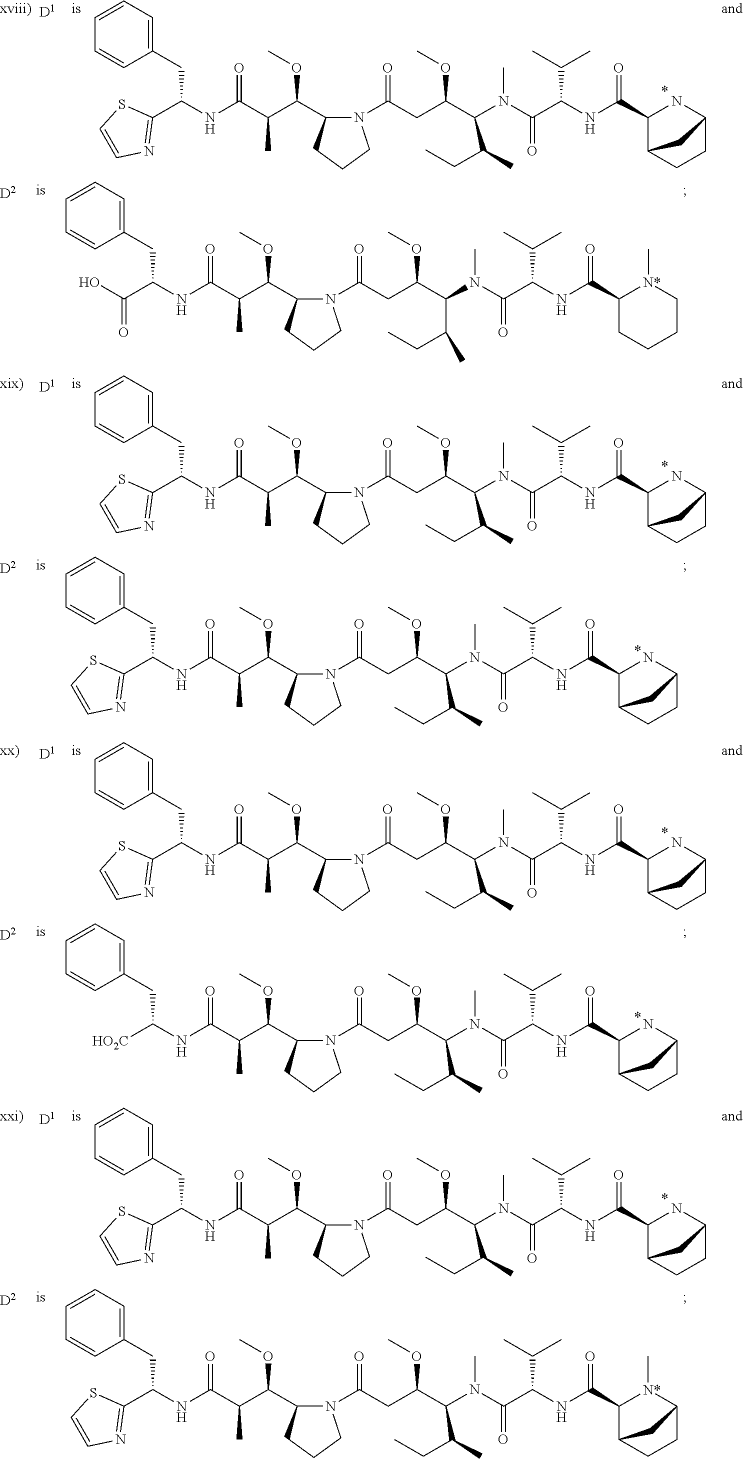

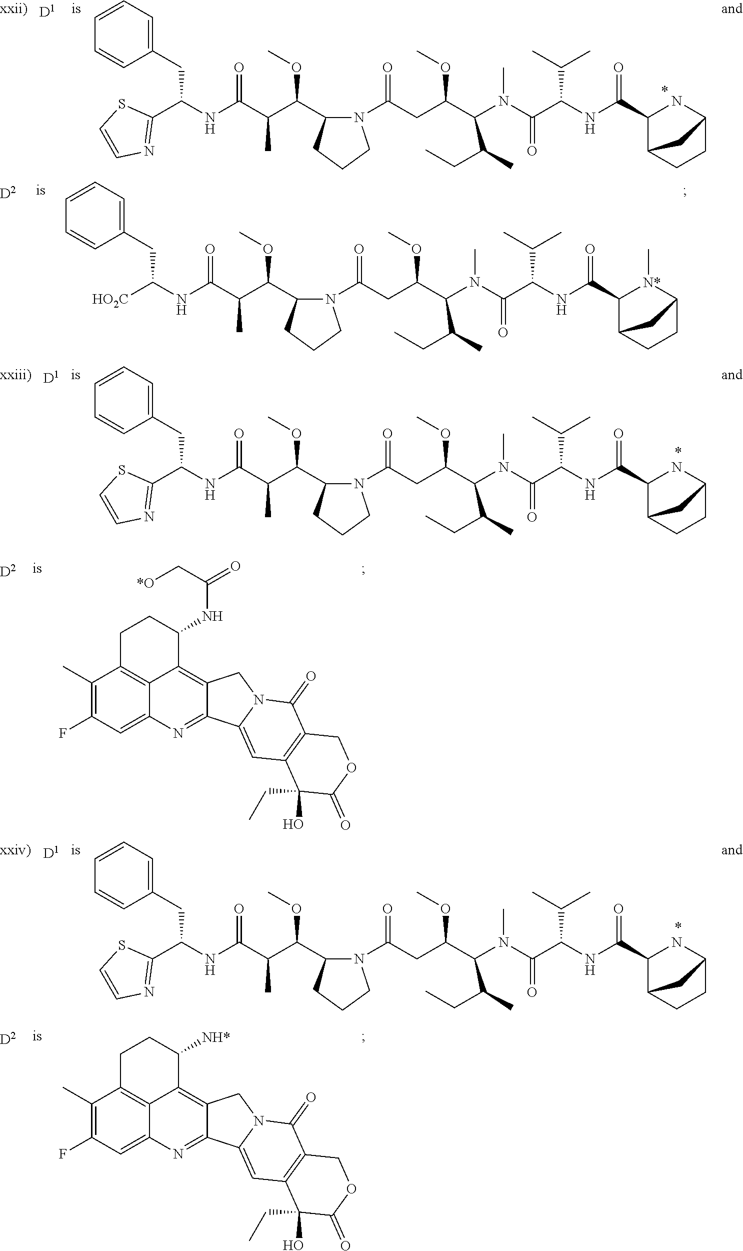

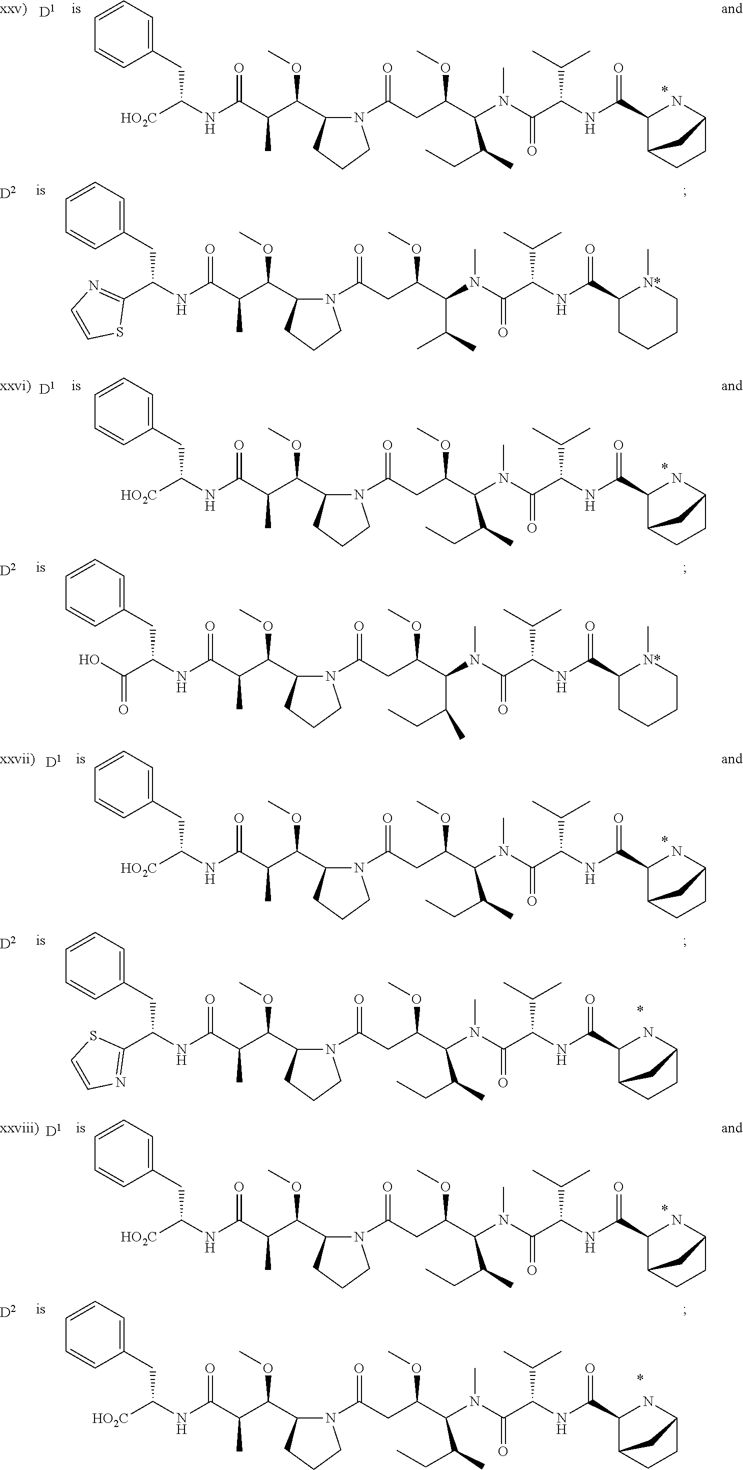

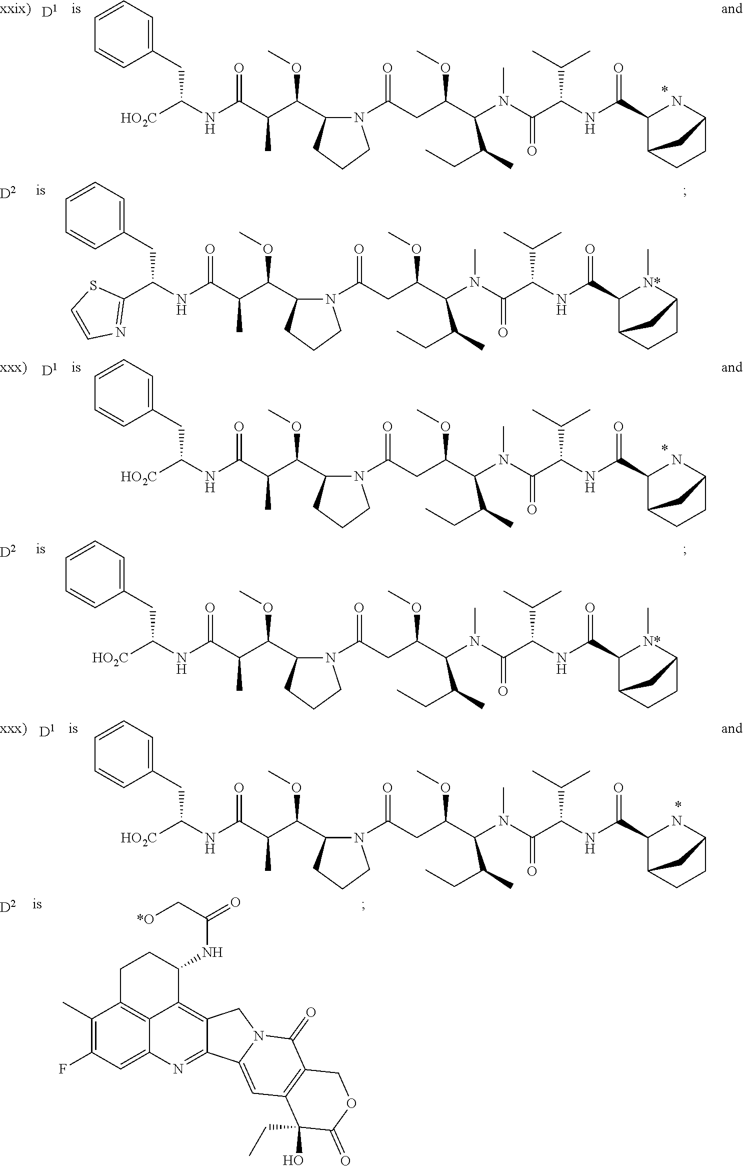

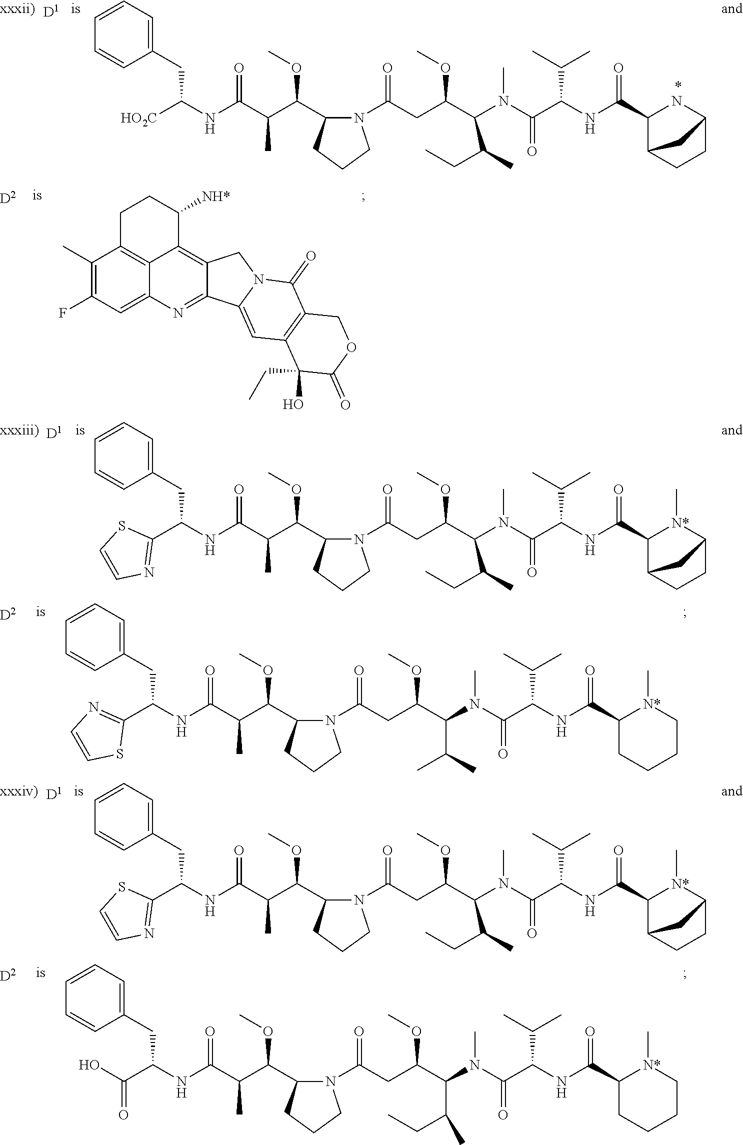

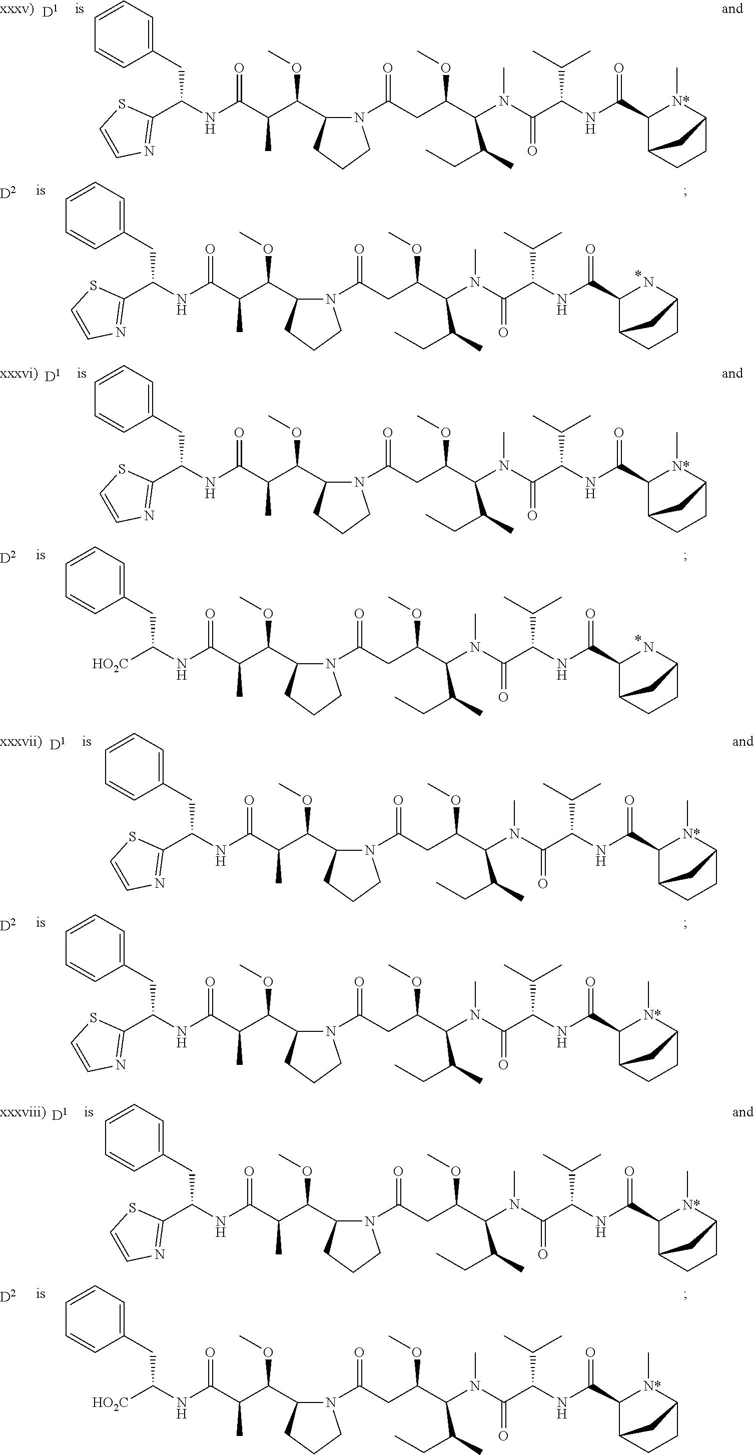

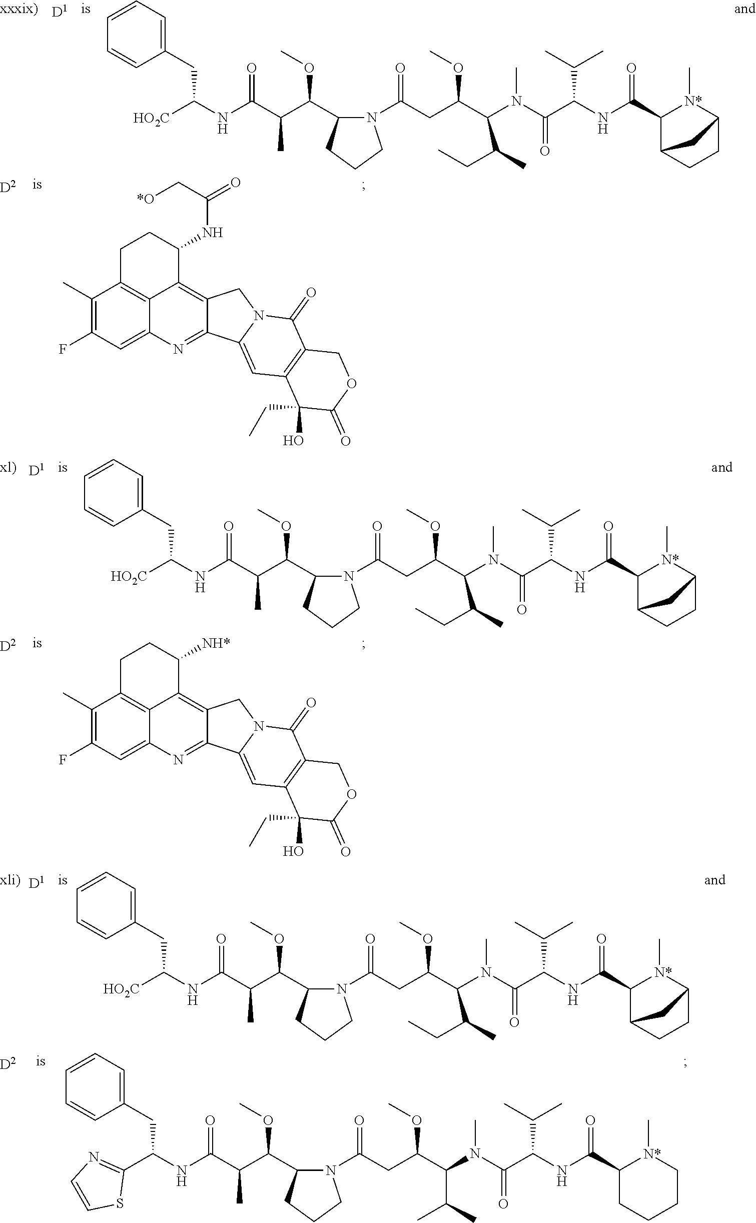

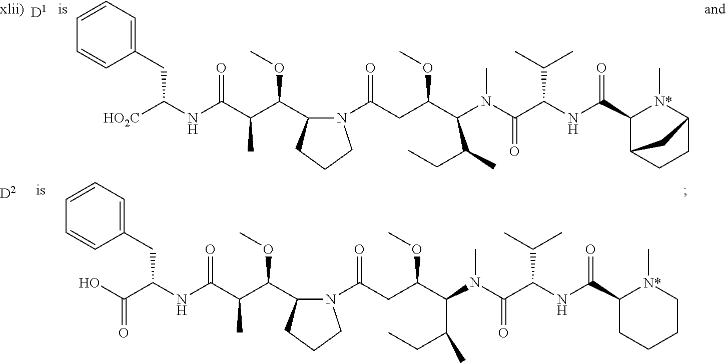

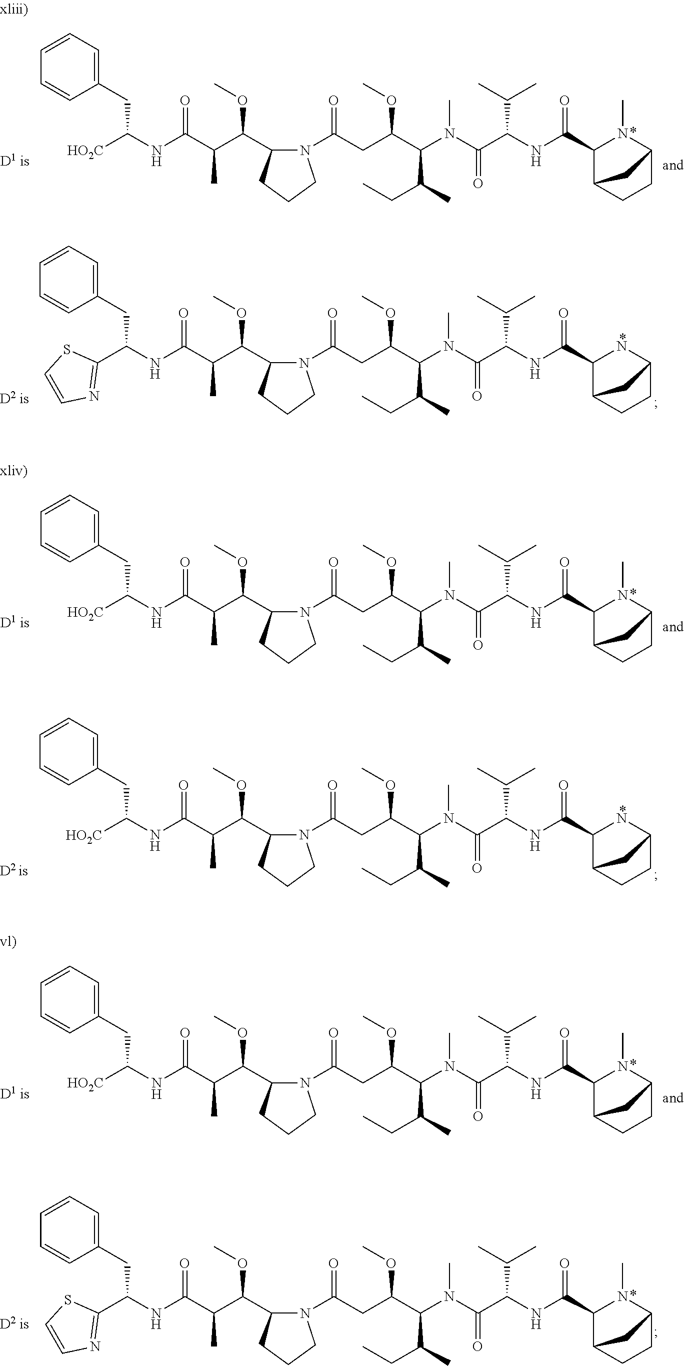

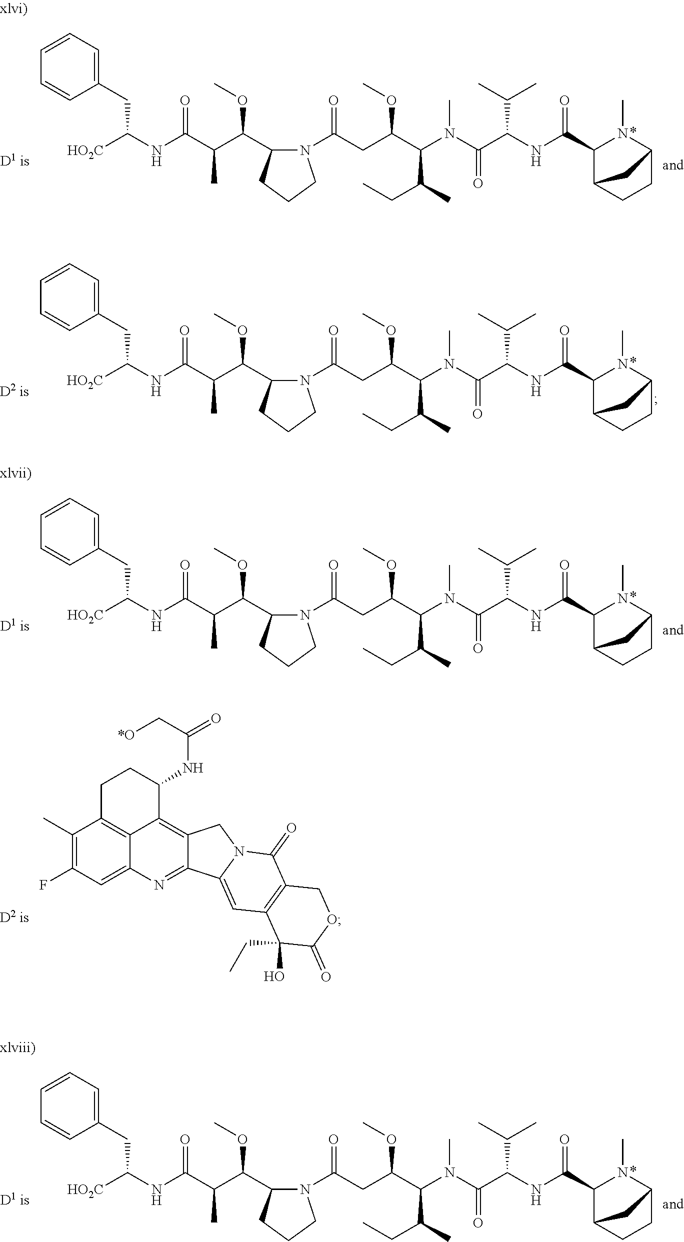

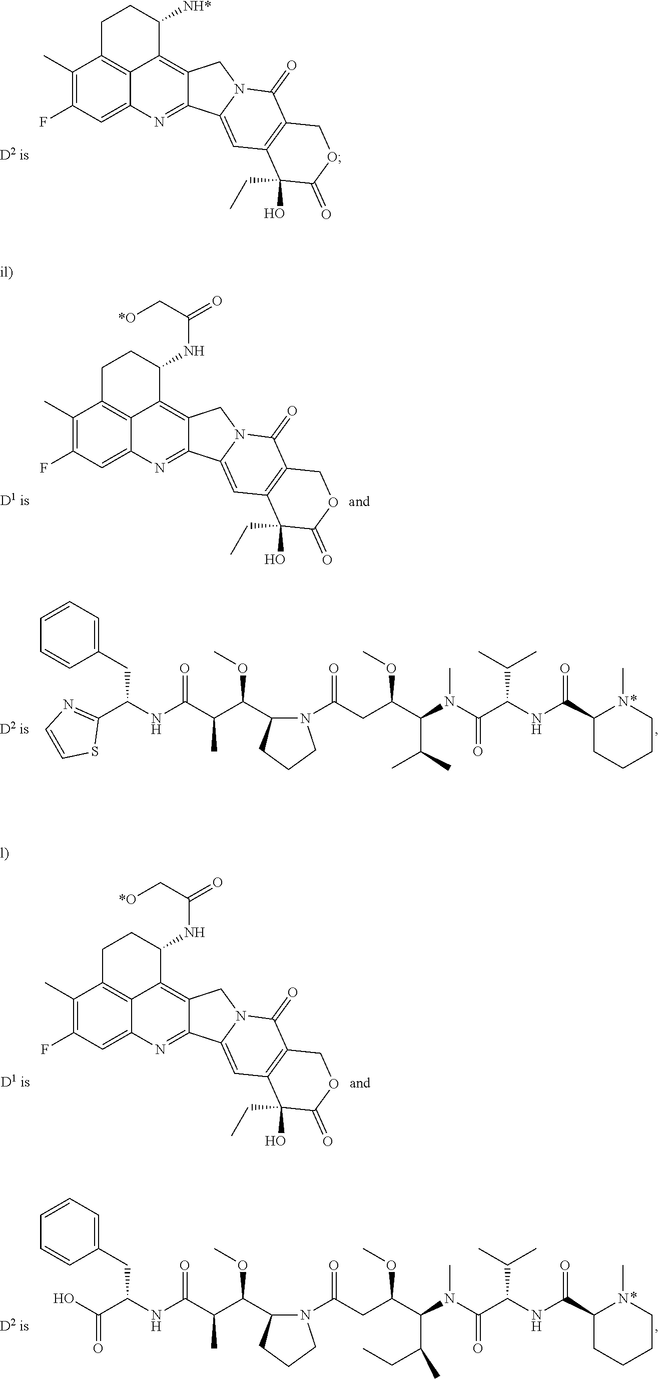

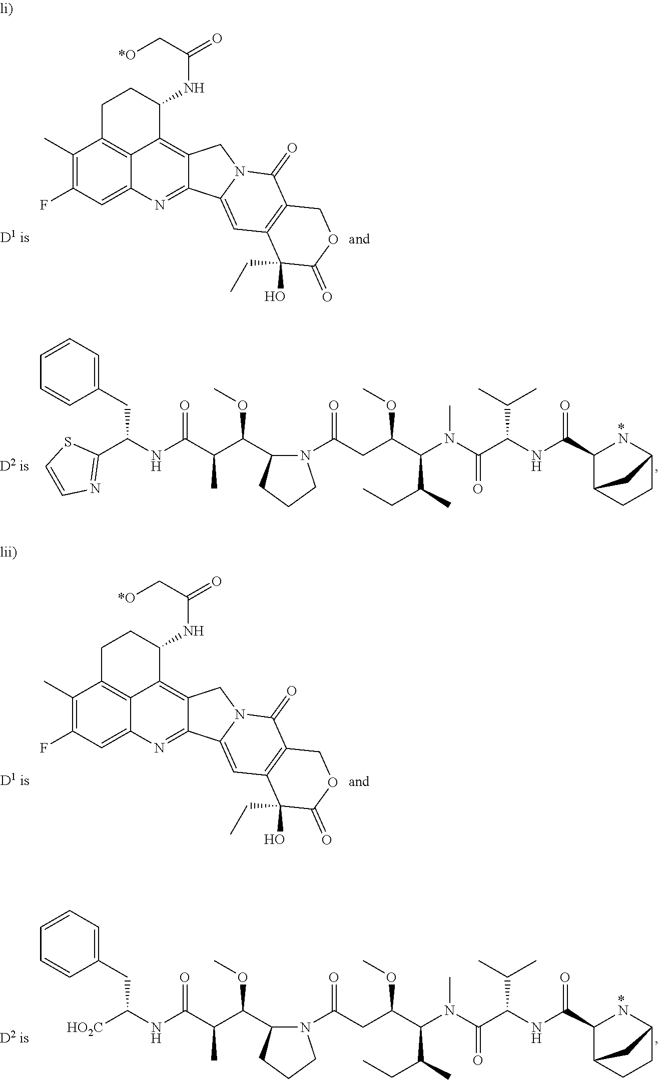

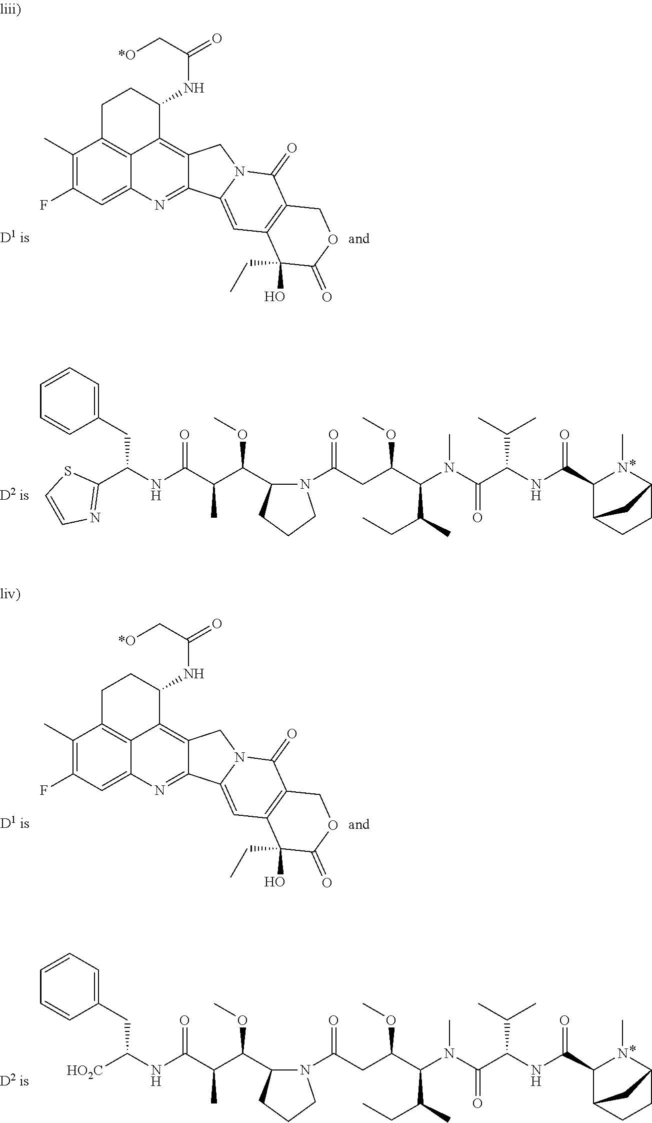

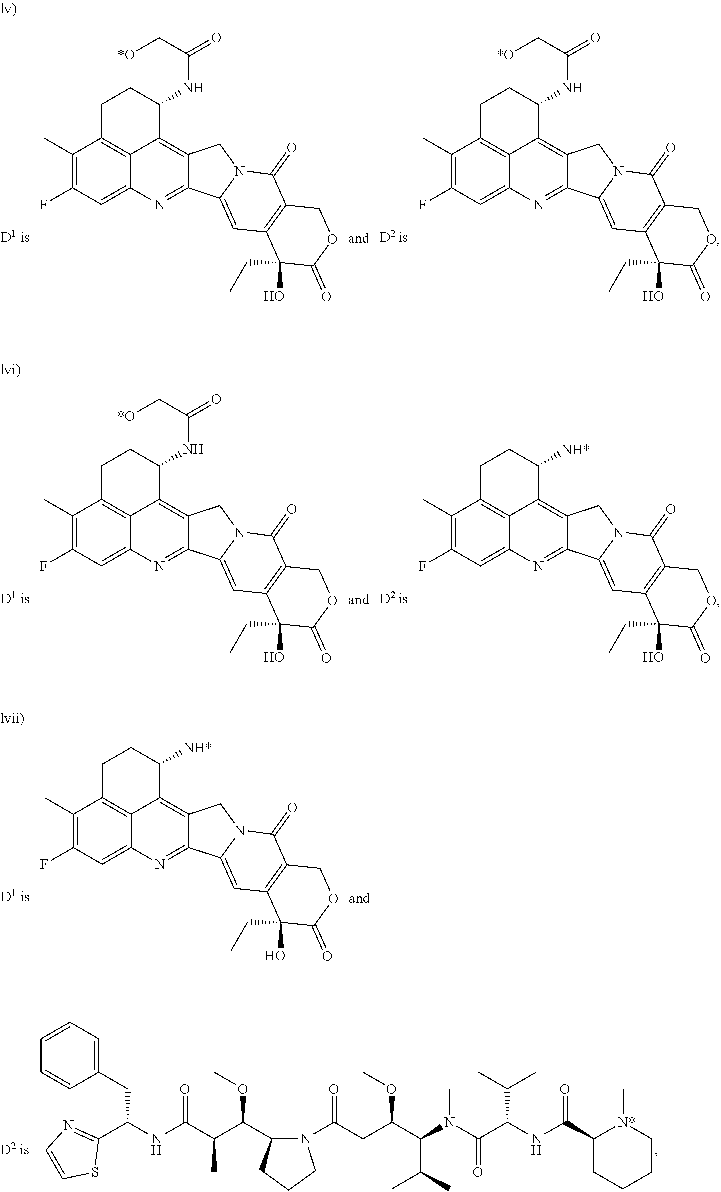

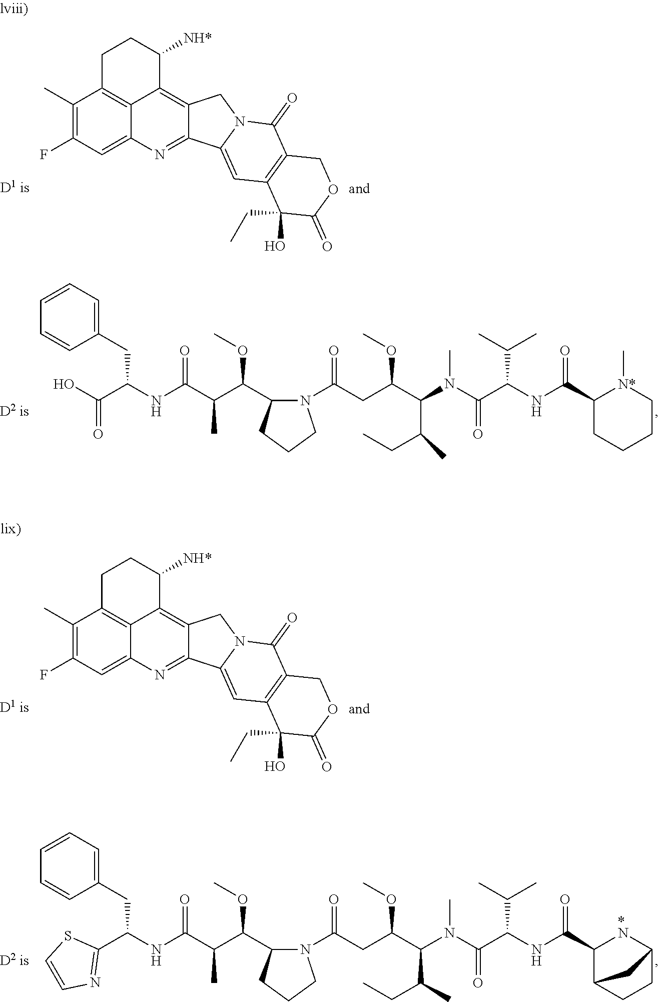

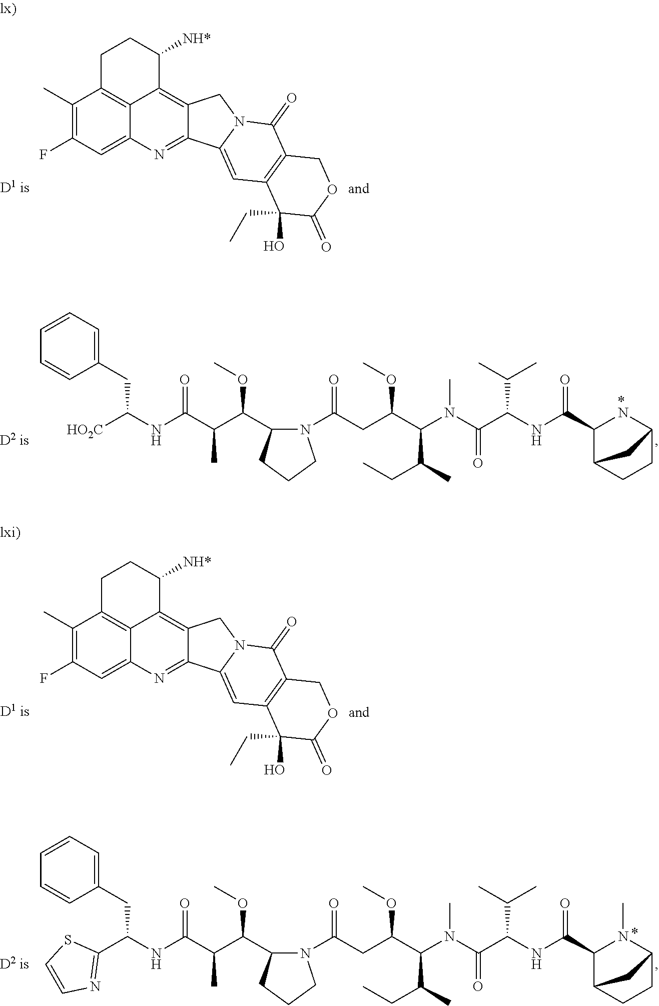

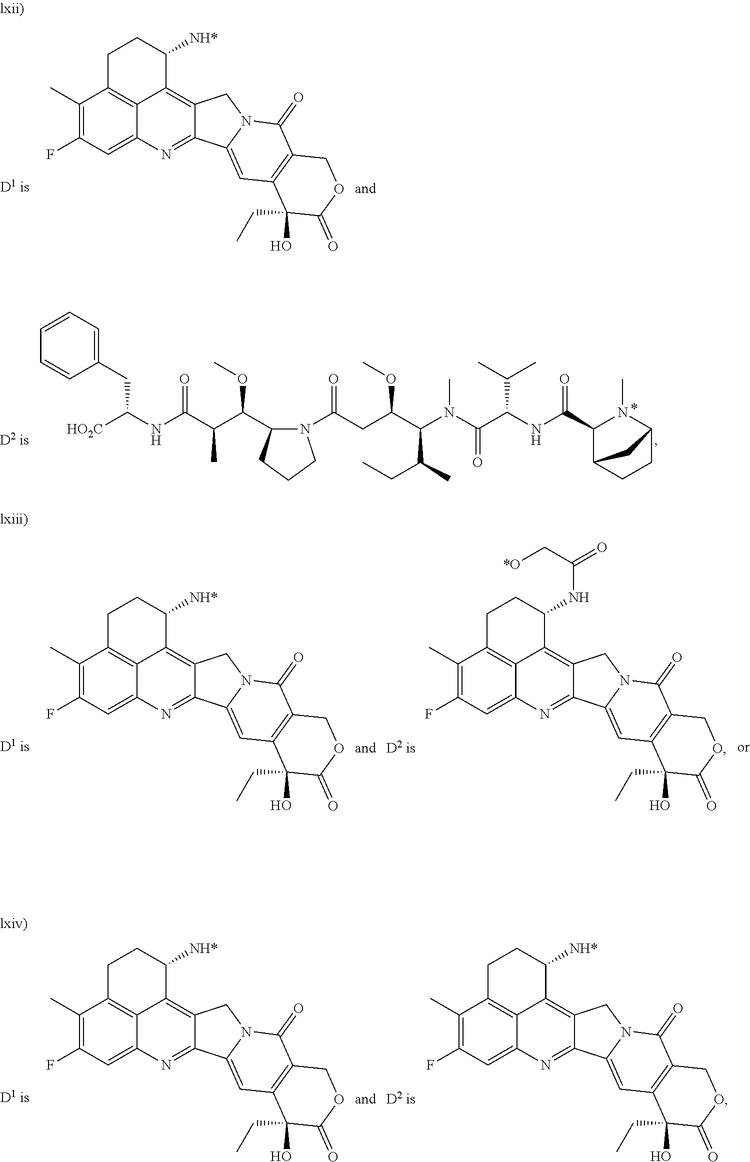

wherein * indicates the point of connection to the remainder of the molecule.
Embodiment 39c. The compound or pharmaceutically acceptable salt according to any one of Embodiments 1 to 37, wherein:

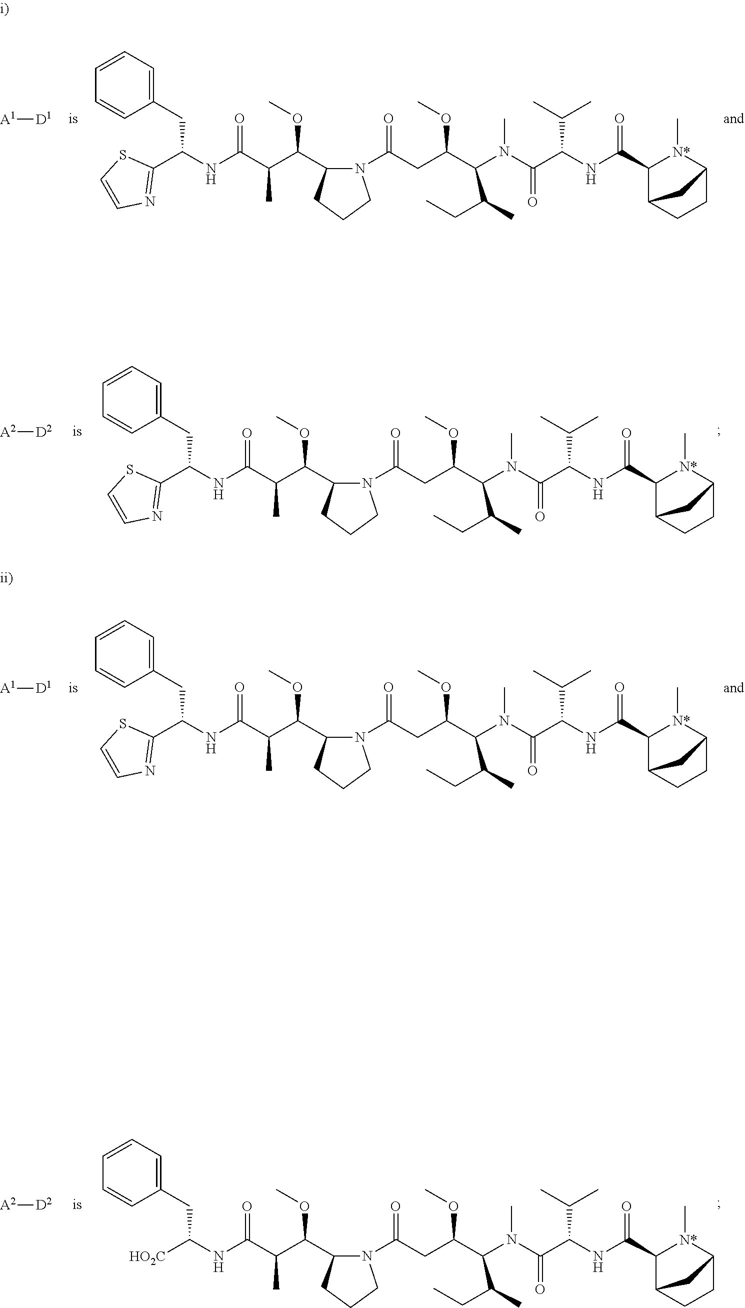

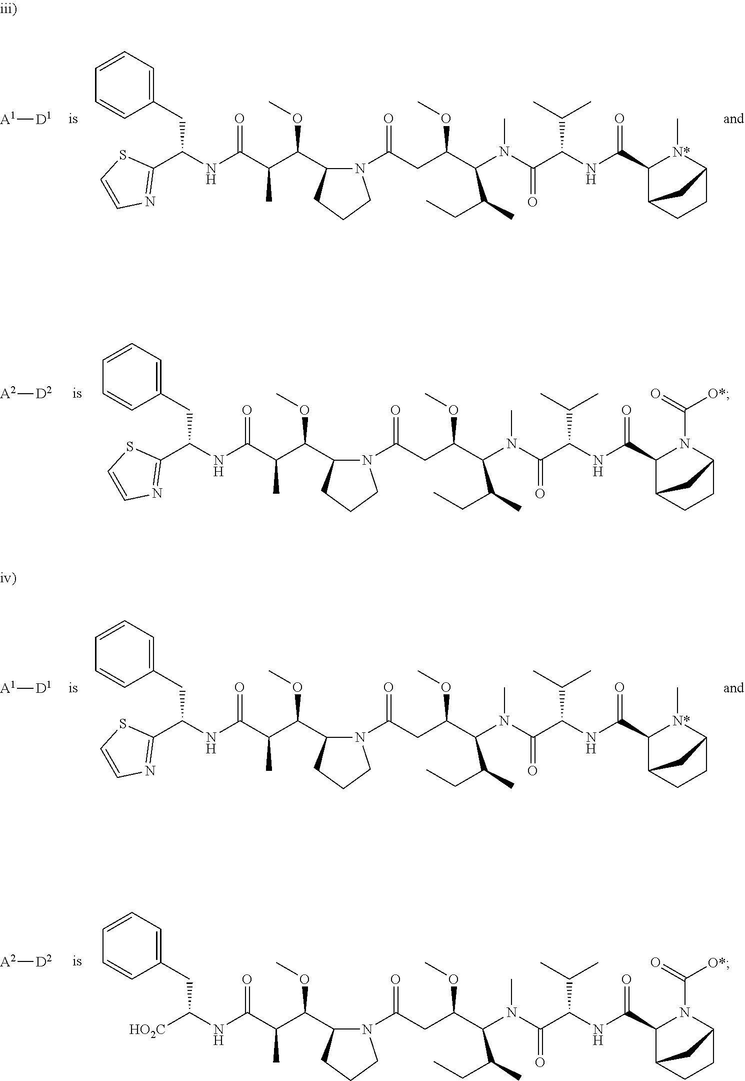

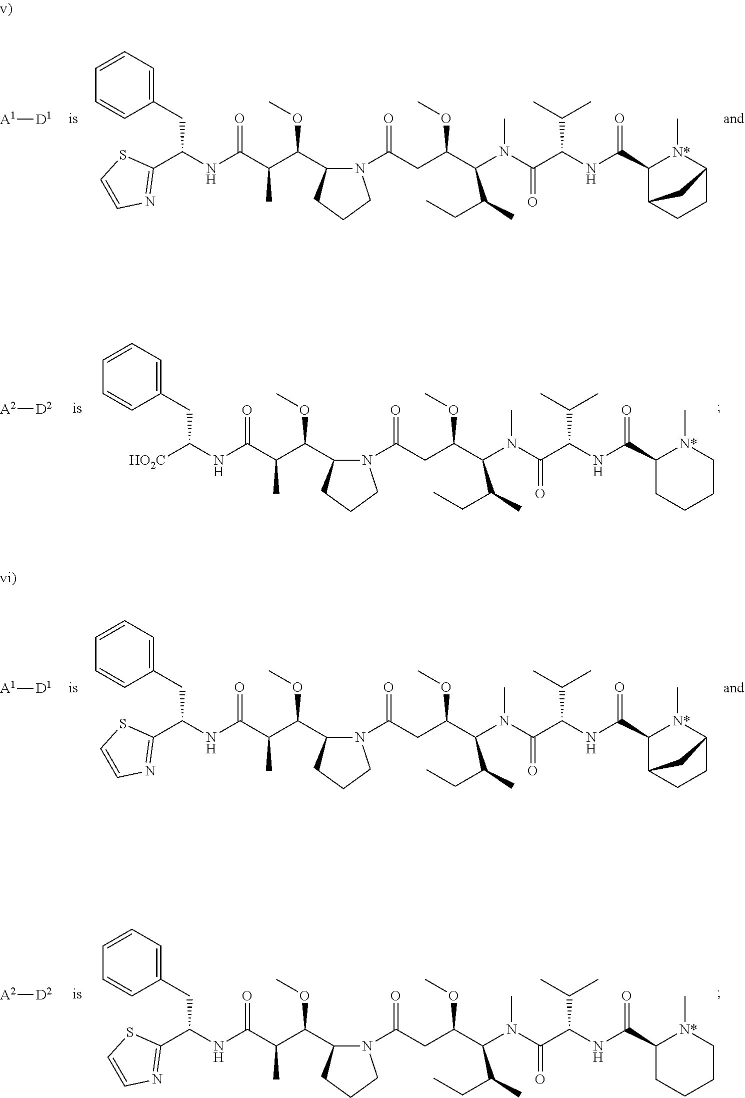

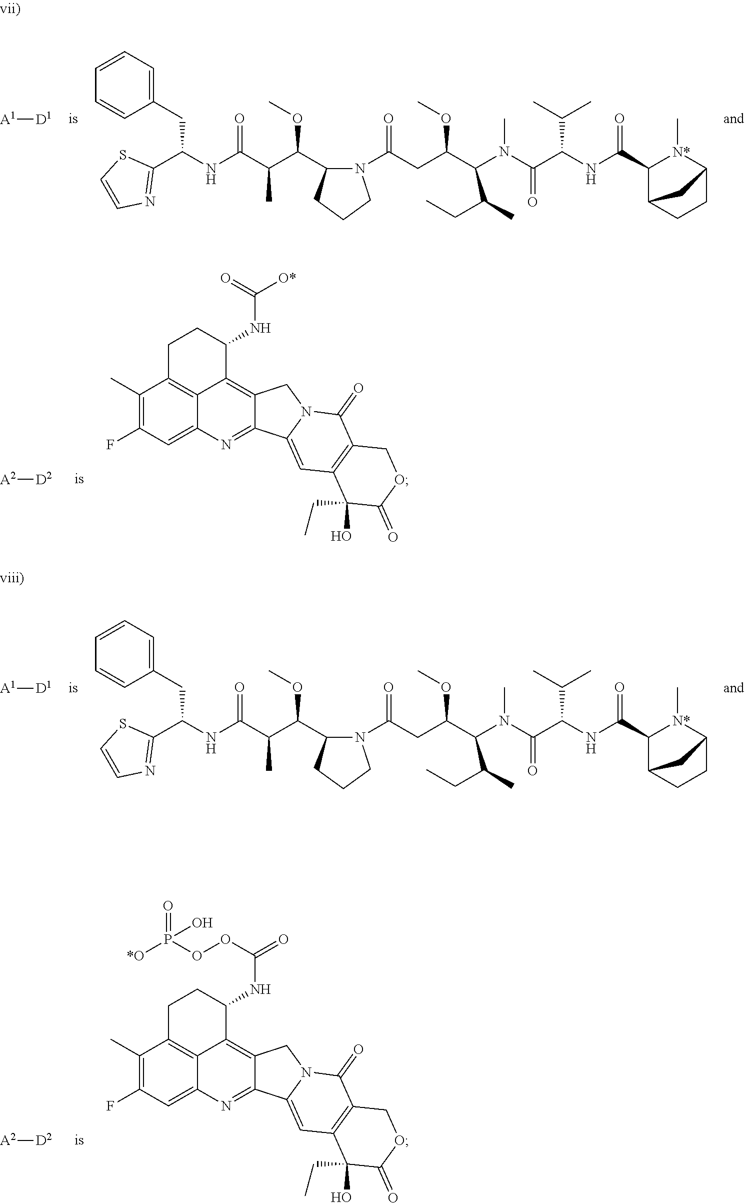

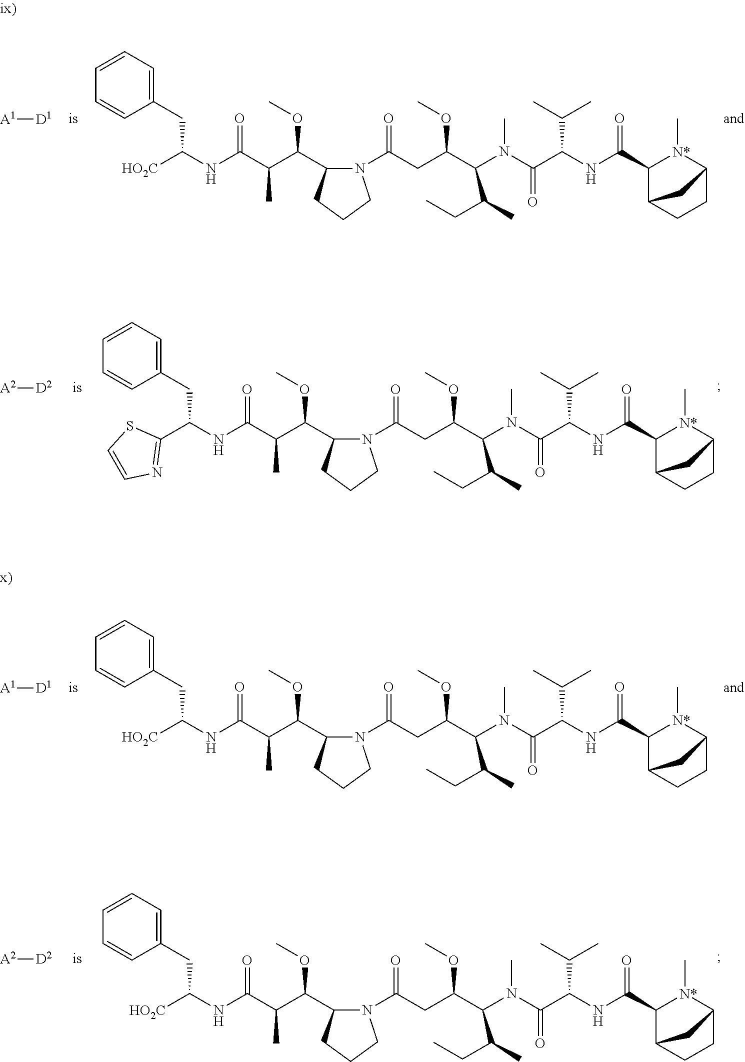

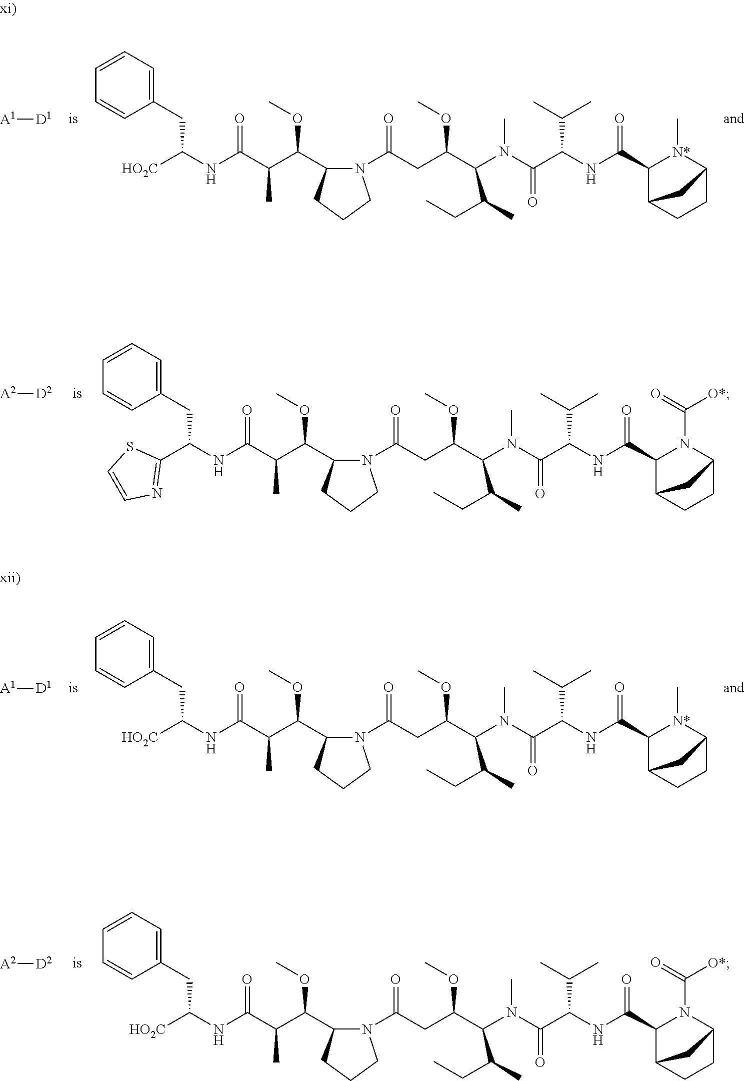

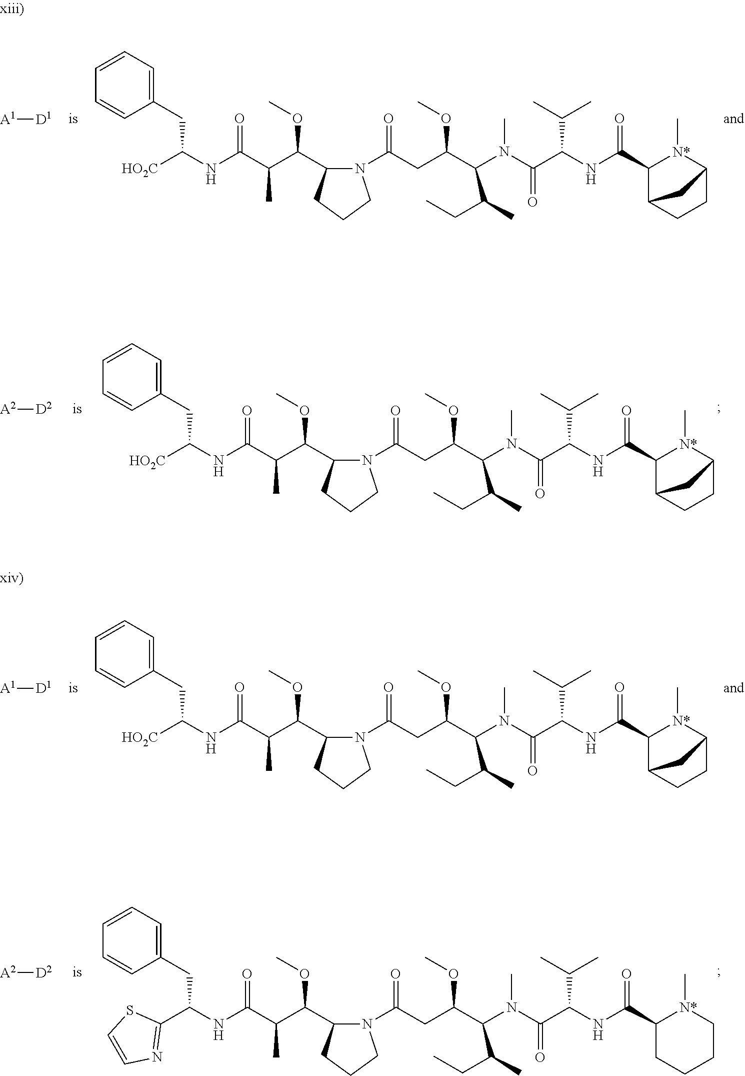

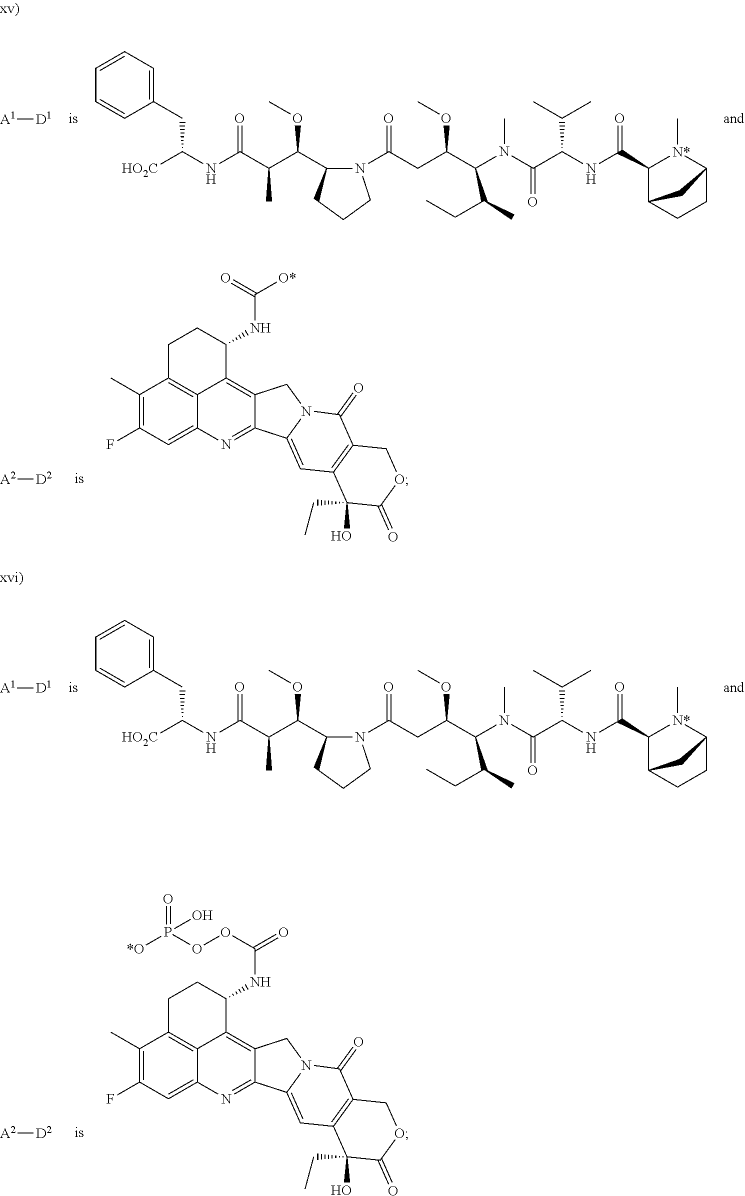

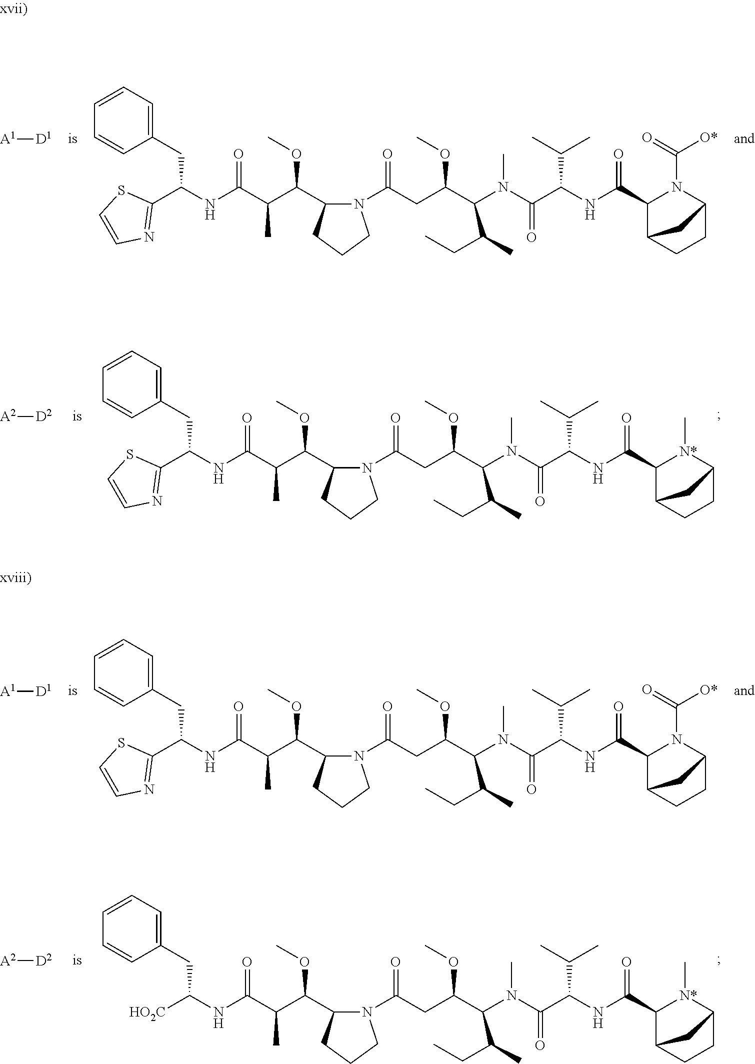

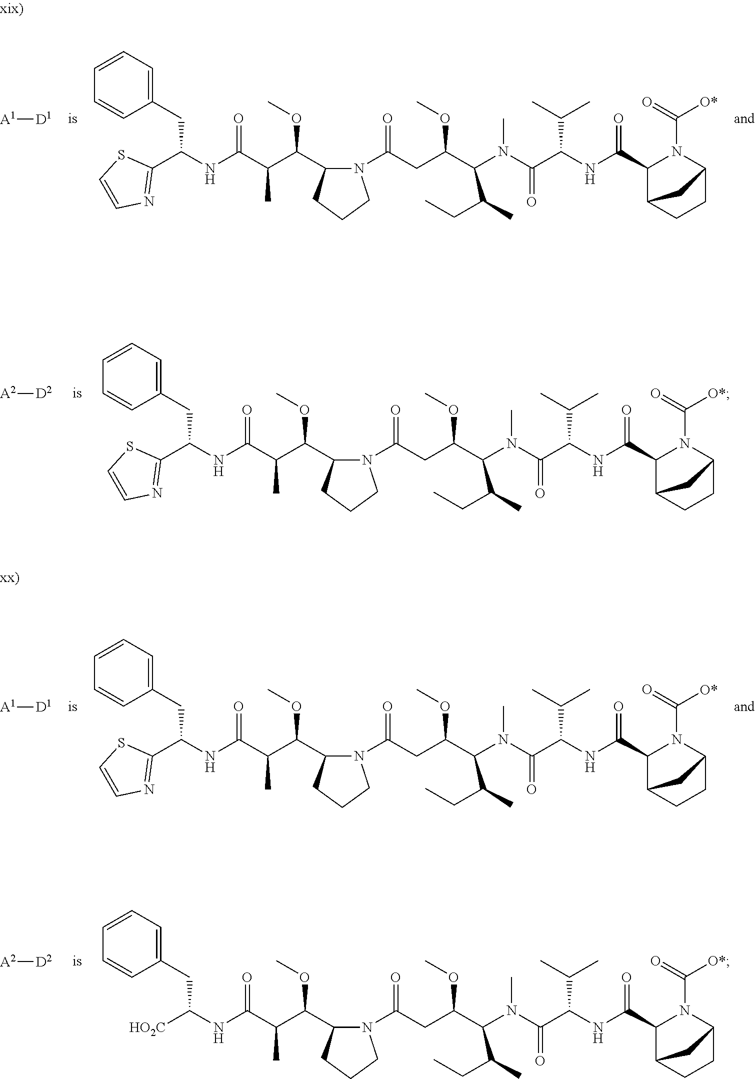

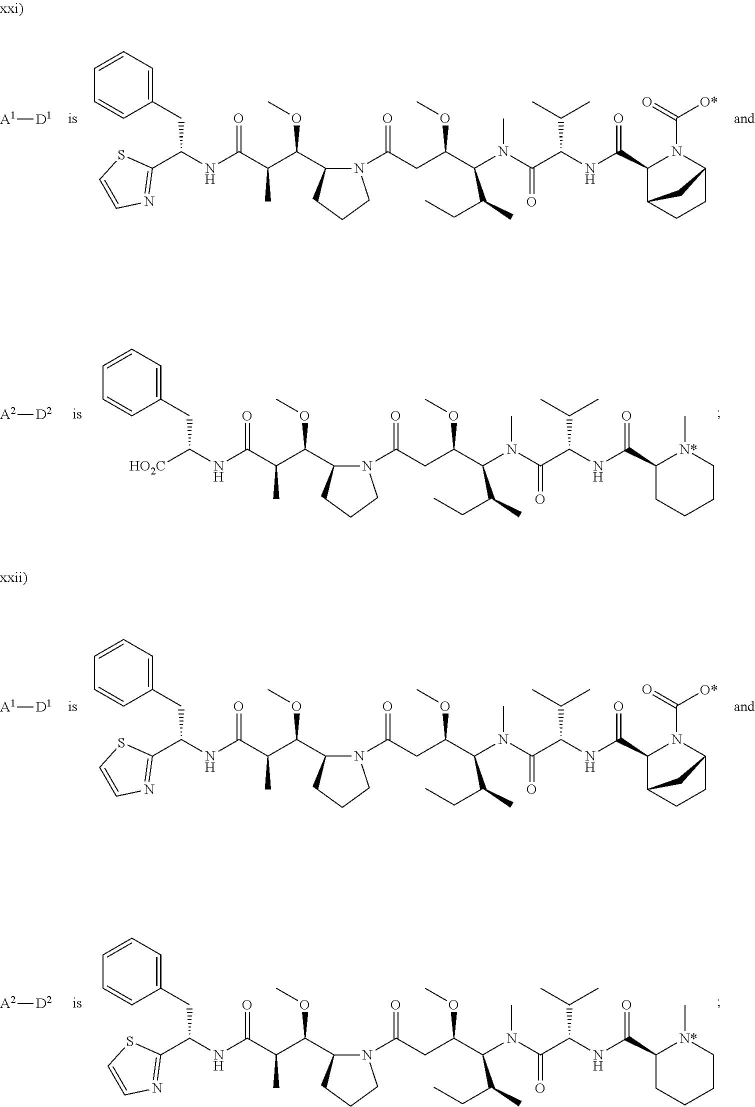

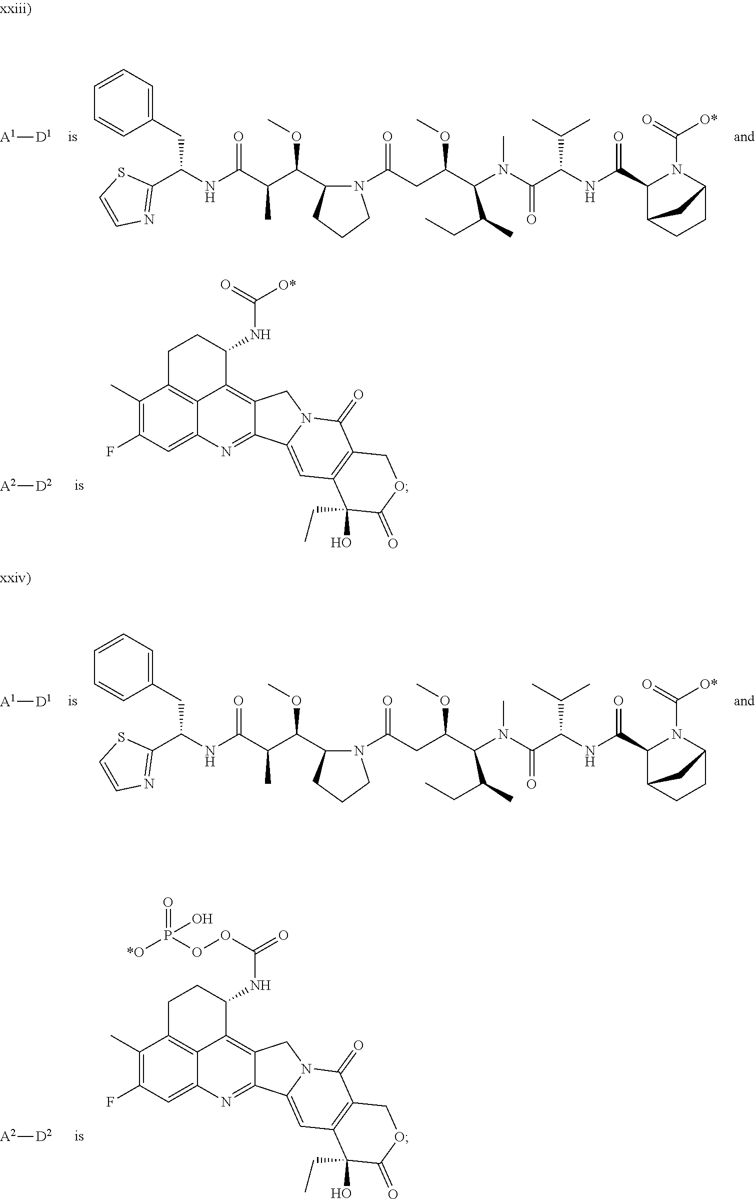

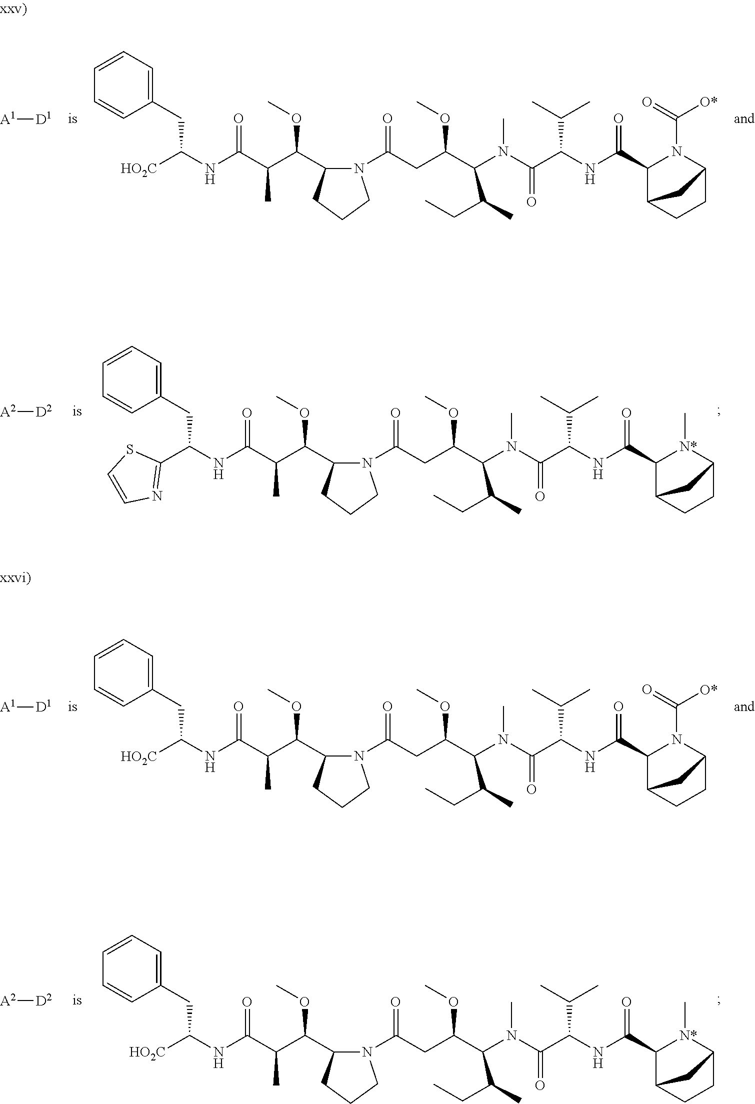

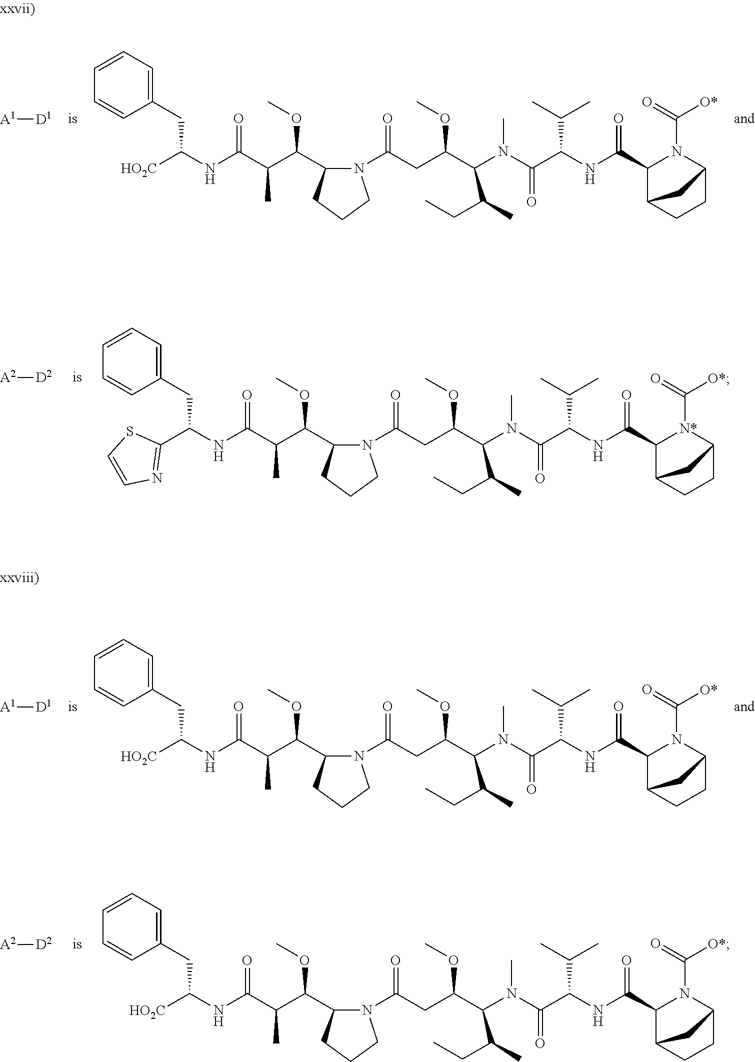

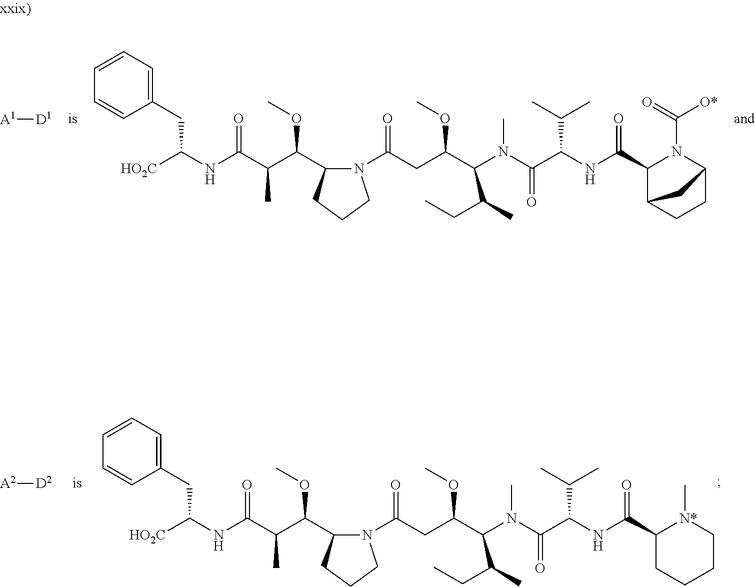

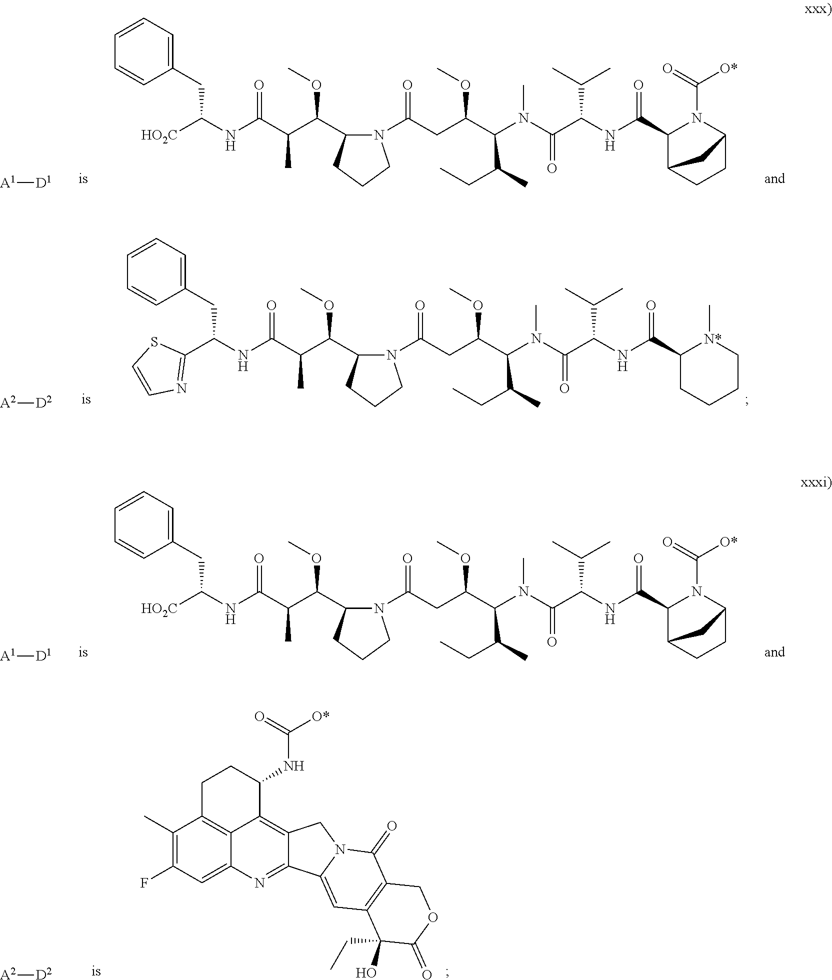

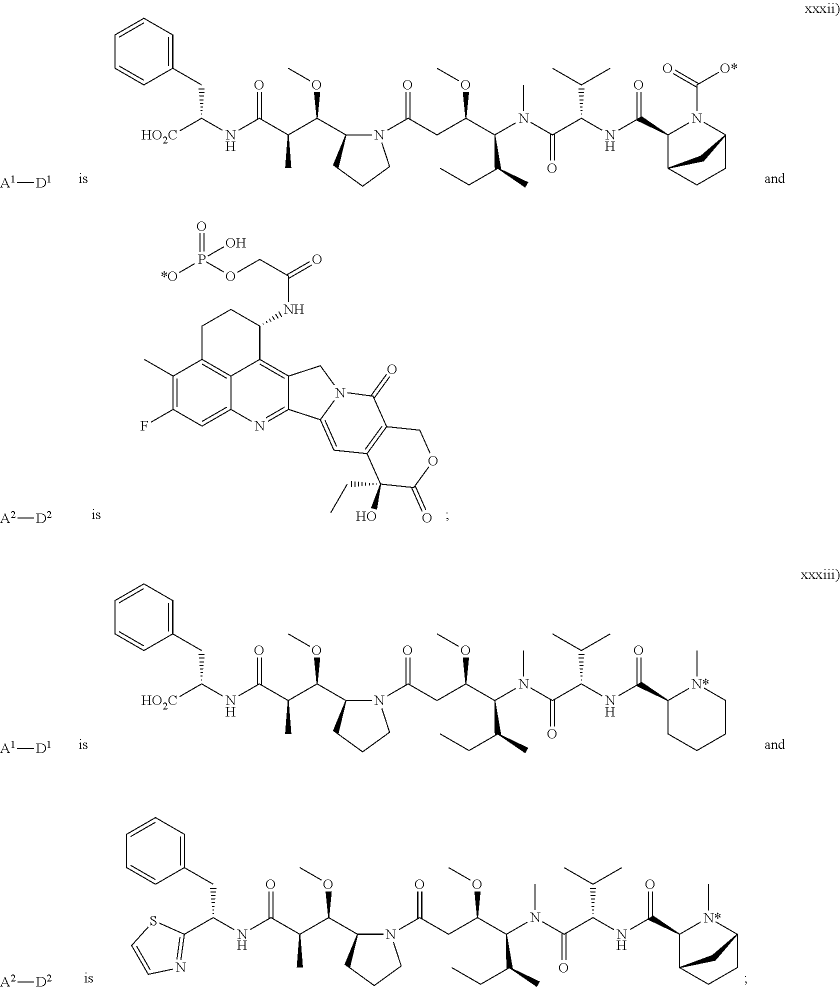

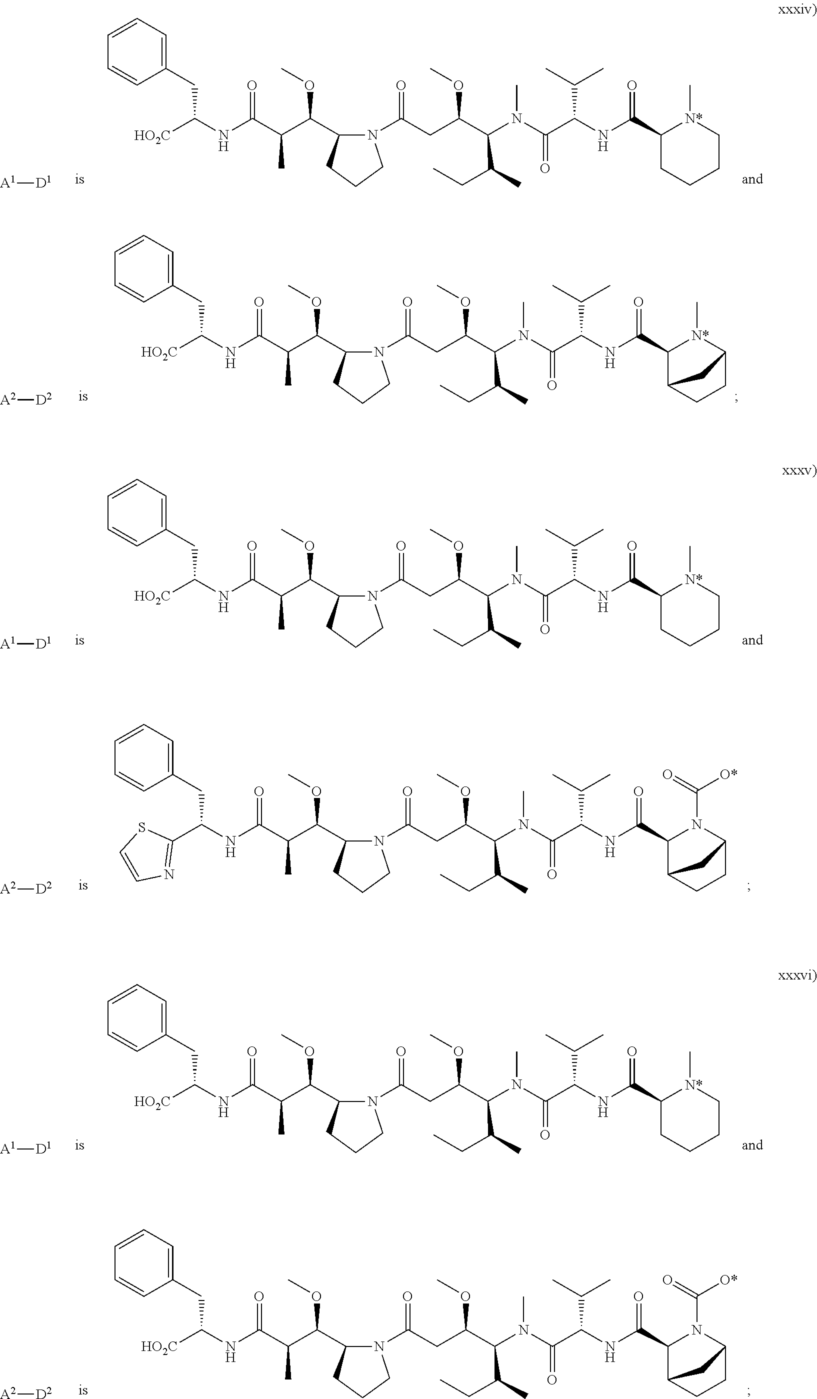

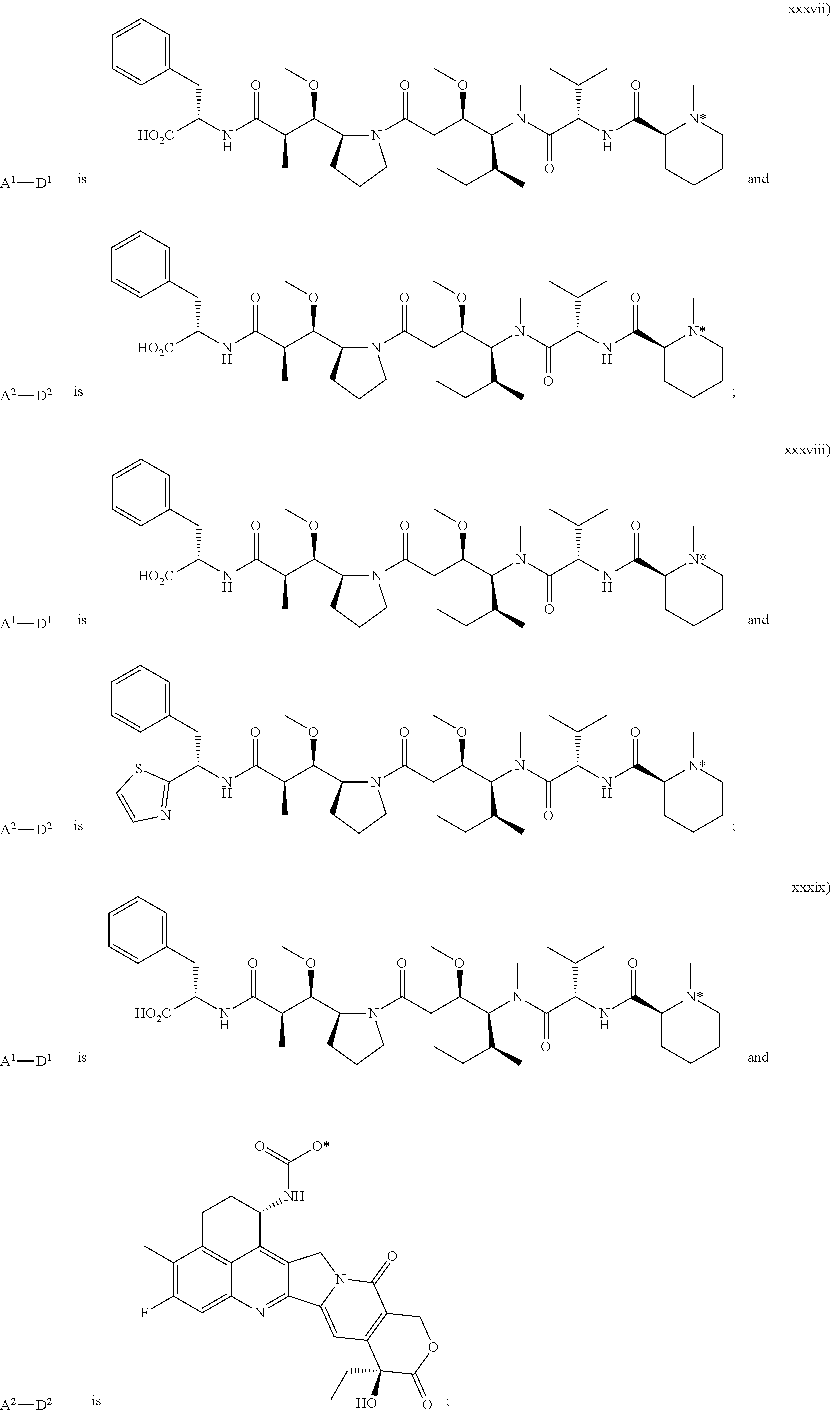

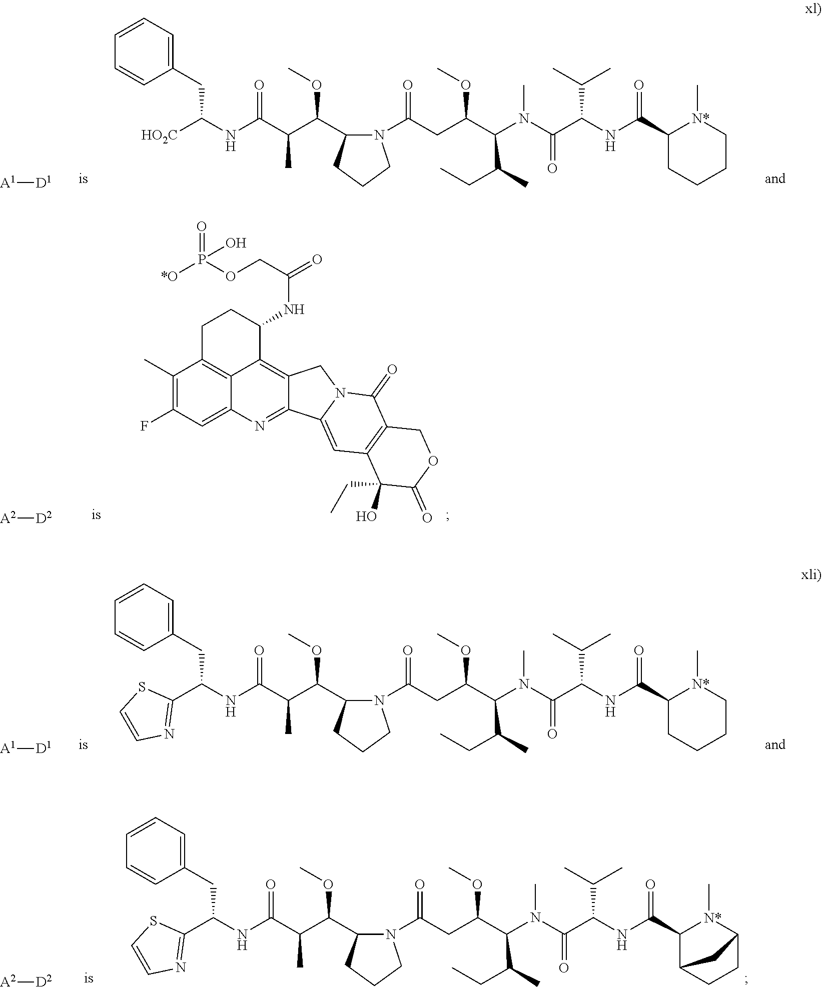

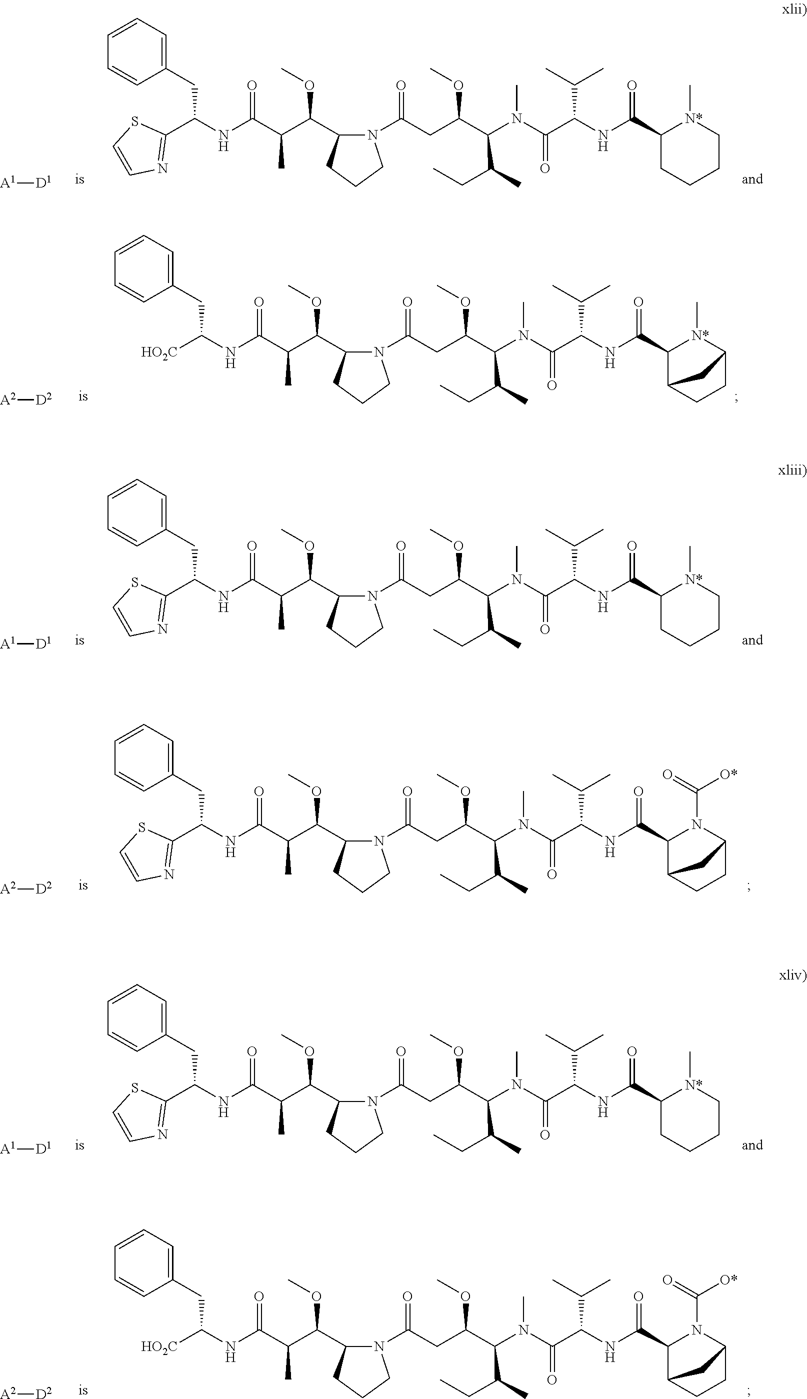

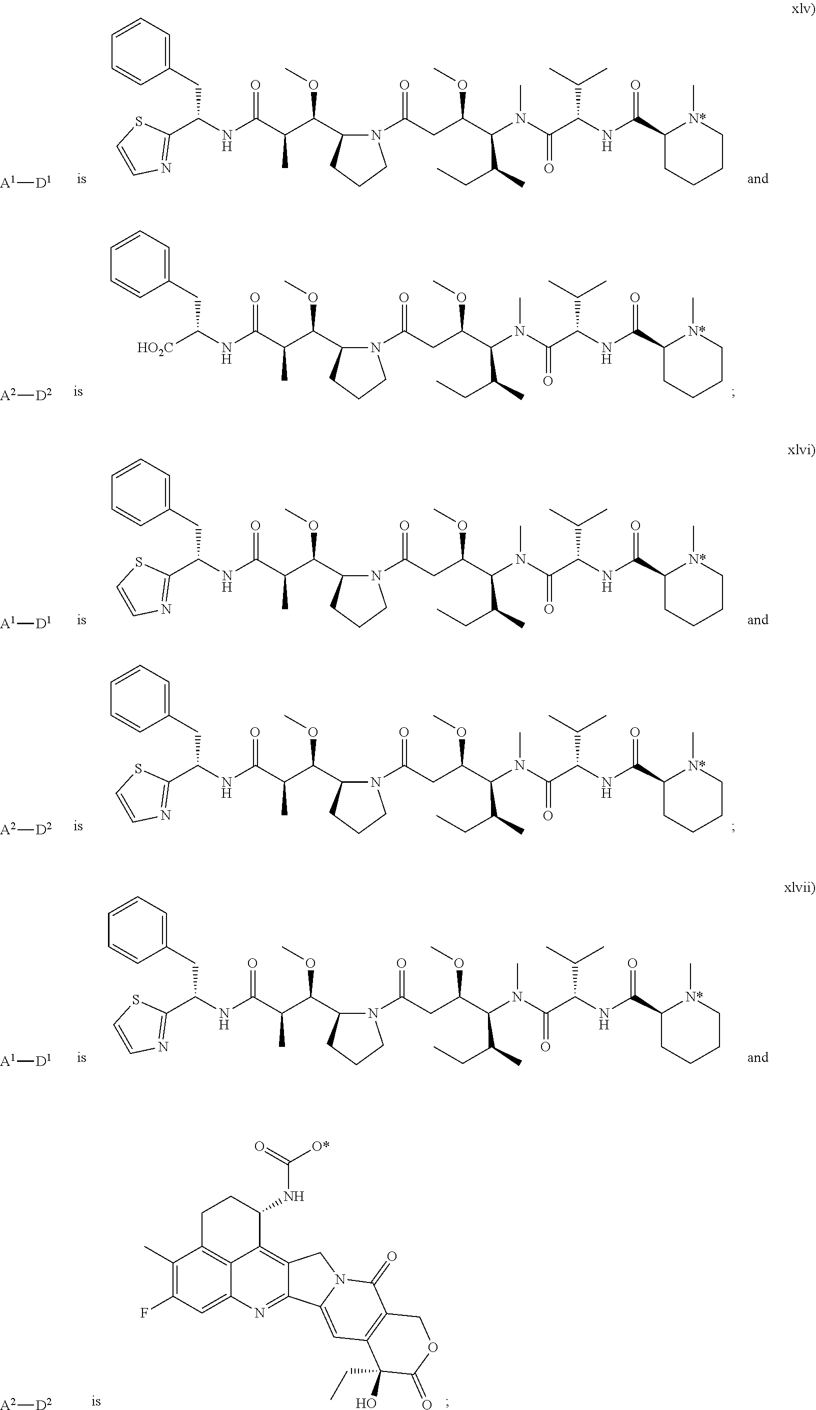

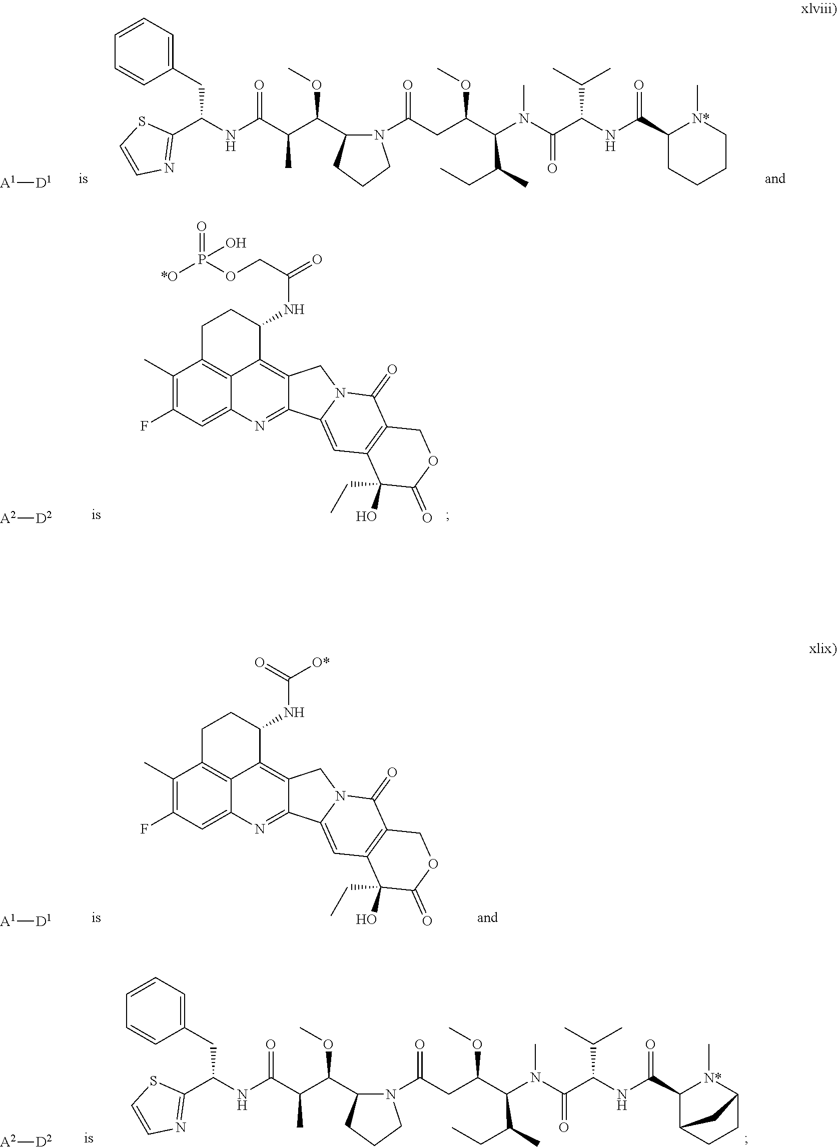

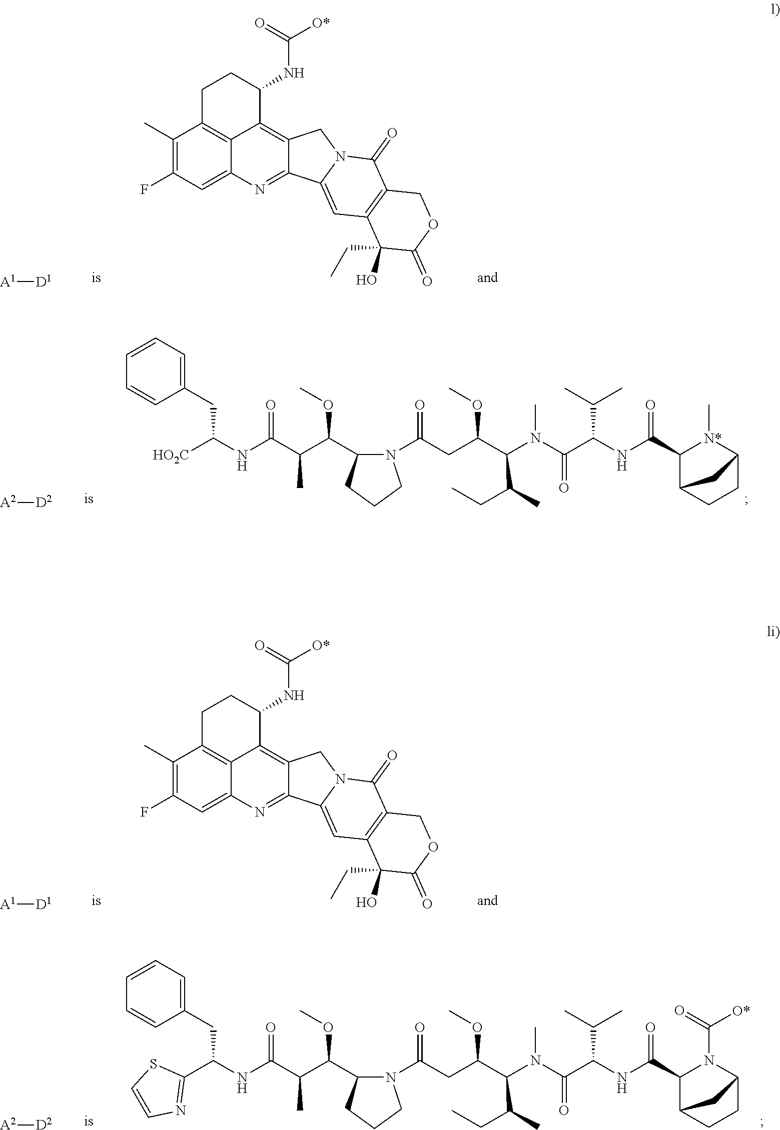

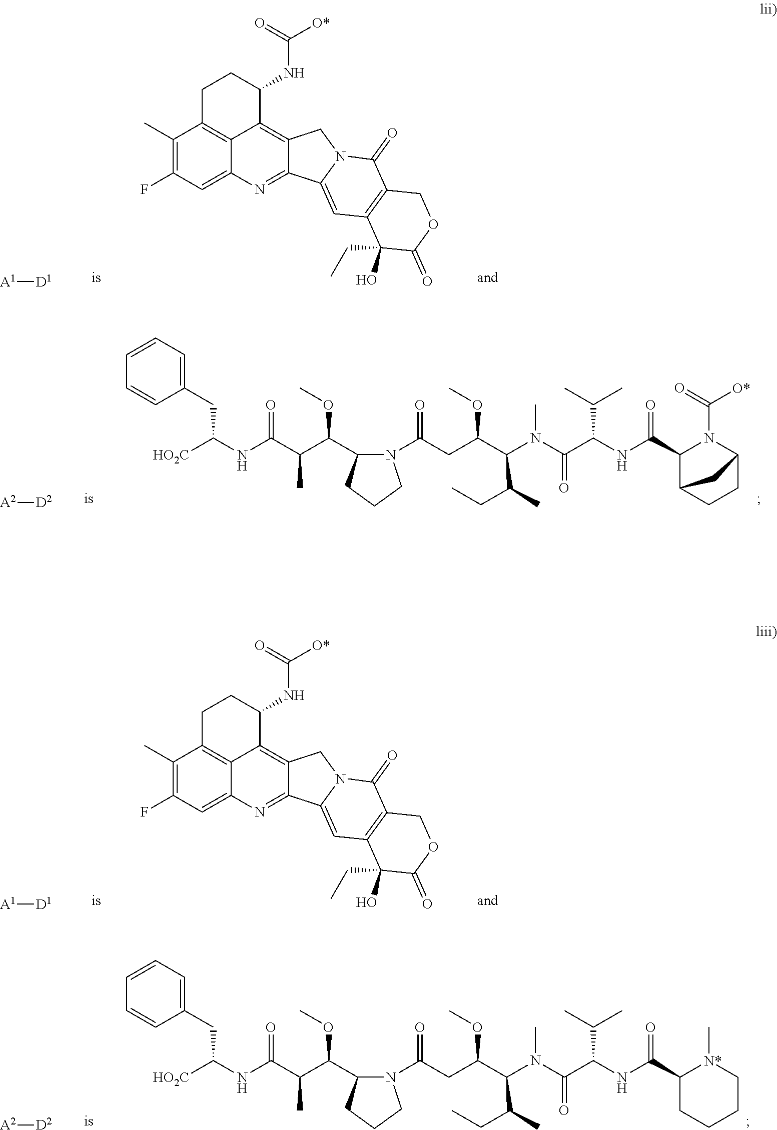

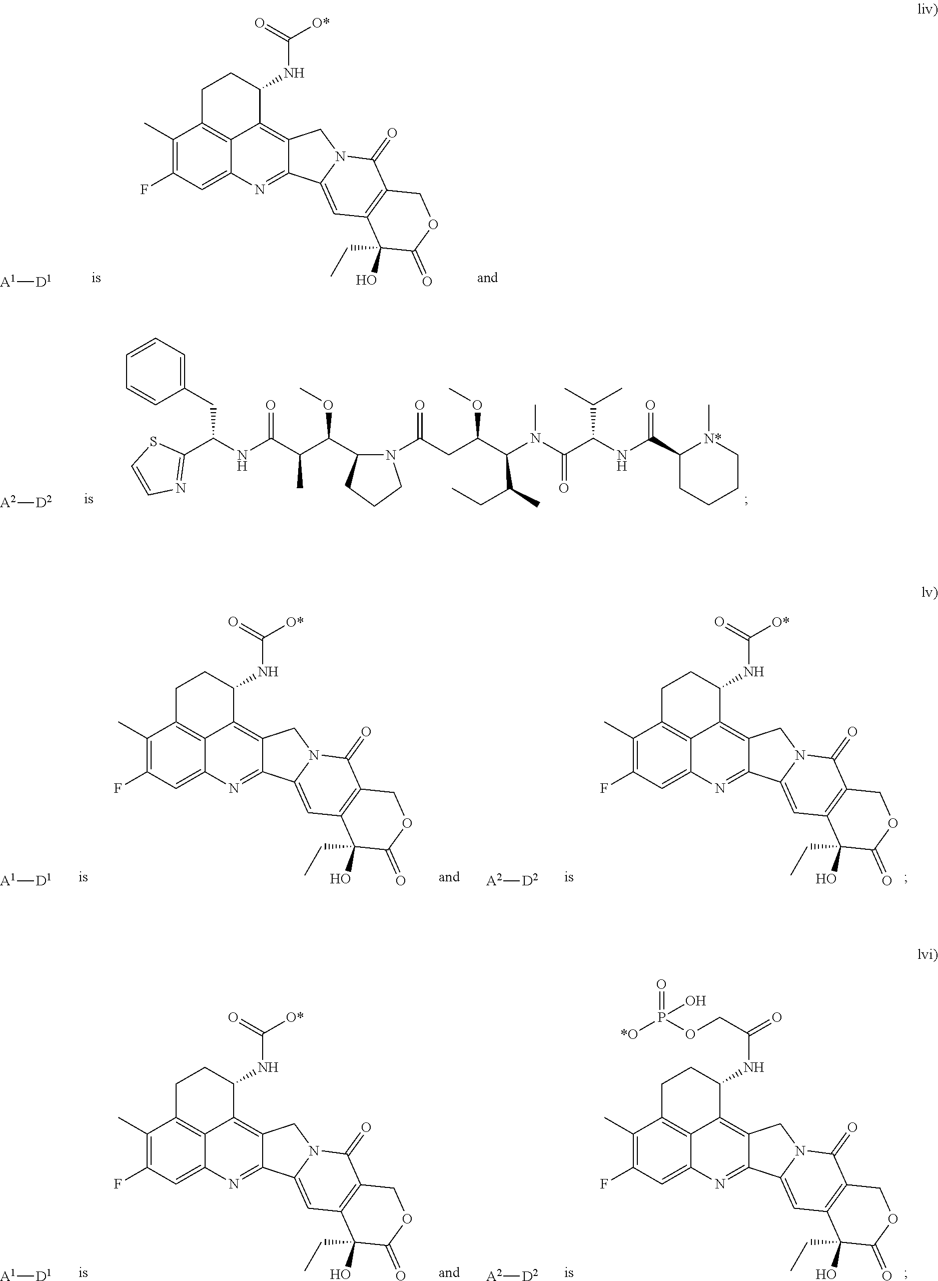

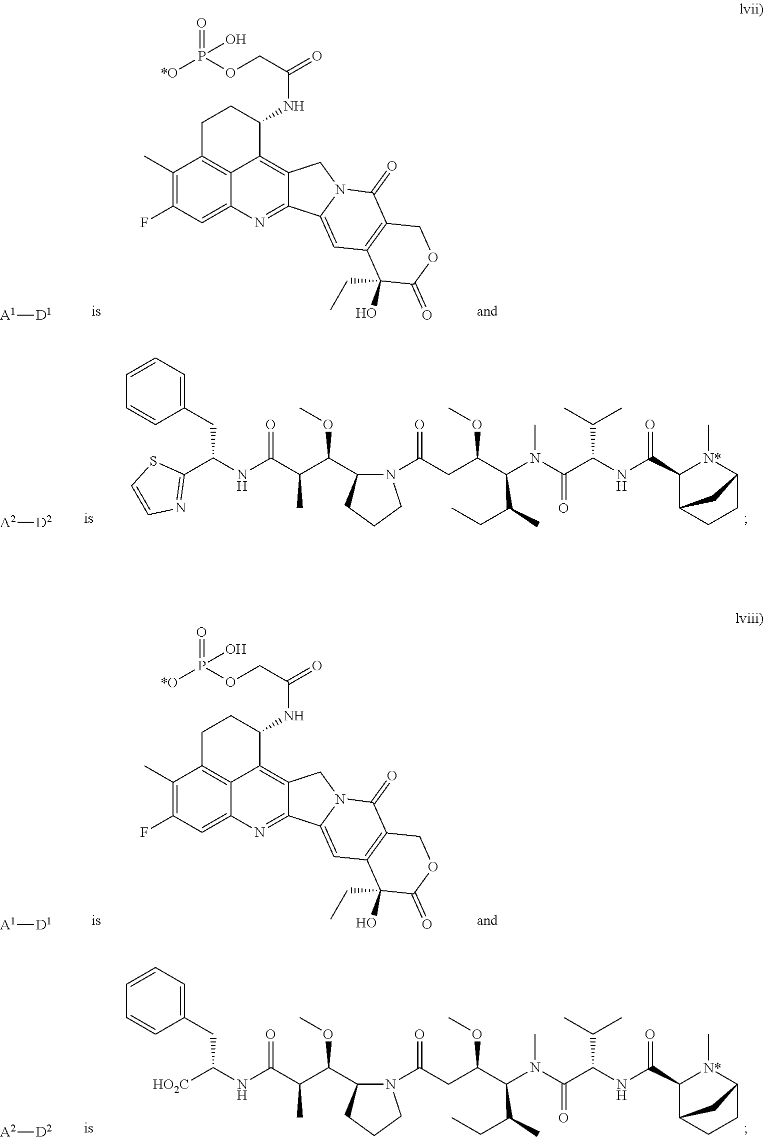

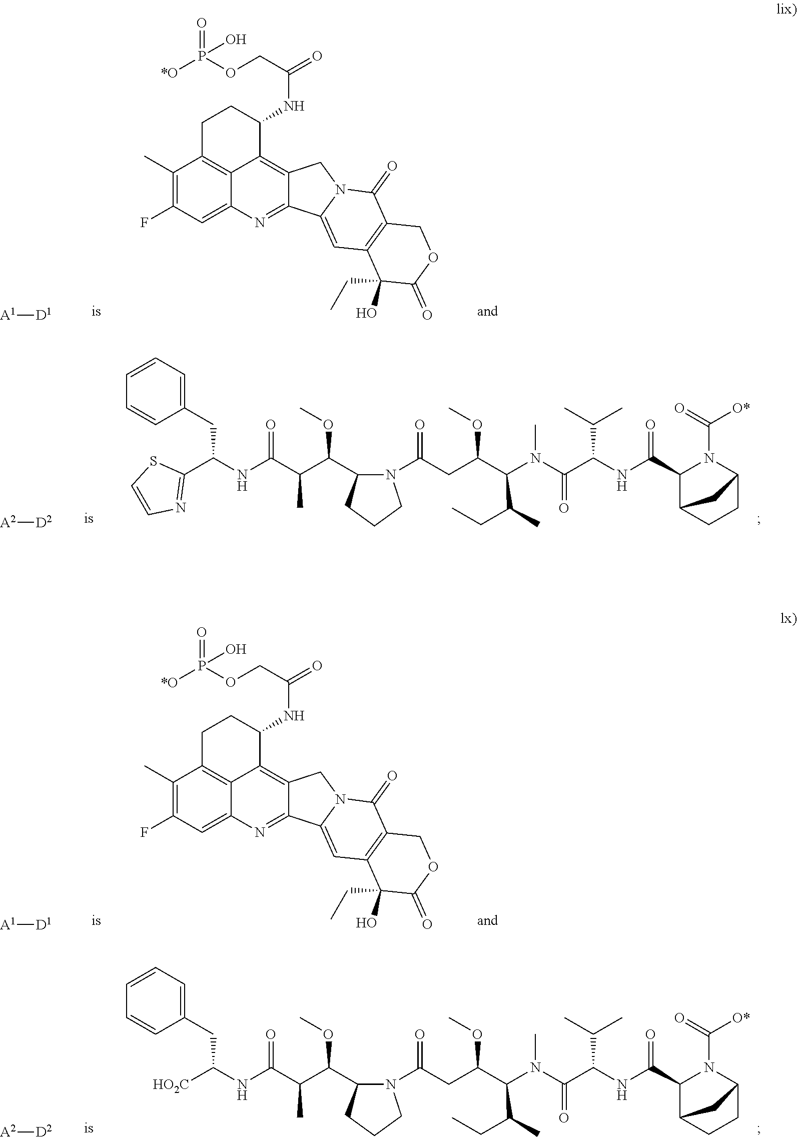

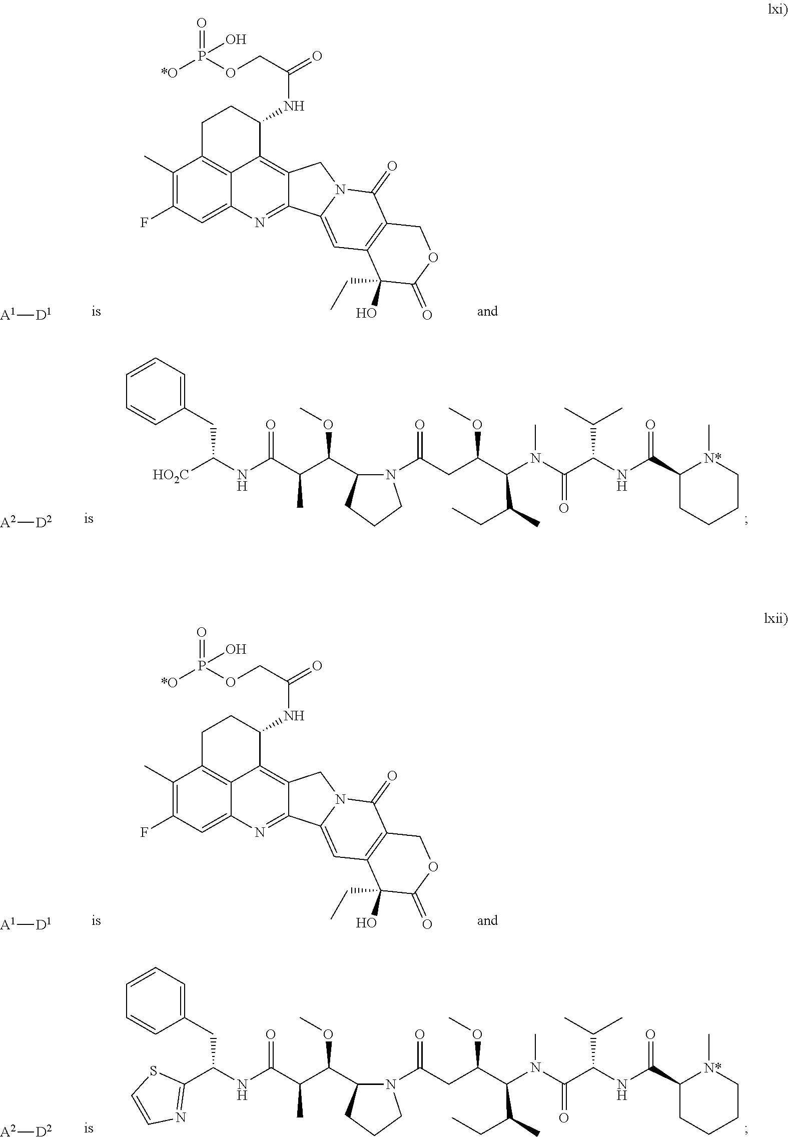

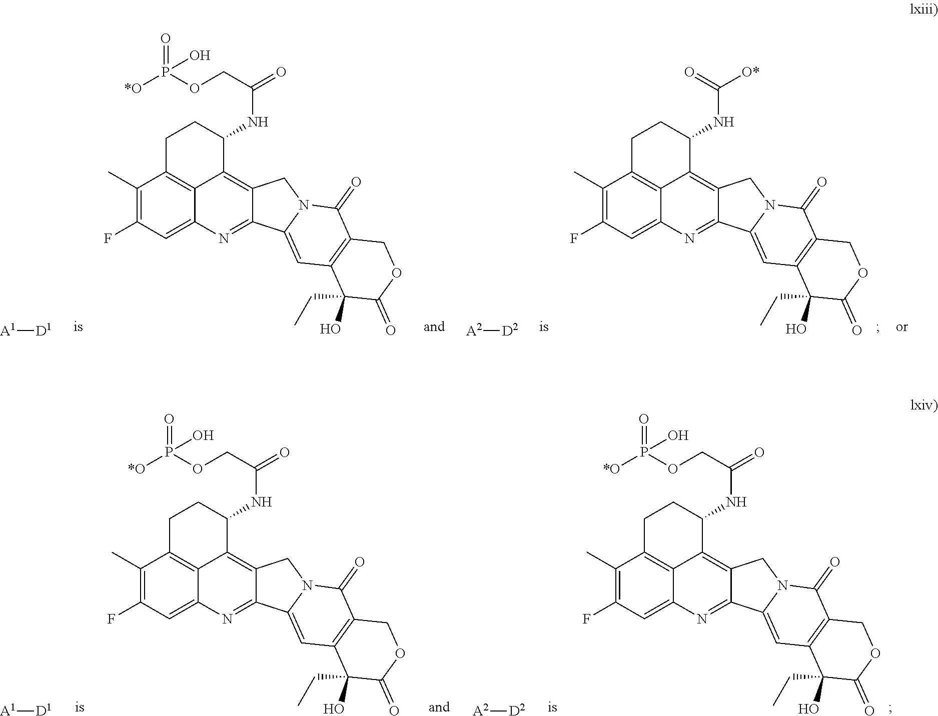

wherein * indicates the point of connecting to the remainder of the molecule.
Embodiment 40. The compound or pharmaceutically acceptable salt according to any one of Embodiments 1 to 37, wherein both D¹ and D² are an auristatin.
Embodiment 40a. The compound or pharmaceutically acceptable salt according to Embodiment 40, wherein both D¹ and D² are an auristatin.
Embodiment 40b. The compound or pharmaceutically acceptable salt according to any one of Embodiments 1 to 37, wherein D¹ and/or D² are a topoisomerase 1 inhibitor.
Embodiment 40c. The compound or pharmaceutically acceptable salt according to Embodiment 40b, wherein both D¹ and D² are a topoisomerase 1 inhibitor.
Embodiment 40d. The compound or pharmaceutically acceptable salt according to any one of Embodiments 1 to 37, wherein D¹ and D² is an auristatin and the other of D¹ and D² is a topoisomerase 1 inhibitor.
Embodiment 40e. The compound or pharmaceutically acceptable salt according to Embodiment 40d, wherein D¹ is an auristatin and D² is a topoisomerase 1 inhibitor.
Embodiment 40f. The compound or pharmaceutically acceptable salt according to Embodiment 40d, wherein D¹ is a topoisomerase 1 inhibitor and D² is an auristatin.
Embodiment 41. The compound or pharmaceutically acceptable salt according to any one of the preceding Embodiments wherein D¹ and D² are independently selected from the group consisting of

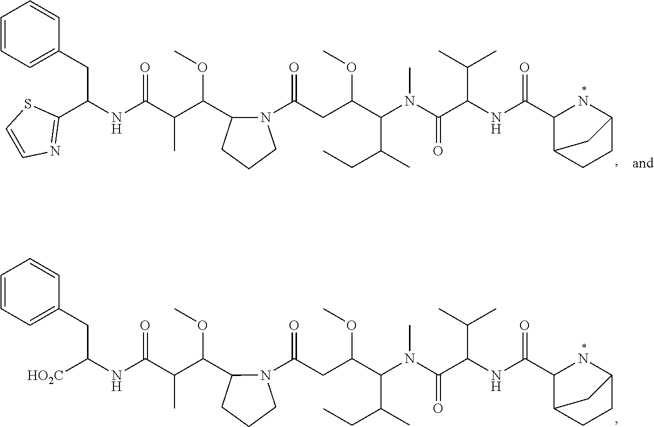

wherein * indicates the point of attachment to A¹ or A²; or a pharmaceutically acceptable salt thereof.
Embodiment 41a. The compound or pharmaceutically acceptable salt according to Embodiment 41, wherein D¹ and D² are independently selected from the group consisting of:

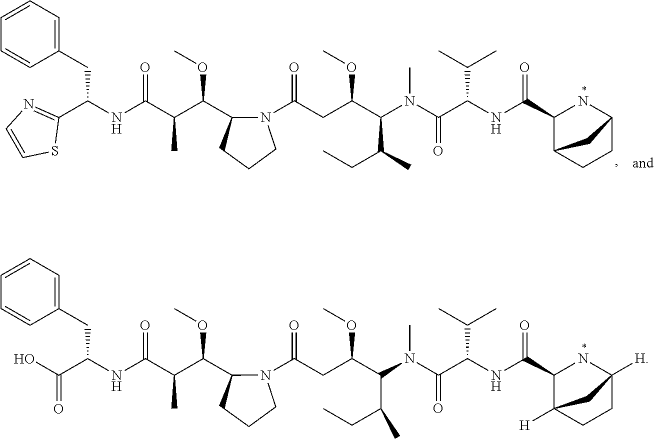

Embodiment 41b. The compound or pharmaceutically acceptable salt according to Embodiment 39a, wherein D¹ and D² are independently selected from the group consisting of:

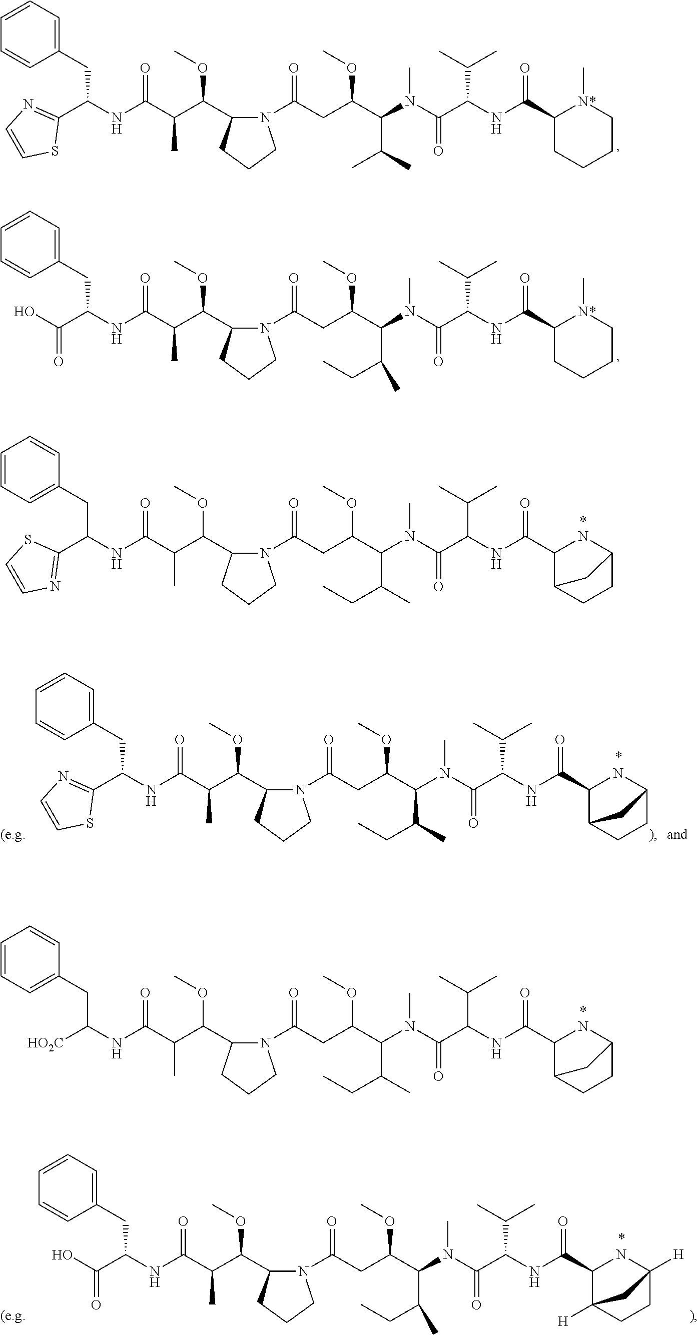

wherein * indicates the point of attachment to A¹ or A²; or a pharmaceutically acceptable salt thereof.
Embodiment 41c. The compound or pharmaceutically acceptable salt according to Embodiment 39a, wherein D¹ and D² are independently selected from the group consisting of:

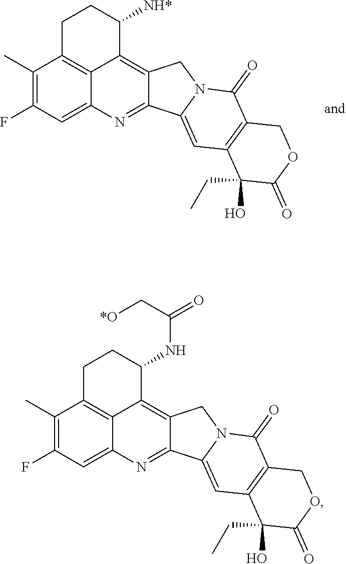

wherein * indicates the point of attachment to A¹ or A²; or a pharmaceutically acceptable salt thereof.
Embodiment 42. The compound or pharmaceutically acceptable salt according to any one of Embodiments 1 to 37, wherein D¹ and D² are each independently selected from an antitubulin agent, a tubulin inhibitor, a DNA minor groove binder, a DNA replication inhibitor, an alkylating agent, an antibiotic, an antifolate, an antimetabolite, a chemotherapy sensitizer, a topoisomerase inhibitor, and/or a vinca alkaloid.
Embodiment 43. The compound or pharmaceutically acceptable salt according to any one of Embodiments 1 to 37, wherein D¹ and D² are each independently selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor, a DHFR inhibitor, or a pro-apoptotic agent, optionally with the proviso that neither D¹ nor D² is a BH3 mimetic.
Embodiment 44. The compound or pharmaceutically acceptable salt thereof according to any one of the preceding Embodiments, selected from the group consisting of:

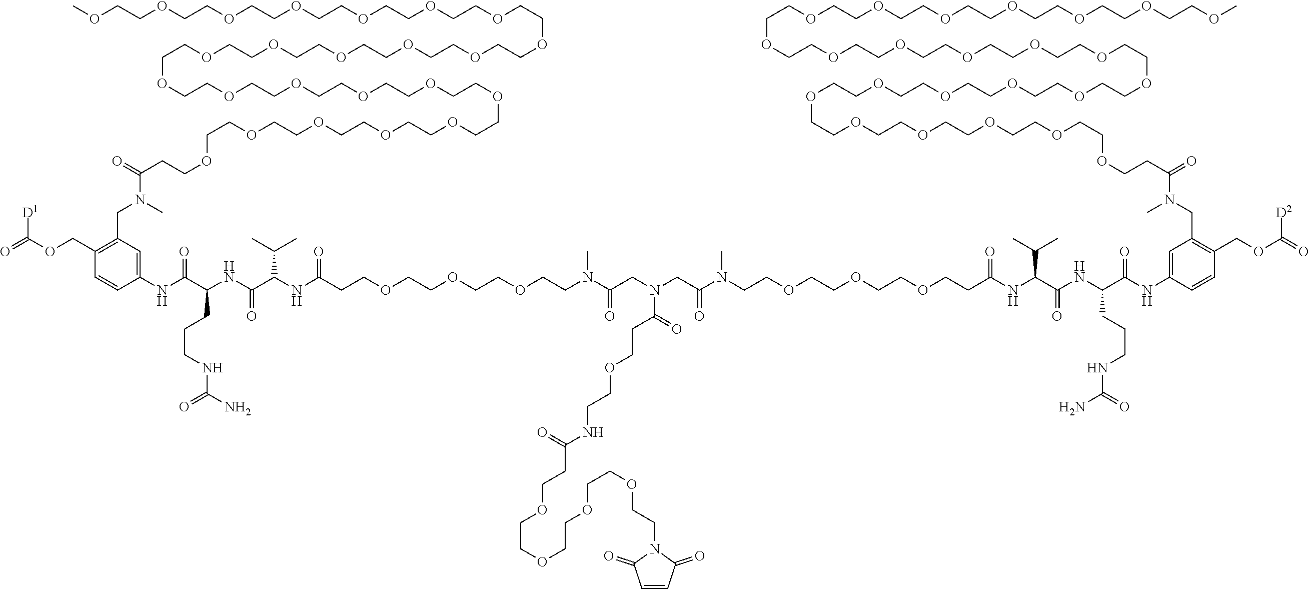

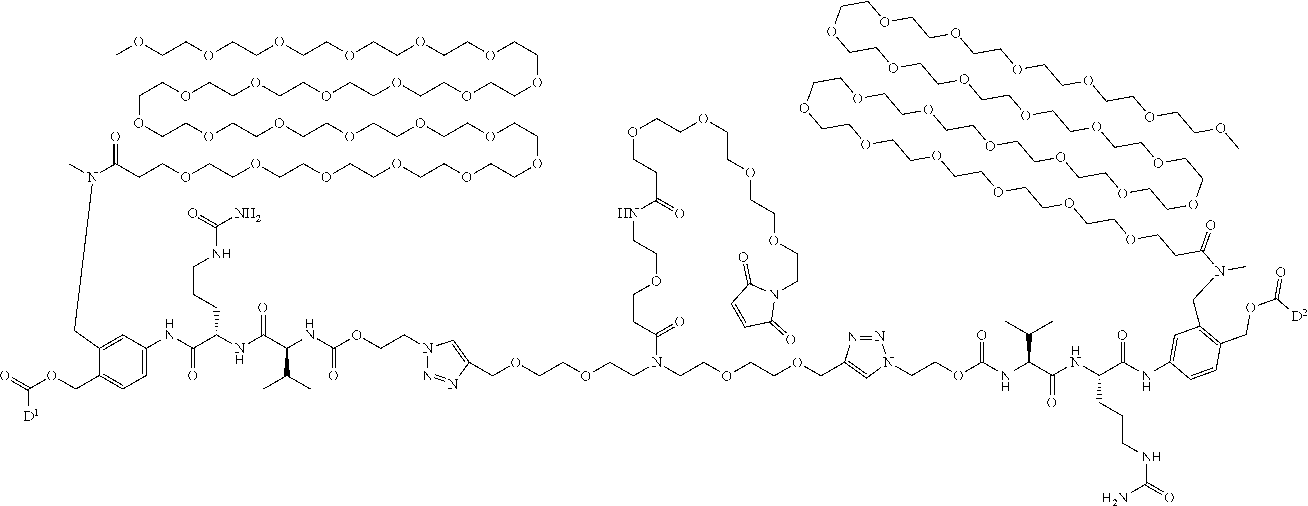

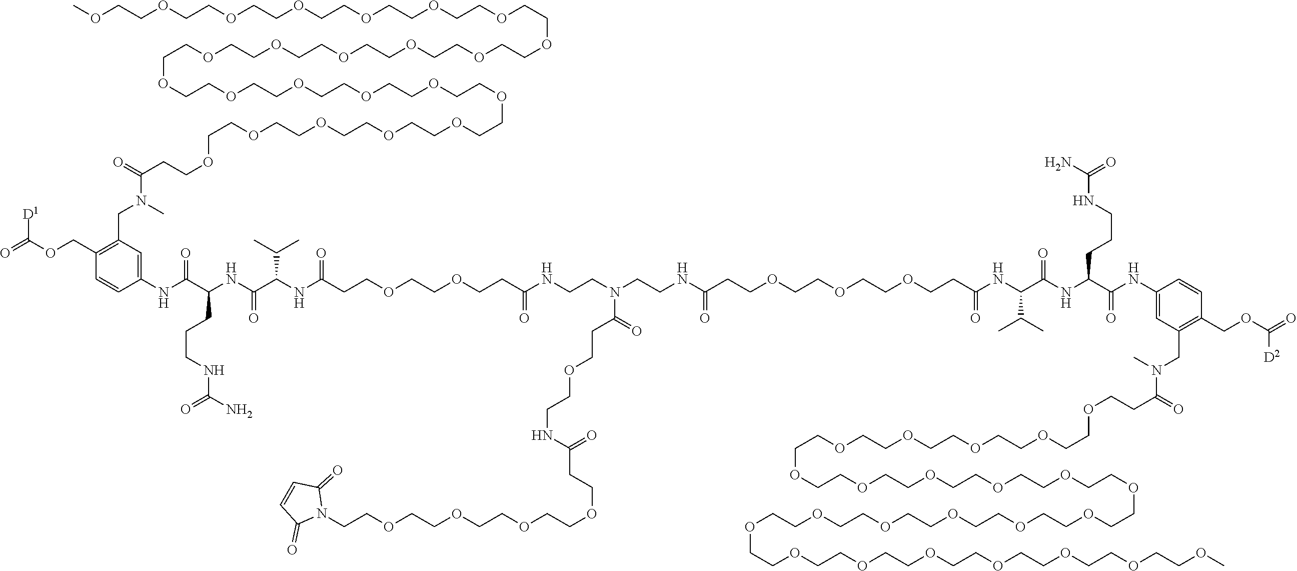

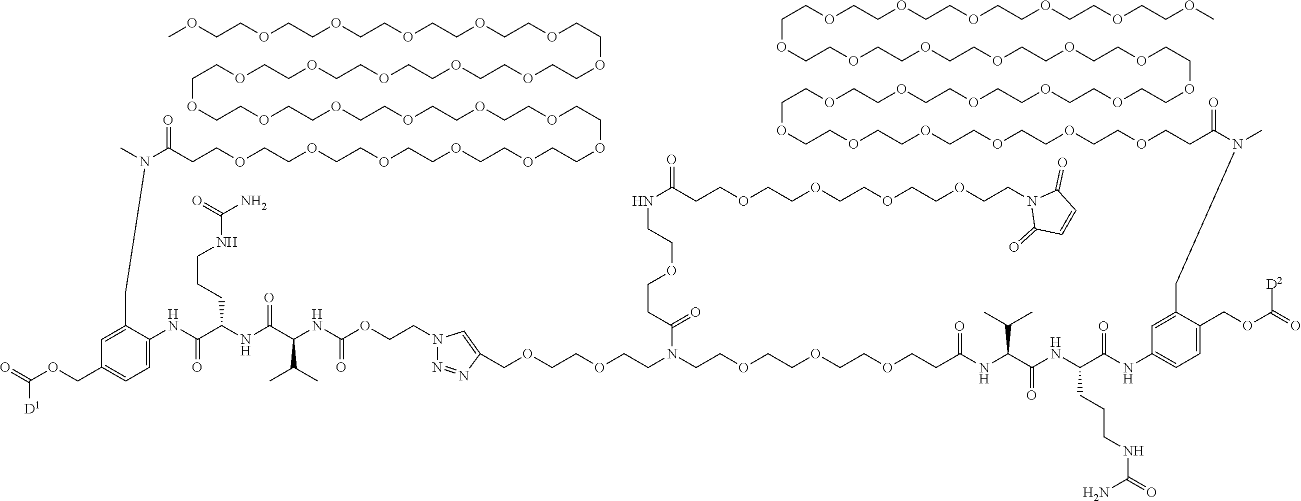

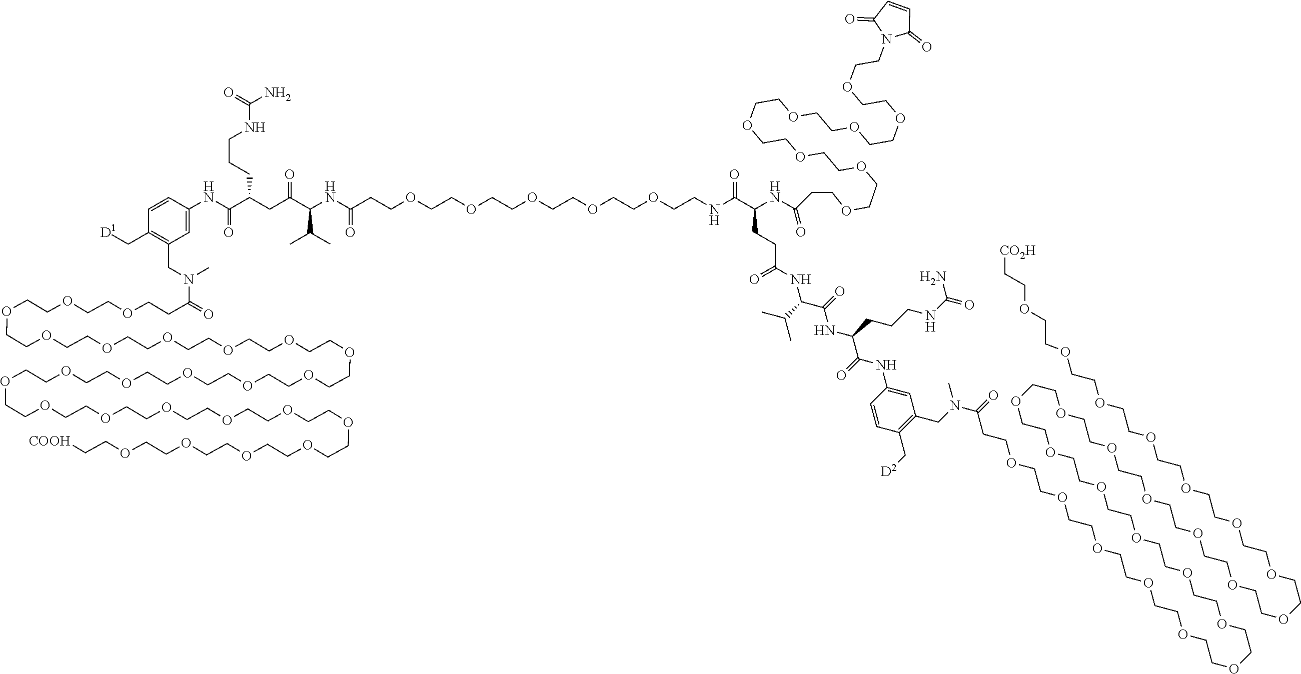

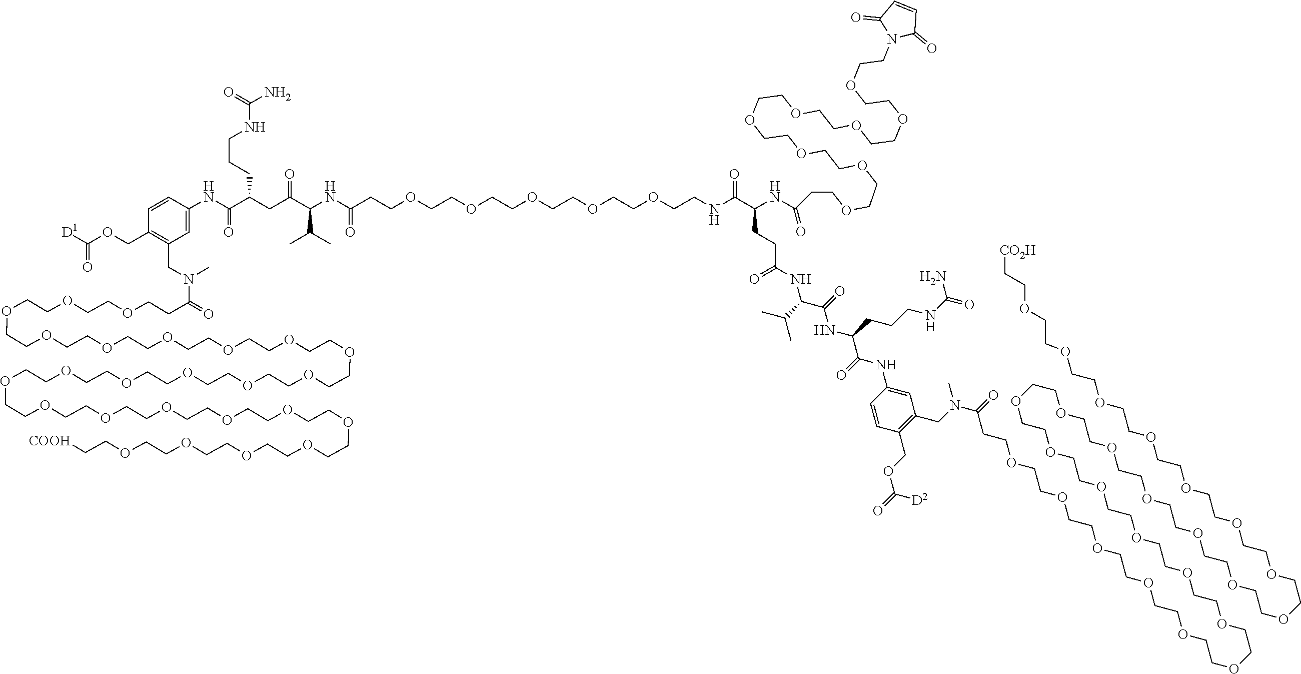

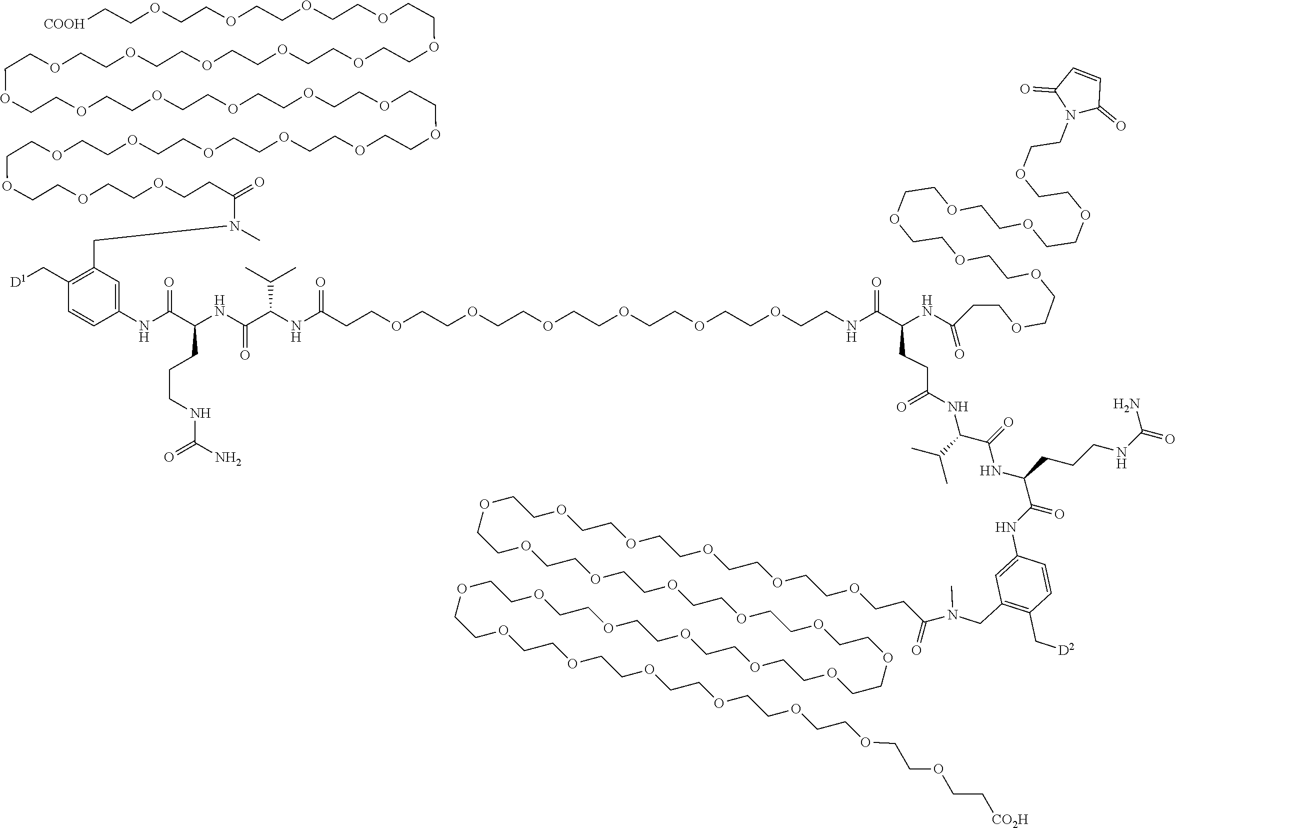

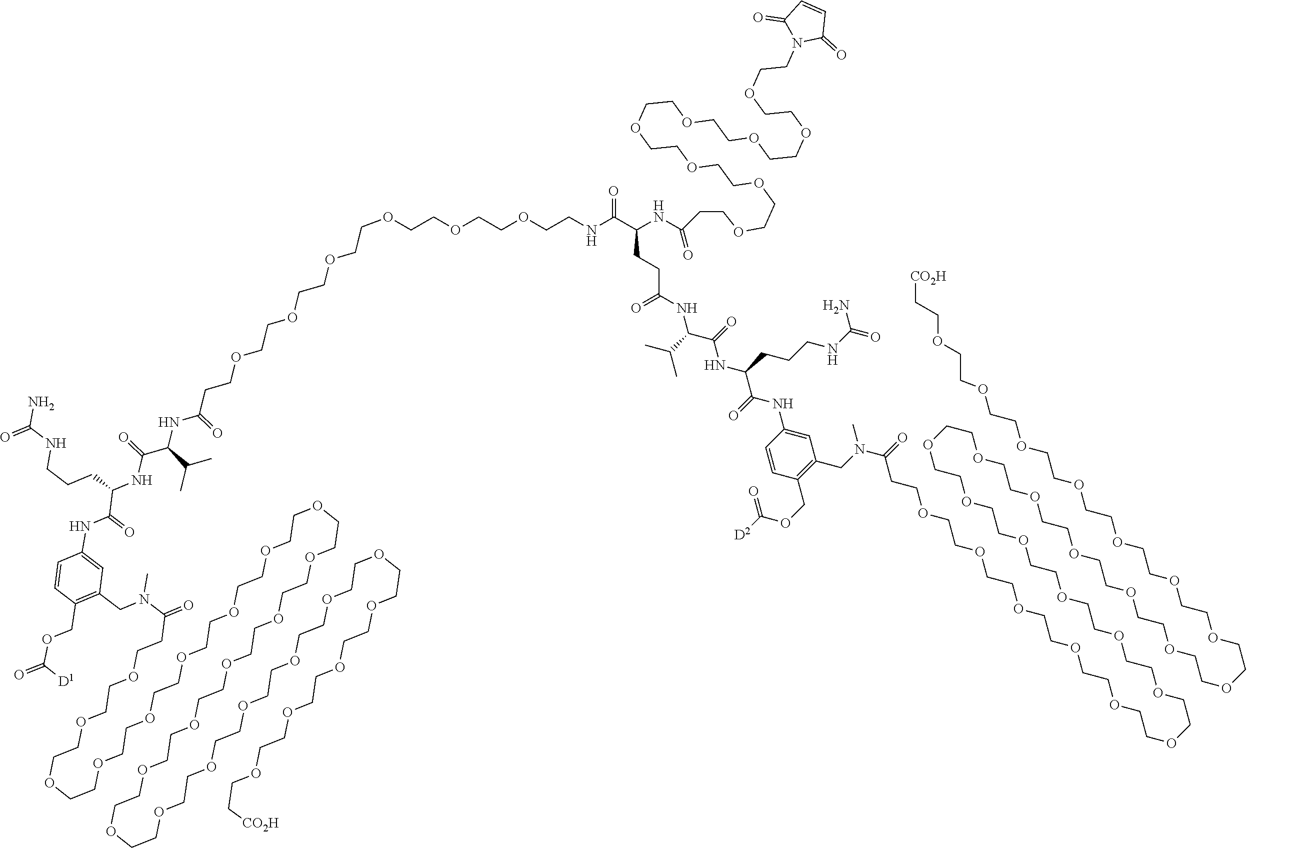

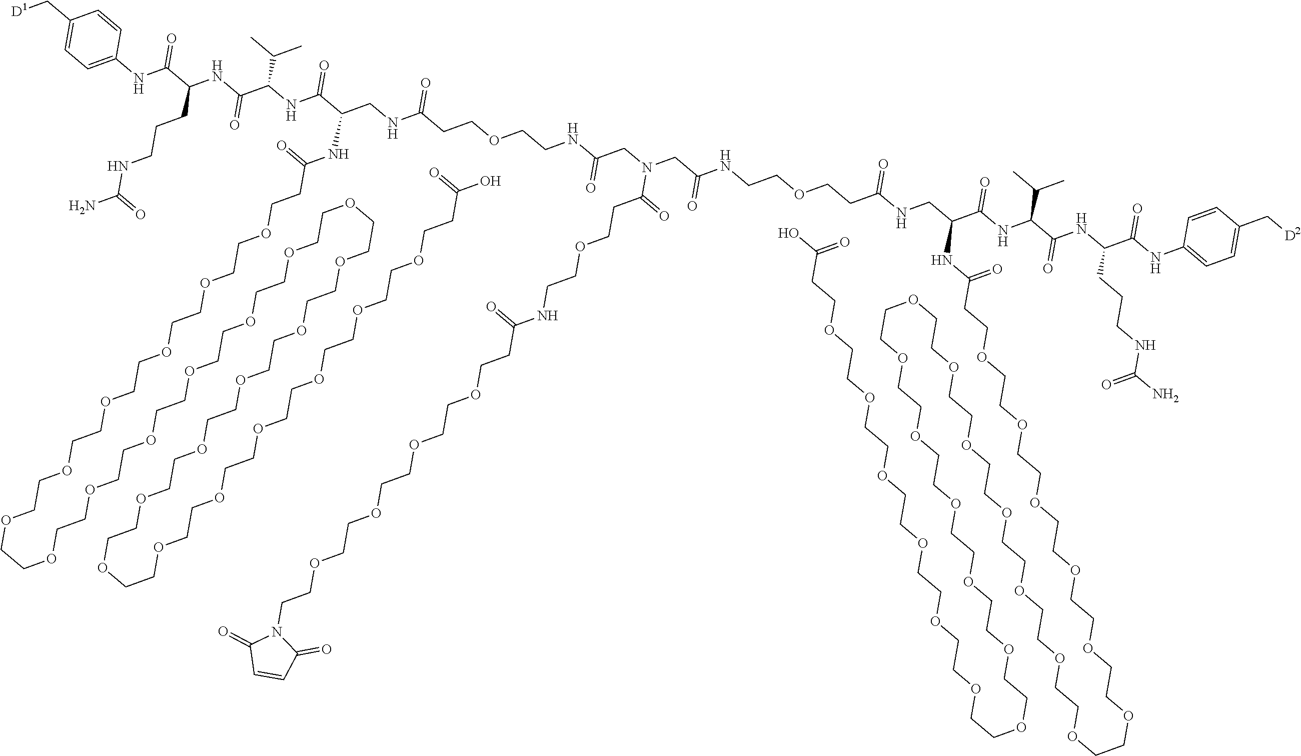

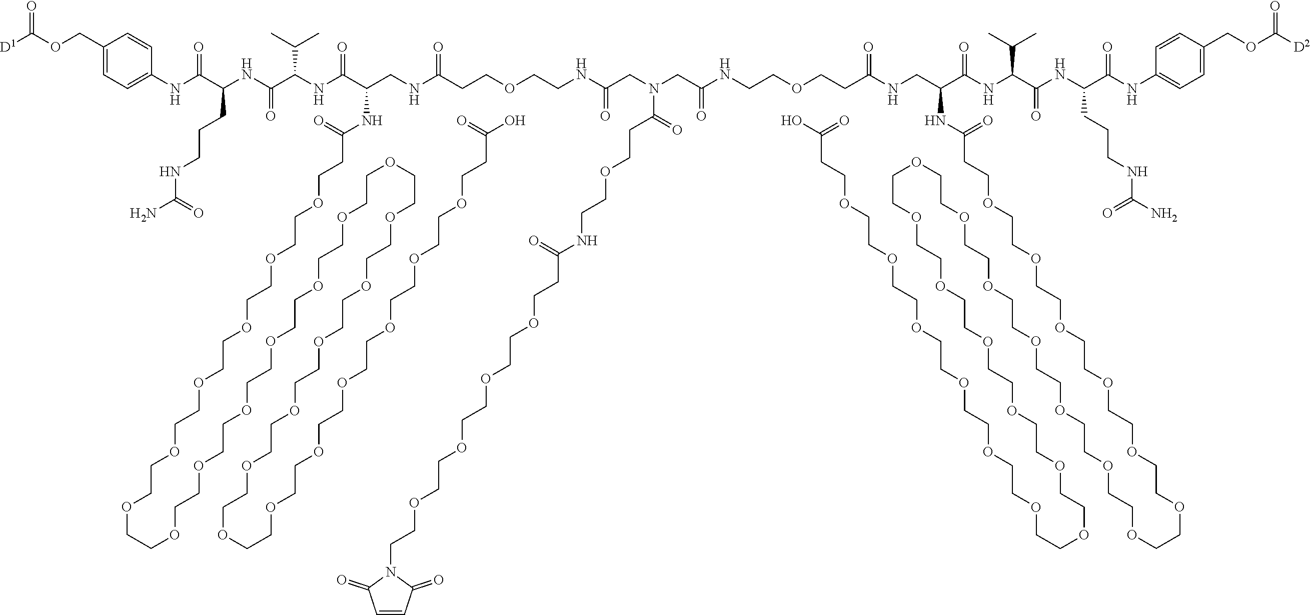

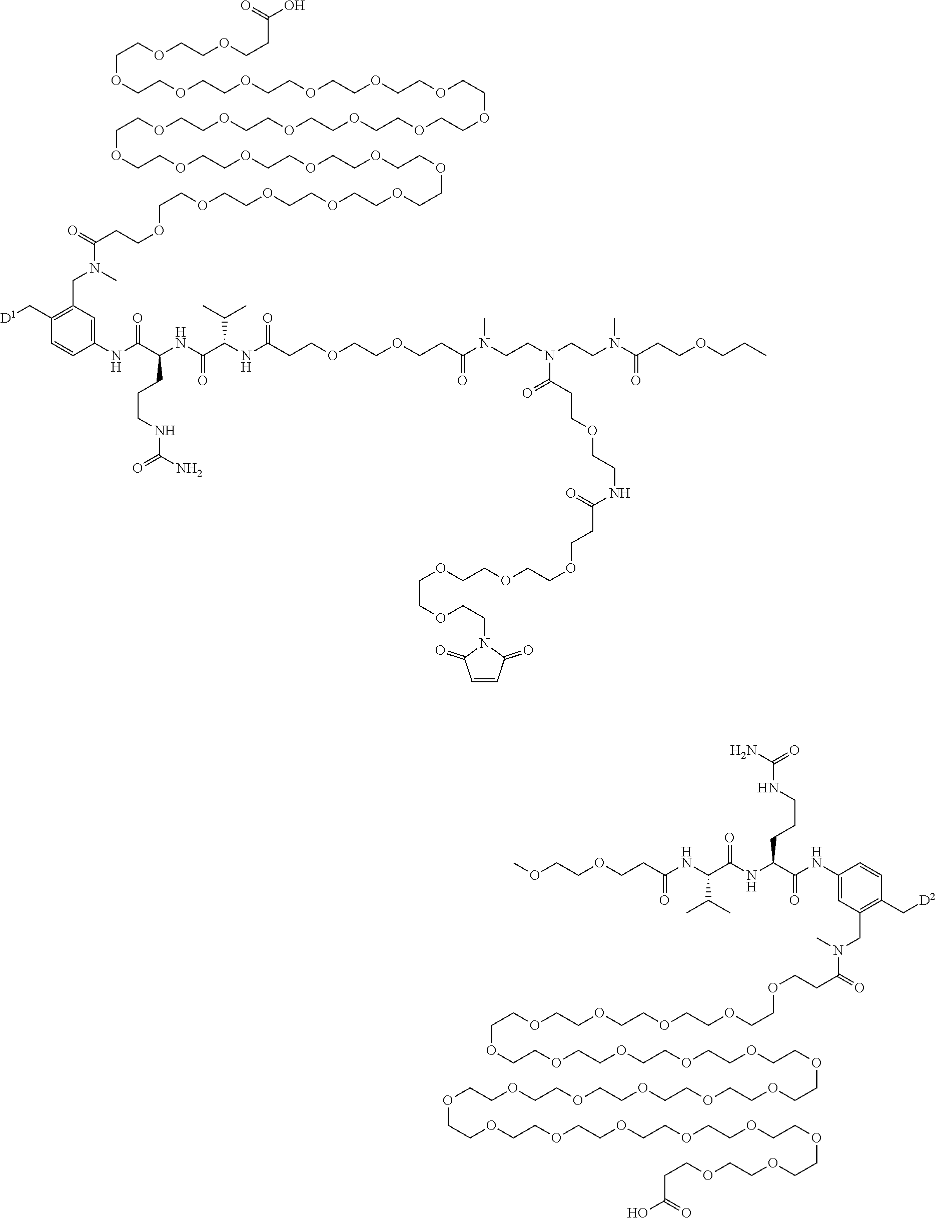

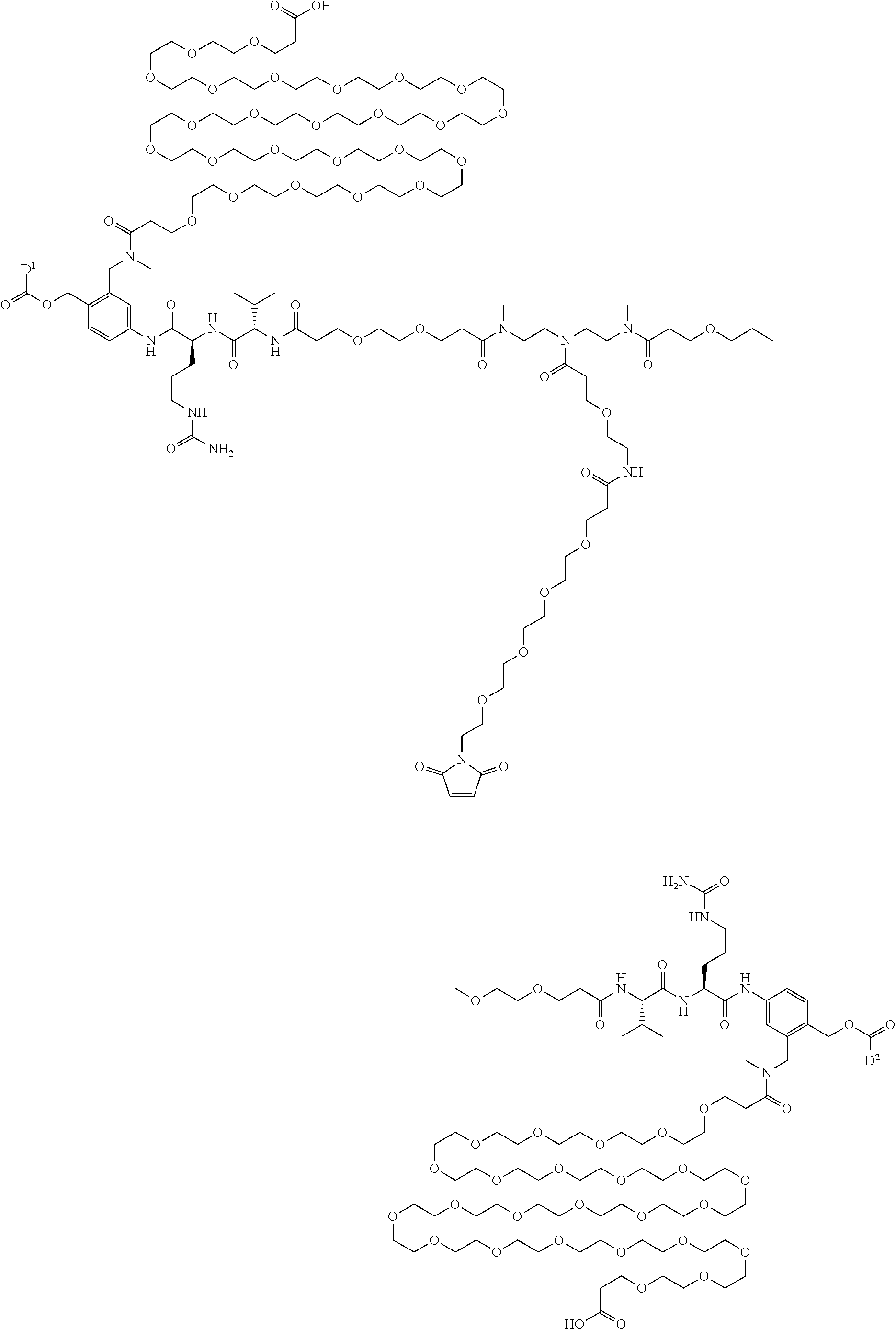

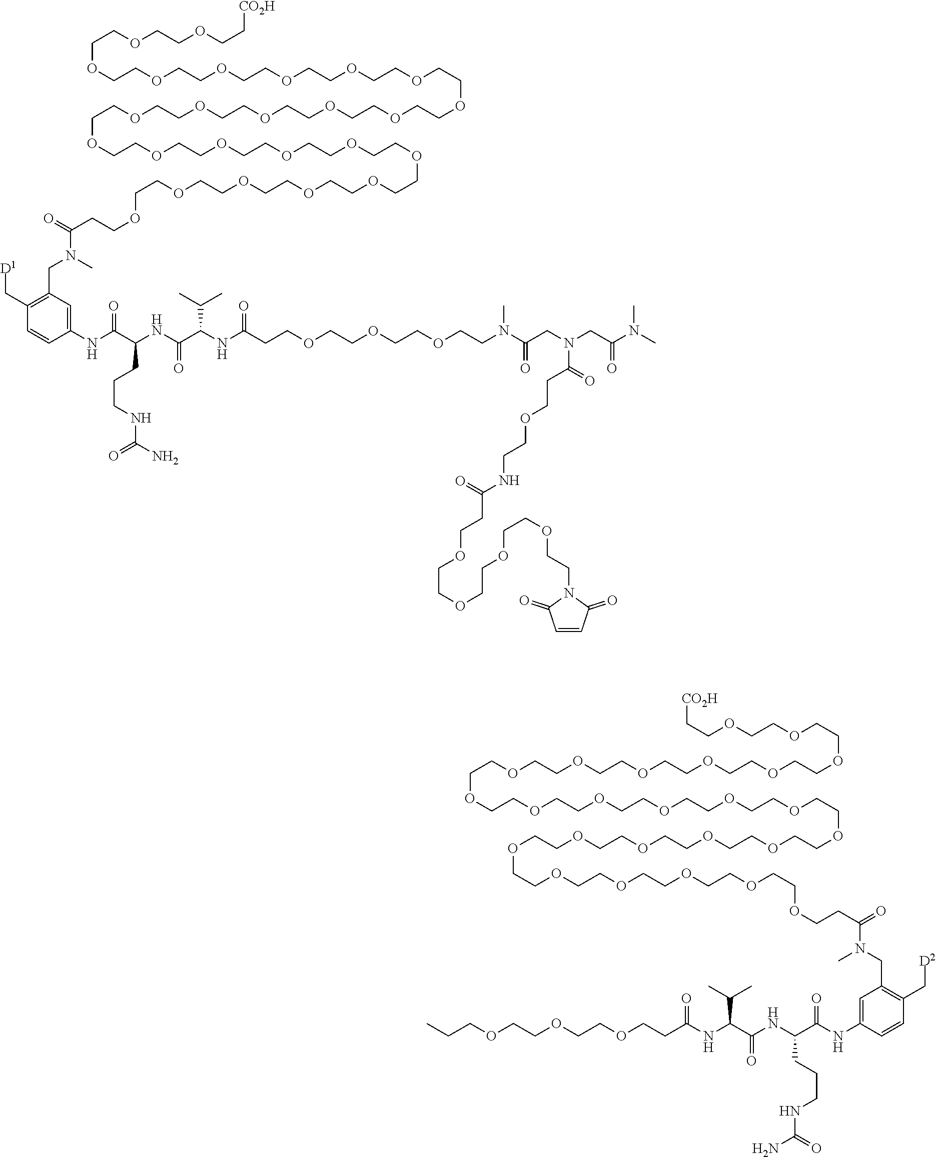

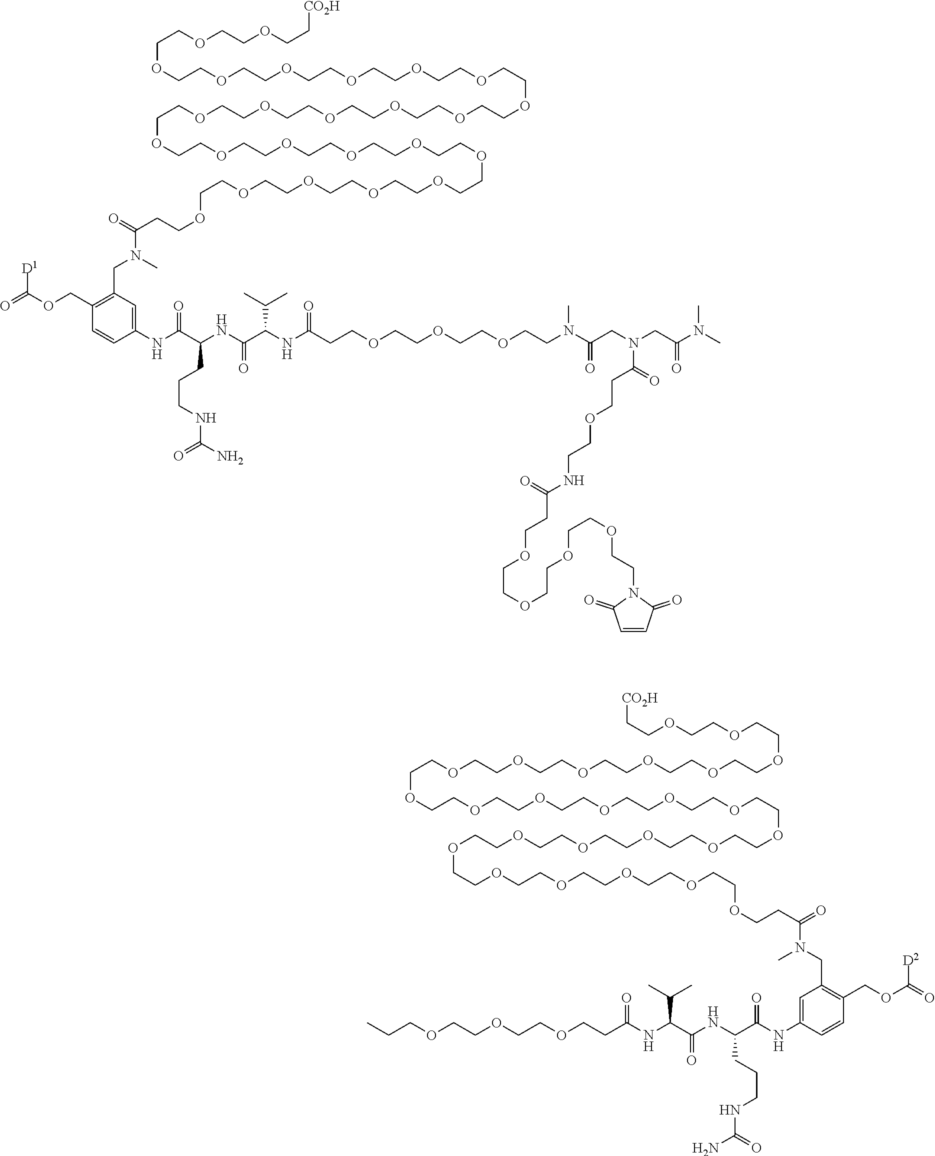

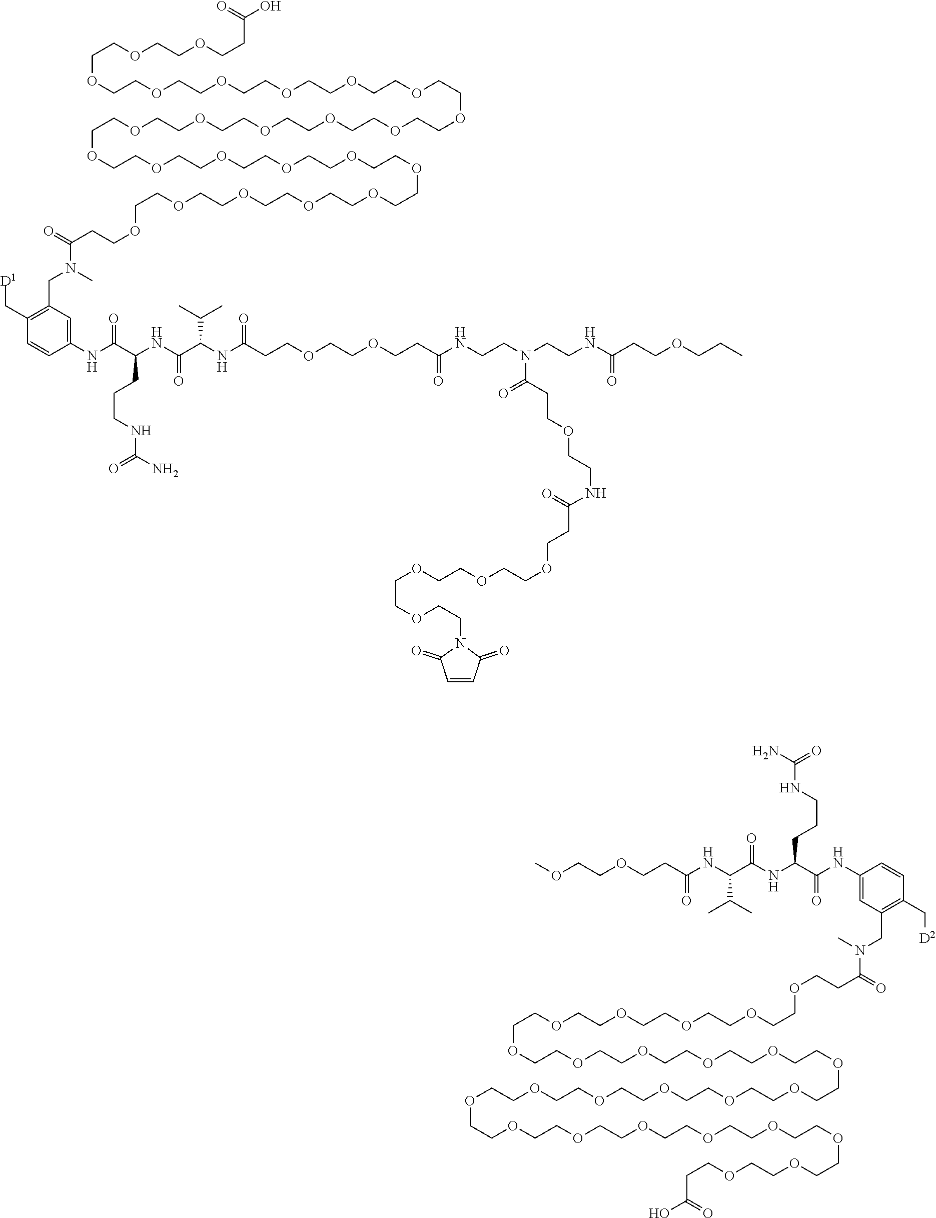

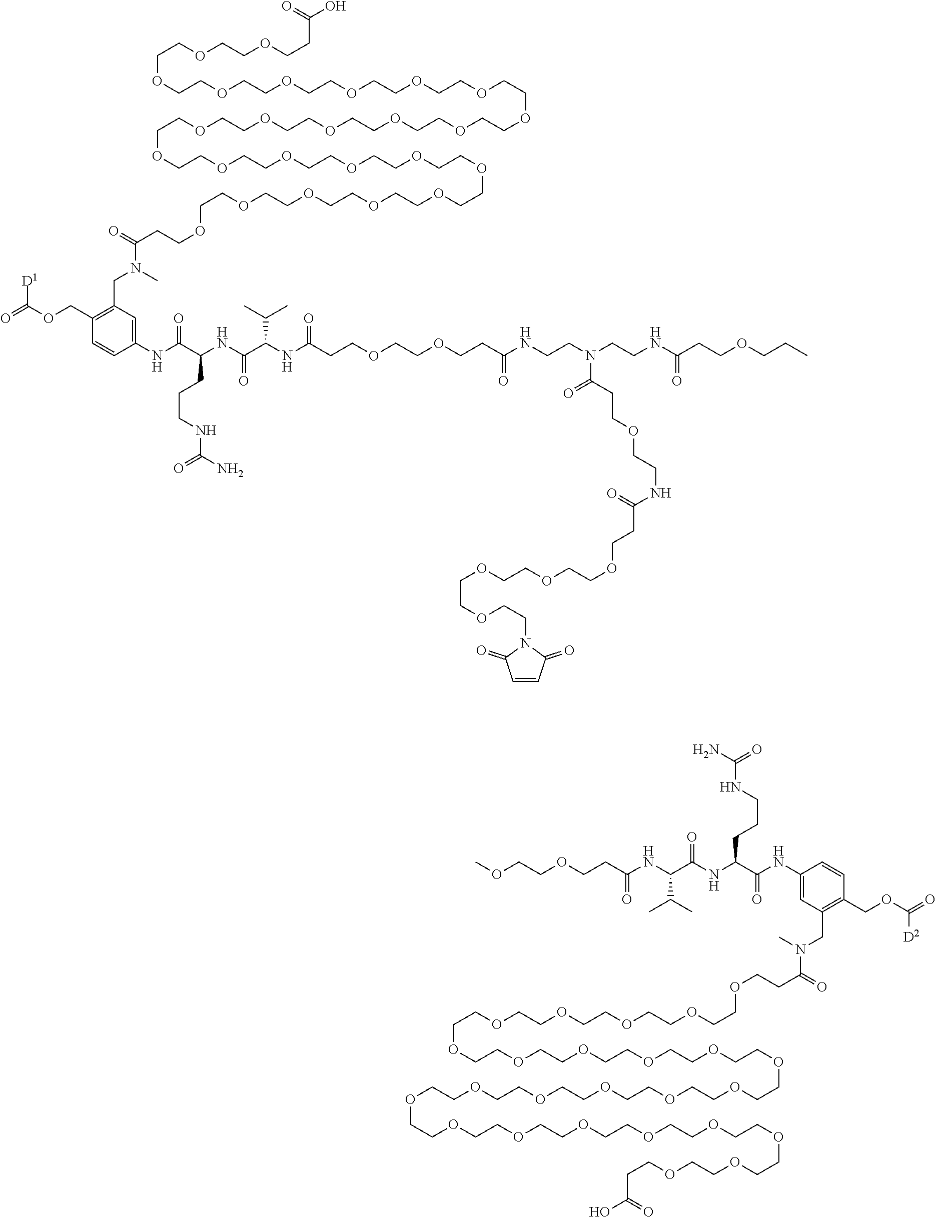

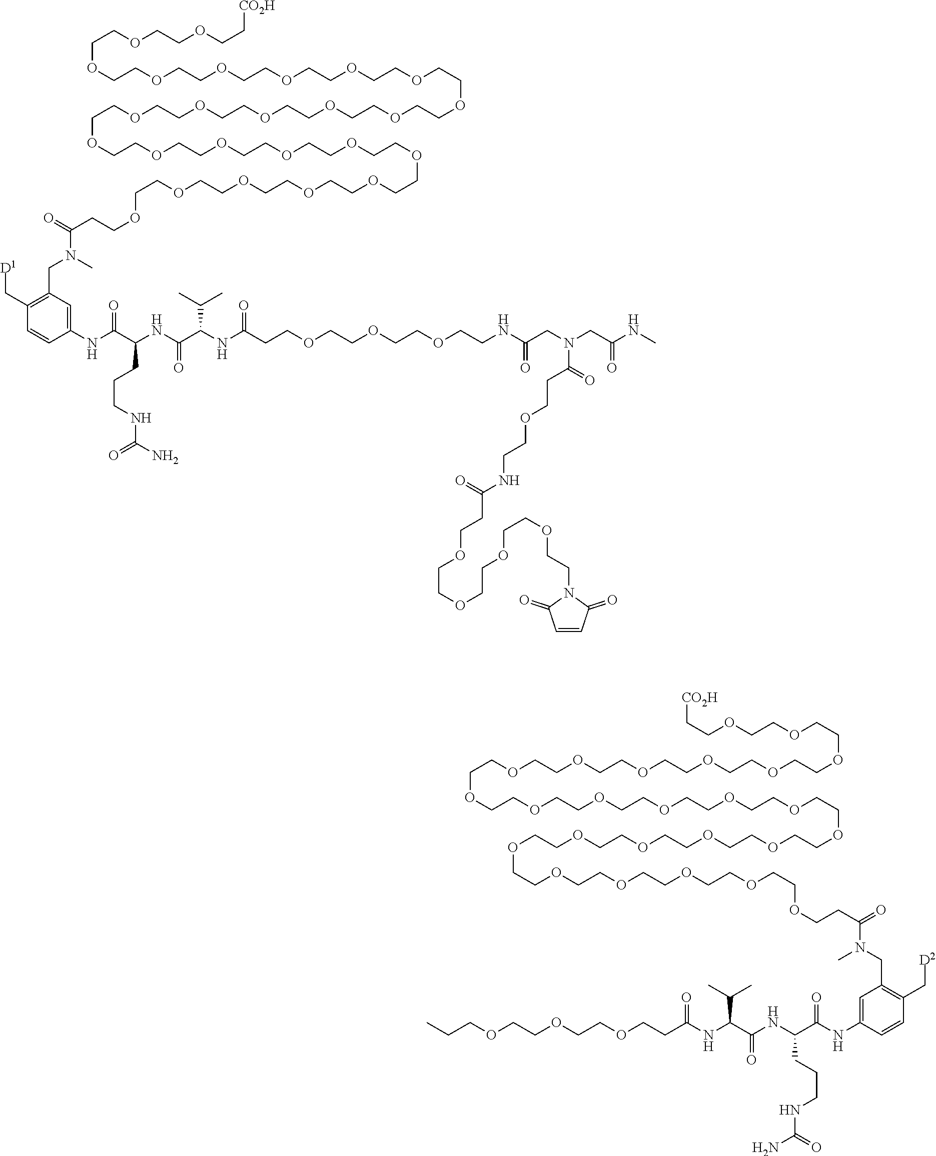

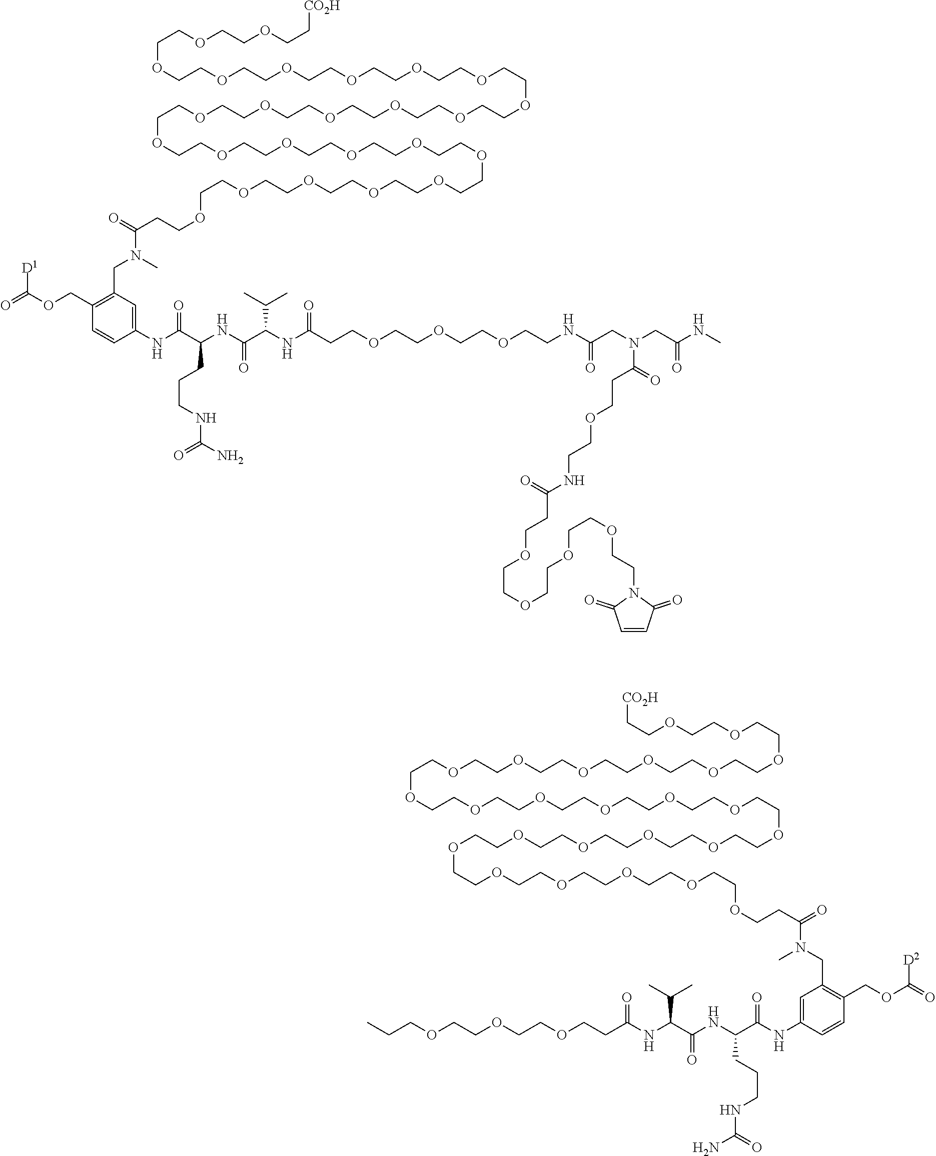

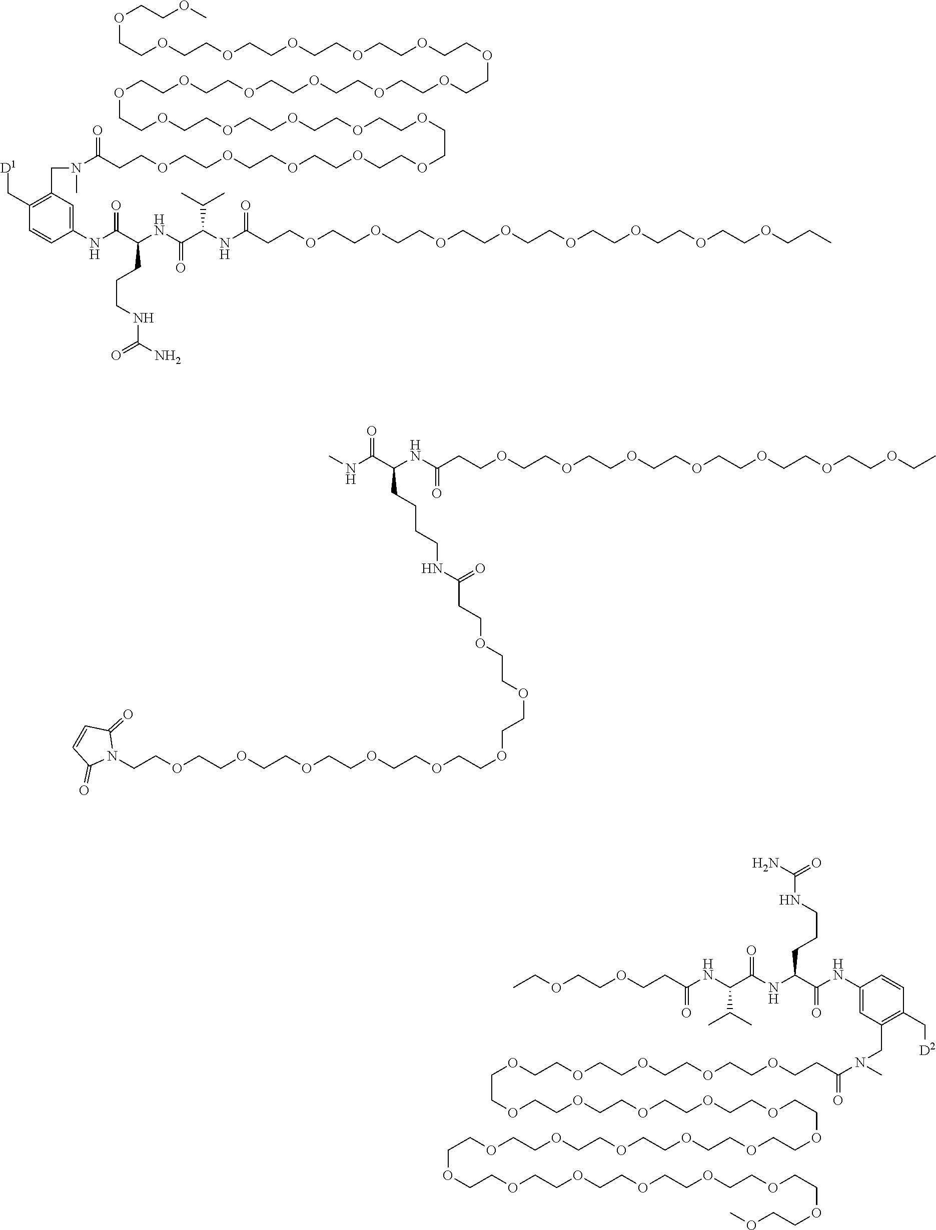

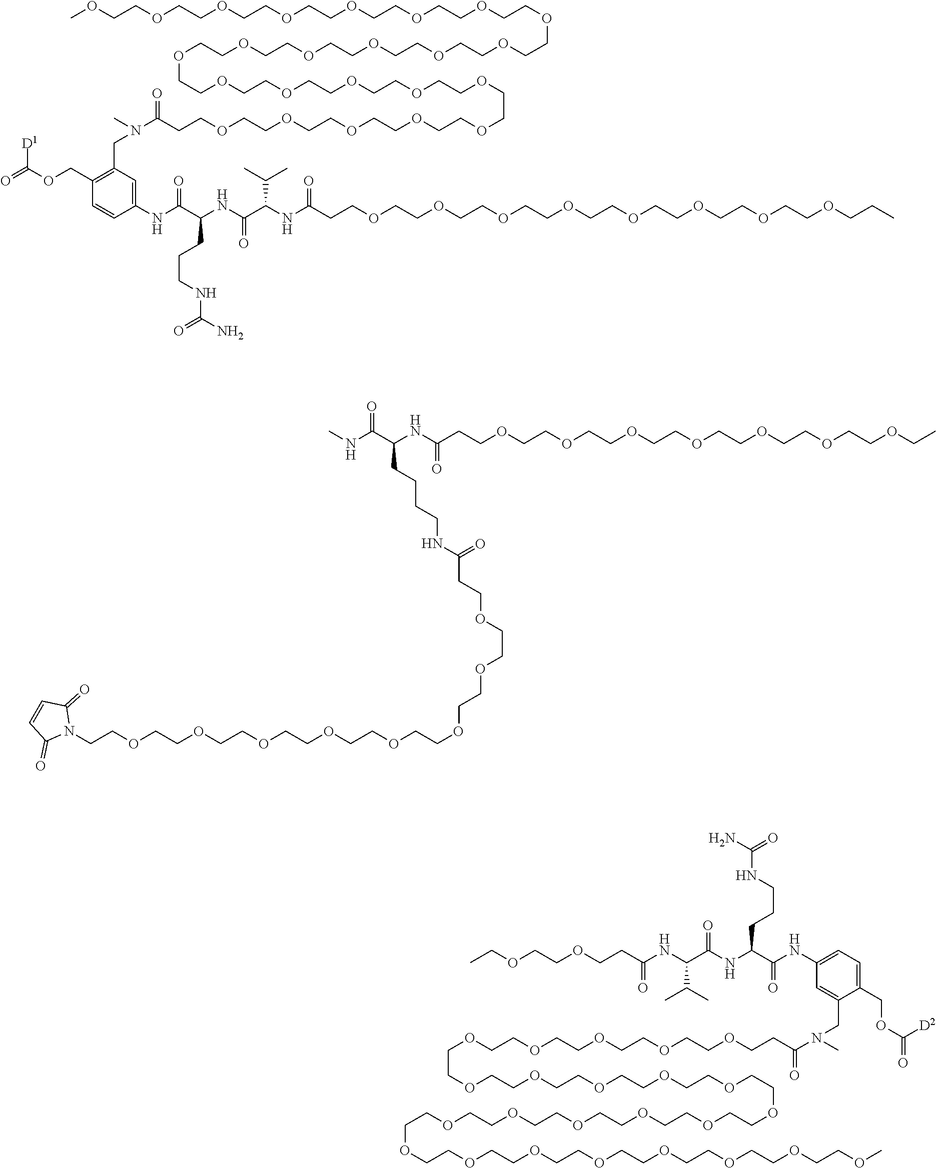

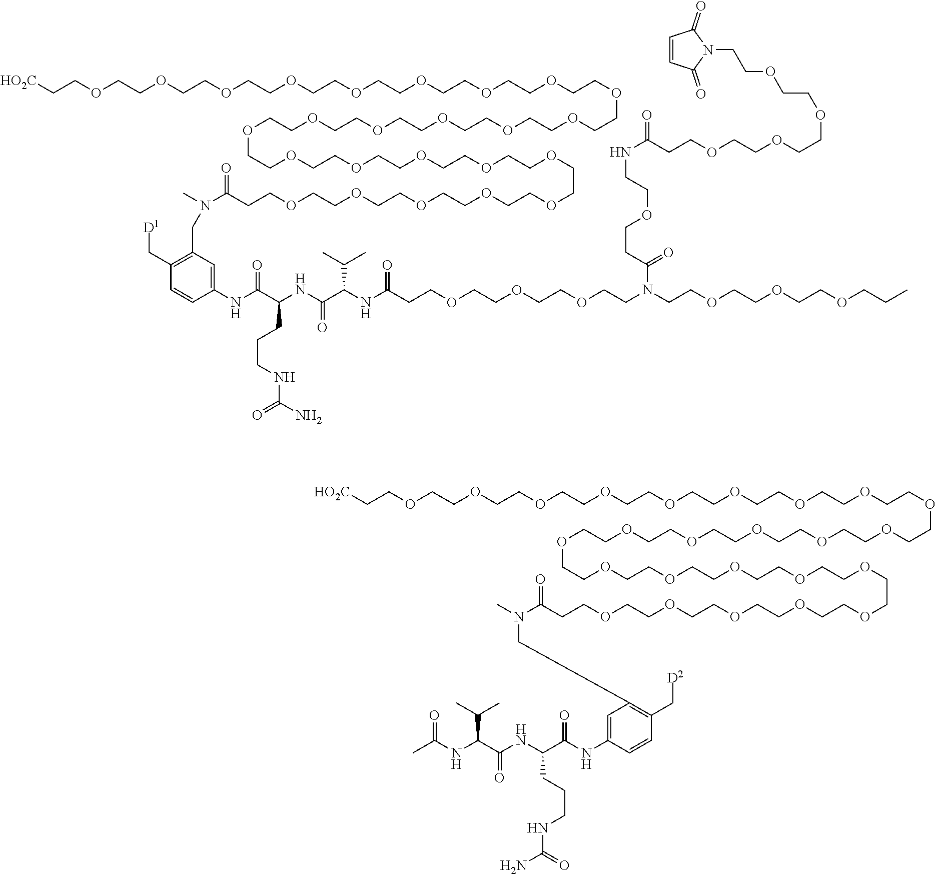

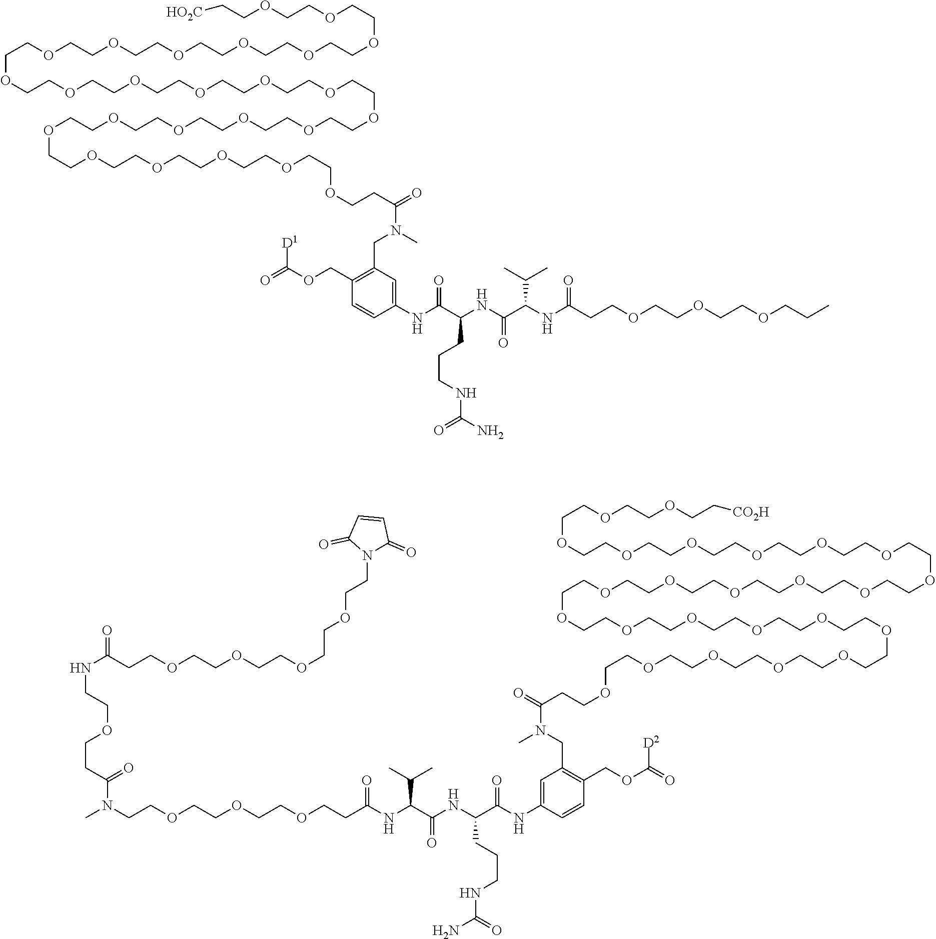

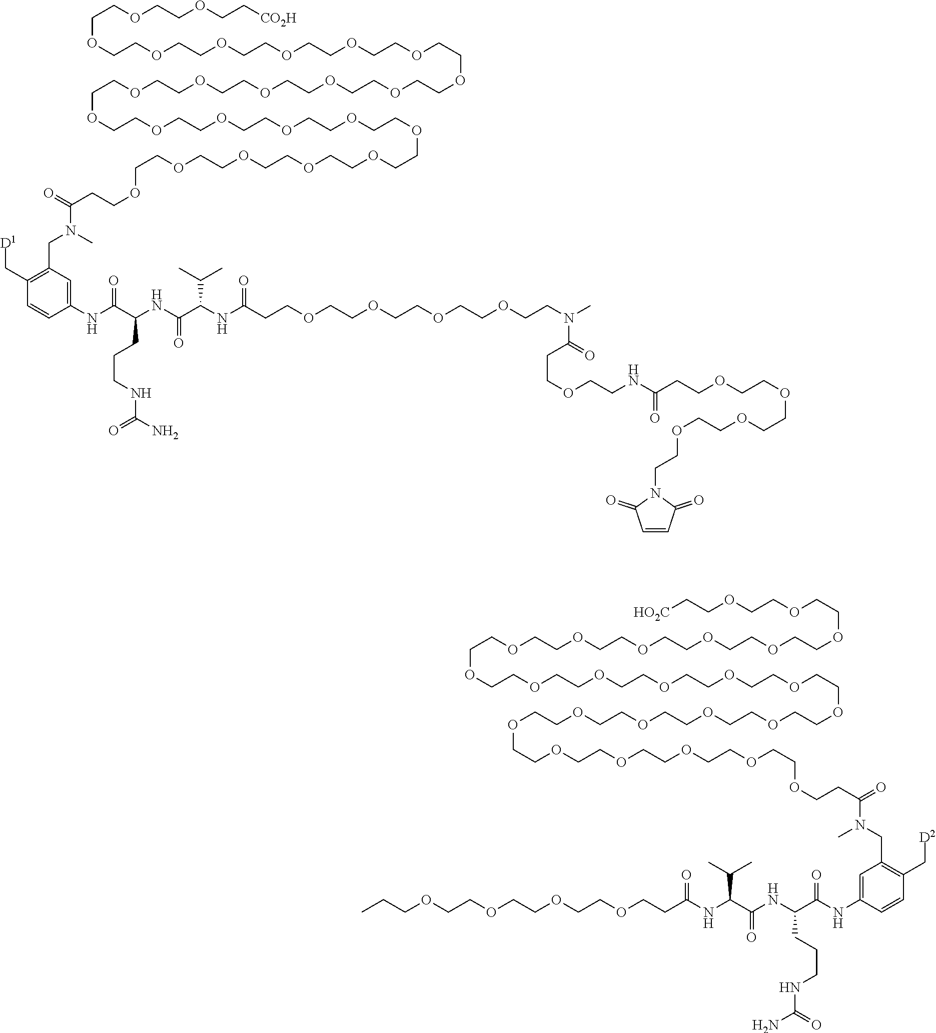

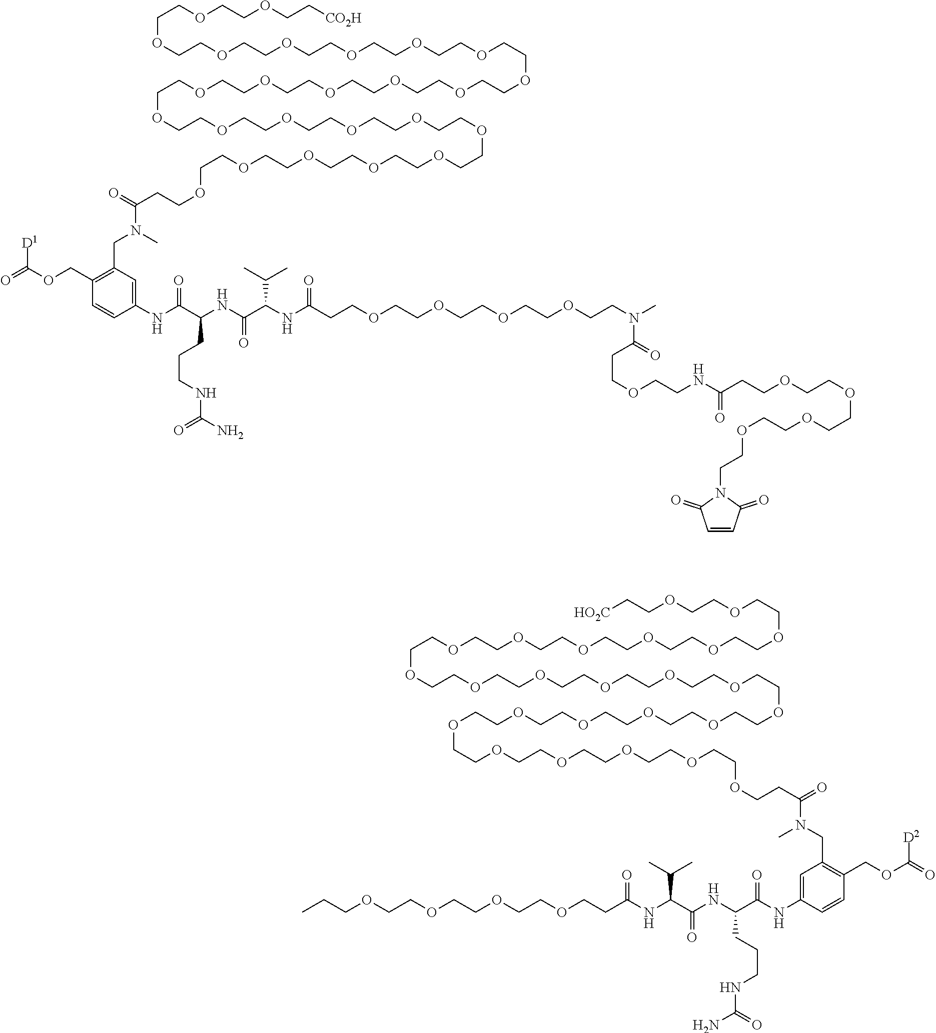

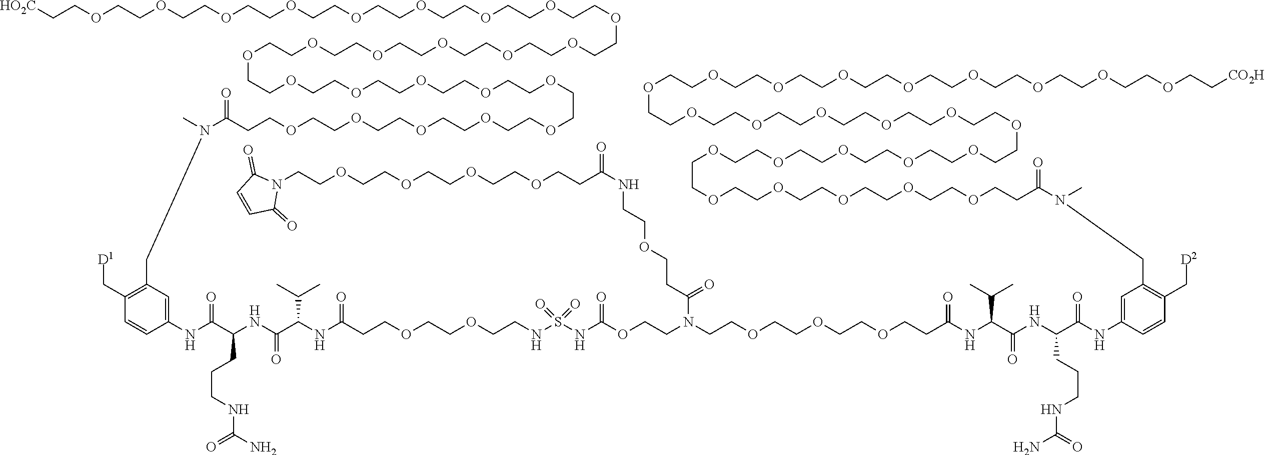

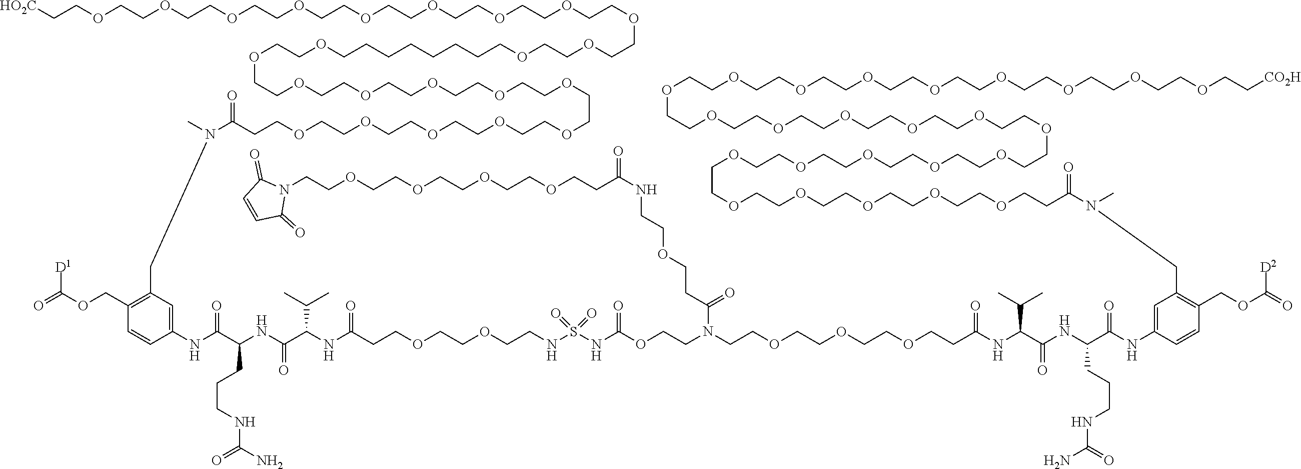

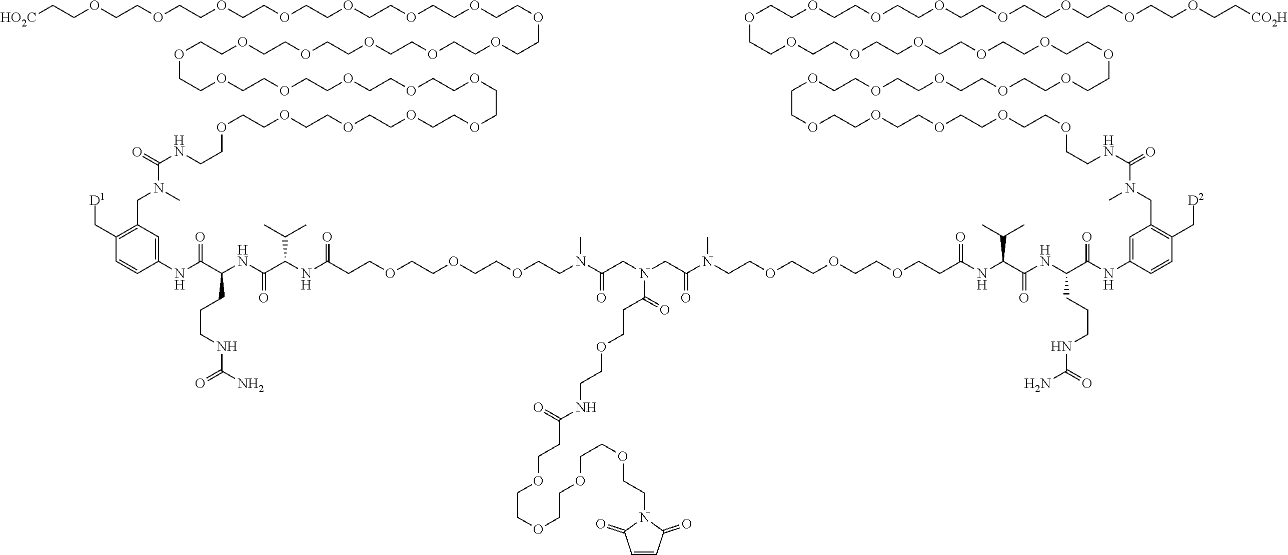

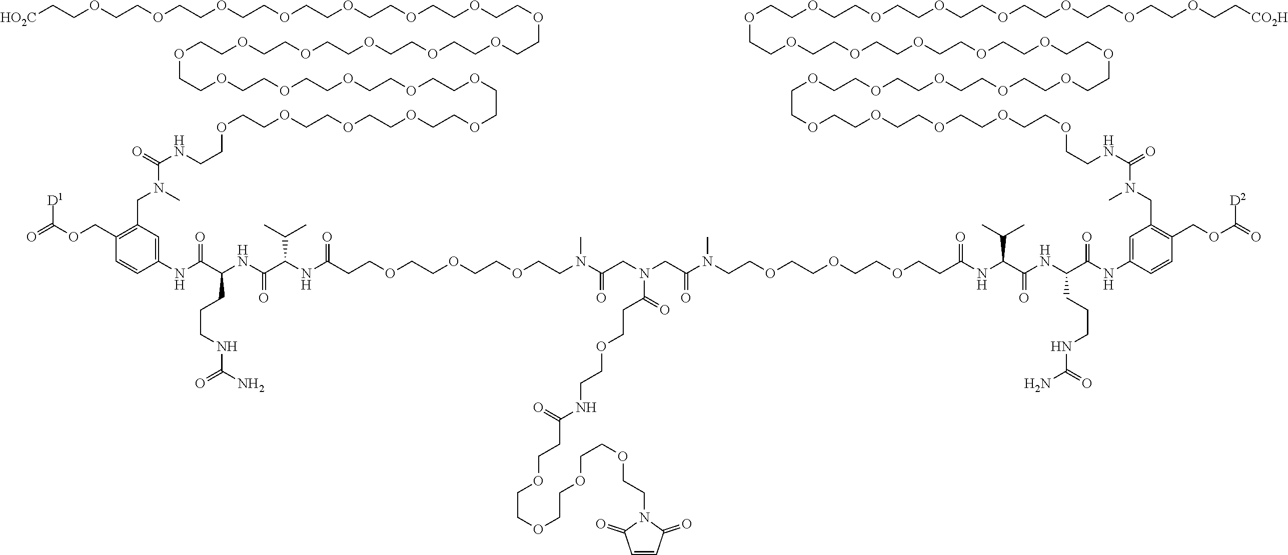

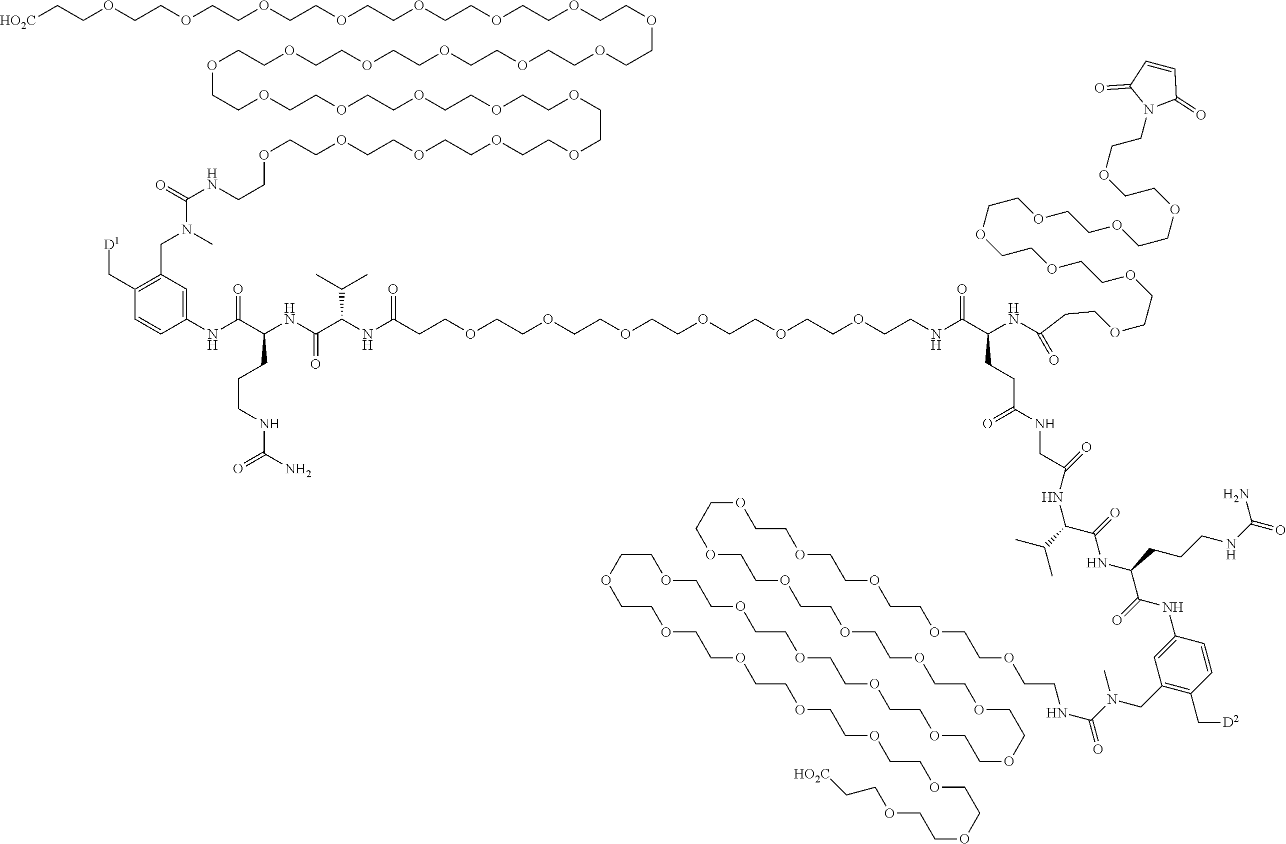

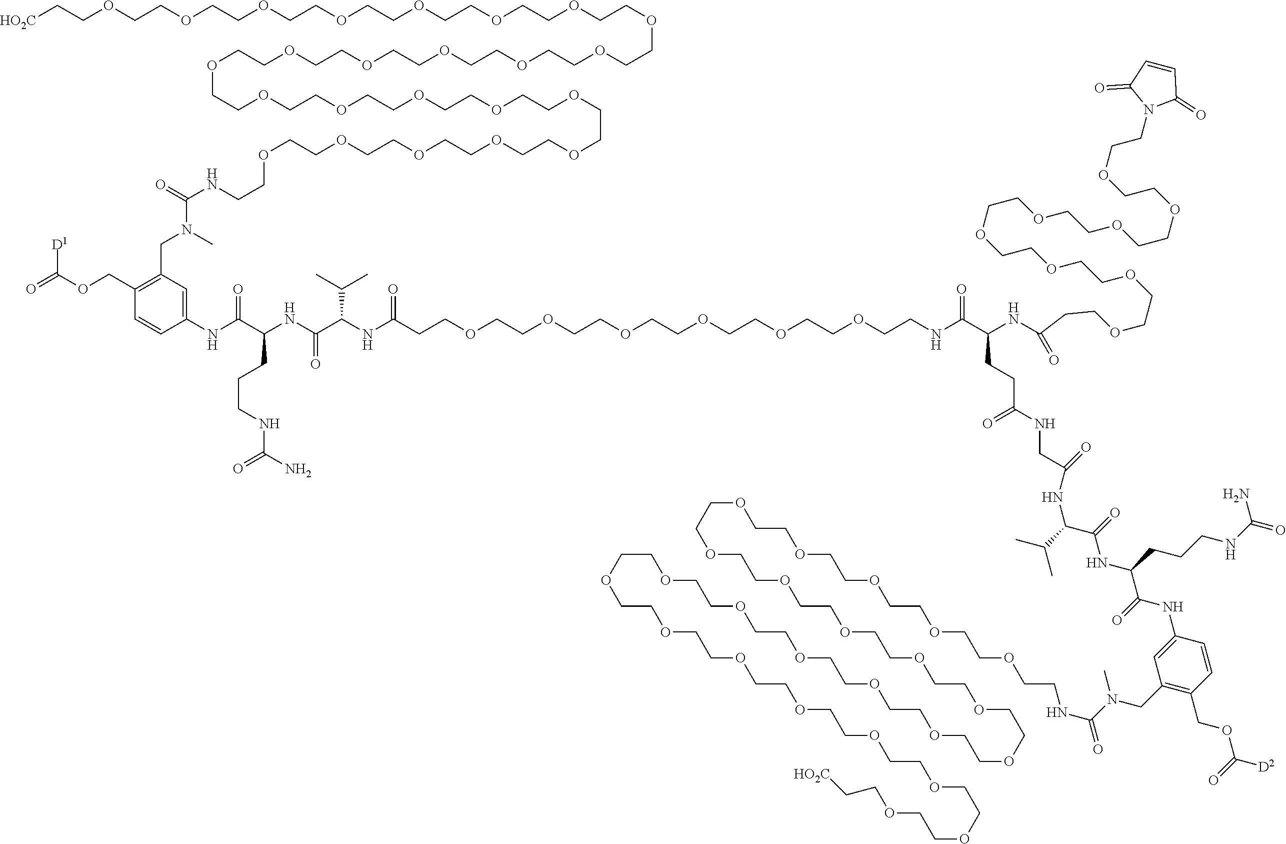

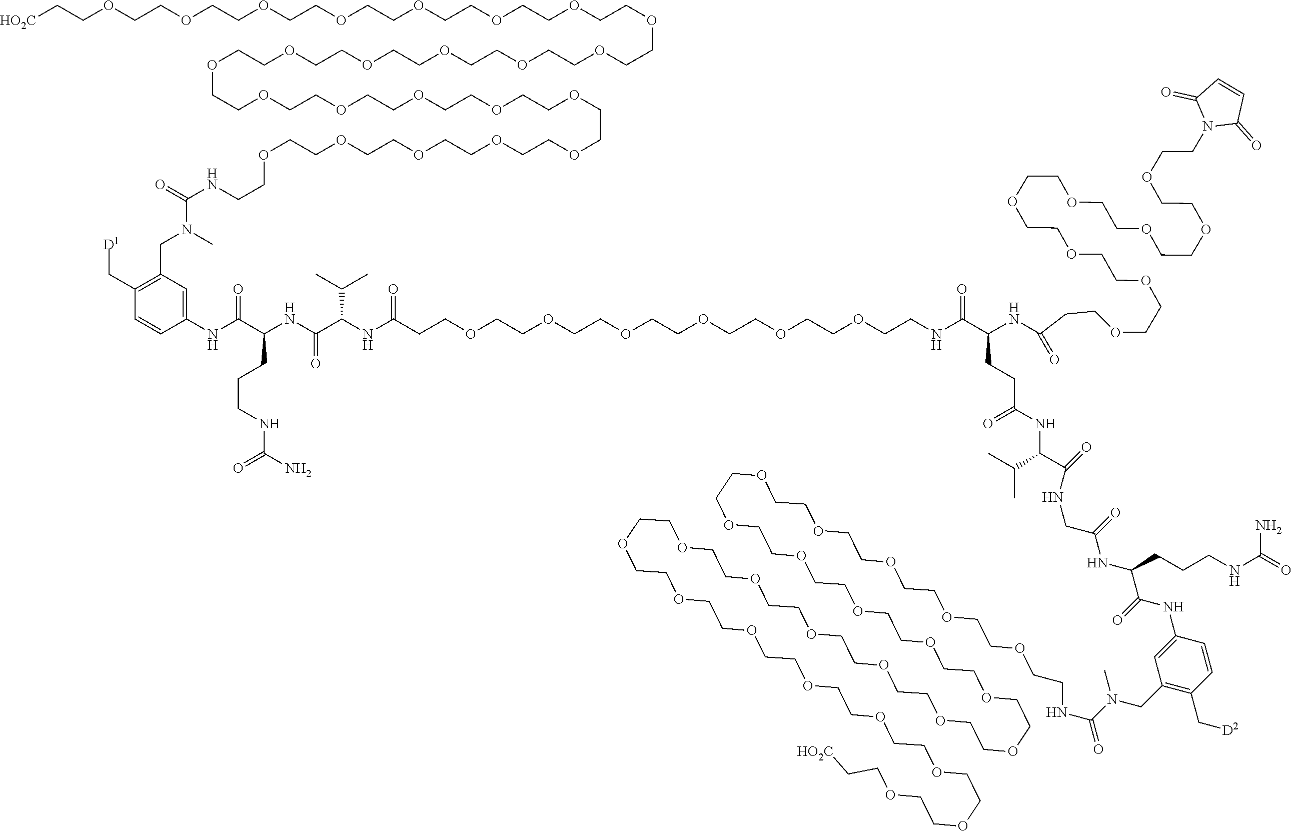

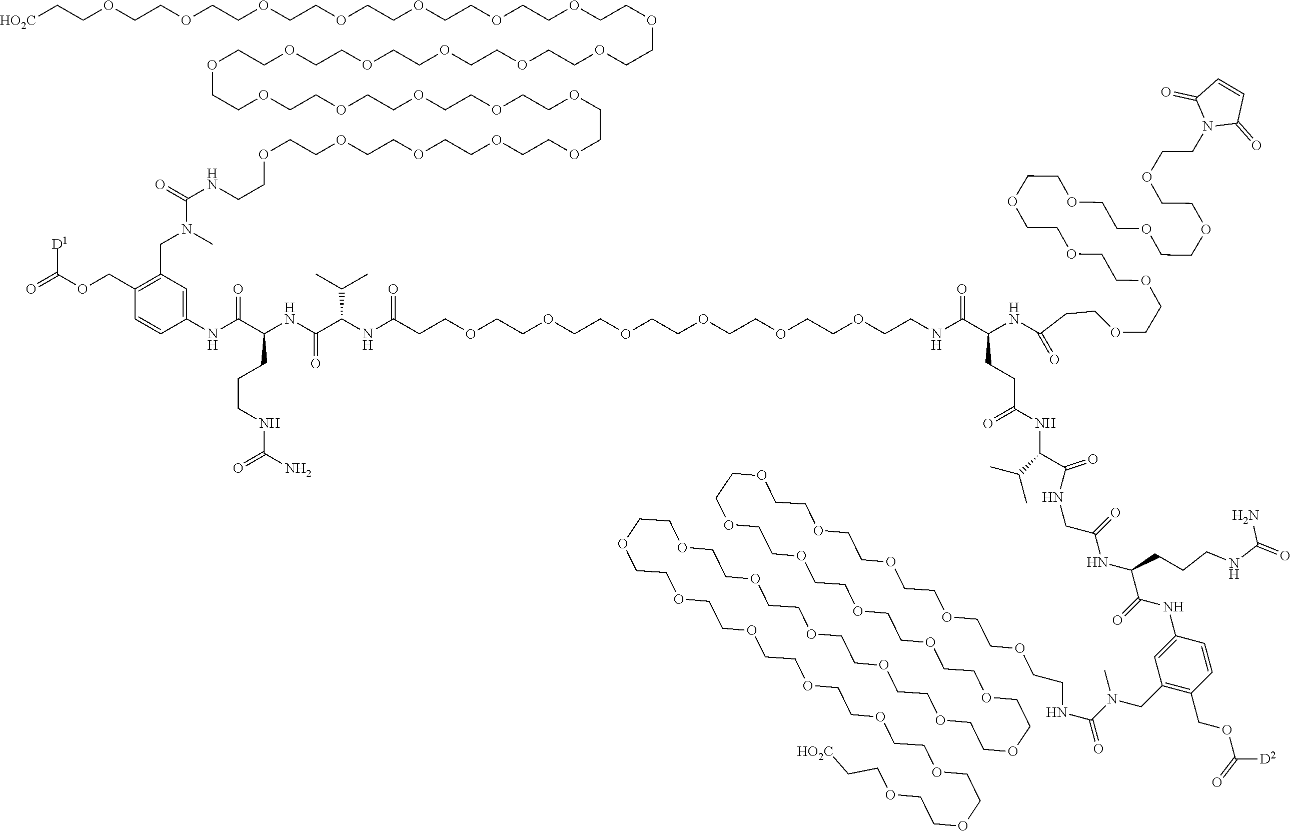

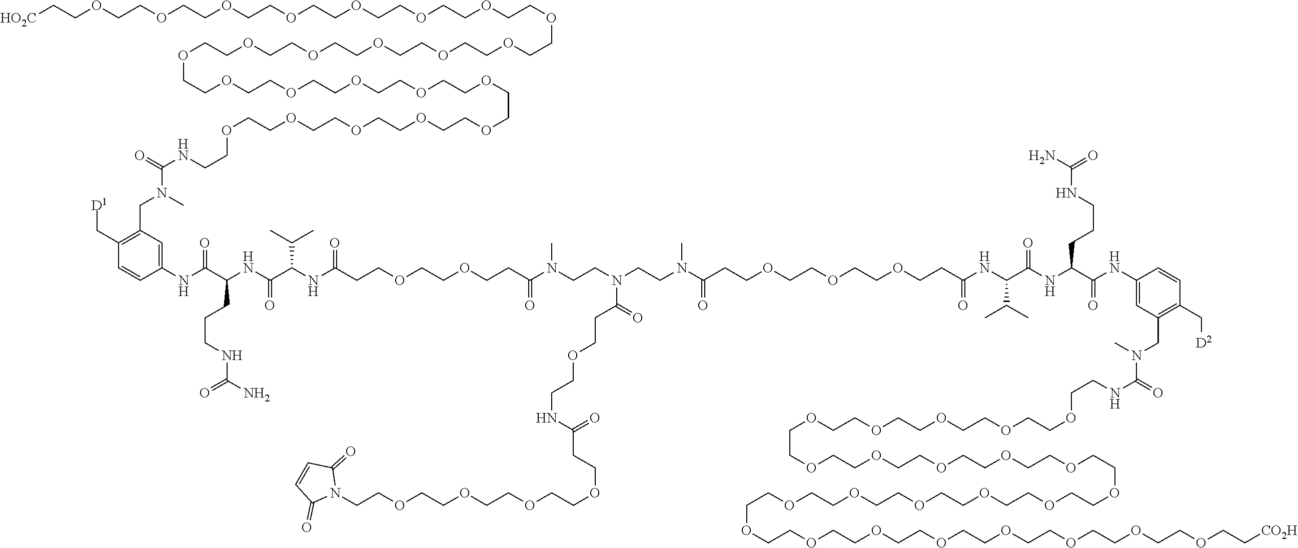

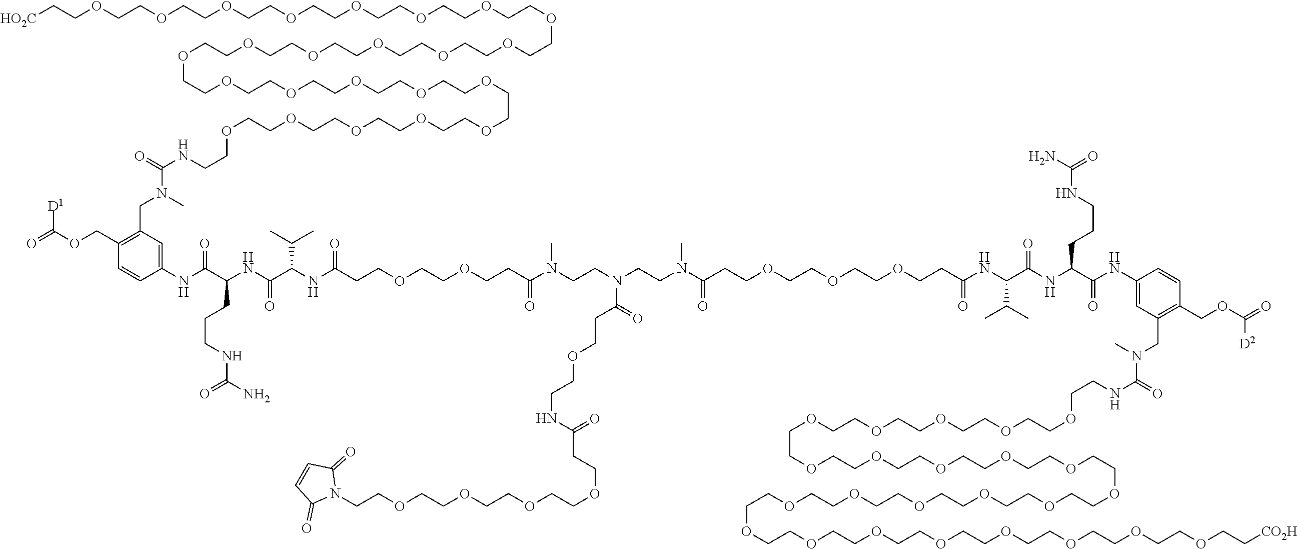

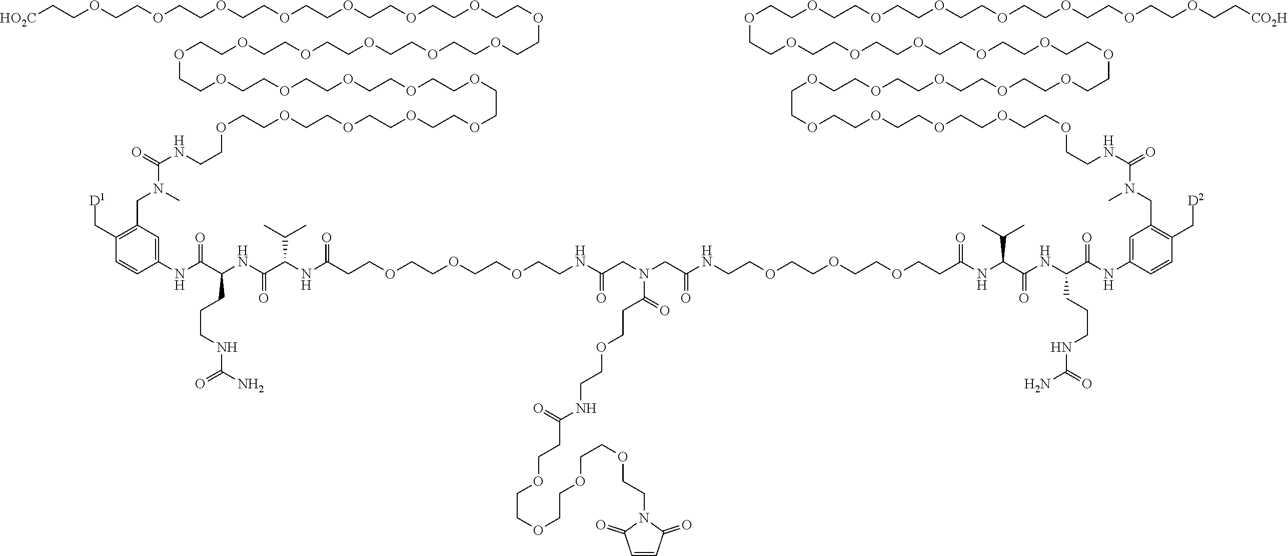

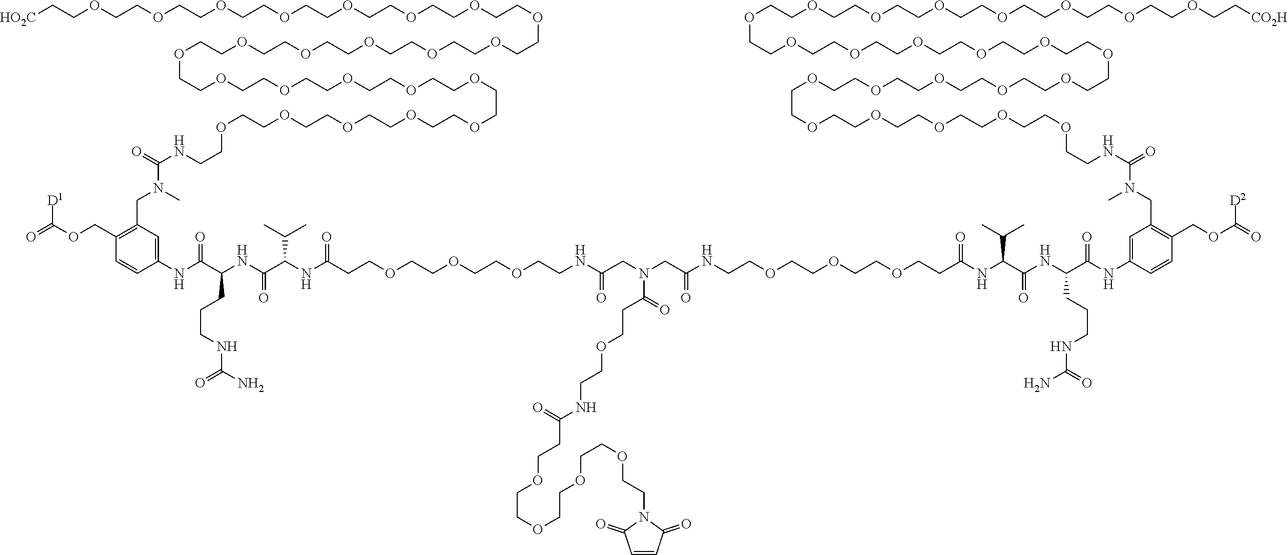

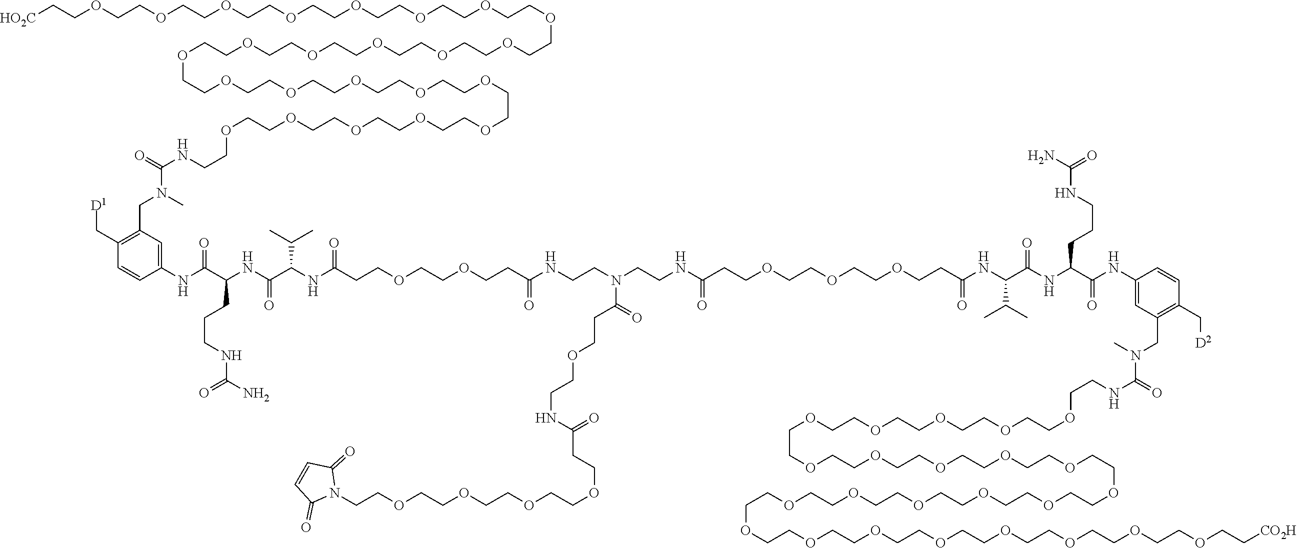

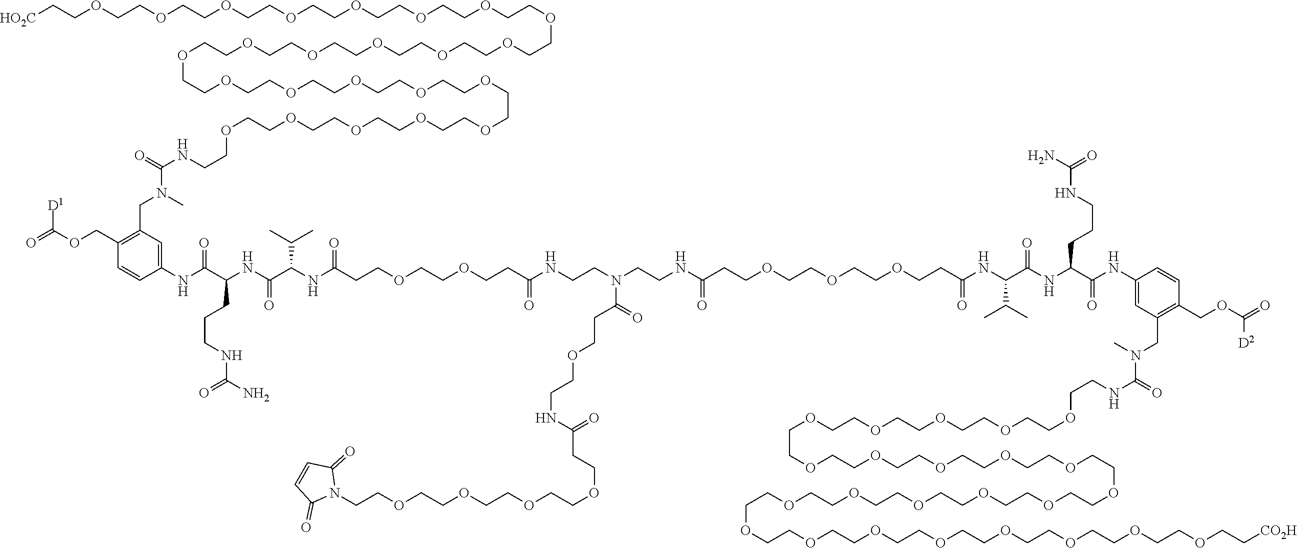

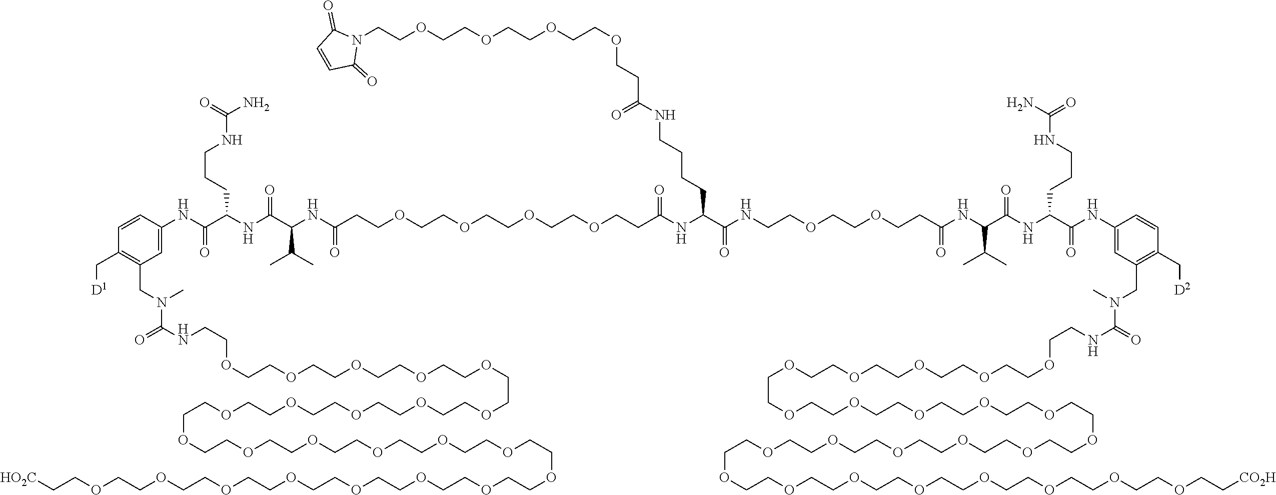

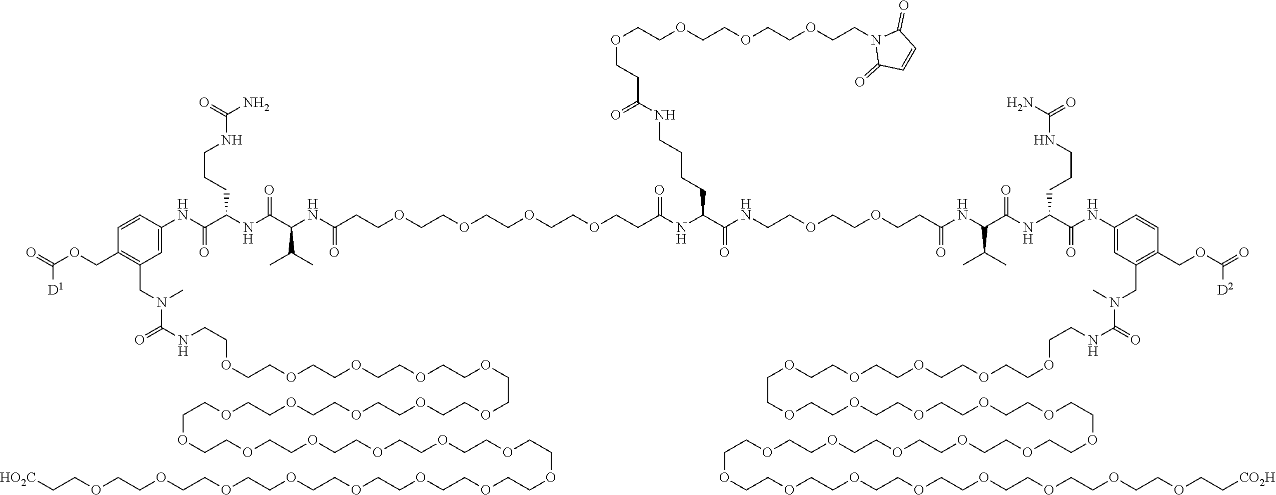

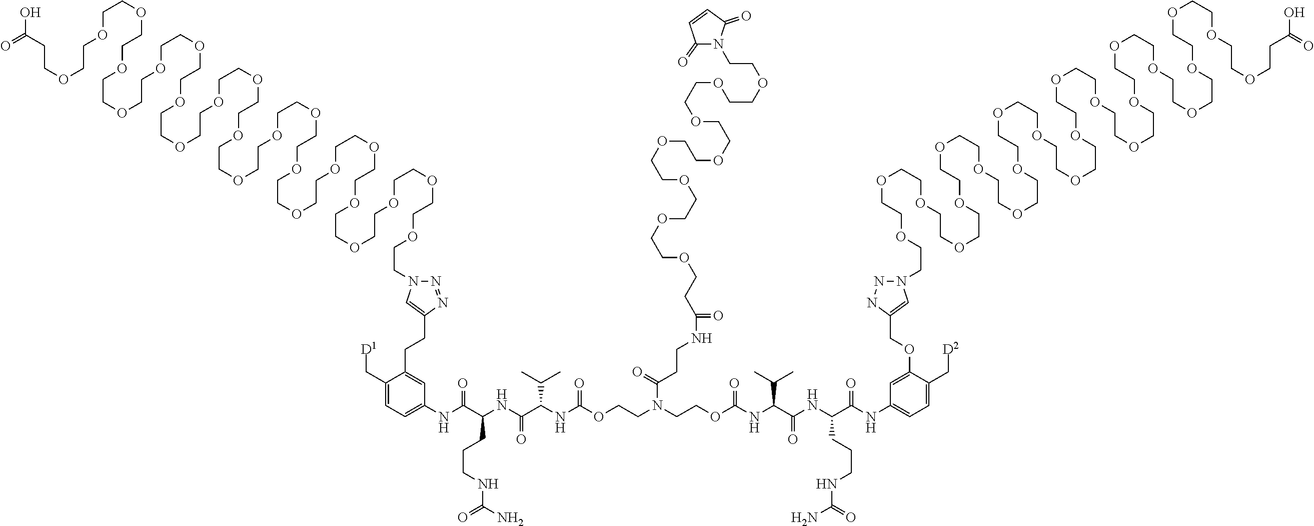

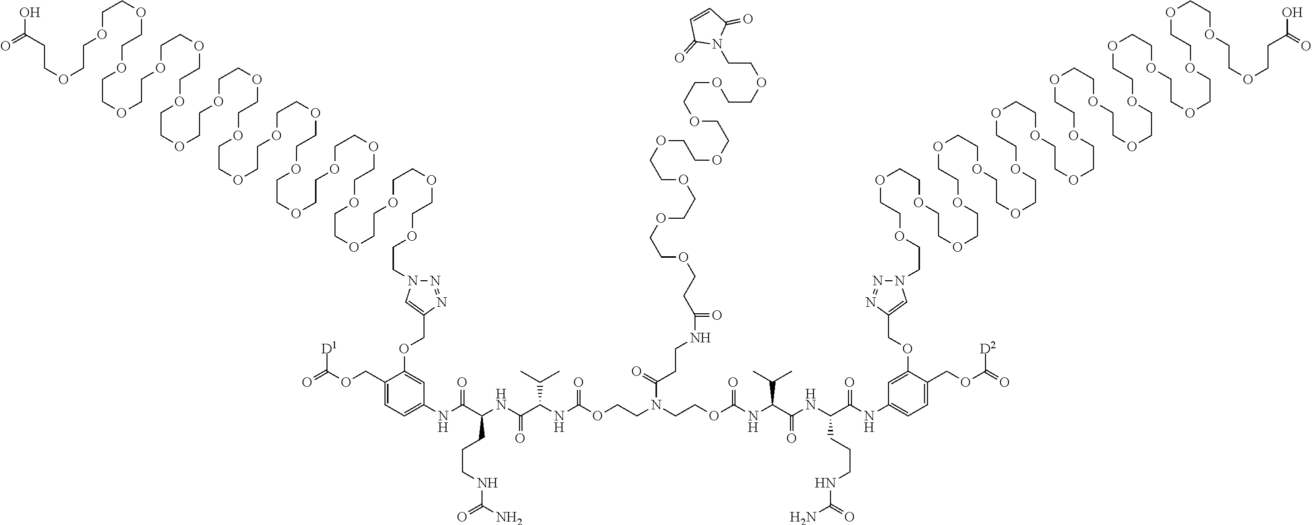

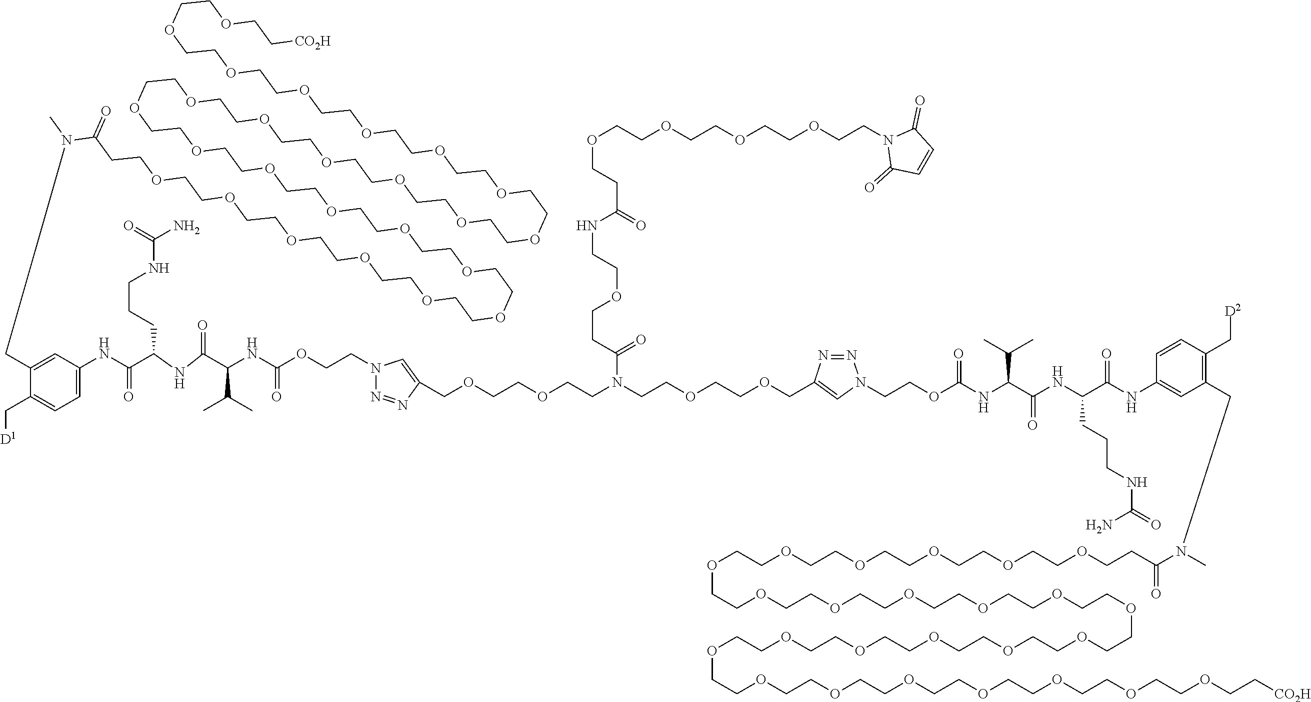

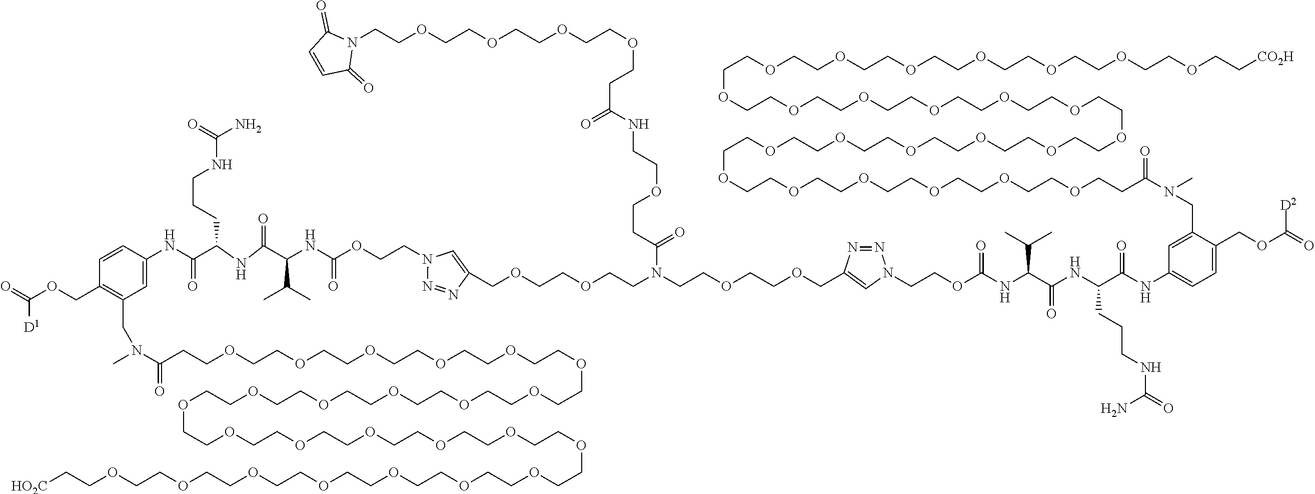

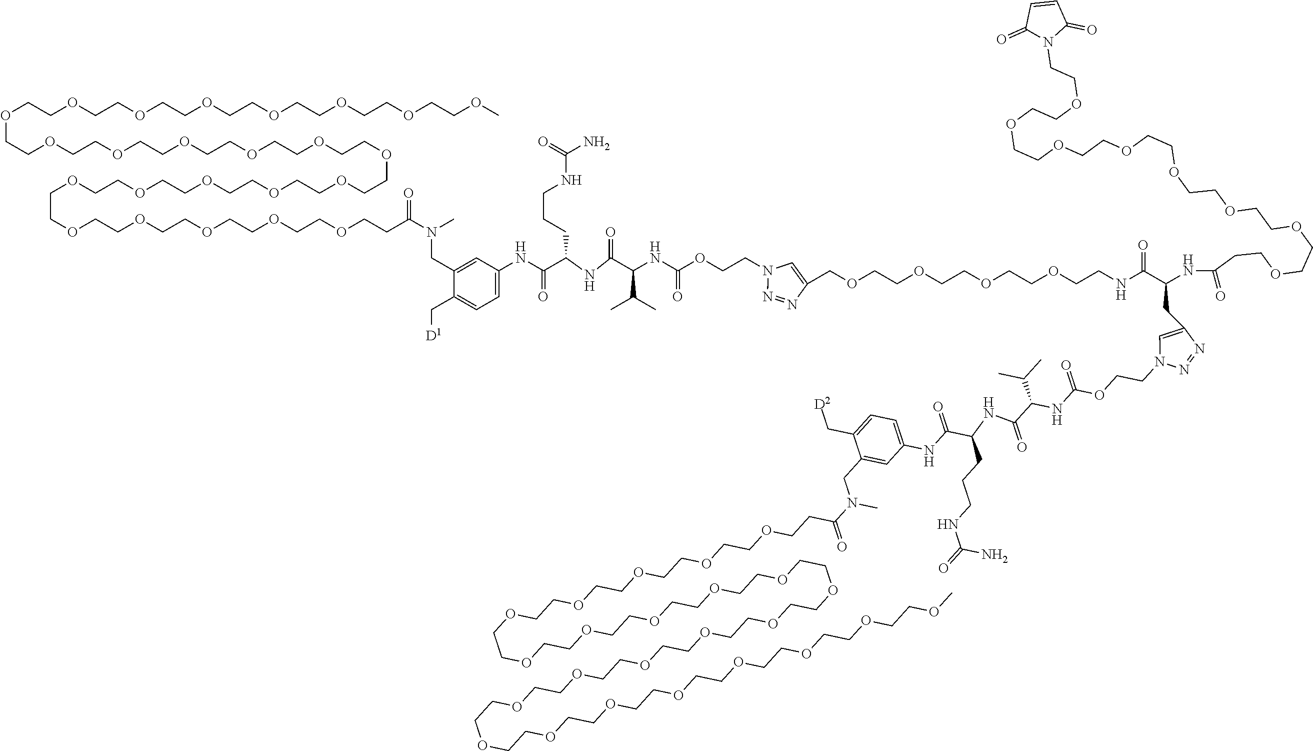

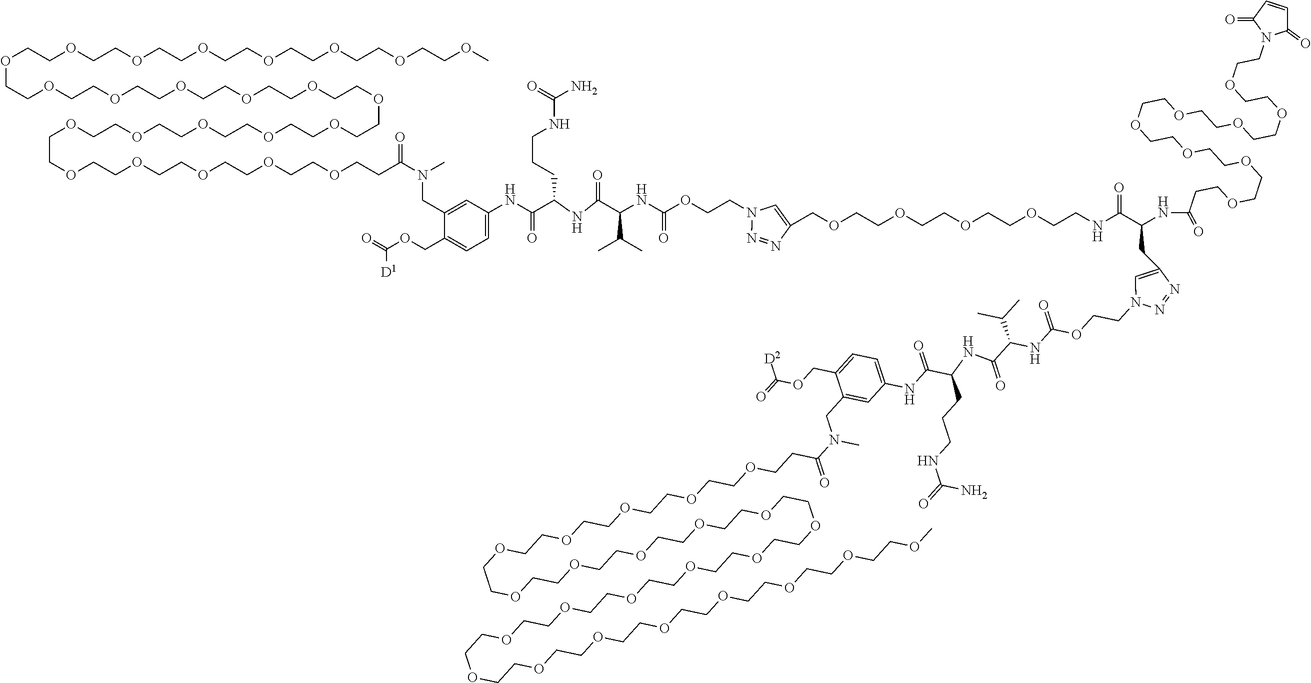

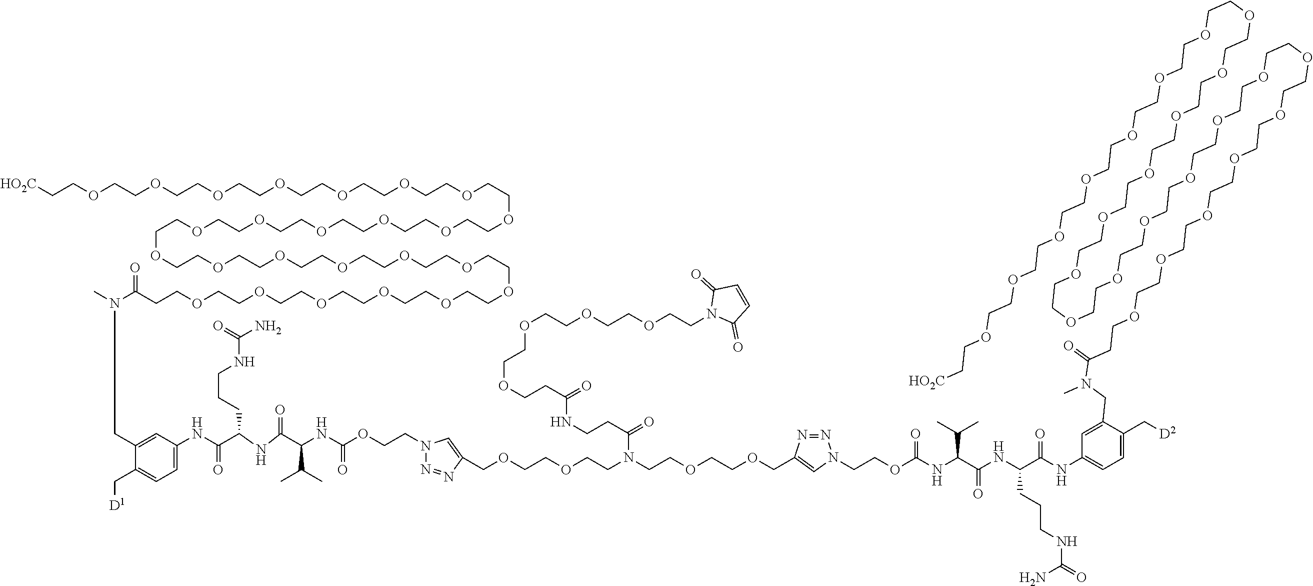

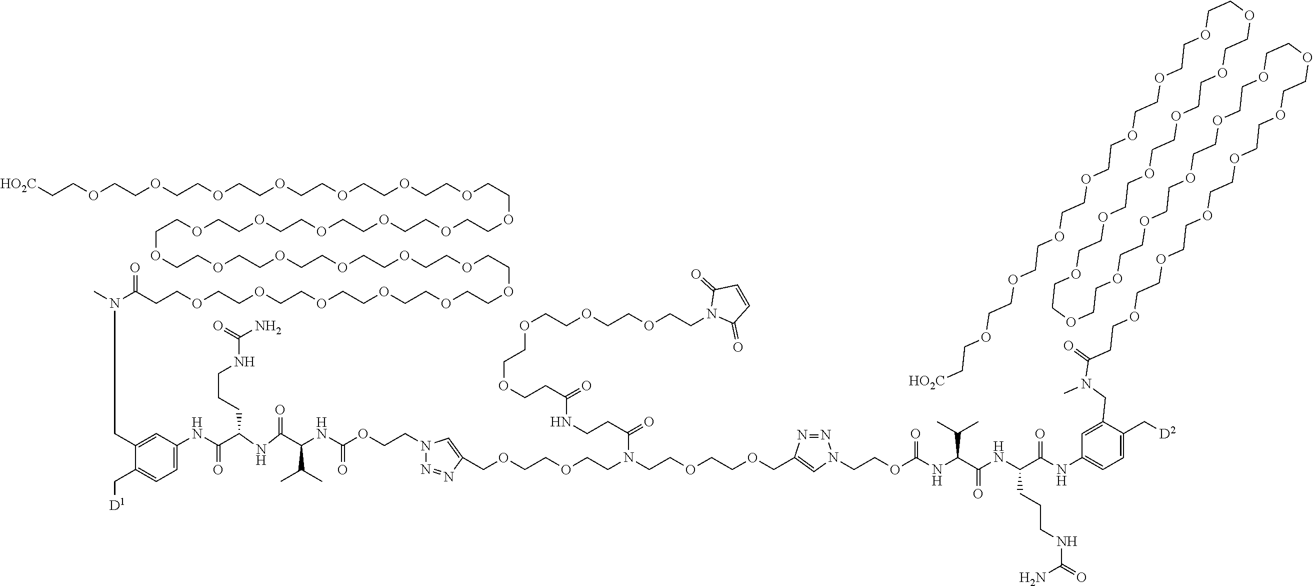

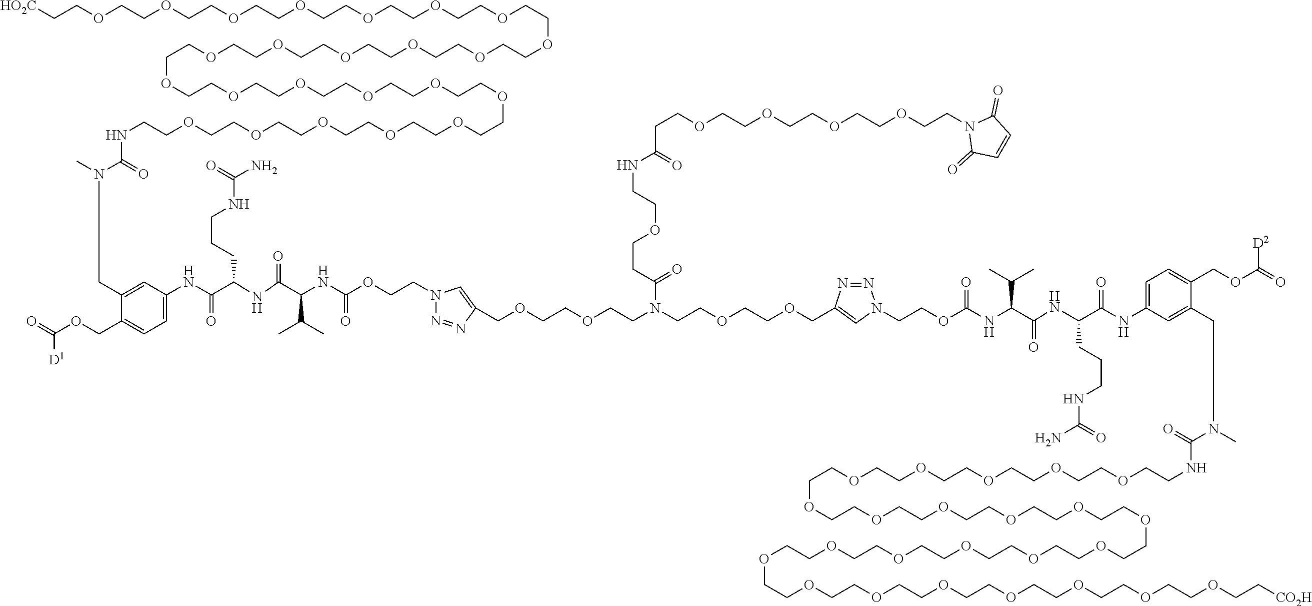

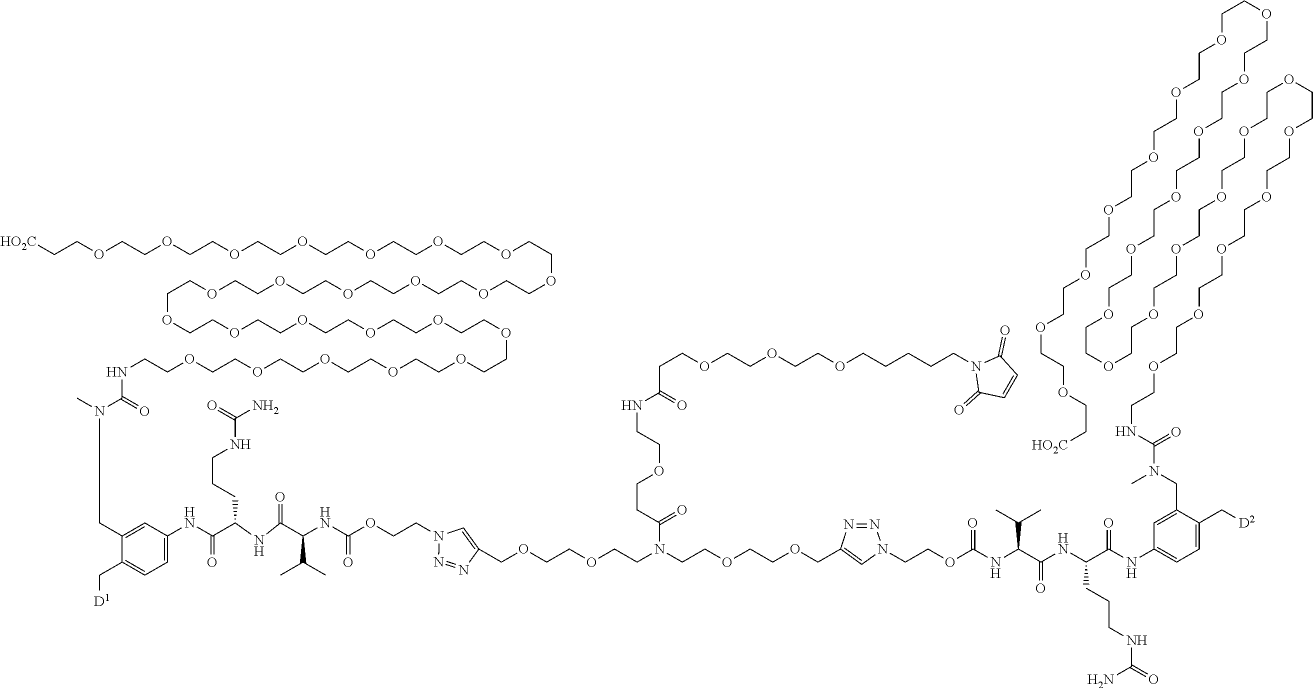

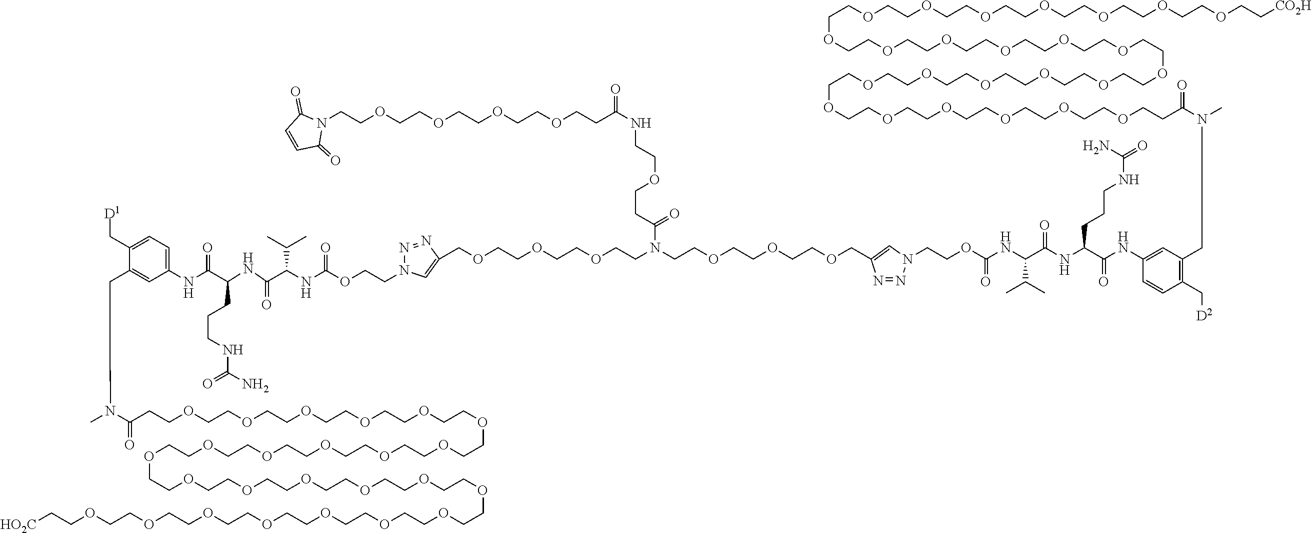

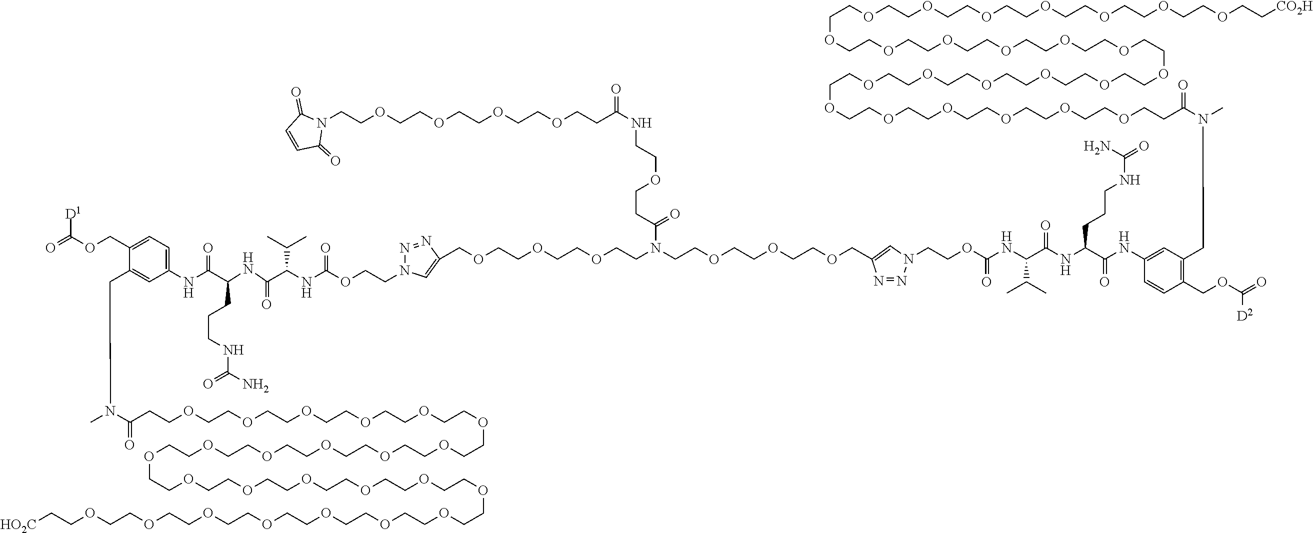

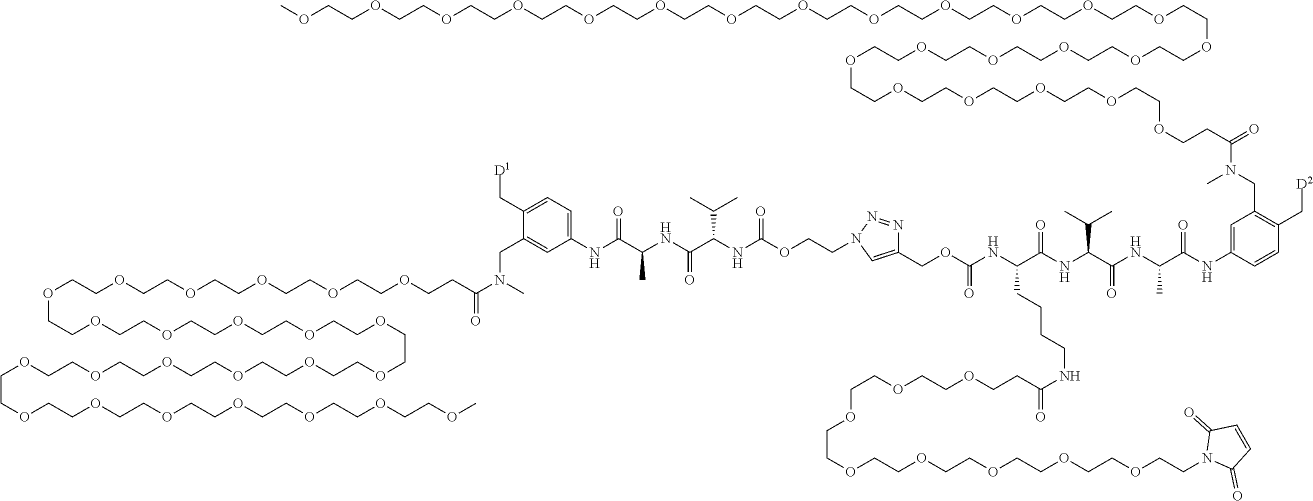

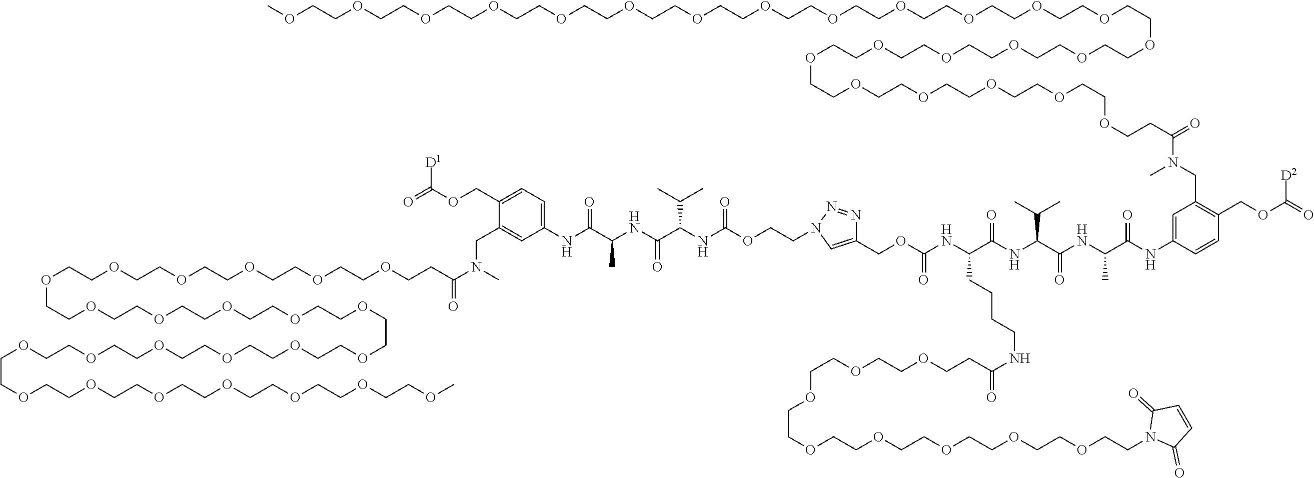

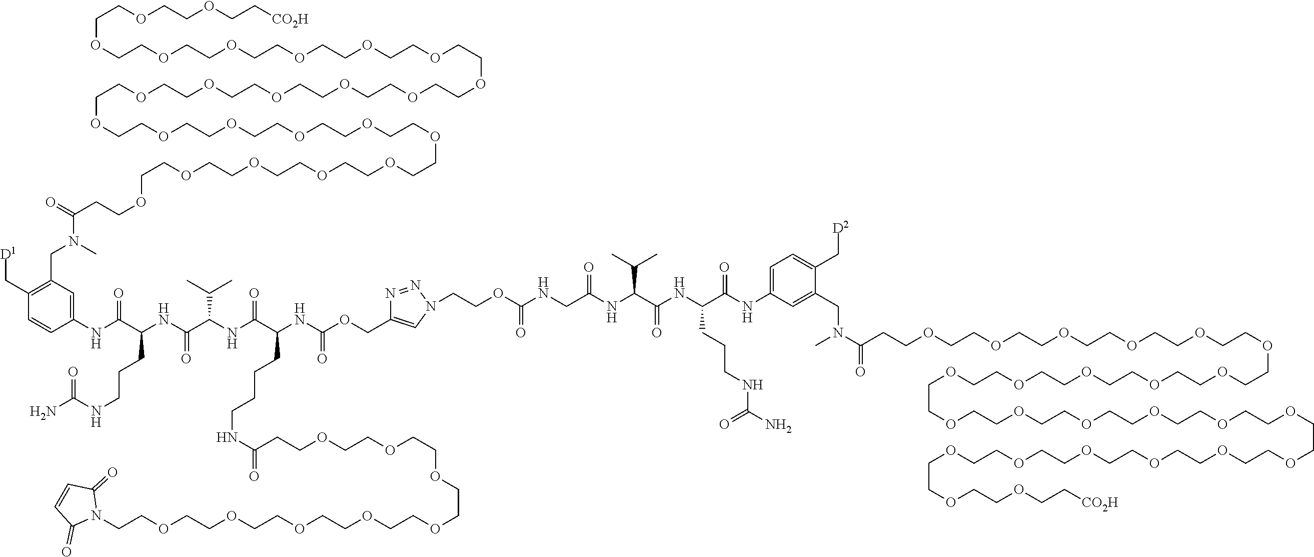

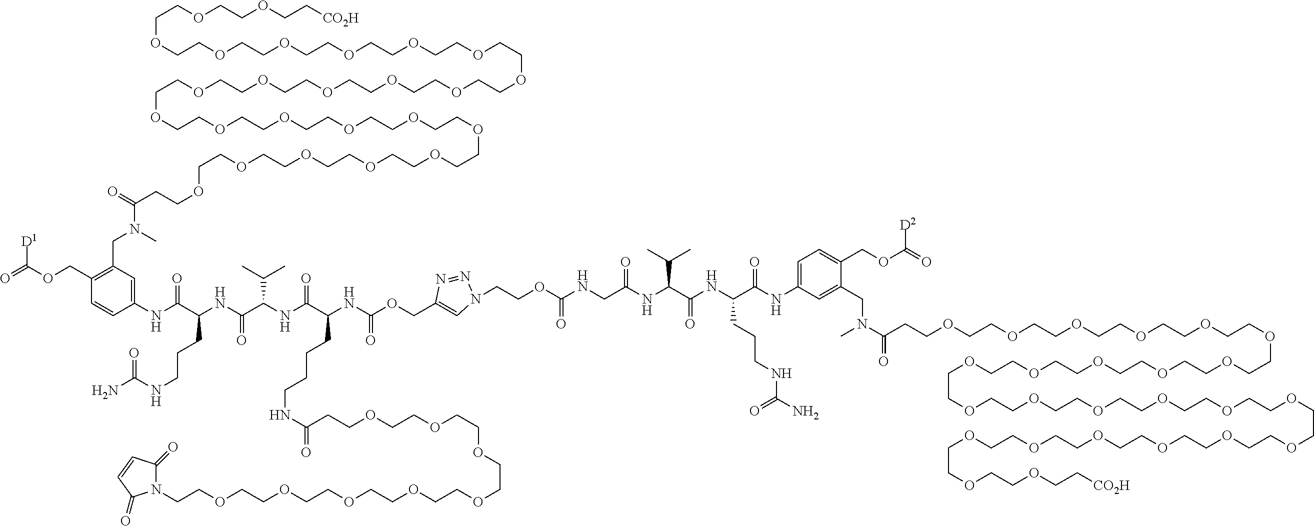

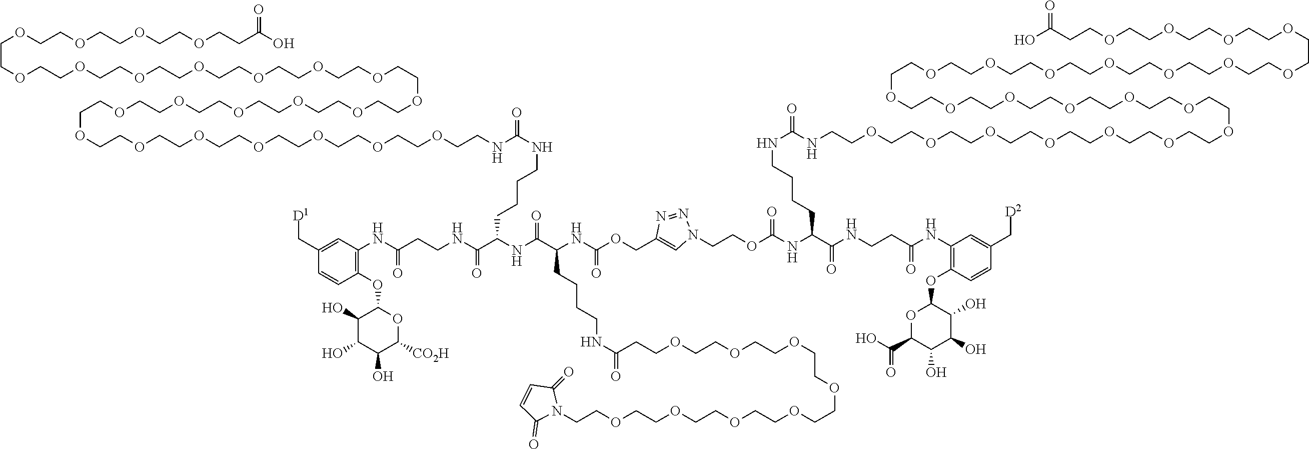

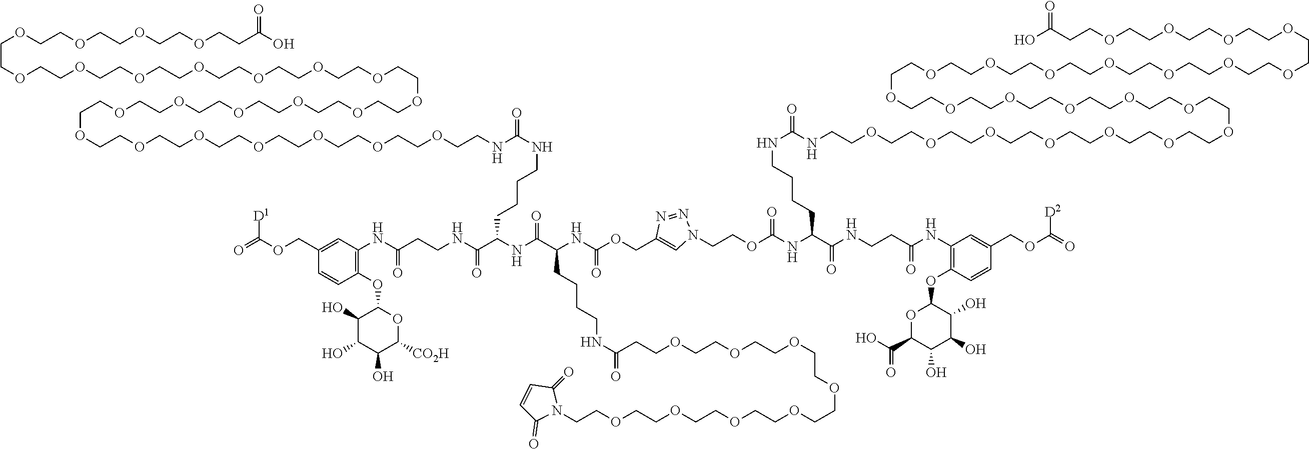

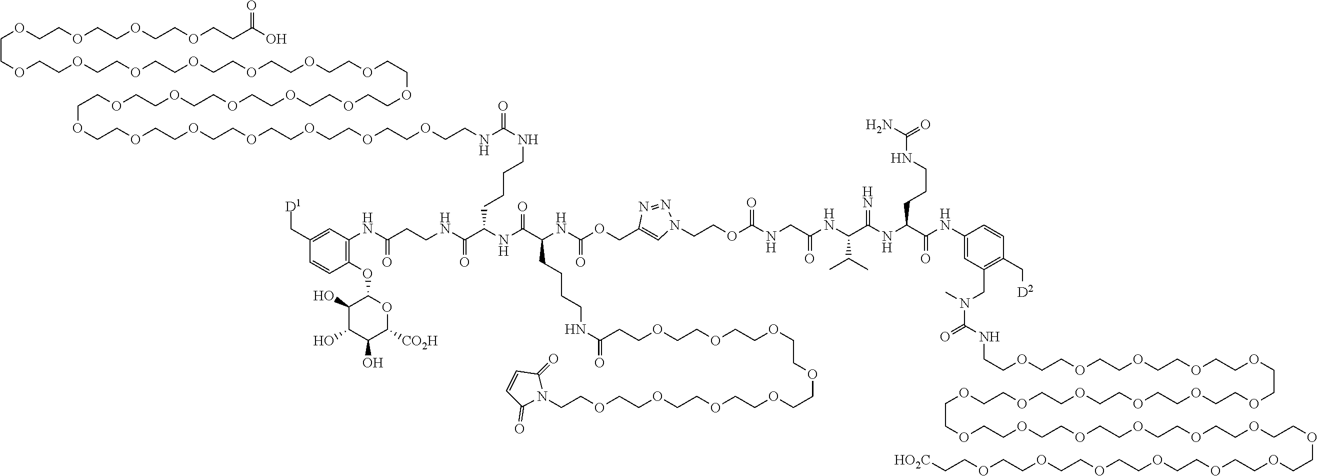

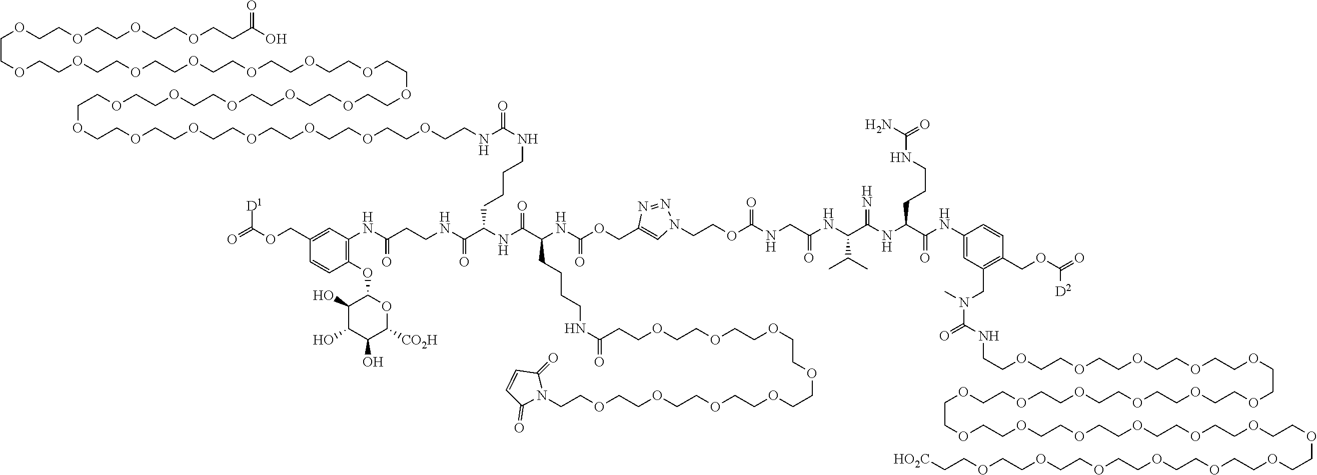

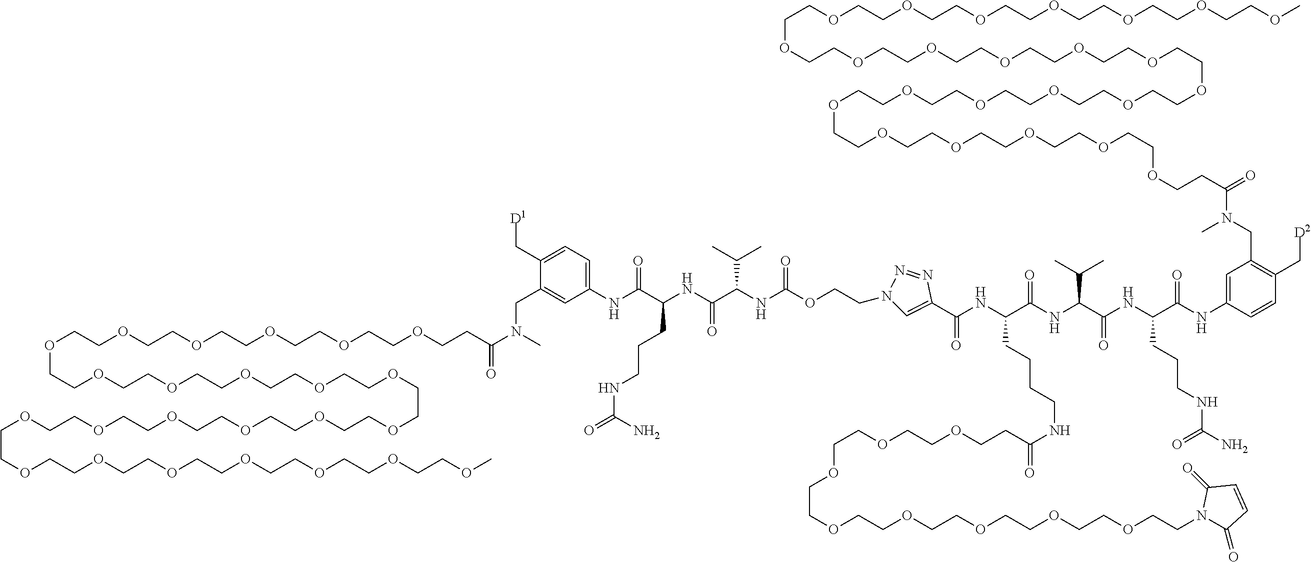

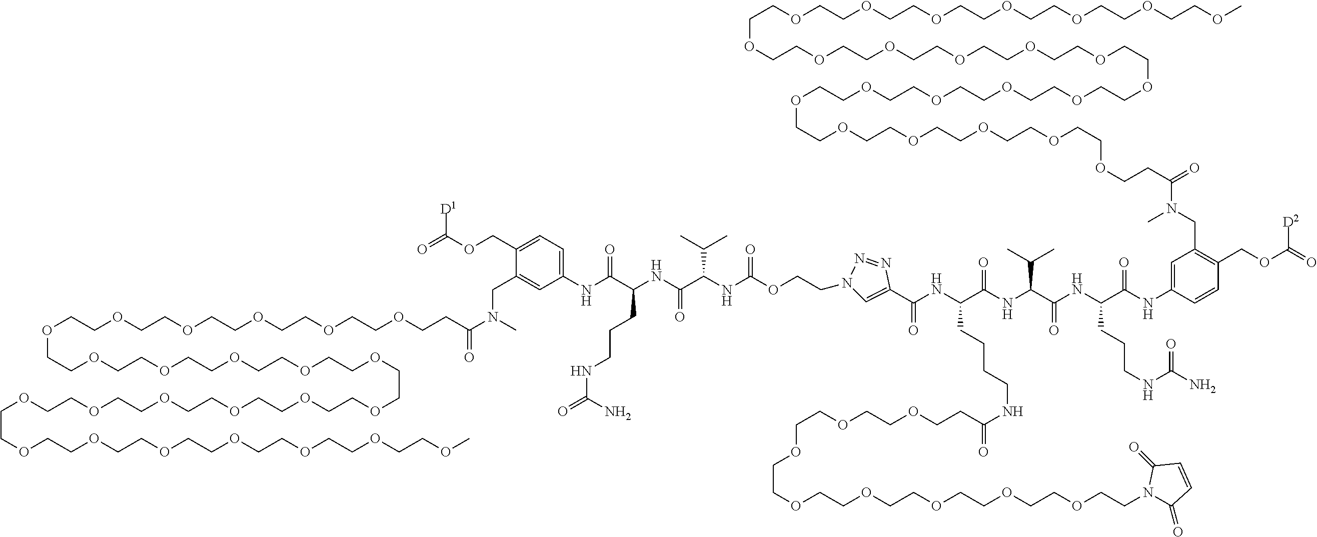

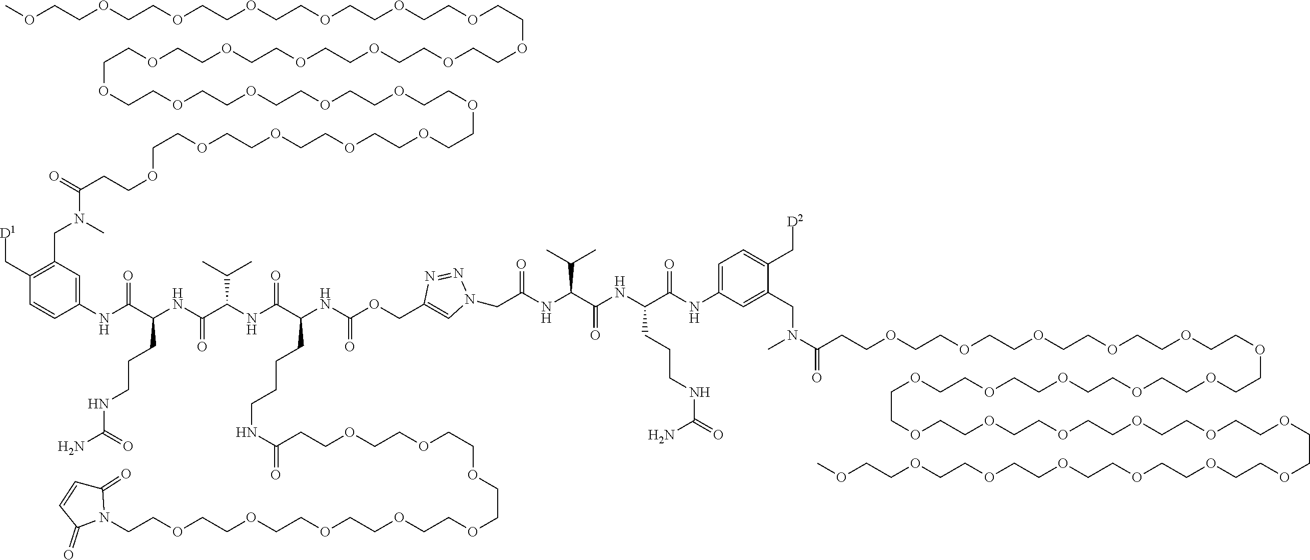

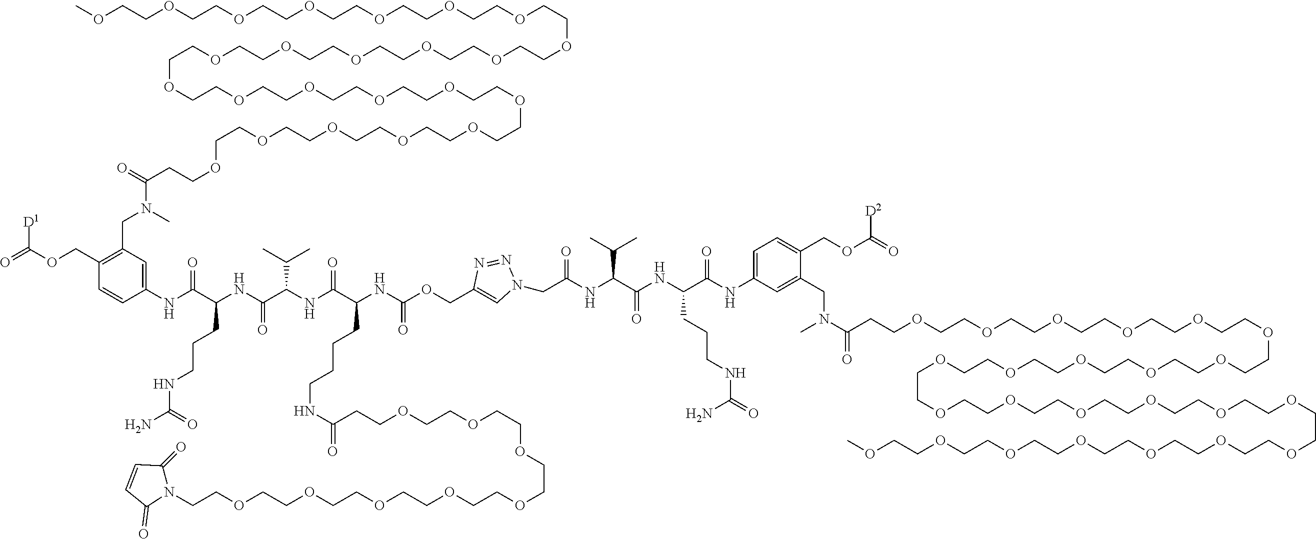

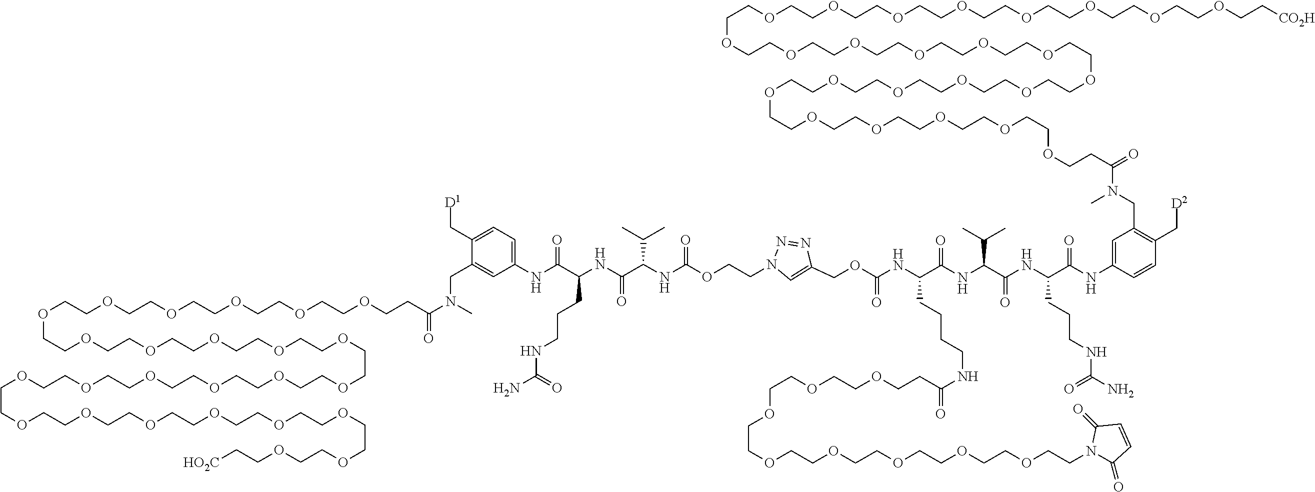

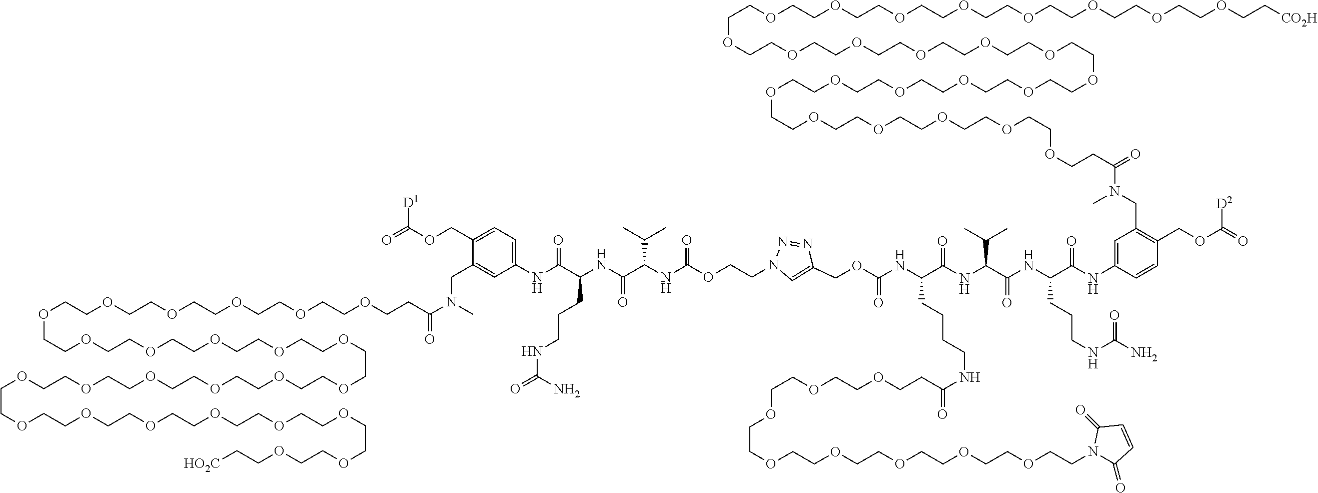

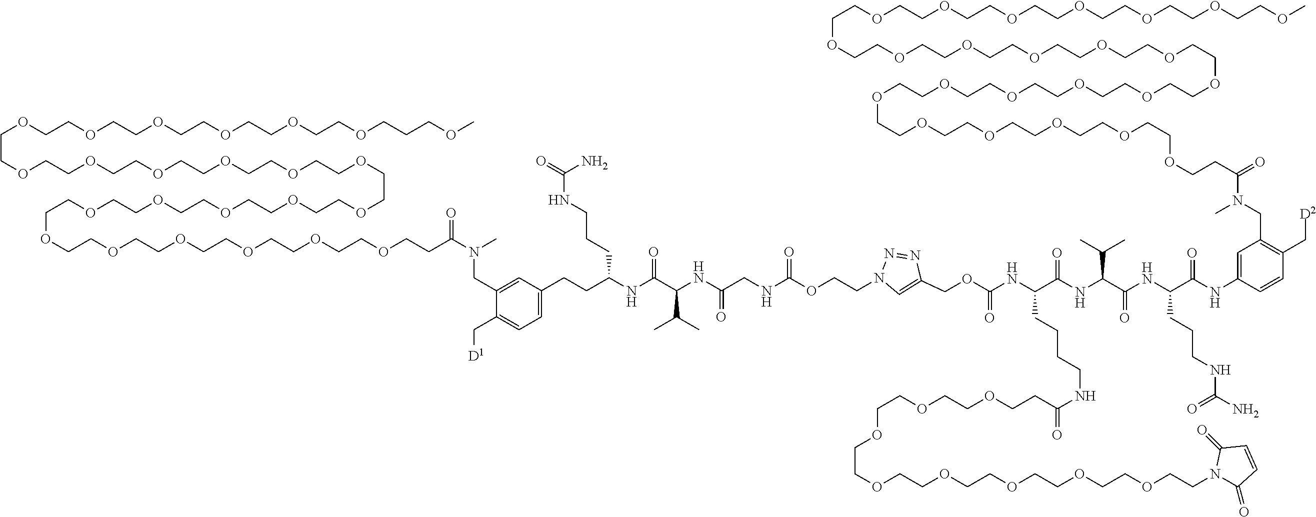

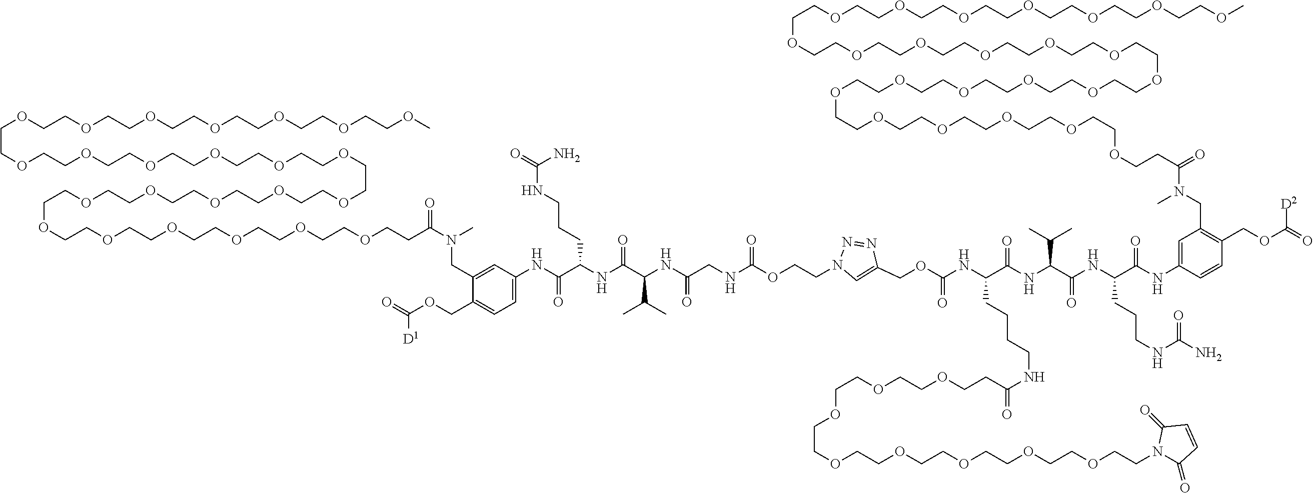

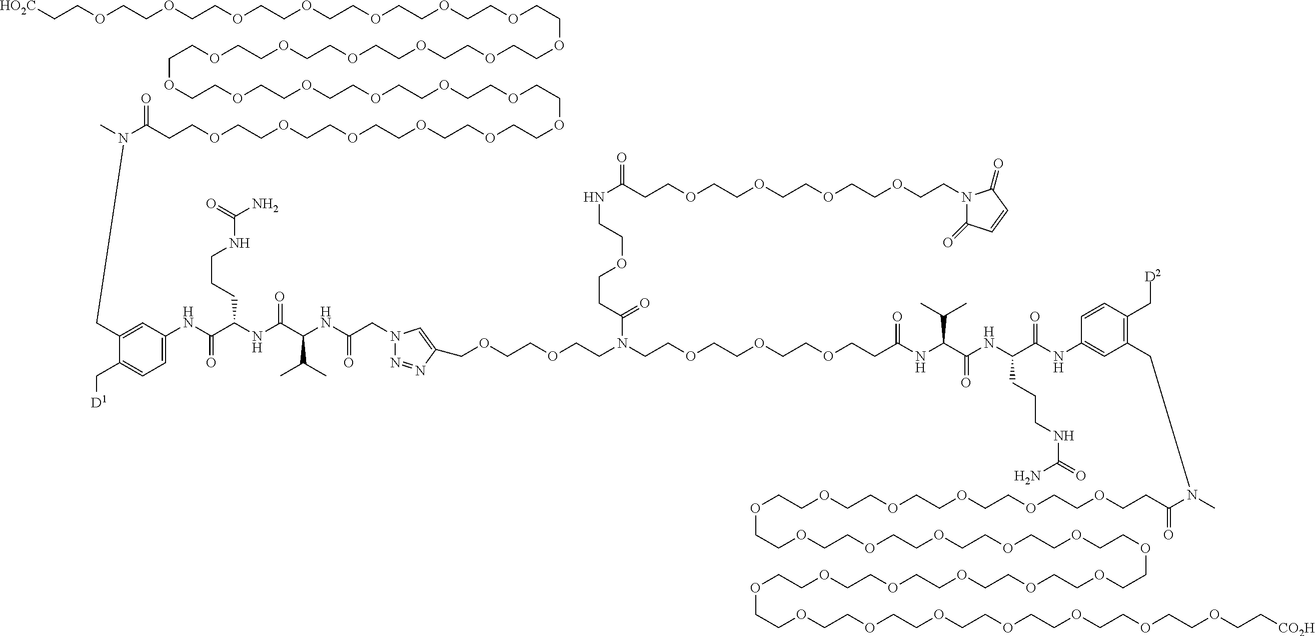

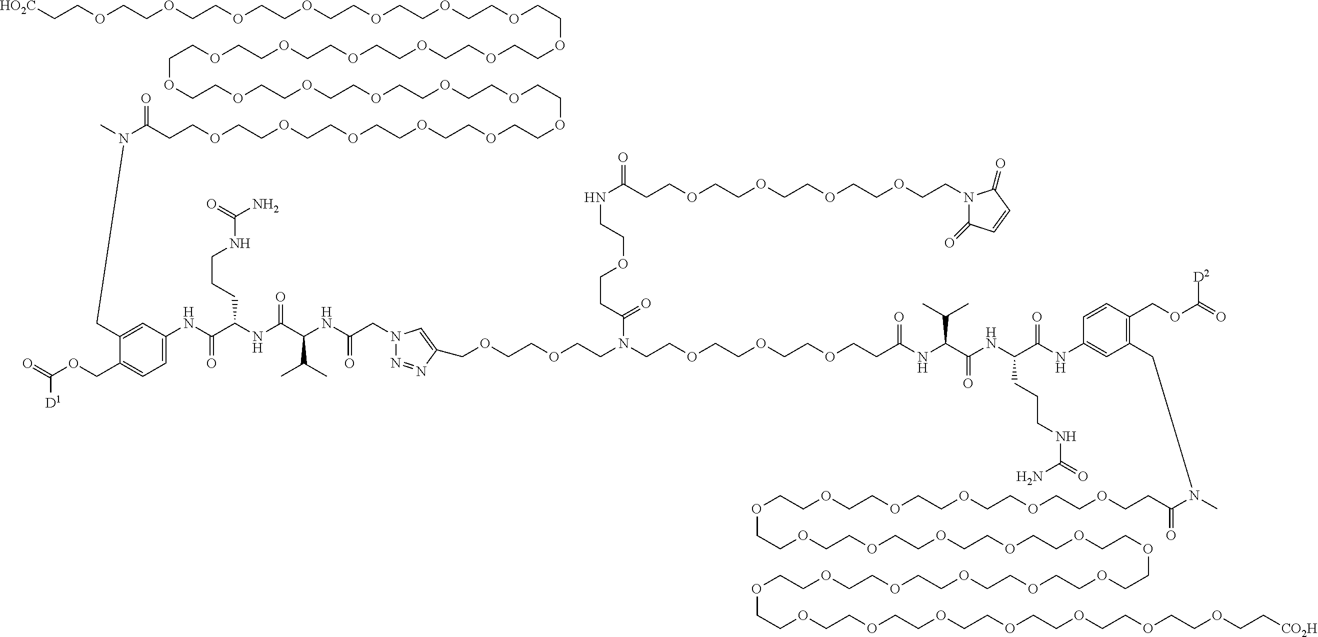

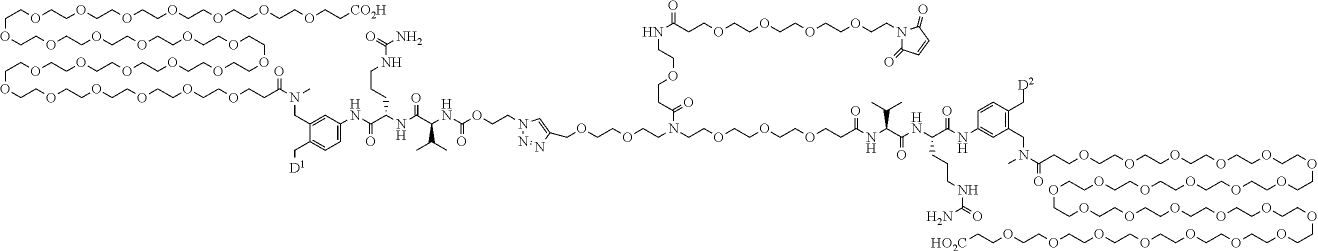

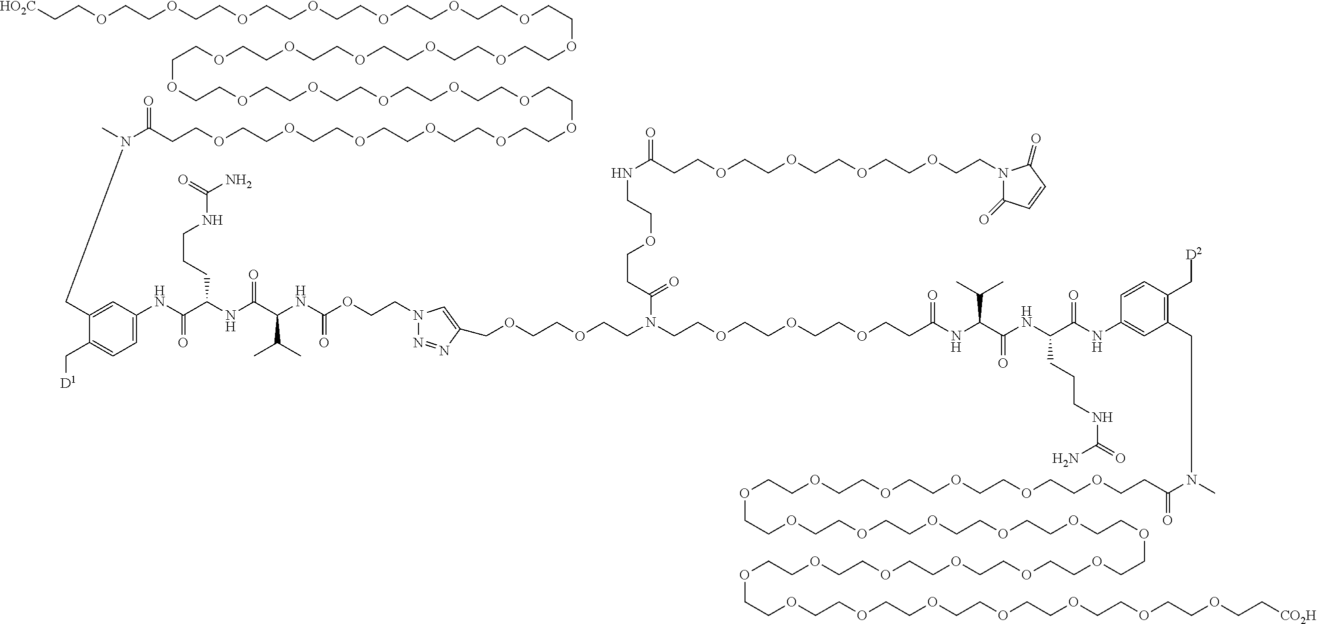

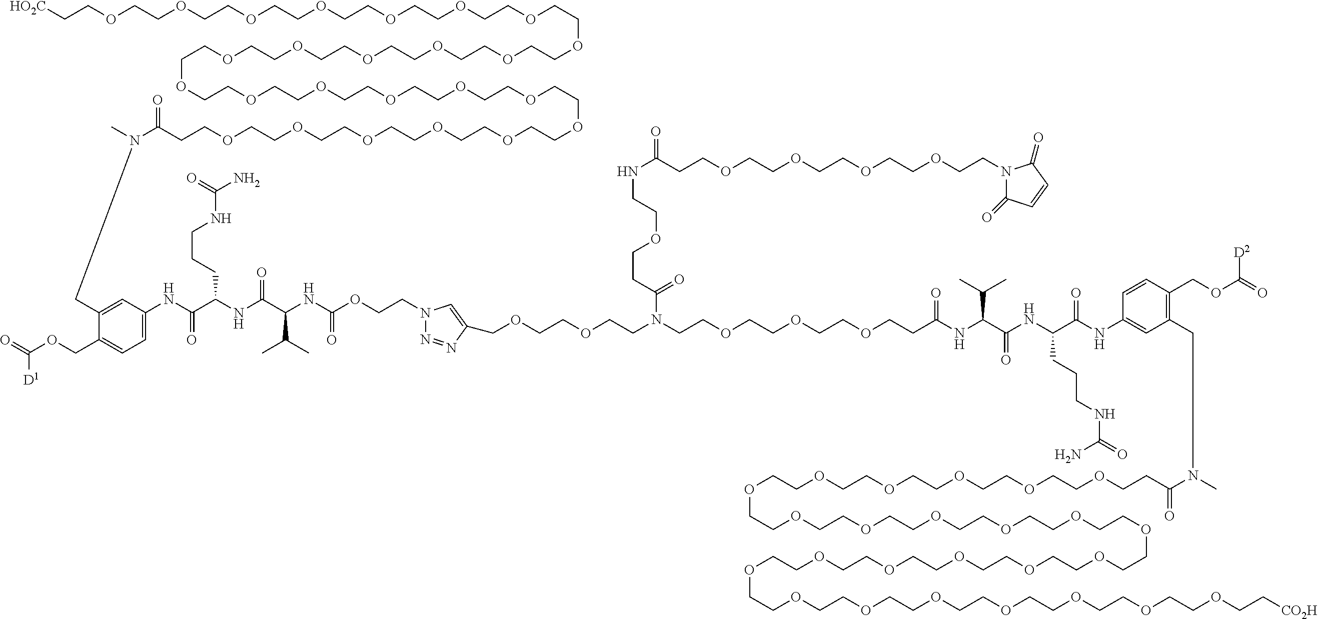

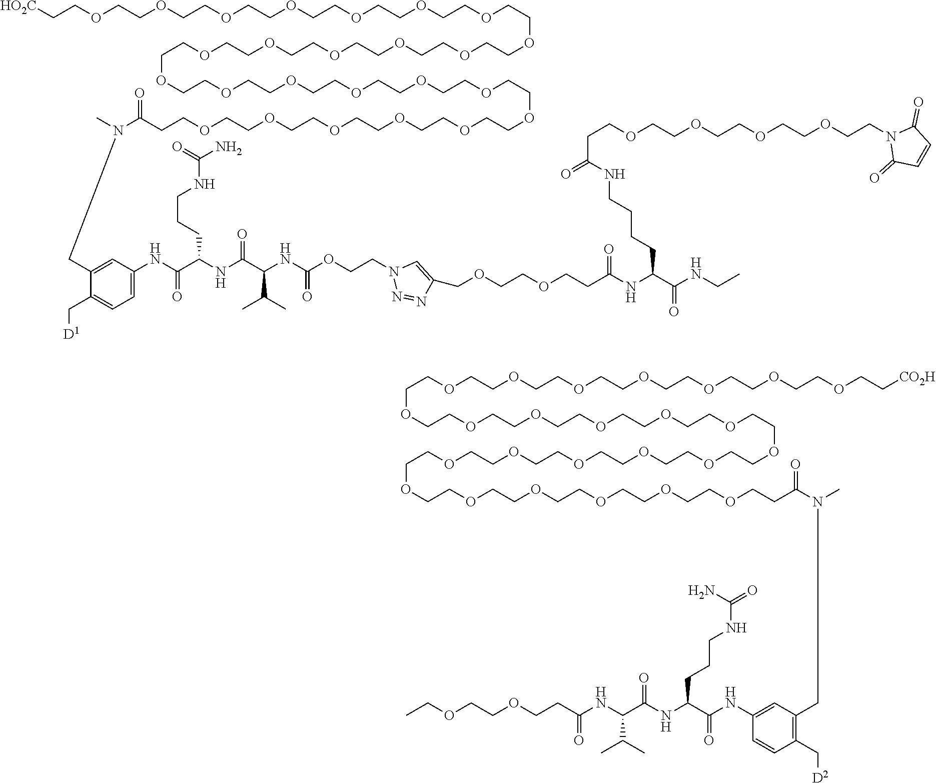

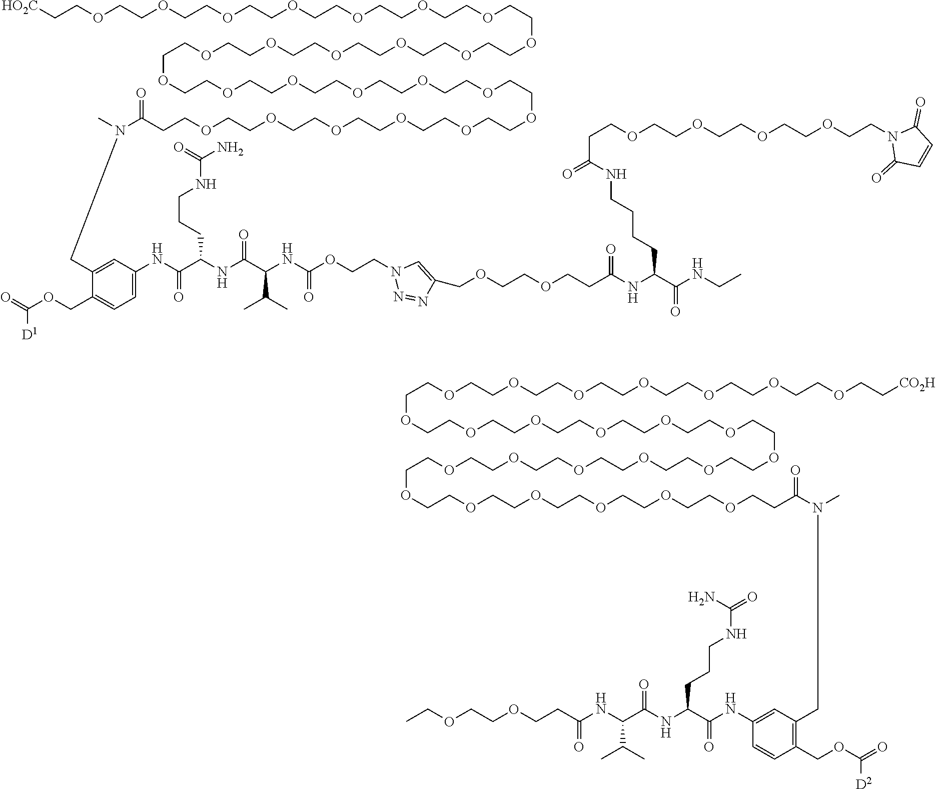

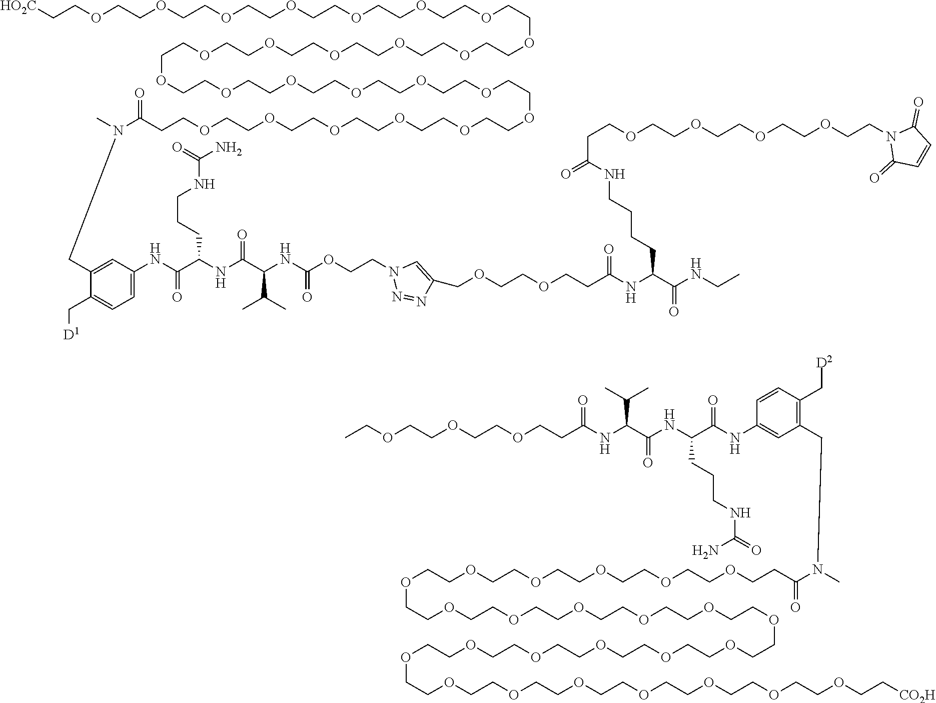

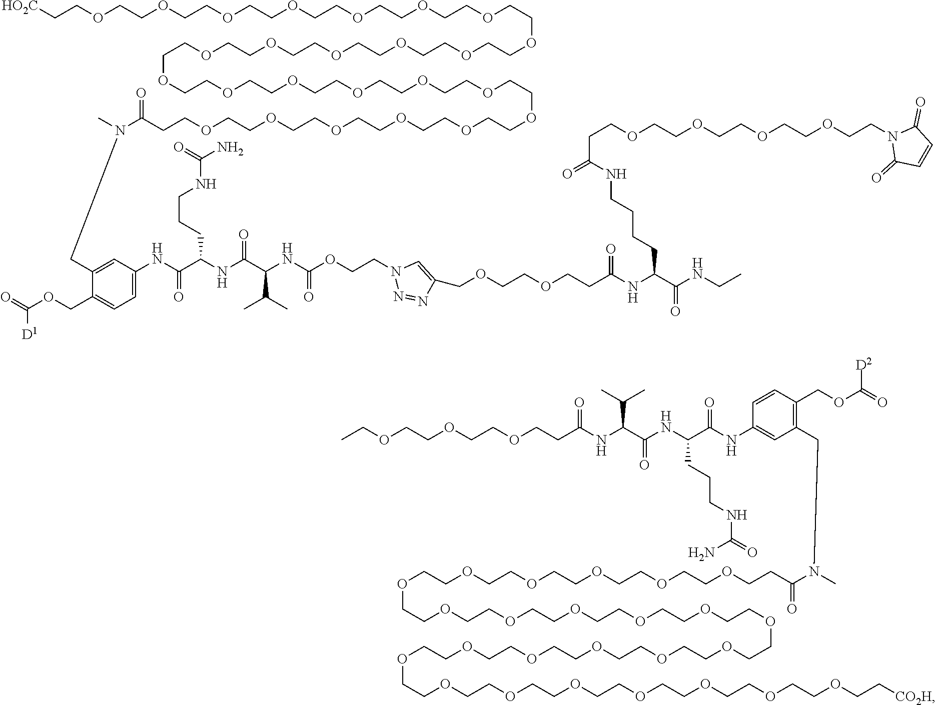

or a pharmaceutically acceptable salt thereof.
Embodiment 44a. The compound or pharmaceutically acceptable salt thereof according to any one of the preceding Embodiments, selected from the group consisting of:

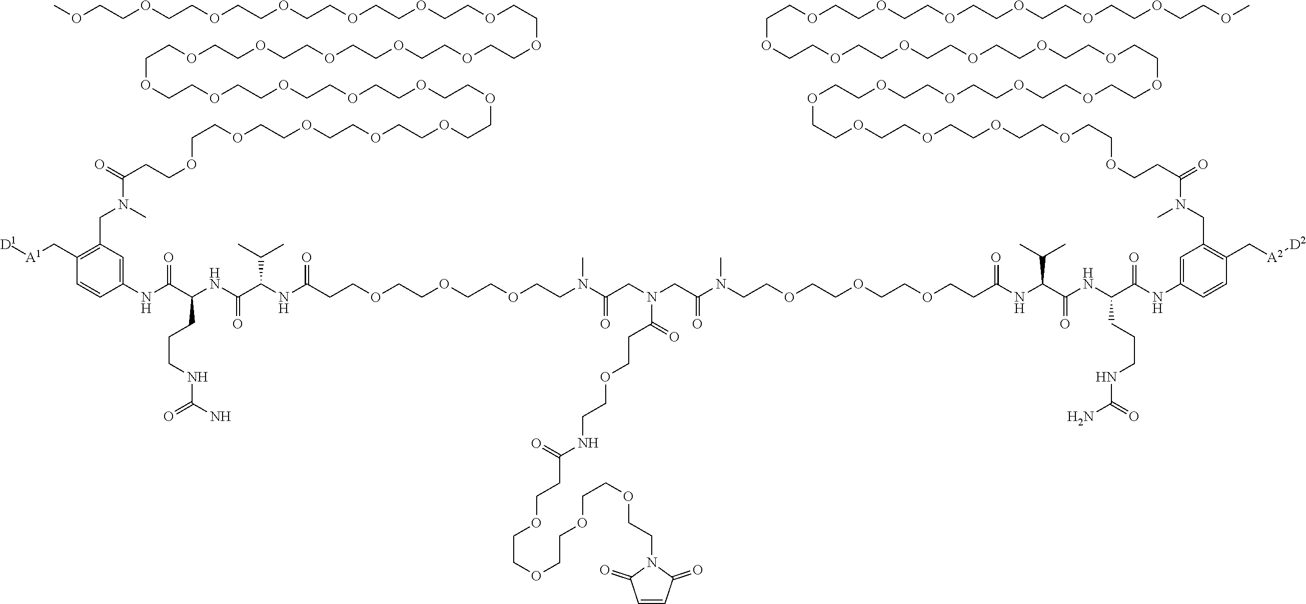

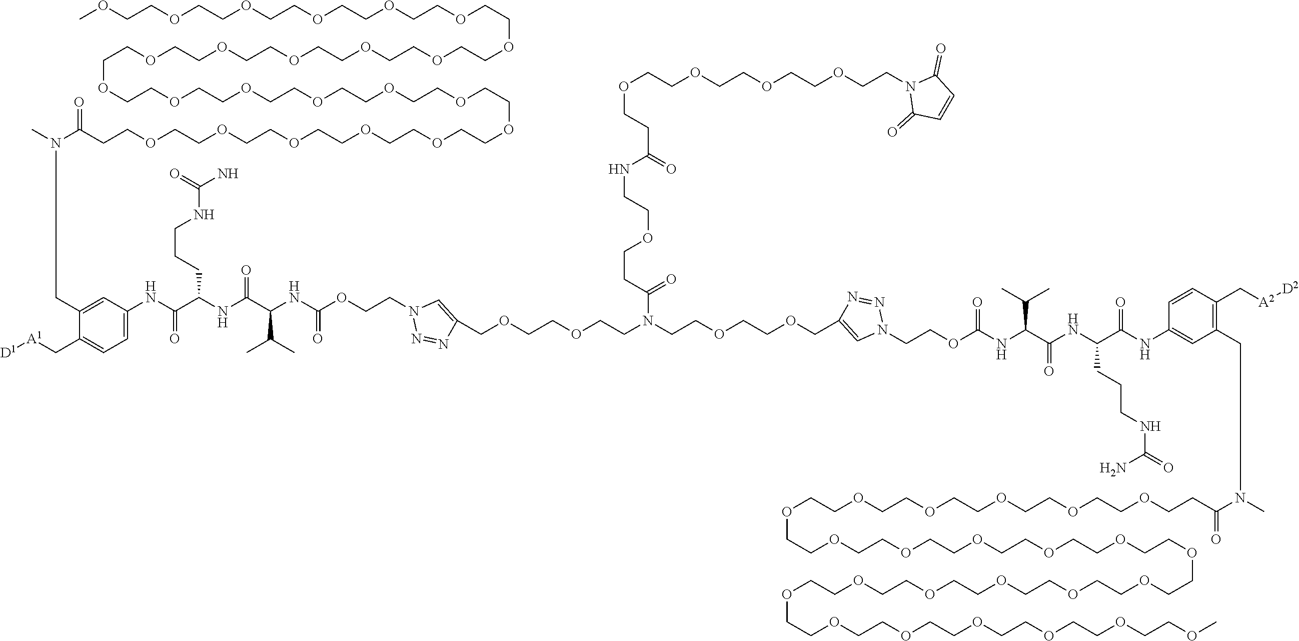

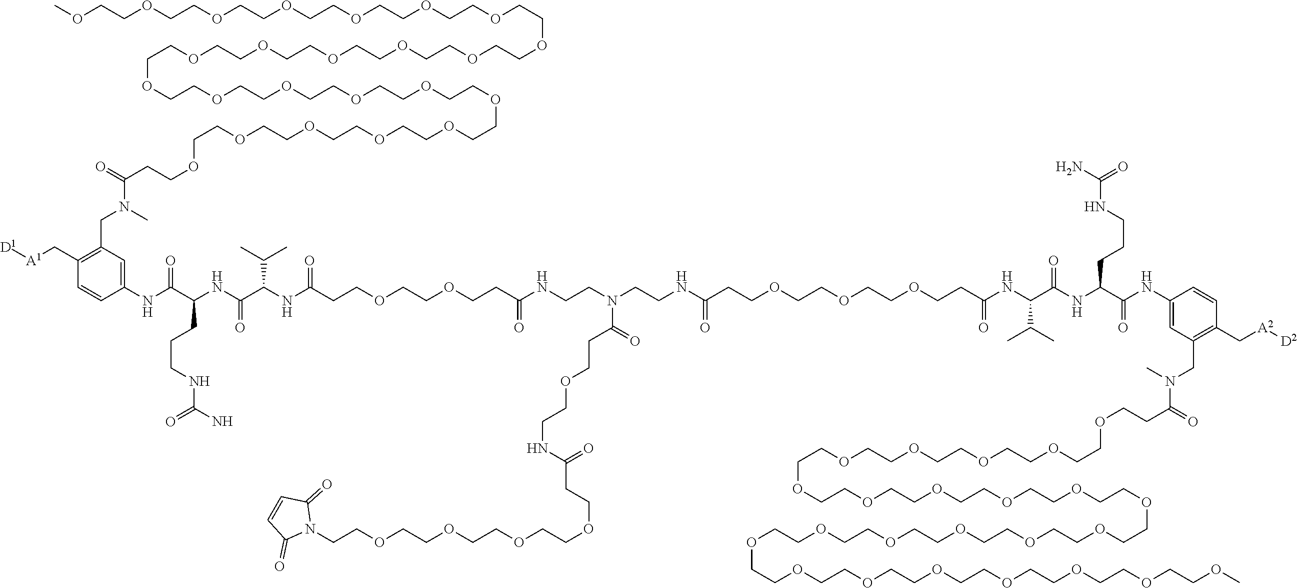

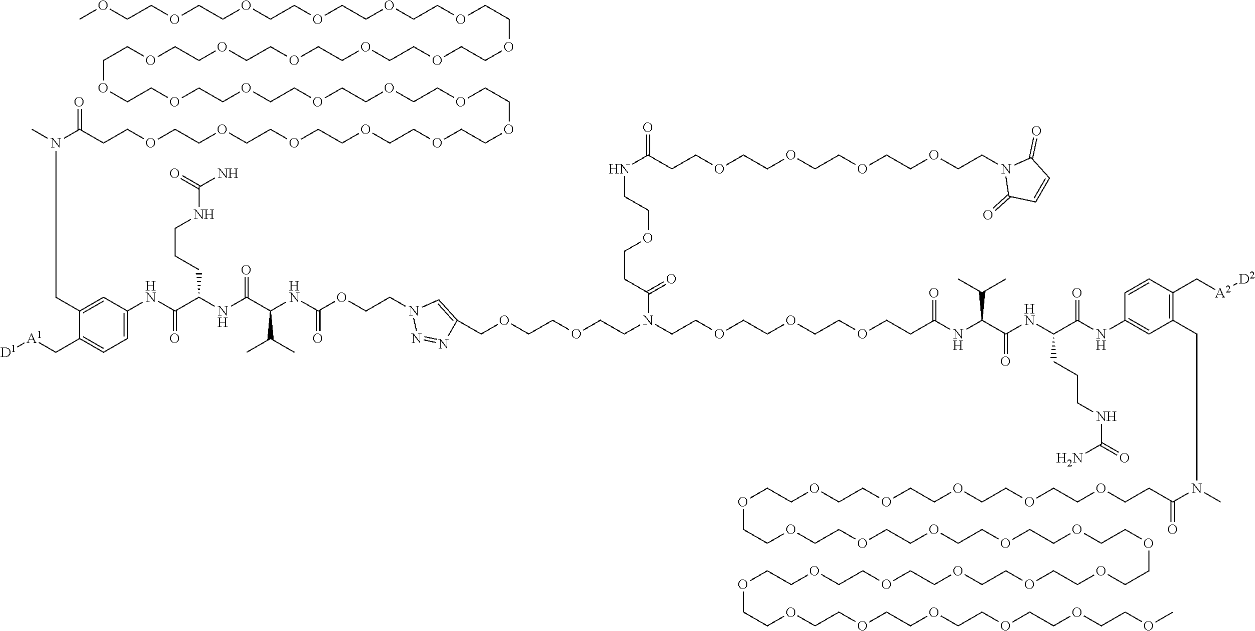

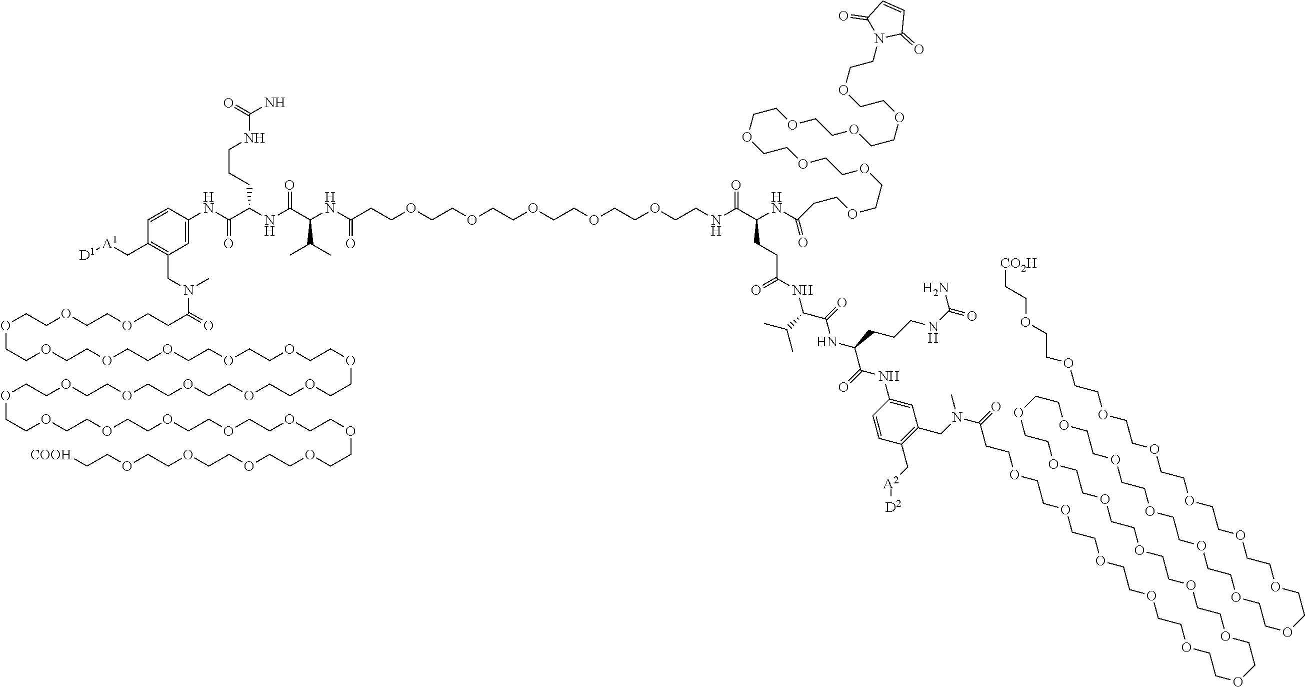

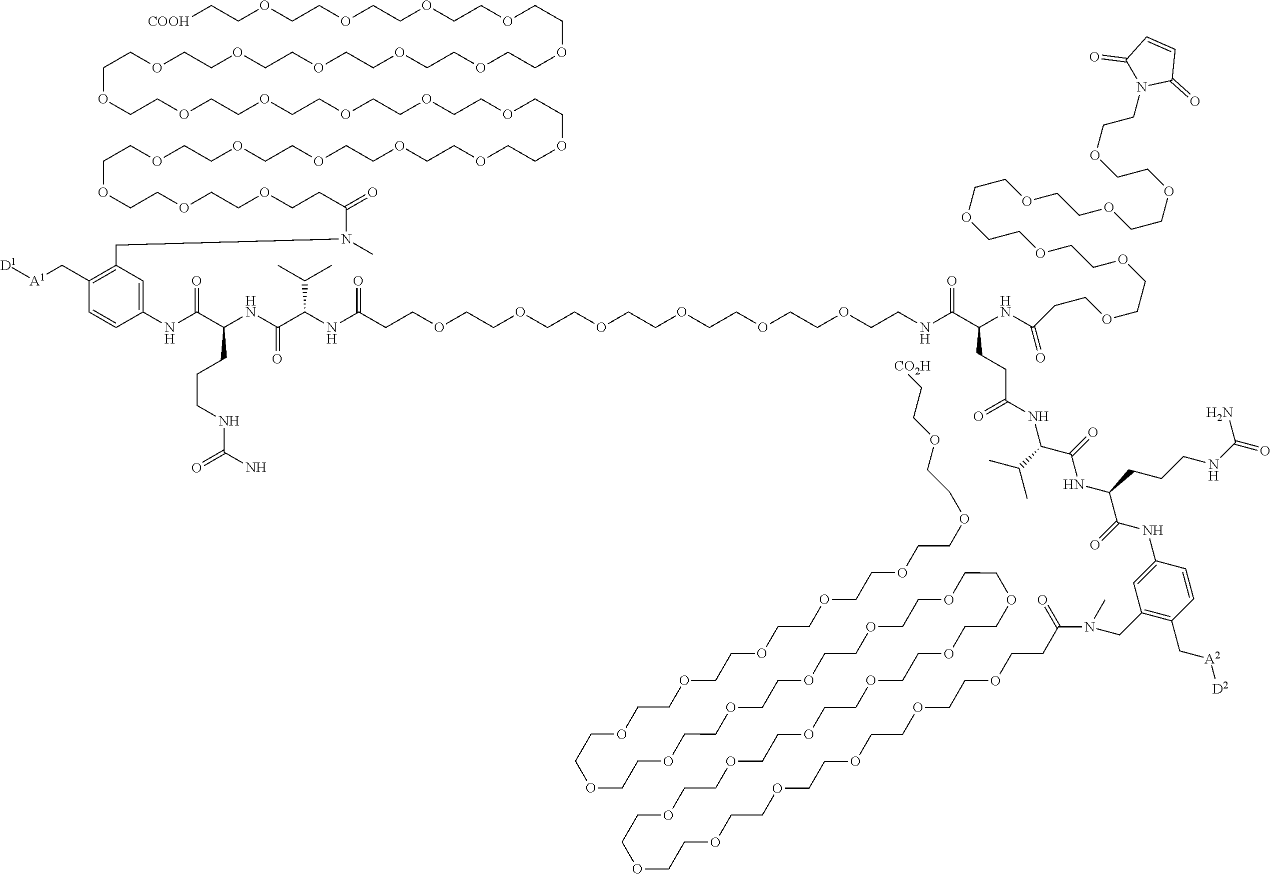

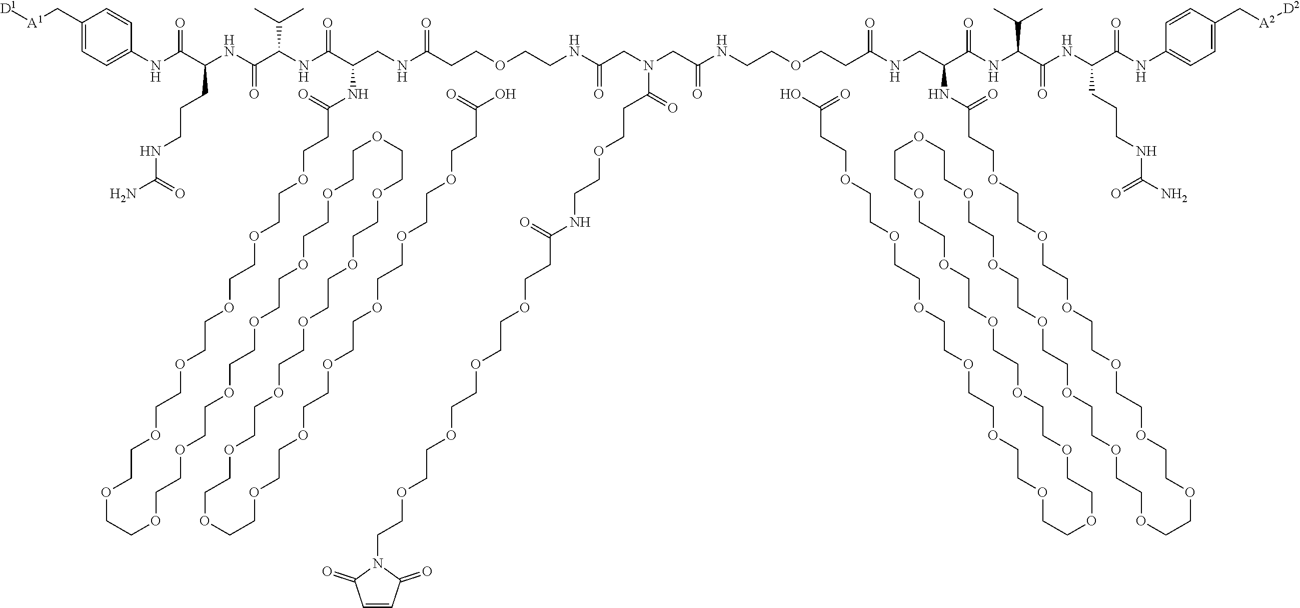

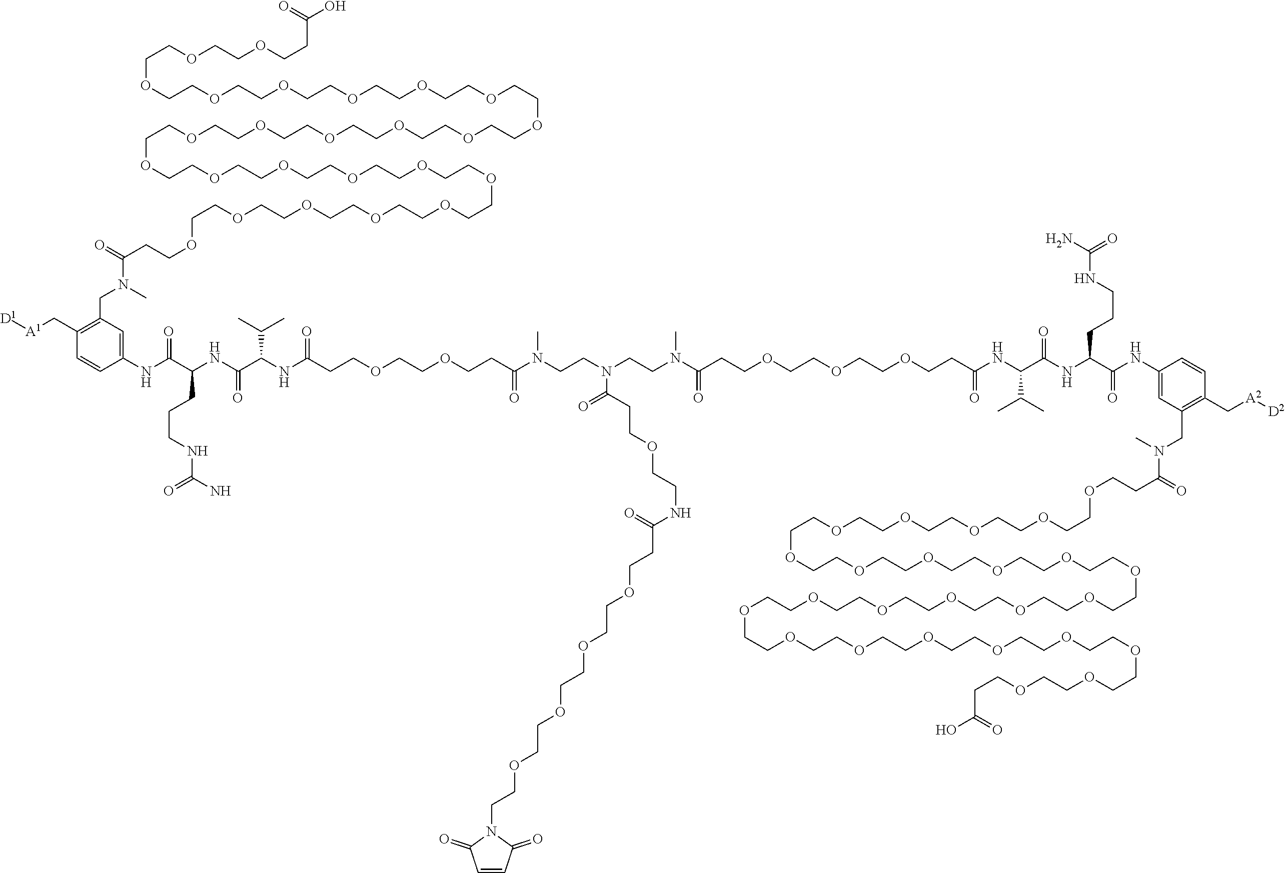

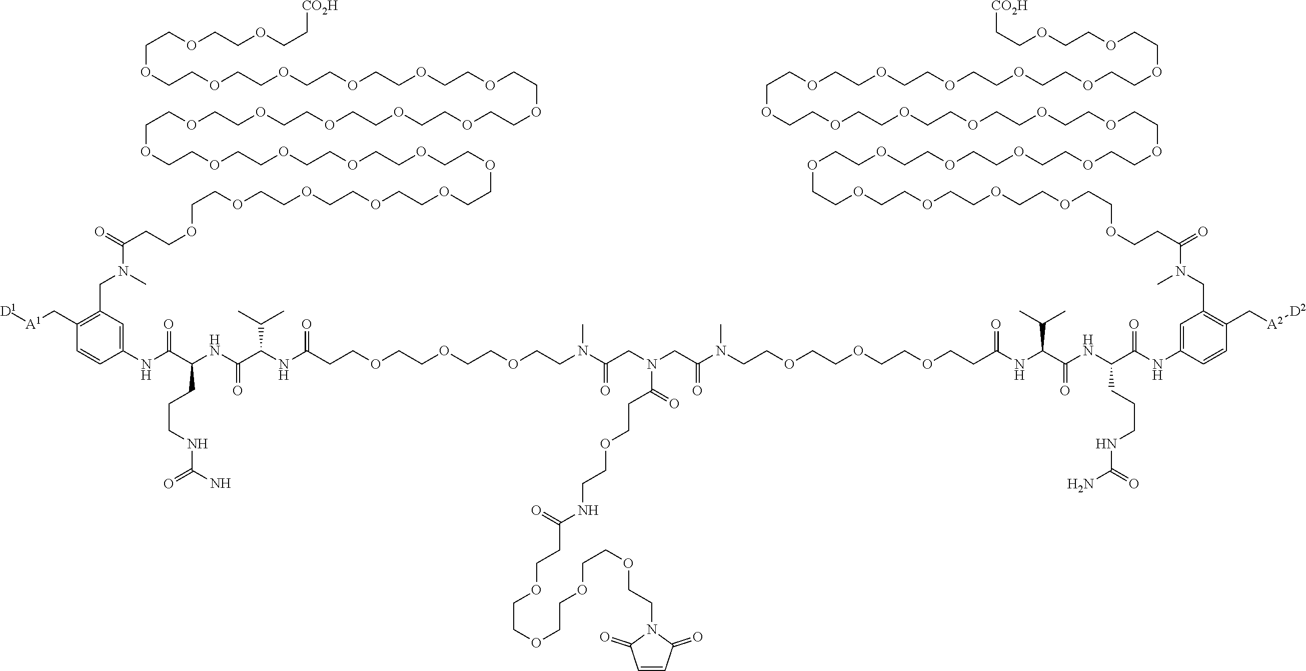

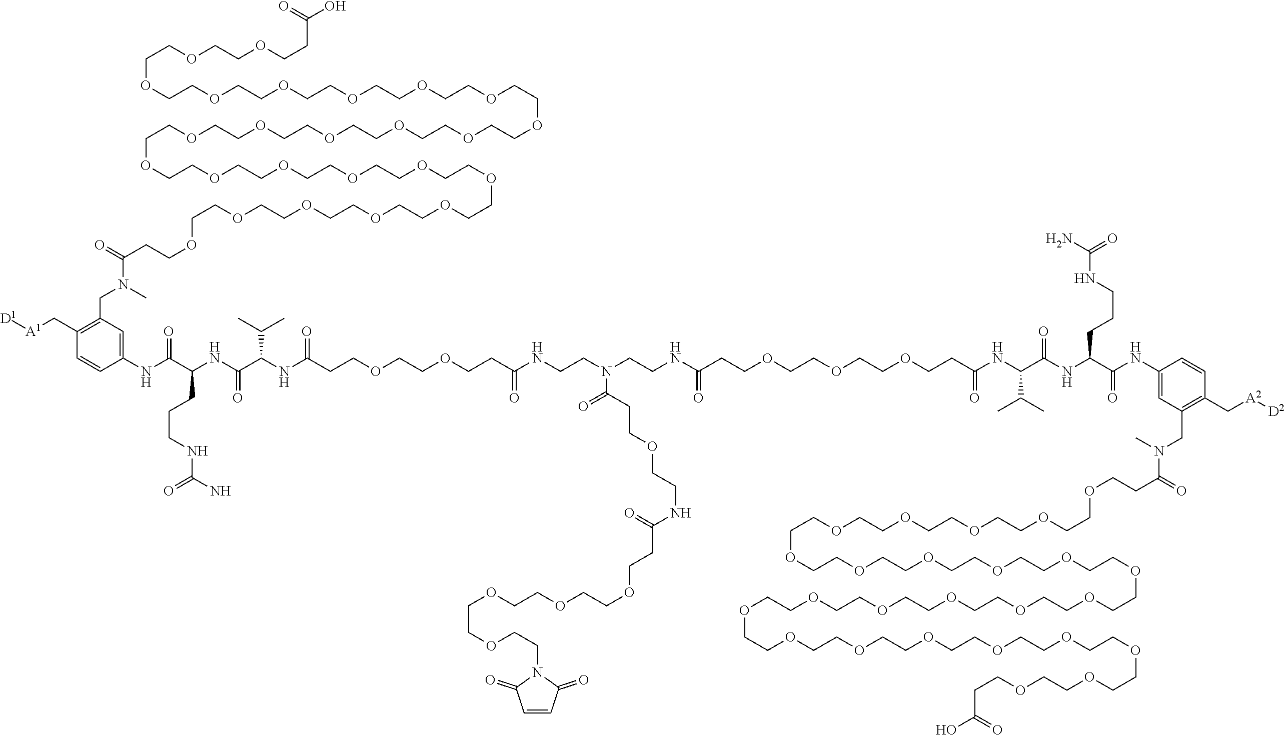

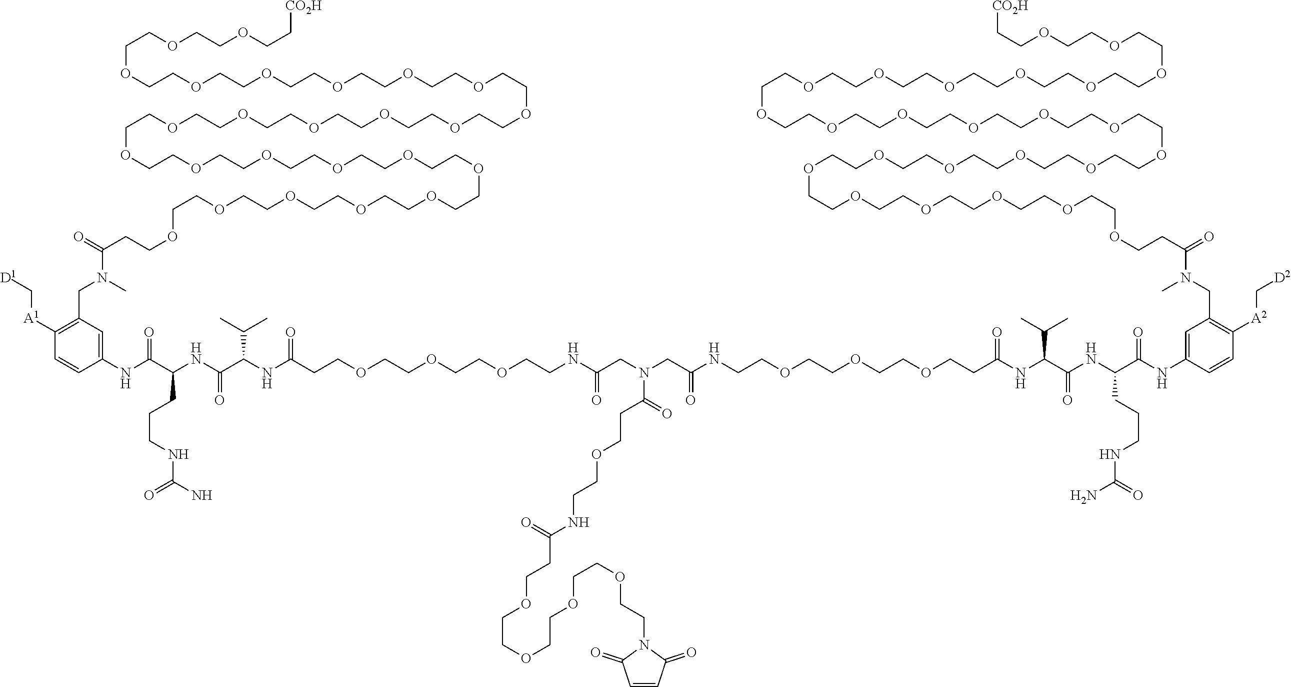

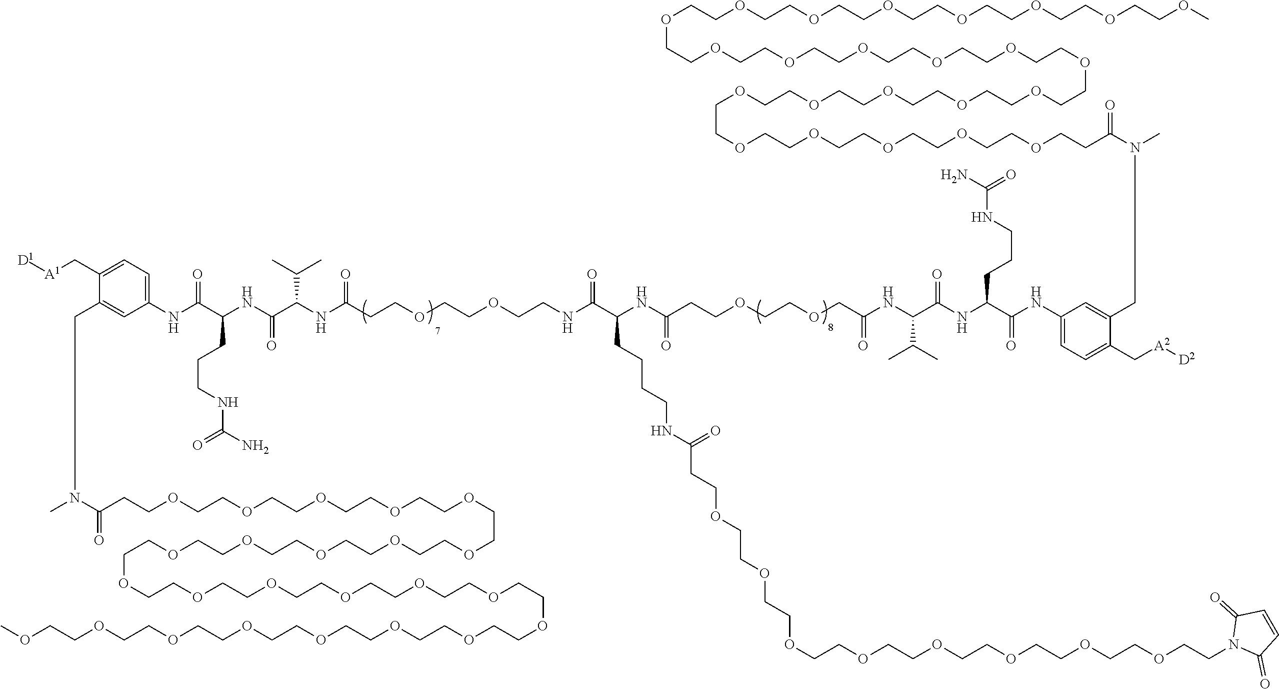

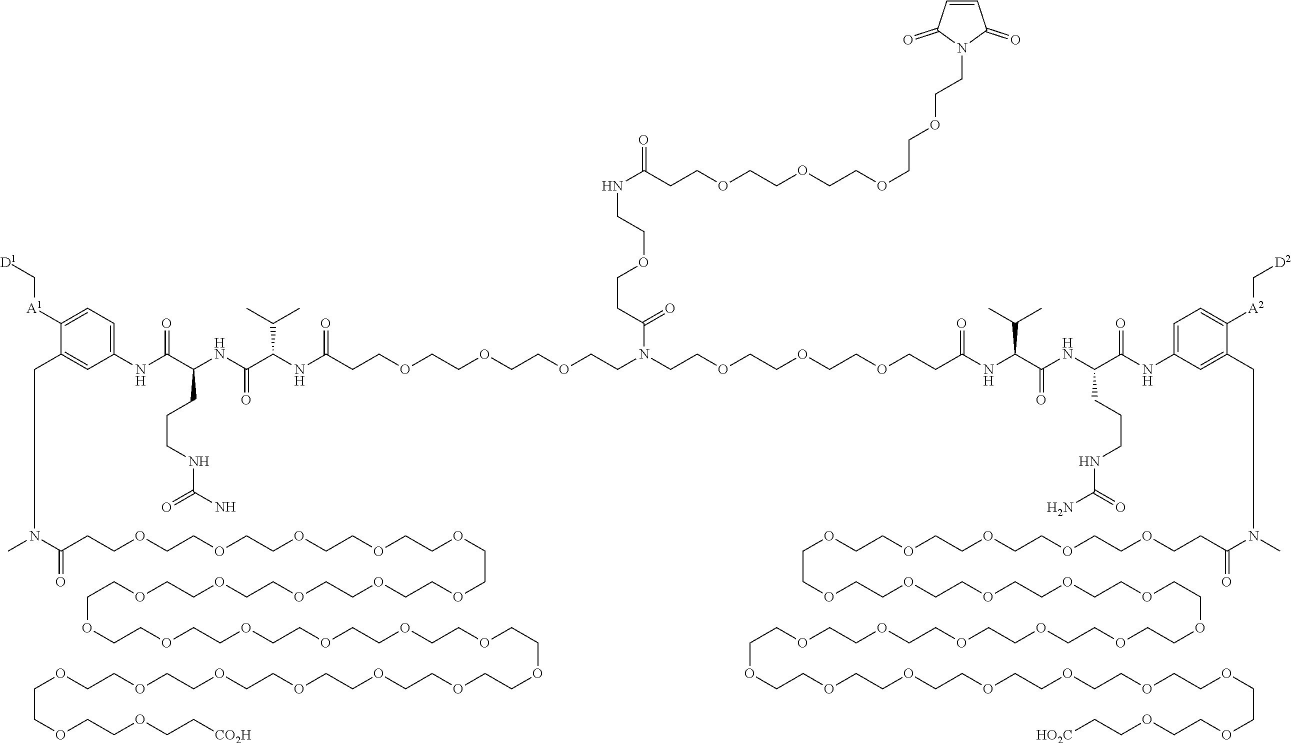

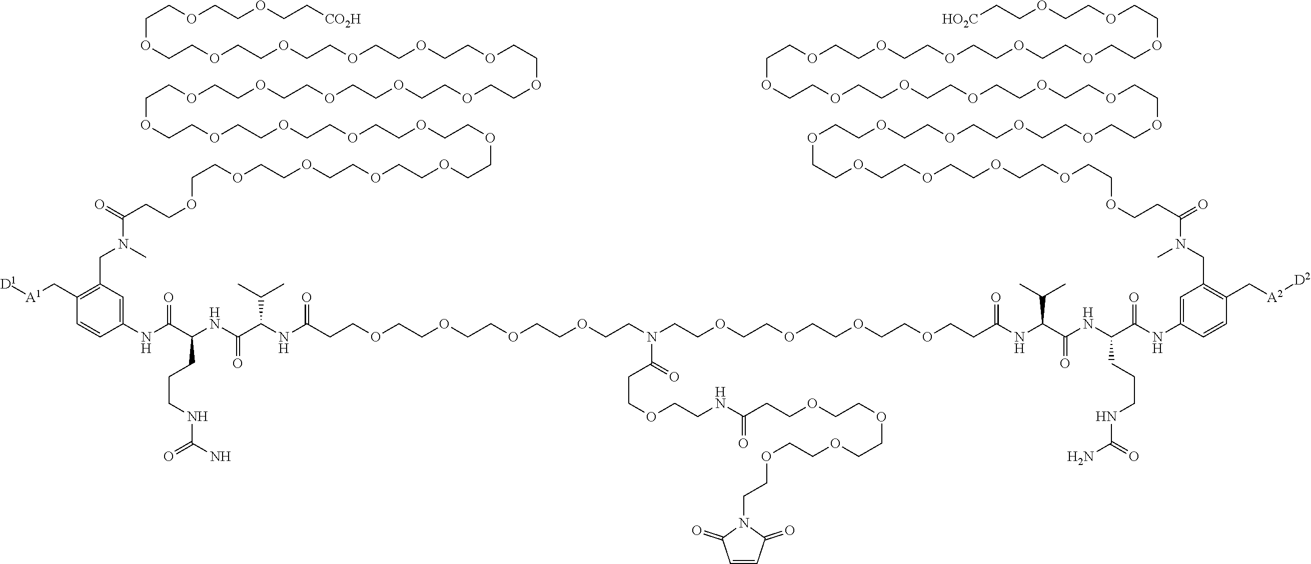

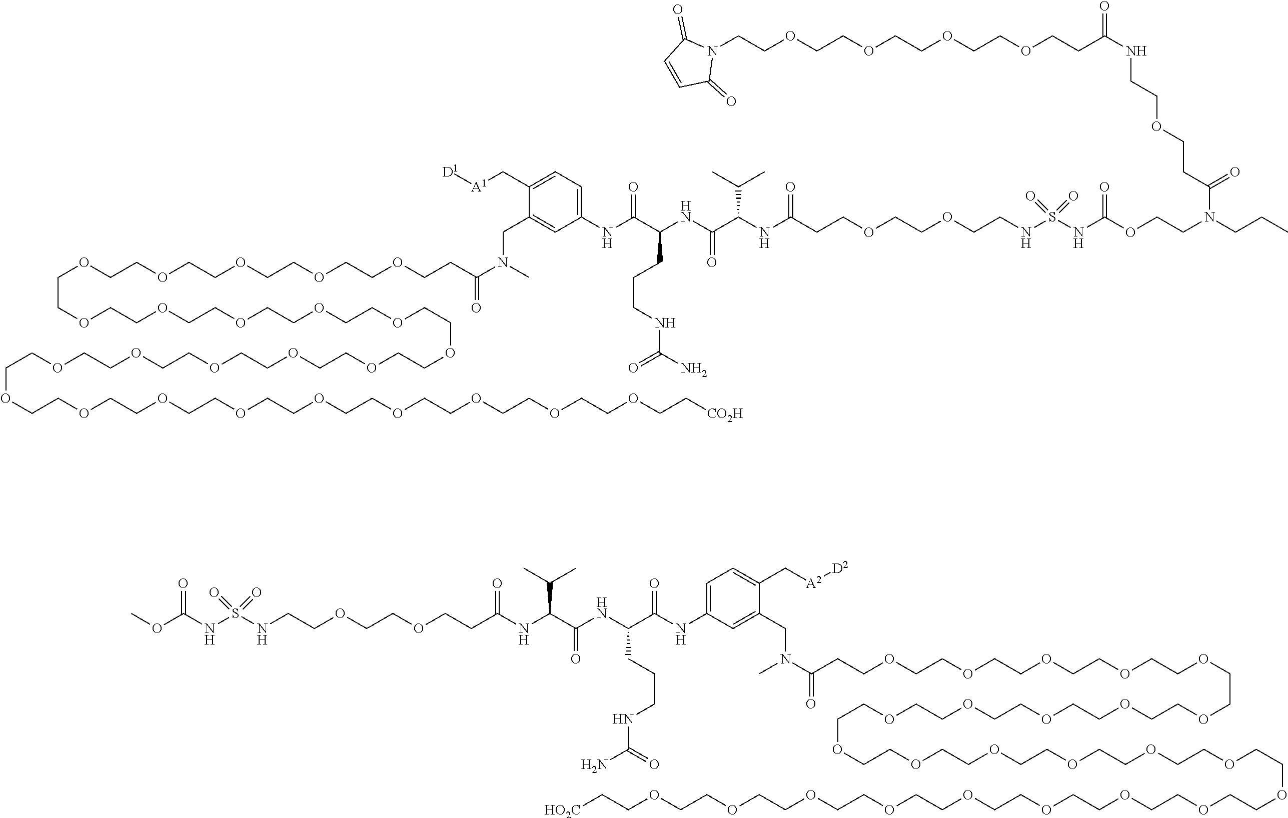

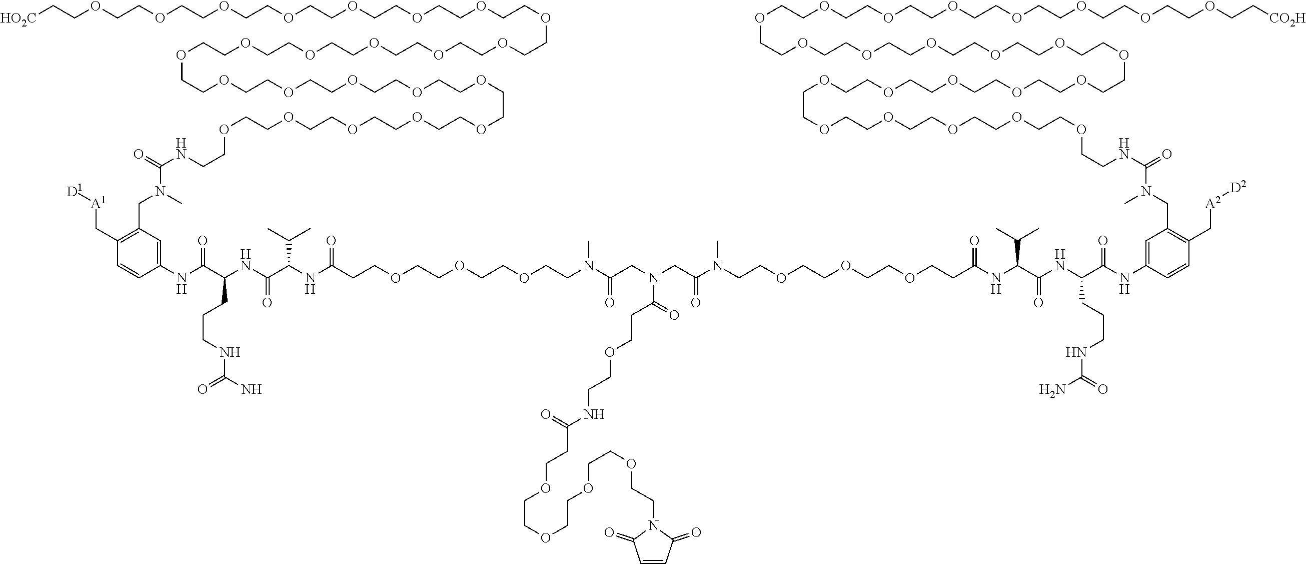

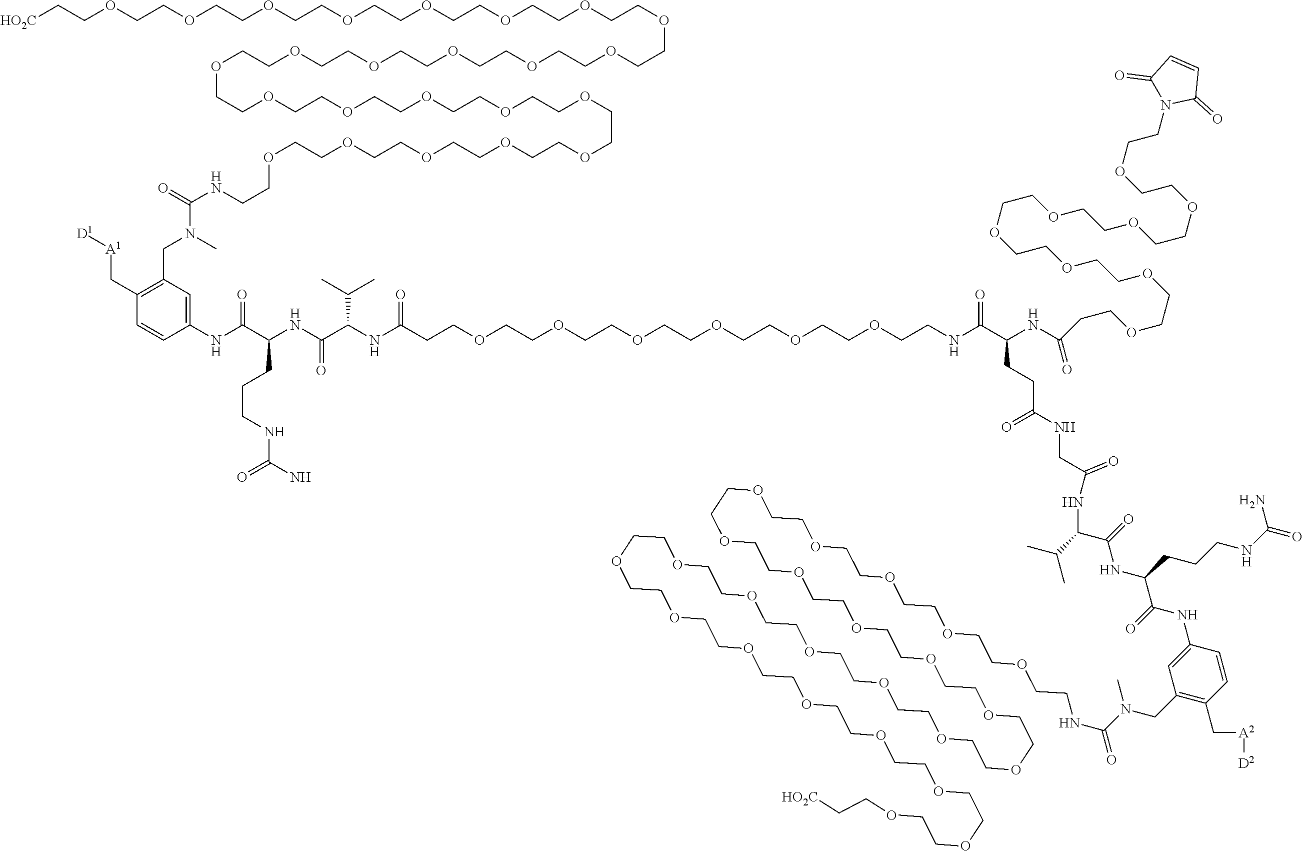

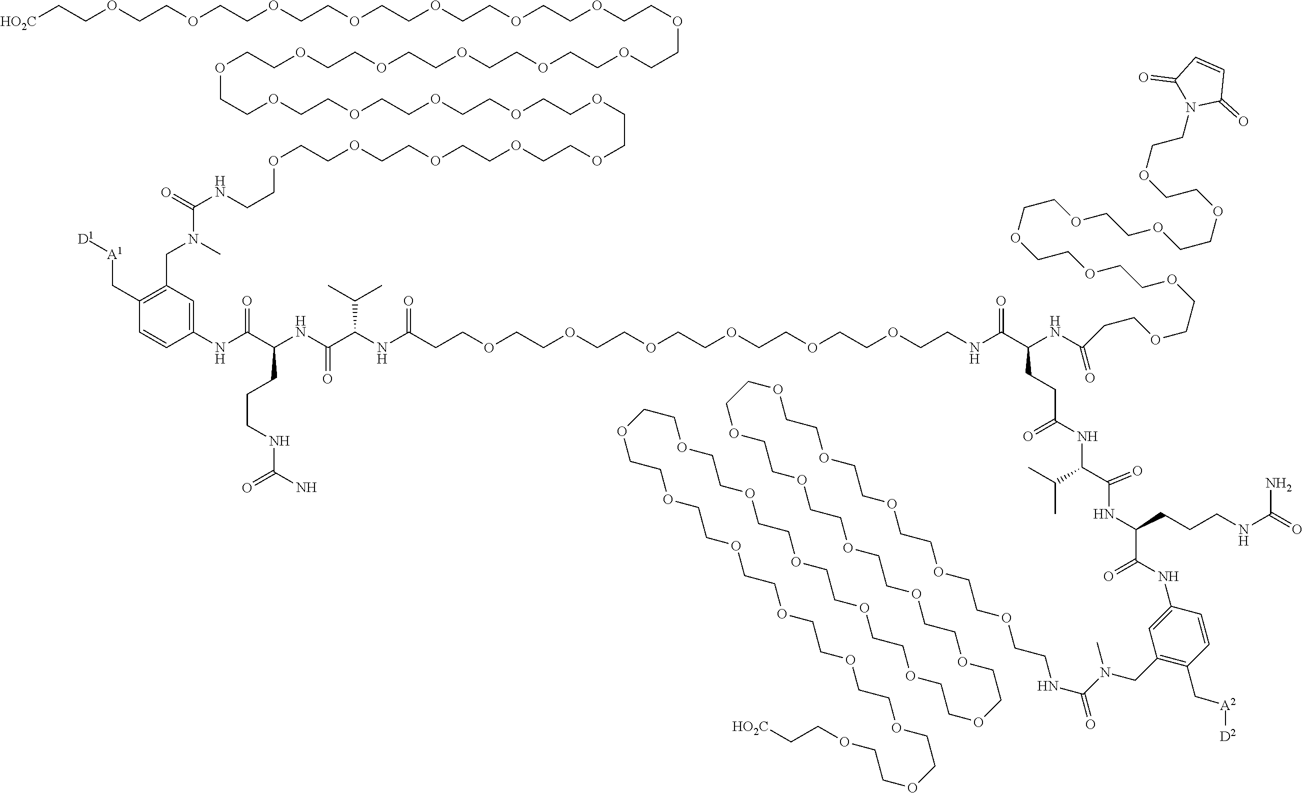

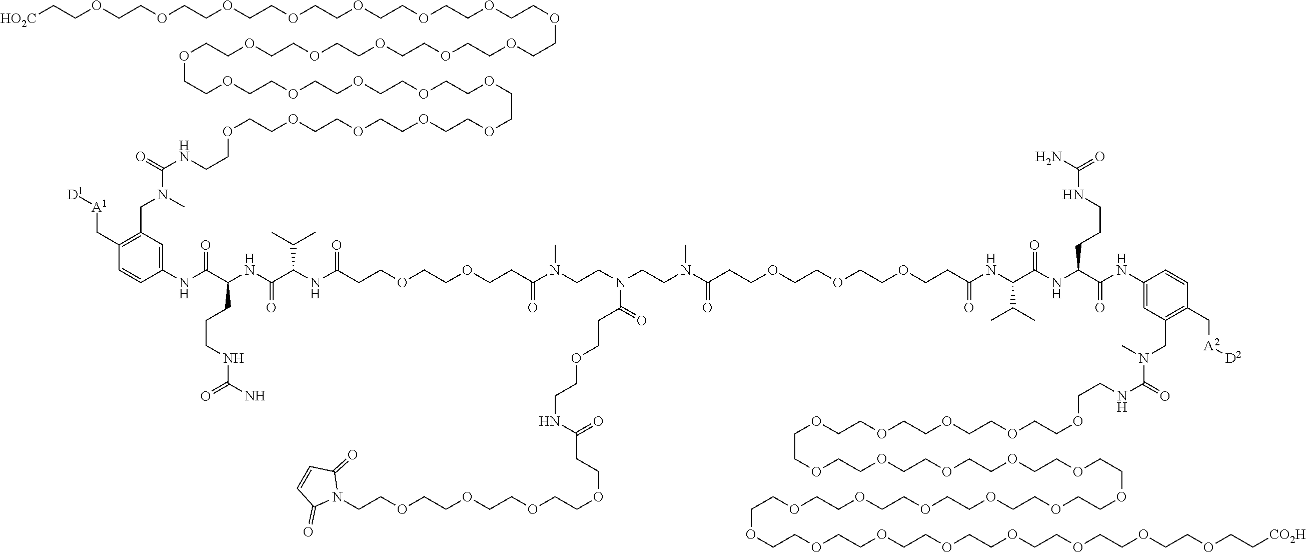

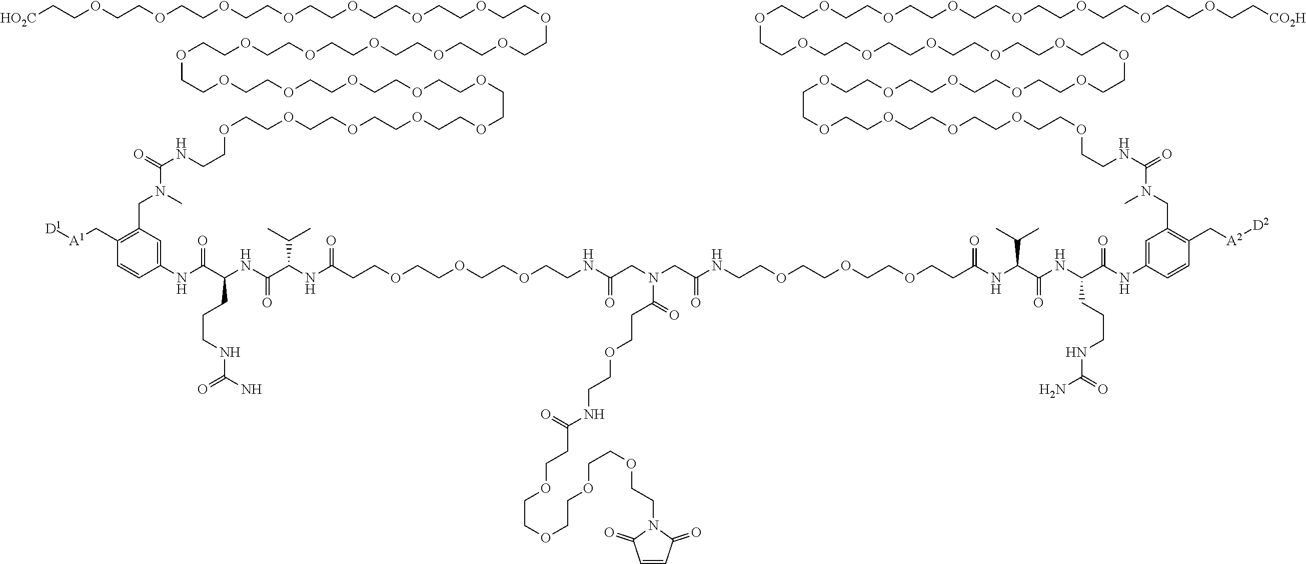

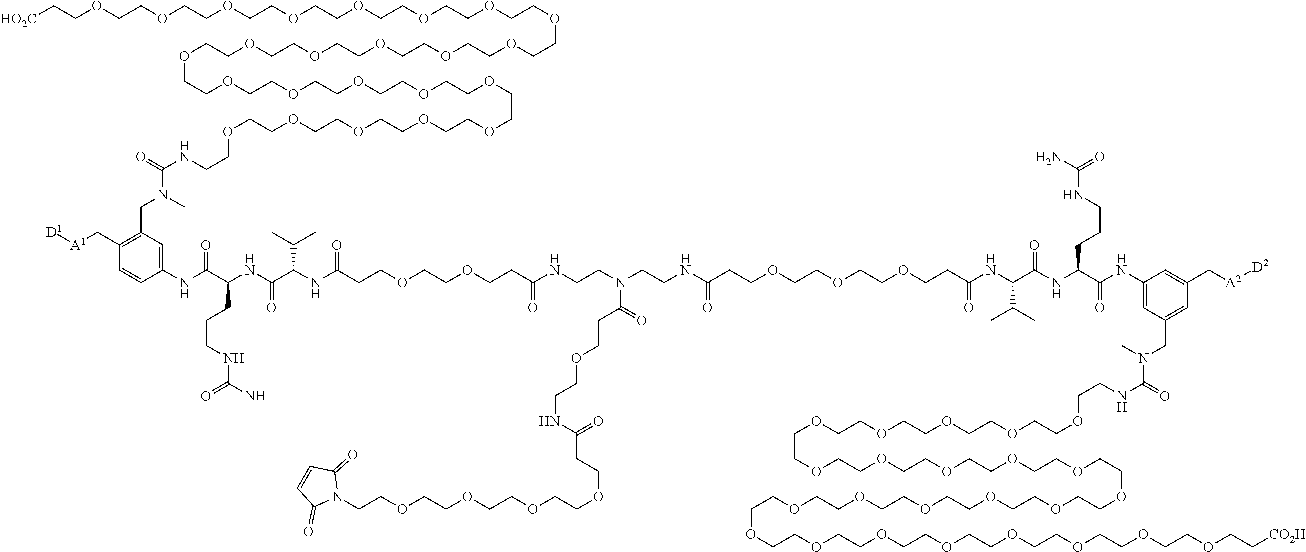

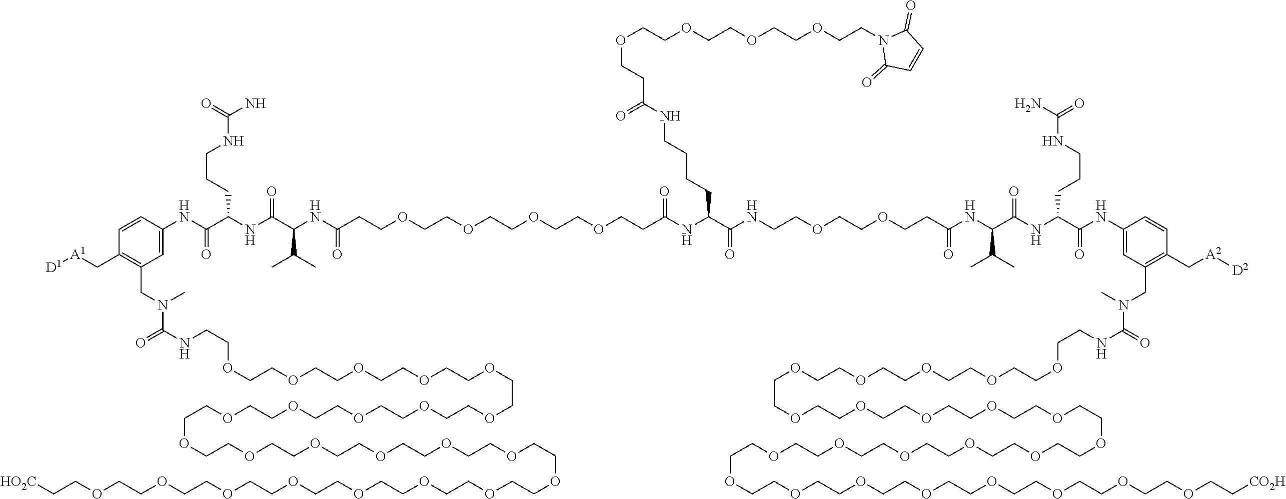

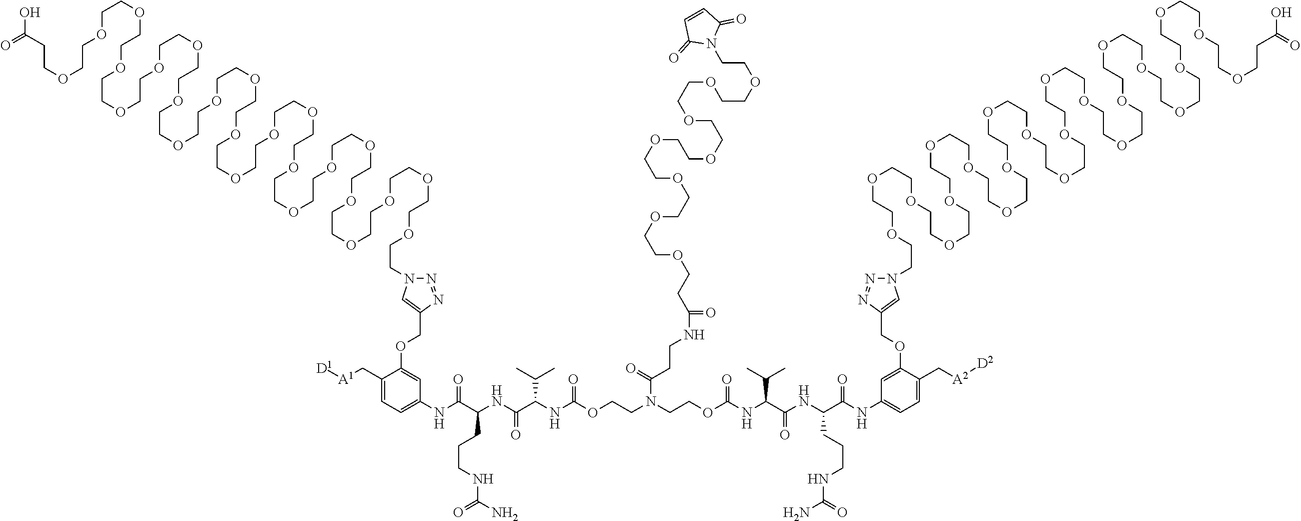

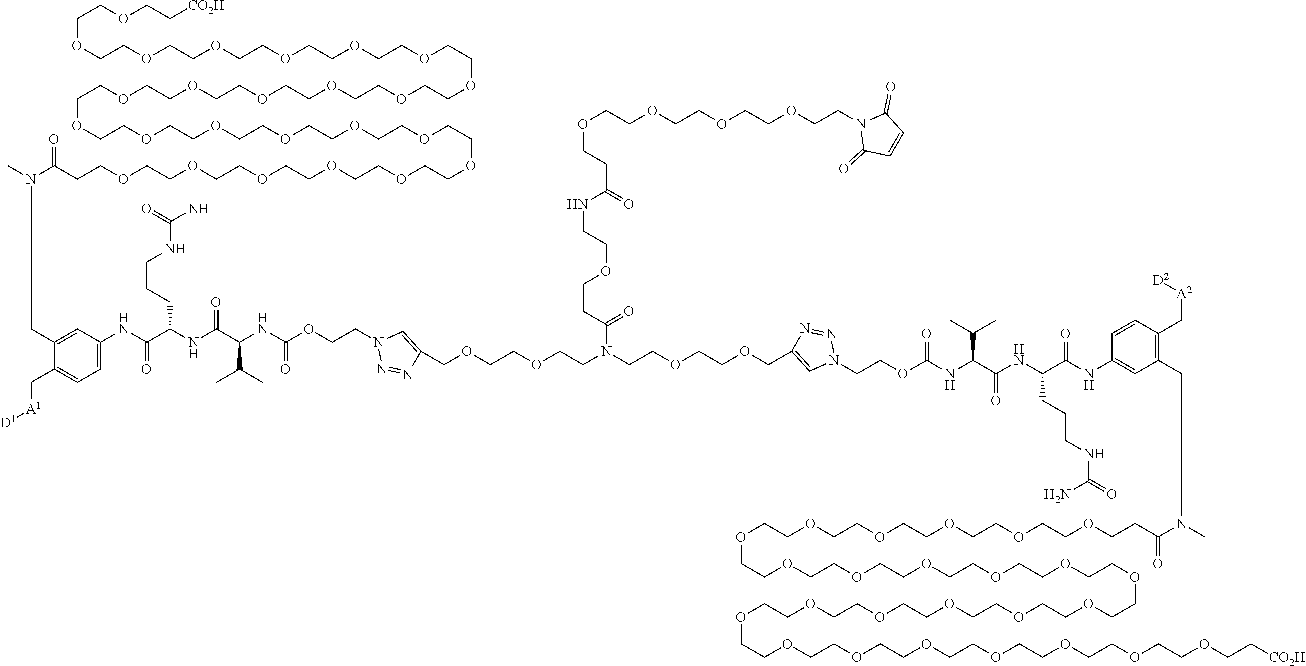

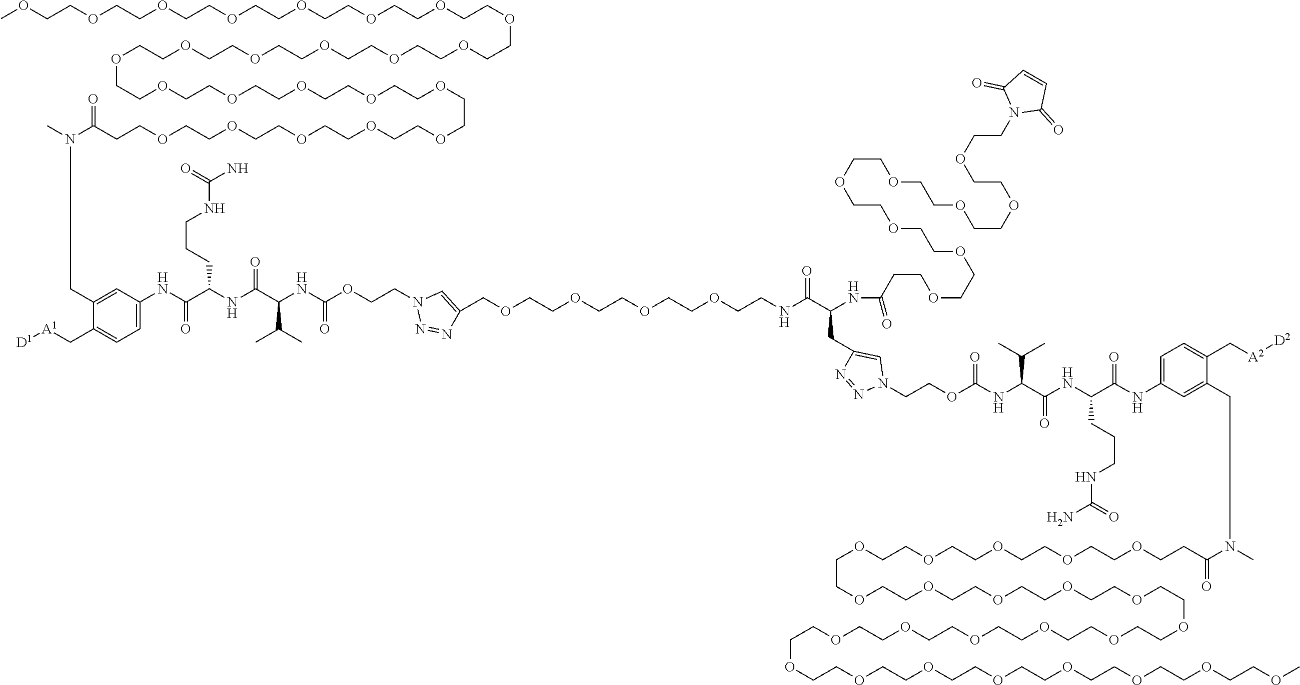

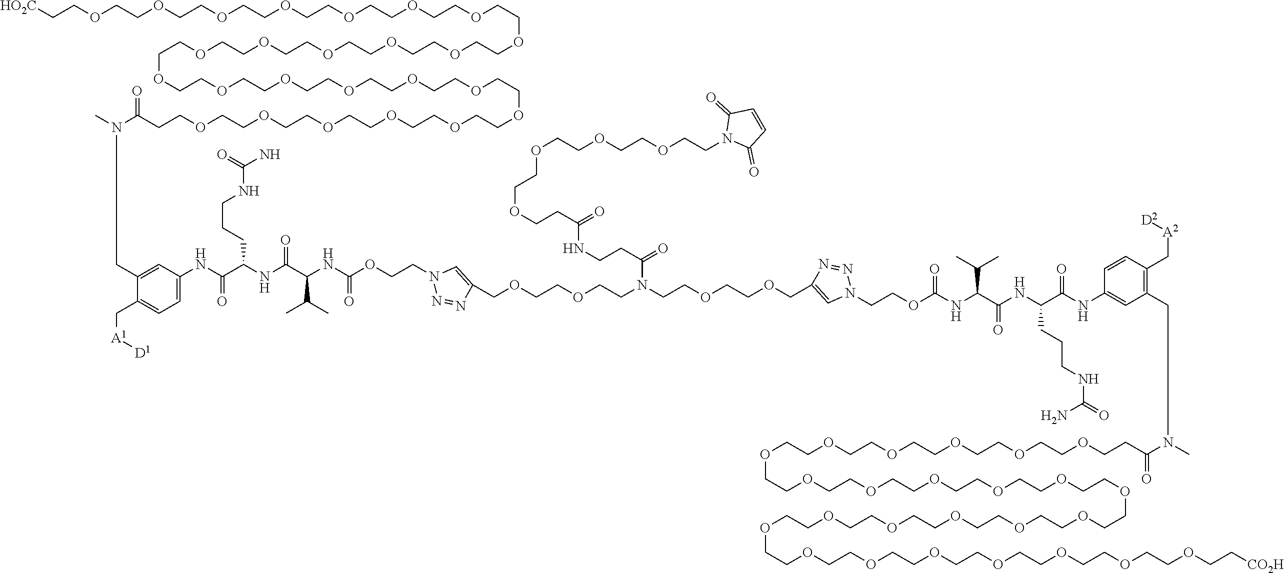

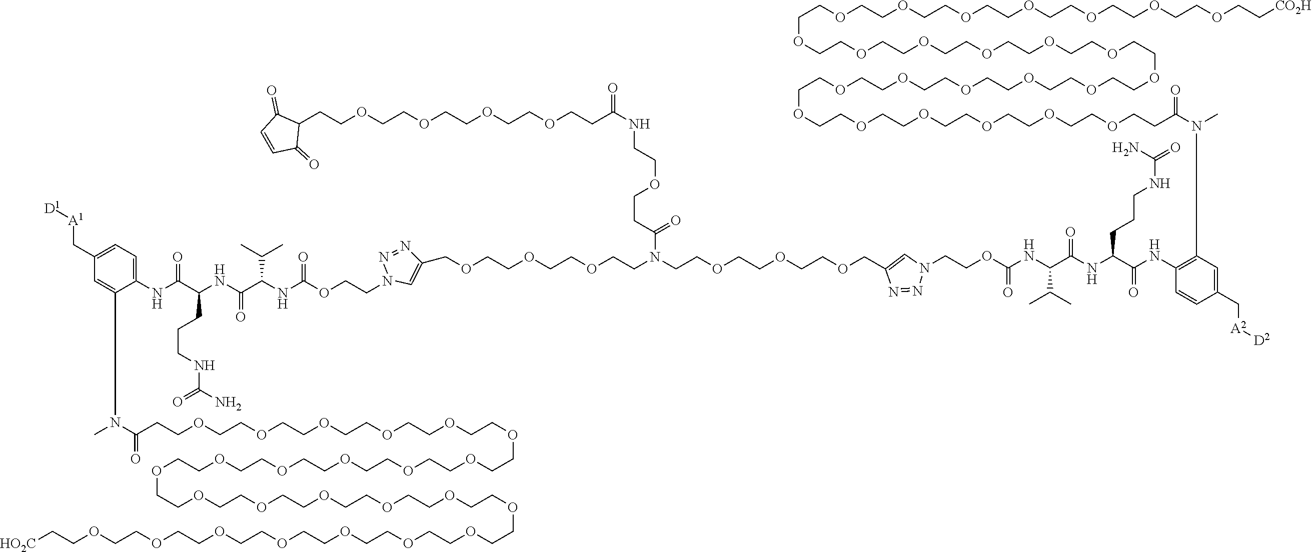

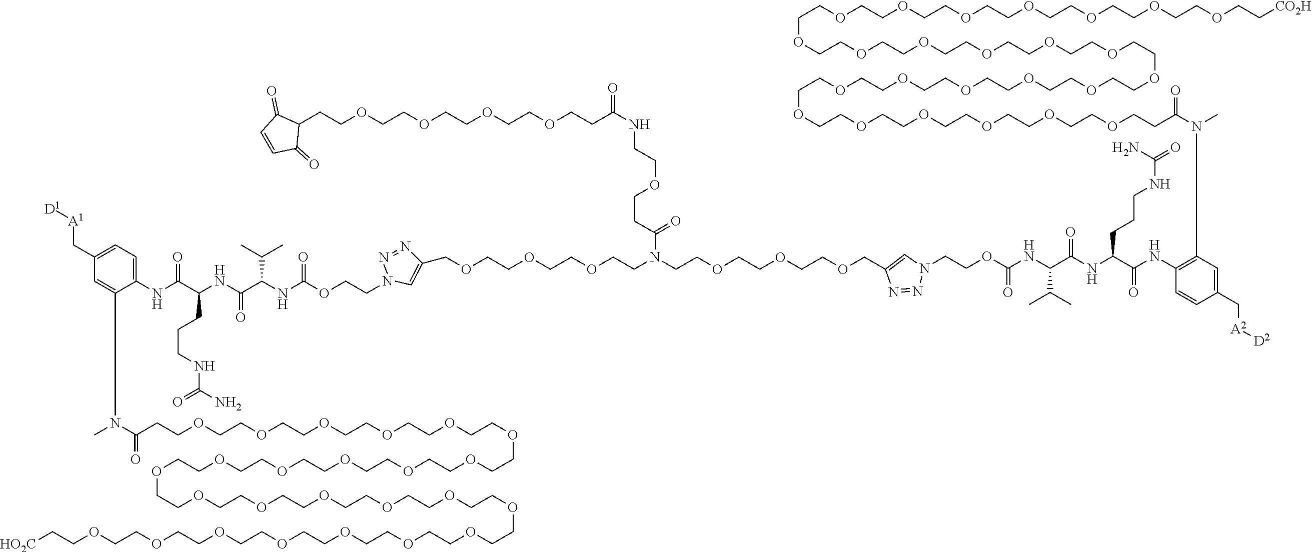

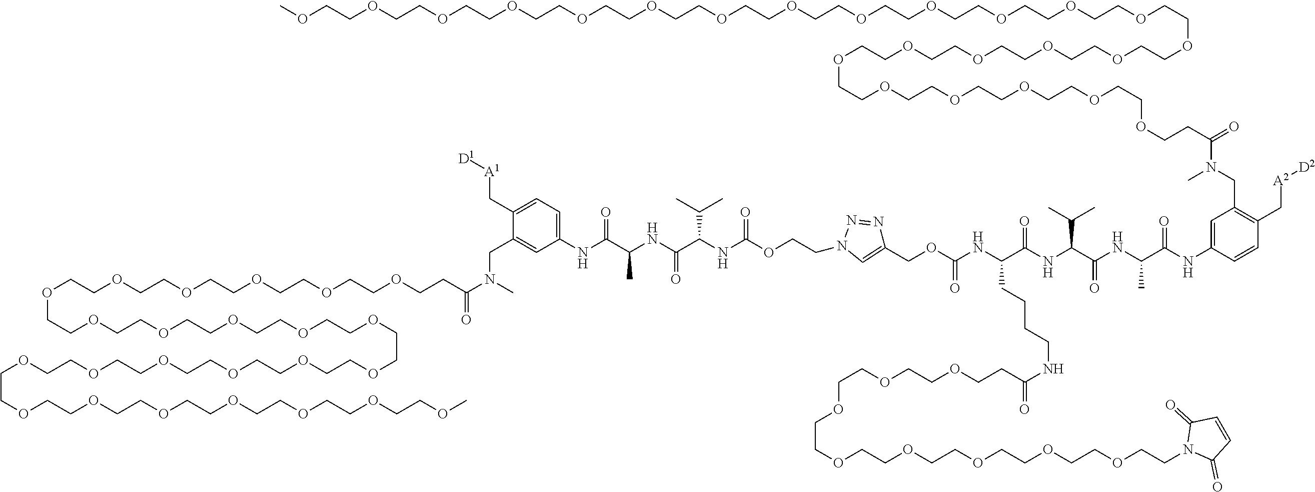

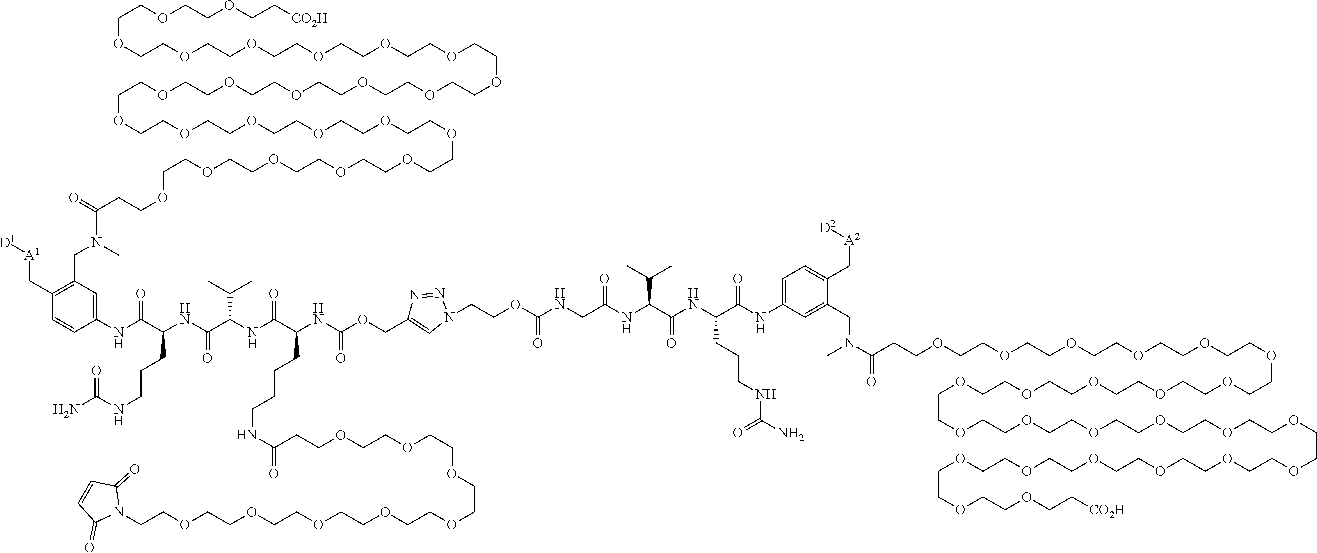

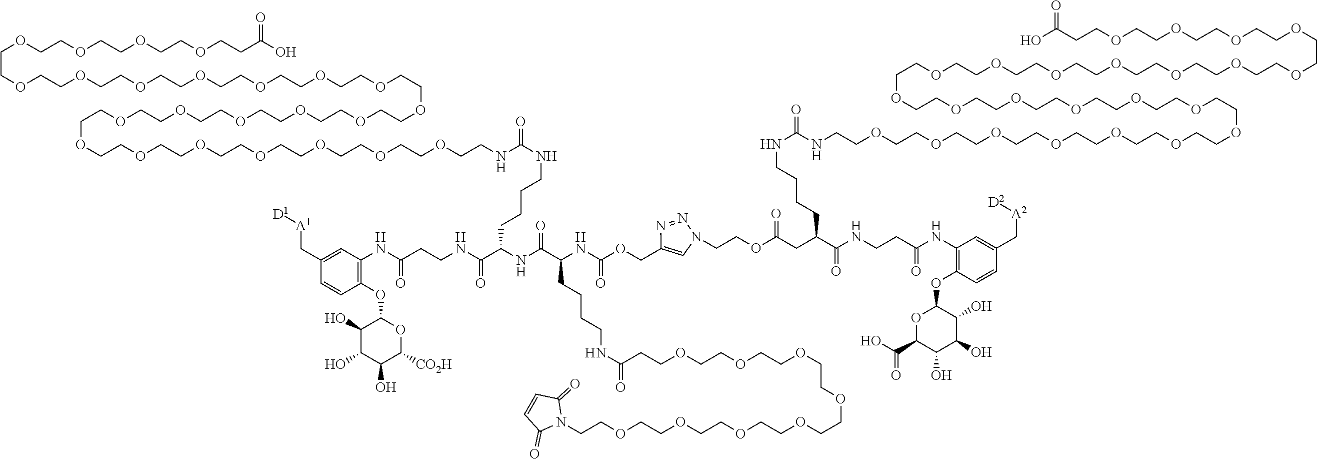

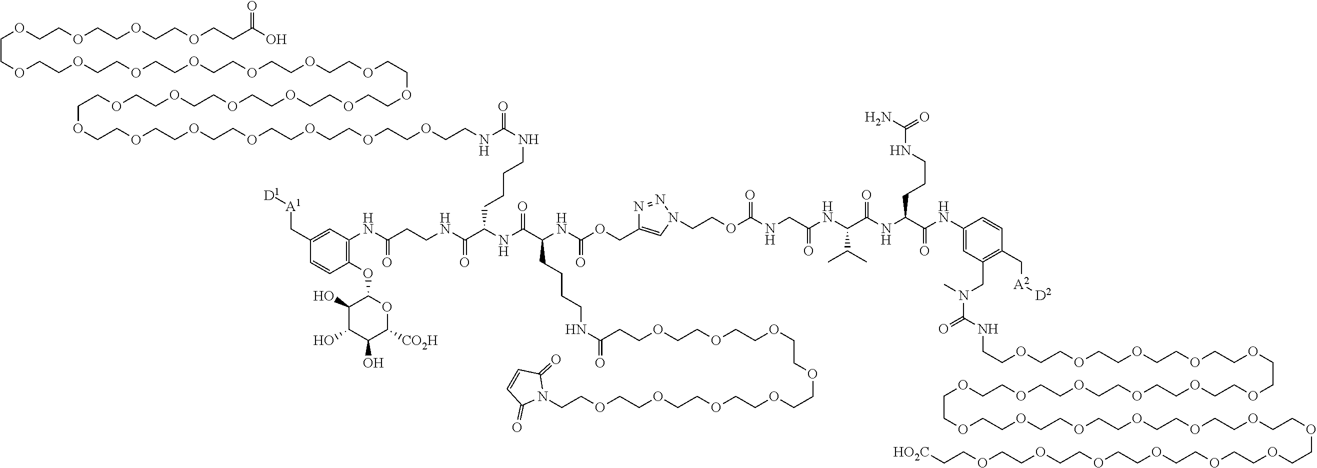

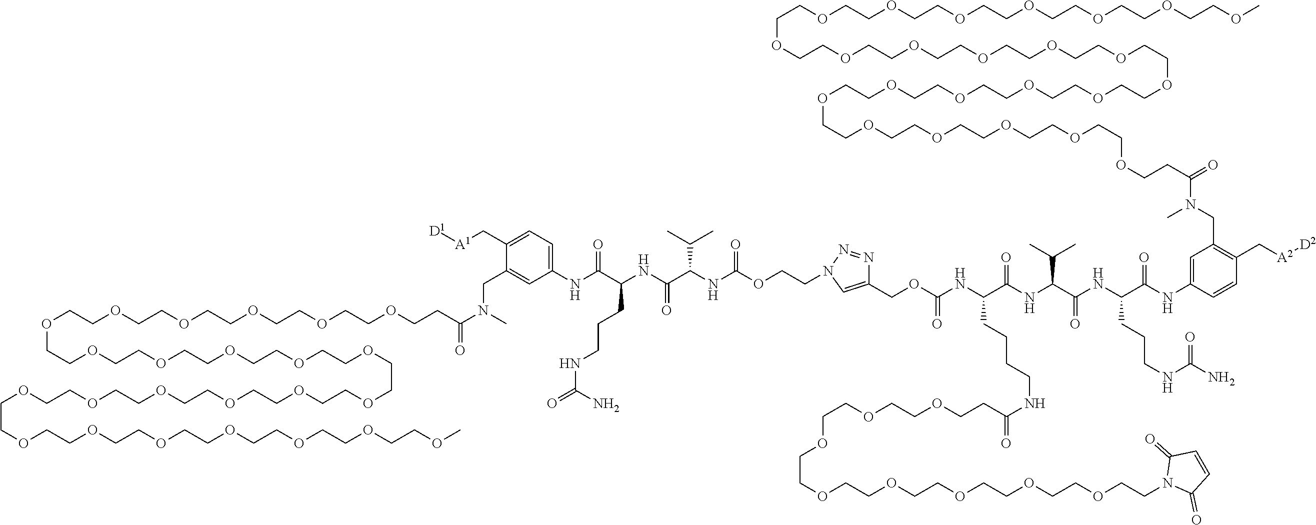

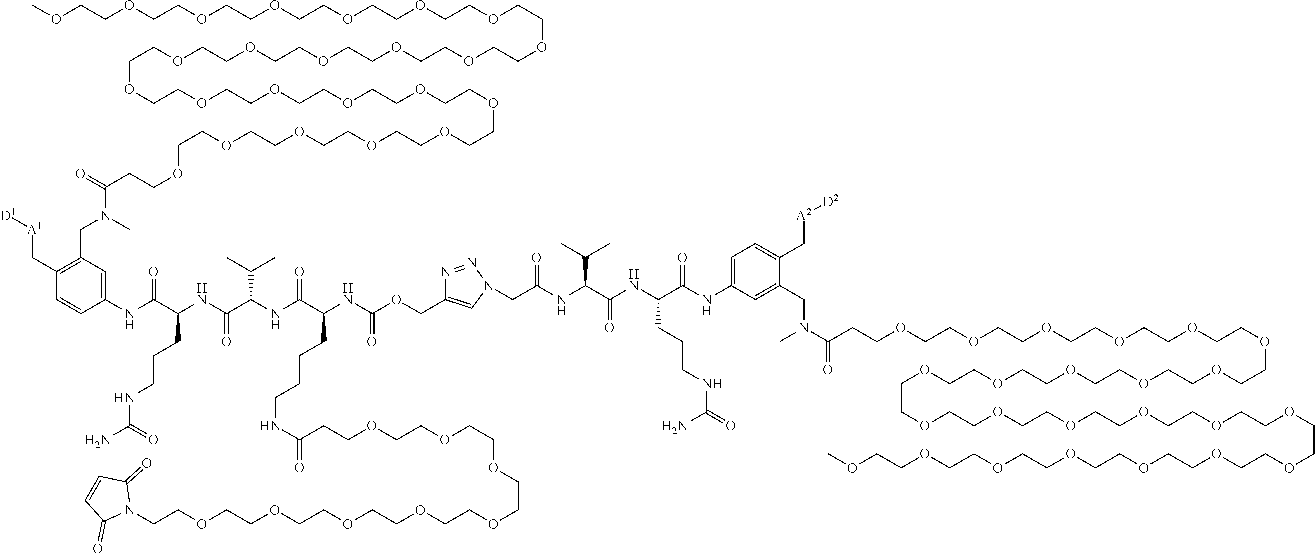

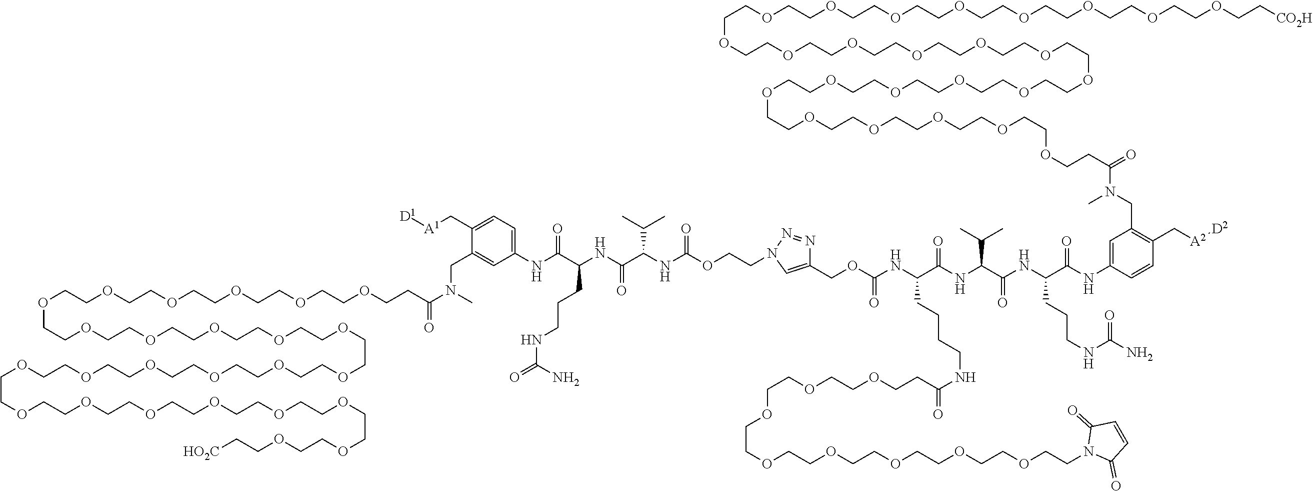

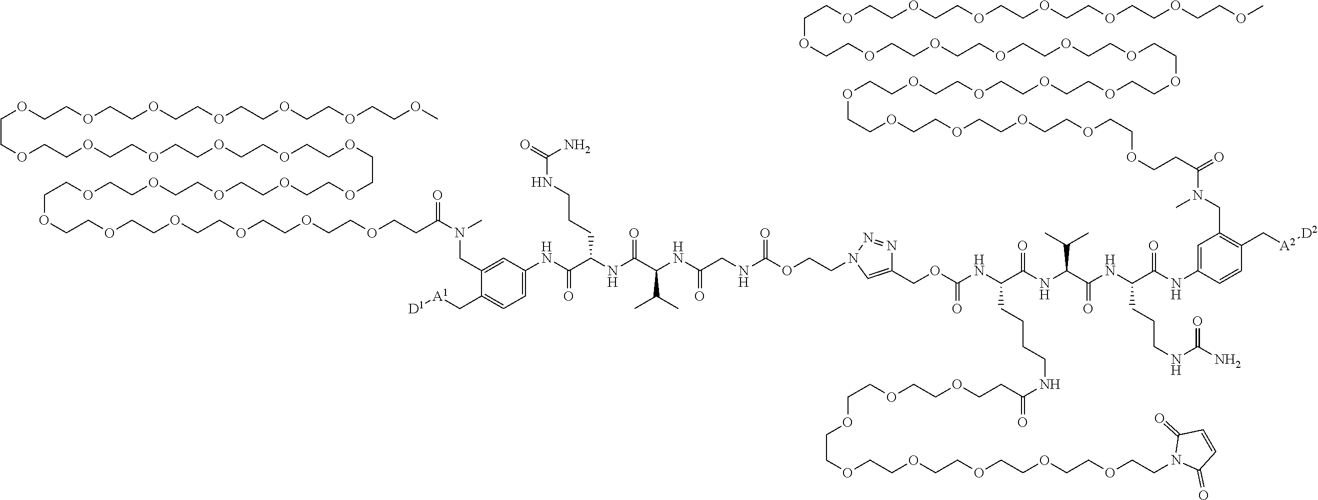

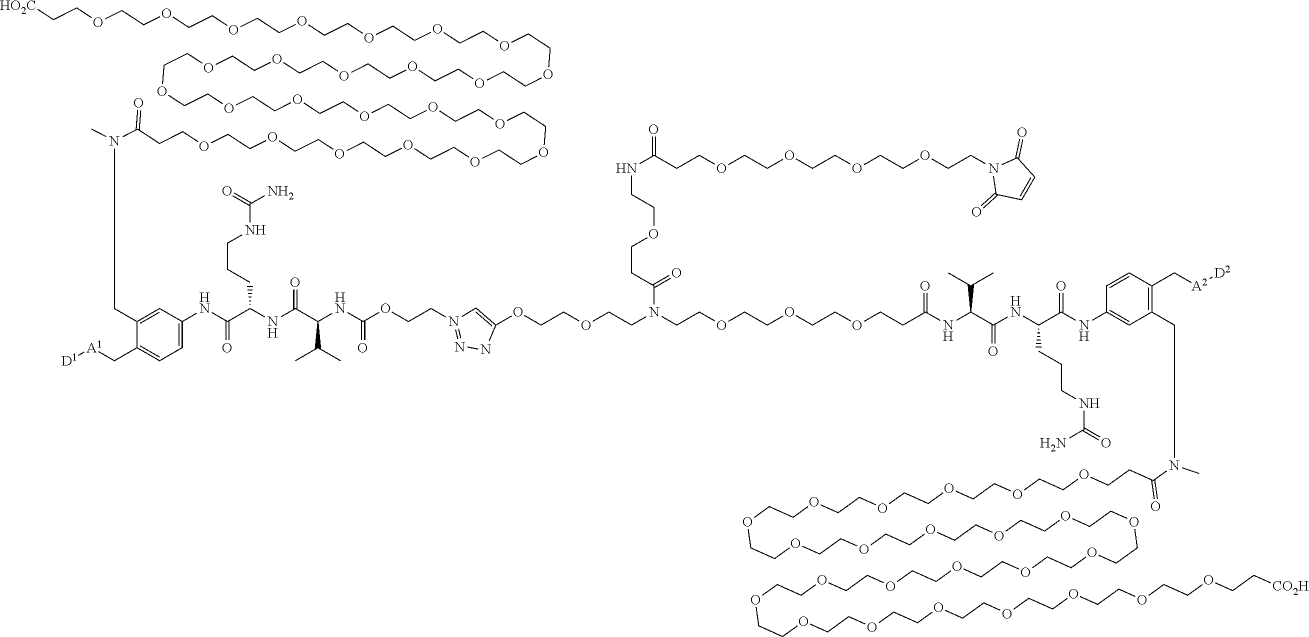

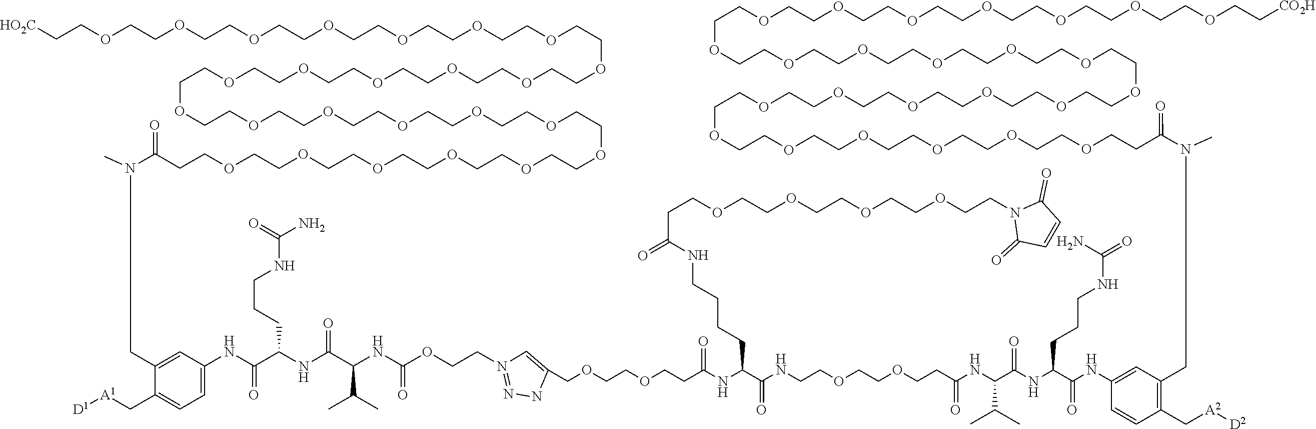

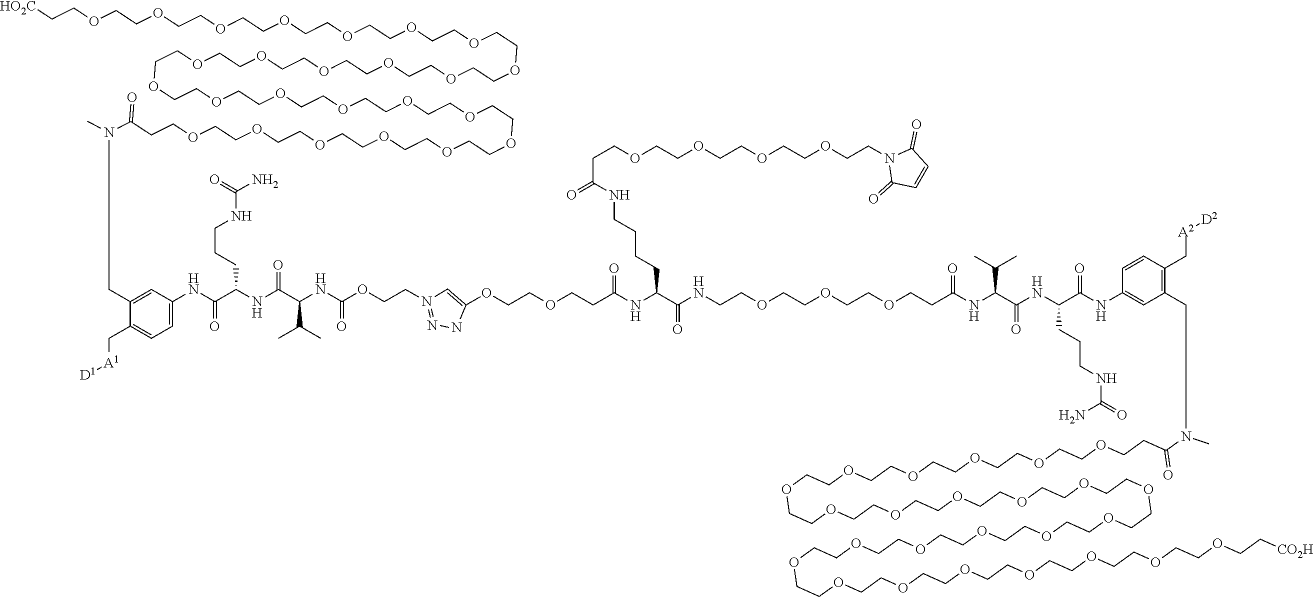

or a pharmaceutically acceptable salt thereof.
Embodiment 45. The compound or pharmaceutically acceptable salt thereof according to any one of the preceding Embodiments, selected from the group consisting of:

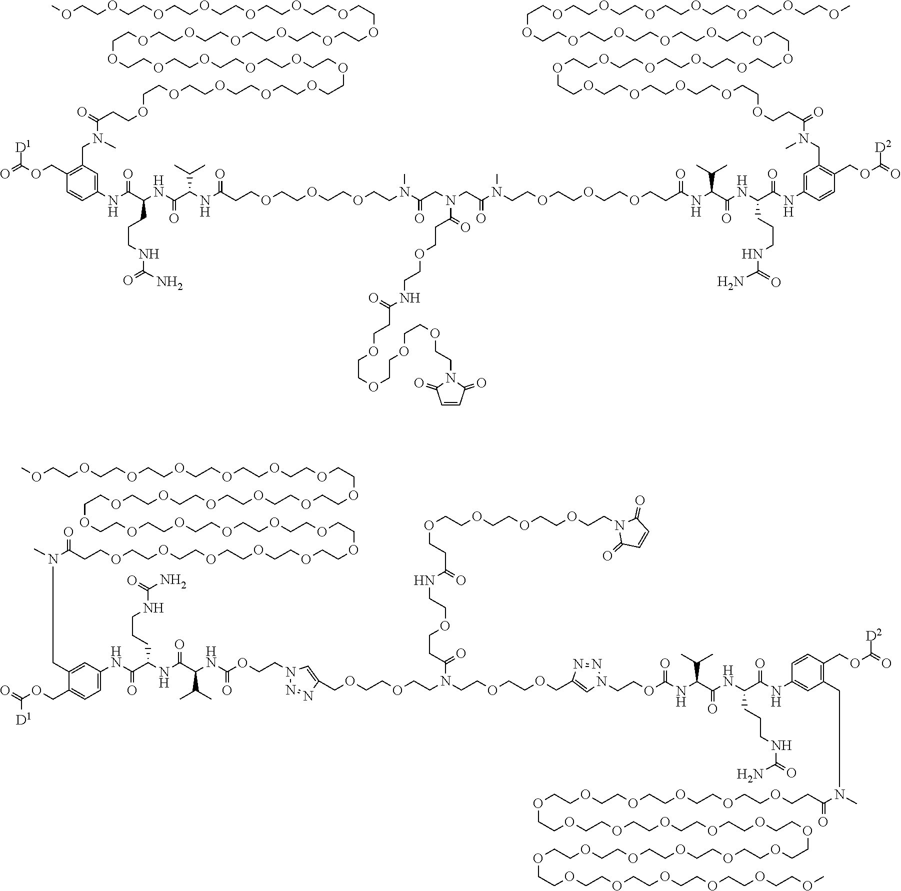

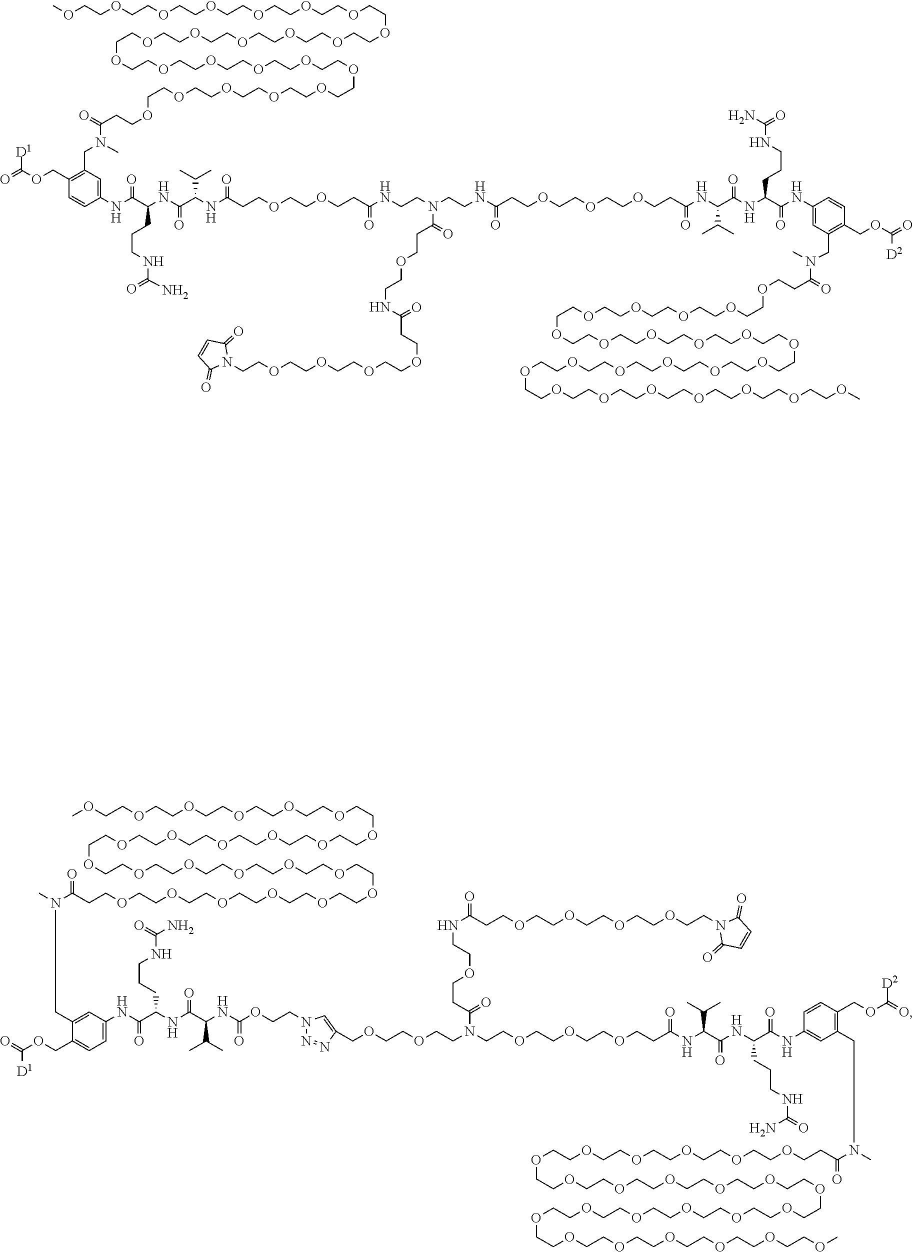

or a pharmaceutically acceptable salt thereof.
Embodiment 45a. The compound or pharmaceutically acceptable salt thereof according to any one of Embodiments 1 to 45, selected from the group consisting of:

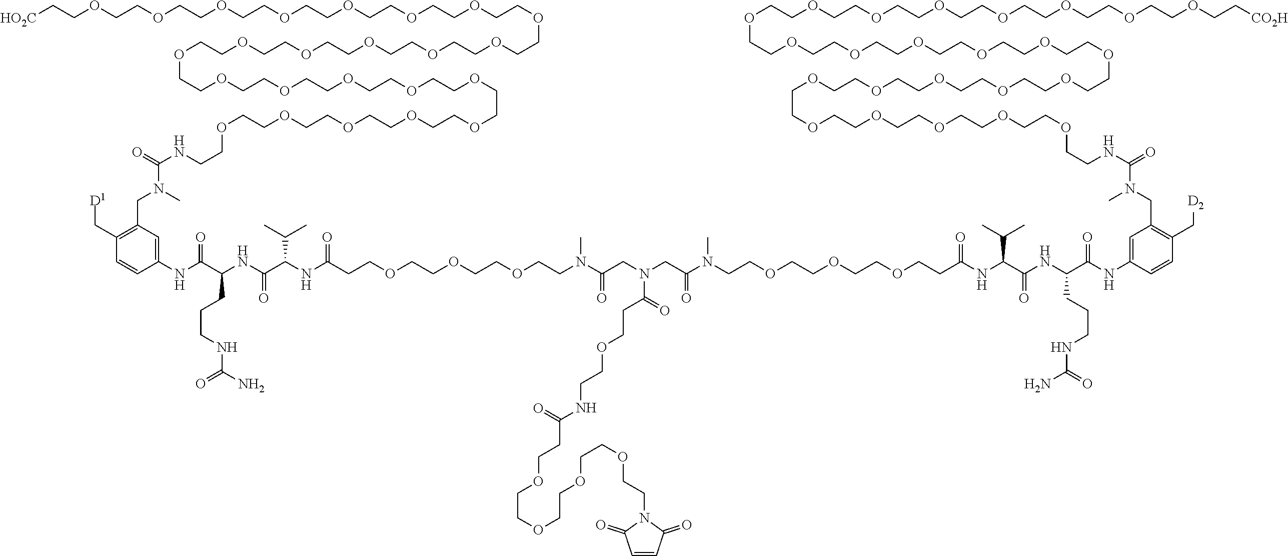

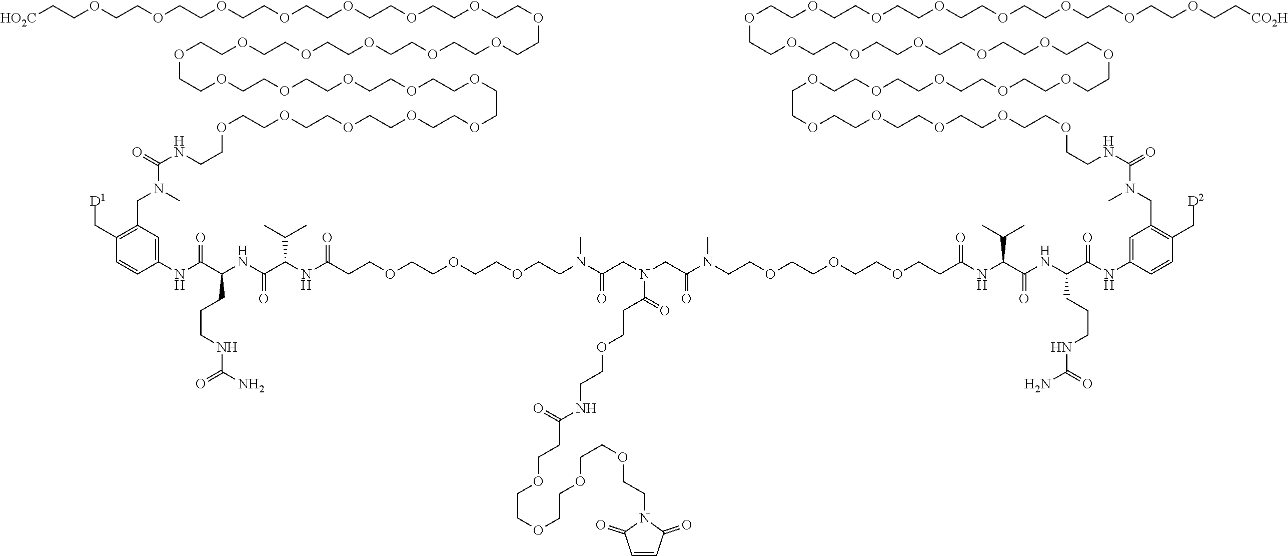

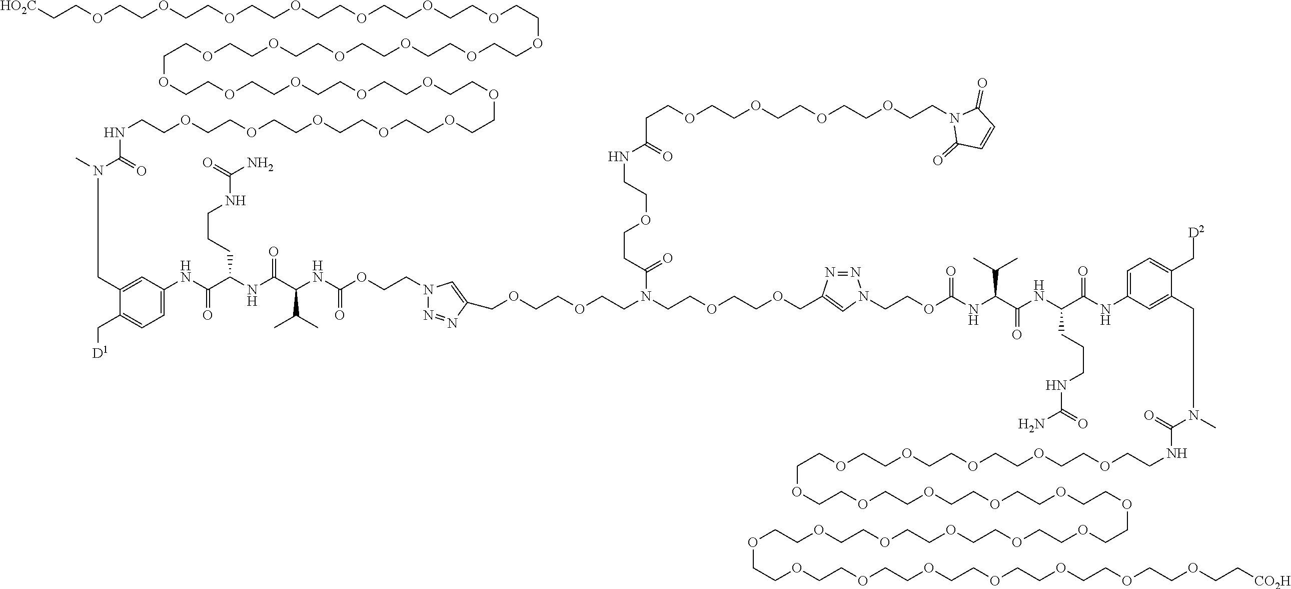

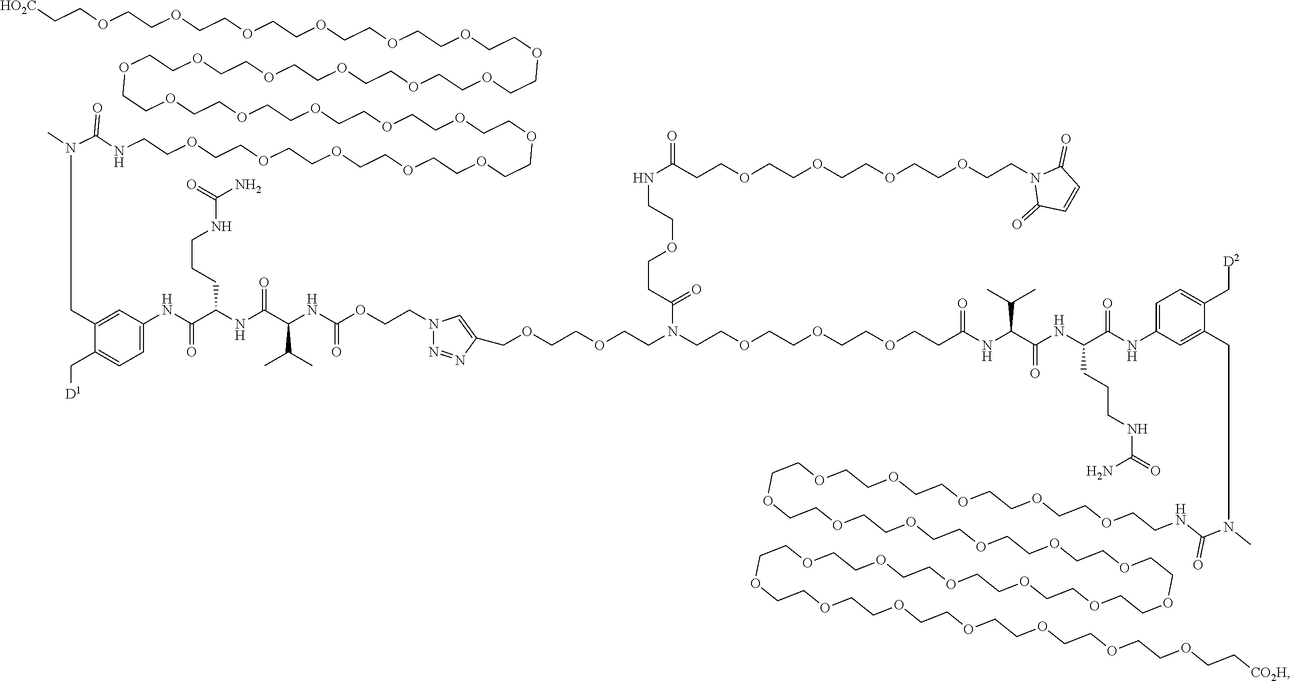

or a pharmaceutically acceptable salt thereof.
Embodiment 45b. The compound or pharmaceutically acceptable salt thereof according to Embodiment 45a, selected from the group consisting of:

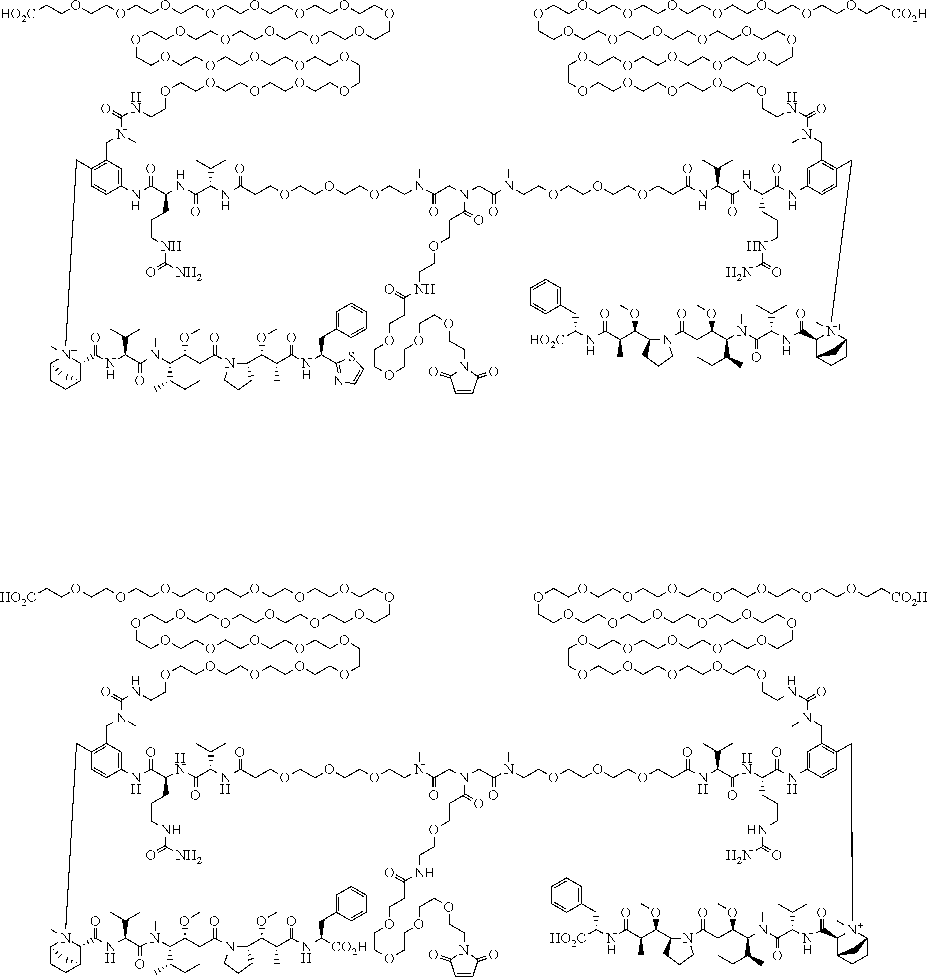

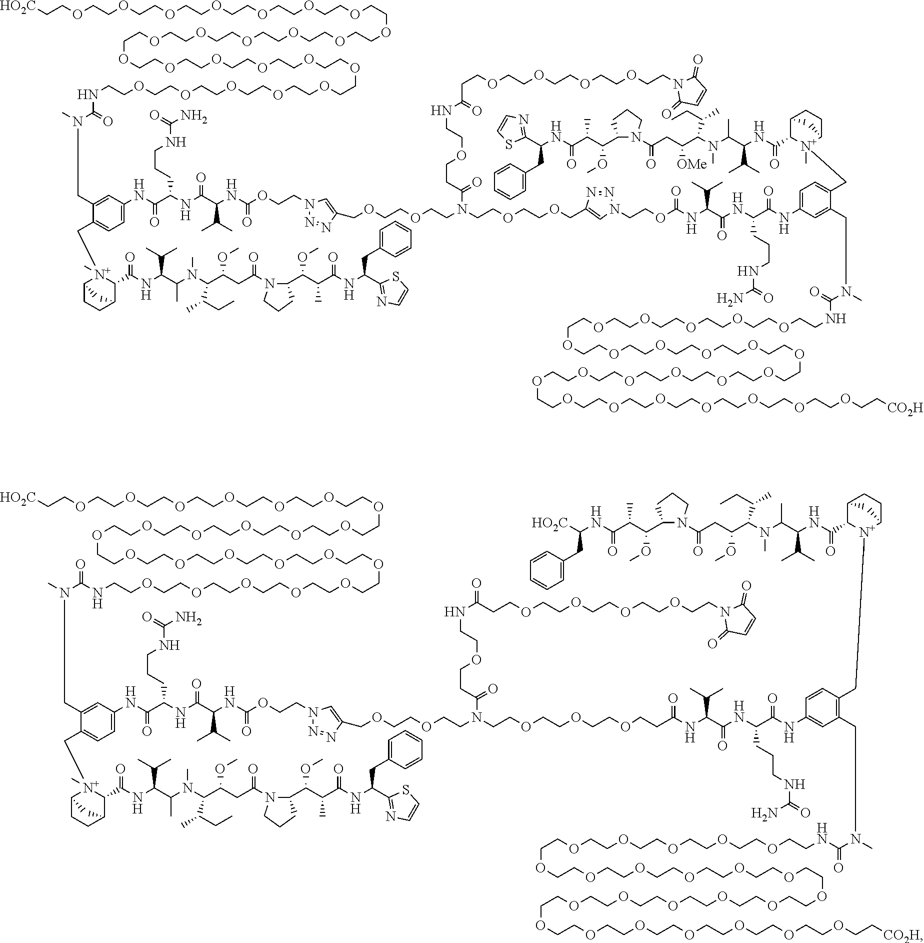

or a pharmaceutically acceptable salt thereof.
Embodiment 45c. The compound or pharmaceutically acceptable salt thereof according to any one of Embodiments 1 to 43, selected from:

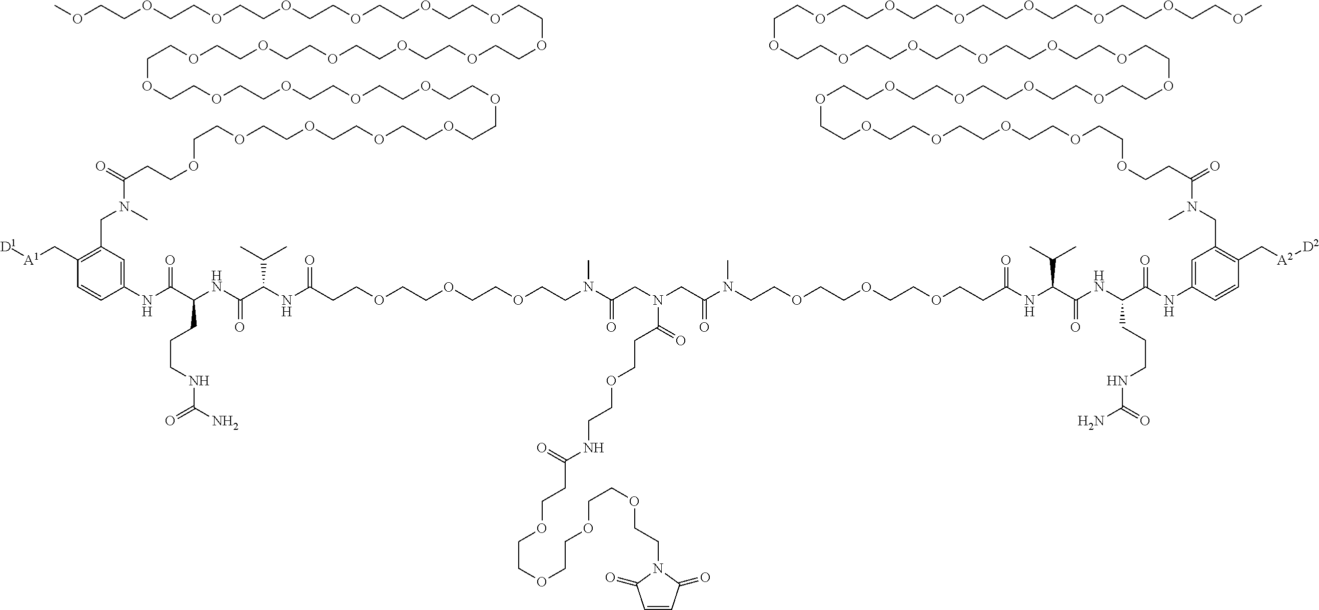

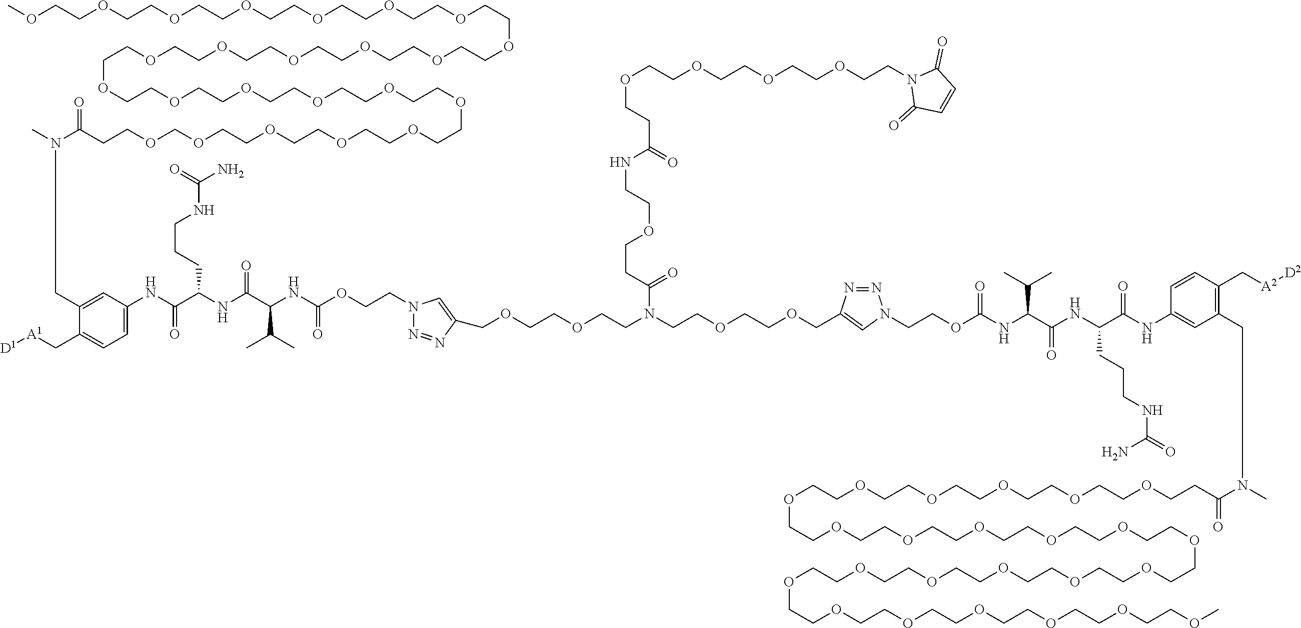

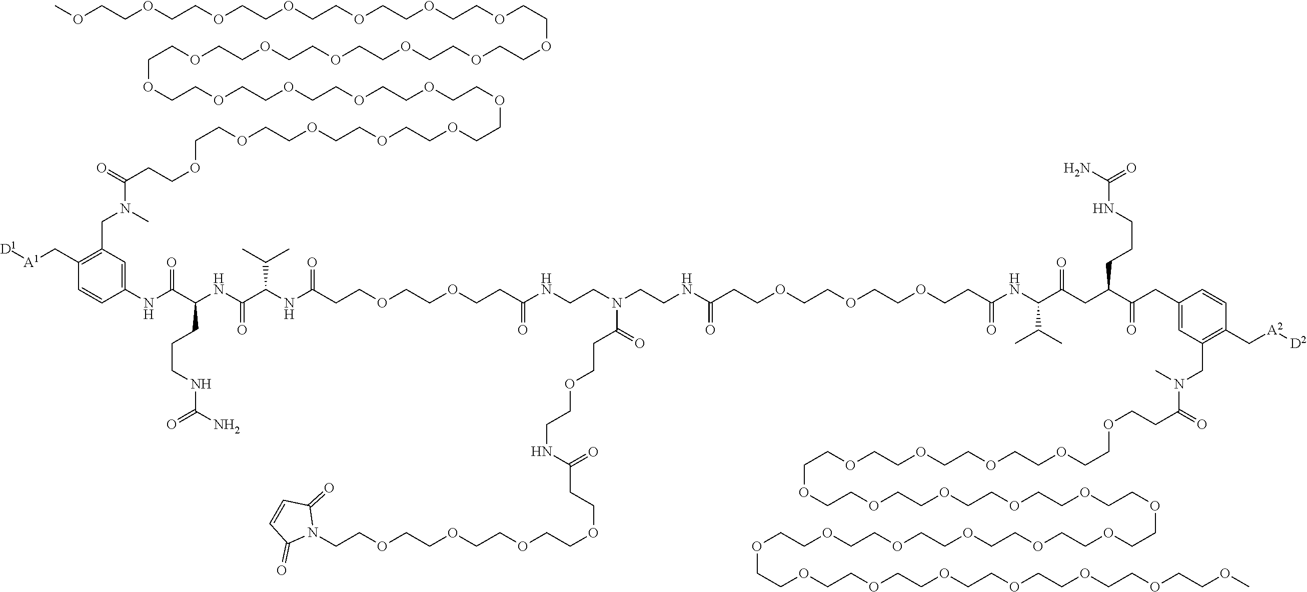

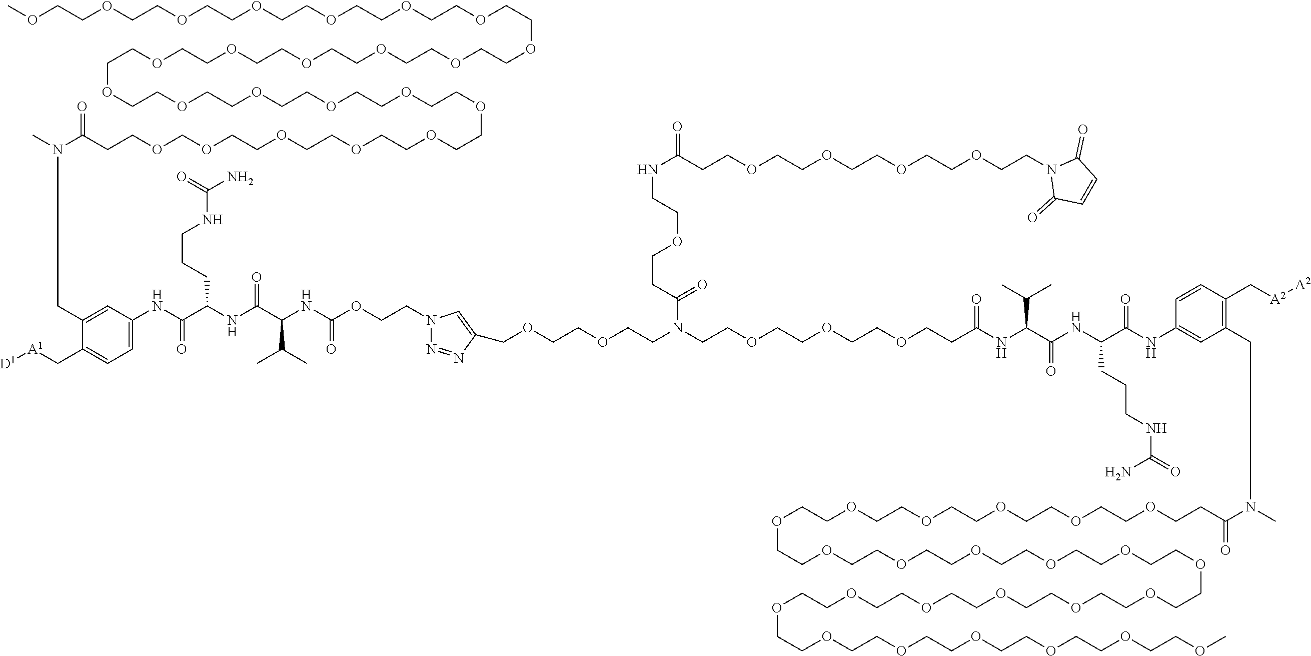

or a pharmaceutically acceptable salt thereof.
Embodiment 45d. The compound or pharmaceutically acceptable salt thereof according to any one of Embodiments 1 to 43, selected from:

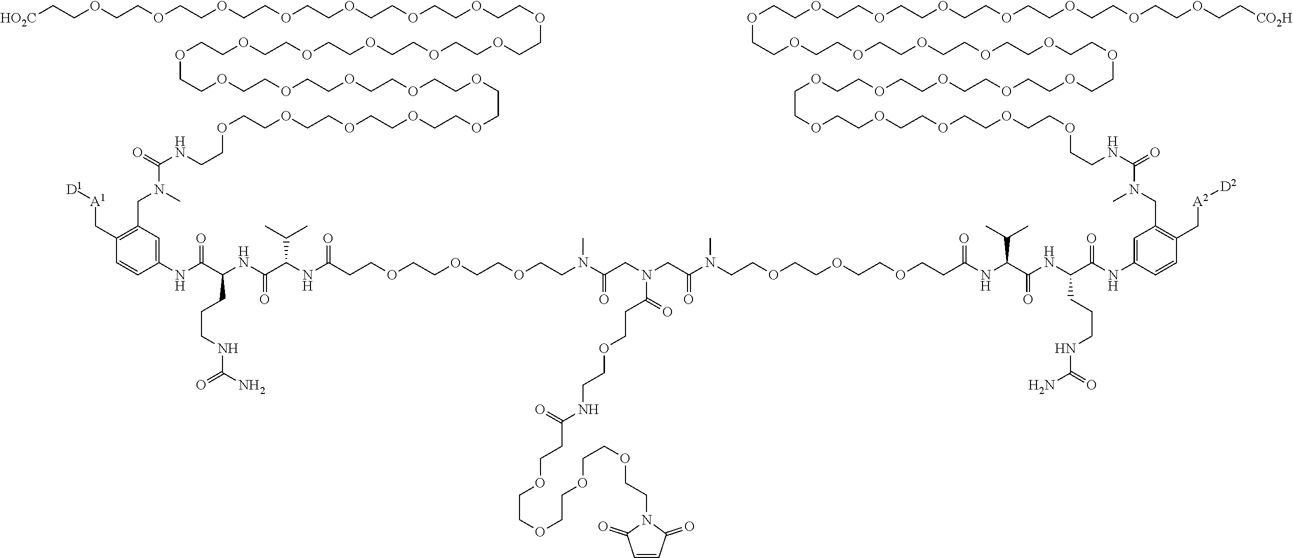

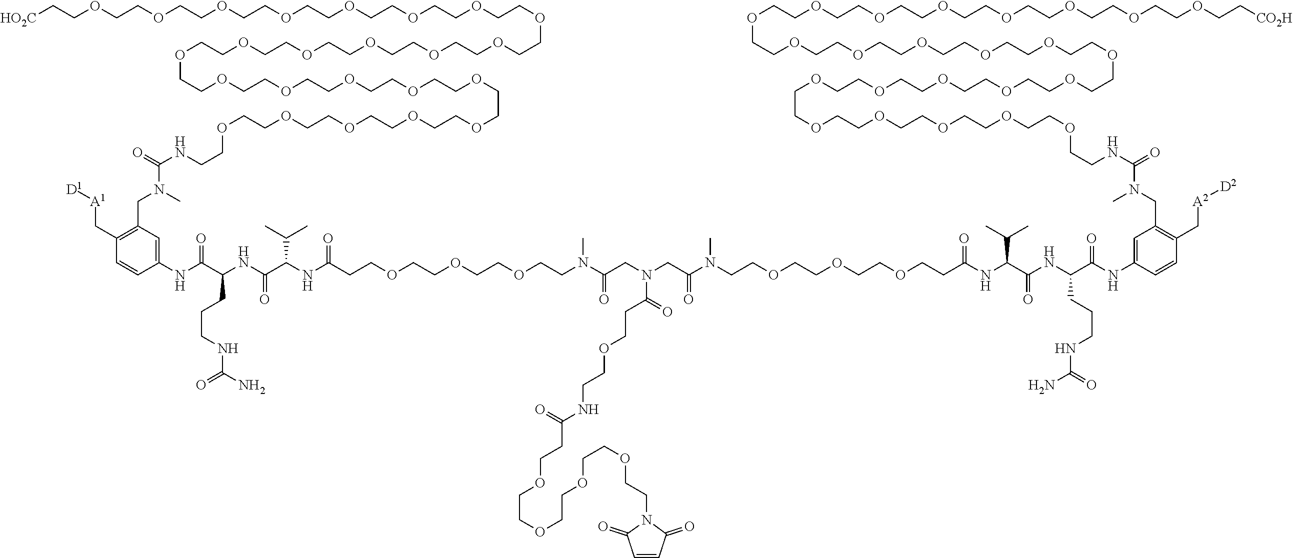

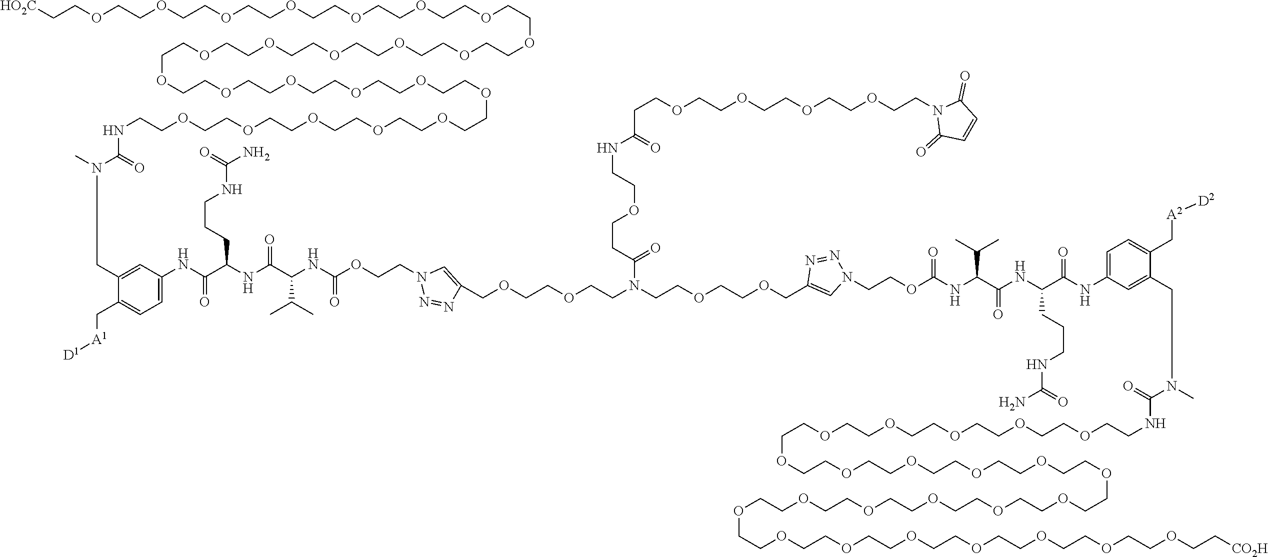

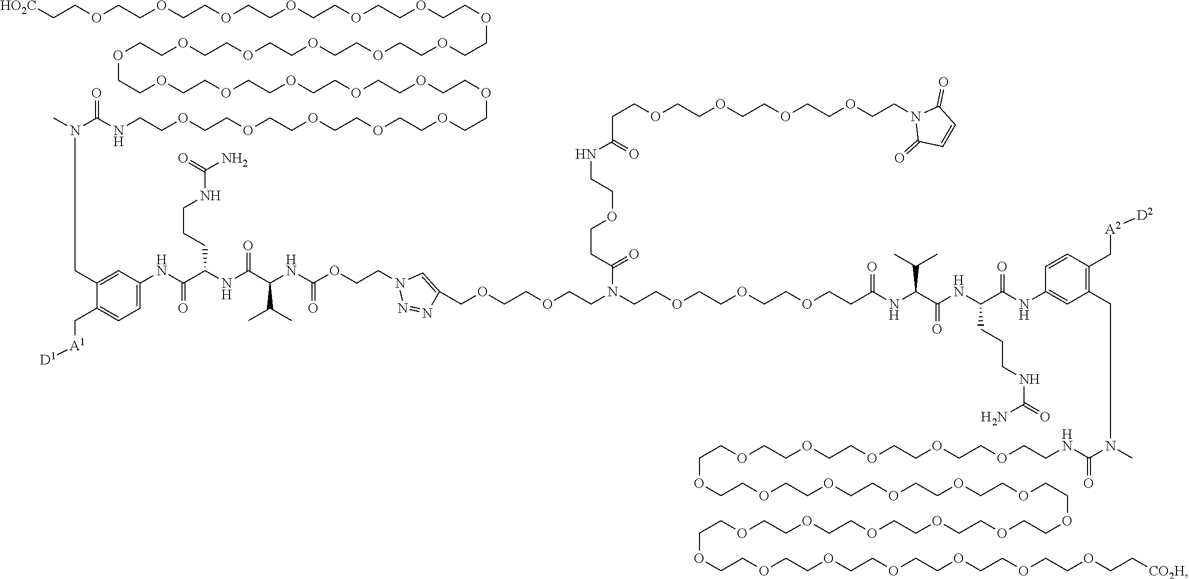

or a pharmaceutically acceptable salt thereof.
Embodiment 45e. The compound or pharmaceutically acceptable salt thereof according to any one of Embodiments 1 to 43, selected from:

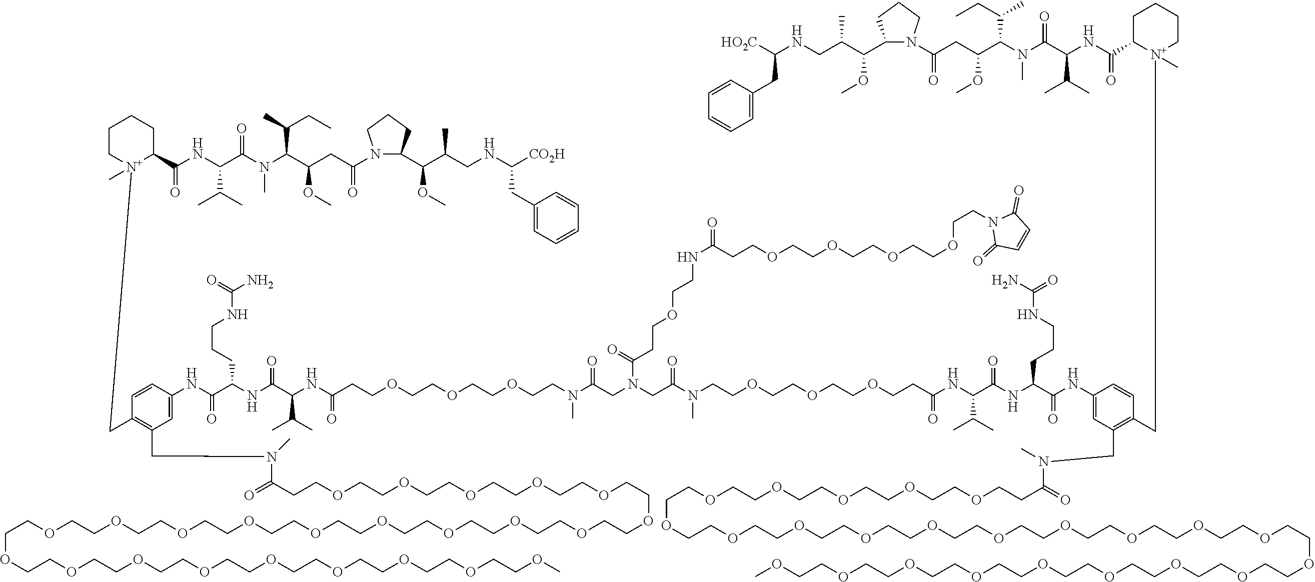

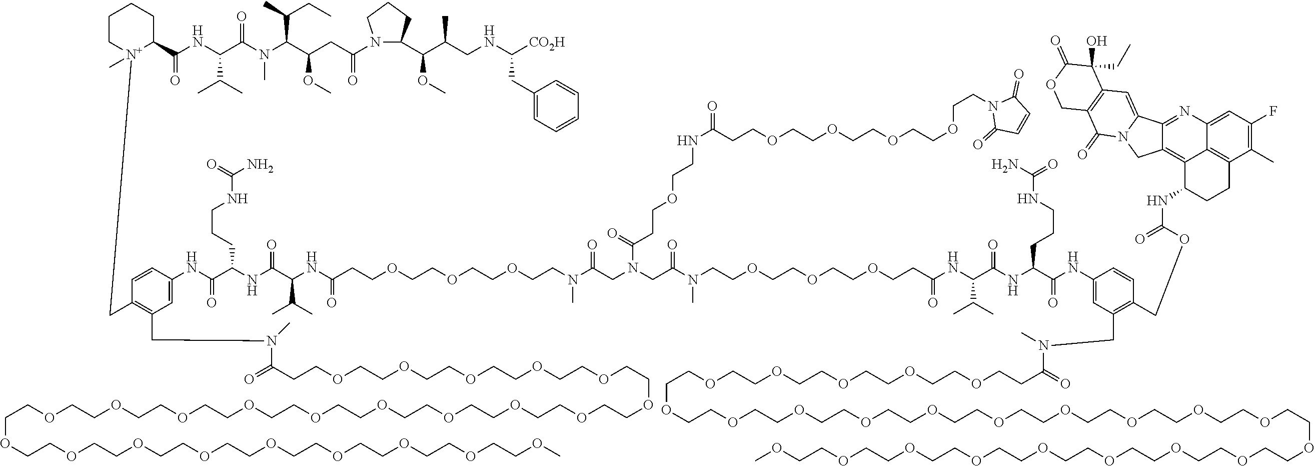

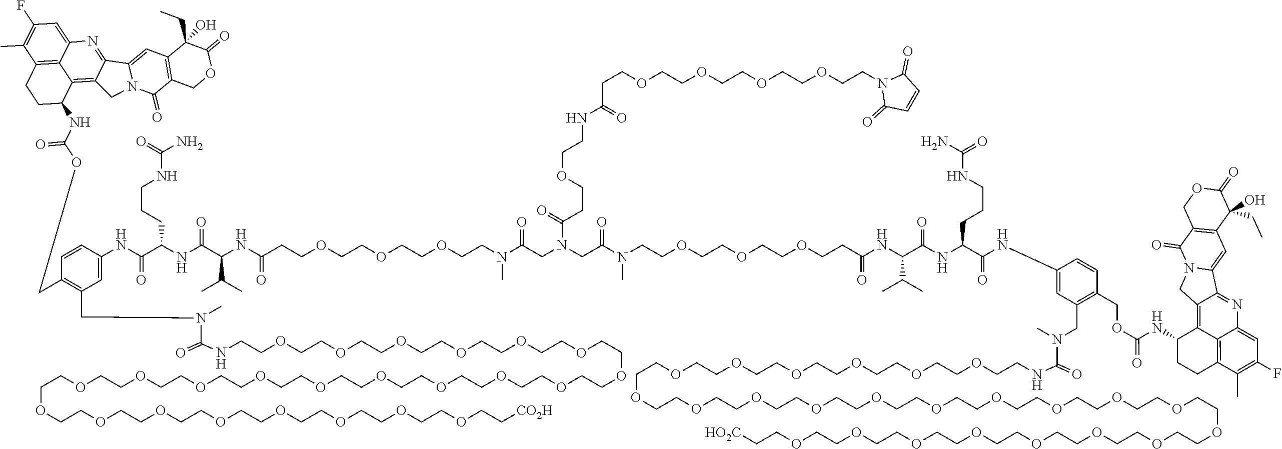

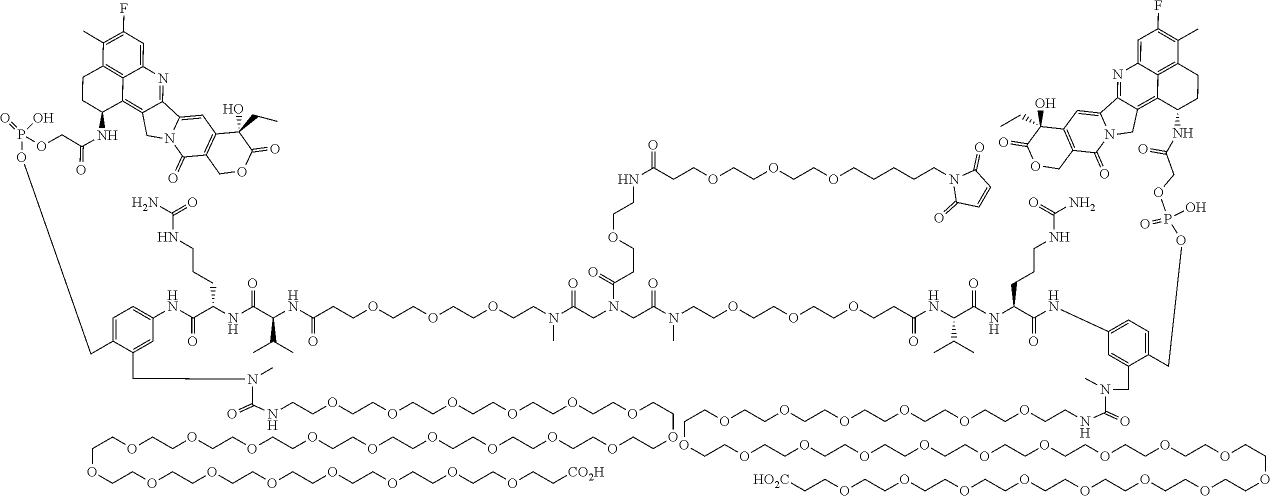

or a pharmaceutically acceptable salt thereof.
Embodiment 45f. The compound or pharmaceutically acceptable salt thereof according to any one of Embodiments 1 to 43, selected from:

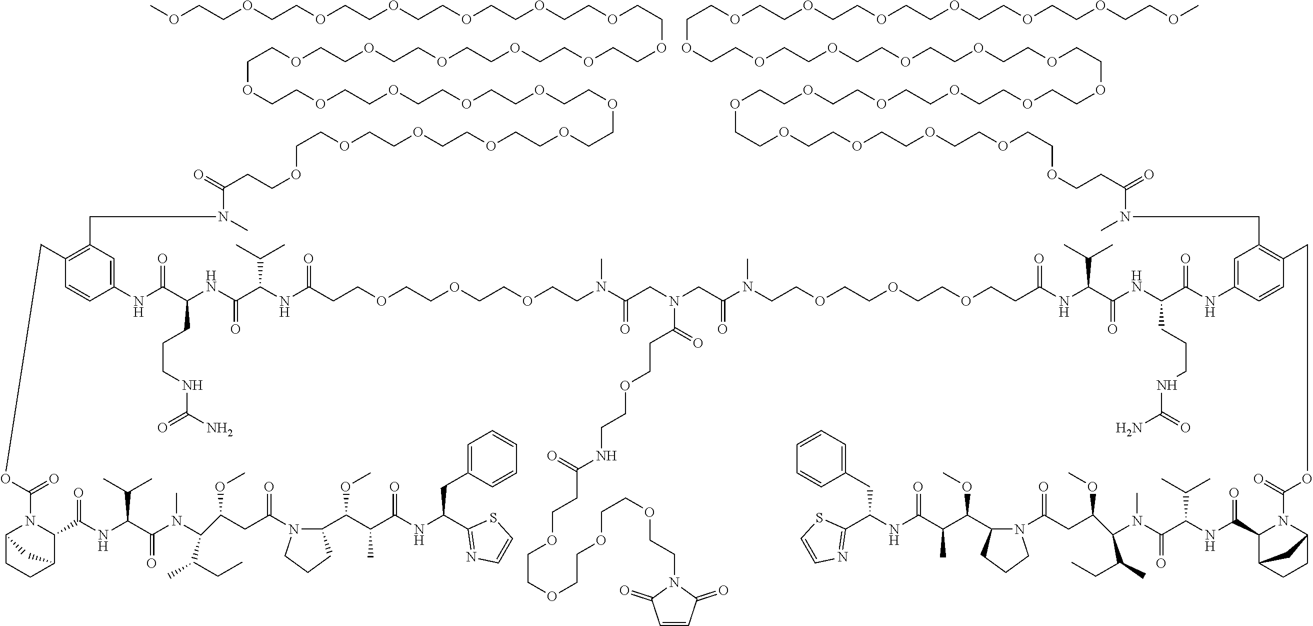

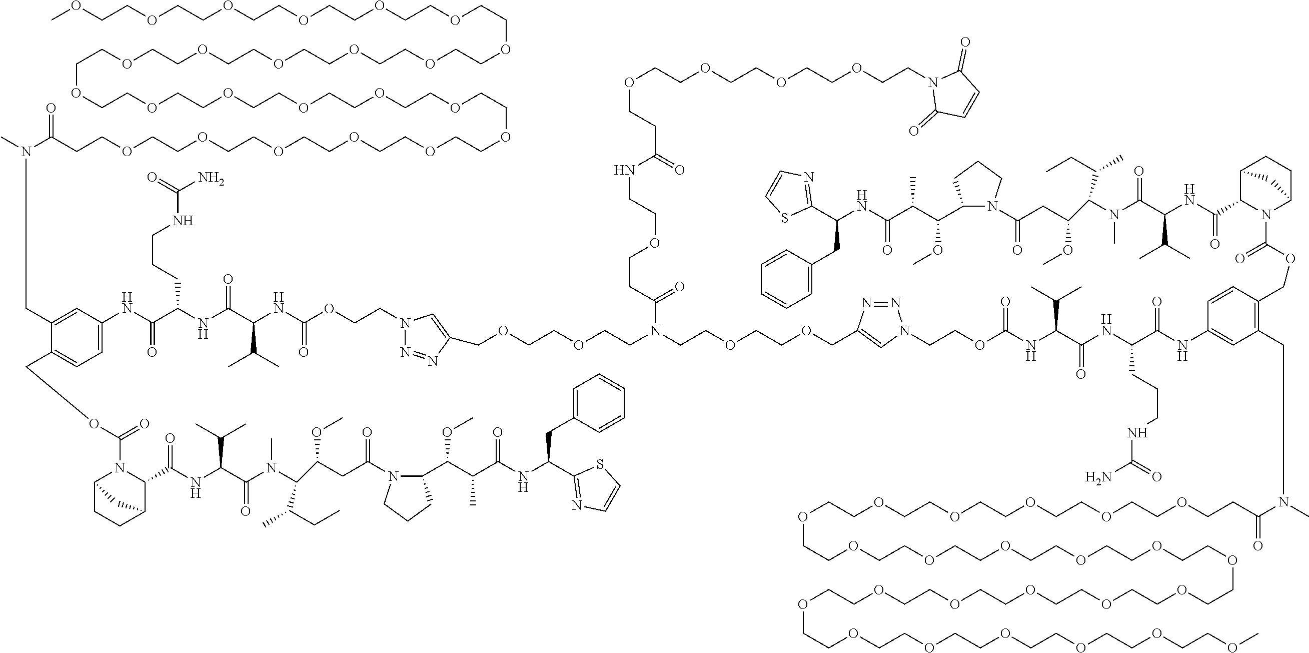

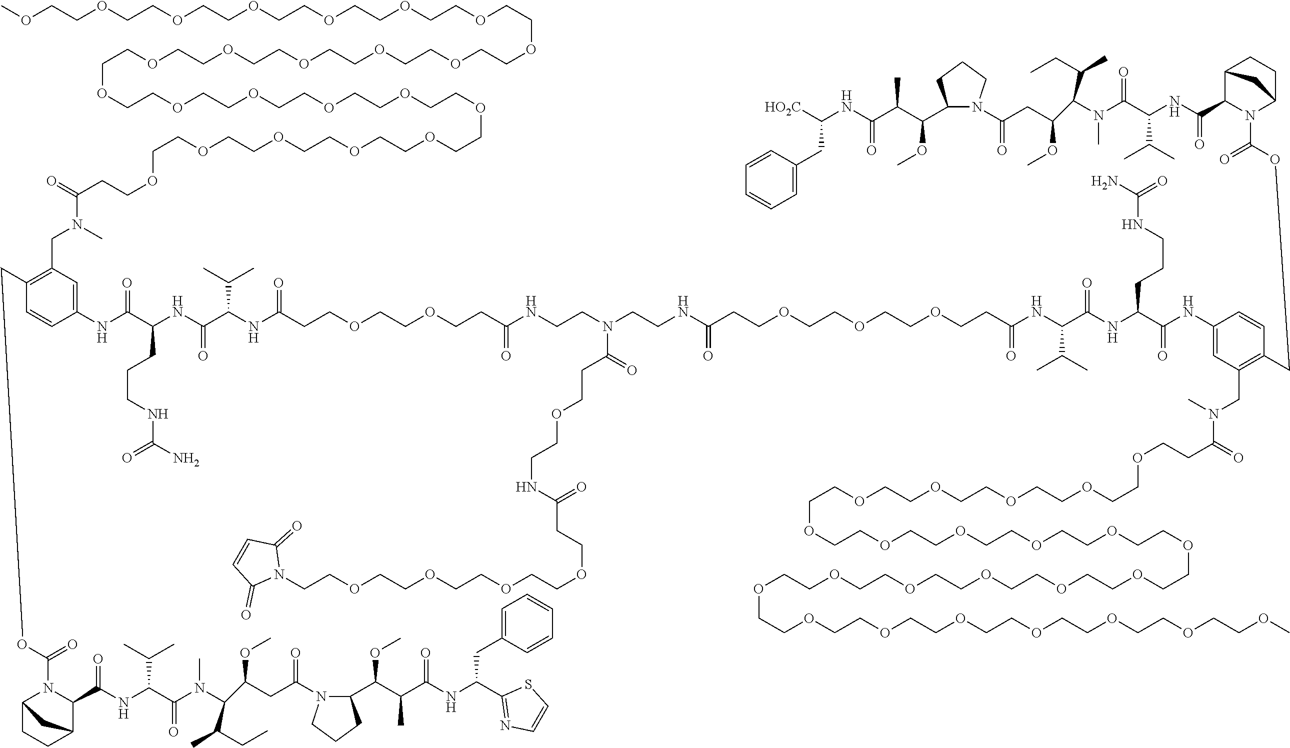

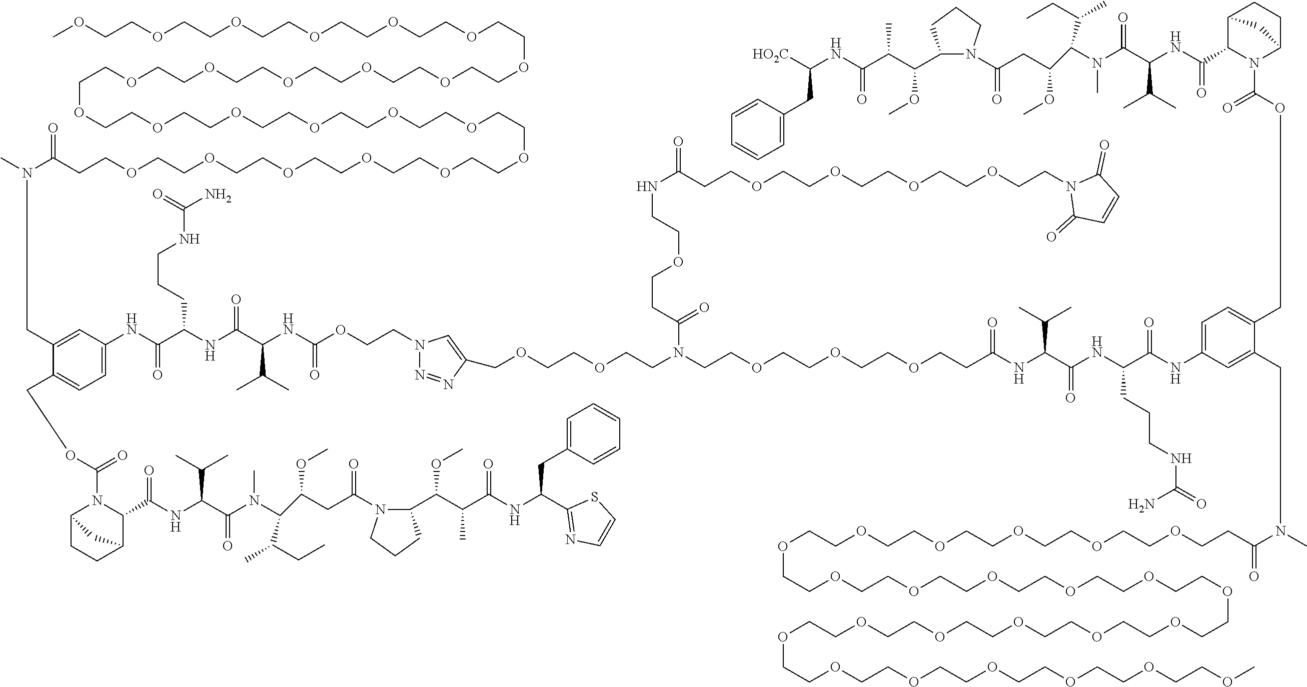

or a pharmaceutically acceptable salt thereof.
Embodiment 46. A conjugate comprising an antibody or an antigen-binding fragment thereof covalently linked to two pharmaceutically active drugs through a dual linker, and wherein the dual linker has one attachment point connected to the antibody and two attachment points to the two pharmaceutically active drugs, with the proviso that neither pharmaceutically active drug is a BH3 mimetic.
Embodiment 46a. The conjugate according to Embodiment 46, wherein the two pharmaceutically active drugs are the same or different.
Embodiment 46b. The conjugate according to Embodiment 46 or 46a, wherein the pharmaceutically active drug is selected from a cytotoxic drug, a cytostatic drug and an immunosuppressive drug.
Embodiment 46c. The conjugate according to any one of Embodiments 46 to 46b, wherein the pharmaceutically active drug is selected from an auristatin, a camptothecin, a duocarmycin, an etoposide, a maytansine, a maytansinoid, a taxane, a benzodiazepine or benzodiazepine containing drug (e.g., pyrrolo[1,4]-benzodiazepines (PBDs), indolinobenzodiazepines, and oxazolidinobenzodiazepines), and a vinca alkaloid.
Embodiment 46d. The conjugate according to Embodiment 46c, wherein the pharmaceutically active drug is an auristatin.
Embodiment 46e. The conjugate according to Embodiment 46 or 46a, wherein the pharmaceutically active drug is selected from an antitubulin agent, a tubulin inhibitor, a DNA minor groove binder, a DNA replication inhibitor, an alkylating agent, an antibiotic, an antifolate, an antimetabolite, a chemotherapy sensitizer, a topoisomerase inhibitor, and/or a vinca alkaloid.
Embodiment 46f. The conjugate according to Embodiment 46 or 46a, wherein the pharmaceutically active drug is selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor, a DHFR inhibitor, or a pro-apoptotic agent.
Embodiment 46 g. The conjugate according to Embodiment 46 or 46a, wherein the pharmaceutically active drug is selected from a topoisomerase 1 inhibitor, or an enantiomer, diastereoisomer, atropisomer, deuterated derivative, and/or addition salt thereof with a pharmaceutically acceptable acid or base.
Embodiment 46 h. The conjugate according to Embodiment 46 g, wherein the pharmaceutically active drug is wherein the topoisomerase 1 inhibitor is selected from topotecan, exatecan, deruxtecan and SN-38.
Embodiment 46i. The conjugate according to Embodiment 46 or 46a, wherein the pharmaceutically active drug is selected from an anti-mitotic drug, or an enantiomer, diastereoisomer, atropisomer, deuterated derivative, and/or addition salt thereof with a pharmaceutically acceptable acid or base.
Embodiment 46j. The conjugate according to Embodiment 46i, wherein the anti-mitotic drug is monomethyl auristatin E (MMAE) or a taxane.
Embodiment 46k. The conjugate according to Embodiment 46j, wherein the taxane is selected from docetaxel, paclitaxel, or cabazitaxel.
Embodiment 461. The conjugate according to Embodiment 46, wherein the conjugate comprises the compound or pharmaceutically acceptable salt thereof according to any one of Embodiments 1 to 45f.
Embodiment 46m. A conjugate of formula (A-2):

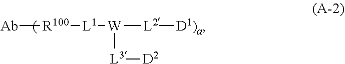

wherein:
Ab is an antibody or fragment thereof;
R¹⁰⁰ is a coupling group;
L¹ is a bridging spacer;
W is a branching moiety;
L^{2′} and L^{3′} are each independently a linker;
D¹ and D² are each independently a pharmaceutically active drug, with the proviso that neither D¹ nor D² is a BH3 mimetic; and
a is an integer from 1 to 16.
Embodiment 47. The conjugate according to Embodiment 46m, wherein each of L^{2′} and L^{3′} comprises a cleavable group, optionally at least one cleavable group comprises a phosphate group, a pyrophosphate group and/or a self-immolative group (e.g. a peptide group and a self-immolative group).
Embodiment 47a. The conjugate according to Embodiment 47, wherein each of L^{2′} and L^{3′} comprises a peptide group and a self-immolative group.
Embodiment 47b. The conjugate according to Embodiment 46m, wherein each of L^{2′} and L^{3′} comprises a cleavable group, optionally at least one cleavable group comprises a phosphate group, a pyrophosphate group, a sugar (e.g. glucuronamide) group, a peptide group, and/or a self-immolative group (e.g. a peptide group and a self-immolative group).
Embodiment 48. The conjugate according to Embodiment 46m, Embodiment 47 or Embodiment 47a, of formula (B-2):

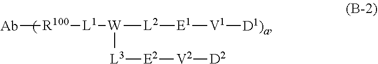

wherein:
Ab is an antibody or fragment thereof;
R¹⁰⁰ is a coupling group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently, an enzyme cleavage element or a hydrophilic moiety (e.g. E¹ and E² are each an enzyme cleavage element);
V¹ and V² independently comprise i) a self-immolative group, ii) an enzyme cleavage element (e.g. V¹ and V² are each independently a self-immolative group), or iii) a self-immolative group and an enzyme cleavage element;
D¹ and D² are each independently a pharmaceutically active drug, with the proviso that neither D¹ nor D² is a BH3 mimetic; and
a is an integer from 1 to 16.
Embodiment 48aa. The conjugate according to Embodiment 48, wherein V¹ and V² are each independently i) a self-immolative group, or ii) an enzyme cleavage element (e.g. V¹ and V² are each independently a self-immolative group).
Embodiment 48a. The conjugate according to Embodiment 48 or Embodiment 48aa, wherein W is N or CH.
Embodiment 48b. The conjugate according to Embodiment 48, Embodiment 48aa or Embodiment 48a, wherein E¹ and E² are each an enzyme cleavage element.
Embodiment 48c. The conjugate according to Embodiment 48, Embodiment 48aa, Embodiment 48a or Embodiment 48b, wherein V¹ and V² are each independently a self-immolative group.
Embodiment 49. The conjugate according to any one of Embodiments 46m to 48c, wherein a is an integer from 1 to 8, 1 to 6, 1 to 4, or a is 1 or 2.
Embodiment 49a. The conjugate according to Embodiments 46m to 49, wherein a is determined by liquid chromatography-mass spectrometry (LC-MS).
Embodiment 50. The conjugate according to any one of Embodiments 46m to 49, wherein (i) the cleavable linker comprises a phosphate group, a pyrophosphate group and/or a self-immolative group; (ii) the cleavable linker comprises a self-immolative group; or (iii) the cleavable linker comprises a self-immolative group comprising —CH₂—O—, —NH—CH₂—, —C(═O)—, para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.
Embodiment 50a. The conjugate according to Embodiment 50, wherein W is N or CH.
Embodiment 50b. The conjugate according to Embodiment 50 or Embodiment 50a, wherein R² and R³ are each a hydrophilic group.
Embodiment 51. The conjugate according to any one of Embodiments 46m to 50b, of formula (C-2):

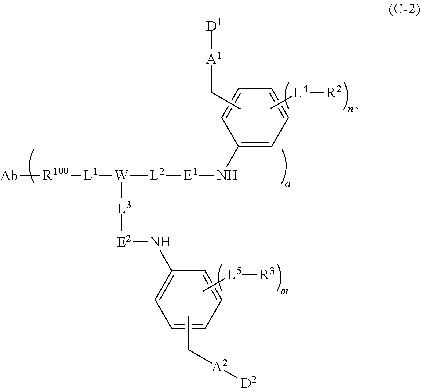

wherein:
Ab is an antibody or antigen-binding fragment thereof;
R¹⁰⁰ is a coupling group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently a peptide group comprising 1 to 6 amino acids, wherein said peptide group is optionally substituted by a hydrophilic group;

A¹ and A² are each independently a bond, —OC(═O)—*,

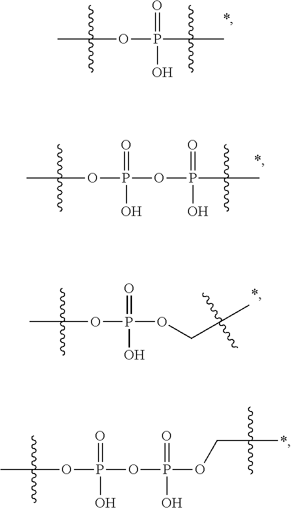

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆alkyl, and C₃-C₈cycloalkyl and the * of A¹ or A² indicates the point of attachment to D¹ or D²;
D¹ and D² are each independently a pharmaceutically active drug, with the proviso that neither D¹ nor D² is a BH3 mimetic;
L⁴ and L⁵ are each independently a spacer moiety;
R² and R³ are each independently a hydrophilic group or an enzyme cleavage element (e.g. R² and R³ are each a hydrophilic group); and
m and n are each independently 0 or 1.
Embodiment 52. The conjugate according to Embodiment 51, of formula (D1-2), (D2-2) or (D3-2):

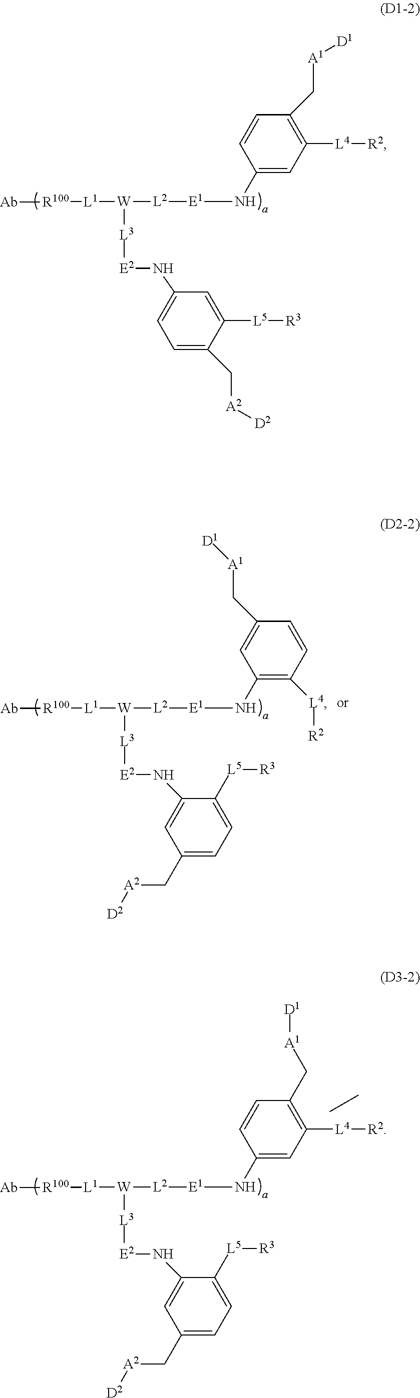

Embodiment 53. The conjugate according to Embodiment 52, wherein for Formula (D1-2), R² and R³ are each independently a hydrophilic group, wherein for Formula (D2-2), R² and R³ are each independently an enzyme cleavage element; and for Formula (D3-2), R² is a hydrophilic group and R³ is an enzyme cleavage element.
Embodiment 54. The conjugate according to any one of Embodiments 46m to 53, wherein R¹⁰⁰ is selected from the group consisting of

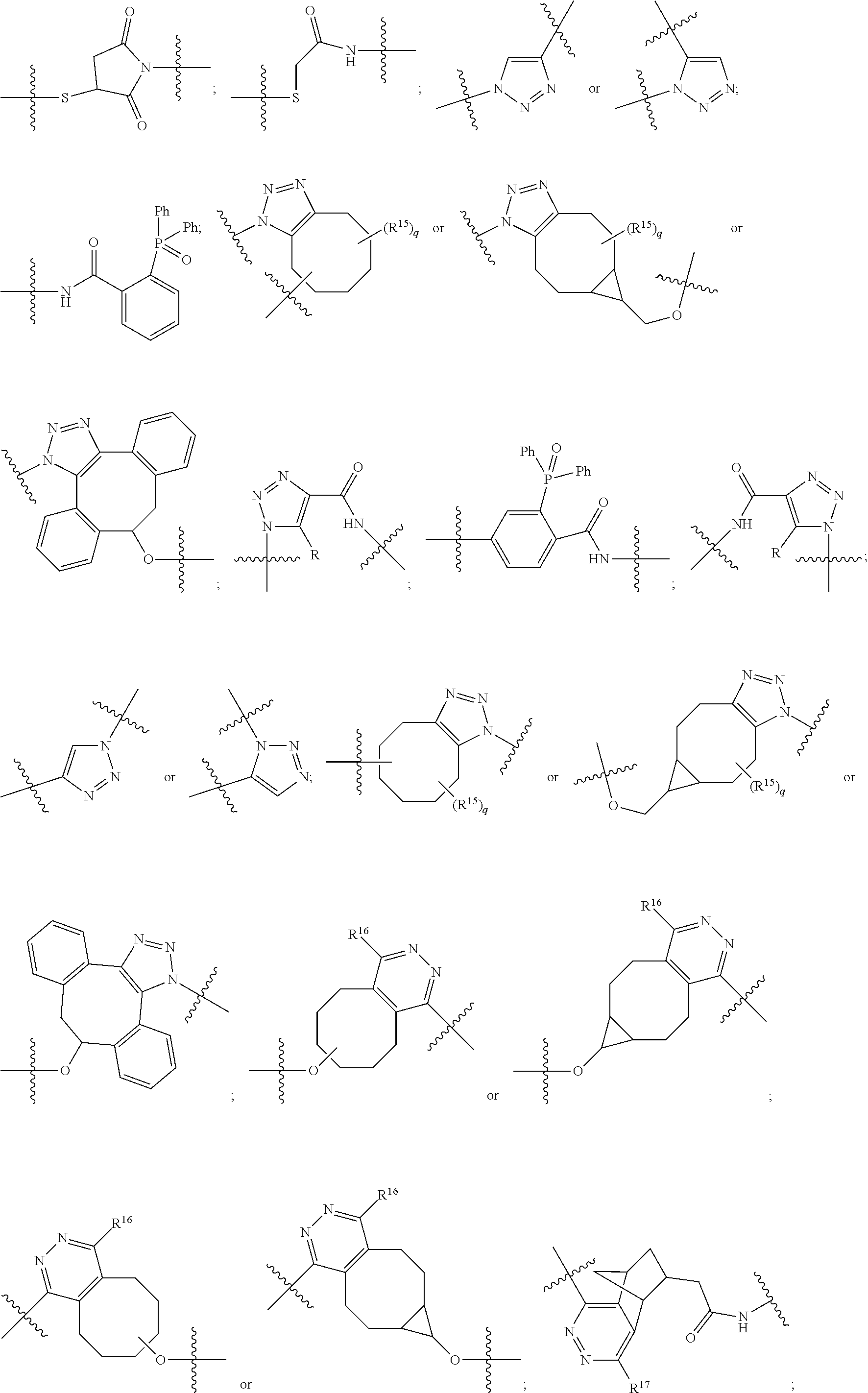

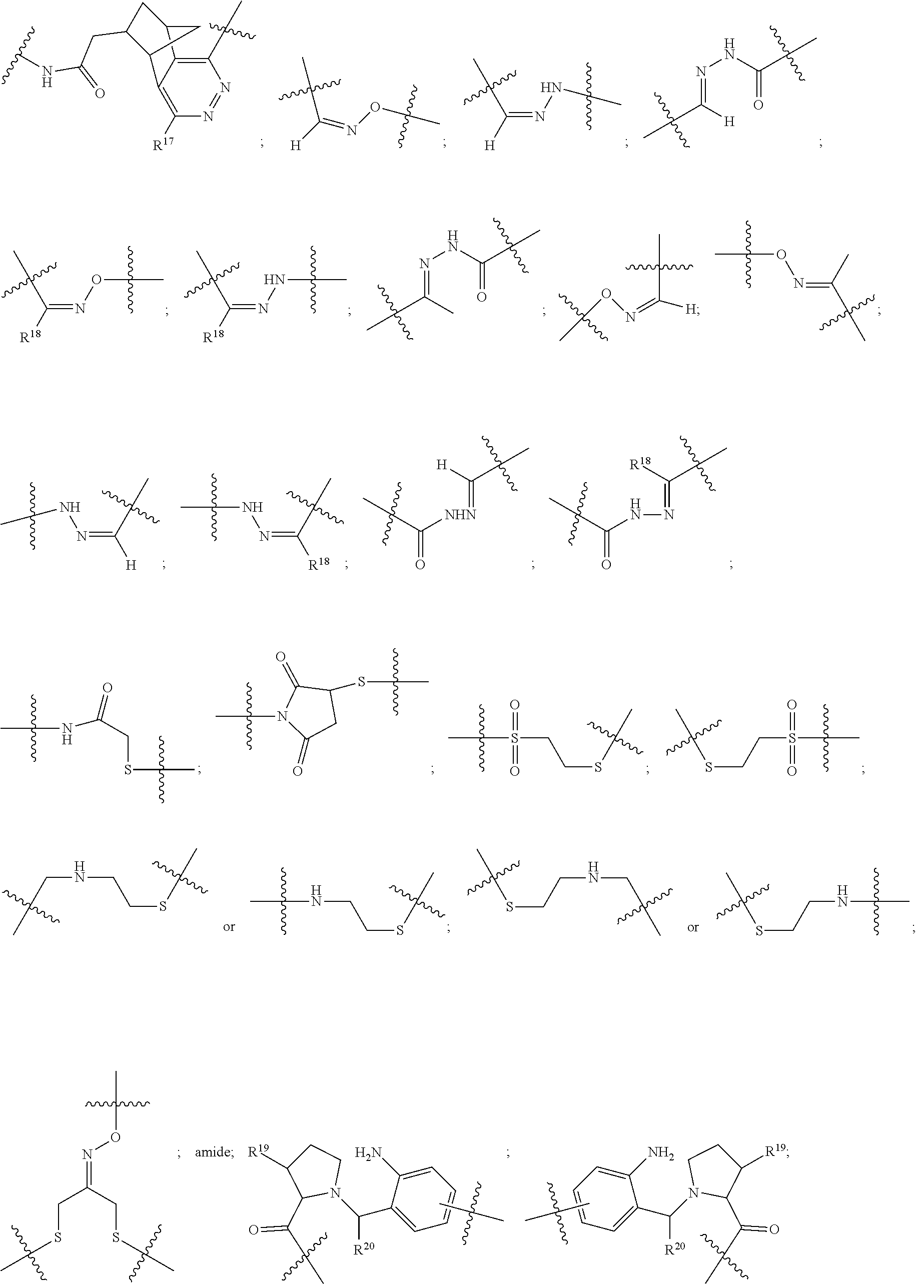

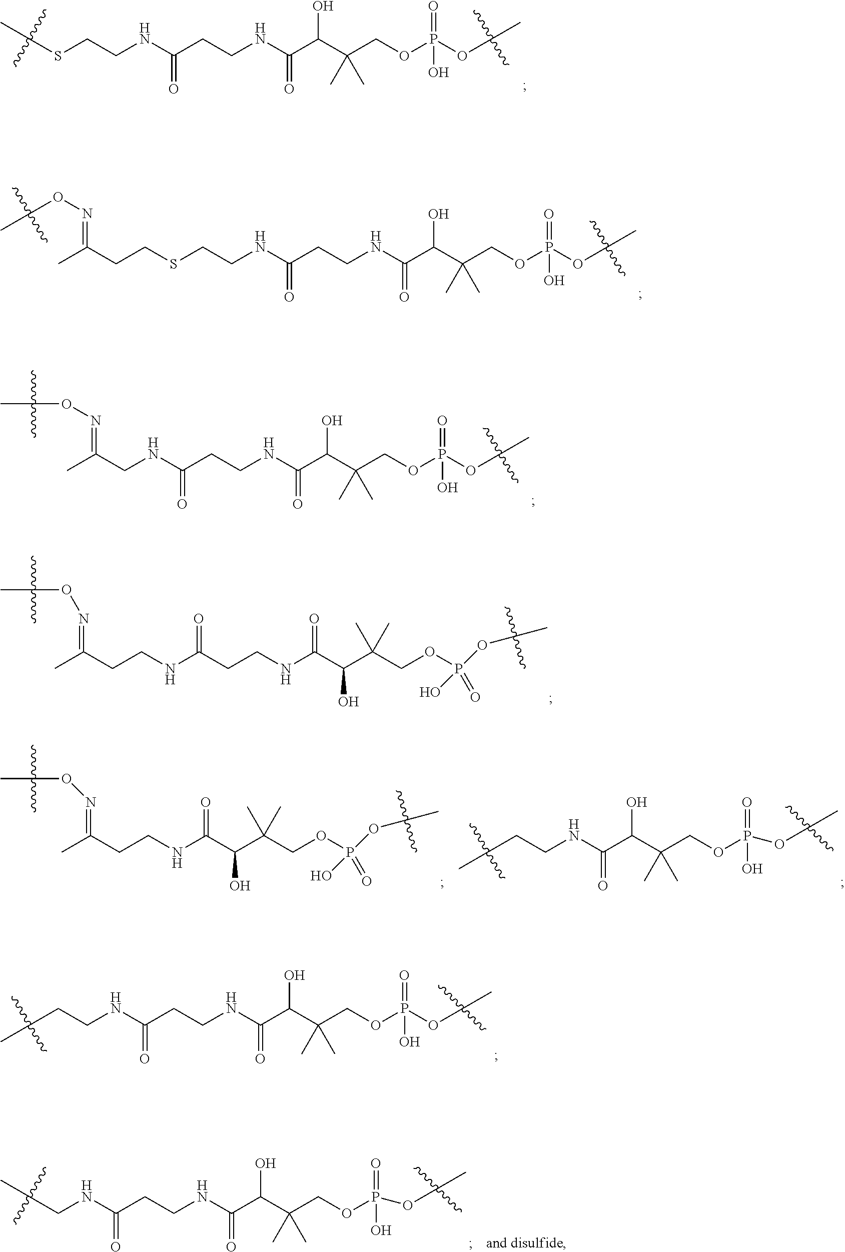

wherein:
   R¹⁶ is H, C₁-C₄alkyl, phenyl, pyrimidine or pyridine;
   R¹⁸ is H, C₁-C₆alkyl, phenyl or C₁-C₄alkyl substituted with 1 to 3 —OH groups;
   each R¹⁵ is independently selected from H, C₁-C₆alkyl, fluoro, benzyloxy substituted with —C(═O)OH, benzyl substituted with —C(═O)OH, C₁-C₄alkoxy substituted with —C(═O)OH and C₁-C₄alkyl substituted with —C(═O)OH;
   R¹⁷ is independently selected from H, phenyl and pyridine;
   q is 0, 1, 2 or 3;
   R¹⁹ is H or methyl; and
   R²⁰ is H, —CH₃ or phenyl.
Embodiment 55. The conjugate according to any one of Embodiments 46m to 54, wherein R¹⁰⁰ is selected from the group consisting of

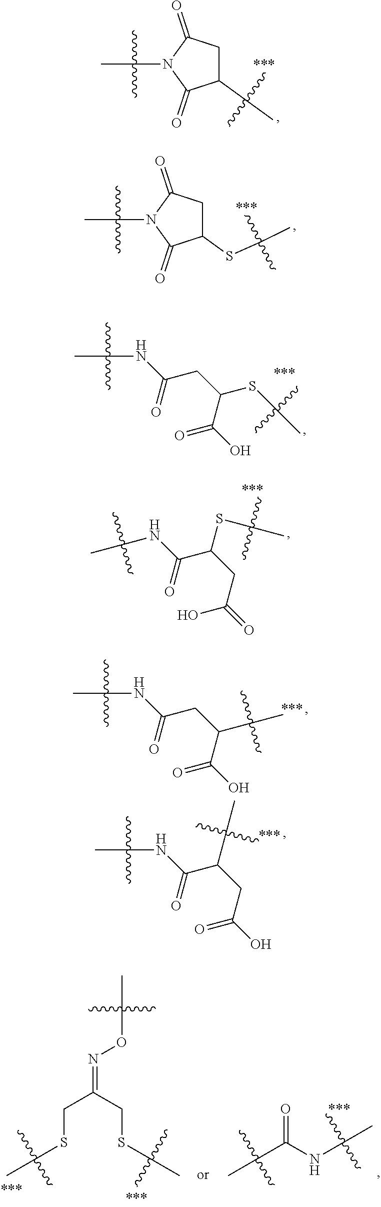

where the *** of R¹⁰⁰ indicates the point of attachment to Ab.
Embodiment 56. The conjugate according to Embodiment 55, wherein R¹⁰⁰ is

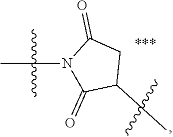 [SMILES: *C1CC(=O)N(*)C1=O]

where the *** of R¹⁰⁰ indicates the point of attachment to Ab.
Embodiment 57. The conjugate according to any one of the Embodiments 46m to 56, wherein:
   (1) L¹ comprises:

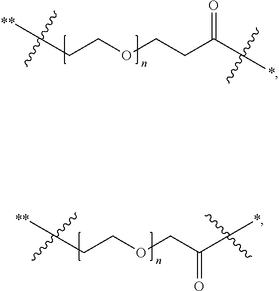

or *—CH(OH)CH(OH)CH(OH)CH(OH)—**,
wherein each n is an integer from 1 to 12, wherein the * of L¹ indicates the point of direct or indirect (e.g. direct) attachment to W, and the ** of L¹ indicates the point of direct or indirect (e.g. direct) attachment to R¹;
(2) L¹ is

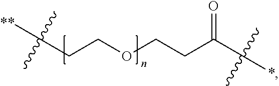

and n is an integer from 1 to 12 or n is 1 or n is 12, wherein the * of L¹ indicates the point of direct or indirect (e.g. direct) attachment to W, and the ** of L¹ indicates the point of direct or indirect (e.g. direct) attachment to R¹;
(3) L¹ is

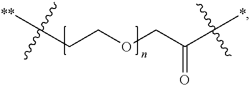

and n is an integer from 1 to 12, wherein the * of L¹ indicates the point of direct or indirect (e.g. direct) attachment to W, and the ** of L₁ indicates the point of direct or indirect (e.g. direct) attachment to R¹;
(4) L¹ comprises

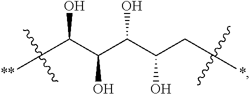

wherein the * of L¹ indicates the point of direct or indirect (e.g. direct) attachment to W, and the ** of L¹ indicates the point of direct or indirect (e.g. direct) attachment to R¹; or
   (5) L¹ is a bridging spacer comprising:
*—C(═O)(CH₂)ₘO(CH₂)ₘ—**; *—C(═O)((CH₂)ₘO)ₜ(CH₂)ₙ—**; *—C(═O)(CH₂)ₘ—**;
*—C(═O)NH((CH₂)ₘO)ₜ(CH₂)ₙ—**;
*—C(═O)O(CH₂)ₘSSC(R^{L1})₂(CH₂)ₘC(═O)NR^{L1} (CH₂)ₘNR^{L1}C(═O)(CH₂)ₘ—**;
*—C(═O)O(CH₂)ₘC(═O)NH(CH₂)ₘ—**; *—C(═O)(CH₂)ₘNH(CH₂)ₘ—**;
*—C(═O)(CH₂)ₘNH(CH₂)ₙC(═O)—**; *—C(═O)(CH₂)ₘX¹(CH₂)ₘ—**;
*—C(═O)((CH₂)ₘO)ₜ(CH₂)ₙX₁(CH₂)ₙ—**; *—C(═O)(CH₂)ₘNHC(═O)(CH₂)ₙ—**;
*—C(═O)((CH₂)ₘO)ₜ(CH₂)ₙNHC(═O)(CH₂)ₙ—**;
*—C(═O)(CH₂)ₘNHC(═O)(CH₂)ₙX₁(CH₂)ₙ—**;
*—C(═O)((CH₂)ₘO)ₜ(CH₂)ₙNHC(═O)(CH₂)ₙX₁(CH₂)ₙ—**;
*—C(═O)((CH₂)ₘO)ₜ(CH₂)ₙC(═O)NH(CH₂)ₘ—**; *—C(═O)(CH₂)ₘC(R^{L1})₂—** or
*—C(═O)(CH₂)ₘC(═O)NH(CH₂)ₘ—**, wherein the * of L¹ indicates the point of direct or indirect (e.g. direct) attachment to W, and the ** of L¹ indicates the point of direct or indirect (e.g. direct) attachment to R¹;
X₁ is

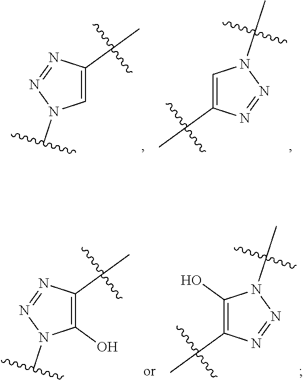 [SMILES: *c1cn(*)nn1 ; *c1cn(*)nn1 ; *c1nnn(*)c1O ; *c1nnn(*)c1O]

and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30; and
each R^{L1} is independently selected from H and C₁-C₆alkyl.
Embodiment 58. The conjugate according to any one of Embodiments 46m to 57, wherein L¹ comprises a moiety represented by

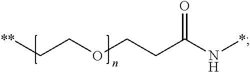

wherein n is an integer from 1 to 12 (e.g. 4 to 8, e.g. 4 or 8), wherein the * of L¹ indicates the point of direct or indirect (e.g. direct) attachment to W, and the ** of L¹ indicates the point of direct or indirect attachment to R¹.
Embodiment 58a. The conjugate according to Embodiment 58, wherein n is an integer from 4 to 8.
Embodiment 58b. The conjugate according to Embodiment 58a, wherein n is 4 or 8.
Embodiment 59. The conjugate according to any one of Embodiments 46m to 58b, wherein L¹ is represented by a formula

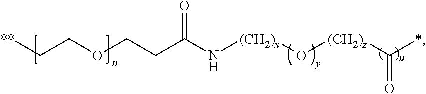

wherein
   n is an integer from 1 to 12 (e.g. 4 to 8, e.g. 4 or 8);
   x is an integer from 0 to 6 (e.g. 0 to 4, e.g. 0 to 2, e.g. 0 or 2);
   y is 0 or 1;
   z is an integer from 0 to 6 (e.g. 0 to 4, e.g. 0 to 2, e.g. 0 or 2);
   u is 0 or 1; and
   wherein the * of L¹ indicates the point of direct attachment to W, and the ** of L¹ indicates the point of direct attachment to R¹.
Embodiment 59a. The conjugate according to Embodiment 59, wherein n is an integer from 4 to 8.
Embodiment 59b. The conjugate according to Embodiment 59a, wherein n is 4 or 8.
Embodiment 59c. The conjugate according to Embodiment 59, Embodiment 59a or Embodiment 59b, wherein x is an integer from 0 to 2.
Embodiment 59d. The conjugate according to Embodiment 59c, wherein x is 0 or 2.
Embodiment 59e. The conjugate according to Embodiment 59, Embodiment 59a,
Embodiment 59b, Embodiment 59c or Embodiment 59d, wherein z is an integer from 0 to 2.
Embodiment 59f. The conjugate according to Embodiment 59e, wherein z is 0 or 2.
Embodiment 60. The conjugate according to any one of Embodiments 46m to 59f, wherein L¹ is selected from the group consisting of:

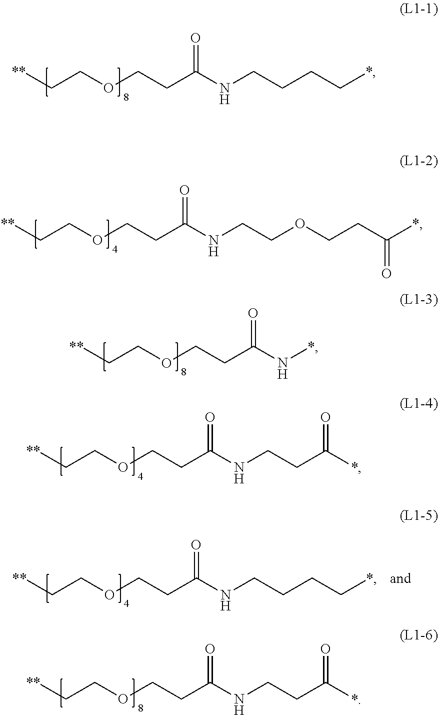

Embodiment 61. The conjugate according to any one of Embodiments 48 to 60, wherein L² and L³ are each independently a connecting spacer comprising a moiety represented by:

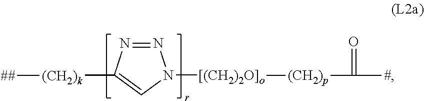

wherein
   k is an integer from 0 to 6;
   r is 0 or 1;
   is an integer from 0 to 12;
   p is an integer from 0 to 6; and
wherein the # of L² or L³ indicates the point of direct or indirect attachment to E¹ or E², respectively, and the ## of L² or L³ indicates the point of direct or indirect attachment to W.
Embodiment 62. The conjugate according to any one of Embodiments 48 to 60, wherein L² and L³ are each independently a connecting spacer selected from a group consisting of:

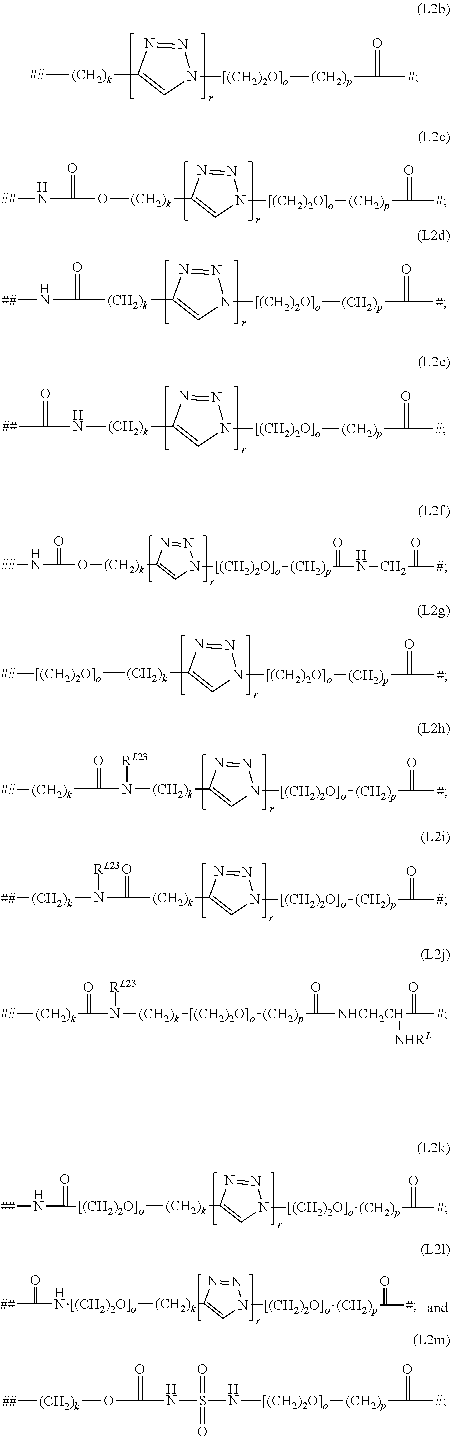

wherein
   k, in each occurrence, is independently an integer from 0 to 4;
   r, in each occurrence, is independently 0 or 1;
   o, in each occurrence, is independently an integer from 0 to 10;
   p, in each occurrence, is independently an integer from 0 to 4;
   R^{L23} is hydrogen or C₁-C₆alkyl;
   R^{L} is hydrogen or —C(O)—R^{H};
   R^{H} is a hydrophilic group; and
   the # of L² or L³ indicates the point of direct attachment to E¹ or E², respectively, and the ## of L² or L³ indicates the point of direct attachment to W;
   provided that when W is N, L² and L³ are not (L2c), (L2d), (L2f) or (L2k).
Embodiment 63. The conjugate according to Embodiment 62, wherein L² and L³ are each independently a connecting spacer selected from a group consisting of:

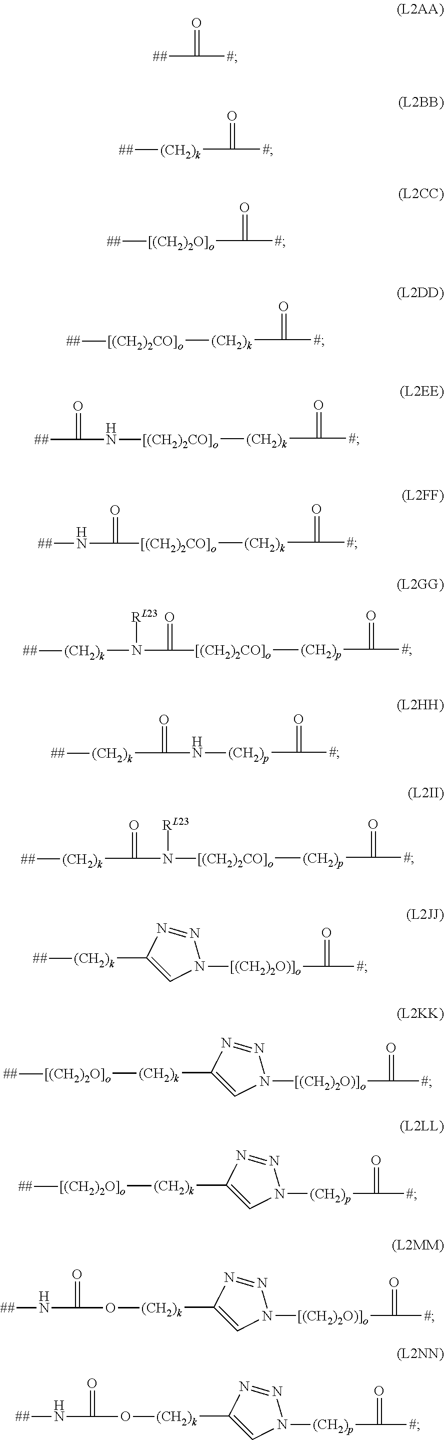

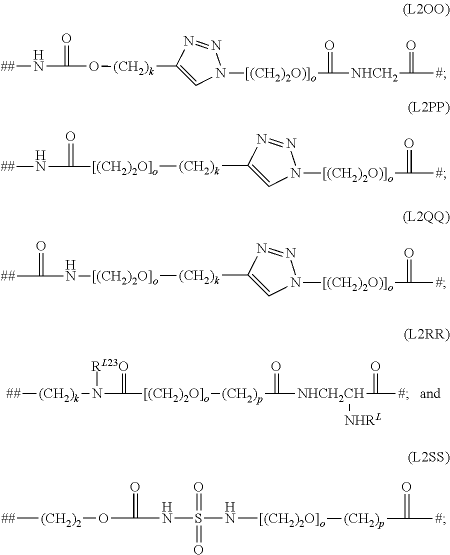

wherein
k, in each occurrence, is independently an integer from 1 to 3;
o, in each occurrence, is independently an integer from 1 to 9;
p, in each occurrence, is independently an integer from 1 to 3;
R^{L23} is hydrogen or C₁-C₃alky;
R^{L} is hydrogen or —C(O)—R^{H};
R^{H} is a hydrophilic group; and
the # of L² or L³ indicates the point of direct attachment to E¹ or E², respectively, and the ## of L² or L³ indicates the point of direct attachment to W; provided that when W is N, L² and L³ are not (L2FF), (L2MM), (L2NN), (L2OO), or (L2PP).
Embodiment 64. The conjugate according to any one of Embodiments 48 to 63, wherein L² and L³, independently, are a connecting spacer selected from a group consisting of:

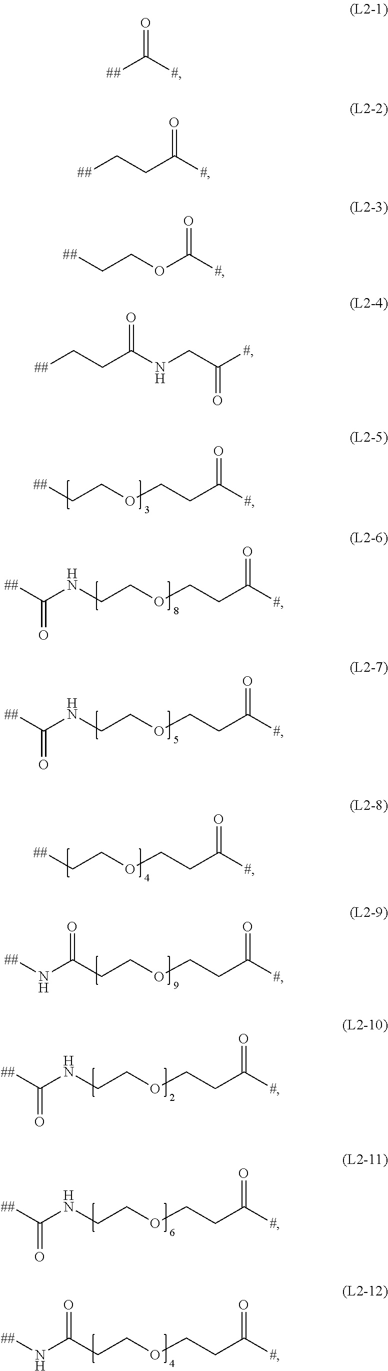

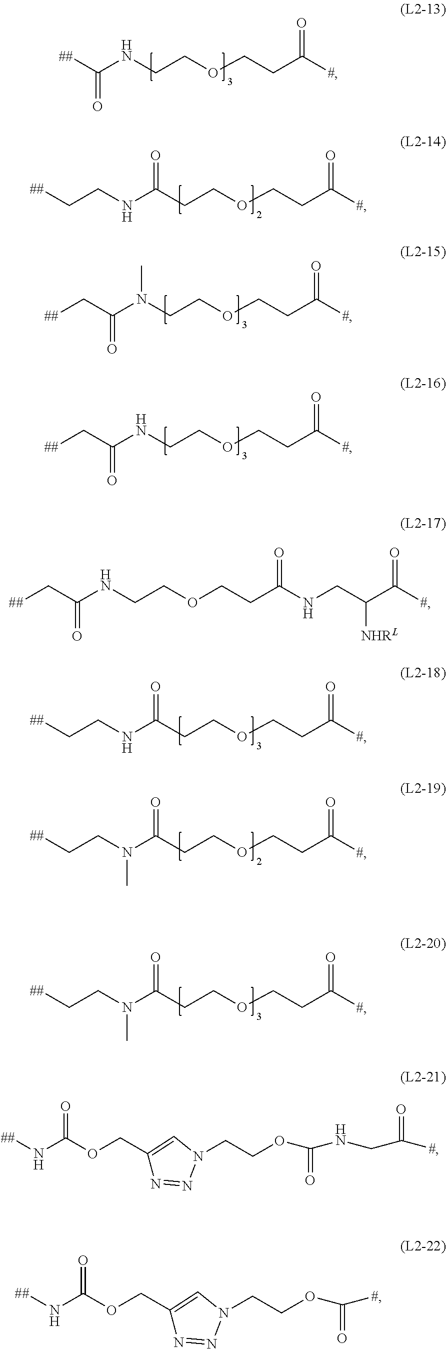

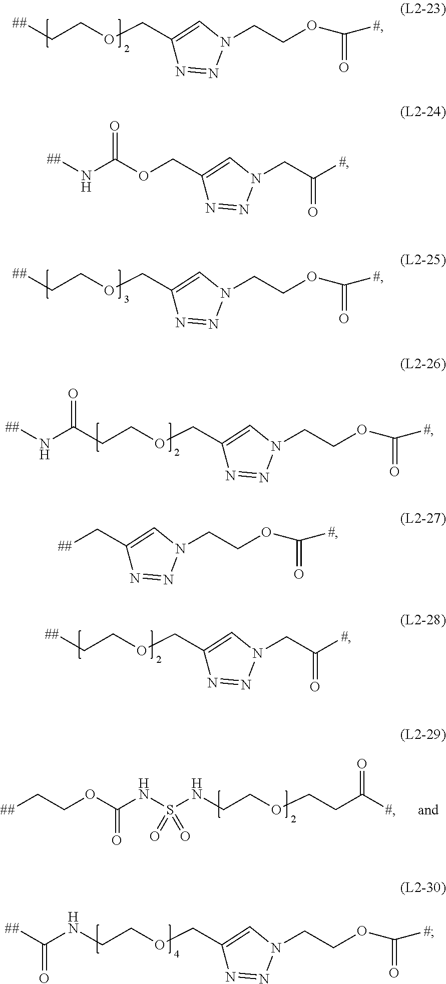

wherein the # of L² or L³ indicates the point of direct attachment to E¹ or E², respectively, the ## of L² or L³ indicates the point of direct attachment to W; R^{L} is hydrogen or —C(O)—R^{H}; and
R^{H} is

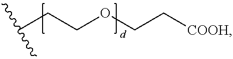

and d is an integer from 20 to 30 (e.g. 25).
Embodiment 64a. The conjugate according to Embodiment 64, wherein d is 25.
Embodiment 65. The conjugate according to any one of Embodiments 51 to 64a, wherein each peptide group independently comprises 1 to 4, 1 to 3, or 1 to 2 amino acid residues.
Embodiment 65a. The conjugate according to Embodiment 65, wherein each peptide group independently comprises 2 amino acid residues.
Embodiment 66. The conjugate according to Embodiment 65 or Embodiment 65a, wherein each amino acid residues is independently selected from glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), L-tyrosine (Tyr) and β-alanine (β-Ala).
Embodiment 67. The conjugate according to any one of Embodiments 51 to 65, wherein each peptide group is independently selected from Val-Cit, Phe-Lys, Val-Ala, Val-Lys, Leu-Cit, Cit-(R-Ala), Gly-Gly-Gly, Gly-Gly-Phe-Gly, and sulfo-Ala-Val-Ala.
Embodiment 68. The conjugate according to any one of Embodiments 48 to 65, wherein E¹ and/or E², independently, is/are each a peptide group selected from a group consisting of:

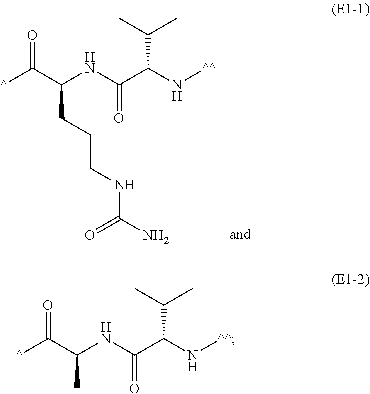

wherein \hat{ } of E1-1 or E1-2 indicates the point of direct attachment to V¹ or V² in Formula (B) or direct attachment to the —NH— group in Formula (C) and (D); and \hat{ }\hat{ } of E1-1 or E1-2 indicates the point of direct attachment to L² or L³, respectively (e.g. where E¹ and E² are each a peptide group independently selected from (E1-1) and (E1-2)).
Embodiment 68a. The compound or pharmaceutically acceptable salt thereof according to Embodiment 68, wherein E¹ and E² are each a peptide group independently selected from (E1-1) and (E1-2).
Embodiment 69. The conjugate according to any one of Embodiments 48 to 65, wherein E¹ and/or E², independently, is/are each a peptide group represented by

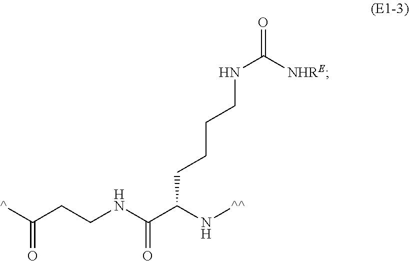

wherein R^{E} is a hydrophilic group R^{H}.
Embodiment 70. The conjugate according to Embodiment 69, wherein the hydrophilic group R^{H} in (E1-3) is

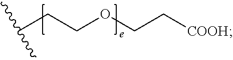

wherein e is an integer between 20 and 30 (e.g. 25).
Embodiment 70a. The conjugate according to Embodiment 70, wherein e is 25.
Embodiment 71. The conjugate according to Embodiment 68, wherein E¹ and E² are each

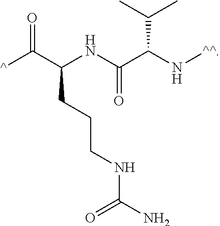

Embodiment 72. The conjugate according to any one of Embodiments 51 to 71, wherein A¹ and A² are independently selected from a bond and —OC(═O)—*, wherein * indicates the point of attachment to D¹ or D² respectively.
Embodiment 72a. The conjugate according to any one of Embodiments 51 to 71, wherein A¹ and A² are independently selected from a bond,

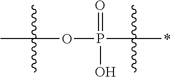

and —OC(═O)—*, wherein * indicates the point of attachment to D¹ or D² respectively.
Embodiment 73. The conjugate according to Embodiment 72, wherein A¹ and A² are a bond.
Embodiment 73a. The conjugate according to Embodiment 72, wherein A¹ and A² are both-OC(═O)—*.
Embodiment 73b. The conjugate according to Embodiment 72, wherein one of A¹ and A² is OC(═O)—*, and the other of A¹ and A² is a bond.
Embodiment 73c. The conjugate according to Embodiment 72a, wherein A¹ and A² are independently a bond or

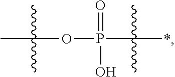

wherein * indicates the point of attachment to D¹ or D² respectively.
Embodiment 73d. The conjugate according to Embodiment 72a, wherein one of A¹ and A² is

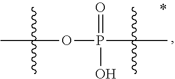 [SMILES: *OP(*)(=O)O]

and the other of A¹ and A² is a bond.
Embodiment 73e. The conjugate according to Embodiment 72a, wherein A¹ and A² are both

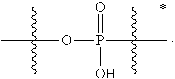 [SMILES: *OP(*)(=O)O]

Embodiment 73f. The conjugate according to Embodiment 72a, wherein one of A¹ and A² is

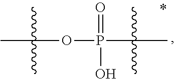 [SMILES: *OP(*)(=O)O]

and the other of A¹ and A² is OC(═O)—*.
Embodiment 73 g. The conjugate according to Embodiment 72a, wherein) A¹ and A² a-e —OC(═O)—*;
(ii) A¹ and A² are

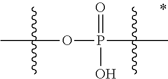 [SMILES: *OP(*)(=O)O]

(iii) A¹-s —OC(═O)—* and A² is a bond
(iv) A¹-s —OC(═O)—* and A² is

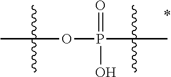 [SMILES: *OP(*)(=O)O]

(v) A¹ is a bond and A² is

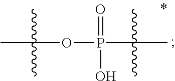 [SMILES: *OP(*)(=O)O]

or
(vi) A¹ is a bond and A²-s —OC(═O)—*,
wherein * indicates the point of attachment to D¹ or D².
Embodiment 74. The conjugate according to any one of Embodiments 51 to 73b, wherein:
   i) L⁴ and L⁵ are each independently a spacer moiety having the structure

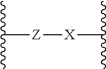

wherein:
wherein
The compound or pharmaceutically acceptable salt according to Embodiment 41, wherein D¹ and D² are independently selected from the group consisting of:
   X is a bond, triazolyl, or —CH₂—triazolyl-,
   wherein X is connected to R² or R³; or
   (ii) L⁴ and L⁵, independently, are a spacer moiety having the structure

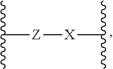

wherein:
Z is —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{b}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is —CH₂—triazolyl-C₁-C₄alkylene-OC(O)NHS(O)₂NH—, —C₄-C₆cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂—triazolyl-C₁-C₄alkylene-OC(O)NHS(O)₂NH—(CH₂CH₂O)ₙ—, —C₄-C₆cycloalkylene-OC(O)NHS(O)₂NH—(CH₂CH₂O)ₙ—, wherein each n independently is 1, 2, or 3, wherein X is connected to R² or R³.
Embodiment 75. The conjugate according to Embodiment 74, wherein Z is —O—, —CH₂NR^{L45}C(═O)—, —CH₂NR^{L45}C(═O)NH— or —CH₂O—; X is a bond, triazolyl, or —CH₂—triazolyl-; and R^{L45} in each occurrence, is independently H or C₁-C₃alkyl.
Embodiment 76. The conjugate according to any one of Embodiments 51 to 75, wherein L⁴ and L⁵ are each independently a spacer moiety selected from a group consisting of

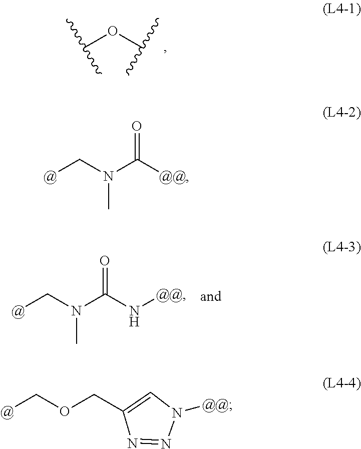

wherein the @ of L⁴ or L⁵ indicates the point of direct attachment to the phenyl group, and the @@ of L⁴ or L⁵ indicates the point of direct attachment to R² or R³.
Embodiment 77. The conjugate according to any one of Embodiments 51 to 76, wherein the hydrophilic groups represented by R² and R³ each independently comprises polyethylene glycol, polyalkylene glycol, a polyol, a polysarcosine, a sugar, an oligosaccharide, a polypeptide, C₂-C₆alkyl substituted with 1 to 3

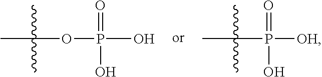 [SMILES: *OP(=O)(O)O ; "or" ; *P(=O)(O)O]

or C₂-C₆alkyl substituted with 1 to 2 substituents independently selected from —OC(═O)NHS(O)₂NHCH₂CH₂OCH₃, —NHC(═O)C₁-C₄alkylene-P(O)(OCH₂CH₃)₂ and —COOH groups.
Embodiment 78. The conjugate according to any one of Embodiments 51 to 76, wherein R² or/and R³ independently is/are each selected from the group consisting of:

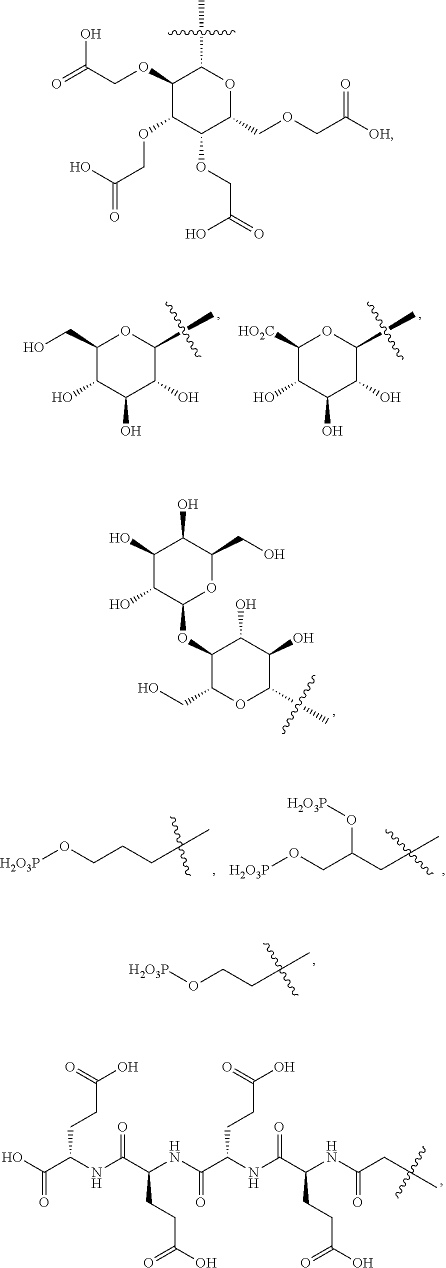

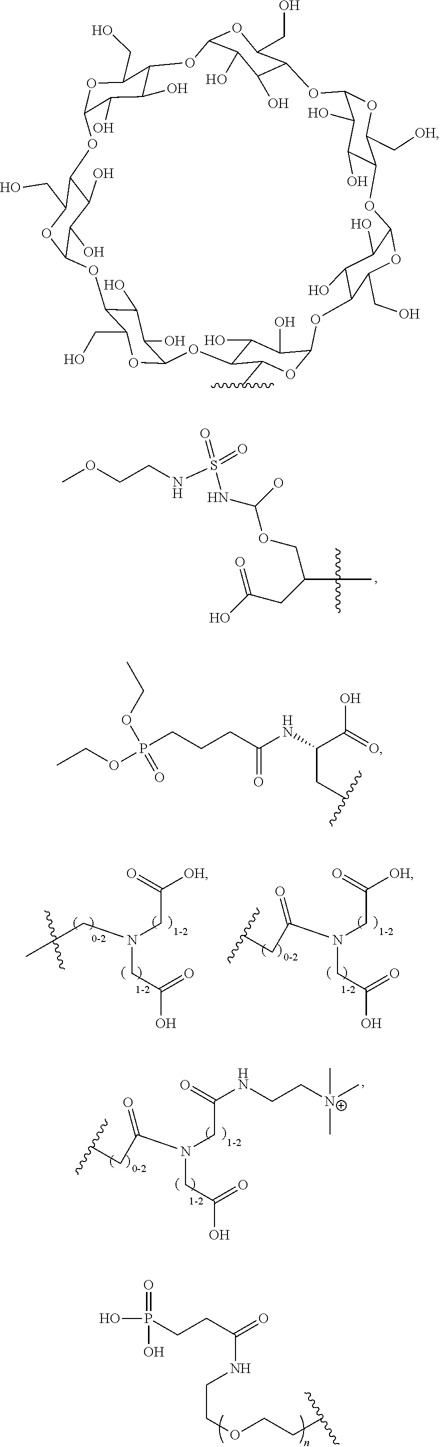

wherein
n is an integer between 1 and 6,

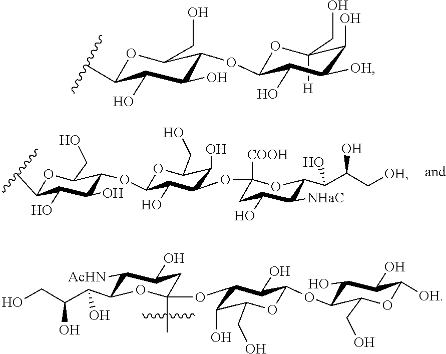

Embodiment 79. The conjugate according to any one of Embodiments 51 to 76, wherein R² or/and R³ is/are each independently comprising:
   (i) a polysarcosine with the following moiety:

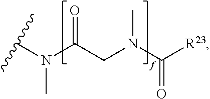

wherein
   f is an integer between 3 and 25; and
   R²³ is H, —CH₃ or —CH₂CH₂C(═O)OH;
(ii) a polyethylene glycol of formula:

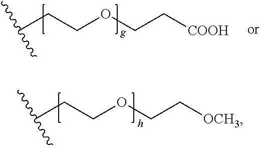

wherein
   g and h are independently an integer between 2 and 30; or
(iii) a moiety represented by

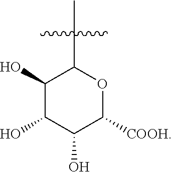 [SMILES: *C1O[C@H](C(=O)O)[C@H](O)[C@H](O)[C@H]1O]

Embodiment 79a. The conjugate according to any one of Embodiments 51 to 76, wherein the hydrophilic group represented by R² or R³ each independently comprises:
(i) a polysarcosine with the following moiety:

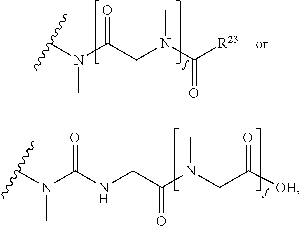

Wherein f is an integer between 3 and 25; and R²³ is H, —CH₃ or —CH₂CH₂C(═O)OH; or
(ii) a polyethylene glycol of formula:

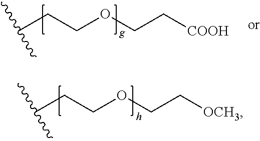

wherein g and h are independently an integer between 2 and 30;
or
(iii) a moiety represented by

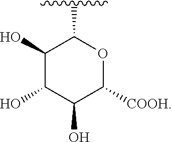 [SMILES: *[C@@H]1O[C@H](C(=O)O)[C@@H](O)[C@H](O)[C@H]1O]

Embodiment 80. The conjugate according to any one of Embodiments 51 to 76, wherein R² and R³, independently, is/are each selected from a group consisting of

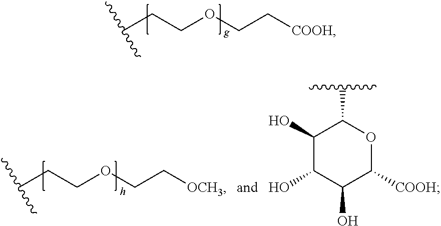

wherein
g and h are independently an integer between 20 and 30 (e.g. 23 or 24).
Embodiment 81. The conjugate thereof according to Embodiment 80, wherein R² or/and R³ comprises:

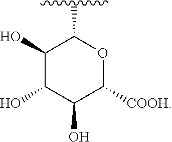 [SMILES: *[C@@H]1O[C@H](C(=O)O)[C@@H](O)[C@H](O)[C@H]1O]

Embodiment 81a. The conjugate according to Embodiment 80, wherein R² and R³ each independently comprises a polyethylene glycol of formula:

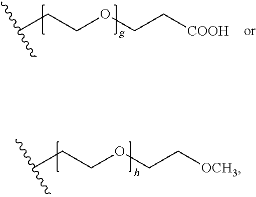

wherein g and h are independently an integer between 2 and 30.
Embodiment 82. The conjugate according to any one of Embodiments 46m to 81a, wherein D¹ and D² are the same.
Embodiment 83. The conjugate according to any one of Embodiments 46m to 81a, wherein D¹ and D² are different.
Embodiment 84. The conjugate according to any one of the Embodiments 46m to 83, wherein D¹ and D² are each independently selected from a cytotoxic drug, a cytostatic drug and an immunosuppressive drug.
Embodiment 85. The conjugate according to any one of the Embodiments 46m to 83, wherein D¹ and D² are each independently selected from an auristatin, a camptothecin, a duocarmycin, an etoposide, a maytansine, a maytansinoid, a taxane, a benzodiazepine or benzodiazepine containing drug (e.g., pyrrolo[1,4]-benzodiazepines (PBDs), indolinobenzodiazepines, and oxazolidinobenzodiazepines), and a vinca alkaloid.
Embodiment 85a. The conjugate according to Embodiment 85, wherein D¹ and D² are independently selected from the group consisting of:

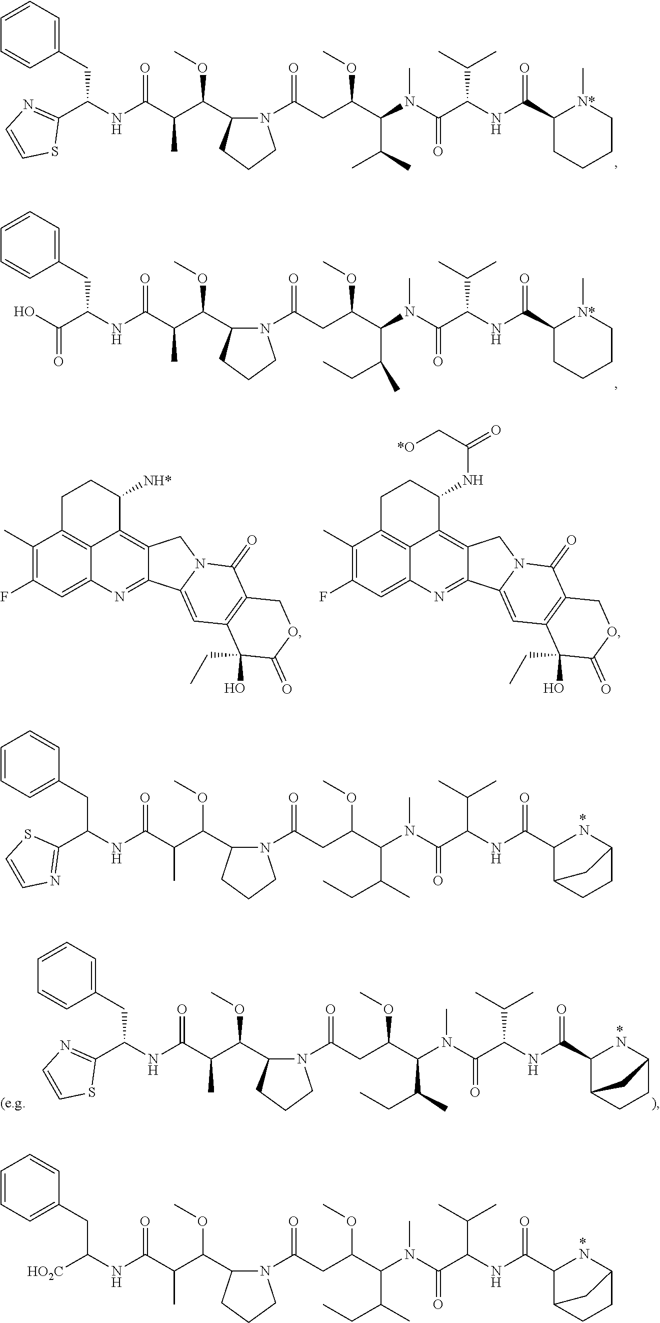

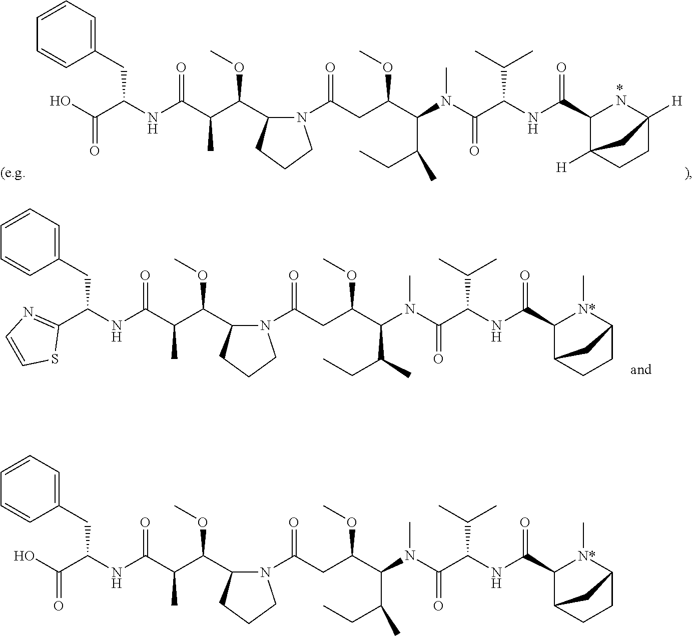

wherein * indicates the point of attachment to A¹ or A²; or a pharmaceutically acceptable salt thereof.
Embodiment 85b. The conjugate according to Embodiment 85a, wherein

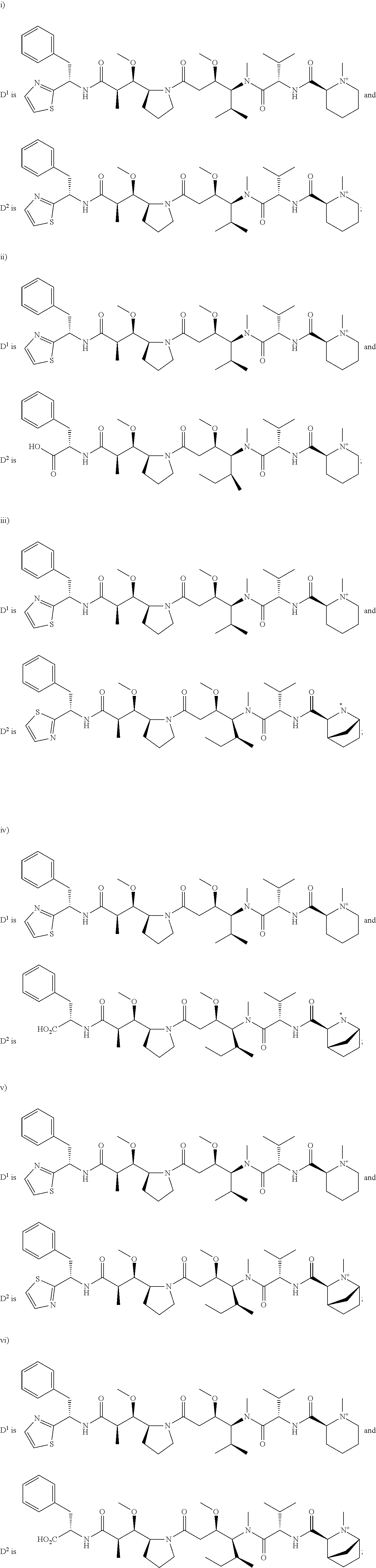

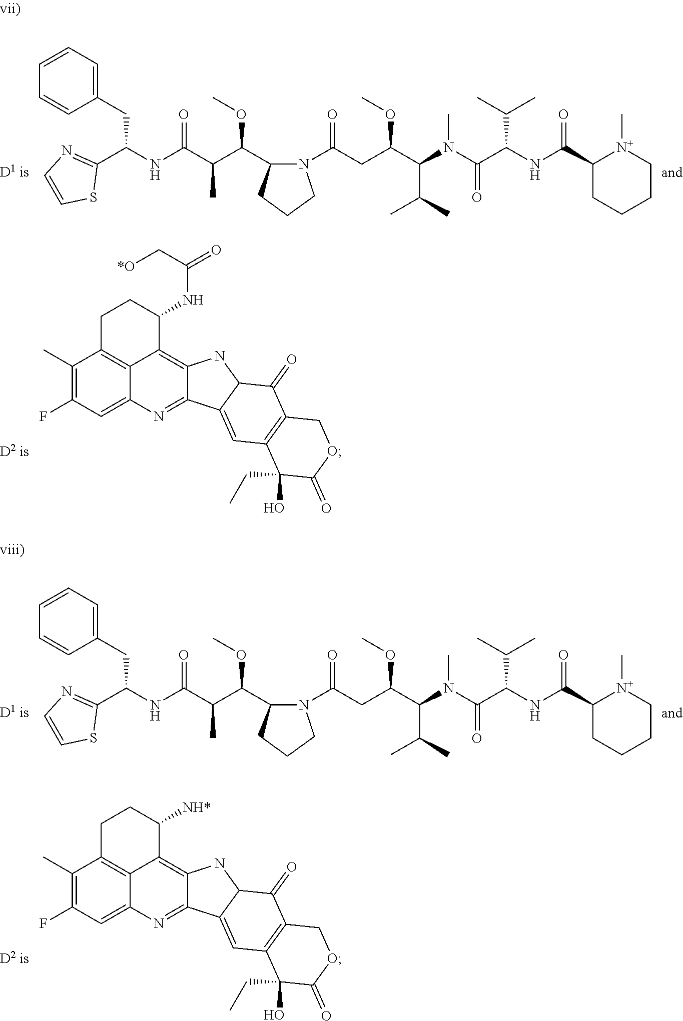

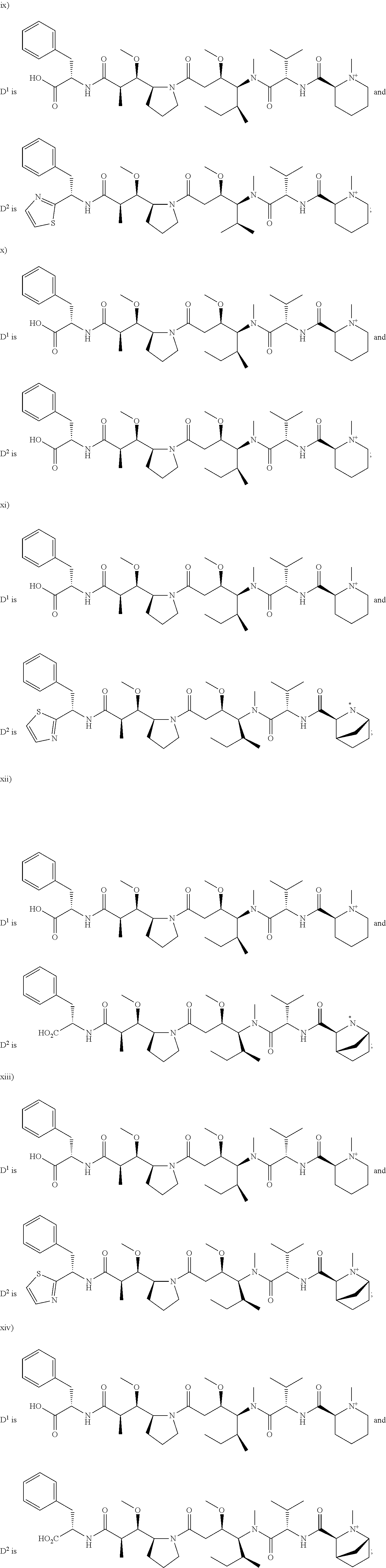

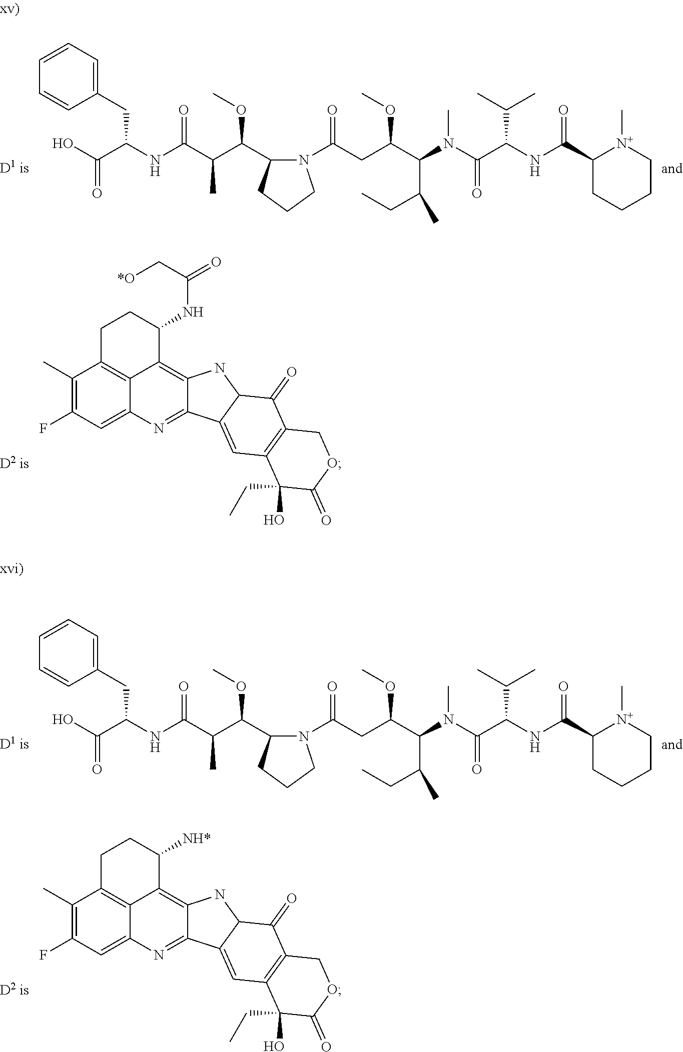

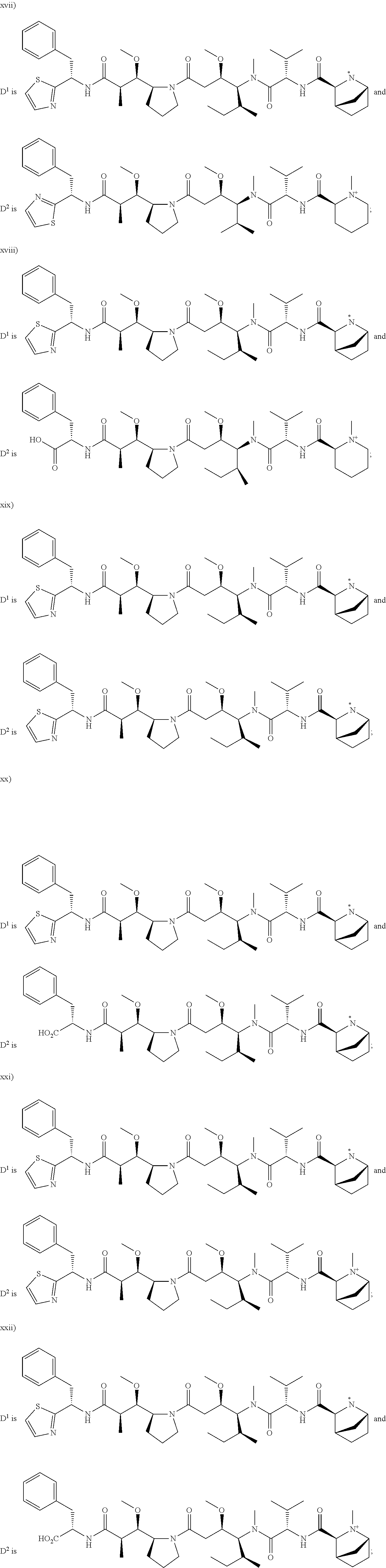

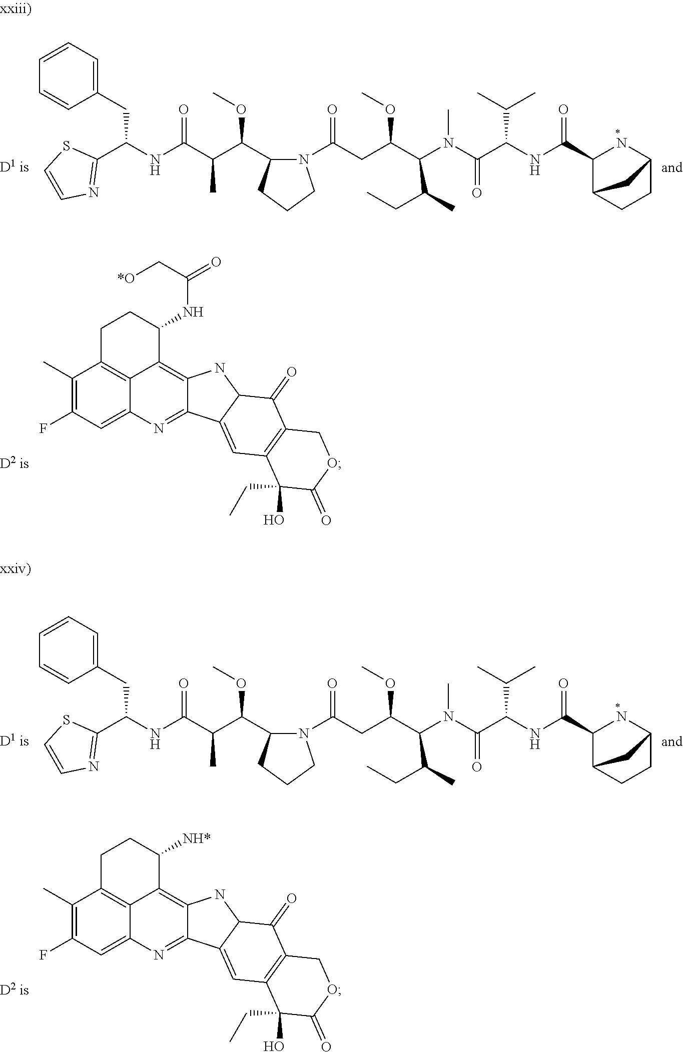

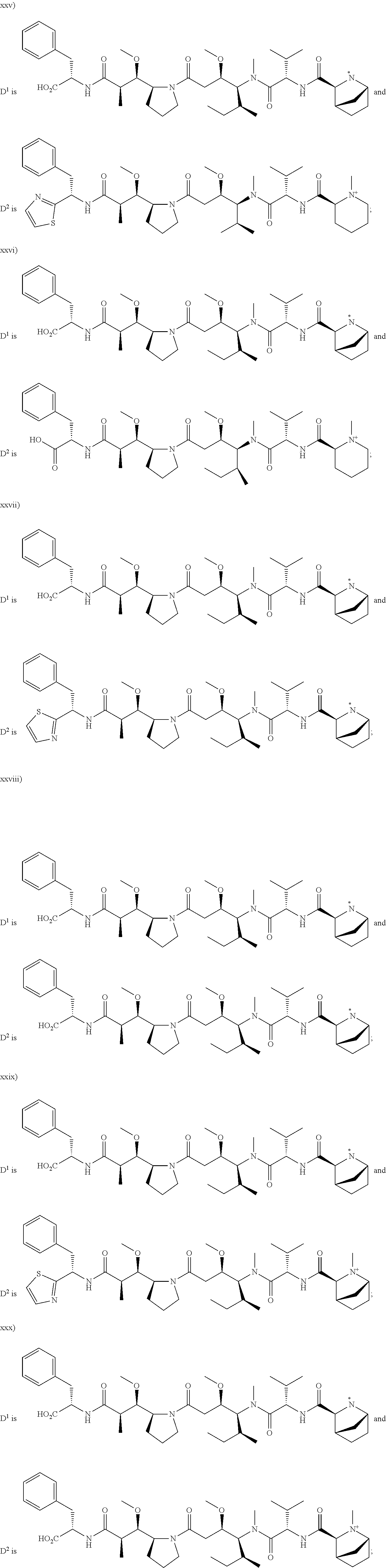

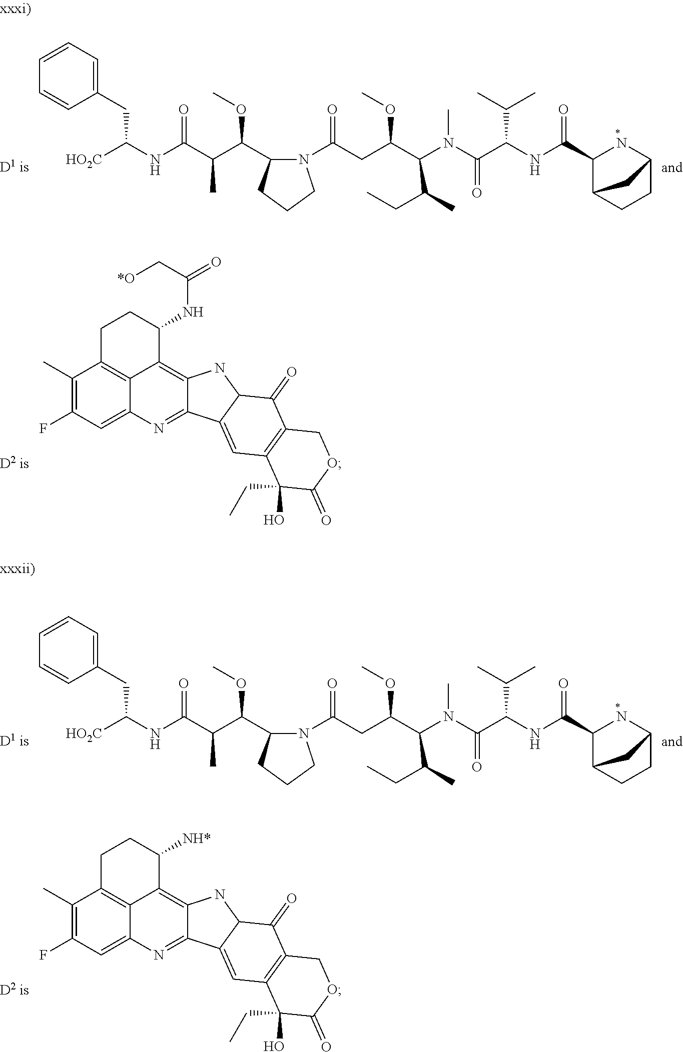

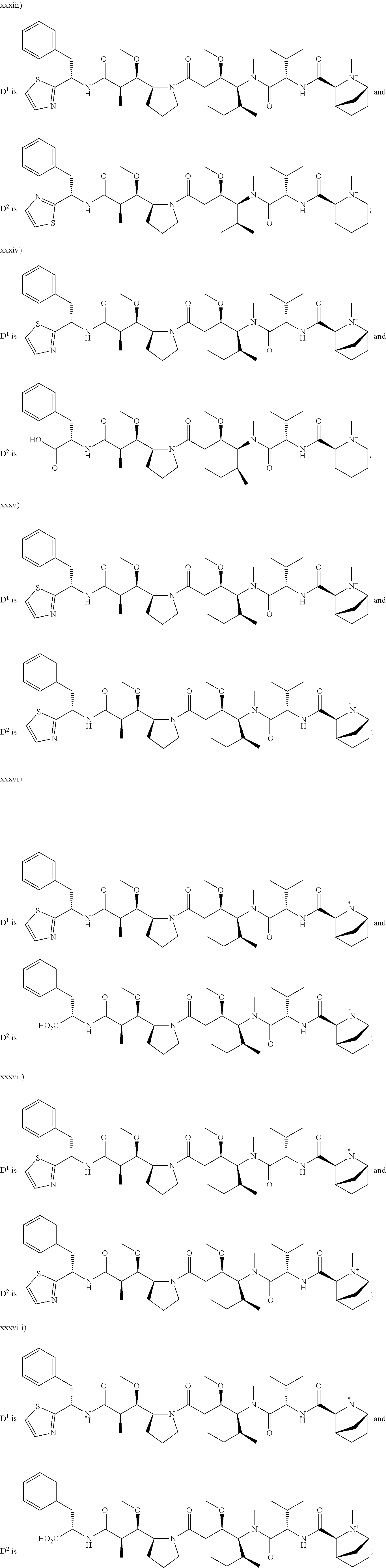

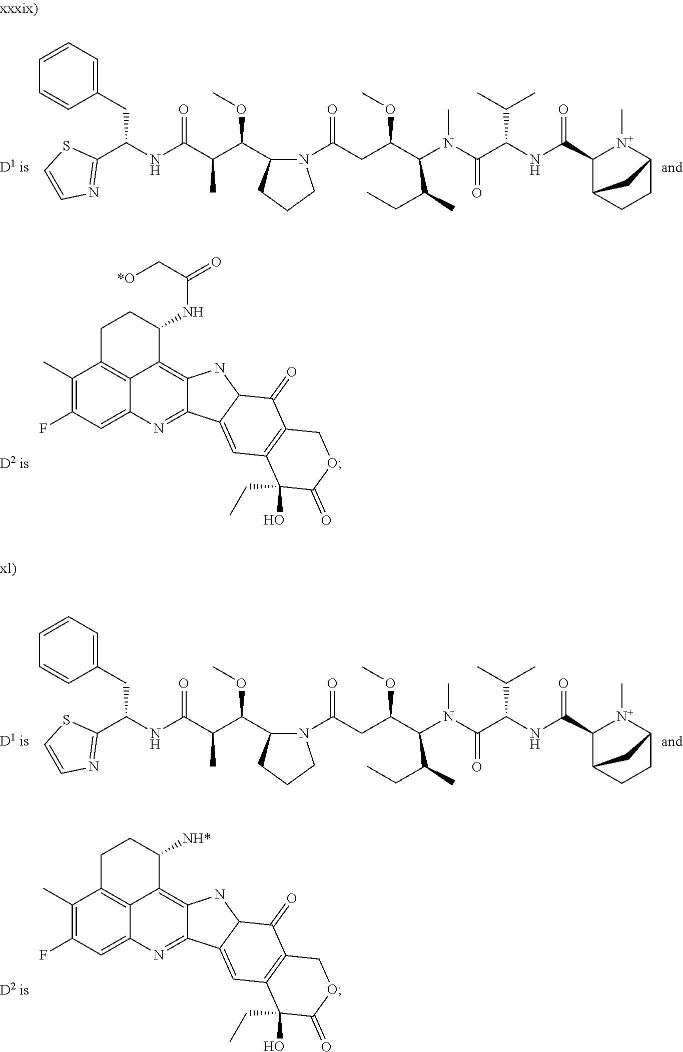

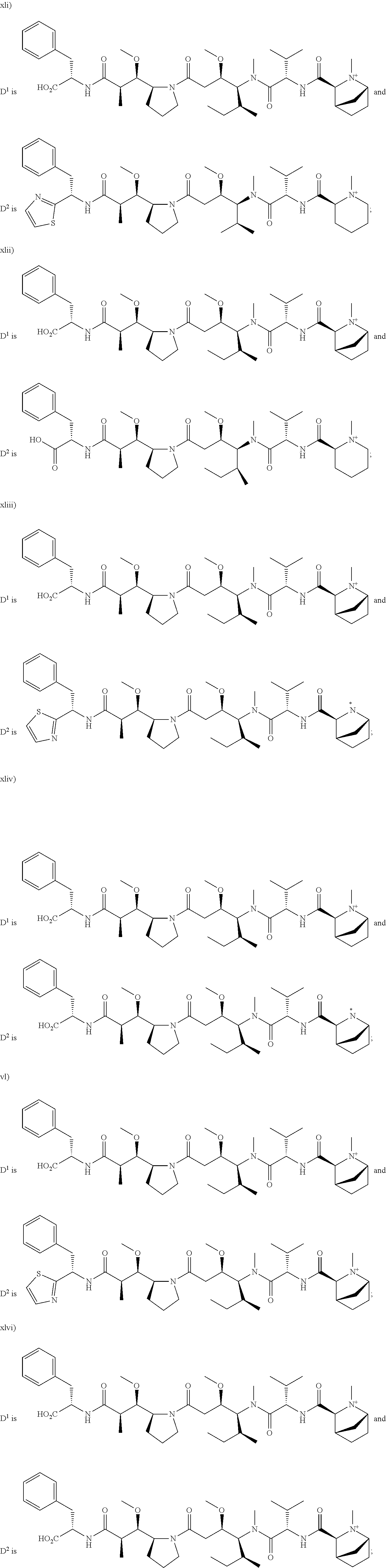

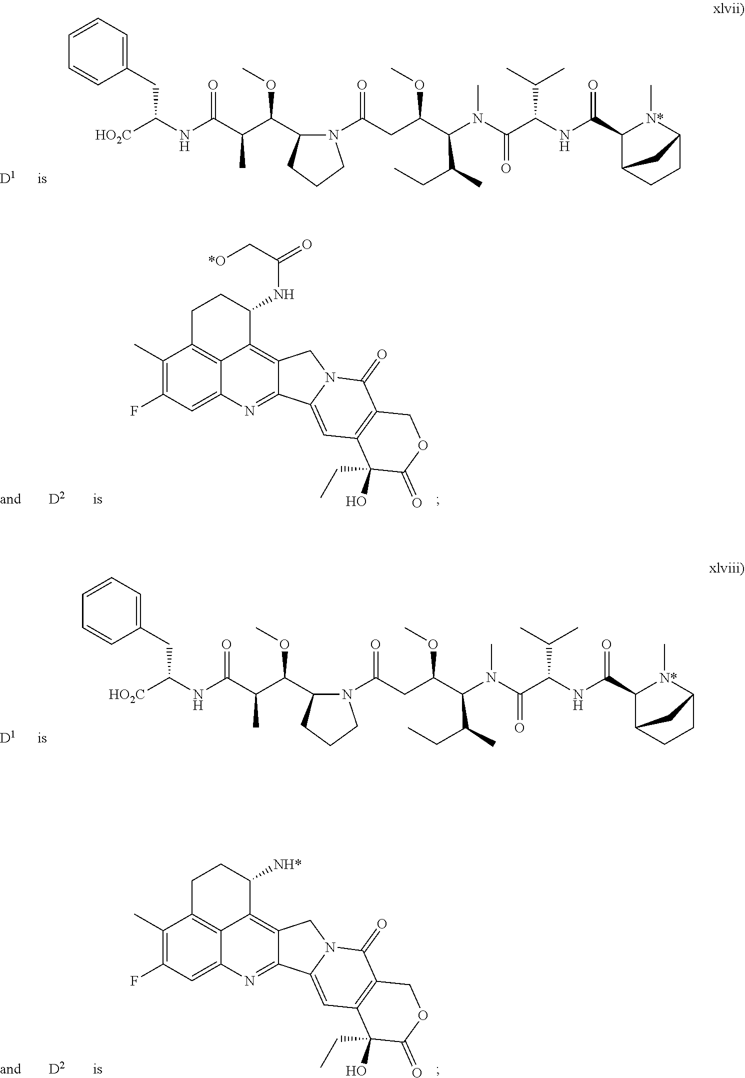

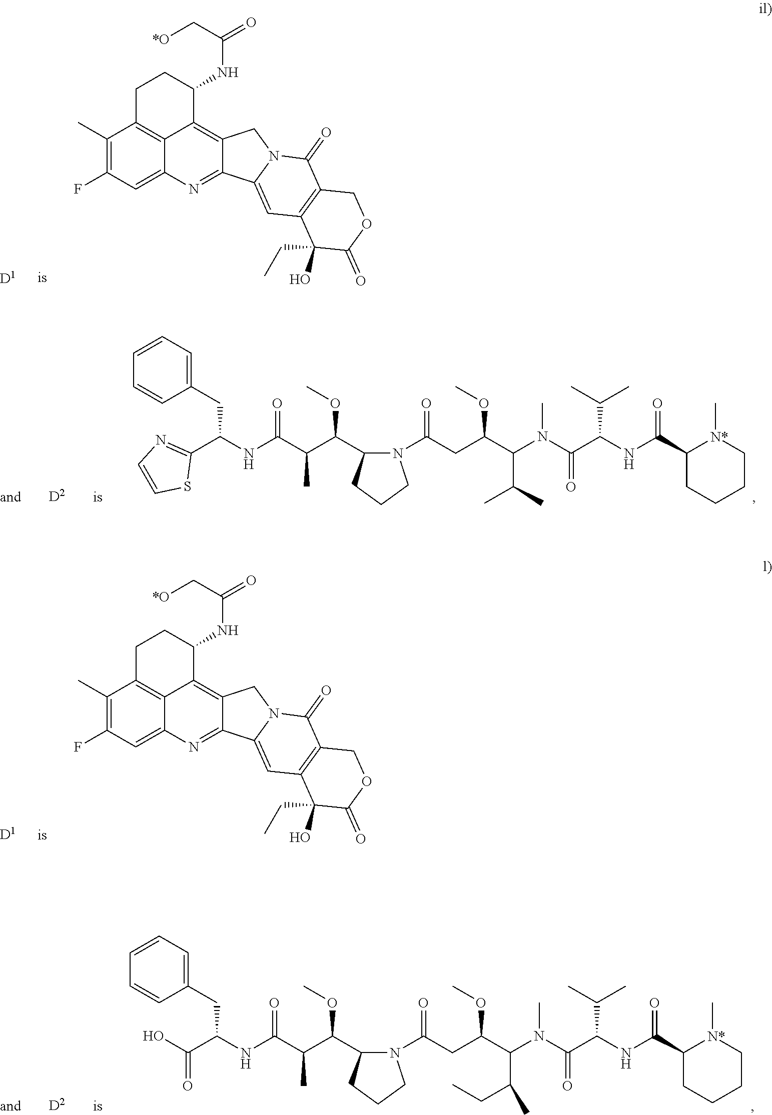

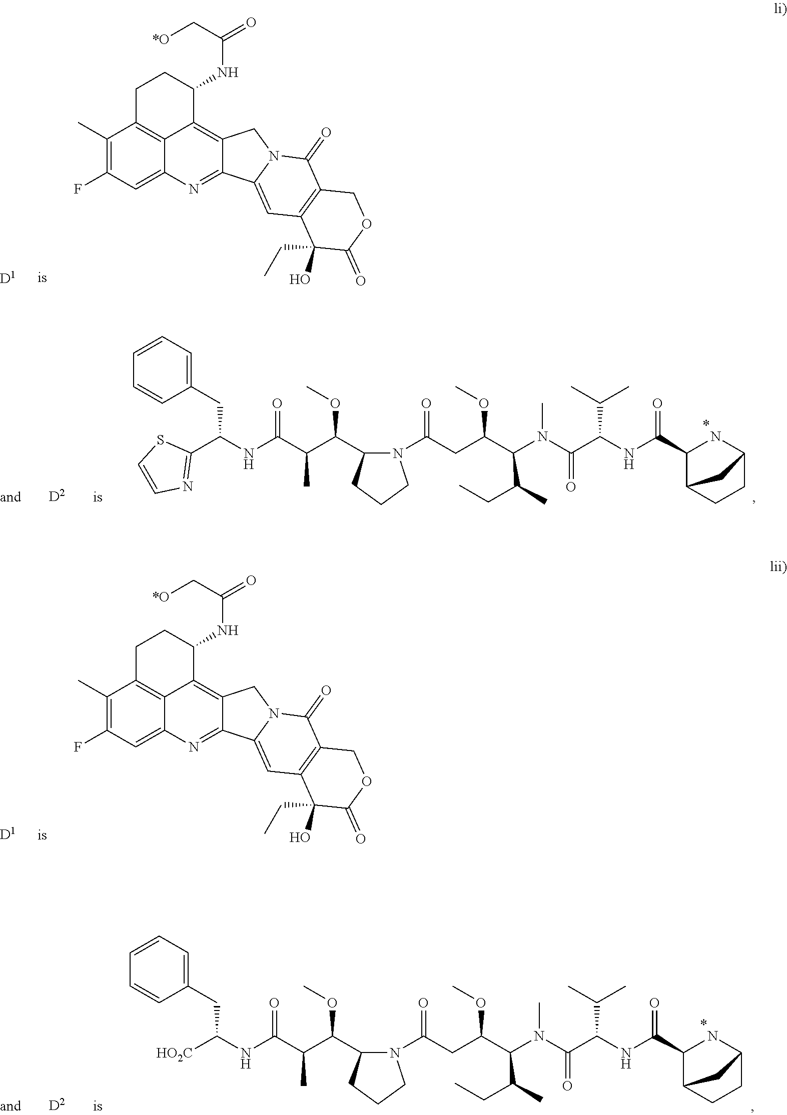

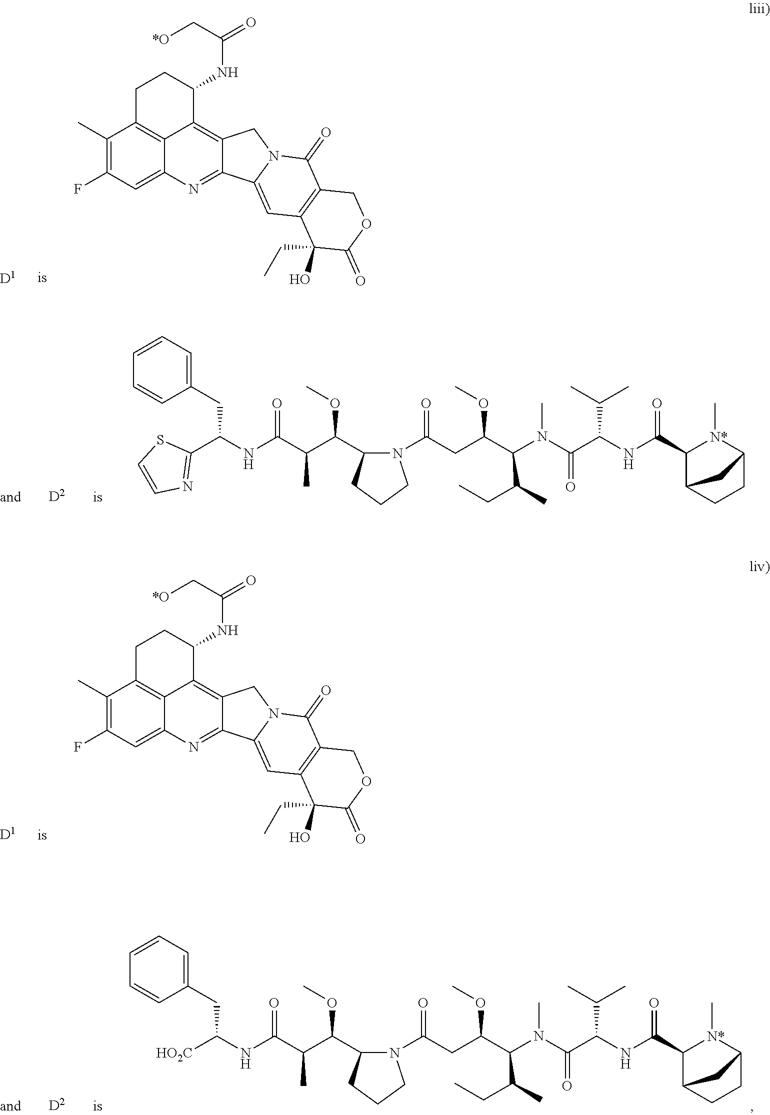

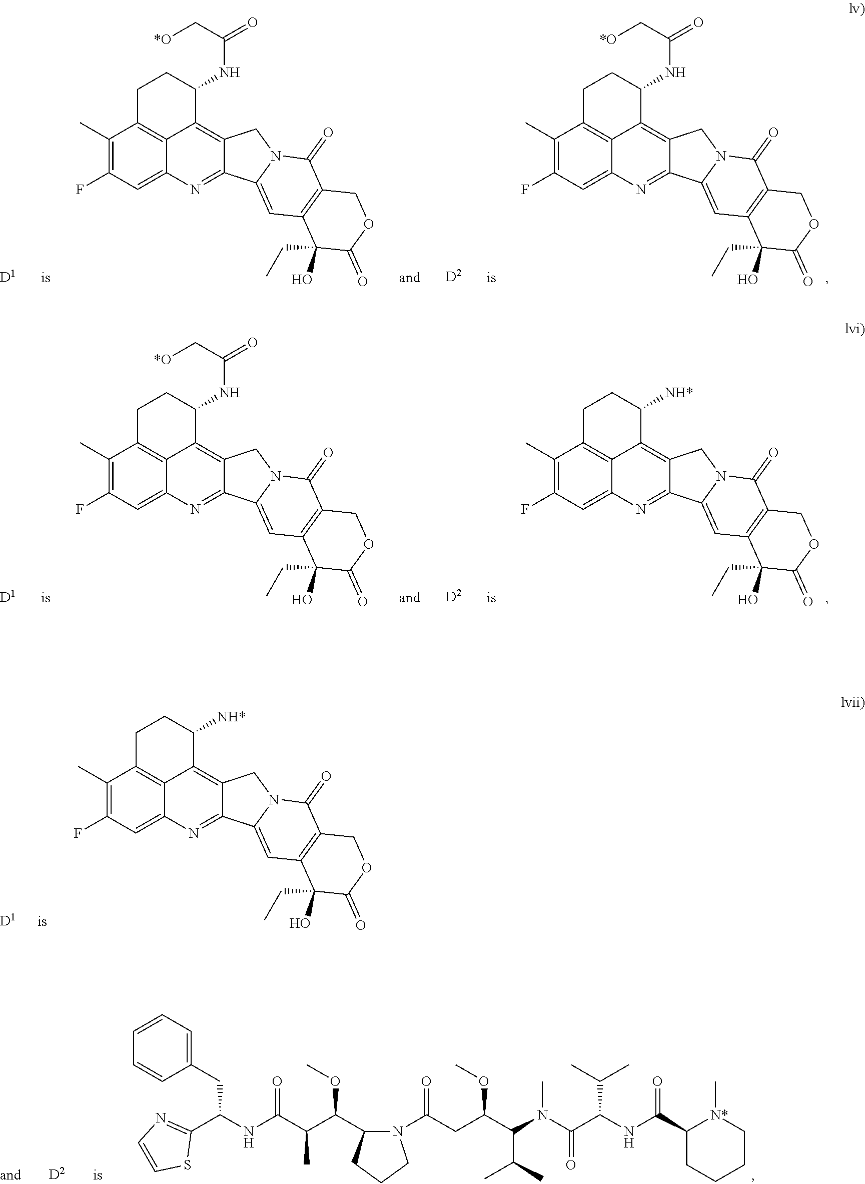

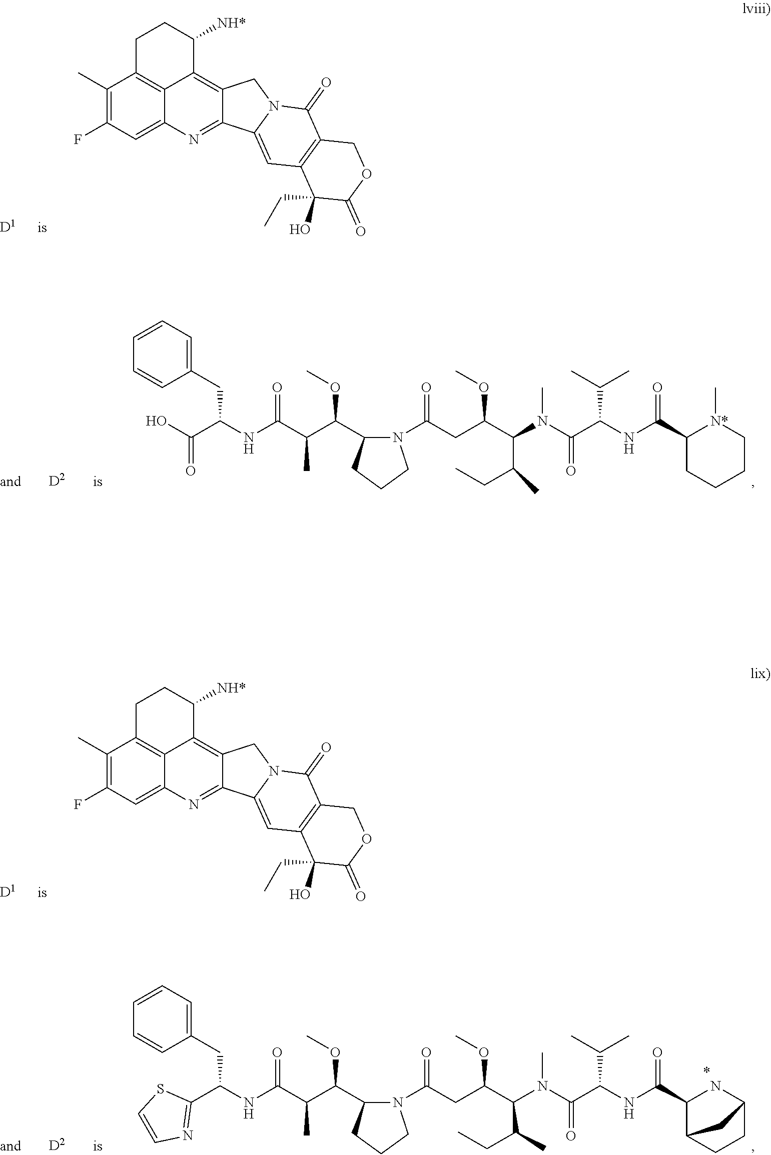

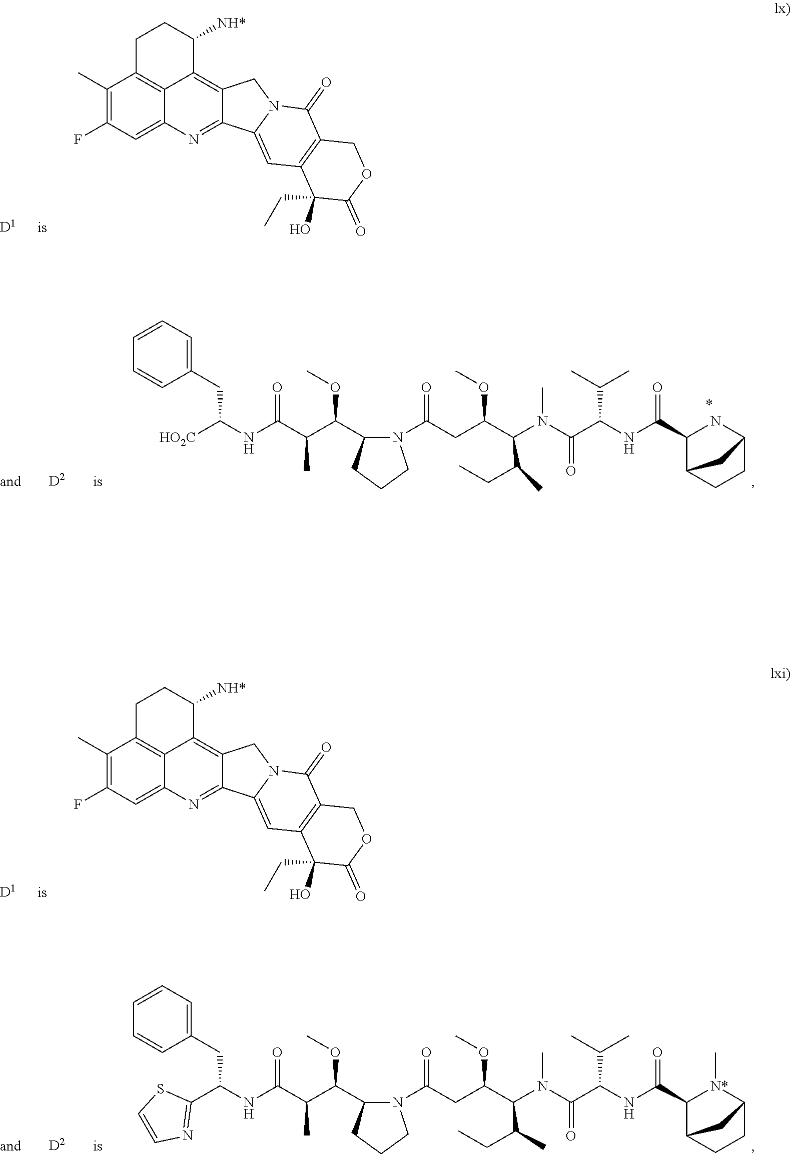

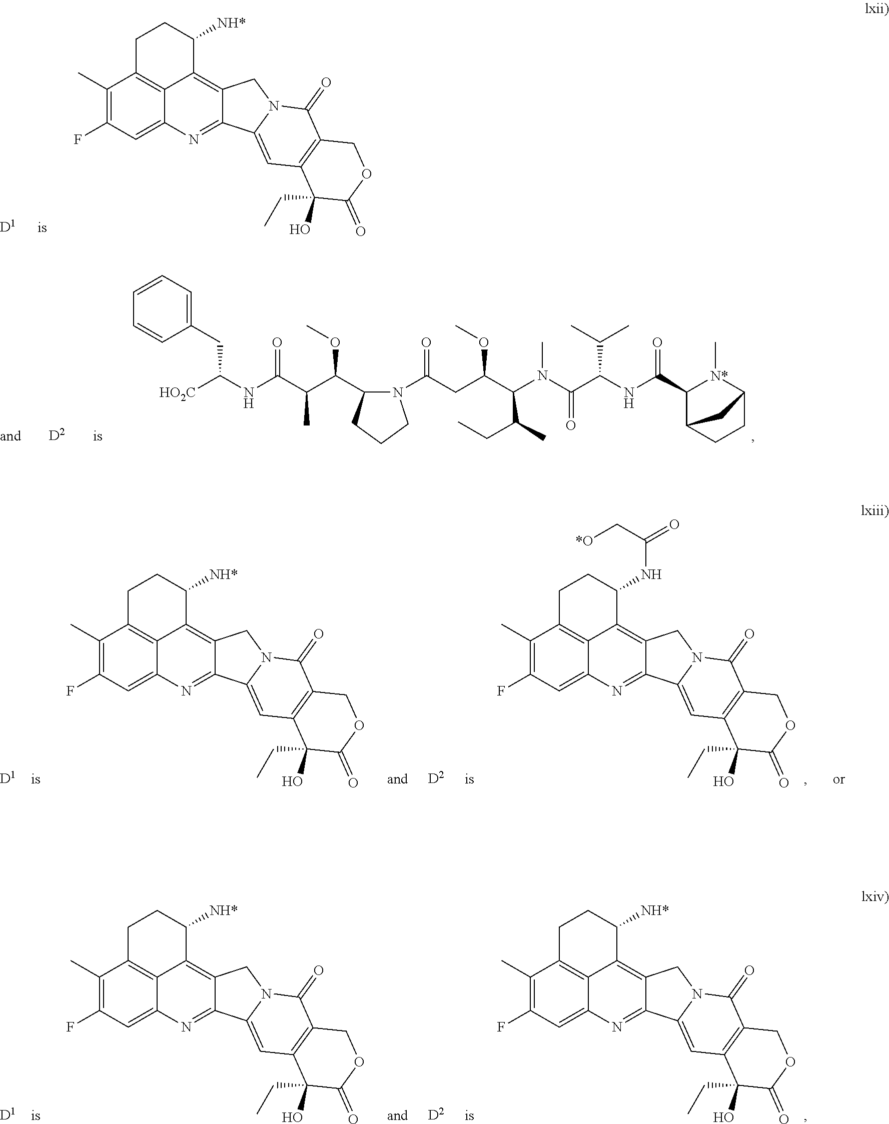

wherein * indicates the point of connection to the remainder of the molecule.
Embodiment 85c. The conjugate according to any one of Embodiments 46 to 85b, wherein:

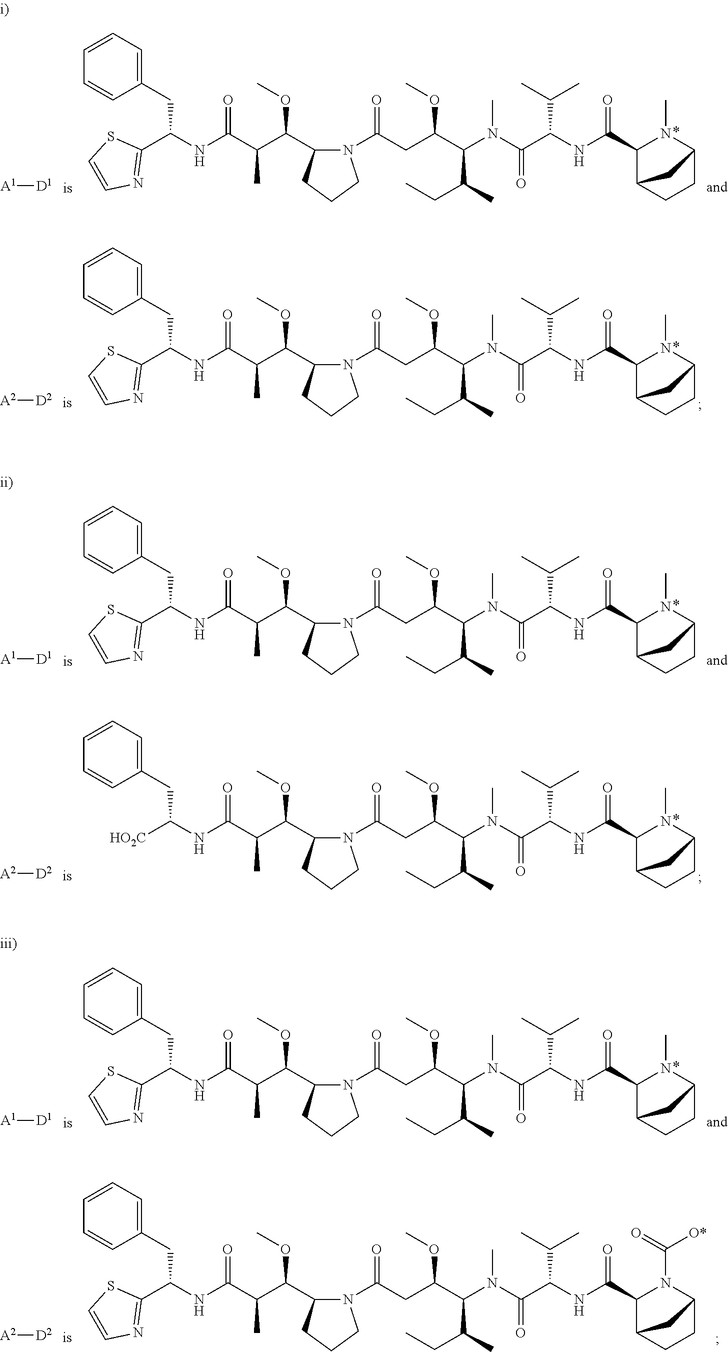

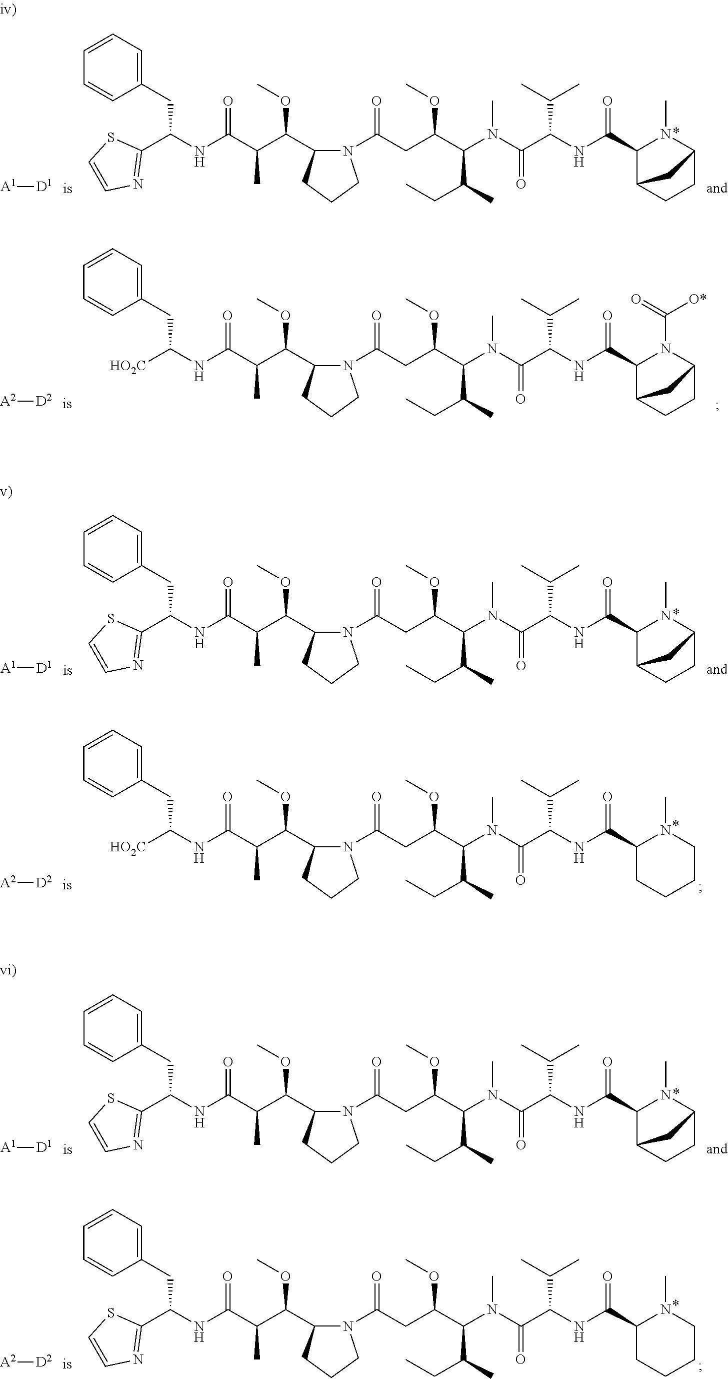

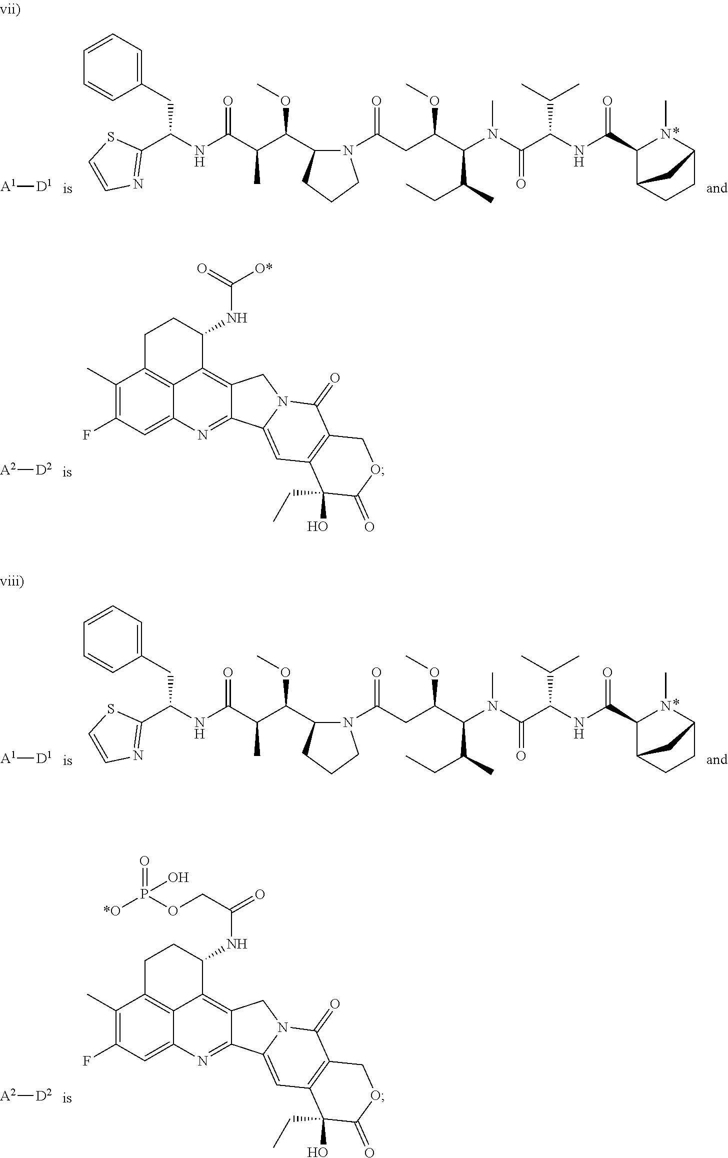

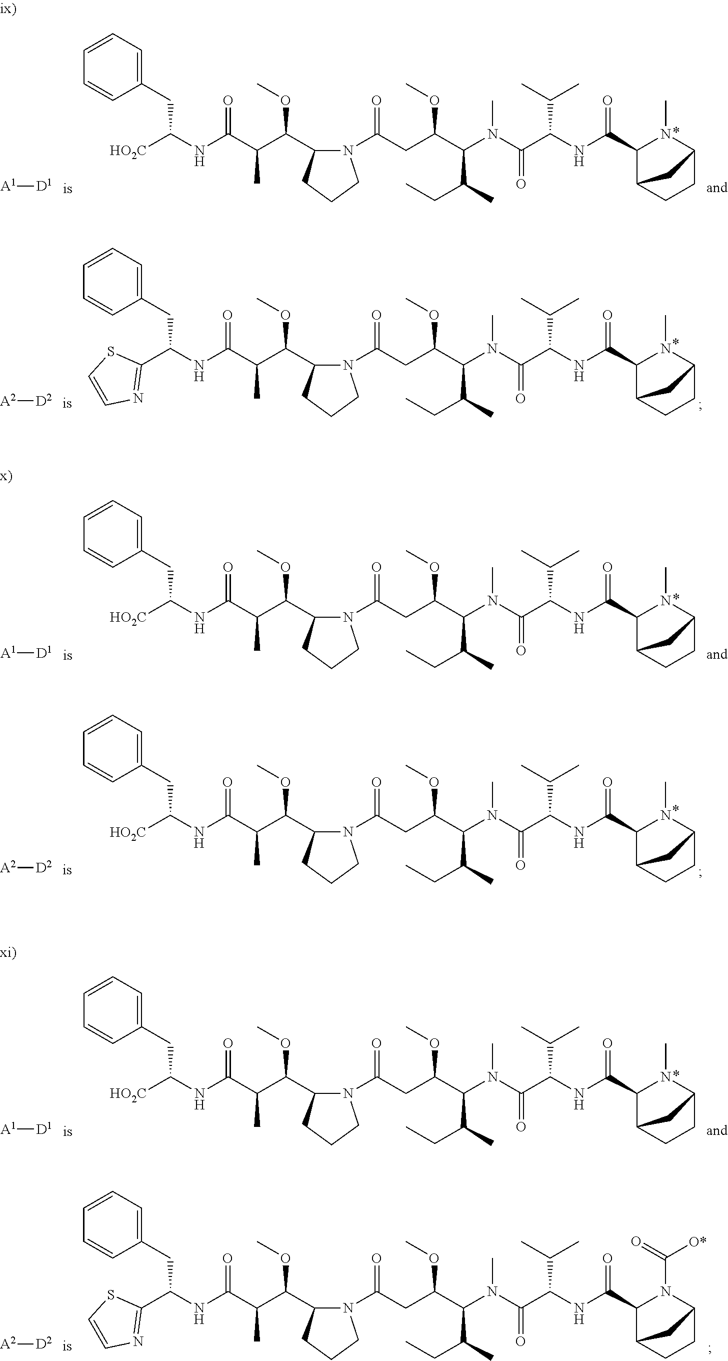

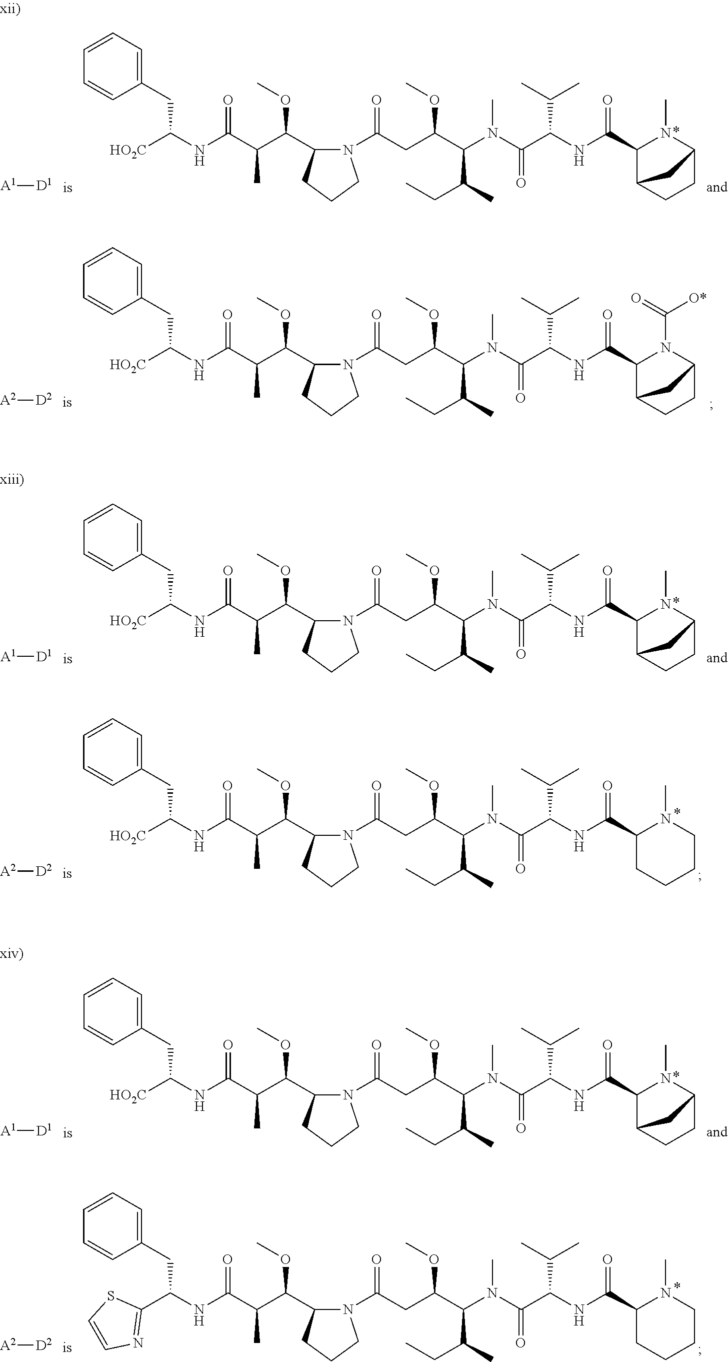

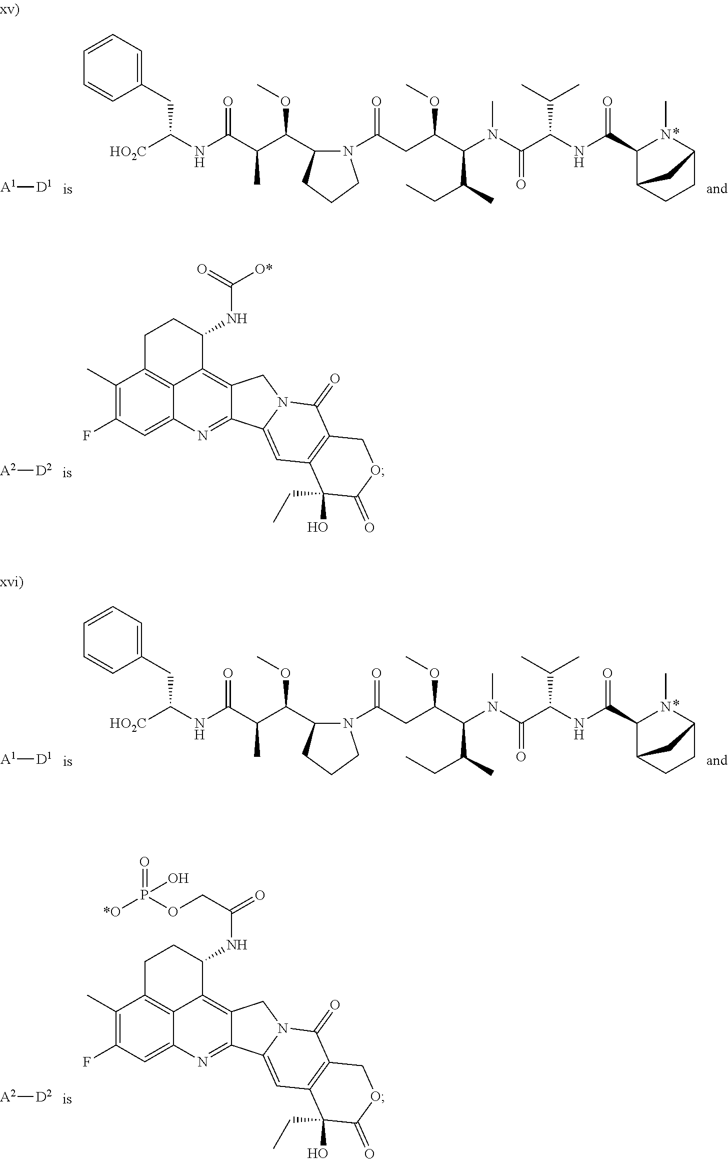

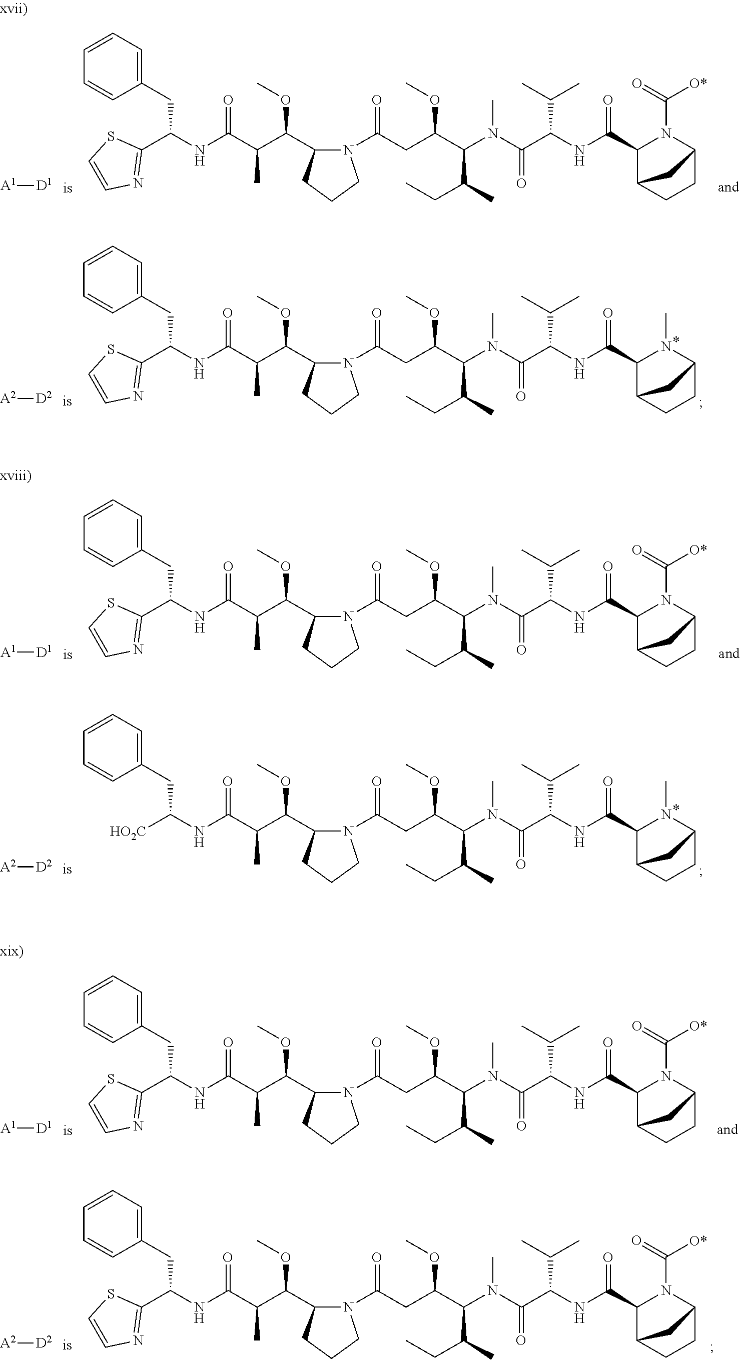

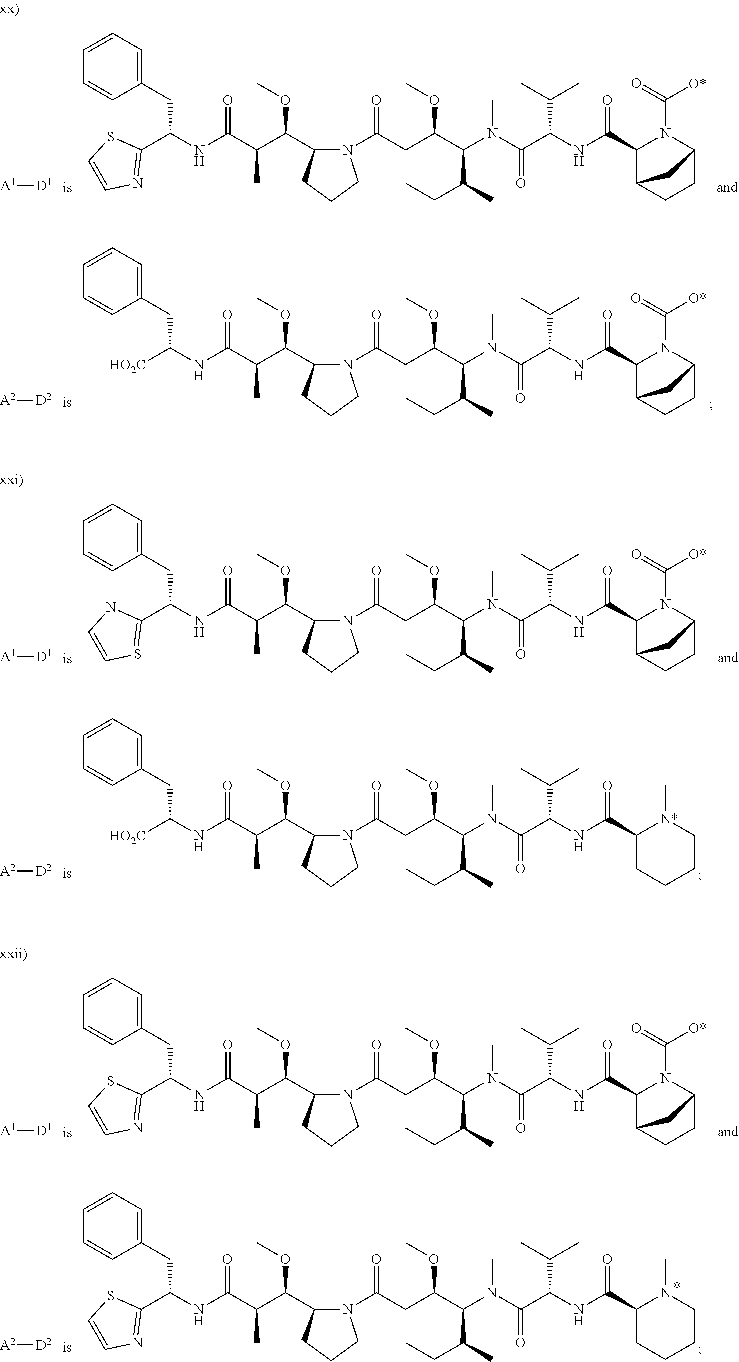

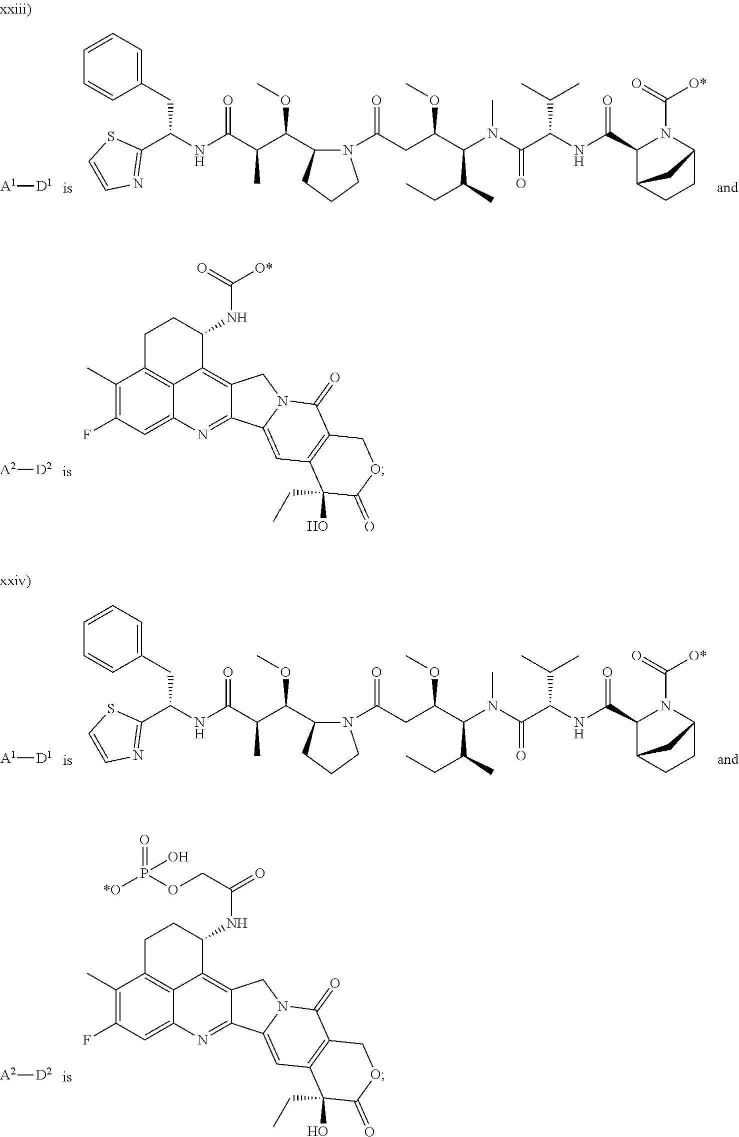

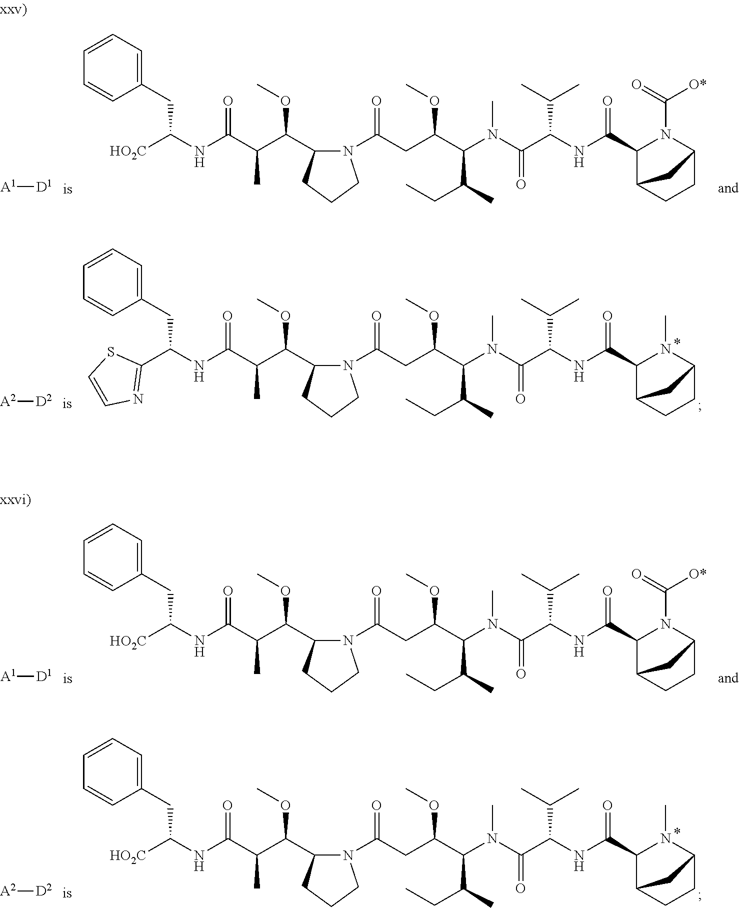

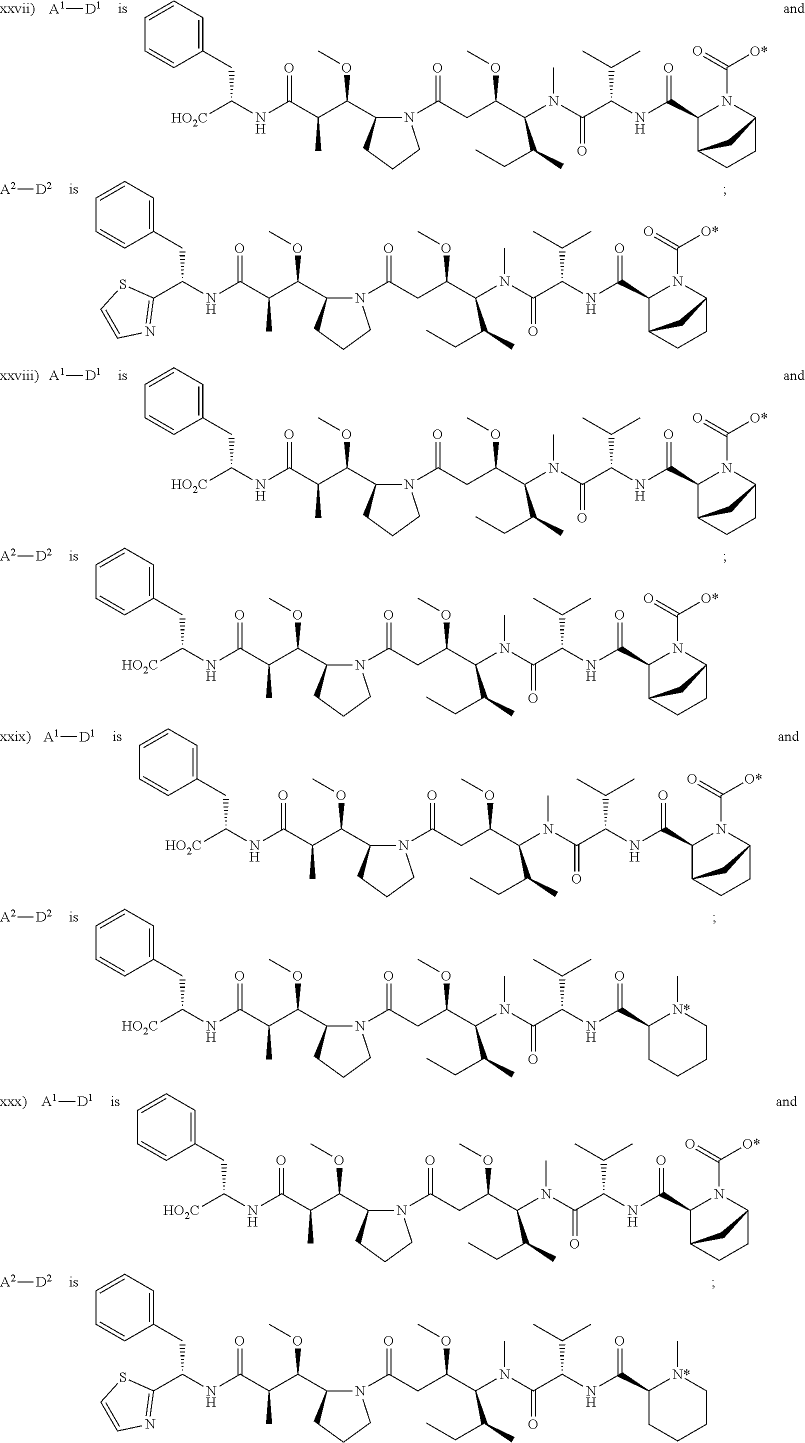

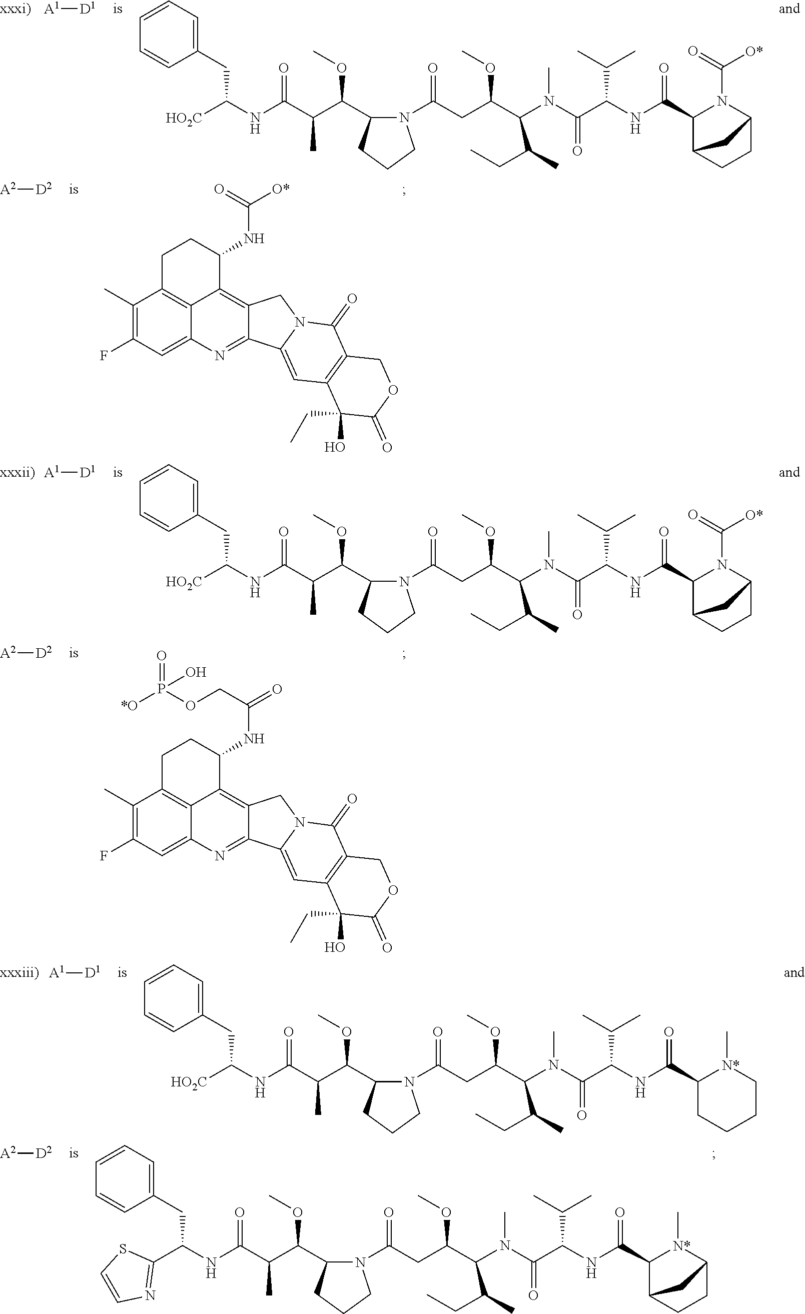

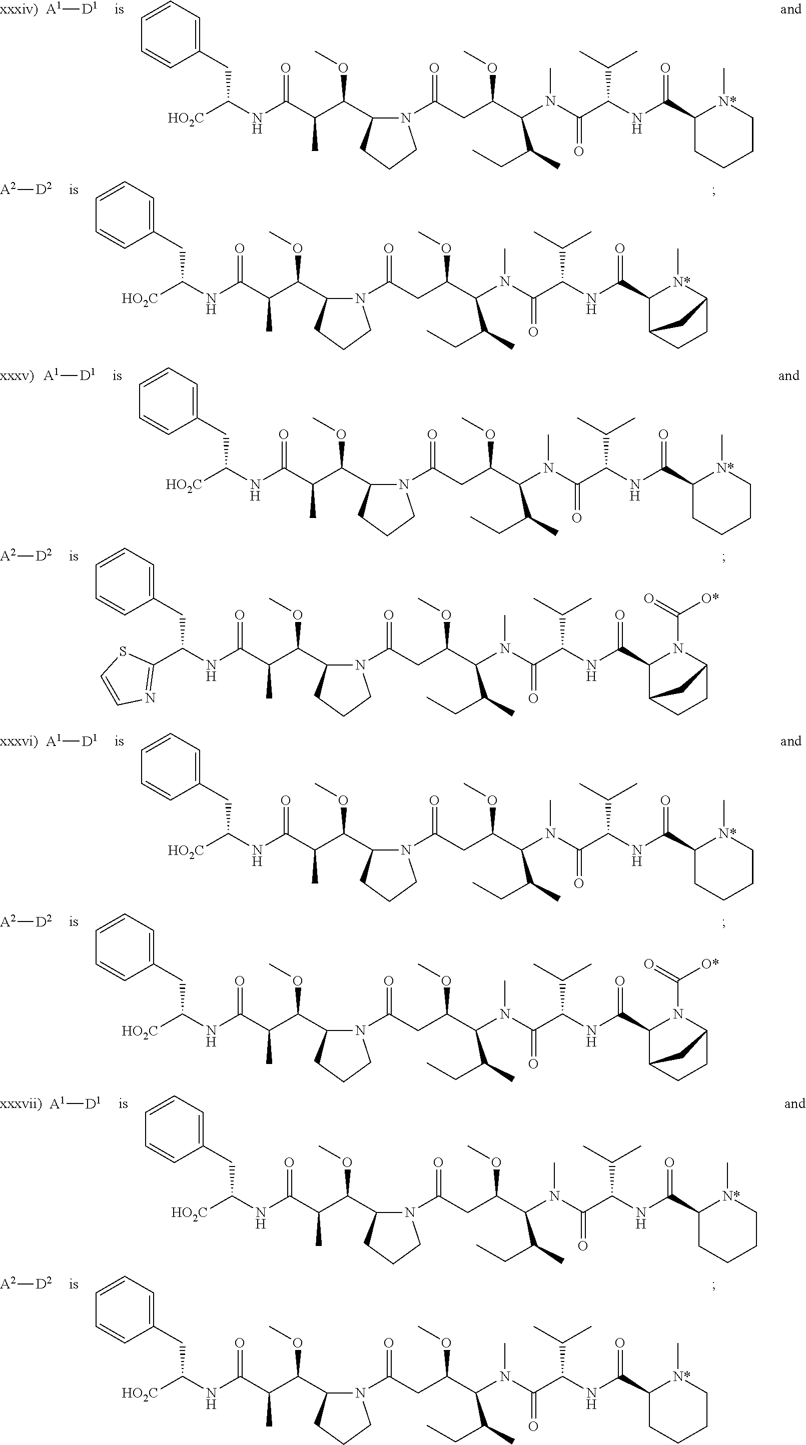

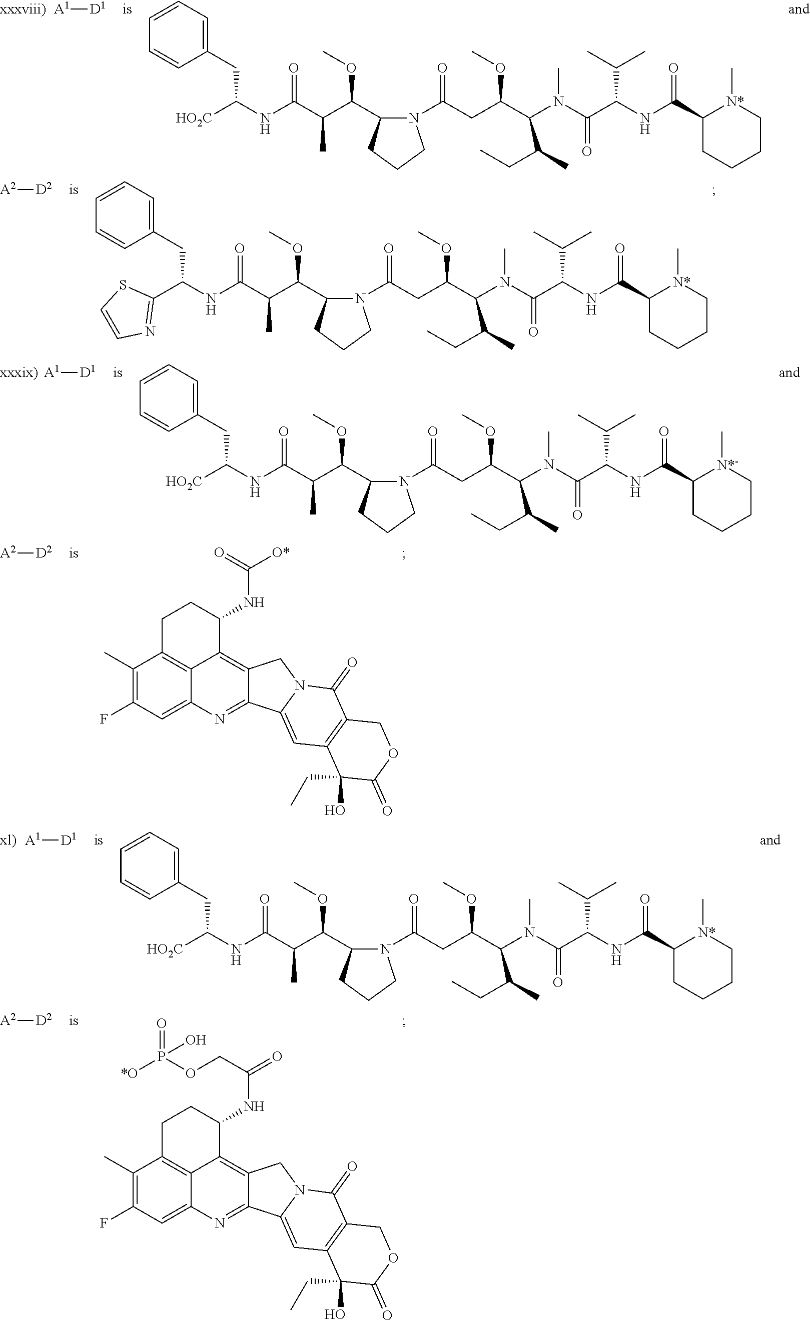

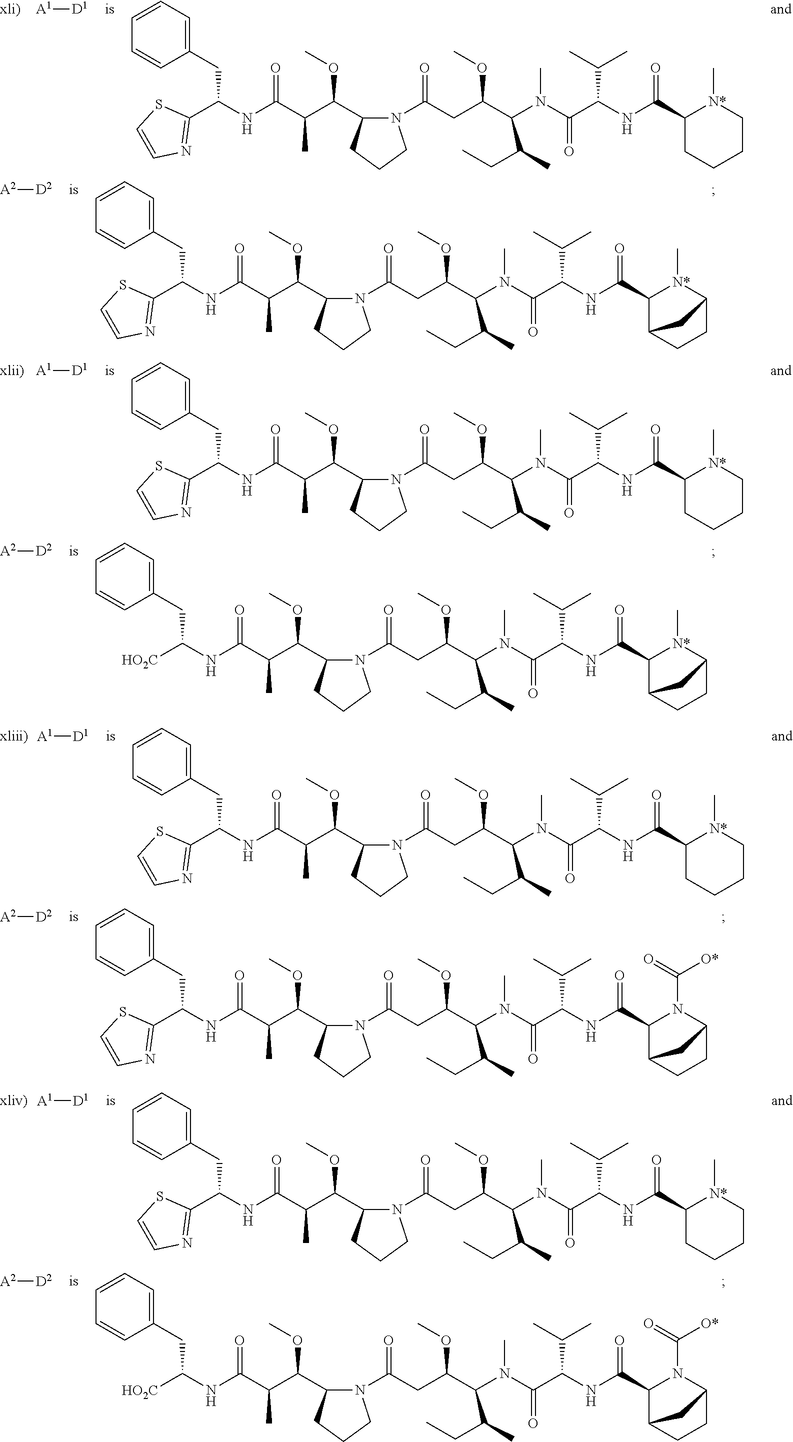

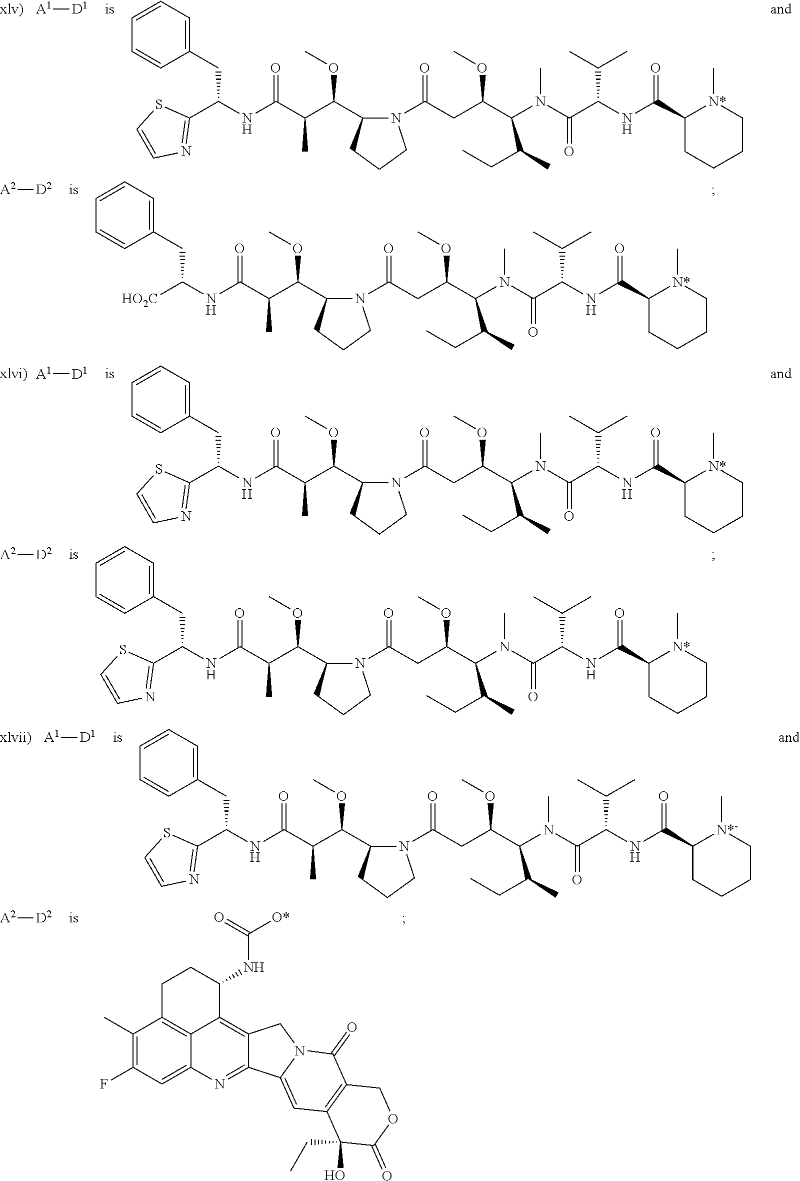

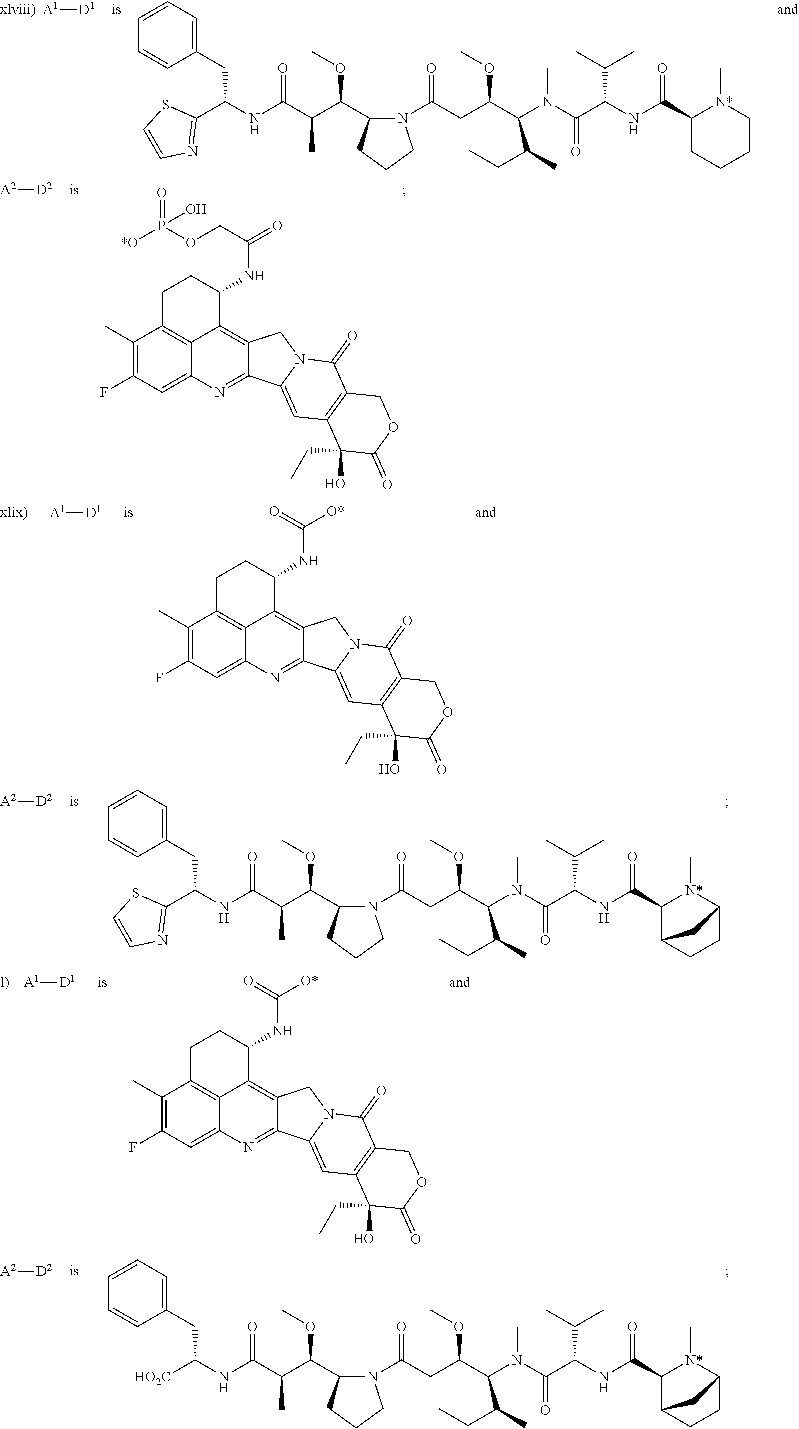

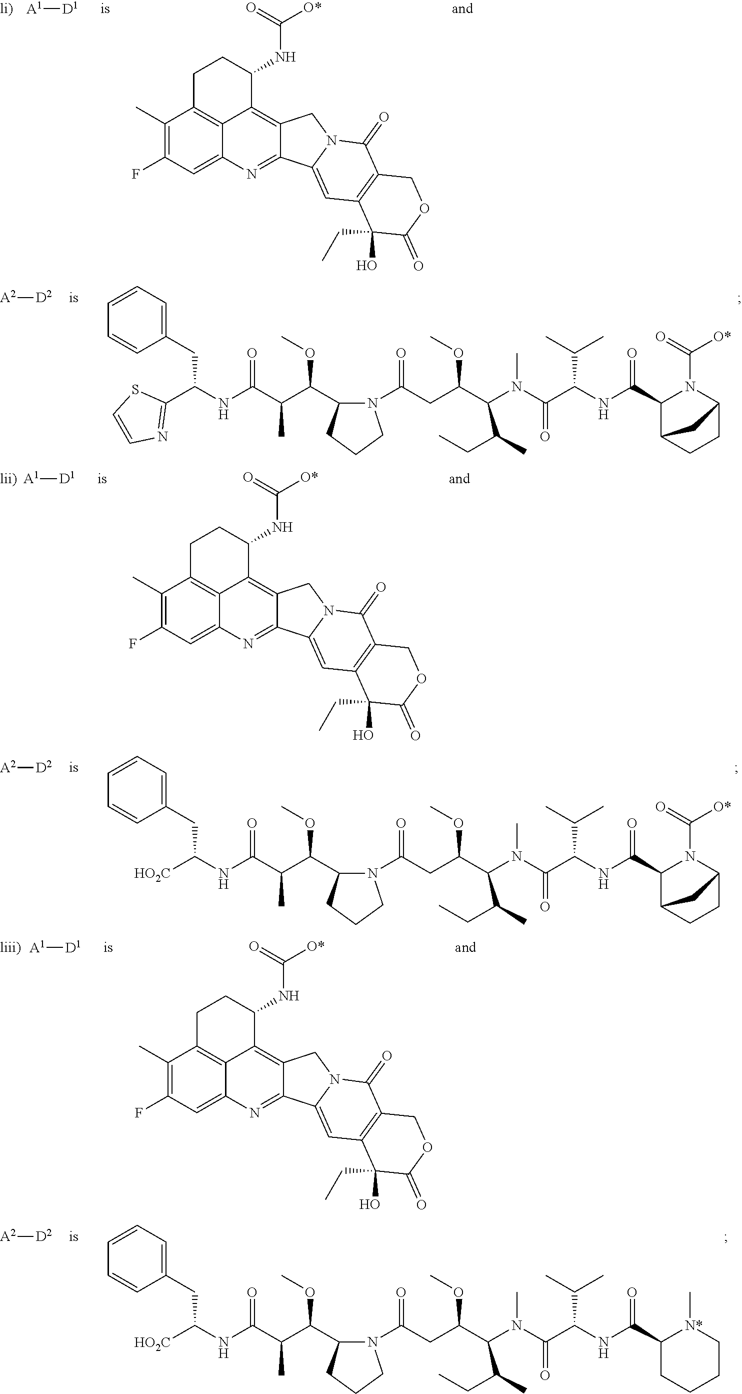

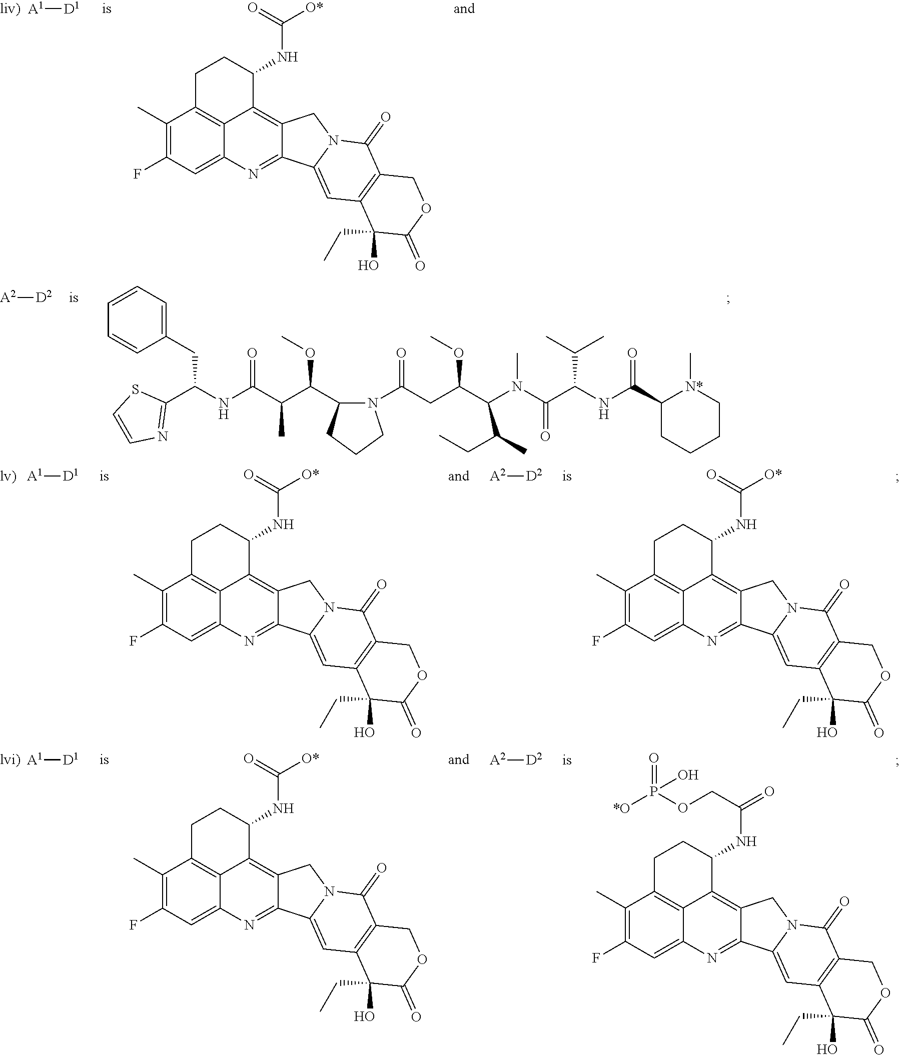

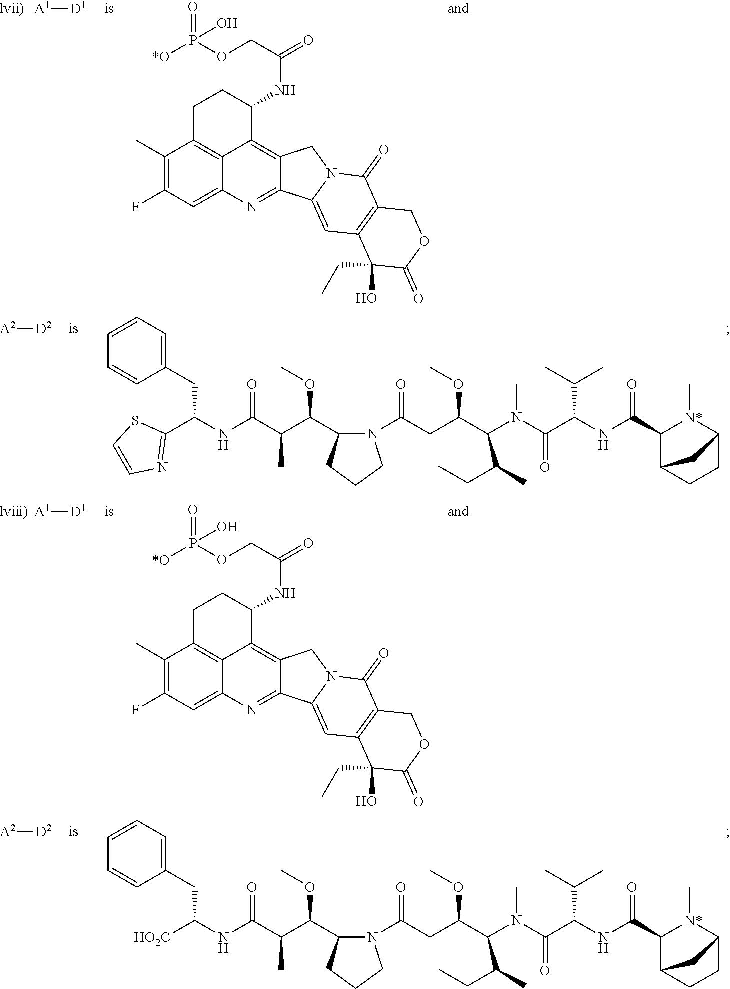

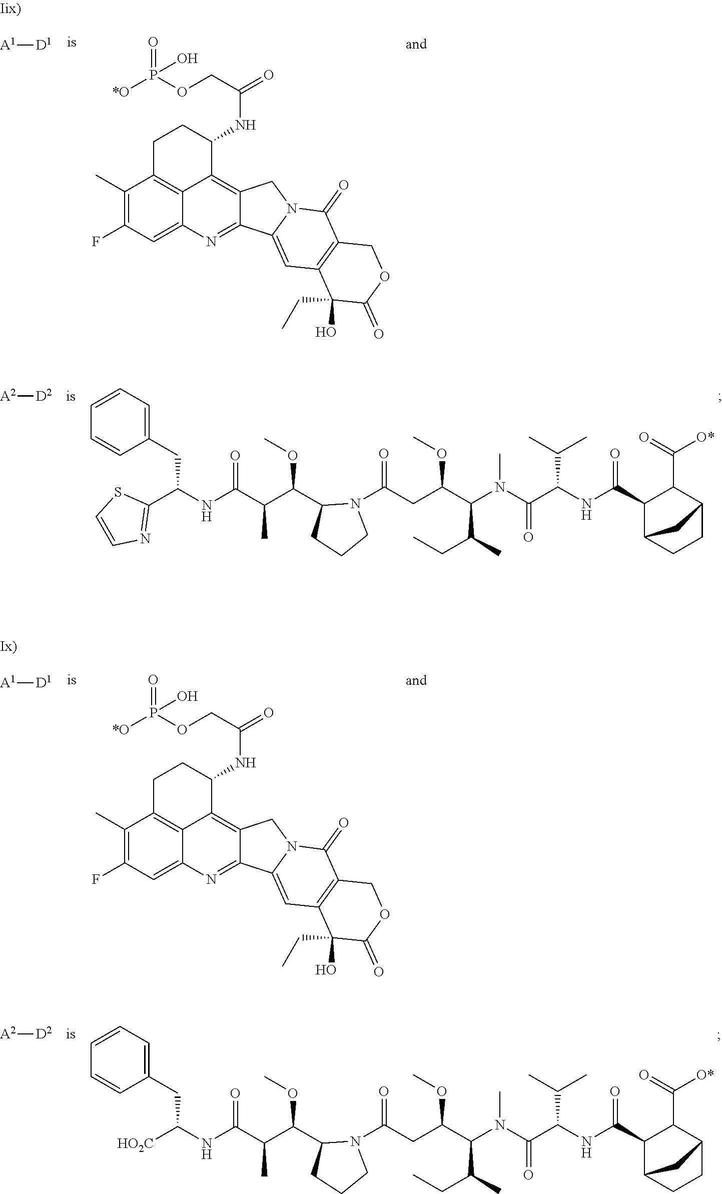

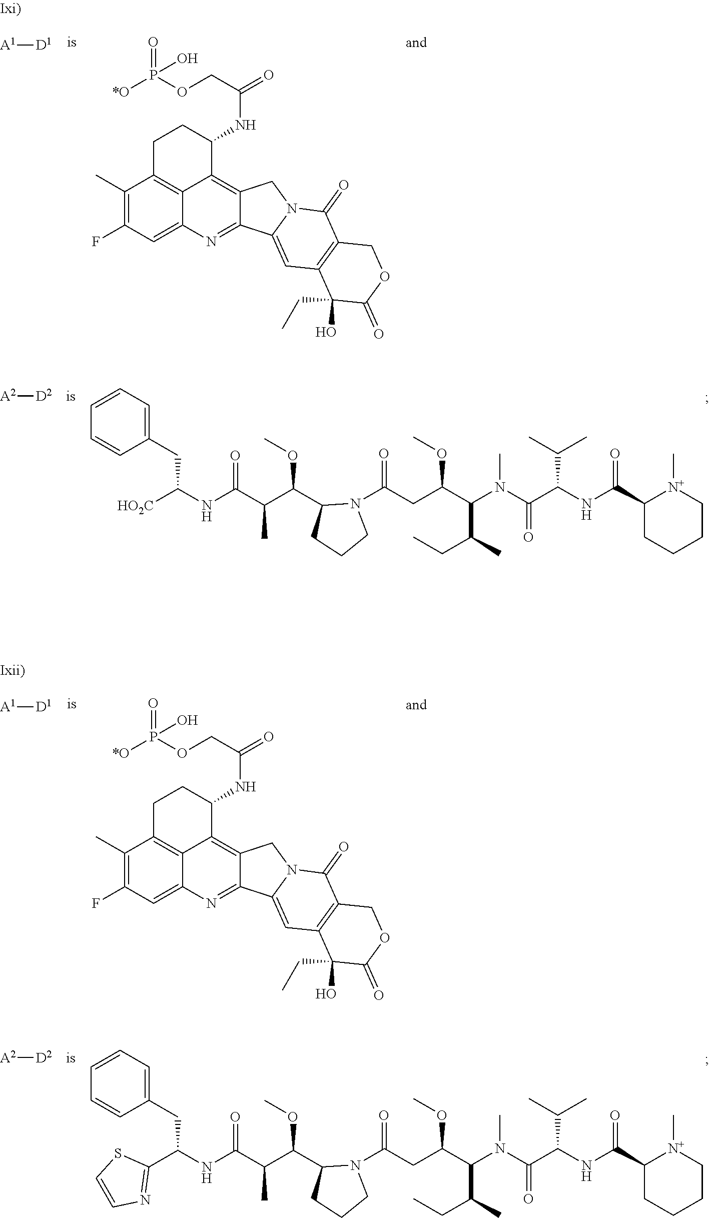

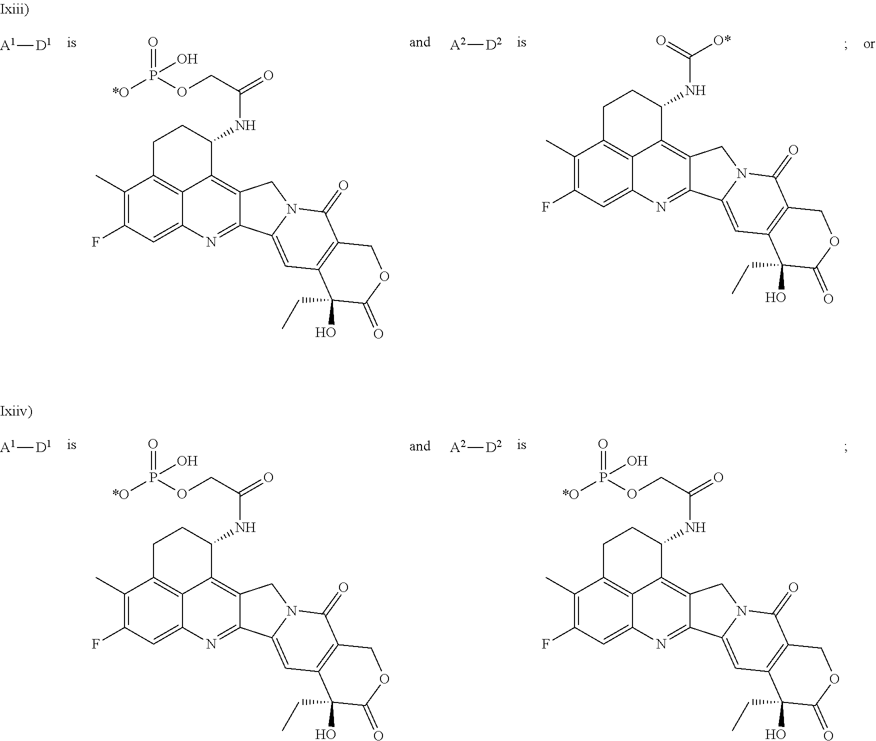

wherein * indicates the point of connection to the remainder of the molecule.
Embodiment 86. The conjugate according to any one of Embodiments 46m to 83, wherein D¹ and/or D² are an auristatin.
Embodiment 86a. The conjugate according to Embodiment 86, wherein both D¹ and D² are an auristatin.
Embodiment 86b. The conjugate according to any one of Embodiments 46m to 83, wherein D¹ and/or D² are a topoisomerase 1 inhibitor.
Embodiment 86c. The conjugate according to Embodiment 86b, wherein both D¹ and D² are a topoisomerase 1 inhibitor.
Embodiment 86d. The conjugate according to any one of Embodiments 46m to 83, wherein one of D¹ and D² is an auristatin and the other of D¹ and D² is a topoisomerase 1 inhibitor.
Embodiment 86e. The conjugate according to Embodiment 86d, wherein D¹ is an auristatin and D² is a topoisomerase 1 inhibitor.
Embodiment 86f. The conjugate according to Embodiment 86d, wherein D¹ is a topoisomerase 1 inhibitor and D² is an auristatin.
Embodiment 87. The conjugate according to Embodiment 86, wherein D¹ and D² are independently selected from the group consisting of

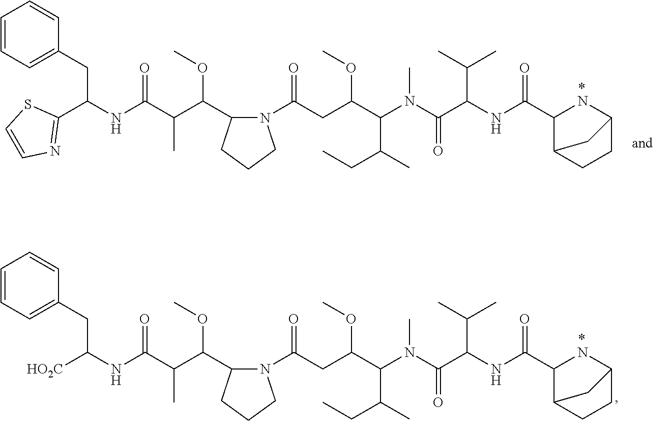

wherein * indicates the point of attachment to A¹ or A².
Embodiment 87a. The conjugate according to Embodiment 87, wherein D¹ and D² are independently selected from the group consisting of

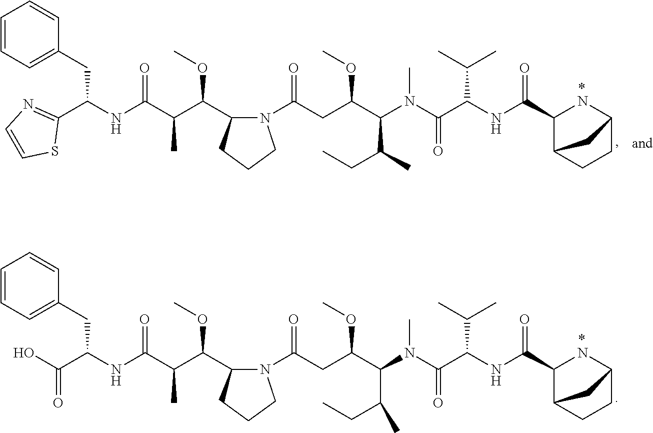

Embodiment 87b. The conjugate according to Embodiment 85a, wherein D¹ and D² are independently selected from the group consisting of:

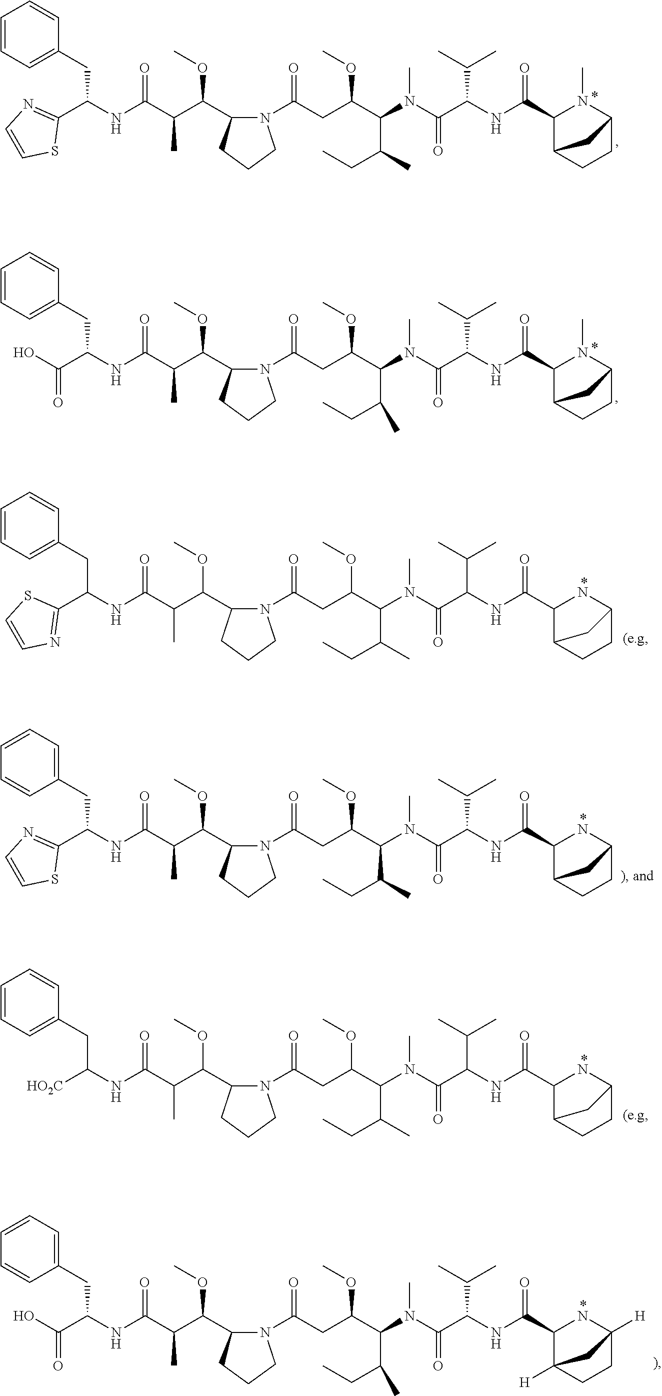

wherein * indicates the point of attachment to A¹ or A²; or a pharmaceutically acceptable salt thereof.
Embodiment 87c. The conjugate according to Embodiment 85a, wherein D¹ and D² are independently selected from the group consisting of:

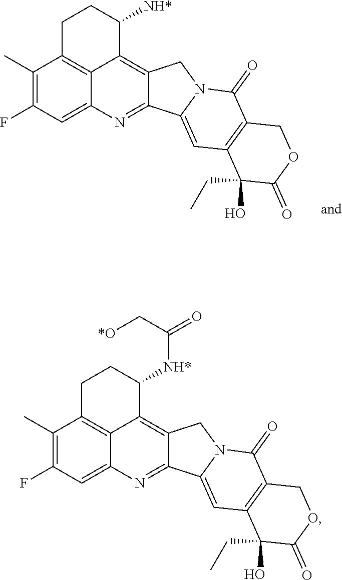

wherein * indicates the point of attachment to A¹ or A²; or a pharmaceutically acceptable salt thereof.
Embodiment 88. The conjugate according to any one of Embodiments 46m to 83, wherein D¹ and D² are each independently selected from an antitubulin agent, a tubulin inhibitor, a DNA minor groove binder, a DNA replication inhibitor, an alkylating agent, an antibiotic, an antifolate, an antimetabolite, a chemotherapy sensitizer, a topoisomerase inhibitor, and/or a vinca alkaloid.
Embodiment 89. The conjugate according to any one of Embodiments 46m to 83, wherein D¹ and D² are each independently selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor, a DHFR inhibitor, or a pro-apoptotic agent, optionally with the proviso that neither D¹ nor D² is a BH3 mimetic.
Embodiment 90. A conjugate comprising an antibody or antigen-binding fragment bonded to one or more (e.g. 1 to 16, e.g. 1 to 4) compounds independently selected from Embodiment 44 or Embodiment 45.
Embodiment 91. The conjugate of any one of Embodiments 46m to 90, wherein the antibody or antigen-binding fragment binds to a target antigen on a cancer cell.
Embodiment 92. The conjugate according to Embodiment 91, wherein:
(i) the target antigen is selected from BCMA, CD33, HER2, CD38, CD48, CD79b, PCAD, CD74, CD138, SLAMF7, CD123, CLL1, FLT3, CD7, CKIT, CD56, SEZ6, DLL3, DLK1, B7-H3, EGFR, CD71, EphA2, EPCAM, FOLR1, ENPP3, MET, AXL, SLC34A2, Nectin4, TROP2, LIV1, CD46, MSLN, F3, MUC16, SLC39A6, TFRC, TACSTD2, and GPNMB;
(ii) the target antigen is selected from EGFR, CD7, HER2, EPCAM, FOLR1, ENPP3, MET, AXL, SLC34A2, Nectin4, MSLN, F3, MUC16, SLC39A6, TFRC, TACSTD2, and GPNMB; or
(iii) the target antigen is selected from PCAD, CD48, CD74, EphA2, TROP2, B7-H3, 5T4, and HER2.
Embodiment 92a. The conjugate of Embodiment 91 or 92, wherein the antibody or antigen-binding fragment thereof is selected from Table D1.
Embodiment 93. The conjugate according to any one of Embodiments 91 to 92a, wherein the antibody or antigen-binding fragment comprises i) three heavy chain CDR sequences and three light chain CDR sequences selected from Tables D3 and D8, ii) a heavy chain variable region sequence and a light chain variable region sequence selected Tables D2 and D8, or iii) a heavy chain sequence and light chain sequence selected from Tables D4, D5, and D7.
Embodiment 94. The conjugate of Embodiment 91, wherein the antibody or antigen-binding fragment is an anti-CD74 antibody comprising three heavy chain CDRs and three light chain CDRs selected from the group consisting of:
   1) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:268, light chain CDR2 (LCDR2) consisting of SEQ ID NO:264, and light chain CDR3 (LCDR3) consisting of SEQ ID NO:265;
   2) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
   3) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:269, light chain CDR2 (LCDR2) consisting of SEQ ID NO:264, and light chain CDR3 (LCDR3) consisting of SEQ ID NO:174;
   4) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174;
   5) heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:263, light chain CDR2 (LCDR2) consisting of SEQ ID NO:264, and light chain CDR3 (LCDR3) consisting of SEQ ID NO:265;
   6) heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174;
   7) heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:215, light chain CDR2 (LCDR2) consisting of SEQ ID NO:264, and light chain CDR3 (LCDR3) consisting of SEQ ID NO:174; and
   8) heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174.
Embodiment 95. The conjugate of Embodiment 91, wherein the antibody or antigen-binding fragment is an anti-CD74 antibody comprising (a) a heavy chain variable region comprising the amino acid sequence of SEQ ID NO:153, and a light chain variable region comprising the amino acid sequence of SEQ ID NO:262, or (b) a heavy chain variable region comprising the amino acid sequence of SEQ ID NO:153, and a light chain variable region comprising the amino acid sequence of SEQ ID NO:267.
Embodiment 96. The conjugate of Embodiment 91, wherein the antibody or antigen-binding fragment is an anti-CD74 antibody comprising:
(a) the heavy chain amino acid sequence of SEQ ID NO: 118 or a sequence that is at least 95% identical to SEQ ID NO:118, and the light chain amino acid sequence of SEQ ID NO:237 or a sequence that is at least 95% identical to SEQ ID NO:237;
(b) the heavy chain amino acid sequence of SEQ ID NO:236 or a sequence that is at least 95% identical to SEQ ID NO:236, and the light chain amino acid sequence of SEQ ID NO:237 or a sequence that is at least 95% identical to SEQ ID NO:237; or
(c) the heavy chain amino acid sequence of SEQ ID NO:118 or a sequence that is at least 95% identical to SEQ ID NO:118, and the light chain amino acid sequence of SEQ ID NO:239 or a sequence that is at least 95% identical to SEQ ID NO:239.
Embodiment 97. The conjugate of Embodiment 91, wherein the antibody or antigen-binding fragment is an anti-CD48 antibody comprising three heavy chain CDRs and three light chain CDRs selected from the group consisting of:
   1) heavy chain CDR1 (HCDR1) consisting of SEQ ID NO:271, heavy chain CDR2 (HCDR2) consisting of SEQ ID NO:272, heavy chain CDR3 (HCDR3) consisting of SEQ ID NO:273; light chain CDR1 (LCDR1) consisting of SEQ ID NO:281, light chain CDR2 (LCDR2) consisting of SEQ ID NO:282, and light chain CDR3 (LCDR3) consisting of SEQ ID NO:283;
   2) heavy chain CDR1 (HCDR1) consisting of SEQ ID NO:274, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:285, and light chain CDR3 (LCDR3) consisting of SEQ ID NO:286;
   3) heavy chain CDR1 (HCDR1) consisting of SEQ ID NO:276, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:287, light chain CDR2 (LCDR2) consisting of SEQ ID NO:282, and light chain CDR3 (LCDR3) consisting of SEQ ID NO:286; and
   4) heavy chain CDR1 (HCDR1) consisting of SEQ ID NO:279, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:288, and light chain CDR3 (LCDR3) consisting of SEQ ID NO:286; and
   5) heavy chain CDR1 (HCDR1) consisting of SEQ ID NO:51, heavy chain CDR2 (HCDR2) consisting of SEQ ID NO:52, heavy chain CDR3 (HCDR3) consisting of SEQ ID NO:53; light chain CDR1 (LCDR1) consisting of SEQ ID NO:54, light chain CDR2 (LCDR2) consisting of SEQ ID NO:55, and light chain CDR3 (LCDR3) consisting of SEQ ID NO:56.
Embodiment 98. The conjugate of Embodiment 91, wherein the antibody or antigen-binding fragment is an anti-CD48 antibody comprising a) a heavy chain variable region comprising the amino acid sequence of SEQ ID NO:270, and a light chain variable region comprising the amino acid sequence of SEQ ID NO:280; or b) a heavy chain variable region comprising the amino acid sequence of SEQ ID NO:13, and a light chain variable region comprising the amino acid sequence of SEQ ID NO:14.
Embodiment 99. The conjugate of Embodiment 91, wherein the antibody or antigen-binding fragment is an anti-CD48 antibody comprising (a) the heavy chain amino acid sequence of SEQ ID NO:240 or a sequence that is at least 95% identical to SEQ ID NO:240, and the light chain amino acid sequence of SEQ ID NO:243 or a sequence that is at least 95% identical to SEQ ID NO:243; or (b) the heavy chain amino acid sequence of SEQ ID NO:242 or a sequence that is at least 95% identical to SEQ ID NO:242, and the light chain amino acid sequence of SEQ ID NO:243 or a sequence that is at least 95% identical to SEQ ID NO:243; c) the heavy chain amino acid sequence of SEQ ID NO:240 or a sequence that is at least 95% identical to SEQ ID NO:240, and the light chain amino acid sequence of SEQ ID NO:69 or a sequence that is at least 95% identical to SEQ ID NO:70.
Embodiment 100. The conjugate of Embodiment 91, wherein the antibody or antigen-binding fragment is an anti-Her2 antibody comprising three heavy chain CDRs and three light chain CDRs selected from the group consisting of:
   1) heavy chain CDR1 (HCDR1) consisting of SEQ ID NO:289, heavy chain CDR2 (HCDR2) consisting of SEQ ID NO:290, heavy chain CDR3 (HCDR3) consisting of SEQ ID NO:291; light chain CDR1 (LCDR1) consisting of SEQ ID NO:297, light chain CDR2 (LCDR2) consisting of SEQ ID NO:298, and light chain CDR3 (LCDR3) consisting of SEQ ID NO:299;
   2) heavy chain CDR1 (HCDR1) consisting of SEQ ID NO:292, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44;
   3) heavy chain CDR1 (HCDR1) consisting of SEQ ID NO:293, heavy chain CDR2 (HCDR2) consisting of SEQ ID NO:294, heavy chain CDR3 (HCDR3) consisting of SEQ ID NO:295; light chain CDR1 (LCDR1) consisting of SEQ ID NO:302, light chain CDR2 (LCDR2) consisting of SEQ ID NO:298, and light chain CDR3 (LCDR3) consisting of SEQ ID NO:44; and
   4) heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44.
Embodiment 101. The conjugate of Embodiment 91, wherein the antibody or antigen-binding fragment is an anti-Her2 antibody comprising a heavy chain variable region comprising the amino acid sequence of SEQ ID NO:9, and a light chain variable region comprising the amino acid sequence of SEQ ID NO:296.
Embodiment 102. The conjugate of Embodiment 91, wherein the antibody or antigen-binding fragment is an anti-Her2 antibody comprising the heavy chain amino acid sequence of SEQ ID NO:245 or a sequence that is at least 95% identical to SEQ ID NO:245, and the light chain amino acid sequence of SEQ ID NO:66 or a sequence that is at least 95% identical to SEQ ID NO:66.
Embodiment 103. The conjugate of Embodiment 91, wherein the antibody or antigen-binding fragment is an anti-PCAD antibody comprising three heavy chain CDRs and three light chain CDRs selected from the group consisting of:
   1) heavy chain CDR1 (HCDR1) consisting of SEQ ID NO:304, heavy chain CDR2 (HCDR2) consisting of SEQ ID NO:305, heavy chain CDR3 (HCDR3) consisting of SEQ ID NO:306; light chain CDR1 (LCDR1) consisting of SEQ ID NO:312, light chain CDR2 (LCDR2) consisting of SEQ ID NO:313, and light chain CDR3 (LCDR3) consisting of SEQ ID NO:314;
   2) heavy chain CDR1 (HCDR1) consisting of SEQ ID NO:307, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316;
   3) heavy chain CDR1 (HCDR1) consisting of SEQ ID NO:309, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:317, light chain CDR2 (LCDR2) consisting of SEQ ID NO:313, and light chain CDR3 (LCDR3) consisting of SEQ ID NO:316; and
   4) heavy chain CDR1 (HCDR1) consisting of SEQ ID NO:310, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316.
Embodiment 104. The conjugate of Embodiment 91, wherein the antibody or antigen-binding fragment is an anti-PCAD antibody comprising a heavy chain variable region comprising the amino acid sequence of SEQ ID NO:303, and a light chain variable region comprising the amino acid sequence of SEQ ID NO:311.
Embodiment 105. The conjugate of Embodiment 91, wherein the antibody or antigen-binding fragment is an anti-PCAD antibody comprising the heavy chain amino acid sequence of SEQ ID NO:248 or a sequence that is at least 95% identical to SEQ ID NO:248, and the light chain amino acid sequence of SEQ ID NO:250 or a sequence that is at least 95% identical to SEQ ID NO:250.
Embodiment 106. The conjugate of Embodiment 91, wherein the antibody or antigen-binding fragment is an anti-EphA2 antibody comprising three heavy chain CDRs and three light chain CDRs selected from the group consisting of:
   1) heavy chain CDR1 (HCDR1) consisting of SEQ ID NO:319, heavy chain CDR2 (HCDR2) consisting of SEQ ID NO:320, heavy chain CDR3 (HCDR3) consisting of SEQ ID NO:321; light chain CDR1 (LCDR1) consisting of SEQ ID NO:330, light chain CDR2 (LCDR2) consisting of SEQ ID NO:331, and light chain CDR3 (LCDR3) consisting of SEQ ID NO:332;
   2) heavy chain CDR1 (HCDR1) consisting of SEQ ID NO:322, heavy chain CDR2 (HCDR2) consisting of SEQ ID NO:323, heavy chain CDR3 (HCDR3) consisting of SEQ ID NO:324; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335;
   3) heavy chain CDR1 (HCDR1) consisting of SEQ ID NO:325, heavy chain CDR2 (HCDR2) consisting of SEQ ID NO:326, heavy chain CDR3 (HCDR3) consisting of SEQ ID NO:327; light chain CDR1 (LCDR1) consisting of SEQ ID NO:336, light chain CDR2 (LCDR2) consisting of SEQ ID NO:331, and light chain CDR3 (LCDR3) consisting of SEQ ID NO:335; and
   4) heavy chain CDR1 (HCDR1) consisting of SEQ ID NO:328, heavy chain CDR2 (HCDR2) consisting of SEQ ID NO:323, heavy chain CDR3 (HCDR3) consisting of SEQ ID NO:321; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.
Embodiment 107. The conjugate of Embodiment 91, wherein the antibody or antigen-binding fragment is an anti-EphA2 antibody comprising a heavy chain variable region comprising the amino acid sequence of SEQ ID NO:318, and a light chain variable region comprising the amino acid sequence of SEQ ID NO:329.
Embodiment 108. The conjugate of Embodiment 91, wherein the antibody or antigen-binding fragment is an anti-EphA2 antibody comprising the heavy chain amino acid sequence of SEQ ID NO:252 or a sequence that is at least 95% identical to SEQ ID NO:252, and the light chain amino acid sequence of SEQ ID NO:254 or a sequence that is at least 95% identical to SEQ ID NO:254.
Embodiment 109. The conjugate of any one of Embodiments 94 to 108, wherein the antibody or antigen binding fragment thereof comprises one or more cysteine substitutions selected from E152C, S375C, or both E152C and S375C of the heavy chain of the antibody or antigen binding fragment thereof, wherein the position is numbered according to the EU system.
Embodiment 110. The conjugate of any one of Embodiments 94 to 108, wherein the antibody or antigen binding fragment thereof comprises one or more Fc silencing mutations.
Embodiment 111. A composition comprising multiple copies of the conjugate of any one of Embodiments 46m to 110, wherein the average a of the conjugates in the composition is from about 1 to about 8, e.g., about 1 to about 6, about 1 to about 4, or about 1 to about 2.
Embodiment 112. The compound or pharmaceutically acceptable salt thereof according to any one of Embodiments 1 to 43, represented by any one of the following formulae:

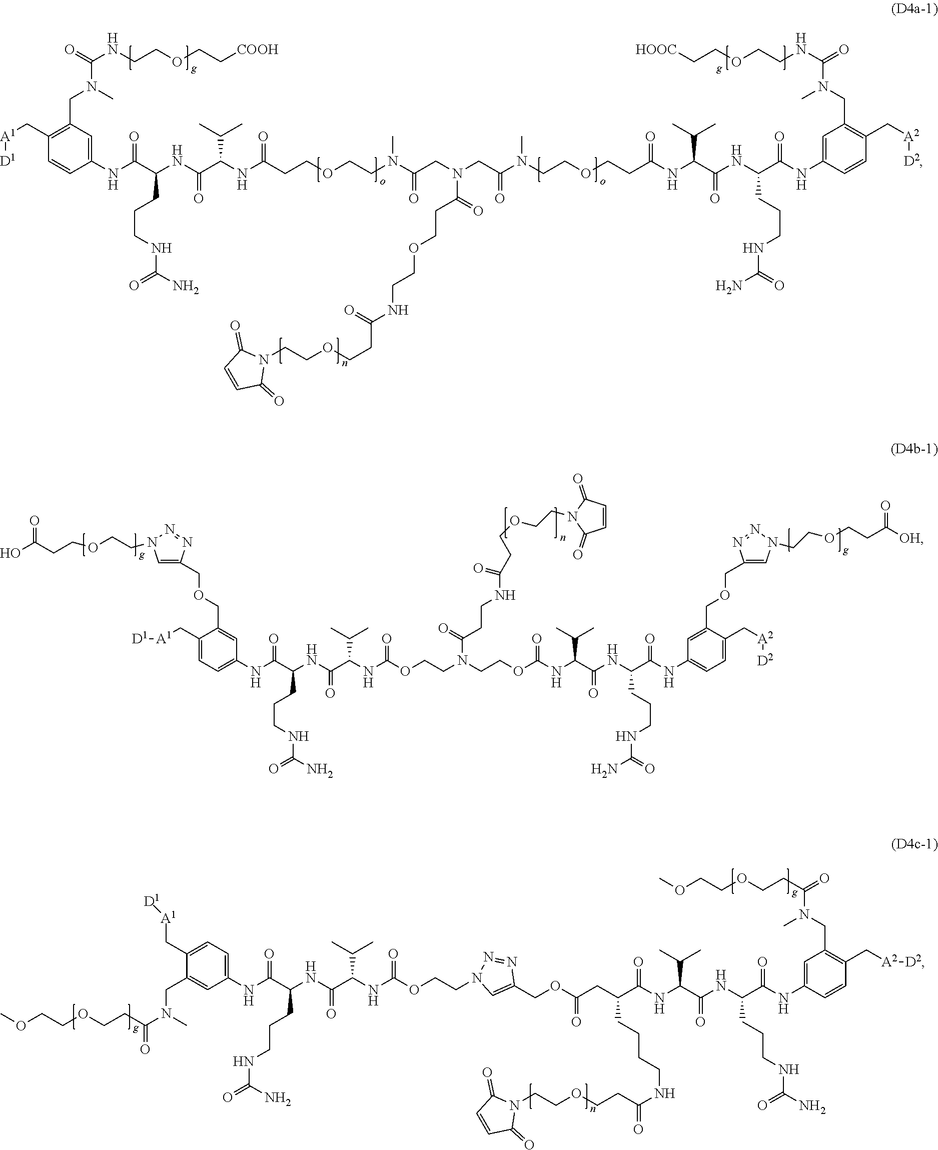

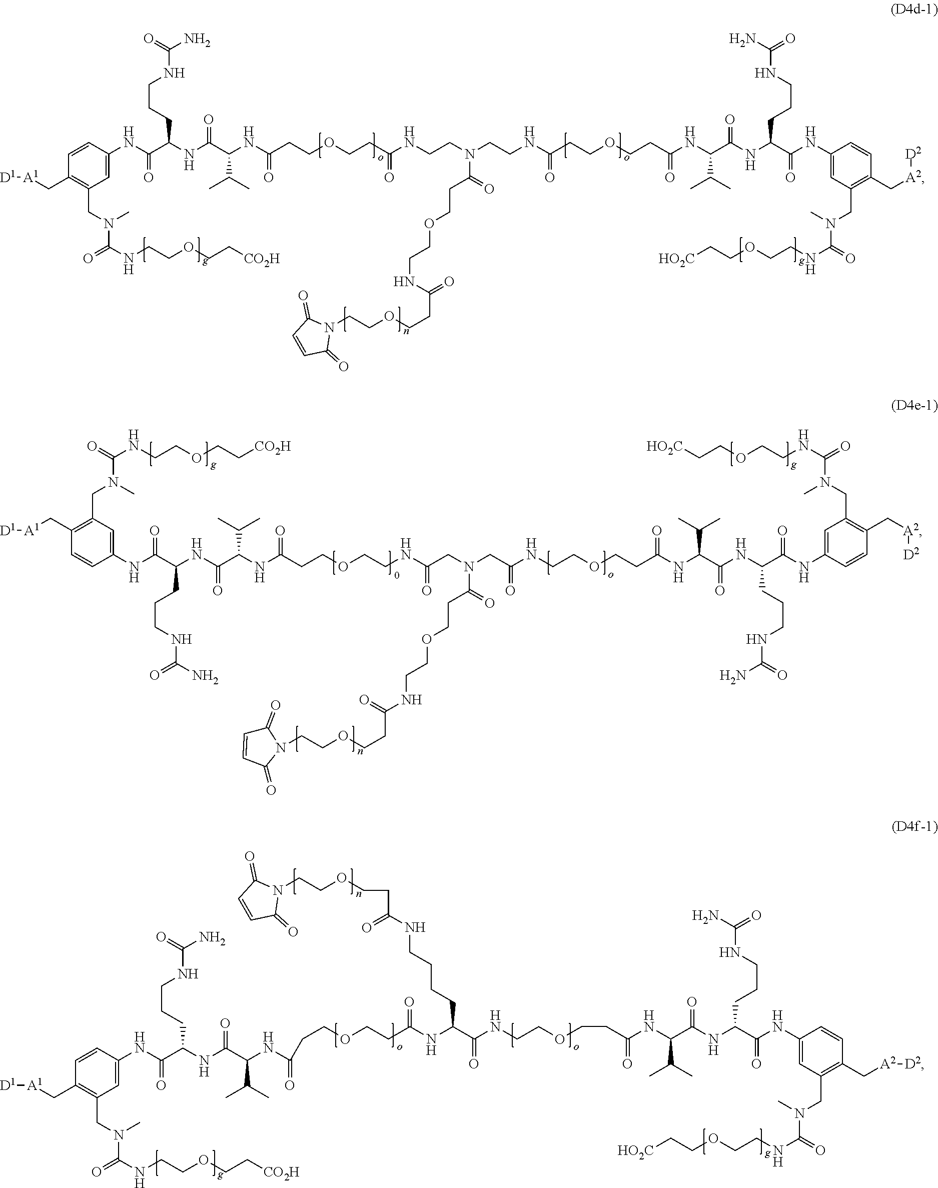

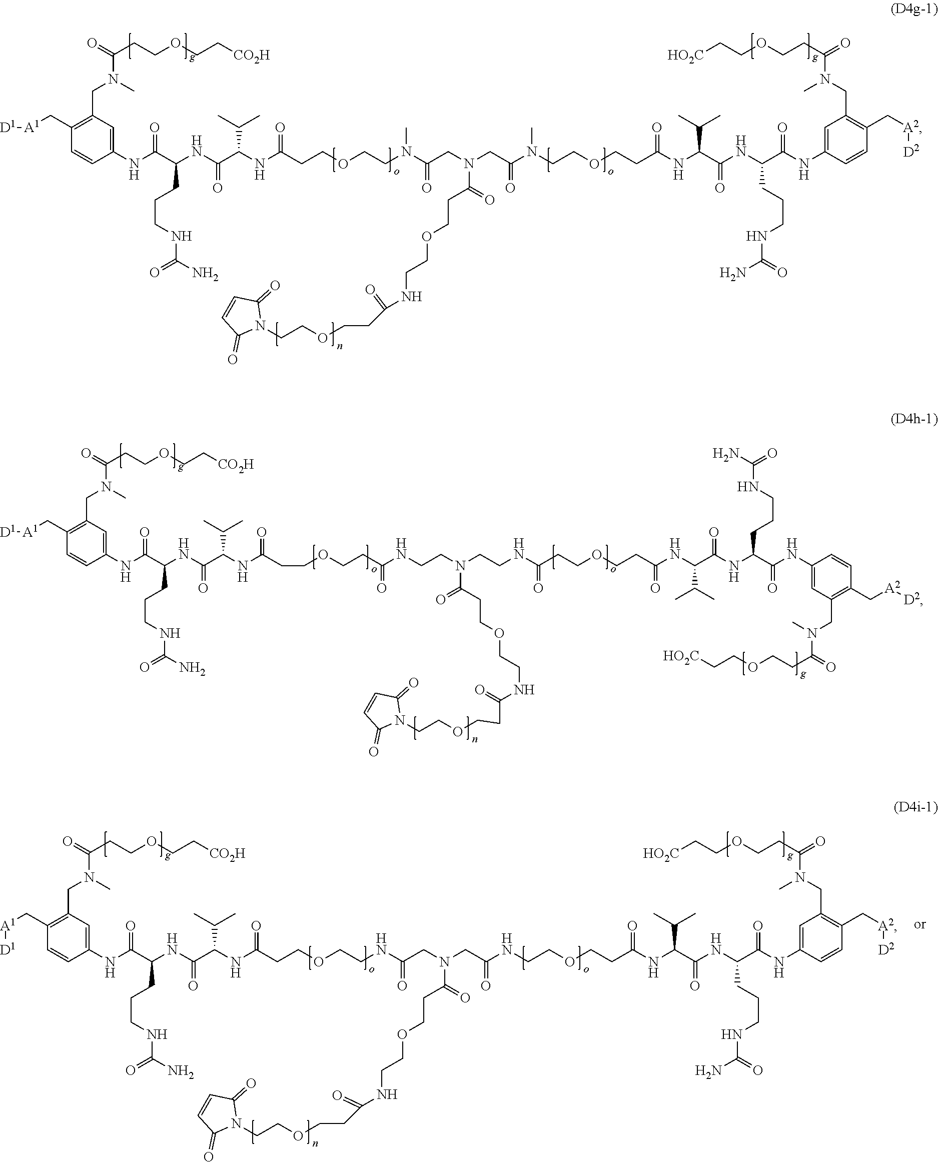

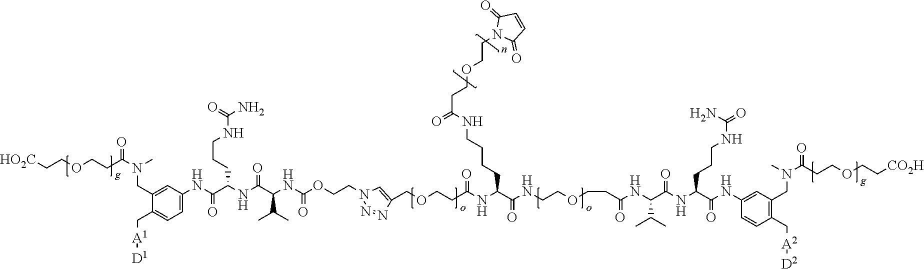

wherein:
   A¹ and A² are each independently selected from a bond,

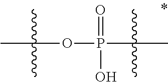 [SMILES: *OP(*)(=O)O]

and —O—C(═O)—*, wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 2 and 5); and
n is an integer between 1 and 12 (e.g., between 2 and 5).
Embodiment 112a. The compound or pharmaceutically acceptable salt thereof according to Embodiment 112, wherein A¹ and A² are each independently selected from a bond and O—C(═O)—*, wherein * in A¹ and A² indicates the point of attachment to D¹ or D².
Embodiment 113. The conjugate according to any one of Embodiments 46m to 110, represented by any one of the following formulae:

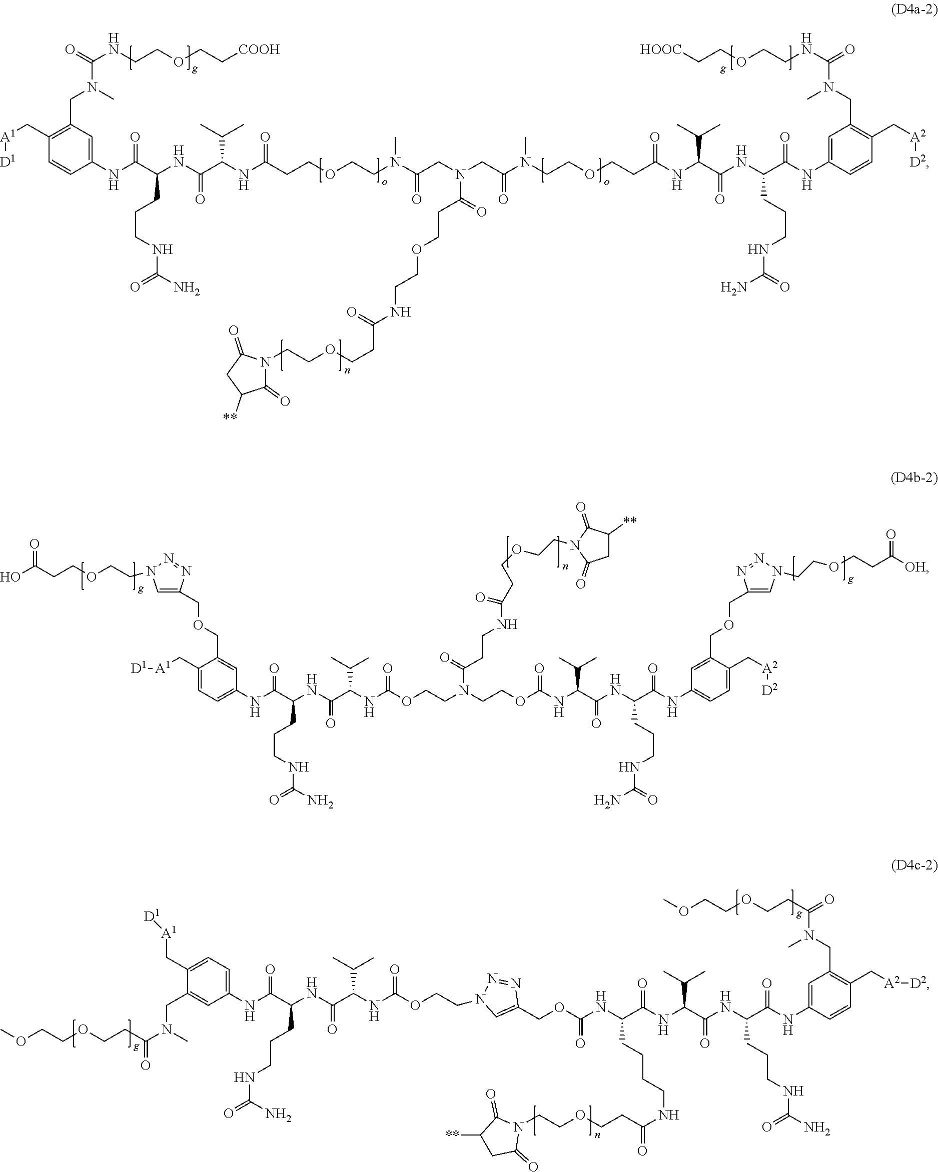

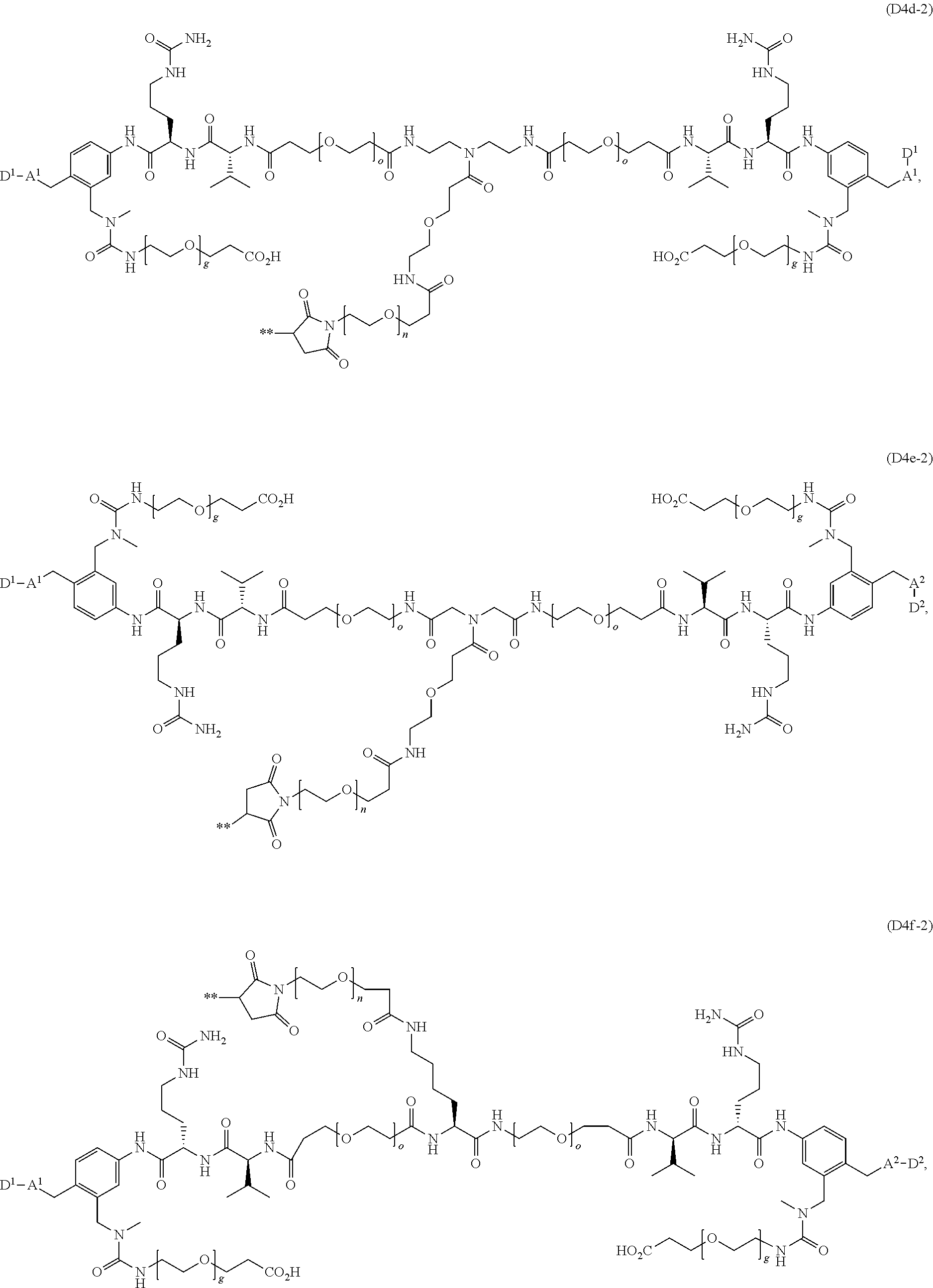

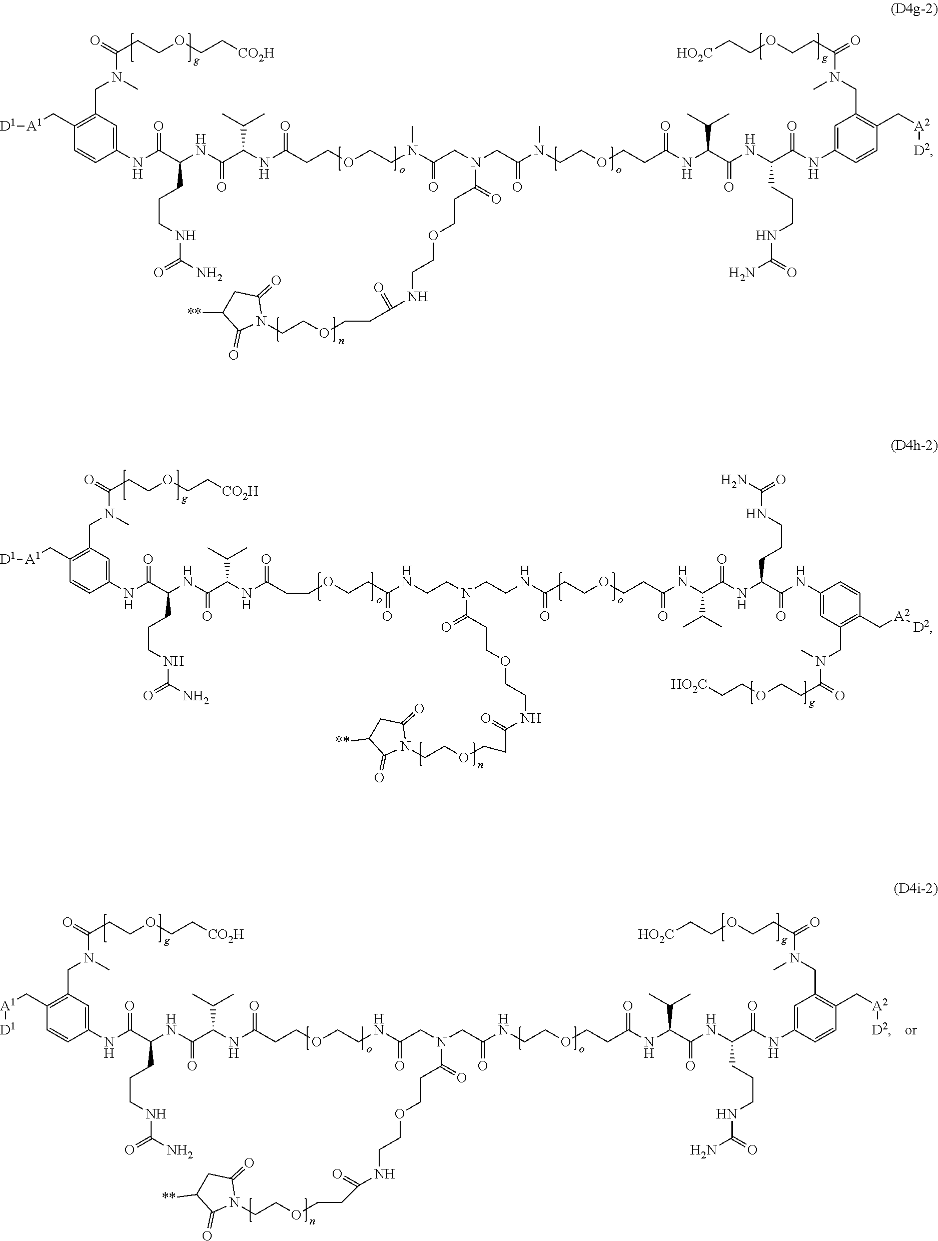

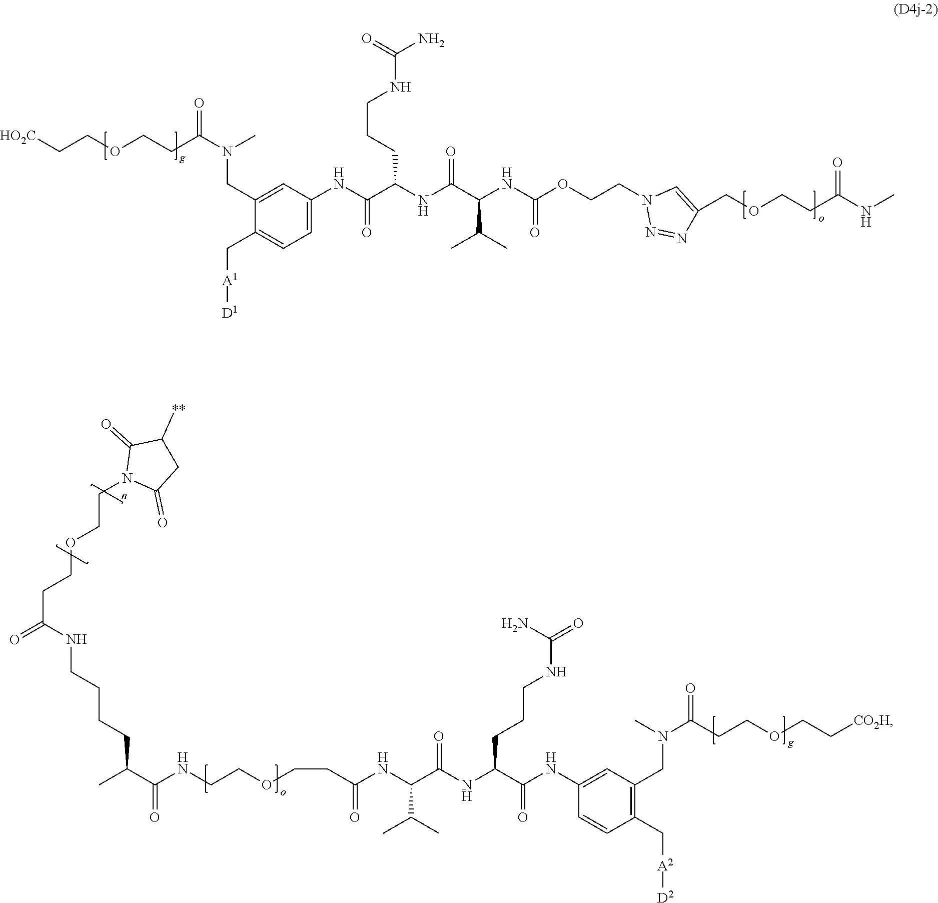

A¹ and A² are each independently selected from a bond,

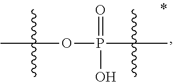 [SMILES: *OP(*)(=O)O]

and —O—C(═O)—*, wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 2 and 5);
n is an integer between 1 and 12 (e.g., between 2 and 5); and
[symbol] indicates the point of attachment to the Ab.
Embodiment 113a. The conjugate according to Embodiment 113, wherein A¹ and A² are each independently selected from a bond and —O—C(═O)—*, wherein * in A¹ and A² indicates the point of attachment to D¹ or D².
Embodiment 114. The compound or pharmaceutically acceptable salt thereof according to any one of Embodiments 1 to 43, of formula (D5a-1):

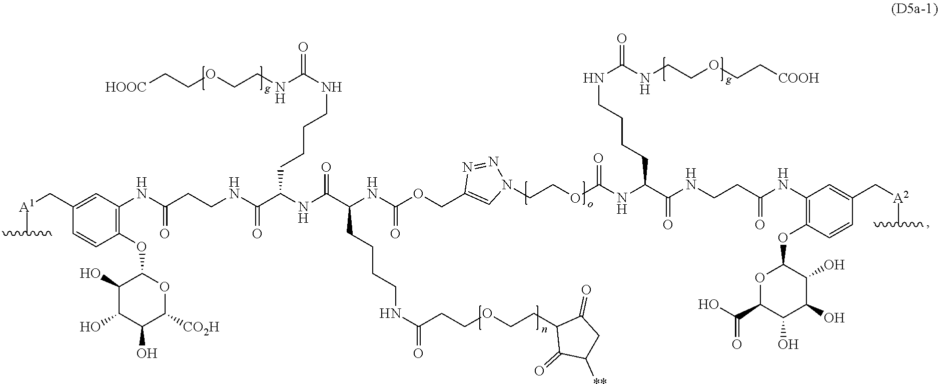

wherein:
   A¹ and A² are each independently selected from a bond,

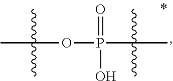 [SMILES: *OP(*)(=O)O]

and —O—C(═O)—*, wherein * in A¹ and A² indicates the point of attachment to D¹ or D²;
   g for each occurrence is independently an integer between 20 and 30 (e.g., 20, 21, 22, 23, 24, 25, 26, 27, 28, 29 or 30);
   o for each occurrence is independently an integer between 1 and 9 (e.g., between 1 and 3); and
   n is an integer between 1 and 12 (e.g., between 5 and 10).
Embodiment 114a. The compound or pharmaceutically acceptable salt thereof according to Embodiment 114, wherein A¹ and A² are each independently selected from a bond and —O—C(═O)—*, wherein * in A¹ and A² indicates the point of attachment to D¹ or D².
Embodiment 115. The conjugate according to any one of Embodiments 46m to 110, of formula (D5a-2):

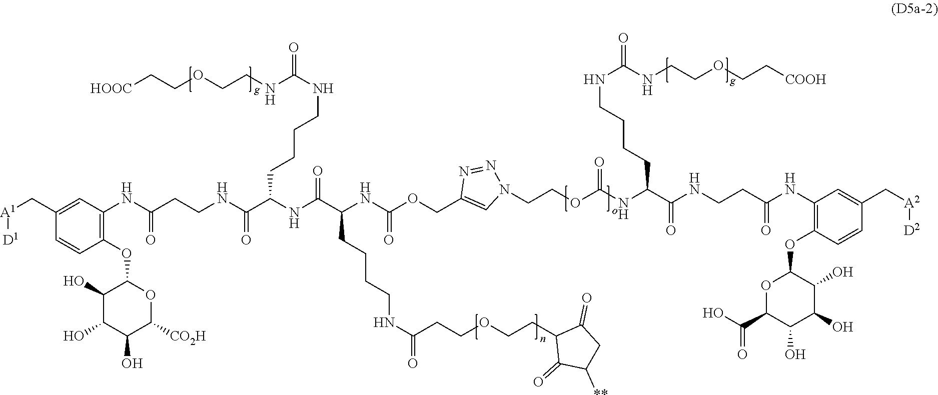

wherein:
   A¹ and A² are each independently selected from a bond,

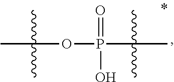 [SMILES: *OP(*)(=O)O]

and —O—C(═O)—*, wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 1 and 3);
n is an integer between 1 and 12 (e.g., between 5 and 10); and
[symbol] indicates the point of attachment to the Ab.
Embodiment 115a. The conjugate according to Embodiment 115, wherein A¹ and A² are each independently selected from a bond and —O—C(═O)—*, wherein * in A¹ and A² indicates the point of attachment to D¹ or D².
Embodiment 116. The conjugate according to any one of Embodiments 46m to 110, represented by the following formulae:

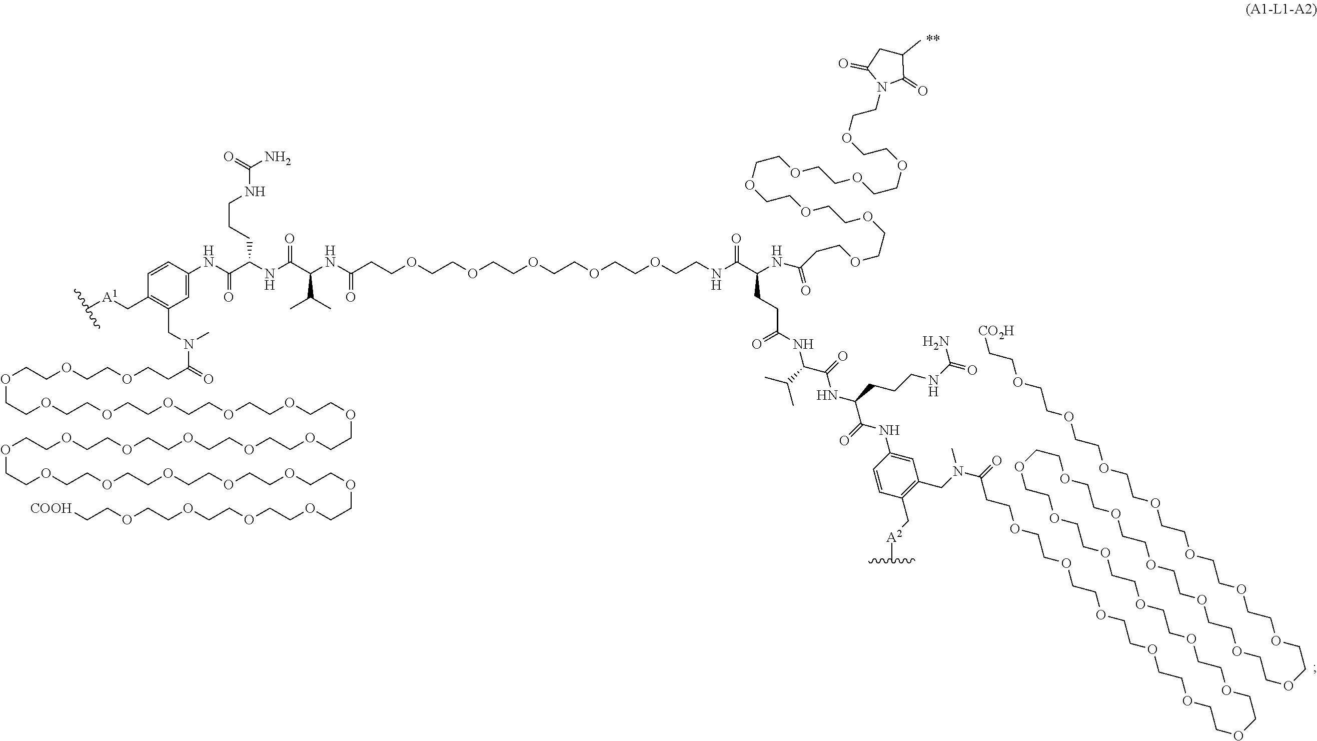

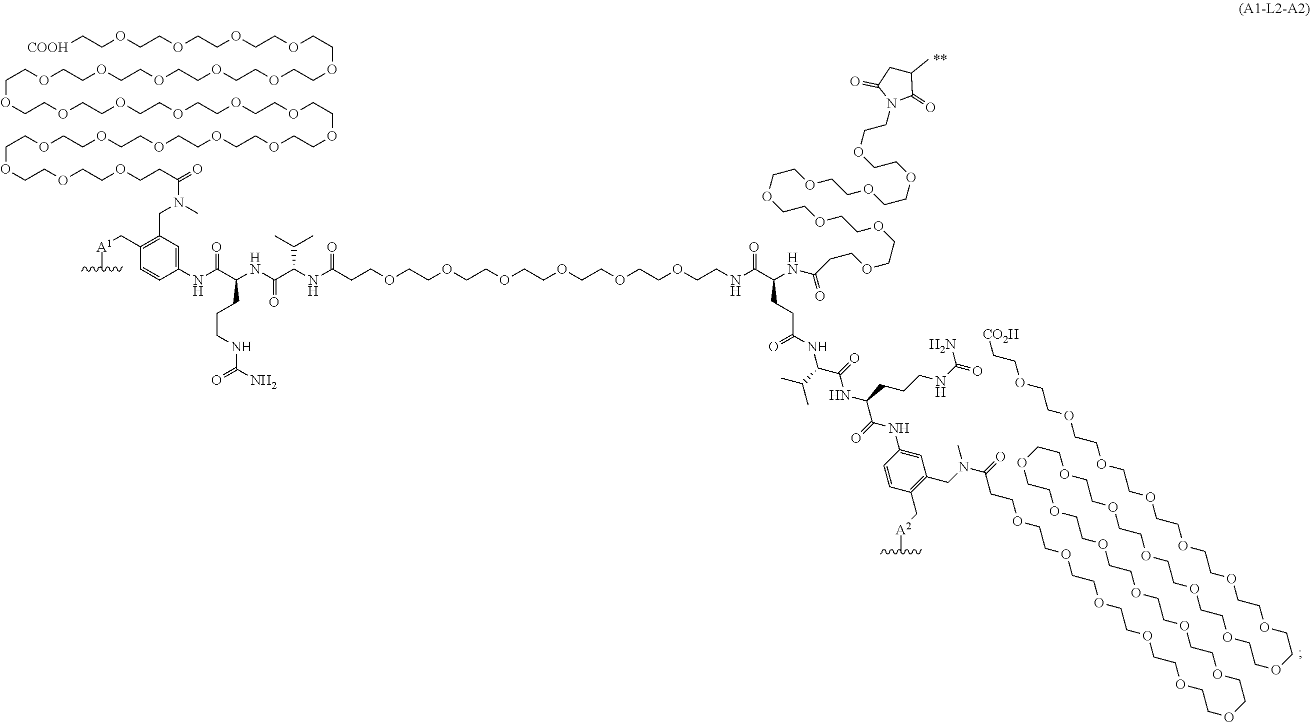

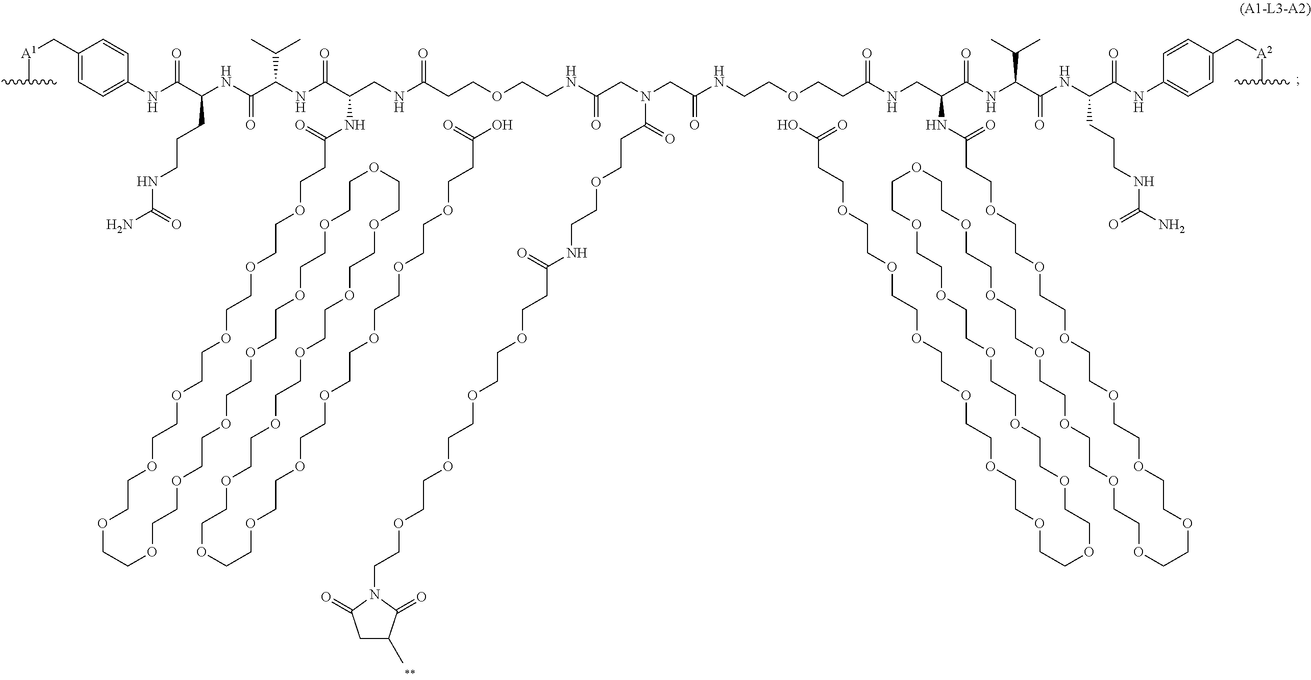

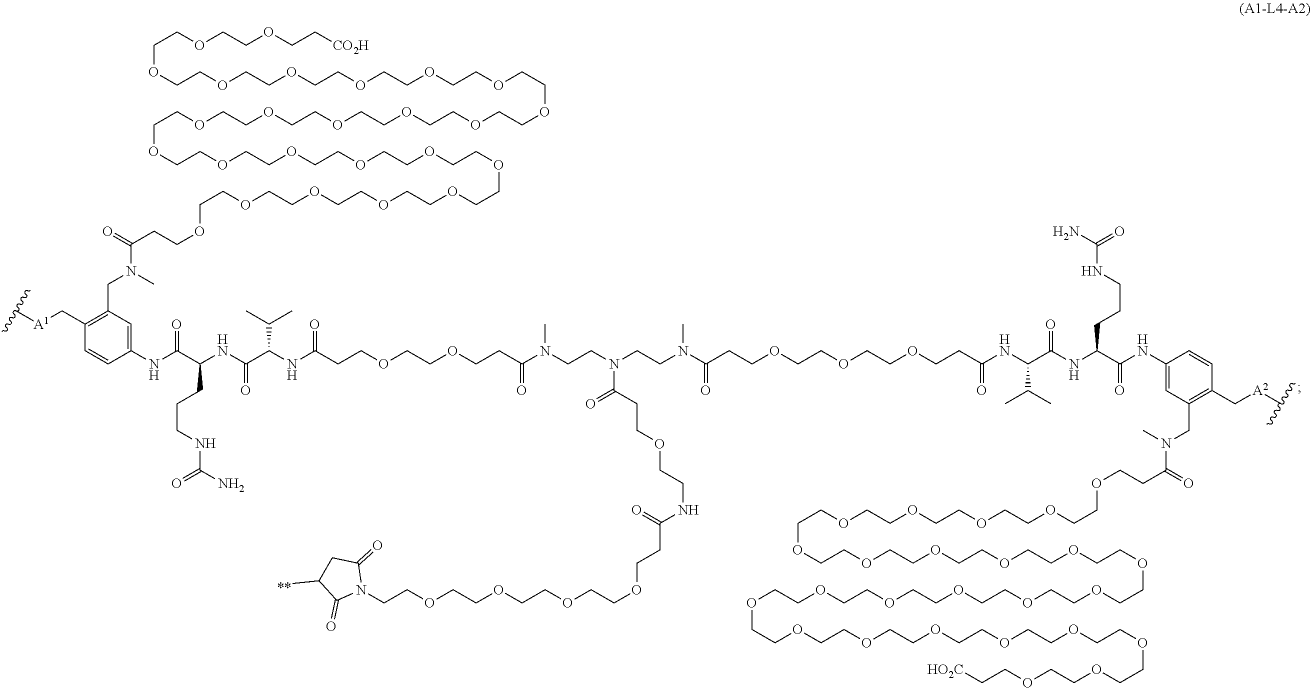

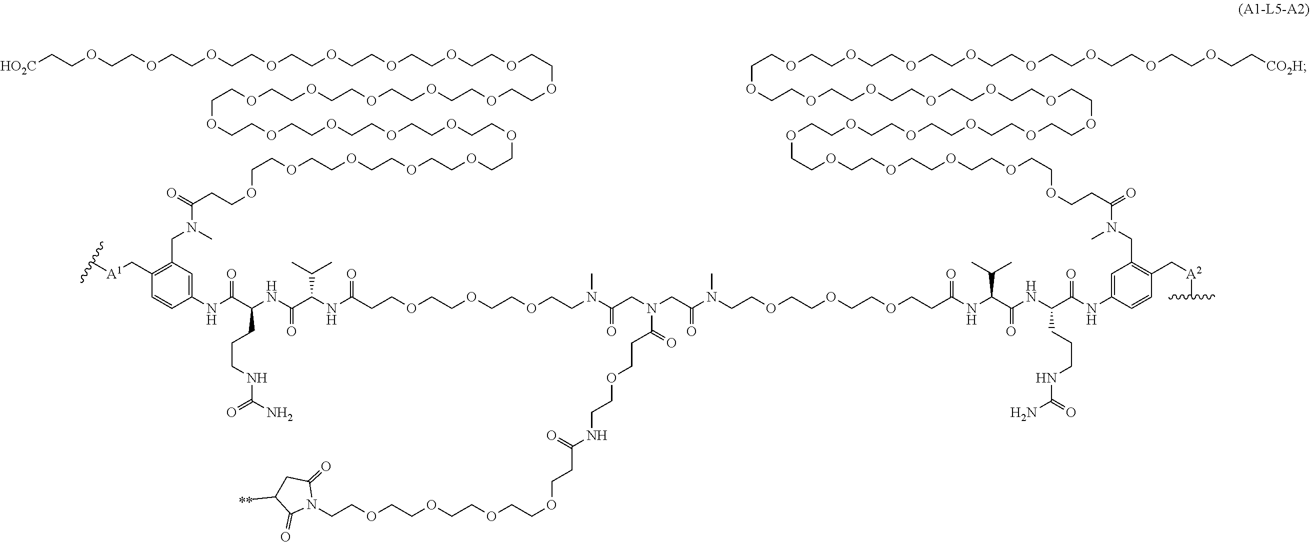

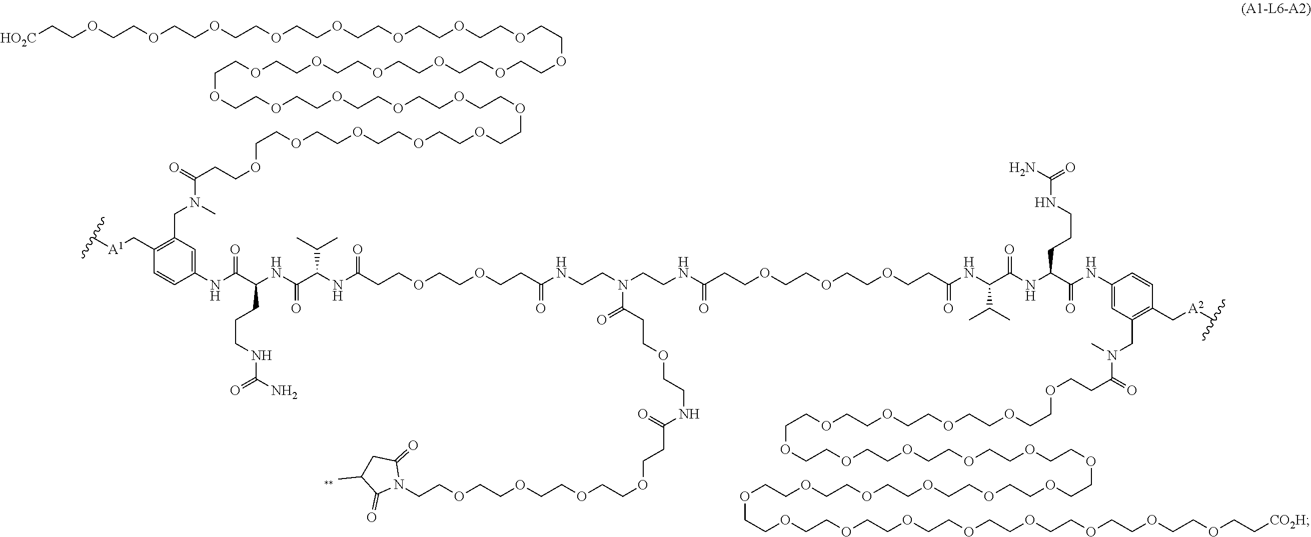

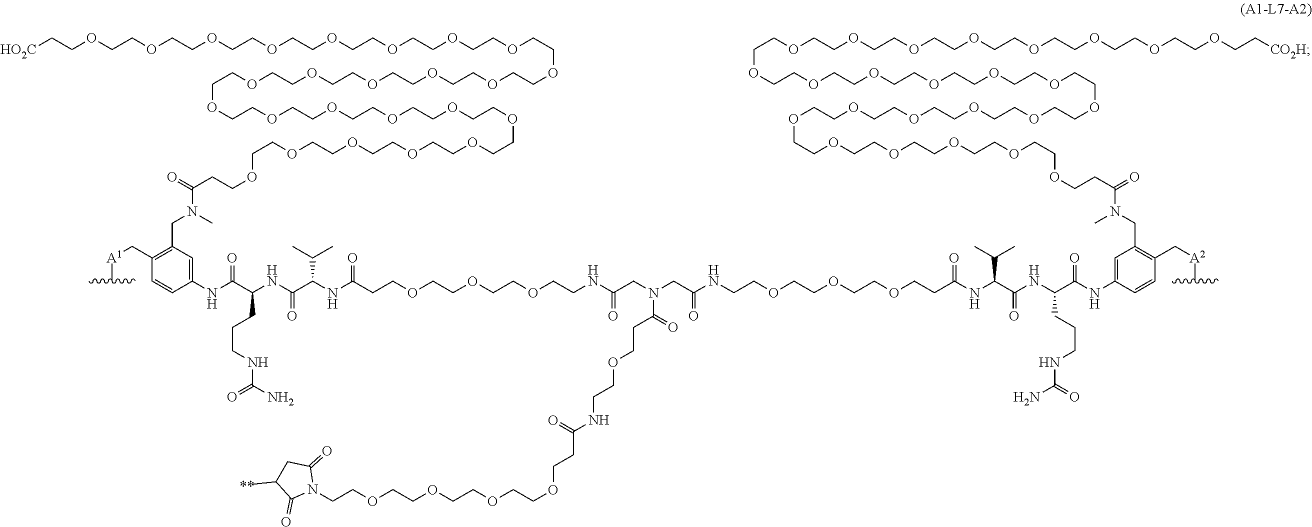

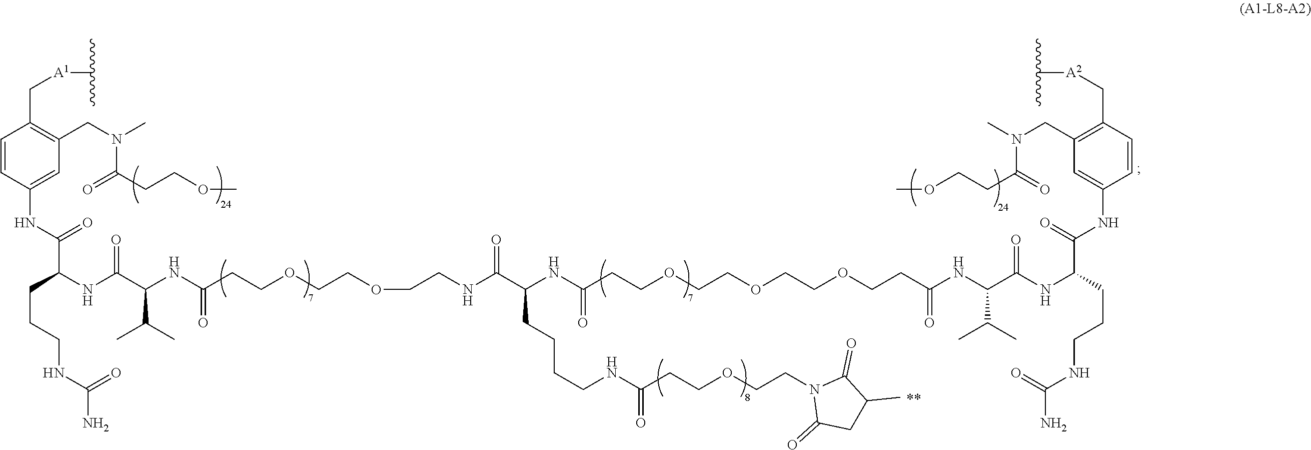

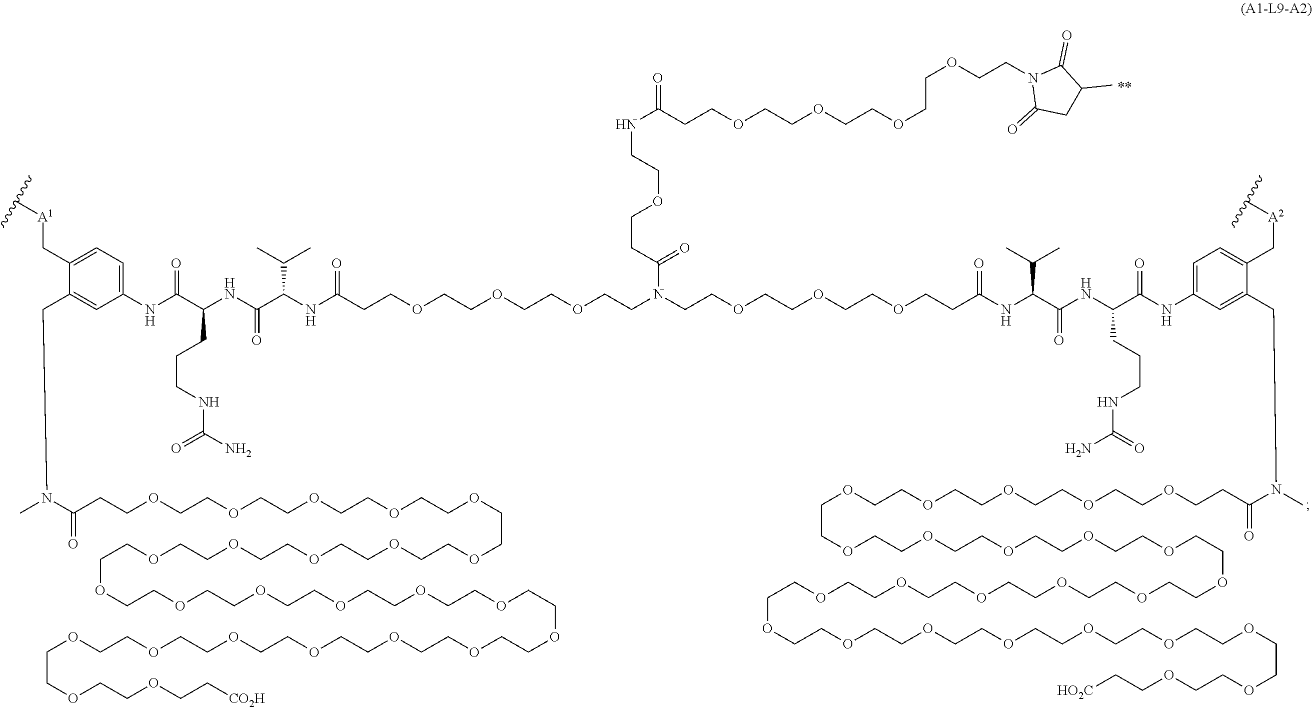

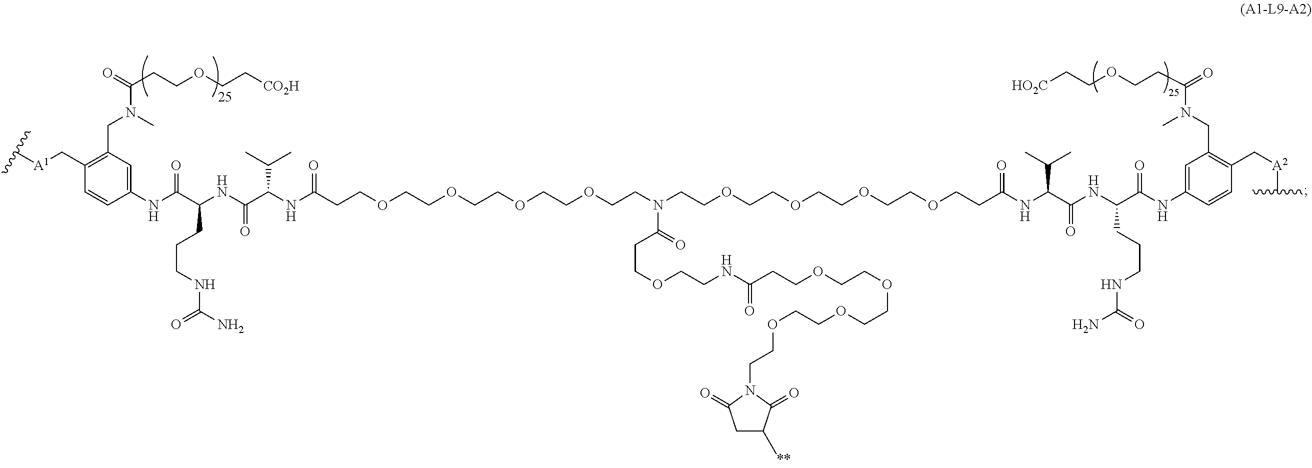

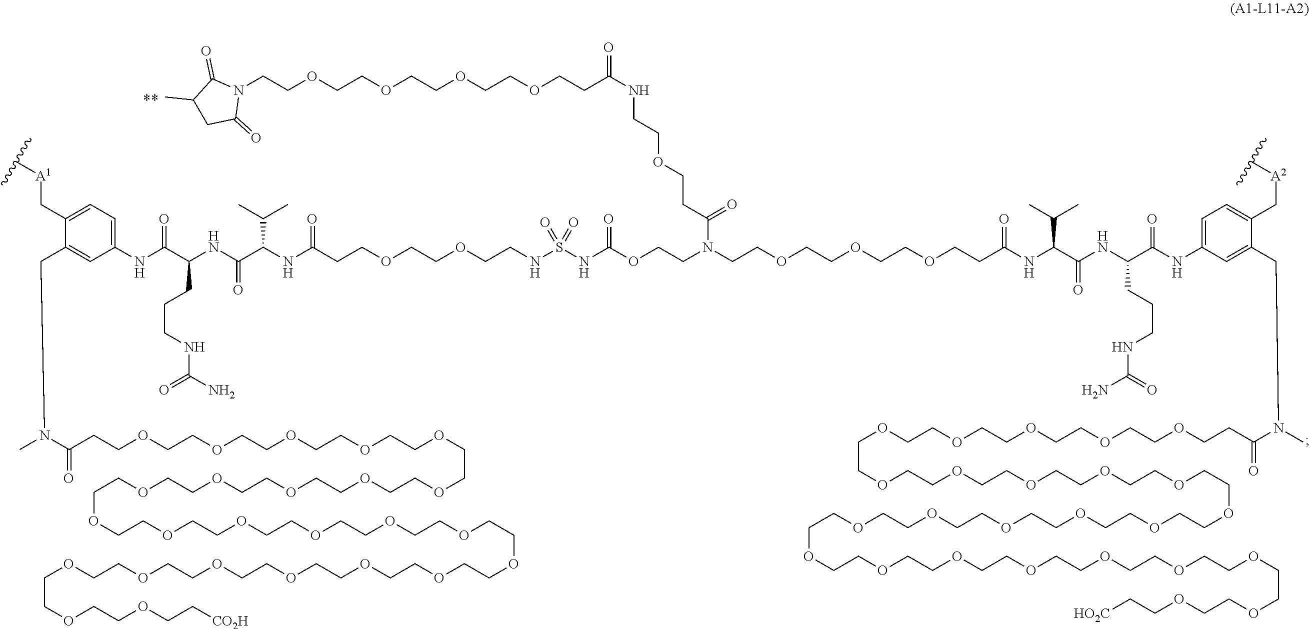

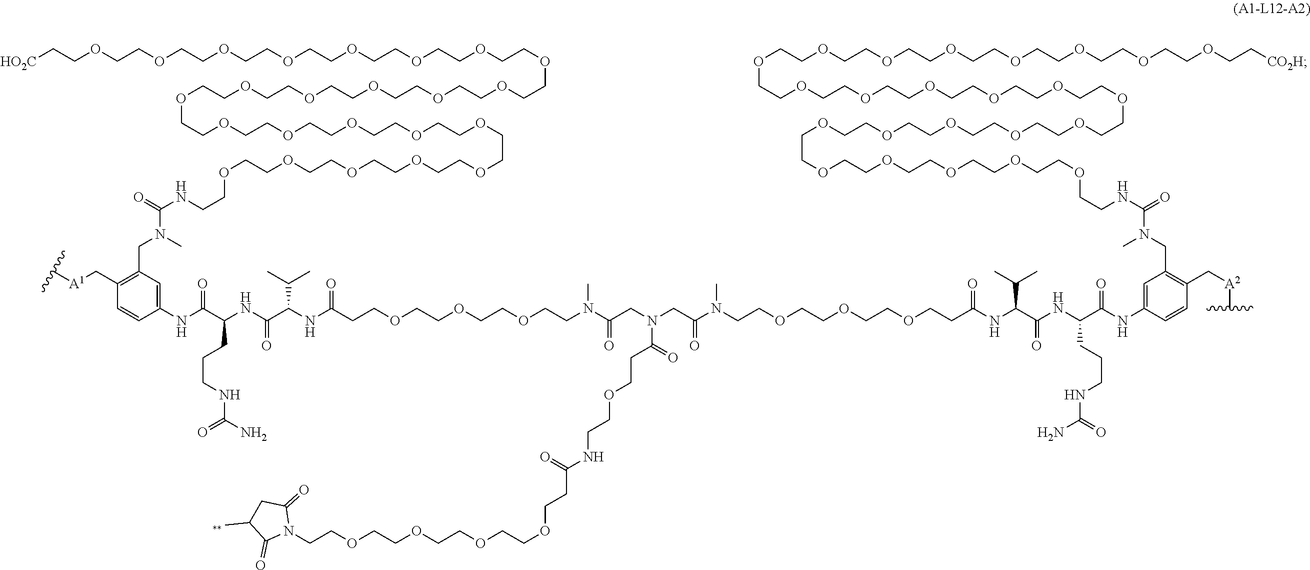

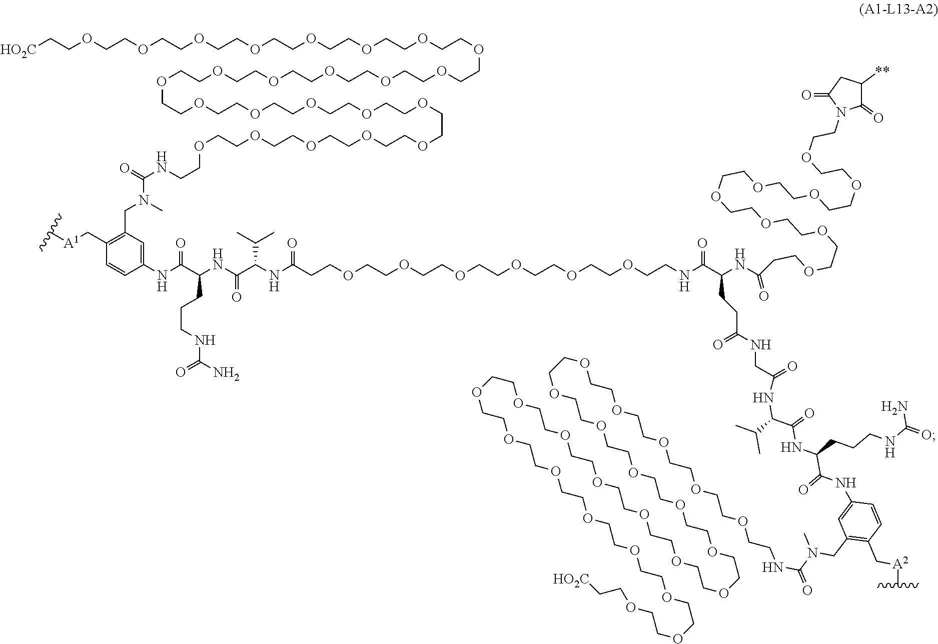

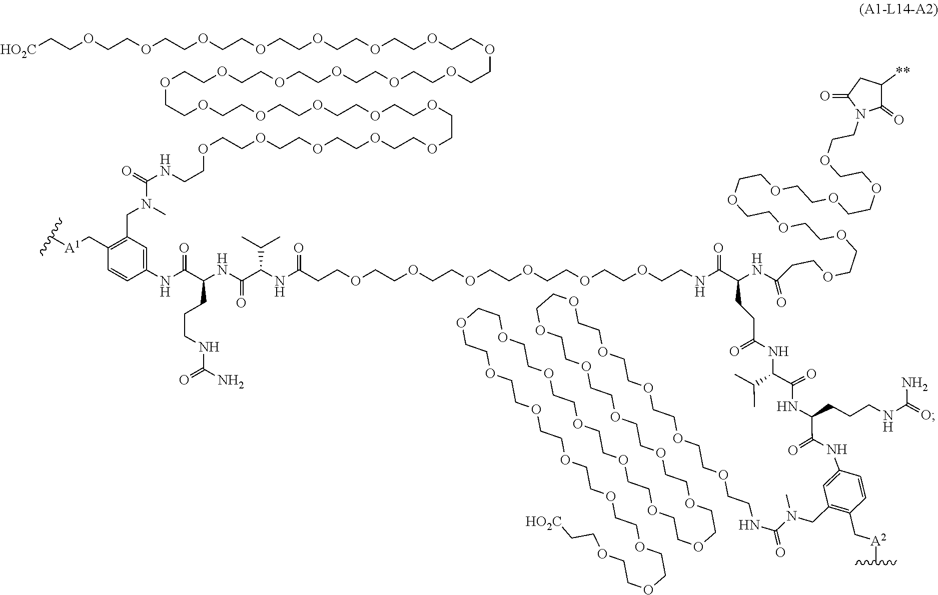

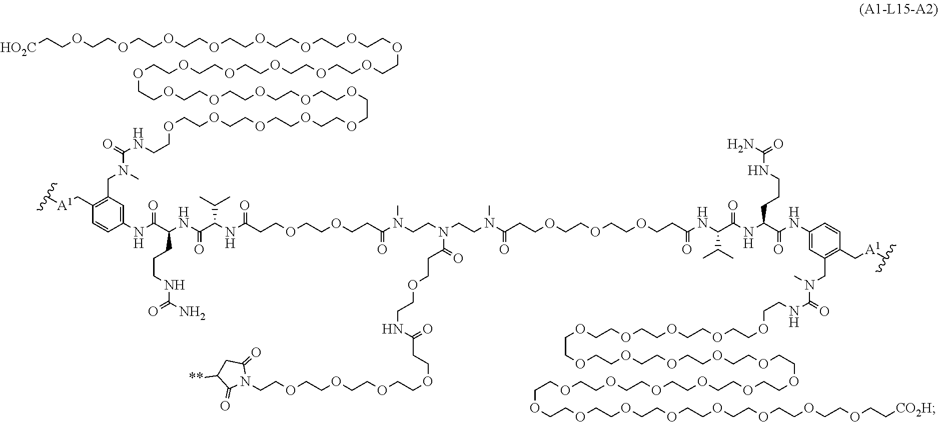

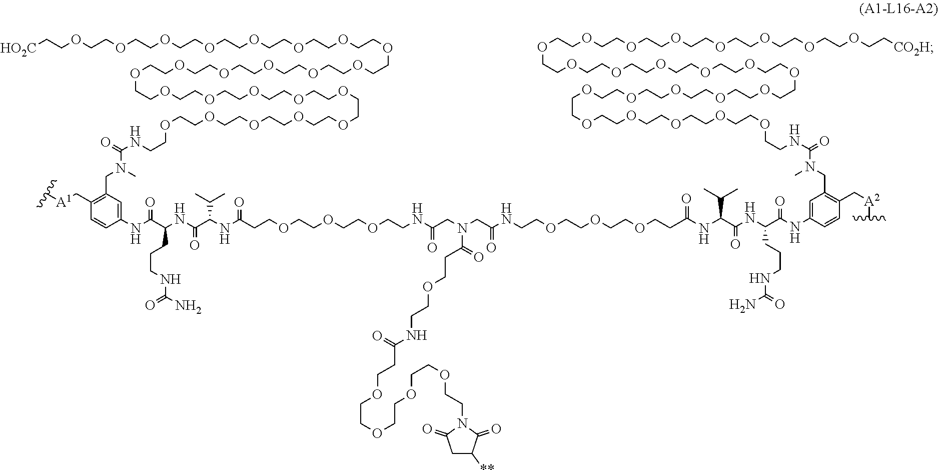

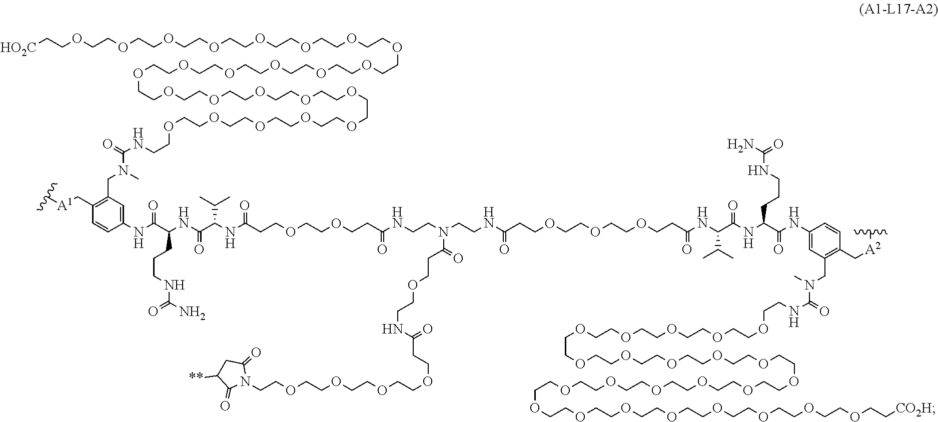

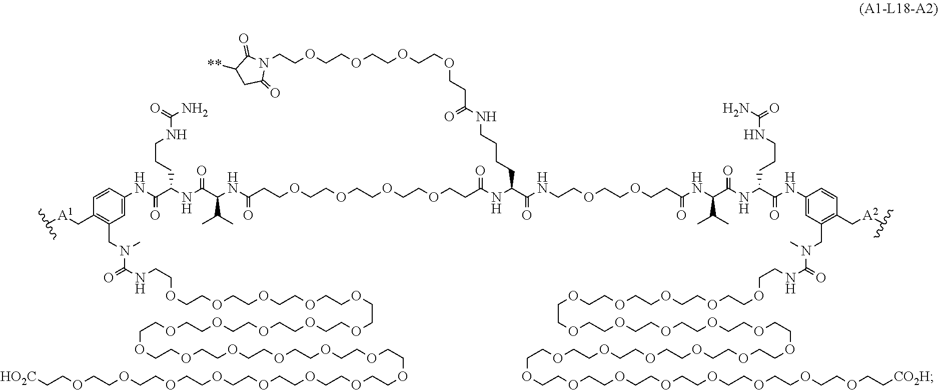

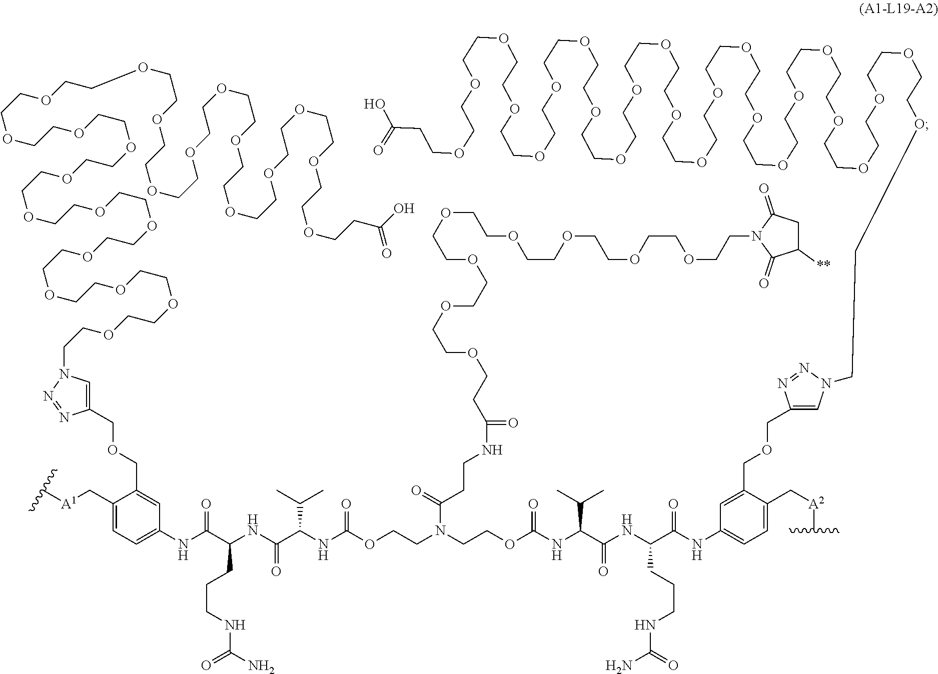

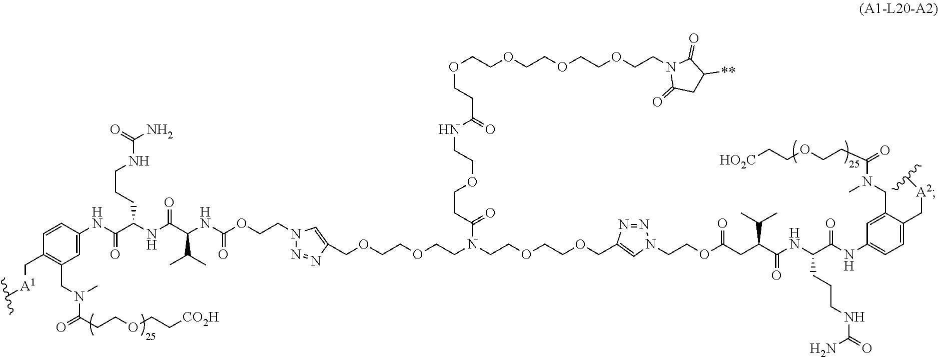

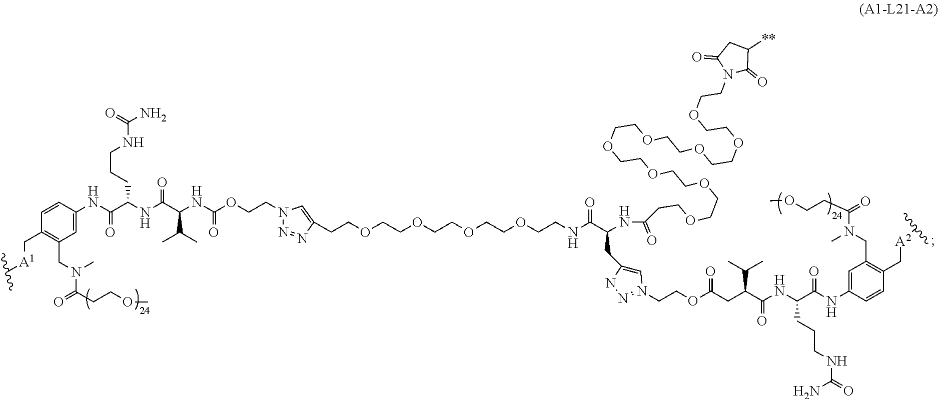

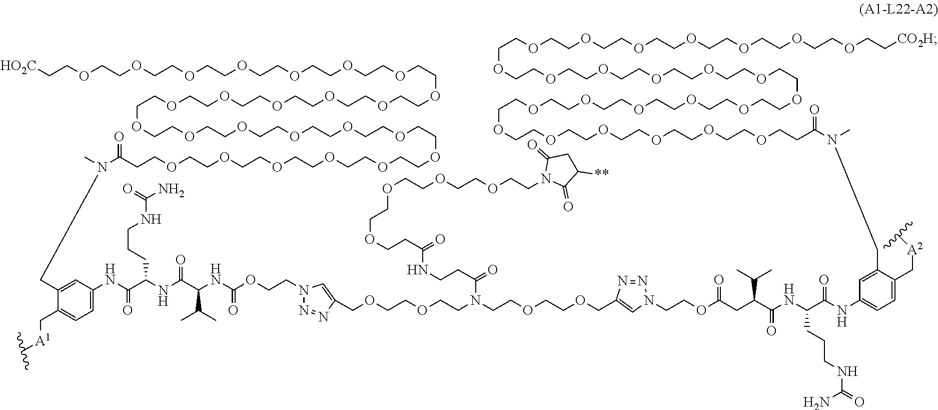

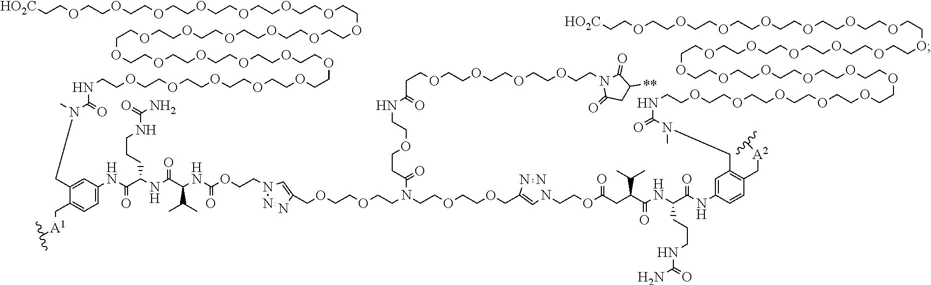

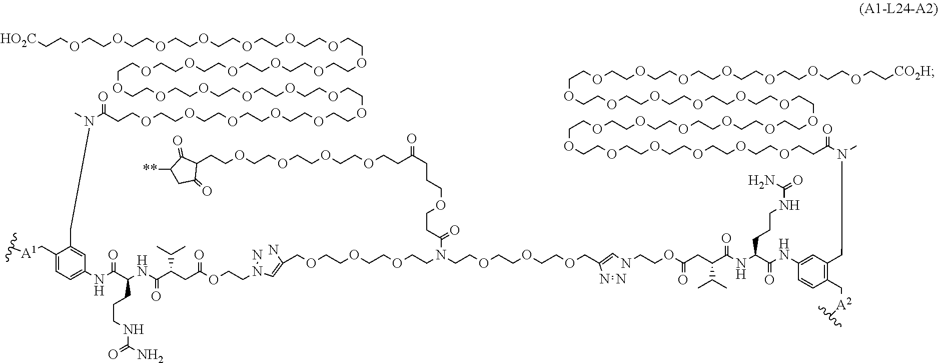

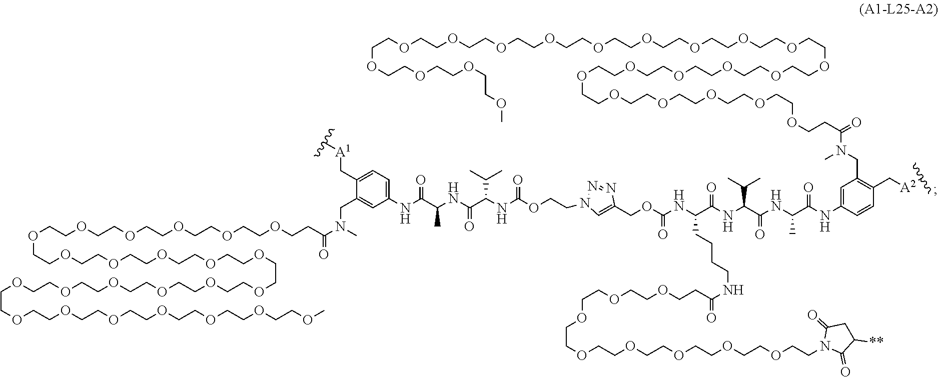

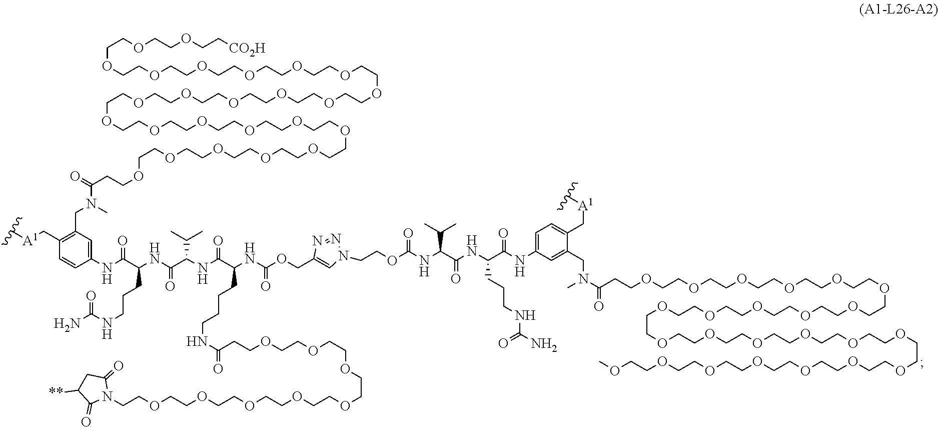

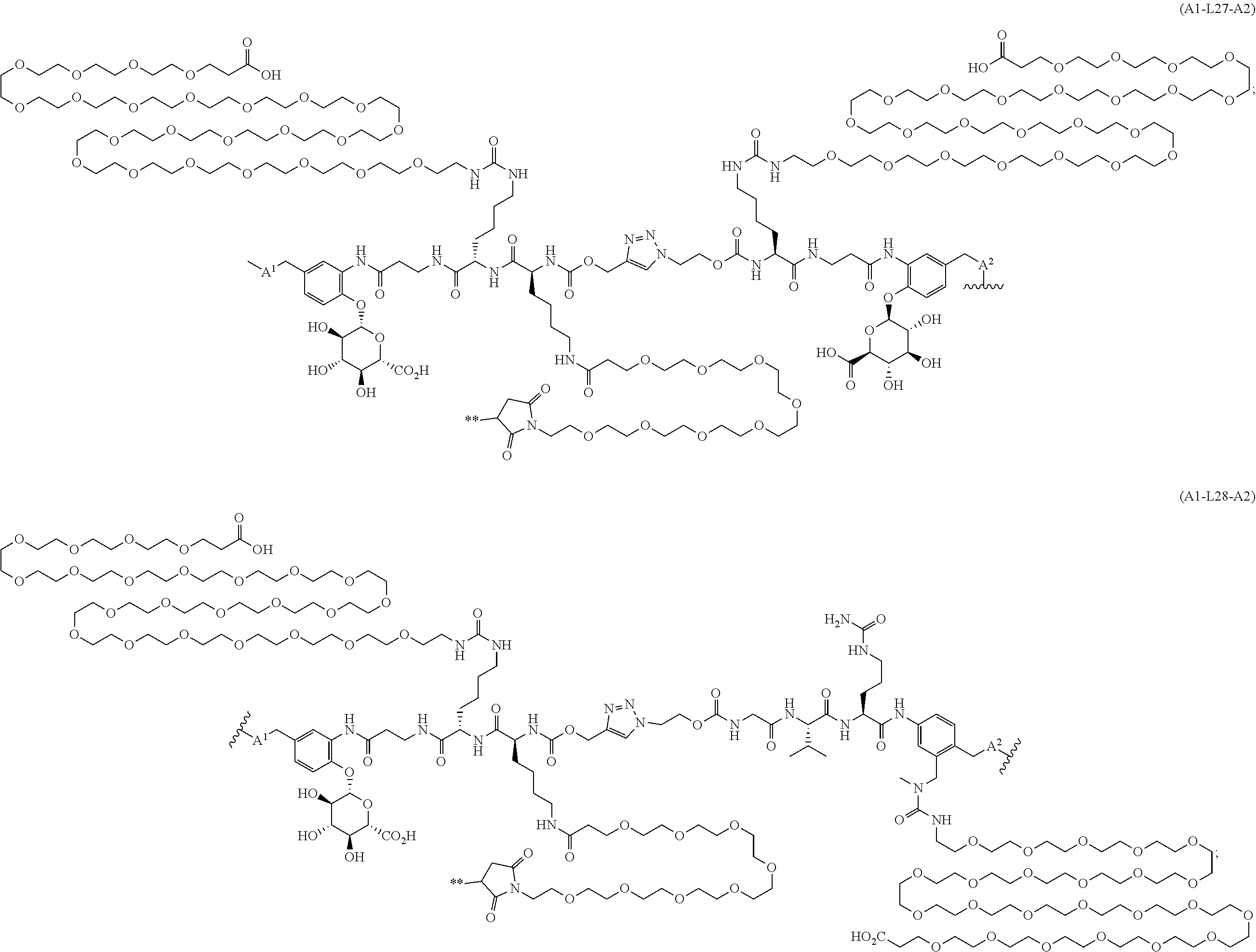

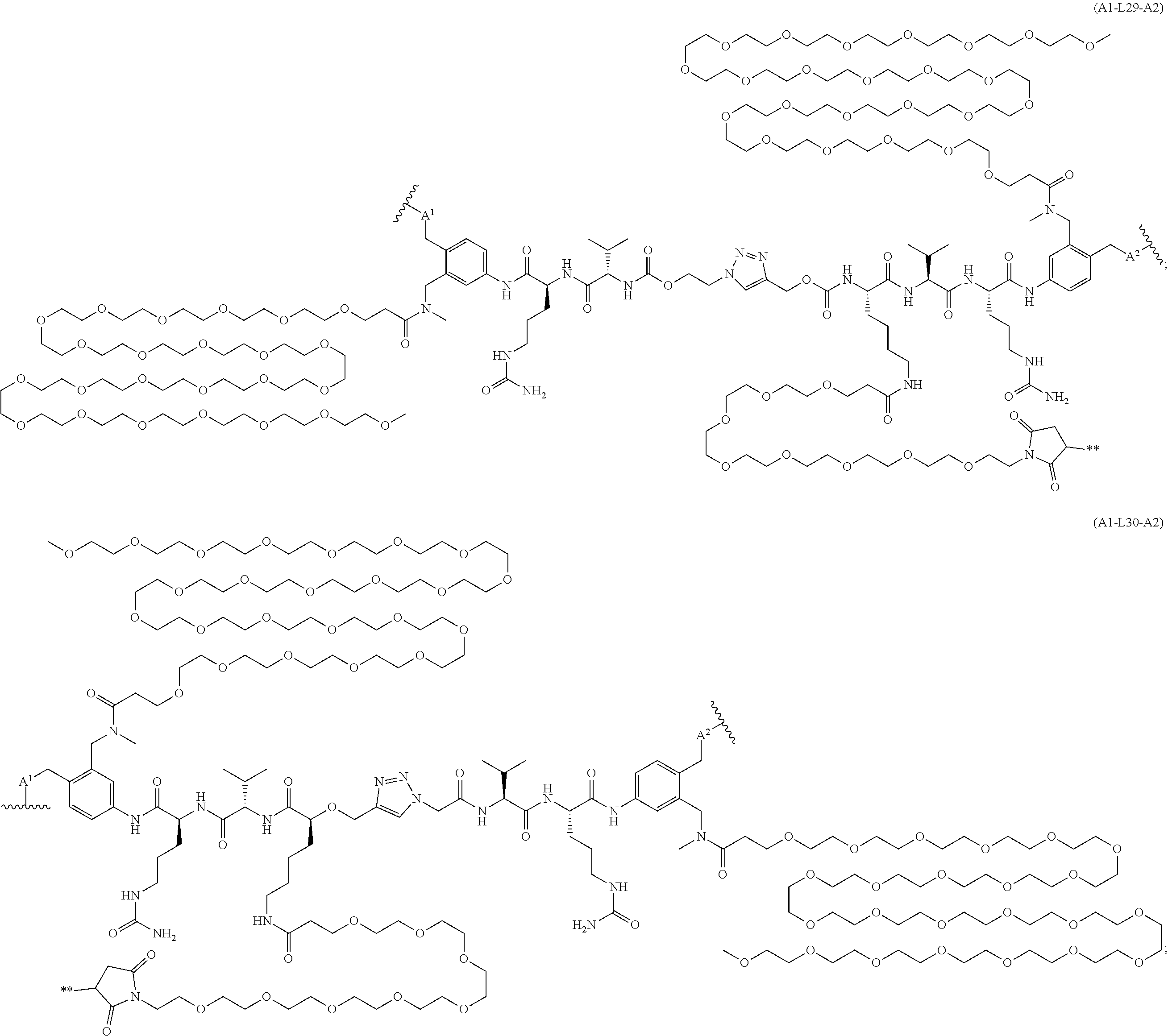

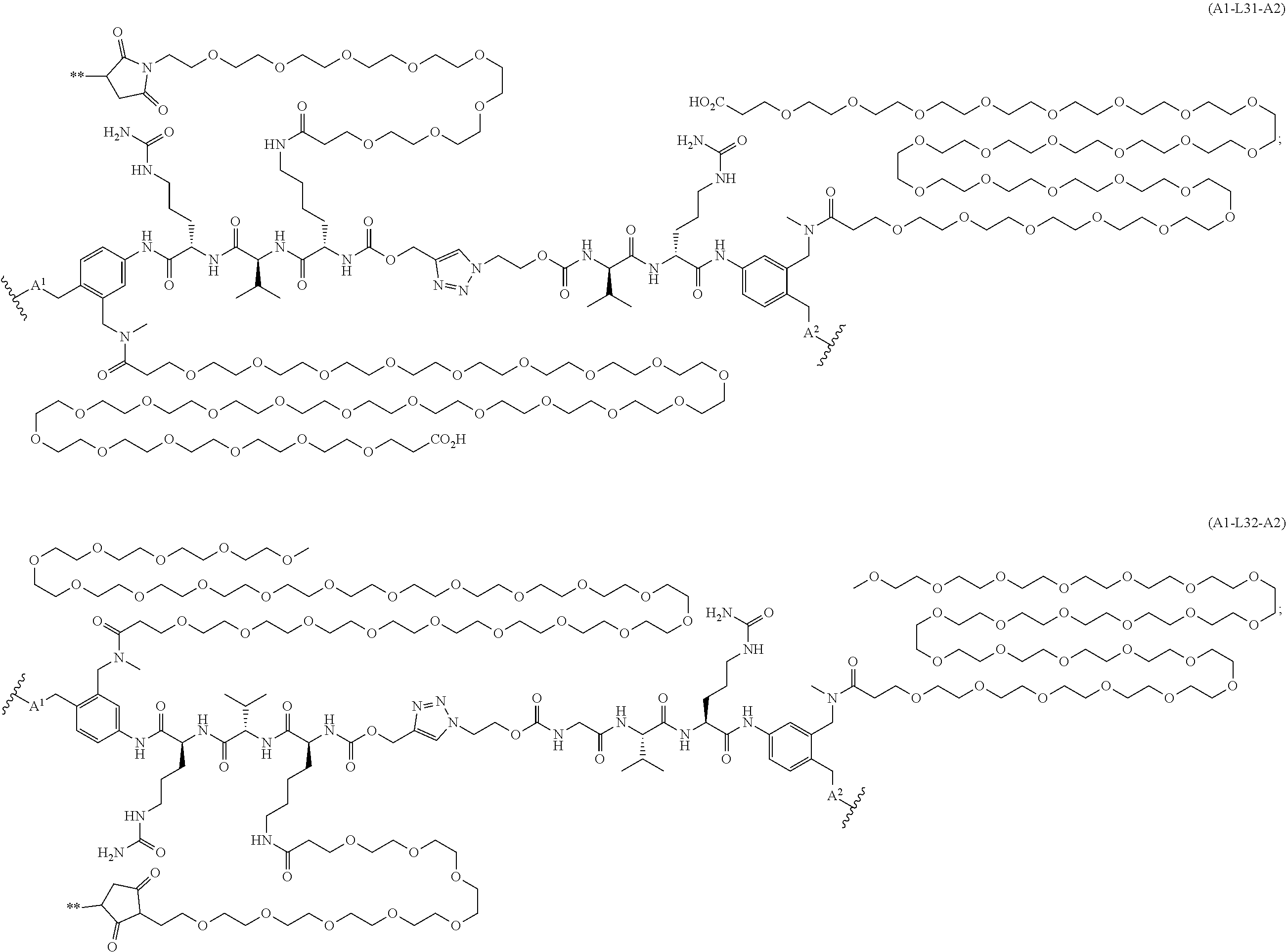

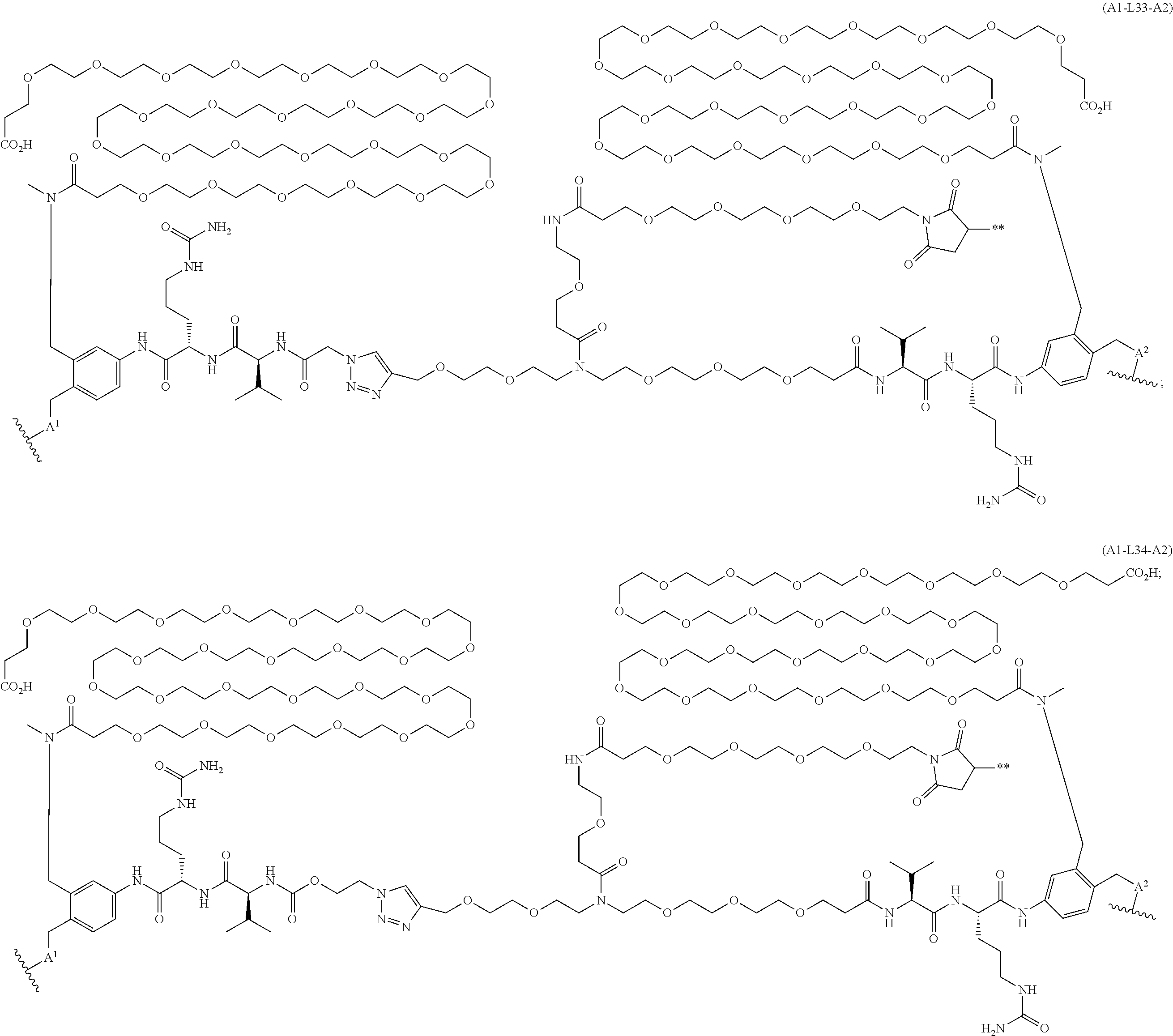

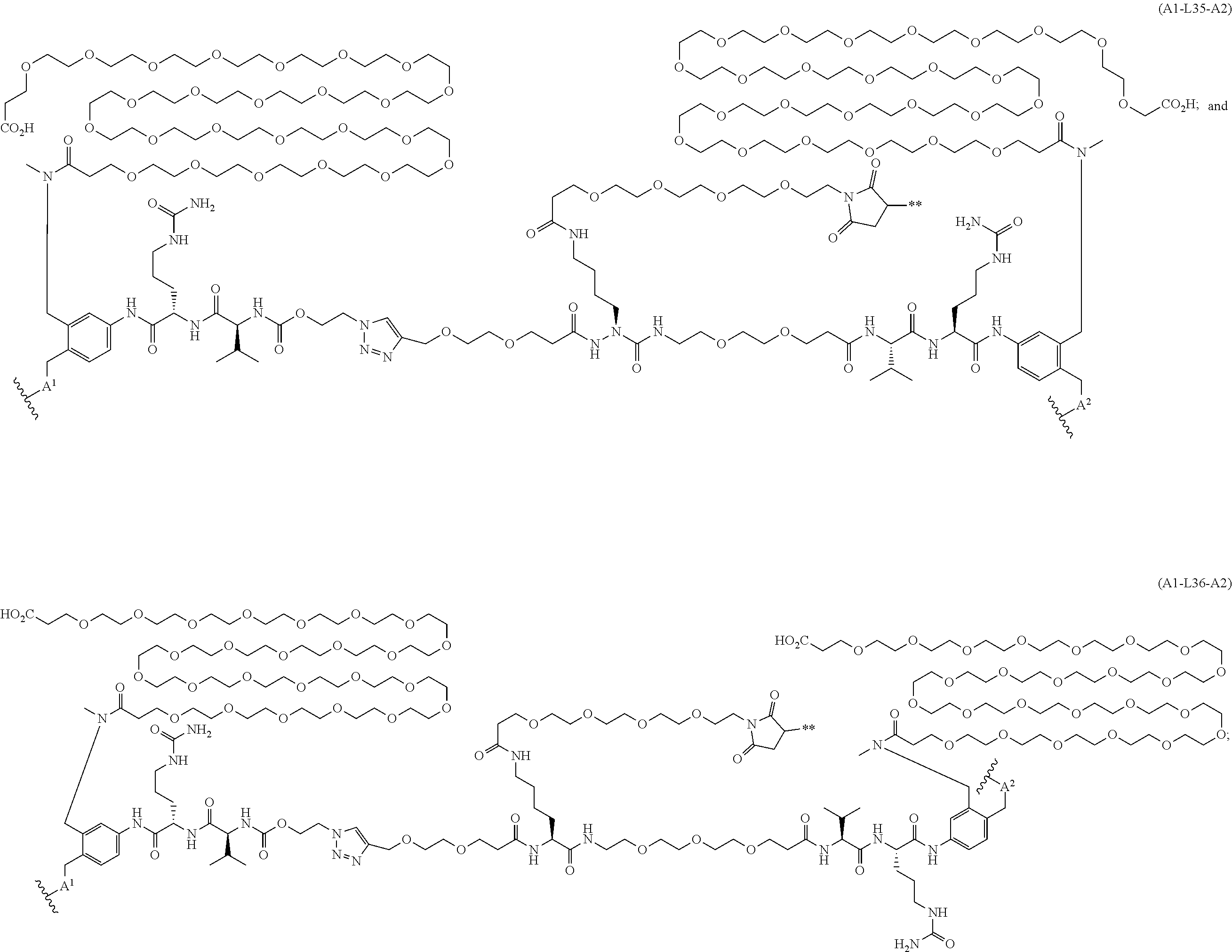

wherein A¹ or A² are each independently selected from a bond,

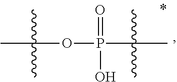 [SMILES: *OP(*)(=O)O]

and —OC(═O)—*, wherein * indicates the point of attachment to D¹ or D²; [symbol] indicates the point of attachment to the Ab; and [symbol] indicates the point of direct attachment to D¹ or D².
Embodiment 116a. The conjugate according to Embodiment 116, wherein A¹ and A² are each independently selected from a bond and —O—C(═O)—*, wherein * in A¹ and A² indicates the point of attachment to D¹ or D².
Embodiment 117. A pharmaceutical composition comprising the conjugate of any one of Embodiments 46 to 110, 113, 115 and 116, and a pharmaceutically acceptable carrier.
Embodiment 118. A method of treating a subject having or suspected of having a cancer, comprising administering to the subject a therapeutically effective amount of the conjugate of any one of Embodiments 46 to 110, 113, 115 and 116, or the pharmaceutical composition of Embodiment 117.
Embodiment 119. The method of Embodiment 118, wherein the cancer expresses a target antigen.
Embodiment 120. The method of Embodiment 118 or Embodiment 119, wherein the cancer is a tumor or a hematological cancer, optionally, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer.
Embodiment 121. A method of reducing or inhibiting the growth of a tumor in a subject, comprising administering to the subject a therapeutically effective amount of the conjugate of any one of Embodiments 46 to 110, 113, 115 and 116, or the pharmaceutical composition of Embodiment 117.
Embodiment 122. The method of Embodiment 121, wherein the tumor expresses a target antigen.
Embodiment 123. The method of Embodiment 121 or Embodiment 122, wherein the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, spleen cancer, pancreatic cancer, stomach cancer, colon cancer, or head and neck cancer.
Embodiment 124. A method of reducing or inhibiting a hematological cancer in a subject, comprising administering to the subject a therapeutically effective amount of the conjugate of any one of Embodiments 46 to 110, 113, 115 and 116, or the pharmaceutical composition of Embodiment 117.
Embodiment 125. The method of Embodiment 124, wherein the hematological cancer expresses a target antigen.
Embodiment 126. The method of Embodiment 124 or Embodiment 125, wherein the hematological cancer is chronic lymphocytic leukemia (CLL), follicular lymphoma, mantle cell lymphoma, diffuse large B-cell lymphoma, acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), Hodgkin's lymphoma, non-Hodgkin's lymphoma or myelodysplasia syndrome (MDS).
Embodiment 127. The method of any one of Embodiments 121 to 126, wherein administration of the conjugate or pharmaceutical composition reduces or inhibits the growth of the tumor or hematological cancer by at least about 10%, at least about 20%, at least about 30%, at least about 40%, at least about 50%, at least about 60%, at least about 70%, at least about 80%, at least about 90%, at least about 95%, or at least about 99%.
Embodiment 128. A method of reducing or slowing the expansion of a cancer cell population in a subject, comprising administering to the subject a therapeutically effective amount of the conjugate of any one of Embodiments 46 to 110, 113, 115 and 116, or the pharmaceutical composition of Embodiment 117.
Embodiment 129. The method of Embodiment 128, wherein the cancer cell population expresses a target antigen.
Embodiment 130. The method of Embodiment 128 or Embodiment 129, wherein the cancer cell population is from a tumor or a hematological cancer, optionally wherein the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer.
Embodiment 131. The method of any one of Embodiments 128 to 130, wherein administration of the conjugate or pharmaceutical composition reduces the cancer cell population or slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%.
Embodiment 132. The method of any one of Embodiments 118 to 131, wherein the conjugate is administered as monotherapy.
Embodiment 133. The method of any one of Embodiments 118 to 131, wherein the conjugate is administered adjunctive to another therapeutic agent or radiation therapy.
Embodiment 134. The method of Embodiment 133, wherein the conjugate is administered in an amount effective to sensitize the tumor cells to one or more additional therapeutic agents and/or radiation therapy.
Embodiment 135. The method of any one of Embodiments 118 to 131, further comprising administering to the subject in need thereof at least one additional therapeutic agent.
Embodiment 136. The method of Embodiment 135, wherein the one additional therapeutic agent is a taxane, a vinca alkaloid, a MEK inhibitor, an ERK inhibitor, topoisomerase inhibitor, or a RAF inhibitor.
Embodiment 137. Use of a conjugate of any one of Embodiments 46 to 110, 113, 115 and 116, or the pharmaceutical composition of Embodiment 117, for the manufacture of a medicament for (i) treating a subject having or suspected of having a cancer, (ii) reducing or inhibiting the growth of a tumor in a subject, (iii) reducing or inhibiting a hematological cancer in a subject, or (iv) reducing or slowing the expansion of a cancer cell population in a subject.
Embodiment 138. A conjugate of any one of Embodiments 46 to 113, 110, 115 and 116, or the pharmaceutical composition of Embodiment 117, for use in (i) treating a subject having or suspected of having a cancer, (ii) reducing or inhibiting the growth of a tumor in a subject, (iii) reducing or inhibiting a hematological cancer in a subject, or (iv) reducing or slowing the expansion of a cancer cell population in a subject.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise. The terms “comprising”, “having”, “being of” as in “being of a chemical formula”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 20%, 15%, 10%, 5%, 1%, 0.5%, or 0.1% of a numerical amount. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

The terms “antibody-drug conjugate,” “antibody conjugate,” “conjugate,” “immunoconjugate,” and “ADC” are used interchangeably, and refer to one or more therapeutic compounds (that is not a BH3-mimetic) that is linked to one or more antibodies or antigen-binding fragments. In some embodiments, the ADC is defined by the generic formula:

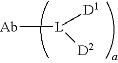

(Formula 1), wherein Ab=an antibody or antigen-binding fragment, L=a dual linker moiety, D¹ and D²=a drug moiety that is not a BH3-mimetic, and a=the number of dual linker moieties with attached D¹ and D² per antibody or antigen-binding fragment.

The term “antibody” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. An antibody can be polyclonal or monoclonal, multiple or single chain, or an intact immunoglobulin, and may be derived from natural sources or from recombinant sources. An “intact” antibody is a glycoprotein that typically comprises at least two heavy (H) chains and two light (L) chains inter-connected by disulfide bonds. Each heavy chain is comprised of a heavy chain variable region (abbreviated herein as VH) and a heavy chain constant region. The heavy chain constant region comprises three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2), or subclass. An antibody can be an intact antibody or an antigen-binding fragment thereof.

In some embodiments, the antibody or antibody fragment disclosed herein include modified or engineered amino acid residues, e.g., one or more cysteine residues, as sites for conjugation to a drug moiety (Junutula J R, et al., Nat Biotechnol 2008, 26:925-932). In one embodiment, the disclosure provides a modified antibody or antibody fragment comprising a substitution of one or more amino acids with cysteine at the positions described herein. Sites for cysteine substitution are in the constant regions of the antibody or antibody fragment and are thus applicable to a variety of antibody or antibody fragment, and the sites are selected to provide stable and homogeneous conjugates. A modified antibody or fragment can have one, two or more cysteine substitutions, and these substitutions can be used in combination with other modification and conjugation methods as described herein. Methods for inserting cysteine at specific locations of an antibody are known in the art, see, e.g., Lyons et al., (1990) Protein Eng., 3:703-708, WO 2011/005481, WO2014/124316, WO 2015/138615. In certain embodiments, a modified antibody comprises a substitution of one or more amino acids with cysteine on its constant region selected from positions 117, 119, 121, 124, 139, 152, 153, 155, 157, 164, 169, 171, 174, 189, 191, 195, 197, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain. In particular embodiments a modified antibody or antibody fragment thereof comprises a combination of substitution of two amino acids with cysteine on its constant regions wherein the combinations comprise substitutions at positions 375 of an antibody heavy chain and position 152 of an antibody heavy chain, wherein the positions are numbered according to the EU system. In particular embodiments a modified antibody or antibody fragment thereof comprises a substitution of one amino acid with cysteine at position 360 of an antibody heavy chain, wherein the positions are numbered according to the EU system. In other particular embodiments a modified antibody or antibody fragment thereof comprises a substitution of one amino acid with cysteine at position 107 of an antibody light chain and wherein the positions are numbered according to the EU system, and wherein the light chain is a kappa chain.

The term “antibody fragment” or “antigen-binding fragment” or “functional antibody fragment,” as used herein, refers to at least one portion of an antibody that retains the ability to specifically interact with (e.g., by binding, steric hinderance, stabilizing/destabilizing, spatial distribution) an epitope of an antigen (e.g., PCAD, HER2, CD48, CD74, EphA2, TROP2, B7-H3 or 5T4). Antigen-binding fragments may also retain the ability to internalize into an antigen-expressing cell. In some embodiments, antigen-binding fragments also retain immune effector activity. The terms antibody, antibody fragment, antigen-binding fragment, and the like, are intended to embrace the use of binding domains from antibodies in the context of larger macromolecules such as ADCs. It has been shown that fragments of a full-length antibody can perform the antigen binding function of a full-length antibody. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, bispecific or multi-specific antibody constructs, ADCs, v-NAR and bis-scFv (see, e.g., Holliger and Hudson (2005) Nat Biotechnol. 23(9):1126-36). Antigen-binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antigen-binding fragment comprising a variable region of a light chain and at least one antigen-binding fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, a scFv may have the VL and VH variable regions in either order, e.g., with respect to the N-terminal and C-terminal ends of the polypeptide, the scFv may comprise VL-linker-VH or may comprise VH-linker-VL. Antigen-binding fragments are obtained using conventional techniques known to those of skill in the art, and the binding fragments are screened for utility (e.g., binding affinity, internalization) in the same manner as are intact antibodies. Antigen-binding fragments, for example, may be prepared by cleavage of the intact protein, e.g., by protease or chemical cleavage.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991) “Sequences of Proteins of Immunological Interest,” 5th Ed. Public Health Service, National Institutes of Health, Bethesda, MD (“Kabat” numbering scheme); AI-Lazikani et al. (1997) J Mol Biol. 273(4):927-48 (“Chothia” numbering scheme); ImMunoGenTics (IMGT) numbering (Lefranc (2001) Nucleic Acids Res. 29(1):207-9; Lefranc et al. (2003) Dev Comp Immunol. 27(1):55-77) (“IMGT” numbering scheme); or a combination thereof. In a combined Kabat and Chothia numbering scheme for a given CDR region (for example, HC CDR1, HC CDR2, HC CDR3, LC CDR1, LC CDR2, or LC CDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.”

In some embodiments, under Kabat, the CDR amino acid residues in the heavy chain variable domain (VH) are numbered 31-35 (HCDR1) (e.g., insertion(s) after position 35), 50-65 (HCDR2), and 95-102 (HCDR3); and the CDR amino acid residues in the light chain variable domain (VL) are numbered 24-34 (LCDR1) (e.g., insertion(s) after position 27), 50-56 (LCDR2), and 89-97 (LCDR3). In some embodiments, under Chothia, the CDR amino acids in the VH are numbered 26-32 (HCDR1) (e.g., insertion(s) after position 31), 52-56 (HCDR2), and 95-102 (HCDR3); and the amino acid residues in VL are numbered 26-32 (LCDR1) (e.g., insertion(s) after position 30), 50-52 (LCDR2), and 91-96 (LCDR3). By combining the CDR definitions of both Kabat and Chothia, in some embodiments, the CDRs comprise or consist of, e.g., amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. In some embodiments, under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3). In some embodiments, under IMGT, the CDR regions of an antibody may be determined using the program IMGT/DomainGap Align.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). Monoclonal antibodies may also be isolated from phage antibody libraries using the techniques described in Clackson et al. (1991) Nature 352:624-8, and Marks et al. (1991) J Mol Biol. 222:581-97, for example. The term also includes preparations of antibody molecules of single molecular composition. A monoclonal antibody composition displays a single binding specificity and affinity for a particular epitope.

The monoclonal antibodies described herein can be non-human, human, or humanized. The term specifically includes “chimeric” antibodies, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity.

The term “human antibody,” as used herein, refers an antibody produced by a human or an antibody having an amino acid sequence of an antibody produced by a human. The term includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik et al. ((2000) J Mol Biol. 296(1):57-86). The structures and locations of immunoglobulin variable domains, e.g., CDRs, may be defined using well known numbering schemes, e.g., the Kabat numbering scheme, the Chothia numbering scheme, or a combination of Kabat and Chothia, and/or ImMunoGenTics (IMGT) numbering. The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody,” as used herein, is not intended to include antibodies in which CDR sequences derived from the germline of another mammalian species, such as a mouse, have been grafted onto human framework sequences.

The term “recombinant human antibody,” as used herein, refers to a human antibody that is prepared, expressed, created, or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody,” as used herein, refers to antibodies wherein the amino acid sequence of the immunoglobulin molecule is derived from two or more species. In some instances, the variable regions of both heavy and light chains correspond to the variable regions of antibodies derived from one species with the desired specificity, affinity, and activity while the constant regions are homologous to antibodies derived from another species (e.g., human) to minimize an immune response in the latter species.

As used herein, the term “humanized antibody” refers to forms of antibodies that contain sequences from non-human (e.g., murine) antibodies as well as human antibodies. Such antibodies are a type of chimeric antibody which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity.

The term “Fc region,” as used herein, refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. In some embodiments, an antibody or antigen-binding fragment comprises an Fc region and a CH1 region of an antibody. In some embodiments, an antibody or antigen-binding fragment comprises an Fc region CH3 region of an antibody. In some embodiments, an antibody or antigen-binding fragment comprises an Fc region, a CH1 region, and a kappa/lambda region from the constant domain of an antibody. In some embodiments, an antibody or antigen-binding fragment comprises a constant region, e.g., a heavy chain constant region and/or a light chain constant region. In some embodiments, such a constant region is modified compared to a wild-type constant region. That is, the polypeptide may comprise alterations or modifications to one or more of the three heavy chain constant domains (CH1, CH2, or CH3) and/or to the light chain constant region domain (CL). Example modifications include additions, deletions, or substitutions of one or more amino acids in one or more domains. Such changes may be included to optimize effector function, half-life, etc. “Internalizing” as used herein in reference to an antibody or antigen-binding fragment refers to an antibody or antigen-binding fragment that is capable of being taken through the cell's lipid bilayer membrane to an internal compartment (i.e., “internalized”) upon binding to the cell, preferably into a degradative compartment in the cell. For example, an internalizing anti-HER2 antibody is one that is capable of being taken into the cell after binding to HER2 on the cell membrane. In some embodiments, the antibody or antigen-binding fragment used in the ADCs disclosed herein targets a cell surface antigen (e.g., PCAD, HER2, CD48, CD74, EphA2, TROP2, B7-H3 or 5T4) and is an internalizing antibody or internalizing antigen-binding fragment (i.e., the ADC transfers through the cellular membrane after antigen binding). In some embodiments, the internalizing antibody or antigen-binding fragment binds a receptor on the cell surface. An internalizing antibody or internalizing antigen-binding fragment that targets a receptor on the cell membrane may induce receptor —mediated endocytosis. In some embodiments, the internalizing antibody or internalizing antigen-binding fragment is taken into the cell via receptor —mediated endocytosis.

“Non-internalizing” as used herein in reference to an antibody or antigen-binding fragment refers to an antibody or antigen-binding fragment that remains at the cell surface upon binding to the cell. In some embodiments, the antibody or antigen-binding fragment used in the ADCs disclosed herein targets a cell surface antigen and is a non-internalizing antibody or non-internalizing antigen-binding fragment (i.e., the ADC remains at the cell surface and does not transfer through the cellular membrane after antigen binding). In some embodiments, the non-internalizing antibody or antigen-binding fragment binds a non-internalizing receptor or other cell surface antigen. Exemplary non-internalizing cell surface antigens include but are not limited to CA125 and CEA, and antibodies that bind to non-internalizing antigen targets are also known in the art (see, e.g., Bast et al. (1981) J Clin Invest. 68(5):1331-7; Scholler and Urban (2007) Biomark Med. 1(4):513-23; and Boudousq et al. (2013) PLoS One 8(7):e69613).

The term “EPH receptor A2” or “EphA2” as used herein, refers to any native form (also known as ephrin type-A receptor 2) of human EphA2. The term encompasses full-length human EphA2 (e.g., NCBI Reference Sequence: NP_004422.2; SEQ ID NO: 337), as well as any form of human EphA2 that may result from cellular processing. The term also encompasses functional variants or fragments of human EphA2, including but not limited to splice variants, allelic variants, and isoforms that retain one or more biologic functions of human EphA2 (i.e., variants and fragments are encompassed unless the context indicates that the term is used to refer to the wild-type protein only). EphA2 can be isolated from human, or may be produced recombinantly or by synthetic methods.

The term “anti-EphA2 antibody” or “antibody that binds to EphA2,” as used herein, refers to any form of antibody or antigen-binding fragment thereof that binds, e.g., specifically binds, to EphA2. The term encompasses monoclonal antibodies (including full-length monoclonal antibodies), polyclonal antibodies, and biologically functional antigen-binding fragments so long as they bind, e.g., specifically bind, to EphA2. WO 2007/030642 provides and is incorporated herein by reference for exemplary EphA2-binding sequences, including exemplary anti-EphA2 antibody sequences. In some embodiments, the anti-EphA2 antibody used in the ADCs disclosed herein is an internalizing antibody or internalizing antigen-binding fragment. 1C1 (WO 2007/030642) is an exemplary anti-EphA2 antibody.

The term “P-cadherin” or “PCAD,” as used herein, refers to any native form of human PCAD (also known as cadherin 3, type 1 or CDH3). The term encompasses full-length human PCAD (e.g., UniProt Reference Sequence: P22223; SEQ ID NO:74), as well as any form of human PCAD that may result from cellular processing. The term also encompasses functional variants or fragments of human PCAD, including but not limited to splice variants, allelic variants, and isoforms that retain one or more biologic functions of human PCAD (i.e., variants and fragments are encompassed unless the context indicates that the term is used to refer to the wild-type protein only). PCAD can be isolated from human, or may be produced recombinantly or by synthetic methods.

The term “anti-PCAD antibody” or “antibody that binds to PCAD,” as used herein, refers to any form of antibody or antigen-binding fragment thereof that binds, e.g., specifically binds, to PCAD. The term encompasses monoclonal antibodies (including full-length monoclonal antibodies), polyclonal antibodies, and biologically functional antigen-binding fragments so long as they bind, e.g., specifically bind, to PCAD. WO 2016/203432 provides and is incorporated herein by reference for exemplary PCAD-binding sequences, including exemplary anti-PCAD antibody sequences. In some embodiments, the anti-PCAD antibody used in the ADCs disclosed herein is an internalizing antibody or internalizing antigen-binding fragment. NOV169N31Q (WO 2016/203432) is an exemplary anti-PCAD antibody.

The term “human epidermal growth factor receptor 2,” “HER2,” or “HER2/NEU,” as used herein, refers to any native form of human HER2. The term encompasses full-length human HER2 (e.g., UniProt Reference Sequence: P04626; SEQ ID NO:75), as well as any form of human HER2 that may result from cellular processing. The term also encompasses functional variants or fragments of human HER2, including but not limited to splice variants, allelic variants, and isoforms that retain one or more biologic functions of human HER2 (i.e., variants and fragments are encompassed unless the context indicates that the term is used to refer to the wild-type protein only). HER2 can be isolated from human, or may be produced recombinantly or by synthetic methods.

The term “anti-HER2 antibody” or “antibody that binds to HER2,” as used herein, refers to any form of antibody or antigen-binding fragment thereof that binds, e.g., specifically binds, to HER2. The term encompasses monoclonal antibodies (including full-length monoclonal antibodies), polyclonal antibodies, and biologically functional antigen-binding fragments so long as they bind, e.g., specifically bind, to HER2. U.S. Pat. Nos. 5,821,337 and 6,870,034 provide and are incorporated herein by reference for exemplary HER2-binding sequences, including exemplary anti-HER2 antibody sequences. In some embodiments, the anti-HER2 antibody used in the ADCs disclosed herein is an internalizing antibody or internalizing antigen-binding fragment. Trastuzumab (U.S. Pat. Nos. 5,821,337 and 6,870,034; see also Molina et al. (2001) Cancer Res. 61(12):4744-9) is an exemplary anti-HER2 antibody.

The term “cluster of differentiation 48” or “CD48,” as used herein, refers to any native form of human CD48 (also known as B-lymphocyte activation marker (BLAST-1) or signaling lymphocytic activation molecule 2 (SLAMF2)). The term encompasses full-length human CD48 (e.g., UniProt Reference Sequence: P09326; SEQ ID NO:77), as well as any form of human CD48 that may result from cellular processing. The term also encompasses functional variants or fragments of human CD48, including but not limited to splice variants, allelic variants, and isoforms that retain one or more biologic functions of human CD48 (i.e., variants and fragments are encompassed unless the context indicates that the term is used to refer to the wild-type protein only). CD48 can be isolated from human, or may be produced recombinantly or by synthetic methods.

The term “anti-CD48 antibody” or “antibody that binds to CD48,” as used herein, refers to any form of antibody or antigen-binding fragment thereof that binds, e.g., specifically binds, to CD48. The term encompasses monoclonal antibodies (including full-length monoclonal antibodies), polyclonal antibodies, and biologically functional antigen-binding fragments so long as they bind, e.g., specifically bind, to CD48. International Patent App. Nos. PCT/IB2021/060871, PCT/US2021/060560, and PCT/US2021/060620 provide and are incorporated herein by reference for exemplary CD48-binding sequences, including exemplary anti-CD48 antibody sequences. In some embodiments, the anti-CD48 antibody used in the ADCs disclosed herein is an internalizing antibody or internalizing antigen-binding fragment. SGN-CD48A (MEM/MEM102) and NY920 are exemplary anti-CD48 antibodies.

The term “cluster of differentiation 74” or “CD74,” as used herein, refers to any native form of human CD74 (also known as HLA class II histocompatibility antigen gamma chain or HLA-DR antigens-associated invariant chain). The term encompasses full-length human CD74 (e.g., NCBI Reference Sequence: NP_001020330.1; SEQ ID NO:140), as well as any form of human CD74 that may result from cellular processing. The term also encompasses functional variants or fragments of human CD74, including but not limited to splice variants, allelic variants, and isoforms that retain one or more biologic functions of human CD74 (i.e., variants and fragments are encompassed unless the context indicates that the term is used to refer to the wild-type protein only). CD74 can be isolated from human, or may be produced recombinantly or by synthetic methods.

The term “anti-CD74 antibody” or “antibody that binds to CD74,” as used herein, refers to any form of antibody or antigen-binding fragment thereof that binds, e.g., specifically binds, to CD74. The term encompasses monoclonal antibodies (including full-length monoclonal antibodies), polyclonal antibodies, and biologically functional antigen-binding fragments so long as they bind, e.g., specifically bind, to CD74. WO2020/236817 provides and is incorporated herein by reference for exemplary CD74-binding sequences, including exemplary anti-CD74 antibody sequences. In some embodiments, the anti-CD74 antibody used in the ADCs disclosed herein is an internalizing antibody or internalizing antigen-binding fragment. Milatuzumab (WO2003/074567) and VHmil×VK1aNQ (WO2020/236817) are an exemplary anti-CD74 antibodies.

The term “binding specificity,” as used herein, refers to the ability of an individual antibody or antigen binding fragment to preferentially react with one antigenic determinant over a different antigenic determinant. The degree of specificity indicates the extent to which an antibody or fragment preferentially binds to one antigenic determinant over a different antigenic determinant. Also, as used herein, the term “specific,” “specifically binds,” and “binds specifically” refers to a binding reaction between an antibody or antigen-binding fragment (e.g., an anti-HER2 antibody, an anti-CD48 antibody, an anti-CD74 antibody, an anti-EphA2 antibody, an anti-PCAD antibody) and a target antigen (e.g., PCAD, HER2, CD48, CD74, EphA2, TROP2, B7-H3 or 5T4) in a heterogeneous population of proteins and other biologics. Antibodies can be tested for specificity of binding by comparing binding to an appropriate antigen to binding to an irrelevant antigen or antigen mixture under a given set of conditions. If the antibody binds to the appropriate antigen with at least 2, 5, 7, 10 or more times more affinity than to the irrelevant antigen or antigen mixture, then it is considered to be specific. A “specific antibody” or a “target-specific antibody” is one that only binds the target antigen (e.g. PCAD, HER2, CD48, CD74, EphA2, TROP2, B7-H3 or 5T4), but does not bind (or exhibits minimal binding) to other antigens. In some embodiments, an antibody or antigen-binding fragment that specifically binds a target antigen (e.g., PCAD, HER2, CD48, CD74, EphA2, TROP2, B7-H3 or 5T4) has a KD of less than 1×10-6 M, less than 1×10-7 M, less than 1×10-8 M, less than 1×10-9 M, less than 1×10-10 M, less than 1×10-11 M, less than 1×10-12 M, or less than 1×10-13 M. In some embodiments, the KD is 1 μM to 500 μM. In some embodiments, the KD is between 500 μM to 1 μM, 1 μM to 100 nM, or 100 mM to 10 nM.

The term “affinity,” as used herein, refers to the strength of interaction between antibody and antigen at single antigenic sites. Without being bound by theory, within each antigen binding site, the variable region of the antibody “arm” interacts through weak non-covalent forces with the antigen at numerous sites; the more interactions, typically the stronger the affinity. The binding affinity of an antibody is the sum of the attractive and repulsive forces operating between the antigenic determinant and the binding site of the antibody.

The term “kon” or “ka” refers to the on-rate constant for association of an antibody to the antigen to form the antibody/antigen complex. The rate can be determined using standard assays, such as a surface plasmon resonance, biolayer inferometry, or ELISA assay.

The term “koff” or “kd” refers to the off-rate constant for dissociation of an antibody from the antibody/antigen complex. The rate can be determined using standard assays, such as a surface plasmon resonance, biolayer inferometry, or ELISA assay.

The term “KD” refers to the equilibrium dissociation constant of a particular antibody-antigen interaction. KD is calculated by ka/kd. The rate can be determined using standard assays, such as a surface plasmon resonance, biolayer inferometry, or ELISA assay.

The term “epitope” refers to the portion of an antigen capable of being recognized and specifically bound by an antibody (or antigen-binding fragment). Epitope determinants generally consist of chemically active surface groupings of molecules such as amino acids or carbohydrate or sugar side chains and can have specific three-dimensional structural characteristics, as well as specific charge characteristics. When the antigen is a polypeptide, epitopes can be formed from contiguous amino acids or noncontiguous amino acids juxtaposed by tertiary folding of the polypeptide. An epitope may be “linear” or “conformational.” Conformational and linear epitopes are distinguished in that the binding to the former but not the latter is lost in the presence of denaturing solvents. The epitope bound by an antibody (or antigen-binding fragment) may be identified using any epitope mapping technique known in the art, including X-ray crystallography for epitope identification by direct visualization of the antigen-antibody complex, as well as monitoring the binding of the antibody to fragments or mutated variations of the antigen, or monitoring solvent accessibility of different parts of the antibody and the antigen. Exemplary strategies used to map antibody epitopes include, but are not limited to, array-based oligo-peptide scanning, limited proteolysis, site-directed mutagenesis, high-throughput mutagenesis mapping, hydrogen-deuterium exchange, and mass spectrometry (see, e.g., Gershoni et al. (2007) BioDrugs 21:145-56; and Hager-Braun and Tomer (2005) Expert Rev Proteomics 2:745-56).

Competitive binding and epitope binning can also be used to determine antibodies sharing identical or overlapping epitopes. Competitive binding can be evaluated using a cross-blocking assay, such as the assay described in “Antibodies, A Laboratory Manual,” Cold Spring Harbor Laboratory, Harlow and Lane (1st edition 1988, 2nd edition 2014). In some embodiments, competitive binding is identified when a test antibody or binding protein reduces binding of a reference antibody or binding protein to a target antigen such as PCAD, HER2, CD48, CD74 EphA2, TROP2, B7-H3 or 5T4 (e.g., a binding protein comprising CDRs and/or variable domains selected from those identified in Tables 3-5), by at least about 50% in the cross-blocking assay (e.g., 50%, 60%, 70%, 80%, 90%, 95%, 99%, 99.5%, or more, or any percentage in between), and/or vice versa. In some embodiments, competitive binding can be due to shared or similar (e.g., partially overlapping) epitopes, or due to steric hindrance where antibodies or binding proteins bind at nearby epitopes (see, e.g., Tzartos, Methods in Molecular Biology (Morris, ed. (1998) vol. 66, pp. 55-66)). In some embodiments, competitive binding can be used to sort groups of binding proteins that share similar epitopes. For example, binding proteins that compete for binding can be “binned” as a group of binding proteins that have overlapping or nearby epitopes, while those that do not compete are placed in a separate group of binding proteins that do not have overlapping or nearby epitopes.

As used herein, the terms “peptide,” “polypeptide,” and “protein” are used interchangeably to refer to a polymer of amino acid residues. The terms encompass amino acid polymers comprising two or more amino acids joined to each other by peptide bonds, amino acid polymers in which one or more amino acid residues is an artificial chemical mimetic of a corresponding naturally-occurring amino acid, as well as naturally-occurring amino acid polymers and non-naturally-occurring amino acid polymers. The terms include, for example, biologically active fragments, substantially homologous polypeptides, oligopeptides, homodimers, heterodimers, variants of polypeptides, modified polypeptides, derivatives, analogs, fusion proteins, among others. The terms also include natural peptides, recombinant peptides, synthetic peptides, or a combination thereof. Unless otherwise indicated, a particular polypeptide sequence also implicitly encompasses conservatively modified variants thereof.

A “recombinant” protein refers to a protein (e.g., an antibody) made using recombinant techniques, e.g., through the expression of a recombinant nucleic acid.

An “isolated” protein refers to a protein unaccompanied by at least some of the material with which it is normally associated in its natural state. For example, a naturally-occurring polynucleotide or polypeptide present in a living organism is not isolated, but the same polynucleotide or polypeptide separated from some or all of the coexisting materials in the living organism, is isolated. The definition includes the production of an antibody in a wide variety of organisms and/or host cells that are known in the art.

An “isolated antibody,” as used herein, is an antibody that has been identified and separated from one or more (e.g., the majority) of the components (by weight) of its source environment, e.g., from the components of a hybridoma cell culture or a different cell culture that was used for its production. In some embodiments, the separation is performed such that it sufficiently removes components that may otherwise interfere with the suitability of the antibody for the desired applications (e.g., for therapeutic use). Methods for preparing isolated antibodies are known in the art and include, without limitation, protein A chromatography, anion exchange chromatography, cation exchange chromatography, virus retentive filtration, and ultrafiltration.

As used herein, the term “variant” refers to a nucleic acid sequence or an amino acid sequence that differs from a reference nucleic acid sequence or amino acid sequence respectively, but retains one or more biological properties of the reference sequence. A variant may contain one or more amino acid substitutions, deletions, and/or insertions (or corresponding substitution, deletion, and/or insertion of codons) with respect to a reference sequence. Changes in a nucleic acid variant may not alter the amino acid sequence of a peptide encoded by the reference nucleic acid sequence, or may result in amino acid substitutions, additions, deletions, fusions, and/or truncations. In some embodiments, a nucleic acid variant disclosed herein encodes an identical amino acid sequence to that encoded by the unmodified nucleic acid or encodes a modified amino acid sequence that retains one or more functional properties of the unmodified amino acid sequence. Changes in the sequence of peptide variants are typically limited or conservative, so that the sequences of the unmodified peptide and the variant are closely similar overall and, in many regions, identical. In some embodiments, a peptide variant retains one or more functional properties of the unmodified peptide sequence. A variant and unmodified peptide can differ in amino acid sequence by one or more substitutions, additions, deletions in any combination.

A variant of a nucleic acid or peptide can be a naturally-occurring variant or a variant that is not known to occur naturally. Variants of nucleic acids and peptides may be made by mutagenesis techniques, by direct synthesis, or by other techniques known in the art. A variant does not necessarily require physical manipulation of the reference sequence. As long as a sequence contains a different nucleic acid or amino acid as compared to a reference sequence, it is considered a “variant” regardless of how it was synthesized. In some embodiments, a variant has high sequence identity (i.e., 60% nucleic acid or amino acid sequence identity or higher) as compared to a reference sequence. In some embodiments, a peptide variant encompasses polypeptides having amino acid substitutions, deletions, and/or insertions as long as the polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% amino acid sequence identity with a reference sequence, or with a corresponding segment (e.g., a functional fragment) of a reference sequence, e.g., those variants that also retain one or more functions of the reference sequence. In some embodiments, a nucleic acid variant encompasses polynucleotides having amino acid substitutions, deletions, and/or insertions as long as the polynucleotide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% nucleic acid sequence identity with a reference sequence, or with a corresponding segment (e.g., a functional fragment) of a reference sequence.

The term “conservatively modified variant” applies to both amino acid and nucleic acid sequences. For nucleic acid sequences, conservatively modified variants refer to those nucleic acids which encode identical or essentially identical amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence. For polypeptide sequences, conservatively modified variants include individual substitutions, deletions, or additions to a polypeptide sequence which result in the substitution of an amino acid with a chemically similar amino acid. Conservative substitutions providing functionally similar amino acids are well known in the art.

The term “conservative sequence modifications,” as used herein, refers to amino acid modifications that do not significantly affect or alter the binding characteristics of, e.g., an antibody or antigen-binding fragment containing the amino acid sequence. Such conservative modifications include amino acid substitutions, additions, and deletions. Modifications can be introduced into an antibody or antigen-binding fragment by standard techniques known in the art, such as, e.g., site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, one or more amino acid residues within an antibody can be replaced with other amino acid residues from the same side chain family and the altered antibody can be tested using the functional assays described herein.

The term “homologous” or “identity,” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions. For example, if half (e.g., five positions in a polymer ten subunits in length) of the positions in two sequences are matched or homologous, the two sequences are 50% homologous; if 90% of the positions (e.g., 9 of 10), are matched or homologous, the two sequences are 90% homologous.

Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. The percent identity between the two sequences is a function of the number of identical positions shared by the sequences, taking into account the number of gaps, and the length of each gap, which need to be introduced for optimal alignment of the two sequences. Generally, the amino acid identity or homology between proteins disclosed herein and variants thereof, including variants of target antigens (such as PCAD, HER2, CD48, CD74, EphA2, TROP2, B7-H3 or 5T4) and variants of antibody variable domains (including individual variant CDRs), is at least 80% to the sequences depicted herein, e.g., identities or homologies of at least 85%, 90%, 91%, 92%, 93%, 94%, 95%, 96%, 97%, 98%, 99%, almost 100%, or 100%.

The comparison of sequences and determination of percent identity between two sequences can be accomplished using a mathematical algorithm. In some embodiments, the percent identity between two amino acid sequences is determined using the Needleman and Wunsch ((1970) J Mol Biol. 48:444-53) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. An exemplary set of parameters is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17) which has been incorporated into the ALIGN program (version 2.0), using a PAM120 weight residue table, a gap length penalty of 12 and a gap penalty of 4.

The term “agent” is used herein to refer to a chemical compound, a mixture of chemical compounds, a biological macromolecule, an extract made from biological materials, or a combination of two or more thereof. The term “therapeutic agent” or “drug” or “pharmaceutically active drug” etc. refers to an agent that is capable of modulating a biological process and/or has biological activity. The “therapeutic agent” or “drug” or “pharmaceutically active drug” may be a chemotherapeutic agent or anti-cancer agent.

Provided that the needed reactive functional group is present, the terms “drug moiety”, “D” or “drug” further refer to chemicals recognized as drugs in the official United States Pharmacopeia, official Homeopathic Pharmacopeia of the United States, or official National Formulary, or any supplement thereof. Exemplary drugs are set forth in the Physician's Desk Reference (PDR) and in the Orange Book maintained by the U.S. Food and Drug Administration (FDA).

In some embodiments, the Drug moiety (D) is an auristatin. Exemplified auristatin drugs include, but are not limited to, the following:

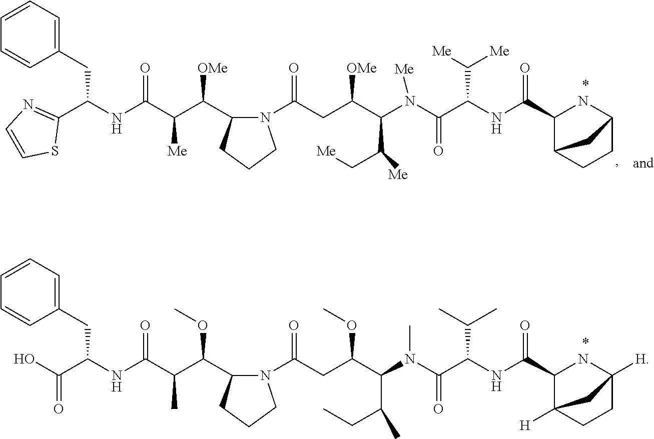

where * indicates the point of attachment to the remainder of the ADC.

The unconjugated forms of these compounds are

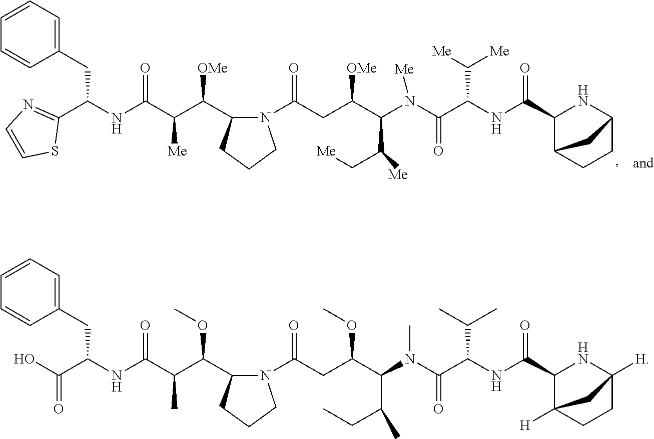

Other exemplary auristatin drugs include, but are not limited to, the following:

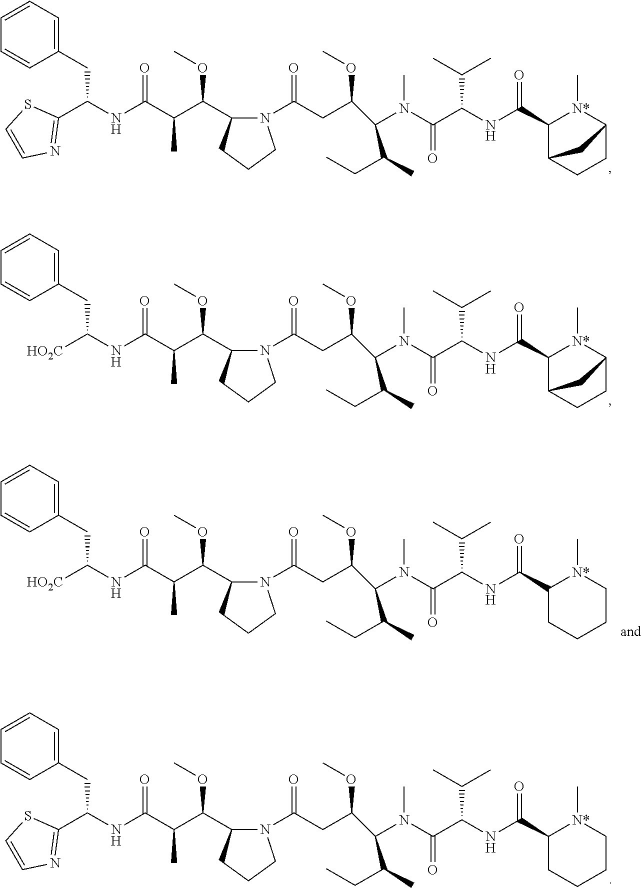

In some embodiments, the Drug moiety (D) is a topoisomerase 1 inhibitor. Exemplified topoisomerase 1 inhibitors include, but are not limited to, the following:

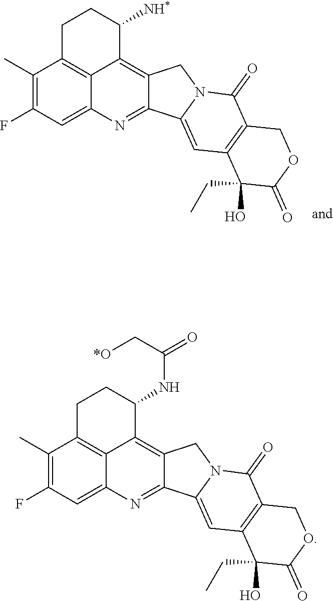

However, the linker technology is broadly applicable to a wide range of drug moieties. In one embodiment, the Drug moiety (D) can be a cytotoxic, cytostatic or immunosuppressive drug. Such cytotoxic or immunosuppressive drugs include, for example, antitubulin agents, tubulin inhibitors, DNA minor groove binders, DNA replication inhibitors, alkylating agents, antibiotics, antifolates, antimetabolites, chemotherapy sensitizers, topoisomerase inhibitors, vinca alkaloids, or the like. Examples of such cytotoxic drugs include, for example, auristatins, camptothecins, duocarmycins, etoposides, maytansines and maytansinoids, taxanes, benzodiazepines or benzodiazepine containing drugs (e.g., pyrrolo[1,4]-benzodiazepines (PBDs), indolinobenzodiazepines, and oxazolidinobenzodiazepines) and vinca alkaloids. In an embodiment, the drug moiety is an targeted anti-cancer compound.

In one embodiment, the drug moiety (D) is an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor, a DHFR inhibitor, or a pro-apoptotic agent, optionally with the proviso that neither D¹ nor D² is a BH3 mimetic.

The effects of the present invention may be more pronounced in embodiments wherein the Drug moiety is hydrophobic. Accordingly, the Drug moiety of the present invention is preferably hydrophobic having a SlogP value of 1.5 or greater, 2.0 or greater, or 2.5 or greater. In some embodiments, drugs to be used in the present invention will have a SlogP value from (a) about 1.5, about 2, or 2.5 to about 7, (b) about 1.5, about 2, or 2.5 to about 6, (c) about 1.5, about 2 or about 2.5 to about 5, (d) about 1.5, about 2, or 2.5 to about 4, or (e) about 1.5, about 2 or about 2.5 to about 3.

Hydrophobicity can be measured using SlogP. SlogP is defined as the log of the octanol/water partition coefficient (including implicit hydrogens) and can be calculated using the program MOE™ from the Chemical Computing group (SlogP values calculated using Wildman, S. A., Crippen, G. M.; Prediction of Physiochemical Parameters by Atomic Contributions; J. Chern. Inf Comput. Sci. 39 No. 5 (1999) 868-873).

The term “chemotherapeutic agent” or “anti-cancer agent” is used herein to refer to all agents that are effective in treating cancer (regardless of mechanism of action). Inhibition of metastasis or angiogenesis is frequently a property of a chemotherapeutic agent. Chemotherapeutic agents include antibodies, biological molecules, and small molecules. A chemotherapeutic agent may be a cytotoxic or cytostatic agent. The term “cytostatic agent” refers to an agent that inhibits or suppresses cell growth and/or multiplication of cells. The term “cytotoxic agent” refers to a substance that causes cell death primarily by interfering with a cell's expression activity and/or functioning.

The term “BH3 mimetics,” as used herein refers to an agent capable of disrupting the interaction between the proapoptotic and antiapoptotic members of the Bcl-2 family and are potent inducers of apoptosis. Exemplary BH3 mimetics includes inhibitors of Bcl-2, Bcl-xL, Bcl-w and Mcl-1.

The term “inhibit” or “inhibition” or “inhibiting,” as used herein, means to reduce a biological activity or process by a measurable amount, and can include but does not require complete prevention or inhibition.

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Often, cancer cells can be in the form of a tumor or mass, but such cells may exist alone within a subject, or may circulate in the blood stream as independent cells, such as leukemic or lymphoma cells. The term “cancer” includes all types of cancers and cancer metastases, including hematological cancers, solid tumors, sarcomas, carcinomas and other solid and non-solid tumor cancers. Hematological cancers may include B-cell malignancies, cancers of the blood (leukemias), cancers of plasma cells (myelomas, e.g., multiple myeloma), or cancers of the lymph nodes (lymphomas). Exemplary B-cell malignancies include chronic lymphocytic leukemia (CLL), follicular lymphoma, mantle cell lymphoma, and diffuse large B-cell lymphoma. Leukemias may include acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), etc. The terms “acute lymphoblastic leukemia” and “acute lymphocytic leukemia” can be used interchangeably to describe ALL. Lymphomas may include Hodgkin's lymphoma, non-Hodgkin's lymphoma, etc. Other hematologic cancers may include myelodysplasia syndrome (MDS). Solid tumors may include carcinomas such as adenocarcinoma, e.g., breast cancer, pancreatic cancer, prostate cancer, colon or colorectal cancer, lung cancer, gastric cancer, cervical cancer, endometrial cancer, ovarian cancer, cholangiocarcinoma, glioma, melanoma, etc. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

As used herein, the term “tumor” refers to any mass of tissue that results from excessive cell growth or proliferation, either benign or malignant, including precancerous lesions. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer, head and neck cancer or spleen cancer. In some embodiments, the tumor is a gastric cancer.

The terms “tumor cell” and “cancer cell” may be used interchangeably herein and refer to individual cells or the total population of cells derived from a tumor or cancer, including both non-tumorigenic cells and cancer stem cells. The terms “tumor cell” and “cancer cell” will be modified by the term “non-tumorigenic” when referring solely to those cells lacking the capacity to renew and differentiate to distinguish those cells from cancer stem cells.

The term “target-negative,” “target antigen-negative,” or “antigen-negative,” as used herein, refers to the absence of target antigen expression by a cell or tissue. The term “target-positive,” “target antigen-positive,” or “antigen-positive” refers to the presence of target antigen expression. For example, a cell or a cell line that does not express a target antigen may be described as target-negative, whereas a cell or cell line that expresses a target antigen may be described as target-positive.

The terms “subject” and “patient” are used interchangeably herein to refer to any human or non-human animal in need of treatment. Non-human animals include all vertebrates (e.g., mammals and non-mammals) such as any mammal. Non-limiting examples of mammals include humans, chimpanzees, apes, monkeys, cattle, horses, sheep, goats, swine, rabbits, dogs, cats, rats, mice, and guinea pigs. Non-limiting examples of non-mammals include birds and fish. In some embodiments, the subject is a human.

The term “a subject in need of treatment,” as used herein, refers to a subject that would benefit biologically, medically, or in quality of life from a treatment (e.g., a treatment with any one or more of the exemplary ADC compounds described herein).

As used herein, the term “treat,” “treating,” or “treatment” refers to any improvement of any consequence of disease, disorder, or condition, such as prolonged survival, less morbidity, and/or a lessening of side effects which result from an alternative therapeutic modality. In some embodiments, treatment comprises delaying or ameliorating a disease, disorder, or condition (i.e., slowing or arresting or reducing the development of a disease or at least one of the clinical symptoms thereof). In some embodiments, treatment comprises delaying, alleviating, or ameliorating at least one physical parameter of a disease, disorder, or condition, including those which may not be discernible by the patient. In some embodiments, treatment comprises modulating a disease, disorder, or condition, either physically (e.g., stabilization of a discernible symptom), physiologically (e.g., stabilization of a physical parameter), or both. In some embodiments, treatment comprises administration of a described ADC compound or composition to a subject, e.g., a patient, to obtain a treatment benefit enumerated herein. The treatment can be to cure, heal, alleviate, delay, prevent, relieve, alter, remedy, ameliorate, palliate, improve, or affect a disease, disorder, or condition (e.g., a cancer), the symptoms of a disease, disorder, or condition (e.g., a cancer), or a predisposition toward a disease, disorder, or condition (e.g., a cancer). In some embodiments, in addition to treating a subject having a disease, disorder, or condition, a composition disclosed herein can also be provided prophylactically to prevent or reduce the likelihood of developing that disease, disorder, or condition.

As used herein, a “pharmaceutical composition” refers to a preparation of a composition, e.g., an ADC compound or composition, in addition to at least one other (and optionally more than one other) component suitable for administration to a subject, such as a pharmaceutically acceptable carrier, stabilizer, diluent, dispersing agent, suspending agent, thickening agent, and/or excipient. The pharmaceutical compositions provided herein are in such form as to permit administration and subsequently provide the intended biological activity of the active ingredient(s) and/or to achieve a therapeutic effect. The pharmaceutical compositions provided herein preferably contain no additional components which are unacceptably toxic to a subject to which the formulation would be administered.

As used herein, the terms “pharmaceutically acceptable carrier” and “physiologically acceptable carrier,” which may be used interchangeably, refer to a carrier or a diluent that does not cause significant irritation to a subject and does not abrogate the biological activity and properties of the administered ADC compound or composition and/or any additional therapeutic agent in the composition. Pharmaceutically acceptable carriers may enhance or stabilize the composition or can be used to facilitate preparation of the composition. Pharmaceutically acceptable carriers can include solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. The carrier may be selected to minimize adverse side effects in the subject, and/or to minimize degradation of the active ingredient(s). An adjuvant may also be included in any of these formulations.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

The term “pharmaceutically acceptable salt,” as used herein, refers to a salt which does not abrogate the biological activity and properties of the compounds of the invention, and does not cause significant irritation to a subject to which it is administered. Examples of such salts include, but are not limited to: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al., “Commentary: Occurrence of Pharmaceutically Acceptable Anions and Cations in the Cambridge Structural Database,” J. Pharmaceutical Sciences, vol. 94, no. 10 (2005), and Berge et al., “Pharmaceutical Salts,” J. Pharmaceutical Sciences, vol. 66, no. 1 (1977), which are incorporated by reference herein.

In some embodiments, depending on their electronic charge, the antibody-drug conjugates (ADCs), linkers, payloads and linker-payloads described herein can contain a monovalent anionic counterion M1−. Any suitable anionic counterion can be used. In certain embodiments, the monovalent anionic counterion is a pharmaceutically acceptable monovalent anionic counterion. In certain embodiments, the monovalent anionic counterion M1− can be selected from bromide, chloride, iodide, acetate, trifluoroacetate, benzoate, mesylate, tosylate, triflate, formate, or the like. In some embodiments, the monovalent anionic counterion M1− is trifluoroacetate or formate.

As used herein, the term “therapeutically effective amount” or “therapeutically effective dose,” refers to an amount of a compound described herein, e.g., an ADC compound or composition described herein, to effect the desired therapeutic result (i.e., reduction or inhibition of an enzyme or a protein activity, amelioration of symptoms, alleviation of symptoms or conditions, delay of disease progression, a reduction in tumor size, inhibition of tumor growth, prevention of metastasis). In some embodiments, a therapeutically effective amount does not induce or cause undesirable side effects. In some embodiments, a therapeutically effective amount induces or causes side effects but only those that are acceptable by a treating clinician in view of a patient's condition. In some embodiments, a therapeutically effective amount is effective for detectable killing, reduction, and/or inhibition of the growth or spread of cancer cells, the size or number of tumors, and/or other measure of the level, stage, progression and/or severity of a cancer. The term also applies to a dose that will induce a particular response in target cells, e.g., a reduction, slowing, or inhibition of cell growth. A therapeutically effective amount can be determined by first administering a low dose, and then incrementally increasing that dose until the desired effect is achieved. A therapeutically effective amount can also vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific amount may vary depending on, for example, the particular pharmaceutical composition, the subject and their age and existing health conditions or risk for health conditions, the dosing regimen to be followed, the severity of the disease, whether it is administered in combination with other agents, timing of administration, the tissue to which it is administered, and the physical delivery system in which it is carried. In the case of cancer, a therapeutically effective amount of an ADC may reduce the number of cancer cells, reduce tumor size, inhibit (e.g., slow or stop) tumor metastasis, inhibit (e.g., slow or stop) tumor growth, and/or relieve one or more symptoms.

As used herein, the term “prophylactically effective amount” or “prophylactically effective dose,” refers to an amount of a compound disclosed herein, e.g., an ADC compound or composition described herein, that is effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In some embodiments, a prophylactically effective amount can prevent the onset of disease symptoms, including symptoms associated with a cancer.

The term “p” or “drug loading” or “drug:antibody ratio” or “drug-to-antibody ratio” or “DAR” refers to the number of drug moieties per antibody or antigen-binding fragment, i.e., drug loading, or the number of drug moieties per antibody or antigen-binding fragment (Ab) in ADCs of Formula (1). In compositions comprising multiple copies of ADCs of Formula (1), “average p” refers to the average number of drug moieties per antibody or antigen-binding fragment, also referred to as “average drug loading.”

The antibody-drug conjugate (ADC) compounds of the present disclosure include those with anti-cancer activity. In particular, the ADC compounds include an antibody or antigen-binding fragment conjugated (i.e., covalently attached by a linker) to a drug moiety, wherein the drug moiety when not conjugated to an antibody or antigen-binding fragment has a cytotoxic or cytostatic effect. Without being bound by theory, by conjugating the drug moiety to an antibody that binds an antigen associated with expression in a tumor cell or cancer, the ADC may provide improved activity, better cytotoxic specificity, and/or reduced off-target killing as compared to the drug moiety when administered alone.

In some embodiments, therefore, the components of the ADC are selected to (i) retain one or more therapeutic properties exhibited by the antibody and drug moieties in isolation, (ii) maintain the specific binding properties of the antibody or antigen-binding fragment; (iii) optimize drug loading and drug-to-antibody ratios; (iv) allow delivery, e.g., intracellular delivery, of the drug moiety via stable attachment to the antibody or antigen-binding fragment; (v) retain ADC stability as an intact conjugate until transport or delivery to a target site; (vi) minimize aggregation of the ADC prior to or after administration; (vii) allow for the therapeutic effect, e.g., cytotoxic effect, of the drug moiety after cleavage or other release mechanism in the cellular environment; (viii) exhibit in vivo anti-cancer treatment efficacy comparable to or superior to that of the antibody and drug moieties in isolation; (ix) minimize off-target killing by the drug moiety; and/or (x) exhibit desirable pharmacokinetic and pharmacodynamics properties, formulatability, and toxicologic/immunologic profiles. Each of these properties may provide for an improved ADC for therapeutic use (Ab et al. (2015) Mol Cancer Ther. 14:1605-13).

The ADC compounds of the present disclosure may selectively deliver an effective dose of a cytotoxic or cytostatic agent to cancer cells or to tumor tissue. In some embodiments, the cytotoxic and/or cytostatic activity of the ADC is dependent on target antigen expression in a cell. In some embodiments, the disclosed ADCs are particularly effective at killing cancer cells expressing a target antigen while minimizing off-target killing. In some embodiments, the disclosed ADCs do not exhibit a cytotoxic and/or cytostatic effect on cancer cells that do not express a target antigen.

Exemplary EphA2-expressing cancers include but are not limited to breast cancer, non-small cell lunch cancer, pancreatic cancer, esophageal cancer, head and neck cancer, stomach cancer, bladder cancer, and colon cancer.

Exemplary PCAD-expressing cancers include but are not limited to breast cancer, gastric cancer, endometrial cancer, ovarian cancer, pancreatic cancer, bladder cancer, prostate cancer, and melanoma (Vieira and Paredes (2015) Mol Cancer 14:178).

Exemplary HER2-expressing cancers include but are not limited to breast cancer, gastric cancer, bladder cancer, urothelial cell carcinoma, esophageal cancer, lung cancer (e.g., lung adenocarcinoma), uterine cancer (e.g., uterine serous endometrial carcinoma), salivary duct carcinoma, cervical cancer, endometrial cancer, and ovarian cancer (English et al. (2013) Mol Diagn Ther. 17:85-99).

Exemplary CD48-expressing cancers include but are not limited to breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, and spleen cancer.

Exemplary CD74-expressing cancers include but are not limited to breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, and spleen cancer.

Provided herein, in certain aspects, are ADC compounds comprising an antibody or antigen-binding fragment thereof (Ab) covalently linked to two pharmaceutically active drugs (D¹ and D²) through a dual linker (L), wherein the dual linker has one attachment point connected to the antibody and two attachment points to the two pharmaceutically active drugs and wherein the two pharmaceutically active drugs can be the same or different. In some embodiments, for the ADC compounds provided herein, the antibody or antigen-binding fragment thereof (Ab) targets a cancer cell. In some embodiments, the antibody or antigen-binding fragment is able to bind to a tumor —associated antigen (e.g., CD74, CD48, EphA2, PCAD, or HER2), e.g., with high specificity and high affinity. In some embodiments, the antibody or antigen-binding fragment is internalized into a target cell upon binding, e.g., into a degradative compartment in the cell. In some embodiments, the ADCs internalize upon binding to a target cell, undergo degradation, and release the pharmaceutically active drugs to kill cancer cells. The pharmaceutically active drugs may be released from the antibody and/or the linker moiety of the ADC by enzymatic action, hydrolysis, oxidation, or any other mechanism.

An exemplary ADC has Formula (1):

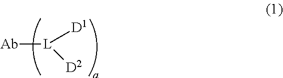

wherein Ab=an antibody or antigen-binding fragment, L=a dual linker moiety, D¹ and D²=a pharmaceutically active drug, and a=the number of pharmaceutically active drugs (D¹ or D²) attached per antibody or antigen-binding fragment.

The antibody or antigen-binding fragment (Ab) of Formula (1) includes within its scope any antibody or antigen-binding fragment that specifically binds to a target antigen on a cell. In some embodiment, the antibody or antigen-binding fragment (Ab) of Formula (1) includes within its scope any antibody or antigen-binding fragment that specifically binds to a target antigen on a cancer cell. The antibody or antigen-binding fragment may bind to a target antigen with a dissociation constant (KD) of ≤1 mM, ≤100 nM or ≤10 nM, or any amount in between, as measured by, e.g., BIAcore® analysis. In some embodiments, the KD is 1 μM to 500 μM. In some embodiments, the KD is between 500 μM to 1 μM, 1 μM to 100 nM, or 100 mM to 10 nM.

In some embodiments, the antibody or antigen-binding fragment is a four-chain antibody (also referred to as an immunoglobulin or a full-length or intact antibody), comprising two heavy chains and two light chains. In some embodiments, the antibody or antigen-binding fragment is an antigen-binding fragment of an immunoglobulin. In some embodiments, the antibody or antigen-binding fragment is an antigen-binding fragment of an immunoglobulin that retains the ability to bind a target cancer antigen and/or provide at least one function of the immunoglobulin.

In some embodiments, the antibody or antigen-binding fragment is an internalizing antibody or internalizing antigen-binding fragment thereof. In some embodiments, the internalizing antibody or internalizing antigen-binding fragment thereof binds to a target cancer antigen expressed on the surface of a cell and enters the cell upon binding. In some embodiments, the drug moiety of the ADC is released from the antibody or antigen-binding fragment of the ADC after the ADC enters and is present in a cell expressing the target cancer antigen (i.e., after the ADC has been internalized), e.g., by cleavage, by degradation of the antibody or antigen-binding fragment, or by any other suitable release mechanism.

In some embodiments, the antibodies comprise mutations that mediate reduced or no antibody-dependent cellular cytotoxicity (ADCC) or complement-dependent cytotoxicity (CDC). In some embodiments, these mutations are known as Fc Silencing, Fc Silent, or Fc Silenced mutations. In some embodiments, amino acid residues L234 and L235 of the IgG1 constant region are substituted to A234 and A235 (also known as “LALA”). In some embodiments, amino acid residue N297 of the IgG1 constant region is substituted to A297 (also known as “N297A”). In some embodiments, amino acid residues D265 and P329 of the IgG1 constant region are substituted to A265 and A329 (also known as “DAPA”). Other antibody Fc silencing mutations may also be used. In some embodiments, the Fc silencing mutations are used in combination, for example D265A, N297A and P329A (also known as “DANAPA”).

As set forth herein, if modifications are made to the antibodies, they are further designated with that modification. For example if select amino acids in the antibody have been changed to cysteines (e.g., E152C, S375C according to EU numbering of the antibody heavy chain to facilitate conjugation to linker-drug moieties) they are designated as “CysMab”; if the antibody has been modified with Fc silencing mutations D265A and P329A of the IgG1 constant region according to EU numbering, “DAPA” is added to the antibody name; or if the antibody has been modified with Fc silencing mutations D265A, N297A and P329A of the IgG1 constant region according to EU numbering, “DANAPA” is added to the antibody name.

Amino acid sequences of exemplary antibodies of the present disclosure, in addition to exemplary antigen targets, are set forth in Tables D1-D8.

TABLE D1
Antibodies Exemplified

| Antibody Target | Antibody Code | mAb Reference |
|---|---|---|
| BCMA | BCMA or Ab B | J6M0 |
| CD33 | CD33ch or Ab G | MuMy9-6ch |
| CD33 | CD33 | gemtuzumab |
| PCAD | PCAD | NOV169N31Q |
| HER2/NEU | HER2 or Ab T | trastuzumab |
| HER2 | HER2 | disitamab |
| CD38 | CD38 | daratumumab |
| CD46 | CD46 | Anti-CD46 |
| CD48 | CD48 | SGN-CD48A |
| CD79b | CD79b | polatuzumab |
| EGFR | Ab C or EGFR1 | cetuximab |
| | CysMab | |
| CD7 | Ab D | Anti-CD7 |
| TFRC | TFRC CysMab | CD71 (CX-2029) |
| EPCAM | EPCAM CysMab | oportuzumab |
| FOLR1 | FOLR1 CysMab | Mirvetuximab |
| ENPP3 | ENPP3 CysMab | ENPP3 (AGS16-7.8) |
| MET | MET CysMab | Telisotuzumab |
| AXL | AXL CysMab | Enapotamab |
| SLC34A2 | SLC34A2 CysMab | Lifastuzumab |
| NECTIN4 | NECTIN4 CysMab | Enfortumab |
| TACSTD2 | TACSTD2 CysMab | Sacituzumab |
| SLC39A6 | SLC39A6 CysMab | Ladiratuzumab |
| GPNMB | GPNMB CysMab | Glembatumumab |
| MSLN | MSLN CysMab | Anetumab |
| CD74 | CD74 CysMab | Milatuzumab |
| CD74 | VHmil × Vk1aNQ | VHmil × VK1aNQ |
| F3/TF | F3 CysMab | Tisotumab |
| MUC16 | MUC16 CysMab | Sofituzumab |
| EGFR | EGFR2 CysMab | Aba |
| CD56 | CD56 CysMab | Lorvotuzumab |
| SEZ6 | SEZ6 CysMab | Anti-SEZ6 (Stemcentrx 17.46) |
| DLL3 | DLL3 CysMab | Rovalpituzumab |
| DLK1 | DLK1 CysMab | Anti-DLK1 (DI-2-14) |
| B7-H3 | B7-H3 CysMab | ABBV-155 |
| B7-H3 | B7-H3 | DS-5573a |
| IgG | IgG | anti-chiLysozyme (3207) |
| PCAD | PCAD CysMab | CQY679 |
| EphA2 | EphA2 CysMab | 1C1 |
| 5T4 | 5T4 Cysmab | |
| Trop2 | Trop2 Cysmab | Datopotamab |

TABLE D2
Amino acid sequences of mAb variable regions

| mAb | IgG chain | SEQ ID NO | Amino acid sequence |
|---|---|---|---|
| J6MO | VH | 1 | QVQLVQSGAEVKKPGSSVKVSCKASGGTFS |
| | | | NYWMHWVRQAPGQGLEWMGATYRGHSDTYY |
| | | | NQKFKGRVTITADKSTSTAYMELSSLRSED |
| | | | TAVYYCARGAIYNGYDVLDNWGQGTLVTVS |
| | | | S |
| J6MO | VL | 2 | DIQMTQSPSSLSASVGDRVTITCSASQDIS |
| | | | NYLNWYQQKPGKAPKLLIYYTSNLHSGVPS |
| | | | RFSGSGSGTDFTLTISSLQPEDFATYYCQQ |
| | | | YRKLPWTFGQGTKLEIK |
| MuMy9-6ch | VH | 3 | QVQLQQPGAEVVKPGASVKMSCKASGYTFT |
| | | | SYYIHWIKQTPGQGLEWVGVIYPGNDDISY |
| | | | NQKFKGKATLTADKSSTTAYMQLSSLTSED |
| | | | SAVYYCAREVRLRYFDVWGAGTTVTVSS |
| MuMy9-6ch | VL | 4 | NIMLTQSPSSLAVSAGEKVTMSCKSSQSVF |
| | | | FSSSQKNYLAWYQQIPGQSPKLLIYWASTR |
| | | | ESGVPDRFTGSGSGTDFTLIISSVQSEDLA |
| | | | IYYCHQYLSSRTFGGGTKLEIK |
| gemtuzumab | VH | 5 | EVQLVQSGAEVKKPGSSVKVSCKASGYTIT |
| | | | DSNIHWVRQAPGQSLEWIGYIYPYNGGTDY |
| | | | NQKFKNRATLTVDNPTNTAYMELSSLRSED |
| | | | TAFYYCVNGNPWLAYWGQGTLVTVSS |
| gemtuzumab | VL | 6 | DIQLTQSPSTLSASVGDRVTITCRASESLD |
| | | | NYGIRFLTWFQQKPGKAPKLLMYAASNQGS |
| | | | GVPSRFSGSGSGTEFTLTISSLQPDDFATY |
| | | | YCQQTKEVPWSFGQGTKVEVK |
| NOV169N31Q | VH | 7 | QVQLQQSGPGLVKPSQTLSLTCAISGDSVS |
| | | | SQSAAWNWIRQSPSRGLEWLGRIYYRSKWY |
| | | | NDYALSVKSRITINPDTSKNQFSLQLNSVT |
| | | | PEDTAVYYCARGEGYGREGFAIWGQGTLVT |
| | | | VSS |
| NOV169N31Q | VL | 8 | DIQMTQSPSSLSASVGDRVTITCRASQTIS |
| | | | NTLAWYQQKPGKAPKLLIYAASNLQSGVPS |
| | | | RFSGSGSGTDFTLTISSLQPEDFATYYCQQ |
| | | | YLSWFTFGQGTKVEIK |
| trastuzumab | VH | 9 | EVQLVESGGGLVQPGGSLRLSCAASGFNIK |
| | | | DTYIHWVRQAPGKGLEWVARIYPTNGYTRY |
| | | | ADSVKGRFTISADTSKNTAYLQMNSLRAED |
| | | | TAVYYCSRWGGDGFYAMDYWGQGTLVTVSS |
| trastuzumab | VL | 10 | DIQMTQSPSSLSASVGDRVTITCRASQDVN |
| | | | TAVAWYQQKPGKAPKLLIYSASFLYSGVPS |
| | | | RFSGSRSGTDFTLTISSLQPEDFATYYCQQ |
| | | | HYTTPPTFGQGTKVEIKRT |
| daratumumab | VH | 11 | EVQLLESGGGLVQPGGSLRLSCAVSGFTFN |
| | | | SFAMSWVRQAPGKGLEWVSAISGSGGGTYY |
| | | | ADSVKGRFTISRDNSKNTLYLQMNSLRAED |
| | | | TAVYFCAKDKILWFGEPVFDYWGQGTLVTV |
| | | | SS |
| daratumumab | VL | 12 | EIVLTQSPATLSLSPGERATLSCRASQSVS |
| | | | SYLAWYQQKPGQAPRLLIYDASNRATGIPA |
| | | | RFSGSGSGTDFTLTISSLEPEDFAVYYCQQ |
| | | | RSNWPPTFGQGTKVEI |
| SGN-48A | VH | 13 | QVQLVQSGSELKKPGASVKVSCKASGYTFT |
| | | | DFGMNWVRQAPGQGLEWMGWINTFTGEPSY |
| | | | GNVFKGRFVFSLDTSVSTAYLQISSLKAED |
| | | | TAVYYCARRHGNGNVFDSWGQGTLVTVSS |
| SGN-48A | VL | 14 | EIVLTQSPDFQSVTPKEKVTITCRASQSIG |
| | | | SNIHWYQQKPDQSPKLLIKYTSESISGVPS |
| | | | RFSGSGSGTDFTLTINSLEAEDAATYYCQQ |
| | | | SNSWPLTFGGGTKVEIK |
| polatuzumab | VH | 80 | EVQLVESGGGLVQPGGSLRLSCAASGYTFS |
| | | | SYWIEWVRQAPGKGLEWIGEILPGGGDTNY |
| | | | NEIFKGRATFSADTSKNTAYLQMNSLRAED |
| | | | TAVYYCTRRVPIRLDYWGQGTLVTVSS |
| polatuzumab | VL | 81 | DIQLTQSPSSLSASVGDRVTITCKASQSVD |
| | | | YEGDSFLNWYQQKPGKAPKLLIYAASNLES |
| | | | GVPSRFSGSGSGTDFTLTISSLQPEDFATY |
| | | | YCQQSNEDPLTFGQGTKVEIK |
| Anti-CD46 | VH | 90 | QVQLVQSGGGVVQPGRSLRLACAASGLTVN |
| | | | NYAMHWVRQAPGKGLEWVAVISYDGNNKYY |
| | | | ADSVKGRFTISRDNSKNTLYLQ |
| | | | MNSLRAEDTAVYYCAKGGGYFDLWGRGTLV |
| | | | TVSS |
| Anti-CD46 | VL | 91 | QSVLTQPPSVSGAPGQRVTISCTGSSSNIG |
| | | | AGYDVHWYQQLPGTAPKLLIYGNNNRP |
| | | | SGVPDRFSGSKSGTSASLAITGLQAEDEA |
| | | | DYYCSSYTSGTWLFGGGTKLTVL |
| Cetuximab | VH | 151 | EVQLQESGPGLVKPSQTLSLTCTVSGYSIS |
| | | | RDFAWNWIRQPPGKGLEWMGYISYNGNTRY |
| | | | QPSLKSRITISRDTSKNQFFLKLNSVTAAD |
| | | | TATYYCVTASRGFPYWGQGTLVTVSS |
| Cetuximab | VL | 152 | DIQMTQSPSSMSVSVGDRVTITCHSSQDIN |
| | | | SNIGWLQQKPGKSFKGLIYHGTNLDDGVPS |
| | | | RFSGSGSGTDYTLTISSLQPEDFATYYCVQ |
| | | | YAQFPWTFGGGTKIEIK |
| VHmil x | VH | 153 | QVQLQQSGSELKKPGASVKVSCKASGYTFT |
| VK1aNQ | | | NYGVNWIKQAPGQGLQWMGWINPNTGEPTF |
| | | | DDDFKGRFAFSLDTSVSTAYLQISSLKADD |
| | | | TAVYFCSRSRGKNEAWFAYWGQGTLVtVSS |
| VHmil x | VL | 154 | DIVMTQTPLSLPVTPGEPASISCRSSQSLV |
| VK1aNQ | | | HRNQNTYLHWYLQKPGQSPQLLIYTVSNRF |
| | | | SGVPDRFSGSGSGTDFTLKISRVEAEDVGV |
| | | | YFCSQSSHVPPTFGQGTKLEIK |
| SEZ6 | VH | 155 | QVQLVQSGAEVKKPGASVKVSCKASGYTFT |
| | | | SYWINWVRQAPGQGLEWIGNIFPDTTTTNY |
| | | | NEKFKGRVTLTRDTSISTAYMELSRLRSDD |
| | | | TAVYYCAREYYDGTYDAMDYWGQGTLVTVS |
| | | | S |
| SEZ6 | VL | 156 | AIQMTQSPSSLSASVGDRVTITCKASQSVN |
| | | | NDVAWYQQKPGKAPKLLIYYASNRYTGVPS |
| | | | RFSGSGSGTDFTLTISSLQPEDFATYFCQQ |
| | | | DYSSPRTFGQGTKLEIK |
| CD56 | VH | 157 | QVQLVESGGGVVQPGRSLRLSCAASGFTFS |
| | | | SFGMHWVRQAPGKGLEWVAYISSGSFTIYY |
| | | | ADSVKGRFTISRDNSKNTLYLQMNSLRAED |
| | | | TAVYYCARMRKGYAMDYWGQGTLVTVSS |
| CD56 | VL | 158 | DVVMTQSPLSLPVTLGQPASISCRSSQIII |
| | | | HSDGNTYLEWFQQRPGQSPRRLIYKVSNRF |
| | | | SGVPDRFSGSGSGTDFTLKISRVEAEDVGV |
| | | | YYCFQGSHVPHTFGQGTKVEIK |
| DLL3 | VH | 159 | QVQLVQSGAEVKKPGASVKVSCKASGYTFT |
| | | | NYGMNWVRQAPGQGLEWMGWINTYTGEPTY |
| | | | ADDFKGRVTMTTDTSTSTAYMELRSLRSDD |
| | | | TAVYYCARIGDSSPSDYWGQGTLVTVSS |
| DLL3 | VL | 160 | EIVMTQSPATLSVSPGERATLSCKASQSVS |
| | | | NDVVWYQQKPGQAPRLLIYYASNRYTGIPA |
| | | | RFSGSGSGTEFTLTISSLQSEDFAVYYCQQ |
| | | | DYTSPWTFGQGTKLEIK |
| DLK1 | VH | 161 | EVQLQQSGAELVKPGASVKLSCTASGFNIR |
| | | | DTYIHWVKQRPEQGLEWIGRIDPPNGNLKY |
| | | | DPKFQGKATITADTSSNTAYLQFSSLTSED |
| | | | TAVYYCARSDGYSFAYWGQGTLVTVSS |
| DLK1 | VL | 162 | DIVMTQAAPSVPVTPGESVSISCRSSKSLL |
| | | | HSNGNTYLYWFLQRPGQSPQLLIYRMSNLA |
| | | | SGVPDRFSGSGSGTAFTLRISRVEAEDVGV |
| | | | YYCMQHVEYPFTFGSGTKLEIK |
| B7-H3 | VH | 163 | EVQLVQSGAEVKKPGSSVKVSCKASGYTFS |
| ABBV-155 | | | SYWMHWVRQAPGQGLEWIGLIHPESGSTNY |
| | | | NEMFKNRATLTVDRSTSTAYMELSSLRSED |
| | | | TAVYYCAGGGRLYFDYWGQGTTVTVSS |
| B7-H3 | VL | 164 | DIVMTQSPLSLPVTPGEPASISCRSSQSLV |
| ABBV-155 | | | HSNRDTYLRWYLQKPGQSPQLLIYKVSNRF |
| | | | SGVPDRFSGSGSGTDFTLKISRVEAEDVGV |
| | | | YYCSQSTHVPYTFGGGTKVEIK |
| B7-H3 | VH | 165 | QVQLVQSGAEVKKPGSSVKVSCKASGYTFT |
| DS-5573a | | | NYVMHWVRQAPGQGLEWMGYINPYNDDVKY |
| | | | NEKFKGRVTITADESTSTAYMELSSLRSED |
| | | | TAVYYCARWGYYGSPLYYFDYWGQGTLVTV |
| | | | SS |
| B7-H3 | VL | 166 | EIVLTQSPATLSLSPGERATLSCRASSRLI |
| DS-5573a | | | YMHWYQQKPGQAPRPLIYATSNLASGIPAR |
| | | | FSGSGSGTDFTLTISSLEPEDFAVYYCQQW |
| | | | NSNPPTFGQGTKVEIK |
| IgG | VH | 167 | QVQLQQSGPGLVKPSQTLSLTCAISGDSVS |
| | | | SNSAAWSWIRQSPGRGLEWLGRIYYRSKWY |
| | | | NDYAVSVKSRITINPDTSKNQFSLQLNSVT |
| | | | PEDTAVYYCARLDHRYHEDTVYPGMDVWGQ |
| | | | GTLVTVSS |
| IgG | VL | 168 | DIELTQPPSVSVAPGQTARISCSGDNLPAY |
| | | | TVTWYQQKPGQAPVLVIYDDSDRPSGIPER |
| | | | FSGSNSGNTATLTISGTQAEDEADYYCASW |
| | | | DPSSGVVFGGGTKLTVL |
| HER2 | VH | 338 | EVQLVQSGAEVKKPGATVKISCKVSGY |
| Disitamab | | | TFTDYYIHWVQQAPGKGLEWMGRVNP |
| | | | DHGDSYYNQKFKDKATITADKSTDTAY |
| | | | MELSSLRSEDTAVYFCARNYLFDHWGQ |
| | | | GTLVTVSS |
| HER2 | VL | 339 | DIQMTQSPSSVSASVGDRVTITCKASQ |
| Disitamab | | | DVGTAVAWYQQKPGKAPKLLIYWASIR |
| | | | HTGVPSRFSGSGSGTDFTLTISSLQPE |
| | | | DFATYYCHQFATYTFGGGTKVEIK |

TABLE D2a
Nucleotide sequence corresponding to
the variable domain heavy and light
chain (VH and VL) amino acid sequences
of the HER2 Disitamab antibody

| | | SEQ | |
|---|---|---|---|
| | IgG | ID | |
| mAb | chain | NO | Nucleotide sequence |
| HER2 | VH | 340 | gaagttcagctggttcagtct |
| Disitamab | | | ggcgccgaagtgaagaaacct |
| | | | ggcgccaccgtgaagatcagc |
| | | | tgcaaggtgtccggctacacc |
| | | | ttcaccgactactacatccac |
| | | | tgggtgcagcaggcccctggc |
| | | | aaaggacttgagtggatgggc |
| | | | agagtgaaccccgatcacggc |
| | | | gacagctactacaaccagaag |
| | | | ttcaaggacaaggccaccatc |
| | | | accgccgacaagagcaccgat |
| | | | accgcctacatggaactgagc |
| | | | agcctgagaagcgaggatacc |
| | | | gccgtgtacttctgcgcccgg |
| | | | aactacctgtttgaccactgg |
| | | | ggacagggcaccctggtcaca |
| | | | gttagttct |
| HER2 | VL | 341 | gacattcagatgacacagagc |
| Disitamab | | | cctagcagcgtgtccgcctct |
| | | | gtgggagacagagtgaccatc |
| | | | acatgcaaggccagccaggat |
| | | | gtgggaacagccgtggcttgg |
| | | | tatcagcagaagcctggcaag |
| | | | gcccctaagctgctgatctac |
| | | | tgggccagcatcagacacaca |
| | | | ggcgtgcccagcagattttct |
| | | | ggcagcggctctggcaccgac |
| | | | ttcaccctgaccatatctagc |
| | | | ctgcagccagaggacttcgcc |
| | | | acctactactgccaccagttt |
| | | | gccacctacaccttcggcgga |
| | | | ggcaccaaggtggaaatcaag |

TABLE D3
Amino acid sequences of mAb CDRs (Combined)

| | | SEQ | |
|---|---|---|---|
| | IgG | ID | Amino acid |
| mAb | chain | NO | sequence |
| J6MO | HCDR1 | 15 | GGTFSNYWMH |
| J6MO | HCDR2 | 16 | ATYRGHSDTYYNQKFKG |
| J6MO | HCDR3 | 17 | GAIYNGYDVLDN |
| J6MO | LCDR1 | 18 | SASQDISNYLN |
| J6M0 | LCDR2 | 19 | YTSNLHS |
| J6MO | LCDR3 | 20 | QQYRKLPWT |
| MuMy9-6ch | HCDR1 | 21 | GYTFTSYYIH |
| MuMy9-6ch | HCDR2 | 22 | VIYPGNDDISYNQKFKG |
| MuMy9-6ch | HCDR3 | 23 | EVRLRYFDV |
| MuMy9-6ch | LCDR1 | 24 | KSSQSVFFSSSQKNYLA |
| MuMy9-6ch | LCDR2 | 25 | WASTRES |
| MuMy9-6ch | LCDR3 | 26 | HQYLSSRT |
| gemtuzumab | HCDR1 | 27 | GYTITDSNIH |
| gemtuzumab | HCDR2 | 28 | YIYPYNGGTDYNQKFKN |
| gemtuzumab | HCDR3 | 29 | GNPWLAY |
| gemtuzumab | LCDR1 | 30 | RASESLDNYGIRFLT |
| gemtuzumab | LCDR2 | 31 | AASNQGS |
| gemtuzumab | LCDR3 | 32 | QQTKEVPWS |
| NOV169N31Q | HCDR1 | 33 | TCAISGDSVSSQSAAWN |
| NOV169N31Q | HCDR2 | 34 | RIYYRSKWYNDYALSVKS |
| NOV169N31Q | HCDR3 | 35 | GEGYGREGFAI |
| NOV169N31Q | LCDR1 | 36 | RASQTISNTLA |
| NOV169N31Q | LCDR2 | 37 | AASNLQS |
| NOV169N31Q | LCDR3 | 38 | QQYLSWFT |
| trastuzumab | HCDR1 | 39 | GFNIKDTYIH |
| trastuzumab | HCDR2 | 40 | RIYPTNGYTRYADSVKG |
| trastuzumab | HCDR3 | 41 | WGGDGFYAMDV |
| trastuzumab | LCDR1 | 42 | RASQDVNTAVAW |
| trastuzumab | LCDR2 | 43 | SASFLES |
| trastuzumab | LCDR3 | 44 | QQHYTTPPT |
| daratumumab | HCDR1 | 45 | GFTFNSFAMS |
| daratumumab | HCDR2 | 46 | AISGSGGGTYYADSVKG |
| daratumumab | HCDR3 | 47 | DKILWFGEPVFDY |
| daratumumab | LCDR1 | 48 | RASQSVSSYLA |
| daratumumab | LCDR2 | 49 | DASNRAT |
| daratumumab | LCDR3 | 50 | QQRSNWPPT |
| SGN-48A | HCDR1 | 51 | GYTFTDFGMN |
| SGN-48A | HCDR2 | 52 | WINTFTGEPSYGNVFKG |
| SGN-48A | HCDR3 | 53 | RHGNGNVFDS |
| SGN-48A | LCDR1 | 54 | RASQSIGSNIH |
| SGN-48A | LCDR2 | 55 | YTSESIS |
| SGN-48A | LCDR3 | 56 | QQSNSWPLT |
| polatuzumab | HCDR1 | 82 | GYTFSSYWIE |
| polatuzumab | HCDR2 | 83 | EILPGGGDTNYNEIFKG |
| polatuzumab | HCDR3 | 84 | RVPIRLDY |
| polatuzumab | LCDR1 | 85 | ITCKASQSVDYEGDSFLN |
| polatuzumab | LCDR2 | 86 | AASNLES |
| polatuzumab | LCDR3 | 87 | QQSNEdPLT |
| VHmil x VK1aNQ | HCDR1 | 169 | GYTFTNyGVN |
| VHmil x VK1aNQ | HCDR2 | 170 | WINPNTGEPTFDDdFKG |
| VHmil x VK1aNQ | HCDR3 | 171 | SRGKNEAwFAY |
| VHmil x VK1aNQ | LCDR1 | 172 | RSSQSLVHRNQNtYLH |
| VHmil x VK1aNQ | LCDR2 | 173 | TVSnRFS |
| VHmil x VK1aNQ | LCDR3 | 174 | SQSSHVPPT |
| SEZ6 | HCDR1 | 175 | GYTFTSYWIN |
| SEZ6 | HCDR2 | 176 | NIFPDTTTTNYNEKFKG |
| SEZ6 | HCDR3 | 177 | EYYDGTYDAMDY |
| SEZ6 | LCDR1 | 178 | KASQSVNNDVA |
| SEZ6 | LCDR2 | 179 | YASNRYT |
| SEZ6 | LCDR3 | 180 | QQDYSSPRT |
| CD56 | HCDR1 | 181 | GFTFSSFGMH |
| CD56 | HCDR2 | 182 | YISSGSFTIYYADSVKG |
| CD56 | HCDR3 | 183 | MRKGYAMDY |
| CD56 | LCDR1 | 184 | RSSQIIIHSDGNTYLE |
| CD56 | LCDR2 | 185 | KVSNRFS |
| CD56 | LCDR3 | 186 | FQGSHVPHT |
| DLL3 | HCDR1 | 187 | GYTFTNYGMN |
| DLL3 | HCDR2 | 188 | WINTYTGEPTYADDFKG |
| DLL3 | HCDR3 | 189 | IGDSSPSDY |
| DLL3 | LCDR1 | 190 | KASQSVSNDVV |
| DLL3 | LCDR2 | 179 | YASNRYT |
| DLL3 | LCDR3 | 191 | QQDYTSPWT |
| DLK1 | HCDR1 | 192 | GFNIRDTYIH |
| DLK1 | HCDR2 | 193 | RIDPPNGNLKYDPKFQG |
| DLK1 | HCDR3 | 194 | SDGYSFAY |
| DLK1 | LCDR1 | 195 | RSSKSLLHSNGNTYLY |
| DLK1 | LCDR2 | 196 | RMSNLAS |
| DLK1 | LCDR3 | 197 | MQHVEYPFT |
| B7-H3 | HCDR1 | 198 | GYTFSSYWMH |
| ABBV-155 | | | |
| B7-H3 | HCDR2 | 199 | LIHPESGSTNYNEMFKN |
| ABBV-155 | | | |
| B7-H3 | HCDR3 | 200 | GGRLYFDY |
| ABBV-155 | | | |
| B7-H3 | LCDR1 | 201 | RSSQSLVHSNRDTYLR |
| ABBV-155 | | | |
| B7-H3 | LCDR2 | 185 | KVSNRFS |
| ABBV-155 | | | |
| B7-H3 | LCDR3 | 202 | SQSTHVPYT |
| ABBV-155 | | | |
| B7-H3 | HCDR1 | 203 | GYTFTNYVMH |
| DS-5573a | | | |
| B7-H3 | HCDR2 | 204 | YINPYNDDVKYNEKFKG |
| DS-5573a | | | |
| B7-H3 | HCDR3 | 205 | WGYYGSPLYYFDY |
| DS-5573a | | | |
| B7-H3 | LCDR1 | 206 | RASSRLIYMH |
| DS-5573a | | | |
| B7-H3 | LCDR2 | 207 | ATSNLAS |
| DS-5573a | | | |
| B7-H3 | LCDR3 | 208 | QQWNSNPPT |
| DS-5573a | | | |
| IgG | HCDR1 | 209 | GDSVSSNSAAWS |
| IgG | HCDR2 | 210 | RIYYRSKWYNDYAVSVKS |
| IgG | HCDR3 | 211 | LDHRYHEDTVYPGMDV |
| IgG | LCDR1 | 212 | SGDNLPAYTVT |
| IgG | LCDR2 | 213 | DDSDRPS |
| IgG | LCDR3 | 214 | ASWDPSSGVV |

TABLE D4
Amino acid sequences of full-length mAb Ig chains

| mAb | IgG chain | SEQ ID NO | Amino acid sequence |
|---|---|---|---|
| J6M0 | Heavy chain | 57 | QVQLVQSGAEVKKPGSSVKVSCKASGGTF |
| | | | SNYWMHWVRQAPGQGLEWMGATYRGHSDT |
| | | | YYNQKFKGRVTITADKSTSTAYMELSSLR |
| | | | SEDTAVYYCARGAIYNGYDVLDNWGQGTL |
| | | | VTVSSASTKGPSVFPLAPSSKSTSGGTAA |
| | | | LGCLVKDYFPCPVTVSWNSGALTSGVHTF |
| | | | PAVLQSSGLYSLSSVVTVPSSSLGTQTYI |
| | | | CNVNHKPSNTKVDKRVEPKSCDKTHTCPP |
| | | | CPAPELLGGPSVFLFPPKPKDTLMISRTP |
| | | | EVTCVVVDVSHEDPEVKFNWYVDGVEVHN |
| | | | AKTKPREEQYNSTYRVVSVLTVLHQDWLN |
| | | | GKEYKCKVSNKALPAPIEKTISKAKGQPR |
| | | | EPQVYTLPPSREEMTKNQVSLTCLVKGFY |
| | | | PCDIAVEWESNGQPENNYKTTPPVLDSDG |
| | | | SFFLYSKLTVDKSRWQQGNVFSCSVMHEA |
| | | | LHNHYTQKSLSLSPGK |
| J6M0 | Light chain | 58 | DIQMTQSPSSLSASVGDRVTITCSASQDI |
| | | | SNYLNWYQQKPGKAPKLLIYYTSNLHSGV |
| | | | PSRFSGSGSGTDFTLTISSLQPEDFATYY |
| | | | CQQYRKLPWTFGQGTKLEIKRTVAAPSVF |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| MuMy9-6ch | Heavy chain | 59 | QVQLQQPGAEVVKPGASVKMSCKASGYTF |
| | | | TSYYIHWIKQTPGQGLEWVGVIYPGNDDT |
| | | | SYNQKFKGKATLTADKSSTTAYMQLSSLT |
| | | | SEDSAVYYCAREVRLRYFDVWGAGTTVTV |
| | | | SSASTKGPSVFPLAPSSKSTSGGTAALGC |
| | | | LVKDYFPCPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPCD |
| | | | IAVEWESNGQPENNYKTTPPVLDSDGSFF |
| | | | LYSKLTVDKSRWQQGNVFSCSVMHEALHN |
| | | | HYTQKSLSLSPGK |
| MuMy9-6ch | Light chain | 60 | NIMLTQSPSSLAVSAGEKVTMSCKSSQSV |
| | | | FFSSSQKNYLAWYQQIPGQSPKLLIYWAS |
| | | | TRESGVPDRFTGSGSGTDFTLIISSVQSE |
| | | | DLAIYYCHQYLSSRTFGGGTKLEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| gemtuzumab | Heavy chain | 61 | EVQLVQSGAEVKKPGSSVKVSCKASGYTI |
| | | | TDSNIHWVRQAPGQSLEWIGYIYPYNGGT |
| | | | DYNQKFKNRATLTVDNPTNTAYMELSSLR |
| | | | SEDTAFYYCVNGNPWLAYWGQGTLVTVSS |
| | | | ASTKGPSVFPLAPSSKSTSGGTAALGCLV |
| | | | KDYFPCPVTVSWNSGALTSGVHTFPAVLQ |
| | | | SSGLYSLSSVVTVPSSSLGTQTYICNVNH |
| | | | KPSNTKVDKRVEPKSCDKTHTCPPCPAPE |
| | | | LLGGPSVFLFPPKPKDTLMISRTPEVTCV |
| | | | VVDVSHEDPEVKFNWYVDGVEVHNAKTKP |
| | | | REEQYNSTYRVVSVLTVLHQDWLNGKEYK |
| | | | CKVSNKALPAPIEKTISKAKGQPREPQVY |
| | | | TLPPSREEMTKNQVSLTCLVKGFYPCDIA |
| | | | VEWESNGQPENNYKTTPPVLDSDGSFFLY |
| | | | SKLTVDKSRWQQGNVFSCSVMHEALHNHY |
| | | | TQKSLSLSPGK |
| gemtuzumab | Light chain | 62 | DIQLTQSPSTLSASVGDRVTITCRASESL |
| | | | DNYGIRFLTWFQQKPGKAPKLLMYAASNQ |
| | | | GSGVPSRFSGSGSGTEFTLTISSLQPDDF |
| | | | ATYYCQQTKEVPWSFGQGTKVEVKRTVAA |
| | | | PSVFIFPPSDEQLKSGTASVVCLLNNFYP |
| | | | REAKVQWKVDNALQSGNSQESVTEQDSKD |
| | | | STYSLSSTLTLSKADYEKHKVYACEVTHQ |
| | | | GLSSPVTKSFNRGEC |
| NOV169N31Q | Heavy chain | 63 | QVQLQQSGPGLVKPSQTLSLTCAISGDSV |
| | | | SSQSAAWNWIRQSPSRGLEWLGRIYYRSK |
| | | | WYNDYALSVKSRITINPDTSKNQFSLQLN |
| | | | SVTPEDTAVYYCARGEGYGREGFAIWGQG |
| | | | TLVTVSSASTKGPSVFPLAPSSKSTSGGT |
| | | | AALGCLVKDYFPCPVTVSWNSGALTSGVH |
| | | | TFPAVLQSSGLYSLSSVVTVPSSSLGTQT |
| | | | YICNVNHKPSNTKVDKRVEPKSCDKTHTC |
| | | | PPCPAPELLGGPSVFLFPPKPKDTLMISR |
| | | | TPEVTCVVVDVSHEDPEVKFNWYVDGVEV |
| | | | HNAKTKPREEQYNSTYRVVSVLTVLHQDW |
| | | | LNGKEYKCKVSNKALPAPIEKTISKAKGQ |
| | | | PREPQVYTLPPSREEMTKNQVSLTCLVKG |
| | | | FYPCDIAVEWESNGQPENNYKTTPPVLDS |
| | | | DGSFFLYSKLTVDKSRWQQGNVFSCSVMH |
| | | | EALHNHYTQKSLSLSPGK |
| NOV169N31Q | Light chain | 64 | DIQMTQSPSSLSASVGDRVTITCRASQTI |
| | | | SNTLAWYQQKPGKAPKLLIYAASNLQSGV |
| | | | PSRFSGSGSGTDFTLTISSLQPEDFATYY |
| | | | CQQYLSWFTFGQGTKVEIKRTVAAPSVFI |
| | | | FPPSDEQLKSGTASVVCLLNNFYPREAKV |
| | | | QWKVDNALQSGNSQESVTEQDSKDSTYSL |
| | | | SSTLTLSKADYEKHKVYACEVTHQGLSSP |
| | | | VTKSFNRGEC |
| trastuzumab | Heavy chain | 65 | EVQLVESGGGLVQPGGSLRLSCAASGFNI |
| | | | KDTYIHWVRQAPGKGLEWVARIYPTNGYT |
| | | | RYADSVKGRFTISADTSKNTAYLQMNSLR |
| | | | AEDTAVYYCSRWGGDGFYAMDYWGQGTLV |
| | | | TVSSASTKGPSVFPLAPSSKSTSGGTAAL |
| | | | GCLVKDYFPEPVTVSWNSGALTSGVHTFP |
| | | | AVLQSSGLYSLSSVVTVPSSSLGTQTYIC |
| | | | NVNHKPSNTKVDKKVEPPKSCDKTHTCPP |
| | | | CPAPELLGGPSVFLFPPKPKDTLMISRTP |
| | | | EVTCVVVDVSHEDPEVKFNWYVDGVEVHN |
| | | | AKTKPREEQYNSTYRVVSVLTVLHQDWLN |
| | | | GKEYKCKVSNKALPAPIEKTISKAKGQPR |
| | | | EPQVYTLPPSRDELTKNQVSLTCLVKGFY |
| | | | PSDIAVEWESNGQPENNYKTTPPVLDSDG |
| | | | SFFLYSKLTVDKSRWQQGNVFSCSVMHEA |
| | | | LHNHYTQKSLSLSPGK |
| trastuzumab | Light chain | 66 | DIQMTQSPSSLSASVGDRVTITCRASQDV |
| | | | NTAVAWYQQKPGKAPKLLIYSASFLYSGV |
| | | | PSRFSGSRSGTDFTLTISSLQPEDFATYY |
| | | | CQQHYTTPPTFGQGTKVEIKRTVAAPSVF |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| daratumumab | Heavy chain | 67 | EVQLLESGGGLVQPGGSLRLSCAVSGFTF |
| | | | NSFAMSWVRQAPGKGLEWVSAISGSGGGT |
| | | | YYADSVKGRFTISRDNSKNTLYLQMNSLR |
| | | | AEDTAVYFCAKDKILWFGEPVFDYWGQGT |
| | | | LVTVSSASTKGPSVFPLAPSSKSTSGGTA |
| | | | ALGCLVKDYFPCPVTVSWNSGALTSGVHT |
| | | | FPAVLQSSGLYSLSSVVTVPSSSLGTQTY |
| | | | ICNVNHKPSNTKVDKRVEPKSCDKTHTCP |
| | | | PCPAPELLGGPSVFLFPPKPKDTLMISRT |
| | | | PEVTCVVVD |
| | | | VSHEDPEVKFNWYVDGVEVHNAKTKPREE |
| | | | QYNSTYRVVSVLTVLHQDWLNGKEYKCKV |
| | | | SNKALPAPIEKTISKAKGQPREPQVYTLP |
| | | | PSREEMTKNQVSLTCLVKGFYPCDIAVEW |
| | | | ESNGQPENNYKTTPPVLDSDGSFFLYSKL |
| | | | TVDKSRWQQGNVFSCSVMHEALHNHYTQK |
| | | | SLSLSPGK |
| daratumumab | Light chain | 68 | EIVLTQSPATLSLSPGERATLSCRASQSV |
| | | | SSYLAWYQQKPGQAPRLLIYDASNRATGI |
| | | | PARFSGSGSGTDFTLTISSLEPEDFAVYY |
| | | | CQQRSNWPPTFGQGTKVEIKRTVAAPSVF |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| SGN-48A | Heavy chain | 69 | QVQLVQSGSELKKPGASVKVSCKASGYTF |
| | | | TDFGMNWVRQAPGQGLEWMGWINTFTGEP |
| | | | SYGNVFKGRFVFSLDTSVSTAYLQISSLK |
| | | | AEDTAVYYCARRHGNGNVFDSWGQGTLVT |
| | | | VSSASTKGPSVFPLAPSSKSTSGGTAALG |
| | | | CLVKDYFPCPVTVSWNSGALTSGVHTFPA |
| | | | VLQSSGLYSLSSVVTVPSSSLGTQTYICN |
| | | | VNHKPSNTKVDKRVEPKSCDKTHTCPPCP |
| | | | APELLGGPSVFLFPPKPKDTLMISRTPEV |
| | | | TCVVVDVSHEDPEVKFNWYVDGVEVHNAK |
| | | | TKPREEQYNSTYRVVSVLTVLHQDWLNGK |
| | | | EYKCKVSNKALPAPIEKTISKAKGQPREP |
| | | | QVYTLPPSREEMTKNQVSLTCLVKGFYPC |
| | | | DIAVEWESNGQPENNYKTTPPVLDSDGSF |
| | | | FLYSKLTVDKSRWQQGNVFSCSVMHEALH |
| | | | NHYTQKSLSLSPGK |
| SGN-48A | Light chain | 70 | EIVLTQSPDFQSVTPKEKVTITCRASQSI |
| | | | GSNIHWYQQKPDQSPKLLIKYTSESISGV |
| | | | PSRFSGSGSGTDFTLTINSLEAEDAATYY |
| | | | CQQSNSWPLTFGGGTKVEIKRTVAAPSVF |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| Polatuzimab | Heavy Chain | 88 | EVQLVESGGGLVQPGGSLRLSCAASGYTF |
| | | | SSYWIEWVRQAPGKGLEWIGEILPGGGDT |
| | | | NYNEIFKGRATFSADTSKNTAYLQMNSLR |
| | | | AEDTAVYYCTRRVPIRLDYWGQGTLVTVS |
| | | | SASTKGPSVFPLAPSSKSTSGGTAALGCL |
| | | | VKDYFPCPVTVSWNSGALTSGVHTFPAVL |
| | | | QSSGLYSLSSVVTVPSSSLGTQTYICNVN |
| | | | HKPSNTKVDKRVEPKSCDKTHTCPPCPAP |
| | | | ELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEY |
| | | | KCKVSNKALPAPIEKTISKAKGQPREPQV |
| | | | YTLPPSREEMTKNQVSLTCLVKGFYPCDI |
| | | | AVEWESNGQPENNYKTTPPVLDSDGSFFL |
| | | | YSKLTVDKSRWQQGNVFSCSVMHEALHNH |
| | | | YTQKSLSLSPGK |
| Polatuzimab | Light Chain | 89 | DIQLTQSPSSLSASVGDRVTITCKASQSV |
| | | | DYEGDSFLNWYQQKPGKAPKLLIYAASNL |
| | | | ESGVPSRFSGSGSGTDFTLTISSLQPEDF |
| | | | ATYYCQQSNEDPLTFGQGTKVEIKRTVAA |
| | | | PSVFIFPPSDEQLKSGTASVVCLLNNFYP |
| | | | REAKVQWKVDNALQSGNSQESVTEQDSKD |
| | | | STYSLSSTLTLSKADYEKHKVYACEVTHQ |
| | | | GLSSPVTKSFNrGEC |
| VHmil x VK1aNQ | Heavy Chain | 118 | QVQLQQSGSELKKPGASVKVSCKASGYTF |
| | | | TNYGVNWIKQAPGQGLQWMGWINPNTGEP |
| | | | TFDDDFKGRFAFSLDTSVSTAYLQISSLK |
| | | | ADDTAVYFCSRSRGKNEAWFAYWGQGTLV |
| | | | TVSSASTKGPSVFPLAPSSKSTSGGTAAL |
| | | | GCLVKDYFPCPVTVSWNSGALTSGVHTFP |
| | | | AVLQSSGLYSLSSVVTVPSSSLGTQTYIC |
| | | | NVNHKPSNTKVDKRVEPKSCDKTHTCPPC |
| | | | PAPELLGGPSVFLFPPKPKDTLMISRTPE |
| | | | VTCVVVDVSHEDPEVKFNWYVDGVEVHNA |
| | | | KTKPREEQYNSTYRVVSVLTVLHQDWLNG |
| | | | KEYKCKVSNKALPAPIEKTISKAKGQPRE |
| | | | PQVYTLPPSREEMTKNQVSLTCLVKGFYP |
| | | | CDIAVEWESNGQPENNYKTTPPVLDSDGS |
| | | | FFLYSKLTVDKSRWQQGNVFSCSVMHEAL |
| | | | HNHYTQKSLSLSPGK |
| | Light Chain | 216 | DIVMTQTPLSLPVTPGEPASISCRSSQSL |
| | | | VHRNQNTYLHWYLQKPGQSPQLLIYTVSN |
| | | | RFSGVPDRFSGSGSGTDFTLKISRVEAED |
| | | | VGVYFCSQSSHVPPTFGQGTKLEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| SEZ6 | Heavy chain | 217 | QVQLVQSGAEVKKPGASVKVSCKASGYTF |
| | | | TSYWINWVRQAPGQGLEWIGNIFPDTTTT |
| | | | NYNEKFKGRVTLTRDTSISTAYMELSRLR |
| | | | SDDTAVYYCAREYYDGTYDAMDYWGQGTL |
| | | | VTVSSASTKGPSVFPLAPSSKSTSGGTAA |
| | | | LGCLVKDYFPCPVTVSWNSGALTSGVHTF |
| | | | PAVLQSSGLYSLSSVVTVPSSSLGTQTYI |
| | | | CNVNHKPSNTKVDKRVEPKSCDKTHTCPP |
| | | | CPAPELLGGPSVFLFPPKPKDTLMISRTP |
| | | | EVTCVVVDVSHEDPEVKFNWYVDGVEVHN |
| | | | AKTKPREEQYNSTYRVVSVLTVLHQDWLN |
| | | | GKEYKCKVSNKALPAPIEKTISKAKGQPR |
| | | | EPQVYTLPPSREEMTKNQVSLTCLVKGFY |
| | | | PCDIAVEWESNGQPENNYKTTPPVLDSDG |
| | | | SFFLYSKLTVDKSRWQQGNVFSCSVMHEA |
| | | | LHNHYTQKSLSLSPGK |
| SEZ6 | Light chain | 218 | AIQMTQSPSSLSASVGDRVTITCKASQSV |
| | | | NNDVAWYQQKPGKAPKLLIYYASNRYTGV |
| | | | PSRFSGSGSGTDFTLTISSLQPEDFATYF |
| | | | CQQDYSSPRTFGQGTKLEIKRTVAAPSVF |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| CD56 | Heavy chain | 219 | QVQLVESGGGVVQPGRSLRLSCAASGFTF |
| (Lorovtuzumab) | | | SSFGMHWVRQAPGKGLEWVAYISSGSFTI |
| | | | YYADSVKGRFTISRDNSKNTLYLQMNSLR |
| | | | AEDTAVYYCARMRKGYAMDYWGQGTLVTV |
| | | | SSASTKGPSVFPLAPSSKSTSGGTAALGC |
| | | | LVKDYFPCPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPCD |
| | | | IAVEWESNGQPENNYKTTPPVLDSDGSFF |
| | | | LYSKLTVDKSRWQQGNVFSCSVMHEALHN |
| | | | HYTQKSLSLSPGK |
| CD56 | Light chain | 220 | DVVMTQSPLSLPVTLGQPASISCRSSQII |
| | | | IHSDGNTYLEWFQQRPGQSPRRLIYKVSN |
| | | | RFSGVPDRFSGSGSGTDFTLKISRVEAED |
| | | | VGVYYCFQGSHVPHTFGQGTKVEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| DLL3 | Heavy chain | 221 | QVQLVQSGAEVKKPGASVKVSCKASGYTF |
| (Rovalpituzumab) | | | TNYGMNWVRQAPGQGLEWMGWINTYTGEP |
| | | | TYADDFKGRVTMTTDTSTSTAYMELRSLR |
| | | | SDDTAVYYCARIGDSSPSDYWGQGTLVTV |
| | | | SSASTKGPSVFPLAPSSKSTSGGTAALGC |
| | | | LVKDYFPCPVTVSWNSGALTSGVHTFPAV |
| | | | LQSSGLYSLSSVVTVPSSSLGTQTYICNV |
| | | | NHKPSNTKVDKRVEPKSCDKTHTCPPCPA |
| | | | PELLGGPSVFLFPPKPKDTLMISRTPEVT |
| | | | CVVVDVSHEDPEVKFNWYVDGVEVHNAKT |
| | | | KPREEQYNSTYRVVSVLTVLHQDWLNGKE |
| | | | YKCKVSNKALPAPIEKTISKAKGQPREPQ |
| | | | VYTLPPSREEMTKNQVSLTCLVKGFYPCD |
| | | | IAVEWESNGQPENNYKTTPPVLDSDGSFF |
| | | | LYSKLTVDKSRWQQGNVFSCSVMHEALHN |
| | | | HYTQKSLSLSPGK |
| DLL3 | Light chain | 222 | EIVMTQSPATLSVSPGERATLSCKASQSV |
| | | | SNDVVWYQQKPGQAPRLLIYYASNRYTGI |
| | | | PARFSGSGSGTEFTLTISSLQSEDFAVYY |
| | | | CQQDYTSPWTFGQGTKLEIKRTVAAPSVF |
| | | | IFPPSDEQLKSGTASVVCLLNNFYPREAK |
| | | | VQWKVDNALQSGNSQESVTEQDSKDSTYS |
| | | | LSSTLTLSKADYEKHKVYACEVTHQGLSS |
| | | | PVTKSFNRGEC |
| DLK1 | Heavy chain | 223 | EVQLQQSGAELVKPGASVKLSCTASGFNI |
| (DI-2-14) | | | RDTYIHWVKQRPEQGLEWIGRIDPPNGNL |
| | | | KYDPKFQGKATITADTSSNTAYLQFSSLT |
| | | | SEDTAVYYCARSDGYSFAYWGQGTLVTVS |
| | | | SASTKGPSVFPLAPSSKSTSGGTAALGCL |
| | | | VKDYFPCPVTVSWNSGALTSGVHTFPAVL |
| | | | QSSGLYSLSSVVTVPSSSLGTQTYICNVN |
| | | | HKPSNTKVDKRVEPKSCDKTHTCPPCPAP |
| | | | ELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEY |
| | | | KCKVSNKALPAPIEKTISKAKGQPREPQV |
| | | | YTLPPSREEMTKNQVSLTCLVKGFYPCDI |
| | | | AVEWESNGQPENNYKTTPPVLDSDGSFFL |
| | | | YSKLTVDKSRWQQGNVFSCSVMHEALHNH |
| | | | YTQKSLSLSPGK |
| DLK1 | Light chain | 224 | DIVMTQAAPSVPVTPGESVSISCRSSKSL |
| | | | LHSNGNTYLYWFLQRPGQSPQLLIYRMSN |
| | | | LASGVPDRFSGSGSGTAFTLRISRVEAED |
| | | | VGVYYCMQHVEYPFTFGSGTKLEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| B7-H3 | Heavy chain | 225 | EVQLVQSGAEVKKPGSSVKVSCKASGYTF |
| ABBV-155 | | | SSYWMHWVRQAPGQGLEWIGLIHPESGST |
| | | | NYNEMFKNRATLTVDRSTSTAYMELSSLR |
| | | | SEDTAVYYCAGGGRLYFDYWGQGTTVTVS |
| | | | SASTKGPSVFPLAPSSKSTSGGTAALGCL |
| | | | VKDYFPCPVTVSWNSGALTSGVHTFPAVL |
| | | | QSSGLYSLSSVVTVPSSSLGTQTYICNVN |
| | | | HKPSNTKVDKRVEPKSCDKTHTCPPCPAP |
| | | | ELLGGPSVFLFPPKPKDTLMISRTPEVTC |
| | | | VVVDVSHEDPEVKFNWYVDGVEVHNAKTK |
| | | | PREEQYNSTYRVVSVLTVLHQDWLNGKEY |
| | | | KCKVSNKALPAPIEKTISKAKGQPREPQV |
| | | | YTLPPSREEMTKNQVSLTCLVKGFYPCDI |
| | | | AVEWESNGQPENNYKTTPPVLDSDGSFFL |
| | | | YSKLTVDKSRWQQGNVFSCSVMHEALHNH |
| | | | YTQKSLSLSPGK |
| B7-H3 | Light chain | 226 | DIVMTQSPLSLPVTPGEPASISCRSSQSL |
| ABBV-155 | | | VHSNRDTYLRWYLQKPGQSPQLLIYKVSN |
| | | | RFSGVPDRFSGSGSGTDFTLKISRVEAED |
| | | | VGVYYCSQSTHVPYTFGGGTKVEIKRTVA |
| | | | APSVFIFPPSDEQLKSGTASVVCLLNNFY |
| | | | PREAKVQWKVDNALQSGNSQESVTEQDSK |
| | | | DSTYSLSSTLTLSKADYEKHKVYACEVTH |
| | | | QGLSSPVTKSFNRGEC |
| B7-H3 | Heavy Chain | 227 | QVQLVQSGAEVKKPGSSVKVSCKASGYTF |
| DS-5573a | | | TNYVMHWVRQAPGQGLEWMGYINPYNDDV |
| | | | KYNEKFKGRVTITADESTSTAYMELSSLR |
| | | | SEDTAVYYCARWGYYGSPLYYFDYWGQGT |
| | | | LVTVSSASTKGPSVFPLAPSSKSTSGGTA |
| | | | ALGCLVKDYFPCPVTVSWNSGALTSGVHT |
| | | | FPAVLQSSGLYSLSSVVTVPSSSLGTQTY |
| | | | ICNVNHKPSNTKVDKRVEPKSCDKTHTCP |
| | | | PCPAPELLGGPSVFLFPPKPKDTLMISRT |
| | | | PEVTCVVVDVSHEDPEVKFNWYVDGVEVH |
| | | | NAKTKPREEQYNSTYRVVSVLTVLHQDWL |
| | | | NGKEYKCKVSNKALPAPIEKTISKAKGQP |
| | | | REPQVYTLPPSREEMTKNQVSLTCLVKGF |
| | | | YPCDIAVEWESNGQPENNYKTTPPVLDSD |
| | | | GSFFLYSKLTVDKSRWQQGNVFSCSVMHE |
| | | | ALHNHYTQKSLSLSPGK |
| B7-H3 | Light Chain | 228 | EIVLTQSPATLSLSPGERATLSCRASSRL |
| DS-5573a | | | IYMHWYQQKPGQAPRPLIYATSNLASGIP |
| | | | ARFSGSGSGTDFTLTISSLEPEDFAVYYC |
| | | | QQWNSNPPTFGQGTKVEIKRTVAAPSVFI |
| | | | FPPSDEQLKSGTASVVCLLNNFYPREAKV |
| | | | QWKVDNALQSGNSQESVTEQDSKDSTYSL |
| | | | SSTLTLSKADYEKHKVYACEVTHQGLSSP |
| | | | VTKSFNRGEC |
| IgG | Heavy Chain | 229 | QVQLQQSGPGLVKPSQTLSLTCAISGDSV |
| | | | SSNSAAWSWIRQSPGRGLEWLGRIYYRSK |
| | | | WYNDYAVSVKSRITINPDTSKNQFSLQLN |
| | | | SVTPEDTAVYYCARLDHRYHEDTVYPGMD |
| | | | VWGQGTLVTVSSASTKGPSVFPLAPSSKS |
| | | | TSGGTAALGCLVKDYFPEPVTVSWNSGAL |
| | | | TSGVHTFPAVLQSSGLYSLSSVVTVPSSS |
| | | | LGTQTYICNVNHKPSNTKVDKRVEPKSCD |
| | | | KTHTCPPCPAPELLGGPSVFLFPPKPKDT |
| | | | LMISRTPEVTCVVVDVSHEDPEVKFNWYV |
| | | | DGVEVHNAKTKPREEQYNSTYRVVSVLTV |
| | | | LHQDWLNGKEYKCKVSNKALPAPIEKTIS |
| | | | KAKGQPREPQVYTLPPSREEMTKNQVSLT |
| | | | CLVKGFYPSDIAVEWESNGQPENNYKTTP |
| | | | PVLDSDGSFFLYSKLTVDKSRWQQGNVFS |
| | | | CSVMHEALHNHYTQKSLSLSPG |
| IgG | Light Chain | 230 | DIELTQPPSVSVAPGQTARISCSGDNLPA |
| | | | YTVTWYQQKPGQAPVLVIYDDSDRPSGIP |
| | | | ERFSGSNSGNTATLTISGTQAEDEADYYC |
| | | | ASWDPSSGVVFGGGTKLTVLGQPKAAPSV |
| | | | TLFPPSSEELQANKATLVCLISDFYPGAV |
| | | | TVAWKADSSPVKAGVETTTPSKQSNNKYA |
| | | | ASSYLSLTPEQWKSHRSYSCQVTHEGSTV |
| | | | EKTVAPTECS |

TABLE D5
Amino acid sequences of full-length mAb Ig chains

| | | | Light Chain |
|---|---|---|---|
| Antibody | | Heavy Chain | (CysMab) |
| Target/Code | Antibody Name | (CysMab) Sequence | Sequence |
| EGFR1 | Cetuximab | QVQLKQSGPGLVQPSQS | DILLTQSPVILSVS |
| | | LSITCTVSGFSLTNYGV | PGERVSFSCRASQS |
| | | HWVRQSPGKGLEWLGVI | IGTNIHWYQQRTNG |
| | | WSGGNTDYNTPFTSRLS | SPRLLIKYASESIS |
| | | INKDNSKSQVFFKMNSL | GIPSRFSGSGSGTD |
| | | QSNDTAIYYCARALTYY | FTLSINSVESEDIA |
| | | DYEFAYWGQGTLVTVSA | DYYCQQNNNWPTTF |
| | | ASTKGPSVFPLAPSSKS | GAGTKLELKRTVAA |
| | | TSGGTAALGCLVKDYFP | PSVFIFPPSDEQLK |
| | | CPVTVSWNSGALTSGVH | SGTASVVCLLNNFY |
| | | TFPAVLQSSGLYSLSSV | PREAKVQWKVDNAL |
| | | VTVPSSSLGTQTYICNV | QSGNSQESVTEQDS |
| | | NHKPSNTKVDKRVEPKS | KDSTYSLSSTLTLS |
| | | CDKTHTCPPCPAPELLG | KADYEKHKVYACEV |
| | | GPSVFLFPPKPKDTLMI | THQGLSSPVTKSFN |
| | | SRTPEVTCVVVDVSHED | RGEC (SEQ ID |
| | | PEVKFNWYVDGVEVHNA | NO: 93) |
| | | KTKPREEQYNSTYRVVS | |
| | | VLTVLHQDWLNGKEYKC | |
| | | KVSNKALPAPIEKTISK | |
| | | AKGQPREPQVYTLPPSR | |
| | | EEMTKNQVSLTCLVKGF | |
| | | YPCDIAVEWESNGQPEN | |
| | | NYKTTPPVLDSDGSFFL | |
| | | YSKLTVDKSRWQQGNVF | |
| | | SCSVMHEALHNHYTQKS | |
| | | LSLSPGK (SEQ ID | |
| | | NO: 92) | |
| TFRC | CD71 (CX-2029) | QVQLVQSGAEVKKPGAS | DIVLTQSPLSLAVS |
| | | VKMSCKASGYTFTSYWM | LGQPAIISCKASQS |
| | | HWVRQAPGQGLEWIGAI | VSFAGTSLMHWYHQ |
| | | YPGNSETGYAQKFQGRA | KPGQQPRLLIYRAS |
| | | TLTADTSTSTAYMELSS | NLEAGVPDRFSGSG |
| | | LRSEDTAVYYCTRENWD | SKTDFTLTISPVEA |
| | | PGFAFWGQGTLITVSSA | EDAATYYCQQSREY |
| | | STKGPSVFPLAPSSKST | PYTFGGGTKLEIKR |
| | | SGGTAALGCLVKDYFPC | TVAAPSVFIFPPSD |
| | | PVTVSWNSGALTSGVHT | EQLKSGTASVVCLL |
| | | FPAVLQSSGLYSLSSVV | NNFYPREAKVQWKV |
| | | TVPSSSLGTQTYICNVN | DNALQSGNSQESVT |
| | | HKPSNTKVDKRVEPKSC | EQDSKDSTYSLSST |
| | | DKTHTCPPCPAPELLGG | LTLSKADYEKHKVY |
| | | PSVFLFPPKPKDTLMIS | ACEVTHQGLSSPVT |
| | | RTPEVTCVVVDVSHEDP | KSFNRGEC (SEQ |
| | | EVKFNWYVDGVEVHNAK | ID NO: 95) |
| | | TKPREEQYNSTYRVVSV | |
| | | LTVLHQDWLNGKEYKCK | |
| | | VSNKALPAPIEKTISKA | |
| | | KGQPREPQVYTLPPSRE | |
| | | EMTKNQVSLTCLVKGFY | |
| | | PCDIAVEWESNGQPENN | |
| | | YKTTPPVLDSDGSFFLY | |
| | | SKLTVDKSRWQQGNVFS | |
| | | CSVMHEALHNHYTQKSL | |
| | | SLSPGK (SEQ ID | |
| | | NO: 94) | |
| EPCAM | Oportuzumab* | EVQLVQSGPGLVQPGGS | DIQMTQSPSSLSAS |
| | *Variable heavy | VRISCAASGYTFTNYGM | VGDRVTITCRSTKS |
| | chain and variable | NWVKQAPGKGLEWMGWI | LLHSNGITYLYWYQ |
| | light chain | NTYTGESTYADSFKGRF | QKPGKAPKLLIYQM |
| | sequence of | TFSLDTSASAAYLQINS | SNLASGVPSRFSSS |
| | oportuzumab | LRAEDTAVYYCARFAIK | GSGTDFTLTISSLQ |
| | monatox were | GDYWGQGTLLTVSSAST | PEDFATYYCAQNLE |
| | combined with | KGPSVFPLAPSSKSTSG | IPRTFGQGTKVELK |
| | human heavy chain | GTAALGCLVKDYFPCPV | RTVAAPSVFIFPPS |
| | constant region and | TVSWNSGALTSGVHTFP | DEQLKSGTASVVCL |
| | light chain constant | AVLQSSGLYSLSSVVTV | LNNFYPREAKVQWK |
| | region to produce | PSSSLGTQTYICNVNHK | VDNALQSGNSQESV |
| | full IgG antibody | PSNTKVDKRVEPKSCDK | TEQDSKDSTYSLSS |
| | sequence | THTCPPCPAPELLGGPS | TLTLSKADYEKHKV |
| | | VFLFPPKPKDTLMISRT | YACEVTHQGLSSPV |
| | | PEVTCVVVDVSHEDPEV | TKSFNRGEC (SEQ |
| | | KFNWYVDGVEVHNAKTK | ID NO: 97) |
| | | PREEQYNSTYRVVSVLT | |
| | | VLHQDWLNGKEYKCKVS | |
| | | NKALPAPIEKTISKAKG | |
| | | QPREPQVYTLPPSREEM | |
| | | TKNQVSLTCLVKGFYPC | |
| | | DIAVEWESNGQPENNYK | |
| | | TTPPVLDSDGSFFLYSK | |
| | | LTVDKSRWQQGNVFSCS | |
| | | VMHEALHNHYTQKSLSL | |
| | | SPGK (SEQ ID | |
| | | NO: 96) | |
| FOLR1 | Mirvetuximab | QVQLVQSGAEVVKPGAS | DIVLTQSPLSLAVS |
| | | VKISCKASGYTFTGYFM | LGQPAIISCKASQS |
| | | NWVKQSPGQSLEWIGRI | VSFAGTSLMHWYHQ |
| | | HPYDGDTFYNQKFQGKA | KPGQQPRLLIYRAS |
| | | TLTVDKSSNTAHMELLS | NLEAGVPDRFSGSG |
| | | LTSEDFAVYYCTRYDGS | SKTDFTLTISPVEA |
| | | RAMDYWGQGTTVTVSSA | EDAATYYCQQSREY |
| | | STKGPSVFPLAPSSKST | PYTFGGGTKLEIKR |
| | | SGGTAALGCLVKDYFPC | TVAAPSVFIFPPSD |
| | | PVTVSWNSGALTSGVHT | EQLKSGTASVVCLL |
| | | FPAVLQSSGLYSLSSVV | NNFYPREAKVQWKV |
| | | TVPSSSLGTQTYICNVN | DNALQSGNSQESVT |
| | | HKPSNTKVDKRVEPKSC | EQDSKDSTYSLSST |
| | | DKTHTCPPCPAPELLGG | LTLSKADYEKHKVY |
| | | PSVFLFPPKPKDTLMIS | ACEVTHQGLSSPVT |
| | | RTPEVTCVVVDVSHEDP | KSFNRGEC (SEQ |
| | | EVKFNWYVDGVEVHNAK | ID NO: 99) |
| | | TKPREEQYNSTYRVVSV | |
| | | LTVLHQDWLNGKEYKCK | |
| | | VSNKALPAPIEKTISKA | |
| | | KGQPREPQVYTLPPSRE | |
| | | EMTKNQVSLTCLVKGFY | |
| | | PCDIAVEWESNGQPENN | |
| | | YKTTPPVLDSDGSFFLY | |
| | | SKLTVDKSRWQQGNVFS | |
| | | CSVMHEALHNHYTQKSL | |
| | | SLSPGK (SEQ ID | |
| | | NO: 98) | |
| ENPP3 | ENPP3 (AGS16-7.8) | QVQLQESGPGLVKPSQT | EIVLTQSPDFQSVT |
| | | LSLTCTVSGGSISSGGY | PKEKVTITCRASQS |
| | | YWSWIRQHPGKGLEWIG | IGISLHWYQQKPDQ |
| | | IIYYSGSTYYNPSLKSR | SPKLLIKYASQSFS |
| | | VTISVDTSKNQFSLKLN | GVPSRFSGSGSGTD |
| | | SVTAADTAVFYCARVAI | FTLTINSLEAEDAA |
| | | VTTIPGGMDVWGQGTTV | TYYCHQSRSFPWTF |
| | | TVSSASTKGPSVFPLAP | GQGTKVEIKRTVAA |
| | | SSKSTSGGTAALGCLVK | PSVFIFPPSDEQLK |
| | | DYFPCPVTVSWNSGALT | SGTASVVCLLNNFY |
| | | SGVHTFPAVLQSSGLYS | PREAKVQWKVDNAL |
| | | LSSVVTVPSSSLGTQTY | QSGNSQESVTEQDS |
| | | ICNVNHKPSNTKVDKRV | KDSTYSLSSTLTLS |
| | | EPKSCDKTHTCPPCPAP | KADYEKHKVYACEV |
| | | ELLGGPSVFLFPPKPKD | THQGLSSPVTKSFN |
| | | TLMISRTPEVTCVVVDV | RGEC (SEQ ID |
| | | SHEDPEVKFNWYVDGVE | NO: 101) |
| | | VHNAKTKPREEQYNSTY | |
| | | RVVSVLTVLHQDWLNGK | |
| | | EYKCKVSNKALPAPIEK | |
| | | TISKAKGQPREPQVYTL | |
| | | PPSREEMTKNQVSLTCL | |
| | | VKGFYPCDIAVEWESNG | |
| | | QPENNYKTTPPVLDSDG | |
| | | SFFLYSKLTVDKSRWQQ | |
| | | GNVFSCSVMHEALHNHY | |
| | | TQKSLSLSPGK (SEQ | |
| | | ID NO: 100) | |
| MET | Telisotuzumab | QVQLVQSGAEVKKPGAS | DIVMTQSPDSLAVS |
| | | VKVSCKASGYIFTAYTM | LGERATINCKSSES |
| | | HWVRQAPGQGLEWMGWI | VDSYANSFLHWYQQ |
| | | KPNNGLANYAQKFQGRV | KPGQPPKLLIYRAS |
| | | TMTRDTSISTAYMELSR | TRESGVPDRFSGSG |
| | | LRSDDTAVYYCARSEIT | SGTDFTLTISSLQA |
| | | TEFDYWGQGTLVTVSSA | EDVAVYYCQQSKED |
| | | STKGPSVFPLAPSSKST | PLTFGGGTKVEIKR |
| | | SGGTAALGCLVKDYFPC | TVAAPSVFIFPPSD |
| | | PVTVSWNSGALTSGVHT | EQLKSGTASVVCLL |
| | | FPAVLQSSGLYSLSSVV | NNFYPREAKVQWKV |
| | | TVPSSSLGTQTYICNVN | DNALQSGNSQESVT |
| | | HKPSNTKVDKRVEPKSC | EQDSKDSTYSLSST |
| | | DKTHTCPPCPAPELLGG | LTLSKADYEKHKVY |
| | | PSVFLFPPKPKDTLMIS | ACEVTHQGLSSPVT |
| | | RTPEVTCVVVDVSHEDP | KSFNRGEC (SEQ |
| | | EVKFNWYVDGVEVHNAK | ID NO: 103) |
| | | TKPREEQYNSTYRVVSV | |
| | | LTVLHQDWLNGKEYKCK | |
| | | VSNKALPAPIEKTISKA | |
| | | KGQPREPQVYTLPPSRE | |
| | | EMTKNQVSLTCLVKGFY | |
| | | PCDIAVEWESNGQPENN | |
| | | YKTTPPVLDSDGSFFLY | |
| | | SKLTVDKSRWQQGNVFS | |
| | | CSVMHEALHNHYTQKSL | |
| | | SLSPGK (SEQ ID | |
| | | NO: 102) | |
| AXL | Enapotamab | EVQLLESGGGLVQPGGS | EIVLTQSPGTLSLS |
| | | LRLSCAASGFTFSSYAM | PGERATLSCRASQS |
| | | NWVRQAPGKGLEWVSTT | VSSSYLAWYQQKPG |
| | | SGSGASTYYADSVKGRF | QAPRLLIYGASSRA |
| | | TISRDNSKNTLYLQMNS | TGIPDRFSGSGSGT |
| | | LRAEDTAVYYCAKIWIA | DFTLTISRLEPEDF |
| | | FDIWGQGTMVTVSSAST | AVYYCQQYGSSPYT |
| | | KGPSVFPLAPSSKSTSG | FGQGTKLEIKRTVA |
| | | GTAALGCLVKDYFPCPV | APSVFIFPPSDEQL |
| | | TVSWNSGALTSGVHTFP | KSGTASVVCLLNNF |
| | | AVLQSSGLYSLSSVVTV | YPREAKVQWKVDNA |
| | | PSSSLGTQTYICNVNHK | LQSGNSQESVTEQD |
| | | PSNTKVDKRVEPKSCDK | SKDSTYSLSSTLTL |
| | | THTCPPCPAPELLGGPS | SKADYEKHKVYACE |
| | | VFLFPPKPKDTLMISRT | VTHQGLSSPVTKSF |
| | | PEVTCVVVDVSHEDPEV | NRGEC (SEQ ID |
| | | KFNWYVDGVEVHNAKTK | NO: 105) |
| | | PREEQYNSTYRVVSVLT | |
| | | VLHQDWLNGKEYKCKVS | |
| | | NKALPAPIEKTISKAKG | |
| | | QPREPQVYTLPPSREEM | |
| | | TKNQVSLTCLVKGFYPC | |
| | | DIAVEWESNGQPENNYK | |
| | | TTPPVLDSDGSFFLYSK | |
| | | LTVDKSRWQQGNVFSCS | |
| | | VMHEALHNHYTQKSLSL | |
| | | SPGK (SEQ ID | |
| | | NO: 104) | |
| SLC34A2 | Lifastuzumab | EVQLVESGGGLVQPGGS | DIQMTQSPSSLSAS |
| | | LRLSCAASGFSFSDFAM | VGDRVTITCRSSET |
| | | SWVRQAPGKGLEWVATI | LVHSSGNTYLEWYQ |
| | | GRVAFHTYYPDSMKGRF | QKPGKAPKLLIYRV |
| | | TISRDNSKNTLYLQMNS | SNRFSGVPSRFSGS |
| | | LRAEDTAVYYCARHRGF | GSGTDFTLTISSLQ |
| | | DVGHFDFWGQGTLVTVS | PEDFATYYCFQGSF |
| | | SASTKGPSVFPLAPSSK | NPLTFGQGTKVEIK |
| | | STSGGTAALGCLVKDYF | RTVAAPSVFIFPPS |
| | | PCPVTVSWNSGALTSGV | DEQLKSGTASVVCL |
| | | HTFPAVLQSSGLYSLSS | LNNFYPREAKVQWK |
| | | VVTVPSSSLGTQTYICN | VDNALQSGNSQESV |
| | | VNHKPSNTKVDKRVEPK | TEQDSKDSTYSLSS |
| | | SCDKTHTCPPCPAPELL | TLTLSKADYEKHKV |
| | | GGPSVFLFPPKPKDTLM | YACEVTHQGLSSPV |
| | | ISRTPEVTCVVVDVSHE | TKSFNRGEC (SEQ |
| | | DPEVKFNWYVDGVEVHN | ID NO: 107) |
| | | AKTKPREEQYNSTYRVV | |
| | | SVLTVLHQDWLNGKEYK | |
| | | CKVSNKALPAPIEKTIS | |
| | | KAKGQPREPQVYTLPPS | |
| | | REEMTKNQVSLTCLVKG | |
| | | FYPCDIAVEWESNGQPE | |
| | | NNYKTTPPVLDSDGSFF | |
| | | LYSKLTVDKSRWQQGNV | |
| | | FSCSVMHEALHNHYTQK | |
| | | SLSLSPGK (SEQ ID | |
| | | NO: 106) | |
| NECTIN4 | Enfortumab | EVQLVESGGGLVQPGGS | DIQMTQSPSSVSAS |
| | | LRLSCAASGFTFSSYNM | VGDRVTITCRASQG |
| | | NWVRQAPGKGLEWVSYI | ISGWLAWYQQKPGK |
| | | SSSSSTIYYADSVKGRF | APKFLIYAASTLQS |
| | | TISRDNAKNSLSLQMNS | GVPSRFSGSGSGTD |
| | | LRDEDTAVYYCARAYYY | FTLTISSLQPEDFA |
| | | GMDVWGQGTTVTVSSAS | TYYCQQANSFPPTF |
| | | TKGPSVFPLAPSSKSTS | GGGTKVEIKRTVAA |
| | | GGTAALGCLVKDYFPCP | PSVFIFPPSDEQLK |
| | | VTVSWNSGALTSGVHTF | SGTASVVCLLNNFY |
| | | PAVLQSSGLYSLSSVVT | PREAKVQWKVDNAL |
| | | VPSSSLGTQTYICNVNH | QSGNSQESVTEQDS |
| | | KPSNTKVDKRVEPKSCD | KDSTYSLSSTLTLS |
| | | KTHTCPPCPAPELLGGP | KADYEKHKVYACEV |
| | | SVFLFPPKPKDTLMISR | THQGLSSPVTKSFN |
| | | TPEVTCVVVDVSHEDPE | RGEC (SEQ ID |
| | | VKFNWYVDGVEVHNAKT | NO: 109) |
| | | KPREEQYNSTYRVVSVL | |
| | | TVLHQDWLNGKEYKCKV | |
| | | SNKALPAPIEKTISKAK | |
| | | GQPREPQVYTLPPSREE | |
| | | MTKNQVSLTCLVKGFYP | |
| | | CDIAVEWESNGQPENNY | |
| | | KTTPPVLDSDGSFFLYS | |
| | | KLTVDKSRWQQGNVFSC | |
| | | SVMHEALHNHYTQKSLS | |
| | | LSPGK (SEQ ID | |
| | | NO: 108) | |
| TACSTD2 | Sacituzumab | QVQLQQSGSELKKPGAS | DIQLTQSPSSLSAS |
| | | VKVSCKASGYTFTNYGM | VGDRVSITCKASQD |
| | | NWVKQAPGQGLKWMGWI | VSIAVAWYQQKPGK |
| | | NTYTGEPTYTDDFKGRF | APKLLIYSASYRYT |
| | | AFSLDTSVSTAYLQISS | GVPDRFSGSGSGTD |
| | | LKADDTAVYFCARGGFG | FTLTISSLQPEDFA |
| | | SSYWYFDVWGQGSLVTV | VYYCQQHYITPLTF |
| | | SSASTKGPSVFPLAPSS | GAGTKVEIKRTVAA |
| | | KSTSGGTAALGCLVKDY | PSVFIFPPSDEQLK |
| | | FPCPVTVSWNSGALTSG | SGTASVVCLLNNFY |
| | | VHTFPAVLQSSGLYSLS | PREAKVQWKVDNAL |
| | | SVVTVPSSSLGTQTYIC | QSGNSQESVTEQDS |
| | | NVNHKPSNTKVDKRVEP | KDSTYSLSSTLTLS |
| | | KSCDKTHTCPPCPAPEL | KADYEKHKVYACEV |
| | | LGGPSVFLFPPKPKDTL | THQGLSSPVTKSFN |
| | | MISRTPEVTCVVVDVSH | RGEC (SEQ ID |
| | | EDPEVKFNWYVDGVEVH | NO: 111) |
| | | NAKTKPREEQYNSTYRV | |
| | | VSVLTVLHQDWLNGKEY | |
| | | KCKVSNKALPAPIEKTI | |
| | | SKAKGQPREPQVYTLPP | |
| | | SREEMTKNQVSLTCLVK | |
| | | GFYPCDIAVEWESNGQP | |
| | | ENNYKTTPPVLDSDGSF | |
| | | FLYSKLTVDKSRWQQGN | |
| | | VFSCSVMHEALHNHYTQ | |
| | | KSLSLSPGK (SEQ ID | |
| | | NO: 110) | |
| SLC39A6 | Ladiratuzumab | QVQLVQSGAEVKKPGAS | DVVMTQSPLSLPVT |
| | | VKVSCKASGLTIEDYYM | LGQPASISCRSSQS |
| | | HWVRQAPGQGLEWMGWI | LLHSSGNTYLEWYQ |
| | | DPENGDTEYGPKFQGRV | QRPGQSPRPLIYKI |
| | | TMTRDTSINTAYMELSR | STRFSGVPDRFSGS |
| | | LRSDDTAVYYCAVHNAH | GSGTDFTLKISRVE |
| | | YGTWFAYWGQGTLVTVS | AEDVGVYYCFQGSH |
| | | SASTKGPSVFPLAPSSK | VPYTFGGGTKVEIK |
| | | STSGGTAALGCLVKDYF | RTVAAPSVFIFPPS |
| | | PCPVTVSWNSGALTSGV | DEQLKSGTASVVCL |
| | | HTFPAVLQSSGLYSLSS | LNNFYPREAKVQWK |
| | | VVTVPSSSLGTQTYICN | VDNALQSGNSQESV |
| | | VNHKPSNTKVDKRVEPK | TEQDSKDSTYSLSS |
| | | SCDKTHTCPPCPAPELL | TLTLSKADYEKHKV |
| | | GGPSVFLFPPKPKDTLM | YACEVTHQGLSSPV |
| | | ISRTPEVTCVVVDVSHE | TKSFNRGEC (SEQ |
| | | DPEVKFNWYVDGVEVHN | ID NO: 113) |
| | | AKTKPREEQYNSTYRVV | |
| | | SVLTVLHQDWLNGKEYK | |
| | | CKVSNKALPAPIEKTIS | |
| | | KAKGQPREPQVYTLPPS | |
| | | REEMTKNQVSLTCLVKG | |
| | | FYPCDIAVEWESNGQPE | |
| | | NNYKTTPPVLDSDGSFF | |
| | | LYSKLTVDKSRWQQGNV | |
| | | FSCSVMHEALHNHYTQK | |
| | | SLSLSPGK (SEQ ID | |
| | | NO: 112) | |
| GPNMB | Glembatumumab | QVQLQESGPGLVKPSQT | EIVMTQSPATLSVS |
| | | LSLTCTVSGGSISSFNY | PGERATLSCRASQS |
| | | YWSWIRHHPGKGLEWIG | VDNNLVWYQQKPGQ |
| | | YIYYSGSTYSNPSLKSR | APRLLIYGASTRAT |
| | | VTISVDTSKNQFSLTLS | GIPARFSGSGSGTE |
| | | SVTAADTAVYYCARGYN | FTLTISSLQSEDFA |
| | | WNYFDYWGQGTLVTVSS | VYYCQQYNNWPPWT |
| | | ASTKGPSVFPLAPSSKS | FGQGTKVEIKRTVA |
| | | TSGGTAALGCLVKDYFP | APSVFIFPPSDEQL |
| | | CPVTVSWNSGALTSGVH | KSGTASVVCLLNNF |
| | | TFPAVLQSSGLYSLSSV | YPREAKVQWKVDNA |
| | | VTVPSSSLGTQTYICNV | LQSGNSQESVTEQD |
| | | NHKPSNTKVDKRVEPKS | SKDSTYSLSSTLTL |
| | | CDKTHTCPPCPAPELLG | SKADYEKHKVYACE |
| | | GPSVFLFPPKPKDTLMI | VTHQGLSSPVTKSF |
| | | SRTPEVTCVVVDVSHED | NRGEC (SEQ ID |
| | | PEVKFNWYVDGVEVHNA | NO: 115) |
| | | KTKPREEQYNSTYRVVS | |
| | | VLTVLHQDWLNGKEYKC | |
| | | KVSNKALPAPIEKTISK | |
| | | AKGQPREPQVYTLPPSR | |
| | | EEMTKNQVSLTCLVKGF | |
| | | YPCDIAVEWESNGQPEN | |
| | | NYKTTPPVLDSDGSFFL | |
| | | YSKLTVDKSRWQQGNVF | |
| | | SCSVMHEALHNHYTQKS | |
| | | LSLSPGK (SEQ ID | |
| | | NO: 114) | |
| MSLN | Anetumab | QVELVQSGAEVKKPGES | DIALTQPASVSGSP |
| | | LKISCKGSGYSFTSYWI | GQSITISCTGTSSD |
| | | GWVRQAPGKGLEWMGII | IGGYNSVSWYQQHP |
| | | DPGDSRTRYSPSFQGQV | GKAPKLMIYGVNNR |
| | | TISADKSISTAYLQWSS | PSGVSNRFSGSKSG |
| | | LKASDTAMYYCARGQLY | NTASLTISGLQAED |
| | | GGTYMDGWGQGTLVTVS | EADYYCSSYDIESA |
| | | SASTKGPSVFPLAPSSK | TPVFGGGTKLTVLG |
| | | STSGGTAALGCLVKDYF | QPKAAPSVTLFPPS |
| | | PCPVTVSWNSGALTSGV | SEELQANKATLVCL |
| | | HTFPAVLQSSGLYSLSS | ISDFYPGAVTVAWK |
| | | VVTVPSSSLGTQTYICN | ADSSPVKAGVETTT |
| | | VNHKPSNTKVDKRVEPK | PSKQSNNKYAASSY |
| | | SCDKTHTCPPCPAPELL | LSLTPEQWKSHRSY |
| | | GGPSVFLFPPKPKDTLM | SCQVTHEGSTVEKT |
| | | ISRTPEVTCVVVDVSHE | VAPTECS(SEQ |
| | | DPEVKFNWYVDGVEVHN | ID NO: 117) |
| | | AKTKPREEQYNSTYRVV | |
| | | SVLTVLHQDWLNGKEYK | |
| | | CKVSNKALPAPIEKTIS | |
| | | KAKGQPREPQVYTLPPS | |
| | | REEMTKNQVSLTCLVKG | |
| | | FYPCDIAVEWESNGQPE | |
| | | NNYKTTPPVLDSDGSFF | |
| | | LYSKLTVDKSRWQQGNV | |
| | | FSCSVMHEALHNHYTQK | |
| | | SLSLSPGK (SEQ ID | |
| | | NO: 116) | |
| CD74 | Milatuzumab | QVQLQQSGSELKKPGAS | DIQLTQSPLSLPVT |
| | | VKVSCKASGYTFTNYGV | LGQPASISCRSSQS |
| | | NWIKQAPGQGLQWMGWI | LVHRNGNTYLHWFQ |
| | | NPNTGEPTFDDDFKGRF | QRPGQSPRLLIYTV |
| | | AFSLDTSVSTAYLQISS | SNRFSGVPDRFSGS |
| | | LKADDTAVYFCSRSRGK | GSGTDFTLKISRVE |
| | | NEAWFAYWGQGTLVTVS | AEDVGVYFCSQSSH |
| | | SASTKGPSVFPLAPSSK | VPPTFGAGTRLEIK |
| | | STSGGTAALGCLVKDYF | RTVAAPSVFIFPPS |
| | | PCPVTVSWNSGALTSGV | DEQLKSGTASVVCL |
| | | HTFPAVLQSSGLYSLSS | LNNFYPREAKVQWK |
| | | VVTVPSSSLGTQTYICN | VDNALQSGNSQESV |
| | | VNHKPSNTKVDKRVEPK | TEQDSKDSTYSLSS |
| | | SCDKTHTCPPCPAPELL | TLTLSKADYEKHKV |
| | | GGPSVFLFPPKPKDTLM | YACEVTHQGLSSPV |
| | | ISRTPEVTCVVVDVSHE | TKSFNRGEC (SEQ |
| | | DPEVKFNWYVDGVEVHN | ID NO: 119) |
| | | AKTKPREEQYNSTYRVV | |
| | | SVLTVLHQDWLNGKEYK | |
| | | CKVSNKALPAPIEKTIS | |
| | | KAKGQPREPQVYTLPPS | |
| | | REEMTKNQVSLTCLVKG | |
| | | FYPCDIAVEWESNGQPE | |
| | | NNYKTTPPVLDSDGSFF | |
| | | LYSKLTVDKSRWQQGNV | |
| | | FSCSVMHEALHNHYTQK | |
| | | SLSLSPGK (SEQ ID | |
| | | NO: 118) | |
| F3 | Tisotumab | EVQLLESGGGLVQPGGS | DIQMTQSPPSLSAS |
| | | LRLSCAASGFTFSNYAM | AGDRVTITCRASQG |
| | | SWVRQAPGKGLEWVSSI | ISSRLAWYQQKPEK |
| | | SGSGDYTYYTDSVKGRF | APKSLIYAASSLQS |
| | | TISRDNSKNTLYLQMNS | GVPSRFSGSGSGTD |
| | | LRAEDTAVYYCARSPWG | FTLTISSLQPEDFA |
| | | YYLDSWGQGTLVTVSSA | TYYCQQYNSYPYTF |
| | | STKGPSVFPLAPSSKST | GQGTKLEIKRTVAA |
| | | SGGTAALGCLVKDYFPC | PSVFIFPPSDEQLK |
| | | PVTVSWNSGALTSGVHT | SGTASVVCLLNNFY |
| | | FPAVLQSSGLYSLSSVV | PREAKVQWKVDNAL |
| | | TVPSSSLGTQTYICNVN | QSGNSQESVTEQDS |
| | | HKPSNTKVDKRVEPKSC | KDSTYSLSSTLTLS |
| | | DKTHTCPPCPAPELLGG | KADYEKHKVYACEV |
| | | PSVFLFPPKPKDTLMIS | THQGLSSPVTKSFN |
| | | RTPEVTCVVVDVSHEDP | RGEC (SEQ ID |
| | | EVKFNWYVDGVEVHNAK | NO: 121) |
| | | TKPREEQYNSTYRVVSV | |
| | | LTVLHQDWLNGKEYKCK | |
| | | VSNKALPAPIEKTISKA | |
| | | KGQPREPQVYTLPPSRE | |
| | | EMTKNQVSLTCLVKGFY | |
| | | PCDIAVEWESNGQPENN | |
| | | YKTTPPVLDSDGSFFLY | |
| | | SKLTVDKSRWQQGNVFS | |
| | | CSVMHEALHNHYTQKSL | |
| | | SLSPGK (SEQ ID | |
| | | NO: 120) | |
| MUC16 | Sofituzumab | EVQLVESGGGLVQPGGS | DIQMTQSPSSLSAS |
| | | LRLSCAASGYSITNDYA | VGDRVTITCKASDL |
| | | WNWVRQAPGKGLEWVGY | IHNWLAWYQQKPGK |
| | | ISYSGYTTYNPSLKSRF | APKLLIYGATSLET |
| | | TISRDTSKNTLYLQMNS | GVPSRFSGSGSGTD |
| | | LRAEDTAVYYCARWTSG | FTLTISSLQPEDFA |
| | | LDYWGQGTLVTVSSAST | TYYCQQYWTTPFTF |
| | | KGPSVFPLAPSSKSTSG | GQGTKVEIKRTVAA |
| | | GTAALGCLVKDYFPCPV | PSVFIFPPSDEQLK |
| | | TVSWNSGALTSGVHTFP | SGTASVVCLLNNFY |
| | | AVLQSSGLYSLSSVVTV | PREAKVQWKVDNAL |
| | | PSSSLGTQTYICNVNHK | QSGNSQESVTEQDS |
| | | PSNTKVDKRVEPKSCDK | KDSTYSLSSTLTLS |
| | | THTCPPCPAPELLGGPS | KADYEKHKVYACEV |
| | | VFLFPPKPKDTLMISRT | THQGLSSPVTKSFN |
| | | PEVTCVVVDVSHEDPEV | RGEC (SEQ ID |
| | | KFNWYVDGVEVHNAKTK | NO: 123) |
| | | PREEQYNSTYRVVSVLT | |
| | | VLHQDWLNGKEYKCKVS | |
| | | NKALPAPIEKTISKAKG | |
| | | QPREPQVYTLPPSREEM | |
| | | TKNQVSLTCLVKGFYPC | |
| | | DIAVEWESNGQPENNYK | |
| | | TTPPVLDSDGSFFLYSK | |
| | | LTVDKSRWQQGNVFSCS | |
| | | VMHEALHNHYTQKSLSL | |
| | | SPGK (SEQ ID | |
| | | NO: 122) | |
| EGFR2 | Aba | EVQLQESGPGLVKPSQT | DIQMTQSPSSMSVS |
| | | LSLTCTVSGYSISRDFA | VGDRVTITCHSSQD |
| | | WNWIRQPPGKGLEWMGY | INSNIGWLQQKPGK |
| | | ISYNGNTRYQPSLKSRI | SFKGLIYHGTNLDD |
| | | TISRDTSKNQFFLKLNS | GVPSRFSGSGSGTD |
| | | VTAADTATYYCVTASRG | YTLTISSLQPEDFA |
| | | FPYWGQGTLVTVSSAST | TYYCVQYAQFPWTF |
| | | KGPSVFPLAPSSKSTSG | GGGTKLEIKRTVAA |
| | | GTAALGCLVKDYFPCPV | PSVFIFPPSDEQLK |
| | | TVSWNSGALTSGVHTFP | SGTASVVCLLNNFY |
| | | AVLQSSGLYSLSSVVTV | PREAKVQWKVDNAL |
| | | PSSSLGTQTYICNVNHK | QSGNSQESVTEQDS |
| | | PSNTKVDKRVEPKSCDK | KDSTYSLSSTLTLS |
| | | THTCPPCPAPELLGGPS | KADYEKHKVYACEV |
| | | VFLFPPKPKDTLMISRT | THQGLSSPVTKSFN |
| | | PEVTCVVVDVSHEDPEV | RGEC (SEQ ID |
| | | KFNWYVDGVEVHNAKTK | NO: 125) |
| | | PREEQYNSTYRVVSVLT | |
| | | VLHQDWLNGKEYKCKVS | |
| | | NKALPAPIEKTISKAKG | |
| | | QPREPQVYTLPPSREEM | |
| | | TKNQVSLTCLVKGFYPC | |
| | | DIAVEWESNGQPENNYK | |
| | | TTPPVLDSDGSFFLYSK | |
| | | LTVDKSRWQQGNVFSCS | |
| | | VMHEALHNHYTQKSLSL | |
| | | SPGK (SEQ ID | |
| | | NO: 124) | |
| CD7 | Ab D | EVQLVESGGGLVKPGGS | DIQMTQTTSSLSAS |
| | | LKLSCAASGLTFSSYAM | LGDRVTISCSASQG |
| | | SWVRQTPEKRLEWVASI | ISNYLNWYQQKPDG |
| | | SSGGFTYYPDSVKGRFT | TVKLLIYYTSSLHS |
| | | ISRDNARNILYLQMSSL | GVPSRFSGSGSGTD |
| | | RSEDTAMYYCARDEVRG | YSLTISNLEPEDIA |
| | | YLDVWGAGTTVTVSSAS | TYYCQQYSKLPYTF |
| | | TKGPSVFPLAPSSKSTS | GGGTKLEIKRTVAA |
| | | GGTAALGCLVKDYFPCP | PSVFIFPPSDEQLK |
| | | VTVSWNSGALTSGVHTF | SGTASVVCLLNNFY |
| | | PAVLQSSGLYSLSSVVT | PREAKVQWKVDNAL |
| | | VPSSSLGTQTYICNVNH | QSGNSQESVTEQDS |
| | | KPSNTKVDKRVEPKSCD | KDSTYSLSSTLTLS |
| | | KTHTCPPCPAPELLGGP | KADYEKHKVYACEV |
| | | SVFLFPPKPKDTLMISR | THQGLSSPVTKSFN |
| | | TPEVTCVVVDVSHEDPE | RGEC (SEQ ID |
| | | VKFNWYVDGVEVHNAKT | NO: 144) |
| | | KPREEQYNSTYRVVSVL | |
| | | TVLHQDWLNGKEYKCKV | |
| | | SNKALPAPIEKTISKAK | |
| | | GQPREPQVYTLPPSREE | |
| | | MTKNQVSLTCLVKGFYP | |
| | | CDIAVEWESNGQPENNY | |
| | | KTTPPVLDSDGSFFLYS | |
| | | KLTVDKSRWQQGNVFSC | |
| | | SVMHEALHNHYTQKSLS | |
| | | LSPGK (SEQ ID | |
| | | NO: 143) | |

TABLE D6
Exemplary target antigen amino acid sequences

| Antigen | SEQ ID NO | Amino acid sequence |
|---|---|---|
| BCMA | 72 | MLQMAGQCSQNEYFDSLLHACIPCQLRCSSNTPPLTCQRYCNASV |
| | | TNSVKGTNAILWTCLGLSLIISLAVFVLMFLLRKINSEPLKDEFK |
| | | NTGSGLLGMANIDLEKSRTGDEIILPRGLEYTVEECTCEDCIKSK |
| | | PKVDSDHCFPLPAMEEGATILVTTKTNDYCKSLPAALSATEIEKS |
| | | ISAR |
| CD33 | 73 | MPLLLLLPLLWAGALAMDPNFWLQVQESVTVQEGLCVLVPCTFFH |
| | | PIPYYDKNSPVHGYWFREGAIISRDSPVATNKLDQEVQEETQGRF |
| | | RLLGDPSRNNCSLSIVDARRRDNGSYFFRMERGSTKYSYKSPQLS |
| | | VHVTDLTHRPKILIPGTLEPGHSKNLTCSVSWACEQGTPPIFSWL |
| | | SAAPTSLGPRTTHSSVLIITPRPQDHGTNLTCQVKFAGAGVTTER |
| | | TIQLNVTYVPQNPTTGIFPGDGSGKQETRAGVVHGAIGGAGVTAL |
| | | LALCLCLIFFIVKTHRRKAARTAVGRNDTHPTTGSASPKHQKKSK |
| | | LHGPTETSSCSGAAPTVEMDEELHYASLNFHGMNPSKDTSTEYSE |
| | | VRTQ |
| PCAD | 74 | MGLPRGPLASLLLLQVCWLQCAASEPCRAVFREAEVTLEAGGAEQ |
| | | EPGQALGKVFMGCPGQEPALFSTDNDDFTVRNGETVQERRSLKER |
| | | NPLKIFPSKRILRRHKRDWVVAPISVPENGKGPFPQRLNQLKSNK |
| | | DRDTKIFYSITGPGADSPPEGVFAVEKETGWLLLNKPLDREEIAK |
| | | YELFGHAVSENGASVEDPMNISIIVTDQNDHKPKFTQDTFRGSVL |
| | | EGVLPGTSVMQVTATDEDDAIYTYNGVVAYSIHSQEPKDPHDLMF |
| | | TIHRSTGTISVISSGLDREKVPEYTLTIQATDMDGDGSTTTAVAV |
| | | VEILDANDNAPMFDPQKYEAHVPENAVGHEVQRLTVTDLDAPNSP |
| | | AWRATYLIMGGDDGDHFTITTHPESNQGILTTRKGLDFEAKNQHT |
| | | LYVEVTNEAPFVLKLPTSTATIVVHVEDVNEAPVFVPPSKVVEVQ |
| | | EGIPTGEPVCVYTAEDPDKENQKISYRILRDPAGWLAMDPDSGQV |
| | | TAVGTLDREDEQFVRNNIYEVMVLAMDNGSPPTTGTGTLLLTLID |
| | | VNDHGPVPEPRQITICNQSPVRQVLNITDKDLSPHTSPFQAQLTD |
| | | DSDIYWTAEVNEEGDTVVLSLKKFLKQDTYDVHLSLSDHGNKEQL |
| | | TVIRATVCDCHGHVETCPGPWKGGFILPVLGAVLALLFLLLVLLL |
| | | LVRKKRKIKEPLLLPEDDTRDNVFYYGEEGGGEEDQDYDITQLHR |
| | | GLEARPEVVLRNDVAPTIIPTPMYRPRPANPDEIGNFIIENLKAA |
| | | NTDPTAPPYDTLLVFDYEGSGSDAASLSSLTSSASDQDQDYDYLN |
| | | EWGSRFKKLADMYGGGEDD |
| HER2/NEU | 75 | MELAALCRWGLLLALLPPGAASTQVCTGTDMKLRLPASPETHLDM |
| | | LRHLYQGCQVVQGNLELTYLPTNASLSFLQDIQEVQGYVLIAHNQ |
| | | VRQVPLQRLRIVRGTQLFEDNYALAVLDNGDPLNNTTPVTGASPG |
| | | GLRELQLRSLTEILKGGVLIQRNPQLCYQDTILWKDIFHKNNQLA |
| | | LTLIDTNRSRACHPCSPMCKGSRCWGESSEDCQSLTRTVCAGGCA |
| | | RCKGPLPTDCCHEQCAAGCTGPKHSDCLACLHFNHSGICELHCPA |
| | | LVTYNTDTFESMPNPEGRYTFGASCVTACPYNYLSTDVGSCTLVC |
| | | PLHNQEVTAEDGTQRCEKCSKPCARVCYGLGMEHLREVRAVTSAN |
| | | IQEFAGCKKIFGSLAFLPESFDGDPASNTAPLQPEQLQVFETLEE |
| | | ITGYLYISAWPDSLPDLSVFQNLQVIRGRILHNGAYSLTLQGLGI |
| | | SWLGLRSLRELGSGLALIHHNTHLCFVHTVPWDQLFRNPHQALLH |
| | | TANRPEDECVGEGLACHQLCARGHCWGPGPTQCVNCSQFLRGQEC |
| | | VEECRVLQGLPREYVNARHCLPCHPECQPQNGSVTCFGPEADQCV |
| | | ACAHYKDPPFCVARCPSGVKPDLSYMPIWKFPDEEGACQPCPINC |
| | | THSCVDLDDKGCPAEQRASPLTSIISAVVGILLVVVLGVVFGILI |
| | | KRRQQKIRKYTMRRLLQETELVEPLTPSGAMPNQAQMRILKETEL |
| | | RKVKVLGSGAFGTVYKGIWIPDGENVKIPVAIKVLRENTSPKANK |
| | | EILDEAYVMAGVGSPYVSRLLGICLTSTVQLVTQLMPYGCLLDHV |
| | | RENRGRLGSQDLLNWCMQIAKGMSYLEDVRLVHRDLAARNVLVKS |
| | | PNHVKITDFGLARLLDIDETEYHADGGKVPIKWMALESILRRRFT |
| | | HQSDVWSYGVTVWELMTFGAKPYDGIPAREIPDLLEKGERLPQPP |
| | | ICTIDVYMIMVKCWMIDSECRPRFRELVSEFSRMARDPQRFVVIQ |
| | | NEDLGPASPLDSTFYRSLLEDDDMGDLVDAEEYLVPQQGFFCPDP |
| | | APGAGGMVHHRHRSSSTRSGGGDLTLGLEPSEEEAPRSPLAPSEG |
| | | AGSDVFDGDLGMGAAKGLQSLPTHDPSPLQRYSEDPTVPLPSETD |
| | | GYVAPLTCSPQPEYVNQPDVRPQPPSPREGPLPAARPAGATLERP |
| | | KTLSPGKNGVVKDVFAFGGAVENPEYLTPQGGAAPQPHPPPAFSP |
| | | AFDNLYYWDQDPPERGAPPSTFKGTPTAENPEYLGLDVPV |
| CD38 | 76 | MANCEFSPVSGDKPCCRLSRRAQLCLGVSILVLILVVVLAVVVPR |
| | | WRQQWSGPGTTKRFPETVLARCVKYTEIHPEMRHVDCQSVWDAFK |
| | | GAFISKHPCNITEEDYQPLMKLGTQTVPCNKILLWSRIKDLAHQF |
| | | TQVQRDMFTLEDTLLGYLADDLTWCGEFNTSKINYQSCPDWRKDC |
| | | SNNPVSVFWKTVSRRFAEAACDVVHVMLNGSRSKIFDKNSTFGSV |
| | | EVHNLQPEKVQTLEAWVIHGGREDSRDLCQDPTIKELESIISKRN |
| | | IQFSCKNIYRPDKFLQCVKNPEDSSCTSEI |
| CD48 | 77 | MCSRGWDSCLALELLLLPLSLLVTSIQGHLVHMTVVSGSNVTLNI |
| | | SESLPENYKQLTWFYTFDQKIVEWDSRKSKYFESKFKGRVRLDPQ |
| | | SGALYISKVQKEDNSTYIMRVLKKTGNEQEWKIKLQVLDPVPKPV |
| | | IKIEKIEDMDDNCYLKLSCVIPGESVNYTWYGDKRPFPKELQNSV |
| | | LETTLMPHNYSRCYTCQVSNSVSSKNGTVCLSPPCTLARSFGVEW |
| | | IASWLVVTVPTILGLLLT |
| CD79b | 78 | MARLALSPVPSHWMVALLLLLSAEPVPAARSEDRYRNPKGSACSR |
| | | IWQSPRFIARKRGFTVKMHCYMNSASGNVSWLWKQEMDENPQQLK |
| | | LEKGRMEESQNESLATLTIQGIRFEDNGIYFCQQKCNNTSEVYQG |
| | | CGTELRVMGFSTLAQLKQRNTLKDGIIMIQTLLIILFIIVPIFLL |
| | | LDKDDSKAGMEEDHTYEGLDIDQTATYEDIVTLRTGEVKWSVGEH |
| | | PGQE |
| EGFR | 126 | MRPSGTAGAALLALLAALCPASRALEEKKVCQGTSNKLTQLGTFE |
| (NP_005219.2) | | DHFLSLQRMFNNCEVVLGNLEITYVQRNYDLSFLKTIQEVAGYVL |
| | | IALNTVERIPLENLQIIRGNMYYENSYALAVLSNYDANKTGLKEL |
| | | PMRNLQEILHGAVRFSNNPALCNVESIQWRDIVSSDFLSNMSMDF |
| | | QNHLGSCQKCDPSCPNGSCWGAGEENCQKLTKIICAQQCSGRCRG |
| | | KSPSDCCHNQCAAGCTGPRESDCLVCRKFRDEATCKDTCPPLMLY |
| | | NPTTYQMDVNPEGKYSFGATCVKKCPRNYVVTDHGSCVRACGADS |
| | | YEMEEDGVRKCKKCEGPCRKVCNGIGIGEFKDSLSINATNIKHFK |
| | | NCTSISGDLHILPVAFRGDSFTHTPPLDPQELDILKTVKEITGFL |
| | | LIQAWPENRTDLHAFENLEIIRGRTKQHGQFSLAVVSLNITSLGL |
| | | RSLKEISDGDVIISGNKNLCYANTINWKKLFGTSGQKTKIISNRG |
| | | ENSCKATGQVCHALCSPEGCWGPEPRDCVSCRNVSRGRECVDKCN |
| | | LLEGEPREFVENSECIQCHPECLPQAMNITCTGRGPDNCIQCAHY |
| | | IDGPHCVKTCPAGVMGENNTLVWKYADAGHVCHLCHPNCTYGCTG |
| | | PGLEGCPTNGPKIPSIATGMVGALLLLLVVALGIGLFMRRRHIVR |
| | | KRTLRRLLQERELVEPLTPSGEAPNQALLRILKETEFKKIKVLGS |
| | | GAFGTVYKGLWIPEGEKVKIPVAIKELREATSPKANKEILDEAYV |
| | | MASVDNPHVCRLLGICLTSTVQLITQLMPFGCLLDYVREHKDNIG |
| | | SQYLLNWCVQIAKGMNYLEDRRLVHRDLAARNVLVKTPQHVKITD |
| | | FGLAKLLGAEEKEYHAEGGKVPIKWMALESILHRIYTHQSDVWSY |
| | | GVTVWELMTFGSKPYDGIPASEISSILEKGERLPQPPICTIDVYM |
| | | IMVKCWMIDADSRPKFRELIIEFSKMARDPQRYLVIQGDERMHLP |
| | | SPTDSNFYRALMDEEDMDDVVDADEYLIPQQGFFSSPSTSRTPLL |
| | | SSLSATSNNSTVACIDRNGLQSCPIKEDSFLQRYSSDPTGALTED |
| | | SIDDTFLPVPEYINQSVPKRPAGSVQNPVYHNQPLNPAPSRDPHY |
| | | QDPHSTAVGNPEYLNTVQPTCVNSTFDSPAHWAQKGSHQISLDNP |
| | | DYQQDFFPKEAKPNGIFKGSTAENAEYLRVAPQSSEFIGA |
| CD7 | 127 | MAGPPRLLLLPLLLALARGLPGALAAQEVQQSPHCTTVPVGASVN |
| (NP_006128.1) | | ITCSTSGGLRGIYLRQLGPQPQDIIYYEDGVVPTTDRRFRGRIDF |
| | | SGSQDNLTITMHRLQLSDTGTYTCQAITEVNVYGSGTLVLVTEEQ |
| | | SQGWHRCSDAPPRASALPAPPTGSALPDPQTASALPDPPAASALP |
| | | AALAVISFLLGLGLGVACVLARTQIKKLCSWRDKNSAACVVYEDM |
| | | SHSRCNTLSSPNQYQ |
| TFRC | 128 | MMDQARSAFSNLFGGEPLSYTRFSLARQVDGDNSHVEMKLAVDEE |
| (NP_003225.2) | | ENADNNTKANVTKPKRCSGSICYGTIAVIVFFLIGFMIGYLGYCK |
| | | GVEPKTECERLAGTESPVREEPGEDFPAARRLYWDDLKRKLSEKL |
| | | DSTDFTGTIKLLNENSYVPREAGSQKDENLALYVENQFREFKLSK |
| | | VWRDQHFVKIQVKDSAQNSVIIVDKNGRLVYLVENPGGYVAYSKA |
| | | ATVTGKLVHANFGTKKDFEDLYTPVNGSIVIVRAGKITFAEKVAN |
| | | AESLNAIGVLIYMDQTKFPIVNAELSFFGHAHLGTGDPYTPGFPS |
| | | FNHTQFPPSRSSGLPNIPVQTISRAAAEKLFGNMEGDCPSDWKTD |
| | | STCRMVTSESKNVKLTVSNVLKEIKILNIFGVIKGFVEPDHYVVV |
| | | GAQRDAWGPGAAKSGVGTALLLKLAQMFSDMVLKDGFQPSRSIIF |
| | | ASWSAGDFGSVGATEWLEGYLSSLHLKAFTYINLDKAVLGTSNFK |
| | | VSASPLLYTLIEKTMQNVKHPVTGQFLYQDSNWASKVEKLTLDNA |
| | | AFPFLAYSGIPAVSFCFCEDTDYPYLGTTMDTYKELIERIPELNK |
| | | VARAAAEVAGQFVIKLTHDVELNLDYERYNSQLLSFVRDLNQYRA |
| | | DIKEMGLSLQWLYSARGDFFRATSRLTTDFGNAEKTDRFVMKKLN |
| | | DRVMRVEYHFLSPYVSPKESPFRHVFWGSGSHTLPALLENLKLRK |
| | | QNNGAFNETLFRNQLALATWTIQGAANALSGDVWDIDNEF |
| EPCAM | 129 | MAPPQVLAFGLLLAAATATFAAAQEECVCENYKLAVNCFVNNNRQ |
| (NP_002345.2) | | CQCTSVGAQNTVICSKLAAKCLVMKAEMNGSKLGRRAKPEGALQN |
| | | NDGLYDPDCDESGLFKAKQCNGTSMCWCVNTAGVRRTDKDTEITC |
| | | SERVRTYWIIIELKHKAREKPYDSKSLRTALQKEITTRYQLDPKF |
| | | ITSILYENNVITIDLVQNSSQKTQNDVDIADVAYYFEKDVKGESL |
| | | FHSKKMDLTVNGEQLDLDPGQTLIYYVDEKAPEFSMQGLKAGVIA |
| | | VIVVVVIAVVAGIVVLVISRKKRMAKYEKAEIKEMGEMHRELNA |
| FOLR1 | 130 | MAQRMTTQLLLLLVWVAVVGEAQTRIAWARTELLNVCMNAKHHKE |
| (NP_057936.1) | | KPGPEDKLHEQCRPWRKNACCSTNTSQEAHKDVSYLYRFNWNHCG |
| | | EMAPACKRHFIQDTCLYECSPNLGPWIQQVDQSWRKERVLNVPLC |
| | | KEDCEQWWEDCRTSYTCKSNWHKGWNWTSGFNKCAVGAACQPFHF |
| | | YFPTPTVLCNEIWTHSYKVSNYSRGSGRCIQMWFDPAQGNPNEEV |
| | | ARFYAAAMSGAGPWAAWPFLLSLALMLLWLLS |
| ENPP3 | 131 | MESTLTLATEQPVKKNTLKKYKIACIVLLALLVIMSLGLGLGLGL |
| (NP_005012.2) | | RKLEKQGSCRKKCFDASFRGLENCRCDVACKDRGDCCWDFEDTCV |
| | | ESTRIWMCNKFRCGETRLEASLCSCSDDCLQRKDCCADYKSVCQG |
| | | ETSWLEENCDTAQQSQCPEGFDLPPVILFSMDGFRAEYLYTWDTL |
| | | MPNINKLKTCGIHSKYMRAMYPTKTFPNHYTIVTGLYPESHGIID |
| | | NNMYDVNLNKNFSLSSKEQNNPAWWHGQPMWLTAMYQGLKAATYF |
| | | WPGSEVAINGSFPSIYMPYNGSVPFEERISTLLKWLDLPKAERPR |
| | | FYTMYFEEPDSSGHAGGPVSARVIKALQVVDHAFGMLMEGLKQRN |
| | | LHNCVNIILLADHGMDQTYCNKMEYMTDYFPRINFFYMYEGPAPR |
| | | IRAHNIPHDFFSFNSEEIVRNLSCRKPDQHFKPYLTPDLPKRLHY |
| | | AKNVRIDKVHLFVDQQWLAVRSKSNTNCGGGNHGYNNEFRSMEAI |
| | | FLAHGPSFKEKTEVEPFENIEVYNLMCDLLRIQPAPNNGTHGSLN |
| | | HLLKVPFYEPSHAEEVSKFSVCGFANPLPTESLDCFCPHLQNSTQ |
| | | LEQVNQMLNLTQEEITATVKVNLPFGRPRVLQKNVDHCLLYHREY |
| | | VSGFGKAMRMPMWSSYTVPQLGDTSPLPPTVPDCLRADVRVPPSE |
| | | SQKCSFYLADKNITHGFLYPPASNRTSDSQYDALITSNLVPMYEE |
| | | FRKMWDYFHSVLLIKHATERNGVNVVSGPIFDYNYDGHFDAPDEI |
| | | TKHLANTDVPIPTHYFVVLTSCKNKSHTPENCPGWLDVLPFIIPH |
| | | RPTNVESCPEGKPEALWVEERFTAHIARVRDVELLTGLDFYQDKV |
| | | QPVSEILQLKTYLPTFETTI |
| MET | 132 | MKAPAVLAPGILVLLFTLVQRSNGECKEALAKSEMNVNMKYQLPN |
| (NP_00112097 | | FTAETPIQNVILHEHHIFLGATNYIYVLNEEDLQKVAEYKTGPVL |
| 2.1) | | EHPDCFPCQDCSSKANLSGGVWKDNINMALVVDTYYDDQLISCGS |
| | | VNRGTCQRHVFPHNHTADIQSEVHCIFSPQIEEPSQCPDCVVSAL |
| | | GAKVLSSVKDRFINFFVGNTINSSYFPDHPLHSISVRRLKETKDG |
| | | FMFLTDQSYIDVLPEFRDSYPIKYVHAFESNNFIYFLTVQRETLD |
| | | AQTFHTRIIRFCSINSGLHSYMEMPLECILTEKRKKRSTKKEVFN |
| | | ILQAAYVSKPGAQLARQIGASLNDDILFGVFAQSKPDSAEPMDRS |
| | | AMCAFPIKYVNDFFNKIVNKNNVRCLQHFYGPNHEHCFNRTLLRN |
| | | SSGCEARRDEYRTEFTTALQRVDLFMGQFSEVLLTSISTFIKGDL |
| | | TIANLGTSEGRFMQVVVSRSGPSTPHVNFLLDSHPVSPEVIVEHT |
| | | LNQNGYTLVITGKKITKIPLNGLGCRHFQSCSQCLSAPPFVQCGW |
| | | CHDKCVRSEECLSGTWTQQICLPAIYKVFPNSAPLEGGTRLTICG |
| | | WDFGFRRNNKFDLKKTRVLLGNESCTLTLSESTMNTLKCTVGPAM |
| | | NKHFNMSIIISNGHGTTQYSTFSYVDPVITSISPKYGPMAGGTLL |
| | | TLTGNYLNSGNSRHISIGGKTCTLKSVSNSILECYTPAQTISTEF |
| | | AVKLKIDLANRETSIFSYREDPIVYEIHPTKSFISTWWKEPLNIV |
| | | SFLFCFASGGSTITGVGKNLNSVSVPRMVINVHEAGRNFTVACQH |
| | | RSNSEIICCTTPSLQQLNLQLPLKTKAFFMLDGILSKYFDLIYVH |
| | | NPVFKPFEKPVMISMGNENVLEIKGNDIDPEAVKGEVLKVGNKSC |
| | | ENIHLHSEAVLCTVPNDLLKLNSELNIEWKQAISSTVLGKVIVQP |
| | | DQNFTGLIAGVVSISTALLLLLGFFLWLKKRKQIKDLGSELVRYD |
| | | ARVHTPHLDRLVSARSVSPTTEMVSNESVDYRATFPEDQFPNSSQ |
| | | NGSCRQVQYPLTDMSPILTSGDSDISSPLLQNTVHIDLSALNPEL |
| | | VQAVQHVVIGPSSLIVHFNEVIGRGHFGCVYHGTLLDNDGKKIHC |
| | | AVKSLNRITDIGEVSQFLTEGIIMKDFSHPNVLSLLGICLRSEGS |
| | | PLVVLPYMKHGDLRNFIRNETHNPTVKDLIGFGLQVAKGMKYLAS |
| | | KKFVHRDLAARNCMLDEKFTVKVADFGLARDMYDKEYYSVHNKTG |
| | | AKLPVKWMALESLQTQKFTTKSDVWSFGVLLWELMTRGAPPYPDV |
| | | NTFDITVYLLQGRRLLQPEYCPDPLYEVMLKCWHPKAEMRPSFSE |
| | | LVSRISAIFSTFIGEHYVHVNATYVNVKCVAPYPSLLSSEDNADD |
| | | EVDTRPASFWETS |
| AXL | 133 | MAWRCPRMGRVPLAWCLALCGWACMAPRGTQAEESPFVGNPGNIT |
| (NP_068713.2) | | GARGLTGTLRCQLQVQGEPPEVHWLRDGQILELADSTQTQVPLGE |
| | | DEQDDWIVVSQLRITSLQLSDTGQYQCLVFLGHQTFVSQPGYVGL |
| | | EGLPYFLEEPEDRTVAANTPFNLSCQAQGPPEPVDLLWLQDAVPL |
| | | ATAPGHGPQRSLHVPGLNKTSSFSCEAHNAKGVTTSRTATITVLP |
| | | QQPRNLHLVSRQPTELEVAWTPGLSGIYPLTHCTLQAVLSDDGMG |
| | | IQAGEPDPPEEPLTSQASVPPHQLRLGSLHPHTPYHIRVACTSSQ |
| | | GPSSWTHWLPVETPEGVPLGPPENISATRNGSQAFVHWQEPRAPL |
| | | QGTLLGYRLAYQGQDTPEVLMDIGLRQEVTLELQGDGSVSNLTVC |
| | | VAAYTAAGDGPWSLPVPLEAWRPGQAQPVHQLVKEPSTPAFSWPW |
| | | WYVLLGAVVAAACVLILALFLVHRRKKETRYGEVFEPTVERGELV |
| | | VRYRVRKSYSRRTTEATLNSLGISEELKEKLRDVMVDRHKVALGK |
| | | TLGEGEFGAVMEGQLNQDDSILKVAVKTMKIAICTRSELEDFLSE |
| | | AVCMKEFDHPNVMRLIGVCFQGSERESFPAPVVILPFMKHGDLHS |
| | | FLLYSRLGDQPVYLPTQMLVKFMADIASGMEYLSTKRFIHRDLAA |
| | | RNCMLNENMSVCVADFGLSKKIYNGDYYRQGRIAKMPVKWIAIES |
| | | LADRVYTSKSDVWSFGVTMWEIATRGQTPYPGVENSEIYDYLRQG |
| | | NRLKQPADCLDGLYALMSRCWELNPQDRPSFTELREDLENTLKAL |
| | | PPAQEPDEILYVNMDEGGGYPEPPGAAGGADPPTQPDPKDSCSCL |
| | | TAAEVHPAGRYVLCPSTTPSPAQPADRGSPAAPGQEDGA |
| SLC34A2 | 134 | MAPWPELGDAQPNPDKYLEGAAGQQPTAPDKSKETNKTDNTEAPV |
| (NP_006415.3) | | TKIELLPSYSTATLIDEPTEVDDPWNLPTLQDSGIKWSERDTKGK |
| | | ILCFFQGIGRLILLLGFLYFFVCSLDILSSAFQLVGGKMAGQFFS |
| | | NSSIMSNPLLGLVIGVLVTVLVQSSSTSTSIVVSMVSSSLLTVRA |
| | | AIPIIMGANIGTSITNTIVALMQVGDRSEFRRAFAGATVHDFFNW |
| | | LSVLVLLPVEVATHYLEIITQLIVESFHFKNGEDAPDLLKVITKP |
| | | FTKLIVQLDKKVISQIAMNDEKAKNKSLVKIWCKTFTNKTQINVT |
| | | VPSTANCTSPSLCWTDGIQNWTMKNVTYKENIAKCQHIFVNFHLP |
| | | DLAVGTILLILSLLVLCGCLIMIVKILGSVLKGQVATVIKKTINT |
| | | DFPFPFAWLTGYLAILVGAGMTFIVQSSSVFTSALTPLIGIGVIT |
| | | IERAYPLTLGSNIGTTTTAILAALASPGNALRSSLQIALCHFFFN |
| | | ISGILLWYPIPFTRLPIRMAKGLGNISAKYRWFAVFYLIIFFFLI |
| | | PLTVFGLSLAGWRVLVGVGVPVVFIIILVLCLRLLQSRCPRVLPK |
| | | KLQNWNFLPLWMRSLKPWDAVVSKFTGCFQMRCCCCCRVCCRACC |
| | | LLCDCPKCCRCSKCCEDLEEAQEGQDVPVKAPETFDNITISREAQ |
| | | GEVPASDSKTECTAL |
| NECTIN4 | 135 | MPLSLGAEMWGPEAWLLLLLLLASFTGRCPAGELETSDVVTVVLG |
| (NP_112178.2) | | QDAKLPCFYRGDSGEQVGQVAWARVDAGEGAQELALLHSKYGLHV |
| | | SPAYEGRVEQPPPPRNPLDGSVLLRNAVQADEGEYECRVSTFPAG |
| | | SFQARLRLRVLVPPLPSLNPGPALEEGQGLTLAASCTAEGSPAPS |
| | | VTWDTEVKGTTSSRSFKHSRSAAVTSEFHLVPSRSMNGQPLTCVV |
| | | SHPGLLQDQRITHILHVSFLAEASVRGLEDQNLWHIGREGAMLKC |
| | | LSEGQPPPSYNWTRLDGPLPSGVRVDGDTLGFPPLTTEHSGIYVC |
| | | HVSNEFSSRDSQVTVDVLDPQEDSGKQVDLVSASVVVVGVIAALL |
| | | FCLLVVVVVLMSRYHRRKAQQMTQKYEEELTLTRENSIRRLHSHH |
| | | TDPRSQPEESVGLRAEGHPDSLKDNSSCSVMSEEPEGRSYSTLTT |
| | | VREIETQTELLSPGSGRAEEEEDQDEGIKQAMNHFVQENGTLRAK |
| | | PTGNGIYINGRGHLV |
| TACSTD2 | 136 | MARGPGLAPPPLRLPLLLLVLAAVTGHTAAQDNCTCPTNKMTVCS |
| (NP_002344.2) | | PDGPGGRCQCRALGSGMAVDCSTLTSKCLLLKARMSAPKNARTLV |
| | | RPSEHALVDNDGLYDPDCDPEGRFKARQCNQTSVCWCVNSVGVRR |
| | | TDKGDLSLRCDELVRTHHILIDLRHRPTAGAFNHSDLDAELRRLF |
| | | RERYRLHPKFVAAVHYEQPTIQIELRQNTSQKAAGDVDIGDAAYY |
| | | FERDIKGESLFQGRGGLDLRVRGEPLQVERTLIYYLDEIPPKFSM |
| | | KRLTAGLIAVIVVVVVALVAGMAVLVITNRRKSGKYKKVEIKELG |
| | | ELRKEPSL |
| SLC39A6 | 137 | MARKLSVILILTFALSVTNPLHELKAAAFPQTTEKISPNWESGIN |
| (NP_036451.4) | | VDLAISTRQYHLQQLFYRYGENNSLSVEGFRKLLQNIGIDKIKRI |
| | | HIHHDHDHHSDHEHHSDHERHSDHEHHSEHEHHSDHDHHSHHNHA |
| | | ASGKNKRKALCPDHDSDSSGKDPRNSQGKGAHRPEHASGRRNVKD |
| | | SVSASEVTSTVYNTVSEGTHFLETIETPRPGKLFPKDVSSSTPPS |
| | | VTSKSRVSRLAGRKTNESVSEPRKGFMYSRNTNENPQECFNASKL |
| | | LTSHGMGIQVPLNATEFNYLCPAIINQIDARSCLIHTSEKKAEIP |
| | | PKTYSLQIAWVGGFIAISIISFLSLLGVILVPLMNRVFFKFLLSF |
| | | LVALAVGTLSGDAFLHLLPHSHASHHHSHSHEEPAMEMKRGPLFS |
| | | HLSSQNIEESAYFDSTWKGLTALGGLYFMFLVEHVLTLIKQFKDK |
| | | KKKNQKKPENDDDVEIKKQLSKYESQLSTNEEKVDTDDRTEGYLR |
| | | ADSQEPSHFDSQQPAVLEEEEVMIAHAHPQEVYNEYVPRGCKNKC |
| | | HSHFHDTLGQSDDLIHHHHDYHHILHHHHHQNHHPHSHSQRYSRE |
| | | ELKDAGVATLAWMVIMGDGLHNFSDGLAIGAAFTEGLSSGLSTSV |
| | | AVFCHELPHELGDFAVLLKAGMTVKQAVLYNALSAMLAYLGMATG |
| | | IFIGHYAENVSMWIFALTAGLFMYVALVDMVPEMLHNDASDHGCS |
| | | RWGYFFLQNAGMLLGFGIMLLISIFEHKIVFRINF |
| GPNMB | 138 | MECLYYFLGFLLLAARLPLDAAKRFHDVLGNERPSAYMREHNQLN |
| (NP_00100534 | | GWSSDENDWNEKLYPVWKRGDMRWKNSWKGGRVQAVLTSDSPALV |
| 0.1) | | GSNITFAVNLIFPRCQKEDANGNIVYEKNCRNEAGLSADPYVYNW |
| | | TAWSEDSDGENGTGQSHHNVFPDGKPFPHHPGWRRWNFIYVFHTL |
| | | GQYFQKLGRCSVRVSVNTANVTLGPQLMEVTVYRRHGRAYVPIAQ |
| | | VKDVYVVTDQIPVFVTMFQKNDRNSSDETFLKDLPIMFDVLIHDP |
| | | SHFLNYSTINYKWSFGDNTGLFVSTNHTVNHTYVLNGTFSLNLTV |
| | | KAAAPGPCPPPPPPPRPSKPTPSLATTLKSYDSNTPGPAGDNPLE |
| | | LSRIPDENCQINRYGHFQATITIVEGILEVNIIQMTDVLMPVPWP |
| | | ESSLIDFVVTCQGSIPTEVCTIISDPTCEITQNTVCSPVDVDEMC |
| | | LLTVRRTFNGSGTYCVNLTLGDDTSLALTSTLISVPDRDPASPLR |
| | | MANSALISVGCLAIFVTVISLLVYKKHKEYNPIENSPGNVVRSKG |
| | | LSVFLNRAKAVFFPGNQEKDPLLKNQEFKGVS |
| MSLN | 139 | MALPTARPLLGSCGTPALGSLLFLLFSLGWVQPSRTLAGETGQEA |
| (NP_005814.2) | | APLDGVLANPPNISSLSPRQLLGFPCAEVSGLSTERVRELAVALA |
| | | QKNVKLSTEQLRCLAHRLSEPPEDLDALPLDLLLFLNPDAFSGPQ |
| | | ACTRFFSRITKANVDLLPRGAPERQRLLPAALACWGVRGSLLSEA |
| | | DVRALGGLACDLPGRFVAESAEVLLPRLVSCPGPLDQDQQEAARA |
| | | ALQGGGPPYGPPSTWSVSTMDALRGLLPVLGQPIIRSIPQGIVAA |
| | | WRQRSSRDPSWRQPERTILRPRFRREVEKTACPSGKKAREIDESL |
| | | IFYKKWELEACVDAALLATQMDRVNAIPFTYEQLDVLKHKLDELY |
| | | PQGYPESVIQHLGYLFLKMSPEDIRKWNVTSLETLKALLEVNKGH |
| | | EMSPQVATLIDRFVKGRGQLDKDTLDTLTAFYPGYLCSLSPEELS |
| | | SVPPSSIWAVRPQDLDTCDPRQLDVLYPKARLAFQNMNGSEYFVK |
| | | IQSFLGGAPTEDLKALSQQNVSMDLATFMKLRTDAVLPLTVAEVQ |
| | | KLLGPHVEGLKAEERHRPVRDWILRQRQDDLDTLGLGLQGGIPNG |
| | | YLVLDLSMQEALSGTPCLLGPGPVLTVLALLLASTLA |
| CD74 | 140 | MHRRRSRSCREDQKPVMDDQRDLISNNEQLPMLGRRPGAPESKCS |
| (NP_00102033 | | RGALYTGFSILVTLLLAGQATTAYFLYQQQGRLDKLTVTSQNLQL |
| 0.1) | | ENLRMKLPKPPKPVSKMRMATPLLMQALPMGALPQGPMQNATKYG |
| | | NMTEDHVMHLLQNADPLKVYPPLKGSFPENLRHLKNTMETIDWKV |
| | | FESWMHHWLLFEMSRHSLEQKPTDAPPKVLTKCQEEVSHIPAVHP |
| | | GSFRPKCDENGNYLPLQCYGSIGYCWCVFPNGTEVPNTRSRGHHN |
| | | CSESLELEDPSSGLGVTKQDLGPVPM |
| F3 | 141 | METPAWPRVPRPETAVARTLLLGWVFAQVAGASGTTNTVAAYNLT |
| (NP_001984.1) | | WKSTNFKTILEWEPKPVNQVYTVQISTKSGDWKSKCFYTTDTECD |
| | | LTDEIVKDVKQTYLARVFSYPAGNVESTGSAGEPLYENSPEFTPY |
| | | LETNLGQPTIQSFEQVGTKVNVTVEDERTLVRRNNTFLSLRDVFG |
| | | KDLIYTLYYWKSSSSGKKTAKTNTNEFLIDVDKGENYCFSVQAVI |
| | | PSRTVNRKSTDSPVECMGQEKGEFREIFYIIGAVVFVVIILVIIL |
| | | AISLHKCRKAGVGQSWKENSPLNVS |
| MUC16 | 142 | MLKPSGLPGSSSPTRSLMTGSRSTKATPEMDSGLTGATLSPKTST |
| (NP_078966.2) | | GAIVVTEHTLPFTSPDKTLASPTSSVVGRTTQSLGVMSSALPEST |
| | | SRGMTHSEQRTSPSLSPQVNGTPSRNYPATSMVSGLSSPRTRTSS |
| | | TEGNFTKEASTYTLTVETTSGPVTEKYTVPTETSTTEGDSTETPW |
| | | DTRYIPVKITSPMKTFADSTASKENAPVSMTPAETTVTDSHTPGR |
| | | TNPSFGTLYSSFLDLSPKGTPNSRGETSLELILSTTGYPFSSPEP |
| | | GSAGHSRISTSAPLSSSASVLDNKISETSIFSGQSLTSPLSPGVP |
| | | EARASTMPNSAIPFSMTLSNAETSAERVRSTISSLGTPSISTKQT |
| | | AETILTFHAFAETMDIPSTHIAKTLASEWLGSPGTLGGTSTSALT |
| | | TTSPSTTLVSEETNTHHSTSGKETEGTLNTSMTPLETSAPGEESE |
| | | MTATLVPTLGFTTLDSKIRSPSQVSSSHPTRELRTTGSTSGRQSS |
| | | STAAHGSSDILRATTSSTSKASSWTSESTAQQFSEPQHTQWVETS |
| | | PSMKTERPPASTSVAAPITTSVPSVVSGFTTLKTSSTKGIWLEET |
| | | SADTLIGESTAGPTTHQFAVPTGISMTGGSSTRGSQGTTHLLTRA |
| | | TASSETSADLTLATNGVPVSVSPAVSKTAAGSSPPGGTKPSYTMV |
| | | SSVIPETSSLQSSAFREGTSLGLTPLNTRHPFSSPEPDSAGHTKI |
| | | STSIPLLSSASVLEDKVSATSTFSHHKATSSITTGTPEISTKTKP |
| | | SSAVLSSMTLSNAATSPERVRNATSPLTHPSPSGEETAGSVLTLS |
| | | TSAETTDSPNIHPTGTLTSESSESPSTLSLPSVSGVKTTFSSSTP |
| | | STHLFTSGEETEETSNPSVSQPETSVSRVRTTLASTSVPTPVFPT |
| | | MDTWPTRSAQFSSSHLVSELRATSSTSVINSTGSALPKISHLTGT |
| | | ATMSQTNRDTFNDSAAPQSTTWPETSPRFKTGLPSATTTVSTSAT |
| | | SLSATVMVSKFTSPATSSMEATSIREPSTTILTTETTNGPGSMAV |
| | | ASTNIPIGKGYITEGRLDTSHLPIGTTASSETSMDFTMAKESVSM |
| | | SVSPSQSMDAAGSSTPGRTSQFVDTFSDDVYHLTSREITIPRDGT |
| | | SSALTPQMTATHPPSPDPGSARSTWLGILSSSPSSPTPKVTMSST |
| | | FSTQRVTTSMIMDTVETSRWNMPNLPSTTSLTPSNIPTSGAIGKS |
| | | TLVPLDTPSPATSLEASEGGLPTLSTYPESTNTPSIHLGAHASSE |
| | | SPSTIKLTMASVVKPGSYTPLTFPSIETHIHVSTARMAYSSGSSP |
| | | EMTAPGETNTGSTWDPTTYITTTDPKDTSSAQVSTPHSVRTLRTT |
| | | ENHPKTESATPAAYSGSPKISSSPNLTSPATKAWTITDTTEHSTQ |
| | | LHYTKLAEKSSGFETQSAPGPVSVVIPTSPTIGSSTLELTSDVPG |
| | | EPLVLAPSEQTTITLPMATWLSTSLTEEMASTDLDISSPSSPMST |
| | | FAIFPPMSTPSHELSKSEADTSAIRNTDSTTLDQHLGIRSLGRTG |
| | | DLTTVPITPLTTTWTSVIEHSTQAQDTLSATMSPTHVTQSLKDQT |
| | | SIPASASPSHLTEVYPELGTQGRSSSEATTFWKPSTDTLSREIET |
| | | GPTNIQSTPPMDNTTTGSSSSGVTLGIAHLPIGTSSPAETSTNMA |
| | | LERRSSTATVSMAGTMGLLVTSAPGRSISQSLGRVSSVLSESTTE |
| | | GVTDSSKGSSPRINTQGNTALSSSLEPSYAEGSQMSTSIPLTSSP |
| | | TTPDVEFIGGSTFWTKEVTTVMTSDISKSSARTESSSATLMSTAL |
| | | GSTENTGKEKLRTASMDLPSPTPSMEVTPWISLTLSNAPNTTDSL |
| | | DLSHGVHTSSAGTLATDRSLNTGVTRASRLENGSDTSSKSLSMGN |
| | | STHTSMTYTEKSEVSSSIHPRPETSAPGAETTLTSTPGNRAISLT |
| | | LPFSSIPVEEVISTGITSGPDINSAPMTHSPITPPTIVWTSTGTI |
| | | EQSTQPLHAVSSEKVSVQTQSTPYVNSVAVSASPTHENSVSSGSS |
| | | TSSPYSSASLESLDSTISRRNAITSWLWDLTTSLPTTTWPSTSLS |
| | | EALSSGHSGVSNPSSTTTEFPLFSAASTSAAKQRNPETETHGPQN |
| | | TAASTLNTDASSVTGLSETPVGASISSEVPLPMAITSRSDVSGLT |
| | | SESTANPSLGTASSAGTKLTRTISLPTSESLVSFRMNKDPWTVSI |
| | | PLGSHPTTNTETSIPVNSAGPPGLSTVASDVIDTPSDGAESIPTV |
| | | SFSPSPDTEVTTISHFPEKTTHSFRTISSLTHELTSRVTPIPGDW |
| | | MSSAMSTKPTGASPSITLGERRTITSAAPTTSPIVLTASFTETST |
| | | VSLDNETTVKTSDILDARKTNELPSDSSSSSDLINTSIASSTMDV |
| | | TKTASISPTSISGMTASSSPSLFSSDRPQVPTSTTETNTATSPSV |
| | | SSNTYSLDGGSNVGGTPSTLPPFTITHPVETSSALLAWSRPVRTF |
| | | STMVSTDTASGENPTSSNSVVTSVPAPGTWTSVGSTTDLPAMGFL |
| | | KTSPAGEAHSLLASTIEPATAFTPHLSAAVVTGSSATSEASLLTT |
| | | SESKAIHSSPQTPTTPTSGANWETSATPESLLVVTETSDTTLTSK |
| | | ILVTDTILFSTVSTPPSKFPSTGTLSGASFPTLLPDTPAIPLTAT |
| | | EPTSSLATSFDSTPLVTIASDSLGTVPETTLTMSETSNGDALVLK |
| | | TVSNPDRSIPGITIQGVTESPLHPSSTSPSKIVAPRNTTYEGSIT |
| | | VALSTLPAGTTGSLVFSQSSENSETTALVDSSAGLERASVMPLTT |
| | | GSQGMASSGGIRSGSTHSTGTKTFSSLPLTMNPGEVTAMSEITTN |
| | | RLTATQSTAPKGIPVKPTSAESGLLTPVSASSSPSKAFASLTTAP |
| | | PTWGIPQSTLTFEFSEVPSLDTKSASLPTPGQSLNTIPDSDASTA |
| | | SSSLSKSPEKNPRARMMTSTKAISASSFQSTGFTETPEGSASPSM |
| | | AGHEPRVPTSGTGDPRYASESMSYPDPSKASSAMTSTSLASKLTT |
| | | LFSTGQAARSGSSSSPISLSTEKETSFLSPTASTSRKTSLFLGPS |
| | | MARQPNILVHLQTSALTLSPTSTLNMSQEEPPELTSSQTIAEEEG |
| | | TTAETQTLTFTPSETPTSLLPVSSPTEPTARRKSSPETWASSISV |
| | | PAKTSLVETTDGTLVTTIKMSSQAAQGNSTWPAPAEETGSSPAGT |
| | | SPGSPEMSTTLKIMSSKEPSISPEIRSTVRNSPWKTPETTVPMET |
| | | TVEPVTLQSTALGSGSTSISHLPTGTTSPTKSPTENMLATERVSL |
| | | SPSPPEAWTNLYSGTPGGTRQSLATMSSVSLESPTARSITGTGQQ |
| | | SSPELVSKTTGMEFSMWHGSTGGTTGDTHVSLSTSSNILEDPVTS |
| | | PNSVSSLTDKSKHKTETWVSTTAIPSTVLNNKIMAAEQQTSRSVD |
| | | EAYSSTSSWSDQTSGSDITLGASPDVTNTLYITSTAQTTSLVSLP |
| | | SGDQGITSLTNPSGGKTSSASSVTSPSIGLETLRANVSAVKSDIA |
| | | PTAGHLSQTSSPAEVSILDVTTAPTPGISTTITTMGTNSISTTTP |
| | | NPEVGMSTMDSTPATERRTTSTEHPSTWSSTAASDSWTVTDMTSN |
| | | LKVARSPGTISTMHTTSFLASSTELDSMSTPHGRITVIGTSLVTP |
| | | SSDASAVKTETSTSERTLSPSDTTASTPISTFSRVQRMSISVPDI |
| | | LSTSWTPSSTEAEDVPVSMVSTDHASTKTDPNTPLSTFLFDSLST |
| | | LDWDTGRSLSSATATTSAPQGATTPQELTLETMISPATSQLPFSI |
| | | GHITSAVTPAAMARSSGVTFSRPDPTSKKAEQTSTQLPTTTSAHP |
| | | GQVPRSAATTLDVIPHTAKTPDATFQRQGQTALTTEARATSDSWN |
| | | EKEKSTPSAPWITEMMNSVSEDTIKEVTSSSSVLRTLNTLDINLE |
| | | SGTTSSPSWKSSPYERIAPSESTTDKEAIHPSTNTVETTGWVTSS |
| | | EHASHSTIPAHSASSKLTSPVVTTSTREQAIVSMSTTTWPESTRA |
| | | RTEPNSFLTIELRDVSPYMDTSSTTQTSIISSPGSTAITKGPRTE |
| | | ITSSKRISSSFLAQSMRSSDSPSEAITRLSNFPAMTESGGMILAM |
| | | QTSPPGATSLSAPTLDTSATASWTGTPLATTQRFTYSEKTTLFSK |
| | | GPEDTSQPSPPSVEETSSSSSLVPIHATTSPSNILLTSQGHSPSS |
| | | TPPVTSVFLSETSGLGKTTDMSRISLEPGTSLPPNLSSTAGEALS |
| | | TYEASRDTKAIHHSADTAVTNMEATSSEYSPIPGHTKPSKATSPL |
| | | VTSHIMGDITSSTSVFGSSETTEIETVSSVNQGLQERSTSQVASS |
| | | ATETSTVITHVSSGDATTHVTKTQATFSSGTSISSPHQFITSTNT |
| | | FTDVSTNPSTSLIMTESSGVTITTQTGPTGAATQGPYLLDTSTMP |
| | | YLTETPLAVTPDFMQSEKTTLISKGPKDVSWTSPPSVAETSYPSS |
| | | LTPFLVTTIPPATSTLQGQHTSSPVSATSVLTSGLVKTTDMLNTS |
| | | MEPVTNSPQNLNNPSNEILATLAATTDIETIHPSINKAVTNMGTA |
| | | SSAHVLHSTLPVSSEPSTATSPMVPASSMGDALASISIPGSETTD |
| | | IEGEPTSSLTAGRKENSTLQEMNSTTESNIILSNVSVGAITEATK |
| | | MEVPSFDATFIPTPAQSTKFPDIFSVASSRLSNSPPMTISTHMTT |
| | | TQTGSSGATSKIPLALDTSTLETSAGTPSVVTEGFAHSKITTAMN |
| | | NDVKDVSQTNPPFQDEASSPSSQAPVLVTTLPSSVAFTPQWHSTS |
| | | SPVSMSSVLTSSLVKTAGKVDTSLETVTSSPQSMSNTLDDISVTS |
| | | AATTDIETTHPSINTVVTNVGTTGSAFESHSTVSAYPEPSKVTSP |
| | | NVTTSTMEDTTISRSIPKSSKTTRTETETTSSLTPKLRETSISQE |
| | | ITSSTETSTVPYKELTGATTEVSRTDVTSSSSTSFPGPDQSTVSL |
| | | DISTETNTRLSTSPIMTESAEITITTQTGPHGATSQDTFTMDPSN |
| | | TTPQAGIHSAMTHGFSQLDVTTLMSRIPQDVSWTSPPSVDKTSSP |
| | | SSFLSSPAMTTPSLISSTLPEDKLSSPMTSLLTSGLVKITDILRT |
| | | RLEPVTSSLPNFSSTSDKILATSKDSKDTKEIFPSINTEETNVKA |
| | | NNSGHESHSPALADSETPKATTQMVITTTVGDPAPSTSMPVHGSS |
| | | ETTNIKREPTYFLTPRLRETSTSQESSFPTDTSFLLSKVPTGTIT |
| | | EVSSTGVNSSSKISTPDHDKSTVPPDTFTGEIPRVFTSSIKTKSA |
| | | EMTITTQASPPESASHSTLPLDTSTTLSQGGTHSTVTQGFPYSEV |
| | | TTLMGMGPGNVSWMTTPPVEETSSVSSLMSSPAMTSPSPVSSTSP |
| | | QSIPSSPLPVTALPTSVLVTTTDVLGTTSPESVTSSPPNLSSITH |
| | | ERPATYKDTAHTEAAMHHSTNTAVTNVGTSGSGHKSQSSVLADSE |
| | | TSKATPLMSTTSTLGDTSVSTSTPNISQTNQIQTEPTASLSPRLR |
| | | ESSTSEKTSSTTETNTAFSYVPTGAITQASRTEISSSRTSISDLD |
| | | RPTIAPDISTGMITRLFTSPIMTKSAEMTVTTQTTTPGATSQGIL |
| | | PWDTSTTLFQGGTHSTVSQGFPHSEITTLRSRTPGDVSWMTTPPV |
| | | EETSSGFSLMSPSMTSPSPVSSTSPESIPSSPLPVTALLTSVLVT |
| | | TTNVLGTTSPEPVTSSPPNLSSPTQERLTTYKDTAHTEAMHASMH |
| | | TNTAVANVGTSISGHESQSSVPADSHTSKATSPMGITFAMGDTSV |
| | | STSTPAFFETRIQTESTSSLIPGLRDTRTSEEINTVTETSTVLSE |
| | | VPTTTTTEVSRTEVITSSRTTISGPDHSKMSPYISTETITRLSTF |
| | | PFVTGSTEMAITNQTGPIGTISQATLTLDTSSTASWEGTHSPVTQ |
| | | RFPHSEETTTMSRSTKGVSWQSPPSVEETSSPSSPVPLPAITSHS |
| | | SLYSAVSGSSPTSALPVTSLLTSGRRKTIDMLDTHSELVTSSLPS |
| | | ASSFSGEILTSEASTNTETIHFSENTAETNMGTTNSMHKLHSSVS |
| | | IHSQPSGHTPPKVTGSMMEDAIVSTSTPGSPETKNVDRDSTSPLT |
| | | PELKEDSTALVMNSTTESNTVFSSVSLDAATEVSRAEVTYYDPTF |
| | | MPASAQSTKSPDISPEASSSHSNSPPLTISTHKTIATQTGPSGVT |
| | | SLGQLTLDTSTIATSAGTPSARTQDFVDSETTSVMNNDLNDVLKT |
| | | SPFSAEEANSLSSQAPLLVTTSPSPVTSTLQEHSTSSLVSVTSVP |
| | | TPTLAKITDMDTNLEPVTRSPQNLRNTLATSEATTDTHTMHPSIN |
| | | TAVANVGTTSSPNEFYFTVSPDSDPYKATSAVVITSTSGDSIVST |
| | | SMPRSSAMKKIESETTFSLIFRLRETSTSQKIGSSSDTSTVFDKA |
| | | FTAATTEVSRTELTSSSRTSIQGTEKPTMSPDTSTRSVTMLSTFA |
| | | GLTKSEERTIATQTGPHRATSQGTLTWDTSITTSQAGTHSAMTHG |
| | | FSQLDLSTLTSRVPEYISGTSPPSVEKTSSSSSLLSLPAITSPSP |
| | | VPTTLPESRPSSPVHLTSLPTSGLVKTTDMLASVASLPPNLGSTS |
| | | HKIPTTSEDIKDTEKMYPSTNIAVTNVGTTTSEKESYSSVPAYSE |
| | | PPKVTSPMVTSFNIRDTIVSTSMPGSSEITRIEMESTFSLAHGLK |
| | | GTSTSQDPIVSTEKSAVLHKLTTGATETSRTEVASSRRTSIPGPD |
| | | HSTESPDISTEVIPSLPISLGITESSNMTIITRTGPPLGSTSQGT |
| | | FTLDTPTTSSRAGTHSMATQEFPHSEMTTVMNKDPEILSWTIPPS |
| | | IEKTSFSSSLMPSPAMTSPPVSSTLPKTIHTTPSPMTSLLTPSLV |
| | | MTTDTLGTSPEPTTSSPPNLSSTSHEILTTDEDTTAIEAMHPSTS |
| | | TAATNVETTSSGHGSQSSVLADSEKTKATAPMDTTSTMGHTTVST |
| | | SMSVSSETTKIKRESTYSLTPGLRETSISQNASFSTDTSIVLSEV |
| | | PTGTTAEVSRTEVTSSGRTSIPGPSQSTVLPEISTRTMTRLFASP |
| | | TMTESAEMTIPTQTGPSGSTSQDTLTLDTSTTKSQAKTHSTLTQR |
| | | FPHSEMTTLMSRGPGDMSWQSSPSLENPSSLPSLLSLPATTSPPP |
| | | ISSTLPVTISSSPLPVTSLLTSSPVTTTDMLHTSPELVTSSPPKL |
| | | SHTSDERLTTGKDTTNTEAVHPSTNTAASNVEIPSSGHESPSSAL |
| | | ADSETSKATSPMFITSTQEDTTVAISTPHFLETSRIQKESISSLS |
| | | PKLRETGSSVETSSAIETSAVLSEVSIGATTEISRTEVTSSSRTS |
| | | ISGSAESTMLPEISTTRKIIKFPTSPILAESSEMTIKTQTSPPGS |
| | | TSESTFTLDTSTTPSLVITHSTMTQRLPHSEITTLVSRGAGDVPR |
| | | PSSLPVEETSPPSSQLSLSAMISPSPVSSTLPASSHSSSASVTSL |
| | | LTPGQVKTTEVLDASAEPETSSPPSLSSTSVEILATSEVTTDTEK |
| | | IHPFSNTAVTKVGTSSSGHESPSSVLPDSETTKATSAMGTISIMG |
| | | DTSVSTLTPALSNTRKIQSEPASSLTTRLRETSTSEETSLATEAN |
| | | TVLSKVSTGATTEVSRTEAISFSRTSMSGPEQSTMSQDISIGTIP |
| | | RISASSVLTESAKMTITTQTGPSESTLESTLNLNTATTPSWVETH |
| | | SIVIQGFPHPEMTTSMGRGPGGVSWPSPPFVKETSPPSSPLSLPA |
| | | VTSPHPVSTTFLAHIPPSPLPVTSLLTSGPATTTDILGTSTEPGT |
| | | SSSSSLSTTSHERLTTYKDTAHTEAVHPSTNTGGTNVATTSSGYK |
| | | SQSSVLADSSPMCTTSTMGDTSVLTSTPAFLETRRIQTELASSLT |
| | | PGLRESSGSEGTSSGTKMSTVLSKVPTGATTEISKEDVTSIPGPA |
| | | QSTISPDISTRTVSWFSTSPVMTESAEITMNTHTSPLGATTQGTS |
| | | TLDTSSTTSLTMTHSTISQGFSHSQMSTLMRRGPEDVSWMSPPLL |
| | | EKTRPSFSLMSSPATTSPSPVSSTLPESISSSPLPVTSLLTSGLA |
| | | KTTDMLHKSSEPVTNSPANLSSTSVEILATSEVTTDTEKTHPSSN |
| | | RTVTDVGTSSSGHESTSFVLADSQTSKVTSPMVITSTMEDTSVST |
| | | STPGFFETSRIQTEPTSSLTLGLRKTSSSEGTSLATEMSTVLSGV |
| | | PTGATAEVSRTEVTSSSRTSISGFAQLTVSPETSTETITRLPTSS |
| | | IMTESAEMMIKTQTDPPGSTPESTHTVDISTTPNWVETHSTVTQR |
| | | FSHSEMTTLVSRSPGDMLWPSQSSVEETSSASSLLSLPATTSPSP |
| | | VSSTLVEDFPSASLPVTSLLNPGLVITTDRMGISREPGTSSTSNL |
| | | SSTSHERLTTLEDTVDTEDMQPSTHTAVTNVRTSISGHESQSSVL |
| | | SDSETPKATSPMGTTYTMGETSVSISTSDFFETSRIQIEPTSSLT |
| | | SGLRETSSSERISSATEGSTVLSEVPSGATTEVSRTEVISSRGTS |
| | | MSGPDQFTISPDISTEAITRLSTSPIMTESAESAITIETGSPGAT |
| | | SEGTLTLDTSTTTFWSGTHSTASPGFSHSEMTTLMSRTPGDVPWP |
| | | SLPSVEEASSVSSSLSSPAMTSTSFFSTLPESISSSPHPVTALLT |
| | | LGPVKTTDMLRTSSEPETSSPPNLSSTSAEILATSEVTKDREKIH |
| | | PSSNTPVVNVGTVIYKHLSPSSVLADLVTTKPTSPMATTSTLGNT |
| | | SVSTSTPAFPETMMTQPTSSLTSGLREISTSQETSSATERSASLS |
| | | GMPTGATTKVSRTEALSLGRTSTPGPAQSTISPEISTETITRIST |
| | | PLTTTGSAEMTITPKTGHSGASSQGTFTLDTSSRASWPGTHSAAT |
| | | HRSPHSGMTTPMSRGPEDVSWPSRPSVEKTSPPSSLVSLSAVTSP |
| | | SPLYSTPSESSHSSPLRVTSLFTPVMMKTTDMLDTSLEPVTTSPP |
| | | SMNITSDESLATSKATMETEAIQLSENTAVTQMGTISARQEFYSS |
| | | YPGLPEPSKVTSPVVTSSTIKDIVSTTIPASSEITRIEMESTSTL |
| | | TPTPRETSTSQEIHSATKPSTVPYKALTSATIEDSMTQVMSSSRG |
| | | PSPDQSTMSQDISTEVITRLSTSPIKTESTEMTITTQTGSPGATS |
| | | RGTLTLDTSTTFMSGTHSTASQGFSHSQMTALMSRTPGDVPWLSH |
| | | PSVEEASSASFSLSSPVMTSSSPVSSTLPDSIHSSSLPVTSLLTS |
| | | GLVKTTELLGTSSEPETSSPPNLSSTSAEILAITEVTTDTEKLEM |
| | | TNVVTSGYTHESPSSVLADSVTTKATSSMGITYPTGDTNVLTSTP |
| | | AFSDTSRIQTKSKLSLTPGLMETSISEETSSATEKSTVLSSVPTG |
| | | ATTEVSRTEAISSSRTSIPGPAQSTMSSDTSMETITRISTPLTRK |
| | | ESTDMAITPKTGPSGATSQGTFTLDSSSTASWPGTHSATTQRFPQ |
| | | SVVTTPMSRGPEDVSWPSPLSVEKNSPPSSLVSSSSVTSPSPLYS |
| | | TPSGSSHSSPVPVTSLFTSIMMKATDMLDASLEPETTSAPNMNIT |
| | | SDESLAASKATTETEAIHVFENTAASHVETTSATEELYSSSPGFS |
| | | EPTKVISPVVTSSSIRDNMVSTTMPGSSGITRIEIESMSSLTPGL |
| | | RETRTSQDITSSTETSTVLYKMPSGATPEVSRTEVMPSSRTSIPG |
| | | PAQSTMSLDISDEVVTRLSTSPIMTESAEITITTQTGYSLATSQV |
| | | TLPLGTSMTFLSGTHSTMSQGLSHSEMTNLMSRGPESLSWTSPRF |
| | | VETTRSSSSLTSLPLTTSLSPVSSTLLDSSPSSPLPVTSLILPGL |
| | | VKTTEVLDTSSEPKTSSSPNLSSTSVEIPATSEIMTDTEKIHPSS |
| | | NTAVAKVRTSSSVHESHSSVLADSETTITIPSMGITSAVDDTTVF |
| | | TSNPAFSETRRIPTEPTFSLTPGFRETSTSEETTSITETSAVLYG |
| | | VPTSATTEVSMTEIMSSNRIHIPDSDQSTMSPDIITEVITRLSSS |
| | | SMMSESTQMTITTQKSSPGATAQSTLTLATTTAPLARTHSTVPPR |
| | | FLHSEMTTLMSRSPENPSWKSSLFVEKTSSSSSLLSLPVTTSPSV |
| | | SSTLPQSIPSSSFSVTSLLTPGMVKTTDTSTEPGTSLSPNLSGTS |
| | | VEILAASEVTTDTEKIHPSSSMAVTNVGTTSSGHELYSSVSIHSE |
| | | PSKATYPVGTPSSMAETSISTSMPANFETTGFEAEPFSHLTSGFR |
| | | KTNMSLDTSSVTPTNTPSSPGSTHLLQSSKTDFTSSAKTSSPDWP |
| | | PASQYTEIPVDIITPFNASPSITESTGITSFPESRFTMSVTESTH |
| | | HLSTDLLPSAETISTGTVMPSLSEAMTSFATTGVPRAISGSGSPF |
| | | SRTESGPGDATLSTIAESLPSSTPVPFSSSTFTTTDSSTIPALHE |
| | | ITSSSATPYRVDTSLGTESSTTEGRLVMVSTLDTSSQPGRTSSSP |
| | | ILDTRMTESVELGTVTSAYQVPSLSTRLTRTDGIMEHITKIPNEA |
| | | AHRGTIRPVKGPQTSTSPASPKGLHTGGTKRMETTTTALKTTTTA |
| | | LKTTSRATLTTSVYTPTLGTLTPLNASMQMASTIPTEMMITTPYV |
| | | FPDVPETTSSLATSLGAETSTALPRTTPSVFNRESETTASLVSRS |
| | | GAERSPVIQTLDVSSSEPDTTASWVIHPAETIPTVSKTTPNFFHS |
| | | ELDTVSSTATSHGADVSSAIPTNISPSELDALTPLVTISGTDTST |
| | | TFPTLTKSPHETETRTTWLTHPAETSSTIPRTIPNFSHHESDATP |
| | | SIATSPGAETSSAIPIMTVSPGAEDLVTSQVTSSGTDRNMTIPTL |
| | | TLSPGEPKTIASLVTHPEAQTSSAIPTSTISPAVSRLVTSMVTSL |
| | | AAKTSTTNRALTNSPGEPATTVSLVTHPAQTSPTVPWTTSIFFHS |
| | | KSDTTPSMTTSHGAESSSAVPTPTVSTEVPGVVTPLVTSSRAVIS |
| | | TTIPILTLSPGEPETTPSMATSHGEEASSAIPTPTVSPGVPGVVT |
| | | SLVTSSRAVTSTTIPILTFSLGEPETTPSMATSHGTEAGSAVPTV |
| | | LPEVPGMVTSLVASSRAVTSTTLPTLTLSPGEPETTPSMATSHGA |
| | | EASSTVPTVSPEVPGVVTSLVTSSSGVNSTSIPTLILSPGELETT |
| | | PSMATSHGAEASSAVPTPTVSPGVSGVVTPLVTSSRAVTSTTIPI |
| | | LTLSSSEPETTPSMATSHGVEASSAVLTVSPEVPGMVTSLVTSSR |
| | | AVTSTTIPTLTISSDEPETTTSLVTHSEAKMISAIPTLAVSPTVQ |
| | | GLVTSLVTSSGSETSAFSNITVASSQPETIDSWVAHPGTEASSVV |
| | | PTLTVSTGEPFTNISLVTHPAESSSTLPRTTSRFSHSELDTMPST |
| | | VTSPEAESSSAISTTISPGIPGVLTSLVTSSGRDISATFPTVPES |
| | | PHESEATASWVTHPAVTSTTVPRTTPNYSHSEPDTTPSIATSPGA |
| | | EATSDFPTITVSPDVPDMVTSQVTSSGTDTSITIPTLTLSSGEPE |
| | | TTTSFITYSETHTSSAIPTLPVSPGASKMLTSLVISSGTDSTTTF |
| | | PTLTETPYEPETTAIQLIHPAETNTMVPRTTPKFSHSKSDTTLPV |
| | | AITSPGPEASSAVSTTTISPDMSDLVTSLVPSSGTDTSTTFPTLS |
| | | ETPYEPETTATWLTHPAETSTTVSGTIPNFSHRGSDTAPSMVTSP |
| | | GVDTRSGVPTTTIPPSIPGVVTSQVTSSATDTSTAIPTLTPSPGE |
| | | PETTASSATHPGTQTGFTVPIRTVPSSEPDTMASWVTHPPQTSTP |
| | | VSRTTSSFSHSSPDATPVMATSPRTEASSAVLTTISPGAPEMVTS |
| | | QITSSGAATSTTVPTLTHSPGMPETTALLSTHPRTETSKTFPAST |
| | | VFPQVSETTASLTIRPGAETSTALPTQTTSSLFTLLVTGTSRVDL |
| | | SPTASPGVSAKTAPLSTHPGTETSTMIPTSTLSLGLLETTGLLAT |
| | | SSSAETSTSTLTLTVSPAVSGLSSASITTDKPQTVTSWNTETSPS |
| | | VTSVGPPEFSRTVTGTTMTLIPSEMPTPPKTSHGEGVSPTTILRT |
| | | TMVEATNLATTGSSPTVAKTTTTFNTLAGSLFTPLTTPGMSTLAS |
| | | ESVTSRTSYNHRSWISTTSSYNRRYWTPATSTPVTSTFSPGISTS |
| | | SIPSSTAATVPFMVPFTLNFTITNLQYEEDMRHPGSRKFNATERE |
| | | LQGLLKPLFRNSSLEYLYSGCRLASLRPEKDSSATAVDAICTHRP |
| | | DPEDLGLDRERLYWELSNLTNGIQELGPYTLDRNSLYVNGFTHRS |
| | | SMPTTSTPGTSTVDVGTSGTPSSSPSPTTAGPLLMPFTLNFTITN |
| | | LQYEEDMRRTGSRKFNTMESVLQGLLKPLFKNTSVGPLYSGCRLT |
| | | LLRPEKDGAATGVDAICTHRLDPKSPGLNREQLYWELSKLINDIE |
| | | ELGPYTLDRNSLYVNGFTHQSSVSTTSTPGTSTVDLRTSGTPSSL |
| | | SSPTIMAAGPLLVPFTLNFTITNLQYGEDMGHPGSRKFNTTERVL |
| | | QGLLGPIFKNTSVGPLYSGCRLTSLRSEKDGAATGVDAICIHHLD |
| | | PKSPGLNRERLYWELSQLTNGIKELGPYTLDRNSLYVNGFTHRTS |
| | | VPTSSTPGTSTVDLGTSGTPFSLPSPATAGPLLVLFTLNFTITNL |
| | | KYEEDMHRPGSRKFNTTERVLQTLLGPMFKNTSVGLLYSGCRLTL |
| | | LRSEKDGAATGVDAICTHRLDPKSPGVDREQLYWELSQLTNGIKE |
| | | LGPYTLDRNSLYVNGFTHWIPVPTSSTPGTSTVDLGSGTPSSLPS |
| | | PTTAGPLLVPFTLNFTITNLKYEEDMHCPGSRKFNTTERVLQSLL |
| | | GPMFKNTSVGPLYSGCRLTLLRSEKDGAATGVDAICTHRLDPKSP |
| | | GVDREQLYWELSQLTNGIKELGPYTLDRNSLYVNGFTHQTSAPNT |
| | | STPGTSTVDLGTSGTPSSLPSPTSAGPLLVPFTLNFTITNLQYEE |
| | | DMHHPGSRKFNTTERVLQGLLGPMFKNTSVGLLYSGCRLTLLRPE |
| | | KNGAATGMDAICSHRLDPKSPGLNREQLYWELSQLTHGIKELGPY |
| | | TLDRNSLYVNGFTHRSSVAPTSTPGTSTVDLGTSGTPSSLPSPTT |
| | | AVPLLVPFTLNFTITNLQYGEDMRHPGSRKFNTTERVLQGLLGPL |
| | | FKNSSVGPLYSGCRLISLRSEKDGAATGVDAICTHHLNPQSPGLD |
| | | REQLYWQLSQMTNGIKELGPYTLDRNSLYVNGFTHRSSGLTTSTP |
| | | WTSTVDLGTSGTPSPVPSPTTTGPLLVPFTLNFTITNLQYEENMG |
| | | HPGSRKFNITESVLQGLLKPLFKSTSVGPLYSGCRLTLLRPEKDG |
| | | VATRVDAICTHRPDPKIPGLDRQQLYWELSQLTHSITELGPYTLD |
| | | RDSLYVNGFTQRSSVPTTSTPGTFTVQPETSETPSSLPGPTATGP |
| | | VLLPFTLNFTITNLQYEEDMRRPGSRKFNTTERVLQGLLMPLFKN |
| | | TSVSSLYSGCRLTLLRPEKDGAATRVDAVCTHRPDPKSPGLDRER |
| | | LYWKLSQLTHGITELGPYTLDRHSLYVNGFTHQSSMTTTRTPDTS |
| | | TMHLATSRTPASLSGPMTASPLLVLFTINFTITNLRYEENMHHPG |
| | | SRKFNTTERVLQGLLRPVFKNTSVGPLYSGCRLTLLRPKKDGAAT |
| | | KVDAICTYRPDPKSPGLDREQLYWELSQLTHSITELGPYTLDRDS |
| | | LYVNGFTQRSSVPTTSIPGTPTVDLGTSGTPVSKPGPSAASPLLV |
| | | LFTLNFTITNLRYEENMQHPGSRKFNTTERVLQGLLRSLFKSTSV |
| | | GPLYSGCRLTLLRPEKDGTATGVDAICTHHPDPKSPRLDREQLYW |
| | | ELSQLTHNITELGPYALDNDSLFVNGFTHRSSVSTTSTPGTPTVY |
| | | LGASKTPASIFGPSAASHLLILFTLNFTITNLRYEENMWPGSRKF |
| | | NTTERVLQGLLRPLFKNTSVGPLYSGCRLTLLRPEKDGEATGVDA |
| | | ICTHRPDPTGPGLDREQLYLELSQLTHSITELGPYTLDRDSLYVN |
| | | GFTHRSSVPTTSTGVVSEEPFTLNFTINNLRYMADMGQPGSLKFN |
| | | ITDNVMQHLLSPLFQRSSLGARYTGCRVIALRSVKNGAETRVDLL |
| | | CTYLQPLSGPGLPIKQVFHELSQQTHGITRLGPYSLDKDSLYLNG |
| | | YNEPGPDEPPTTPKPATTFLPPLSEATTAMGYHLKTLTLNFTISN |
| | | LQYSPDMGKGSATFNSTEGVLQHLLRPLFQKSSMGPFYLGCQLIS |
| | | LRPEKDGAATGVDTTCTYHPDPVGPGLDIQQLYWELSQLTHGVTQ |
| | | LGFYVLDRDSLFINGYAPQNLSIRGEYQINFHIVNWNLSNPDPTS |
| | | SEYITLLRDIQDKVTTLYKGSQLHDTFRFCLVTNLTMDSVLVTVK |
| | | ALFSSNLDPSLVEQVFLDKTLNASFHWLGSTYQLVDIHVTEMESS |
| | | VYQPTSSSSTQHFYLNFTITNLPYSQDKAQPGTTNYQRNKRNIED |
| | | ALNQLFRNSSIKSYFSDCQVSTFRSVPNRHHTGVDSLCNFSPLAR |
| | | RVDRVAIYEEFLRMTRNGTQLQNFTLDRSSVLVDGYSPNRNEPLT |
| | | GNSDLPFWAVILIGLAGLLGVITCLICGVLVTTRRRKKEGEYNVQ |
| | | QQCPGYYQSHLDLEDLQ |
| SEZ6 | 231 | MRPVALLLLPSLLALLAHGLSLEAPTVGKGQAPGIEETDGELTAA |
| | | PTPEQPERGVHFVTTAPTLKLLNHHPLLEEFLQEGLEKGDEELRP |
| | | ALPFQPDPPAPFTPSPLPRLANQDSRPVFTSPTPAMAAVPTQPQS |
| | | KEGPWSPESESPMLRITAPLPPGPSMAVPTLGPGEIASTTPPSRA |
| | | WTPTQEGPGDMGRPWVAEVVSQGAGIGIQGTITSSTASGDDEETT |
| | | TTTTIITTTITTVQTPGPCSWNFSGPEGSLDSPTDLSSPTDVGLD |
| | | CFFYISVYPGYGVEIKVQNISLREGETVTVEGLGGPDPLPLANQS |
| | | FLLRGQVIRSPTHQAALRFQSLPPPAGPGTFHFHYQAYLLSCHFP |
| | | RRPAYGDVTVTSLHPGGSARFHCATGYQLKGARHLTCLNATQPFW |
| | | DSKEPVCIAACGGVIRNATTGRIVSPGFPGNYSNNLTCHWLLEAP |
| | | EGQRLHLHFEKVSLAEDDDRLIIRNGDNVEAPPVYDSYEVEYLPI |
| | | EGLLSSGKHFFVELSTDSSGAAAGMALRYEAFQQGHCYEPFVKYG |
| | | NFSSSTPTYPVGTTVEFSCDPGYTLEQGSIIIECVDPHDPQWNET |
| | | EPACRAVCSGEITDSAGVVLSPNWPEPYGRGQDCIWGVHVEEDKR |
| | | IMLDIRVLRIGPGDVLTFYDGDDLTARVLGQYSGPRSHFKLFTSM |
| | | ADVTIQFQSDPGTSVLGYQQGFVIHFFEVPRNDTCPELPEIPNGW |
| | | KSPSQPELVHGTVVTYQCYPGYQVVGSSVLMCQWDLTWSEDLPSC |
| | | QRVTSCHDPGDVEHSRRLISSPKFPVGATVQYICDQGFVLMGSSI |
| | | LTCHDRQAGSPKWSDRAPKCLLEQLKPCHGLSAPENGARSPEKQL |
| | | HPAGATIHFSCAPGYVLKGQASIKCVPGHPSHWSDPPPICRAASL |
| | | DGFYNSRSLDVAKAPAASSTLDAAHIAAAIFLPLVAMVLLVGGVY |
| | | FYFSRLQGKSSLQLPRPRPRPYNRITIESAFDNPTYETGSLSFAG |
| | | DERI |
| CD56 | 232 | MLQTKDLIWTLFFLGTAVSLQVDIVPSQGEISVGESKFFLCQVAG |
| | | DAKDKDISWFSPNGEKLTPNQQRISVVWNDDSSSTLTIYNANIDD |
| | | AGIYKCVVTGEDGSESEATVNVKIFQKLMFKNAPTPQEFREGEDA |
| | | VIVCDVVSSLPPTIIWKHKGRDVILKKDVRFIVLSNNYLQIRGIK |
| | | KTDEGTYRCEGRILARGEINFKDIQVIVNVPPTIQARQNIVNATA |
| | | NLGQSVTLVCDAEGFPEPTMSWTKDGEQIEQEEDDEKYIFSDDSS |
| | | QLTIKKVDKNDEAEYICIAENKAGEQDATIHLKVFAKPKITYVEN |
| | | QTAMELEEQVTLTCEASGDPIPSITWRTSTRNISSEEKASWTRPE |
| | | KQETLDGHMVVRSHARVSSLTLKSIQYTDAGEYICTASNTIGQDS |
| | | QSMYLEVQYAPKLQGPVAVYTWEGNQVNITCEVFAYPSATISWFR |
| | | DGQLLPSSNYSNIKIYNTPSASYLEVTPDSENDFGNYNCTAVNRI |
| | | GQESLEFILVQADTPSSPSIDQVEPYSSTAQVQFDEPEATGGVPI |
| | | LKYKAEWRAVGEEVWHSKWYDAKEASMEGIVTIVGLKPETTYAVR |
| | | LAALNGKGLGEISAASEFKTQPVQGEPSAPKLEGQMGEDGNSIKV |
| | | NLIKQDDGGSPIRHYLVRYRALSSEWKPEIRLPSGSDHVMLKSLD |
| | | WNAEYEVYVVAENQQGKSKAAHFVFRTSAQPTAIPANGSPTSGLS |
| | | TGAIVGILIVIFVLLLVVVDITCYFLNKCGLFMCIAVNLCGKAGP |
| | | GAKGKDMEEGKAAFSKDESKEPIVEVRTEEERTPNHDGGKHTEPN |
| | | ETTPLTEPEKGPVEAKPECQETETKPAPAEVKTVPNDATQTKENE |
| | | SKA |
| DLL3 | 233 | MVSPRMSGLLSQTVILALIFLPQTRPAGVFELQIHSFGPGPGPGA |
| | | PRSPCSARLPCRLFFRVCLKPGLSEEAAESPCALGAALSARGPVY |
| | | TEQPGAPAPDLPLPDGLLQVPFRDAWPGTFSFIIETWREELGDQI |
| | | GGPAWSLLARVAGRRRLAAGGPWARDIQRAGAWELRFSYRARCEP |
| | | PAVGTACTRLCRPRSAPSRCGPGLRPCAPLEDECEAPLVCRAGCS |
| | | PEHGFCEQPGECRCLEGWTGPLCTVPVSTSSCLSPRGPSSATTGC |
| | | LVPGPGPCDGNPCANGGSCSETPRSFECTCPRGFYGLRCEVSGVT |
| | | CADGPCFNGGLCVGGADPDSAYICHCPPGFQGSNCEKRVDRCSLQ |
| | | PCRNGGLCLDLGHALRCRCRAGFAGPRCEHDLDDCAGRACANGGT |
| | | CVEGGGAHRCSCALGFGGRDCRERADPCAARPCAHGGRCYAHFSG |
| | | LVCACAPGYMGARCEFPVHPDGASALPAAPPGLRPGDPQRYLLPP |
| | | ALGLLVAAGVAGAALLLVHVRRRGHSQDAGSRLLAGTPEPSVHAL |
| | | PDALNNLRTQEGSGDGPSSSVDWNRPEDVDPQGIYVISAPSIYAR |
| | | EVATPLFPPLHTGRAGQRQHLLFPYPSSILSVK |
| DLK1 | 234 | MTATEALLRVLLLLLAFGHSTYGAECFPACNPQNGFCEDDNVCRC |
| | | QPGWQGPLCDQCVTSPGCLHGLCGEPGQCICTDGWDGELCDRDVR |
| | | ACSSAPCANNRTCVSLDDGLYECSCAPGYSGKDCQKKDGPCVING |
| | | SPCQHGGTCVDDEGRASHASCLCPPGFSGNFCEIVANSCTPNPCE |
| | | NDGVCTDIGGDFRCRCPAGFIDKTCSRPVINCASSPCQNGGTCLQ |
| | | HTQVSYECLCKPEFTGLTCVKKRALSPQQVTRLPSGYGLAYRLTP |
| | | GVHELPVQQPEHRILKVSMKELNKKTPLLTEGQAICFTILGVLTS |
| | | LVVLGTVGIVFLNKCETWVSNLRYNHMLRKKKNLLLQYNSGEDLA |
| | | VNIIFPEKIDMTTFSKEAGDEEI |
| B7H3 | 79 | MLRRRGSPGMGVHVGAALGALWFCLTGALEVQVPEDPVVALVGTD |
| | | ATLCCSFSPEPGFSLAQLNLIWQLTDTKQLVHSFAEGQDQGSAYA |
| | | NRTALFPDLLAQGNASLRLQRVRVADEGSFTCFVSIRDFGSAAVS |
| | | LQVAAPYSKPSMTLEPNKDLRPGDTVTITCSSYQGYPEAEVFWQD |
| | | GQGVPLTGNVTTSQMANEQGLFDVHSILRVVLGANGTYSCLVRNP |
| | | VLQQDAHSSVTITPQRSPTGAVEVQVPEDPVVALVGTDATLRCSF |
| | | SPEPGFSLAQLNLIWQLTDTKQLVHSFTEGRDQGSAYANRTALFP |
| | | DLLAQGNASLRLQRVRVADEGSFTCFVSIRDFGSAAVSLQVAAPY |
| | | SKPSMTLEPNKDLRPGDTVTITCSSYRGYPEAEVFWQDGQGVPLT |
| | | GNVTTSQMANEQGLFDVHSVLRVVLGANGTYSCLVRNPVLQQDAH |
| | | GSVTITGQPMTFPPEALWVTVGLSVCLIALLVALAFVCWRKIKQS |
| | | CEEENAGAEDQDGEGEGSKTALQPLKHSDSKEDDGQEIA |
| EphA2 | 337 | MELQAARACFALLWGCALAAAAAAQGKEVVLLDFAAAGGELGWLT |
| | | HPYGKGWDLMQNIMNDMPIYMYSVCNVMSGDQDNWLRTNWVYRGE |
| | | AERIFIELKFTVRDCNSFPGGASSCKETFNLYYAESDLDYGTNFQ |
| | | KRLFTKIDTIAPDEITVSSDFEARHVKLNVEERSVGPLTRKGFYL |
| | | AFQDIGACVALLSVRVYYKKCPELLQGLAHFPETIAGSDAPSLAT |
| | | VAGTCVDHAVVPPGGEEPRMHCAVDGEWLVPIGQCLCQAGYEKVE |
| | | DACQACSPGFFKFEASESPCLECPEHTLPSPEGATSCECEEGFFR |
| | | APQDPASMPCTRPPSAPHYLTAVGMGAKVELRWTPPQDSGGREDI |
| | | VYSVTCEQCWPESGECGPCEASVRYSEPPHGLTRTSVTVSDLEPH |
| | | MNYTFTVEARNGVSGLVTSRSFRTASVSINQTEPPKVRLEGRSTT |
| | | SLSVSWSIPPPQQSRVWKYEVTYRKKGDSNSYNVRRTEGFSVTLD |
| | | DLAPDTTYLVQVQALTQEGQGAGSKVHEFQTLSPEGSGNLAVIGG |
| | | VAVGVVLLLVLAGVGFFIHRRRKNQRARQSPEDVYFSKSEQLKPL |
| | | KTYVDPHTYEDPNQAVLKFTTEIHPSCVTRQKVIGAGEFGEVYKG |
| | | MLKTSSGKKEVPVAIKTLKAGYTEKQRVDFLGEAGIMGQFSHHNI |
| | | IRLEGVISKYKPMMIITEYMENGALDKFLREKDGEFSVLQLVGML |
| | | RGIAAGMKYLANMNYVHRDLAARNILVNSNLVCKVSDFGLSRVLE |
| | | DDPEATYTTSGGKIPIRWTAPEAISYRKFTSASDVWSFGIVMWEV |
| | | MTYGERPYWELSNHEVMKAINDGFRLPTPMDCPSAIYQLMMQCWQ |
| | | QERARRPKFADIVSILDKLIRAPDSLKTLADFDPRVSIRLPSTSG |
| | | SEGVPFRTVSEWLESIKMQQYTEHFMAAGYTAIEKVVQMINDDIK |
| | | RIGVRLPGHQKRIAYSLLGLKDQVNTVGIPI |

TABLE D7
Exemplary Antibody Sequences

| Ab | SEQ ID NO | IgG chain | Amino acid/Nucleotide sequence |
|---|---|---|---|
| CD74 | 118 | Heavy | QVQLQQSGSELKKPGASVKVSCKASGYTFTNYGVNWIK |
| milatuzumab | | Chain (Wild | QAPGQGLQWMGWINPNTGEPTFDDDFKGRFAFSLDTSV |
| Cysmab HC | | Type Fc) | STAYLQISSLKADDTAVYFCSRSRGKNEAWFAYWGQGT |
| (VHmil x | | | LVTVSSASTKGPSVFPLAPSSKSTSGGTAALGCLVKDY |
| VKlaNQ) | | | FPCPVTVSWNSGALTSGVHTFPAVLQSSGLYSLSSVVT |
| | | | VPSSSLGTQTYICNVNHKPSNTKVDKRVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPCDI |
| | | | AVEWESNGQPENNYKTTPPVLDSDGSFFLYSKLTVDKS |
| | | | RWQQGNVFSCSVMHEALHNHYTQKSLSLSPGK |
| | 235 | DNA Heavy | CAGGTTCAGCTCCAGCAGTCTGGCAGCGAGCTGAAAAA |
| | | Chain (Wild | ACCTGGCGCCTCCGTGAAGGTGTCCTGCAAGGCTTCTG |
| | | Type Fc) | GCTACACCTTTACCAACTACGGCGTGAACTGGATCAAG |
| | | | CAGGCCCCTGGACAAGGCCTCCAATGGATGGGCTGGAT |
| | | | CAACCCCAATACCGGCGAGCCCACCTTCGACGACGATT |
| | | | TCAAGGGCAGATTCGCCTTCAGCCTGGACACCTCTGTG |
| | | | TCCACAGCCTACCTCCAGATCAGCAGCCTGAAGGCCGA |
| | | | TGATACCGCCGTGTACTTCTGCTCCAGAAGCCGGGGAA |
| | | | AGAACGAGGCTTGGTTTGCCTATTGGGGCCAGGGCACA |
| | | | CTGGTCACCGTTAGCTCTGCTAGCACCAAGGGCCCAAG |
| | | | TGTGTTTCCCCTGGCCCCCAGCAGCAAGTCTACTTCCG |
| | | | GCGGAACTGCTGCCCTGGGTTGCCTGGTGAAGGACTAC |
| | | | TTCCCCTGTCCCGTGACAGTGTCCTGGAACTCTGGGGC |
| | | | TCTGACTTCCGGCGTGCACACCTTCCCCGCCGTGCTGC |
| | | | AGAGCAGCGGCCTGTACAGCCTGAGCAGCGTGGTGACA |
| | | | GTGCCCTCCAGCTCTCTGGGAACCCAGACCTATATCTG |
| | | | CAACGTGAACCACAAGCCCAGCAACACCAAGGTGGACA |
| | | | AGAGAGTGGAGCCCAAGAGCTGCGACAAGACCCACACC |
| | | | TGCCCCCCCTGCCCAGCTCCAGAACTGCTGGGAGGGCC |
| | | | TTCCGTGTTCCTGTTCCCCCCCAAGCCCAAGGACACCC |
| | | | TGATGATCAGCAGGACCCCCGAGGTGACCTGCGTGGTG |
| | | | GTGGACGTGTCCCACGAGGACCCAGAGGTGAAGTTCAA |
| | | | CTGGTACGTGGACGGCGTGGAGGTGCACAACGCCAAGA |
| | | | CCAAGCCCAGAGAGGAGCAGTACAACAGCACCTACAGG |
| | | | GTGGTGTCCGTGCTGACCGTGCTGCACCAGGACTGGCT |
| | | | GAACGGCAAAGAATACAAGTGCAAAGTCTCCAACAAGG |
| | | | CCCTGCCAGCCCCAATCGAAAAGACAATCAGCAAGGCC |
| | | | AAGGGCCAGCCACGGGAGCCCCAGGTGTACACCCTGCC |
| | | | CCCCAGCCGGGAGGAGATGACCAAGAACCAGGTGTCCC |
| | | | TGACCTGTCTGGTGAAGGGCTTCTACCCCTGTGATATC |
| | | | GCCGTGGAGTGGGAGAGCAACGGCCAGCCCGAGAACAA |
| | | | CTACAAGACCACCCCCCCAGTGCTGGACAGCGACGGCA |
| | | | GCTTCTTCCTGTACAGCAAGCTGACCGTGGACAAGTCC |
| | | | AGGTGGCAGCAGGGCAACGTGTTCAGCTGCAGCGTGAT |
| | | | GCACGAGGCCCTGCACAACCACTACACCCAGAAGTCCC |
| | | | TGAGCCTGAGCCCCGGCAAG |
| CD74 | 236 | Heavy | QVQLQQSGSELKKPGASVKVSCKASGYTFTNYGVNWIK |
| Milatuzumab | | Chain (Fc | QAPGQGLQWMGWINPNTGEPTFDDDFKGRFAFSLDTSV |
| DANAPA | | Silenced | STAYLQISSLKADDTAVYFCSRSRGKNEAWFAYWGQGT |
| Cysmab | | DANAPA) | LVTVSSASTKGPSVFPLAPSSKSTSGGTAALGCLVKDY |
| | | | FPCPVTVSWNSGALTSGVHTFPAVLQSSGLYSLSSVVT |
| | | | VPSSSLGTQTYICNVNHKPSNTKVDKRVEPKSCDKTHT |
| | | | CPPCPAPELLGGPSVFLFPPKPKDTLMISRTPEVTCVV |
| | | | VAVSHEDPEVKFNWYVDGVEVHNAKTKPREEQYASTYR |
| | | | VVSVLTVLHQDWLNGKEYKCKVSNKALAAPIEKTISKA |
| | | | KGQPREPQVYTLPPSREEMTKNQVSLTCLVKGFYPCDI |
| | | | AVEWESNGQPENNYKTTPPVLDSDGSFFLYSKLTVDKS |
| | | | RWQQGNVFSCSVMHEALHNHYTQKSLSLSPGK |
| CD74 | 237 | Light Chain | DIQLTQSPLSLPVTLGQPASISCRSSQSLVHRNGNTYL |
| Milatuzumab | | | HWFQQRPGQSPRLLIYTVSNRFSGVPDRFSGSGSGTDF |
| LC | | | TLKISRVEAEDVGVYFCSQSSHVPPTFGAGTRLEIKRT |
| | | | VAAPSVFIFPPSDEQLKSGTASVVCLLNNFYPREAKVQ |
| | | | WKVDNALQSGNSQESVTEQDSKDSTYSLSSTLTLSKAD |
| | | | YEKHKVYACEVTHQGLSSPVTKSFNRGEC |
| | 238 | DNA Light | GACATTCAGCTGACACAGAGCCCTCTGAGCCTGCCTGT |
| | | Chain | TACACTGGGACAGCCTGCCAGCATCAGCTGTAGAAGCA |
| | | | GCCAGAGCCTGGTGCACAGAAACGGCAACACCTACCTG |
| | | | CACTGGTTCCAGCAGAGGCCTGGCCAGTCTCCTAGACT |
| | | | GCTGATCTACACCGTGTCCAACAGATTCAGCGGCGTGC |
| | | | CCGATAGATTTTCCGGCAGCGGCTCTGGCACCGACTTC |
| | | | ACCCTGAAGATTAGCAGAGTGGAAGCCGAGGACGTGGG |
| | | | CGTGTACTTCTGTAGCCAGTCTAGCCACGTGCCACCTA |
| | | | CCTTTGGCGCCGGAACCAGACTGGAAATCAAGCGTACG |
| | | | GTGGCCGCTCCCAGCGTGTTCATCTTCCCCCCCAGCGA |
| | | | CGAGCAGCTGAAGAGTGGCACCGCCAGCGTGGTGTGCC |
| | | | TGCTGAACAACTTCTACCCCCGGGAGGCCAAGGTGCAG |
| | | | TGGAAGGTGGACAACGCCCTGCAGAGCGGCAACAGCCA |
| | | | GGAGAGCGTCACCGAGCAGGACAGCAAGGACTCCACCT |
| | | | ACAGCCTGAGCAGCACCCTGACCCTGAGCAAGGCCGAC |
| | | | TACGAGAAGCATAAGGTGTACGCCTGCGAGGTGACCCA |
| | | | CCAGGGCCTGTCCAGCCCCGTGACCAAGAGCTTCAACA |
| | | | GGGGCGAGTGC |
| CD74 LC | 239 | Light Chain | DIVMTQTPLSLPVTPGEPASISCRSSQSLVHRNQNTYL |
| Cysmab WT | | | HWYLQKPGQSPQLLIYTVSNRFSGVPDRFSGSGSGTDF |
| (VHmil x | | | TLKSRVEAEDVGVYFCSQSSHVPPTFGQGTKLEIKRTV |
| VKlaNQ) | | | AAPSVFIFPPSDEQLKSGTASVVCLLNNFYPREAKVQW |
| | | | KVDNALSGNSQESVTEQDSKDSTYSLSSTLTLSKADYE |
| | | | KHKVYACEVTHQGLSSPVTKSFNRGEC |
| CD48 NY920 | 240 | Heavy | EVQLLESGGGLVQPGGSLRLSCAASGFTFSSFAMSWVR |
| Cysmab WT | | Chain (Wild | QAPGKGLEWVSAISGFGGSTYYADSVKGRFTISRDNSK |
| HC | | Type Fc) | NTLYLQMNSLRAEDTAVYYCARQFWEDQPFYFDYWGQG |
| | | | TLVTVSSASTKGPSVFPLAPSSKSTSGGTAALGCLVKD |
| | | | YFPCPVTVSWNSGALTSGVHTFPAVLQSSGLYSLSSVV |
| | | | TVPSSSLGTQTYICNVNHKPSNTKVDKRVEPKSCDKTH |
| | | | TCPPCPAPELLGGPSVFLFPPKPKDTLMISRTPEVTCV |
| | | | VVDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSREEMTKNQVSLTCLVKGFYPCD |
| | | | IAVEWESNGQPENNYKTTPPVLDSDGSFFLYSKLTVDK |
| | | | SRWQQGNVFSCSVMHEALHNHYTQKSLSLSPGK |
| | 241 | DNA Heavy | GAGGTGCAGCTGCTGGAGAGCGGCGGCGGCCTGGTGCA |
| | | Chain | GCCCGGCGGCAGCCTGCGGCTGAGCTGCGCCGCCAGCG |
| | | | GCTTCACCTTCAGCAGCTTCGCCATGAGCTGGGTGCGG |
| | | | CAGGCCCCCGGCAAGGGCCTGGAGTGGGTGAGCGCCAT |
| | | | CAGCGGCTTCGGCGGCAGCACCTACTACGCCGACAGCG |
| | | | TGAAGGGCCGGTTCACCATCAGCCGGGACAACAGCAAG |
| | | | AACACCCTGTACCTGCAGATGAACAGCCTGCGGGCCGA |
| | | | GGACACCGCCGTGTACTACTGCGCCCGGCAGTTCTGGG |
| | | | AGGACCAGCCCTTCTACTTCGACTACTGGGGCCAGGGC |
| | | | ACCCTGGTGACCGTGAGCAGCGCCAGCACCAAGGGCCC |
| | | | CAGCGTGTTCCCCCTGGCCCCCAGCAGCAAGAGCACCA |
| | | | GCGGCGGCACCGCCGCCCTGGGCTGCCTGGTGAAGGAC |
| | | | TACTTCCCCTGCCCCGTGACCGTGAGCTGGAACAGCGG |
| | | | CGCCCTGACCAGCGGCGTGCACACCTTCCCCGCCGTGC |
| | | | TGCAGAGCAGCGGCCTGTACAGCCTGAGCAGCGTGGTG |
| | | | ACCGTGCCCAGCAGCAGCCTGGGCACCCAGACCTACAT |
| | | | CTGCAACGTGAACCACAAGCCCAGCAACACCAAGGTGG |
| | | | ACAAGCGGGTGGAGCCCAAGAGCTGCGACAAGACCCAC |
| | | | ACCTGCCCCCCCTGCCCCGCCCCCGAGCTGCTGGGCGG |
| | | | CCCCAGCGTGTTCCTGTTCCCCCCCAAGCCCAAGGACA |
| | | | CCCTGATGATCAGCCGGACCCCCGAGGTGACCTGCGTG |
| | | | GTGGTGGACGTGAGCCACGAGGACCCCGAGGTGAAGTT |
| | | | CAACTGGTACGTGGACGGCGTGGAGGTGCACAACGCCA |
| | | | AGACCAAGCCCCGGGAGGAGCAGTACAACAGCACCTAC |
| | | | CGGGTGGTGAGCGTGCTGACCGTGCTGCACCAGGACTG |
| | | | GCTGAACGGCAAGGAGTACAAGTGCAAGGTGAGCAACA |
| | | | AGGCCCTGCCCGCCCCCATCGAGAAGACCATCAGCAAG |
| | | | GCCAAGGGCCAGCCCCGGGAGCCCCAGGTGTACACCCT |
| | | | GCCCCCCAGCCGGGAGGAGATGACCAAGAACCAGGTGA |
| | | | GCCTGACCTGCCTGGTGAAGGGCTTCTACCCCTGCGAC |
| | | | ATCGCCGTGGAGTGGGAGAGCAACGGCCAGCCCGAGAA |
| | | | CAACTACAAGACCACCCCCCCCGTGCTGGACAGCGACG |
| | | | GCAGCTTCTTCCTGTACAGCAAGCTGACCGTGGACAAG |
| | | | AGCCGGTGGCAGCAGGGCAACGTGTTCAGCTGCAGCGT |
| | | | GATGCACGAGGCCCTGCACAACCACTACACCCAGAAGA |
| | | | GCCTGAGCCTGAGCCCCGGCAAG |
| CD48 NY920 | 242 | | EVQLLESGGGLVQPGGSLRLSCAASGFTFSSFAMSWVR |
| Cysmab DAPA | | | QAPGKGLEWVSAISGFGGSTYYADSVKGRFTISRDNSK |
| HC | | | NTLYLQMNSLRAEDTAVYYCARQFWEDQPFYFDYWGQG |
| | | | TLVTVSSASTKGPSVFPLAPSSKSTSGGTAALGCLVKD |
| | | | YFPCPVTVSWNSGALTSGVHTFPAVLQSSGLYSLSSVV |
| | | | TVPSSSLGTQTYICNVNHKPSNTKVDKRVEPKSCDKTH |
| | | | TCPPCPAPELLGGPSVFLFPPKPKDTLMISRTPEVTCV |
| | | | VVAVSHEDPEVKFNWYVDGVEVHNAKTKPREEQYNSTY |
| | | | RVVSVLTVLHQDWLNGKEYKCKVSNKALAAPIEKTISK |
| | | | AKGQPREPQVYTLPPSREEMTKNQVSLTCLVKGFYPCD |
| | | | IAVEWESNGQPENNYKTTPPVLDSDGSFFLYSKLTVDK |
| | | | SRWQQGNVFSCSVMHEALHNHYTQKSLSLSPGK |
| CD48 NY920 | 243 | Light Chain | DIQMTQSPSSLSASVGDRVTITCRASQSISSYLNWYQQ |
| LC | | | KPGKAPKLLIYAASSLQSGVPSRFSGSGSGTDFTLTIS |
| | | | SLQPEDFATYYCQQSYSTPLTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 244 | DNA Light | GACATCCAGATGACCCAGAGCCCCAGCAGCCTGAGCGC |
| | | Chain | CAGCGTGGGCGACCGGGTGACCATCACCTGCCGGGCCA |
| | | | GCCAGAGCATCAGCAGCTACCTGAACTGGTACCAGCAG |
| | | | AAGCCCGGCAAGGCCCCCAAGCTGCTGATCTACGCCGC |
| | | | CAGCAGCCTGCAGAGCGGCGTGCCCAGCCGGTTCAGCG |
| | | | GCAGCGGCAGCGGCACCGACTTCACCCTGACCATCAGC |
| | | | AGCCTGCAGCCCGAGGACTTCGCCACCTACTACTGCCA |
| | | | GCAGAGCTACAGCACCCCCCTGACCTTCGGCCAGGGCA |
| | | | CCAAGGTGGAGATCAAGCGGACCGTGGCCGCCCCCAGC |
| | | | GTGTTCATCTTCCCCCCCAGCGACGAGCAGCTGAAGAG |
| | | | CGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTGACCGA |
| | | | GCAGGACAGCAAGGACAGCACCTACAGCCTGAGCAGCA |
| | | | CCCTGACCCTGAGCAAGGCCGACTACGAGAAGCACAAG |
| | | | GTGTACGCCTGCGAGGTGACCCACCAGGGCCTGAGCAG |
| | | | CCCCGTGACCAAGAGCTTCAACCGGGGCGAGTGC |
| Her2 Cysmab | 245 | Heavy | EVQLVESGGGLVQPGGSLRLSCAASGFNIKDTYIHWVR |
| WT HC | | Chain (Wild | QAPGKGLEWVARIYPTNGYTRYADSVKGRFTISADTSK |
| | | Type Fc) | NTAYLQMNSLRAEDTAVYYCSRWGGDGFYAMDYWGQGT |
| | | | LVTVSSASTKGPSVFPLAPSSKSTSGGTAALGCLVKDY |
| | | | FPCPVTVSWNSGALTSGVHTFPAVLQSSGLYSLSSVVT |
| | | | VPSSSLGTQTYICNVNHKPSNTKVDKRVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPCDI |
| | | | AVEWESNGQPENNYKTTPPVLDSDGSFFLYSKLTVDKS |
| | | | RWQQGNVFSCSVMHEALHNHYTQKSLSLSPGK |
| | 246 | DNA Heavy | GAGGTGCAGCTGGTGGAGAGCGGCGGCGGCCTGGTGCA |
| | | Chain | GCCCGGCGGCAGCCTGCGGCTGAGCTGCGCCGCCAGCG |
| | | | GCTTCAACATCAAGGACACCTACATCCACTGGGTGCGG |
| | | | CAGGCCCCCGGCAAGGGCCTGGAGTGGGTGGCCCGGAT |
| | | | CTACCCCACCAACGGCTACACCCGGTACGCCGACAGCG |
| | | | TGAAGGGCCGGTTCACCATCAGCGCCGACACCAGCAAG |
| | | | AACACCGCCTACCTGCAGATGAACAGCCTGCGGGCCGA |
| | | | GGACACCGCCGTGTACTACTGCAGCCGGTGGGGCGGCG |
| | | | ACGGCTTCTACGCCATGGACTACTGGGGCCAGGGCACC |
| | | | CTGGTGACCGTGAGCAGCGCCAGCACCAAGGGCCCCAG |
| | | | CGTGTTCCCCCTGGCCCCCAGCAGCAAGAGCACCAGCG |
| | | | GCGGCACCGCCGCCCTGGGCTGCCTGGTGAAGGACTAC |
| | | | TTCCCCTGCCCCGTGACCGTGAGCTGGAACAGCGGCGC |
| | | | CCTGACCAGCGGCGTGCACACCTTCCCCGCCGTGCTGC |
| | | | AGAGCAGCGGCCTGTACAGCCTGAGCAGCGTGGTGACC |
| | | | GTGCCCAGCAGCAGCCTGGGCACCCAGACCTACATCTG |
| | | | CAACGTGAACCACAAGCCCAGCAACACCAAGGTGGACA |
| | | | AGCGGGTGGAGCCCAAGAGCTGCGACAAGACCCACACC |
| | | | TGCCCCCCCTGCCCCGCCCCCGAGCTGCTGGGCGGCCC |
| | | | CAGCGTGTTCCTGTTCCCCCCCAAGCCCAAGGACACCC |
| | | | TGATGATCAGCCGGACCCCCGAGGTGACCTGCGTGGTG |
| | | | GTGGACGTGAGCCACGAGGACCCCGAGGTGAAGTTCAA |
| | | | CTGGTACGTGGACGGCGTGGAGGTGCACAACGCCAAGA |
| | | | CCAAGCCCCGGGAGGAGCAGTACAACAGCACCTACCGG |
| | | | GTGGTGAGCGTGCTGACCGTGCTGCACCAGGACTGGCT |
| | | | GAACGGCAAGGAGTACAAGTGCAAGGTGAGCAACAAGG |
| | | | CCCTGCCCGCCCCCATCGAGAAGACCATCAGCAAGGCC |
| | | | AAGGGCCAGCCCCGGGAGCCCCAGGTGTACACCCTGCC |
| | | | CCCCAGCCGGGAGGAGATGACCAAGAACCAGGTGAGCC |
| | | | TGACCTGCCTGGTGAAGGGCTTCTACCCCTGCGACATC |
| | | | GCCGTGGAGTGGGAGAGCAACGGCCAGCCCGAGAACAA |
| | | | CTACAAGACCACCCCCCCCGTGCTGGACAGCGACGGCA |
| | | | GCTTCTTCCTGTACAGCAAGCTGACCGTGGACAAGAGC |
| | | | CGGTGGCAGCAGGGCAACGTGTTCAGCTGCAGCGTGAT |
| | | | GCACGAGGCCCTGCACAACCACTACACCCAGAAGAGCC |
| | | | TGAGCCTGAGCCCCGGCAAG |
| | 66 | Light Chain | DIQMTQSPSSLSASVGDRVTITCRASQDVNTAVAWYQQ |
| | | | KPGKAPKLLIYSASFLYSGVPSRFSGSRSGTDFTLTIS |
| | | | SLQPEDFATYYCQQHYTTPPTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 247 | DNA Light | GACATCCAGATGACCCAGAGCCCCAGCAGCCTGAGCGC |
| | | Chain | CAGCGTGGGCGACCGGGTGACCATCACCTGCCGGGCCA |
| | | | GCCAGGACGTGAACACCGCCGTGGCCTGGTACCAGCAG |
| | | | AAGCCCGGCAAGGCCCCCAAGCTGCTGATCTACAGCGC |
| | | | CAGCTTCCTGTACAGCGGCGTGCCCAGCCGGTTCAGCG |
| | | | GCAGCCGGAGCGGCACCGACTTCACCCTGACCATCAGC |
| | | | AGCCTGCAGCCCGAGGACTTCGCCACCTACTACTGCCA |
| | | | GCAGCACTACACCACCCCCCCCACCTTCGGCCAGGGCA |
| | | | CCAAGGTGGAGATCAAGCGGACCGTGGCCGCCCCCAGC |
| | | | GTGTTCATCTTCCCCCCCAGCGACGAGCAGCTGAAGAG |
| | | | CGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTGACCGA |
| | | | GCAGGACAGCAAGGACAGCACCTACAGCCTGAGCAGCA |
| | | | CCCTGACCCTGAGCAAGGCCGACTACGAGAAGCACAAG |
| | | | GTGTACGCCTGCGAGGTGACCCACCAGGGCCTGAGCAG |
| | | | CCCCGTGACCAAGAGCTTCAACCGGGGCGAGTGC |
| PCAD | 248 | Heavy | EVQLVQSGAEVKKPGESLKISCKVSGYTFTDHTIHWMR |
| CQY679 | | Chain (Wild | QMPGKGLEWMGYIYPRSGSINYNEKFKGQVTISADKSS |
| Cysmab WT | | Type Fc) | STAYLQWSSLKASDTAMYYCARRNLFLPMEYWGQGTLV |
| HC | | | TVSSASTKGPSVFPLAPSSKSTSGGTAALGCLVKDYFP |
| | | | EPVTVSWNSGALTSGVHTFPAVLQSSGLYSLSSVVTVP |
| | | | SSSLGTQTYICNVNHKPSNTKVDKRVEPKSCDKTHTCP |
| | | | PCPAPELLGGPSVFLFPPKPKDTLMISRTPEVTCVVVD |
| | | | VSHEDPEVKFNWYVDGVEVHNAKTKPREEQYNSTYRVV |
| | | | SVLTVLHQDWLNGKEYKCKVSNKALPAPIEKTISKAKG |
| | | | QPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| | 249 | DNA Heavy | GAGGTGCAGCTGGTGCAGAGCGGCGCCGAGGTGAAGAA |
| | | Chain | GCCCGGCGAGAGCCTGAAGATCAGCTGCAAGGTGAGCG |
| | | | GCTACACCTTCACCGACCACACCATCCACTGGATGCGG |
| | | | CAGATGCCCGGCAAGGGCCTGGAGTGGATGGGCTACAT |
| | | | CTACCCCCGGAGCGGCAGCATCAACTACAACGAGAAGT |
| | | | TCAAGGGCCAGGTGACCATCAGCGCCGACAAGAGCAGC |
| | | | AGCACCGCCTACCTGCAGTGGAGCAGCCTGAAGGCCAG |
| | | | CGACACCGCCATGTACTACTGCGCCCGGCGGAACCTGT |
| | | | TCCTGCCCATGGAGTACTGGGGCCAGGGCACCCTGGTG |
| | | | ACCGTGAGCAGCGCCAGCACCAAGGGCCCCAGCGTGTT |
| | | | CCCCCTGGCCCCCAGCAGCAAGAGCACCAGCGGCGGCA |
| | | | CCGCCGCCCTGGGCTGCCTGGTGAAGGACTACTTCCCC |
| | | | GAGCCCGTGACCGTGAGCTGGAACAGCGGCGCCCTGAC |
| | | | CAGCGGCGTGCACACCTTCCCCGCCGTGCTGCAGAGCA |
| | | | GCGGCCTGTACAGCCTGAGCAGCGTGGTGACCGTGCCC |
| | | | AGCAGCAGCCTGGGCACCCAGACCTACATCTGCAACGT |
| | | | GAACCACAAGCCCAGCAACACCAAGGTGGACAAGCGGG |
| | | | TGGAGCCCAAGAGCTGCGACAAGACCCACACCTGCCCC |
| | | | CCCTGCCCCGCCCCCGAGCTGCTGGGCGGCCCCAGCGT |
| | | | GTTCCTGTTCCCCCCCAAGCCCAAGGACACCCTGATGA |
| | | | TCAGCCGGACCCCCGAGGTGACCTGCGTGGTGGTGGAC |
| | | | GTGAGCCACGAGGACCCCGAGGTGAAGTTCAACTGGTA |
| | | | CGTGGACGGCGTGGAGGTGCACAACGCCAAGACCAAGC |
| | | | CCCGGGAGGAGCAGTACAACAGCACCTACCGGGTGGTG |
| | | | AGCGTGCTGACCGTGCTGCACCAGGACTGGCTGAACGG |
| | | | CAAGGAGTACAAGTGCAAGGTGAGCAACAAGGCCCTGC |
| | | | CCGCCCCCATCGAGAAGACCATCAGCAAGGCCAAGGGC |
| | | | CAGCCCCGGGAGCCCCAGGTGTACACCCTGCCCCCCAG |
| | | | CCGGGAGGAGATGACCAAGAACCAGGTGAGCCTGACCT |
| | | | GCCTGGTGAAGGGCTTCTACCCCAGCGACATCGCCGTG |
| | | | GAGTGGGAGAGCAACGGCCAGCCCGAGAACAACTACAA |
| | | | GACCACCCCCCCCGTGCTGGACAGCGACGGCAGCTTCT |
| | | | TCCTGTACAGCAAGCTGACCGTGGACAAGAGCCGGTGG |
| | | | CAGCAGGGCAACGTGTTCAGCTGCAGCGTGATGCACGA |
| | | | GGCCCTGCACAACCACTACACCCAGAAGAGCCTGAGCC |
| | | | TGAGCCCCGGCAAG |
| | 250 | Light Chain | DIVMTQTPLSLPVTPGEPASISCRSSQSLLSSGDQKNY |
| | | | LTWYLQKPGQSPQLLIYWASTRESGVPDRFSGSGSGTD |
| | | | FTLKISRVEAEDVGVYYCQNDYRYPLTFGQGTKLEIKR |
| | | | TVAAPSVFIFPPSDEQLKSGTASVVCLLNNFYPREAKV |
| | | | QWKVDNALQSGNSQESVTEQDSKDSTYSLSSTLTLSKA |
| | | | DYEKHKVYACEVTHQGLSSPVIKSENRGEC |
| | 251 | DNA Light | GACATCGTGATGACCCAGACCCCCCTGAGCCTGCCCGT |
| | | Chain | GACCCCCGGCGAGCCCGCCAGCATCAGCTGCCGGAGCA |
| | | | GCCAGAGCCTGCTGAGCAGCGGCGACCAGAAGAACTAC |
| | | | CTGACCTGGTACCTGCAGAAGCCCGGCCAGAGCCCCCA |
| | | | GCTGCTGATCTACTGGGCCAGCACCCGGGAGAGCGGCG |
| | | | TGCCCGACCGGTTCAGCGGCAGCGGCAGCGGCACCGAC |
| | | | TTCACCCTGAAGATCAGCCGGGTGGAGGCCGAGGACGT |
| | | | GGGCGTGTACTACTGCCAGAACGACTACCGGTACCCCC |
| | | | TGACCTTCGGCCAGGGCACCAAGCTGGAGATCAAGCGG |
| | | | ACCGTGGCCGCCCCCAGCGTGTTCATCTTCCCCCCCAG |
| | | | CGACGAGCAGCTGAAGAGCGGCACCGCCAGCGTGGTGT |
| | | | GCCTGCTGAACAACTTCTACCCCCGGGAGGCCAAGGTG |
| | | | CAGTGGAAGGTGGACAACGCCCTGCAGAGCGGCAACAG |
| | | | CCAGGAGAGCGTGACCGAGCAGGACAGCAAGGACAGCA |
| | | | CCTACAGCCTGAGCAGCACCCTGACCCTGAGCAAGGCC |
| | | | GACTACGAGAAGCACAAGGTGTACGCCTGCGAGGTGAC |
| | | | CCACCAGGGCCTGAGCAGCCCCGTGACCAAGAGCTTCA |
| | | | ACCGGGGCGAGTGC |
| EphA2 1C1 | 252 | Heavy | EVQLLESGGGLVQPGGSLRLSCAASGFTFSHYMMAWVR |
| Cysmab WT | | Chain (Wild | QAPGKGLEWVSRIGPSGGPTHYADSVKGRFTISRDNSK |
| HC | | Type Fc) | NTLYLQMNSLRAEDTAVYYCAGYDSGYDYVAVAGPAEY |
| | | | FQHWGQGTLVTVSSASTKGPSVFPLAPSSKSTSGGTAA |
| | | | LGCLVKDYFPCPVTVSWNSGALTSGVHTFPAVLQSSGL |
| | | | YSLSSVVTVPSSSLGTQTYICNVNHKPSNTKVDKRVEP |
| | | | KSCDKTHTCPPCPAPELLGGPSVFLFPPKPKDTLMISR |
| | | | TPEVTCVVVDVSHEDPEVKFNWYVDGVEVHNAKTKPRE |
| | | | EQYNSTYRVVSVLTVLHQDWLNGKEYKCKVSNKALPAP |
| | | | IEKTISKAKGQPREPQVYTLPPSREEMTKNQVSLTCLV |
| | | | KGFYPCDIAVEWESNGQPENNYKTTPPVLDSDGSFFLY |
| | | | SKLTVDKSRWQQGNVFSCSVMHEALHNHYTQKSLSLSP |
| | | | GK |
| | 253 | DNA Heavy | GAAGTTCAGCTGCTTGAATCTGGCGGCGGACTGGTTCA |
| | | Chain | ACCTGGCGGATCTCTGAGACTGAGCTGTGCCGCCAGCG |
| | | | GCTTCACCTTCAGCCACTATATGATGGCCTGGGTCCGA |
| | | | CAGGCCCCTGGCAAAGGACTTGAATGGGTGTCCAGAAT |
| | | | CGGCCCCTCTGGCGGCCCTACACACTACGCTGATTCTG |
| | | | TGAAGGGCAGATTCACCATCAGCCGGGACAACAGCAAG |
| | | | AACACCCTGTACCTGCAGATGAACAGCCTGAGAGCCGA |
| | | | GGACACCGCCGTGTATTACTGTGCCGGCTACGACAGCG |
| | | | GCTACGATTATGTGGCTGTGGCCGGACCTGCCGAGTAC |
| | | | TTTCAGCATTGGGGACAGGGCACCCTGGTCACCGTTAG |
| | | | TTCTGCTAGCACCAAGGGCCCAAGTGTGTTTCCCCTGG |
| | | | CCCCCAGCAGCAAGTCTACTTCCGGCGGAACTGCTGCC |
| | | | CTGGGTTGCCTGGTGAAGGACTACTTCCCCTGTCCCGT |
| | | | GACAGTGTCCTGGAACTCTGGGGCTCTGACTTCCGGCG |
| | | | TGCACACCTTCCCCGCCGTGCTGCAGAGCAGCGGCCTG |
| | | | TACAGCCTGAGCAGCGTGGTGACAGTGCCCTCCAGCTC |
| | | | TCTGGGAACCCAGACCTATATCTGCAACGTGAACCACA |
| | | | AGCCCAGCAACACCAAGGTGGACAAGAGAGTGGAGCCC |
| | | | AAGAGCTGCGACAAGACCCACACCTGCCCCCCCTGCCC |
| | | | AGCTCCAGAACTGCTGGGAGGGCCTTCCGTGTTCCTGT |
| | | | TCCCCCCCAAGCCCAAGGACACCCTGATGATCAGCAGG |
| | | | ACCCCCGAGGTGACCTGCGTGGTGGTGGACGTGTCCCA |
| | | | CGAGGACCCAGAGGTGAAGTTCAACTGGTACGTGGACG |
| | | | GCGTGGAGGTGCACAACGCCAAGACCAAGCCCAGAGAG |
| | | | GAGCAGTACAACAGCACCTACAGGGTGGTGTCCGTGCT |
| | | | GACCGTGCTGCACCAGGACTGGCTGAACGGCAAAGAAT |
| | | | ACAAGTGCAAAGTCTCCAACAAGGCCCTGCCAGCCCCA |
| | | | ATCGAAAAGACAATCAGCAAGGCCAAGGGCCAGCCACG |
| | | | GGAGCCCCAGGTGTACACCCTGCCCCCCAGCCGGGAGG |
| | | | AGATGACCAAGAACCAGGTGTCCCTGACCTGTCTGGTG |
| | | | AAGGGCTTCTACCCCTGTGATATCGCCGTGGAGTGGGA |
| | | | GAGCAACGGCCAGCCCGAGAACAACTACAAGACCACCC |
| | | | CCCCAGTGCTGGACAGCGACGGCAGCTTCTTCCTGTAC |
| | | | AGCAAGCTGACCGTGGACAAGTCCAGGTGGCAGCAGGG |
| | | | CAACGTGTTCAGCTGCAGCGTGATGCACGAGGCCCTGC |
| | | | ACAACCACTACACCCAGAAGTCCCTGAGCCTGAGCCCC |
| | | | GGCAAG |
| | 254 | Light Chain | DIQMTQSPSSLSASVGDRVTITCRASQSISTWLAWYQQ |
| | | | KPGKAPKLLIYKASNLHTGVPSRFSGSGSGTEFSLTIS |
| | | | GLQPDDFATYYCQQYNSYSRTFGQGTKVEIKRTVAAPS |
| | | | VFIFPPSDEQLKSGTASVVCLLNNFYPREAKVQWKVDN |
| | | | ALQSGNSQESVTEQDSKDSTYSLSSTLTLSKADYEKHK |
| | | | VYACEVTHQGLSSPVTKSFNRGEC |
| | 255 | DNA Light | GACATTCAGATGACACAGAGCCCTAGCAGCCTGAGCGC |
| | | Chain | CTCTGTGGGAGACAGAGTGACCATCACCTGTAGAGCCA |
| | | | GCCAGAGCATCAGCACATGGCTGGCCTGGTATCAGCAG |
| | | | AAGCCTGGCAAGGCCCCTAAGCTGCTGATCTACAAGGC |
| | | | CAGCAATCTGCACACCGGCGTGCCCAGCAGATTTTCTG |
| | | | GCTCTGGAAGCGGCACCGAGTTCAGCCTGACAATCTCT |
| | | | GGCCTGCAGCCTGACGACTTCGCCACCTACTACTGCCA |
| | | | GCAGTACAACAGCTACAGCCGGACCTTTGGCCAGGGAA |
| | | | CAAAGGTGGAAATCAAGCGTACGGTGGCCGCTCCCAGC |
| | | | GTGTTCATCTTCCCCCCCAGCGACGAGCAGCTGAAGAG |
| | | | TGGCACCGCCAGCGTGGTGTGCCTGCTGAACAACTTCT |
| | | | ACCCCCGGGAGGCCAAGGTGCAGTGGAAGGTGGACAAC |
| | | | GCCCTGCAGAGCGGCAACAGCCAGGAGAGCGTCACCGA |
| | | | GCAGGACAGCAAGGACTCCACCTACAGCCTGAGCAGCA |
| | | | CCCTGACCCTGAGCAAGGCCGACTACGAGAAGCATAAG |
| | | | GTGTACGCCTGCGAGGTGACCCACCAGGGCCTGTCCAG |
| | | | CCCCGTGACCAAGAGCTTCAACAGGGGCGAGTGC |
| HER2 | 342 | Heavy | EVQLVQSGAEVKKPGATVKISCKVSGYTFTDYYIH |
| Disitamab | | Chain | WVQQAPGKGLEWMGRVNPDHGDSYYNQKFKDK |
| | | (E152C | ATITADKSTDTAYMELSSLRSEDTAVYFCARNYLF |
| | | S375C) | DHWGQGTLVTVSSASTKGPSVFPLAPSSKSTSG |
| | | | GTAALGCLVKDYFPCPVTVSWNSGALTSGVHTFP |
| | | | AVLQSSGLYSLSSVVTVPSSSLGTQTYICNVNHKP |
| | | | SNTKVDKRVEPKSCDKTHTCPPCPAPELLGGPSV |
| | | | FLFPPKPKDTLMISRTPEVTCVVVDVSHEDPEVKF |
| | | | NWYVDGVEVHNAKTKPREEQYNSTYRVVSVLTV |
| | | | LHQDWLNGKEYKCKVSNKALPAPIEKTISKAKGQ |
| | | | PREPQVYTLPPSREEMTKNQVSLTCLVKGFYPCD |
| | | | IAVEWESNGQPENNYKTTPPVLDSDGSFFLYSKL |
| | | | TVDKSRWQQGNVFSCSVMHEALHNHYTQKSLSL |
| | | | SPGK |
| | 343 | DNA | GAAGTTCAGCTGGTTCAGTCTGGCGCCGAAGT |
| | | Heavy | GAAGAAACCTGGCGCCACCGTGAAGATCAGCT |
| | | Chain | GCAAGGTGTCCGGCTACACCTTCACCGACTACT |
| | | (E152C | ACATCCACTGGGTGCAGCAGGCCCCTGGCAAA |
| | | S375C) | GGACTTGAGTGGATGGGCAGAGTGAACCCCGA |
| | | | TCACGGCGACAGCTACTACAACCAGAAGTTCAA |
| | | | GGACAAGGCCACCATCACCGCCGACAAGAGCA |
| | | | CCGATACCGCCTACATGGAACTGAGCAGCCTG |
| | | | AGAAGCGAGGATACCGCCGTGTACTTCTGCGC |
| | | | CCGGAACTACCTGTTTGACCACTGGGGACAGG |
| | | | GCACCCTGGTCACAGTTAGTTCTGCTAGCACCA |
| | | | AGGGCCCAAGTGTGTTTCCCCTGGCCCCCAGC |
| | | | AGCAAGTCTACTTCCGGCGGAACTGCTGCCCT |
| | | | GGGTTGCCTGGTGAAGGACTACTTCCCCTGTC |
| | | | CCGTGACAGTGTCCTGGAACTCTGGGGCTCTG |
| | | | ACTTCCGGCGTGCACACCTTCCCCGCCGTGCT |
| | | | GCAGAGCAGCGGCCTGTACAGCCTGAGCAGCG |
| | | | TGGTGACAGTGCCCTCCAGCTCTCTGGGAACC |
| | | | CAGACCTATATCTGCAACGTGAACCACAAGCCC |
| | | | AGCAACACCAAGGTGGACAAGAGAGTGGAGCC |
| | | | CAAGAGCTGCGACAAGACCCACACCTGCCCCC |
| | | | CCTGCCCAGCTCCAGAACTGCTGGGAGGGCCT |
| | | | TCCGTGTTCCTGTTCCCCCCCAAGCCCAAGGAC |
| | | | ACCCTGATGATCAGCAGGACCCCCGAGGTGAC |
| | | | CTGCGTGGTGGTGGACGTGTCCCACGAGGACC |
| | | | CAGAGGTGAAGTTCAACTGGTACGTGGACGGC |
| | | | GTGGAGGTGCACAACGCCAAGACCAAGCCCAG |
| | | | AGAGGAGCAGTACAACAGCACCTACAGGGTGG |
| | | | TGTCCGTGCTGACCGTGCTGCACCAGGACTGG |
| | | | CTGAACGGCAAAGAATACAAGTGCAAAGTCTCC |
| | | | AACAAGGCCCTGCCAGCCCCAATCGAAAAGAC |
| | | | AATCAGCAAGGCCAAGGGCCAGCCACGGGAGC |
| | | | CCCAGGTGTACACCCTGCCCCCCAGCCGGGAG |
| | | | GAGATGACCAAGAACCAGGTGTCCCTGACCTG |
| | | | TCTGGTGAAGGGCTTCTACCCCTGTGATATCGC |
| | | | CGTGGAGTGGGAGAGCAACGGCCAGCCCGAG |
| | | | AACAACTACAAGACCACCCCCCCAGTGCTGGA |
| | | | CAGCGACGGCAGCTTCTTCCTGTACAGCAAGCT |
| | | | GACCGTGGACAAGTCCAGGTGGCAGCAGGGCA |
| | | | ACGTGTTCAGCTGCAGCGTGATGCACGAGGCC |
| | | | CTGCACAACCACTACACCCAGAAGTCCCTGAGC |
| | | | CTGAGCCCCGGCAAG |
| | 344 | Light Chain | DIQMTQSPSSVSASVGDRVTITCKASQDVGTAVA |
| | | | WYQQKPGKAPKLLIYWASIRHTGVPSRFSGSGSG |
| | | | TDFTLTISSLQPEDFATYYCHQFATYTFGGGTKVE |
| | | | IKRTVAAPSVFIFPPSDEQLKSGTASVVCLLNNFYP |
| | | | REAKVQWKVDNALQSGNSQESVTEQDSKDSTYS |
| | | | LSSTLTLSKADYEKHKVYACEVTHQGLSSPVTKSF |
| | | | NRGEC |
| | 345 | DNA Light | GACATTCAGATGACACAGAGCCCTAGCAGCGT |
| | | Chain | GTCCGCCTCTGTGGGAGACAGAGTGACCATCA |
| | | | CATGCAAGGCCAGCCAGGATGTGGGAACAGCC |
| | | | GTGGCTTGGTATCAGCAGAAGCCTGGCAAGGC |
| | | | CCCTAAGCTGCTGATCTACTGGGCCAGCATCAG |
| | | | ACACACAGGCGTGCCCAGCAGATTTTCTGGCA |
| | | | GCGGCTCTGGCACCGACTTCACCCTGACCATAT |
| | | | CTAGCCTGCAGCCAGAGGACTTCGCCACCTAC |
| | | | TACTGCCACCAGTTTGCCACCTACACCTTCGGC |
| | | | GGAGGCACCAAGGTGGAAATCAAGCGTACGGT |
| | | | GGCCGCTCCCAGCGTGTTCATCTTCCCCCCCA |
| | | | GCGACGAGCAGCTGAAGAGTGGCACCGCCAGC |
| | | | GTGGTGTGCCTGCTGAACAACTTCTACCCCCG |
| | | | GGAGGCCAAGGTGCAGTGGAAGGTGGACAACG |
| | | | CCCTGCAGAGCGGCAACAGCCAGGAGAGCGTC |
| | | | ACCGAGCAGGACAGCAAGGACTCCACCTACAG |
| | | | CCTGAGCAGCACCCTGACCCTGAGCAAGGCCG |
| | | | ACTACGAGAAGCATAAGGTGTACGCCTGCGAG |
| | | | GTGACCCACCAGGGCCTGTCCAGCCCCGTGAC |
| | | | CAAGAGCTTCAACAGGGGCGAGTGC |
| 5T4 | 346 | Heavy | EVQLVESGGGLVQPGGSLRLSCAASGYTFTNFG |
| Cysmab | | Chain | MNWVRQAPGKGLEWVAWINTNTGEPRYAEEFK |
| | | (Wild Type | GRFTISRDNAKNSLYLQMNSLRAEDTAVYYCARD |
| | | Fc) | WDGAYFFDYWGQGTLVTVSSASTKGPSVFPLAP |
| | | | SSKSTSGGTAALGCLVKDYFPCPVTVSWNSGALT |
| | | | SGVHTFPAVLQSSGLYSLSSVVTVPSSSLGTQTYI |
| | | | CNVNHKPSNTKVDKRVEPKSCDKTHTCPPCPAP |
| | | | ELLGGPSVFLFPPKPKDTLMISRTPEVTCVVVDVS |
| | | | HEDPEVKFNWYVDGVEVHNAKTKPREEQYNSTY |
| | | | RVVSVLTVLHQDWLNGKEYKCKVSNKALPAPIEK |
| | | | TISKAKGQPREPQVYTLPPSREEMTKNQVSLTCL |
| | | | VKGFYPCDIAVEWESNGQPENNYKTTPPVLDSDG |
| | | | SFFLYSKLTVDKSRWQQGNVFSCSVMHEALHNH |
| | | | YTQKSLSLSPGK |
| | 347 | DNA | GAAGTGCAGCTGGTTGAATCTGGCGGAGGACT |
| | | Heavy | GGTTCAGCCTGGCGGATCTCTGAGACTGTCTTG |
| | | Chain | TGCCGCCAGCGGCTACACCTTCACCAACTTCG |
| | | (Wild Type | GCATGAACTGGGTCCGACAGGCCCCTGGCAAA |
| | | Fc) | GGCCTTGAATGGGTCGCCTGGATCAACACCAAT |
| | | | ACCGGCGAGCCCAGATACGCCGAAGAGTTCAA |
| | | | GGGCAGATTCACCATCAGCCGGGACAACGCCA |
| | | | AGAACAGCCTGTACCTGCAGATGAACTCCCTGA |
| | | | GAGCCGAGGACACCGCCGTGTACTACTGTGCC |
| | | | AGAGATTGGGACGGCGCCTACTTCTTCGATTAC |
| | | | TGGGGCCAGGGCACCCTGGTCACAGTTAGTTC |
| | | | TGCTAGCACCAAGGGCCCAAGTGTGTTTCCCCT |
| | | | GGCCCCCAGCAGCAAGTCTACTTCCGGCGGAA |
| | | | CTGCTGCCCTGGGTTGCCTGGTGAAGGACTAC |
| | | | TTCCCCTGTCCCGTGACAGTGTCCTGGAACTCT |
| | | | GGGGCTCTGACTTCCGGCGTGCACACCTTCCC |
| | | | CGCCGTGCTGCAGAGCAGCGGCCTGTACAGCC |
| | | | TGAGCAGCGTGGTGACAGTGCCCTCCAGCTCT |
| | | | CTGGGAACCCAGACCTATATCTGCAACGTGAAC |
| | | | CACAAGCCCAGCAACACCAAGGTGGACAAGAG |
| | | | AGTGGAGCCCAAGAGCTGCGACAAGACCCACA |
| | | | CCTGCCCCCCCTGCCCAGCTCCAGAACTGCTG |
| | | | GGAGGGCCTTCCGTGTTCCTGTTCCCCCCCAA |
| | | | GCCCAAGGACACCCTGATGATCAGCAGGACCC |
| | | | CCGAGGTGACCTGCGTGGTGGTGGACGTGTCC |
| | | | CACGAGGACCCAGAGGTGAAGTTCAACTGGTA |
| | | | CGTGGACGGCGTGGAGGTGCACAACGCCAAGA |
| | | | CCAAGCCCAGAGAGGAGCAGTACAACAGCACC |
| | | | TACAGGGTGGTGTCCGTGCTGACCGTGCTGCA |
| | | | CCAGGACTGGCTGAACGGCAAAGAATACAAGT |
| | | | GCAAAGTCTCCAACAAGGCCCTGCCAGCCCCA |
| | | | ATCGAAAAGACAATCAGCAAGGCCAAGGGCCA |
| | | | GCCACGGGAGCCCCAGGTGTACACCCTGCCCC |
| | | | CCAGCCGGGAGGAGATGACCAAGAACCAGGTG |
| | | | TCCCTGACCTGTCTGGTGAAGGGCTTCTACCCC |
| | | | TGTGATATCGCCGTGGAGTGGGAGAGCAACGG |
| | | | CCAGCCCGAGAACAACTACAAGACCACCCCCC |
| | | | CAGTGCTGGACAGCGACGGCAGCTTCTTCCTG |
| | | | TACAGCAAGCTGACCGTGGACAAGTCCAGGTG |
| | | | GCAGCAGGGCAACGTGTTCAGCTGCAGCGTGA |
| | | | TGCACGAGGCCCTGCACAACCACTACACCCAG |
| | | | AAGTCCCTGAGCCTGAGCCCCGGCAAG |
| 5T4 DANAPA | 348 | Heavy | EVQLVESGGGLVQPGGSLRLSCAASGYTFTNFG |
| Cysmab | | Chain (Fc | MNWVRQAPGKGLEWVAWINTNTGEPRYAEEFK |
| | | Silenced | GRFTISRDNAKNSLYLQMNSLRAEDTAVYYCARD |
| | | DANAPA) | WDGAYFFDYWGQGTLVTVSSASTKGPSVFPLAP |
| | | | SSKSTSGGTAALGCLVKDYFPCPVTVSWNSGALT |
| | | | SGVHTFPAVLQSSGLYSLSSVVTVPSSSLGTQTYI |
| | | | CNVNHKPSNTKVDKRVEPKSCDKTHTCPPCPAP |
| | | | ELLGGPSVFLFPPKPKDTLMISRTPEVTCVVVAVS |
| | | | HEDPEVKFNWYVDGVEVHNAKTKPREEQYASTY |
| | | | RVVSVLTVLHQDWLNGKEYKCKVSNKALAAPIEK |
| | | | TISKAKGQPREPQVYTLPPSREEMTKNQVSLTCL |
| | | | VKGFYPCDIAVEWESNGQPENNYKTTPPVLDSDG |
| | | | SFFLYSKLTVDKSRWQQGNVFSCSVMHEALHNH |
| | | | YTQKSLSLSPGK |
| | 349 | DNA | GAAGTGCAGCTGGTTGAATCTGGCGGAGGACT |
| | | Heavy | GGTTCAGCCTGGCGGATCTCTGAGACTGTCTTG |
| | | Chain (Fc | TGCCGCCAGCGGCTACACCTTCACCAACTTCG |
| | | Silenced | GCATGAACTGGGTCCGACAGGCCCCTGGCAAA |
| | | DANAPA) | GGCCTTGAATGGGTCGCCTGGATCAACACCAAT |
| | | | ACCGGCGAGCCCAGATACGCCGAAGAGTTCAA |
| | | | GGGCAGATTCACCATCAGCCGGGACAACGCCA |
| | | | AGAACAGCCTGTACCTGCAGATGAACTCCCTGA |
| | | | GAGCCGAGGACACCGCCGTGTACTACTGTGCC |
| | | | AGAGATTGGGACGGCGCCTACTTCTTCGATTAC |
| | | | TGGGGCCAGGGCACCCTGGTCACAGTTAGTTC |
| | | | TGCTAGCACCAAGGGCCCAAGTGTGTTTCCCCT |
| | | | GGCCCCCAGCAGCAAGTCTACTTCCGGCGGAA |
| | | | CTGCTGCCCTGGGTTGCCTGGTGAAGGACTAC |
| | | | TTCCCCTGTCCCGTGACAGTGTCCTGGAACTCT |
| | | | GGGGCTCTGACTTCCGGCGTGCACACCTTCCC |
| | | | CGCCGTGCTGCAGAGCAGCGGCCTGTACAGCC |
| | | | TGAGCAGCGTGGTGACAGTGCCCTCCAGCTCT |
| | | | CTGGGAACCCAGACCTATATCTGCAACGTGAAC |
| | | | CACAAGCCCAGCAACACCAAGGTGGACAAGAG |
| | | | AGTGGAGCCCAAGAGCTGCGACAAGACCCACA |
| | | | CCTGCCCCCCCTGCCCAGCTCCAGAACTGCTG |
| | | | GGAGGGCCTTCCGTGTTCCTGTTCCCCCCCAA |
| | | | GCCCAAGGACACCCTGATGATCAGCAGGACCC |
| | | | CCGAGGTGACCTGCGTGGTGGTGGCCGTGTCC |
| | | | CACGAGGACCCAGAGGTGAAGTTCAACTGGTA |
| | | | CGTGGACGGCGTGGAGGTGCACAACGCCAAGA |
| | | | CCAAGCCCAGAGAGGAGCAGTACGCCAGCACC |
| | | | TACAGGGTGGTGTCCGTGCTGACCGTGCTGCA |
| | | | CCAGGACTGGCTGAACGGCAAAGAATACAAGT |
| | | | GCAAAGTCTCCAACAAGGCCCTGGCTGCCCCA |
| | | | ATCGAAAAGACAATCAGCAAGGCCAAGGGCCA |
| | | | GCCACGGGAGCCCCAGGTGTACACCCTGCCCC |
| | | | CCAGCCGGGAGGAGATGACCAAGAACCAGGTG |
| | | | TCCCTGACCTGTCTGGTGAAGGGCTTCTACCCC |
| | | | TGTGATATCGCCGTGGAGTGGGAGAGCAACGG |
| | | | CCAGCCCGAGAACAACTACAAGACCACCCCCC |
| | | | CAGTGCTGGACAGCGACGGCAGCTTCTTCCTG |
| | | | TACAGCAAGCTGACCGTGGACAAGTCCAGGTG |
| | | | GCAGCAGGGCAACGTGTTCAGCTGCAGCGTGA |
| | | | TGCACGAGGCCCTGCACAACCACTACACCCAG |
| | | | AAGTCCCTGAGCCTGAGCCCCGGCAAG |
| 5T4 LC | 350 | Light Chain | DIQMTQSPSSLSASVGDRVTITCKASQSVSNDVA |
| | | | WYQQKPGKAPKLLIYFATNRYTGVPSRFSGSGYG |
| | | | TDFTLTISSLQPEDFATYYCQQDYSSPWTFGQGT |
| | | | KVEIKRTVAAPSVFIFPPSDEQLKSGTASVVCLLN |
| | | | NFYPREAKVQWKVDNALQSGNSQESVTEQDSKD |
| | | | STYSLSSTLTLSKADYEKHKVYACEVTHQGLSSPV |
| | | | TKSFNRGEC |
| | 351 | DNA Light | GACATTCAGATGACACAGAGCCCTAGCAGCCT |
| | | Chain | GAGCGCCTCTGTGGGAGACAGAGTGACCATCA |
| | | | CATGCAAGGCCAGCCAGAGCGTGTCCAACGAC |
| | | | GTGGCATGGTATCAGCAGAAGCCTGGCAAGGC |
| | | | CCCTAAGCTGCTGATCTACTTCGCCACCAACAG |
| | | | ATACACCGGCGTGCCCAGCAGATTTTCCGGCT |
| | | | CTGGCTACGGCACCGACTTCACCCTGACCATAT |
| | | | CTAGCCTGCAGCCAGAGGATTTCGCCACCTACT |
| | | | ACTGCCAGCAGGACTACAGCAGCCCTTGGACA |
| | | | TTTGGCCAGGGAACAAAGGTGGAAATCAAGCG |
| | | | TACGGTGGCCGCTCCCAGCGTGTTCATCTTCCC |
| | | | CCCCAGCGACGAGCAGCTGAAGAGTGGCACCG |
| | | | CCAGCGTGGTGTGCCTGCTGAACAACTTCTACC |
| | | | CCCGGGAGGCCAAGGTGCAGTGGAAGGTGGA |
| | | | CAACGCCCTGCAGAGCGGCAACAGCCAGGAGA |
| | | | GCGTCACCGAGCAGGACAGCAAGGACTCCACC |
| | | | TACAGCCTGAGCAGCACCCTGACCCTGAGCAA |
| | | | GGCCGACTACGAGAAGCATAAGGTGTACGCCT |
| | | | GCGAGGTGACCCACCAGGGCCTGTCCAGCCCC |
| | | | GTGACCAAGAGCTTCAACAGGGGCGAGTGC |
| Trop2 | 352 | Heavy | QVQLVQSGAEVKKPGASVKVSCKASGYTFTTAG |
| Datopotamab | | Chain | MQWVRQAPGQGLEWMGWINTHSGVPKYAEDFK |
| Cysmab | | (Wild Type | GRVTISADTSTSTAYLQLSSLKSEDTAVYYCARSG |
| | | Fc) | FGSSYWYFDVWGQGTLVTVSSASTKGPSVFPLA |
| | | | PSSKSTSGGTAALGCLVKDYFPCPVTVSWNSGAL |
| | | | TSGVHTFPAVLQSSGLYSLSSVVTVPSSSLGTQT |
| | | | YICNVNHKPSNTKVDKRVEPKSCDKTHTCPPCPA |
| | | | PELLGGPSVFLFPPKPKDTLMISRTPEVTCVVVDV |
| | | | SHEDPEVKFNWYVDGVEVHNAKTKPREEQYNST |
| | | | YRVVSVLTVLHQDWLNGKEYKCKVSNKALPAPIE |
| | | | KTISKAKGQPREPQVYTLPPSREEMTKNQVSLTC |
| | | | LVKGFYPCDIAVEWESNGQPENNYKTTPPVLDSD |
| | | | GSFFLYSKLTVDKSRWQQGNVFSCSVMHEALHN |
| | | | HYTQKSLSLSPGK |
| | 353 | DNA | CAGGTTCAGCTGGTTCAGTCTGGCGCCGAAGT |
| | | Heavy | GAAGAAACCTGGCGCCTCTGTGAAGGTGTCCT |
| | | Chain | GCAAGGCCAGCGGCTACACCTTTACAACAGCC |
| | | (Wild Type | GGCATGCAGTGGGTCCGACAGGCTCCTGGACA |
| | | Fc) | AGGCTTGGAATGGATGGGCTGGATCAACACCC |
| | | | ACAGCGGCGTGCCCAAATACGCCGAGGATTTC |
| | | | AAGGGCAGAGTGACCATCAGCGCCGACACCAG |
| | | | CACAAGCACAGCCTACCTGCAGCTGAGCAGCC |
| | | | TGAAGTCTGAGGACACCGCCGTGTACTACTGTG |
| | | | CCAGAAGCGGCTTTGGCAGCAGCTACTGGTAC |
| | | | TTCGATGTGTGGGGCCAGGGCACCCTGGTCAC |
| | | | AGTTAGTTCTGCTAGCACCAAGGGCCCAAGTGT |
| | | | GTTTCCCCTGGCCCCCAGCAGCAAGTCTACTTC |
| | | | CGGCGGAACTGCTGCCCTGGGTTGCCTGGTGA |
| | | | AGGACTACTTCCCCTGTCCCGTGACAGTGTCCT |
| | | | GGAACTCTGGGGCTCTGACTTCCGGCGTGCAC |
| | | | ACCTTCCCCGCCGTGCTGCAGAGCAGCGGCCT |
| | | | GTACAGCCTGAGCAGCGTGGTGACAGTGCCCT |
| | | | CCAGCTCTCTGGGAACCCAGACCTATATCTGCA |
| | | | ACGTGAACCACAAGCCCAGCAACACCAAGGTG |
| | | | GACAAGAGAGTGGAGCCCAAGAGCTGCGACAA |
| | | | GACCCACACCTGCCCCCCCTGCCCAGCTCCAG |
| | | | AACTGCTGGGAGGGCCTTCCGTGTTCCTGTTCC |
| | | | CCCCCAAGCCCAAGGACACCCTGATGATCAGC |
| | | | AGGACCCCCGAGGTGACCTGCGTGGTGGTGGA |
| | | | CGTGTCCCACGAGGACCCAGAGGTGAAGTTCA |
| | | | ACTGGTACGTGGACGGCGTGGAGGTGCACAAC |
| | | | GCCAAGACCAAGCCCAGAGAGGAGCAGTACAA |
| | | | CAGCACCTACAGGGTGGTGTCCGTGCTGACCG |
| | | | TGCTGCACCAGGACTGGCTGAACGGCAAAGAA |
| | | | TACAAGTGCAAAGTCTCCAACAAGGCCCTGCCA |
| | | | GCCCCAATCGAAAAGACAATCAGCAAGGCCAA |
| | | | GGGCCAGCCACGGGAGCCCCAGGTGTACACC |
| | | | CTGCCCCCCAGCCGGGAGGAGATGACCAAGAA |
| | | | CCAGGTGTCCCTGACCTGTCTGGTGAAGGGCT |
| | | | TCTACCCCTGTGATATCGCCGTGGAGTGGGAG |
| | | | AGCAACGGCCAGCCCGAGAACAACTACAAGAC |
| | | | CACCCCCCCAGTGCTGGACAGCGACGGCAGCT |
| | | | TCTTCCTGTACAGCAAGCTGACCGTGGACAAGT |
| | | | CCAGGTGGCAGCAGGGCAACGTGTTCAGCTGC |
| | | | AGCGTGATGCACGAGGCCCTGCACAACCACTA |
| | | | CACCCAGAAGTCCCTGAGCCTGAGCCCCGGCA |
| | | | AG |
| Trop2 | 354 | Heavy | QVQLVQSGAEVKKPGASVKVSCKASGYTFTTAG |
| Datopotamab | | Chain (Fc | MQWVRQAPGQGLEWMGWINTHSGVPKYAEDFK |
| DANAPA | | Silenced | GRVTISADTSTSTAYLQLSSLKSEDTAVYYCARSG |
| Cysmab | | DANAPA) | FGSSYWYFDVWGQGTLVTVSSASTKGPSVFPLA |
| | | | PSSKSTSGGTAALGCLVKDYFPCPVTVSWNSGAL |
| | | | TSGVHTFPAVLQSSGLYSLSSVVTVPSSSLGTQT |
| | | | YICNVNHKPSNTKVDKRVEPKSCDKTHTCPPCPA |
| | | | PELLGGPSVFLFPPKPKDTLMISRTPEVTCVVVAV |
| | | | SHEDPEVKFNWYVDGVEVHNAKTKPREEQYAST |
| | | | YRVVSVLTVLHQDWLNGKEYKCKVSNKALAAPIE |
| | | | KTISKAKGQPREPQVYTLPPSREEMTKNQVSLTC |
| | | | LVKGFYPCDIAVEWESNGQPENNYKTTPPVLDSD |
| | | | GSFFLYSKLTVDKSRWQQGNVFSCSVMHEALHN |
| | | | HYTQKSLSLSPGK |
| | 355 | DNA | CAGGTTCAGCTGGTTCAGTCTGGCGCCGAAGT |
| | | Heavy | GAAGAAACCTGGCGCCTCTGTGAAGGTGTCCT |
| | | Chain (Fc | GCAAGGCCAGCGGCTACACCTTTACAACAGCC |
| | | Silenced | GGCATGCAGTGGGTCCGACAGGCTCCTGGACA |
| | | DANAPA) | AGGCTTGGAATGGATGGGCTGGATCAACACCC |
| | | | ACAGCGGCGTGCCCAAATACGCCGAGGATTTC |
| | | | AAGGGCAGAGTGACCATCAGCGCCGACACCAG |
| | | | CACAAGCACAGCCTACCTGCAGCTGAGCAGCC |
| | | | TGAAGTCTGAGGACACCGCCGTGTACTACTGTG |
| | | | CCAGAAGCGGCTTTGGCAGCAGCTACTGGTAC |
| | | | TTCGATGTGTGGGGCCAGGGCACCCTGGTCAC |
| | | | AGTTAGTTCTGCTAGCACCAAGGGCCCAAGTGT |
| | | | GTTTCCCCTGGCCCCCAGCAGCAAGTCTACTTC |
| | | | CGGCGGAACTGCTGCCCTGGGTTGCCTGGTGA |
| | | | AGGACTACTTCCCCTGTCCCGTGACAGTGTCCT |
| | | | GGAACTCTGGGGCTCTGACTTCCGGCGTGCAC |
| | | | ACCTTCCCCGCCGTGCTGCAGAGCAGCGGCCT |
| | | | GTACAGCCTGAGCAGCGTGGTGACAGTGCCCT |
| | | | CCAGCTCTCTGGGAACCCAGACCTATATCTGCA |
| | | | ACGTGAACCACAAGCCCAGCAACACCAAGGTG |
| | | | GACAAGAGAGTGGAGCCCAAGAGCTGCGACAA |
| | | | GACCCACACCTGCCCCCCCTGCCCAGCTCCAG |
| | | | AACTGCTGGGAGGGCCTTCCGTGTTCCTGTTCC |
| | | | CCCCCAAGCCCAAGGACACCCTGATGATCAGC |
| | | | AGGACCCCCGAGGTGACCTGCGTGGTGGTGGC |
| | | | CGTGTCCCACGAGGACCCAGAGGTGAAGTTCA |
| | | | ACTGGTACGTGGACGGCGTGGAGGTGCACAAC |
| | | | GCCAAGACCAAGCCCAGAGAGGAGCAGTACGC |
| | | | CAGCACCTACAGGGTGGTGTCCGTGCTGACCG |
| | | | TGCTGCACCAGGACTGGCTGAACGGCAAAGAA |
| | | | TACAAGTGCAAAGTCTCCAACAAGGCCCTGGCT |
| | | | GCCCCAATCGAAAAGACAATCAGCAAGGCCAA |
| | | | GGGCCAGCCACGGGAGCCCCAGGTGTACACC |
| | | | CTGCCCCCCAGCCGGGAGGAGATGACCAAGAA |
| | | | CCAGGTGTCCCTGACCTGTCTGGTGAAGGGCT |
| | | | TCTACCCCTGTGATATCGCCGTGGAGTGGGAG |
| | | | AGCAACGGCCAGCCCGAGAACAACTACAAGAC |
| | | | CACCCCCCCAGTGCTGGACAGCGACGGCAGCT |
| | | | TCTTCCTGTACAGCAAGCTGACCGTGGACAAGT |
| | | | CCAGGTGGCAGCAGGGCAACGTGTTCAGCTGC |
| | | | AGCGTGATGCACGAGGCCCTGCACAACCACTA |
| | | | CACCCAGAAGTCCCTGAGCCTGAGCCCCGGCA |
| | | | AG |
| Trop2 | 356 | Light Chain | DIQMTQSPSSLSASVGDRVTITCKASQDVSTAVA |
| Datopotamab | | | WYQQKPGKAPKLLIYSASYRYTGVPSRFSGSGSG |
| LC | | | TDFTLTISSLQPEDFAVYYCQQHYITPLTFGQGTK |
| | | | LEIKRTVAAPSVFIFPPSDEQLKSGTASVVCLLNNF |
| | | | YPREAKVQWKVDNALQSGNSQESVTEQDSKDST |
| | | | YSLSSTLTLSKADYEKHKVYACEVTHQGLSSPVTK |
| | | | SFNRGEC |
| | 357 | DNA Light | GACATTCAGATGACACAGAGCCCTAGCAGCCT |
| | | Chain | GAGCGCCTCTGTGGGAGACAGAGTGACCATCA |
| | | | CATGCAAGGCCAGCCAGGATGTGTCTACAGCC |
| | | | GTGGCCTGGTATCAGCAGAAGCCTGGAAAGGC |
| | | | CCCTAAGCTGCTGATCTACAGCGCCAGCTACAG |
| | | | ATACACCGGCGTGCCCAGCAGATTTTCTGGCA |
| | | | GCGGCTCTGGCACCGACTTCACCCTGACCATAT |
| | | | CTAGCCTGCAGCCAGAGGACTTCGCCGTGTAC |
| | | | TACTGCCAGCAGCACTACATCACCCCTCTGACC |
| | | | TTTGGCCAGGGGACCAAGCTGGAAATCAAGCG |
| | | | TACGGTGGCCGCTCCCAGCGTGTTCATCTTCCC |
| | | | CCCCAGCGACGAGCAGCTGAAGAGTGGCACCG |
| | | | CCAGCGTGGTGTGCCTGCTGAACAACTTCTACC |
| | | | CCCGGGAGGCCAAGGTGCAGTGGAAGGTGGA |
| | | | CAACGCCCTGCAGAGCGGCAACAGCCAGGAGA |
| | | | GCGTCACCGAGCAGGACAGCAAGGACTCCACC |
| | | | TACAGCCTGAGCAGCACCCTGACCCTGAGCAA |
| | | | GGCCGACTACGAGAAGCATAAGGTGTACGCCT |
| | | | GCGAGGTGACCCACCAGGGCCTGTCCAGCCCC |
| | | | GTGACCAAGAGCTTCAACAGGGGCGAGTGCTG |
| | | | A |

TABLE D8
Exemplary Antibody CDR and Variable Region Sequences

| | SEQ | | |
|---|---|---|---|
| | ID | | |
| Ab | NO | IgG chain | Amino acid sequence |
| CD74 | 153 | VH (Wild | QVQLQQSGSELKKPGASVKVSCKASGYTFTNYGVNWI |
| milatuzumab | | Type Fc) | KQAPGQGLQWMGWINPNTGEPTFDDDFKGRFAFSLDT |
| Cysmab HC | | | SVSTAYLQISSLKADDTAVYFCSRSRGKNEAWFAYWG |
| (VHmil x | | | QGTLVTVSS |
| VKlaNQ) | | | |
| HCDR1 | 256 | Chothia | GYTFTNY |
| HCDR2 | 257 | Chothia | NPNTGE |
| HCDR3 | 171 | Chothia | SRGKNEAWFAY |
| HCDR1 | 258 | Kabat | NYGVN |
| HCDR2 | 170 | Kabat | WINPNTGEPTFDDDFKG |
| HCDR3 | 171 | Kabat | SRGKNEAWFAY |
| HCDR1 | 259 | IMGT | GYTFTNYG |
| HCDR2 | 260 | IMGT | INPNTGEP |
| HCDR3 | 261 | IMGT | SRSRGKNEAWFAY |
| HCDR1 | 169 | Combined | GYTFTNYGVN |
| HCDR2 | 170 | Combined | WINPNTGEPTFDDDFKG |
| HCDR3 | 171 | Combined | SRGKNEAWFAY |
| CD74 | 153 | Heavy | QVQLQQSGSELKKPGASVKVSCKASGYTFTNYGVNWI |
| Milatuzumab | | Chain (Fc | KQAPGQGLQWMGWINPNTGEPTFDDDFKGRFAFSLDT |
| DANAPA | | Silenced | SVSTAYLQISSLKADDTAVYFCSRSRGKNEAWFAYWG |
| Cysmab | | DANAPA) | QGTLVTVSS |
| HCDR1 | 256 | Chothia | GYTFTNY |
| HCDR2 | 257 | Chothia | NPNTGE |
| HCDR3 | 171 | Chothia | SRGKNEAWFAY |
| HCDR1 | 258 | Kabat | NYGVN |
| HCDR2 | 170 | Kabat | WINPNTGEPTFDDDFKG |
| HCDR3 | 171 | Kabat | SRGKNEAWFAY |
| HCDR1 | 259 | IMGT | GYTFTNYG |
| HCDR2 | 260 | IMGT | INPNTGEP |
| HCDR3 | 261 | IMGT | SRSRGKNEAWFAY |
| HCDR1 | 169 | Combined | GYTFTNYGVN |
| HCDR2 | 170 | Combined | WINPNTGEPTFDDDFKG |
| HCDR3 | 171 | Combined | SRGKNEAWFAY |
| CD74 | 262 | Light Chain | DIQLTQSPLSLPVTLGQPASISCRSSQSLVHRNGNTY |
| Milatuzumab | | | LHWFQQRPGQSPRLLIYTVSNRFSGVPDRFSGSGSGT |
| LC | | | DFTLKISRVEAEDVGVYFCSQSSHVPPTFGAGTRLEI |
| | | | K |
| LCDR1 | 263 | Chothia | SQSLVHRNGNTY |
| LCDR2 | 264 | Chothia | TVS |
| LCDR3 | 265 | Chothia | SSHVPP |
| LCDR1 | 266 | Kabat | RSSQSLVHRNGNTYLH |
| LCDR2 | 173 | Kabat | TVSNRFS |
| LCDR3 | 174 | Kabat | SQSSHVPPT |
| LCDR1 | 215 | IMGT | QSLVHRNGNTY |
| LCDR2 | 264 | IMGT | TVS |
| LCDR3 | 174 | IMGT | SQSSHVPPT |
| LCDR1 | 266 | Combined | RSSQSLVHRNGNTYLH |
| LCDR2 | 173 | Combined | TVSNRFS |
| LCDR3 | 174 | Combined | SQSSHVPPT |
| CD74 LC | 267 | Light Chain | DIVMTQTPLSLPVTPGEPASISCRSSQSLVHRNQNTY |
| Cysmab WT | | | LHWYLQKPGQSPQLLIYTVSNRFSGVPDRFSGSGSGT |
| (VHmil x | | | DFTLKSRVEAEDVGVYFCSQSSHVPPTFGQGTKLEIK |
| VKlaNQ) | | | |
| LCDR1 | 268 | Chothia | SQSLVHRNQNTY |
| LCDR2 | 264 | Chothia | TVS |
| LCDR3 | 265 | Chothia | SSHVPP |
| LCDR1 | 172 | Kabat | RSSQSLVHRNQNTYLH |
| LCDR2 | 173 | Kabat | TVSNRFS |
| LCDR3 | 174 | Kabat | SQSSHVPPT |
| LCDR1 | 269 | IMGT | QSLVHRNQNTY |
| LCDR2 | 264 | IMGT | TVS |
| LCDR3 | 174 | IMGT | SQSSHVPPT |
| LCDR1 | 172 | Combined | RSSQSLVHRNQNTYLH |
| LCDR2 | 173 | Combined | TVSNRFS |
| LCDR3 | 174 | Combined | SQSSHVPPT |
| CD48 NY920 | 270 | Heavy | EVQLLESGGGLVQPGGSLRLSCAASGFTFSSFAMSWV |
| Cysmab | | Chain | RQAPGKGLEWVSAISGFGGSTYYADSVKGRFTISRDN |
| WT HC | | (Wild Type | SKNTLYLQMNSLRAEDTAVYYCARQFWEDQPFYFDYW |
| | | Fc) | GQGTLVTVSS |
| HCDR1 | 271 | Chothia | GFTFSSF |
| HCDR2 | 272 | Chothia | SGFGGS |
| HCDR3 | 273 | Chothia | QFWEDQPFYFDY |
| HCDR1 | 274 | Kabat | SFAMS |
| HCDR2 | 275 | Kabat | AISGFGGSTYYADSVKG |
| HCDR3 | 273 | Kabat | QFWEDQPFYFDY |
| HCDR1 | 276 | IMGT | GFTFSSFA |
| HCDR2 | 277 | IMGT | ISGFGGST |
| HCDR3 | 278 | IMGT | ARQFWEDQPFYFDY |
| HCDR1 | 279 | Combined | GFTFSSFAMS |
| HCDR2 | 275 | Combined | AISGFGGSTYYADSVKG |
| HCDR3 | 273 | Combined | QFWEDQPFYFDY |
| CD48 NY920 | 270 | | EVQLLESGGGLVQPGGSLRLSCAASGFTFSSFAMSWV |
| Cysmab DAPA | | | RQAPGKGLEWVSAISGFGGSTYYADSVKGRFTISRDN |
| HC | | | SKNTLYLQMNSLRAEDTAVYYCARQFWEDQPFYFDYW |
| | | | GQGTLVTVSS |
| HCDR1 | 271 | Chothia | GFTFSSF |
| HCDR2 | 272 | Chothia | SGFGGS |
| HCDR3 | 273 | Chothia | QFWEDQPFYFDY |
| HCDR1 | 274 | Kabat | SFAMS |
| HCDR2 | 275 | Kabat | AISGFGGSTYYADSVKG |
| HCDR3 | 273 | Kabat | QFWEDQPFYFDY |
| HCDR1 | 276 | IMGT | GFTFSSFA |
| HCDR2 | 277 | IMGT | ISGFGGST |
| HCDR3 | 278 | IMGT | ARQFWEDQPFYFDY |
| HCDR1 | 279 | Combined | GFTFSSFAMS |
| HCDR2 | 275 | Combined | AISGFGGSTYYADSVKG |
| HCDR3 | 273 | Combined | QFWEDQPFYFDY |
| CD48 NY920 | 280 | Light Chain | DIQMTQSPSSLSASVGDRVTITCRASQSISSYLNWYQ |
| LC | | | QKPGKAPKLLIYAASSLQSGVPSRFSGSGSGTDFTLT |
| | | | ISSLQPEDFATYYCQQSYSTPLTFGQGTKVEIK |
| LCDR1 | 281 | Chothia | SQSISSY |
| LCDR2 | 282 | Chothia | AAS |
| LCDR3 | 283 | Chothia | SYSTPL |
| LCDR1 | 284 | Kabat | RASQSISSYLN |
| LCDR2 | 285 | Kabat | AASSLQS |
| LCDR3 | 286 | Kabat | QQSYSTPLT |
| LCDR1 | 287 | IMGT | QSISSY |
| LCDR2 | 282 | IMGT | AAS |
| LCDR3 | 286 | IMGT | QQSYSTPLT |
| LCDR1 | 284 | Combined | RASQSISSYLN |
| LCDR2 | 288 | Combined | AASSLQS |
| LCDR3 | 286 | Combined | QQSYSTPLT |
| Her2 Cysmab | 9 | Heavy | EVQLVESGGGLVQPGGSLRLSCAASGFNIKDTYIHWV |
| WT HC | | Chain | RQAPGKGLEWVARIYPTNGYTRYADSVKGRFTISADT |
| | | (Wild Type | SKNTAYLQMNSLRAEDTAVYYCSRWGGDGFYAMDYWG |
| | | Fc) | QGTLVTVSS |
| HCDR1 | 289 | Chothia | GFNIKDT |
| HCDR2 | 290 | Chothia | YPTNGY |
| HCDR3 | 291 | Chothia | WGGDGFYAMDY |
| HCDR1 | 292 | Kabat | DTYIH |
| HCDR2 | 40 | Kabat | RIYPTNGYTRYADSVKG |
| HCDR3 | 291 | Kabat | WGGDGFYAMDY |
| HCDR1 | 293 | IMGT | GFNIKDTY |
| HCDR2 | 294 | IMGT | IYPTNGYT |
| HCDR3 | 295 | IMGT | SRWGGDGFYAMDY |
| HCDR1 | 39 | Combined | GFNIKDTYIH |
| HCDR2 | 40 | Combined | RIYPTNGYTRYADSVKG |
| HCDR3 | 291 | Combined | WGGDGFYAMDY |
| Her2 LC | 296 | Light Chain | DIQMTQSPSSLSASVGDRVTITCRASQDVNTAVAWYQ |
| | | | QKPGKAPKLLIYSASFLYSGVPSRFSGSRSGTDFTLT |
| | | | ISSLQPEDFATYYCQQHYTTPPTFGQGTKVEIK |
| LCDR1 | 297 | Chothia | SQDVNTA |
| LCDR2 | 298 | Chothia | SAS |
| LCDR3 | 299 | Chothia | HYTTPP |
| LCDR1 | 300 | Kabat | RASQDVNTAVA |
| LCDR2 | 301 | Kabat | SASFLYS |
| LCDR3 | 44 | Kabat | QQHYTTPPT |
| LCDR1 | 302 | IMGT | QDVNTA |
| LCDR2 | 298 | IMGT | SAS |
| LCDR3 | 44 | IMGT | QQHYTTPPT |
| LCDR1 | 300 | Combined | RASQDVNTAVA |
| LCDR2 | 301 | Combined | SASFLYS |
| LCDR3 | 44 | Combined | QQHYTTPPT |
| PCAD CQY679 | 303 | Heavy | EVQLVQSGAEVKKPGESLKISCKVSGYTFTDHTIHWM |
| Cysmab | | Chain | RQMPGKGLEWMGYIYPRSGSINYNEKFKGQVTISADK |
| WT HC | | (Wild Type | SSSTAYLQWSSLKASDTAMYYCARRNLFLPMEYWGQG |
| | | Fc) | TLVTVSS |
| HCDR1 | 304 | Chothia | GYTFTDH |
| HCDR2 | 305 | Chothia | YPRSGS |
| HCDR3 | 306 | Chothia | RNLFLPMEY |
| HCDR1 | 307 | Kabat | DHTIH |
| HCDR2 | 308 | Kabat | YIYPRSGSINYNEKFKG |
| HCDR3 | 306 | Kabat | RNLFLPMEY |
| HCDR1 | 309 | IMGT | GYTFTDHT |
| HCDR2 | 277 | IMGT | ISGFGGST |
| HCDR3 | 278 | IMGT | ARQFWEDQPFYFDY |
| HCDR1 | 310 | Combined | GYTFTDHTIH |
| HCDR2 | 308 | Combined | YIYPRSGSINYNEKFKG |
| HCDR3 | 306 | Combined | RNLFLPMEY |
| PCAD CQY679 | 311 | Light Chain | DIVMTQTPLSLPVTPGEPASISCRSSQSLLSSGDQKN |
| LC | | | YLTWYLQKPGQSPQLLIYWASTRESGVPDRFSGSGSG |
| | | | TDFTLKISRVEAEDVGVYYCQNDYRYPLTFGQGTKLE |
| | | | IK |
| LCDR1 | 312 | Chothia | SQSLLSSGDQKNY |
| LCDR2 | 313 | Chothia | WAS |
| LCDR3 | 314 | Chothia | DYRYPL |
| LCDR1 | 315 | Kabat | RSSQSLLSSGDQKNYLT |
| LCDR2 | 25 | Kabat | WASTRES |
| LCDR3 | 316 | Kabat | QNDYRYPLT |
| LCDR1 | 317 | IMGT | QSLLSSGDQKNY |
| LCDR2 | 313 | IMGT | WAS |
| LCDR3 | 316 | IMGT | QNDYRYPLT |
| LCDR1 | 315 | Combined | RSSQSLLSSGDQKNYLT |
| LCDR2 | 25 | Combined | WASTRES |
| LCDR3 | 316 | Combined | QNDYRYPLT |
| EphA2 1C1 | 318 | Heavy | EVQLLESGGGLVQPGGSLRLSCAASGFTFSHYMMAWV |
| Cysmab | | Chain | RQAPGKGLEWVSRIGPSGGPTHYADSVKGRFTISRDN |
| WT HC | | (Wild Type | SKNTLYLQMNSLRAEDTAVYYCAGYDSGYDYVAVAGP |
| | | Fc) | AEYFQHWGQGTLVTVSS |
| HCDR1 | 319 | Chothia | GFTFSHY |
| HCDR2 | 320 | Chothia | GPSGGP |
| HCDR3 | 321 | Chothia | YDSGYDYVAVAGPAEYFQH |
| HCDR1 | 322 | Kabat | HYMMA |
| HCDR2 | 323 | Kabat | RIGPSGGPTHYADSVKG |
| HCDR3 | 324 | Kabat | YDSGYDYVAVAGPAEYFQH |
| HCDR1 | 325 | IMGT | GFTFSHYM |
| HCDR2 | 326 | IMGT | IGPSGGPT |
| HCDR3 | 327 | IMGT | AGYDSGYDYVAVAGPAEYFQH |
| HCDR1 | 328 | Combined | GFTFSHYMMA |
| HCDR2 | 323 | Combined | RIGPSGGPTHYADSVKG |
| HCDR3 | 321 | Combined | YDSGYDYVAVAGPAEYFQH |
| EphA2 LC | 329 | Light Chain | DIQMTQSPSSLSASVGDRVTITCRASQSISTWLAWYQ |
| | | | QKPGKAPKLLIYKASNLHTGVPSRFSGSGSGTEFSLT |
| | | | ISGLQPDDFATYYCQQYNSYSRTFGQGTKVEIK |
| LCDR1 | 330 | Chothia | SQSISTW |
| LCDR2 | 331 | Chothia | KAS |
| LCDR3 | 332 | Chothia | YNSYSR |
| LCDR1 | 333 | Kabat | RASQSISTWLA |
| LCDR2 | 334 | Kabat | KASNLHT |
| LCDR3 | 335 | Kabat | QQYNSYSRT |
| LCDR1 | 336 | IMGT | QSISTW |
| LCDR2 | 331 | IMGT | KAS |
| LCDR3 | 335 | IMGT | QQYNSYSRT |
| LCDR1 | 333 | Combined | RASQSISTWLA |
| LCDR2 | 334 | Combined | KASNLHT |
| LCDR3 | 335 | Combined | QQYNSYSRT |
| HER2 | | | |
| Disitamab | | | |
| HCDR1 | 358 | Combined | GYTFTDYYIH |
| HCDR2 | 359 | Combined | RVNPDHGDSYYNQKFKD |
| HCDR3 | 360 | Combined | NYLFDH |
| HCDR1 | 361 | Kabat | DYYIH |
| HCDR2 | 359 | Kabat | RVNPDHGDSYYNQKFKD |
| HCDR3 | 360 | Kabat | NYLFDH |
| HCDR1 | 361 | Chothia | GYTFTDY |
| HCDR2 | 362 | Chothia | NPDHGD |
| HCDR3 | 363 | Chothia | NYLFDH |
| HCDR1 | 364 | IMGT | GYTFTDYY |
| HCDR2 | 365 | IMGT | VNPDHGDS |
| HCDR3 | 366 | IMGT | ARNYLFDH |
| HER2 | | | |
| Disitamab | | | |
| LCDR1 | 367 | Combined | KASQDVGTAVA |
| LCDR2 | 368 | Combined | WASIRHT |
| LCDR3 | 369 | Combined | HQFATYT |
| LCDR1 | 367 | Kabat | KASQDVGTAVA |
| LCDR2 | 368 | Kabat | WASIRHT |
| LCDR3 | 369 | Kabat | HQFATYT |
| LCDR1 | 370 | Chothia | SQDVGTA |
| LCDR2 | 313 | Chothia | WAS |
| LCDR3 | 371 | Chothia | FATY |
| LCDR1 | 372 | IMGT | QDVGTA |
| LCDR2 | 313 | IMGT | WAS |
| LCDR3 | 369 | IMGT | HQFATYT |
| 5T4 | | | |
| HCDR1 | 373 | Combined | GYTFTNFGMN |
| HCDR2 | 374 | Combined | WINTNTGEPRYAEEFKG |
| HCDR3 | 375 | Combined | DWDGAYFFDY |
| HCDR1 | 376 | Chothia | GYTFTNF |
| HCDR2 | 377 | Chothia | NTNTGE |
| HCDR3 | 375 | Chothia | DWDGAYFFDY |
| HCDR1 | 378 | Kabat | NFGMN |
| HCDR2 | 374 | Kabat | WINTNTGEPRYAEEFKG |
| HCDR3 | 375 | Kabat | DWDGAYFFDY |
| HCDR1 | 376 | IMGT | GYTFTNFG |
| HCDR2 | 379 | IMGT | INTNTGEP |
| HCDR3 | 380 | IMGT | ARDWDGAYFFDY |
| 5T4 | | | |
| LCDR1 | 381 | Combined | KASQSVSNDVA |
| LCDR2 | 382 | Combined | FATNRYT |
| LCDR3 | 383 | Combined | QQDYSSPWT |
| LCDR1 | 384 | Chothia | SQSVSND |
| LCDR2 | 385 | Chothia | FAT |
| LCDR3 | 386 | Chothia | DYSSPW |
| LCDR1 | 381 | Kabat | KASQSVSNDVA |
| LCDR2 | 382 | Kabat | FATNRYT |
| LCDR3 | 383 | Kabat | QQDYSSPWT |
| LCDR1 | 387 | IMGT | QSVSND |
| LCDR2 | 385 | IMGT | FAT |
| LCDR1 | 383 | IMGT | QQDYSSPWT |
| Trop2 | | | |
| Datopotamab | | | |
| HCDR1 | 388 | Combined | GYTFTTAGMQ |
| HCDR2 | 389 | Combined | WINTHSGVPKYAEDFKG |
| HCDR3 | 390 | Combined | SGFGSSYWYFDV |
| HCDR1 | 402 | Chothia | GYTFTTA |
| HCDR2 | 392 | Chothia | NTHSGV |
| HCDR3 | 390 | Chothia | SGFGSSYWYFDV |
| HCDR1 | 393 | Kabat | TAGMQ |
| HCDR2 | 389 | Kabat | WINTHSGVPKYAEDFKG |
| HCDR3 | 390 | Kabat | SGFGSSYWYFDV |
| HCDR1 | 391 | IMGT | GYTFTTAG |
| HCDR2 | 394 | IMGT | INTHSGVP |
| HCDR3 | 395 | IMGT | ARSGFGSSYWYFDV |
| Trop2 | | | |
| Datopotamab | | | |
| LCDR1 | 396 | Combined | KASQDVSTAVA |
| LCDR2 | 397 | Combined | SASYRYT |
| LCDR3 | 398 | Combined | QQHYITPLT |
| LCDR1 | 399 | Chothia | SQDVSTA |
| LCDR2 | 298 | Chothia | SAS |
| LCDR3 | 400 | Chothia | HYITPL |
| LCDR1 | 396 | Kabat | KASQDVSTAVA |
| LCDR2 | 397 | Kabat | SASYRYT |
| LCDR3 | 398 | Kabat | QQHYITPLT |
| LCDR1 | 401 | IMGT | QDVSTA |
| LCDR2 | 298 | IMGT | SAS |
| LCDR1 | 398 | IMGT | QQHYITPLT |

In some embodiments, the antibody or antigen-binding fragment of an ADC disclosed herein may comprise any set of heavy and light chain variable domains listed in the tables above or a set of six CDRs from any set of heavy and light chain variable domains listed in the tables above. In some embodiments, the antibody or antigen-binding fragment of an ADC disclosed herein may comprise amino acid sequences that are conservatively modified and/or homologous to the sequences listed in the tables above, so long as the ADC retains the ability to bind to its target cancer antigen (e.g., with a KID of less than 1×10⁻⁸ M) and retains one or more functional properties of the ADCs disclosed herein (e.g., ability to internalize, bind to an antigen target, e.g., an antigen expressed on a tumor or other cancer cell, etc.).

In some embodiments, the antibody or antigen-binding fragment of an ADC disclosed herein further comprises human heavy and light chain constant domains or fragments thereof. For instance, the antibody or antigen-binding fragment of the described ADCs may comprise a human IgG heavy chain constant domain (such as an IgG1) and a human kappa or lambda light chain constant domain. In some embodiments, the antibody or antigen-binding fragment of the described ADCs comprises a human immunoglobulin G subtype 1 (IgG1) heavy chain constant domain with a human Ig kappa light chain constant domain.

In some embodiments, the target cancer antigen for an ADC is PCAD.

In some embodiments, the anti-PCAD antibody or antigen-binding fragment thereof comprises three heavy chain CDRs and three light chain CDRs as follows: heavy chain CDR1 (HCDR1) consisting of SEQ ID NO:33, heavy chain CDR2 (HCDR2) consisting of SEQ ID NO:34, heavy chain CDR3 (HCDR3) consisting of SEQ ID NO:35; light chain CDR1 (LCDR1) consisting of SEQ ID NO:36, light chain CDR2 (LCDR2) consisting of SEQ ID NO:37, and light chain CDR3 (LCDR3) consisting of SEQ ID NO:38.

In some embodiments, the anti-PCAD antibody or antigen-binding fragment thereof comprises three heavy chain CDRs and three light chain CDRs as follows: heavy chain CDR1 (HCDR1) consisting of SEQ ID NO:304, heavy chain CDR2 (HCDR2) consisting of SEQ ID NO:305, heavy chain CDR3 (HCDR3) consisting of SEQ ID NO:306; light chain CDR1 (LCDR1) consisting of SEQ ID NO:312, light chain CDR2 (LCDR2) consisting of SEQ ID NO:313, and light chain CDR3 (LCDR3) consisting of SEQ ID NO:314.

In some embodiments, the anti-PCAD antibody or antigen-binding fragment thereof comprises three heavy chain CDRs and three light chain CDRs as follows: heavy chain CDR1 (HCDR1) consisting of SEQ ID NO:307, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316.

In some embodiments, the anti-PCAD antibody or antigen-binding fragment thereof comprises three heavy chain CDRs and three light chain CDRs as follows: heavy chain CDR1 (HCDR1) consisting of SEQ ID NO:309, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:317, light chain CDR2 (LCDR2) consisting of SEQ ID NO:313, and light chain CDR3 (LCDR3) consisting of SEQ ID NO:316.

In some embodiments, the anti-PCAD antibody or antigen-binding fragment thereof comprises three heavy chain CDRs and three light chain CDRs as follows: heavy chain CDR1 (HCDR1) consisting of SEQ ID NO:310, heavy chain CDR2 (HCDR2) consisting of SEQ ID NO:308, heavy chain CDR3 (HCDR3) consisting of SEQ ID NO:306; light chain CDR1 (LCDR1) consisting of SEQ ID NO:315, light chain CDR2 (LCDR2) consisting of SEQ ID NO:25, and light chain CDR3 (LCDR3) consisting of SEQ ID NO:316.

In some embodiments, the anti-PCAD antibody or antigen-binding fragment thereof comprises a heavy chain variable region comprising the amino acid sequence of SEQ ID NO:7, and a light chain variable region comprising the amino acid sequence of SEQ ID NO:8. In some embodiments, the anti-PCAD antibody or antigen-binding fragment thereof comprises the heavy chain variable region amino acid sequence of SEQ ID NO:7 and the light chain variable region amino acid sequence of SEQ ID NO:8, or sequences that are at least 95% identical to the disclosed sequences. In some embodiments, the anti-PCAD antibody or antigen-binding fragment thereof has a heavy chain variable region amino acid sequence that is at least 96%, at least 97%, at least 98%, or at least 99% identical to SEQ ID NO:7 and/or a light chain variable region amino acid sequence that is at least 96%, at least 97%, at least 98%, or at least 99% identical to SEQ ID NO:8.

In some embodiments, the anti-PCAD antibody or antigen-binding fragment thereof comprises a heavy chain variable region comprising the amino acid sequence of SEQ ID NO:303, and a light chain variable region comprising the amino acid sequence of SEQ ID NO:311. In some embodiments, the anti-PCAD antibody or antigen-binding fragment thereof comprises the heavy chain variable region amino acid sequence of SEQ ID NO:303 and the light chain variable region amino acid sequence of SEQ ID NO:311, or sequences that are at least 95% identical to the disclosed sequences. In some embodiments, the anti-PCAD antibody or antigen-binding fragment thereof has a heavy chain variable region amino acid sequence that is at least 96%, at least 97%, at least 98%, or at least 99% identical to SEQ ID NO:303 and/or a light chain variable region amino acid sequence that is at least 96%, at least 97%, at least 98%, or at least 99% identical to SEQ ID NO:311.

In some embodiments, the anti-PCAD antibody or antigen-binding fragment thereof is an internalizing antibody or internalizing antigen-binding fragment. In some embodiments, the anti-PCAD antibody comprises a human IgG1 heavy chain constant domain or a modified IgG1 heavy chain constant domain. In some embodiments, the IgG1 heavy chain constant domain comprises a cysteine residue (C) at the amino acid positions corresponding to 152 and 375 in a wild-type (unmodified) IgG1 heavy chain constant domain numbered according to EU numbering system.

In some embodiments, the anti-PCAD antibody comprises the heavy chain amino acid sequence of SEQ ID NO:63 or a sequence that is at least 95% identical to SEQ ID NO:63, and the light chain amino acid sequence of SEQ ID NO:64 or a sequence that is at least 95% identical to SEQ ID NO:64. In some embodiments, the anti-PCAD antibody comprises the heavy chain amino acid sequence of SEQ ID NO:63 and the light chain amino acid sequence of SEQ ID NO:64, or sequences that are at least 95% identical to the disclosed sequences. In some embodiments, the anti-PCAD antibody has a heavy chain amino acid sequence that is at least 96%, at least 97%, at least 98%, or at least 99% identical to SEQ ID NO:63 and a light chain amino acid sequence that is at least 96%, at least 97%, at least 98%, or at least 99% identical to SEQ ID NO:64. In some embodiments, the anti-PCAD antibody is NOV169N31Q (WO 2016/203432), or an antigen-binding fragment thereof.

In some embodiments, the anti-PCAD antibody comprises the heavy chain amino acid sequence of SEQ ID NO:248 or a sequence that is at least 95% identical to SEQ ID NO:248, and the light chain amino acid sequence of SEQ ID NO:250 or a sequence that is at least 95% identical to SEQ ID NO:250. In some embodiments, the anti-PCAD antibody comprises the heavy chain amino acid sequence of SEQ ID NO:248 and the light chain amino acid sequence of SEQ ID NO:250, or sequences that are at least 95% identical to the disclosed sequences. In some embodiments, the anti-PCAD antibody has a heavy chain amino acid sequence that is at least 96%, at least 97%, at least 98%, or at least 99% identical to SEQ ID NO:248 and a light chain amino acid sequence that is at least 96%, at least 97%, at least 98%, or at least 99% identical to SEQ ID NO:250.

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:304), HCDR2 (SEQ ID NO:305), HCDR3 (SEQ ID NO:306); LCDR1 (SEQ ID NO:312), LCDR2 (SEQ ID NO:313), and LCDR3 (SEQ ID NO:314).

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:307), HCDR2 (SEQ ID NO:308), HCDR3 (SEQ ID NO:306); LCDR1 (SEQ ID NO:315), LCDR2 (SEQ ID NO:25), and LCDR3 (SEQ ID NO:316).

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:309), HCDR2 (SEQ ID NO:277), HCDR3 (SEQ ID NO:278); LCDR1 (SEQ ID NO:317), LCDR2 (SEQ ID NO:313), and LCDR3 (SEQ ID NO:316).

In some embodiments, the anti-PCAD antibody or antigen-binding fragment thereof comprises the three heavy chain CDRs and three light chain CDRs of CQY679 or wherein the CDRs include no more than one, two, three, four, five, or six amino acid additions, deletions or substitutions of HCDR1 (SEQ ID NO:310), HCDR2 (SEQ ID NO:308), HCDR3 (SEQ ID NO:306); LCDR1 (SEQ ID NO:315), LCDR2 (SEQ ID NO:25), and LCDR3 (SEQ ID NO:316).

In some embodiments, the anti-PCAD antibody or antigen-binding fragment thereof comprises the three heavy chain CDRs and three light chain CDRs of NOV169N31Q or wherein the CDRs include no more than one, two, three, four, five, or six amino acid additions, deletions or substitutions of HCDR1 (SEQ ID NO:33), HCDR2 (SEQ ID NO:34), HCDR3 (SEQ ID NO:35); LCDR1 (SEQ ID NO:36), LCDR2 (SEQ ID NO:37), and LCDR3 (SEQ ID NO:38).

In some embodiments, the target cancer antigen for an ADC is HER2.

In some embodiments, the anti-HER2 antibody or antigen-binding fragment thereof comprises three heavy chain CDRs and three light chain CDRs as follows: heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:41; light chain CDR1 (LCDR1) consisting of SEQ ID NO:42, light chain CDR2 (LCDR2) consisting of SEQ ID NO:43, and light chain CDR3 (LCDR3) consisting of SEQ ID NO:44.

In some embodiments, the anti-HER2 antibody or antigen-binding fragment thereof comprises three heavy chain CDRs and three light chain CDRs as follows: heavy chain CDR1 (HCDR1) consisting of SEQ ID NO:289, heavy chain CDR2 (HCDR2) consisting of SEQ ID NO:290, heavy chain CDR3 (HCDR3) consisting of SEQ ID NO:291; light chain CDR1 (LCDR1) consisting of SEQ ID NO:297, light chain CDR2 (LCDR2) consisting of SEQ ID NO:298, and light chain CDR3 (LCDR3) consisting of SEQ ID NO:299.

In some embodiments, the anti-HER2 antibody or antigen-binding fragment thereof comprises three heavy chain CDRs and three light chain CDRs as follows: heavy chain CDR1 (HCDR1) consisting of SEQ ID NO:292, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44.

In some embodiments, the anti-HER2 antibody or antigen-binding fragment thereof comprises three heavy chain CDRs and three light chain CDRs as follows: heavy chain CDR1 (HCDR1) consisting of SEQ ID NO:293, heavy chain CDR2 (HCDR2) consisting of SEQ ID NO:294, heavy chain CDR3 (HCDR3) consisting of SEQ ID NO:295; light chain CDR1 (LCDR1) consisting of SEQ ID NO:302, light chain CDR2 (LCDR2) consisting of SEQ ID NO:298, and light chain CDR3 (LCDR3) consisting of SEQ ID NO:44.

In some embodiments, the anti-HER2 antibody or antigen-binding fragment thereof comprises three heavy chain CDRs and three light chain CDRs as follows: heavy chain CDR1 (HCDR1) consisting of SEQ ID NO:39, heavy chain CDR2 (HCDR2) consisting of SEQ ID NO:40, heavy chain CDR3 (HCDR3) consisting of SEQ ID NO:291; light chain CDR1 (LCDR1) consisting of SEQ ID NO:300, light chain CDR2 (LCDR2) consisting of SEQ ID NO:301, and light chain CDR3 (LCDR3) consisting of SEQ ID NO:44.

In some embodiments, the anti-HER2 antibody or antigen-binding fragment thereof comprises a heavy chain variable region comprising the amino acid sequence of SEQ ID NO:9, and a light chain variable region comprising the amino acid sequence of SEQ ID NO:10. In some embodiments, the anti-HER2 antibody or antigen-binding fragment thereof comprises the heavy chain variable region amino acid sequence of SEQ ID NO:9 and the light chain variable region amino acid sequence of SEQ ID NO:10, or sequences that are at least 95% identical to the disclosed sequences. In some embodiments, the anti-HER2 antibody or antigen-binding fragment thereof has a heavy chain variable region amino acid sequence that is at least 96%, at least 97%, at least 98%, or at least 99% identical to SEQ ID NO:9 and/or a light chain variable region amino acid sequence that is at least 96%, at least 97%, at least 98%, or at least 99% identical to SEQ ID NO:10.

In some embodiments, the anti-HER2 antibody or antigen-binding fragment thereof comprises a heavy chain variable region comprising the amino acid sequence of SEQ ID NO:9, and a light chain variable region comprising the amino acid sequence of SEQ ID NO:296. In some embodiments, the anti-HER2 antibody or antigen-binding fragment thereof comprises the heavy chain variable region amino acid sequence of SEQ ID NO:9 and the light chain variable region amino acid sequence of SEQ ID NO:296, or sequences that are at least 95% identical to the disclosed sequences. In some embodiments, the anti-HER2 antibody or antigen-binding fragment thereof has a heavy chain variable region amino acid sequence that is at least 96%, at least 97%, at least 98%, or at least 99% identical to SEQ ID NO:9 and/or a light chain variable region amino acid sequence that is at least 96%, at least 97%, at least 98%, or at least 99% identical to SEQ ID NO:296.

In some embodiments, the anti-HER2 antibody or antigen-binding fragment thereof is an internalizing antibody or internalizing antigen-binding fragment. In some embodiments, the anti-HER2 antibody comprises a human IgG1 heavy chain constant domain or a modified IgG1 heavy chain constant domain. In some embodiments, the IgG1 heavy chain constant domain comprises a glutamine residue (Q) at the amino acid position corresponding to 297 in a wild-type (unmodified) IgG1 heavy chain constant domain. In some embodiments, the IgG1 heavy chain constant domain comprises a serine residue (S) at the amino acid position corresponding to 297 in a wild-type (unmodified) IgG1 heavy chain constant domain. In some embodiments, the IgG1 heavy chain constant domain comprises a cysteine residue (C) at the amino acid positions corresponding to 152 and 375 in a wild-type (unmodified) IgG1 heavy chain constant domain numbered according to EU numbering system. In some embodiments, the anti-HER2 antibody comprises a human Ig kappa light chain constant domain or a modified Ig kappa light chain constant domain.

In some embodiments, the anti-HER2 antibody comprises the heavy chain amino acid sequence of SEQ ID NO:65 or a sequence that is at least 95% identical to SEQ ID NO:65, and the light chain amino acid sequence of SEQ ID NO:66 or a sequence that is at least 95% identical to SEQ ID NO:66. In some embodiments, the anti-HER2 antibody comprises the heavy chain amino acid sequence of SEQ ID NO:65 and the light chain amino acid sequence of SEQ ID NO:66, or sequences that are at least 95% identical to the disclosed sequences. In some embodiments, the anti-HER2 antibody has a heavy chain amino acid sequence that is at least 96%, at least 97%, at least 98%, or at least 99% identical to SEQ ID NO:65 and a light chain amino acid sequence that is at least 96%, at least 97%, at least 98%, or at least 99% identical to SEQ ID NO:66. In some embodiments, the anti-HER2 antibody is trastuzumab (U.S. Pat. Nos. 5,821,337 and 6,870,034; see also Molina et al. (2001) Cancer Res. 61(12):4744-9), or an antigen-binding fragment thereof.

In some embodiments, the anti-HER2 antibody comprises the heavy chain amino acid sequence of SEQ ID NO:245 or a sequence that is at least 95% identical to SEQ ID NO:245, and the light chain amino acid sequence of SEQ ID NO:66 or a sequence that is at least 95% identical to SEQ ID NO:66. In some embodiments, the anti-HER2 antibody comprises the heavy chain amino acid sequence of SEQ ID NO:245 and the light chain amino acid sequence of SEQ ID NO:66, or sequences that are at least 95% identical to the disclosed sequences. In some embodiments, the anti-HER2 antibody has a heavy chain amino acid sequence that is at least 96%, at least 97%, at least 98%, or at least 99% identical to SEQ ID NO:245 and a light chain amino acid sequence that is at least 96%, at least 97%, at least 98%, or at least 99% identical to SEQ ID NO:66.

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:39), HCDR2 (SEQ ID NO:40), HCDR3 (SEQ ID NO:41); LCDR1 (SEQ ID NO:42), LCDR2 (SEQ ID NO:43), and LCDR3 (SEQ ID NO:44).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:289), HCDR2 (SEQ ID NO:290), HCDR3 (SEQ ID NO:291); LCDR1 (SEQ ID NO:297), LCDR2 (SEQ ID NO:298), and LCDR3 (SEQ ID NO:299).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:292), HCDR2 (SEQ ID NO:40), HCDR3 (SEQ ID NO:291); LCDR1 (SEQ ID NO:300), LCDR2 (SEQ ID NO:301), and LCDR3 (SEQ ID NO:44).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:293), HCDR2 (SEQ ID NO:294), HCDR3 (SEQ ID NO:295); LCDR1 (SEQ ID NO:302), LCDR2 (SEQ ID NO:298), and LCDR3 (SEQ ID NO:44).

In some embodiments, the anti-HER2 antibody or antigen-binding fragment thereof comprises the three heavy chain CDRs and three light chain CDRs of trastuzumab or wherein the CDRs include no more than one, two, three, four, five, or six amino acid additions, deletions or substitutions of HCDR1 (SEQ ID NO:39), HCDR2 (SEQ ID NO:40), HCDR3 (SEQ ID NO:291); LCDR1 (SEQ ID NO:300), LCDR2 (SEQ ID NO:301), and LCDR3 (SEQ ID NO:44).

In some embodiments, the target cancer antigen for an ADC is CD48.

In some embodiments, the anti-CD48 antibody or antigen-binding fragment thereof comprises three heavy chain CDRs and three light chain CDRs as follows: heavy chain CDR1 (HCDR1) consisting of SEQ ID NO:51, heavy chain CDR2 (HCDR2) consisting of SEQ ID NO:52, heavy chain CDR3 (HCDR3) consisting of SEQ ID NO:53; light chain CDR1 (LCDR1) consisting of SEQ ID NO:54, light chain CDR2 (LCDR2) consisting of SEQ ID NO:55, and light chain CDR3 (LCDR3) consisting of SEQ ID NO:56.

In some embodiments, the anti-CD48 antibody or antigen-binding fragment thereof comprises three heavy chain CDRs and three light chain CDRs as follows: heavy chain CDR1 (HCDR1) consisting of SEQ ID NO:271, heavy chain CDR2 (HCDR2) consisting of SEQ ID NO:272, heavy chain CDR3 (HCDR3) consisting of SEQ ID NO:273; light chain CDR1 (LCDR1) consisting of SEQ ID NO:281, light chain CDR2 (LCDR2) consisting of SEQ ID NO:282, and light chain CDR3 (LCDR3) consisting of SEQ ID NO:283.

In some embodiments, the anti-CD48 antibody or antigen-binding fragment thereof comprises three heavy chain CDRs and three light chain CDRs as follows: heavy chain CDR1 (HCDR1) consisting of SEQ ID NO:274, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:285, and light chain CDR3 (LCDR3) consisting of SEQ ID NO:286.

In some embodiments, the anti-CD48 antibody or antigen-binding fragment thereof comprises three heavy chain CDRs and three light chain CDRs as follows: heavy chain CDR1 (HCDR1) consisting of SEQ ID NO:276, heavy chain CDR2 (HCDR2) consisting of SEQ ID NO:277, heavy chain CDR3 (HCDR3) consisting of SEQ ID NO:278; light chain CDR1 (LCDR1) consisting of SEQ ID NO:287, light chain CDR2 (LCDR2) consisting of SEQ ID NO:282, and light chain CDR3 (LCDR3) consisting of SEQ ID NO:286.

In some embodiments, the anti-CD48 antibody or antigen-binding fragment thereof comprises three heavy chain CDRs and three light chain CDRs as follows: heavy chain CDR1 (HCDR1) consisting of SEQ ID NO:279, heavy chain CDR2 (HCDR2) consisting of SEQ ID NO:275, heavy chain CDR3 (HCDR3) consisting of SEQ ID NO:273; light chain CDR1 (LCDR1) consisting of SEQ ID NO:284, light chain CDR2 (LCDR2) consisting of SEQ ID NO:288, and light chain CDR3 (LCDR3) consisting of SEQ ID NO:286.

In some embodiments, the anti-CD48 antibody or antigen-binding fragment thereof comprises a heavy chain variable region comprising the amino acid sequence of SEQ ID NO:13, and a light chain variable region comprising the amino acid sequence of SEQ ID NO:14. In some embodiments, the anti-CD48 antibody or antigen-binding fragment thereof comprises the heavy chain variable region amino acid sequence of SEQ ID NO:13 and the light chain variable region amino acid sequence of SEQ ID NO:14, or sequences that are at least 95% identical to the disclosed sequences. In some embodiments, the anti-CD48 antibody or antigen-binding fragment thereof has a heavy chain variable region amino acid sequence that is at least 96%, at least 97%, at least 98%, or at least 99% identical to SEQ ID NO:13 and/or a light chain variable region amino acid sequence that is at least 96%, at least 97%, at least 98%, or at least 99% identical to SEQ ID NO:14.

In some embodiments, the anti-CD48 antibody or antigen-binding fragment thereof comprises a heavy chain variable region comprising the amino acid sequence of SEQ ID NO:270, and a light chain variable region comprising the amino acid sequence of SEQ ID NO:280. In some embodiments, the anti-CD48 antibody or antigen-binding fragment thereof comprises the heavy chain variable region amino acid sequence of SEQ ID NO:270 and the light chain variable region amino acid sequence of SEQ ID NO:280, or sequences that are at least 95% identical to the disclosed sequences. In some embodiments, the anti-CD48 antibody or antigen-binding fragment thereof has a heavy chain variable region amino acid sequence that is at least 96%, at least 97%, at least 98%, or at least 99% identical to SEQ ID NO:270 and/or a light chain variable region amino acid sequence that is at least 96%, at least 97%, at least 98%, or at least 99% identical to SEQ ID NO:280.

In some embodiments, the anti-CD48 antibody or antigen-binding fragment thereof is an internalizing antibody or internalizing antigen-binding fragment. In some embodiments, the anti-CD48 antibody comprises a human IgG1 heavy chain constant domain or a modified IgG1 heavy chain constant domain. In some embodiments, the IgG1 heavy chain constant domain comprises a cysteine residue (C) at the amino acid positions corresponding to 152 and 375 in a wild-type (unmodified) IgG1 heavy chain constant domain numbered according to EU numbering system.

In some embodiments, the anti-CD48 antibody comprises the heavy chain amino acid sequence of SEQ ID NO:69 or a sequence that is at least 95% identical to SEQ ID NO:69, and the light chain amino acid sequence of SEQ ID NO:70 or a sequence that is at least 95% identical to SEQ ID NO:70. In some embodiments, the anti-CD48 antibody comprises the heavy chain amino acid sequence of SEQ ID NO:69 and the light chain amino acid sequence of SEQ ID NO:70, or sequences that are at least 95% identical to the disclosed sequences. In some embodiments, the anti-CD48 antibody has a heavy chain amino acid sequence that is at least 96%, at least 97%, at least 98%, or at least 99% identical to SEQ ID NO:69 and a light chain amino acid sequence that is at least 96%, at least 97%, at least 98%, or at least 99% identical to SEQ ID NO:70. In some embodiments, the anti-CD48 antibody is SGN-48A, or an antigen-binding fragment thereof.

In some embodiments, the anti-CD48 antibody comprises the heavy chain amino acid sequence of SEQ ID NO:240 or a sequence that is at least 95% identical to SEQ ID NO:240, and the light chain amino acid sequence of SEQ ID NO:243 or a sequence that is at least 95% identical to SEQ ID NO:243. In some embodiments, the anti-CD48 antibody comprises the heavy chain amino acid sequence of SEQ ID NO:240 and the light chain amino acid sequence of SEQ ID NO:243, or sequences that are at least 95% identical to the disclosed sequences. In some embodiments, the anti-CD48 antibody has a heavy chain amino acid sequence that is at least 96%, at least 97%, at least 98%, or at least 99% identical to SEQ ID NO:240 and a light chain amino acid sequence that is at least 96%, at least 97%, at least 98%, or at least 99% identical to SEQ ID NO:243.

In some embodiments, the anti-CD48 antibody comprises the heavy chain amino acid sequence of SEQ ID NO:242 or a sequence that is at least 95% identical to SEQ ID NO:242, and the light chain amino acid sequence of SEQ ID NO:243 or a sequence that is at least 95% identical to SEQ ID NO:243. In some embodiments, the anti-CD48 antibody comprises the heavy chain amino acid sequence of SEQ ID NO:242 and the light chain amino acid sequence of SEQ ID NO:243, or sequences that are at least 95% identical to the disclosed sequences. In some embodiments, the anti-CD48 antibody has a heavy chain amino acid sequence that is at least 96%, at least 97%, at least 98%, or at least 99% identical to SEQ ID NO:242 and a light chain amino acid sequence that is at least 96%, at least 97%, at least 98%, or at least 99% identical to SEQ ID NO:243.

In some embodiments, the anti-CD48 antibody or antigen-binding fragment thereof comprises the three heavy chain CDRs and three light chain CDRs of SGN-CD48A (MEM/MEM102) or wherein the CDRs include no more than one, two, three, four, five, or six amino acid additions, deletions or substitutions of HCDR1 (SEQ ID NO:51), HCDR2 (SEQ ID NO:52), HCDR3 (SEQ ID NO:53); LCDR1 (SEQ ID NO:54), LCDR2 (SEQ ID NO:55), and LCDR3 (SEQ ID NO:56).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:271), HCDR2 (SEQ ID NO:272), HCDR3 (SEQ ID NO:273); LCDR1 (SEQ ID NO:281), LCDR2 (SEQ ID NO:282), and LCDR3 (SEQ ID NO:283).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:274), HCDR2 (SEQ ID NO:275), HCDR3 (SEQ ID NO:273); LCDR1 (SEQ ID NO:284), LCDR2 (SEQ ID NO:285), and LCDR3 (SEQ ID NO:286).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:276), HCDR2 (SEQ ID NO:277), HCDR3 (SEQ ID NO:278); LCDR1 (SEQ ID NO:287), LCDR2 (SEQ ID NO:282), and LCDR3 (SEQ ID NO:286).

In some embodiments, the anti-CD48 antibody or antigen-binding fragment thereof comprises the three heavy chain CDRs and three light chain CDRs of NY920 or wherein the CDRs include no more than one, two, three, four, five, or six amino acid additions, deletions or substitutions of HCDR1 (SEQ ID NO:279), HCDR2 (SEQ ID NO:275), HCDR3 (SEQ ID NO:273); LCDR1 (SEQ ID NO:284), LCDR2 (SEQ ID NO:288), and LCDR3 (SEQ ID NO:286).

In some embodiments, the target antigen for an ADC is CD74.

In some embodiments, the anti-CD74 antibody comprises the heavy chain amino acid sequence of SEQ ID NO:118 or a sequence that is at least 95% identical to SEQ ID NO:118, and the light chain amino acid sequence of SEQ ID NO:119 or a sequence that is at least 95% identical to SEQ ID NO:119. In some embodiments, the anti-CD74 antibody comprises the heavy chain amino acid sequence of SEQ ID NO:118 and the light chain amino acid sequence of SEQ ID NO:119, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:118 and a light chain amino acid sequence that is at least 96%, at least 97%, at least 98%, or at least 99% identical to SEQ ID NO:119. In some embodiments, the anti-CD74 antibody is milatuzumab, or an antigen-binding fragment thereof.

In some embodiments, the anti-CD74 antibody comprises the heavy chain amino acid sequence of SEQ ID NO: 118 or a sequence that is at least 95% identical to SEQ ID NO:118, and the light chain amino acid sequence of SEQ ID NO:237 or a sequence that is at least 95% identical to SEQ ID NO:237. In some embodiments, the anti-CD74 antibody comprises the heavy chain amino acid sequence of SEQ ID NO:118 and the light chain amino acid sequence of SEQ ID NO:237, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:119 and a light chain amino acid sequence that is at least 96%, at least 97%, at least 98%, or at least 99% identical to SEQ ID NO:234.

In some embodiments, the anti-CD74 antibody comprises the heavy chain amino acid sequence of SEQ ID NO:236 or a sequence that is at least 95% identical to SEQ ID NO:236, and the light chain amino acid sequence of SEQ ID NO:237 or a sequence that is at least 95% identical to SEQ ID NO:237. In some embodiments, the anti-CD74 antibody comprises the heavy chain amino acid sequence of SEQ ID NO:236 and the light chain amino acid sequence of SEQ ID NO:237, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:236 and a light chain amino acid sequence that is at least 96%, at least 97%, at least 98%, or at least 99% identical to SEQ ID NO:237.

In some embodiments, the anti-CD74 antibody comprises the heavy chain amino acid sequence of SEQ ID NO:118 or a sequence that is at least 95% identical to SEQ ID NO:118, and the light chain amino acid sequence of SEQ ID NO:239 or a sequence that is at least 95% identical to SEQ ID NO:239. In some embodiments, the anti-CD74 antibody comprises the heavy chain amino acid sequence of SEQ ID NO:118 and the light chain amino acid sequence of SEQ ID NO:239, or sequences that are at least 95% identical to the disclosed sequences. In some embodiments, the anti-CD74 antibody has a heavy chain amino acid sequence that is at least 96%, at least 97%, at least 98%, or at least 99% identical to SEQ ID NO:118 and a light chain amino acid sequence that is at least 96%, at least 97%, at least 98%, or at least 99% identical to SEQ ID NO:239.

In some embodiments, the anti-CD74 antibody or antigen-binding fragment thereof comprises a heavy chain variable region comprising the amino acid sequence of SEQ ID NO:153, and a light chain variable region comprising the amino acid sequence of SEQ ID NO:262. In some embodiments, the anti-CD74 antibody or antigen-binding fragment thereof comprises the heavy chain variable region amino acid sequence of SEQ ID NO:153 and the light chain variable region amino acid sequence of SEQ ID NO:262, or sequences that are at least 95% identical to the disclosed sequences. In some embodiments, the anti-CD74 antibody or antigen-binding fragment thereof has a heavy chain variable region amino acid sequence that is at least 96%, at least 97%, at least 98%, or at least 99% identical to SEQ ID NO:153 and/or a light chain variable region amino acid sequence that is at least 96%, at least 97%, at least 98%, or at least 99% identical to SEQ ID NO:262.

In some embodiments, the anti-CD74 antibody or antigen-binding fragment thereof comprises a heavy chain variable region comprising the amino acid sequence of SEQ ID NO:153, and a light chain variable region comprising the amino acid sequence of SEQ ID NO:267. In some embodiments, the anti-CD74 antibody or antigen-binding fragment thereof comprises the heavy chain variable region amino acid sequence of SEQ ID NO:153 and the light chain variable region amino acid sequence of SEQ ID NO:267, or sequences that are at least 95% identical to the disclosed sequences. In some embodiments, the anti-CD74 antibody or antigen-binding fragment thereof has a heavy chain variable region amino acid sequence that is at least 96%, at least 97%, at least 98%, or at least 99% identical to SEQ ID NO:153 and/or a light chain variable region amino acid sequence that is at least 96%, at least 97%, at least 98%, or at least 99% identical to SEQ ID NO:267.

In some embodiments, the anti-CD74 antibody or antigen-binding fragment thereof comprises three heavy chain CDRs and three light chain CDRs as follows: heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:263, light chain CDR2 (LCDR2) consisting of SEQ ID NO:264, and light chain CDR3 (LCDR3) consisting of SEQ ID NO:265.

In some embodiments, the anti-CD74 antibody or antigen-binding fragment thereof comprises three heavy chain CDRs and three light chain CDRs as follows: heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:215, light chain CDR2 (LCDR2) consisting of SEQ ID NO:264, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:266, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:256, heavy chain CDR2 (HCDR2) consisting of SEQ ID NO:257, heavy chain CDR3 (HCDR3) consisting of SEQ ID NO:171; light chain CDR1 (LCDR1) consisting of SEQ ID NO:268, light chain CDR2 (LCDR2) consisting of SEQ ID NO:264, and light chain CDR3 (LCDR3) consisting of SEQ ID NO:265.

In some embodiments, the anti-CD74 antibody or antigen-binding fragment thereof comprises three heavy chain CDRs and three light chain CDRs as follows: heavy chain CDR1 (HCDR1) consisting of SEQ ID NO:258, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:259, heavy chain CDR2 (HCDR2) consisting of SEQ ID NO:260, heavy chain CDR3 (HCDR3) consisting of SEQ ID NO:261; light chain CDR1 (LCDR1) consisting of SEQ ID NO:269, light chain CDR2 (LCDR2) consisting of SEQ ID NO:264, and light chain CDR3 (LCDR3) consisting of SEQ ID NO:174.

In some embodiments, the anti-CD74 antibody or antigen-binding fragment thereof comprises three heavy chain CDRs and three light chain CDRs as follows: heavy chain CDR1 (HCDR1) consisting of SEQ ID NO:169, heavy chain CDR2 (HCDR2) consisting of SEQ ID NO:170, heavy chain CDR3 (HCDR3) consisting of SEQ ID NO:171; light chain CDR1 (LCDR1) consisting of SEQ ID NO:172, light chain CDR2 (LCDR2) consisting of SEQ ID NO:173, and light chain CDR3 (LCDR3) consisting of SEQ ID NO:174.

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:256), HCDR2 (SEQ ID NO:257), HCDR3 (SEQ ID NO:171); LCDR1 (SEQ ID NO:263), LCDR2 (SEQ ID NO:264), and LCDR3 (SEQ ID NO:265).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:258), HCDR2 (SEQ ID NO:170), HCDR3 (SEQ ID NO:171); LCDR1 (SEQ ID NO:266), LCDR2 (SEQ ID NO:173), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:259), HCDR2 (SEQ ID NO:260), HCDR3 (SEQ ID NO:261); LCDR1 (SEQ ID NO:267), LCDR2 (SEQ ID NO:264), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:169), HCDR2 (SEQ ID NO:170), HCDR3 (SEQ ID NO:171); LCDR1 (SEQ ID NO:266), LCDR2 (SEQ ID NO:173), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:256), HCDR2 (SEQ ID NO:257), HCDR3 (SEQ ID NO:171); LCDR1 (SEQ ID NO:268), LCDR2 (SEQ ID NO:264), and LCDR3 (SEQ ID NO:265).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:258), HCDR2 (SEQ ID NO:170), HCDR3 (SEQ ID NO:171); LCDR1 (SEQ ID NO:172), LCDR2 (SEQ ID NO:173), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:259), HCDR2 (SEQ ID NO:260), HCDR3 (SEQ ID NO:261); LCDR1 (SEQ ID NO:269), LCDR2 (SEQ ID NO:264), and LCDR3 (SEQ ID NO:174).

In some embodiments, the anti-CD74 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:169), HCDR2 (SEQ ID NO:170), HCDR3 (SEQ ID NO:171); LCDR1 (SEQ ID NO:172), LCDR2 (SEQ ID NO:173), and LCDR3 (SEQ ID NO:174).

In some embodiments, the target antigen for an ADC is EphA2.

In some embodiments, the anti-EphA2 antibody or antigen-binding fragment thereof comprises a heavy chain variable region comprising the amino acid sequence of SEQ ID NO:318, and a light chain variable region comprising the amino acid sequence of SEQ ID NO:329. In some embodiments, the anti-EphA2 antibody or antigen-binding fragment thereof comprises the heavy chain variable region amino acid sequence of SEQ ID NO:318 and the light chain variable region amino acid sequence of SEQ ID NO:329, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO:318 and/or a light chain variable region amino acid sequence that is at least 96%, at least 97%, at least 98%, or at least 99% identical to SEQ ID NO:329.

In some embodiments, the anti-EphA2 antibody comprises the heavy chain amino acid sequence of SEQ ID NO:252 or a sequence that is at least 95% identical to SEQ ID NO:252, and the light chain amino acid sequence of SEQ ID NO:254 or a sequence that is at least 95% identical to SEQ ID NO:254. In some embodiments, the anti-EphA2 antibody comprises the heavy chain amino acid sequence of SEQ ID NO:252 and the light chain amino acid sequence of SEQ ID NO:254, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO:252 and a light chain amino acid sequence that is at least 96%, at least 97%, at least 98%, or at least 99% identical to SEQ ID NO:254.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:319, heavy chain CDR2 (HCDR2) consisting of SEQ ID NO:320, heavy chain CDR3 (HCDR3) consisting of SEQ ID NO:321; light chain CDR1 (LCDR1) consisting of SEQ ID NO:330, light chain CDR2 (LCDR2) consisting of SEQ ID NO:331, and light chain CDR3 (LCDR3) consisting of SEQ ID NO:332.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:322, heavy chain CDR2 (HCDR2) consisting of SEQ ID NO:323, heavy chain CDR3 (HCDR3) consisting of SEQ ID NO:324; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:325, heavy chain CDR2 (HCDR2) consisting of SEQ ID NO:326, heavy chain CDR3 (HCDR3) consisting of SEQ ID NO:327; light chain CDR1 (LCDR1) consisting of SEQ ID NO:336, light chain CDR2 (LCDR2) consisting of SEQ ID NO:331, and light chain CDR3 (LCDR3) consisting of SEQ ID NO:335.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:328, heavy chain CDR2 (HCDR2) consisting of SEQ ID NO:323, heavy chain CDR3 (HCDR3) consisting of SEQ ID NO:321; light chain CDR1 (LCDR1) consisting of SEQ ID NO:333, light chain CDR2 (LCDR2) consisting of SEQ ID NO:334, and light chain CDR3 (LCDR3) consisting of SEQ ID NO:335.

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:319), HCDR2 (SEQ ID NO:320), HCDR3 (SEQ ID NO:321); LCDR1 (SEQ ID NO:330), LCDR2 (SEQ ID NO:331), and LCDR3 (SEQ ID NO:332).

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:322), HCDR2 (SEQ ID NO:323), HCDR3 (SEQ ID NO:324); LCDR1 (SEQ ID NO:333), LCDR2 (SEQ ID NO:334), and LCDR3 (SEQ ID NO:335).

In some embodiments, the anti-EphA2 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:325), HCDR2 (SEQ ID NO:326), HCDR3 (SEQ ID NO:327); LCDR1 (SEQ ID NO:336), LCDR2 (SEQ ID NO:331), and LCDR3 (SEQ ID NO:335).

In some embodiments, the anti-EphA2 antibody or antigen-binding fragment thereof comprises the three heavy chain CDRs and three light chain CDRs of milatuzumab or wherein the CDRs include no more than one, two, three, four, five, or six amino acid additions, deletions or substitutions of HCDR1 (SEQ ID NO:328), HCDR2 (SEQ ID NO:323), HCDR3 (SEQ ID NO:321); LCDR1 (SEQ ID NO:333), LCDR2 (SEQ ID NO:334), and LCDR3 (SEQ ID NO:335).

Residues in two or more polypeptides are said to “correspond” if the residues occupy an analogous position in the polypeptide structures. Analogous positions in two or more polypeptides can be determined by aligning the polypeptide sequences based on amino acid sequence or structural similarities. Those skilled in the art understand that it may be necessary to introduce gaps in either sequence to produce a satisfactory alignment.

In some embodiments, amino acid substitutions are of single residues. Insertions usually will be on the order of from about 1 to about 20 amino acid residues, although considerably larger insertions may be tolerated as long as biological function is retained (e.g., binding to a target antigen). Deletions usually range from about 1 to about 20 amino acid residues, although in some cases deletions may be much larger. Substitutions, deletions, insertions, or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances. Conservative substitutions can be made in accordance with the following chart depicted as Table E.

TABLE E

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In some embodiments where variant antibody sequences are used in an ADC, the variants typically exhibit the same qualitative biological activity and will elicit the same immune response, although variants may also be selected to modify the characteristics of the antigen binding proteins as needed. Alternatively, the variant may be designed such that the biological activity of the antigen binding protein is altered. For example, glycosylation sites may be altered or removed.

Various antibodies may be used with the ADCs used herein to target cancer cells. As shown below, the linker-payloads in the ADCs disclosed herein are surprisingly effective with different tumor antigen-targeting antibodies. Suitable antigens expressed on cancer cells but not healthy cells, or expressed on cancer cells at a higher level than on healthy cells, are known in the art, as are antibodies directed against them. Further antibodies against those antigen targets may be prepared by those of skill in the art. These antibodies may be used with the linkers disclosed herein. In some embodiments, the antibody or antigen-binding fragment targets PCAD, and in some embodiments the PCAD-targeting antibody or antigen-binding fragment is NOV169N31Q. In some embodiments, the antibody or antigen-binding fragment targets PCAD, and in some embodiments the PCAD-targeting antibody or antigen-binding fragment is CQY679. In some embodiments, the antibody or antigen-binding fragment targets EPhA2, and in some embodiments the EphA2-targeting antibody or antigen-binding fragment is EphA2 1C1. In some embodiments, the antibody or antigen-binding fragment targets CD74, and in some embodiments the CD74-targeting antibody or antigen-binding fragment is milatuzumab. In some embodiments, the antibody or antigen-binding fragment targets CD74, and in some embodiments the CD74-targeting antibody or antigen-binding fragment is VHmil×VK1aNQ. In some embodiments, the antibody or antigen-binding fragment targets CD48, and in some embodiments the CD48-targeting antibody or antigen-binding fragment is SGN-CD48A (MEM/MEM102). In some embodiments, the antibody or antigen-binding fragment targets CD48, and in some embodiments the CD48-targeting antibody or antigen-binding fragment is CD48 NY920. In some embodiments, the antibody or antigen-binding fragment targets HER2, and in some embodiments the HER2-targeting antibody or antigen-binding fragment is trastuzumab, and in some embodiments the HER2-targeting antibody or antigen-binding fragment is disitamab. In some embodiments, the antibody or antigen-binding fragment targets TROP2, and in some embodiments the TROP2-targeting antibody or antigen-binding fragment is datopotamab. In some embodiments, the antibody or antigen-binding fragment targets B7-H3, and in some embodiments the B7-H3-targeting antibody or antigen-binding fragment is ABBV-155, and in some embodiments the B7-H3-targeting antibody or antigen-binding fragment is DS-5573a. In some embodiments, the antibody or antigen-binding fragment targets 5T4. In some embodiments, while the disclosed linkers are surprisingly effective with several different tumor —targeting antibodies, PCAD-targeting antibodies such as NOV169N31Q or CQY679, EphA2-targeting antibodies such as EphA2 1C1, CD48-targeting antibodies such as CD48 NY920 or SGN-CD48A (MEM/MEM102), CD74-targeting antibodies such as milatuzumab or VHmil×VK1aNQ, HER2-targeting antibodies such as trastuzumab or disitamab, TROP2-targeting antibodies such as datopotamab, B7-H3-targeting antibodies such as ABBV-155 or DS-5573a, and 5T4-targeting antibodies, provided particularly improved drug:antibody ratio, aggregation level, stability (i.e., in vitro and in vivo stability), tumor targeting (i.e., cytotoxicity, potency), minimized off-target killing, and/or treatment efficacy. Improved treatment efficacy can be measured in vitro or in vivo, and may include reduced tumor growth rate and/or reduced tumor volume.

In some embodiments, alternate antibodies to the same targets or antibodies to different antigen targets are used and provide at least some of the favorable functional properties described above (e.g., improved stability, improved tumor targeting, improved treatment efficacy, etc.). In some embodiments, some or all of these favorable functional properties are observed when the disclosed linkers and payloads are conjugated to an alternate HER2, CD74, CD48, EphA2 or PCAD-targeting antibody or antigen-binding fragment. In other embodiments, some or all of these favorable functional properties are observed when the disclosed linkers and payloads are conjugated to an HER2-targeting antibody or antigen-binding fragment. In some embodiments, the antibody or antigen-binding fragment targets HER2. In some embodiments, the HER2-targeting antibody or antigen-binding fragment is trastuzumab. In other embodiments, some or all of these favorable functional properties are observed when the disclosed linkers and drugs are conjugated to an CD74-targeting antibody or antigen-binding fragment. In some embodiments, the antibody or antigen-binding fragment targets CD74. In some embodiments, the CD74-targeting antibody or antigen-binding fragment is milatuzumab. In some embodiments, the CD74-targeting antibody or antigen-binding fragment is VHmil×VK1aNQ. In other embodiments, some or all of these favorable functional properties are observed when the disclosed linkers and drugs are conjugated to an CD48-targeting antibody or antigen-binding fragment. In some embodiments, the antibody or antigen-binding fragment targets CD48. In some embodiments, the CD48-targeting antibody or antigen-binding fragment is CD48 NY920 or SGN-CD48A (MEM/MEM102). In other embodiments, some or all of these favorable functional properties are observed when the disclosed linkers and drugs are conjugated to an EphA2-targeting antibody or antigen-binding fragment. In some embodiments, the antibody or antigen-binding fragment targets EphA2. In some embodiments, the EphA2-targeting antibody or antigen-binding fragment is anti-EphA2 1C1. In other embodiments, some or all of these favorable functional properties are observed when the disclosed linkers and drugs are conjugated to an PCAD-targeting antibody or antigen-binding fragment. In some embodiments, the antibody or antigen-binding fragment targets PCAD. In some embodiments, the PCAD-targeting antibody or antigen-binding fragment is NOV169N31Q or CQY679. In other embodiments, some or all of these favorable functional properties are observed when the disclosed linkers and drugs, are conjugated to an TROP2-targeting antibody or antigen-binding fragment. In some embodiments, the antibody or antigen-binding fragment targets TROP2. In some embodiments, the TROP2-targeting antibody or antigen-binding fragment is datopotamab. In other embodiments, some or all of these favorable functional properties are observed when the disclosed linkers and drugs are conjugated to an B7-H3-targeting antibody or antigen-binding fragment. In some embodiments, the antibody or antigen-binding fragment targets B7-H3. In some embodiments, the B7-H3-targeting antibody or antigen-binding fragment is ABBV-155 or DS-5573a. In other embodiments, some or all of these favorable functional properties are observed when the disclosed linkers and drugs are conjugated to an 5T4-targeting antibody or antigen-binding fragment. In some embodiments, the antibody or antigen-binding fragment targets 5T4.

### Linkers

In some embodiments, the linker in an ADC is stable extracellularly in a sufficient manner to be therapeutically effective. In some embodiments, the linker is stable outside a cell, such that the ADC remains intact when present in extracellular conditions (e.g., prior to transport or delivery into a cell). The term “intact,” used in the context of an ADC, means that the antibody or antigen-binding fragment remains attached to the drug moiety.

As used herein, “stable,” in the context of a linker or ADC comprising a linker, means that no more than 20%, no more than about 15%, no more than about 10%, no more than about 5%, no more than about 3%, or no more than about 1% of the linkers (or any percentage in between) in a sample of ADC are cleaved (or in the case of an overall ADC are otherwise not intact) when the ADC is present in extracellular conditions. In some embodiments, the linkers and/or ADCs disclosed herein are stable compared to alternate linkers and/or ADCs with alternate linkers. In some embodiments, the ADCs disclosed herein can remain intact for more than about 48 hours, more than 60 hours, more than about 72 hours, more than about 84 hours, or more than about 96 hours.

Whether a linker is stable extracellularly can be determined, for example, by including an ADC in plasma for a predetermined time period (e.g., 2, 4, 6, 8, 16, 24, 48, or 72 hours) and then quantifying the amount of free drug moiety present in the plasma. Stability may allow the ADC time to localize to target cancer cells and prevent the premature release of the drug moiety, which could lower the therapeutic index of the ADC by indiscriminately damaging both normal and cancer tissues. In some embodiments, the linker is stable outside of a target cell and releases the drug moiety from the ADC once inside of the cell, such that the drug can bind to its target. Thus, an effective linker will: (i) maintain the specific binding properties of the antibody or antigen-binding fragment; (ii) allow delivery, e.g., intracellular delivery, of the drug moiety via stable attachment to the antibody or antigen-binding fragment; (iii) remain stable and intact until the ADC has been transported or delivered to its target site; and (iv) allow for the therapeutic effect, e.g., cytotoxic effect, of the drug moiety after cleavage or alternate release mechanism.

Linkers may impact the physico-chemical properties of an ADC. As many cytotoxic agents are hydrophobic in nature, linking them to the antibody with an additional hydrophobic moiety may lead to aggregation. ADC aggregates are insoluble and often limit achievable drug loading onto the antibody, which can negatively affect the potency of the ADC. Protein aggregates of biologics, in general, have also been linked to increased immunogenicity. As shown below, linkers disclosed herein result in ADCs with low aggregation levels and desirable levels of drug loading.

A linker may be “cleavable” or “non-cleavable” (Ducry and Stump (2010) Bioconjugate Chem. 21:5-13). Cleavable linkers are designed to release the drug moiety when subjected to certain environment factors, e.g., when internalized into the target cell, whereas non-cleavable linkers generally rely on the degradation of the antibody or antigen-binding fragment itself.

The term “alkyl”, as used herein, refers to a straight or branched hydrocarbon chain radical consisting solely of carbon and hydrogen atoms, containing no unsaturation. The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl (a C₁alkyl), ethyl (a C₂alkyl), 1-methylethyl (a C₃alkyl), n-propyl (a C₃alkyl), isopropyl (a C₃alkyl), n-butyl (a C₄alkyl), isobutyl (a C₄alkyl), sec-butyl (a C₄alkyl), tert-butyl (a C₄alkyl), n-pentyl (a C₅alkyl), isopentyl (a C₅alkyl), neopentyl (a C₅alkyl) and hexyl (a C₆alkyl).

The term “alkenyl”, as used herein, refers to a straight or branched hydrocarbon chain radical group consisting solely of carbon and hydrogen atoms, containing at least one double bond. The term “C₂-C₆alkenyl”, as used herein, refers to a straight or branched hydrocarbon chain radical group consisting solely of carbon and hydrogen atoms, containing at least one double bond, having from two to six carbon atoms, which is attached to the rest of the molecule by a single bond. Non-limiting examples of “C₂-C₆alkenyl” groups include ethenyl (a C₂alkenyl), prop-1-enyl (a C₃alkenyl), but-1-enyl (a C₄alkenyl), pent-1-enyl (a C₅alkenyl), pent-4-enyl (a C₅alkenyl), penta-1,4-dienyl (a C₅alkenyl), hexa-1-enyl (a C₆alkenyl), hexa-2-enyl (a C₆alkenyl), hexa-3-enyl (a C₆alkenyl), hexa-1-,4-dienyl (a C₆alkenyl), hexa-1-,5-dienyl (a C₆alkenyl) and hexa-2-,4-dienyl (a C₆alkenyl). The term “C₂-C₃alkenyl”, as used herein, refers to a straight or branched hydrocarbon chain radical group consisting solely of carbon and hydrogen atoms, containing at least one double bond, having from two to three carbon atoms, which is attached to the rest of the molecule by a single bond. Non-limiting examples of “C₂-C₃alkenyl” groups include ethenyl (a C₂alkenyl) and prop-1-enyl (a C₃alkenyl).

The term “alkylene”, as used herein, refers to a bivalent straight or branched hydrocarbon chain radical consisting solely of carbon and hydrogen atoms and containing no unsaturation.

The term “C₁-C₆alkylene”, as used herein, refers to a bivalent straight or branched hydrocarbon chain radical consisting solely of carbon and hydrogen atoms, containing no unsaturation, having from one to six carbon atoms. Non-limiting examples of “C₁-C₆alkylene” groups include methylene (a C₁alkylene), ethylene (a C₂alkylene), 1-methylethylene (a C₃alkylene), n-propylene (a C₃alkylene), isopropylene (a C₃alkylene), n-butylene (a C₄alkylene), isobutylene (a C₄alkylene), sec-butylene (a C₄alkylene), tert-butylene (a C₄alkylene), n-pentylene (a C₅alkylene), isopentylene (a C₅alkylene), neopentylene (a C₅alkylene), and hexylene (a C₆alkylene).

The term “alkenylene”, as used herein, refers to a bivalent straight or branched hydrocarbon chain radical consisting solely of carbon and hydrogen atoms and containing at least one double bond. The term “C₂-C₆alkenylene”, as used herein, refers to a bivalent straight or branched hydrocarbon chain radical group consisting solely of carbon and hydrogen atoms, containing at least one double bond, and having from two to six carbon atoms. Non-limiting examples of “C₂-C₆alkenylene” groups include ethenylene (a C₂alkenylene), prop-1-enylene (a C₃alkenylene), but-1-enylene (a C₄alkenylene), pent-1-enylene (a C₅alkenylene), pent-4-enylene (a C₅alkenylene), penta-1,4-dienylene (a C₅alkenylene), hexa-1-enylene (a C₆alkenylene), hexa-2-enylene (a C₆alkenylene), hexa-3-enylene (a C6alkenylene), hexa-1-,4-dienylene (a C₆alkenylene), hexa-1-,5-dienylene (a C₆alkenylene) and hexa-2-,4-dienylene (a C₆alkenylene).

The term “C₂-C₆alkenylene”, as used herein, refers to a bivalent straight or branched hydrocarbon chain radical group consisting solely of carbon and hydrogen atoms, containing at least one double bond, and having from two to three carbon atoms. Non-limiting examples of “C₂—C₃alkenylene” groups include ethenylene (a C₂alkenylene) and prop-1-enylene (a C₃alkenylene).

The term “aryl” as used herein, refers to a phenyl, naphthyl, biphenyl or indenyl group.

The term “heteroaryl” as used herein, refers any mono- or bi-cyclic group composed of from 5 to 10 ring members, having at least one aromatic moiety and containing from 1 to 4 hetero atoms selected from oxygen, sulphur and nitrogen (including quaternary nitrogens).

The term “cycloalkyl” as used herein, refers to any mono- or bi-cyclic non-aromatic carbocyclic group containing from 3 to 10 ring members, which may include fused, bridged or spiro ring systems. Non-limiting examples of fused bicyclic or bridged ring systems include bicyclo[1.1.1]pentane, bicyclo[2.1.1]hexane, bicyclo[2.2.1]heptane, bicyclo[3.1.1]heptane, bicyclo[3.2.1]octane, and bicyclo[2.2.2]octane. Non-limiting examples monocyclic C₃-C₈cycloalkyl groups include cyclopropyl, cyclobutyl, cyclopentyl, cyclohexyl, cycloheptyl and cyclooctyl groups. The term “heterocycloalkyl” means any mono- or bi-cyclic non-aromatic carbocyclic group, composed of from 3 to 10 ring members, and containing from one to 3 hetero atoms selected from oxygen, sulphur, SO, SO₂ and nitrogen, it being understood that bicyclic group may be fused or spiro type. C₃-C₈heterocycloalkyl refers to heterocycloalkyl having 3 to 8 ring carbon atoms. The heterocycloalkyl can have 4 to 10 ring members.

The term heteroarylene, cycloalkylene, heterocycloalkylene mean a divalent heteroaryl, cycloalkyl and heterocycloalkyl.

The term “haloalkyl,” as used herein, refers to a linear or branched alkyl chain substituted with one or more halogen groups in place of hydrogens along the hydrocarbon chain. Examples of halogen groups suitable for substitution in the haloalkyl group include Fluorine, Bromine, Chlorine, and Iodine. Haloalkyl groups may include substitution with multiple halogen groups in place of hydrogens in an alkyl chain, wherein said halogen groups can be attached to the same carbon or to another carbon in the alkyl chain.

As used herein, the alkyl, alkenyl, alkynyl, alkoxy, amino, aryl, heteroaryl, cycloalkyl, and heterocycloalkyl groups may be optionally substituted by 1 to 4 groups selected from optionally substituted linear or branched (C₁-C₆)alkyl, optionally substituted linear or branched (C2-C₆)alkenyl group, optionally substituted linear or branched (C₂-C₆)alkynyl group, optionally substituted linear or branched (C₁-C₆)alkoxy, optionally substituted (C₁-C₆)alkyl-S—, hydroxy, oxo (or N-oxide where appropriate), nitro, cyano, —C(O)—OR₀′, —O—C(O)—R₀′, —C(O)—NR₀′R₀″, —NR₀′R₀″, —(C═NR₀′)—OR₀″, linear or branched (C₁-C₆) haloalkyl, trifluoromethoxy, or halogen, wherein R₀′ and R₀″ are each independently a hydrogen atom or an optionally substituted linear or branched (C₁-C₆)alkyl group, and wherein one or more of the carbon atoms of linear or branched (C₁-C₆)alkyl group is optionally deuterated.

The term “polyoxyethylene”, “polyethylene glycol” or “PEG”, as used herein, refers to a linear chain, a branched chain or a star shaped configuration comprised of (OCH₂CH₂) groups. In certain embodiments a polyethylene or PEG group is —(OCH₂CH₂)ₜ*—, where t is 1-40 or 4-40, and where the “—” indicates the end directed toward the self-immolative spacer and the “*—” indicates the point of attachment to a terminal end group R′ where R″ is OH, OCH₃ or OCH₂CH₂C(═O)OH. In other embodiments a polyethylene or PEG group is —(CH₂CH₂O)ₜ*—, where t is 1-40 or 4-40, and where the “—” indicates the end directed toward the self-immolative spacer and the “*—” indicates the point of attachment to a terminal end group R″ where R^{w} is H, CH₃ or CH₂CH₂C(═O)OH. For example, the term “PEG12” as used herein means that t is 12.

The term “polyalkylene glycol”, as used herein, refers to a linear chain, a branched chain or a star shaped configuration comprised of (O(CH₂)ₘ)ₙ groups. In certain embodiments a polyethylene or PEG group is —(O(CH₂)ₘ)ₜ*—, where m is 1-10, t is 1-40 or 4-40, and where the “-” indicates the end directed toward the self-immolative spacer and the “*—” indicates the point of attachment to a terminal end group R‘ where R’ is OH, OCH₃ or OCH₂CH₂C(═O)OH. In other embodiments a polyethylene or PEG group is —((CH₂)ₘO)ₜ*—, where m is 1-10, t is 1-40 or 4-40, and where the “—” indicates the end directed toward the self-immolative spacer and the “*—” indicates the point of attachment to a terminal end group R″ where R^{w} is H, CH₃ or CH₂CH₂C(═O)OH.

The term “reactive group”, as used herein, is a functional group capable of forming a covalent bond with a functional group of an antibody, an antibody fragment, or another reactive group attached to an antibody or antibody fragment. Non limiting examples of such functional groups include reactive groups of Table 8 provided herein.

The term “attachment group” or “coupling group”, as used herein, refers to a bivalent moiety which links the bridging spacer to the antibody or fragment thereof. The attachment or coupling group is a bivalent moiety formed by the reaction between a reaction group and a functional group on the antibody or fragment thereof. Non limiting examples of such bivalent moieties include the bivalent chemical moieties given in Table F and Table G provided herein.

The term “attachment group” or “coupling group”, as used herein, refers to a bivalent moiety which links the bridging spacer to the antibody or fragment thereof. The attachment or coupling group is a bivalent moiety formed by the reaction between a reaction group and a functional group on the antibody or fragment thereof. Non limiting examples of such bivalent moieties include the bivalent chemical moieties given in Table F and Table G provided herein.

The term “attachment point”, as used herein, refers to a location on the linker that is connected to an antibody or drug. In some embodiments, the location is an atom, such as carbon, nitrogen, sulfur, or oxygen, where the linker connects with an antibody or drug through a covalent bond.

The term “bridging spacer”, as used herein, refers to one or more linker components which are covalently attached together to form a bivalent moiety which links the branching moiety W to the attachment group. Non-limiting examples of the bridging spacer include groups L1-1, L1-2, L1-3, L1-4, L1-5, and L1-6 described herein.

The term “branching moiety”, as used herein, refers to a chemical moiety that connects with three or more groups in the dual linker of the present disclosure. In some embodiment, the branching moiety is N or CR_{w}; wherein R_{w} is H or C₁₋₆alkyl.

The term “cleavable group”, as used herein, refers to a a moiety that can be unstable in vivo. In some embodiments, the “cleavable group” allows for activation of the drug payloads by cleaving the drug from the rest of the conjugate. Operatively defined, the linker is preferably cleaved in vivo by the biological environment. The cleavage may come from any process without limitation, e.g., enzymatic, reductive, pH, etc. In one embodiment, the cleavable group is selected so that activation occurs at the desired site of action, which can be a site in or near the target cells (e.g., carcinoma cells) or tissues such as at the site of therapeutic action or drug activity. Such cleavage may be enzymatic and exemplary enzymatically cleavable groups include natural amino acids or peptide sequences that end with a natural amino acid, and are attached at their carboxyl terminus to the linker. In one embodiment, a cleavable group comprises a pyrophosphate group, a phosphate group, a sugar (e.g. glucuronide) group, a peptide group, and/or a self-immolative group.

The term “enzyme cleavage element”, as used herein, comprises an element that is susceptible to enzymatic cleavage. Nonlimiting examples of the enzymatic cleavage include peptidase-induced cleavage, esterase-induced cleavage, glycosidase induced cleavage, phosphodiesterase induced cleavage, phosphatase induced cleavage, protease induced cleavage, or lipase induced cleavage. In some embodiments, the enzyme cleavage element in the present disclosure refers to a dipeptide group that can be cleaved by a peptidase. In some embodiments, the dipeptide group is selected from a group consisting of E1-1 and E1-2 described herein. In some embodiments, the enzyme cleavage element in the present disclosure comprises a sugar moiety that can be cleaved by a glucosidase, such as a glucuronide group. In some embodiments, the enzyme cleavage element in the present disclosure comprises a phosphate or pyrophosphate moiety that can be cleaved by phosphatases.

In some embodiments, the enzyme cleavage element is represented by

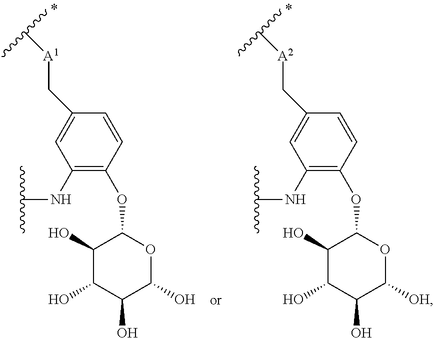

wherein A¹ and A² are as defined herein, [symbol] * [symbol] indicates the point of attachment to E¹ or E²; and indicates the point of attachment to D¹ or D2.

The term “connecting spacer”, as used herein, refers to one or more linker components which are covalently attached together to form a bivalent moiety which links the branching moiety W to the function moiety E¹ or E² which comprises an enzyme cleavage element or a hydrophilic moiety. Nonlimiting examples of the connecting spacer include groups L2-1 through L2-30 described herein.

The term “hydrophilic group”, as used herein, refers to the group that has hydrophilic properties which increases the aqueous solubility of the dual linker is attached to the linker group of the present disclosure. Examples of such hydrophilic groups include, but are not limited to, polyethylene glycols, polyalkylene glycols, sugars, oligosaccharides, polypeptides, a C₂-C₆alkyl substituted with 1 to 3

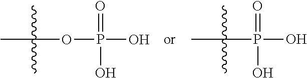 [SMILES: *OP(=O)(O)O ; *P(=O)(O)O]

OH or OH groups, or C₂-C₆alkyl substituted with 1 to 2 substituents independently selected from —OC(═O)NHS(O)₂NHCH₂CH₂OCH₃, —NHC(═O)C₁₋₄alkylene-P(O)(OCH₂CH₃)₂ and —COOH groups.

The term “hydrophilic moiety”, as used herein, refers to the moiety that comprises a functional group having a hydrophilic group attached thereto. In some embodiments, the functional group mentioned here refers to the bivalent peptide spacer described in the present disclosure.

The term “spacer moiety”, as used herein, refers to one or more linker components which are covalently attached together to form a moiety which links the self-immolative group to the hydrophilic moiety or an enzyme cleavage element. In some embodiments, the term “spacer moiety”, as used herein, refers to L⁴ or L⁵ as defined herein. The term “bivalent peptide spacer”, as used herein, refers to bivalent linker comprising one or more amino acid residues covalently attached together to form a moiety which links the bridging spacer to the self immolative spacer or enzyme cleavage element. The one or more amino acid residues can be a residue of amino acids selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine.

In certain embodiments a “bivalent peptide spacer” is a combination of 2 to four amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example -ValCit*; -CitVal*; -AlaAla*; -AlaCit*; -CitAla*; -AsnCit*; -CitAsn*; -CitCit*; -ValGlu*; -GluVal*; -SerCit*; -CitSer*; -LysCit*; -CitLys*; -AspCit*; -CitAsp*; -AlaVal*; -ValAla*; -PheAla*; -AlaPhe*; -PheLys*; -LysPhe*; -ValLys*; -LysVal*; -AlaLys*; -LysAla*; -PheCit*; -CitPhe*; -LeuCit*; -CitLeu*; -IleCit*; -Citlle*; -PheArg*; -ArgPhe*; -CitTrp*; -TrpCit*; -PhePheLys*; -LysPhePhe*; -DPhePheLys*; -DLysPhePhe*; -GlyPheLys*; -LysPheGly*; -GlyPheLeuGly- [SEQ ID NO:145]; -GlyLeuPheGly-[SEQ ID NO:146]; -AlaLeuAlaLeu- [SEQ ID NO:147], -GlyGlyGly*; -GlyGlyGlyGly-[SEQ ID NO:148]; -GlyPheValGly- [SEQ ID NO:149]; and -GlyValPheGly- [SEQ ID NO:150], where the “-” indicates the point of attachment to the bridging spacer and the “*” indicates the point of attachment to the self-immolative spacer.

The term “linker component”, as used herein, refers to a chemical moiety that is a part of the linker. Examples of linker components include: an alkylene group: —(CH₂)ₙ— which can either be linear or branched (where in this instance n is 1-18); an alkenylene group; an alkynylene group; an alkenyl group; an alkynyl group; an ethylene glycol unit: —OCH₂CH₂—or —CH₂CH₂O—; an polyethylene glycol unit: (—CH₂CH₂O—)x (where x in this instance is 2-20); —O—; —S—; a carbonyl: —C(═O); an ester: C(═O)—O or O—C(═O); a carbonate: —OC(═O)O—; an amine: —NH—; an tertiary amine; an amide: —C(═O)—NH—, —NH—C(═O)— or —C(═O)N(C₁₋₆alkyl); a carbamate: —OC(═O)NH—or —NHC(═O)O; a urea: —NHC(═O)NH; a sulfonamide: —S(O)₂NH—or —NHS(O)₂; an ether: —CH₂O—or —OCH₂; an alkylene substituted with one or more groups independently selected from carboxy, sulfonate, hydroxyl, amine, amino acid, saccharide, phosphate and phosphonate); an alkenylene substituted with one or more groups independently selected from carboxy, sulfonate, hydroxyl, amine, amino acid, saccharide, phosphate and phosphonate); an alkynylene substituted with one or more groups independently selected from carboxy, sulfonate, hydroxyl, amine, amino acid, saccharide, phosphate and phosphonate); a C₁-C₁₀alkylene in which one or more methylene groups is replace by one or more —S—, —NH— or —O—moieties; a ring systems having two available points of attachment such as a divalent ring selected from phenyl (including 1,2-1,3- and 1,4-di-substituted phenyls), a C₅-C₆ heteroaryl, a C₃-C₈ cycloalkyl (including 1,1-disubstituted cyclopropyl, cyclobutyl, cyclopentyl or cyclohexyl, and 1,4-disubstituted cyclohexyl), and a C₄-C₈ heterocycloalkyl; a residue of an amino acid selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine; a combination of 2 or more amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit; and a self-immolative spacer, wherein the self-immolative spacer comprises one or more protecting (triggering) groups which are susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

In addition, a linker component can be a chemical moiety which is readily formed by reaction between two reactive groups. Non-limiting examples of such chemical moieties are given in Table F.

TABLE F

| Reactive Group | Reactive Group | |
|---|---|---|
| 1 | 2 | |
| (RG1) | (RG2) | Chemical Moiety |
| a thiol | a thiol | —S—S— |
| a thiol | a maleimide | 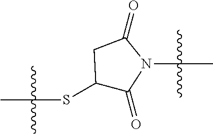 [SMILES: *SC1CC(=O)N(*)C1=O] |
| a thiol | a haloacetamide | 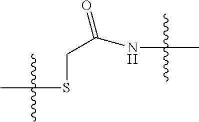 [SMILES: *NC(=O)CS*] |
| an azide | an alkyne | 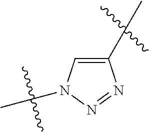 [SMILES: *c1cn(*)nn1] |
| | | or |
| | | 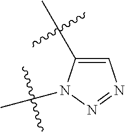 [SMILES: *c1cnnn1*] |
| an azide | a triaryl phosphine | 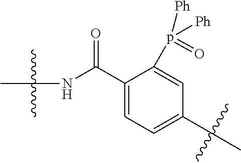 |
| an azide | a cyclooctyne | 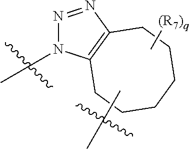 |
| | | or |
| | | 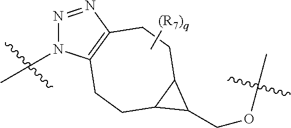 |
| | | or |
| | | 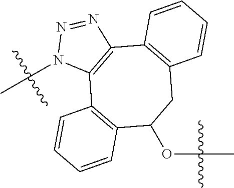 [SMILES: *OC1Cc2ccccc2-c2nnn(*)c2-c2ccccc21] |
| an azide | an oxanobornadiene | 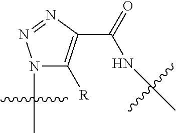 |
| a triaryl phosphine | an azide | 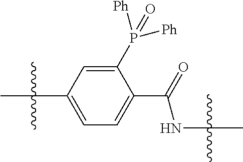 [SMILES: *NC(=O)c1ccc(*)cc1P(=O)(c1ccccc1)c1ccccc1] |
| an oxanobornadiene | an azide | 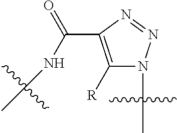 |
| an alkyne | an azide | 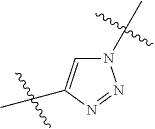 [SMILES: *c1cn(*)nn1] |
| | | or |
| | | 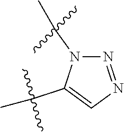 [SMILES: *c1cnnn1*] |
| a cyclooctyne | azide | 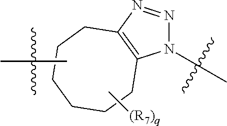 |
| | | or |
| | | 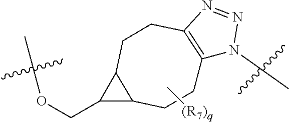 |
| | | or |
| | | 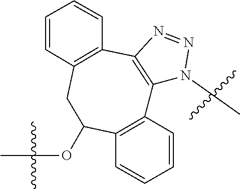 [SMILES: *OC1Cc2ccccc2-c2nnn(*)c2-c2ccccc21] |
| a cyclooctene | a diaryl tetrazine | 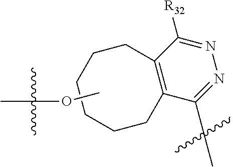 |
| | | or |
| | | 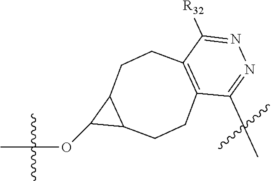 |
| a diaryl tetrazine | a cyclooctene | 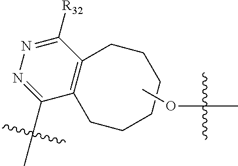 |
| | | or |
| | | 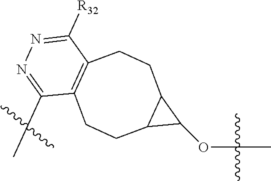 |
| a monoaryl tetrazine | a norbornene | 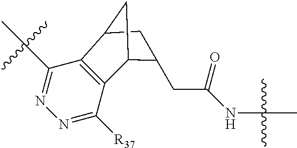 |
| a norbornene | a monoaryl tetrazine | 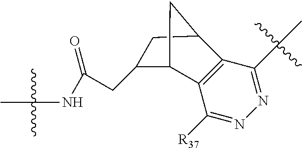 |
| an aldehyde | a hydroxylamine | 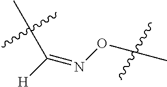 [SMILES: */C=N\O*] |
| an aldehyde | a hydrazine | 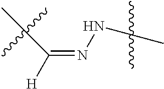 [SMILES: */C=N\N*] |
| an aldehyde | NH₂—NH—C(═O)— | 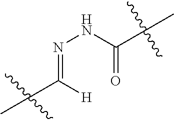 [SMILES: */C=N/NC(*)=O] |
| a ketone | a hydroxylamine | 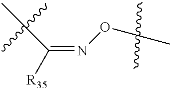 |
| a ketone | a hydrazine | 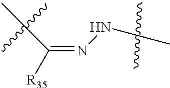 |
| a ketone | NH₂—NH—C(═O)— | 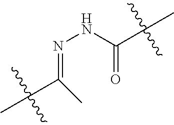 [SMILES: *C(=O)N/N=C(/*)C] |
| a hydroxylamine | an aldehyde | 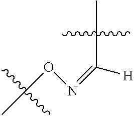 [SMILES: */C=N\O*] |
| a hydroxylamine | a ketone | 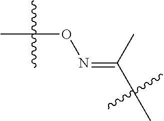 [SMILES: *O/N=C(/*)C] |
| a hydrazine | an aldehyde | 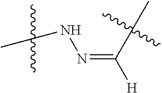 [SMILES: */C=N\N*] |
| a hydrazine | a ketone | 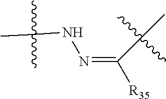 |
| NH₂—NH—C(═O)— | an aldehyde | 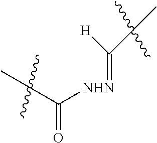 |
| NH₂—NH—C(═O)— | a ketone | 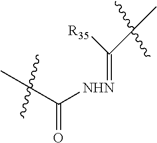 |
| a haloacetamide | a thiol | 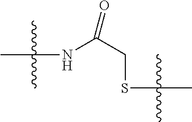 [SMILES: *NC(=O)CS*] |
| a maleimide | a thiol | 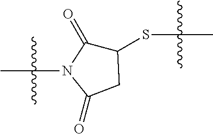 [SMILES: *SC1CC(=O)N(*)C1=O] |
| a vinyl sulfone | a thiol | 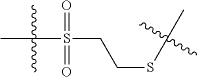 [SMILES: *SCCS(*)(=O)=O] |
| a thiol | a vinyl sulfone | 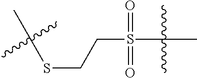 [SMILES: *SCCS(*)(=O)=O] |
| an aziridine | a thiol | 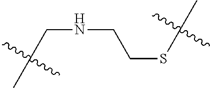 [SMILES: *CNCCS*] |
| | | or |
| | | 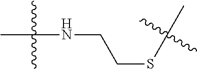 [SMILES: *NCCS*] |
| a thiol | an aziridine | 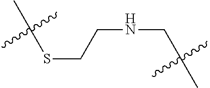 [SMILES: *CNCCS*] |
| | | or |
| | | 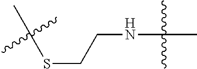 [SMILES: *NCCS*] |
| 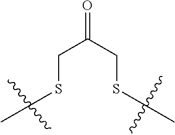 [SMILES: *SCC(=O)CS*] | hydroxylamine | 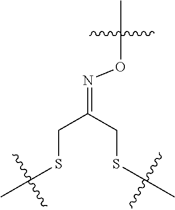 [SMILES: *ON=C(CS*)CS*] |
| 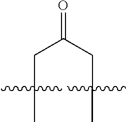 [SMILES: *CC(=O)C*] | hydroxylamine | 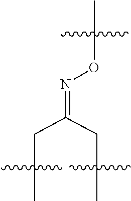 [SMILES: *CC(C*)=NO*] |
| 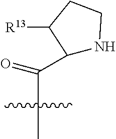 | 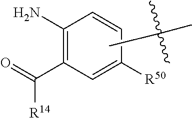 | 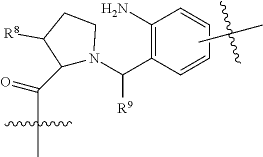 |
| 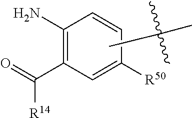 | 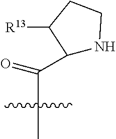 | 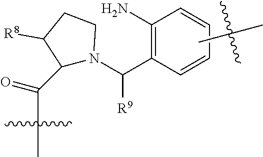 |
| 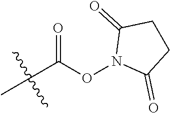 [SMILES: *C(=O)ON1C(=O)CCC1=O] | —NH₂, | amide |
| 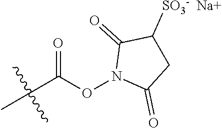 | | |
| 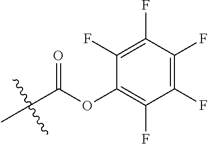 [SMILES: *C(=O)Oc1c(F)c(F)c(F)c(F)c1F] | | |
| 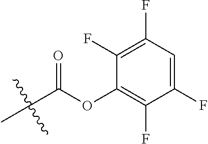 [SMILES: *C(=O)Oc1c(F)c(F)cc(F)c1F] | | |
| 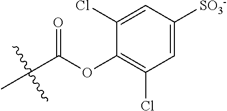 | | |
| —NH₂, | 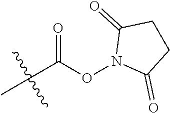 [SMILES: *C(=O)ON1C(=O)CCC1=O] | amide |
| | 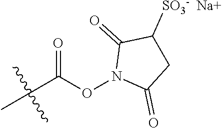 | |
| | 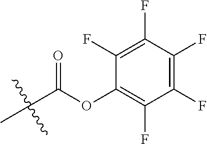 [SMILES: *C(=O)Oc1c(F)c(F)c(F)c(F)c1F] | |
| | 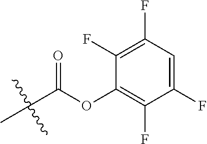 [SMILES: *C(=O)Oc1c(F)c(F)cc(F)c1F] | |
| | 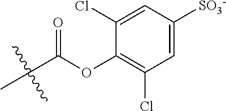 | |
| CoA or CoA analogue | Serine residue | 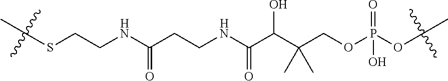 [SMILES: *OP(=O)(O)OCC(C)(C)C(O)C(=O)NCCC(=O)NCCS*] |
| | | 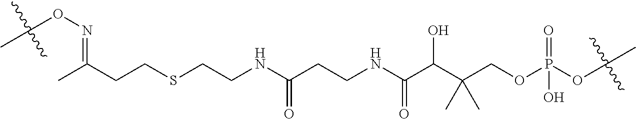 [SMILES: *O/N=C(\C)CCSCCNC(=O)CCNC(=O)C(O)C(C)(C)COP(=O)(O)O*] |
| | | 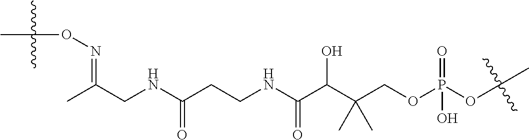 [SMILES: *O/N=C(\C)CNC(=O)CCNC(=O)C(O)C(C)(C)COP(=O)(O)O*] |
| | | 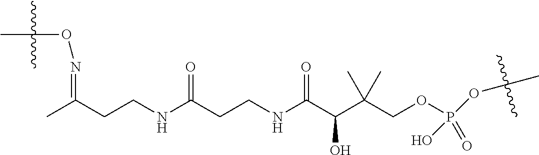 [SMILES: *O/N=C(\C)CCNC(=O)CCNC(=O)[C@H](O)C(C)(C)COP(=O)(O)O*] |
| | | 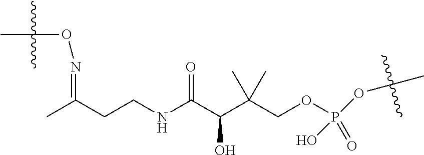 [SMILES: *O/N=C(\C)CCNC(=O)[C@H](O)C(C)(C)COP(=O)(O)O*] |
| | | 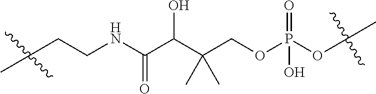 [SMILES: *CCNC(=O)C(O)C(C)(C)COP(=O)(O)O*] |
| | | 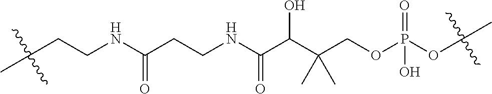 [SMILES: *CCNC(=O)CCNC(=O)C(O)C(C)(C)COP(=O)(O)O*] |
| | | 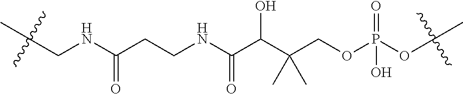 [SMILES: *CNC(=O)CCNC(=O)C(O)C(C)(C)COP(=O)(O)O*] |
| pyridyldithiol | thiol | disulfide |

where: R³² in Table F is H, C₁₋₄ alkyl, phenyl, pyrimidine or pyridine; R³⁵ in Table F is H, C₁₋₆alkyl, phenyl or C₁₋₄alkyl substituted with 1 to 3 —OH groups; each R⁷ in Table F is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; R³⁷ in Table F is independently selected from H, phenyl and pyridine; q in Table F is 0, 1, 2 or 3; R⁸ and R¹³ in Table F is H or methyl; and R⁹ and R¹⁴ in Table F is H, —CH₃ or phenyl; R in Table F is H or any suitable substituent; and R⁵⁰ in Table F is H.

In addition, a linker component can be a group listed in Table G below.

TABLE G

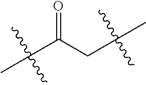 [SMILES: *CC(*)=O]

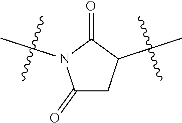 [SMILES: *C1CC(=O)N(*)C1=O]

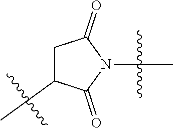 [SMILES: *C1CC(=O)N(*)C1=O]

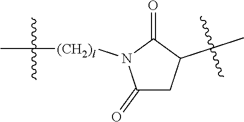

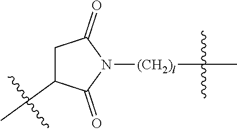

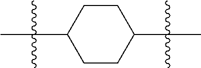 [SMILES: *C1CCC(*)CC1]

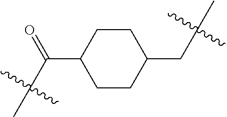 [SMILES: *CC1CCC(C(*)=O)CC1]

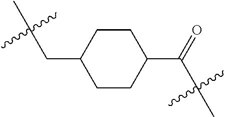 [SMILES: *CC1CCC(C(*)=O)CC1]

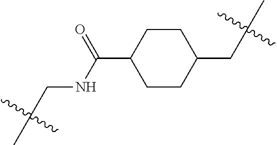 [SMILES: *CNC(=O)C1CCC(C*)CC1]

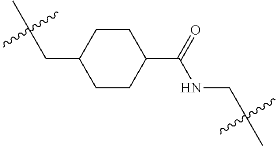 [SMILES: *CNC(=O)C1CCC(C*)CC1]

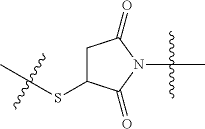 [SMILES: *SC1CC(=O)N(*)C1=O]

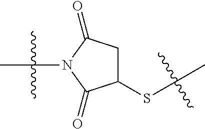 [SMILES: *SC1CC(=O)N(*)C1=O]

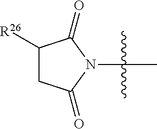

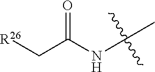

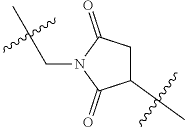 [SMILES: *CN1C(=O)CC(*)C1=O]

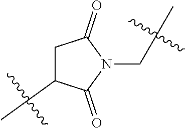 [SMILES: *CN1C(=O)CC(*)C1=O]

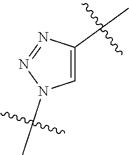 [SMILES: *c1cn(*)nn1]

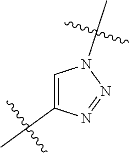 [SMILES: *c1cn(*)nn1]

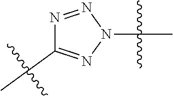 [SMILES: *c1nnn(*)n1]

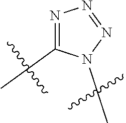 [SMILES: *c1nnnn1*]

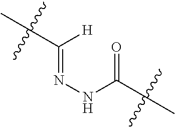 [SMILES: */C=N/NC(*)=O]

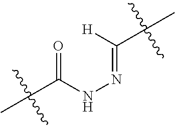 [SMILES: */C=N/NC(*)=O]

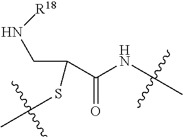

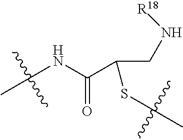

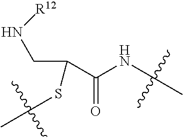

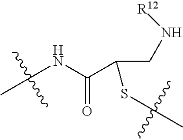

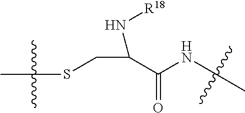

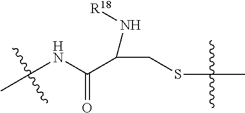

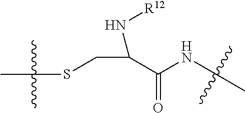

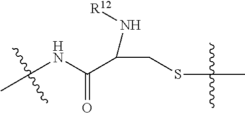

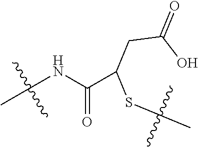 [SMILES: *NC(=O)C(CC(=O)O)S*]

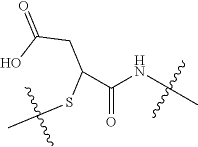 [SMILES: *NC(=O)C(CC(=O)O)S*]

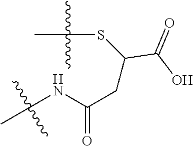 [SMILES: *NC(=O)CC(S*)C(=O)O]

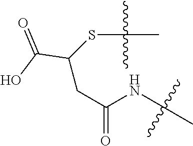 [SMILES: *NC(=O)CC(S*)C(=O)O]

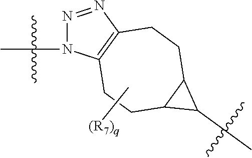

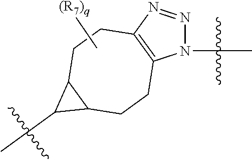

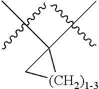 [SMILES: *C1(*)CC1]

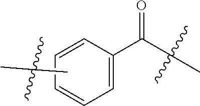

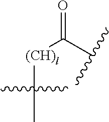

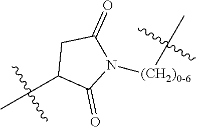 [SMILES: *CN1C(=O)CC(*)C1=O]

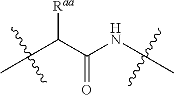

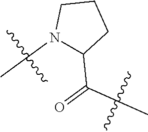 [SMILES: *C(=O)C1CCCN1*]

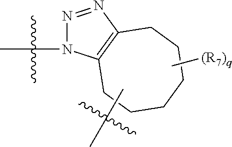

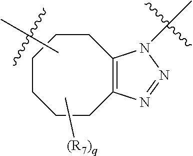

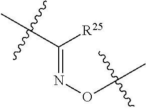

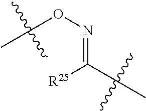

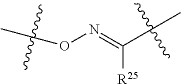

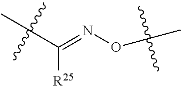

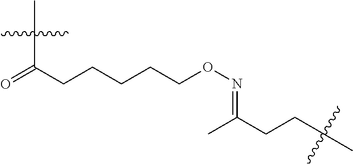 [SMILES: *CC/C(C)=N/OCCCCCC(*)=O]

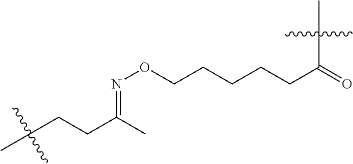 [SMILES: *CC/C(C)=N/OCCCCCC(*)=O]

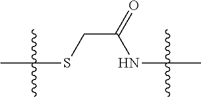 [SMILES: *NC(=O)CS*]

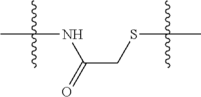 [SMILES: *NC(=O)CS*]

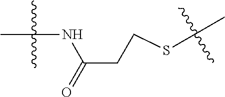 [SMILES: *NC(=O)CCS*]

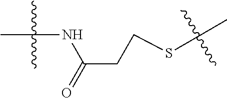 [SMILES: *NC(=O)CCS*]

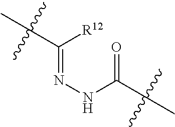

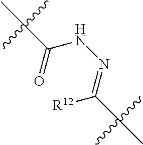

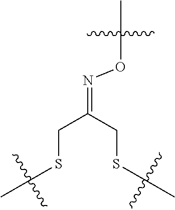 [SMILES: *ON=C(CS*)CS*]

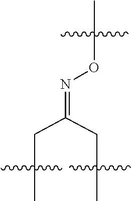 [SMILES: *CC(C*)=NO*]

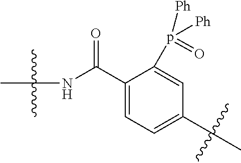

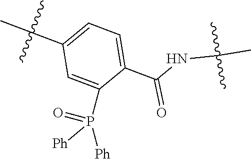 [SMILES: *NC(=O)c1ccc(*)cc1P(=O)(c1ccccc1)c1ccccc1]

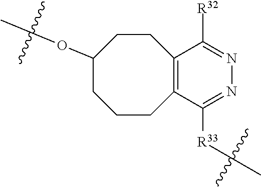

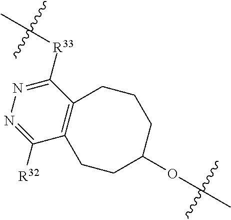

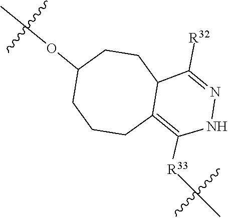

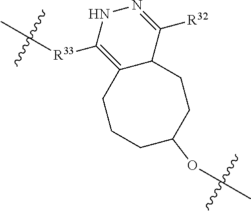

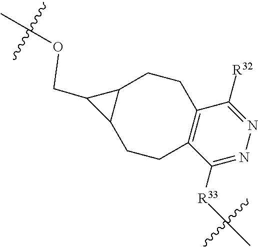

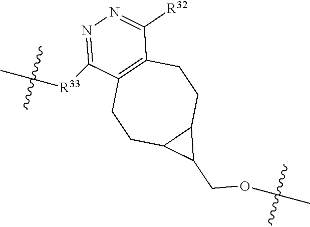

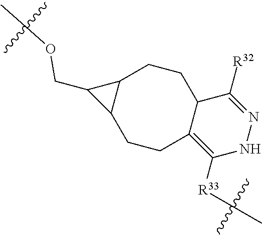

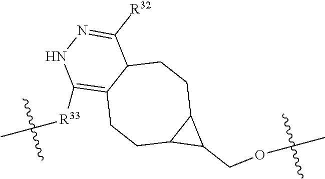

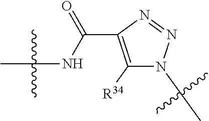

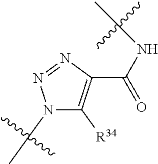

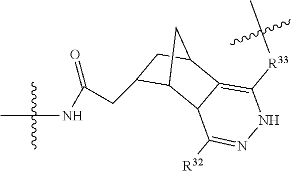

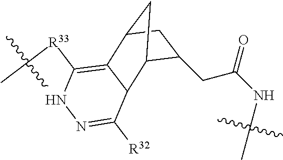

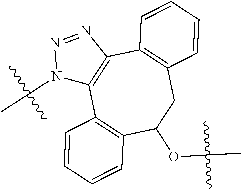 [SMILES: *OC1Cc2ccccc2-c2nnn(*)c2-c2ccccc21]

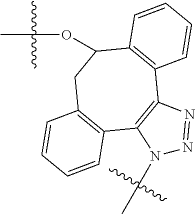 [SMILES: *OC1Cc2ccccc2-c2c(nnn2*)-c2ccccc21]

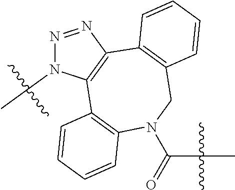 [SMILES: *C(=O)N1Cc2ccccc2-c2nnn(*)c2-c2ccccc21]

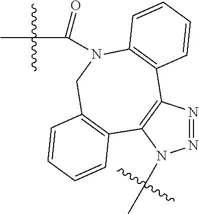 [SMILES: *C(=O)N1Cc2ccccc2-c2c(nnn2*)-c2ccccc21]

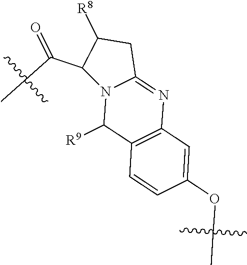

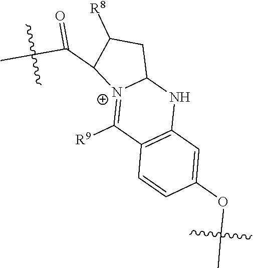

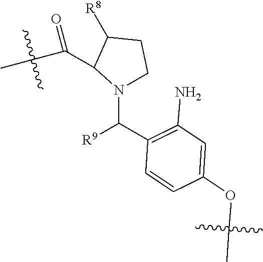

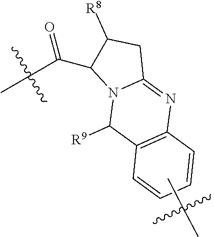

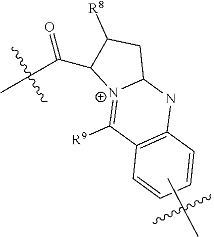

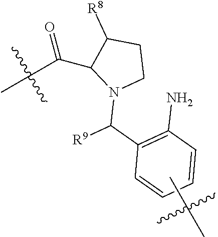

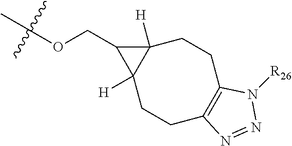

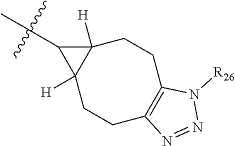

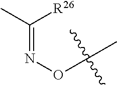

each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each R¹² is independently selected from H and C₁-C₆alkyl
R⁸ is H or methyl;
R⁹ is H , —CH₃ or phenyl;
each R²⁵ is independently selected from H or C₁₋₄alkyl;
each R¹⁸ is independently selected from a C₁-C₆alkyl, a C₁-C₆alkyl which is substituted with azido and a C₁-C₆alkyl which is substituted with a 1 to 5 hydroxyl;
q is 0, 1, 2 or 3;
l is 1, 2, 3, 4, 5 or 6;
R²⁶ is 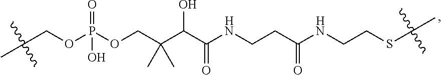 [SMILES: *COP(=O)(O)OCC(C)(C)C(O)C(=O)NCCC(=O)NCCS*] 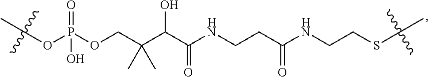 [SMILES: *OP(=O)(O)OCC(C)(C)C(O)C(=O)NCCC(=O)NCCS*] 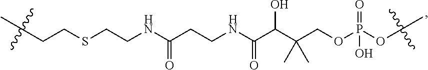 [SMILES: *CCSCCNC(=O)CCNC(=O)C(O)C(C)(C)COP(=O)(O)O*] 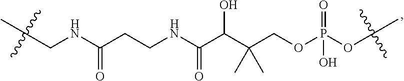 [SMILES: *CNC(=O)CCNC(=O)C(O)C(C)(C)COP(=O)(O)O*] 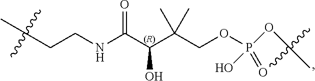 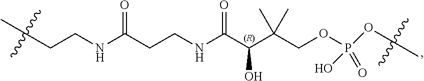 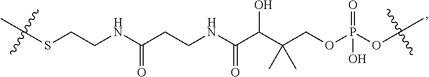 [SMILES: *OP(=O)(O)OCC(C)(C)C(O)C(=O)NCCC(=O)NCCS*] 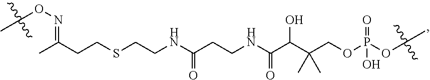 [SMILES: *O/N=C(\C)CCSCCNC(=O)CCNC(=O)C(O)C(C)(C)COP(=O)(O)O*] 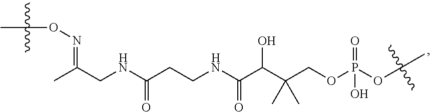 [SMILES: *O/N=C(\C)CNC(=O)CCNC(=O)C(O)C(C)(C)COP(=O)(O)O*] 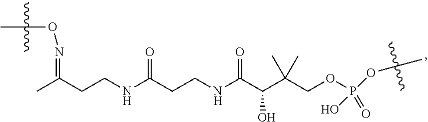 [SMILES: *O/N=C(\C)CCNC(=O)CCNC(=O)[C@@H](O)C(C)(C)COP(=O)(O)O*] 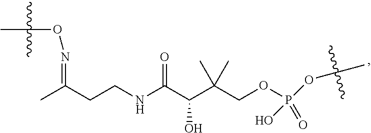 [SMILES: *O/N=C(\C)CCNC(=O)[C@@H](O)C(C)(C)COP(=O)(O)O*] 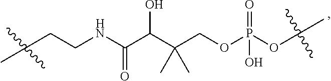 [SMILES: *CCNC(=O)C(O)C(C)(C)COP(=O)(O)O*] 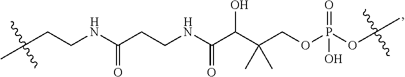 [SMILES: *CCNC(=O)CCNC(=O)C(O)C(C)(C)COP(=O)(O)O*]
or 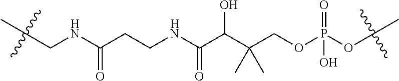 [SMILES: *CNC(=O)CCNC(=O)C(O)C(C)(C)COP(=O)(O)O*]
R³² is independently selected from H, C₁₋₄ alkyl, phenyl, pyrimidine and pyridine;
R³³ is independently selected from 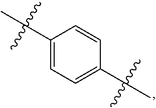 [SMILES: *c1ccc(*)cc1] 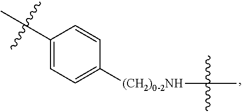 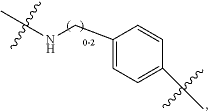 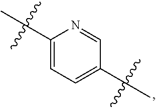 [SMILES: *c1ccc(*)nc1] 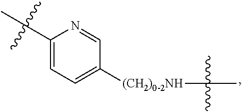 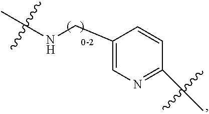 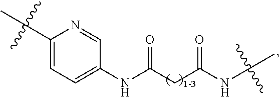
and 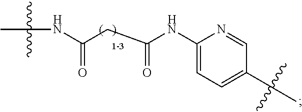
R³⁴ is independently selected from H, C₁₋₄ alkyl, and C₁₋₆ haloalkyl, and
R^{aa} is an amino acid side chain.

As used herein, when a partial structure of a compound is illustrated, a wavy line ( [symbol] ) indicates the point of attachment of the partial structure to the rest of the molecule.

As used herein, when a partial structure of a compound is illustrated, a wavy line ( [symbol] ) indicates the point of attachment of the partial structure to the rest of the molecule.

The term “self-immolative spacer” as used herein, refers to a moiety comprising one or more triggering groups (TG) which are activated by acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage, and after activation the protecting group is removed, which generates a cascade of disassembling reactions leading to the temporally sequential release of a leaving group. Such cascade of reactions can be, but not limited to, 1,2-, 1,4-, 1,6- or 1,8-elimination reactions.

Non-limiting examples of self-immolative spacer include:

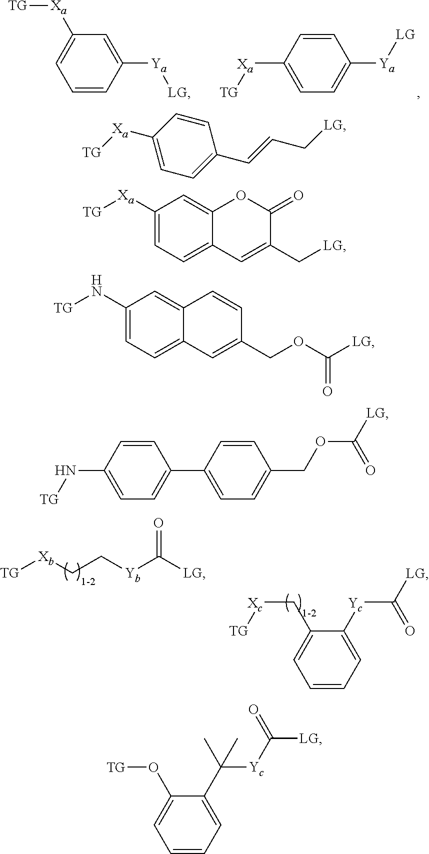

TG-YₐLG and TG-Xₐ-LG wherein such groups can be optionally substituted, and
wherein:
   TG is a triggering group;
   Xₐ is O, NH or S;
   X_{b} is O, NH, NCH₃ or S;
   X_{c} is O or NH;
   Yₐ is CH₂, CH₂O or CH₂NH;
   Y_{b} is CH₂, O or NH;
   Y_{c} is a bond, CH₂, O or NH, and
   LG is a leaving group such as a Drug moiety (D) of the Linker-Drug group of the invention.

Additional non-limiting examples of self-immolative spacers are described in Angew. Chem. Int. Ed. 2015, 54, 7492-7509.

In certain embodiments the self-immolative spacer connected to a drug moiety is a moiety having the following structure:

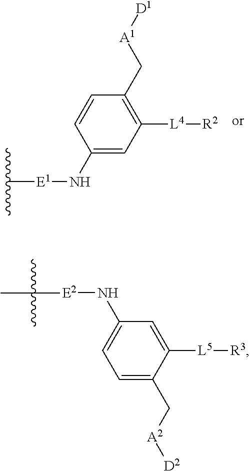

wherein E¹ and/or E² is an enzyme cleavage element, and A¹, A², D¹, D², R², R³, L³, and L⁴ are as defined herein.

In a certain embodiment the self-immolative spacer is moiety having the structure

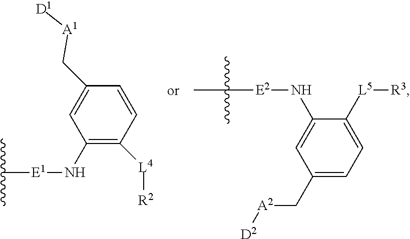

where E¹ and/or E² is a bivalent peptide spacer, R² and/or R³ is an enzyme cleavage element, and A¹, A², D¹, D², L³, L⁴, R² and R³ are as defined herein.

The term “self-immolative group,” as used herein, refer to a group that can generate a cascade of disassembling reactions leading to the temporally sequential release of a leaving group when the TG is activated and removed.

In some embodiments, the self-immolative group is a group having the structure

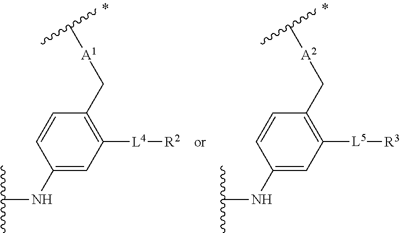

wherein A¹, A², R², R³, L³, and L⁴ are as defined herein, [symbol] indicates the point of attachment to E¹ or E²; and [symbol] indicates the point of attachment to D¹ or D².

### Drug Moieties

In some embodiments, an intermediate, which is the precursor of the linker moiety, is reacted with the drug moiety (which is not a BH3 mimetic such as a Mcl-1 inhibitor, a Bcl-2 inhibitor or a Bcl-xL inhibitor) under appropriate conditions. In some embodiments, reactive groups are used on the drug and/or the intermediate or linker. The product of the reaction between the drug and the intermediate, or the derivatized drug (drug plus linker), is subsequently reacted with the antibody or antigen-binding fragment under conditions that facilitate conjugation of the drug and intermediate or derivatized drug and antibody or antigen-binding fragment. Alternatively, the intermediate or linker may first be reacted with the antibody or antigen-binding fragment, or a derivatized antibody or antigen-binding fragment, and then reacted with the drug or derivatized drug.

A number of different reactions are available for covalent attachment of the drug moiety and/or linker moiety to the antibody or antigen-binding fragment. This is often accomplished by reaction of one or more amino acid residues of the antibody or antigen-binding fragment, including the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a drug moiety to an amino (or carboxy) group on an antibody or antigen-binding fragment. Additionally, bifunctional agents such as dialdehydes or imidoesters may also be used to link the amino group on a drug moiety to an amino group on an antibody or antigen-binding fragment. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

### Topoisomerase 1 Inhibitors

In some embodiments, one of D1 and D2 comprises a topoisomerase 1 inhibitor, or an enantiomer, diastereoisomer, atropisomer, deuterated derivative, and/or addition salt thereof with a pharmaceutically acceptable acid or base.

In some embodiments, D1 and/or D2 comprises a topoisomerase 1 inhibitor linked to the dual linker, wherein the topoisomerase 1 inhibitor is topotecan, exatecan, deruxtecan or SN-38. In some embodiments, D1 and/or D2 comprises a formula selected from the table below.

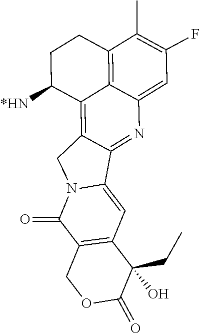

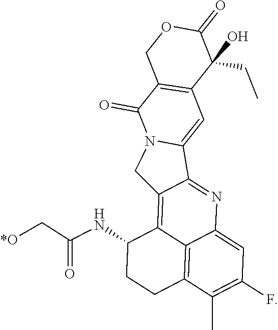

*represents the point of attachment to the remainder of the molecule

### Anti-Mitotic Drugs

In some embodiments, one of D1 and D2 comprises an anti-mitotic drug, or an enantiomer, diastereoisomer, atropisomer, deuterated derivative, and/or addition salt thereof with a pharmaceutically acceptable acid or base.

In some embodiments, one of D1 and D2 comprises an anti-mitotic drug linked to the dual linker, wherein the anti-mitotic drug is monomethyl auristatin E (MMAE) or a taxane. In one embodiment, the taxane is selected from docetaxel, paclitaxel, or cabazitaxel.

### Drug Loading

Drug loading is represented by p (or 2a in the compounds and conjugates of the present disclosure), and is also referred to herein as the drug-to-antibody ratio (DAR). Drug loading may range from 2 to 32 drug moieties per antibody or antigen-binding fragment. In some embodiments, a is an integer from 1 to 16. In some embodiments, a is an integer from 1 to, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, a is an integer from 2 to 10, 2 to 9, 2 to 8, 2 to 7, 2 to 6, 2 to 5, 2 to 4, or 2 to 3. In some embodiments, a is an integer from 1 to 16. In some embodiments, a is an integer from 1 to 8. In some embodiments, a is an integer from 1 to 5. In some embodiments, a is an integer from 2 to 4. In some embodiments, a is 1, 2, 3, 4, 5, 6, 7, or 8. In some embodiments, a is 2. In some embodiments, a is 4.

Drug loading may be limited by the number of attachment sites on the antibody or antigen-binding fragment. In some embodiments, the linker moiety (L) of the ADC attaches to the antibody or antigen-binding fragment through a chemically active group on one or more amino acid residues on the antibody or antigen-binding fragment. For example, the linker may be attached to the antibody or antigen-binding fragment via a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, or to the sulfhydryl group of one or more cysteine residues). The site to which the linker is attached can be a natural residue in the amino acid sequence of the antibody or antigen-binding fragment, or it can be introduced into the antibody or antigen-binding fragment, e.g., by DNA recombinant technology (e.g., by introducing a cysteine residue into the amino acid sequence) or by protein biochemistry (e.g., by reduction, pH adjustment, or hydrolysis).

In some embodiments, the number of drug moieties that can be conjugated to an antibody or antigen-binding fragment is limited by the number of free cysteine residues. For example, where the attachment is a cysteine thiol group, an antibody may have only one or a few cysteine thiol groups, or may have only one or a few sufficiently reactive thiol groups through which a linker may be attached. Generally, antibodies do not contain many free and reactive cysteine thiol groups that may be linked to a drug moiety. Indeed, most cysteine thiol residues in antibodies are involved in either interchain or intrachain disulfide bonds. Conjugation to cysteines can therefore, in some embodiments, require at least partial reduction of the antibody. Over-attachment of linker-toxin to an antibody may destabilize the antibody by reducing the cysteine residues available to form disulfide bonds. Therefore, an optimal drug:antibody ratio should increase potency of the ADC (by increasing the number of attached drug moieties per antibody) without destabilizing the antibody or antigen-binding fragment. In some embodiments, an optimal ratio may be 2, 4, 6, or 8. In some embodiments, an optimal ratio may be 2 or 4.

In some embodiments, an antibody or antigen-binding fragment is exposed to reducing conditions prior to conjugation in order to generate one or more free cysteine residues. An antibody, in some embodiments, may be reduced with a reducing agent such as dithiothreitol (DTT) or tris(2-carboxyethyl)phosphine (TCEP), under partial or total reducing conditions, to generate reactive cysteine thiol groups. Unpaired cysteines may be generated through partial reduction with limited molar equivalents of TCEP, which can reduce the interchain disulfide bonds which link the light chain and heavy chain (one pair per H-L pairing) and the two heavy chains in the hinge region (two pairs per H—H pairing in the case of human IgG1) while leaving the intrachain disulfide bonds intact (Stefano et al. (2013) Methods Mol Biol. 1045:145-71). In embodiments, disulfide bonds within the antibodies are reduced electrochemically, e.g., by employing a working electrode that applies an alternating reducing and oxidizing voltage. This approach can allow for on-line coupling of disulfide bond reduction to an analytical device (e.g., an electrochemical detection device, an NMR spectrometer, or a mass spectrometer) or a chemical separation device (e.g., a liquid chromatograph (e.g., an HPLC) or an electrophoresis device (see, e.g., US 2014/0069822)). In some embodiments, an antibody is subjected to denaturing conditions to reveal reactive nucleophilic groups on amino acid residues, such as cysteine.

The drug loading of an ADC may be controlled in different ways, e.g., by: (i) limiting the molar excess of drug-linker intermediate or linker reagent relative to antibody; (ii) limiting the conjugation reaction time or temperature; (iii) partial or limiting reductive conditions for cysteine thiol modification; and/or (iv) engineering by recombinant techniques the amino acid sequence of the antibody such that the number and position of cysteine residues is modified for control of the number and/or position of linker-drug attachments.

In some embodiments, free cysteine residues are introduced into the amino acid sequence of the antibody or antigen-binding fragment. For example, cysteine engineered antibodies can be prepared wherein one or more amino acids of a parent antibody are replaced with a cysteine amino acid. Any form of antibody may be so engineered, i.e. mutated. For example, a parent Fab antibody fragment may be engineered to form a cysteine engineered Fab referred to as a “ThioFab.” Similarly, a parent monoclonal antibody may be engineered to form a “ThioMab.” A single site mutation yields a single engineered cysteine residue in a ThioFab, whereas a single site mutation yields two engineered cysteine residues in a ThioMab, due to the dimeric nature of the IgG antibody. DNA encoding an amino acid sequence variant of the parent polypeptide can be prepared by a variety of methods known in the art (see, e.g., the methods described in WO 2006/034488). These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may also be constructed by restriction fragment manipulation or by overlap extension PCR with synthetic oligonucleotides. ADCs of Formula (1) include, but are not limited to, antibodies that have 1, 2, 3, or 4 engineered cysteine amino acids (Lyon et al. (2012) Methods Enzymol. 502:123-38). In some embodiments, one or more free cysteine residues are already present in an antibody or antigen-binding fragment, without the use of engineering, in which case the existing free cysteine residues may be used to conjugate the antibody or antigen-binding fragment to a drug moiety.

Where more than one nucleophilic group reacts with a drug-linker intermediate or a linker moiety reagent followed by drug moiety reagent, in a reaction mixture comprising multiple copies of the antibody or antigen-binding fragment and linker moiety, then the resulting product can be a mixture of ADC compounds with a distribution of one or more drug moieties attached to each copy of the antibody or antigen-binding fragment in the mixture. In some embodiments, the drug loading in a mixture of ADCs resulting from a conjugation reaction ranges from 1 to 16 drug moieties attached per antibody or antigen-binding fragment. The average number of drug moieties per antibody or antigen-binding fragment (i.e., the average drug loading, or average p) may be calculated by any conventional method known in the art, e.g., by mass spectrometry (e.g., liquid chromatography-mass spectrometry (LC-MS)) and/or high-performance liquid chromatography (e.g., HIC-HPLC). In some embodiments, the average number of drug moieties per antibody or antigen-binding fragment is determined by liquid chromatography-mass spectrometry (LC-MS). In some embodiments, the average number of drug moieties per antibody or antigen-binding fragment is from about 1.5 to about 3.5, about 2.5 to about 4.5, about 3.5 to about 5.5, about 4.5 to about 6.5, about 5.5 to about 7.5, about 6.5 to about 8.5, or about 7.5 to about 9.5. In some embodiments, the average number of drug moieties per antibody or antigen-binding fragment is from about 2 to about 4, about 3 to about 5, about 4 to about 6, about 5 to about 7, about 6 to about 8, about 7 to about 9, about 2 to about 8, or about 4 to about 8.

In some embodiments, the average number of drug moieties per antibody or antigen-binding fragment is about 2. In some embodiments, the average number of drug moieties per antibody or antigen-binding fragment is about 1.5, about 1.6, about 1.7, about 1.8, about 1.9, about 2, about 2.1, about 2.2, about 2.3, about 2.4, or about 2.5. In some embodiments, the average number of drug moieties per antibody or antigen-binding fragment is 2.

In some embodiments, the average number of drug moieties per antibody or antigen-binding fragment is about 4. In some embodiments, the average number of drug moieties per antibody or antigen-binding fragment is about 3.5, about 3.6, about 3.7, about 3.8, about 3.9, about 4, about 4.1, about 4.2, about 4.3, about 4.4, or about 4.5. In some embodiments, the average number of drug moieties per antibody or antigen-binding fragment is 4.

In some embodiments, the term “about,” as used with respect to the average number of drug moieties per antibody or antigen-binding fragment, means plus or minus 20%, 15%, 10%, 5%, or 1%. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Individual ADC compounds, or “species,” may be identified in the mixture by mass spectroscopy and separated by, e.g., UPLC or HPLC, e.g. hydrophobic interaction chromatography (HIC-HPLC). In some embodiments, a homogeneous or nearly homogenous ADC product with a single loading value may be isolated from the conjugation mixture, e.g., by electrophoresis or chromatography.

In some embodiments, higher drug loading (e.g., p>16) may cause aggregation, insolubility, toxicity, or loss of cellular permeability of certain antibody-drug conjugates. Higher drug loading may also negatively affect the pharmacokinetics (e.g., clearance) of certain ADCs.

In some embodiments, lower drug loading (e.g., p<2) may reduce the potency of certain ADCs against target-expressing cells. In some embodiments, the drug loading for an ADC of the present disclosure ranges from about 2 to about 16, about 2 to about 10, about 2 to about 8; from about 2 to about 6; from about 2 to about 5; from about 3 to about 5; from about 2 to about 4; or from about 4 to about 8.

In some embodiments, a drug loading and/or an average drug loading of about 2 is achieved, e.g., using partial reduction of intrachain disulfides on the antibody or antigen-binding fragment, and provides beneficial properties. In some embodiments, a drug loading and/or an average drug loading of about 4 or about 6 or about 8 is achieved, e.g., using partial reduction of intrachain disulfides on the antibody or antigen-binding fragment, and provides beneficial properties. In some embodiments, a drug loading and/or an average drug loading of less than about 2 may result in an unacceptably high level of unconjugated antibody species, which can compete with the ADC for binding to a target antigen and/or provide for reduced treatment efficacy. In some embodiments, a drug loading and/or average drug loading of more than about 16 may result in an unacceptably high level of product heterogeneity and/or ADC aggregation. A drug loading and/or an average drug loading of more than about 16 may also affect stability of the ADC, due to loss of one or more chemical bonds required to stabilize the antibody or antigen-binding fragment.

The present disclosure includes methods of producing the described ADCs. Briefly, the ADCs comprise an antibody or antigen-binding fragment as the antibody or antigen-binding fragment, a drug moiety, and a linker that joins the drug moiety and the antibody or antigen-binding fragment. In some embodiments, the ADCs can be prepared using a linker having reactive functionalities for covalently attaching to the drug moiety and to the antibody or antigen-binding fragment. In some embodiments, the antibody or antigen-binding fragment is functionalized to prepare a functional group that is reactive with a linker or a drug-linker intermediate. For example, in some embodiments, a cysteine thiol of an antibody or antigen-binding fragment can form a bond with a reactive functional group of a linker or a drug-linker intermediate to make an ADC. In some embodiments, an antibody or antigen-binding fragment is prepared with bacterial transglutaminase (BTG)-reactive glutamines specifically functionalized with an amine containing cyclooctyne BCN (N-[(1R,8S,9s)-Bicyclo[6.1.0]non-4-yn-9-ylmethyloxycarbonyl]-1,8-diamino-3,6-dioxaoctane) moiety. In some embodiments, site-specific conjugation of a linker or a drug-linker intermediate to a BCN moiety of an antibody or antigen-binding fragment is performed, e.g., as described and exemplified herein. The generation of the ADCs can be accomplished by techniques known to the skilled artisan.

In some embodiments, an ADC is produced by contacting an antibody or antigen-binding fragment with a linker and a drug moiety in a sequential manner, such that the antibody or antigen-binding fragment is covalently linked to the linker first, and then the pre-formed antibody-linker intermediate reacts with the drug moiety. The antibody-linker intermediate may or may not be subjected to a purification step prior to contacting the drug moiety. In other embodiments, an ADC is produced by contacting an antibody or antigen-binding fragment with a linker-drug compound pre-formed by reacting a linker with a drug moiety. The pre-formed linker-drug compound may or may not be subjected to a purification step prior to contacting the antibody or antigen-binding fragment. In other embodiments, the antibody or antigen-binding fragment contacts the linker and the drug moiety in one reaction mixture, allowing simultaneous formation of the covalent bonds between the antibody or antigen-binding fragment and the linker, and between the linker and the drug moiety. This method of producing ADCs may include a reaction, wherein the antibody or antigen-binding fragment contacts the antibody or antigen-binding fragment prior to the addition of the linker to the reaction mixture, and vice versa. In some embodiments, an ADC is produced by reacting an antibody or antigen-binding fragment with a linker joined to a drug moiety under conditions that allow conjugation.

The ADCs prepared according to the methods described above may be subjected to a purification step. The purification step may involve any biochemical methods known in the art for purifying proteins, or any combination of methods thereof. These include, but are not limited to, tangential flow filtration (TFF), affinity chromatography, ion exchange chromatography, any charge or isoelectric point-based chromatography, mixed mode chromatography, e.g., CHT (ceramic hydroxyapatite), hydrophobic interaction chromatography, size exclusion chromatography, dialysis, filtration, selective precipitation, or any combination thereof.

### Therapeutic Uses and Compositions

Disclosed herein are methods of using the compositions described herein, e.g., the disclosed ADC compounds and compositions, in treating a subject for a disorder, e.g., a cancer. Compositions, e.g., ADCs, may be administered alone or in combination with at least one additional inactive and/or active agent, e.g., at least one additional therapeutic agent, and may be administered in any pharmaceutically acceptable formulation, dosage, and dosing regimen. Treatment efficacy may be evaluated for toxicity as well as indicators of efficacy and adjusted accordingly. Efficacy measures include, but are not limited to, a cytostatic and/or cytotoxic effect observed in vitro or in vivo, reduced tumor volume, tumor growth inhibition, and/or prolonged survival.

Methods of determining whether an ADC exerts a cytostatic and/or cytotoxic effect on a cell are known. For example, the cytotoxic or cytostatic activity of an ADC can be measured by, e.g., exposing mammalian cells expressing a target antigen of the ADC in a cell culture medium; culturing the cells for a period from about 6 hours to about 6 days; and measuring cell viability (e.g., using a CellTiter-Glo® (CTG) or MTT cell viability assay). Cell-based in vitro assays may also be used to measure viability (proliferation), cytotoxicity, and induction of apoptosis (caspase activation) of the ADC.

For determining cytotoxicity, necrosis or apoptosis (programmed cell death) may be measured. Necrosis is typically accompanied by increased permeability of the plasma membrane, swelling of the cell, and rupture of the plasma membrane. Apoptosis can be quantitated, for example, by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica (1999) 2:34-7 (Roche Molecular Biochemicals).

Apoptosis may also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al., eds. (1992) pp. 3.17.1-3.17.16). Cells also can be labeled with a DNA dye (e.g., acridine orange, ethidium bromide, or propidium iodide) and the cells observed for chromatin condensation and margination along the inner nuclear membrane. Apoptosis may also be determined, in some embodiments, by screening for caspase activity. In some embodiments, a Caspase-Glo® Assay can be used to measure activity of caspase-3 and caspase-7. In some embodiments, the assay provides a luminogenic caspase-3/7 substrate in a reagent optimized for caspase activity, luciferase activity, and cell lysis. In some embodiments, adding Caspase-Glo® 3/7 Reagent in an “add-mix-measure” format may result in cell lysis, followed by caspase cleavage of the substrate and generation of a “glow-type” luminescent signal, produced by luciferase. In some embodiments, luminescence may be proportional to the amount of caspase activity present, and can serve as an indicator of apoptosis. Other morphological changes that can be measured to determine apoptosis include, e.g., cytoplasmic condensation, increased membrane blebbing, and cellular shrinkage. Determination of any of these effects on cancer cells indicates that an ADC is useful in the treatment of cancers.

Cell viability may be measured, e.g., by determining in a cell the uptake of a dye such as neutral red, trypan blue, Crystal Violet, or ALAMAR™ blue (see, e.g., Page et al. (1993) Intl J Oncology 3:473-6). In such an assay, the cells are incubated in media containing the dye, the cells are washed, and the remaining dye, reflecting cellular uptake of the dye, is measured spectrophotometrically.

Cell viability may also be measured, e.g., by quantifying ATP, an indicator of metabolically active cells. In some embodiments, in vitro potency and/or cell viability of prepared ADCs may be assessed using a CellTiter-Glo® (CTG) cell viability assay, as described in the examples provided herein. In this assay, in some embodiments, the single reagent (CellTiter-Glo® Reagent) is added directly to cells cultured in serum-supplemented medium. The addition of reagent results in cell lysis and generation of a luminescent signal proportional to the amount of ATP present. The amount of ATP is directly proportional to the number of cells present in culture Cell viability may also be measured, e.g., by measuring the reduction of tetrazolium salts.

In some embodiments, in vitro potency and/or cell viability of prepared ADCs may be assessed using an MTT cell viability assay, as described in the examples provided herein. In this assay, in some embodiments, the yellow tetrazolium MTT (3-(4, 5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan can then be solubilized and quantified by spectrophotometric means.

In certain aspects, the present disclosure features a method of killing, inhibiting or modulating the growth of a cancer cell or tissue. In some embodiments, the cancer is a breast cancer, multiple myeloma, plasma cell myeloma, leukemia, lymphoma, sarcoma, gastric cancer, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer or head and neck cancer. In some embodiments, the cancer is a lymphoma or gastric cancer.

In some embodiments, the disclosed ADCs may be administered in any cell or tissue that expresses PCAD, such as a PCAD-expressing cancer cell or tissue. An exemplary embodiment includes a method of killing a PCAD-expressing cancer cell or tissue. The method may be used with any cell or tissue that expresses PCAD, such as a cancerous cell or a metastatic lesion.

Non-limiting examples of PCAD-expressing cancers include breast cancer, gastric cancer, endometrial cancer, ovarian cancer, pancreatic cancer, bladder cancer, prostate cancer, and melanoma (Vieira and Paredes (2015) Mol Cancer 14:178).

In some embodiments, the disclosed ADCs may be administered in any cell or tissue that expresses CD48, such as a CD48-expressing cancer cell or tissue. An exemplary embodiment includes a method of killing a CD48-expressing cancer cell or tissue. The method may be used with any cell or tissue that expresses CD48, such as a cancerous cell or a metastatic lesion. Non-limiting examples of CD48-expressing cancers include a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer.

In some embodiments, the disclosed ADCs may be administered in any cell or tissue that expresses CD74, such as a CD74-expressing cancer cell or tissue. An exemplary embodiment includes a method of killing a CD74-expressing cancer cell or tissue. The method may be used with any cell or tissue that expresses CD74, such as a cancerous cell or a metastatic lesion. Non-limiting examples of CD74-expressing cancers include a breast cancer, multiple myeloma, plasma cell myeloma, leukemia, lymphoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, prostate cancer, small cell lung cancer, or spleen cancer.

In some embodiments, the disclosed ADCs may be administered in any cell or tissue that expresses HER2, such as a HER2-expressing cancer cell or tissue. An exemplary embodiment includes a method of killing a HER2-expressing cancer cell or tissue. The method may be used with any cell or tissue that expresses HER2, such as a cancerous cell or a metastatic lesion. Non-limiting examples of HER2-expressing cancers include breast cancer, gastric cancer, bladder cancer, urothelial cell carcinoma, esophageal cancer, lung cancer (e.g., lung adenocarcinoma), uterine cancer (e.g., uterine serous endometrial carcinoma), salivary duct carcinoma, cervical cancer, endometrial cancer, and ovarian cancer (English et al. (2013) Mol Diagn Ther. 17:85-99). Non-limiting examples of HER2-expressing cells include HCC1954 and HCC2218 breast cancer cells, and cells comprising a recombinant nucleic acid encoding HER2 or a portion thereof.

In some embodiments, the disclosed ADCs may be administered in any cell or tissue that expresses EphA2, such as a EphA2-expressing cancer cell or tissue. An exemplary embodiment includes a method of killing a EphA2-expressing cancer cell or tissue. The method may be used with any cell or tissue that expresses EphA2, such as a cancerous cell or a metastatic lesion. Non-limiting examples of EphA2-expressing cancers include breast cancer, non-small cell lung cancer, pancreatic, esophageal, head and neck, stomach, bladder, and colon cancers. In some embodiments, EphA2-expressing cancer is breast cancer or non-small cell lung cancer.

In some embodiments, the disclosed ADCs may be administered in any cell or tissue that expresses TROP2, such as a TROP2-expressing cancer cell or tissue. An exemplary embodiment includes a method of killing a TROP2-expressing cancer cell or tissue. The method may be used with any cell or tissue that expresses TROP2, such as a cancerous cell or a metastatic lesion. Non-limiting examples of TROP2-expressing cancers include breast cancer, non-small cell lung cancer, pancreatic, esophageal, head and neck, stomach, bladder, and colon cancers. In some embodiments, TROP2-expressing cancer is breast cancer or non-small cell lung cancer.

In some embodiments, the disclosed ADCs may be administered in any cell or tissue that expresses B7-H3, such as a B7-H3-expressing cancer cell or tissue. An exemplary embodiment includes a method of killing a B7-H3-expressing cancer cell or tissue. The method may be used with any cell or tissue that expresses B7-H3, such as a cancerous cell or a metastatic lesion. Non-limiting examples of B7-H3-expressing cancers include breast cancer, non-small cell lung cancer, pancreatic, esophageal, head and neck, stomach, bladder, and colon cancers. In some embodiments, B7-H3-expressing cancer is breast cancer or non-small cell lung cancer.

In some embodiments, the disclosed ADCs may be administered in any cell or tissue that expresses 5T4, such as a 5T4-expressing cancer cell or tissue. An exemplary embodiment includes a method of killing a 5T4-expressing cancer cell or tissue. The method may be used with any cell or tissue that expresses 5T4, such as a cancerous cell or a metastatic lesion. Non-limiting examples of 5T4-expressing cancers include breast cancer, non-small cell lung cancer, pancreatic, esophageal, head and neck, stomach, bladder, and colon cancers. In some embodiments, 5T4-expressing cancer is breast cancer or non-small cell lung cancer.

Exemplary methods include the steps of contacting a cell with an ADC, as described herein, in an effective amount, i.e., an amount sufficient to kill the cell. The method can be used on cells in culture, e.g., in vitro, in vivo, ex vivo, or in situ. For example, cells that express HER2 (e.g., cells collected by biopsy of a tumor or metastatic lesion; cells from an established cancer cell line; or recombinant cells), can be cultured in vitro in culture medium and the contacting step can be affected by adding the ADC to the culture medium. The method will result in killing of cells expressing HER2, including in particular cancer cells expressing HER2. Alternatively, the ADC can be administered to a subject by any suitable administration route (e.g., intravenous, subcutaneous, or direct contact with a tumor tissue) to have an effect in vivo. This approach can be used for antibodies targeting other cell surface antigens (e.g., EGFR, CD7, HER2, CD48, CD74 or EphA2).

The in vivo effect of a disclosed ADC therapeutic composition can be evaluated in a suitable animal model. For example, xenogeneic cancer models can be used, wherein cancer explants or passaged xenograft tissues are introduced into immune compromised animals, such as nude or SCID mice (Klein et al. (1997) Nature Med. 3:402-8). Efficacy may be predicted using assays that measure inhibition of tumor formation, tumor regression or metastasis, and the like.

In vivo assays that evaluate the promotion of tumor death by mechanisms such as apoptosis may also be used. In some embodiments, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

Further provided herein, in some embodiments, are therapeutic uses for the described ADC compounds and compositions, e.g., in treating a cancer. In some embodiments, the present disclosure provides methods of treating a cancer (e.g., a cancer that expresses an antigen targeted by the antibody or antigen-binding fragment of the ADC, such as PCAD, HER2, CD48, CD74, EphA2, EGFR, CD7, HER2, TROP2, B7-H3 or 5T4, e.g. MET). In some embodiments, the present disclosure provides methods of reducing or slowing the expansion of a cancer cell population in a subject. In some embodiments, the present disclosure provides methods of determining whether a subject having or suspected of having a cancer will be responsive to treatment with an ADC compound or composition disclosed herein.

An exemplary embodiment is a method of treating a subject having or suspected of having a cancer, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer expresses a target antigen. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a method of reducing or inhibiting the growth of a tumor in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the tumor expresses a target antigen. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer, head and neck cancer, or spleen cancer. In some embodiments, the tumor is a gastric cancer. In some embodiments, administration of the antibody-drug conjugate, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%.

Another exemplary embodiment is a method of reducing or slowing the expansion of a cancer cell population in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer cell population expresses a target antigen. In some embodiments, the cancer cell population is from a tumor or a hematological cancer. In some embodiments, the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer cell population is from a lymphoma or gastric cancer. In some embodiments, administration of the antibody-drug conjugate, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%. In some embodiments, administration of the antibody-drug conjugate, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%.

Another exemplary embodiment is an antibody-drug conjugate, composition, or pharmaceutical composition (e.g., any of the exemplary antibody-drug conjugates, compositions, or pharmaceutical compositions disclosed herein) for use in treating a subject having or suspected of having a cancer. In some embodiments, the cancer expresses a target antigen. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in treating a subject having or suspected of having a cancer. In some embodiments, the cancer expresses a target antigen.

In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in a method of manufacturing a medicament for treating a subject having or suspected of having a cancer. In some embodiments, the cancer expresses a target antigen. In some embodiments, the target antigen is PCAD, HER2, CD48, CD74, EphA2, TROP2, B7-H3 or 5T4. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a method of determining whether a subject having or suspected of having a cancer will be responsive to treatment with an antibody-drug conjugate, composition, or pharmaceutical composition (e.g., any of the exemplary antibody-drug conjugates, compositions, or pharmaceutical compositions disclosed herein) by providing a biological sample from the subject; contacting the sample with the antibody-drug conjugate; and detecting binding of the antibody-drug conjugate to cancer cells in the sample. In some embodiments, the cancer cells in the sample express a target antigen.

In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the sample is a tissue biopsy sample, a blood sample, or a bone marrow sample.

In any of the embodiments of the present disclosure, the target antigen may be selected from BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor —like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74 EphA2, TROP2, B7-H3 or 5T4.

An exemplary embodiment is a method of treating a subject having or suspected of having a cancer, comprising administering to the subject a therapeutically effective amount of a composition disclosed herein, e.g., an ADC, composition, or pharmaceutical composition (e.g., any of the exemplary ADCs, compositions, or pharmaceutical compositions disclosed herein). In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor —like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74, EphA2, TROP2, B7-H3 or 5T4. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer.

Another exemplary embodiment is a method of delivering the drugs to a cell expressing TROP2, comprising conjugating the drugs to an antibody that immunospecifically binds to a TROP2 epitope and exposing the cell to the ADC. Exemplary cancer cells that express TROP2 for which the ADCs of the present disclosure are indicated include breast cancer or non-small cell lung cancer.

Another exemplary embodiment is a method of delivering the drugs to a cell expressing B7-H3, comprising conjugating the drugs to an antibody that immunospecifically binds to a B7-H3 epitope and exposing the cell to the ADC. Exemplary cancer cells that express B7-H3 for which the ADCs of the present disclosure are indicated include breast cancer or non-small cell lung cancer.

Another exemplary embodiment is a method of delivering the drugs to a cell expressing 5T4, comprising conjugating the drugs to an antibody that immunospecifically binds to a 5T4 epitope and exposing the cell to the ADC. Exemplary cancer cells that express 5T4 for which the ADCs of the present disclosure are indicated include breast cancer or non-small cell lung cancer. In certain aspects, the present disclosure further provides methods of reducing or inhibiting growth of a tumor (e.g., a CD48-expressing tumor, a CD74-expressing tumor, a PCAD-expressing tumor, an HER2-expressing tumor, a TROP2-expressing tumor, a B7-H3-expressing tumor, a 5T3-expressing tumor), comprising administering a therapeutically effective amount of an ADC or composition comprising an ADC. In some embodiments, the treatment is sufficient to reduce or inhibit the growth of the patient's tumor, reduce the number or size of metastatic lesions, reduce tumor load, reduce primary tumor load, reduce invasiveness, prolong survival time, and/or maintain or improve the quality of life. In some embodiments, the tumor is resistant or refractory to treatment with the antibody or antigen-binding fragment of the ADC (e.g., an anti-CD48 antibody, an anti-CD74 antibody, an anti-PCAD antibody, an anti-EphA2 antibody, an anti-HER2 antibody, an anti-TROP2 antibody, an anti-B7-H3 antibody, an anti-5T3 antibody) when administered alone.

An exemplary embodiment is a method of reducing or inhibiting the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the tumor expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor —like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74, EphA2, TROP2, B7-H3 or 5T4.

In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer, head and neck cancer or spleen cancer. In some embodiments, the tumor is a gastric cancer. In some embodiments, administration of the ADC, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

Another exemplary embodiment is a method of delaying or slowing the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the tumor expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor —like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-I, PDCDI, PDGF-R u, Prostate specific membrane antigen, PSCA (Prostate stem cell antigen precursor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74, EphA2, TROP2, B7-H3 or 5T4.

In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer or spleen cancer. In some embodiments, the tumor is a gastric cancer. In some embodiments, administration of the ADC, composition, or pharmaceutical composition delays or slows the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

In certain aspects, the present disclosure further provides methods of reducing or slowing the expansion of a cancer cell population (e.g., a CD48-expressing cancer cell population, a CD74-expressing cancer cell population, an EphA2-expressing cancer cell population, a PCAD-expressing cancer cell population, a HER2-expressing cancer cell population), comprising administering a therapeutically effective amount of an ADC or composition comprising an ADC.

An exemplary embodiment is a method of reducing or slowing the expansion of a cancer cell population in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the cancer cell population expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, EGFR, CD71, EPCAM, FOLR1, ENPP3, MET, AXL, SLC34A2, Nectin4, TROP2, LIV1, CD46, MSLN, F3, MUC16, SLC39A6, TFRC, TACSTD2, or GPNMB. In some embodiments, the target antigen is EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CEA, CEA-related antigen, ch4D5, CLDN18.2, CRIPTO (CR, CRI, CRGF, TDGF1), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor —like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), MCP-I, MDP (DPEPI), MPF, MSLN, SMR, mesothelin, megakaryocyte, PD-1, PDCDI, PDGF-R u, Prostate specific membrane antigen, PSCA (Prostate stem cell antigen precursor), PSCA hlg, RANKL, RON, SDCI, Sema Sb, STEAP 1, STEAP2, PCANAP 1, STAMP 1, STEAP2, STMP, prostate cancer associated gene 1, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-1, VEGFR-2, or Vimentin. In some embodiments, the target antigen is EGFR, CD7, HER2, EPCAM, FOLR1, ENPP3, MET, AXL, SLC34A2, Nectin4, MSLN, F3, MUC16, SLC39A6, TFRC, TACSTD2, or GPNMB. In some embodiments, the target antigen is PCAD, HER2, CD48, CD74, EphA2, TROP2, B7-H3 or 5T4. In some embodiments, the cancer cell population is from a tumor or a hematological cancer. In some embodiments, the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer or head and neck cancer. In some embodiments, the cancer cell population is from a lymphoma or gastric cancer. In some embodiments, administration of the ADC, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to the population in the absence of treatment. In some embodiments, administration of the ADC, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to expansion in the absence of treatment.

Also provided herein are methods of determining whether a subject having or suspected of having a cancer will be responsive to treatment with the disclosed ADCs and compositions. An exemplary embodiment is a method of determining whether a subject having or suspected of having a cancer will be responsive to treatment with an ADC, composition, or pharmaceutical composition (e.g., any of the exemplary ADCs, compositions, or pharmaceutical compositions disclosed herein) by providing a biological sample from the subject; contacting the sample with the ADC; and detecting binding of the ADC to cancer cells in the sample. In some embodiments, the sample is a tissue biopsy sample, a blood sample, or a bone marrow sample. In some embodiments, the method comprises providing a biological sample from the subject; contacting the sample with the ADC; and detecting one or more markers of cancer cell death in the sample (e.g., increased expression of one or more apoptotic markers, reduced expansion of a cancer cell population in culture, etc.).

Further provided herein are therapeutic uses of the disclosed ADCs and compositions. An exemplary embodiment is an ADC, composition, or pharmaceutical composition (e.g., any of the exemplary ADCs, compositions, or pharmaceutical compositions disclosed herein) for use in treating a subject having or suspected of having a cancer (e.g., a PCAD-expressing cancer, a HER2-expressing cancer, a EphA2-expressing cancer, a CD48-expressing cancer, a CD74-expressing cancer). Another exemplary embodiment is a use of an ADC, composition, or pharmaceutical composition (e.g., any of the exemplary ADCs, compositions, or pharmaceutical compositions disclosed herein) in treating a subject having or suspected of having a cancer (e.g., a PCAD-expressing cancer, a HER2-expressing cancer, a EphA2-expressing cancer, a CD48-expressing cancer, a CD74-expressing cancer). Another exemplary embodiment is a use of an ADC, composition, or pharmaceutical composition (e.g., any of the exemplary ADCs, compositions, or pharmaceutical compositions disclosed herein) in a method of manufacturing a medicament for treating a subject having or suspected of having a cancer (e.g., a PCAD-expressing cancer, a HER2-expressing cancer, a EphA2-expressing cancer, a CD48-expressing cancer, a CD74-expressing cancer). Methods for identifying subjects having cancers that express a target antigen (e.g., CD48, CD74, EphA2, PCAD, HER2, TROP2, B7-H3 or 5T4) are known in the art and may be used to identify suitable patients for treatment with a disclosed ADC compound or composition.

Moreover, ADCs of the present disclosure may be administered to a non-human mammal expressing an antigen with which the ADC is capable of binding for veterinary purposes or as an animal model of human disease. Regarding the latter, such animal models may be useful for evaluating the therapeutic efficacy of the disclosed ADCs (e.g., testing of dosages and time courses of administration).

The therapeutic compositions used in the practice of the foregoing methods may be formulated into pharmaceutical compositions comprising a pharmaceutically acceptable carrier suitable for the desired delivery method. An exemplary embodiment is a pharmaceutical composition comprising an ADC of the present disclosure and a pharmaceutically acceptable carrier, e.g., one suitable for a chosen means of administration, e.g., intravenous administration. The pharmaceutical composition may also comprise one or more additional inactive and/or therapeutic agents that are suitable for treating or preventing, for example, a cancer (e.g., a standard-of-care agent, etc.). The pharmaceutical composition may also comprise one or more carrier, excipient, and/or stabilizer components, and the like. Methods of formulating such pharmaceutical compositions and suitable formulations are known in the art (see, e.g., “Remington's Pharmaceutical Sciences,” Mack Publishing Co., Easton, PA).

Suitable carriers include any material that, when combined with the therapeutic composition, retains the anti-tumor function of the therapeutic composition and is generally non-reactive with the patient's immune system. Pharmaceutically acceptable carriers include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In many cases, isotonic agents are included, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the ADC.

A pharmaceutical composition of the present disclosure can be administered by a variety of methods known in the art. The route and/or mode of administration may vary depending upon the desired results. In some embodiments, the therapeutic formulation is solubilized and administered via any route capable of delivering the therapeutic composition to the cancer site. Potentially effective routes of administration include, but are not limited to, parenteral (e.g., intravenous, subcutaneous), intraperitoneal, intramuscular, intratumor, intradermal, intraorgan, orthotopic, and the like. In some embodiments, the administration is intravenous, subcutaneous, intraperitoneal, or intramuscular. The pharmaceutically acceptable carrier should be suitable for the route of administration, e.g., intravenous or subcutaneous administration (e.g., by injection or infusion). Depending on the route of administration, the active compound(s), i.e., the ADC and/or any additional therapeutic agent, may be coated in a material to protect the compound(s) from the action of acids and other natural conditions that may inactivate the compound(s). Administration can be either systemic or local.

The therapeutic compositions disclosed herein may be sterile and stable under the conditions of manufacture and storage, and may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The form depends on the intended mode of administration and therapeutic application. In some embodiments, the disclosed ADCs can be incorporated into a pharmaceutical composition suitable for parenteral administration. The injectable solution may be composed of either a liquid or lyophilized dosage form in a flint or amber vial, ampule, or pre-filled syringe, or other known delivery or storage device. In some embodiments, one or more of the ADCs or pharmaceutical compositions is supplied as a dry sterilized lyophilized powder or water free concentrate in a hermetically sealed container and can be reconstituted (e.g., with water or saline) to the appropriate concentration for administration to a subject.

Typically, a therapeutically effective amount or efficacious amount of a disclosed composition, e.g., a disclosed ADC, is employed in the pharmaceutical compositions of the present disclosure. The composition, e.g., one comprising an ADC, may be formulated into a pharmaceutically acceptable dosage form by conventional methods known in the art. Dosages and administration protocols for the treatment of cancers using the foregoing methods will vary with the method and the target cancer, and will generally depend on a number of other factors appreciated in the art.

Dosage regimens for compositions disclosed herein, e.g., those comprising ADCs alone or in combination with at least one additional inactive and/or active therapeutic agent, may be adjusted to provide the optimum desired response (e.g., a therapeutic response). For example, a single bolus of one or both agents may be administered at one time, several divided doses may be administered over a predetermined period of time, or the dose of one or both agents may be proportionally increased or decreased as indicated by the exigencies of the therapeutic situation. In some embodiments, treatment involves single bolus or repeated administration of the ADC preparation via an acceptable route of administration. In some embodiments, the ADC is administered to the patient daily, weekly, monthly, or any time period in between. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage values for compositions comprising an ADC and/or any additional therapeutic agent(s), may be selected based on the unique characteristics of the active compound(s), and the particular therapeutic effect to be achieved. A physician or veterinarian can start doses of the ADC employed in the pharmaceutical composition at levels lower than that required to achieve the desired therapeutic effect and gradually increase the dosage until the desired effect is achieved. In general, effective doses of the compositions of the present disclosure, for the treatment of a cancer may vary depending upon many different factors, including means of administration, target site, physiological state of the patient, whether the patient is human or an animal, other medications administered, and whether treatment is prophylactic or therapeutic. The selected dosage level may also depend upon a variety of pharmacokinetic factors including the activity of the particular compositions of the present disclosure employed, or the ester, salt, or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Treatment dosages may be titrated to optimize safety and efficacy.

Toxicity and therapeutic efficacy of compounds provided herein can be determined by standard pharmaceutical procedures in cell culture or in animal models. For example, LD50, ED50, EC50, and IC50 may be determined, and the dose ratio between toxic and therapeutic effects (LD50/ED50) may be calculated as the therapeutic index. The data obtained from in vitro and in vivo assays can be used in estimating or formulating a range of dosage for use in humans. For example, the compositions and methods disclosed herein may initially be evaluated in xenogeneic cancer models (e.g., an NCI-H929 multiple myeloma mouse model).

In some embodiments, an ADC or composition comprising an ADC is administered on a single occasion. In other embodiments, an ADC or composition comprising an ADC is administered on multiple occasions. Intervals between single dosages can be, e.g., daily, weekly, monthly, or yearly. Intervals can also be irregular, based on measuring blood levels of the administered agent (e.g., the ADC) in the patient in order to maintain a relatively consistent plasma concentration of the agent. The dosage and frequency of administration of an ADC or composition comprising an ADC may also vary depending on whether the treatment is prophylactic or therapeutic. In prophylactic applications, a relatively low dosage may be administered at relatively infrequent intervals over a long period of time. Some patients continue to receive treatment for the rest of their lives. In therapeutic applications, a relatively higher dosage at relatively shorter intervals is sometimes required until progression of the disease is reduced or terminated, and preferably until the patient shows partial or complete amelioration of one or more symptoms of disease. Thereafter, the patient may be administered a lower, e.g., prophylactic regime.

The above therapeutic approaches can be combined with any one of a wide variety of additional surgical, chemotherapy, or radiation therapy regimens. In some embodiments, the ADCs or compositions disclosed herein are co-formulated and/or co-administered with one or more additional therapeutic agents, e.g., one or more chemotherapeutic agents, one or more standard-of-care agents for the particular condition being treated.

Kits for use in the therapeutic and/or diagnostic applications described herein are also provided. Such kits may comprise a carrier, package, or container that is compartmentalized to receive one or more containers such as vials, tubes, and the like, each of the container(s) comprising one of the separate elements to be used in a method disclosed herein. A label may be present on or with the container(s) to indicate that an ADC or composition within the kit is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that an ADC or composition within the kit is used for diagnosing or treating a condition, such as a cancer a described herein.

In some embodiments, a kit comprises an ADC or composition comprising an ADC. In some embodiments, the kit further comprises one or more additional components, including but not limited to: instructions for use; other reagents, e.g., a therapeutic agent (e.g., a standard-of-care agent); devices, containers, or other materials for preparing the ADC for administration; pharmaceutically acceptable carriers; and devices, containers, or other materials for administering the ADC to a subject. Instructions for use can include guidance for therapeutic applications including suggested dosages and/or modes of administration, e.g., in a patient having or suspected of having a cancer. In some embodiments, the kit comprises an ADC and instructions for use of the ADC in treating, preventing, and/or diagnosing a cancer.

Methods of Conjugation The present invention provides various methods of conjugating Linker-Drug groups of the invention to antibodies or antibody fragments to produce Antibody Drug Conjugates which comprise a linker having one or more hydrophilic groups.

A general reaction scheme for the formation of Antibody Drug Conjugates of Formula (A-2) is shown in Scheme 1 below:

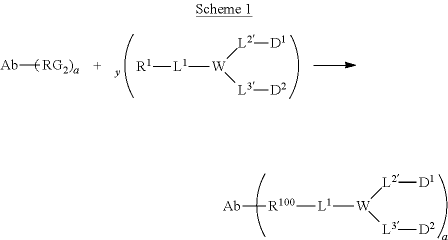

where: RG₂ is a reactive group which reacts with a compatible R¹ group to form a corresponding R¹⁰⁰ group (such groups are illustrated in Table F and Table G). D¹, D², R¹, L¹, L^{2′} L^{3′}, Ab, W, a and R¹⁰⁰ are as defined herein.

Scheme 2 further illustrates this general approach for the formation of Antibody Drug Conjugates of Formula (A-2), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 2 shows the antibody having four RG₂ groups.

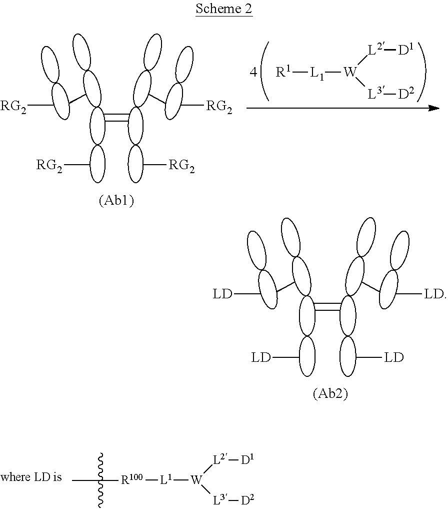

In one aspect, Linker-Drug groups are conjugated to antibodies via modified cysteine residues in the antibodies (see for example WO2014/124316). Scheme 3 illustrates this approach for the formation of Antibody Drug Conjugates of Formula (A-2) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 3 shows the antibody having four free thiol groups.

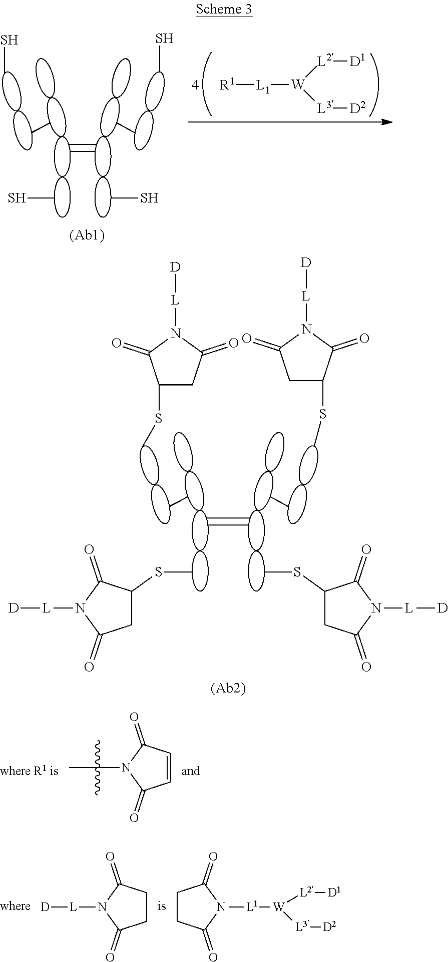

In another aspect, Linker-Drug groups are conjugated to antibodies via lysine residues in the antibodies. Scheme 4 illustrates this approach for the formation of Antibody Drug Conjugates of Formula (A-2) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 4 shows the antibody having four amine groups.

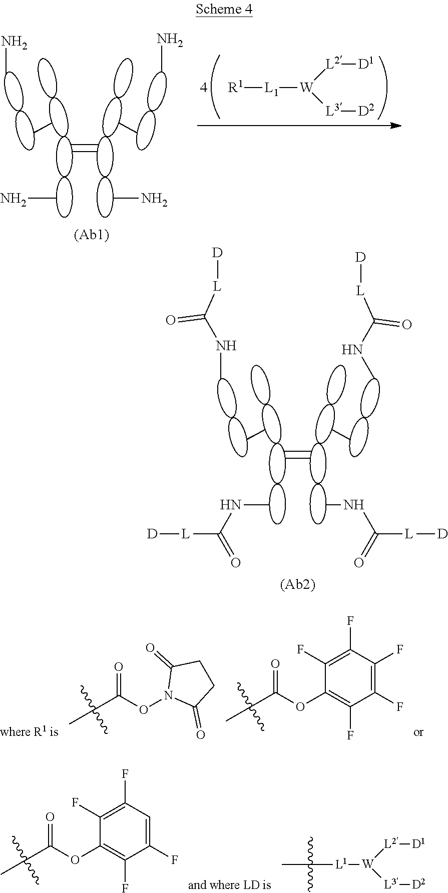

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 5 illustrates this approach for the formation of Antibody Drug Conjugates of Formula (A-2).

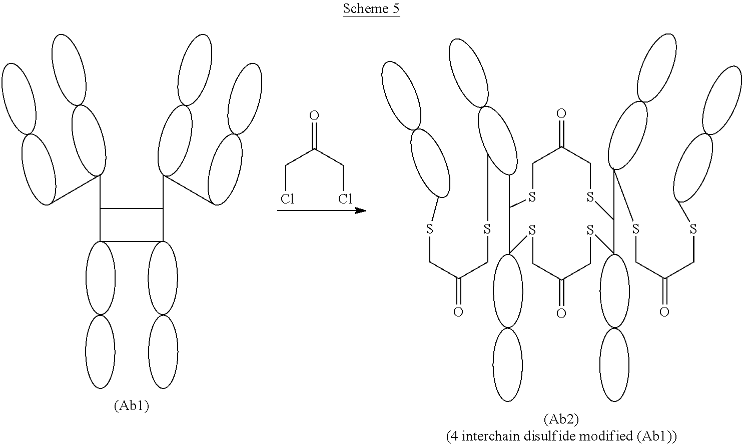

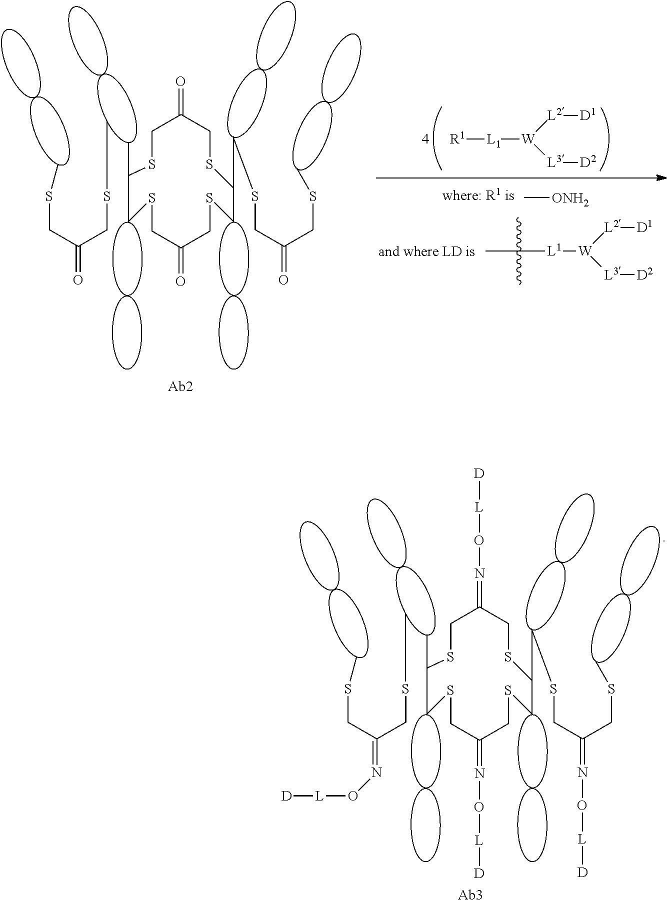

A general reaction scheme for the formation of Antibody Drug Conjugates of Formula (D3-2) is shown in Scheme 6 below:

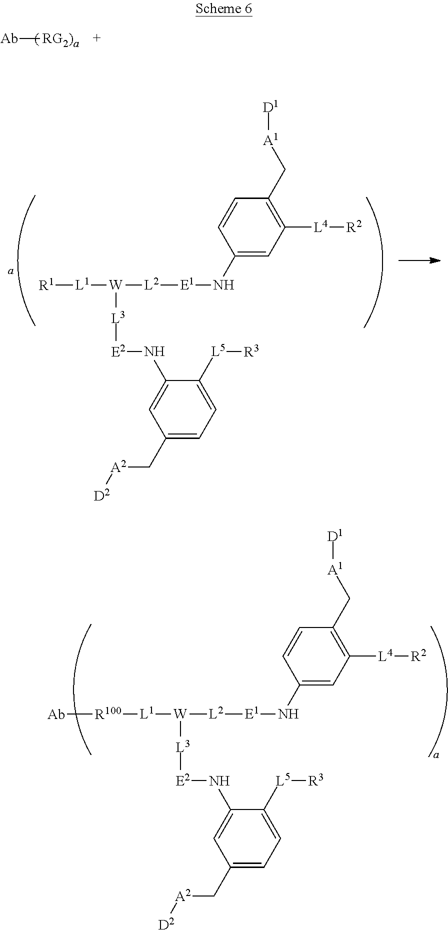

where: RG₂ is a reactive group which reacts with a compatible R¹ group to form a corresponding R¹⁰⁰ group (such groups are illustrated in Table F and Table G). D¹, D², R¹, L¹, W, L², L³, E¹, E², L⁴, L⁵, A¹, A², R², R³, Ab, a and R¹⁰⁰ are as defined herein.

Scheme 7 further illustrates this general approach for the formation of Antibody Drug Conjugates of Formula (D3-2), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 7 shows the antibody having four RG₂ groups.

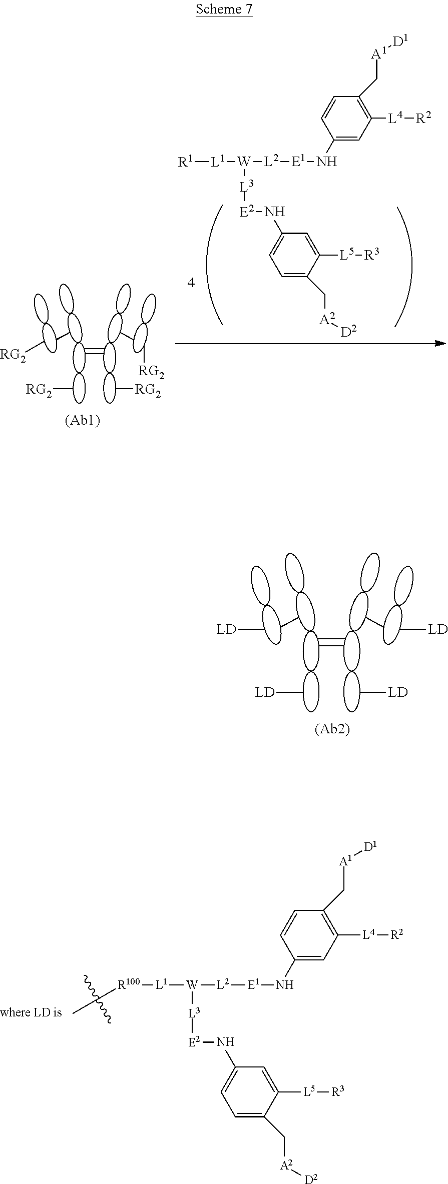

In one aspect, Linker-Drug groups are conjugated to antibodies via modified cysteine residues in the antibodies (see for example WO2014/124316). Scheme 8 illustrates this approach for the formation of Antibody Drug Conjugates of Formula (D3-2) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 8 shows the antibody having four free thiol groups.

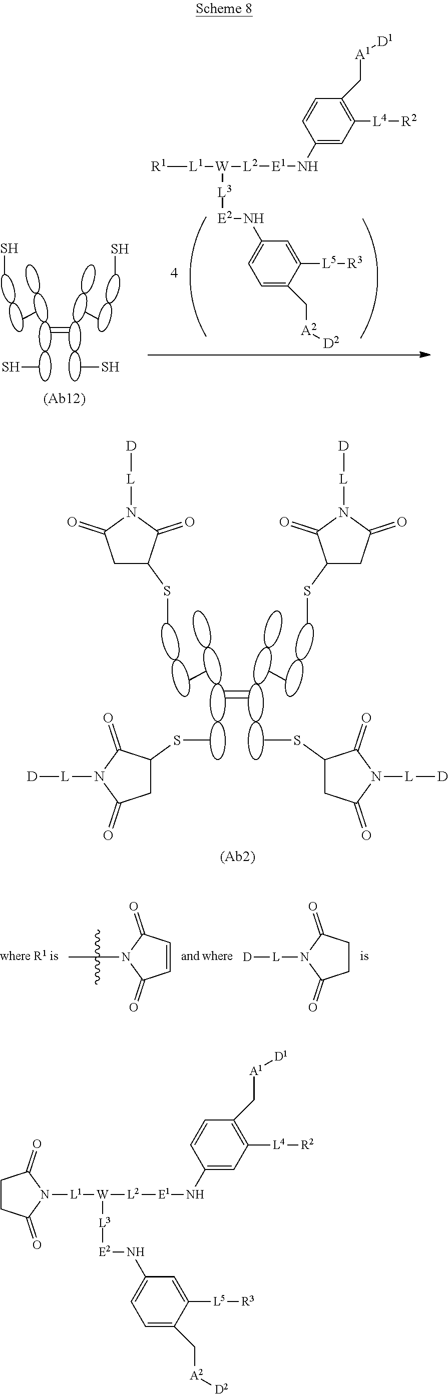

In another aspect, Linker-Drug groups are conjugated to antibodies via lysine residues in the antibodies. Scheme 9 illustrates this approach for the formation of Antibody Drug Conjugates of Formula (D3-2) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 9 shows the antibody having four amine groups.

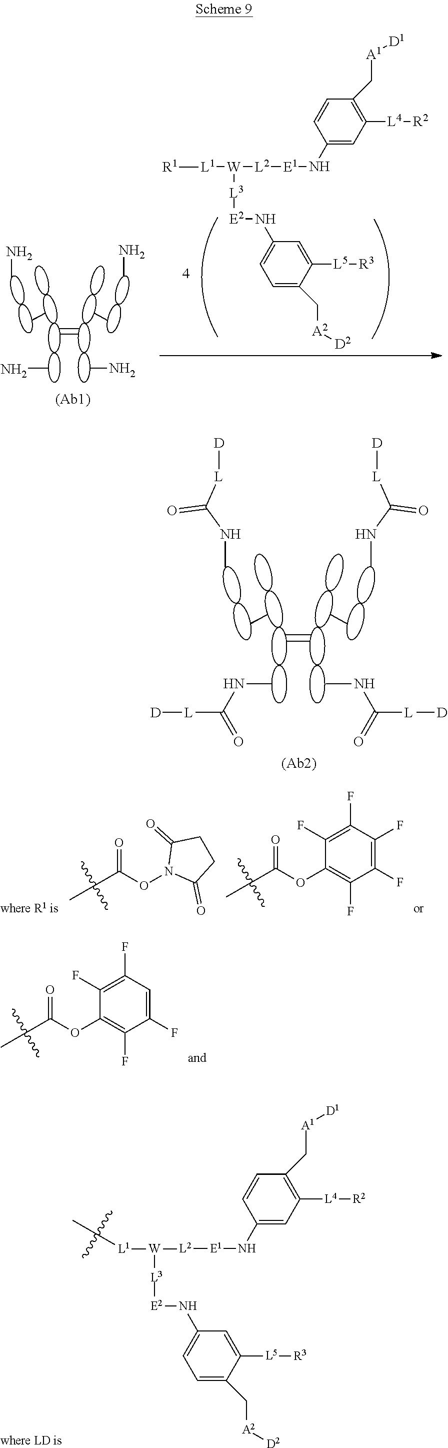

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 10 illustrates this approach for the formation of Antibody Drug Conjugates of Formula (D3-2).

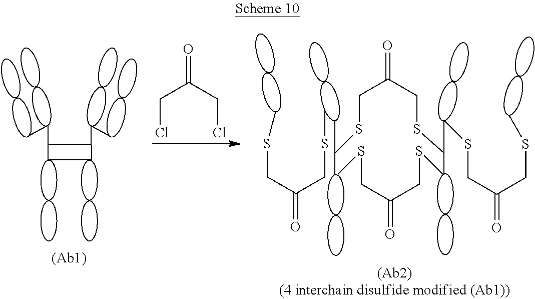

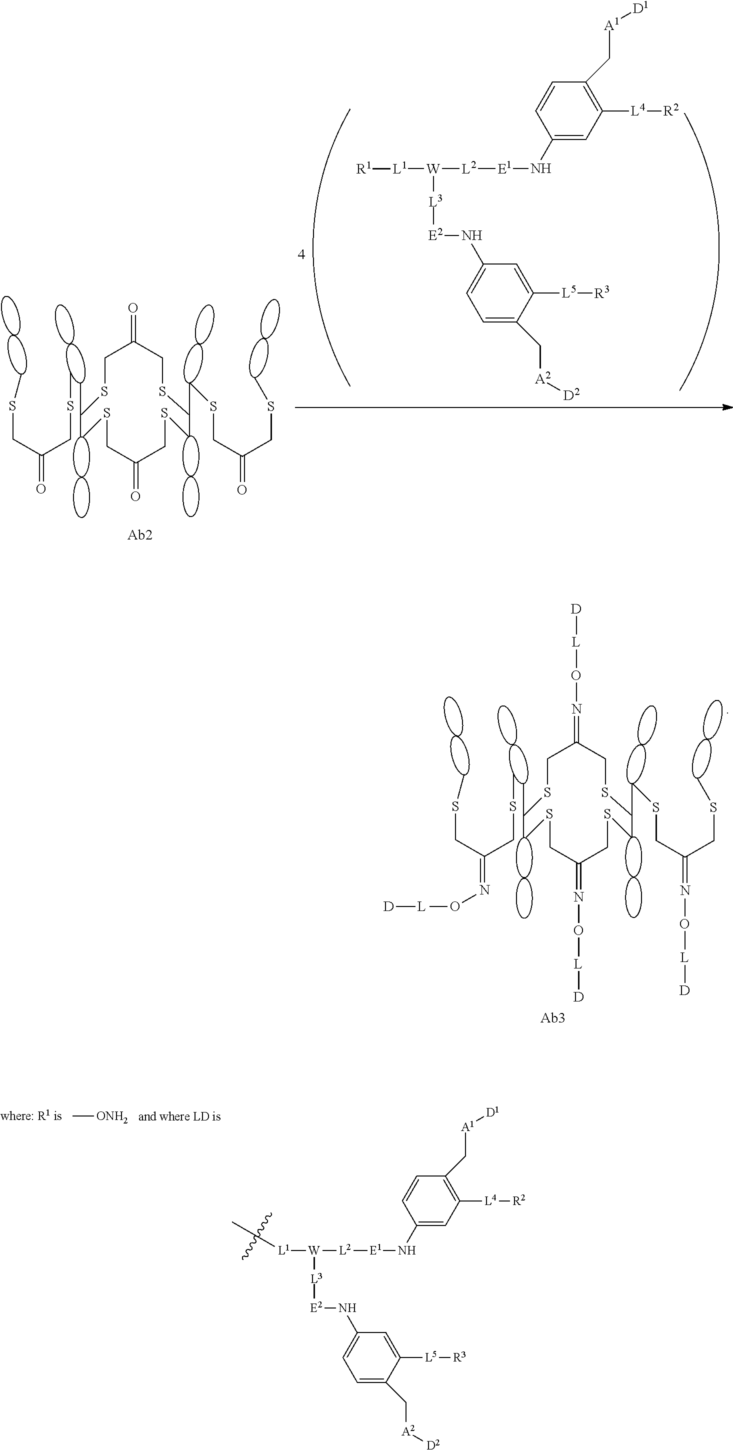

Provided are also protocols for some aspects of analytical methodology for evaluating antibody conjugates of the invention. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on other properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation over time, adherence to surfaces, hepatotoxicity, clearance rates, and pharmacokinetic exposure. See Damle, N. K., Nat Biotechnol. 2008; 26(8):884-885; Singh, S. K., Pharm Res. 2015; 32(11):3541-71. When measured by hydrophobic interaction chromatography, higher hydrophobicity index scores (i.e. elution from HIC column faster) reflect lower hydrophobicity of the conjugates. As shown in Examples below, a majority of the tested antibody conjugates showed a hydrophobicity index of greater than 0.8. In some embodiments, provided are antibody conjugates having a hydrophobicity index of 0.8 or greater, as determined by hydrophobic interaction chromatography.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example A. Synthesis and Characterization of Linkers, Linker-Payloads, and Precursors thereof

Exemplary linkers, linker-payloads, and precursors thereof were synthesized using exemplary methods described in this example. Some of the intermediates used herein, and their methods of synthesis are described in WO2020/236841.

### Abbreviations

DCC: dicyclohexylcarbodiimide
DCE: dichloroethane
DCM dichloromethane
DIEA/DIPEA: N,N-Diisopropylethylamine
DMTMM: 4-(4,6-dimethoxy-1,3,5-triazin-2-yl)-4-methyl-morpholinium chloride
DMF: dimethylformamide
DMSO: dimethylsulfoxyde
EDC/EDC-HCl: 1-Ethyl-3-(3-dimethylaminopropyl)carbodiimide hydrochloride
HATU: 1-[Bis(dimethylamino)methylene]-1H-1,2,3-triazolo[4,5-b]pyridinium 3-oxide hexafluorophosphate
HBTU: (2-(1H-benzotriazol-1-yl)-1,1,3,3-tetramethyluronium hexafluorophosphate
HOAt: 1-Hydroxy-7-azabenzotriazole
MeCN: acetonitrile
MeOH: methanol
NMP: N-methylpyrrolidine
TES: triethylsilane
TBAF: tetrabutyl ammonium fluoride
TBAI: tetrabutyl ammonium iodide
TBTU: [Bis(dimethylamino)methylene]-1H-benzotriazolium 3-Oxide Tetrafluoroborate
pTsOH: para-toluene sulfonic acid
PyBOP: benzotriazol-1-yloxytripyrrolidinophosphonium hexafluorophosphate
TSTU: O—(N-Succinimidyl)-N,N,N′,N′-tetramethyluronium tetrafluoroborate
THF: tetrahydrofuran
TFA: trifluoroacetic acid
TFE: 2,2,2-Trifluoroethanol

### Materials, Methods & General Procedures

All reagents obtained from commercial sources were used without further purification. Anhydrous solvents were obtained from commercial sources and used without further drying. Flash chromatography was performed on CombiFlash Rf (Teledyne ISCO) with pre-packed silica-gel cartridges (Macherey-Nagel Chromabond Flash). Thin layer chromatography was conducted with 5×10 cm plates coated with Merck Type 60 F254 silica-gel. Microwave heating was performed in CEM Discover® instrument.

NMR data were acquired at a temperature of 298K on a Bruker Avance NMR spectrometer equipped with a 5 mm BBFO CryoProbe with z-gradient operating at a frequency of 400.13 MHz for ¹H, 376.50 MHz for ¹⁹F, 100.61 MHz for ¹³C. Chemical shifts for the ¹H and ¹³C spectra were referenced by setting internal tetramethylsilane (TMS) to 0 ppm.

### Analytical Methods

LC/MS data was acquired using an instrument with the following parameters:

| | |
|---|---|
| Pump | Waters AcQuity UPLC Binary Solvent |
| | Manager |
| Sample Manager | Waters AcQuity UPLC Sample Manager |
| Column | Waters AcQuity UPLC Column Manager |
| Compartment | |
| Detector | Waters AcQuity UPLC PDA |
| ELSD | Shimadzu ELSD-LTII |
| Mass Spec | Waters SQD |
| Columns | AcQuity UPLC BEH C18 1.7 μm 2.1 × 50 mm |
| Eluent A1 | 0.1% Formic Acid in Water |
| Eluent B1 | 0.1% Formic Acid in Acetonitrile |
| Eluent A2 | 5 mM Ammonium Hydroxide in Water |
| Eluent B2 | 5 mM Ammonium Hydroxide in Acetonitrile |

The methods used to generate LC/MS data were as follows:

2 min acidic method:

| Eluent A1 | 0.1% Formic Acid in Water | | |
|---|---|---|---|
| Eluent B1 | 0.1% Formic Acid in Acetonitrile | | |
| Flow | 1.0 mL/min | | |
| Stop Time | 3.00 min | | |
| pH | 2.6 | | |
| | Time | % A (Eluent A1) | % B (Eluent B1) |
| Gradient | 0 | 95 | 5 |
| | 0.2 | 95 | 5 |
| | 2 | 5 | 95 |
| | 2.5 | 5 | 95 |
| | 2.6 | 95 | 5 |
| | 3 | 95 | 5 |

| Column | AcQuity UPLC BEH C18 1.7 μm 2.1 × 50 mm |
|---|---|
| Column | 50° C. |
| Temperature | |
| TAC | 210-400 nm |
| Mass Range | 120-1500 Da |
| Scan Time | 0.3 sec |

2 min basic method:

| Eluent A1 | 5 mM Ammonium Hydroxide in Water | | |
|---|---|---|---|
| Eluent B1 | 5 mM Ammonium Hydroxide in Acetonitrile | | |
| Flow | 1.0 mL/min | | |
| Stop Time | 3.00 min | | |
| pH | 10.2 | | |
| | Time | % A (Eluent A1) | % B (Eluent B1) |
| Gradient | 0 | 95 | 5 |
| | 0.2 | 95 | 5 |
| | 2 | 5 | 95 |
| | 2.5 | 5 | 95 |
| | 2.6 | 95 | 5 |
| | 3 | 95 | 5 |
| Column | AcQuity UPLC BEH C18 1.7 μm 2.1 × 50 mm | | |
| Column | 50° C. | | |
| Temperature | | | |

| | |
|---|---|
| TAC | 210-400 nm |
| Mass Range | 120-1500 Da |
| Scan Time | 0.3 sec |

5 min acidic method

| Flow | 1.0 | mL/min | | |
|---|---|---|---|---|
| Stop Time | 5.20 | min | | |
| pH | 2.6 | | | |
| | Time | | % A (Eluent A1) | % B (Eluent B1) |
| Gradient | 0 | | 98 | 2 |
| | 4.4 | | 2 | 98 |
| | 5.15 | | 2 | 98 |
| | 5.19 | | 98 | 2 |
| Column | AcQuity UPLC BEH C18 1.7 μm 2.1 × 50 mm | | | |
| Column | 50° | C. | | |
| Temperature | | | | |
| TAC | 210-400 | nm | | |
| Mass Range | 120-1500 | Da | | |
| Scan Time | 0.3 | sec | | |

HRMS data was acquired using an instrument with the following parameters:

### HRMS_QT101

| | |
|---|---|
| Pump | Waters AcQuity UPLC Binary Solvent |
| | Manager |
| Sample Manager | Waters AcQuity UPLC Sample Manager |
| Column | Waters AcQuity UPLC Column Manager |
| Compartment | |
| Detector | Waters AcQuity UPLC PDA |
| ELSD | n/a |
| Mass Spec | Waters Xevo G2 Qtof |
| Columns | AcQuity UPLC PrST C4 300 Å 1.7 μm |
| | 2.1 × 100 mm |
| | AcQuity UPLC CSH C18 1.7 μm 2.1 × 50 mm |
| | ProSwift RP-3U 4.6 × 50 mm SS |
| Eluent A1 | 0.1% Formic Acid in Water |
| Eluent B1 | 0.1% Formic Acid in Acetonitrile |
| Eluent A2 | 0.05% Triflouroacetic Acid in Water |
| Eluent B2 | 0.05% Triflouroacetic Acid in Acetonitrile |

### HRMS_QT02

| | |
|---|---|
| Pump | Waters AcQuity UPLC Binary Solvent |
| | Manager |
| Sample Manager | Waters AcQuity UPLC Sample Manager |
| Column | Waters AcQuity UPLC Column Manager |
| Compartment | |
| Detector | Waters AcQuity UPLC PDA |
| ELSD | n/a |
| Mass Spec | Waters Xevo G2 Qtof |
| Columns | AcQuity UPLC PrST C4 300 Å 1.7 μm |
| | 2.1 × 100 mm |
| | AcQuity UPLC CSH C18 1.7 μm 2.1 × 50 mm |
| | POROS R1 10 μm 2.1 × 100 mm |
| Eluent A1 | 0.1% Formic Acid in Water |
| Eluent B1 | 0.1% Formic Acid in 80:20 |
| | Isopropanol:Acetonitrile |
| Eluent A2 | 0.05% Triflouroacetic Acid in Water |
| Eluent B2 | 0.05% Triflouroacetic Acid in Acetonitrile |

The method used to generate HRMS data for linker/payloads and synthetic intermediates was as follows:

Peptide_300-4000_Da_5 min QT1

| Flow | 1.0 mL/min | | |
|---|---|---|---|
| Stop Time | 5.2 min | | |
| pH | 2.6 | | |
| | Time | % A (Eluent | % B (Eluent B2) |
| Gradient | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 1.7 μm 2.1 × 50 mm |
| Column Temp | 50° C. |
| TAC | 210-400 nm |
| Mass Range | 300-4000 Da |
| Processing | n/a |
| Scan Time | 0.5 sec |

Peptide_300-10000_Da_5 min QT1

| Flow | 1.0 mL/min | | |
|---|---|---|---|
| Stop Time | 5.2 min | | |
| pH | 2.6 | | |
| | Time | % A (Eluent A2) | % B (Eluent B2) |
| Gradient | 0 | 98 | 2 |
| | 4.4 | 2 | 98 |
| | 5.15 | 2 | 98 |
| | 5.19 | 98 | 2 |
| Column | AcQuity UPLC BEH C18 1.7 μm 2.1 × 50 mm | | |
| Column Temp | 50° C. | | |
| TAC | 210-400 nm | | |
| Mass Range | 300-4000 Da | | |
| Processing | 300-10000 Da | | |
| Range | | | |

| | |
|---|---|
| Scan Time | 0.5 sec |

Peptide_300-4000_Da_5 min QT2

| Flow | 1.0 mL/min | | |
|---|---|---|---|
| Stop Time | 5.2 min | | |
| pH | 2.6 | | |
| | Time | % A (Eluent A2 | % B (Eluent B2) |
| Gradient | 0 | 98 | 2 |
| | 4.4 | 2 | 98 |
| | 5.15 | 2 | 98 |
| | 5.19 | 98 | 2 |
| Column | AcQuity UPLC BEH C18 1.7 μm 2.1 × 50 mm | | |
| Column Temp | 80° C. | | |
| TAC | 210-400 nm | | |
| Mass Range | 300-4000 Da | | |
| Processing | n/a | | |
| Range | | | |

| | |
|---|---|
| Scan Time | 0.2 sec |

Peptide_300-10000_Da_5 min QT2

| Flow | 1.0 mL/min | | |
|---|---|---|---|
| Stop Time | 5.2 min | | |
| pH | 2.6 | | |
| | Time | % A (Eluent | % B (Eluent B2) |
| Gradient | 0 | 98 | 2 |
| | 4.4 | 2 | 98 |
| | 5.15 | 2 | 98 |
| | 5.19 | 98 | 2 |
| Column | AcQuity UPLC BEH C18 1.7 μm 2.1 × 50 mm | | |
| Column Temp | 80° C. | | |
| TAC | 210-400 nm | | |
| Mass Range | 300-4000 Da | | |
| Processing | 300-10000 Da | | |

| | |
|---|---|
| Scan Time | 0.2 sec |

### Preparative RP-HPLC:

Preparative-HPLC (“Prep-HPLC”) data were acquired using Teledyne ISCO purification systems using C18 or C4 RP ISCO or ISCO-gold columns.

Four Prep-HPLC methods were used:
a. TFA method: solvent: A water+0.05% TFA, B acetonitrile+0.05% TFA, gradient from 5 to 100% B in 15 to 30 CV
b. NH₄HCO₃ method: solvent: A water+0.02 M NH₄HCO₃, B acetonitrile/water 80/20+0.02 M NH₄HCO₃, gradient from 5 to 100% B in 15 to 30 CV
c. Neutral method: solvent: A water, B acetonitrile, gradient from 5 to 100% B in 15 to 30 CV
d. Formic Acid method: solvent: A water+0.05% Formic Acid, B acetonitrile+0.05% Formic Acid, gradient from 5 to 100% B in 15 to 30 CV
   Gradient variations of methods a.-d. were employed as appropriate. All the fractions containing the pure compound were combined and directly freeze-dried to afford the compound as an amorphous powder.

### Synthesis of 2-(bromomethyl)-4-nitrobenzoic acid

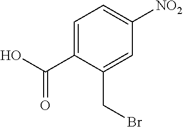 [SMILES: O=C(O)c1ccc([N+](=O)[O-])cc1CBr]

To a stirred solution of 2-methyl-4-nitrobenzoic acid (300 g, 1.5371 mol) in CCl₄ (3000 mL) was added NBS (300.93 g, 1.6908 mol) and AIBN (37.86 g, 0.2305 mol) at rt. The reaction mixture was stirred at 80° C. for 16 h. Reaction mixture was monitored by TLC analysis. The reaction mixture was diluted with sat. NaHCO₃ solution (2 lit) and extracted with ethyl acetate (2×2 lit). The combined organic layer was dried over anhydrous sodium sulphate and concentrated under reduced pressure. The crude compound was purified by column chromatography on silica gel using 2-3% of ethylacetate in petroleum ether as an eluent and 2-(bromomethyl)-4-nitrobenzoic acid was obtained (250 g, 59% yield). ¹H NMR (400 MHz, CDCl3): δ 8.35 (d, J=2.0 Hz, 1H), 8.20 (q, J=8.8, 2.4 Hz, 1H), 8.12 (d, J=8.8 Hz, 1H), 4.97 (s, 2H), 4.00 (s, 3H).

### Synthesis of 4-nitro-2-((prop-2-yn-1-yloxy)methyl)benzoic acid

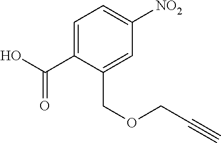 [SMILES: C#CCOCc1cc([N+](=O)[O-])ccc1C(=O)O]

To the mixture of 2-(bromomethyl)-4-nitrobenzoic acid (250 g, 0.9122 mol) in ACN (5000 mL) was added prop-2-yn-1-ol (255.68 g, 265.50 mL, 4.5609 mol, d=0.963 g/mL) and Cs₂CO₃ (743.03 g, 2.2805 mol) at rt. The resulting mixture was heated to 80° C. for 16 h. The reaction mixture was filtered through celite pad washed with ethylacetate (2 lit). The filterate was concentrated under reduced pressure. The obtained crude compound was added sat. NaHCO₃ solution (1 lit) and the aq layer was acidified to pH 2 by using 2N HCl (2 lit). After filtration vacuum drying 4-nitro-2-((prop-2-yn-1-yloxy)methyl)benzoic acid was obtained (130 g, 60.6%). ¹H NMR (400 MHz, DMSO): δ 13.61 (brs, 1H), 8.37 (d, J=2.4 Hz, 1H), 8.23 (dd, J=2.4, 8.4 Hz, 1H), 8.10 (d, J=8.8 Hz, 1H), 4.95 (s, 2H), 4.37 (d, J=2.4 Hz, 2H), 3.52 (t, J=2.4 Hz, 1H) Synthesis of methyl 4-nitro-2-((prop-2-yn-1-yloxy)methyl)benzoate

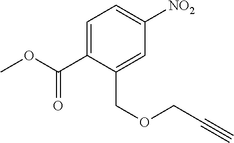 [SMILES: C#CCOCc1cc([N+](=O)[O-])ccc1C(=O)OC]

To a stirred solution of 4-nitro-2-((prop-2-yn-1-yloxy)methyl)benzoic acid (130 g, 0.5527 mol) in MeOH (1300 mL) was added SOCl₂ (526.08 g, 320.78 mL, 4.4219 mol, d=1.64 g/mL) slowly at 0° C. The reaction stirred at 70° C. for 4 h. The reaction solvent was evaporated under reduced pressure. The obtained residue was dissolved in ethylacetate (1000 mL) and washed with sat.NaHCO₃ (600 mL), water (500 mL) and brine solution (500 mL). The separated organic layer was dried over sodium sulphate, filtered and evaporated under reduced pressure to yield methyl 4-nitro-2-((prop-2-yn-1-yloxy)methyl)benzoate (110 g, 80% yield). ¹H NMR (400 MHz, CDCl3): δ 8.56 (t, J=0.8 Hz, 1H), 8.18-8.09 (m, 2H), 5.03 (s, 2H), 4.35 (d, J=2.4 Hz, 2H), 3.96 (s, 3H), 2.49 (t, J=2.4 Hz, 1H).

### Synthesis of methyl 4-amino-2-((prop-2-yn-1-yloxy)methyl)benzoate

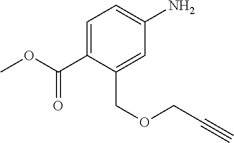 [SMILES: C#CCOCc1cc(N)ccc1C(=O)OC]

To a solution of methyl 4-nitro-2-((prop-2-yn-1-yloxy)methyl)benzoate (110 g, 0.4414 mol) in a mixture of EtOH (1100 mL) and H₂O (550 mL) was added Fe Powder (197.21 g, 3.5310 mol) and NH₄Cl (188.88 g, 3.5310 mol) at rt. The resulting mixture was heated at 80° C. for 16 h. The reaction mixture was cooled to rt and filtered through celite and washed with ethylacetate (2 lit). The filtrate was concentrated under reduced pressure up to half of the volume. To the residue ethylacetate (1.5 lit) was added and separated the two layers and the aqueous layer was extracted with ethyl acetate (2 lit). The combined organic layer was dried over anhydrous sodium sulphate and concentrated under reduced pressure to obtain crude product. Purification by SiO₂ column chromatography (15-20% of ethylacetate in pet-ether) yielded methyl 4-amino-2-((prop-2-yn-1-yloxy)methyl)benzoate (70 g, 72% yield). ¹H NMR (400 MHz, CDCl3): δ 7.67 (d, J=8.8 Hz, 1H), 6.78 (t, J=1.6 Hz, 1H), 6.48 (q, J=8.4, 2.4 Hz, 1H), 4.79 (s, 2H), 4.25 (d, J=2.4 Hz, 2H), 3.70 (d, J=4.0 Hz, 3H), 3.42 (t, J=2.4 Hz, 1H).

### Synthesis of (4-amino-2-((prop-2-yn-1-yloxy)methyl)phenyl)methanol

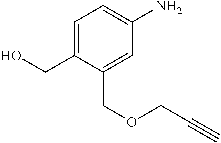

To a stirred solution of THF (1000 mL) was added LiAlH₄ (1 M in THF) (21.23 g, 798.2 mmol, 798.2 mL) slowly at 0° C. A solution of methyl 4-amino-2-((prop-2-yn-1-yloxy)methyl)benzoate (70 g, 319.3 mmol) in THF (800 mL) was added slowly at 0° C. The reaction was stirred at rt for 4 h. The reaction mixture was cooled to 0° C., then was added water (22 mL) very slowly and followed by the addition of 20% NaOH (22 mL) and water (66 mL). The reaction mixture was stirred at 0° C. for 30 min. Anhydrous sodium sulphate was added to absorb excess of water. The mixture was filtered through celite. The filter cake was washed with ethylacetate (1000 mL) and 10% MeOH/DCM (500 mL). The filtrate was concentrated under reduced pressure. The resulting crude compound was purified by SiO₂ column chromatography (35-40% of ethylacetate in pet-ether as an eluent) to give yield (4-amino-2-((prop-2-yn-1-yloxy)methyl)phenyl)methanol (50.6 g, 83% yield). ¹H NMR (400 MHz, CDCl3): δ 6.98 (d, J=8.0 Hz, 1H), 6.56 (d, J=2.4 Hz, 1H), 6.43 (dd, J=2.4, 8.0 Hz, 1H), 4.98 (s, 2H), 4.64 (t, J=5.2 Hz, 1H), 4.47 (s, 2H), 4.34 (d, J=5.6 Hz, 2H), 4.15 (d, J=2.4 Hz, 2H), 3.46 (t, J=2.4 Hz, 1H).

### Synthesis of (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate

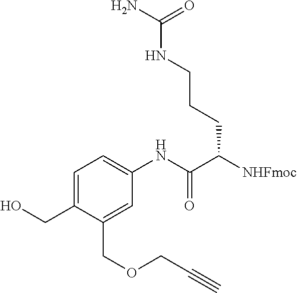

To a solution of (4-amino-2-((prop-2-yn-1-yloxy)methyl)phenyl)methanol (1.92 g, 10.04 mmol, 1.0 equiv.), (9H-fluoren-9-yl)methyl (S)-(1-amino-1-oxo-5-ureidopentan-2-yl)carbamate (3.99 g, 10.04 mmol, 1.0 equiv.), and (1-[bis(dimethylamino)methylene]-1H-1,2,3-triazolo[4,5-b]pyridinium 3-oxid hexafluorophosphate (4.20 g, 11.04 mmol, 1.1 equiv.) in DMF (10 mL) was added N,N-diisopropylethylamine (2.62 mL, 15.06 mmol, 1.5 equiv.). After stirring at ambient temperature for 1 hour, the mixture was poured into water (200 mL). The resulting solids were filtered, rinsed with water, and dried under vacuum, and (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate was obtained (6.08 g, 99%). LCMS: MH+=571.5; Rt=0.93 min (2 min acidic method-Method A).

### Synthesis of (S)-2-amino-N-(4-(hydroxymethyl)-3-((prop-2-yn-1-yloxy)methyl)phenyl)-5-ureidopentanamide

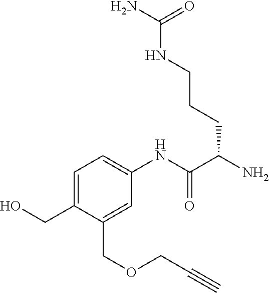 [SMILES: C#CCOCc1cc(NC(=O)[C@@H](N)CCCNC(N)=O)ccc1CO]

To (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate (6.08 g, 10.65 mmol, 1.0 equiv.) was added dimethylamine (2 M in THF, 21.31 mL, 42.62 mmol, 4 equiv.). After stirring at ambient temperature for 1.5 hours, the supernatant solution was decanted from the gum-like residue that had formed. The residue was triturated with ether (3×50 mL) and the resulting solids were filtered, washed with ether, and dried under vacuum. (S)-2-amino-N-(4-(hydroxymethyl)-3-((prop-2-yn-1-yloxy)methyl)phenyl)-5-ureidopentanamide was obtained (3.50 g, 10.04 mmol, 94%). LCMS: MH+349.3; Rt=0.42 min (2 min acidic method-Method A).

### Synthesis of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate

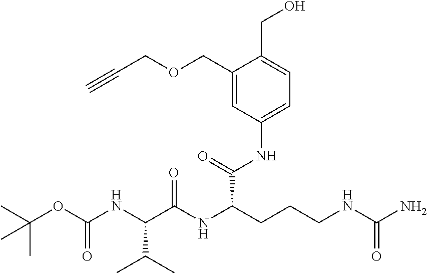 [SMILES: C#CCOCc1cc(NC(=O)[C@H](CCCNC(N)=O)NC(=O)[C@@H](NC(=O)OC(C)(C)C)C(C)C)ccc1CO]

To a solution of (S)-2-amino-N-(4-(hydroxymethyl)-3-((prop-2-yn-1-yloxy)methyl)phenyl)-5-ureidopentanamide (3.50 g, 10.04 mmol, 1.0 equiv.), (tert-butoxycarbonyl)-L-valine (2.62 g, 12.05 mmol, 1.2 equiv.), and (1-[bis(dimethylamino)methylene]-1H-1,2,3-triazolo[4,5-b]pyridinium 3-oxid hexafluorophosphate (4.58 g, 12.05 mmol, 1.2 equiv.) in DMF (10 mL) was added N,N-diisopropylethylamine (3.50 mL, 20.08 mmol, 2.0 equiv). After stirring at ambient temperature for 2 hours, the mixture was poured into water (200 mL) and the resulting suspension was extracted with EtOAc (3×100 mL). The combined organic layers were dried over sodium sulfate and concentrated under vacuum. After purification by ISCO SiO₂ chromatography (0-20% methanol/dichloromethane), tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate was obtained (2.49 g, 4.55 mmol, 45%). 1H NMR (400 MHz, DMSO-d6) δ 10.00 (s, 1H), 7.96 (d, J=7.7 Hz, 1H), 7.55 (dq, J=4.9, 2.2 Hz, 2H, aryl), 7.32 (d, J=8.9 Hz, 1H, aryl), 6.76 (d, J=8.9 Hz, 1H), 5.95 (t, J=5.8 Hz, 1H), 5.38 (s, 2H), 5.01 (t, J=5.5 Hz, 1H), 4.54 (s, 2H), 4.45 (dd, J=25.2, 5.3 Hz, 3H), 4.20 (d, J=2.4 Hz, 2H), 3.83 (dd, J=8.9, 6.7 Hz, 1H), 3.49 (t, J=2.4 Hz, 1H), 2.97 (dh, J=26.0, 6.5 Hz, 2H), 1.96 (h, J=6.6 Hz, 1H), 1.74-1.50 (m, 2H), 1.39 (m, 11H), 0.84 (dd, J=16.2, 6.7 Hz, 6H). LCMS: MNa+570.5; Rt=0.79 min (2 min acidic method-Method A).

### Synthesis of 2-(((tert-butyldiphenylsilyl)oxy)methyl)-5-nitrobenzoic acid

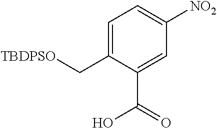

To a solution of 6-nitroisobenzofuran-1(3H)-one (90 g, 502.43 mmol, 1.00 eq) in MeOH (1000 mL) and KOH (28.19 g, 502.43 mmol, 1.00 eq) in H2O (150 mL) was added. The brown mixture was stirred at 25° C. for 1.5 h. The brown mixture was concentrated under reduced pressure to give a residue and dissolved in DCM (2000 mL). The mixture was added TBDPSCI (296.91 g, 1.08 mol, 277.49 mL, 2.15 eq) and imidazole (171.03 g, 2.51 mol, 5.00 eq) stirred at 25° C. for 12 h. The mixture was concentrated under reduced pressure to give a residue. The residue was purified by silica gel chromatography (Petroleum ether/Ethyl acetate=1/0, 1/1) and 2-(((tert-butyldiphenylsilyl)oxy)methyl)-5-nitrobenzoic acid (34 g, 74.16 mmol, 14.76% yield) was obtained as a white solid. ¹H NMR (400 MHz, METHANOL-d4) δ ppm 1.13 (s, 9H) 5.26 (s, 2H) 7.34-7.48 (m, 6H) 7.68 (br d, J=8 Hz, 4H) 8.24 (br d, J=8 Hz, 1H) 8.46 (br d, J=8 Hz, 1H) 8.74 (s, 1H)

### Synthesis of (2-(((tert-butyldiphenylsilyl)oxy)methyl)-5-nitrophenyl)methanol

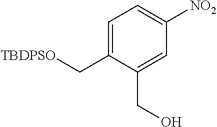

To a mixture of 2-(((tert-butyldiphenylsilyl)oxy)methyl)-5-nitrobenzoic acid (41 g, 94.14 mmol, 1 eq) in THF (205 mL) was added BH₃·THF (1 M, 470.68 mL, 5 eq). The yellow mixture was stirred at 60° C. for 2 h. The mixture was added MeOH (400 mL), and concentrated under reduced pressure to give a residue. then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (Petroleum ether/Ethyl acetate=1/0, 1/1). (2-(((tert-butyldiphenylsilyl)oxy)methyl)-5-nitrophenyl)methanol (34 g, 80.65 mmol, 85.7% yield) was obtained as a white solid.

1H NMR (400 MHz, METHANOL-d4) δ ppm 1.10 (s, 9H) 4.58 (s, 2H) 4.89 (s, 2H) 7.32-7.51 (m, 6H) 7.68 (dd, J=8, 1.38 Hz, 4H) 7.76 (d, J=8 Hz, 1H) 8.15 (dd, J=8, 2.26 Hz, 1H) 8.30 (d, J=2 Hz, 1H).

### Synthesis of 2-(((tert-butyldiphenylsilyl)oxy)methyl)-5-nitrobenzaldehyde

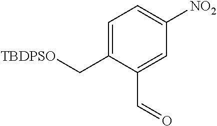

To a solution of (2-(((tert-butyldiphenylsilyl)oxy)methyl)-5-nitrophenyl)methanol (34 g, 80.65 mmol, 1 eq) in DCM (450 mL) was added MnO2 (56.09 g, 645.22 mmol, 8 eq). The black mixture was stirred at 25° C. for 36 h. The mixture was added MeOH (400 mL), and concentrated under reduced pressure to give a residue. then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (CH₂Cl₂=100%). 2-(((tert-butyldiphenylsilyl)oxy)methyl)-5-nitrobenzaldehyde (30 g, 71.51 mmol, 88.7% yield) was obtained as a white solid.

¹H NMR (400 MHz, CHLOROFORM-d) δ ppm 1.14 (s, 9H) 5.26 (s, 2H) 7.34-7.53 (m, 6H) 7.60-7.73 (m, 4H) 8.13 (d, J=8 Hz, 1H) 8.48 (dd, J=8, 2.51 Hz, 1H) 8.67 (d, J=2 Hz, 1H) 10.16 (s, 1H).

### Synthesis of N-(2-(((tert-butyldiphenylsilyl)oxy)methyl)-5-nitrobenzyl)prop-2-yn-1-amine

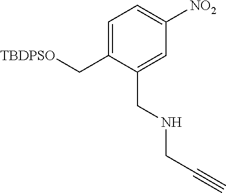

To a solution of 2-(((tert-butyldiphenylsilyl)oxy)methyl)-5-nitrobenzaldehyde (12.6 g, 30.03 mmol, 1 eq) in DCM (130 mL) was added prop-2-yn-1-amine (4.14 g, 75.08 mmol, 4.81 mL, 2.5 eq) and MgSO₄ (36.15 g, 300.33 mmol, 10 eq) then the suspension mixture was stirred at 25° C. for 24 hr. Take a little reaction solution and treat with NaBH₄ the TLC showed one new point was formed. The reaction mixture was filtered and concentrated under reduced pressure to give a residue. (E)-N-[[2-[[tert-butyl(diphenyl)silyl]oxymethyl]-5-nitro-phenyl]methyl]prop-2-yn-1-imine (12 g, crude) was obtained as a yellow solid. ¹H NMR (400 MHz, CHLOROFORM-d) δ ppm 1.11 (s, 9H) 2.48 (t, J=2.38 Hz, 1H) 4.52 (t, J=2.13 Hz, 2H) 5.09 (s, 2H) 7.35-7.49 (m, 6H) 7.63-7.72 (m, 4H) 7.79 (d, J=8.53 Hz, 1H) 8.25 (dd, J=8.53, 2.51 Hz, 1H) 8.68 (d, J=2.26 Hz, 1H) 8.84 (t, J=1.88 Hz, 1H).

(E)-N-[[2-[[tert-butyl(diphenyl)silyl]oxymethyl]-5-nitro-phenyl]methyl]prop-2-yn-1-imine (12 g, 26.28 mmol, 1 eq) was dissolved in MeOH (100 mL) and THF (50 mL), then NaBH₄ (1.49 g, 39.42 mmol, 1.5 eq) was added and the yellow mixture was stirred at −20° C. for 2 hr. LCMS showed that the desired compound was detected. The reaction mixture was quenched by addition MeOH 200 mL at −20° C., and then concentrated under reduced pressure to give a residue. The residue was dissolved with EtOAc 500 mL washed with brine 150 mL, dried over anhydrous Na₂SO₄, filtered and concentrated under reduced pressure to give a residue. The residue was purified by flash silica gel chromatography (Eluent of 0-10% Ethyl acetate/Petroleum ether gradient). N-(2-(((tert-butyldiphenylsilyl)oxy)methyl)-5-nitrobenzyl)prop-2-yn-1-amine (9 g, 18.45 mmol, 70% yield) was obtained as a pale yellow oil. ¹H NMR (400 MHz, CHLOROFORM-d) δ ppm 1.12 (s, 9H) 2.13 (t, J=2.38 Hz, 1H) 3.33 (d, J=2.51 Hz, 2H) 3.80 (s, 2H) 4.93 (s, 2H) 7.36-7.49 (m, 6H) 7.69 (dd, J=7.91, 1.38 Hz, 4H) 7.77 (d, J=8.53 Hz, 1H) 8.16 (dd, J=8.41, 2.38 Hz, 1H) 8.24 (d, J=2.26 Hz, 1H).

### Synthesis of (9H-fluoren-9-yl)methyl (2-(((tert-butyldiphenylsilyl)oxy)methyl)-5-nitrobenzyl)(prop-2-yn-1-yl)carbamate

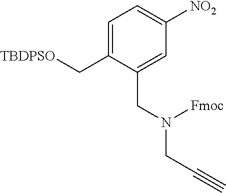

A solution of N-(2-(((tert-butyldiphenylsilyl)oxy)methyl)-5-nitrobenzyl)prop-2-yn-1-amine (9 g, 19.62 mmol, 1 eq) and Fmoc-OSU (7.28 g, 21.59 mmol, 1.1 eq) in dioxane (90 mL) was added sat. NaHCO₃ (90 mL) and the white suspension was stirred at 20° C. for 12 hr. The reaction mixture was diluted with H₂O 150 mL and extracted twice with EtOAc (150 mL each time). The combined organic layers were washed with brine 200 mL, dried over anhydrous Na₂SO₄, filtered and concentrated under reduced pressure to give a residue. The residue was purified by flash silica gel chromatography (Eluent of 0-30% Ethyl acetate/Petroleum ether). (9H-fluoren-9-yl)methyl (2-(((tert-butyldiphenylsilyl)oxy)methyl)-5-nitrobenzyl)(prop-2-yn-1-yl)carbamate (7.7 g, 11.08 mmol, 56.48% yield, 98% purity) was obtained as a white solid.

1H NMR (400 MHz, CHLOROFORM-d) δ ppm 1.12 (s, 9H) 2.17 (br d, J=14.31 Hz, 1H) 3.87-4.97 (m, 9H) 6.98-8.28 (m, 21H).

### Synthesis of (9H-fluoren-9-yl)methyl (5-amino-2-(((tert-butyldiphenylsilyl)oxy)methyl)benzyl)(prop-2-yn-1-yl)carbamate

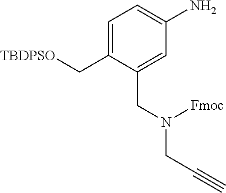

To an ice bath cooled solution of (9H-fluoren-9-yl)methyl (2-(((tert-butyldiphenylsilyl)oxy)methyl)-5-nitrobenzyl)(prop-2-yn-1-yl)carbamate (5.0 g, 7.34 mmol, 1.0 equiv.) in 10% AcOH/CH₂Cl₂ (100 mL) was added Zn (7.20 g, 110 mmol, 15 equiv.). The ice bath was removed and the resulting mixture stirred for 2 hours at which time it was filtered through a pad of celite. The volatiles were removed in vacuo and the residue was dissolved in EtOAc, was washed with NaHCO₃(sat.), NaCl(sat.), dried over MgSO₄, filtered, concentrated and after ISCO SiO₂ chromatography (0-75% EtOAc/Heptanes) (9H-fluoren-9-yl)methyl (5-amino-2-(((tert-butyldiphenylsilyl)oxy)methyl)benzyl)(prop-2-yn-1-yl)carbamate was obtained (2.99 g, 62%). LCMS: MH+=651.6; Rt=3.77 min (5 min acidic method-Method C).

### Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

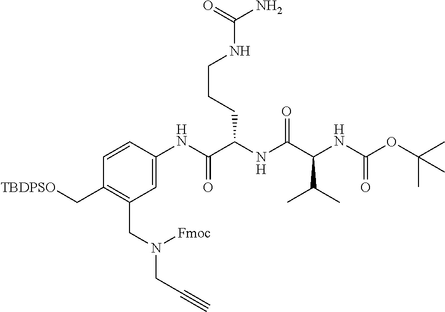

To (9H-fluoren-9-yl)methyl (5-amino-2-(((tert-butyldiphenylsilyl)oxy)methyl)benzyl)(prop-2-yn-1-yl)carbamate (2.99 g, 4.59 mmol, 1.0 equiv) and (S)-2-((S)-2-((tert-butoxycarbonyl)amino)-3-methylbutanamido)-5-ureidopentanoic acid (1.72 g, 4.59 mmol, 1.0 equiv.) in CH₂Cl₂ (40 mL) was added ethyl 2-ethoxyquinoline-1(2H)-carboxylate (2.27 g, 9.18 mmol, 2.0 equiv.). After stirring for 10 minutes, MeOH (1 mL) was added and the solution became homogeneous. The reaction was stirred for 16 hours, the volatiles were removed in vacuo and after purification by ISCO SiO₂ chromatography (0-15% MeOH/CH₂Cl₂) (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained (2.78 g, 60%). LCMS: MH+=1008.8; Rt=3.77 min (5 min acidic method-Method C).

### Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

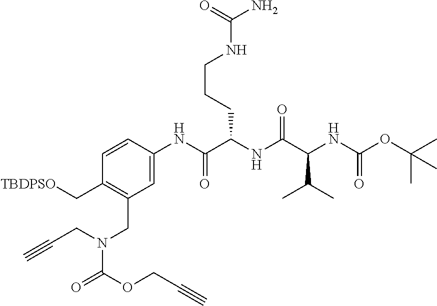

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (1.60 g, 1.588 mmol, 1.0 equiv.) was added 2M dimethylamine in MeOH (30 mL, 60 mmol, 37 equiv.) and THF (10 mL). After standing for 3 hours the volatiles were removed in vacuo and the residue was triturated with Et₂O to remove Fmoc deprotection byproducts. To the resulting solid was added CH₂Cl₂ (16 mL) and pyridine (4 mL) and to the heterogeneous solution was added propargyl chloroformate (155 μL, 1.588 mmol, 1.0 equiv.). After stirring for 30 minutes additional propargyl chloroformate (155 μL, 1.588 mmol, 1.0 equiv.) was added. After stirring for an additional 20 minutes MeOH (1 mL) was added to quench the remaining chloroformate and the volatiles were removed in vacuo. Upon purification by ISCO SiO₂ chromatography (0-15% MeOH/CH₂Cl₂) prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained (984 mg, 71%). LCMS: MH+=867.8; Rt=3.40 min (5 min acidic method-Method C).

### Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate

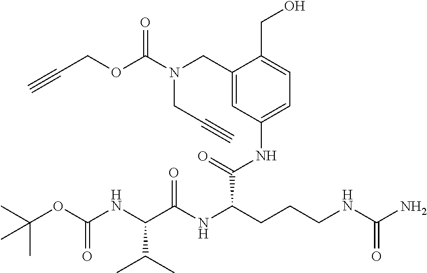 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1CO]

To a solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (984 mg, 1.135 mmol, 1.0 equiv.) in THF (7.5 mL) was added 1.0 M tetrabutylammonium fluoride in THF (2.27 mL, 2.27 mmol, 2.0 equiv.). After standing for 6 hours the volatiles were removed in vacuo, the residue was purified by ISCO SiO₂ chromatography (0-40% MeOH/CH₂Cl₂) and prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate was obtained (629 mg, 88%). LCMS: MH+=629.6; Rt=1.74 min (5 min acidic method-Method C).

### Synthesis of 2-(hydroxymethyl)-N-methyl-5-nitrobenzamide

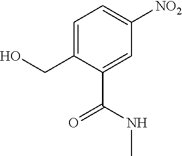

To a stirred suspension of 6-nitroisobenzofuran-1(3H)-one (500 g, 2.79 mol) in MeOH (1500 mL) was added MeNH₂ (3.00 kg, 29.94 mol, 600 mL, 31.0% purity) at 25° C. and stirred for 1 h. The solid was filtered and washed with water twice (600 mL) and dried under high vacuum to get a residue. The product 2-(hydroxymethyl)-N-methyl-5-nitrobenzamide (560 g, crude) was obtained as white solid. LCMS: RT=0.537 min, MS m/z=193.2. 1H NMR: 400 MHz DMSO δ 8.57 (br d, J=4.4 Hz, 1H), 8.31 (dd, J=2.4, 8.6 Hz, 1H), 8.21 (d, J=2.4 Hz, 1H), 7.86 (d, J=8.8 Hz, 1H), 5.54 (t, J=5.6 Hz, 1H), 4.72 (d, J=5.5 Hz, 2H), 2.78 (d, J=4.4 Hz, 3H).

### Synthesis of (2-((methylamino)methyl)-4-nitrophenyl)methanol

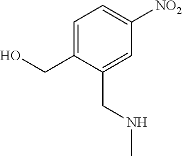

A solution of 2-(hydroxymethyl)-N-methyl-5-nitrobenzamide (560 g, 2.66 mol) in THF (5000 mL) was cooled to 0° C., then BH₃-Me₂S (506 g, 6.66 mol) (2.0 M in THF) was added drop wise for 60 min and the mixture was heated to 70° C. for 5 h. LCMS showed the starting material was consumed. After completion, 4M HCl (1200 mL) in Methanol was added to the reaction mixture at 0° C. and heated at 65° C. for 8 h. The reaction mixture was cooled to 0° C., the solid was filtered and concentrated in reduce pressure. (2-((methylamino)methyl)-4-nitrophenyl)methanol was obtained as a white solid (520 g). LCMS: RT=0.742 min, MS m/z=197.1 [M+H]+. ¹H NMR: 400 MHz DMSO δ 9.25 (br s, 2H), 8.37 (d, J=2.4 Hz, 1H), 8.14 (dd, J=2.4, 8.5 Hz, 1H), 7.63 (d, J=8.4 Hz, 1H), 5.72 (br s, 1H), 4.65 (s, 2H), 4.15 (br s, 2H), 2.55-2.45 (m, 3H)

### Synthesis of 1-(2-(((tert-butyldiphenylsilyl)oxy)methyl)-5-nitrophenyl)-N-methylmethanamine

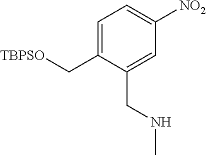

A solution of (2-((methylamino)methyl)-4-nitrophenyl)methanol (520 g, 2.65 mol) and imidazole (721 g, 10.6 mol) in DCM (2600 mL) was cooled to 0° C. then TBDPS-CI (1.09 kg, 3.98 mol, 1.02 L) was added drop wise and the mixture was stirred for 2 h. The mixture was poured into ice cold water (1000 mL) and extracted with ethyl acetate. The combined organic layers were washed with brine, dried over Na₂SO₄, filtered and evaporated under vacuum to give a crude product. The crude product was purified by chromatography on a silica gel eluted with ethyl acetate:Petroleum ether (from 10/1 to 1) to give a residue. 1-(2-(((tert-butyldiphenylsilyl)oxy)methyl)-5-nitrophenyl)-N-methylmethanamine (600 g) was obtained as a yellow liquid. LCMS: product: RT=0.910 min, MS m/z=435.2 [M+H]+ 1H NMR: 400 MHz CDCl3 δ 8.23 (d, J=2.4 Hz, 1H), 8.15 (dd, J=2.4, 8.4 Hz, 1H), 7.76 (d, J=8.4 Hz, 1H), 7.71-7.66 (m, 4H), 7.50-7.37 (m, 6H), 4.88 (s, 2H), 3.65 (s, 2H), 2.39 (s, 3H), 1.12 (s, 9H)

### Synthesis of (9H-fluoren-9-yl)methyl (2-(((tert-butyldiphenylsilyl)oxy)methyl)-5-nitrobenzyl)(methyl)carbamate

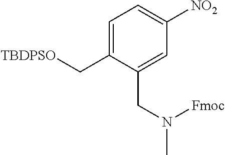

To a solution of 1-(2-(((tert-butyldiphenylsilyl)oxy)methyl)-5-nitrophenyl)-N-methylmethanamine (400 g, 920.3 mmol) in THF (4000 mL) was added Fmoc-OSU (341.5 g, 1.01 mol) and Et₃N (186.2 g, 1.84 mol, 256.2 mL), and the mixture was stirred at 25° C. for 1 h. The mixture was poured into water (1600 mL) and extracted twice with ethyl acetate (1000 mL). The combined organic layers were washed with brine, dried over Na₂SO₄, filtered and evaporated under vacuum to give crude product. The crude product was purified by chromatography on a silica gel eluted with petroleum ether:ethyl acetate (from 1/0 to 1/1) to give (9H-fluoren-9-yl)methyl (2-(((tert-butyldiphenylsilyl)oxy)methyl)-5-nitrobenzyl)(methyl)carbamate (405 g) as a white solid. LCMS: RT=0.931 min, MS m/z=657.2 [M+H]+. 1H NMR: 400 MHz CDCl3 δ 8.21-7.96 (m, 1H), 7.87-7.68 (m, 3H), 7.68-7.62 (m, 4H), 7.62-7.47 (m, 2H), 7.47-7.28 (m, 9H), 7.26-7.05 (m, 2H), 4.81 (br s, 1H), 4.62-4.37 (m, 4H), 4.31-4.19 (m, 1H), 4.08-3.95 (m, 1H), 2.87 (br d, J=5.2 Hz, 3H), 1.12 (s, 9H).

### Synthesis of (9H-fluoren-9-yl)methyl (5-amino-2-(((tert-butyldiphenylsilyl)oxy)methyl)benzyl)(methyl)carbamate

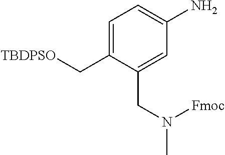

A solution of (9H-fluoren-9-yl)methyl (2-(((tert-butyldiphenylsilyl)oxy)methyl)-5-nitrobenzyl)(methyl)carbamate (3.0 g, 4.57 mmol, 1.0 equiv.) in MeOH (90 mL) and EtOAc (30 mL) was degassed and purged to a balloon of N₂ via three way stopcock. After repeating degas/N₂ purge 2×, 10% Pd/C deGussa type (0.486 g, 0.457 mmol, 0.1 equiv.) was added. The resulting mixture was degassed and purged to a balloon of 2H₂ via three way stopcock. After repeating degas/H₂ purge 2×, the reaction stirred under the balloon pressure of H₂ for 4 hours. The reaction was degassed and purged to N₂, filtered through a pad of celite eluting further with MeOH. After removal of the volatiles in vacuo and pumping on high vac (9H-fluoren-9-yl)methyl (5-amino-2-(((tert-butyldiphenylsilyl)oxy)methyl)benzyl)(methyl)carbamate was obtained (2.78 g, 97%). LCMS: MH+=627.7; Rt=1.59 min (2 min acidic method-Method A). ¹H NMR: 400 MHz CDCl3 δ 7.80 (br d, J=7.2 Hz, 1H), 7.74-7.67 (m, 5H), 7.64 (br d, J=6.8 Hz, 1H), 7.49-7.30 (m, 10H), 7.23-7.06 (m, 2H), 6.61-6.41 (m, 2H), 4.66 (br d, J=7.2 Hz, 2H), 4.55 (s, 2H), 4.51-4.34 (m, 2H), 4.32-4.10 (m, 1H), 3.66 (br s, 2H), 2.96-2.78 (m, 3H), 1.07 (s, 9H).

### Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

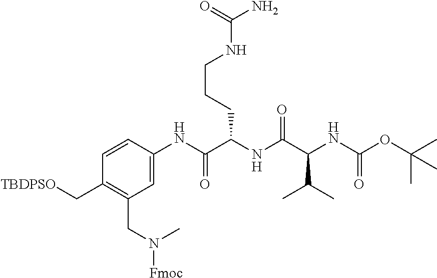

To (9H-fluoren-9-yl)methyl (5-amino-2-(((tert-butyldiphenylsilyl)oxy)methyl)benzyl)(methyl)carbamate (2.86 g, 4.56 mmol, 1.0 equiv) and (S)-2-((S)-2-((tert-butoxycarbonyl)amino)-3-methylbutanamido)-5-ureidopentanoic acid (1.71 g, 4.56 mmol, 1.0 equiv.) in 2:1 CH2Cl2/MeOH (60 mL) was added ethyl 2-ethoxyquinoline-1(2H)-carboxylate (2.256 g, 9.12 mmol, 2.0 equiv.). The homogeneous solution was stirred for 16 hours at which time additional (S)-2-((S)-2-((tert-butoxycarbonyl)amino)-3-methylbutanamido)-5-ureidopentanoic acid (0.340 g, 0.2 equiv.) and ethyl 2-ethoxyquinoline-1(2H)-carboxylate (0.452 g, 0.4 equiv.) were added to drive the reaction to completion. After stirring for an additional 5 hours the volatiles were removed in vacuo and after purification by ISCO SiO₂ chromatography (0-5% MeOH/CH₂Cl₂) (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate was obtained (2.95 g, 65%). LCMS: MH+=984.1; Rt=1.54 min (2 min acidic method-Method A).

### Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

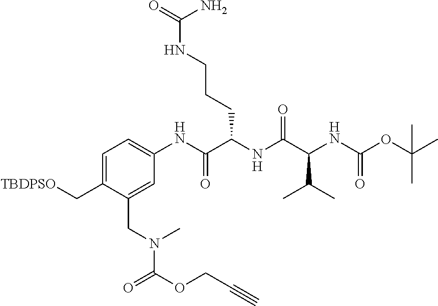

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (2.05 g, 2.085 mmol, 1.0 equiv) in THF (10 mL) was added 2.0 M dimethyl amine in MeOH (10.42 mL, 20.85 mmol, 10 equiv.). After stirring for 16 hours the volatiles were removed in vacuo. The residue was dissolved in CH₂Cl₂ (20 mL) and DIEA (0.533 mL, 4.17 mmol, 2 equiv.) and propargyl chloroformate (0.264 mL, 2.71 mmol, 1.3 equiv.) were added. After stirring at rt for 16 hours the reaction was diluted with CH₂Cl₂ (20 mL), was washed with NaHCO₃ (sat.), NaCl(sat.), dried over MgSO₄, filtered, concentrated and purified by ISCO SiO₂ chromatography (0-15% MeOH/CH₂Cl₂) to yield prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (1.04 grams, 59%). LCMS: MH+=843.8; Rt=1.35 min (2 min acidic method-Method A).

### Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate

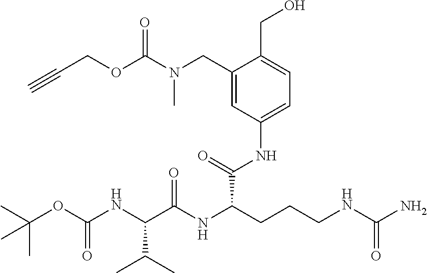 [SMILES: C#CCOC(=O)N(C)Cc1cc(NC(=O)[C@H](CCCNC(N)=O)NC(=O)[C@@H](NC(=O)OC(C)(C)C)C(C)C)ccc1CO]

To a 0° C. solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (1.6 g, 1.90 mmol, 1.0 equiv.) in THF (10.0 mL) was added 1.0 M tetrabutylammonium fluoride in THF (3.80 mL, 3.80 mmol, 2.0 equiv.). After warming to rt and stirring for 16 hours the volatiles were removed in vacuo, the residue was dissolved in EtOAc, was washed with NaHCO₃(sat.), with NaCl(sat.), dried over MgSO₄, filtered, concentrated and the residue was purified by ISCO SiO₂ chromatography (0-30% MeOH/CH₂Cl₂) to yield prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (1.0 g, 87%). LCMS: MH+=605.7; Rt=0.81 min (2 min acidic method-Method A).

### Synthesis of (9H-fluoren-9-yl)methyl (S)-(1-((4-(hydroxymethyl)-3-nitrophenyl)amino)-1-oxo-5-ureidopentan-2-yl)carbamate

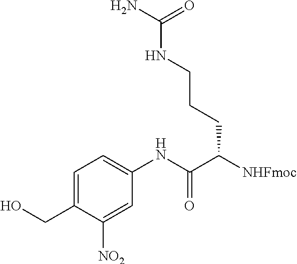

To a solution of (4-amino-2-nitrophenyl)methanol (10 g, 59.5 mmol, 1.0 equiv.), (9H-fluoren-9-yl)methyl (S)-(1-amino-1-oxo-5-ureidopentan-2-yl)carbamate (23.64 g, 59.5 mmol, 1.0 equiv.), and 1-hydroxy-7-azabenzotriazole (8.50 g, 62.4 mmol, 1.05 equiv.) in DMF (50 mL) was added 1-(3-dimethylaminopropyl)-3-ethylcarbodiimide (11.97 g, 62.4 mmol, 1.05 equiv.). After stirring at ambient temperature for 16 hours, the mixture was poured into water (4 L) and stirred for 30 minutes. The resulting solid was filtered, rinsed with water, and dried under vacuum. (9H-Fluoren-9-yl)methyl (S)-(1-((4-(hydroxymethyl)-3-nitrophenyl)amino)-1-oxo-5-ureidopentan-2-yl)carbamate was obtained (31.49 g, 57.5 mmol, 97%). LCMS: MH+=548; Rt=2.02 min (5 min acidic method-Method C).

### Synthesis of (S)-2-amino-N-(4-(hydroxymethyl)-3-nitrophenyl)-5-ureidopentanamide

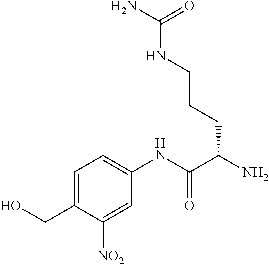 [SMILES: NC(=O)NCCC[C@H](N)C(=O)Nc1ccc(CO)c([N+](=O)[O-])c1]

To a solution of (9H-Fluoren-9-yl)methyl (S)-(1-((4-(hydroxymethyl)-3-nitrophenyl)amino)-1-oxo-5-ureidopentan-2-yl)carbamate (31.49 g, 57.5 mmol, 1.0 equiv.) in DMF (50 mL) was added dimethylamine (2 M in MeOH, 331 mL, 661 mmol, 11.5 equiv.). After stirring at ambient temperature for 24 hours, the volatiles were removed under vacuum and the resulting residue was triturated with diethyl ether (3×2 L). The resulting residue was dried under vacuum and (S)-2-amino-N-(4-(hydroxymethyl)-3-nitrophenyl)-5-ureidopentanamide was obtained (21.85 g, 57.5 mmol, 99%). LCMS: MH+=326.4; Rt=0.35 min (2 min acidic method-Method A).

### Synthesis of tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate

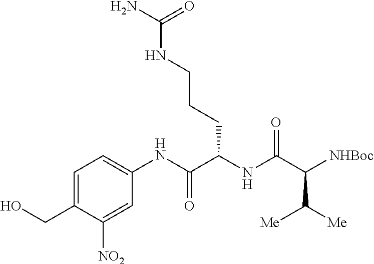 [SMILES: CC(C)[C@H](NC(=O)OC(C)(C)C)C(=O)N[C@@H](CCCNC(N)=O)C(=O)Nc1ccc(CO)c([N+](=O)[O-])c1]

To a solution of (S)-2-amino-N-(4-(hydroxymethyl)-3-nitrophenyl)-5-ureidopentanamide (10.89 g, 28.8 mmol, 1.0 equiv.), (tert-butoxycarbonyl)-L-valine (6.25 g, 28.8 mmol, 1.0 equiv.), and 1-hydroxy-7-azabenzotriazole (3.92 g, 28.8 mmol, 1.0 equiv.) in DMF (40 mL) was added 1-(3-dimethylaminopropyl)-3-ethylcarbodiimide (5.52 g, 28.8 mmol, 1.0 equiv.). After stirring at ambient temperature for 24 hours, the mixture was added dropwise to water (2 L), stirred for 30 minutes, and cooled to 4° C. overnight. The mixture was saturated with NaCl, and the resulting solids were filtered off and dried under vacuum. Tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate was obtained (11.96 g, 22.8 mmol, 79%). LCMS: MH+=525.4; Rt=0.79 min (2 min acidic method-Method A).

### Synthesis of tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate

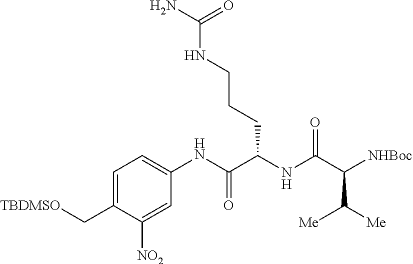 [SMILES: CC(C)[C@H](NC(=O)OC(C)(C)C)C(=O)N[C@@H](CCCNC(N)=O)C(=O)Nc1ccc(CO[Si](C)(C)C(C)(C)C)c([N+](=O)[O-])c1]

To a suspension of tert-butyl ((S)-1-(((S)-1-((4-(hydroxymethyl)-3-nitrophenyl)amino)-1-oxo-5-ureidopentan-2-yl)amino)-3-methyl-1-oxobutan-2-yl)carbamate (11.96 g, 22.8 mmol, 1.0 equiv.) and imidazole (15.52 g, 228 mmol, 10 equiv.) in DMF (31 mL) was added tert-butyldimethylchlorosilane (13.68 g, 90.76 mmol, 4.0 equiv.). The resulting mixture was stirred at ambient temperature for 48 hours, then heated at 45° C. for 4 hours. The mixture was poured into water and stirred for 96 hours. Solids were filtered and washed with water (2×100 mL) and dried under vacuum. After purification by SiO₂ ISCO chromatography (0-30% methanol/dichloromethane), tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate was obtained (8.02 g, 12.56 mmol, 55%). LCMS: MH+=639.6; Rt=1.22 min (2 min acidic method-Method A).

### Synthesis of tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate

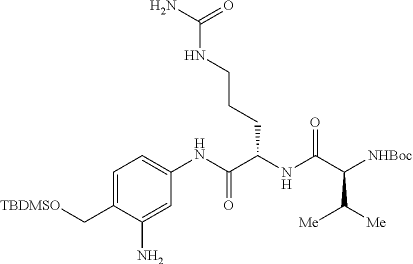 [SMILES: CC(C)[C@H](NC(=O)OC(C)(C)C)C(=O)N[C@@H](CCCNC(N)=O)C(=O)Nc1ccc(CO[Si](C)(C)C(C)(C)C)c(N)c1]

To a solution of tert-butyl ((S)-1-(((S)-1-((4-(((tert-butyldimethylsilyl)oxy)methyl)-3-nitrophenyl)amino)-1-oxo-5-ureidopentan-2-yl)amino)-3-methyl-1-oxobutan-2-yl)carbamate (8.02 g, 12.56 mmol, 1.0 equiv.) in methanol (250 mL) under a nitrogen atmosphere was added palladium on carbon (10 wt %, 2.00 g, 1.884 mmol, 0.15 equiv.). The mixture was placed under 1 atm dihydrogen and allowed to stir at ambient temperature for 18 hours. The mixture was filtered through celite and dried under vacuum. After purification by SiO₂ ISCO chromatography (0-40% methanol/dichloromethane), tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate was obtained (4.82 g, 7.92 mmol, 63%). LCMS: MH+=609.6; Rt=2.65 min (5 min acidic method-Method C).

### Synthesis of tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate

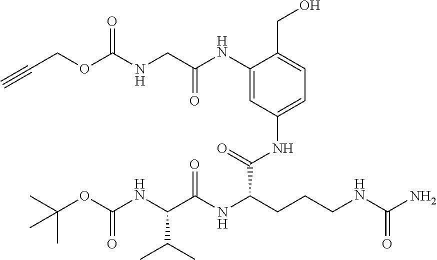 [SMILES: C#CCOC(=O)NCC(=O)Nc1cc(NC(=O)[C@H](CCCNC(N)=O)NC(=O)[C@@H](NC(=O)OC(C)(C)C)C(C)C)ccc1CO]

To a solution of glycine (3.19 g, 42.5 mmol, 1.0 equiv.) in 2 M aqueous sodium hydroxide solution (63.3 mL, 127 mmol NaOH, 3.0 equiv.) was added propargyl chloroformate (5.0 g, 42.5 mmol, 1.0 equiv.). The resulting mixture was stirred at ambient temperature for 3 hours. The mixture was extracted with ethyl acetate (3×250 mL). The combined organic layers were dried over magnesium sulfate, filtered and the volatiles removed under vacuum. After drying, ((prop-2-yn-1-yloxy)carbonyl)glycine,

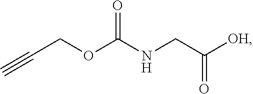

was obtained (3.97 g, 25.3 mmol, 59%). ¹H NMR (400 MHz, DMSO-d₆) δ ppm 3.48 (t, J=2.40 Hz, 1H) 3.66 (d, J=6.19 Hz, 2H) 4.63 (d, J=2.40 Hz, 2H) 7.63 (t, J=6.13 Hz, 1H) 12.57 (br s, 1H). To a solution of tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate (2.7 g, 4.43 mmol, 1.0 equiv.) in DMF (5 mL) were added ((prop-2-yn-1-yloxy)carbonyl)glycine (0.732 g, 4.66 mmol, 1.05 equiv.), 1-hydroxy-7-azabenzotriazole (0.664 g, 4.88 mmol, 1.1 equiv.), and 1-(3-dimethylaminopropyl)-3-ethylcarbodiimide hydrochloride (0.935 g, 4.88 mmol, 1.1 equiv). The resulting mixture was stirred at ambient temperature for 1 hour, then dripped into water (500 mL) and stirred for a further 20 minutes. The resulting precipitate was filtered, washed with water, and dried under vacuum. After purification by SiO₂ ISCO chromatography (0-50% methanol/dichloromethane), tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate (LI-4) was obtained (1.52 g, 2.40 mmol, 54%). LCMS: MH+=634.6; Rt=1.97 min (5 min acidic method-Method C). ¹H NMR (400 MHz, DMSO-d₆) δ ppm 0.76-0.91 (m, 6H) 1.30-1.47 (m, 11H) 1.51-1.73 (m, 2H) 1.87-2.00 (m, 1H) 2.89-3.07 (m, 2H) 3.50 (t, J=2.32 Hz, 1H) 3.73-3.87 (m, 3H) 4.37-4.47 (m, 3H) 4.65 (d, J=2.45 Hz, 2H) 5.30 (t, J=5.44 Hz, 1H) 5.38 (s, 2H) 5.96 (t, J=5.81 Hz, 1H) 6.72 (br d, J=8.93 Hz, 1H) 7.25 (d, J=8.44 Hz, 1H) 7.45 (dd, J=8.25, 2.02 Hz, 1H) 7.78 (br t, J=5.87 Hz, 1H) 7.87-8.00 (m, 2H) 9.51 (s, 1H) 10.04 (s, 1H). To a solution of tert-butyl ((S)-1-(((S)-1-((3-amino-4-(((tert-butyldimethylsilyl)oxy)methyl)phenyl)amino)-1-oxo-5-ureidopentan-2-yl)amino)-3-methyl-1-oxobutan-2-yl)carbamate (2.7 g, 4.43 mmol, 1.0 equiv.) in DMF (5 mL) were added ((prop-2-yn-1-yloxy)carbonyl)glycine (0.732 g, 4.66 mmol, 1.05 equiv.), 1-hydroxy-7-azabenzotriazole (0.664 g, 4.88 mmol, 1.1 equiv.), and 1-(3-dimethylaminopropyl)-3-ethylcarbodiimide hydrochloride (0.935 g, 4.88 mmol, 1.1 equiv). The resulting mixture was stirred at ambient temperature for 1 hour, then dripped into water (500 mL) and stirred for a further 20 minutes. The resulting precipitate was filtered, washed with water, and dried under vacuum. After purification by SiO₂ ISCO chromatography (0-50% methanol/dichloromethane), tert-butyl ((S)-1-(((S)-1-((4-(hydroxymethyl)-3-(2-(((prop-2-yn-1-yloxy)carbonyl)amino)acetamido)phenyl)amino)-1-oxo-5-ureidopentan-2-yl)amino)-3-methyl-1-oxobutan-2-yl)carbamate was obtained (1.52 g, 2.40 mmol, 54%). LCMS: MH+=634.6; Rt=1.97 min (5 min acidic method-Method C). ¹H NMR (400 MHz, DMSO-d₆) δ ppm 0.76-0.91 (m, 6H) 1.30-1.47 (m, 11H) 1.51-1.73 (m, 2H) 1.87-2.00 (m, 1H) 2.89-3.07 (m, 2H) 3.50 (t, J=2.32 Hz, 1H) 3.73-3.87 (m, 3H) 4.37-4.47 (m, 3H) 4.65 (d, J=2.45 Hz, 2H) 5.30 (t, J=5.44 Hz, 1H) 5.38 (s, 2H) 5.96 (t, J=5.81 Hz, 1H) 6.72 (br d, J=8.93 Hz, 1H) 7.25 (d, J=8.44 Hz, 1H) 7.45 (dd, J=8.25, 2.02 Hz, 1H) 7.78 (br t, J=5.87 Hz, 1H) 7.87-8.00 (m, 2H) 9.51 (s, 1H) 10.04 (s, 1H).

### Synthesis of 2-(((tert-butyldiphenylsilyl)oxy)methyl)-5-nitro-N,N-di(prop-2-yn-1-yl)benzamide

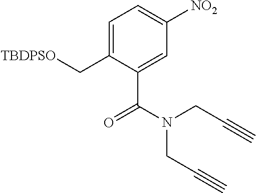

To a solution of 2-(((tert-butyldiphenylsilyl)oxy)methyl)-5-nitrobenzoic acid (1.00 g, 2.30 mmol, 1.0 equiv.) and dipropargylamine (0.257 g, 2.76 mmol, 1.2 equiv.) in dichloromethane (6 mL) were added (1-[bis(dimethylamino)methylene]-1H-1,2,3-triazolo[4,5-b]pyridinium 3-oxid hexafluorophosphate (1.048 g, 2.76 mmol, 1.2 equiv.) and N,N-diisopropylethylamine (0.445 g, 3.44 mmol, 1.5 equiv.). The resulting mixture was stirred at ambient temperature for 1 hour, then diluted with water, extracted with diethyl ether (3×25 mL), dried over sodium sulfate and concentrated. After purification by SiO₂ ISCO chromatography (0-100% ethyl acetate/heptanes), 2-(((tert-butyldiphenylsilyl)oxy)methyl)-5-nitro-N,N-di(prop-2-yn-1-yl)benzamide was obtained (1.08 g, 2.115 mmol, 92%). 1H NMR (400 MHz, Chloroform-d) δ 8.35 (dd, J=8.6, 2.3 Hz, 1H), 8.20 (d, J=2.3 Hz, 1H), 8.02-7.92 (m, 1H), 7.71-7.62 (m, 4H), 7.51-7.35 (m, 6H), 4.87 (s, 2H), 4.39 (s, 2H), 3.80 (s, 2H), 2.21 (s, 1H), 2.08 (d, J=7.7 Hz, 1H), 1.13 (s, 9H).

### Synthesis of 5-amino-2-(((tert-butyldiphenylsilyl)oxy)methyl)-N,N-di(prop-2-yn-1-yl)benzamide

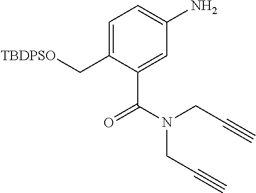

To a stirred suspension of 2-(((tert-butyldiphenylsilyl)oxy)methyl)-5-nitro-N,N-di(prop-2-yn-1-yl)benzamide (1.08 g, 2.115 mmol, 1.0 equiv.) in ethanol (4 mL) and water (4 mL) was added zinc powder (0.553 g, 8.46 mmol, 4 equiv.) and ammonium chloride (0.453 g, 8.46 mmol, 4 equiv.). The resulting mixture was stirred at ambient temperature for 24 hours, then diluted with water and extracted with ethyl acetate (3×25 mL). The combined organic layers were dried over sodium sulfate, filtered and concentrated. After drying under vacuum, 5-amino-2-(((tert-butyldiphenylsilyl)oxy)methyl)-N,N-di(prop-2-yn-1-yl)benzamide was obtained (972 mg, 2.02 mmol, 96%). LCMS: MH+=481.4; Rt=1.33 min (2 min acidic method-Method A).

### Synthesis of (S)-5-(2-amino-5-ureidopentanamido)-2-(((tert-butyldiphenylsilyl)oxy)methyl)-N,N-di(prop-2-yn-1-yl)benzamide

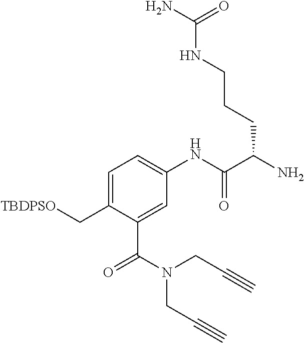

To a solution of 5-amino-2-(((tert-butyldiphenylsilyl)oxy)methyl)-N,N-di(prop-2-yn-1-yl)benzamide (972 mg, 2.02 mmol, 1.0 equiv.), (9H-fluoren-9-yl)methyl (S)-(1-amino-1-oxo-5-ureidopentan-2-yl)carbamate (804 mg, 2.02 mmol, 1.0 equiv.), and (1-[bis(dimethylamino)methylene]-1H-1,2,3-triazolo[4,5-b]pyridinium 3-oxid hexafluorophosphate (846 mg, 2.22 mmol, 1.1 equiv.) in DMF (4 mL) was added N,N-diisopropylethylamine (0.53 mL, 3.03 mmol, 1.5 equiv.). The resulting mixture was stirred at ambient temperature for 18 hours, then poured into water (400 mL) and stirred for 3 hours. The precipitate was filtered and dried under vacuum, then dissolved in a 2 M solution of dimethylamine in tetrahydrofuran (2.02 mL, 4.04 mmol, 2 equiv.) and stirred at ambient temperature for 4 hours. The volatiles were removed under vacuum and after purification by SiO2 ISCO chromatography, (S)-5-(2-amino-5-ureidopentanamido)-2-(((tert-butyldiphenylsilyl)oxy)methyl)-N,N-di(prop-2-yn-1-yl)benzamide was obtained (1.018 g, 1.596 mmol, 79%). LCMS: MH+=638.6; Rt=1.22 min (2 min acidic method-Method A).

### Synthesis of tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate

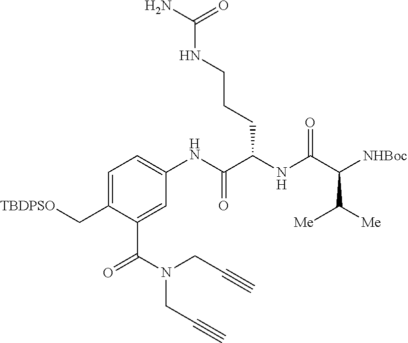

To a solution of (S)-5-(2-amino-5-ureidopentanamido)-2-(((tert-butyldiphenylsilyl)oxy)methyl)-N,N-di(prop-2-yn-1-yl)benzamide (1.00 g, 1.568 mmol, 1.0 equiv.), (tert-butoxycarbonyl)-L-valine (0.341 g, 1.568 mmol, 1.0 equiv.), and (1-[bis(dimethylamino)methylene]-1H-1,2,3-triazolo[4,5-b]pyridinium 3-oxid hexafluorophosphate (0.656 g, 1.725 mmol, 1.1 equiv.) in DMF (3 mL) was added N,N-diisopropylethylamine (0.41 mL, 2.352 mmol, 1.5 equiv). After stirring at ambient temperature for 1 hour, the mixture was diluted with water (30 mL) and brine (30 mL) and extracted with ethyl acetate (3×50 mL). The combined organic layers were dried over sodium sulfate, filtered and concentrated. After purification of the resulting residue by SiO₂ ISCO chromatography (0-50% methanol/dichloromethane), tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate was obtained (1.30 g, 1.553 mmol, 99%). LCMS: MH+=837.5; Rt=1.32 min (2 min acidic method-Method A).

### Synthesis of tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate

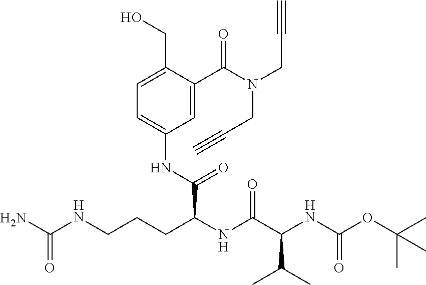 [SMILES: C#CCN(CC#C)C(=O)c1cc(NC(=O)[C@H](CCCNC(N)=O)NC(=O)[C@@H](NC(=O)OC(C)(C)C)C(C)C)ccc1CO]

To a stirred solution of tert-butyl ((S)-1-(((S)-1-((4-(((tert-butyldiphenylsilyl)oxy)methyl)-3-(di(prop-2-yn-1-yl)carbamoyl)phenyl)amino)-1-oxo-5-ureidopentan-2-yl)amino)-3-methyl-1-oxobutan-2-yl)carbamate (1.30 g, 1.553 mmol, 1.0 equiv.) in tetrahydrofuran (5 mL), a 1 M solution of tetrabutylammonium fluoride in tetrahydrofuran (3.11 mL, 3.11 mmol, 2.0 equiv.) was added dropwise. After stirring at ambient temperature for 18 hours, the solvent was removed under vacuum. After purification by SiO₂ ISCO chromatography (0-50% methanol/dichloromethane), tert-butyl ((S)-1-(((S)-1-((3-(di(prop-2-yn-1-yl)carbamoyl)-4-(hydroxymethyl)phenyl)amino)-1-oxo-5-ureidopentan-2-yl)amino)-3-methyl-1-oxobutan-2-yl)carbamate was obtained (0.703 g, 1.174 mmol, 76%). LCMS: MH+=599.4; Rt=0.76 min (2 min acidic method-Method A). 1H NMR (400 MHz, Methanol-d4) δ 7.71-7.59 (m, 2H), 7.52-7.43 (m, 1H), 4.51 (d, J=29.4 Hz, 4H), 4.11-4.04 (m, 2H), 3.95-3.85 (m, 1H), 3.28-3.06 (m, 2H), 2.76 (m, 2H), 2.11-2.03 (m, 1H), 1.97-1.83 (m, 1H), 1.75 (dtd, J=14.2, 9.4, 5.1 Hz, 1H), 1.70-1.51 (m, 3H), 1.44 (m, 9H), 1.00-0.90 (m, 6H).

### Synthesis of (9H-fluoren-9-yl)methyl (S)-(5-(2-((tert-butoxycarbonyl)amino)-5-ureidopentanamido)-2-(((tert-butyldiphenylsilyl)oxy)methyl)benzyl)(methyl)carbamate

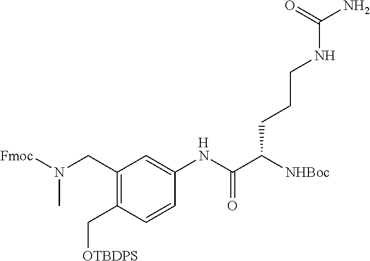

A solution of (S)-2-((tert-Butoxycarbonyl)amino)-5-ureidopentanoic acid (17.57 grams, 63.8 mmol), (9H-fluoren-9-yl)methyl (5-amino-2-(((tert-butyldiphenylsilyl)oxy)methyl)benzyl)(methyl)carbamate (40 grams, 63.8 mmol) and HOAT (1.0 M in DMA, 63.8 mL, 63.8 mmol) in DMF (36 mL) was stirred until homogeneous, cooled in a 0° C. bath and EDC (12.23 grams, 63.8 mmol) was added. The reaction was left stirring as the bath was allowed to warm to rt overnight. After 16 hours, the reaction solution was dripped into 4 L H₂O with stirring over ca. 30 minutes at which time the ppt was filtered, rinsed with H₂O (1 L) and air dried under vacuum. The wet cake was dissolved in 15% iPrOH/EtOAc (ca. 1.8 L) and washed with H₂O (250 mL), with NaHCO₃(sat.) (250 mL) and with NaCl(sat.) (250 mL), dried over MgSO₄, filtered, concentrated and pumped on to yield (9H-fluoren-9-yl)methyl (S)-(5-(2-((tert-butoxycarbonyl)amino)-5-ureidopentanamido)-2-(((tert-butyldiphenylsilyl)oxy)methyl)benzyl)(methyl)carbamate (54.47 grams, 96% yield). LC/MS: MH⁺=884.7, Rt=3.82 min (5 min. acidic method).

### Synthesis of (9H-fluoren-9-yl)methyl (S)-(5-(2-((tert-butoxycarbonyl)amino)-5-ureidopentanamido)-2-(hydroxymethyl)benzyl)(methyl)carbamate

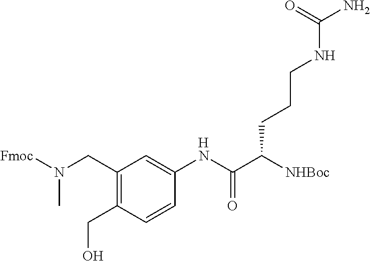

To a solution of (9H-fluoren-9-yl)methyl (S)-(5-(2-((tert-butoxycarbonyl)amino)-5-ureidopentanamido)-2-(((tert-butyldiphenylsilyl)oxy)methyl)benzyl)(methyl)carbamate (33.29 grams, 37.7 mmol) in THF (45 mL) was added AcOH (12.93 mL, 226 mmol) followed by 1.0 M TBAF in THF (56.6 mL, 56.6 mmol). After stirring for 72 hours, the volatiles were removed in vacuo and the residue was partitioned between 15% iPrOH/EtOAc (1.5 L) and H₂O (300 mL), mixed, separated, washed further with H₂O (5×350 mL), with NaHCO₃(sat.) (2×300 mL), with NaCl(sat.) (300 mL), dried over MgSO₄, filtered, concentrated, pumped on, triturated with Et2O and filtered to yield (9H-fluoren-9-yl)methyl (S)-(5-(2-((tert-butoxycarbonyl)amino)-5-ureidopentanamido)-2-(hydroxymethyl)benzyl)(methyl)carbamate (19.17 grams, 79% yield). LC/MS: MH⁺=646.7, Rf=2.28 min. (5 min acidic method).

### Synthesis of (9H-fluoren-9-yl)methyl (S)-(5-(2-amino-5-ureidopentanamido)-2-(hydroxymethyl)benzyl)(methyl)carbamate

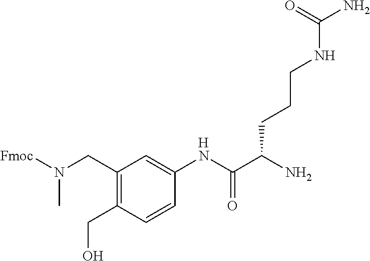

(9H-fluoren-9-yl)methyl (S)-(5-(2-((tert-butoxycarbonyl)amino)-5-ureidopentanamido)-2-(hydroxymethyl)benzyl)(methyl)carbamate (15 g, 23.23 mmol) was dissolved in DCM (30 mL), cooled in ice bath and TFA (28.5 mL, 372 mmol) was added slowly over 5 min. The reaction was kept stirring in the ice bath for 1 hr. The reaction was slowly warmed up to RT and stirred at RT for an 1 hour. The volatiles were removed in-vacuo. The product was retaken in dicholoroethane (210 mL). The resulting solution was removed in-vacuo. The process was repeated 2 more times (3 times in total) to obtain a dark yellow solid. The solid was suspended in diethyl ether (300 mL). It was sonicated for 10 min then decanted. The process was repeated 2 more times. The solid was collected by vacuum filtration. After air drying overnight under vacuum, the solid was dissolved in MeOH (100 mL) and this solution was added dropwise over ca. 20 minutes to 7N ammonia in MeOH (201 ml, 402 mmol). After stirring for 30 minutes, the volatiles were removed in vacuo and, after trituration with Et2O, (9H-fluoren-9-yl)methyl (S)-(5-(2-amino-5-ureidopentanamido)-2-(hydroxymethyl)benzyl)(methyl)carbamate (15.75 grams) was obtained. LC/MS: MH⁺=546.5, Rt=1.43 min. (5 min acidic method). The material was used as is for next step.

### Synthesis of (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate

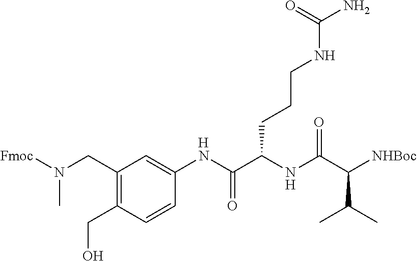

Boc-Val-OH (6.47 g, 29.8 mmol), EDC·HCl (5.48 g, 28.7 mmol) and HOAT (3.89 g, 28.7 mmol) were dissolved in DMF (35 mL) and stirred at RT for 10 min. at which time ((9H-fluoren-9-yl)methyl (S)-(5-(2-amino-5-ureidopentanamido)-2-(hydroxymethyl)benzyl)(methyl)carbamate (13 g, 23.83 mmol) was added. DIPEA (4.15 mL, 23.83 mmol) was added. After stirring for 30 minutes, the reaction mixture was added dropwise to 4 L of water under vigorous stirring. After 30 minutes, the precipitate was collected by vacuum filtration. The cake was washed with water (1200 mL). The product was air dried under vacuum, was triturated with MTBE, and dried under vacuum for to yield (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (12.8 g, 72% yield). LC/MS: MH⁺=745.7, 2.37 min. (5 min acidic method).

### Synthesis of (9H-fluoren-9-yl)methyl (5-((S)-2-((S)-2-(((allyloxy)carbonyl)amino)-3-methylbutanamido)-5-ureidopentanamido)-2-(hydroxymethyl)benzyl)(methyl)carbamate

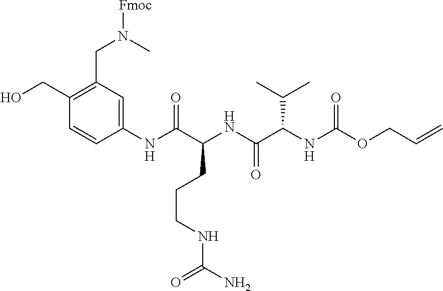

To a solution of (9H-fluoren-9-yl)methyl (S)-(5-(2-amino-5-ureidopentanamido)-2-(hydroxymethyl)benzyl)(methyl)carbamate (9.4 g, 14.25 mmol), HOAT (2.32 g, 17.1 mmol) and ((allyloxy)carbonyl)-L-valine (3.44 g, 17.10 mmol) in DMF (27 mL) was added DIEA (5.53 g, 42.7 mmol) and EDC (3.28 g, 17.1 mmol). After stirring for 2 hours, the solution was dripped into 3 L of H₂O with stirring. The solid formed was collected by vacuum filtration, rinsed further with H₂O and air dried under vacuum to yield (9H-fluoren-9-yl)methyl (5-((S)-2-((S)-2-(((allyloxy)carbonyl)amino)-3-methylbutanamido)-5-ureidopentanamido)-2-(hydroxymethyl)benzyl)(methyl)carbamate (7.69 g, 74% yield). LC/MS: MH⁺=729.6, Rt=2.27 min (5 min acidic method).

### Synthesis of (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate

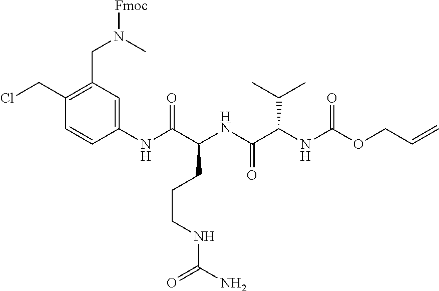

To a heterogeneous solution of (9H-fluoren-9-yl)methyl (5-((S)-2-((S)-2-(((allyloxy)carbonyl)amino)-3-methylbutanamido)-5-ureidopentanamido)-2-(hydroxymethyl)benzyl)(methyl)carbamate (4.0 g, 5.5 mmol) and NaHCO₃ (2.8 g, 33 mmol) in THF (55 mL) at rt was added thionyl chloride (1.0 mL, 14 mmol). After stirring for 1 hour, the solution was partitioned between EtOAc and NaHCO₃(sat.), extracted further with EtOAc, dried over MgSO₄, filtered, concentrated, purified by SiO₂ chromatography (0-20% iPrOH/CH₂Cl₂ eluant). After concentration, (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate (2.1 g, 51% yield) was obtained. LC/MS: MH⁺=747.6, Rt=2.72 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate

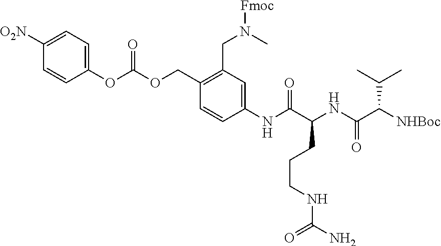

To a solution of (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (2.0 g, 2.685 mmol) and bis(4-nitrophenyl) carbonate (1.634 g, 5.37 mmol) in NMP (7 mL) was added DIEA (1.4 mL, 8.06 mmol). After stirring for 2 hours, the solution was diluted with DMSO and purified by RP-HPLC. After lyophilization, (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (2.0 g, 82% yield) was obtained. HRMS: MH⁺=910.4000, Rt=3.07 min (5 min acidic method).

### Synthesis of (9H-fluoren-9-yl)methyl (5-((S)-2-((S)-2-amino-3-methylbutanamido)-5-ureidopentanamido)-2-(hydroxymethyl)benzyl)(methyl)carbamate

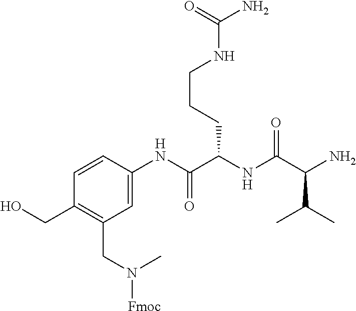

(9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (2.0 grams, 2.68 mmol) was treated with 25% TFA/CH₂Cl₂ (12 mL) in an ice bath. After stirring for one hour, the volatiles were removed in vacuo, dichloroethane was added and volatiles were removed in vacuo. The residue was dissolved in MeOH (20 mL) and the solution was added dropwise to 7M ammonia in MeOH (7.67 mL, 53.7 mmol). After stirring for 30 minutes, the volatiles were removed in vacuo, the residue was dissolved in MeOH and was purified by ISCO RP-HPLC (with no acidic modifier). Upon lyophilization, (9H-fluoren-9-yl)methyl (5-((S)-2-((S)-2-amino-3-methylbutanamido)-5-ureidopentanamido)-2-(hydroxymethyl)benzyl)(methyl)carbamate (1.162 g, 67% yield) was obtained. LC/MS: MH⁺=645.5, Rt=0.75 min (2 min acidic method).

### Synthesis of (9H-fluoren-9-yl)methyl (5-((S)-2-((S)-2-(2-azidoacetamido)-3-methylbutanamido)-5-ureidopentanamido)-2-(hydroxymethyl)benzyl)(methyl)carbamate

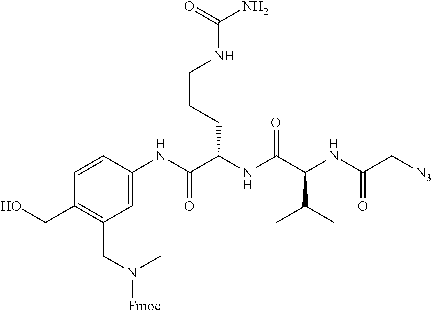

To a solution of (9H-fluoren-9-yl)methyl (5-((S)-2-((S)-2-amino-3-methylbutanamido)-5-ureidopentanamido)-2-(hydroxymethyl)benzyl)(methyl)carbamate (400 mg, 0.620 mmol), 2-azidoacetic acid (60.4 μl, 0.806 mmol) and HATU (307 mg, 0.806 mmol) in DMF (5 ml) was added DIPEA (433 μl, 2.481 mmol). After stirring for 30 min, the solution was diluted with DMSO and purified by ISCO RP-HPLC. Upon lyophlization, (9H-fluoren-9-yl)methyl (5-((S)-2-((S)-2-(2-azidoacetamido)-3-methylbutanamido)-5-ureidopentanamido)-2-(hydroxymethyl)benzyl)(methyl)carbamate (220 mg, 49% yield was obtained. LC/MS: MH⁺=728.4, Rt=0.96 min (2 min acidic method).

### Synthesis of (9H-fluoren-9-yl)methyl (5-((S)-2-((S)-2-(2-azidoacetamido)-3-methylbutanamido)-5-ureidopentanamido)-2-(chloromethyl)benzyl)(methyl)carbamate

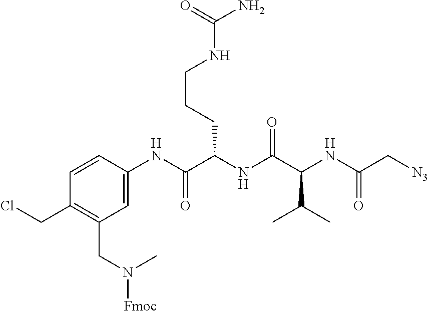

To a slurry of (9H-fluoren-9-yl)methyl (5-((S)-2-((S)-2-(2-azidoacetamido)-3-methylbutanamido)-5-ureidopentanamido)-2-(hydroxymethyl)benzyl)(methyl)carbamate (220 mg, 302 μmol) in CH₂Cl₂ (15 mL) at 0° C. was added thionyl chloride (110 μL, 1510 μmol). After stirring for 2 hours, the volatiles were removed in vacuo and the residue was triturated with Et2O. The material was used as is for next step. LC/MS: MH⁺=746.4, Rt=1.13 min (2 min acidic method).

### Synthesis of (9H-fluoren-9-yl)methyl (5-((S)-2-((S)-2-(((2-azidoethoxy)carbonyl)amino)-3-methylbutanamido)-5-ureidopentanamido)-2-(hydroxymethyl)benzyl)(methyl)carbamate

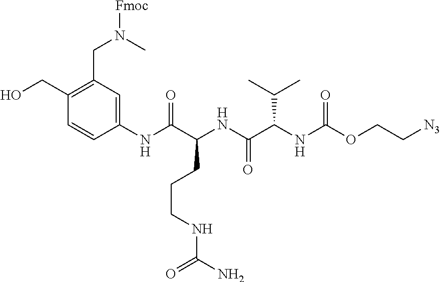

To a solution of (9H-fluoren-9-yl)methyl (5-((S)-2-((S)-2-amino-3-methylbutanamido)-5-ureidopentanamido)-2-(hydroxymethyl)benzyl)(methyl)carbamate TFA salt (1.65 g, 1.89 mmol) and 2-azidoethyl (4-nitrophenyl) carbonate (1.07 g, 4.25 mmol) in DMF (9.5 mL) was added DIEA (986 μL, 5.66 mmol). After stirring for 2 hours, the solution was partitioned between EtOAc and NH4Cl(sat.), washed with brine, dried over MgSO₄, filtered, concentrated and purified by SiO₂ chromatography (0-30% MeOH/CH₂Cl₂ eluant). After concentration, (9H-fluoren-9-yl)methyl (5-((S)-2-((S)-2-(((2-azidoethoxy)carbonyl)amino)-3-methylbutanamido)-5-ureidopentanamido)-2-(hydroxymethyl)benzyl)(methyl)carbamate (735 mg, 51% yield) was obtained. LC/MS: MH⁺=758.7, Rt=2.20 min (5 min acidic method).

### Synthesis of (9H-fluoren-9-yl)methyl (5-((S)-2-((S)-2-(((2-azidoethoxy)carbonyl)amino)-3-methylbutanamido)-5-ureidopentanamido)-2-(chloromethyl)benzyl)(methyl)carbamate

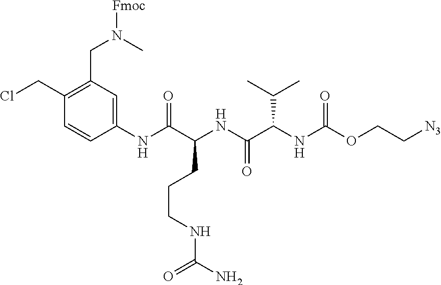

To a solution of (9H-fluoren-9-yl)methyl (5-((S)-2-((S)-2-(((2-azidoethoxy)carbonyl)amino)-3-methylbutanamido)-5-ureidopentanamido)-2-(hydroxymethyl)benzyl)(methyl)carbamate (735 mg, 970 μmol) and NaHCO₃ (570 mg, 6790 μmol) in THF (10 mL) at 0° C. was added thionyl chloride (283 μL, 3880 μmol). After stirring for 1 hour, the solution was partitioned between EtOAc and NaHCO₃(sat.), extracted further with EtOAc, dried over MgSO₄, filtered, concentrated, purified by SiO₂ chromatography (0−1=20% iPrOH/CH₂Cl₂ eluant). After concentration, (9H-fluoren-9-yl)methyl (5-((S)-2-((S)-2-(((2-azidoethoxy)carbonyl)amino)-3-methylbutanamido)-5-ureidopentanamido)-2-(chloromethyl)benzyl)(methyl)carbamate (578 mg, 77% yield) was obtained. LC/MS: MH⁺=776.6, Rt=2.66 min (5 min acidic method).

### Synthesis of (9H-fluoren-9-yl)methyl (S)-(5-(2-aminopropanamido)-2-(hydroxymethyl)benzyl)(methyl)carbamate

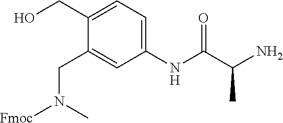

Boc-Ala-OH (0.724 g, 3.83 mmol), EDC·HCl (0.734 g, 3.83 mmol) and HOAT (0.521 g, 3.83 mmol) were dissolved in DMF (3 mL). The reaction was stirred at RT for 10 min at which time (9H-fluoren-9-yl)methyl (5-amino-2-(((tert-butyldiphenylsilyl)oxy)methyl)benzyl)(methyl)carbamate (2 g, 3.19 mmol) was added. The reaction was stirred at RT for 10 min. The reaction mixture was added dropwise to roughly 250 mL of water under vigorous stirring. A precipitate was formed but it was very sticky. The solution was stirred at RT for 30 min and sonicated for 5 min. The precipitate was collected by vacuum filtration. The cake was washed with water (100 mL). The product was air dried under vacuum to obtain the desired amide product (2.36 grams, 93% yield). The amide product was treated with 25% TFA/CH₂Cl₂ (5.33 mL) for one hour at which time the volatiles were removed in vacuo. To clip the TFA ester that has partially formed, the residue was dissolved in MeOH (10 mL) and dripped dropwise into 7N ammonia in MeOH (15.32 mL, 107 mmol). The volatiles were removed in vacuo, the residue was triturated with Et₂O and the pumped on to yield (9H-fluoren-9-yl)methyl (S)-(5-(2-aminopropanamido)-2-(hydroxyethyl)benzyl)(methyl)carbamate (3.08 grams, 94% yield). LC/MS: MH⁺=460.5, Rt=1.49 min (5 min acidic method). The material was used as is for next step.

### Synthesis of (9H-fluoren-9-yl)methyl (5-((S)-2-((S)-2-amino-3-methylbutanamido)propanamido)-2-(hydroxymethyl)benzyl)(methyl)carbamate

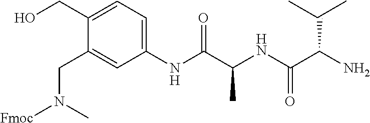

Boc-Val-OH (0.567 g, 2.176 mmol), EDC·HCl (0.501 g, 2.61 mmol) and HOAT (0.355 g, 2.61 mmol) were dissolved in DMF (4 mL). The reaction was stirred at RT for 10 min at which time (9H-fluoren-9-yl)methyl (S)-(5-(2-aminopropanamido)-2-(hydroxymethyl)benzyl)(methyl)carbamate (1 g, 2.176 mmol) was added. The reaction was stirred at RT for 10 min. The reaction mixture was added dropwise to roughly 1 L of water under vigorous stirring. The solution was stirred at RT for 30 min and sonicated for 5 min. The precipitate was collected by vacuum filtration. The cake was washed with water (100 mL). The product was air dried under vacuum to obtain the desired crude amide product (1.6 grams). The amide product (1.06 g, 1.61 mmol) was treated with 25% TFA/CH₂Cl₂ (5.33 mL) for one hour at which time the volatiles were removed in vacuo. To clip the TFA ester that has partially formed, the residue was dissolved in MeOH (10 mL) and dripped dropwise into 7N ammonia in MeOH (15.32 mL, 107 mmol). The volatiles were removed in vacuo, the residue was dissolved in DMSO and purified by RP-HPLC. Upon lyophilization, (9H-fluoren-9-yl)methyl (5-—((S)-2-((S)-2-amino-3-methylbutanamido)propanamido)-2-(hydroxymethyl)benzyl)(methyl)carbamate trifluoroacetate (300 mg, 33% yield) was obtained. LC/MS: MH⁺=559.6, Rt=1.58 min (5 min acidic method).

### Synthesis of (9H-fluoren-9-yl)methyl (5-((S)-2-((S)-2-(((2-azidoethoxy)carbonyl)amino)-3-methylbutanamido)propanamido)-2-(hydroxymethyl)benzyl)(methyl)carbamate

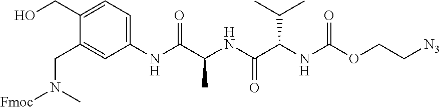

To a solution of (9H-fluoren-9-yl)methyl (5-—((S)-2-((S)-2-amino-3-methylbutanamido)propanamido)-2-(hydroxymethyl)benzyl)(methyl)carbamate trifluoroacetate (150 mg, 223 μmol) and 2-azidoethyl (4-nitrophenyl) carbonate (112 mg, 446 μmol) in DMF (1 mL) was added DIEA 1194 μL, 1115 μmol). After stirring for 1 hour, the solution was diluted with DMSO and purified by RP-HPLC. Upon lyophilization, (9H-fluoren-9-yl)methyl (5-((S)-2-((S)-2-(((2-azidoethoxy)carbonyl)amino)-3-methylbutanamido)propanamido)-2-(hydroxymethyl)benzyl)(methyl)carbamate (185 mg, ca. 100% yield) was obtained. LC/MS: (M−H₂O)+H⁺=654.6, Rt=2.45 (5 min acidic method).

### Synthesis of (9H-fluoren-9-yl)methyl (5-((S)-2-((S)-2-(((2-azidoethoxy)carbonyl)amino)-3-methylbutanamido)propanamido)-2-(chloromethyl)benzyl)(methyl)carbamate

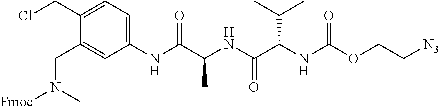

To a mixture of (9H-fluoren-9-yl)methyl (5-((S)-2-((S)-2-(((2-azidoethoxy)carbonyl)amino)-3-methylbutanamido)propanamido)-2-(hydroxymethyl)benzyl)(methyl)carbamate (185 mg, 235 μmol) and sodium bicarbonate (138 mg, 1648 μmol) in THF (7 mL) in an ice bath was added thionyl chloride (69 μL, 942 μmol). After stirring for 30 minutes, the solution was added dropwise into NaHCO₃(sat.) (100 mL) and was then extracted with EtOAc (3×50 mL). The combined organics were washed with NaCl(sat.), dried over MgSO₄, filtered, concentrated and purified by SiO₂ chromatography (0-20% iPrOH/CH₂Cl₂ eluant) to yield (9H-fluoren-9-yl)methyl (5-—((S)-2-((S)-2-(((2-azidoethoxy)carbonyl)amino)-3-methylbutanamido)propanamido)-2-(chloromethyl)benzyl)(methyl)carbamate (115 mg, 71% yield). LC/MS: MH⁺=690.6, Rt=2.96 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxopropan-2-yl)amino)-3-methyl-1-oxobutan-2-yl)amino)-6-oxohexane-1,5-diyl)dicarbamate

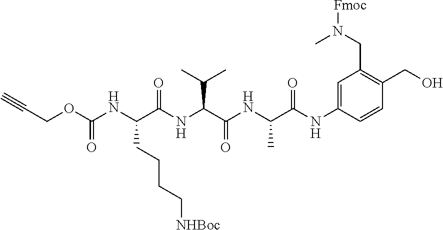

To a solution of N6-(tert-butoxycarbonyl)-N2-((prop-2-yn-1-yloxy)carbonyl)-L-lysine (88 mg, 268 μmol), HOAT (36.4 mg, 268 μmol) and EDC (51.3 mg, 268 μmol) in DMF (2 mL) was stirred for 10 minutes at which time DIEA (117 μL, 669 μmol) and (9H-fluoren-9-yl)methyl (5-((S)-2-((S)-2-amino-3-methylbutanamido)propanamido)-2-(hydroxymethyl)benzyl)(methyl)carbamate TFA salt (150 mg, 223 μmol) were added. After stirring for 20 minutes, the solution was diluted with DMSO and purified by RP-HPLC. Upon lyophilization, tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxopropan-2-yl)amino)-3-methyl-1-oxobutan-2-yl)amino)-6-oxohexane-1,5-diyl)dicarbamate (162 mg, 74% yield) was obtained. LC/MS: MH⁺=869.8, Rt=2.66 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxopropan-2-yl)amino)-3-methyl-1-oxobutan-2-yl)amino)-6-oxohexane-1,5-diyl)dicarbamate

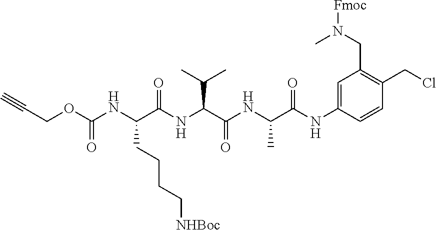

To a mixture of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxopropan-2-yl)amino)-3-methyl-1-oxobutan-2-yl)amino)-6-oxohexane-1,5-diyl)dicarbamate (162 mg, 165 μmol) and sodium bicarbonate (97 mg, 1154 μmol) in THF (7 mL) in an ice bath was added thionyl chloride (48 μL, 659 μmol). After stirring for 30 minutes, the solution was added dropwise into NaHCO₃(sat.) (100 mL) and was then extracted with EtOAc (3×50 mL). The combined organics were washed with NaCl(sat.), dried over MgSO₄, filtered, concentrated and purified by SiO₂ chromatography (0-20% iPrOH/CH₂Cl₂ eluant) to yield tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxopropan-2-yl)amino)-3-methyl-1-oxobutan-2-yl)amino)-6-oxohexane-1,5-diyl)dicarbamate (115 mg, 71% yield). LC/MS: MH⁺=887.8, Rt=3.10 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxo-5-ureidopentan-2-yl)amino)-3-methyl-1-oxobutan-2-yl)amino)-6-oxohexane-1,5-diyl)dicarbamate

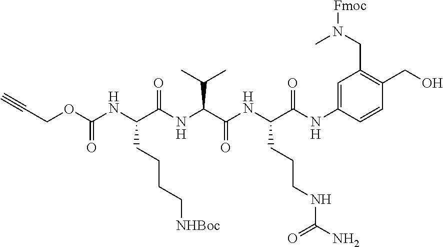

To a solution of N6-(tert-butoxycarbonyl)-N2-((prop-2-yn-1-yloxy)carbonyl)-L-lysine (1.787 g, 5.44 mmol) and HATU (1.88 g, 4.95 mmol) in DMF (20 mL) was added DIEA (862 μL, 4.95 mmol). After stirring for 5 minutes, (9H-fluoren-9-yl)methyl (5-((S)-2-((S)-2-amino-3-methylbutanamido)-5-ureidopentanamido)-2-(hydroxymethyl)benzyl)(methyl)carbamate (3.19 g, 4.95 mmol) in DMF (10 mL) was added. After stirring for an additional 2 hours, the solution dripped into 1 L of H₂O. The resulting solid was collected by filtration, dissolved in CH₂Cl₂/MeOH and purified by SiO₂ chromatography to yield tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxo-5-ureidopentan-2-yl)amino)-3-methyl-1-oxobutan-2-yl)amino)-6-oxohexane-1,5-diyl)dicarbamate (900 mg, 19% yield). LC/MS: MH⁺=955.9, Rt=2.55 min (5 min acidic method).

### Synthesis of tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)amino)-1-oxo-5-ureidopentan-2-yl)amino)-3-methyl-1-oxobutan-2-yl)amino)-6-oxohexane-1,5-diyl)dicarbamate

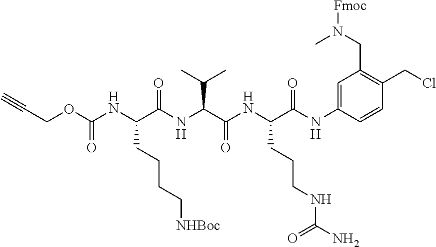

To a mixture of tert-butyl prop-2-yn-1-yl ((S)-6-(((S)-1-(((S)-1-((3-(((((9H-fluoren-9-yl)methoxy)carbonyl)(methyl)amino)methyl)-4-(hydroxymethyl)phenyl)amino)-1-oxo-5-ureidopentan-2-yl)amino)-3-methyl-1-oxobutan-2-yl)amino)-6-oxohexane-1,5-diyl)dicarbamate (507 mg, 531 μmol) in MeCN (8 mL) in an ice bath was added thionyl chloride (97 μL, 1327 μmol). After stirring for 60 minutes, the volatiles were removed in vacuo, the residue was dissolved in CH₂Cl₂ (20 mL)+MeOH (1 mL) and Boc2O (246 mg, 1062 μmol) and DIEA (556 μL, 3180 μmol) were added. After stirring for 60 minutes, the volatiles were removed in vacuo, the residue was dissolved in CH₂Cl₂ and was purified by SiO₂ chromatography (0-30% MeOH/CH₂Cl₂) to yield tert-butyl prop-2-yn-1-yl ((S)-6-(((S)-1-(((S)-1-((3-(((((9H-fluoren-9-yl)methoxy)carbonyl)(methyl)amino)methyl)-4-(chloromethyl)phenyl)ammino)-1-oxo-5-ureidopentan-2-yl)amino)-3-methyl-1-oxobutan-2-yl)amino)-6-oxohexane-1,5-diyl)dicarbamate (342 mg, 66% yield). LC/MS: MH⁺=973.9, Rt=1.21 min (2 min acidic method).

### Synthesis of (S)-5-(3-(tert-butoxy)-3-oxopropyl)-1-(9H-fluoren-9-yl)-3,6-dioxo-2,10,13,16,19,22-hexaoxa-4,7-diazapentacosan-25-oic acid

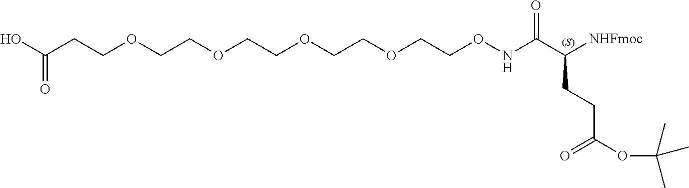

To the solution of Fmoc-Glu(OTBu)-OSu (200 mg, 0.383 mmol) in DMF (1 ml) was added the solution of Amino-PEG6-Acid (271 mg, 0.765 mmol) in MeOH (1 ml), then was followed by DIPEA (267 μl, 1.531 mmol). The resulting solution was stirred at RT for 30 min. The crude mixture was purified by C18 column (100 g cartridge, MeCN/Water with 0.05% TFA 0-100% over 16 CV) to obtain (S)-5-(3-(tert-butoxy)-3-oxopropyl)-1-(9H-fluoren-9-yl)-3,6-dioxo-2,10,13,16,19,22-hexaoxa-4,7-diazapentacosan-25-oic acid (273 mg, 100% yield). LC/MS: MH⁺=717.5, Rt=1.12 min (3 min acidic run).

### Synthesis of (S)-21-((((9H-fluoren-9-yl)methoxy)carbonyl)amino)-20-oxo-4,7,10,13,16-pentaoxa-19-azatetracosanedioic acid

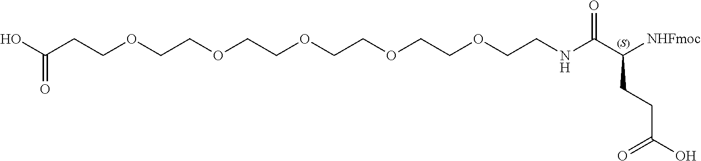

To the pre-cooled (S)-5-(3-(tert-butoxy)-3-oxopropyl)-1-(9H-fluoren-9-yl)-3,6-dioxo-2,10,13,16,19,22-hexaoxa-4,7-diazapentacosan-25-oic acid (273 mg, 0.381 mmol) and triethylsilane (0.061 mL, 0.381 mmol) was added pre-cooled TFA (25% in DCM, v/v) (11.700 mL, 38.0 mmol) at 0° C. in ice-water bath. mixture was stirred at 0° C. for 15 min, then raised to RT, and stirred at RT for 40 min. The crude mixture was concentrated under high vacuum and then dried over high vacuum overnight, and then purified by C18 column (50 g cartridge, MeCN/Water with 0.05% TFA 0-100% over 16 CV) to obtain (S)-21-((((9H-fluoren-9-yl)methoxy)carbonyl)amino)-20-oxo-4,7,10,13,16-pentaoxa-19-azatetracosanedioic acid (196 mg, 78% yield). LC/MS MH⁺=661.3, Rt=0.91 min (3 min acidic run).

### Synthesis of (S)-5-(3-(tert-butoxy)-3-oxopropyl)-1-(9H-fluoren-9-yl)-3,6-dioxo-2,10,13,16,19,22,25-heptaoxa-4,7-diazaoctacosan-28-oic acid

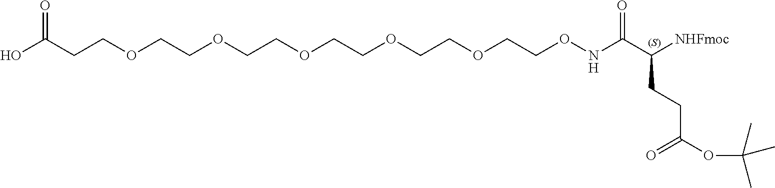

To the solution of Fmoc-Glu(OTBu)-OSu (50 mg, 0.096 mmol) in DMF (0.5 ml) was added the solution of Amino-PEG6-Acid (67.6 mg, 0.191 mmol) in MeOH (1 ml), then was followed by DIPEA (66.8 μl, 0.383 mmol). The resulting solution was stirred at RT for 30 min. The crude mixture was purified by C-18 Column (100 g cartridge, MeCN/Water with 0.05% TFA 0-100% over 16 CV) to obtain (S)-5-(3-(tert-butoxy)-3-oxopropyl)-1-(9H-fluoren-9-yl)-3,6-dioxo-2,10,13,16,19,22,25-heptaoxa-4,7-diazaoctacosan-28-oic acid (44 mg, 60% yield). LC/MS MH⁺=761.5, Rt=1.12 min (3 min acidic run).

### Synthesis of (S)-24-((((9H-fluoren-9-yl)methoxy)carbonyl)amino)-23-oxo-4,7,10,13,16,19-hexaoxa-22-azaheptacosanedioic acid

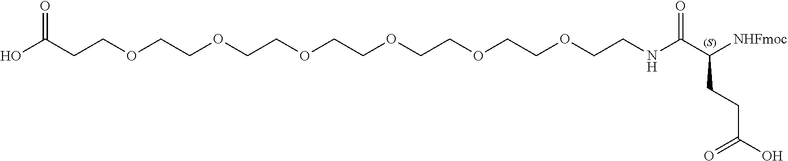

To the pre-cooled (S)-5-(3-(tert-butoxy)-3-oxopropyl)-1-(9H-fluoren-9-yl)-3,6-dioxo-2,10,13,16,19,22,25-heptaoxa-4,7-diazaoctacosan-28-oic acid (43.9 mg, 0.058 mmol) and triethylsilane (9.22 μl, 0.058 mmol) was added pre-cooled TFA (25% in DCM, v/v) (1778 μl, 5.77 mmol) at 0° C. in ice-water bath. The reaction mixture was stirred at 0° C. for 15 min, then raised to RT, and stirred at RT for 40 min. The crude mixture was concentrated under high vacuum, then dried over high vacuum overnight, and purified by C18 column (50 g cartridge, MeCN/Water 0-100% with 0.05% TFA over 16 CV) to obtain (S)-24-((((9H-fluoren-9-yl)methoxy)carbonyl)amino)-23-oxo-4,7,10,13,16,19-hexaoxa-22-azaheptacosanedioic acid (16 mg, 39% yield). LC/MS MH⁺=705.4, Rt=0.91 min (3 min acidic run).

### Synthesis of di-tert-butyl (azanediylbis(ethane-2,1-diyl))bis(methylcarbamate)

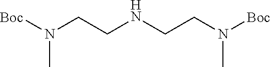 [SMILES: CN(CCNCCN(C)C(=O)OC(C)(C)C)C(=O)OC(C)(C)C]

In a 40 mL glass vial equipped with magnetic stir bar, palladium on carbon (18.32 mg, 0.172 mmol) was dissolved in EtOH (Volume: 5 mL). tert-butyl (2-aminoethyl)(methyl)carbamate (0.308 mL, 1.722 mmol) and tert-butyl methyl(2-oxoethyl)carbamate (0.298 mL, 1.722 mmol) were added. The reaction atmosphere was switched to hydrogen (treat reaction chamber with vacuum and refill with nitrogen, repeat 4×. Then treat reaction chamber with vacuum and refill with hydrogen, repeat 4×). The reaction was stirred at RT for 16 hr. The reaction was analyzed by LC-MS and the desired product was found (LC-MS crude: product ret. time: 1.45 min, [M]+: 332.3). The reaction atmosphere was switched to nitrogen. The reaction solution was filtered through a celite pad. The pad was then washed with EtOAc (20 mL). The combined organic layer was removed under reduced pressure. The product was then dried under high vacuum for 30 min. The product di-tert-butyl (azanediylbis(ethane-2,1-diyl))bis(methylcarbamate) was used as is in next step assuming 172 μmol.

### Synthesis of tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate

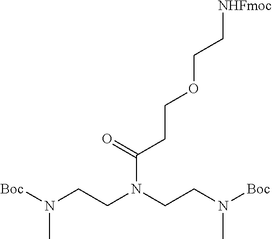

3-(2-((((9H-fluoren-9-yl)methoxy)carbonyl)amino)ethoxy)propanoic acid (367 mg, 1.034 mmol) and HATU (393 mg, 1.034 mmol) were dissolved in DMF (8 mL). DIPEA (0.542 mL, 3.10 mmol) was added. The reaction was stirred at RT for 10 min. The mixture was added to di-tert-butyl (azanediylbis(ethane-2,1-diyl))bis(methylcarbamate) (172 μmol). After stirring for 30 minutes, the reaction mixture was poured into aq. sat. ammonium chloride solution (100 mL). It was extracted with EtOAc (2×40 mL). The combined organic layer was washed with brine, treated with MgSO₄, filtered and removed under reduced pressure. The product was then purified by SiO₂ chromatography with EtOAc/n-heptanes eluant. Upon removal of volatiles, tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate was obtained. LC/MS: MH⁺=669.3, Rt=3.03 min (5 min acidic run).

### Synthesis of tert-butyl (1-(9H-fluoren-9-yl)-11-(2-(methylamino)ethyl)-3,10-dioxo-2,7-dioxa-4,11-diazatridecan-13-yl)(methyl)carbamate trifluoroacetate

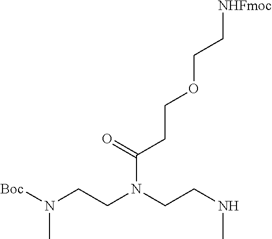

To a solution of tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate (647 mg, 967 μmol) in CH₂Cl₂ (4 mL) was added TFA (75 μL, 967 μL). After standing at rt for 14 hours, the volatiles were removed in vacuo, the residue was dissolved in DMSO and purified by ISCO RP-HPLC. Upon lyophilization, tert-butyl (1-(9H-fluoren-9-yl)-11-(2-(methylamino)ethyl)-3,10-dioxo-2,7-dioxa-4,11-diazatridecan-13-yl)(methyl)carbamate trifluoroacetate (110 mg, 20%) was obtained. LC/MS: MH⁺=569.6, Rt=0.89 min (2 min acidic run).

### Synthesis of tert-butyl 11-(2-((tert-butoxycarbonyl)(methyl)amino)ethyl)-1-(9H-fluoren-9-yl)-14-methyl-3,10,15-trioxo-2,7,18,21-tetraoxa-4,11,14-triazatetracosan-24-oate

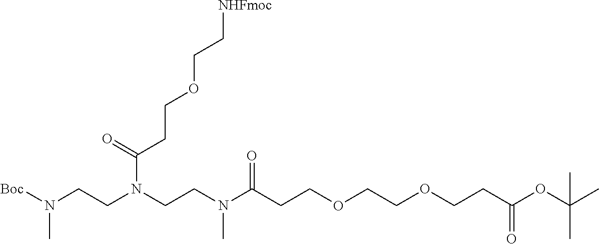

Acid-PEG2-tertbuyl ester (106 mg, 0.405 mmol) and HATU (147 mg, 0.387 mmol) were combined in DMF (2 mL). DIPEA (0.322 mL, 1.841 mmol) was added. It was left at RT for 5 min. tert-butyl (1-(9H-fluoren-9-yl)-11-(2-(methylamino)ethyl)-3,10-dioxo-2,7-dioxa-4,11-diazatridecan-13-yl)(methyl)carbamate TFA salt (251 mg, 0.368 mmol) was added. The reaction was left at RT for 5 min. The reaction was diluted with DMSO and purified by ISCO RP-HPLC. Upon lyophilization, tert-butyl 11-(2-((tert-butoxycarbonyl)(methyl)amino)ethyl)-1-(9H-fluoren-9-yl)-14-methyl-3,10,15-trioxo-2,7,18,21-tetraoxa-4,11,14-triazatetracosan-24-oate (199 mg, 67%) was obtained. LC/MS: MH⁺=813.9, Rt=1.24 min (2 min acidic run).

### Synthesis of 1-(9H-fluoren-9-yl)-14-methyl-11-(2-(methylamino)ethyl)-3,10,15-trioxo-2,7,18,21-tetraoxa-4,11,14-triazatetracosan-24-oic acid

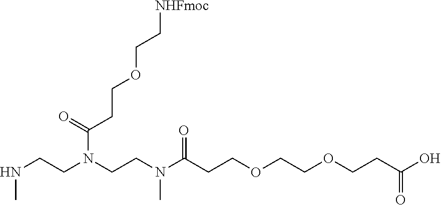

tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate (199 mg, 245 μmol) was treated with 2:1 CH₂Cl₂/TFA (3 mL) for 1 hour at which the volatiles were removed in vacuo. The residue was dissolved in DMSO and purified by ISCO RP-HPLC. Upon lyophilization, 1-(9H-fluoren-9-yl)-14-methyl-11-(2-(methylamino)ethyl)-3,10,15-trioxo-2,7,18,21-tetraoxa-4,11,14-triazatetracosan-24-oic acid (166 mg, 88% yield) was obtained as the TFA salt. LC/MS MH⁺=656.3, Rt=0.76 min (2 min acidic run).

### Synthesis of (9H-fluoren-9-yl)methyl (2-(3-(bis(2-(methylamino)ethyl)amino)-3-oxopropoxy)ethyl)carbamate trifluoroacetate

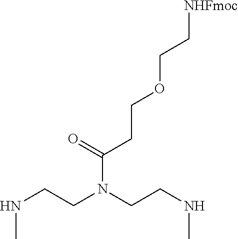

tert-butyl (11-(2-((tert-butoxycarbonyl)(methyl)amino)ethyl)-1-(9H-fluoren-9-yl)-3,10-dioxo-2,7-dioxa-4,11-diazatridecan-13-yl)(methyl)carbamate (614 mg, 918 μmol) was treated with 25% TFA/CH₂Cl₂ (8 mL) for one hour at which time the volatiles were removed in vacuo. The residue was dissolved in MeCN/H₂O and lyophilized to yield (9H-fluoren-9-yl)methyl (2-(3-(bis(2-(methylamino)ethyl)amino)-3-oxopropoxy)ethyl)carbamate trifluoroacetate. LC/MS: MH⁺=469.1, Rt=1.05 min (5 min acidic method).

### Synthesis of tert-butyl 1-(9H-fluoren-9-yl)-14-methyl-11-(2-(methylamino)ethyl)-3,10,15-trioxo-2,7,18,21-tetraoxa-4,11,14-triazatetracosan-24-oate trifluoroacetate

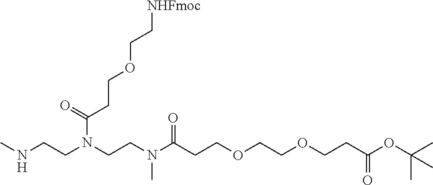

To a solution of 3-(2-(3-(tert-butoxy)-3-oxopropoxy)ethoxy)propanoic acid (40 mg, 152 μmol) and HATU (69.6 mg, 183 μmol) in DMF (2 mL) was added DIEA (160 μL, 915 μmol). After stirring for 5 minutes, (9H-fluoren-9-yl)methyl (2-(3-(bis(2-(methylamino)ethyl)amino)-3-oxopropoxy)ethyl)carbamate trifluoroacetate (425 mg, 610 μmol) was added. After stirring for an additional 30 minutes, the solution was diluted with DMSO and purified by ISCO RP-HPLC. Upon lyophilization, tert-butyl 1-(9H-fluoren-9-yl)-14-methyl-11-(2-(methylamino)ethyl)-3,10,15-trioxo-2,7,18,21-tetraoxa-4,11,14-triazatetracosan-24-oate trifluoroacetate (26.1 mg, 21% yield) was obtained. LC/MS: MH⁺=713.6, Rt=2.09 min (5 min acidic method).

### Synthesis of di-tert-butyl 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioate

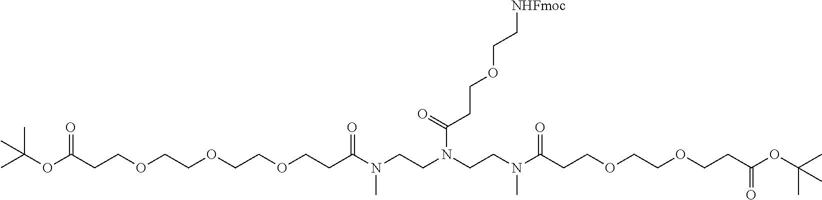

To a solution of 2,2-dimethyl-4-oxo-3,7,10,13-tetraoxahexadecan-16-oic acid (11.6 mg, 38 μmol) and HATU (12 mg, 32 μmol) in DMF (2 mL) was added DIEA (44 μL, 253 μmol). After stirring for 5 minutes, tert-butyl 1-(9H-fluoren-9-yl)-14-methyl-11-(2-(methylamino)ethyl)-3,10,15-trioxo-2,7,18,21-tetraoxa-4,11,14-triazatetracosan-24-oate trifluoroacetate (26.1 mg, 32 μmol) was added. After stirring for an additional 30 minutes, the solution was diluted with DMSO and purified by ISCO RP-HPLC. Upon lyophilization, di-tert-butyl 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioate (21.4 mg, 68% yield) was obtained. LC/MS: MH⁺=1001.7, Rt=1.95 min (5 min acidic method).

### Synthesis of 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioic acid

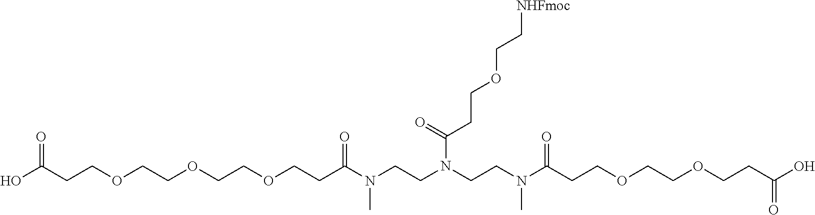

di-tert-butyl 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioate (21.4 mg, 21 μmol) was treated with 50% TFA/CH₂Cl₂ (2 mL) for one hour at which time the volatiles were removed in vacuo. The residue was dissolved in MeCN/H₂O and lyophilized to yield 17-(3-(2-((((9H-fluoren-9-yl)methoxy)carbonyl)amino)ethoxy)propanoyl)-14,20-dimethyl-13,21-dioxo-4,7,10,24,27-pentaoxa-14,17,20-triazatriacontanedioic acid (20.2 mg, 99% yield). LC/MS: MH⁺=889.6, Rt=1.94 min (5 min acidic method).

### Synthesis of 16-(3-(2-((tert-butoxycarbonyl)amino)ethoxy)propanoyl)-4,7,10,13,19,22,25,28-octaoxa-16-azahentriacontanedioic acid

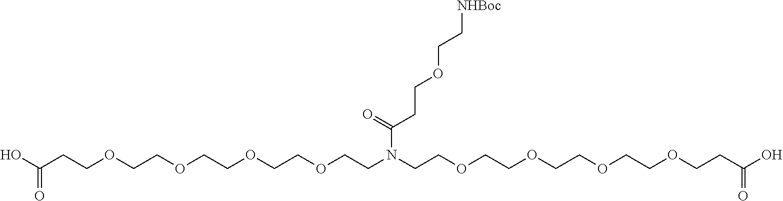 [SMILES: CC(C)(C)OC(=O)NCCOCCC(=O)N(CCOCCOCCOCCOCCC(=O)O)CCOCCOCCOCCOCCC(=O)O]

A mixture of 3-(2-((tert-butoxycarbonyl)amino)ethoxy)propanoic acid (30 mg, 0.13 mmol), HATU (42 mg, 0.11 mmol), and DIPEA (79 μL, 0.46 mmol) in DMF (1 mL) was stirred at RT for 40 min. This solution was added to 4,7,10,13,19,22,25,28-octaoxa-16-azahentriacontanedioic acid·HCl salt (from BroadPharm) (50 mg, 0.091 mmol). More DIPEA (0.048 mL, 0.27 mmol) was added. The mixture was stirred at RT for 2.5 h, diluted with DMSO (1 mL) and loaded onto RP-C18 ISCO column (50 g, gold). The column was eluted with MeCN-water (0.1% TFA modifier). Fractions containing the desired product were combined and lyophilized to give the product as an oil (29.7 mg, 45% yield). LC/MS: MH⁺=729.3, Rt=0.77 min (2 min acidic method).

### Synthesis of 1-(2,5-dioxopyrrolidin-1-yl) 5-methyl (tert-butoxycarbonyl)-L-glutamate

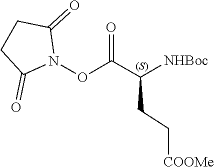

To a stirred solution of Boc-Glu(OMe)-OH (2.5 g, 9.57 mmol) and HOSu (1.211 g, 10.53 mmol) in THF (dry, 25 ml) at 0° C. in ice-water bath, was added DCC (2.073 g, 10.05 mmol) in portions. After 15 min, the reaction mixture was allowed to warm to room temperature and was stirred overnight. The solid DCU byproduct was filtered off. The filtrate was concentrated. The resulting thick oil was dissolved in DCM (40 ml), and was allowed to stand for 1 h and then was filtered to remove more DCU. The filtrate was evaporated, and the resulting glassy material was dried under high vacuum for 3 h to obtain 1-(2,5-dioxopyrrolidin-1-yl) 5-methyl (tert-butoxycarbonyl)-L-glutamate (3.47 g solid foam that was carried on next reaction without further purification). LC/MS: M-Boc+1=259.2, Rt=0.81 min (3 min acidic run).

### Synthesis of (S)-6-(3-methoxy-3-oxopropyl)-2,2-dimethyl-4,7-dioxo-3,11,14,17,20,23,26-heptaoxa-5,8-diazanonacosan-29-oic acid

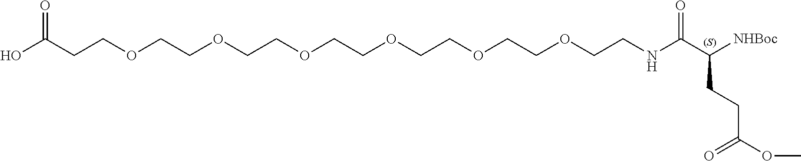

To the solution of 1-(2,5-dioxopyrrolidin-1-yl) 5-methyl (tert-butoxycarbonyl)-L-glutamate (117 mg, 0.326 mmol) in DMF (1 ml) was added the solution of Amino-PEG6-Acid (231 mg, 0.653 mmol) in MeOH (1 ml), then was followed by DIPEA (228 μl, 1.306 mmol). The resulting solution was stirred at Rt for 30 min. The crude mixture was purified by C18 column (50 g cartridge, MeCN/Water with 0.05% TFA 0-100% over 16 CV) to obtain (S)-6-(3-methoxy-3-oxopropyl)-2,2-dimethyl-4,7-dioxo-3,11,14,17,20,23,26-heptaoxa-5,8-diazanonacosan-29-oic acid (138 mg, 71% yield). LC/MS: MH⁺=597.5, Rt=0.75 min (3 min acidic run).

### Synthesis of ethyl 3,8-dioxo-1-phenyl-2,11,14-trioxa-4,7-diazaheptadecan-17-oate

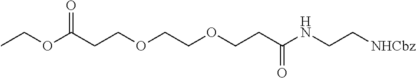 [SMILES: CCOC(=O)CCOCCOCCC(=O)NCCNC(=O)OCc1ccccc1]

A mixture of 3-(2-(3-ethoxy-3-oxopropoxy)ethoxy)propanoic acid (2.50 g, 10.7 mmol), benzyl (2-aminoethyl)carbamate (2.59 g, 11.2 mmol), HATU (4.46 g, 11.7 mmol), and DIPEA (10 mL, 57 mmol) in DMF (15 mL) was stirred at RT for 30 min. The mixture was concentrated to give the crude product, which was purified by RP-C18 ISCO column (150 g, gold) by elution with MeCN-water (0.1% TFA). Fractions containing the desired product contained impurity. The fractions were combined and concentrated to remove most of MeCN. The aq. layer was basified (pH 8-9) by addition of solid K₂CO₃, and the product was extracted with EtOAc. The combined EtOAc extract was dried over Na₂SO₄, filtered, and concentrated to give the crude product as an oil. As the product was still impure, this crude product was partitioned between EtOAc and 3M aq HCl. The combined organic extract was dried over Na₂SO₄, filtered and concentrated to give the title product in pure form as an oil (3.24 g, 74% yield). LC/MS: MH⁺=411.1, Rt=0.82 min (2 min acidic method).

### Synthesis of ethyl 2,2-dimethyl-4,12-dioxo-3,15,18-trioxa-5,8,11-triazahenicosan-21-oate

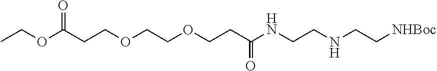 [SMILES: CCOC(=O)CCOCCOCCC(=O)NCCNCCNC(=O)OC(C)(C)C]

A mixture of ethyl 3,8-dioxo-1-phenyl-2,11,14-trioxa-4,7-diazaheptadecan-17-oate (3.24 g, 7.89 mmol), tert-butyl (2-oxoethyl)carbamate (1.257 g, 7.890 mmol), and Pd—C(10% wet) (250 mg, 2.35 mmol) in MeOH (20 mL) was hydrogenated at RT under balloon pressure for 3 days. The mixture was filtered through Celite and concentrated. The residual oil was diluted with DMSO and water. Insoluble material was removed by filtration. The filtrate was loaded onto RP-C18 ISCO column (150 g, gold). The column was eluted with MeCN-water (0.1% TFA). Fractions containing the desired product were combined and concentrated to give the product (TFA salt) as an oil (910 mg, 22% yield). LC/MS: MH⁺=420.5, Rt=0.56 min (2 min acidic method).

### Synthesis of 2,2-dimethyl-4,12-dioxo-3,15,18-trioxa-5,8,11-triazahenicosan-21-oic acid

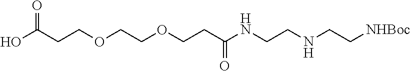 [SMILES: CC(C)(C)OC(=O)NCCNCCNC(=O)CCOCCOCCC(=O)O]

A mixture of ethyl 2,2-dimethyl-4,12-dioxo-3,15,18-trioxa-5,8,11-triazahenicosan-21-oate (910 mg, 2.17 mmol) and aq. LiOH (1M, 2.4 mL, 2.4 mmol) in MeOH (2.4 mL) and THF (2.4 mL) was heated at 50° C. for 0.5 h. LCMS analysis showed no reaction. The mixture was cooled to RT, and a solution of KOH (260 mg, 4.64 mmol) in water (1 mL) was added. The mixture was stirred at RT for 10 min. The mixture was concentrated, and the residue was taken up in water and loaded onto RP-C18 ISCO column (50 g, gold). The column was eluted with MeCN-water (0.1% TFA). Fractions containing the desired product were combined and concentrated to give the product (TFA salt) as a white solid (699 mg, 64% yield). LC/MS: MH⁺=392.5, Rt=0.56 min (2 min acidic method).

### Synthesis of 8-(((9H-fluoren-9-yl)methoxy)carbonyl)-2,2-dimethyl-4,12-dioxo-3,15,18-trioxa-5,8,11-triazahenicosan-21-oic acid

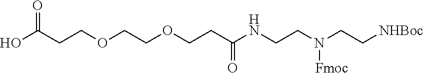

A mixture of 2,2-dimethyl-4,12-dioxo-3,15,18-trioxa-5,8,11-triazahenicosan-21-oic acid (699 mg, 1.79 mmol), (9H-fluoren-9-yl)methyl (2,5-dioxopyrrolidin-1-yl) carbonate (783 mg, 2.32 mmol), and DIPEA (1.00 mL, 5.73 mmol) in DMF (10 mL) was stirred at RT for 15 h. More (9H-fluoren-9-yl)methyl (2,5-dioxopyrrolidin-1-yl) carbonate (800 mg, 2.37 mmol) and DIPEA (0.50 mL, 2.9 mmol) were added. The mixture was stirred at RT for 1 h. The mixture was taken up in DMSO and loaded onto RP-C18 ISCO column (150 g, gold). The column was eluted with MeCN-water (0.1% TFA). Fractions containing the desired product were combined and concentrated to give the product as an oil (579 mg). LCMS analysis showed partial N-de-Boc of the product. The mixture was taken up in MeCN (5 mL) and treated with Boc2O (250 mg, 1.15 mmol) and DIPEA (1.00 mL, 5.73 mmol). The mixture was stirred at RT for 40 min and partitioned between EtOAc and 0.5 M aq. HCl. The combined organic extract was dried over Na₂SO₄ and concentrated to give the crude product (743 mg). This product was directly used in the next step without purification. LC/MS: MH⁺=614.5, Rt=1.06 min (2 min acidic method).

### Synthesis of allyl 8-(((9H-fluoren-9-yl)methoxy)carbonyl)-2,2-dimethyl-4,12-dioxo-3,15,18-trioxa-5,8,11-triazahenicosan-21-oate

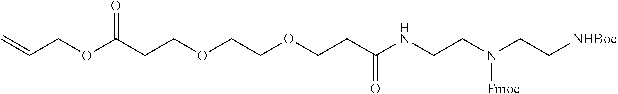

DMAP (0.012 g, 0.10 mmol) was added to a mixture of 8-(((9H-fluoren-9-yl)methoxy)carbonyl)-2,2-dimethyl-4,12-dioxo-3,15,18-trioxa-5,8,11-triazahenicosan-21-oic acid (743 mg), DCC (0.250 g, 1.21 mmol), and allyl alcohol (0.200 mL, 2.94 mmol) in THF (4 mL). The suspension was stirred at RT for 30 min (LCMS 1). The mixture was concentrated and purified by NP—ISCO (10-100% EtOAc in heptane, followed by 5% MeOH in EtOAc). Fractions containing the desired product were combined and concentrated to give a mixture of solid and oil. The mixture was triturated with ether and solid was removed by filtration. The ether layer was concentrated to give the title product as an oil (400 mg, 34% yield over the last 2 steps). LC/MS: MH⁺=654.7, Rt=1.16 min (2 min acidic method).

### Synthesis of allyl 4-(2-aminoethyl)-1-(9H-fluoren-9-yl)-3,8-dioxo-2,11,14-trioxa-4,7-diazaheptadecan-17-oate

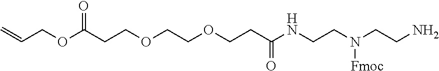

A mixture of allyl 8-(((9H-fluoren-9-yl)methoxy)carbonyl)-2,2-dimethyl-4,12-dioxo-3,15,18-trioxa-5,8,11-triazahenicosan-21-oate (400 mg, 0.612 mmol) and TFA (2 mL, 26.0 mmol) in DCM (4 mL) was stirred at RT for 20 min. The mixture was concentrated. The residue was taken up in MeCN and concentrated to remove as much TFA as possible to give the crude product, which was used in the next step without purification. LC/MS: MH⁺=554.6, Rt=0.75 min (2 min acidic method).

### Synthesis of 13,21,30-trioxo-4,7,10,24,27,31-hexaoxa-14,17,20-triazatetratriacont-33-enoic acid

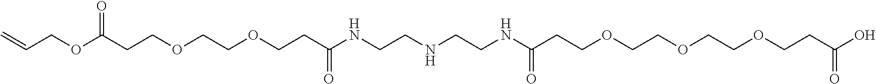 [SMILES: C=CCOC(=O)CCOCCOCCC(=O)NCCNCCNC(=O)CCOCCOCCOCCC(=O)O]

A mixture of allyl 4-(2-aminoethyl)-1-(9H-fluoren-9-yl)-3,8-dioxo-2,11,14-trioxa-4,7-diazaheptadecan-17-oate (crude from the previous step), 3-(2-(2-(3-((2,5-dioxopyrrolidin-1-yl)oxy)-3-oxopropoxy)ethoxy)ethoxy)propanoic acid (0.250 g, 0.720 mmol), and DIPEA (0.75 mL, 4.29 mmol) in DMF (3 mL) was stirred at RT for 40 min. Amidation was complete (LC/MS MH+786.8, Rt=0.96 min, 2 min acidic method). To this was then added dimethylamine in THF (2M, 3 mL, 6 mmol). The mixture was stirred at RT for 30 min. The mixture was concentrated and the residue was diluted with DMSO (5 mL), loaded onto RP-C18 ISCO column (100 g, gold), and eluted with MeCN-water (0.1% TFA). Fractions containing the desired product were combined and concentrated to give the title product (TFA salt) as an oil (355 mg, 86% yield over the last 2 steps). LC/MS: MH⁺=564.6, Rt=0.52 min (2 min acidic method).

### Synthesis of 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid

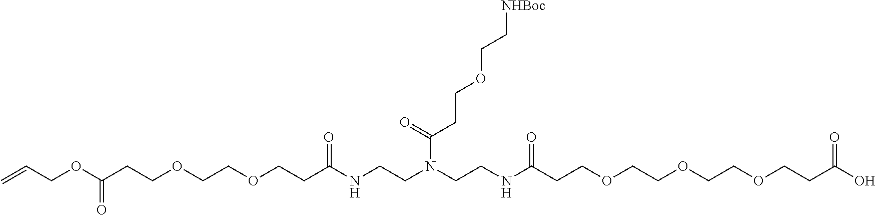 [SMILES: C=CCOC(=O)CCOCCOCCC(=O)NCCN(CCNC(=O)CCOCCOCCOCCC(=O)O)C(=O)CCOCCNC(=O)OC(C)(C)C]

A mixture of 13,21,30-trioxo-4,7,10,24,27,31-hexaoxa-14,17,20-triazatetratriacont-33-enoic acid (181 mg, 0.778 mmol), HATU (255 mg, 0.671 mmol), and DIPEA (450 μL, 2.58 mmol) in DMF (2 mL) was stirred at RT for 10 min. This was added to a DMF (1 mL) solution of 13,21,30-trioxo-4,7,10,24,27,31-hexaoxa-14,17,20-triazatetratriacont-33-enoic acid, trifluoroacetate (350 mg, 516 μmol). The mixture was stirred at RT for 10 min. The mixture was diluted with DMSO (3 mL) and loaded onto RP—ISCO column (100 g, gold). The column was eluted with MeCN-water (0.1% TFA). Fractions containing the desired product were combined and concentrated by ROTOVAP to give the product as an oil (340 mg). As the product contained ˜15% of N-de-Boc product, the product was taken up in MeCN (2 mL) and treated with Boc2O (40 mg, 0.184 mmol) and DIPEA (100 μL, 0.573 mmol). The mixture was stirred at RT for 30 min, concentrated, and the residue was taken up in DMSO and purified by RP—C18 ISCO column (50 g, gold)(MeCN-water 0.1% TFA). Fractions containing the desired product were combined and lyophilized to give the title product as an oil (236 mg, 59% yield). LC/MS: MH⁺=779.9, Rt=0.80 min (2 min acidic method).

### Synthesis of tert-butyl (2-((2-((((9H-fluoren-9-yl)methoxy)carbonyl)amino)ethyl)amino)ethyl)carbamate

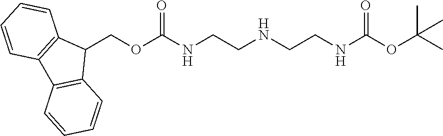 [SMILES: CC(C)(C)OC(=O)NCCNCCNC(=O)OCC1c2ccccc2-c2ccccc21]

In a 40 mL glass vial equipped with magnetic stir bar, palladium on carbon (0.067 g, 0.627 mmol), (9H-fluoren-9-yl)methyl (2-aminoethyl)carbamate (2 g, 6.27 mmol) and tert-butyl (2-oxoethyl)carbamate (0.999 g, 6.27 mmol) were dissolved in EtOH (16 mL). Acetic acid (0.036 mL, 0.627 mmol) and DIPEA (1.096 mL, 6.27 mmol) were added. The reaction atmosphere was switched to hydrogen (treat reaction chamber with vacuum and refill with nitrogen, repeat 4×.

Then treat reaction chamber with vacuum and refill with hydrogen, repeat 4×). The reaction was stirred at RT for 16 hr. The reaction solution was filtered through a celite pad. The pad was then washed with EtOAc (20 mL). The combined organic layer was removed under reduced pressure. The product was then dried under high vacuum for 30 min. The product was purified by reverse phase chromatography (Diluted with 4 mL DMSO, ISCO Gold C18 Column, 150 gr, Water/MeCN as eluent, 0.1% TFA as modifier). Fractions containing the desired product were combined and lyophilized to give the title product (TFA salt)(827 mg, 24% yield). LC/MS: MH⁺=426.4, Rt=0.86 (2 min acidic method).

### Synthesis of benzyl (2-((((9H-fluoren-9-yl)methoxy)carbonyl)amino)ethyl)(2-((tert-butoxycarbonyl)amino)ethyl)carbamate

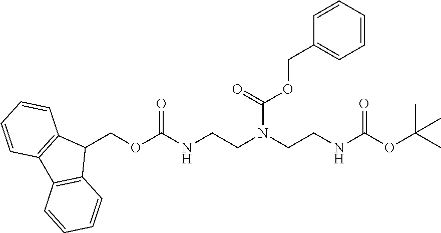 [SMILES: CC(C)(C)OC(=O)NCCN(CCNC(=O)OCC1c2ccccc2-c2ccccc21)C(=O)OCc1ccccc1]

Dissolved tert-butyl (2-((2-((((9H-fluoren-9-yl)methoxy)carbonyl)amino)ethyl)amino)ethyl)carbamate (827 mg, 1.533 mmol) in DCM (12 mL). Benzyl chloroformate (0.263 mL, 1.839 mmol) and DIPEA (0.803 mL, 4.60 mmol) were added. The reaction was stirred at RT for 1 hr. The reaction mixture was poured into aq. sat. ammonium chloride solution. The aqueous layer was extracted with EtOAc (2×). The combined organic layer was washed with brine, treated with MgSO₄, filtered and removed under reduced pressure. The product was purified by flash chromatography (ISCO Silica column, 120 gr, MeOH/DCM as eluent, 0% to 10%) to give the title compound (859 mg, quantitative yield). LC/MS: MH⁺(−Boc) 460.5, Rt=1.26 min (2 min acidic method).

### Synthesis of benzyl (2-((((9H-fluoren-9-yl)methoxy)carbonyl)amino)ethyl)(2-aminoethyl)carbamate

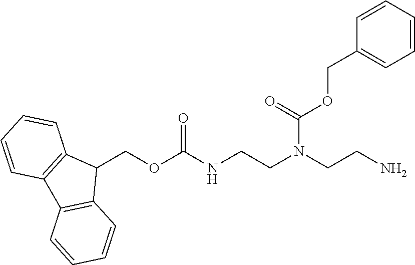 [SMILES: NCCN(CCNC(=O)OCC1c2ccccc2-c2ccccc21)C(=O)OCc1ccccc1]

In a 100 mL round bottom flask equipped with magnetic stir bar, benzyl (2-((((9H-fluoren-9-yl)methoxy)carbonyl)amino)ethyl)(2-((tert-butoxycarbonyl)amino)ethyl)carbamate (859 mg, 1.535 mmol) was dissolved in DCM (3 mL) and TFA (1 mL). The reaction was stirred at RT for 1 hr. DCE (30 mL) was added. The solvent was removed under reduced pressure to obtain the title product (TFA salt) (986 mg, 93% yield). The product was used as it is without further purification or characterization.

### Synthesis of ethyl 7-((benzyloxy)carbonyl)-1-(9H-fluoren-9-yl)-3,11-dioxo-2,14,17-trioxa-4,7,10-triazaicosan-20-oate

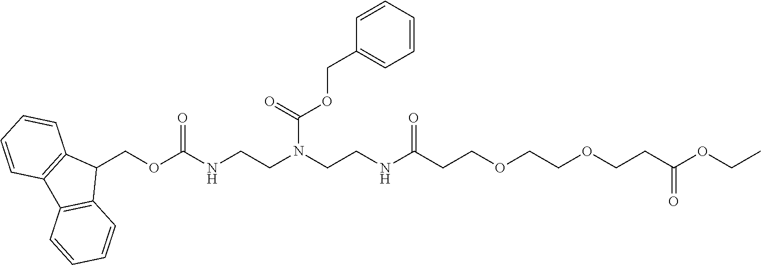 [SMILES: CCOC(=O)CCOCCOCCC(=O)NCCN(CCNC(=O)OCC1c2ccccc2-c2ccccc21)C(=O)OCc1ccccc1]

To a solution of 3-(2-(3-ethoxy-3-oxopropoxy)ethoxy)propanoic acid (230 mg, 0.982 mmol) in DMF (1.5 mL) was added HATU (299 mg, 0.785 mmol), DIPEA (0.572 mL, 3.27 mmol), and benzyl (2-((((9H-fluoren-9-yl)methoxy)carbonyl)amino)ethyl)(2-aminoethyl)carbamate (450 mg, 0.654 mmol). The mixture was stirred at RT for 1 h and concentrated. The residue was diluted with DMSO (3 mL) and purified by RP—ISCO chromatography (100 G ISCO Gold C18 Column, Water/MeCN as eluent, 0.1% TFA as modifier). The desired product fractions were combined and lyophilized to afford the title compound (206 mg, 44.2% yield). LC/MS: MH⁺=676, Rt=1.16 min (2 min acidic method).

### Synthesis of ethyl 4-(2-aminoethyl)-3,8-dioxo-1-phenyl-2,11,14-trioxa-4,7-diazaheptadecan-17-oate

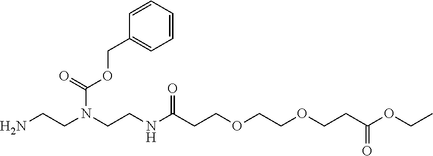 [SMILES: CCOC(=O)CCOCCOCCC(=O)NCCN(CCN)C(=O)OCc1ccccc1]

To a solution of ethyl 7-((benzyloxy)carbonyl)-1-(9H-fluoren-9-yl)-3,11-dioxo-2,14,17-trioxa-4,7,10-triazaicosan-20-oate (395 mg, 0.585 mmol) in EtOH (5 mL) was added dimethylamine in THF (1M, 2.92 mL, 2.92 mmol). The mixture was stirred at RT for 3 h and concentrated. The residue was diluted with DMSO (3 mL) and purified by RP—ISCO chromatography (100 G ISCO Gold C18 Column, Water/MeCN as eluent, 0.1% TFA as modifier).

The desired product fractions were combined and lyophilized to afford the title compound (375 mg), which contained impurity. This product was used in the next step without further purification. LC/MS: MH⁺=454.5, Rt=0.78 min (2 min basic method).

### Synthesis of 17-((benzyloxy)carbonyl)-4,13,21-trioxo-3,7,10,24,27,30-hexaoxa-14,17,20-triazatritriacontan-33-oic acid

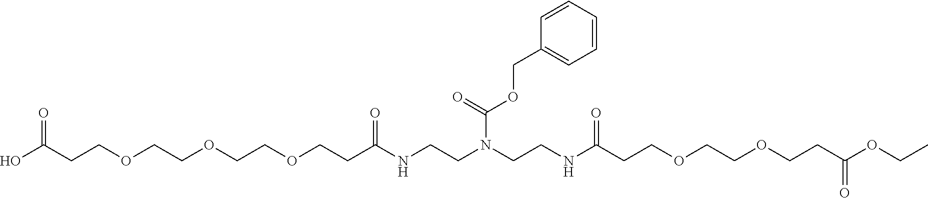 [SMILES: CCOC(=O)CCOCCOCCC(=O)NCCN(CCNC(=O)CCOCCOCCOCCC(=O)O)C(=O)OCc1ccccc1]

A mixture of ethyl 4-(2-aminoethyl)-3,8-dioxo-1-phenyl-2,11,14-trioxa-4,7-diazaheptadecan-17-oate (375 mg from the previous step), 3-(2-(2-(3-((2,5-dioxopyrrolidin-1-yl)oxy)-3-oxopropoxy)ethoxy)ethoxy)propanoic acid (210 mg, 0.605 mmol), and DIPEA (0.840 mL, 4.81 mmol) in DMF (4 mL) was stirred at RT for 5 min. The mixture was diluted with DMSO (5 mL) and loaded on RP-C18 ISCO column (gold, 150 g). The column was eluted with MeCN-water (0.1% TFA). Fractions containing the desired product were combined and concentrated to give the title compound as an oil (310 mg), containing a minor impurity. This will be used without further purification. LC/MS: MH⁺=686.6, Rt=0.80 min (2 min acidic method).

### Synthesis of 4,13,21-trioxo-3,7,10,24,27,30-hexaoxa-14,17,20-triazatritriacontan-33-oic acid

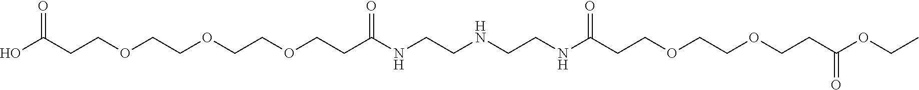 [SMILES: CCOC(=O)CCOCCOCCC(=O)NCCNCCNC(=O)CCOCCOCCOCCC(=O)O]

A mixture of 17-((benzyloxy)carbonyl)-4,13,21-trioxo-3,7,10,24,27,30-hexaoxa-14,17,20-triazatritriacontan-33-oic acid (305 mg, 0.445 mmol) and Pd—C(10% wet) (51 mg, 0.48 mmol) in EtOAc (5 mL) was hydrogenated under balloon pressure at RT for 1 h. The mixture was filtered through Celite with MeCN and water. The filtrate was concentrated to give the crude the title compound as an oil (231 mg), which was directly used in the next step without purification. LC/MS: MH⁺=552.5, Rt=0.50 min (2 min acidic method).

### Synthesis of 17-(3-(2-((tert-butoxycarbonyl)amino)ethoxy)propanoyl)-4,13,21-trioxo-3,7,10,24,27,30-hexaoxa-14,17,20-triazatritriacontan-33-oic acid

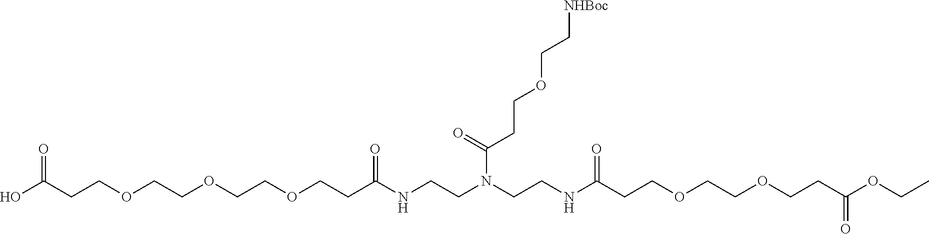 [SMILES: CCOC(=O)CCOCCOCCC(=O)NCCN(CCNC(=O)CCOCCOCCOCCC(=O)O)C(=O)CCOCCNC(=O)OC(C)(C)C]

A mixture of 3-(2-((tert-butoxycarbonyl)amino)ethoxy)propanoic acid (117 mg, 0.503 mmol), HATU (175 mg, 0.461 mmol), and DIPEA (0.30 mL, 1.7 mmol) in DMF (3 mL) was stirred at RT for 10 min. This solution was added to a DMF (1 mL) solution of 4,13,21-trioxo-3,7,10,24,27,30-hexaoxa-14,17,20-triazatritriacontan-33-oic acid (231 mg, 0.419 mmol). DIPEA (0.2 mL) was added. The mixture was stirred at RT for 10 min. The mixture was diluted with DMSO (5 mL) and loaded onto RP-C18 ISCO column (150 g, gold). The column was eluted with MeCN-water (0.1% TFA). Fractions containing the desired product were combined and lyophilized to give the product as an oil (163 mg). LC/MS: MH⁺=767.9, Rt=0.77 min (2 min acidic method).

### Synthesis of 2,2,5-trimethyl-4-oxo-3,8,11,14-tetraoxa-5-azaheptadecan-17-oic acid

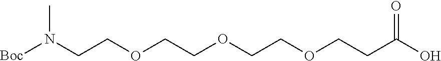 [SMILES: CN(CCOCCOCCOCCC(=O)O)C(=O)OC(C)(C)C]

To a solution of 3-(2-(2-(2-bromoethoxy)ethoxy)ethoxy)propanoic acid (1 g, 3.51 mmol) in THF (10 mL) was added MeNH2 in H₂O, 40% by weight, (26.7 grams, 344 mmol). After stirring overnight, the volatiles were removed in vacuo. The oil was dissolved in MeOH (10 mL) and THF (10 mL), DIEA (0.451 g, 3.51 mmol) was added followed by Boc2O (2.296 g, 10.5 mmol). After 2.5 hours, the volatiles were removed in vacuo, the residue was dissolved in DMSO and purified by ISCO RP-HPLC. Upon lyophilization, 2,2,5-trimethyl-4-oxo-3,8,11,14-tetraoxa-5-azaheptadecan-17-oic acid (1.10 g, 94% yield) was obtained. LC/MS: MH⁺=336.4, Rt=1.57 min (5 min acidic run).

### Synthesis of 2-cyanoethyl 5,8,11-trioxa-2-azatetradecan-14-oate

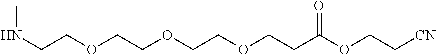 [SMILES: CNCCOCCOCCOCCC(=O)OCCC#N]

To a solution of 2,2,5-trimethyl-4-oxo-3,8,11,14-tetraoxa-5-azaheptadecan-17-oic acid (4.56 grams, 13.6 mmol), DMAP (332 mg, 2.72 mmol) and 3-hydroxypropanenitrile (0.996 mL, 14.28 mmol) in CH₂Cl₂ (100 mL) was added EDC (3.128 grams, 16.31 mmol). After stirring for 16 hours, the reactions was partitioned between EtOAc and H2O, mixed, separated, washed with 1M HCl, NaHCO₃(sat), NaCl(sat.), dried over MgSO₄, filtered and concentrated. The residue was treated with 25% TFA/CH₂Cl₂ (24 mL) for 1.5 hours at which time the volatiles were removed in vacuo and pumped on to yield 8.09 grams of 2-cyanoethyl 5,8,11-trioxa-2-azatetradecan-14-oate as trifluoroacetate salt. Based on weight, assumed 2.65 equivalents of TFA and used as such. LC/MS: MH⁺=289.2, Rt=0.48 min (5 min acidic method).

### Synthesis of allyl 5,8,11-trioxa-2-azatetradecan-14-oate

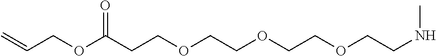 [SMILES: C=CCOC(=O)CCOCCOCCOCCNC]

To a solution of 2,2,5-trimethyl-4-oxo-3,8,11,14-tetraoxa-5-azaheptadecan-17-oic acid (3.026 grams, 9.022 mmol), DMAP (331 mg, 2.71 mmol) and allyl alcohol (0.689 mL, 9.92 mmol) in CH₂Cl₂ (50 mL) was added EDC (2.08 grams, 10.83 mmol). After stirring for 2 hours, the reactions was partitioned between EtOAc and H₂O, mixed, separated, washed with 1M HCl, NaHCO₃(sat), NaCl(sat.), dried over MgSO₄, filtered and concentrated. The residue was treated with 4M HCl in dioxane (24 mL, 96 mmol). After standing at rt for 14 hours, the volatiles were removed in vacuo and dissolved in MeCN/H₂O. Upon lyophilization, allyl 5,8,11-trioxa-2-azatetradecan-14-oate (2.55 grams, 82% yield) was obtained as HCl salt. LC/MS: MH⁺=276.3, Rt=0.72 min (5 min acidic method).

### Synthesis of 3-(tert-butoxycarbonyl)-6-methyl-5,18-dioxo-9,12,15,19-tetraoxa-3,6-diazadocos-21-enoic acid

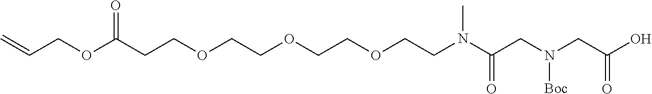 [SMILES: C=CCOC(=O)CCOCCOCCOCCN(C)C(=O)CN(CC(=O)O)C(=O)OC(C)(C)C]

To a solution of allyl 5,8,11-trioxa-2-azatetradecan-14-oate (1.944 grams, 9.03 mmol) and tert-butyl 3,5-dioxopiperidine-1-carboxylate HCl salt (2.817 grams, 9.03 mmol) in DMF (5 mL) was added DIEA (3.15 mL, 18.07 mmol). After stirring at rt for 2 hours, the solution was diluted with DMSO and purified by RP—ISCO. Upon lyophilization, 3-(tert-butoxycarbonyl)-6-methyl-5,18-dioxo-9,12,15,19-tetraoxa-3,6-diazadocos-21-enoic acid (2.62 grams, 59% yield) was obtained. LC/MS: MH⁺=491.5, Rt=1.85 min (5 min acidic method).

### Synthesis of 1-allyl 31-(2-cyanoethyl) 16-(tert-butoxycarbonyl)-13,19-dimethyl-14,18-dioxo-4,7,10,22,25,28-hexaoxa-13,16,19-triazahentriacontanedioate

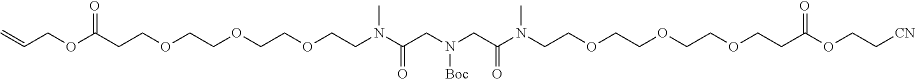 [SMILES: C=CCOC(=O)CCOCCOCCOCCN(C)C(=O)CN(CC(=O)N(C)CCOCCOCCOCCC(=O)OCCC#N)C(=O)OC(C)(C)C]

To a solution of 2-cyanoethyl 5,8,11-trioxa-2-azatetradecan-14-oate trifluoroacetate (2.65 equiv) (3.406 grams, 5.768 mmol) in CH₂Cl₂ (10 mL) was added DIEA (4.65 mL, 26.7 mmol). The solution was added to 3-(tert-butoxycarbonyl)-6-methyl-5,18-dioxo-9,12,15,19-tetraoxa-3,6-diazadocos-21-enoic acid (2.620 grams, 5.34 mmol) and HOAT (873 mg, 6.41 mmol) and EDC (1.229 grams, 6.41 mmol) was then added last. After stirring at rt for 5 hours, the volatiles were removed in vacuo, the residue was dissolved in DMSO and purified by RP—ISCO. After lyophilization, 1-allyl 31-(2-cyanoethyl) 16-(tert-butoxycarbonyl)-13,19-dimethyl-14,18-dioxo-4,7,10,22,25,28-hexaoxa-13,16,19-triazahentriacontanedioate (3.57 grams, 87% yield) was obtained. LC/MS: MH⁺=761.7, Rt=1.95 min (5 min acidic method).

### Synthesis of 1-allyl 31-(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate

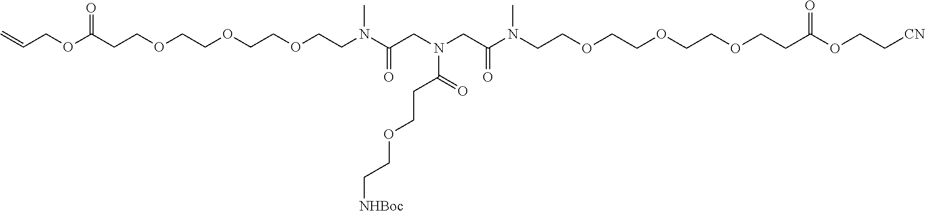 [SMILES: C=CCOC(=O)CCOCCOCCOCCN(C)C(=O)CN(CC(=O)N(C)CCOCCOCCOCCC(=O)OCCC#N)C(=O)CCOCCNC(=O)OC(C)(C)C]

1-allyl 31-(2-cyanoethyl) 16-(tert-butoxycarbonyl)-13,19-dimethyl-14,18-dioxo-4,7,10,22,25,28-hexaoxa-13,16,19-triazahentriacontanedioate (803 mg, 1.06 mmol) was treated with 4M HCl in dioxane (5 mL, 40 μmol) for 12 hours at which time the volatiles were removed in vacuo. The residue was dissolved in NMP (3 mL) and was neutralized by adding DIEA (0.5 mL, 2.87 mmol). 3-(2-((tert-butoxycarbonyl)amino)ethoxy)propanoic acid (320 mg, 1.37 mmol), HOAT (187 mg, 1.37 mmol) and EDC (263 mg, 1.37 mmol) were added and after stirring at rt for 2 hours the solution was diluted with DMSO and purified by RP—ISCO. After lyophilization, 1-allyl 31-(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (657 mg, 71%) was obtained. LC/MS: MH⁺=876.7, Rt=2.07 min (5 min acidic method).

### Synthesis of 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid

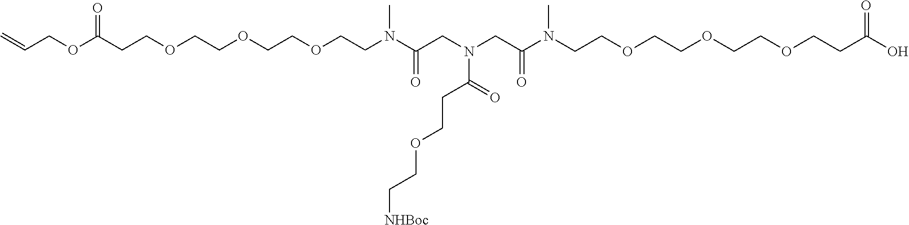 [SMILES: C=CCOC(=O)CCOCCOCCOCCN(C)C(=O)CN(CC(=O)N(C)CCOCCOCCOCCC(=O)O)C(=O)CCOCCNC(=O)OC(C)(C)C]

To a solution of 1-allyl 31-(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (375 mg, 428 μmol) in DMSO (1 mL) was added DBU (129 μL, 856 μmol). After standing for two hours, the solution was purified by ISCO C18 RP-HPLC with 0.05% formic acid modifier. After lyophilization, 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (232 mg, 66% yield) was obtained. LC/MS: MH⁺=823.7, Rt=1.86 min (5 min acidic method).

### Synthesis of tert-butyl 11-(2-(tert-butoxy)-2-oxoethyl)-1-(9H-fluoren-9-yl)-3,10-dioxo-2,7-dioxa-4,11-diazatridecan-13-oate

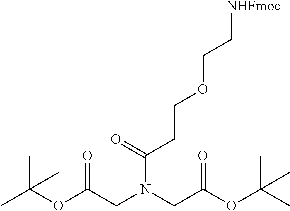

To a solution of di-tert-butyl 2,2′-azanediyldiacetate (250 mg, 1019 μmol) and HATU (407 mg, 1070 μmol) in DMF (2 mL) was added DIEA (356 μL, 2038 μmol). After stirring for 5 minutes, 3-(2-((((9H-fluoren-9-yl)methoxy)carbonyl)amino)ethoxy)propanoic acid (398 mg, 1121 μmol) was added. After stirring for an additional 30 minutes, the solution was diluted with DMSO and purified by ISCO RP-HPLC. Upon lyophilization, tert-butyl 11-(2-(tert-butoxy)-2-oxoethyl)-1-(9H-fluoren-9-yl)-3,10-dioxo-2,7-dioxa-4,11-diazatridecan-13-oate (509 mg, 86% yield) was obtained. LC/MS: MH⁺=583.4, Rt=3.06 min (5 min acidic method).

### Synthesis of 11-(carboxymethyl)-1-(9H-fluoren-9-yl)-3,10-dioxo-2,7-dioxa-4,11-diazatridecan-13-oic acid

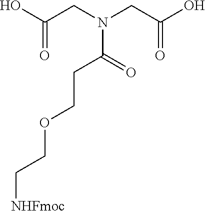

tert-butyl 11-(2-(tert-butoxy)-2-oxoethyl)-1-(9H-fluoren-9-yl)-3,10-dioxo-2,7-dioxa-4,11-diazatridecan-13-oate (509 mg, 874 μmol) was treated with 33% TFA/CH₂Cl₂ (3 mL) for 4 hours at which time the volatiles were removed in vacuo. The residue was dissolved in MeCN/H₂O and upon lyophilization 11-(carboxymethyl)-1-(9H-fluoren-9-yl)-3,10-dioxo-2,7-dioxa-4,11-diazatridecan-13-oic acid (400 mg, 97% yield) was obtained. LC/MS: MH⁺=471.0, Rt=1.87 min (5 min acidic method).

### Synthesis of di-tert-butyl 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate

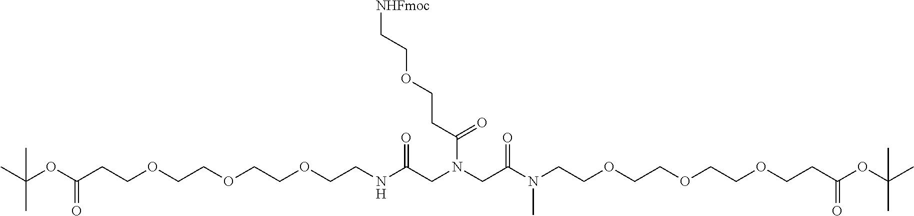

To a solution of 11-(carboxymethyl)-1-(9H-fluoren-9-yl)-3,10-dioxo-2,7-dioxa-4,11-diazatridecan-13-oic acid (40 mg, 85 μmol) and HATU (71 mg, 187 μmol) in DMF (2 mL) was added DIEA (74 μL, 74 μmol). After stirring for 5 minutes, tert-butyl 5,8,11-trioxa-2-azatetradecan-14-oate (74 mg, 255 μmol) was added. After stirring for an additional 30 minutes, the solution was diluted with DMSO and purified by ISCO RP-HPLC. Upon lyophilization, di-tert-butyl 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate (54 mg, 62% yield) was obtained. LC/MS: MH⁺=1017.8, Rt=3.01 min (5 min acidic method).

### Synthesis of 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioic acid

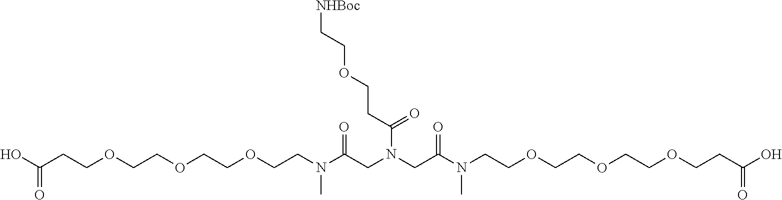 [SMILES: CN(CCOCCOCCOCCC(=O)O)C(=O)CN(CC(=O)N(C)CCOCCOCCOCCC(=O)O)C(=O)CCOCCNC(=O)OC(C)(C)C]

di-tert-butyl 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate (54 mg, 53 μmol) was treated with 33% TFA/CH₂Cl₂ (3 mL) for 4 hours at which time the volatiles were removed in vacuo. The residue was dissolved in MeCN/H₂O and upon lyophilization 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioic acid (47.4 mg, 99% yield) was obtained. HRMS: MH⁺=905.4400, Rt=2.04 min (5 min acidic method).

### Synthesis of bis(2-cyanoethyl) 16-(tert-butoxycarbonyl)-13,19-dimethyl-14,18-dioxo-4,7,10,22,25,28-hexaoxa-13,16,19-triazahentriacontanedioate

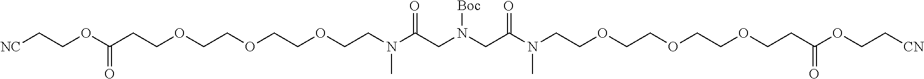 [SMILES: CN(CCOCCOCCOCCC(=O)OCCC#N)C(=O)CN(CC(=O)N(C)CCOCCOCCOCCC(=O)OCCC#N)C(=O)OC(C)(C)C]

To 2-cyanoethyl 5,8,11-trioxa-2-azatetradecan-14-oate HCl salt (3.19 grams, 8.83 mmol) and tert-butyl 2,6-dioxomorpholine-4-carboxylate (0.95 grams, 4.41 mmol) in 1:2 NMP/CH₂Cl₂ (15 mL) was added DIEA (2.31 mL, 13.2 mmol), HOAT (0.661 g, 4.86 mmol) and then EDC (0.931 g, 4.86 mmol). After stirring for 2 hours, the volatiles were removed in vacuo, DMSO was added and the solution was purified by RP-HPLC (with 0.05% formic acid modifier). Upon lyophilization, bis(2-cyanoethyl) 16-(tert-butoxycarbonyl)-13,19-dimethyl-14,18-dioxo-4,7,10,22,25,28-hexaoxa-13,16,19-triazahentriacontanedioate (2.10 g, 61% yield) was obtained. LC/MS: MH⁺=774.6, Rt=1.77 min (5 min acidic method).

### Synthesis of bis(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate

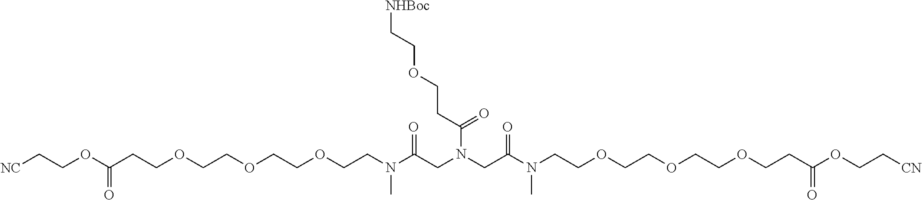 [SMILES: CN(CCOCCOCCOCCC(=O)OCCC#N)C(=O)CN(CC(=O)N(C)CCOCCOCCOCCC(=O)OCCC#N)C(=O)CCOCCNC(=O)OC(C)(C)C]

To bis(2-cyanoethyl) 16-(tert-butoxycarbonyl)-13,19-dimethyl-14,18-dioxo-4,7,10,22,25,28-hexaoxa-13,16,19-triazahentriacontanedioate (2.10 g, 2.71 mmol) was added 4 M HCl in dioxane (10 mL, 40 mmol). After standing for one hour, the volatiles were removed in vacuo. The residue was dissolved in CH₂Cl₂ (10 mL) and DIEA (1.65 mL, 9.5 mmol) was added as the solution stirred. To the neutralized solution was added HOAT (480 mg, 3.5 mmol), 3-(2-((tert-butoxycarbonyl)amino)ethoxy)propanoic acid (791 mg, 3.4 mmol) and EDC (676 mg, 3.5 mmol). After stirring for two hours, the volatiles were removed in vacuo, DMSO was added and the solution was purified by RP-HPLC (with 0.05% formic acid modifier). Upon lyophilization, bis(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (2.30 g, 95% yield) was obtained. LC/MS: MH⁺=889.7

### Synthesis of 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioic acid

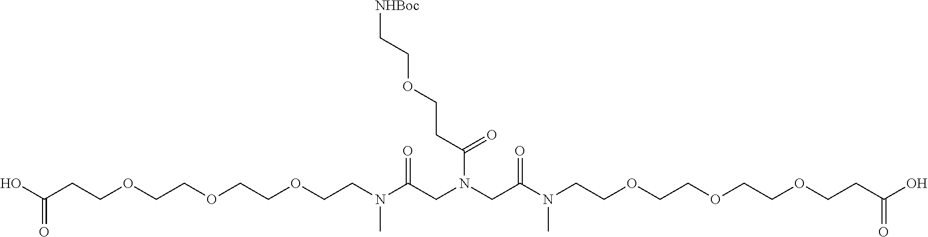 [SMILES: CN(CCOCCOCCOCCC(=O)O)C(=O)CN(CC(=O)N(C)CCOCCOCCOCCC(=O)O)C(=O)CCOCCNC(=O)OC(C)(C)C]

To bis(2-cyanoethyl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (2.3 g, 2.587 mmol) in DMSO (15 mL) was added DBU (1.56 mL, 10.35 mmol). After stirring for one hour, the solution was directly purified by RP-HPLC (with 0.05% formic acid modifier). Upon lyophilization, 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioic acid (0.771 g, 38% yield) was obtained. LC/MS: MH⁺=783.6, Rt=1.50 min (5 min acidic method).

### Synthesis of bis(2,5-dioxopyrrolidin-1-yl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate

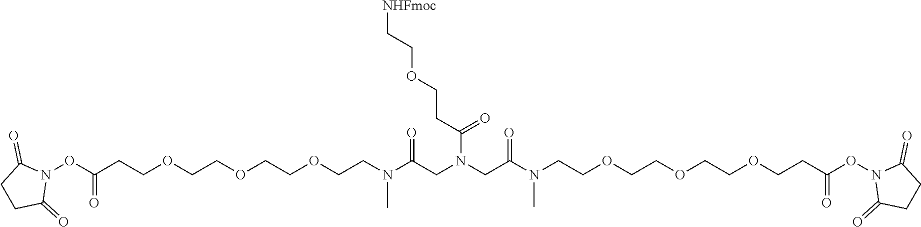

To 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioic acid (168 mg, 215 μmol) in NMP (2 mL) was added DIEA (94 μL, 537 μmol) and then TSTU (155 mg, 515 μmol). After stirring for one hour, the solution was diluted with DMSO and purified by RP-HPLC (with 0.05% formic acid modifier). Upon lyophilization, bis(2,5-dioxopyrrolidin-1-yl) 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioate (138 mg, 66% yield) was obtained. LC/MS: MH⁺=977.6, Rt=1.75 min (5 min acidic method).

### Synthesis of di-tert-butyl 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioate

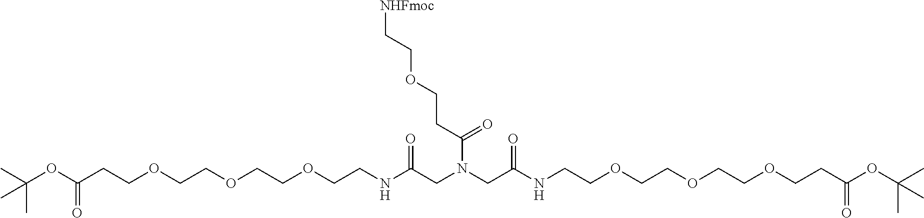

To a solution of 11-(carboxymethyl)-1-(9H-fluoren-9-yl)-3,10-dioxo-2,7-dioxa-4,11-diazatridecan-13-oic acid (100 mg, 213 μmol) and HATU (162 mg, 425 μmol) in DMF (2 mL) was added DIEA (111 μL, 638 μmol). After stirring for 5 minutes, tert-butyl 3-(2-(2-(2-aminoethoxy)ethoxy)ethoxy)propanoate (177 mg, 638 μmol) was added. After stirring for an additional 30 minutes, the solution was diluted with DMSO and purified by ISCO RP-HPLC. Upon lyophilization, di-tert-butyl 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioate (185 mg, 88% yield) was obtained. LC/MS: MH⁺=989.5300, Rt=2.77 min (5 min acidic method).

### Synthesis of 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioic acid

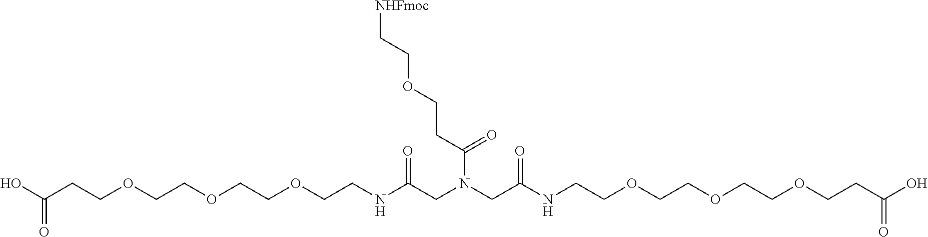

di-tert-butyl 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioate (185 mg, 187 μmol) was treated with 50% TFA/CH₂Cl₂ (44 mL) for 2 hours at which time the volatiles were removed in vacuo. The residue was dissolved in MeCN/H₂O and upon lyophilization 16-(3-(2-((((9H-fluoren-9-yl)methoxy)carbonyl)amino)ethoxy)propanoyl)-14,18-dioxo-4,7,10,22,25,28-hexaoxa-13,16,19-triazahentriacontanedioic acid (146 mg, 89% yield) was obtained. LC/MS: MH⁺=877.6, Rt=1.91 min (5 min acidic method).

### Synthesis of tert-butyl (11-(2-((tert-butoxycarbonyl)amino)ethyl)-1-(9H-fluoren-9-yl)-3,10-dioxo-2,7-dioxa-4,11-diazatridecan-13-yl)carbamate

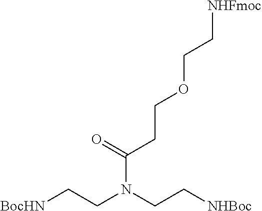

To a solution of 3-(2-((((9H-fluoren-9-yl)methoxy)carbonyl)amino)ethoxy)propanoic acid (322 mg, 906 μmol) and HATU (329 mg, 865 μmol) in DMF (2 mL) was added DIEA (288 μL, 1648 μmol). After stirring for 5 minutes, di-tert-butyl (azanediylbis(ethane-2,1-diyl))dicarbamate (250 mg, 824 μmol) was added. After stirring for an additional 30 minutes, the solution was diluted with DMSO and purified by ISCO RP-HPLC. Upon lyophilization, tert-butyl (11-(2-((tert-butoxycarbonyl)amino)ethyl)-1-(9H-fluoren-9-yl)-3,10-dioxo-2,7-dioxa-4,11-diazatridecan-13-yl)carbamate (528 mg, 85% yield) was obtained. LC/MS: MH⁺=641.4, Rt=2.82 min (5 min acidic method).

### Synthesis of (9H-fluoren-9-yl)methyl (2-(3-(bis(2-aminoethyl)amino)-3-oxopropoxy)ethyl)carbamate

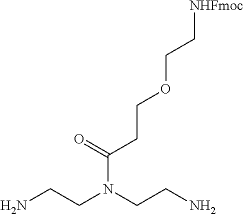

tert-butyl (11-(2-((tert-butoxycarbonyl)amino)ethyl)-1-(9H-fluoren-9-yl)-3,10-dioxo-2,7-dioxa-4,11-diazatridecan-13-yl)carbamate (450 mg, 702 μmol) was treated with 25% TFA/CH₂Cl₂ (3 mL) for 1 hours at which time the volatiles were removed in vacuo. The residue was dissolved in MeCN/H₂O and upon lyophilization (9H-fluoren-9-yl)methyl (2-(3-(bis(2-aminoethyl)amino)-3-oxopropoxy)ethyl)carbamate (480 mg) was obtained. LC/MS: MH⁺=441.1, Rt=0.94 min (5 min acidic method).

### Synthesis of tert-butyl 11-(2-aminoethyl)-1-(9H-fluoren-9-yl)-3,10,15-trioxo-2,7,18,21-tetraoxa-4,11,14-triazatetracosan-24-oate

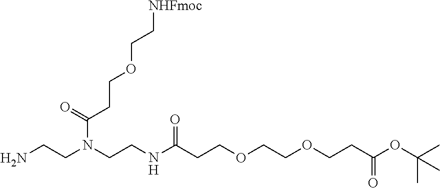

To a solution of 3-(2-(3-(tert-butoxy)-3-oxopropoxy)ethoxy)propanoic acid (30 mg, 114 μmol) and HATU (43.5 mg, 114 μmol) in DMF (2 mL) was added DIEA (120 μL, 686 μmol). After stirring for 5 minutes, (9H-fluoren-9-yl)methyl (2-(3-(bis(2-aminoethyl)amino)-3-oxopropoxy)ethyl)carbamate TFA salt (308 mg, 343 μmol) was added. After stirring for an additional 30 minutes, the solution was diluted with DMSO and purified by ISCO RP-HPLC. Upon lyophilization, tert-butyl 11-(2-aminoethyl)-1-(9H-fluoren-9-yl)-3,10,15-trioxo-2,7,18,21-tetraoxa-4,11,14-triazatetracosan-24-oate (65 mg, 62% yield) was obtained. LC/MS: MH⁺=685.5, Rt=2.05 min (5 min acidic method).

### Synthesis of di-tert-butyl 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioate

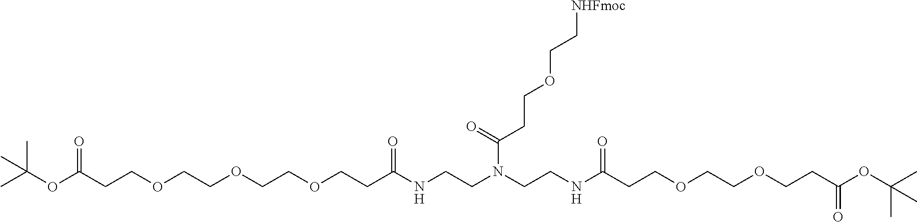

To a solution of 2,2-dimethyl-4-oxo-3,7,10,13-tetraoxahexadecan-16-oic acid (26 mg, 85 μmol) and HATU (32.2 mg, 85 μmol) in DMF (1 mL) was added DIEA (62 μL, 353 μmol). After stirring for 5 minutes, tert-butyl 11-(2-aminoethyl)-1-(9H-fluoren-9-yl)-3,10,15-trioxo-2,7,18,21-tetraoxa-4,11,14-triazatetracosan-24-oate TFA salt (65 mg, 71 μmol) was added. After stirring for an additional 30 minutes, the solution was diluted with DMSO and purified by ISCO RP-HPLC. Upon lyophilization, di-tert-butyl 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioate (46 mg, 67% yield) was obtained. LC/MS: MH⁺=973.8, Rt=2.84 min (5 min acidic method).

### Synthesis of 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioic acid

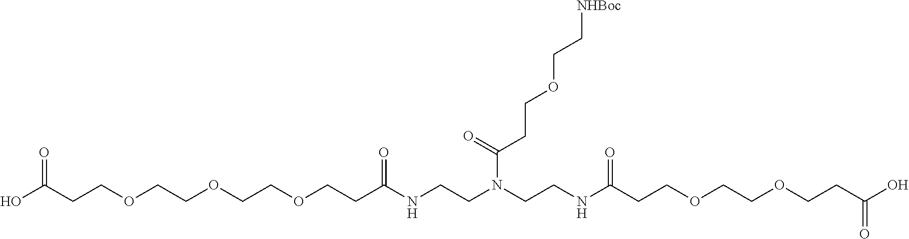 [SMILES: CC(C)(C)OC(=O)NCCOCCC(=O)N(CCNC(=O)CCOCCOCCOCCC(=O)O)CCNC(=O)CCOCCOCCC(=O)O]

di-tert-butyl 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioate (46.4 mg, 48 μmol) was treated with 50% TFA/CH₂Cl₂ (2 mL) for 2 hours at which time the volatiles were removed in vacuo. The residue was dissolved in MeCN/H₂O and upon lyophilization 17-(3-(2-((((9H-fluoren-9-yl)methoxy)carbonyl)amino)ethoxy)propanoyl)-13,21-dioxo-4,7,10,24,27-pentaoxa-14,17,20-triazatriacontanedioic acid (46 mg, 99% yield) was obtained. LC/MS: MH⁺=861.4, Rt=1.87 min (5 min acidic method).

### Synthesis of di-tert-butyl 10-(3-(2-((((9H-fluoren-9-yl)methoxy)carbonyl)amino)ethoxy)propanoyl)-8,12-dioxo-4,16-dioxa-7,10,13-triazanonadecanedioate

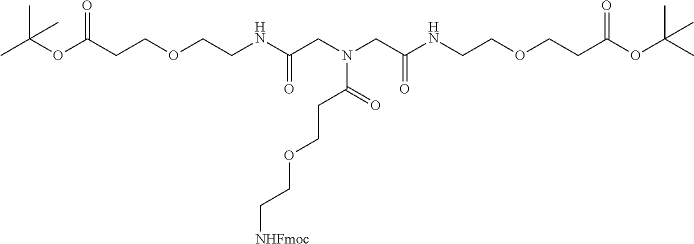

To a solution of 11-(carboxymethyl)-1-(9H-fluoren-9-yl)-3,10-dioxo-2,7-dioxa-4,11-diazatridecan-13-oic acid (50 mg, 106 μmol) and HATU (81 mg, 213 μmol) in DMF (2 mL) was added DIEA (93 μL, 531 μmol). After stirring for 45 minutes, tert-butyl 3-(2-aminoethoxy)propanoate (60.3 mg, 319 μmol) was added. After stirring for an additional 60 minutes, the solution was diluted with DMSO and purified by ISCO RP-HPLC. Upon lyophilization, di-tert-butyl 10-(3-(2-((((9H-fluoren-9-yl)methoxy)carbonyl)amino)ethoxy)propanoyl)-8,12-dioxo-4,16-dioxa-7,10,13-triazanonadecanedioate (44 mg, 51% yield) was obtained. LC/MS: MH⁺=813.7, Rt=2.95 min (5 min acidic method).

### Synthesis of 10-(3-(2-((((9H-fluoren-9-yl)methoxy)carbonyl)amino)ethoxy)propanoyl)-8,12-dioxo-4,16-dioxa-7,10,13-triazanonadecanedioic acid

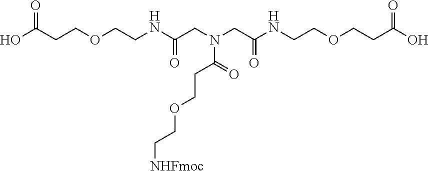

di-tert-butyl 10-(3-(2-((((9H-fluoren-9-yl)methoxy)carbonyl)amino)ethoxy)propanoyl)-8,12-dioxo-4,16-dioxa-7,10,13-triazanonadecanedioate (44 mg, 54 μmol) was treated with 33% TFA/CH₂Cl₂ (3 mL) for 1 hour at which time the volatiles were removed in vacuo. The residue was dissolved in MeCN/H₂O and upon lyophilization 10-(3-(2-((((9H-fluoren-9-yl)methoxy)carbonyl)amino)ethoxy)propanoyl)-8,12-dioxo-4,16-dioxa-7,10,13-triazanonadecanedioic acid (47 mg) was obtained. LC/MS: MH⁺=701.4, Rt=1.76 min (5 min acidic method).

### Synthesis of 1-(tert-butyl) 18-methyl 3-(((9H-fluoren-9-yl)methoxy)carbonyl)-5-oxo-9,12,15-trioxa-3,6-diazaoctadecanedioate

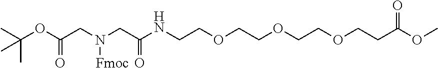

To a solution of N-(((9H-fluoren-9-yl)methoxy)carbonyl)-N-(2-(tert-butoxy)-2-oxoethyl)glycine (1 g, 2.43 mmol) and HATU (970 mg, 2.55 mmol) in DMF (12 mL) was added DIEA (1.7 mL, 9.7 mmol). After stirring for 5 minutes, methyl 3-(2-(2-(2-aminoethoxy)ethoxy)ethoxy)propanoate (899 mg, 2.67 mmol) was added. After stirring for an additional 30 minutes, the solution was poured into NH4Cl(sat.), was extracted with EtOAc and concentrated. Upon purification by SiO₂ chromatography and concentration, 1-(tert-butyl) 18-methyl 3-(((9H-fluoren-9-yl)methoxy)carbonyl)-5-oxo-9,12,15-trioxa-3,6-diazaoctadecanedioate (1.11 g, 73% yield) was obtained. LC/MS: MH⁺=629.5 and (M-tBu)+=573.4, Rt=2.70 min (5 min acidic method).

### Synthesis of 18-(((9H-fluoren-9-yl)methoxy)carbonyl)-3,16-dioxo-2,6,9,12-tetraoxa-15,18-diazaicosan-20-oic acid

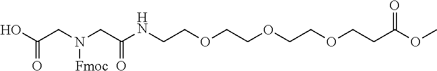

1-(tert-butyl) 18-methyl 3-(((9H-fluoren-9-yl)methoxy)carbonyl)-5-oxo-9,12,15-trioxa-3,6-diazaoctadecanedioate (1.11 g, 1.77 mol) was treated with 33% TFA/CH₂Cl₂ (6 mL) for 1 hour at which time dichloroethane (50 mL) was added and the volatiles were removed in vacuo. The material was used as is in the next step. LC/MS: MH⁺=573.4, Rt=0.96 min (2 min acidic method).

### Synthesis of 1-(tert-butyl) 31-methyl 16-(((9H-fluoren-9-yl)methoxy)carbonyl)-14,18-dioxo-4,7,10,22,25,28-hexaoxa-13,16,19-triazahentriacontanedioate

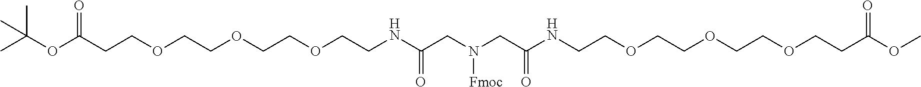

To a solution of 18-(((9H-fluoren-9-yl)methoxy)carbonyl)-3,16-dioxo-2,6,9,12-tetraoxa-15,18-diazaicosan-20-oic acid (1.33 g, 1.66 mmol) and HATU (695 mg, 1.83 mmol) in DMF (10 mL) was added DIEA (1.16 mL, 6.64 mmol). After stirring for 5 minutes, tert-butyl 3-(2-(2-(2-aminoethoxy)ethoxy)ethoxy)propanoate (599 mg, 2.16 mmol) was added. After stirring for an additional 30 minutes, the solution was poured into NH4Cl(sat.), was extracted with EtOAc and concentrated. Upon purification by SiO₂ chromatography and concentration, 1-(tert-butyl) 31-methyl 16-(((9H-fluoren-9-yl)methoxy)carbonyl)-14,18-dioxo-4,7,10,22,25,28-hexaoxa-13,16,19-triazahentriacontanedioate (1.19 g, 86% yield) was obtained. LC/MS: MH⁺=832.6, Rt=1.14 min (2 min acidic method).

### Synthesis of 18-(((9H-fluoren-9-yl)methoxy)carbonyl)-3,16,20-trioxo-2,6,9,12,24,27,30-heptaoxa-15,18,21-triazatritriacontan-33-oic acid

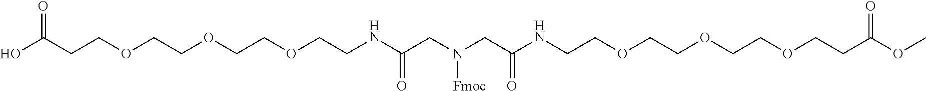

1-(tert-butyl) 31-methyl 16-(((9H-fluoren-9-yl)methoxy)carbonyl)-14,18-dioxo-4,7,10,22,25,28-hexaoxa-13,16,19-triazahentriacontanedioate (1.19 g, 1.46 mmol) was treated with 33% TFA/CH₂Cl₂ (4.5 mL) for one hour at which time the volatiles were removed in vacuo. The residue was dissolved in MeCN/H₂O and upon lyophilization 18-(((9H-fluoren-9-yl)methoxy)carbonyl)-3,16,20-trioxo-2,6,9,12,24,27,30-heptaoxa-15,18,21-triazatritriacontan-33-oic acid was obtained. LC/MS: MH⁺=776.6, Rt=0.94 min (2 min acidic method

### Synthesis of 3,16,20-trioxo-2,6,9,12,24,27,30-heptaoxa-15,18,21-triazatritriacontan-33-oic acid

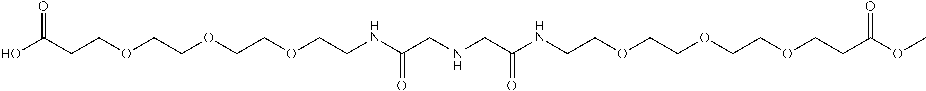 [SMILES: COC(=O)CCOCCOCCOCCNC(=O)CNCC(=O)NCCOCCOCCOCCC(=O)O]

A solution of 18-(((9H-fluoren-9-yl)methoxy)carbonyl)-3,16,20-trioxo-2,6,9,12,24,27,30-heptaoxa-15,18,21-triazatritriacontan-33-oic acid (1.47 g, 1.9 mmol) and 2.0 M dimethyl amine in THF (7 mL) was stirred at rt for one hour at which time the volatiles were removed in vacuo. The residue was dissolved in MeCN and neutralized with TFA (150 μL). The volatiles were removed in vacuo, the residue was dissolved in DMSO and purified by ISCO RP-HPLC. Upon lyophilization, 3,16,20-trioxo-2,6,9,12,24,27,30-heptaoxa-15,18,21-triazatritriacontan-33-oic acid (744 mg, 59% yield) was obtained. LC/MS: MH⁺=554.5, Rt=0.72 min (5 min acidic method).

### Synthesis of 18-(3-(2-((tert-butoxycarbonyl)amino)ethoxy)propanoyl)-3,16,20-trioxo-2,6,9,12,24,27,30-heptaoxa-15,18,21-triazatritriacontan-33-oic acid

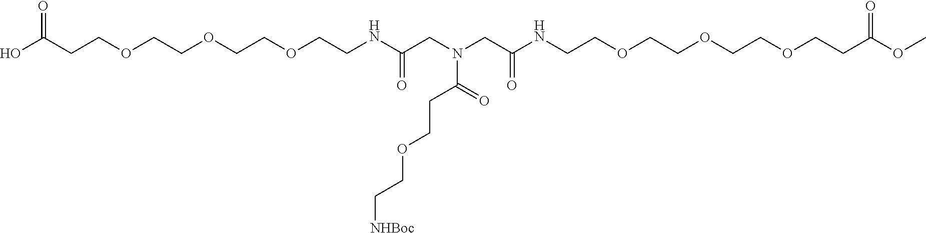 [SMILES: COC(=O)CCOCCOCCOCCNC(=O)CN(CC(=O)NCCOCCOCCOCCC(=O)O)C(=O)CCOCCNC(=O)OC(C)(C)C]

To a solution of 3-(2-((tert-butoxycarbonyl)amino)ethoxy)propanoic acid (470 mg, 2.02 mmol) and HATU (613 mg, 1.61 mmol) in DMF (6 mL) was added DIEA (704 μL, 4.03 mmol). After stirring for 5 minutes, 3,16,20-trioxo-2,6,9,12,24,27,30-heptaoxa-15,18,21-triazatritriacontan-33-oic acid (744 mg, 1.34 mmol) was added. After stirring for an additional 30 minutes, the solution was diluted with DMSO and purified by ISCO RP-HPLC. Upon lyophilization, 18-(3-(2-((tert-butoxycarbonyl)amino)ethoxy)propanoyl)-3,16,20-trioxo-2,6,9,12,24,27,30-heptaoxa-15,18,21-triazatritriacontan-33-oic acid (790 mg, 76% yield) was obtained. LC/MS: MH⁺=769.8, Rt=1.54 min (5 min acidic method).

### Synthesis of (9H-fluoren-9-yl)methyl (3-(bis(2-hydroxyethyl)amino)-3-oxopropyl)carbamate

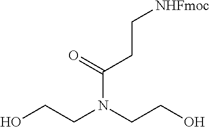

To a stirred solution of 3-((((9H-fluoren-9-yl)methoxy)carbonyl)amino)propanoic acid (2.96 g, 9.51 mmol, 1.00 equiv.) in dichloromethane (50 mL) was added 3-(((ethylimino)methylene)amino)-N,N-dimethylpropan-1-amine hydrochloride (2.006 g, 10.46 mmol, 1.10 equiv.). After stirring for 10 minutes at ambient temperature, diethanolamine (1.00 g, 9.51 mmol, 1.00 equiv.) was added and the resulting mixture was stirred at ambient temperature for 2 hours. The reaction mixture was concentrated under reduced pressure and the resulting residue was purified by chromatography on silica gel (0-20% methanol/dichloromethane) to afford (9H-fluoren-9-yl)methyl (3-(bis(2-hydroxyethyl)amino)-3-oxopropyl)carbamate (2.25 g, 5.65 mmol, 59% yield). LC/MS: MH⁺=399.3, Rt=0.82 min (2 min acidic run).

### Synthesis of (9H-fluoren-9-yl)methyl (3-(bis(2-(((4-nitrophenoxy)carbonyl)oxy)ethyl)amino)-3-oxopropylcarbamate

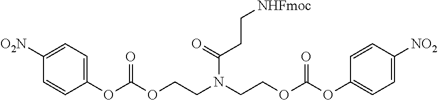

To a stirred solution of (9H-fluoren-9-yl)methyl (3-(bis(2-hydroxyethyl)amino)-3-oxopropyl)carbamate (2.24 g, 5.62 mmol, 1.00 equiv.) and bis(4-nitrophenyl) carbonate (3.76 g, 12.37 mmol, 2.20 equiv.) in dichloromethane (50 mL) was added N-ethyldiisopropylamine (1.60 g, 12.37 mmol, 2.20 equiv.). The resulting mixture was stirred at ambient temperature for 2 hours.

The reaction mixture was concentrated under reduced pressure at or below ambient temperature, and the resulting residue was purified by chromatography on silica gel (0-100% ethyl acetate/heptanes) to afford (9H-fluoren-9-yl)methyl (3-(bis(2-(((4-nitrophenoxy)carbonyl)oxy)ethyl)amino)-3-oxopropyl)carbamate (2.00 g, 2.74 mmol, 48% yield). LC/MS: M+Na⁺=751.3, Rt=1.20 min (2 min acidic run). ¹H NMR (400 MHz, Chloroform-d) δ 8.27-8.22 (m, 2H), 8.22-8.18 (m, 2H), 7.74 (d, J=7.4 Hz, 2H), 7.53 (d, J=7.4 Hz, 2H), 7.42-7.33 (m, 4H), 7.32-7.27 (m, 4H), 5.51 (t, J=6.3 Hz, 1H), 4.46 (dt, J=18.3, 5.3 Hz, 4H), 4.30 (d, J=7.2 Hz, 2H), 3.80 (dt, J=10.2, 5.4 Hz, 4H), 3.56 (d, J=5.8 Hz, 2H), 2.71 (t, J=5.5 Hz, 2H).

### Synthesis of tert-butyl (1-hydroxy-9-(2-(2-(2-hydroxyethoxy)ethoxy)ethyl)-10-oxo-3,6,13-trioxa-9-azapentadecan-15-yl)carbamate

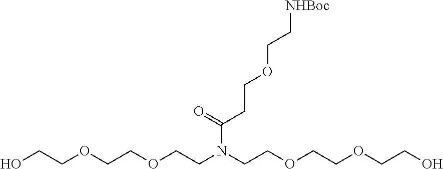 [SMILES: CC(C)(C)OC(=O)NCCOCCC(=O)N(CCOCCOCCO)CCOCCOCCO]

To a stirred solution of 3,6,12,15-tetraoxa-9-azaheptadecane-1,17-diol (116 mg, 0.412 mmol, 1.00 equiv.), 3-(2-((tert-butoxycarbonyl)amino)ethoxy)propanoic acid (101 mg, 0.433 mmol, 1.05 equiv.), and HATU (165 mg, 0.433 mmol, 1.05 equiv.) in DMF (1 mL) was added N-ethyldiisopropylamine (160 mg, 1.23 mmol, 3.00 equiv.). The resulting mixture was stirred at ambient temperature for 1 hour, then diluted with DMSO (2 mL) and purified by RP—ISCO. Upon lyophilization, tert-butyl (1-hydroxy-9-(2-(2-(2-hydroxyethoxy)ethoxy)ethyl)-10-oxo-3,6,13-trioxa-9-azapentadecan-15-yl)carbamate (118 mg, 0.238 mmol, 57% yield) was obtained. LC/MS: MH⁺=497.4, Rt=0.68 min (2 min acidic run).

### Synthesis of tert-butyl (6-oxo-7-(2-(2-(2-(prop-2-yn-1-yloxy)ethoxy)ethoxy)ethyl)-3,10,13,16-tetraoxa-7-azanonadec-18-yn-1-yl)carbamate

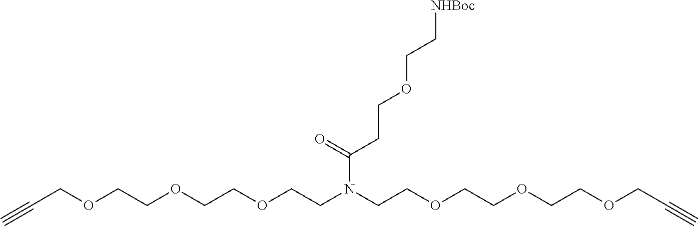 [SMILES: C#CCOCCOCCOCCN(CCOCCOCCOCC#C)C(=O)CCOCCNC(=O)OC(C)(C)C]

To a stirred solution of tert-butyl (1-hydroxy-9-(2-(2-(2-hydroxyethoxy)ethoxy)ethyl)-10-oxo-3,6,13-trioxa-9-azapentadecan-15-yl)carbamate (118 mg, 0.238 mmol, 1.00 equiv.) in dry THF (2 mL) at 0° C. was added sodium hydride (60% dispersion in oil, 20.0 mg, 0.499 mmol, 2.1 equiv.). After stirring at 0° C. for 30 minutes, propargyl bromide (62.2 mg, 0.523 mmol, 2.2 equiv.) was added and the resulting mixture was allowed to warm to ambient temperature with stirring over 20 hours. After concentration under reduced pressure, the resulting residue was purified by chromatography on silica gel (0-20% isopropanol/dichloromethane) to afford tert-butyl (6-oxo-7-(2-(2-(2-(prop-2-yn-1-yloxy)ethoxy)ethoxy)ethyl)-3,10,13,16-tetraoxa-7-azanonadec-18-yn-1-yl)carbamate (12.6 mg, 0.022 mmol, 9.3% yield). LC/MS: MH⁺=573.4, Rt=0.95 min (2 min acidic run). 1H NMR (400 MHz, Chloroform-d) δ 4.19 (d, J=2.3 Hz, 4H), 3.74 (t, J=6.4 Hz, 2H), 3.71-3.54 (m, 24H), 3.50 (t, J=5.1 Hz, 2H), 3.28 (d, J=5.7 Hz, 2H), 2.67 (t, J=6.4 Hz, 2H), 2.44 (t, J=2.4 Hz, 2H), 1.44 (d, J=2.9 Hz, 9H).

### Synthesis of Tert-butyl (6-oxo-7-(2-(2-(prop-2-yn-1-yloxy)ethoxy)ethyl)-3,10,13-trioxa-7-azahexadec-15-yn-1-yl)carbamate

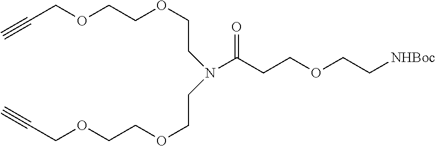 [SMILES: C#CCOCCOCCN(CCOCCOCC#C)C(=O)CCOCCNC(=O)OC(C)(C)C]

To the solution of NH-bis(PEG2-propargyl) (53 mg, 0.197 mmol), t-Boc-N-amido-PEG1-acid (48.2 mg, 0.207 mmol) in DMF (dry, 1 ml) was added HATU (79 mg, 0.207 mmol) and then DIPEA (103 μl, 0.590 mmol). The reaction mixture was stirred at RT for 1 h. The crude was purified by C-18 Column (50 g cartridge, eluted with MeCN/Water with 0.1% Formic Acid, 0-100% over 16 CV) to obtain Tert-butyl (6-oxo-7-(2-(2-(prop-2-yn-1-yloxy)ethoxy)ethyl)-3,10,13-trioxa-7-azahexadec-15-yn-1-yl)carbamate (81 mg, 85% yield). LC/MS: MH⁺=485.4, Rt=0.94 min (3 min acidic run).

### Synthesis of 3-(2-aminoethoxy)-N,N-bis(2-(2-(prop-2-yn-1-yloxy)ethoxy)ethyl)propanamide

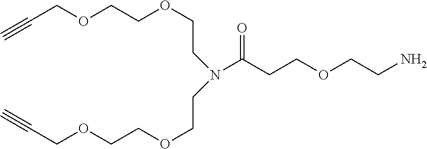 [SMILES: C#CCOCCOCCN(CCOCCOCC#C)C(=O)CCOCCN]

At 0° C. in an ice-water bath, to tert-butyl (6-oxo-7-(2-(2-(prop-2-yn-1-yloxy)ethoxy)ethyl)-3,10,13-trioxa-7-azahexadec-15-yn-1-yl)carbamate (81 mg, 0.167 mmol) was added TFA (25% in DCM) (2576 μl, 8.36 mmol). Then the reaction mixture was raised to RT and stirred for 90 min.

The mixture was concentrated under high vacuum at RT bath. Then the crude was separated with C-18 column (50 gram cartridge, MeCN/Water with 0.1% Formic acid 0-100% over 16 CV) to obtain 3-(2-aminoethoxy)-N,N-bis(2-(2-(prop-2-yn-1-yloxy)ethoxy)ethyl)propanamide (60 mg, 83% yield). LC/MS: MH⁺=385.8, Rt=0.56 min (3 min acidic run).

### Synthesis of Tert-butyl (S)-(17-oxo-4,7,10,13-tetraoxa-16-azahenicosa-1,20-diyn-18-yl)carbamate

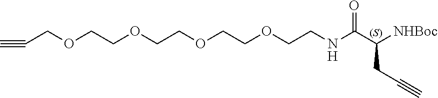

To a mixture of N-Boc-2-Propargyl-L-Glycine (100 mg, 0.469 mmol), Propargyl-PEG4-Amine (108 mg, 0.469 mmol), and TSTU (141 mg, 0.469 mmol) in DMF (2 ml) was added DIPEA (246 μl, 1.407 mmol). The reaction mixture was stirred at RT for 1 h. The crude mixture by C-18 column (100 g cartridge, 0-100% MeCN/Water with 0.05% TFA 0-100% over 16 CV) to obtain Tert-butyl (S)-(17-oxo-4,7,10,13-tetraoxa-16-azahenicosa-1,20-diyn-18-yl)carbamate (101 mg, 51% yield). LC/MS: MH⁺=427.5, Rt=0.83 min (3 min acidic run).

### Synthesis of (S)-2-amino-N-(3,6,9,12-tetraoxapentadec-14-yn-1-yl)pent-4-ynamide

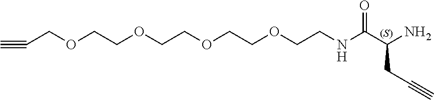

To tert-butyl (S)-(17-oxo-4,7,10,13-tetraoxa-16-azahenicosa-1,20-diyn-18-yl)carbamate (101 mg, 0.237 mmol) was added TFA (25% in DCM) (1460 μl, 4.74 mmol) at 0° C., then raise to RT. The mixture was stirred at rt for 1 h. The mixture was concentrated under high vacuum and then dried under high vacuum overnight to obtain (S)-2-amino-N-(3,6,9,12-tetraoxapentadec-14-yn-1-yl)pent-4-ynamide (TFA salt, 104 mg, 100% yield). LC/MS: MH⁺=327.4, Rt=0.49 min (3 min acidic run).

### Synthesis of tert-butyl (6-oxo-7-(2-(2-(prop-2-yn-1-yloxy)ethoxy)ethyl)-3,10,13-trioxa-7-azahexadec-15-yn-1-yl)carbamate

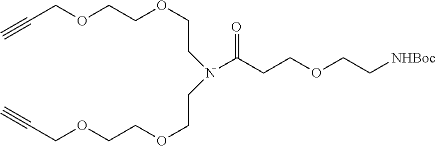 [SMILES: C#CCOCCOCCN(CCOCCOCC#C)C(=O)CCOCCNC(=O)OC(C)(C)C]

To the solution of bis(2-(2-(prop-2-yn-1-yloxy)ethoxy)ethyl)amine (53 mg, 0.197 mmol), 3-(2-((tert-butoxycarbonyl)amino)ethoxy)propanoic acid (48.2 mg, 0.207 mmol) in DMF (1 ml) was added HATU (79 mg, 0.207 mmol) and then DIPEA (103 μl, 0.590 mmol). The reaction mixture was stirred at RT for 1 h. The crude product was purified by RP-C18 ISCO chromatography (elution with MeCN-water, 0.1% formic acid) to give the title product as an oil (81 mg, 85% yield). LC/MS: MH⁺=485.5, Rt=0.92 min (2 min basic method).

### Synthesis of 3-(2-aminoethoxy)-N,N-bis(2-(2-(prop-2-yn-1-yloxy)ethoxy)ethyl)propanamide

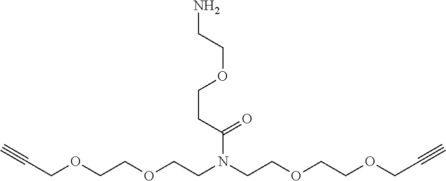 [SMILES: C#CCOCCOCCN(CCOCCOCC#C)C(=O)CCOCCN]

A mixture of tert-butyl (6-oxo-7-(2-(2-(prop-2-yn-1-yloxy)ethoxy)ethyl)-3,10,13-trioxa-7-azahexadec-15-yn-1-yl)carbamate (408 mg, 0.842 mmol) and TFA (1.62 mL, 21.1 mmol) in DCM (4.8 mL) was stirred at RT for 30 min and concentrated. The crude product was purified by RP-C18 ISCO chromatography (50 g, gold column)(elution with MeCN-water, 0.1% TFA). Fractions containing the desired product were combined and lyophilized to give the product (TFA salt) as a yellow oil (148 mg, 35% yield). LC/MS: MH⁺=385.3, Rt=0.55 min (2 min acidic method).

### Synthesis of 4,7,13,16,19-pentaoxa-10-azadocos-1-yn-22-oic acid

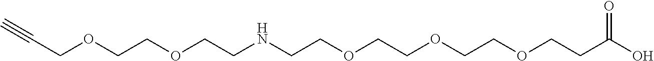 [SMILES: C#CCOCCOCCNCCOCCOCCOCCC(=O)O]

To tert-butyl 10-(tert-butoxycarbonyl)-4,7,13,16,19-pentaoxa-10-azadocos-1-yn-22-oate (250 mg, 0.496 mmol) and triethylsilane (238 μl, 1.489 mmol) in DCM (11.5 ml) at 0° C. was added the TFA (3824 μl, 49.6 mmol) dropwise (TFA/DCM 25% v/v). The mixture was stirred at 0° C. for 15 min, then raised to RT and stirred for 30 min. The crude mixture was concentrated on rotavap under high vacuum, then dry over high vacuum for two days to obtain 4,7,13,16,19-pentaoxa-10-azadocos-1-yn-22-oic acid (231 mg, as crude to use for next reaction without further purification). LC/MS: MH⁺=348.3, Rt=0.47 min (3 min acidic run).

### Synthesis of 2,2-dimethyl-4,11-dioxo-12-(2-(2-(prop-2-yn-1-yloxy)ethoxy)ethyl)-3,8,15,18,21-pentaoxa-5,12-diazatetracosan-24-oic acid

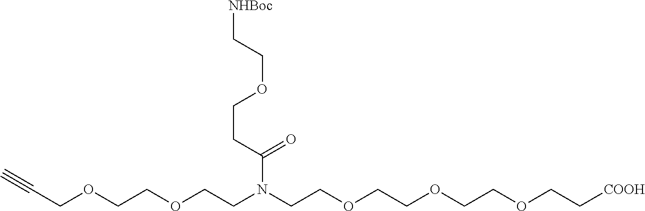 [SMILES: C#CCOCCOCCN(CCOCCOCCOCCC(=O)O)C(=O)CCOCCNC(=O)OC(C)(C)C]

To the solution t-Boc-N-amido-PEG1-acid (139 mg, 0.595 mmol) in DMF (dry, 2 ml) was added HATU (226 mg, 0.595 mmol) and then DIPEA (433 μl, 2.480 mmol). The reaction mixture was stirred at RT for 30 min. Then the solution of 4,7,13,16,19-pentaoxa-10-azadocos-1-yn-22-oic acid (229 mg, 0.496 mmol) in DMF (0.5 ml) was added to the above reaction mixture. The resulting mixture was stirred at RT for 30 min. The crude was separated by C-18 Column (100 g cartridge, eluted with MeCN/Water with 0.05% TFA, 0-100% over 16 CV) to obtain 2,2-dimethyl-4,11-dioxo-12-(2-(2-(prop-2-yn-1-yloxy)ethoxy)ethyl)-3,8,15,18,21-pentaoxa-5,12-diazatetracosan-24-oic acid (220 mg, 79% yield). LC/MS: MH⁺=563.4, Rt=0.83 min (3 min acidic run).

### Synthesis of allyl (2S,3S,4S,5R,6S)-3,4,5-tris(((allyloxy)carbonyl)oxy)-6-(2-amino-4-(((tert-butyldimethylsilyl)oxy)methyl)phenoxy)tetrahydro-2H-pyran-2-carboxylate

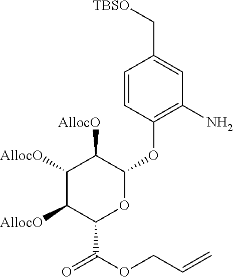

Zn powder (48.7 g, 745 mmol) was added portion wise to a mixture of allyl (2S,3S,4S,5R,6S)-3,4,5-tris(((allyloxy)carbonyl)oxy)-6-(4-(((tert-butyldimethylsilyl)oxy)methyl)-2-nitrophenoxy)tetrahydro-2H-pyran-2-carboxylate (Journal of Natural Products 2015, 78, 510) (56 g, 74.5 mmol), 1,4-dioxane (1.7 mL), H₂O (280 mL), and AcOH (42.6 mL, 745 mmol). The mixture was stirred at 25° C. for 16 h. The mixture was filtered through Celite and washed with 1,4-dioxane (500 mL) and DCM (500 mL). The resulting filtrate was neutralized with saturated NaHCO₃ and extracted with DCM. The combined organic extract were washed with saturated NaHCO₃ and brine. The organic layer was dried and concentrated in vacuo. The residue was purified by NP-column chromatography to give the title product as yellow oil (35.8 g, 66.7% yield). LC/MS: MH⁺=722.2.

### Synthesis of allyl (2S,3S,4S,5R,6S)-3,4,5-tris(((allyloxy)carbonyl)oxy)-6-(2-(3-((tert-butoxycarbonyl)amino)propanamido)-4-(((tert-butyldimethylsilyl)oxy)methyl)phenoxy)tetrahydro-2H-pyran-2-carboxylate

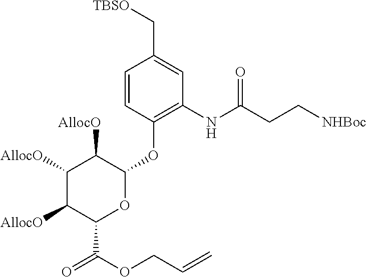

A mixture of allyl (2S,3S,4S,5R,6S)-3,4,5-tris(((allyloxy)carbonyl)oxy)-6-(2-amino-4-(((tert-butyldimethylsilyl)oxy)methyl)phenoxy)tetrahydro-2H-pyran-2-carboxylate (3.00 g, 4.16 mmol), 3-((tert-butoxycarbonyl)amino)propanoic acid (0.95 g, 5.02 mmol), HBTU (1.90 g, 5.01 mmol), and DIPEA (2.22 g, 17.2 mmol) in DMF (15 mL) was stirred at RT for 30 min, and partitioned between EtOAc and brine. The combined organic extract was dried over Na2SO4, concentrated, and chromatographed (NP—ISCO column) to give the title compound (1.66 g, 45% yield). LC/MS: MH⁺=893.7, Rt=1.49 min (2 min acidic method).

### Synthesis of allyl (2S,38,4,5R,6S)-3,4,5-tris(((allyloxy)oxy)-6-(2-(3-aminopropanamido)-4-(hydroxymethyl)phenoxy)tetrahydro-2H-pyran-2-carboxylate

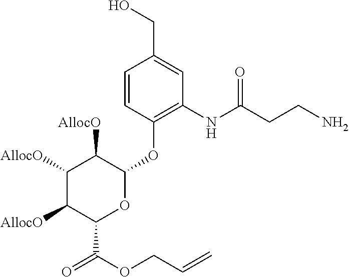

A mixture of allyl (2S,3S,4S,5R,6S)-3,4,5-tris(((allyloxy)carbonyl)oxy)-6-(2-(3-((tert-butoxycarbonyl)amino)propanamido)-4-(((tert-butyldimethylsilyl)oxy)methyl)phenoxy)tetrahydro-2H-pyran-2-carboxylate (1.66 g, 1.86 mmol) and pTsOH (0.036 g, 0.190 mmol) in MeOH (10 mL) was stirred at RT for 10 min. The mixture was concentrated to give the crude O-desilylated product as an oil (LC/MS MH⁺779.6, Rt=1.13 min (2 min acidic method)) which was treated with TFA (6.0 mL, 78 mmol) in DCM (4 mL) at RT for 10 min. The mixture was concentrated to give the crude title product which was used in the next step without purification. LC/MS: MH⁺=679.6, Rt=0.88 min (2 min acidic method).

### Synthesis of allyl (2S,3S,4S,5R,6S)-6-(2-(3-((S)-6-((((9H-fluoren-9-yl)methoxy)carbonyl)amino)-2-((tert-butoxycarbonyl)amino)hexanamido)propanamido)-4-(hydroxymethyl)phenoxy)-3,4,5-tris(((allyloxy)carbonyl)oxy)tetrahydro-2H-pyran-2-carboxylate

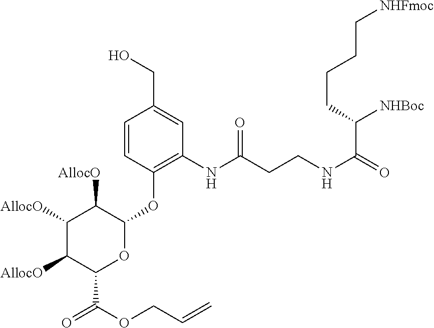

A mixture of allyl (2S,3S,4S,5R,6S)-3,4,5-tris(((allyloxy)carbonyl)oxy)-6-(2-(3-aminopropanamido)-4-(hydroxymethyl)phenoxy)tetrahydro-2H-pyran-2-carboxylate (crude obtained in the previous step), N6-(((9H-fluoren-9-yl)methoxy)carbonyl)-N2-(tert-butoxycarbonyl)-L-lysine (1.2 g, 2.6 mmol), HBTU (1.00 g, 2.64 mmol), and DIPEA (2.00 mL, 11.5 mmol) in DMF (10 mL) was stirred at RT for 10 min. The mixture was partitioned between EtOAc and sequentially 1M HCl and aqueous K₂CO₃. The combined organic extract was dried over Na₂SO₄ and concentrated. The crude product was purified by chromatography (NP—ISCO column, 80 g gold) to give the title product as a white solid (1.11 g, 53% yield over the last three steps). Synthesis of allyl (2S,3S,4S,5R,6S)-6-(2-(3-((S)-6-((((9H-fluoren-9-yl)methoxy)carbonyl)amino)-2-((tert-butoxycarbonyl)amino)hexanamido)propanamido)-4-(chloromethyl)phenoxy)-3,4,5-tris(((allyloxy)carbonyl)oxy)tetrahydro-2H-pyran-2-carboxylate

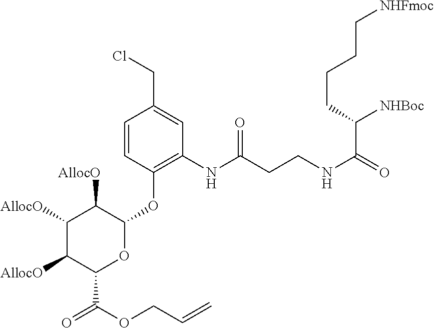

A mixture of allyl (2S,3S,4S,5R,6S)-6-(2-(3-((S)-6-((((9H-fluoren-9-yl)methoxy)carbonyl)amino)-2-((tert-butoxycarbonyl)amino)hexanamido)propanamido)-4-(hydroxymethyl)phenoxy)-3,4,5-tris(((allyloxy)carbonyl)oxy)tetrahydro-2H-pyran-2-carboxylate (1.11 g, 0.983 mmol), thionyl chloride (0.180 mL, 2.466 mmol), and sodium bicarbonate (0.495 g, 5.90 mmol) in THF (10 mL) was stirred at RT for 10 min. The mixture was partitioned between EtOAc and water. The combined organic extract was dried over Na₂SO₄, concentrated and chromatographed by NP—ISCO column. Fractions containing the desired product were combined and concentrated to give the title product as a white solid (0.95 g, 84% yield). LC/MS: MH⁺=1147.9, Rt=1.39 min (2 min acidic method).

### Synthesis of (S)-9-amino-1-(9H-fluoren-9-yl)-3,10-dioxo-2,14,17-trioxa-4,11-diazaicosan-20-oic acid

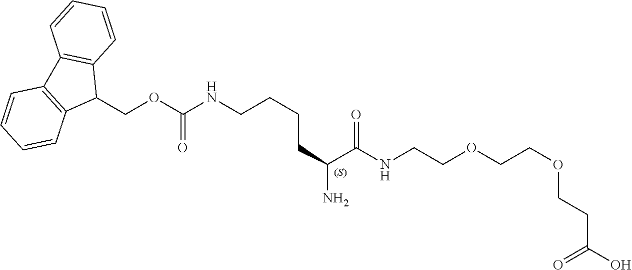

To a clear solution of N6-(((9H-fluoren-9-yl)methoxy)carbonyl)-N2-(tert-butoxycarbonyl)-L-lysine (633 mg, 1.350 mmol) and tert-butyl 3-(2-(2-aminoethoxy)ethoxy)propanoate (300 mg, 1.29 mmol) in Acetonitrile (9 ml) at 0° C. was added DMTMM (340 mg, 1.414 mmol). To the slurry was added DIPEA (0.449 ml, 2.57 mmol). The mixture was stirred at 0° C. for 5 min and then rt for 1.5 h. The solvent was removed by rotary evaporation and under high vac. To the resulting oily material was added CH₂Cl₂ (9 ml), triethylsilane (0.205 ml, 1.286 mmol) and TFA (4.95 ml, 64.3 mmol) at 0° C. The mixture was stirred at rt for 1.5 h. The solvent was removed via rotary evaporation and then under high vac. □□ The resulting oil was dissolved in DMSO/water and was purified by RP-HPLC (C18, ACN/water with 0.1% TFA) to afford, after lyophilization, a white powder as (S)-9-amino-1-(9H-fluoren-9-yl)-3,10-dioxo-2,14,17-trioxa-4,11-diazaicosan-20-oic acid as a TFA salt (754 mg, 91% yield). LC/MS: MH⁺=528.3, rt=0.79 min (2 min acidic method). ¹H NMR (400 MHz, DMSO-d6) δ 8.48 (t, J=5.6 Hz, 1H), 8.24-8.00 (m, 3H), 7.90 (d, J=7.5 Hz, 2H), 7.68 (d, J=7.5 Hz, 2H), 7.47-7.37 (m, 2H), 7.37-7.29 (m, 2H), 7.29-7.19 (m, 1H), 4.31 (d, J=6.8 Hz, 2H), 4.21 (t, J=6.8 Hz, 1H), 3.94-3.65 (m, 1H), 3.59 (dd, J=7.5, 5.2 Hz, 2H), 3.45 (dd, J=13.3, 7.7 Hz, 3H), 3.39-3.14 (m, 2H), 3.01-2.89 (m, 2H), 2.44 (t, J=6.3 Hz, 2H), 1.81-1.59 (m, 2H), 1.46-1.15 (m, 4H).

### Synthesis of (S)-12-(4-((((9H-fluoren-9-yl)methoxy)carbonyl)amino)butyl)-11,14-dioxo-4,7,17,20-tetraoxa-10,13-diazatricos-22-ynoic acid

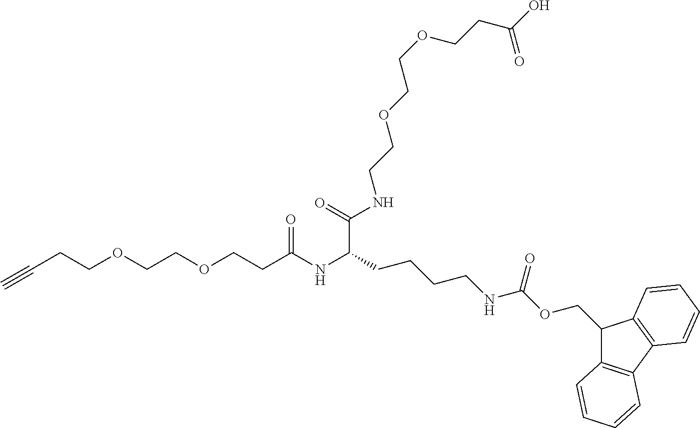 [SMILES: C#CCCOCCOCCC(=O)N[C@@H](CCCCNC(=O)OCC1c2ccccc2-c2ccccc21)C(=O)NCCOCCOCCC(=O)O]

To 3-(2-(prop-2-yn-1-yloxy)ethoxy)propanoic acid (65.5 mg, 0.381 mmol) in DMF (2 ml) was added DIPEA (0.240 ml, 1.37 mmol) and HATU (150 mg, 0.394 mmol). The mixture was stirred at rt for 15 min. Then at 0° C. (S)-9-amino-1-(9H-fluoren-9-yl)-3,10-dioxo-2,14,17-trioxa-4,11-diazaicosan-20-oic acid as a TFA salt (220 mg, 0.343 mmol) in DMF (2 ml) was added. The mixture was stirred at rt for 1 h and was diluted with DMSO/water (10/4 mL) and purified by RP-HPLC (C18, ACN/water with 0.1% TFA) to afford, after lyophilization, a white floppy powder as (S)-12-(4-((((9H-fluoren-9-yl)methoxy)carbonyl)amino)butyl)-11,14-dioxo-4,7,17,20-tetraoxa-10,13-diazatricos-22-ynoic acid (117 mg, 50% yield). LC/MS: MH⁺=682.4, Rt=0.97 min (2 min acidic method); HRMS: MH⁺=682.2300, Rt=2.16 min (5 min acidic method).

### Synthesis of (S)-9-amino-1-(9H-fluoren-9-yl)-3,10-dioxo-2,14,17,20-tetraoxa-4,11-diazatricosan-23-oic acid

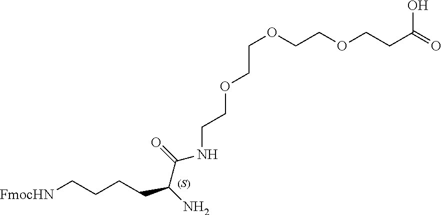

N6-(((9H-fluoren-9-yl)methoxy)carbonyl)-N2-(tert-butoxycarbonyl)-L-lysine (785 mg, 1.68 mmol) and tert-butyl 3-(2-(2-(2-aminoethoxy)ethoxy)ethoxy)propanoate (540 mg, 1.60 mmol) in Acetonitrile (Volume: 12 ml) at 0° C. was DMTMM (422 mg, 1.76 mmol). To the slurry was added DIPEA (0.558 ml, 3.19 mmol). The mixture was stirred at 0° C. for 5 min and then rt for 1.5 h. The solvent was removed by rotary evaporation and under high vac. To the resulting oily material was added CH₂Cl₂ (12 ml), triethylsilane (0.255 ml, 1.60 mmol) and TFA (6.15 ml, 80 mmol) at 0° C.

The mixture was stirred at rt for 1.5 h. The solvent was removed via rotary evaporation and then under high vac. □□The resulting oil was dissolved in DMSO/water and was purified by RP-HPLC (C18, ACN/water with 0.1% TFA) to afford, after lyophilization, a white powder as (S)-9-amino-1-(9H-fluoren-9-yl)-3,10-dioxo-2,14,17,20-tetraoxa-4,11-diazatricosan-23-oic acid as a TFA salt (933 mg, 85% yield). LC/MS: MH⁺=572.4, Rt=0.79 min (acidic, 2 min acidic method). ¹H NMR (400 MHz, DMSO-d6) δ 8.54-8.45 (m, 1H), 8.17 (s, 1H), 8.06 (s, 2H), 7.90 (dt, J=7.6, 0.9 Hz, 2H), 7.68 (d, J=7.5 Hz, 2H), 7.46-7.39 (m, 2H), 7.38-7.31 (m, 2H), 7.30-7.18 (m, 1H), 4.31 (d, J=6.8 Hz, 2H), 4.21 (t, J=6.8 Hz, 1H), 3.94-3.65 (m, 1H), 3.59 (t, J=6.3 Hz, 2H), 3.44 (t, J=5.5 Hz, 8H), 3.38-3.18 (m, 2H), 2.96 (q, J=6.6 Hz, 2H), 2.61 (s, 2H), 2.62-2.52 (m, 2H), 2.51 (s, 2H), 2.43 (t, J=6.3 Hz, 2H), 1.82-1.61 (m, 2H), 1.46-1.15 (m, 4H).

### Synthesis of (S)-15-(4-((((9H-fluoren-9-yl)methoxy)carbonyl)amino)butyl)-14,17-dioxo-4,7,10,20,23-pentaoxa-13,16-diazahexacos-25-ynoic acid

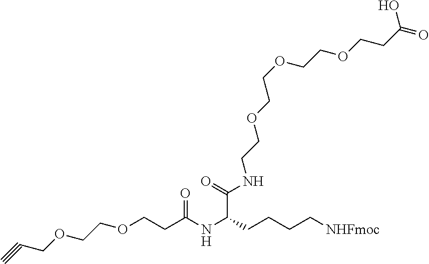

To 3-(2-(prop-2-yn-1-yloxy)ethoxy)propanoic acid (84 mg, 0.49 mmol) in DMF (2.5 ml) was added DIPEA (0.309 ml, 1.77 mmol) and HATU (193 mg, 0.508 mmol). The mixture was stirred at rt for 15 min. Then at 0° C. (S)-9-amino-1-(9H-fluoren-9-yl)-3,10-dioxo-2,14,17,20-tetraoxa-4,11-diazatricosan-23-oic acid as a TFA salt (303 mg, 0.442 mmol) in DMF (2.5 ml) was added. The mixture was stirred at rt for 1 h and was diluted with DMSO/water (10/4 mL) and purified by RP-HPLC (C18, ACN/water with 0.1% TFA) to afford, after lyophilization, a white floppy powder as (S)-15-(4-((((9H-fluoren-9-yl)methoxy)carbonyl)amino)butyl)-14,17-dioxo-4,7,10,20,23-pentaoxa-13,16-diazahexacos-25-ynoic acid (206 mg, 64% yield). HRMS: MH⁺=726.3700, Rt=2.19 min (5 min acidic method).

### Synthesis of tert-butyl 1-(9H-fluoren-9-yl)-11-(2-hydroxyethyl)-3,10-dioxo-2,7,14,17,20-pentaoxa-4,11-diazatricosan-23-oate

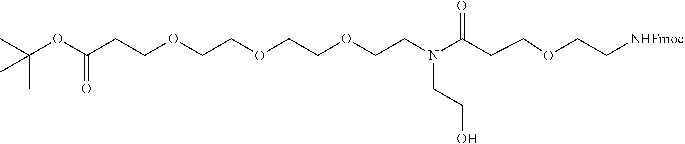

In a 10 mL glass vial equipped with magnetic stir bar, to tert-butyl 3-(2-(2-(2-(tosyloxy)ethoxy)ethoxy)ethoxy)propanoate (311 mg, 0.719 mmol) dissolved in EtOH (1 mL) was added Ethanoamine (220 mg, 3.60 mmol). The reaction mixture was stirred at 75° C. for 2 h, cooled to RT. The solvent was removed under reduced pressure and dried under high vac overnight to afford a thick oil (515 mg). To this thick oil (412 mg) was added at 0° C. 3-(2-((((9H-fluoren-9-yl)methoxy)carbonyl)amino)ethoxy)propanoic acid (1329 mg, 3.74 mmol) in Acetonitrile (6 mL) and THF (3 mL), DMTMM (1070 mg, 3.86 mmol) and DIPEA (0.800 mL, 4.60 mmol). The mixture was stirred at rt for 1 h. The cloudy mixture was filtered. The filtrate was concentrated and purified by flash chromatography (0-10% MeOH in CH₂Cl₂ elution) twice to provide, after concentration of appropriate fractions, tert-butyl 1-(9H-fluoren-9-yl)-11-(2-hydroxyethyl)-3,10-dioxo-2,7,14,17,20-pentaoxa-4,11-diazatricosan-23-oate as a white solid (194 mg, yield 51.5%). LC/MS: MH⁺=659.5, Rt=1.13 min (2 min acidic method). ¹H NMR (400 MHz, DMSO-d6) δ 7.89 (d, J=7.5 Hz, 2H), 7.70 (d, J=7.4 Hz, 2H), 7.45-7.38 (m, 2H), 7.37-7.25 (m, 3H), 4.69 (d, J=64.3 Hz, 1H), 4.41-4.01 (m, 4H), 3.88 (d, J=33.5 Hz, 3H), 3.64-3.55 (m, 4H), 3.54-3.49 (m, 4H), 3.46-3.28 (m, 10H), 3.20-3.08 (m, 5H), 2.64-2.56 (m, 2H), 2.44-2.37 (m, 2H), 1.39 (d, J=1.9 Hz, 9H).

### Synthesis of 11-(2-(((N-(2-(2-(2-carboxyethoxy)ethoxy)ethyl)sulfamoyl)carbamoyl)oxy)ethyl)-1-(9H-fluoren-9-yl)-3,10-dioxo-2,7,14,17,20-pentaoxa-4,11-diazatricosan-23-oic acid

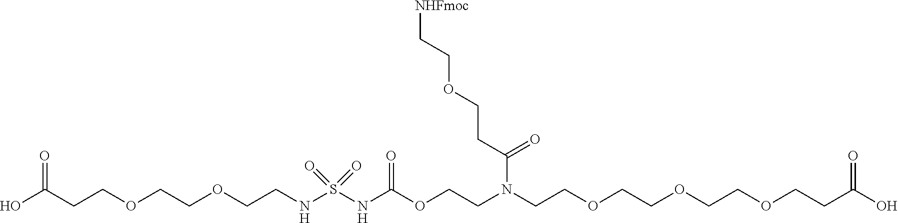

To tert-butyl 1-(9H-fluoren-9-yl)-11-(2-hydroxyethyl)-3,10-dioxo-2,7,14,17,20-pentaoxa-4,11-diazatricosan-23-oate (97 mg, 0.147 mmol) in CH₂Cl₂ (2.5 ml) was added sulfurisocyanatidic chloride (0.013 ml, 0.15 mmol) at 0° C. The mixture was stirring at 0° C. for 30 min. Then TEA (0.103 ml, 0.736 mmol) and tert-butyl 3-(2-(2-aminoethoxy)ethoxy)propanoate (37.8 mg, 0.162 mmol) in CH₂Cl₂ (1.2 mL) were added. The mixture was stirred at 0° C. for 1 h, then rt for 1 h and was quenched with satd. NH₄Cl, and 1 N HCl (0.9 mL). The aqueous was extracted with CH₂Cl₂ (5×). The organic layer was dried over anh, Na₂SO₄, filtered and concentrated via rotary evaporation to provide a clear oil. Purification was conducted by flash chromatography (0-100% EtOAc in heptane, then 0-15% MeOH in CH₂Cl₂) to afford the di-t-butyl ester of the title compound as a clear oil (25 mg, 17% yield). LC/MS: MH⁺=997.6, Rt=1.38 min. HR/MS (peptide, 5 min): 3.01 min, M+=997.4900, 100% pure. ¹H NMR (400 MHz, Methylene Chloride-d2) δ 7.86-7.78 (m, 2H), 7.69 (t, J=7.9 Hz, 2H), 7.49-7.40 (m, 2H), 7.40-7.31 (m, 2H), 6.41-6.13 (m, 1H), 6.04-5.50 (m, 1H), 4.50-4.21 (m, 5H), 3.92-3.66 (m, 9H), 3.66-3.49 (m, 20H), 3.44-3.22 (m, 4H), 2.55-2.45 (m, 4H), 1.47 (s, 18H). To the di-ester in CH₂Cl₂ (1.3 ml) at 0° C. was added triethylsilane (0.004 ml, 0.03 mmol) and TFA (0.567 ml, 7.36 mmol). The mixture was stirred at rt for 1 h and then concentrated via rotary evaporation. The residue was dried in vacuo for 30 min and then lyophilized (water/ACN=3/3 mL) to afford 11-(2-(((N-(2-(2-(2-carboxyethoxy)ethoxy)ethyl)sulfamoyl)carbamoyl)oxy)ethyl)-1-(9H-fluoren-9-yl)-3,10-dioxo-2,7,14,17,20-pentaoxa-4,11-diazatricosan-23-oic acid as a white solid (24 mg). HRMS: MH⁺=885.3500, Rt=2.05 min (5 min acidic method). ¹H NMR (400 MHz, DMSO) δ 12.12 (s, 2H), 7.89 (d, J=7.5 Hz, 2H), 7.69 (d, J=7.5 Hz, 2H), 7.46-7.39 (m, 2H), 7.37-7.30 (m, 2H), 7.28 (t, J=5.2 Hz, 1H), 4.33-4.08 (m, 5H), 3.67-3.55 (m, 7H), 3.55-3.41 (m, 20H), 3.41-3.36 (m, 2H), 3.18-2.99 (m, 4H), 2.69-2.56 (m, 2H), 2.47-2.39 (m, 5H).

### Synthesis of benzyl 3-(2-(2-(2-aminoethoxy)ethoxy)ethoxy)propanoate

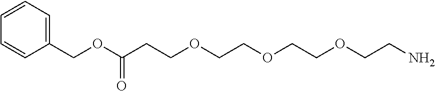 [SMILES: NCCOCCOCCOCCC(=O)OCc1ccccc1]

To benzyl 2,2-dimethyl-4-oxo-3,8,11,14-tetraoxa-5-azaheptadecan-17-oate (1000 mg, 2.430 mmol) in CH₂Cl₂ (24 mL) was added triethylsilane (0.388 mL, 2.43 mmol) and TFA (8 mL, 0.1 mol). The mixture was stirred at rt for 1 h. The volatile was removed via rotary evaporation and under high vac for 15 min, then diluted with ACN/water (5/5 mL) and lyophilized to give benzyl 3-(2-(2-(2-aminoethoxy)ethoxy)ethoxy)propanoate as a TFA salt form as a colorless oil (1.269 g). LC/MS: MH⁺=312.3, Rt=0.61 min (2 min acidic method). ¹H NMR (400 MHz, DMSO) δ 7.74 (s, 3H), 7.43-7.28 (m, 5H), 5.11 (s, 2H), 3.66 (t, J=6.2 Hz, 2H), 3.60-3.54 (m, 6H), 3.50 (s, 4H), 2.97 (h, J=5.7 Hz, 2H), 2.61 (t, J=6.2 Hz, 2H).

### Synthesis of benzyl (S)-10-amino-2,2-dimethyl-4,11-dioxo-3,15,18,21-tetraoxa-5,12-diazatetracosan-24-oate

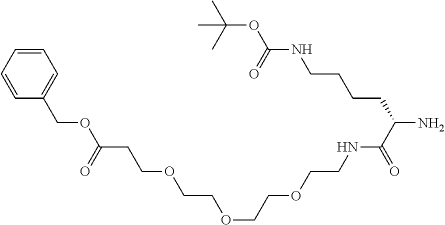 [SMILES: CC(C)(C)OC(=O)NCCCC[C@H](N)C(=O)NCCOCCOCCOCCC(=O)OCc1ccccc1]

To a mixture of benzyl 3-(2-(2-(2-aminoethoxy)ethoxy)ethoxy)propanoate as a TFA salt (707 mg, 1.66 mmol) and Fmoc-Lys(Boc)-OSu (987 mg, 1.75 mmol) in DMF (6 mL) was added DIPEA (0.871 mL, 4.99 mmol). The mixture was stirred at rt overnight. To the mixture was added dimethylamine (2N in THF, 4.15 mL, 8.31 mmol). The mixture was stirred at rt for 2 h, diluted with DMSO (10 mL) and purified by RP-HPLC (C18, ACN/water with 0.1% TFA) to afford, after lyophilization of the appropriate fractions, benzyl (S)-10-amino-2,2-dimethyl-4,11-dioxo-3,15,18,21-tetraoxa-5,12-diazatetracosan-24-oate as a TFA salt form as a clear thick oil (630 mg, 58% yield). LC/MS: MH⁺=540.5, Rt=0.83 min (2 min acidic method). HRMS: MH⁺=540.3300, Rt=1.74 min (5 min acidic method). ¹H NMR (400 MHz, DMSO) δ 8.48 (t, J=5.6 Hz, 1H), 8.05 (d, J=4.8 Hz, 3H), 7.43-7.26 (m, 5H), 6.76 (t, J=5.7 Hz, 1H), 5.11 (s, 2H), 3.73-3.59 (m, 5H), 3.48-3.17 (m, 10H), 2.93-2.84 (m, 2H), 2.61 (t, J=6.2 Hz, 2H), 1.71-1.61 (m, 2H), 1.37 (s, 11H), 1.31-1.19 (m, 2H).

### Synthesis of 1-benzyl 29-methyl (S)-15-(4-((tert-butoxycarbonyl)amino)butyl)-14,17-dioxo-4,7,10,20,23,26-hexaoxa-13,16-diazanonacosanedioate

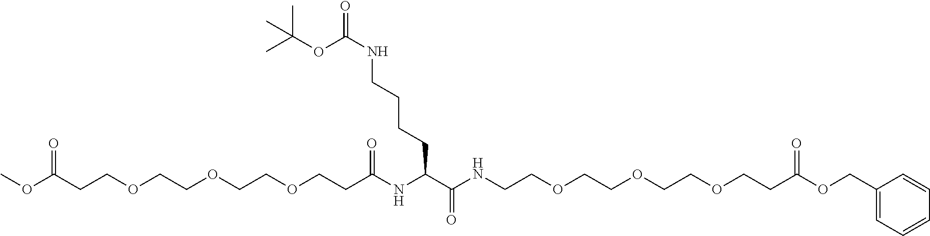 [SMILES: COC(=O)CCOCCOCCOCCC(=O)N[C@@H](CCCCNC(=O)OC(C)(C)C)C(=O)NCCOCCOCCOCCC(=O)OCc1ccccc1]

To benzyl (S)-10-amino-2,2-dimethyl-4,11-dioxo-3,15,18,21-tetraoxa-5,12-diazatetracosan-24-oate as a TFA salt form (630 mg, 1.17 mmol) and 3-oxo-2,6,9,12-tetraoxapentadecan-15-oic acid (324 mg, 1.226 mmol) in Acetonitrile (8 ml) at) at 0° C. was added DMTMM (303 mg, 1.26 mmol) and DIPEA (0.816 ml, 4.67 mmol). The mixture was stirred at for 1.5 h. The volatile was removed via rotary evaporation and then in high vac for 15 min. The resulting colorless oil was purified by flash chromatography (0-10% MeOH in CH₂Cl₂) to provide 1-benzyl 29-methyl (S)-15-(4-((tert-butoxycarbonyl)amino)butyl)-14,17-dioxo-4,7,10,20,23,26-hexaoxa-13,16-diazanonacosanedioate as a colorless oil (551 mg, yield 61%). LC/MS: MH⁺=786.4, Rt=1.02 min (2 min acidic method). ¹H NMR (400 MHz, DMSO) δ 7.94-7.85 (m, 2H), 7.41-7.28 (m, 5H), 6.72 (t, J=5.7 Hz, 1H), 5.11 (s, 2H), 4.23-4.11 (m, 1H), 3.70-3.55 (m, 9H), 3.48 (t, J=3.0 Hz, 16H), 3.38 (t, J=6.0 Hz, 2H), 3.26-3.10 (m, 2H), 2.91-2.81 (m, 2H), 2.58 (dd, J=26.8, 6.2 Hz, 4H), 2.45-2.29 (m, 2H), 1.37 (m, 15H).

### Synthesis of (S)-17-(4-((tert-butoxycarbonyl)amino)butyl)-3,15,18-trioxo-2,6,9,12,22,25,28-heptaoxa-16,19-diazahentriacontan-31-oic acid

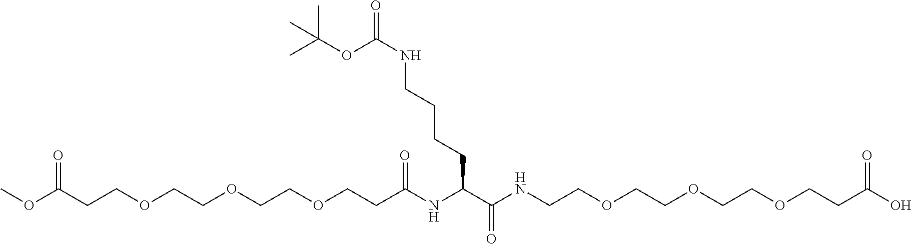 [SMILES: COC(=O)CCOCCOCCOCCC(=O)N[C@@H](CCCCNC(=O)OC(C)(C)C)C(=O)NCCOCCOCCOCCC(=O)O]

A mixture of 1-benzyl 29-methyl (S)-15-(4-((tert-butoxycarbonyl)amino)butyl)-14,17-dioxo-4,7,10,20,23,26-hexaoxa-13,16-diazanonacosanedioate (202 mg, 0.257 mmol) and Pd—C(10% on carbon, 27 mg, 0.026 mmol) in THF (10 mL) was purged with H₂ three times and then stirred vigorously under balloon hydrogen for 3.5 h. The mixture was filtered and washed first with CH₂Cl₂ and then with 1:1 CH₂Cl₂/MeOH (20 mL). The filtrate was concentrated via rotary evaporation and then under high vac overnight to afford (S)-17-(4-((tert-butoxycarbonyl)amino)butyl)-3,15,18-trioxo-2,6,9,12,22,25,28-heptaoxa-16,19-diazahentriacontan-31-oic acid as a thick oil (180 mg, 100% yield). LC/MS: MH⁺=696.3, Rt=0.79 min (2 min acidic method). LC/MS: MH⁺=696.8, Rt=1.64 min (5 min acidic method). ¹H NMR (400 MHz, DMSO) δ 12.13 (s, 1H), 7.90 (q, J=7.0 Hz, 2H), 6.72 (d, J=5.7 Hz, 1H), 4.26-4.11 (m, 1H), 3.66-3.54 (m, 9H), 3.54-3.42 (m, 16H), 3.39 (t, J=5.9 Hz, 2H), 3.26-3.11 (m, 2H), 2.91-2.81 (m, 2H), 2.57-2.52 (m, 2H), 2.47-2.28 (m, 4H), 1.63-1.14 (m, 15H).

### Synthesis of benzyl 3-(2-(2-aminoethoxy)ethoxy)propanoate

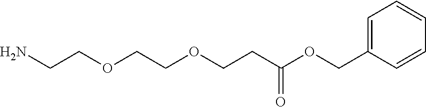 [SMILES: NCCOCCOCCC(=O)OCc1ccccc1]

To benzyl 2,2-dimethyl-4-oxo-3,8,11-trioxa-5-azatetradecan-14-oate (1110 mg, 3.02 mmol) in CH₂Cl₂ (24 mL) was added triethylsilane (0.483 mL, 3.02 mmol) and TFA (8 mL, 0.1 mol). The mixture was stirred at rt for 1 h. The volatile was removed via rotary evaporation and under high vac for 15 min, then diluted with ACN/water (5/5 mL) and lyophilized to give benzyl 3-(2-(2-aminoethoxy)ethoxy)propanoate as a TFA salt form as a light yellow oil (1.132 g, yield 98%). LC/MS: MH⁺=268.2, Rt=0.58 min (2 min acidic method).

### Synthesis of benzyl (S)-10-amino-2,2-dimethyl-4,11-dioxo-3,15,18-trioxa-5,12-diazahenicosan-21-oate

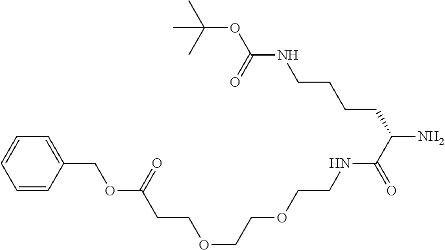 [SMILES: CC(C)(C)OC(=O)NCCCC[C@H](N)C(=O)NCCOCCOCCC(=O)OCc1ccccc1]

To a benzyl 3-(2-(2-(2-aminoethoxy)ethoxy)ethoxy)propanoate as a TFA salt form (1132 mg, 2.97 mmol) and Fmoc-Lys(Boc)-OSu (1763 mg, 3.12 mmol) in DMF (10 mL) was added DIPEA (1.56 ml, 8.91 mmol). The mixture was stirred at rt overnight. To the mixture was added dimethylamine (2N in THF, 7.42 ml, 14.8 mmol). The mixture was stirred at rt for 2 h, diluted with DMSO (15 mL) and purified by RP-HPLC (C18, ACN/water with 0.1% TFA) to afford, after lyophilization of the appropriate fractions, benzyl (S)-10-amino-2,2-dimethyl-4,11-dioxo-3,15,18-trioxa-5,12-diazahenicosan-21-oate as a TFA salt form as a clear thick oil (947 mg, yield 52%). LC/MS: MH⁺=496.5, Rt=0.81 min (2 min acidic method). ¹H NMR (400 MHz, DMSO) δ 8.48 (t, J=5.6 Hz, 1H), 8.12-8.00 (m, 3H), 7.41-7.29 (m, 5H), 6.76 (t, J=5.7 Hz, 1H), 5.11 (s, 2H), 3.75-3.63 (m, 4H), 3.51 (s, 3H), 3.44 (t, J=5.6 Hz, 2H), 3.38-3.17 (m, 2H), 2.94-2.82 (m, 2H), 2.61 (t, J=6.2 Hz, 2H), 1.71-1.61 (m, 2H), 1.37 (m, 13H).

### Synthesis of 1-benzyl 29-methyl (S)-12-(4-((tert-butoxycarbonyl)amino)butyl)-11,14-dioxo-4,7,17,20,23,26-hexaoxa-10,13-diazanonacosanedioate

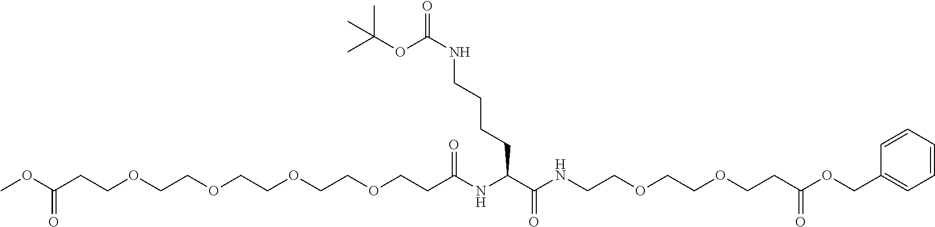 [SMILES: COC(=O)CCOCCOCCOCCOCCC(=O)N[C@@H](CCCCNC(=O)OC(C)(C)C)C(=O)NCCOCCOCCC(=O)OCc1ccccc1]

To benzyl (S)-10-amino-2,2-dimethyl-4,11-dioxo-3,15,18-trioxa-5,12-diazahenicosan-21-oate as a TFA salt form (1043 mg, 1.71 mmol) and 3-oxo-2,6,9,12,15-pentaoxaoctadecan-18-oic acid (501 mg, 1.624 mmol). in Acetonitrile (10 ml) at) at 0° C. was added DMTMM (401 mg, 1.67 mmol) and DIPEA (1.081 ml, 6.19 mmol). The mixture was stirred at for 2.5 h. To the mixture were added 3-oxo-2,6,9,12,15-pentaoxaoctadecan-18-oic acid (150 mg, 0.487 mmol) and DMTMM (120 mg, 0.501 mmol) and DIPEA (0.324 ml, 1.86 mmol). The mixture was stirred at rt for 30 min. The volatile was removed via rotary evaporation and then in high vac for 15 min. The resulting colorless oil was purified by flash chromatography (0-10% MeOH in CH₂Cl₂), followed by washing of the product thereof with EtOAc with 1:1 satd. NaHCO₃/H₂O, to provide 1-benzyl 29-methyl (S)-12-(4-((tert-butoxycarbonyl)amino)butyl)-11,14-dioxo-4,7,17,20,23,26-hexaoxa-10,13-diazanonacosanedioate as a white solid (1062 mg, yield 79%). LC/MS: MH⁺=786.7, Rt=1.04 min (2 min acidic method). ¹H NMR (400 MHz, DMSO) δ 7.95-7.84 (m, 2H), 7.42-7.29 (m, 5H), 6.72 (t, J=5.7 Hz, 1H), 5.11 (s, 2H), 4.25-4.14 (m, 1H), 3.70-3.55 (m, 9H), 3.53-3.44 (m, 16H), 3.38 (t, J=6.0 Hz, 2H), 3.25-3.13 (m, 2H), 2.91-2.81 (m, 2H), 2.58 (dd, J=26.1, 6.2 Hz, 4H), 2.45-2.29 (m, 2H), 1.37 (m, 16H).

### Synthesis of (S)-20-(4-((tert-butoxycarbonyl)amino)butyl)-3,18,21-trioxo-2,6,9,12,15,25,28-heptaoxa-19,22-diazahentriacontan-31-oic acid

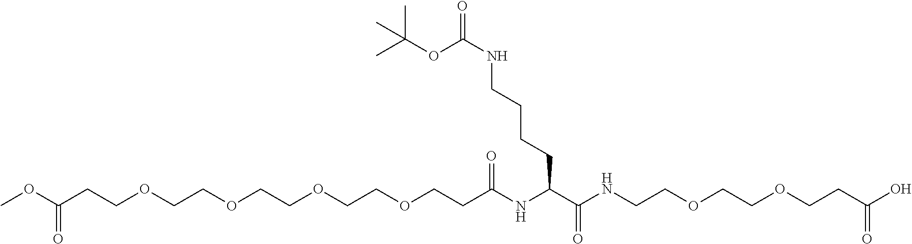 [SMILES: COC(=O)CCOCCOCCOCCOCCC(=O)N[C@@H](CCCCNC(=O)OC(C)(C)C)C(=O)NCCOCCOCCC(=O)O]

A mixture of 1-benzyl 29-methyl (S)-12-(4-((tert-butoxycarbonyl)amino)butyl)-11,14-dioxo-4,7,17,20,23,26-hexaoxa-10,13-diazanonacosanedioate (620 mg, 789 μmol) and Pd—C(10% on carbon, 84 mg, 0.079 mmol) in THF (30 mL) was purged with H₂ three times and then stirred vigorously under balloon hydrogen for 3.5 h. The mixture was filtered and washed first with CH₂Cl₂ and then with 1:1 CH₂Cl₂/MeOH (60 mL). The filtrate was concentrated via rotary evaporation and then under high vac overnight to afford (S)-20-(4-((tert-butoxycarbonyl)amino)butyl)-3,18,21-trioxo-2,6,9,12,15,25,28-heptaoxa-19,22-diazahentriacontan-31-oic acid (620 mg, 789 μmol). LC/MS: MH⁺=696.7, Rt=1.08 min (2 min basic method). ¹H NMR (400 MHz, DMSO) δ 12.14 (s, 1H), 7.96-7.82 (m, 2H), 6.73 (t, J=5.6 Hz, 1H), 4.26-4.14 (m, 1H), 3.67-3.54 (m, 9H), 3.52-3.44 (m, 15H), 3.39 (t, J=6.0 Hz, 2H), 3.27-3.10 (m, 2H), 2.92-2.81 (m, 2H), 2.55 (d, J=6.2 Hz, 3H), 2.47-2.34 (m, 4H), 1.37 (m, 15H).

### Synthesis of tert-butyl (2-(3-oxo-3-((2-(2,2,2-trifluoroacetamido)ethyl)(2-(tritylamino)ethyl)amino)propoxy)ethyl)carbamate

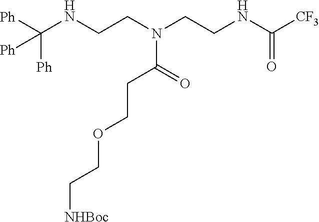 [SMILES: CC(C)(C)OC(=O)NCCOCCC(=O)N(CCNC(=O)C(F)(F)F)CCNC(c1ccccc1)(c1ccccc1)c1ccccc1]

To 2,2,2-trifluoro-N-(2-((2-(tritylamino)ethyl)amino)ethyl)acetamide (14.061 g, 31.85 mmol, J. Org. Chem. 2012, 77, 4226-4234) and 3-(2-((tert-butoxycarbonyl)amino)ethoxy)propanoic acid (7.949 g, 34.08 mmol) in CH₂Cl₂ (600 mL) at 0° C. was added in 5 portions over 20 min EDC (7.326 g, 38.22 mmol). The clear solution was stirred at 0° C. for 30 min, then warmed up to rt overnight. To the mixture was added, more 3-(2-((tert-butoxycarbonyl)amino)ethoxy)propanoic acid (0.556 g, 2.38 mmol) and EDC (0.733 g, 3.82 mmol). The mixture was stirred at rt for 4 h, and was concentrated to ca. 400 mL and was washed with 1:1 brine/water (150 mL). The aqueous was extracted with CH₂Cl₂ (20 mL). The washing and back extraction procedure was repeated three more times. The combined organic phase was washed with satd. NaHCO₃ (50 mL×2), brine, dried over anh. Na₂SO₄, filtered and concentrated to provide tert-butyl (2-(3-oxo-3-((2-(2,2,2-trifluoroacetamido)ethyl)(2-(tritylamino)ethyl)amino)propoxy)ethyl)carbamate as a white foam (19.98 g, yield 90%). LC/MS: M+Na⁺=679.5, M-=655.5, Rt=1.18 min. (2 min acidic). The product was used directly in the next step without purification.

### Synthesis of tert-butyl (2-(3-((2-aminoethyl)(2-(tritylamino)ethyl)amino)-3-oxopropoxy)ethyl)carbamate

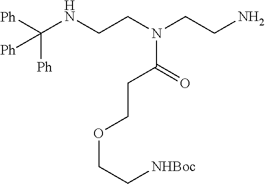 [SMILES: CC(C)(C)OC(=O)NCCOCCC(=O)N(CCN)CCNC(c1ccccc1)(c1ccccc1)c1ccccc1]

To tert-butyl (2-(3-oxo-3-((2-(2,2,2-trifluoroacetamido)ethyl)(2-(tritylamino)ethyl)amino)propoxy)ethyl)carbamate (9.210, 14.02 mmol) dissolved in MeOH (130 mL) and water (13 mL) was added K₂CO₃ (9.69 g, 70.1 mmol). The slurry was stirred at rt over weekend and then was filtered and washed with EtOAc. The filtrate was concentrated. The residue was washed with 1:1 brine/water (80 mL). The aqueous was extracted with EtOAc (3×). The combined organic layer was washed with brine, dried over anh. Na₂SO₄, filtered and concentrated. To remove the residual solvent, the product was dissolved with CH₂Cl₂ and concentrated. This was repeated one more time to afford tert-butyl (2-(3-((2-aminoethyl)(2-(tritylamino)ethyl)amino)-3-oxopropoxy)ethyl)carbamate, following drying under high vac, as a white foaming (7.26 g, 92% yield). LC/MS: MH⁺=561.6, Rt=2.19 min. (5 min acidic method). ¹H NMR (400 MHz, DMSO) δ 7.45-7.35 (m, 6H), 7.33-7.25 (m, 6H), 7.24-7.13 (m, 3H), 6.72 (q, J=6.3 Hz, 1H), 3.57 (m, J=13.0, 6.6 Hz, 2H), 3.43-3.25 (m, 11H, overlapping with water), 3.22-2.94 (m, 4H), 2.80-2.53 (m, 4H), 2.47-2.23 (m, 2H), 2.18-2.02 (m, 2H), 1.37 (d, J=1.3 Hz, 9H). The product was used directly in the next step without purification.

### Synthesis of 3-(2-(3-(allyloxy)-3-oxopropoxy)ethoxy)propanoic acid

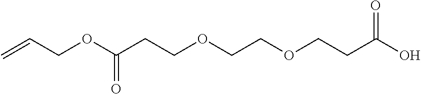 [SMILES: C=CCOC(=O)CCOCCOCCC(=O)O]

To a mixture of 3,3′-(ethane-1,2-diylbis(oxy))dipropionic acid (7.720 g, 37.44 mmol), prop-2-en-1-ol (2.175 g, 2.55 mL, 37.4 mmol) and DMAP (457 mg, 3.74 mmol) in CH₂Cl₂ (66 mL) and dioxane (100.0 mL) at 0° C. was added dropwise, dicyclohexylmethanediimine (8.112 g, 39.31 mmol) slurry in dichloromethane (34 mL) over 20 min. The mixture was warmed up to rt and stirred overnight. The reaction mixture was filtered and the solid was washed with CH₂Cl₂ (100 mL). The volatile from the filtrate was removed under reduced pressure. The resulting oil was mixed with DMSO (24 mL) and filtered. The filtrate was purified by RP-HPLC (C18, ACN/water with 0.1% TFA), to provide, after lyophilization of the appropriate fractions, 3-(2-(3-(allyloxy)-3-oxopropoxy)ethoxy)propanoic acid as a colorless oil (4.487 g, 49% yield). LC/MS: MH⁺=247.0, Rt=0.66 min (2 min acidic method)¹H NMR (400 MHz, DMSO) δ 5.97-5.84 (m, 1H), 5.34-5.26 (m, 1H), 5.24-5.17 (m, 1H), 4.59-4.54 (m, 2H), 3.67-3.56 (m, 4H), 3.52-3.30 (m, 6H, overlapping with DMSO), 2.57 (t, J=6.2 Hz, 2H), 2.43 (t, J=6.4 Hz, 2H).

### Synthesis of allyl 2,2-dimethyl-4,11,16-trioxo-12-(2-(tritylamino)ethyl)-3,8,19,22-tetraoxa-5,12,15-triazapentacosan-25-oate

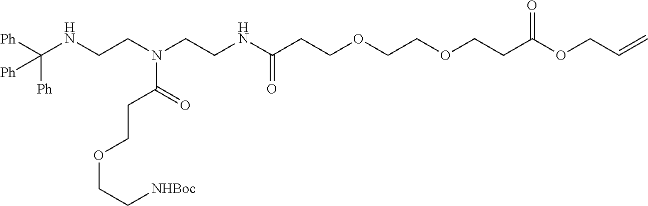 [SMILES: C=CCOC(=O)CCOCCOCCC(=O)NCCN(CCNC(c1ccccc1)(c1ccccc1)c1ccccc1)C(=O)CCOCCNC(=O)OC(C)(C)C]

To tert-butyl (2-(3-((2-aminoethyl)(2-(tritylamino)ethyl)amino)-3-oxopropoxy)ethyl)carbamate (8420 mg, 15.02 mmol) and 3-(2-(3-(allyloxy)-3-oxopropoxy)ethoxy)propanoic acid (3.957 g, 16.07 mmol) dissolved in CH₂Cl₂ (150 mL) at 0° C. was added EDC (3.224 g, 16.82 mmol) in two portions over 10 min. The mixture was warmed up and stirred at rt overnight. To the mixture was added 3-(2-(3-(allyloxy)-3-oxopropoxy)ethoxy)propanoic acid (277 mg, 1.12 mmol), and EDC (226 mg, 1.18 mmol). After being stirred at rt overnight, the mixture was diluted with CH₂Cl₂ (200 mL) and was washed with 1:1 brine/water (200 mL). The aqueous was extracted with CH₂Cl₂ (20 mL). This washing and extraction procedure was repeated three more times. The combined organic phase was washed with satd. NaHCO₃ (200 mL×2), brine, dried over anh. Na₂SO₄, filtered and concentrated to give a colorless oil. This crude product was purified by flash chromatography (0-10% MeOH in CH₂Cl₂) to afford allyl 2,2-dimethyl-4,11,16-trioxo-12-(2-(tritylamino)ethyl)-3,8,19,22-tetraoxa-5,12,15-triazapentacosan-25-oate as a thick colorless oil (11.275 g, 95% yield). LC/MS: MH⁺=789.7, Rt=1.05 min (2 min acidic method). ¹H NMR (400 MHz, DMSO) δ 7.98 (d, J=3.3 Hz, 1H), 7.82 (t, J=6.0 Hz, 1H), 7.40-7.33 (m, 6H), 7.28 (t, J=7.5 Hz, 6H), 7.22-7.14 (m, 3H), 6.71 (d, J=5.8 Hz, 1H), 5.90 (dddd, J=15.8, 9.4, 6.0, 4.6 Hz, 1H), 5.76 (s, 1H), 5.33-5.25 (m, 1H), 5.22-5.16 (m, 1H), 4.58-4.52 (m, 2H), 3.66-3.52 (m, 6H), 3.49-3.35 (m, 6H), 3.27-2.96 (m, 6H), 2.82-2.53 (m, 5H), 2.36-2.20 (m, 3H), 2.15-2.01 (m, 2H), 1.37 (s, 9H).

### Synthesis of allyl 12-(2-aminoethyl)-2,2-dimethyl-4,11,16-trioxo-3,8,19,22-tetraoxa-5,12,15-triazapentacosan-25-oate

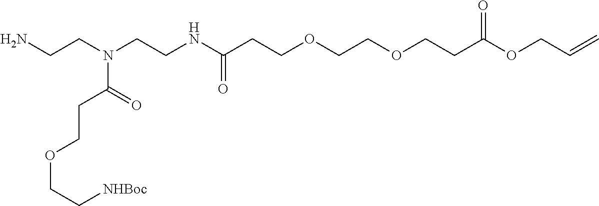 [SMILES: C=CCOC(=O)CCOCCOCCC(=O)NCCN(CCN)C(=O)CCOCCNC(=O)OC(C)(C)C]

To allyl 2,2-dimethyl-4,11,16-trioxo-12-(2-(tritylamino)ethyl)-3,8,19,22-tetraoxa-5,12,15-triazapentacosan-25-oate (8763 mg, 11.11 mmol) dissolved (via sonication) in trifluoroethanol (19 mL) was added acetic acid (19.07 mL, 333.2 mmol). The mixture was stirred at 60° C. for 2 h. The reaction mixture was cooled to rt and mixed with anh. toluene (100 mL) and concentrated. The azeotropical distillation with toluene was repeated two more times. To the residue was added toluene (8 mL) and the resulting solution was added dropwise to ice-cold 1:1 diethyl ether/heptane (400 mL) with rapid stirring. Stirring continued at 0° C. for 15 min. The clear top mother liquid was decanted and discarded. The bottom oily material was washed with fresh 1:1 ether/heptane (2×50 mL) and dried in vacuum to give an oil (7.718 g) as HOAc salt of the title compound. The material was dissolved with 1/9 TFE/CH₂Cl₂ (250 mL) and was washed with satd. NaHCO₃ (2×100 mL) until the aqueous was basic. The combined organic phase was washed with brine (75 mL). The aqueous was extracted with 1/9 TFE/CH₂Cl₂ (20 mL) twice. The combined organic phase was dried over anh. Na₂SO₄, filtered and concentrated to give a colorless oil as allyl 12-(2-aminoethyl)-2,2-dimethyl-4,11,16-trioxo-3,8,19,22-tetraoxa-5,12,15-triazapentacosan-25-oate (7.962 g, crude, 76.5% pure by wt. assuming 100% yield) used directly in the next step. LC/MS: MH⁺=547.5, Rt=0.73 min (2 min acidic method).

### Synthesis of 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid

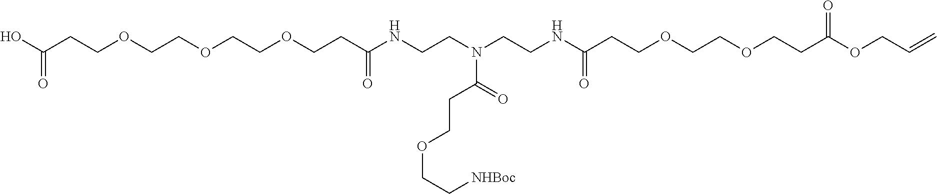 [SMILES: C=CCOC(=O)CCOCCOCCC(=O)NCCN(CCNC(=O)CCOCCOCCOCCC(=O)O)C(=O)CCOCCNC(=O)OC(C)(C)C]

To allyl 12-(2-aminoethyl)-2,2-dimethyl-4,11,16-trioxo-3,8,19,22-tetraoxa-5,12,15-triazapentacosan-25-oate prepared above (665 mg, 76.3% by Wt., 928 μmol) was added 3-(2-(2-(3-((2,5-dioxopyrrolidin-1-yl)oxy)-3-oxopropoxy)ethoxy)ethoxy)propanoic acid (387 mg, 1.11 mmol) dissolved in Acetonitrile (3.7 mL), and DIPEA (323 μL, 1.86 mmol). The mixture was stirred at rt/20 h, The volatile was removed via rotary evaporation. The resulting oil was diluted with DMSO (6 mL) and was purified by RP-HPLC (C18, ACN/water with 0.1% NH₄0H). The desired product-containing fractions (total ca. 112 mL) were diluted with EtOAc (75 mL) and acidified with 1 N HCl (3.5 mL). The organic phase was separated. The aqueous was extracted with EtOAc (2×30 mL). The combined EtOAc extract was mixed with heptane (20 mL) and the mixture was washed with brine (2×15 mL), dried over anh. Na₂SO₄, concentrated to provide a colorless oil as 17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid (274 mg, 37.8% yield). LC/MS: MH⁺=779.7, Rt=1.72 min (5 min acidic method). ¹H NMR (400 MHz, DMSO) δ 12.06 (s, 1H), 8.05-7.94 (m, 1H), 7.93-7.80 (m, 1H), 6.71 (d, J=6.0 Hz, 1H), 5.98-5.84 (m, 1H), 5.35-5.26 (m, 1H), 5.24-5.16 (m, 1H), 4.60-4.51 (m, 2H), 3.68-3.53 (m, 10H), 3.53-3.41 (m, 12H), 3.40-3.24 (m, 8H, overlapping with DMSO), 3.17 (dt, J=16.1, 6.3 Hz, 4H), 3.09-3.00 (m, 2H), 2.61-2.53 (m, 4H), 2.44 (t, J=6.3 Hz, 2H), 2.33-2.24 (m, 4H), 1.37 (s, 9H). The aqueous layer was further extracted with 1/9 TFE/CH₂Cl₂ (3×15 mL) to provide additional title compound as a colorless oil (122 mg, 16.9% yield).

### Synthesis of di-tert-butyl 13-(3-(2-((((9H-fluoren-9-yl)methoxy)carbonyl)amino)ethoxy)propanoyl)-4,7,10,16,19,22-hexaoxa-13-azapentacosanedioate

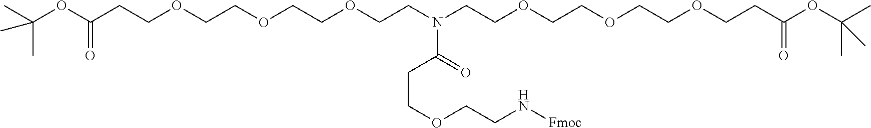

To a solution of 3-(2-((((9H-fluoren-9-yl)methoxy)carbonyl)amino)ethoxy)propanoic acid (CAS number: 1654740-73-4) (87 mg, 0.244 mmol) in DMF (0.50 mL) was added HATU (93 mg, 0.244 mmol) and DIPEA (0.205 mL, 1.172 mmol). The mixture was stirred at RT for 15 min and added into a solution of di-tert-butyl 4,7,10,16,19,22-hexaoxa-13-azapentacosanedioate (Broadpharm) (105 mg, 0.195 mmol) in DMF (0.2 mL). The mixture was stirred at RT for 1 hr and concentrated to remove the volatile. The residue was diluted with DMSO (2 ml) and was purified by RP—ISCO chromatography (ISCO Gold C18 Column, 100 g, Water/MeCN as eluent, 0.1% TFA as modifier) to afford di-tert-butyl 13-(3-(2-((((9H-fluoren-9-yl)methoxy)carbonyl)amino)ethoxy)propanoyl)-4,7,10,16,19,22-hexaoxa-13-azapentacosanedioate (135 mg, 79% yield) after lyophilization. LC/MS: MH⁺=875.8, Rt=1.33 min (2 min acidic method).

### Synthesis of 13-(3-(2-((((9H-fluoren-9-yl)methoxy)carbonyl)amino)ethoxy)propanoyl)-4,7,10,16,19,22-hexaoxa-13-azapentacosanedioic acid

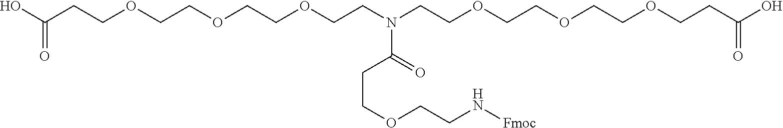

A mixture of di-tert-butyl 13-(3-(2-((((9H-fluoren-9-yl)methoxy)carbonyl)amino)ethoxy)propanoyl)-4,7,10,16,19,22-hexaoxa-13-azapentacosanedioate (135 mg, 0.154 mmol) and TFA (1.010 mL, 13.11 mmol) in DCM (2 mL) was stirred at RT for 2 hr. After adding DCE (3 ml), the mixture was concentrated on rotatory evaporator. The residue was diluted with DMSO (2 mL) and purified by RP—ISCO chromatography (100 G ISCO Gold C18 Column, Water/MeCN as eluent, 0.1% TFA as modifier) to afford 13-(3-(2-((((9H-fluoren-9-yl)methoxy)carbonyl)amino)ethoxy)propanoyl)-4,7,10,16,19,22-hexaoxa-13-azapentacosanedioic acid (120 mg, 99% yield) after lyophilization. LC/MS: MH⁺=763.5, Rt=0.97 min (2 min acidic method).

### Synthesis of tert-butyl (3-(bis(2-(2-(prop-2-yn-1-yloxy)ethoxy)ethyl)amino)-3-oxopropyl)carbamate

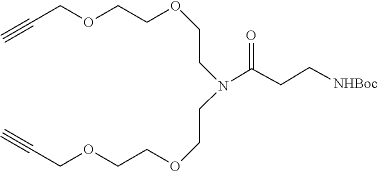 [SMILES: C#CCOCCOCCN(CCOCCOCC#C)C(=O)CCNC(=O)OC(C)(C)C]

To the solution of bis(2-(2-(prop-2-yn-1-yloxy)ethoxy)ethyl)amine (200 mg, 0.743 mmol), 3-((tert-butoxycarbonyl)amino)propanoic acid (148 mg, 0.780 mmol) in DMF (4 ml) was added HATU (296 mg, 0.780 mmol) and then DIPEA (389 μl, 2.228 mmol). The reaction mixture was stirred at RT for 1 h and was purified by a 150 g C18 RP column (Water/MeCN as eluent, 0.1% TFA as modifier) to afford after lyophilization tert-butyl (3-(bis(2-(2-(prop-2-yn-1-yloxy)ethoxy)ethyl)amino)-3-oxopropyl)carbamate (148 mg, 44% yield). LC/MS: MH⁺=441.3, Rt=0.92 min (2 min acidic method).

### Synthesis of 3-amino-N,N-bis(2-(2-(prop-2-yn-1-yloxy)ethoxy)ethyl)propanamide

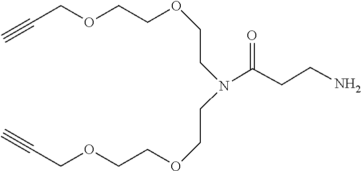 [SMILES: C#CCOCCOCCN(CCOCCOCC#C)C(=O)CCN]

TFA (0.568 mL, 7.38 mmol) was added to a solution of tert-butyl (3-(bis(2-(2-(prop-2-yn-1-yloxy)ethoxy)ethyl)amino)-3-oxopropyl)carbamate (65 mg, 0.15 mmol) in dichloromethane (0.6 mL). The mixture was stirred at RT for 1 hr. After adding DCE (3 ml), the mixture was concentrated on rotatory evaporator. 2-3 ml ether was added to the residue and sonicated, and the ether was removed to afford 3-amino-N,N-bis(2-(2-(prop-2-yn-1-yloxy)ethoxy)ethyl)propanamide (66.4 mg, 99% yield). LC/MS: MH⁺=421.2, Rt=0.52 min (2 min acidic method).

### Synthesis of Methyl (2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate

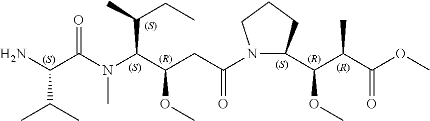

To the RB-flask contained Pd/C (10 wt %, Wet, Degussa type) (1.030 g, 10% Wt, 0.2 Eq, 968.1 μmol) was slowly added the solution of methyl (2R,3R)-3-((S)-1-((3R,4S,5S)-4-((S)-2-(((benzyloxy)carbonyl)amino)-N,3-dimethylbutanamido)-3-methoxy-5-methylheptanoyl)pyrrolidin-2-yl)-3-methoxy-2-methylpropanoate-methyl (2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate (3.000 g, 1 Eq, 4.840 mmol) in 54 ml of MeOH. The mixture was charged with H2 (1 atm) with a H2 balloon after three vacuum/H2 cycles, and stirred at room temperature overnight. The reaction mixture was filtered through Celite (pre-washed with DCM) through natural gravity and washed with DCM and then MeOH/DCM (1/1). The filtrate was concentrated in vacuo to give desired product as 2.337 g (99% yield) sticky oil to use for next reaction without further purification. HRMS: MH⁺=486.3611, Rt=1.52 min (5 min acidic method).

### Synthesis of Tert-butyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate

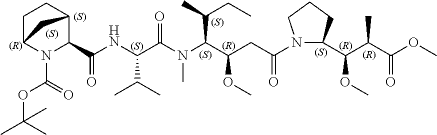

To the mixture of methyl (2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate (2.337 g, 1 Eq, 4.812 mmol), (1R,3S,4S)-2-(tert-butoxycarbonyl)-2-azabicyclo[2.2.1]heptane-3-carboxylic acid (1.277 g, 1.1 Eq, 5.293 mmol), and HATU (2.013 g, 1.1 Eq, 5.293 mmol) in DMF (20 ml) was added DIPEA (3.110 g, 4.19 mL, 5 Eq, 24.06 mmol).

The reaction mixture was stirred at RT for 3 h. The crude mixture was concentrated under vacuum to remove DIPEA and most DMF, then purified by C18 column (150 g cartridge, MeCN/Water with 0.05% TFA 0-100% over 18 CV) to obtain Tert-butyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate 2.704 g (79% yield). HRMS: MH⁺=709.4865, Rt=2.98 min (5 min acidic method).

### Synthesis of Methyl (2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoate

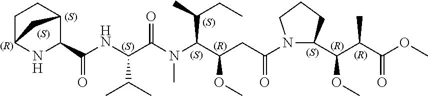

To the pre-cooled solution of tert-butyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (2.704 g, 1 Eq, 3.814 mmol) in DCM (22 ml) at 0° C. (ice-water bath) was added dropwise pre-cooled TFA (10.87 g, 7.346 mL, 25 Eq, 95.35 mmol). The mixture was stirred at 0° C. for 15 min, then raise to RT, and stirred at RT for 2 h. The crude was concentrated under high vacuum and then dried overnight under high vacuum, then was dissolved in MeOH, and purified by C-18 column (150 g cartridge, MeCN/Water with 0.05% TFA 0-55% over 18 CV) to obtain Methyl (2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoate (2.73 g, 99% yield). HRMS: MH⁺=609.4279, Rt=1.61 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate

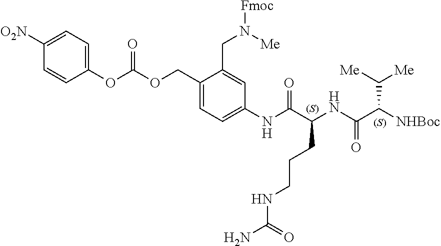

To the solution of (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (500.0 mg, 1 Eq, 671.2 μmol) in DMF (4 ml) was added bis(4-nitrophenyl) carbonate (408.4 mg, 2 Eq, 1.342 mmol), stir into fully soluble; and then followed by dropwise addition of DIPEA (260.3 mg, 357 μL, 3 Eq, 2.014 mmol). The reaction mixture was stirred at RT for 3 h. The crude was injected directly and purified by C-18 column (100 g cartridge, MeCN/Water 0-100% over 18 CV) to obtain (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (594 mg, 97% yield). HRMS: MH⁺=910.4000, Rt=3.05 min (5 min acidic method).

### Synthesis of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate

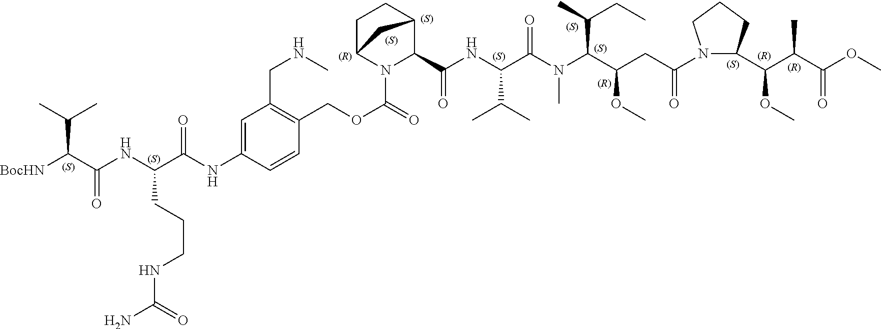

To a solution of Methyl (2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoate (440 mg, 1 Eq, 609 μmol) in DMF (4 ml) was added (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (609 mg, 1.10 Eq, 670 μmol), and then followed by DIPEA (472 mg, 636 μL, 6 Eq, 3.65 mmol). the reaction mixture was stirred at RT for 2 h. Then Me₂NH (2M in THF) (274 mg, 3.04 mL, 2 molar, 10 Eq, 6.09 mmol) was added, and stirred at RT for 30 min. The crude was concentrated under vacuum, then diluted with DMSO, followed by purification with C-18 column (100 g cartridge, MeCN/water with 0.05% TFA 0-65% over 18 CV) to obtain 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (303 mg, 40% yield). HRMS: MH⁺=1157.7408, Rt=2.10 min (5 min acidic method).

### Synthesis of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate

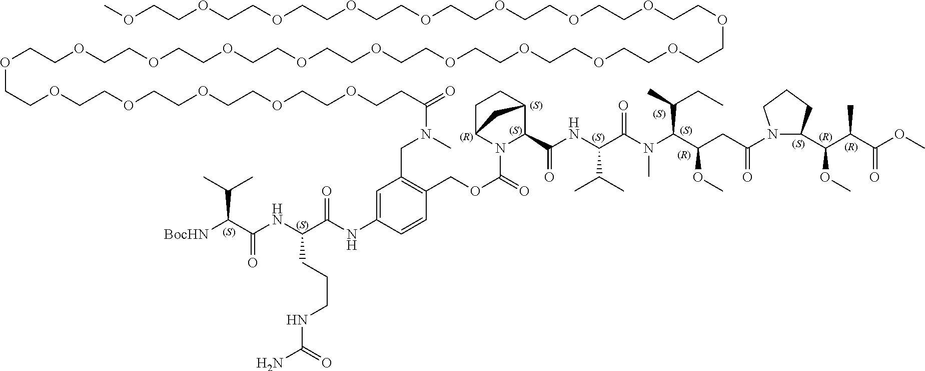

To 74-oxo-2,5,8,11,14,17,20,23,26,29,32,35,38,41,44,47,50,53,56,59,62,65,68,71-tetracosaoxapentaheptacontan-75-oic acid (399.4 mg, 1.5 Eq, 357.5 μmol) in DMF (1 ml) was added HATU (135.9 mg, 1.5 Eq, 357.5 μmol), and followed by DIPEA (184.8 mg, 249 μL, 6 Eq, 1.430 mmol). pH was tested as basic. The mixture was stirred for 30 min. To the above mixture, the solution of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (303.0 mg, 1 Eq, 238.3 μmol) in DMF (1 ml) was added. The mixture was stirred at Rt for 1 h. The crude was purified by C18 column (100 g cartridge, MeCN/Water with 0.05% TFA 0-70% over 18 CV) to obtain 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (387 mg, 72% yield). HRMS: MH⁺=2256.3501, Rt=2.63 min (5 min acidic method).

### Synthesis of (2R,3R)-3-((S)-1-((3R,4S,5S)-4-((S)-2-((1R,3S,4S)-2-(((4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid

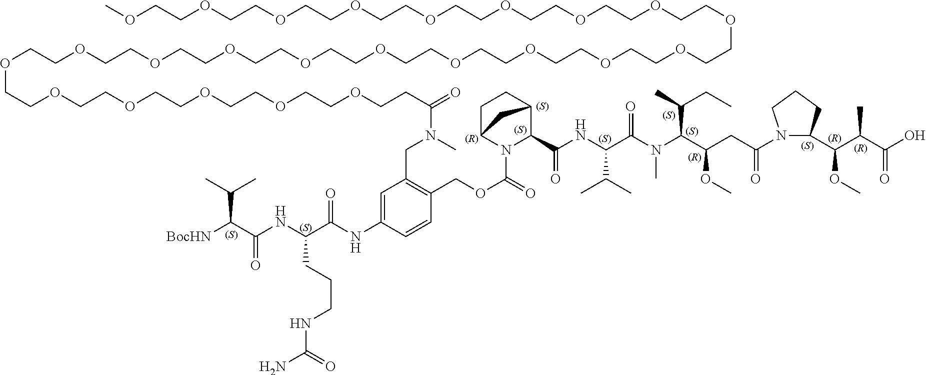

To the solution of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (1302.3 mg, 1 Eq, 577.06 μmol) in MeOH (8 ml), Water (8 ml) and THF (4 ml) was added LiOH (414.6 mg, 30 Eq, 17.312 mmol). The resulting reaction mixture was stirred at RT for 2 h. The crude mixture was added HCl (4M in dioxane, 3.8 ml) slowly to neutralize extra LiOH, but still keep the final solution pH as close to neutral, but not as acidic to avoid de-Boc. Then crude was purified with C18 column (275 g cartridge, MeCN/Water with 0.05% TFA 0-60% over 18 CV) to provide (2R,3R)-3-((S)-1-((3R,4S,5S)-4-((S)-2-((1R,3S,4S)-2-(((4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid (1.203 g, 92% yield). HRMS: MH⁺=2243.3601, Rt=2.49 min (5 min acidic method).

### Synthesis of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

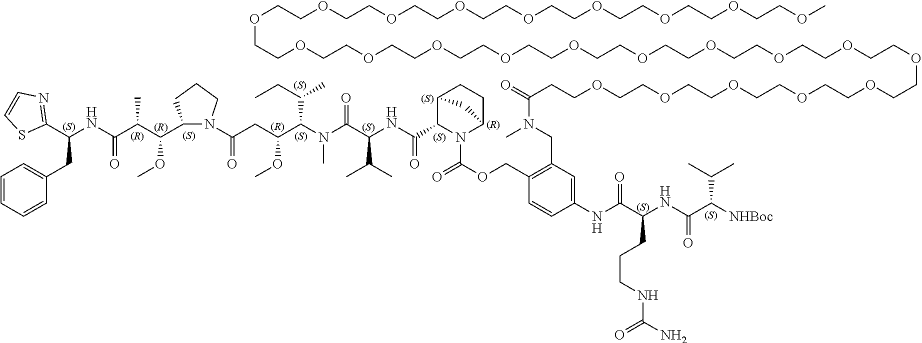

To the solution of (2R,3R)-3-((S)-1-((3R,4S,5S)-4-((S)-2-((1R,3S,4S)-2-(((4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid (644.0 mg, 1 Eq, 287.1 μmol) in DMF (6 ml) was added (S)-2-phenyl-1-(thiazol-2-yl)ethan-1-amine hydrochloride (89.87 mg, 1.3 Eq, 373.3 μmol), followed by HATU (141.9 mg, 1.3 Eq, 373.3 μmol), and DIPEA (222.7 mg, 300 μL, 6 Eq, 1.723 mmol). The reaction mixture turned into light brown, stir at RT for 2 h. The crude mixture was purified by C-18 column (150 g cartridge, MeCN/Water with 0.05% TFA 0-80% over 18 CV) to obtain 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (667 mg, 95% yield). HRMS: MH⁺=2429.4099, Rt=2.68 min (5 min acidic method).

### Synthesis of 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

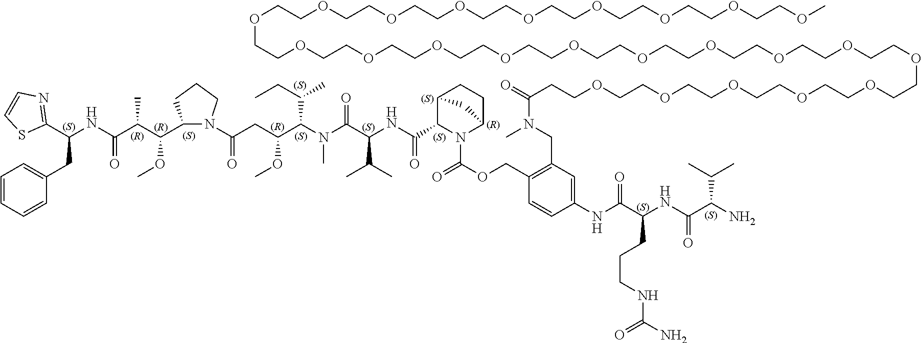

To the solution of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (667.2 mg, 1 Eq, 274.7 μmol) in DCM (1 ml) was added triethylsilane (319.4 mg, 439 μL, 10 Eq, 2.747 mmol). The mixture was cooled to 0° C. in ice-water bath, and then pre-cooled TFA (0.333 ml) (TFA/DCM, 25% v/v).

The mixture was stirred at 0° C. for 15 min, then raised to RT. The mixture was stirred at RT for 1 h.

The crude was purified by C18 column (100 g cartridge, MeCN/Water with 0.05% TFA 10-80% over 18 CV) to obtain 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (627 mg, 93% yield). HRMS: MH⁺=2328.3401, Rt=2.17 min (5 min acidic method).

### Synthesis of 4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

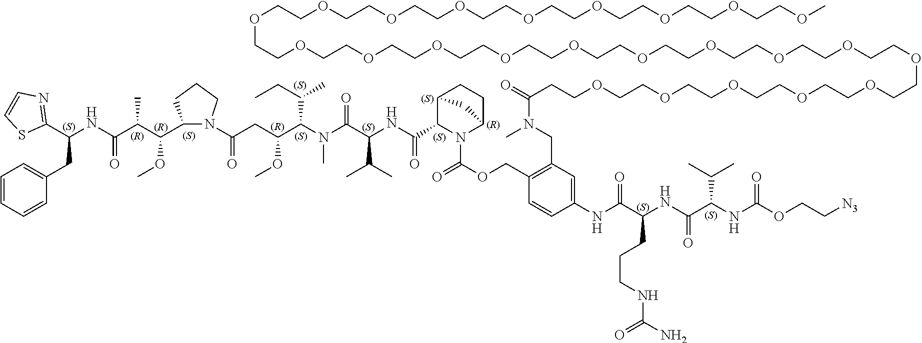

To 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (100 mg/ml in DMF) (214.7 mg, 2.15 mL, 1 Eq, 87.89 μmol) was added 2-azidoethyl (4-nitrophenyl) carbonate (100 mg/ml in DMF) (66.49 mg, 665 μL, 3 Eq, 263.7 μmol), and then DIPEA (90.87 mg, 122 μL, 8 Eq, 703.1 μmol). The pH was tested as basic. The mixture was stirred at RT for 1 h. The crude was purified by C18 column (100 g, MeCN/Water with 0.05% TFA 10-80% over 18 CV) to obtain 4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (179 mg, 83% yield). HRMS: MH⁺=2442.3799, Rt=2.59 min (5 min acidic method).

### Synthesis of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate

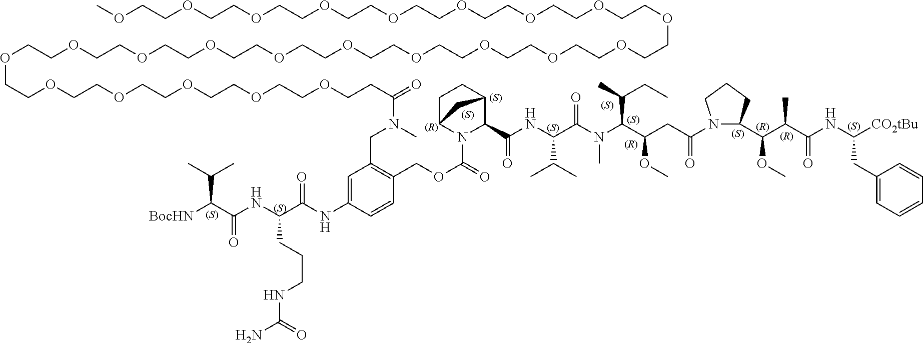

To (2R,3R)-3-((S)-1-((3R,4S,5S)-4-((S)-2-((1R,3S,4S)-2-(((4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid (100 mg/ml in DMF) (400.0 mg, 4.00 mL, 1 Eq, 178.4 μmol) was added tert-butyl L-phenylalaninate hydrochloride (59.76 mg, 1.3 Eq, 231.9 μmol), followed by HATU (88 mg, 1.3 Eq, 232 μmol), and DIPEA (138.3 mg, 186 μL, 6 Eq, 1.070 mmol). The reaction mixture turned into light brown, stir at RT for 2 h. The crude mixture was purified by C-18 column (150 g cartridge, MeCN/Water with 0.05% TFA 0-80% over 18 CV) to obtain 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (383 mg, 88% yield). HRMS: MH⁺=2445.4900, Rt=2.92 min (5 min acidic method).

### Synthesis of ((2R,3R)-3-((S)-1-((3R,4S,5S)-4-((S)-2-((1R,3S,4S)-2-(((4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine

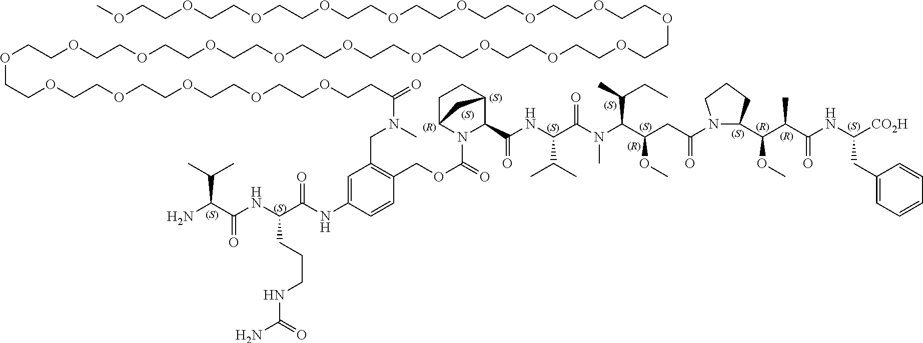

To the solution of 4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (347.5 mg, 1 Eq, 142.1 μmol) in DCM (1.2 ml) was added triethylsilane (165.2 mg, 227 μL, 10 Eq, 1.421 mmol). The mixture was cooled to OC in ice-water bath, and then pre-cooled TFA (0.4 ml) (TFA/DCM, 25% v/v). The mixture was stirred at 0° C. for 15 min, then raised to RT. The mixture was stirred at RT for 1 h. The crude was concentrated under high vacuum, then purified by C18 Column (100 g cartridge, MeCN/Water with 0.05% TFA 0-80% over 18 CV) to obtain ((2R,3R)-3-((S)-1-((3R,4S,5S)-4-((S)-2-((1R,3S,4S)-2-(((4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine (330 mg, 96% yield). HRMS: MH⁺=2290.3799, Rt=2.09 min (5 min acidic method).

### Synthesis of ((((2S,5S,39S,42S)-22-(3-(2-((tert-butoxycarbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene) (1R,1′R,3S,3'S,4S,4'S)-bis(3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate)

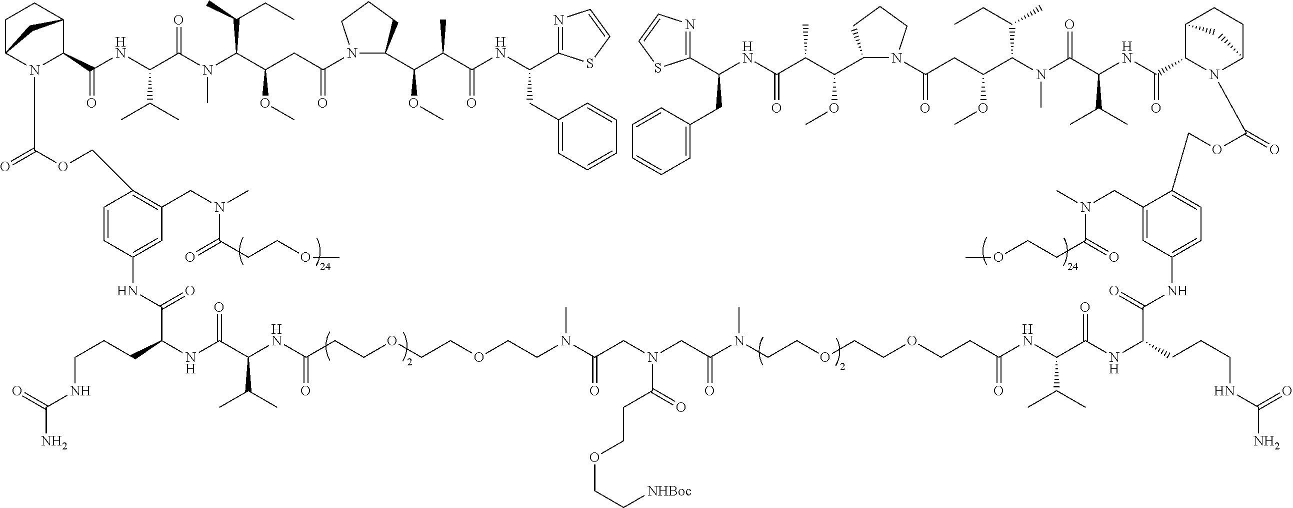

To 16-(3-(2-((tert-butoxycarbonyl)amino)ethoxy)propanoyl)-13,19-dimethyl-14,18-dioxo-4,7,10,22,25,28-hexaoxa-13,16,19-triazahentriacontanedioic acid (25 mg/ml in DMF) (10.00 mg, 0.40 mL, 1 Eq, 12.77 μmol) was added HATU (100 mg/ml) (11.66 mg, 117 μL, 2.4 Eq, 30.66 μmol) and then DIPEA (19.81 mg, 26.7 μL, 12 Eq, 153.3 μmol). The mixture was stirred at RT for 30 min. Then to the above mixture, 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (100 mg/ml in DMF)(68.65 mg, 686 μL, 2.2 Eq, 28.10 μmol) was added. The mixture was stirred at RT for 90 min. The crude mixture was purified with C4 column (50 g cartridge, MeCN/Water with 0.05% TFA 0-80% over 18 CV) to obtain ((((2S,5S,39S,42S)-22-(3-(2-((tert-butoxycarbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene) (1R,1′R,3S,3'S,4S,4'S)-bis(3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate) (32 mg, 47% yield). HRMS: MH⁺=5402.1401, Rt=2.87 min (5 min acidic method).

### Example #1: Synthesis of {4-[(2S,5S,39S,42S)-47-Amino-2-(3-carbamamidopropyl)-22-[1-(2,5-dioxo-2,5-dihydro-1H-pyrrol-1-yl)-15,22-dioxo-3,6,9,12,19-pentaoxa-16-azadocosan-22-yl]-42-{[4-({[(1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-19,25-dimethyl-4,7,20,24,37,40,47-heptaoxo-5,39-di(propan-2-yl)-10,13,16,28,31,34-hexaoxa-3,6,19,22,25,38,41,46-octaazaheptatetracontanan-1-amido]-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl}methyl (1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carboxylate

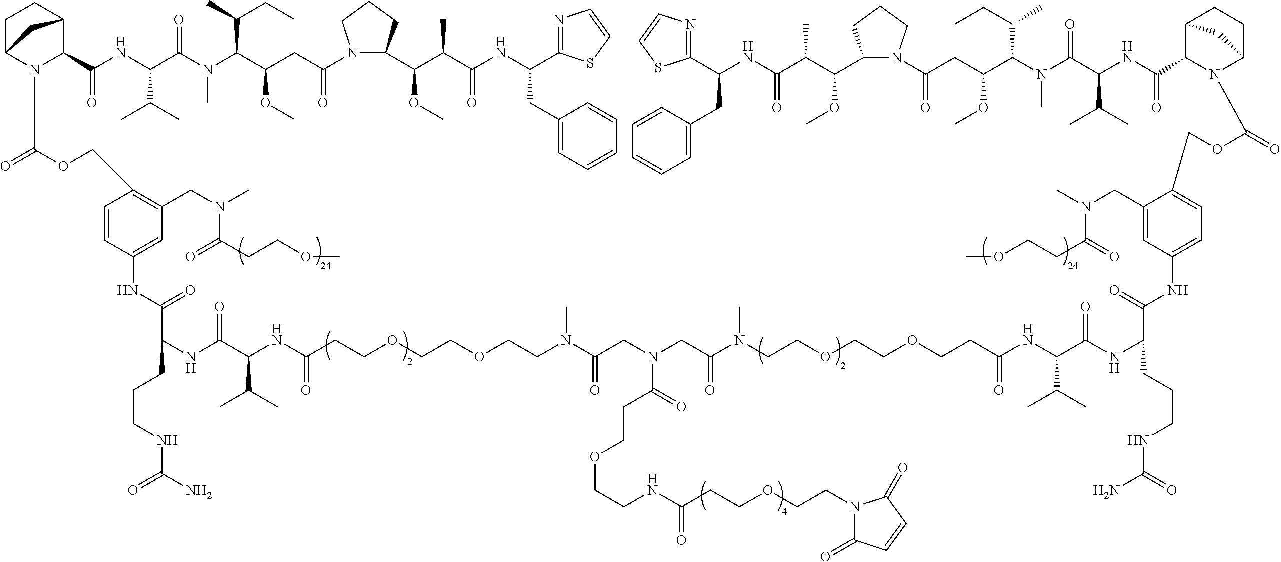

To the pre-cooled solution of ((((2S,5S,39S,42S)-22-(3-(2-((tert-butoxycarbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene) (1R,1′R,3S,3'S,4S,4'S)-bis(3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (60.60 mg, 1 Eq, 11.21 μmol) in DCM (1.5 ml) at 0° C. (ice-water bath) was added pre-cooled TFA (0.5 ml) (TFA/DCM as 25% v/v). The mixture was stirred at 0° C. for 15 min, then raised to RT and stir at RT for 30 min. The mixture was then concentrated under high vacuum for 1 h. To the resulting mixture in DMF (1 ml) was added 2,5-dioxopyrrolidin-1-yl 1-(2,5-dioxo-2,5-dihydro-1H-pyrrol-1-yl)-3,6,9,12-tetraoxapentadecan-15-oate (14.88 mg, 3 Eq, 33.64 μmol), and followed by DIPEA (21.74 mg, 29.3 μL, 15 Eq, 168.2 μmol) The reaction mixture was stirred at RT for 30 min. The crude mixture separated with C4 column (not C-18) (50 g cartridge, MeCN/Water with 0.05% TFA, 0-80% over 18 CV) to obtain {4-[(2S,5S,39S,42S)-47-Amino-2-(3-carbamamidopropyl)-22-[1-(2,5-dioxo-2,5-dihydro-1H-pyrrol-1-yl)-15,22-dioxo-3,6,9,12,19-pentaoxa-16-azadocosan-22-yl]-42-{[4-({[(1R,3S,4S)-3-{[(2S)-1-{[(3R,4S,5S)-3-methoxy-1-{(2S)-2-[(1R,21R)-1-methoxy-2-methyl-3-oxo-3-{[(1 S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-19,25-dimethyl-4,7,20,24,37,40,47-heptaoxo-5,39-di(propan-2-yl)-10,13,16,28,31,34-hexaoxa-3,6,19,22,25,38,41,46-octaazaheptatetracontanan-1-amido]-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl}methyl (1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carboxylate (30 mg, 47% yield). HRMS: MH⁺=5629.2300, Rt=2.80 min (5 min acidic method).

### Synthesis of {4-[(2S)-2-{(2S)-2-[({2-[4-(8-{2-[2-({1-[(6S,9S)-14-Amino-9-{[4-({[(1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-18,18-dimethyl-9,16-dioxo-2,5,12,17-tetraoxa-8,15-diazanonadecan-1-yl)-1H-1,2,3-triazol-1-yl]ethoxy}carbonyl)amino]-3-methylbutanamido}-5-carbamamidopentanamido]-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl}methyl (1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carboxylate

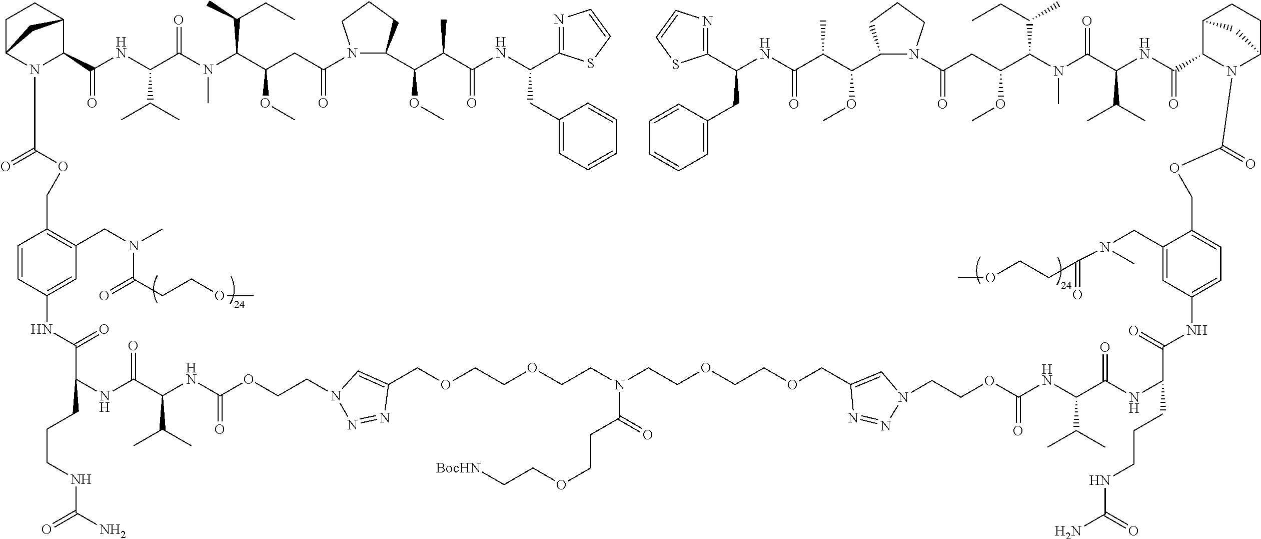

A mixture of Tert-butyl (6-oxo-7-(2-(2-(prop-2-yn-1-yloxy)ethoxy)ethyl)-3,10,13-trioxa-7-azahexadec-15-yn-1-yl)carbamate (50 mg/ml in DMSO) (5.0 mg, 0.100 mL, 1 Eq, 10 μmol) and 4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (50 mg/ml in DMSO) (60 mg, 1.2 mL, 2.4 Eq, 25 μmol) was vacuumed and purged with N2, repeat three times. Sodium Ascorbate (32 mg/ml in water) (7.4 mg, 0.23 mL, 3.6 Eq, 37 μmol), and then CuSO4·5H₂O (16 mg/ml in water) (3.1 mg, 0.19 mL, 1.2 Eq, 12 μmol) was added. The resulting mixture was vacuumed and purged with N2, repeat 3 times. Then the mixture was stirred under N2 atmosphere at RT for 90 min. The reaction mixture was directly uploaded and separated by C4 column (50 g cartridge, MeCN/Water with 0.05% TFA 0-90% over 16 CV) to obtain {4-[(2S)-2-{(2S)-2-[({2-[4-(8-{2-[2-({1-[(6S,9S)-14-Amino-9-{[4-({[(1R,3S,4S)-3-{[(2S)-1-{[(3R,4S,5S)-3- methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-18,18-dimethyl-9,16-dioxo-2,5,12,17-tetraoxa-8,15-diazanonadecan-1-yl)-1H-1,2,3-triazol-1-yl]ethoxy}carbonyl)amino]-3-methylbutanamido}-5-carbamamidopentanamido]-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl}methyl (1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carboxylate (49 mg, 89% yield). HRMS: MH⁺=5365.9902, Rt=2.87 min (5 min acidic method).

### Example #2: Synthesis of ((((2S,2′S)-2,2′-(((2S,2′S)-2,2′-((((((8-(1-(2,5-dioxo-2,5-dihydro-1H-pyrrol-1-yl)-15-oxo-3,6,9,12,19-pentaoxa-16-azadocosan-22-oyl)-2,5,11,14-tetraoxa-8-azapentadecane-1,15-diyl)bis(1H-1,2,3-triazole-4,1-diyl))bis(ethane-2,1-diyl))bis(oxy))bis(formyl))bis(azanediyl))bis(3-methylbutanoyl))bis(azanediyl))bis(5-ureidopentanoyl))bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene) (1R,1′R,3S,3'S,4S,4'S)-bis(3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate)

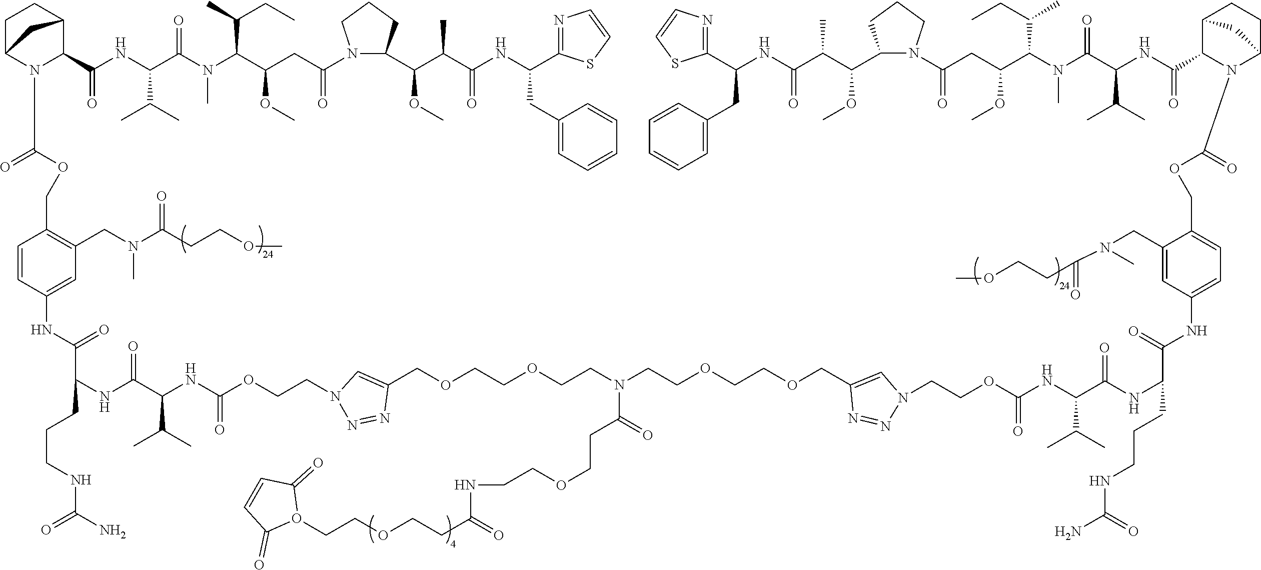

To the pre-cooled solution of {4-[(2S)-2-{(2S)-2-[({2-[4-(8-{2-[2-({1-[(6S,9S)-14-Amino-9-{[4-({[(1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-18,18-dimethyl-9,16-dioxo-2,5,12,17-tetraoxa-8,15-diazanonadecan-1-yl)-1H-1,2,3-triazol-1-yl]ethoxy}carbonyl)amino]-3-methylbutanamido}-5-carbamamidopentanamido]-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl}methyl (1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carboxylate (49.1 mg, 1 Eq, 9.15 μmol) in DCM (1.5 ml) at 0° C. (ice-water bath) was added pre-cooled TFA (0.5 ml) (TFA/DCM as 25% v/v). The mixture was stirred at 0° C. for 15 min, then raised to RT and stir at RT for 30 min. The mixture was concentrated under high vacuum for 1 h. To the above mixture and 2,5-dioxopyrrolidin-1-yl 1-(2,5-dioxo-2,5-dihydro-1H-pyrrol-1-yl)-3,6,9,12-tetraoxapentadecan-15-oate (12.1 mg, 3 Eq, 27.4 μmol) in DMF (1 ml) was added DIPEA (17.7 mg, 23.9 μL, 15 Eq, 137 μmol). The reaction mixture was stirred at RT for 30 min. The mixture was separated with C4 column (50 g cartridge, MeCN/Water with 0.05% TFA, 0-80% over 18 CV) to obtain ((((2S,2′S)-2,2′-(((2S,2′S)-2,2′-((((((8-(1-(2,5-dioxo-2,5-dihydro-1H-pyrrol-1-yl)-15-oxo-3,6,9,12,19-pentaoxa-16-azadocosan-22-oyl)-2,5,11,14-tetraoxa-8-azapentadecane-1,15-diyl)bis(1H-1,2,3-triazole-4,1-diyl))bis(ethane-2,1-diyl))bis(oxy))bis(formyl))bis(azanediyl))bis(3-methylbutanoyl))bis(azanediyl))bis(5-ureidopentanoyl))bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene) (1R,1′R,3S,3'S,4S,4'S)-bis(3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate) (28 mg, 54% yield). HRMS: MH⁺=5593.1499, Rt=2.80 min (5 min acidic method).

### Synthesis of 4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

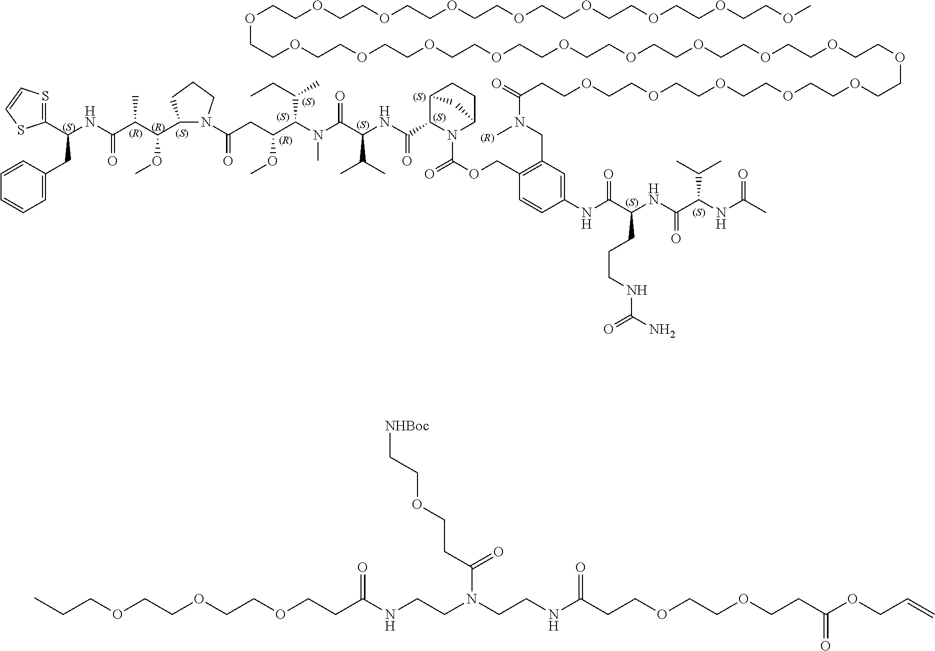

17-(3-(2-((tert-butoxycarbonyl)amino)ethoxy)propanoyl)-13,21,30-trioxo-4,7,10,24,27,31-hexaoxa-14,17,20-triazatetratriacont-33-enoic acid (25 mg/ml in DMF) (20.00 mg, 0.80 mL, 1 Eq, 25.68 μmol) was added HATU (100 mg/ml in DMF) (11.72 mg, 117 μL, 1.2 Eq, 30.81 μmol), and then DIPEA (19.91 mg, 26.8 μL, 6 Eq, 154.1 μmol). The reaction mixture was stirred for 30 min. Then 4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (100 mg/ml in DMF)(75.27 mg, 753 μL, 1.2 Eq, 30.81 μmol) was then added. The mixture was stirred at RT for 1 h. The crude was purified by C18 column (100 cartridge, MeCN/Water with 0.05% TFA 0-80% over 18 CV) to obtain 4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (58 mg, 73% yield). HRMS: MH⁺=3088.7500, Rt=2.64 min (5 min acidic method).

### Synthesis of (6S,9S)-1-amino-27-(3-(2-((tert-butoxycarbonyl)amino)ethoxy)propanoyl)-9-isopropyl-6-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-1,8,11,23,31-pentaoxo-14,17,20,34,37-pentaoxa-2,7,10,24,27,30-hexaazatetracontan-40-oic acid

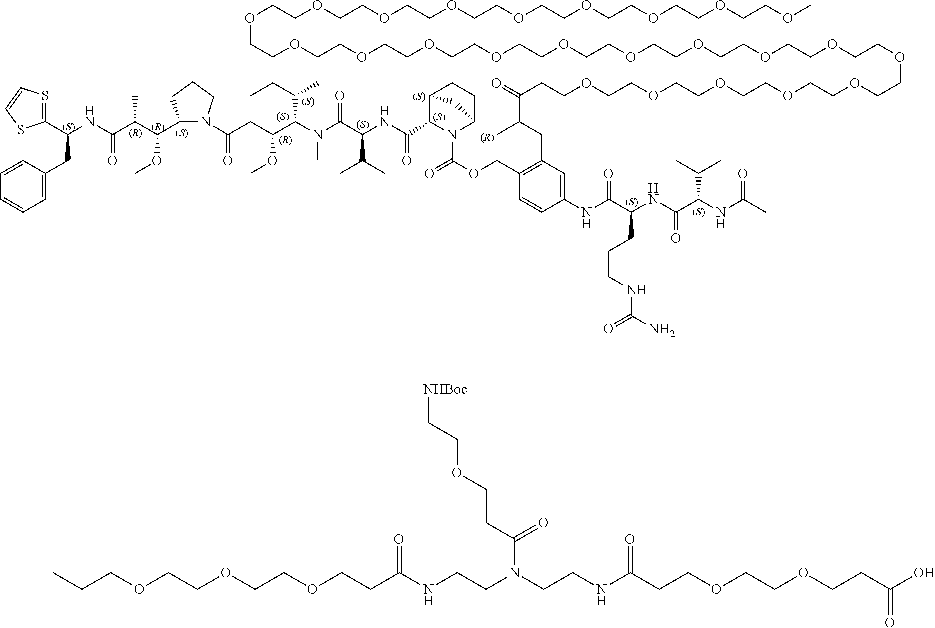

To the solution of 4-((2S,5S)-23-(3-(2-((tert-butoxycarbonyl)amino)ethoxy)propanoyl)-5-isopropyl-4,7,19,27,36-pentaoxo-2-(3-ureidopropyl)-10,13,16,30,33,37-hexaoxa-3,6,20,23,26-pentaazatetracont-39-enamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (110.9 mg, 1 Eq, 35.89 μmol) in THF/MeOH (1 ml/1 ml, total 2 ml) was added phenylsilane (7.769 mg, 8.85 μL, 2 Eq, 71.79 μmol). The solution was degassed under vacuum and then filled with N2 through N2 balloon; repeated 3×; then Pd(Ph₃P)₄ (414.8 μg, 0.01 Eq, 0.3589 μmol) was added to the solution. The reaction mixture was degassed under vacuum and then filled with N2 through N2 balloon; repeated 3×. The reaction mixture was stirred at RT under N2 overnight. The crude was filtered through celite filter, then washed with MeOH, concentrated under vacuum to remove volatiles, the resulting crude was dissolved in DMSO, and purified by C18 column (100 g cartridge, MeCN/water with 0.05% TFA 0-55% over 18 CV) to obtain (6S,9S)-1-amino-27-(3-(2-((tert-butoxycarbonyl)amino)ethoxy)propanoyl)-9-isopropyl-6-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-1,8,11,23,31-pentaoxo-14,17,20,34,37-pentaoxa-2,7,10,24,27,30-hexaazatetracontan-40-oic acid (67 mg, 61% yield). HRMS: MH⁺=3048.7231, Rt=2.50 min (5 min acidic method).

### Synthesis of ((2R,3R)-3-((S)-1-((3R,4S,5S)-4-((S)-2-((1R,3S,4S)-2-(((4-((S)-2-((S)-2-((S)-14-(3-(2-((tert-butoxycarbonyl)amino)ethoxy)propanoyl)-32-(((S)-1-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)carbamoyl)-33-methyl-10,18,30-trioxo-4,7,21,24,27-pentaoxa-11,14,17,31-tetraazatetratriacontanamido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine

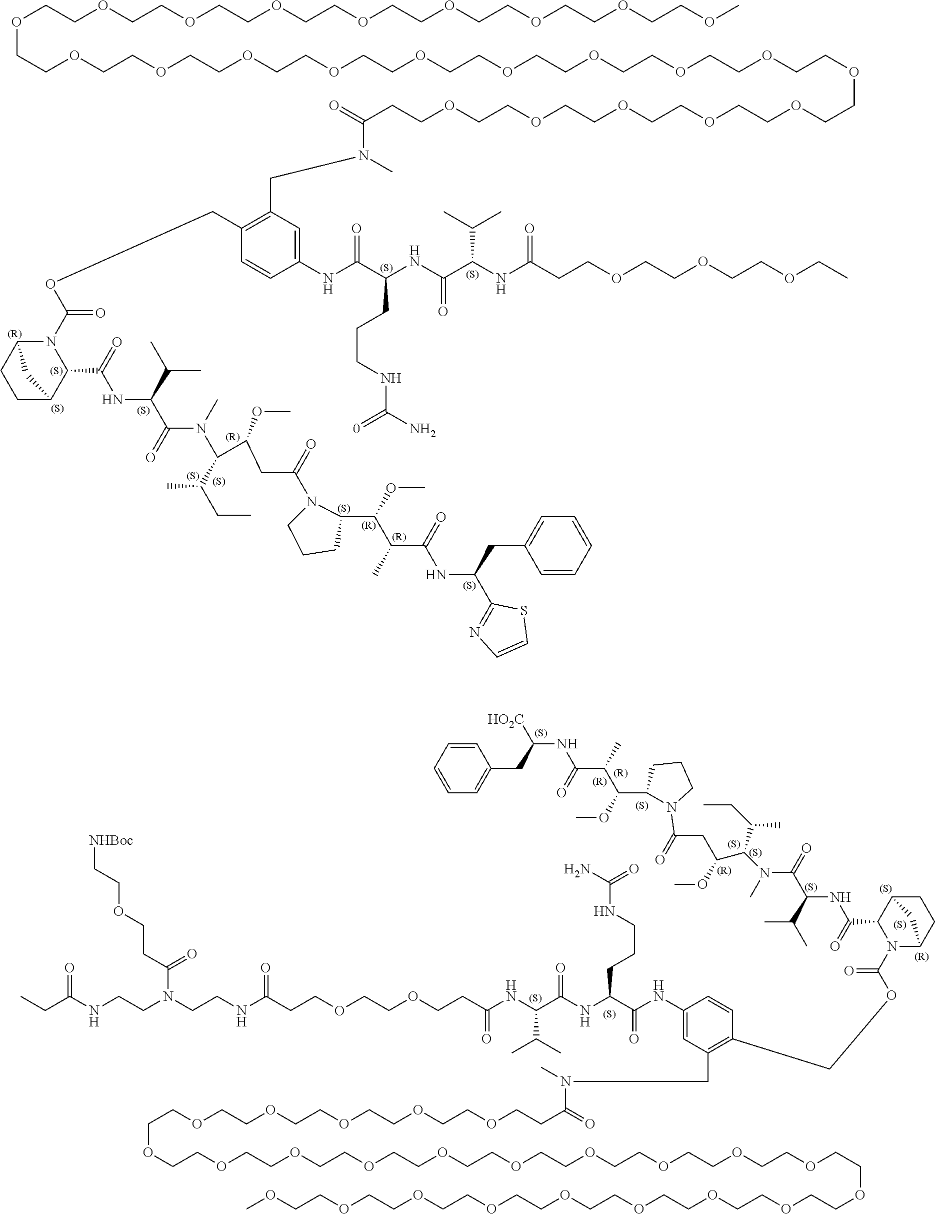

(6S,9S)-1-amino-27-(3-(2-((tert-butoxycarbonyl)amino)ethoxy)propanoyl)-9-isopropyl-6-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-1,8,11,23,31-pentaoxo-14,17,20,34,37-pentaoxa-2,7,10,24,27,30-hexaazatetracontan-40-oic acid (67 mg, 22 μmol) was added TSTU (50 mg/ml in NMP) (6.976 mg, 0.14 mL, 1.05 Eq, 23.17 μmol), and then DIPEA (22.82 mg, 30.8 μL, 8 Eq, 176.5 μmol). The mixture was stirred for 30 min. To the above mixture, ((2R,3R)-3-((S)-1-((3R,4S,5S)-4-((S)-2-((1R,3S,4S)-2-(((4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine (80 mg/ml in NMP) (95.48 mg, 1.2 mL, 1.8 Eq, 39.72 μmol) was added, then extra DIPEA (22.82 mg, 30.8 μL, 8 Eq, 176.5 μmol) was added. The mixture was stirred for 1 h. The crude was purified with C4 column (50 g cartridge, MeCN/water with 0.05% TFA 0-90% over 18 CV) to obtain ((2R,3R)-3-((S)-1-((3R,4S,5S)-4-((S)-2-((1R,3S,4S)-2-(((4-((S)-2-((S)-2-((S)-14-(3-(2-((tert-butoxycarbonyl)amino)ethoxy)propanoyl)-32-(((S)-1-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)carbamoyl)-33-methyl-10,18,30-trioxo-4,7,21,24,27-pentaoxa-11,14,17,31-tetraazatetratriacontanamido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine (76 mg, 65% yield). HRMS: MH⁺=5319.0498, Rt=2.76 min (5 min acidic method).

### Example #3: Synthesis of ((2R,3R)-3-((S)-1-((3R,4S,5S)-4-((S)-2-((1R,3S,4S)-2-(((4-((S)-2-((S)-2-(1-(2,5-dioxo-2,5-dihydro-1H-pyrrol-1-yl)-23-((S)-18-(((S)-1-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)carbamoyl)-19-methyl-4,16-dioxo-7,10,13-trioxa-3,17-diazaicosyl)-15,22,27-trioxo-3,6,9,12,19,30,33-heptaoxa-16,23,26-triazahexatriacontan-36-amido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine

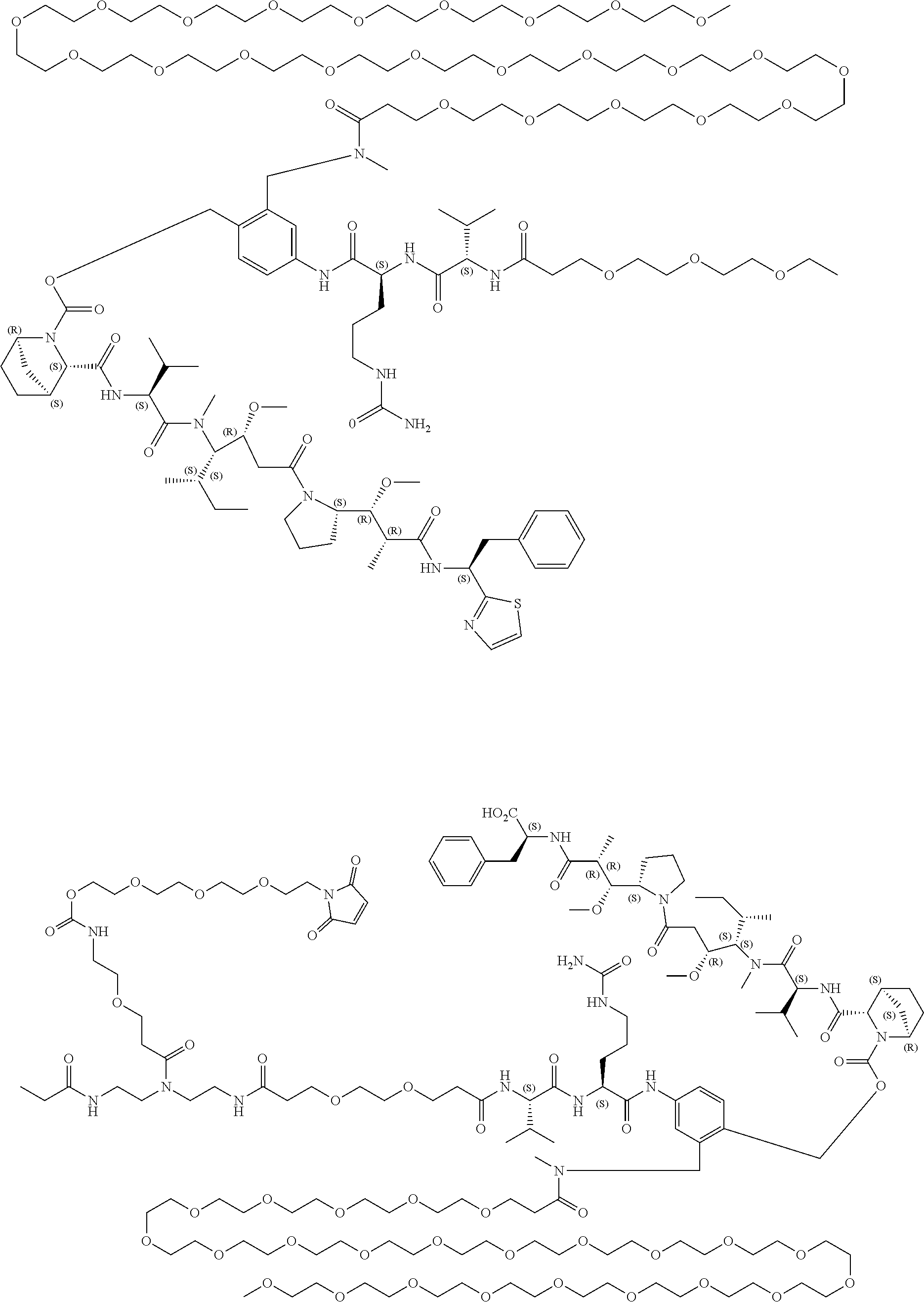

To the pre-cooled solution of ((2R,3R)-3-((S)-1-((3R,4S,5S)-4-((S)-2-((1R,3S,4S)-2-(((4-((S)-2-((S)-2-((S)-14-(3-(2-((tert-butoxycarbonyl)amino)ethoxy)propanoyl)-32-(((S)-1-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)carbamoyl)-33-methyl-10,18,30-trioxo-4,7,21,24,27-pentaoxa-11,14,17,31-tetraazatetratriacontanamido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine (76.50 mg, 1 Eq, 14.38 μmol) in DCM (1.5 ml) at 0° C. (ice-water bath) was added pre-cooled TFA (0.5 ml) (TFA/DCM as 25% v/v). The mixture was stirred at 0° C. for 15 min, then raised to RT and stir at Rt for 30 min. The mixture was concentrated under high vacuum for 1 h. To the mixture of the above crude and 2,5-dioxopyrrolidin-1-yl 1-(2,5-dioxo-2,5-dihydro-1H-pyrrol-1-yl)-3,6,9,12-tetraoxapentadecan-15-oate (19.08 mg, 3 Eq, 43.13 μmol) in DMF (1 ml)) was added DIPEA (27.87 mg, 37.6 μL, 15 Eq, 215.6 μmol) The reaction mixture was stirred at RT for 30 min. The mixture was separated with C4 column (50 g cartridge, MeCN/Water with 0.05% TFA, 0-80% over 18 CV) to obtain ((2R,3R)-3-((S)-1-((3R,4S,5S)-4-((S)-2-((1R,3S,4S)-2-(((4-((S)-2-((S)-2-(1-(2,5-dioxo-2,5-dihydro-1H-pyrrol-1-yl)-23-((S)-18-(((S)-1-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)amino)-1-oxo-5-ureidopentan-2-yl)carbamoyl)-19-methyl-4,16-dioxo-7,10,13-trioxa-3,17-diazaicosyl)-15,22,27-trioxo-3,6,9,12,19,30,33-heptaoxa-16,23,26-triazahexatriacontan-36-amido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine (38 mg, 48% yield). HRMS: MH⁺=5546.1299, Rt=2.71 min (5 min acidic method).

### Synthesis of 12-(2-(2-((1-((6S,9S)-1-amino-9-isopropyl-6-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-1,8,11-trioxo-12-oxa-2,7,10-triazatetradecan-14-yl)-1H-1,2,3-triazol-4-yl)methoxy)ethoxy)ethyl)-2,2-dimethyl-4,11-dioxo-3,8,15,18,21-pentaoxa-5,12-diazatetracosan-24-oic acid

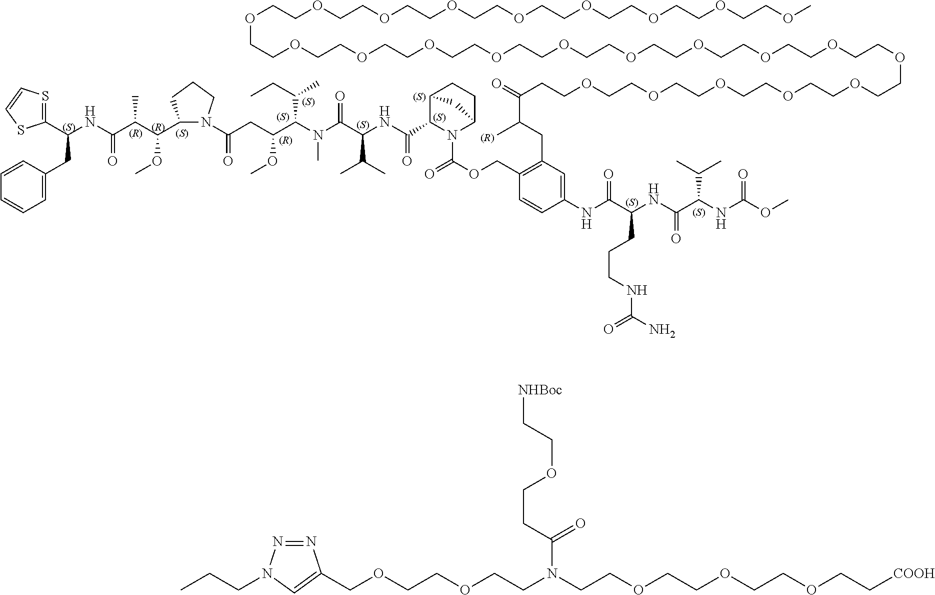

A mixture of 4,7,13,16,19-pentaoxa-10-azadocos-1-yn-22-oic acid, 2,2-dimethyl-4,11-dioxo-12-(2-(2-(prop-2-yn-1-yloxy)ethoxy)ethyl)-3,8,15,18,21-pentaoxa-5,12-diazatetracosan-24-oic acid (25 mg/ml in DMSO) (12.56 mg, 0.50 mL, 1 Eq, 22.32 μmol) and 4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (50 mg/ml in DMSO) (59.96 mg, 1.2 mL, 1.1 Eq, 24.55 μmol) was vacuumed and purged with N2, repeat three times. Then Sodium Ascorbate (32 mg/ml in water) (15.92 mg, 0.50 mL, 3.6 Eq, 80.36 μmol) and followed by CuSO₄.5H₂O (16 mg/ml in water) (6.688 mg, 0.42 mL, 1.2 Eq, 26.79 μmol) was added. The resulting mixture was vacuumed and purged with N2, repeat 3 times. Then the mixture was stirred under N2 atmosphere at RT for 90 min. The reaction mixture was directly uploaded and separated by C-18 column (50 g C-4, MeCN/Water with 0.05% TFA 0-90% over 16 CV) to obtain 12-(2-(2-((1-((6S,9S)-1-amino-9-isopropyl-6-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-1,8,11-trioxo-12-oxa-2,7,10-triazatetradecan-14-yl)-1H-1,2,3-triazol-4-yl)methoxy)ethoxy)ethyl)-2,2-dimethyl-4,11-dioxo-3,8,15,18,21-pentaoxa-5,12-diazatetracosan-24-oic acid (63 mg, 95% yield). HRMS: MH⁺=3033.7141, Rt=2.59 min (5 min acidic method).

### Synthesis of (2S)-2-{(2R,3R)-3-[(2S)-1-{(3R,4S,5S)-4-[(2S)-2-{[(1R,3S,4S)-2-({[4-{[(26S,29S)-12-{2-[2-({1-[(6S,9S)-14-Amino-9-{[4-({[(1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-29-(3-carbamamidopropyl)-2,2-dimethyl-4,11,24,27,30-pentaoxo-26-(propan-2-yl)-3,8,15,18,21-pentaoxa-5,12,25,28-tetraazatriacontan-30-yl]amino}-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]methoxy}carbonyl)-2-azabicyclo[2.2.1]heptane-3-carbonyl]amino}-N,3-dimethylbutanamido]-3-methoxy-5-methylheptanoyl}pyrrolidin-2-yl]-3-methoxy-2-methylpropanamido}-3-phenylpropanoic acid

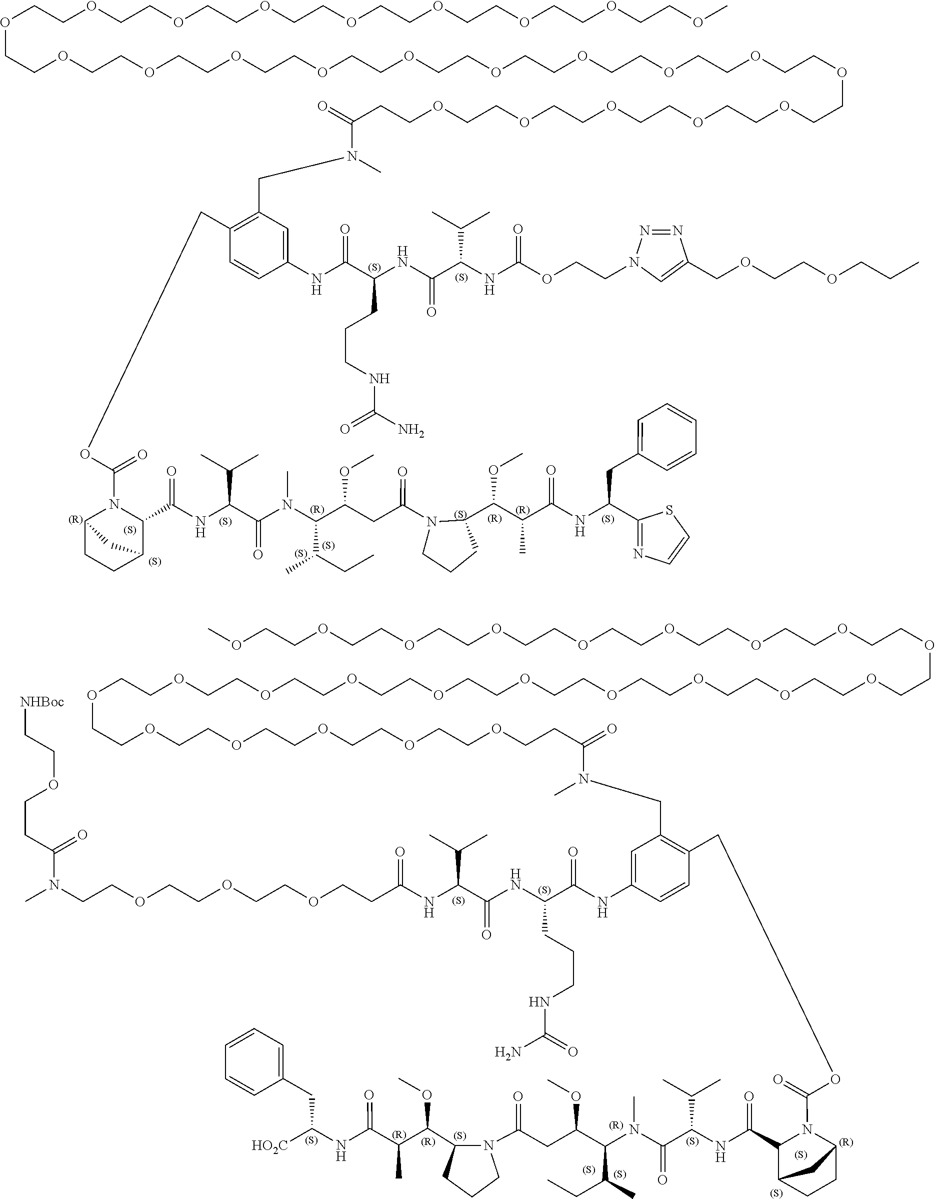

12-(2-(2-((1-((6S,9S)-1-amino-9-isopropyl-6-((4-((((1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carbonyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-1,8,11-trioxo-12-oxa-2,7,10-triazatetradecan-14-yl)-1H-1,2,3-triazol-4-yl)methoxy)ethoxy)ethyl)-2,2-dimethyl-4,11-dioxo-3,8,15,18,21-pentaoxa-5,12-diazatetracosan-24-oic acid (30 mg/ml in NMP) (64 mg, 2.133 mL, 1 Eq, 21 μmol) was added TSTU (50 mg/ml in NMP) (6.7 mg, 0.13 mL, 1.05 Eq, 22 μmol), and then DIPEA (22 mg, 30 μL, 8 Eq, 0.17 mmol). the mixture was stirred for 30 min. To the above mixture, ((2R,3R)-3-((S)-1-((3R,4S,5S)-4-((S)-2-((1R,3S,4S)-2-(((4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine (80 mg/ml in NMP)(92 mg, 1.2 mL, 1.8 Eq, 38 μmol) was added, extra DIPEA (22 mg, 30 μL, 8 Eq, 0.17 mmol) was added. The mixture was stirred at RT for 2 h. The crude was purified with C4 column (50 g cartridge, MeCN/water with 0.05% TFA 0-90% over 18 CV) to obtain (2S)-2-{(2R,3R)-3-[(2S)-1-{(3R,4S,5S)-4-[(2S)-2-{[(1R,3S,4S)-2-({[4-{[(26S,29S)-12-{2-[2-({1-[(6S,9S)-14-Amino-9-{[4-({[(1R,3S,4S)-3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-29-(3-carbamamidopropyl)-2,2-dimethyl-4,11,24,27,30-pentaoxo-26-(propan-2-yl)-3,8,15,18,21-pentaoxa-5,12,25,28-tetraazatriacontan-30-yl]amino}-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]methoxy}carbonyl)-2-azabicyclo[2.2.1]heptane-3-carbonyl]amino}-N,3-dimethylbutanamido]-3-methoxy-5-methylheptanoyl}pyrrolidin-2-yl]-3-methoxy-2-methylpropanamido}-3-phenylpropanoic acid (58.6 mg, 52% yield). HRMS: MH⁺=5274.0098, Rt=2.82 min (5 min acidic method).

### Example #4: Synthesis of (2S)-2-{(2R,3R)-3-[(2S)-1-{(3R,4S,5S)-4-[(2S)-2-{[(1R,3S,4S)-2-({[4-{[(37S,40S)-23-{2-[2-({1-[(6S,9S)-14-Amino-9-{[4-({[(1R,3S,4S)- 3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-40-(3-carbamamidopropyl)-1-(2,5-dioxo-2,5-dihydro-1H-pyrrol-1-yl)-15,22,35,38,41-pentaoxo-37-(propan-2-yl)-3,6,9,12,19,26,29,32-octaoxa-16,23,36,39-tetraazahentetracontan-41-yl]amino}-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]methoxy}carbonyl)-2-azabicyclo[2.2.1]heptane-3-carbonyl]amino}-N,3-dimethylbutanamido]-3-methoxy-5-methylheptanoyl}pyrrolidin-2-yl]-3-methoxy-2-methylpropanamido}-3-phenylpropanoic acid

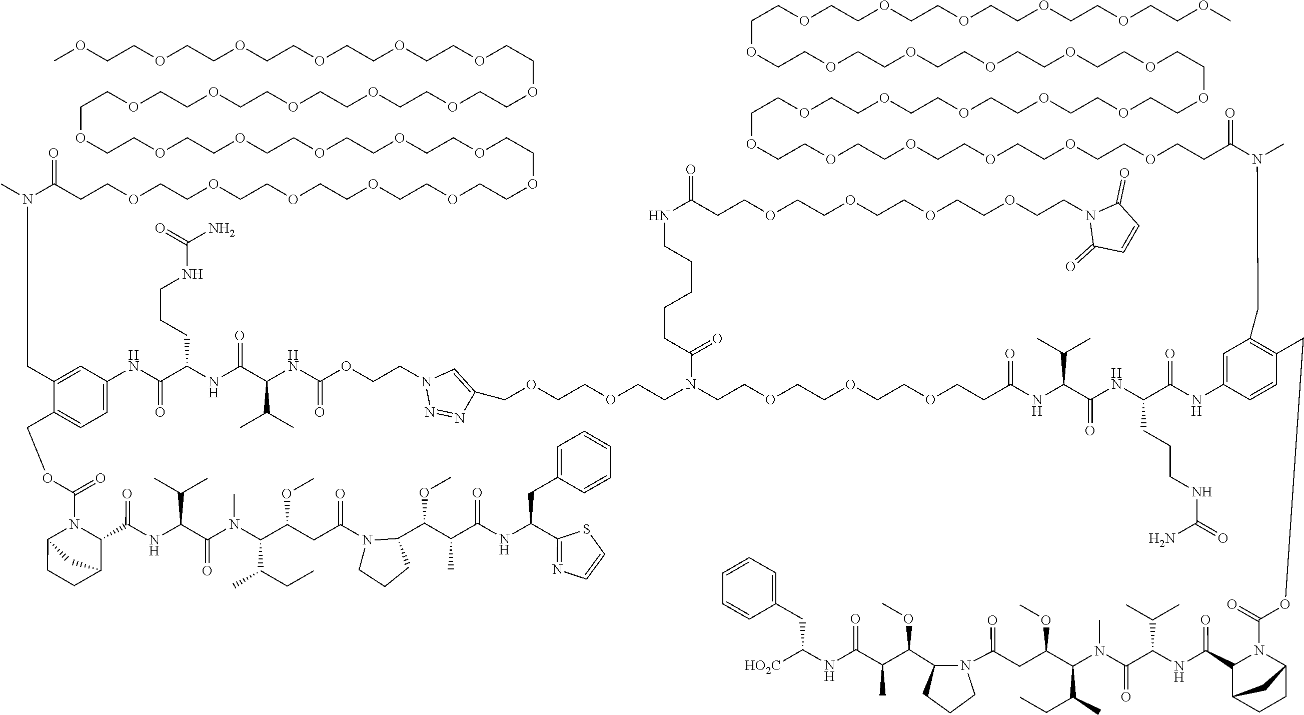

To the pre-cooled solution of (2S)-2-{(2R,3R)-3-[(2S)-1-{(3R,4S,5S)-4-[(2S)-2-{[(1R,3S,4S)-2-({[4-{[(26S,29S)-12-{2-[2-({1-[(6S,9S)-14-Amino-9-{[4-({[(1R,3S,4S)- 3-{[(2S)-1-{[(3R,4S,5S)-3-methoxy-1-{(2S)-2-[(1R,2R)-1-methoxy-2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-29-(3-carbamamidopropyl)-2,2-dimethyl-4,11,24,27,30-pentaoxo-26-(propan-2-yl)-3,8,15,18,21-pentaoxa-5,12,25,28-tetraazatriacontan-30-yl]amino}-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]methoxy}carbonyl)-2-azabicyclo[2.2.1]heptane-3-carbonyl]amino}-N,3-dimethylbutanamido]-3-methoxy-5-methylheptanoyl}pyrrolidin-2-yl]-3-methoxy-2-methylpropanamido}-3-phenylpropanoic acid (58.60 mg, 1 Eq, 11.11 μmol) in DCM (1.5 ml) at 0° C. (ice-water bath) was added pre-cooled TFA (0.5 ml) (TFA/DCM as 25% v/v). The mixture was stirred at 0° for 15 min, then raised to RT and stir at RT for 30 mm. The mixture was concentrated under high vac. for 1 h. To the mixture of the above crude and 2,5-dioxopyrrolidin-1-yl 1-(2,5-dioxo-2,5-dihydro-1H-pyrrol-1-yl)-3,6,9,12-tetraoxapentadecan-15-oate (14.74 mg, 3 Eq, 33.32 μmol) in DMF was added DIPEA (21.53 mg, 29.0 μL, 15 Eq, 166.6 μmol). The reaction mixture was stirred at RT for 30 min. The mixture was loaded directly and separated with C4 column (50 g cartridge, MeCN/Water with 0.05% TFA, 0-80% over 18 CV) to obtain (2S)-2-{(2R,3R)-3-[(2S)-1-{(3R,4S,5S)-4-[(2S)-2-{[(1R,3S,4S)-2-({[4-{[(37S,40S)-23-{2-[2-({1-[(6S,9S)-14-Amino-9-{[4-({[(1R,3S,4S)-3-{[(2S)-1-{[(3R,4S,5S)-3-methoxy-1-{(2S)-2-[(1R,2R)-1-methoxy- 2-methyl-3-oxo-3-{[(1S)-2-phenyl-1-(1,3-thiazol-2-yl)ethyl]amino}propyl]pyrrolidin-1-yl}-5-methyl-1-oxoheptan-4-yl](methyl)amino}-3-methyl-1-oxobutan-2-yl]carbamoyl}-2-azabicyclo[2.2.1]heptane-2-carbonyl]oxy}methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]carbamoyl}-4,7,14-trioxo-6-(propan-2-yl)-3-oxa-5,8,13-triazatetradecan-1-yl]-1H-1,2,3-triazol-4-yl}methoxy)ethoxy]ethyl}-40-(3-carbamamidopropyl)-1-(2,5-dioxo-2,5-dihydro-1H-pyrrol-1-yl)-15,22,35,38,41-pentaoxo-37-(propan-2-yl)-3,6,9,12,19,26,29,32-octaoxa-16,23,36,39-tetraazahentetracontan-41-yl]amino}-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl]methoxy}carbonyl)-2-azabicyclo[2.2.1]heptane-3-carbonyl]amino}-N,3-dimethylbutanamido]-3-methoxy-5-methylheptanoyl}pyrrolidin-2-yl]-3-methoxy-2-methylpropanamido}-3-phenylpropanoic acid (63 mg, 95% yield). HRMS: MH⁺=5501.1196, Rt=2.72 min (5 min acidic method).

### Synthesis of (2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid

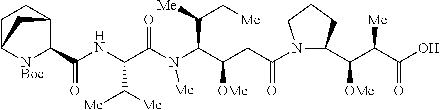 [SMILES: CC[C@H](C)[C@@H]([C@@H](CC(=O)N1CCC[C@H]1[C@H](OC)[C@@H](C)C(=O)O)OC)N(C)C(=O)[C@@H](NC(=O)[C@@H]1[C@H]2CC[C@H](C2)N1C(=O)OC(C)(C)C)C(C)C]

A solution of lithium hydroxide monohydrate (0.26 g, 6.21 mmol) in water (5 mL) was added dropwise to a solution of tert-butyl (1R,3S,4S)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (2.20 g, 3.10 mmol) in tetrahydrofuran (5 mL) and methanol (5 mL). After addition was complete, the mixture was stirred at ambient temperature for 18 hours. The mixture was then quenched with 1 M aqueous HCl (6.5 mL) and volatiles were removed under vacuum. The resulting residue was partitioned between ethyl acetate (50 mL) and brine (100 mL). The layers were separated and the aqueous layer was extracted with ethyl acetate (2×50 mL). The combined organic layers were dried over sodium sulfate, filtered, and concentrated. After drying under vacuum, (2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid was obtained (1.96 g, 2.82 mmol, 91%). LCMS: MH⁺=695.5; Rt=0.73 min (2 min basic method).

### Synthesis of tert-butyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate

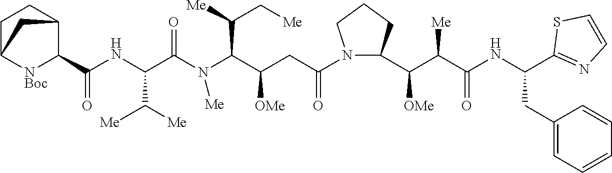 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C(=O)OC(C)(C)C)C(C)C]

To a solution of (2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid (250 mg, 0.360 mmol), (S)-2-phenyl-1-(thiazol-2-yl)ethan-1-amine hydrochloride (95 mg, 0.396 mmol), and (1-[bis(dimethylamino)methylene]-1H-1,2,3-triazolo[4,5-b]pyridinium 3-oxid hexafluorophosphate (150 mg, 0.396 mmol) in DMF (1 mL) was added N,N-diisopropylethylamine (0.25 mL, 1.44 mmol). The resulting mixture was stirred at ambient temperature for 1 hour. The mixture was poured into brine (50 mL) and extracted with ethyl acetate (3×25 mL). The combined organic layers were dried over sodium sulfate, filtered, and concentrated. After purification by SiO₂ ISCO chromatography (0-20% methanol/dichloromethane), tert-butyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate was obtained (317 mg, 99% yield). LCMS: MH⁺=881.5; Rt=1.23 min (2 min basic method).

### Synthesis of (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (P1)

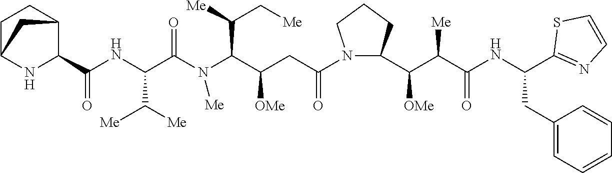 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H]1N[C@@H]2CC[C@H]1C2)C(C)C]

To a solution of tert-butyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (317 mg, 0.360 mmol) in dichloromethane (5 mL) was added trifluoroacetic acid (1 mL). The resulting mixture was stirred at ambient temperature for 1.5 hours, then volatiles were removed under vacuum. The residue was partitioned between ethyl acetate (25 mL) and 1 M aqueous NaOH saturated with NaCl (50 mL). The layers were separated and the aqueous layer was extracted with ethyl acetate (2×25 mL). The combined organic layers were dried over sodium sulfate, filtered and concentrated. After purification by RP-HPLC ISCO gold chromatography (10-100% MeCN/H₂O, 0.1% TFA modifier), (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (P1) was obtained (268 mg, 83% yield). LCMS: MH⁺=781.5; Rt=1.11 min (2 min basic method).

### Synthesis of tert-butyl (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate

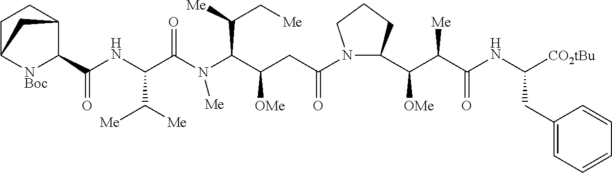 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC(C)(C)C)OC)N(C)C(=O)[C@@H](NC(=O)[C@@H]1[C@H]2CC[C@H](C2)N1C(=O)OC(C)(C)C)C(C)C]

By coupling of (2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid and tert-butyl L-phenylalaninate hydrochloride under standard amide bond conditions, tert-butyl (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate can be prepared.

### Synthesis of ((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine (P2)

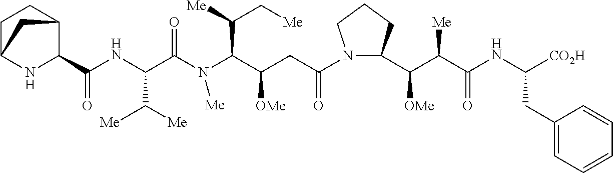 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H]1N[C@@H]2CC[C@H]1C2)C(C)C]

Treatment of tert-butyl (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate with 25% TFA/CH2C2 for one hour, removal of volatiles in vacuo and purification by RP-HPLC can provide ((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoyl)-L-phenylalanine.

### Synthesis of methyl (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoate

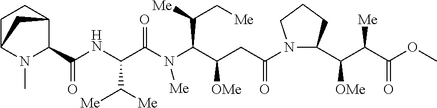 [SMILES: CC[C@H](C)[C@@H]([C@@H](CC(=O)N1CCC[C@H]1[C@H](OC)[C@@H](C)C(=O)OC)OC)N(C)C(=O)[C@@H](NC(=O)[C@@H]1[C@H]2CC[C@H](C2)N1C)C(C)C]

(2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid can be treated with 25% TFA/CH₂Cl₂ for one hour at which time upon removal of volatiles in vacuo, Boc deprotected material can be obtained. Subsequent treatment with paraformaldehde under reductive amination conditions can provide methyl (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoate.

### Synthesis of (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid

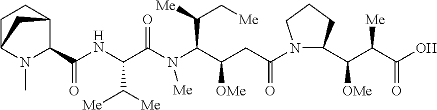 [SMILES: CC[C@H](C)[C@@H]([C@@H](CC(=O)N1CCC[C@H]1[C@H](OC)[C@@H](C)C(=O)O)OC)N(C)C(=O)[C@@H](NC(=O)[C@@H]1[C@H]2CC[C@H](C2)N1C)C(C)C]

Following standard ester hydrolysis conditions and using methyl (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoate, (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid can be prepared.

### Synthesis of (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (P3)

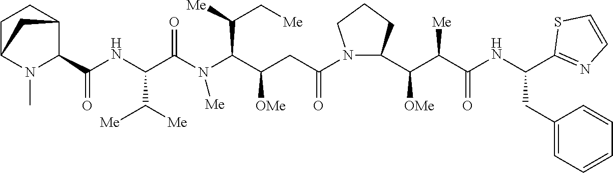 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H]1[C@H]2CC[C@H](C2)N1C)C(C)C]

(1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide can be treated with paraformaldehyde under standard reductive amination conditions to yield (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide.

### Synthesis of tert-butyl ((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoyl)-L-phenylalaninate

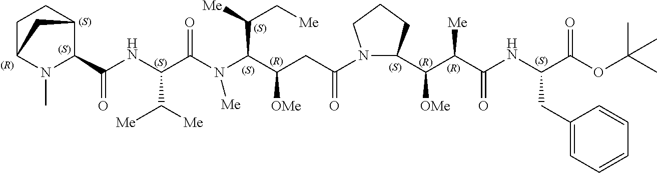

Following standard amide coupling conditions using (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid and tert-butyl L-phenylalaninate hydrochloride, tert-butyl ((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoyl)-L-phenylalaninate can be prepared.

### Synthesis of ((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoyl)-L-phenylalanine (P4)

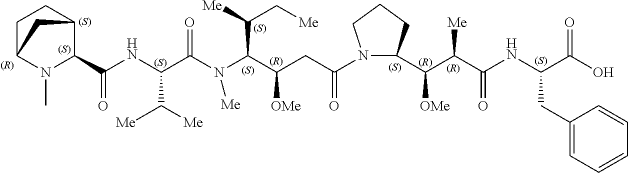

tert-butyl((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoyl)-L-phenylalaninate can be treated with 25% TFA/CH₂Cl₂ for one hour at which time upon removal of volatiles in vacuo and purification by RP-HPLC, ((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoyl)-L-phenylalanine (P4) can be prepared.

### Synthesis of (1R,3S,4S)-2-(4-((S)-2-((S)-2-((tert-butoxycarbonyl)amino)-3-methylbutanamido)-5-ureidopentanamido)-2-((methylamino)methyl)benzyl)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

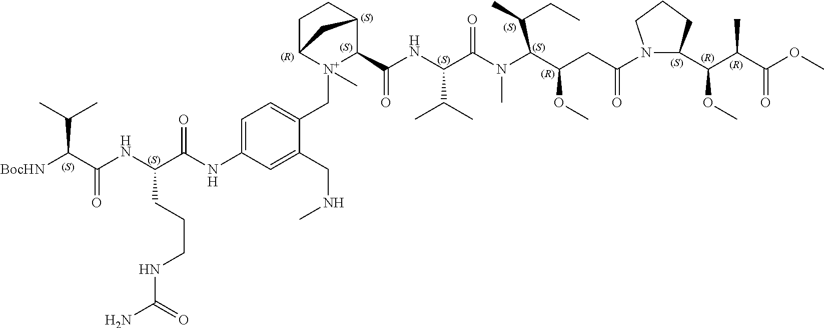

Alkylation of methyl (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoate with (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate in DMSO with added NaI or TBAI followed by in situ FMOC deprotection by treatment with base can, after purification by RP-HPLC, allow for the preparation of (1R,3S,4S)-2-(4-((S)-2-((S)-2-((tert-butoxycarbonyl)amino)-3-methylbutanamido)-5-ureidopentanamido)-2-((methylamino)methyl)benzyl)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate.

### Synthesis of (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

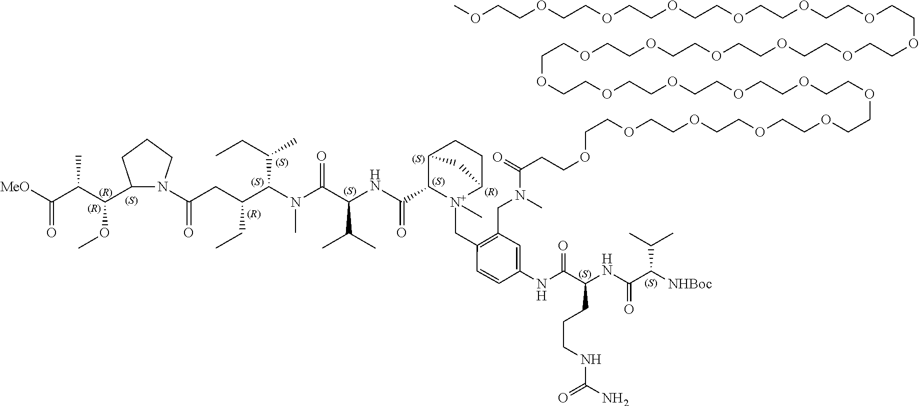

Following standard amide coupling protocols and using 74-oxo-2,5,8,11,14,17,20,23,26,29,32,35,38,41,44,47,50,53,56,59,62,65,68,71-tetracosaoxapentaheptacontan-75-oic acid and (1R,3S,4S)-2-(4-((S)-2-((S)-2-((tert-butoxycarbonyl)amino)-3-methylbutanamido)-5-ureidopentanamido)-2-((methylamino)methyl)benzyl)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate, (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be prepared.

### Synthesis of (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-2-carboxy-1-methoxypropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium

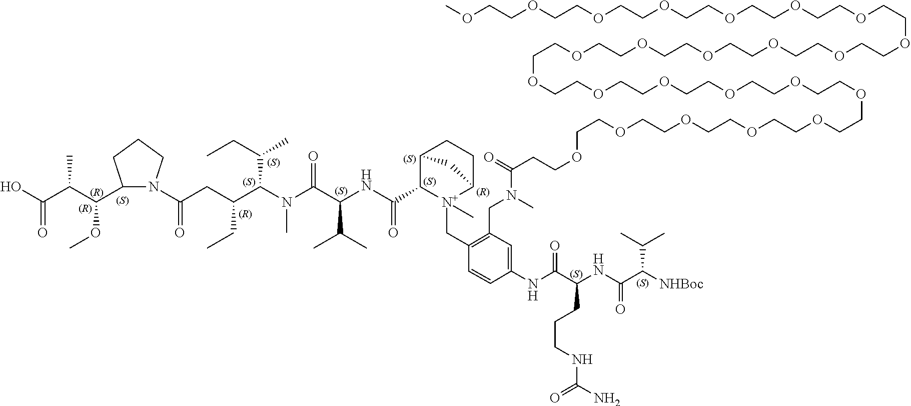

Following standard ester hydrolysis conditions and using (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate, (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-2-carboxy-1-methoxypropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be prepared.

### Synthesis of (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

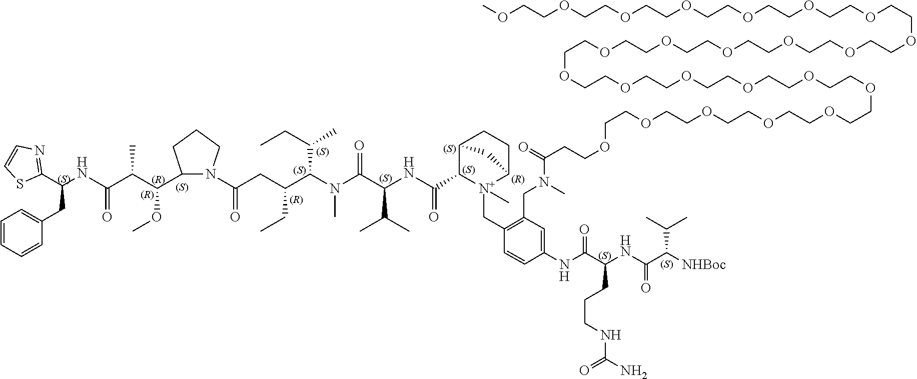

Following standard amide bond coupling conditions and utilizing (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-2-carboxy-1-methoxypropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate and (S)-2-phenyl-1-(thiazol-2-yl)ethan-1-amine hydrochloride, (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be prepared.

### Synthesis of (1R,3S,4S)-2-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

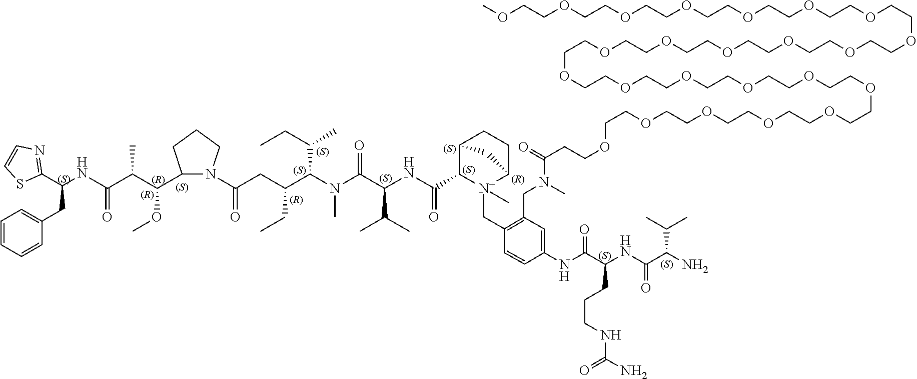

By treatment of (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate with 25% TFA/CH₂Cl₂ for one hour, concentration and purification by RP-HPLC, (1R,3S,4S)-2-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-2-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

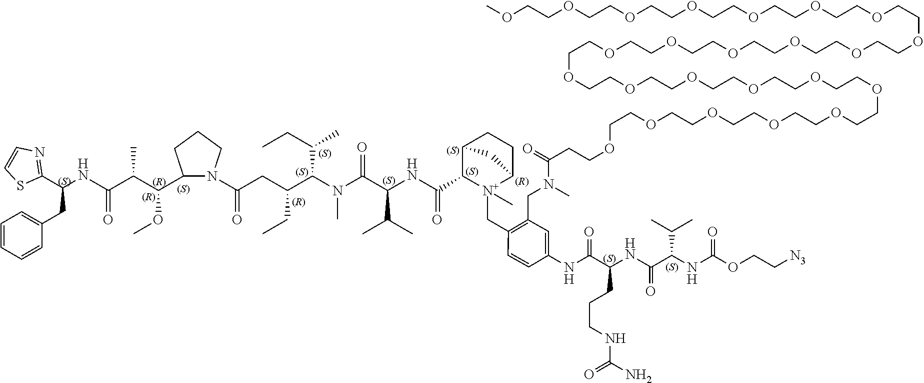

To a solution of (1R,3S,4S)-2-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) and 2-azidoethyl (4-nitrophenyl) carbonate (2 equiv) in DMF (0.2 M) can be added DIEA (3 equiv). After stirring for 2 hours, the solution can be diluted with DMSO and purified by RP-HPLC. Upon lyophilization, (1R,3S,4S)-2-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-methyl-2-azabicyclo[2.2.1]heptan-2-ium trifluoroacetate

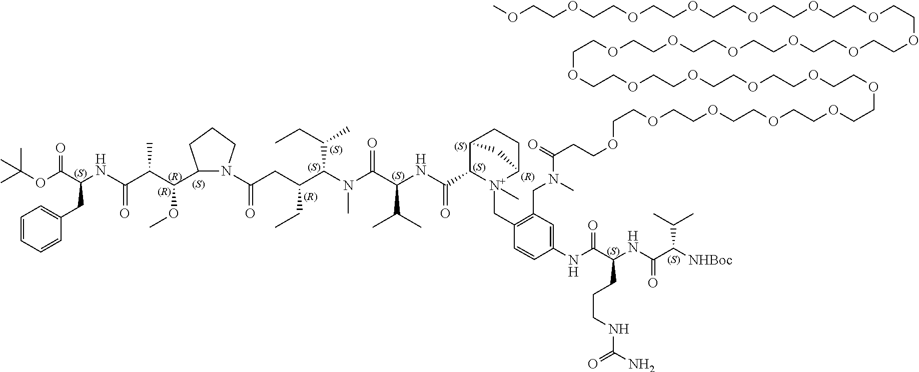

Following standard amide bond coupling conditions and utilizing (1R,3S,4S)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-2-carboxy-1-methoxypropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate and tert-butyl L-phenylalaninate hydrochloride, (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-methyl-2-azabicyclo[2.2.1]heptan-2-ium trifluoroacetate can be prepared.

### Synthesis of (1R,3S,4S)-2-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

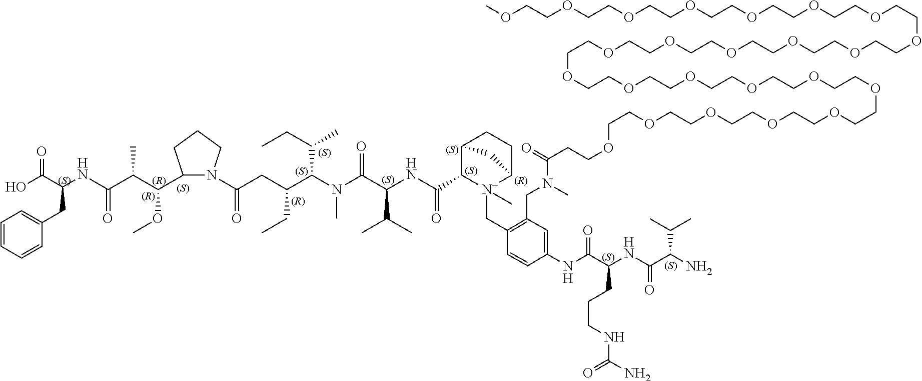

By treatment of (1R,3S,4S)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-methyl-2-azabicyclo[2.2.1]heptan-2-ium trifluoroacetate with 25% TFA/CH₂Cl₂ for one hour, concentration and purification by RP-HPLC, (1R,3S,4S)-2-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be prepared.

### Synthesis of (1R,3S,4S)-2-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

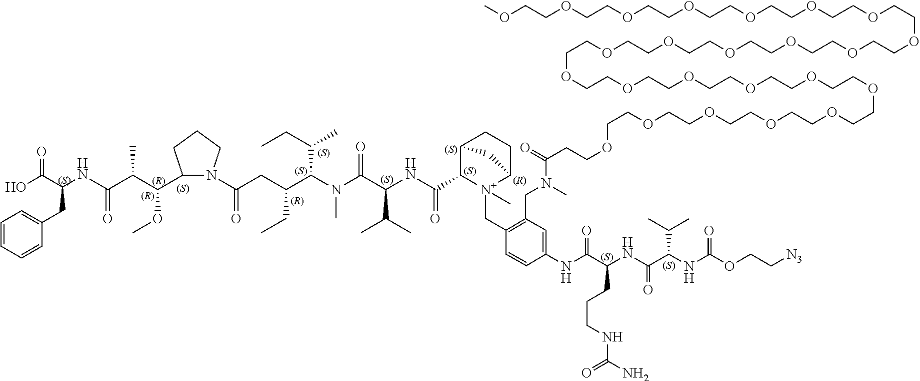

To a solution of (1R,3S,4S)-2-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) and 2-azidoethyl (4-nitrophenyl) carbonate (2 equiv) in DMF (0.2 M) can be added DIEA (3 equiv). After stirring for 2 hours, the solution can be diluted with DMSO and purified by RP-HPLC. Upon lyophilization, (1R,3S,4S)-2-(4-((S)-2-((S)-2-(((2-azidoethoxy)carbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-3-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-2-(4-((S)-2-((S)-2-(((allyloxy)carbonyl)amino)-3-methylbutanamido)-5-ureidopentanamido)-2-((methylamino)methyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

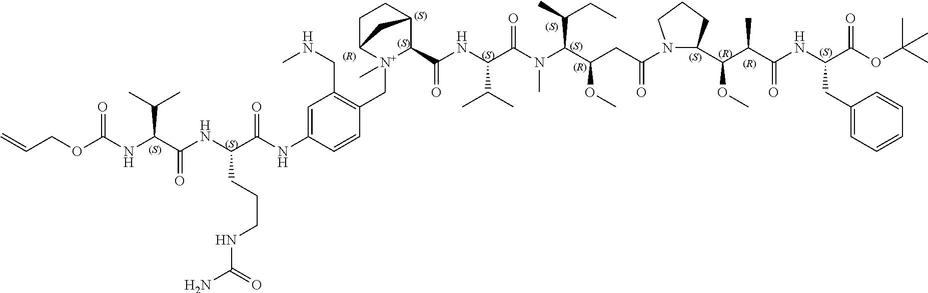

Alkylation of tert-butyl ((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoyl)-L-phenylalaninate with (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate in DMSO with added NaI or TBAI followed by in situ FMOC deprotection by treatment with base can, after purification by RP-HPLC, provide (1R,3S,4S)-2-(4-((S)-2-((S)-2-(((allyloxy)carbonyl)amino)-3-methylbutanamido)-5-ureidopentanamido)-2-((methylamino)methyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate.

### Synthesis of (1R,3S,4S)-2-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

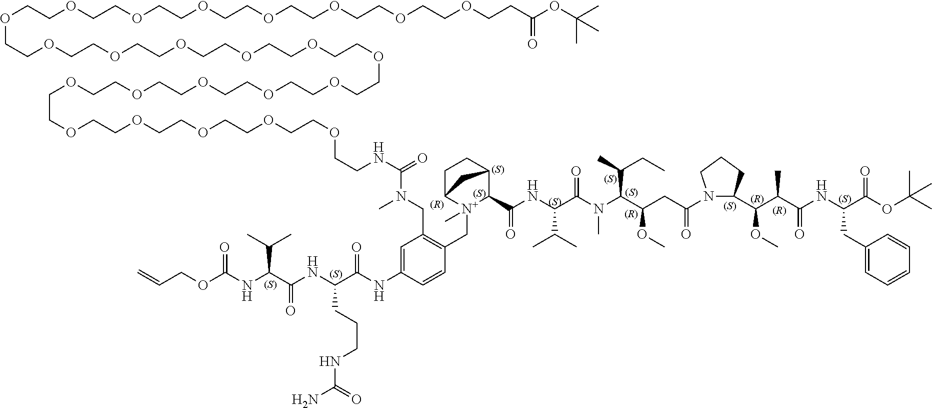

To a solution of bis(4-nitrophenyl) carbonate (1.1 equiv), tert-butyl 1-amino-3,6,9,12,15,18,21,24,27,30,33,36,39,42,45,48,51,54,57,60,63,66,69,72-tetracosaoxapentaheptacontan-75-oate (1.1 equiv) in DMF (0.1 M) can be added DIEA (4 equiv). After stirring for 1 hour, (1R,3S,4S)-2-(4-((S)-2-((S)-2-(((allyloxy)carbonyl)amino)-3-methylbutanamido)-5-ureidopentanamido)-2-((methylamino)methyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) can be added to the solution. After stirring for an additional 2 hours, the solution can be diluted with DMSO and purified by ISCO RP-HPLC. Upon lyophilization, (1R,3S,4S)-2-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-2-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

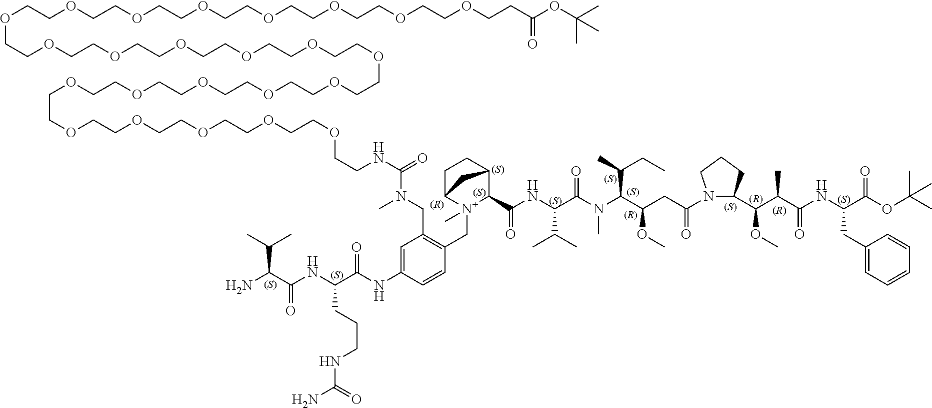

To a solution of (1R,3S,4S)-2-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) in THF (0.1M) can be added N,N,1,1,1-pentamethylsilanamine (5 equiv.). After degassing and purging to N2, tetrakis(triphenylphosphine)palladium (0.15 equiv) can be added, followed by degassing/purging to N2 as above (3×). After stirring for one hour, removal of volatiles in vacuo and purification by RP-HPLC, (1R,3S,4S)-2-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-2-(4-((S)-2-((S)-2-(((2-azidoethoxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

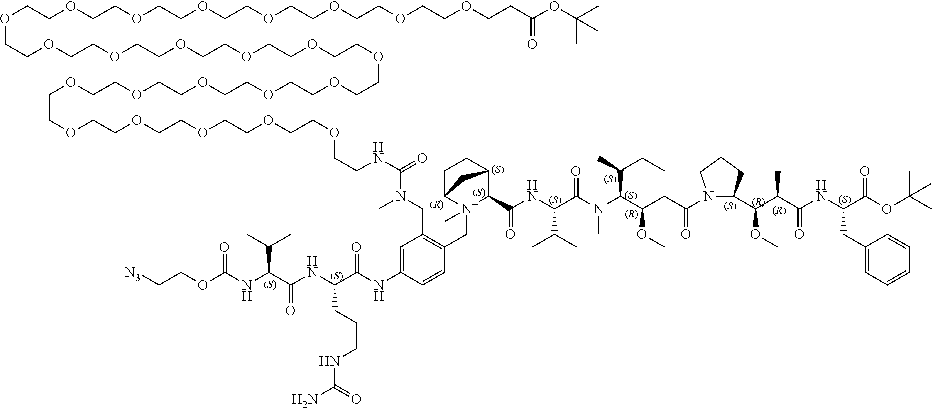

To a solution of (1R,3S,4S)-2-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) and 2-azidoethyl (4-nitrophenyl) carbonate (2 equiv) in DMF (0.2 M) can be added DIEA (3 equiv). After stirring for 2 hours, the solution can be diluted with DMSO and purified by RP-HPLC. Upon lyophilization, (1R,3S,4S)-2-(4-((S)-2-((S)-2-(((2-azidoethoxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-2-(4-((S)-2-((S)-2-(((allyloxy)carbonyl)amino)-3-methylbutanamido)-5-ureidopentanamido)-2-((methylamino)methyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

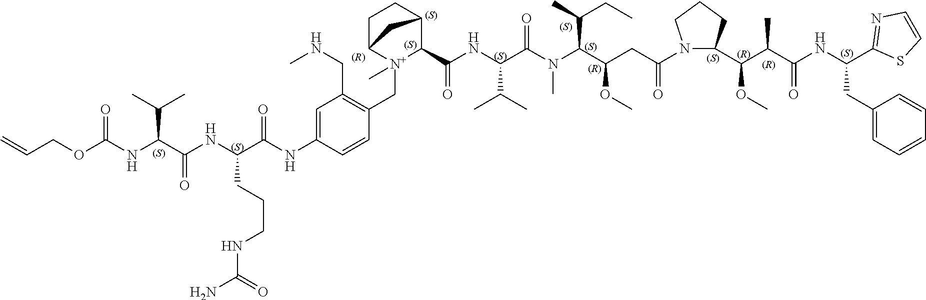

Alkylation of (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide with (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate in DMSO with added NaI or TBAI followed by in situ FMOC deprotection by treatment with base can, after purification by RP-HPLC, provide (1R,3S,4S)-2-(4-((S)-2-((S)-2-(((allyloxy)carbonyl)amino)-3-methylbutanamido)-5-ureidopentanamido)-2-((methylamino)methyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate.

### Synthesis of (1R,3S,4S)-2-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

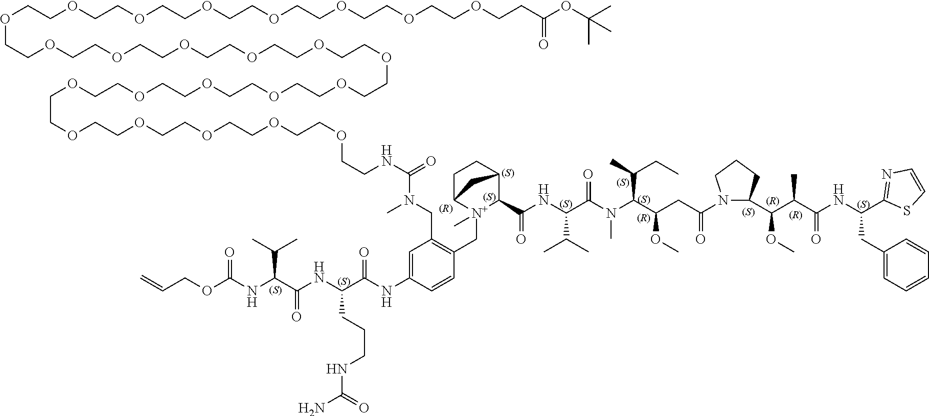

To a solution of bis(4-nitrophenyl) carbonate (1.1 equiv), tert-butyl 1-amino-3,6,9,12,15,18,21,24,27,30,33,36,39,42,45,48,51,54,57,60,63,66,69,72-tetracosaoxapentaheptacontan-75-oate (1.1 equiv) in DMF (0.1 M) can be added DIEA (4 equiv). After stirring for 1 hour, (1R,3S,4S)-2-(4-((S)-2-((S)-2-(((allyloxy)carbonyl)amino)-3-methylbutanamido)-5-ureidopentanamido)-2-((methylamino)methyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) can be added to the solution. After stirring for an additional 2 hours, the solution can be diluted with DMSO and purified by ISCO RP-HPLC. Upon lyophilization, (1R,3S,4S)-2-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-2-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

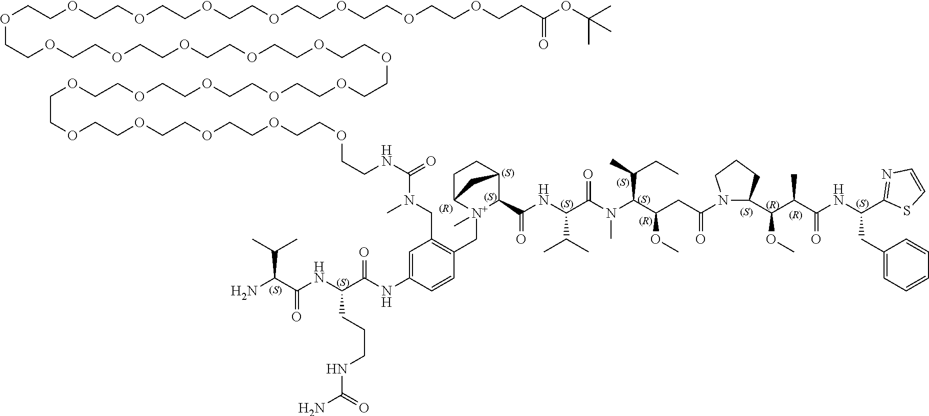

To a solution of (1R,3S,4S)-2-(4-((S)-2-((S)-2-(((allyloxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) in THF (0.1M) can be added N,N,1,1,1-pentamethylsilanamine (5 equiv.). After degassing and purging to N2, tetrakis(triphenylphosphine)palladium (0.15 equiv) can be added, followed by degassing/purging to N2 as above (3×). After stirring for one hour, removal of volatiles in vacuo and purification by RP-HPLC, (1R,3S,4S)-2-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of (1R,3S,4S)-2-(4-((S)-2-((S)-2-(((2-azidoethoxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate

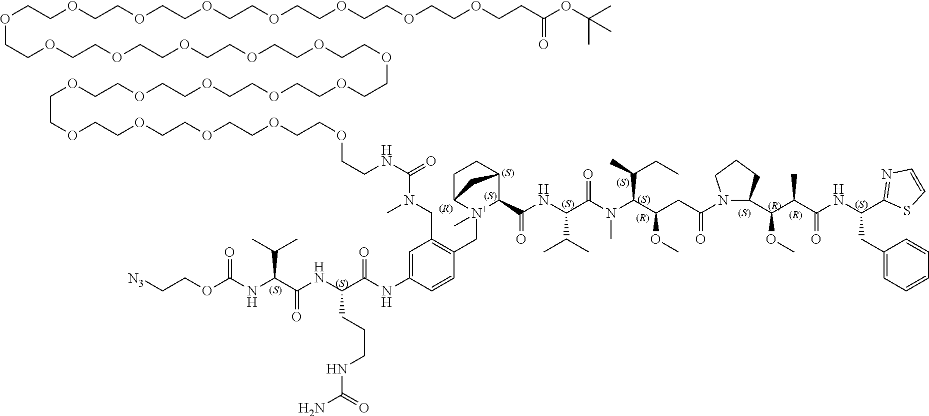

To a solution of (1R,3S,4S)-2-(4-((S)-2-((S)-2-amino-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate (1 equiv) and 2-azidoethyl (4-nitrophenyl) carbonate (2 equiv) in DMF (0.2 M) can be added DIEA (3 equiv). After stirring for 2 hours, the solution can be diluted with DMSO and purified by RP-HPLC. Upon lyophilization, (1R,3S,4S)-2-(4-((S)-2-((S)-2-(((2-azidoethoxy)carbonyl)amino)-3-methylbutanamido)-5-ureidopentanamido)-2-(2,81,81-trimethyl-3,79-dioxo-7,10,13,16,19,22,25,28,31,34,37,40,43,46,49,52,55,58,61,64,67,70,73,76,80-pentacosaoxa-2,4-diazadooctacontyl)benzyl)-3-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-methyl-2-azabicyclo[2.2.1]heptan-2-ium trifluoroacetate can be obtained.

### Synthesis of methyl (2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoate

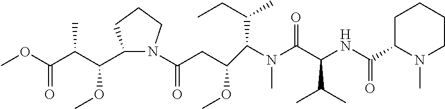 [SMILES: CC[C@H](C)[C@@H]([C@@H](CC(=O)N1CCC[C@H]1[C@H](OC)[C@@H](C)C(=O)OC)OC)N(C)C(=O)[C@@H](NC(=O)[C@@H]1CCCCN1C)C(C)C]

To (S)-1-methylpiperidine-2-carboxylic acid (585 mg, 4.09 mmol) in 1:2 MeOH/DMF (9 mL) was added and HATU (1.553 g, 4.09 mmol) and DIEA (2.28 mL, 13.07 mmol). After stirring 15 minutes, methyl (2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoate (CAS #1816949-65-1) (1587 mg, 3.27 mmol) was added. After stirring for 10 minutes the solution was diluted with DMSO and purified by ISCO RP-HPLC. Upon lyophilization, methyl (2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoate (1995 mg, 84%) was obtained as the TFA salt. HRMS: MH⁺=611.4600, Rt=1.71 min (5 min acidic method).

### Synthesis of (2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoic acid

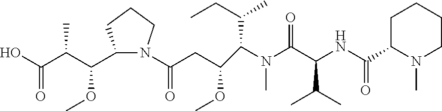 [SMILES: CC[C@H](C)[C@@H]([C@@H](CC(=O)N1CCC[C@H]1[C@H](OC)[C@@H](C)C(=O)O)OC)N(C)C(=O)[C@@H](NC(=O)[C@@H]1CCCCN1C)C(C)C]

To (methyl (2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoate TFA salt (1.0 g, 1.637 mmol) in 12 mL 1:1:1 MeOH/THF/H2O was added LiOH (784 mg, 32.74 mmol). After stirring for 1 hour the solution was neutralized by addition of 6N HCl (5.46 mL, 32.74 mmol), the volatiles were removed in vacuo, the residue was dissolved in DMSO and was purified by ISCO RP-HPLC. Upon lyophilization, (2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoic acid (581 mg, 49.9% yield) was obtained as the TFA salt. HRMS: M+Na⁺=619.4231, Rt=1.48 min (5 min acidic method).

### Synthesis of tert-butyl ((2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoyl)-L-phenylalaninate

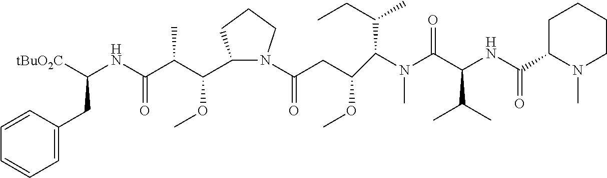 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC(C)(C)C)OC)N(C)C(=O)[C@@H](NC(=O)[C@@H]1CCCCN1C)C(C)C]

To (2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoic acid TFA salt (692.4 mg, 1.16 mmol) in DMF (3 mL) was added and HATU (485.3 mg, 1.276 mmol) and DIEA (1.21 mL, 6.96 mmol). After stirring 15 minutes, tert-butyl L-phenylalaninate (308 mg, 1.392 mmol) and additional DIEA (1.21 mL, 6.96 mmol) were added. After stirring for 1 hour the solution was diluted with DMSO and purified by ISCO RP-HPLC. Upon lyophilization, tert-butyl ((2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoyl)-L-phenylalaninate (741 mg, 70%) was obtained as the TFA salt. HRMS: MH⁺=800.5700, Rt=2.18 min (5 min acidic method).

### Synthesis of (2S)-2-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-(4-((S)-2-((S)-2-((tert-butoxycarbonyl)amino)-3-methylbutanamido)-5-ureidopentanamido)-2-((methylamino)methyl)benzyl)-1-methylpiperidin-1-ium trifluoroacetate

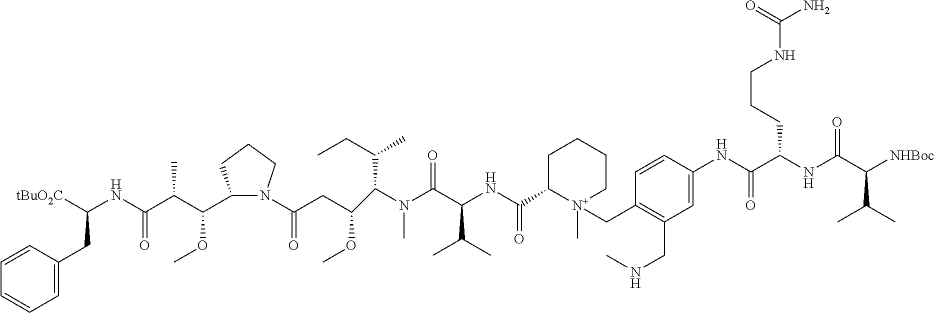 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC(C)(C)C)OC)N(C)C(=O)[C@@H](NC(=O)[C@@H]1CCCC[N+]1(C)Cc1ccc(NC(=O)[C@H](CCCNC(N)=O)NC(=O)[C@@H](NC(=O)OC(C)(C)C)C(C)C)cc1CNC)C(C)C]

To a solution of tert-butyl ((2R,3R)-3-((S)-1-((3R,4S,5S)-4-((S)—N,3-dimethyl-2-((S)-1-methylpiperidine-2-carboxamido)butanamido)-3-methoxy-5-methylheptanoyl)pyrrolidin-2-yl)-3-methoxy-2-methylpropanoyl)-L-phenylalaninate trifluoroacetate (741.7, 841.6 μmol), NaI (243.2 mg, 1683 μmol) and (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (681.3 mg, 892.5 μmol) in DMSO (10 mL) was added DIEA (848 μL, 4.868 mmol). After stirring for 14 hours diethyl amine (1.68 mL, 16.23 mmol) was added. After stirring for 30 minutes the solution was purified by RP-HPLC. After lyophilization, (2S)-2-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-(4-((S)-2-((S)-2-((tert-butoxycarbonyl)amino)-3-methylbutanamido)-5-ureidopentanamido)-2-((methylamino)methyl)benzyl)-1-methylpiperidin-1-ium trifluoroacetate (536 mg, 46% yield) was obtained. HRMS: M+=1304.8800, Rt=2.04 min (5 min acidic method).

### Synthesis of (2S)-2-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperidin-1-ium trifluoroacetate

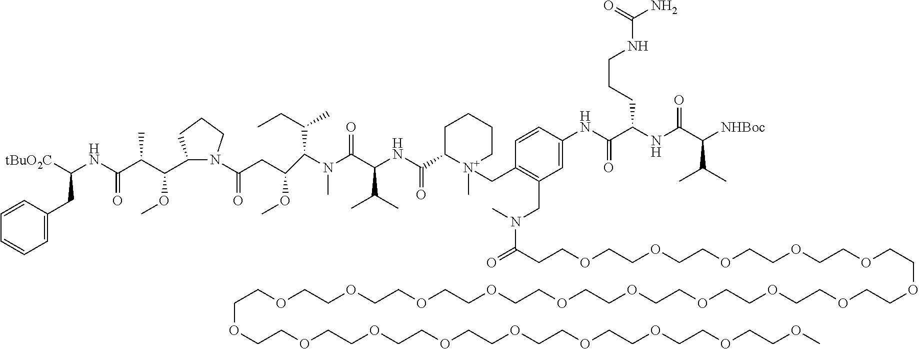 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC(C)(C)C)OC)N(C)C(=O)[C@@H](NC(=O)[C@@H]1CCCC[N+]1(C)Cc1ccc(NC(=O)[C@H](CCCNC(N)=O)NC(=O)[C@@H](NC(=O)OC(C)(C)C)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC)C(C)C]

To 2,5,8,11,14,17,20,23,26,29,32,35,38,41,44,47,50,53,56,59,62,65,68,71-tetracosaoxatetraheptacontan-74-oic acid (351.9 mg, 314.9 μmol) in DMF (2 ml) was added HATU (119.7 mg, 314.9 μmol) followed by DIEA (274 μL, 1575 mmol). The mixture was stirred for 30 min. To the above mixture was added a solution of (2S)-2-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-(4-((S)-2-((S)-2-((tert-butoxycarbonyl)amino)-3-methylbutanamido)-5-ureidopentanamido)-2-((methylamino)methyl)benzyl)-1-methylpiperidin-1-ium trifluoroacetate (372.6 mg, 262.4 μmol) in DMF (2 ml) was added. The mixture was stirred at Rt for 2 h. The solution was diluted with DMSO and purified by ISCO gold RP-HPLC. Upon lyophilization, (2S)-2-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperidin-1-ium trifluoroacetate (570 mg, 90% yield) was obtained. HRMS: M⁺=2403.4900, Rt=2.43 min (5 min acidic method).

### Synthesis of (2S)-1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate

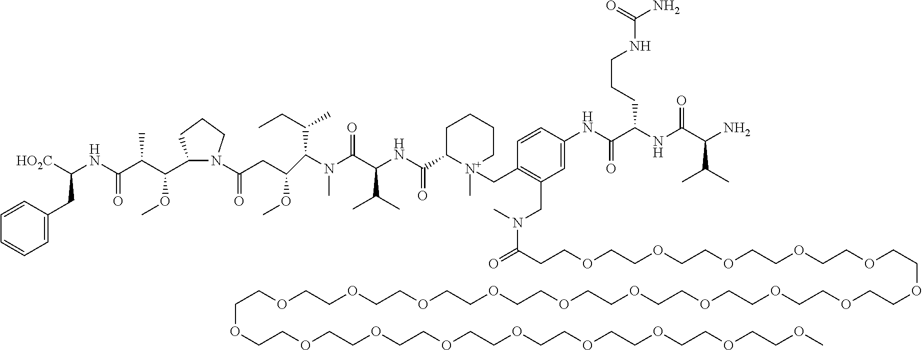 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1CCCC[N+]1(C)Cc1ccc(NC(=O)[C@H](CCCNC(N)=O)NC(=O)[C@@H](N)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC)C(C)C]

(2S)-2-(((S)-1-(((3R,4S,5S)-1-((S)-2-((1R,2R)-3-(((S)-1-(tert-butoxy)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-methylpiperidin-1-ium trifluoroacetate (570 mg, 237 μmol) was treated with 25% TFA/CH2Cl2 with 1% Et3SiH for 2 hours at which the volatiles were removed in vacuo. The residue was dissolved in DMSO and purified by ISCO gold RP-HPLC. Upon lyophilization, (2S)-1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate TFA salt (400 mg, 71% yield) was obtained. HRMS: M+=2248.3701, Rt=1.80 min (5 min acidic method).

### Synthesis of (2S,2′S)-1,1′-(((((2S,5S,39S,42S)-22-(3-(2-((((9H-fluoren-9-yl)methoxy)carbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate

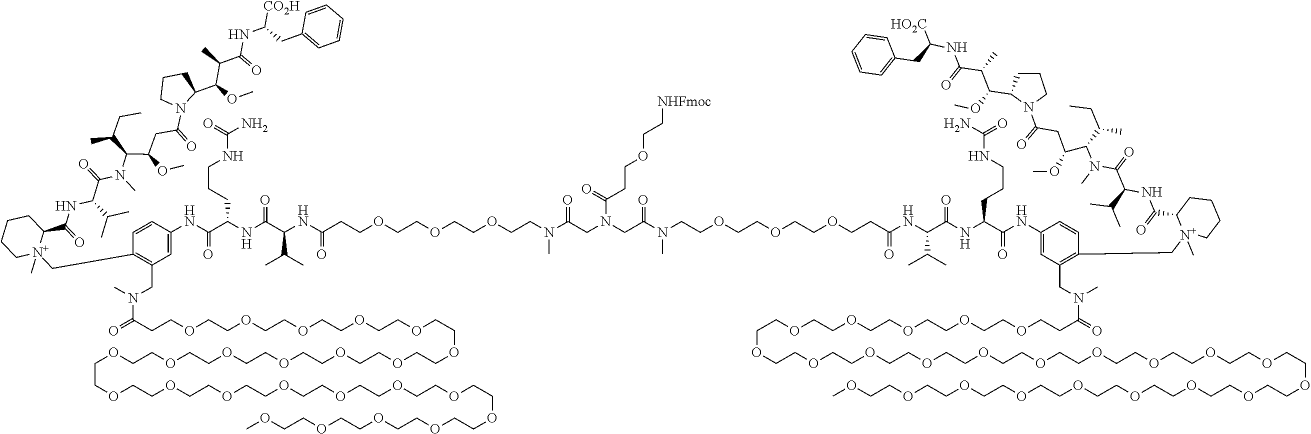

To a solution of (2S)-1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate (76.6 mg, 32.4 μmol) and bis(2,5-dioxopyrrolidin-1-yl) 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate (15.5 mg, 14.1 μmol) in DMF (0.8 mL) was added DIEA (24.6 uL, 141 μmol). After 16 hours the solution was diluted with DMSO and purified by ISCO RP-HPLC. Upon lyophilization, (2S,2′S)-1,1′-(((((2S,5S,39S,42S)-22-(3-(2-((((9H-fluoren-9-yl)methoxy)carbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate (48.6 mg, 64% yield) was obtained. HRMS: (M⁺2-H⁺)⁺=5362.1899, Rt=2.35 min (5 min acidic method).

### Synthesis of (2S,2′S)-1,1′-(((((2S,5S,39S,42S)-22-(3-(2-aminoethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate

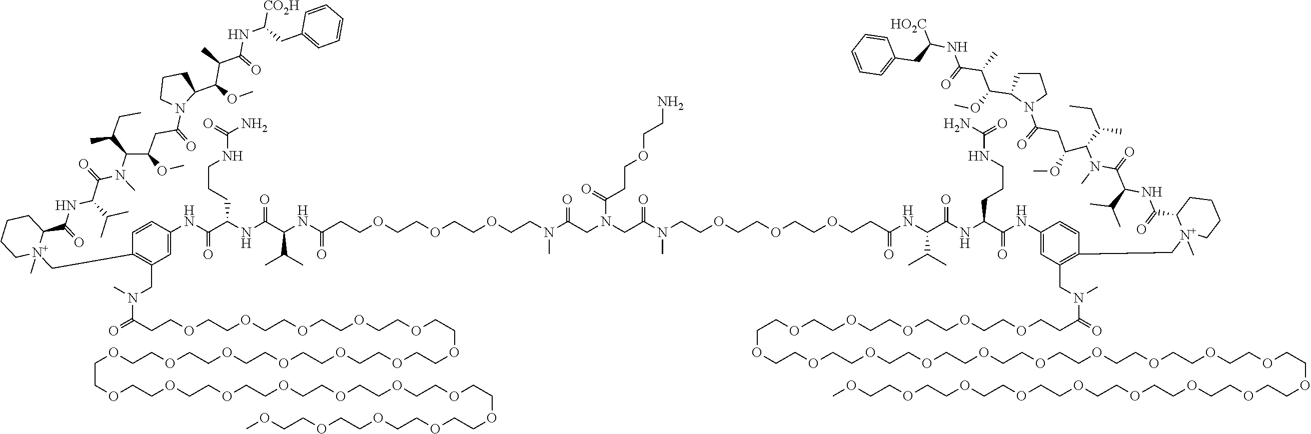

A solution of (2S,2′S)-1,1′-(((((2S,5S,39S,42S)-22-(3-(2-((((9H-fluoren-9-yl)methoxy)carbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate (46.2 mg, 8.6 μmol) in DMF was treated with Et2NH (17.8 uL, 172.2 μmol) for 1 hour at which time the solution was diluted with DMSO and purified by ISCO RP-HPLC. Upon lyophilization, (2S,2′S)-1,1′-(((((2S,5S,39S,42S)-22-(3-(2-aminoethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate (34.5 mg, 76% yield) was obtained. HRMS: (M⁺²—H⁺)⁺=5140.1201, Rt=2.06 min (5 min acidic method).

### Example #5: Synthesis of (2S,2′S)-1,1′-(((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate, P5-L5-P5

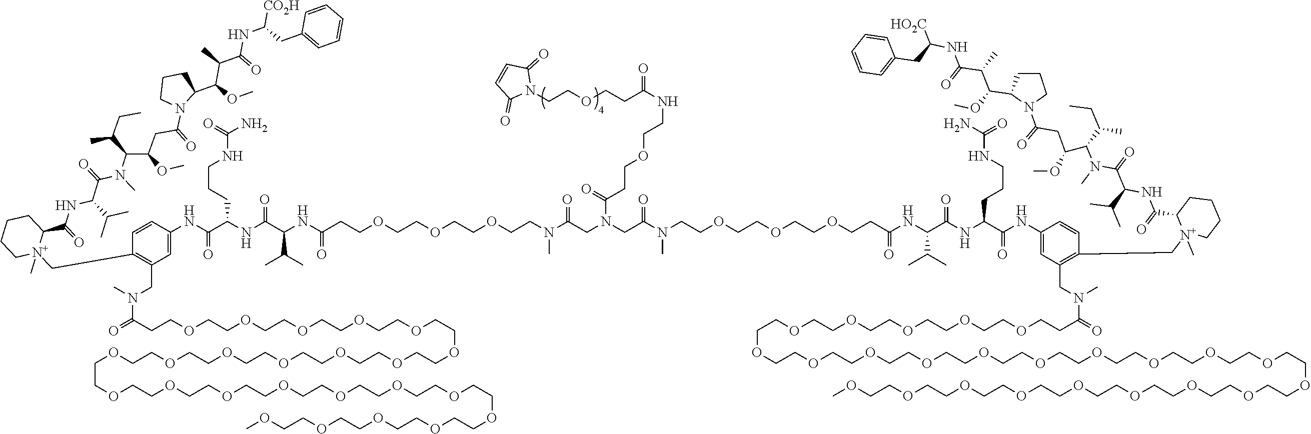

To a solution of (2S,2′S)-1,1′-(((((2S,5S,39S,42S)-22-(3-(2-aminoethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate (41.3 mg, 8.0 μmol) and 2,5-dioxopyrrolidin-1-yl 1-(2,5-dioxo-2,5-dihydro-1H-pyrrol-1-yl)-3,6,9,12-tetraoxapentadecan-15-oate (7.1 mg, 16.1 μmol) in DMF was added DIEA (21 μL, 120.4 μmol). After standing for 3 hours the solution was diluted with DMSO and purified by RP-HPLC. Upon lyophilization, (2S,2′S)-1,1′-(((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(2-(75-methyl-74-oxo-2,5,8,11,14,17,20,23,26,29,32,35,38,41,44,47,50,53,56,59,62,65,68,71-tetracosaoxa-75-azahexaheptacontan-76-yl)-4,1-phenylene))bis(methylene))bis(2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium) trifluoroacetate (31 mg, 76% yield) was obtained. HRMS: (M⁺²—H⁺)⁺=5467.2200, Rt=2.25 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate

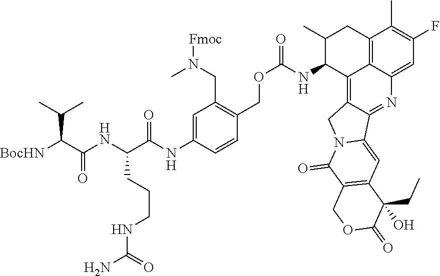

To a solution of (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione mesylate salt (1.5 g, 2,822 mmol) and (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (2.44 g, 2.68 mmol) in NMP (10 mL) was added DIEA (1133 μL, 7.07 mmol). After standing for 8 hours the solution was diluted with DMSO and purified by RP-HPLC with 0.05% formic acid modifier. After lyophilization, (9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (2.197 g, 64% yield) was obtained. HRMS: MH⁺=1206.5500, Rt=2.87 min (5 min acidic method).

### Synthesis of (9H-fluoren-9-yl)methyl (5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate

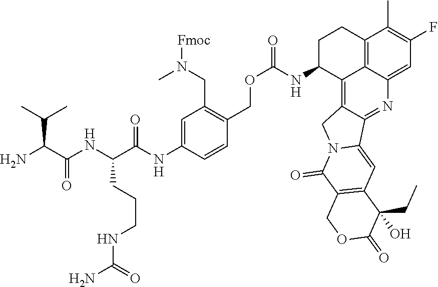

(9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (2.197 g, 1.821 mmol) was treated with 20 mL of 25% TFA/CH₂Cl₂ with 1% Et₃SiH for one hour at which time the volatiles were removed in vacuo. The residue was triturated with Et₂O and pumped on to yield 2.07 grams (9H-fluoren-9-yl)methyl (5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate. The material was used directly as is. HRMS: MH⁺=1106.5000, Rt=2.09 min (5 min acidic method).

### Synthesis of allyl (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oate

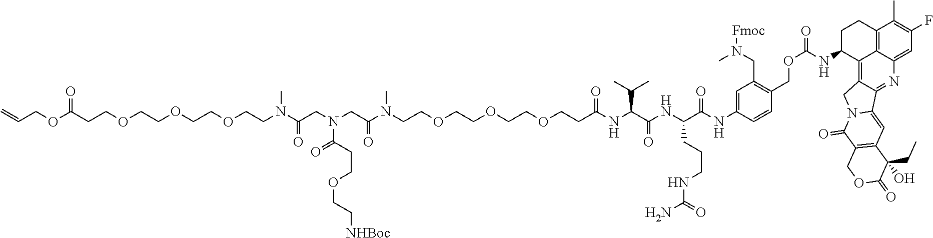

To a solution of (9H-fluoren-9-yl)methyl (5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate TFA salt (2.07 g, 1696 μmol) and 16-(3-(2-((tert-butoxycarbonyl)amino)ethoxy)propanoyl)-13,19-dimethyl-14,18,31-trioxo-4,7,10,22,25,28,32-heptaoxa-13,16,19-triazapentatriacont-34-enoic acid (1.565 g, 1902 μmol) in NMP (12 mL) was added HOAT (346.3 mg, 2545 μmol), DIEA (1.33 mL, 7634 μmol) and PyBOP (1.324 g, 2545 μmol). After stirring for 2 hours the solution was diluted with DMSO and purified by RP-HPLC. Upon lyophilization, allyl (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oate (1.80 g, 55% yield) was obtained. HRMS: MH+=1910.9200, Rt=1.85 min (5 min acidic method).

### Synthesis of (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oic acid

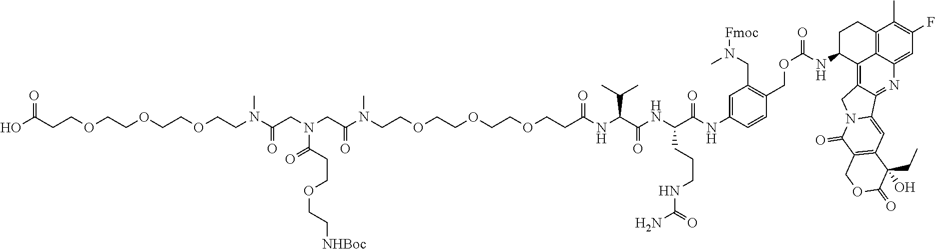

To a solution of allyl (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oate (1.80 g, 942 μmol) and phenyl silane (152.9 mg, 1413 μmol) in 1:1 CH₂Cl₂/MeOH (14 mL) under N2 atmosphere was added tetrakis(triphenylphosphine)palladium (16.3 mg, 14.13 μmol). After stirring for 2 hours the volatiles were removed in vacuo, the residue was dissolved in DMSO and was purified by RP-HPLC. Upon lyophilization, (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oic acid (1.65 g, 93.6% yield) was obtained. HRMS: MH+=1870.8900, Rt=2.63 min (5 min acidic method).

### Synthesis of (6S,9S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-((methylamino)methyl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid

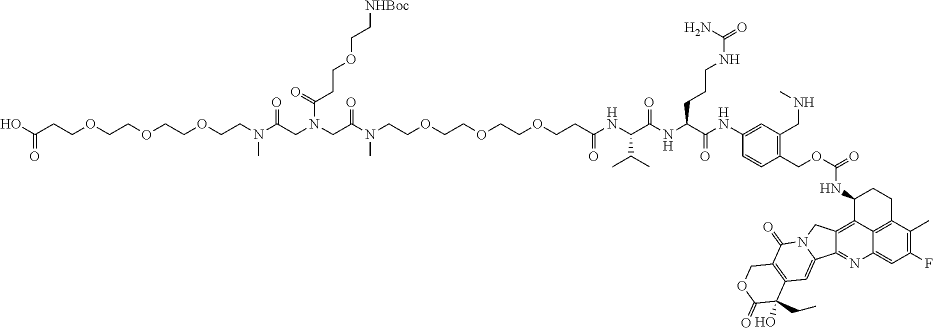 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCOCCOCCOCCN(C)C(=O)CN(CC(=O)N(C)CCOCCOCCOCCC(=O)O)C(=O)CCOCCNC(=O)OC(C)(C)C)C(C)C)cc1CNC)CC3]

To a solution of (6S,9S)-6-((3-(((((9H-fluoren-9-yl)methoxy)carbonyl)(methyl)amino)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-1-amino-26-(3-(2-((tert-butoxycarbonyl)amino)ethoxy)propanoyl)-9-isopropyl-23,29-dimethyl-1,8,11,24,28-pentaoxo-14,17,20,32,35,38-hexaoxa-2,7,10,23,26,29-hexaazahentetracontan-41-oic acid (978 mg, 523 μmol) in DMF (6 mL) was added piperidine (258 μL, 2615 μmol). After standing for 2 hours the solution was diluted with DMSO and purified by RP-HPLC. Upon lyophilization, (6S,9S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-((methylamino)methyl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid (501 mg, 54% yield) was obtained as the TFA salt. HRMS: MH+=1648.8199, Rt=1.92 min (5 min acidic method).

### Synthesis of (6S,9S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid

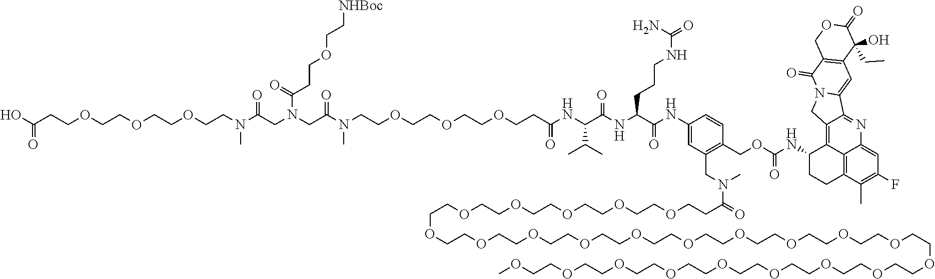 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCOCCOCCOCCN(C)C(=O)CN(CC(=O)N(C)CCOCCOCCOCCC(=O)O)C(=O)CCOCCNC(=O)OC(C)(C)C)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC)CC3]

To a solution of (6S,9S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-((methylamino)methyl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid TFA salt (501 mg, 284.2 μmol) and 2,5-dioxopyrrolidin-1-yl 2,5,8,11,14,17,20,23,26,29,32,35,38,41,44,47,50,53,56,59,62,65,68,71-tetracosaoxatetraheptacontan-74-oate (483.2 mg, 398 μmol) in NMP (4 mL) was added DIEA (99 μL, 568.4 μmol). After standing for 22 hours the solution was diluted with DMSO and purified by RP-HPLC. Upon lyophilization, (6S,9S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid (582 mg, 74% yield) was obtained. HRMS: MH+=2747.4600, Rt=2.32 min (5 min acidic method).

### Synthesis of (2S)-1-(4-((6S,9S,43S,46S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate

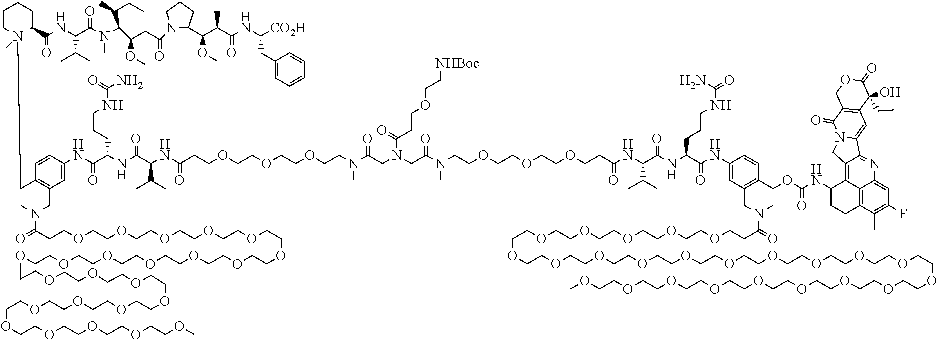

To a solution of (6S,9S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9-isopropyl-23,29-dimethyl-1,8,11,24,28-pentaoxo-14,17,20,32,35,38-hexaoxa-2,7,10,23,26,29-hexaazahentetracontan-41-oic acid (207 mg, 75.2 μmol) in NMP (2 mL) was added DIEA (46 μL, 263.6 μmol) and then TSTU (22.7 mg, 75.2 μmol). After stirring for 45 minutes this NHS ester activated acid solution was added to a NMP solution of (2S)-1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate (223.9 mg, 90.4 μmol) with added DIEA (46 μL, 263.6 μmol). After standing for 7 hours the solution was diluted with DMSO and purified by RP-HPLC. Upon lyophilization, (2S)-1-(4-((6S,9S,43S,46S)-1-amino-26-(3-(2-((tert-butoxycarbonyl)amino)ethoxy)propanoyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate (250 mg, 55% yield) was obtained. HRMS: M+=4975.8301, Rt=2.37 min (5 min acidic method).

### Example #6: Synthesis of (2S)-1-(4-((6S,9S,43S,46S)-1-amino-26-(1-(2,5-dioxo-2,5-dihydro-1H-pyrrol-1-yl)-15-oxo-3,6,9,12,19-pentaoxa-16-azadocosan-22-oyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate, P5-L5-P7

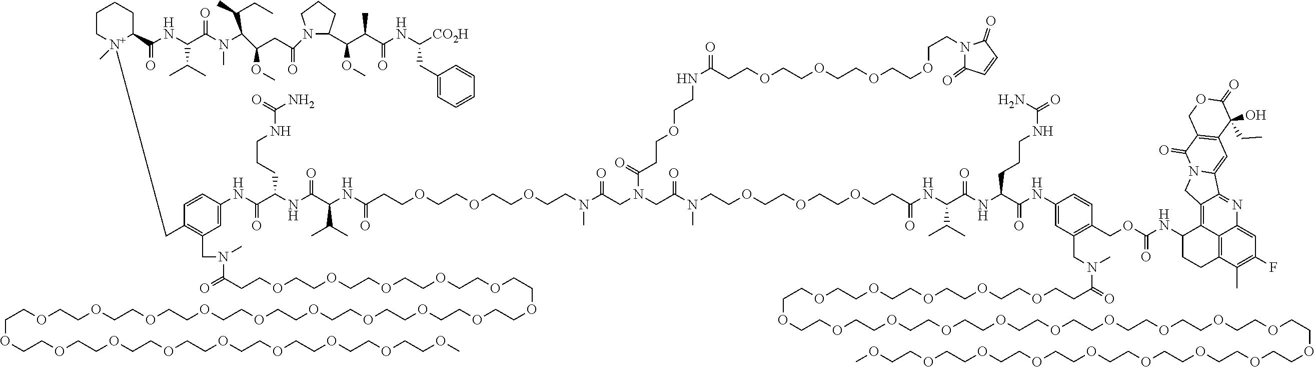

(2S)-1-(4-((6S,9S,43S,46S)-1-amino-26-(3-(2-((tert-butoxycarbonyl)amino)ethoxy)propanoyl)-6-((4-(((((1 S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate (78 mg, 15.3 μmol) was treated with 6 mL of 25% TFA/CH2CL2 with 1% Et3SiH for one hour at which time the volatiles were removed in vacuo and the residue was triturated with Et2O. The resulting residue was dissolved in NMP (2 mL) and was treated with 2,5-dioxopyrrolidin-1-yl 1-(2,5-dioxo-2,5-dihydro-1H-pyrrol-1-yl)-3,6,9,12-tetraoxapentadecan-15-oate (10.2 mg, 23.0 μmol) and DIEA (18.7 μL, 107.2 μmol). After standing for 2 hours the solution was diluted with DMSO and purified by RP-HPLC. Upon lyophilization, (2S)-1-(4-((6S,9S,43S,46S)-1-amino-26-(1-(2,5-dioxo-2,5-dihydro-1H-pyrrol-1-yl)-15-oxo-3,6,9,12,19-pentaoxa-16-azadocosan-22-oyl)-6-((4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3-(75-methyl-74-oxo-2,5,8,11,14,17,20,23,26,29,32,35,38,41,44,47,50,53,56,59,62,65,68,71-tetracosaoxa-75-azahexaheptacontan-76-yl)phenyl)carbamoyl)-9,43-diisopropyl-23,29-dimethyl-1,8,11,24,28,41,44-heptaoxo-46-(3-ureidopropyl)-14,17,20,32,35,38-hexaoxa-2,7,10,23,26,29,42,45-octaazaheptatetracontan-47-amido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-2-(((S)-1-(((3R,4S,5S)-1-((S)-2-((1R,2R)-3-(((S)-1-carboxy-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-1-methylpiperidin-1-ium trifluoroacetate (25 mg, 28% yield) was obtained. HRMS: M⁺=5202.9102, Rt=2.37 min (5 min acidic method).

### Synthesis of 4-((S)-2-((S)-2-((tert-butoxycarbonyl)amino)-3-methylbutanamido)-5-ureidopentanamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate

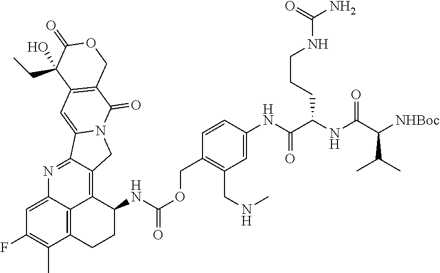 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)OC(C)(C)C)C(C)C)cc1CNC)CC3]

To a solution of (9H-fluoren-9-yl)methyl (5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (231 mg, 191.5 μmol) in DMF (4 mL) was added piperidine (189 μL, 1915 μmol). After standing for 30 minutes the solution was diluted with DMSO and purified by C18 ISCO RP-HPLC. Upon lyophilization 4-((S)-2-((S)-2-((tert-butoxycarbonyl)amino)-3-methylbutanamido)-5-ureidopentanamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (133 mg, 63%) was obtained as the formic acid salt. HRMS: MH⁺=984.4800, Rt=1.85 min (5 min acidic method).

### Synthesis of 1-(5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

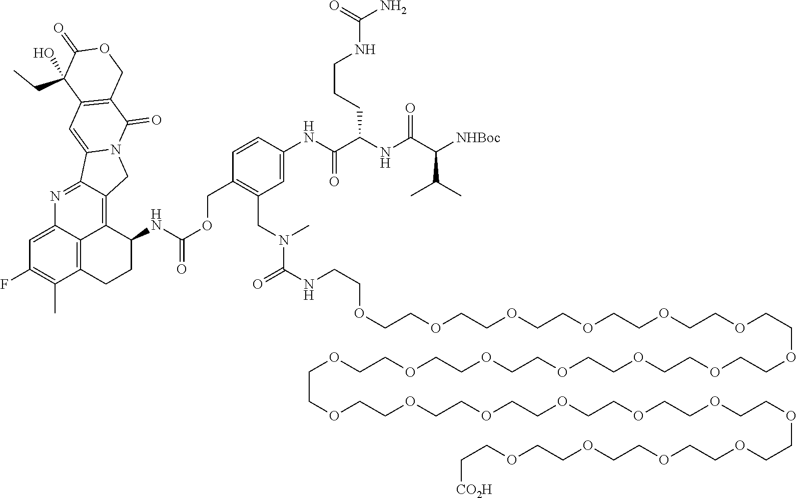 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)OC(C)(C)C)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3]

To a solution of bis(4-nitrophenyl) carbonate (105.8 mg, 347.9 μmol) and 1-amino-3,6,9,12,15,18,21,24,27,30,33,36,39,42,45,48,51,54,57,60,63,66,69,72-tetracosaoxapentaheptacontan-75-oic acid (415.5 mg, 362.4 μmol) in DMF (4 mL) was added DIEA (227 μL, 1305 μmol). After stirring for 1.5 hours, 4-((S)-2-((tert-butoxycarbonyl)amino)-5-ureidopentanamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate formic acid salt (318.4 mg, 290 μmol) and DIEA (227 μL, 1305 μmol) were added. After stirring an additional 2 hours, the solution was diluted with DMSO and purified by RP-HPLC. Upon lyophilization, 1-(5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (466 mg, 74% yield) was obtained. HRMS: (M⁺2Na⁺-H⁺)⁺=2156.1499, Rt=2.36 min (5 min acidic method).

### Synthesis of 1-(5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

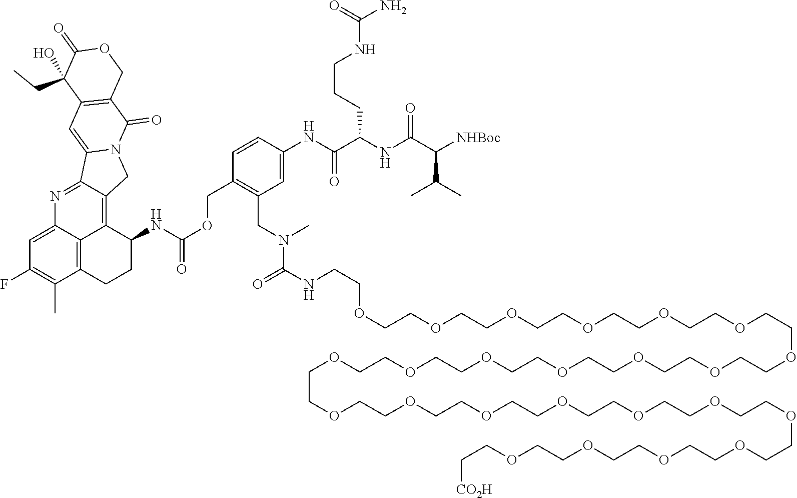 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)OC(C)(C)C)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3]

1-(5-((S)-2-((S)-2-((tert-butoxycarbonyl)amino)-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (195 mg, 90.4 μmol) was treated with 25% TFA/CH2Cl2 (6 mL) for 1 hour at which the volatiles were removed in vacuo. The residue was dissolved in DMSO (12 mL) and purified by ISCO gold RP-HPLC. Upon lyophilization, 1-(5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid TFA salt (190 mg, 96% yield) was obtained. HRMS: MH⁺=2056.0801, Rt=1.89 min (5 min acidic method).

### Synthesis of 1,1′-((((2S,5S,39S,42S)-22-(3-(2-((((9H-fluoren-9-yl)methoxy)carbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(6-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3,1-phenylene))bis(2-methyl-3-oxo-7′,10′,13′,16′,19′,22′,25′,28′,31′,34′,37′,40′,43′,46′,49′,52′,55′,58′,61′,64′,67′,70′,73′,76′-tetracosaoxa-2′,4′-diazanonaheptacontan-79-oic acid)

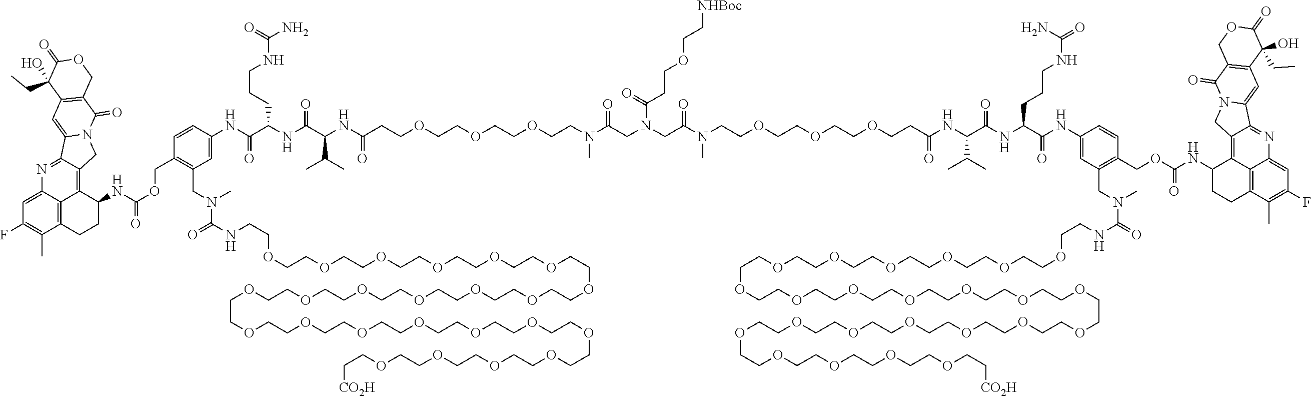

To a solution of 1-(5-((S)-2-((S)-2-amino-3-methylbutanamido)-5-ureidopentanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid trifluoroacetate salt (353 mg, 162.6 μmol) and bis(2,5-dioxopyrrolidin-1-yl) 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate (75.1 mg, 68.3 μmol) in NMP (3 mL) was added DIEA (56.7 uL, 353 μmol). After 4 hours the solution was diluted with DMSO and purified by ISCO RP-HPLC. Upon lyophilization, 1,1′-((((2S,5S,39S,42S)-22-(3-(2-((((9H-fluoren-9-yl)methoxy)carbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(6-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3,1-phenylene))bis(2-methyl-3-oxo-7′,10′,13′,16′,19′,22′,25′,28′,31′,34′,37′,40′,43′,46′,49′,52′,55′,58′,61′,64′,67′,70′,73′,76′-tetracosaoxa-2′,4′-diazanonaheptacontan-79-oic acid) (180 mg, 47% yield) was obtained. HRMS: MH⁺=4979.5601, Rt=2.56 min (5 min acidic method).

### Synthesis of (6S,9S)-1-amino-26-((6S,9S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9-isopropyl-23-methyl-1,8,11,24-tetraoxo-14,17,20-trioxa-2,7,10,23-tetraazapentacosan-25-yl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9-isopropyl-23-methyl-1,8,11,24,27-pentaoxo-14,17,20,30-tetraoxa-2,7,10,23,26-pentaazadotriacontan-32-aminium formate

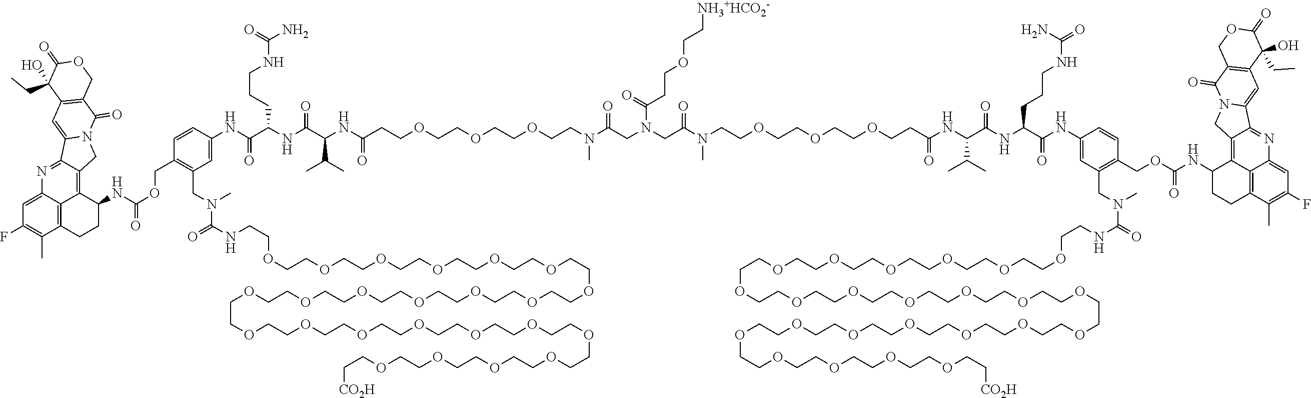

A solution of 1,1′-((((2S,5S,39S,42S)-22-(3-(2-((((9H-fluoren-9-yl)methoxy)carbonyl)amino)ethoxy)propanoyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(6-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3,1-phenylene))bis(2-methyl-3-oxo-7′,10′,13′,16′,19′,22′,25′,28′,31′,34′,37′,40′,43′,46′,49′,52′,55′,58′,61′,64′,67′,70′,73′,76′-tetracosaoxa-2′,4′-diazanonaheptacontan-79-oic acid) (180 mg, 36.1 μmol) in DMF was treated with piperidine (35.7 uL, 361 μmol) for 1 hour at which time the solution was diluted with DMSO and purified by ISCO RP-HPLC. Upon lyophilization, (6S,9S)-1-amino-26-((6S,9S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9-isopropyl-23-methyl-1,8,11,24-tetraoxo-14,17,20-trioxa-2,7,10,23-tetraazapentacosan-25-yl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9-isopropyl-23-methyl-1,8,11,24,27-pentaoxo-14,17,20,30-tetraoxa-2,7,10,23,26-pentaazadotriacontan-32-aminium formate (75 mg, 41% yield) was obtained. HRMS: MH⁺=4757.4902, Rt=2.16 min (5 min acidic method).

### Example #7: Synthesis of 1,1′-((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(6-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3,1-phenylene))bis(2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid), P7-L12-P7

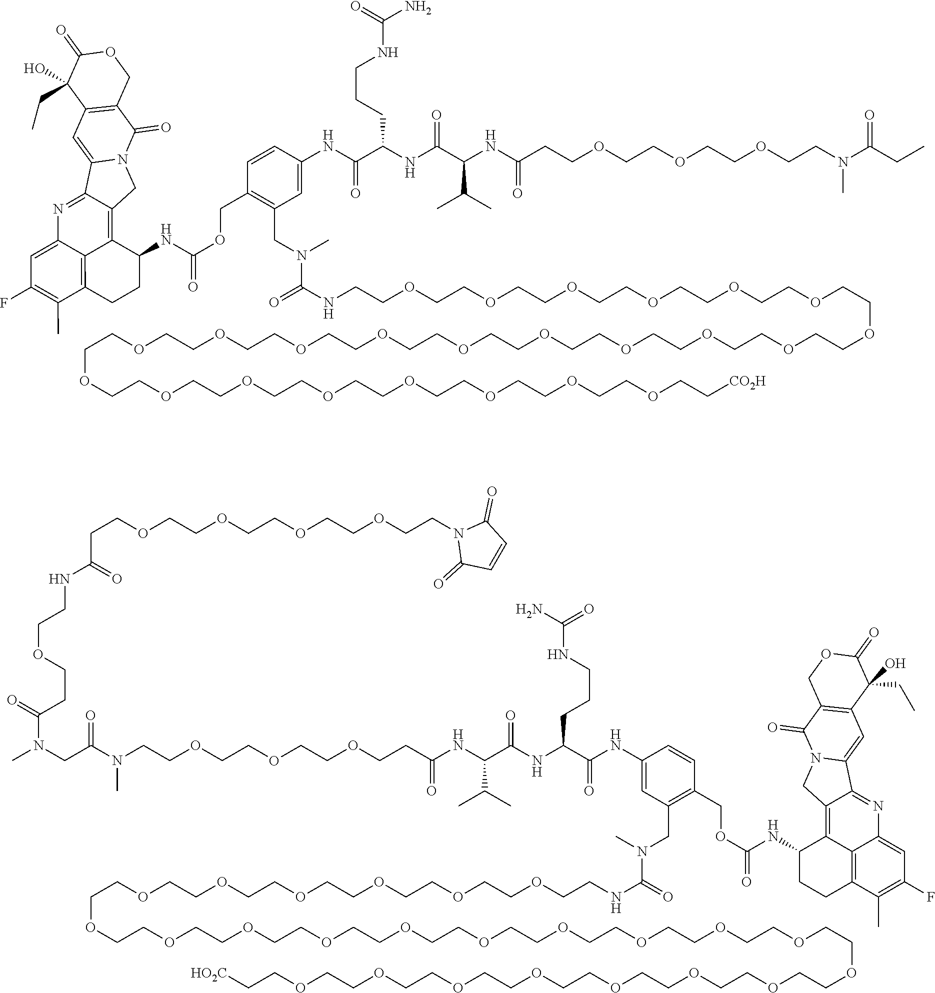

To a solution of (6S,9S)-1-amino-26-((6S,9S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9-isopropyl-23-methyl-1,8,11,24-tetraoxo-14,17,20-trioxa-2,7,10,23-tetraazapentacosan-25-yl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenyl)carbamoyl)-9-isopropyl-23-methyl-1,8,11,24,27-pentaoxo-14,17,20,30-tetraoxa-2,7,10,23,26-pentaazadotriacontan-32-aminium formate (130 mg, 27.05 μmol), 2,5-dioxopyrrolidin-1-yl 1-(2,5-dioxo-2,5-dihydro-1H-pyrrol-1-yl)-3,6,9,12-tetraoxapentadecan-15-oate (19.2 mg, 43.3 μmol) in DMF was added DIEA (23.6 μL, 135.3 μmol). After standing for 3 hours the solution was diluted with DMSO and purified by RP-HPLC. Upon lyophilization, 1,1′-((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(6-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-3,1-phenylene))bis(2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid) (42 mg, 29% yield) was obtained. HRMS: MH⁺=5084.6401, Rt=2.36 min (5 min acidic method).

### Synthesis of di-tert-butyl (2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl) phosphate

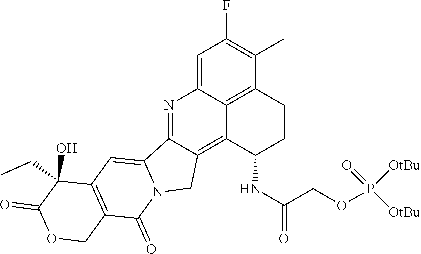 [SMILES: CC[C@@]1(O)C(=O)OCc2c1cc1n(c2=O)Cc2c-1nc1cc(F)c(C)c3c1c2[C@@H](NC(=O)COP(=O)(OC(C)(C)C)OC(C)(C)C)CC3]

To a mixture of (1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-aminium methanesulfonate (420 mg, 790.2 μmol) and 2-((di-tert-butoxyphosphoryl)oxy)acetic acid (530 mg, 1976 μmol) in DMF (12 mL) was added TSTU (594.6 mg, 1976 μmol) and DIEA (1.39 mL, 7900 mmol). After stirring for 2 hours the solution was diluted with DMSO and purified by ISCO RP-HPLC. Upon lyophilization, di-tert-butyl (2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl) phosphate (308.3 mg, 57% yield) was obtained. LC/MS: (M-tBu-tBu+H⁺)⁺=574.4, Rt=1.05 min (2 min acidic method).

### Synthesis of 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl dihydrogen phosphate

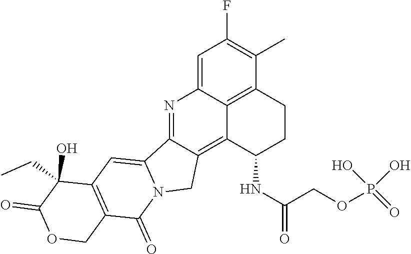 [SMILES: CC[C@@]1(O)C(=O)OCc2c1cc1n(c2=O)Cc2c-1nc1cc(F)c(C)c3c1c2[C@@H](NC(=O)COP(=O)(O)O)CC3]

di-tert-butyl (2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl) phosphate (308.3 mg, 449.6 μmol) was treated 25% TFA/CH2Cl2 (6 mL) at 0 C for 15 minutes and then at rt for 30 minutes at which time the volatiles were removed in vacuo. The residue was dissolved in DMSO and purified by ISCO RP-HPLC. Upon lyophilization, 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl dihydrogen phosphate (188 mg, 73% yield) was obtained. LC/MS: MH⁺=574.4, Rt=0.53 min (2 min acidic method).

### Synthesis of allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-((methylamino)methyl)phenyl)amino)-1-oxo-5-ureidopentan-2-yl)amino)-3-methyl-1-oxobutan-2-yl)carbamate

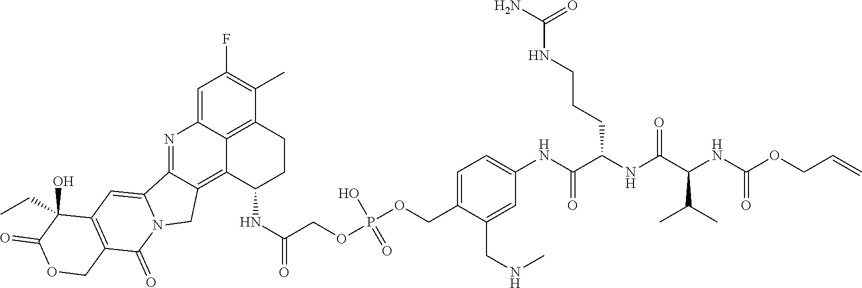 [SMILES: C=CCOC(=O)N[C@H](C(=O)N[C@@H](CCCNC(N)=O)C(=O)Nc1ccc(COP(=O)(O)OCC(=O)N[C@H]2CCc3c(C)c(F)cc4nc5c(c2c34)Cn2c-5cc3c(c2=O)COC(=O)[C@]3(O)CC)c(CNC)c1)C(C)C]

A solution of 2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl dihydrogen phosphate (256 mg, 446.4 μmol) in DMF (1.5 mL) and N,N′-dicyclohexylmorpholine-4-carboximidamide (393 mg, 1339 μmol) was stirred for 15 minutes at which time (9H-fluoren-9-yl)methyl (5-((S)-2-((S)-2-(((allyloxy)carbonyl)amino)-3-methylbutanamido)-5-ureidopentanamido)-2-(chloromethyl)benzyl)(methyl)carbamate (500 mg, 670 μmol) was added. After stirring for 20 hours Et2NH (924 uL, 8928 μmol) was added. After stirring an additional 30 minutes the solution was diluted with DMSO and purified by ISCO RP-HPLC. Upon lyophilization, allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-((methylamino)methyl)phenyl)amino)-1-oxo-5-ureidopentan-2-yl)amino)-3-methyl-1-oxobutan-2-yl)carbamate (158 mg, 33% yield) was obtained as the formic acid salt. HRMS: MH⁺=1062.4000, Rt=1.75 min (5 min acidic method).

### Synthesis of 1-(5-((S)-2-((S)-2-(((allyloxy)carbonyl)amino)-3-methylbutanamido)-5-ureidopentanamido)-2-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

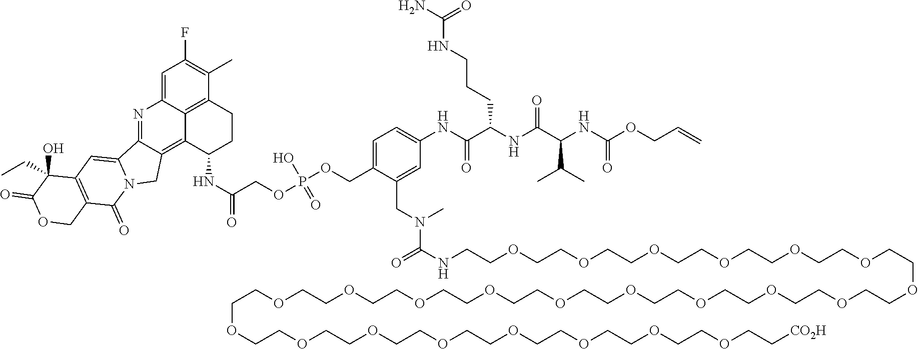 [SMILES: C=CCOC(=O)N[C@H](C(=O)N[C@@H](CCCNC(N)=O)C(=O)Nc1ccc(COP(=O)(O)OCC(=O)N[C@H]2CCc3c(C)c(F)cc4nc5c(c2c34)Cn2c-5cc3c(c2=O)COC(=O)[C@]3(O)CC)c(CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)c1)C(C)C]

To a solution of bis(4-nitrophenyl) carbonate (71.6 mg, 235.2 μmol) and 1-amino-3,6,9,12,15,18,21,24,27,30,33,36,39,42,45,48,51,54,57,60,63,66,69,72-tetracosaoxapentaheptacontan-75-oic acid (269.7 mg, 235.2 μmol) in DMF (2 mL) was added DIEA (205 μL, 1176 μmol). After stirring for 1 hour, allyl ((2S)-1-(((2S)-1-((4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3-((methylamino)methyl)phenyl)amino)-1-oxo-5-ureidopentan-2-yl)amino)-3-methyl-1-oxobutan-2-yl)carbamate formic acid salt (208.2 mg, 196 μmol) in DMF (2 mL) and DIEA (205 μL, 1176 μmol) were added. After stirring an additional 1 hour, the solution was diluted with DMSO and purified by RP-HPLC. Upon lyophilization, 1-(5-((S)-2-((S)-2-(((allyloxy)carbonyl)amino)-3-methylbutanamido)-5-ureidopentanamido)-2-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (218.9 mg, 50% yield) was obtained. HRMS: MH⁺=2234.0701, Rt=2.34 min (5 min acidic method).

### Synthesis of 1-(5-((S)-2-((S)-2-amino-3-methylbutanamido)-5-ureidopentanamido)-2-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

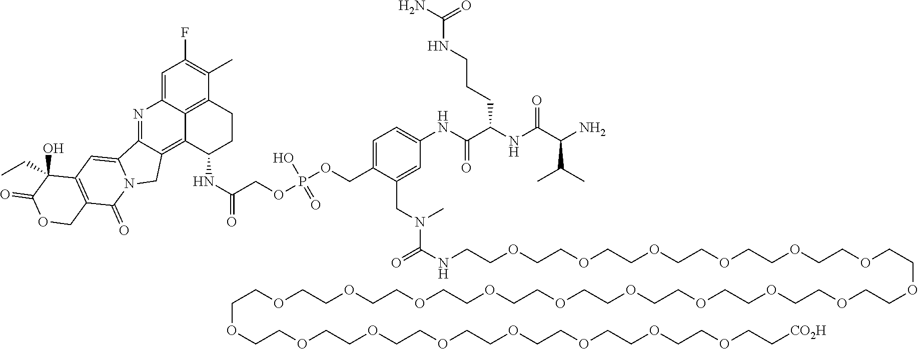 [SMILES: CC[C@@]1(O)C(=O)OCc2c1cc1n(c2=O)Cc2c-1nc1cc(F)c(C)c3c1c2[C@@H](NC(=O)COP(=O)(O)OCc1ccc(NC(=O)[C@H](CCCNC(N)=O)NC(=O)[C@@H](N)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3]

To a solution 1-(5-((S)-2-((S)-2-(((allyloxy)carbonyl)amino)-3-methylbutanamido)-5-ureidopentanamido)-2-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (233 mg, 104.3 μmol) and phenyl silane (33.9 mg, 312.9 μmol) in 1:1 CH₂Cl₂/MeOH (2 mL) under N2 atmosphere was added tetrakis(triphenylphosphine)palladium (1.2 mg, 1.04 μmol). After stirring for 2 hours the volatiles were removed in vacuo, the residue was dissolved in DMSO and was purified by RP-HPLC. Upon lyophilization, 1-(5-((S)-2-((S)-2-amino-3-methylbutanamido)-5-ureidopentanamido)-2-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (161 mg, 71% yield) was obtained. HRMS: MH+=2150.0901, Rt=1.81 min (5 min acidic method).

### Synthesis of (6S,9S)-1-amino-26-((6S,9S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)carbamoyl)-9-isopropyl-23-methyl-1,8,11,24-tetraoxo-14,17,20-trioxa-2,7,10,23-tetraazapentacosan-25-yl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)carbamoyl)-9-isopropyl-23-methyl-1,8,11,24,27-pentaoxo-14,17,20,30-tetraoxa-2,7,10,23,26-pentaazadotriacontan-32-aminium trifluoroacetate

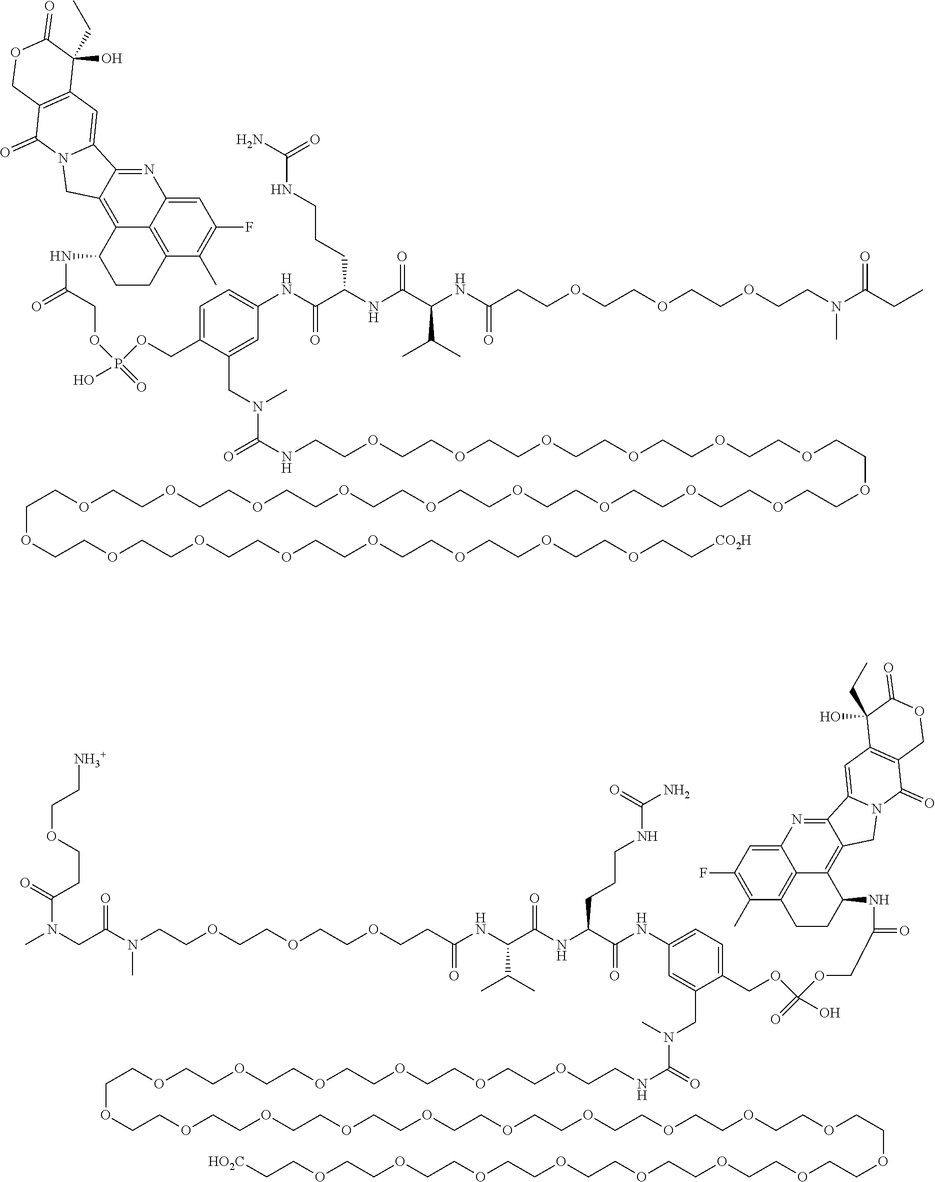

To a solution of 1-(5-((S)-2-((S)-2-amino-3-methylbutanamido)-5-ureidopentanamido)-2-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (159.6 mg, 74.2 μmol) and bis(2,5-dioxopyrrolidin-1-yl) 16-(3-(2-((((9H-fluoren-9-yl)methoxy)carbonyl)amino)ethoxy)propanoyl)-13,19-dimethyl-14,18-dioxo-4,7,10,22,25,28-hexaoxa-13,16,19-triazahentriacontanedioate (34.0 mg, 30.9 μmol) in DMF (1 mL) was added DIEA (53.9 uL, 309.3 μmol). After 4 hours Et2NH (64 uL, 618.7 μmol) was added and after 1 hour the solution was diluted with DMSO and purified by ISCO RP-HPLC. Upon lyophilization, (6S,9S)-1-amino-26-((6S,9S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)carbamoyl)-9-isopropyl-23-methyl-1,8,11,24-tetraoxo-14,17,20-trioxa-2,7,10,23-tetraazapentacosan-25-yl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)carbamoyl)-9-isopropyl-23-methyl-1,8,11,24,27-pentaoxo-14,17,20,30-tetraoxa-2,7,10,23,26-pentaazadotriacontan-32-aminium trifluoroacetate (108.6 mg, 70% yield) was obtained. HRMS: MH⁺=4945.4399, Rt=2.27 min (5 min acidic method).

### Example #8: Synthesis of 1,1′-((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(6-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3,1-phenylene))bis(2-methyl-3-oxo-7′,10′,13′,16′,19′,22′,25′,28′,31′,34′,37′,40′,43′,46′,49′,52′,55′,58′,61′,64′,67′,70′,73′,76′-tetracosaoxa-2′,4′-diazanonaheptacontan-79-oic acid), EX #8, P8-L12-P8

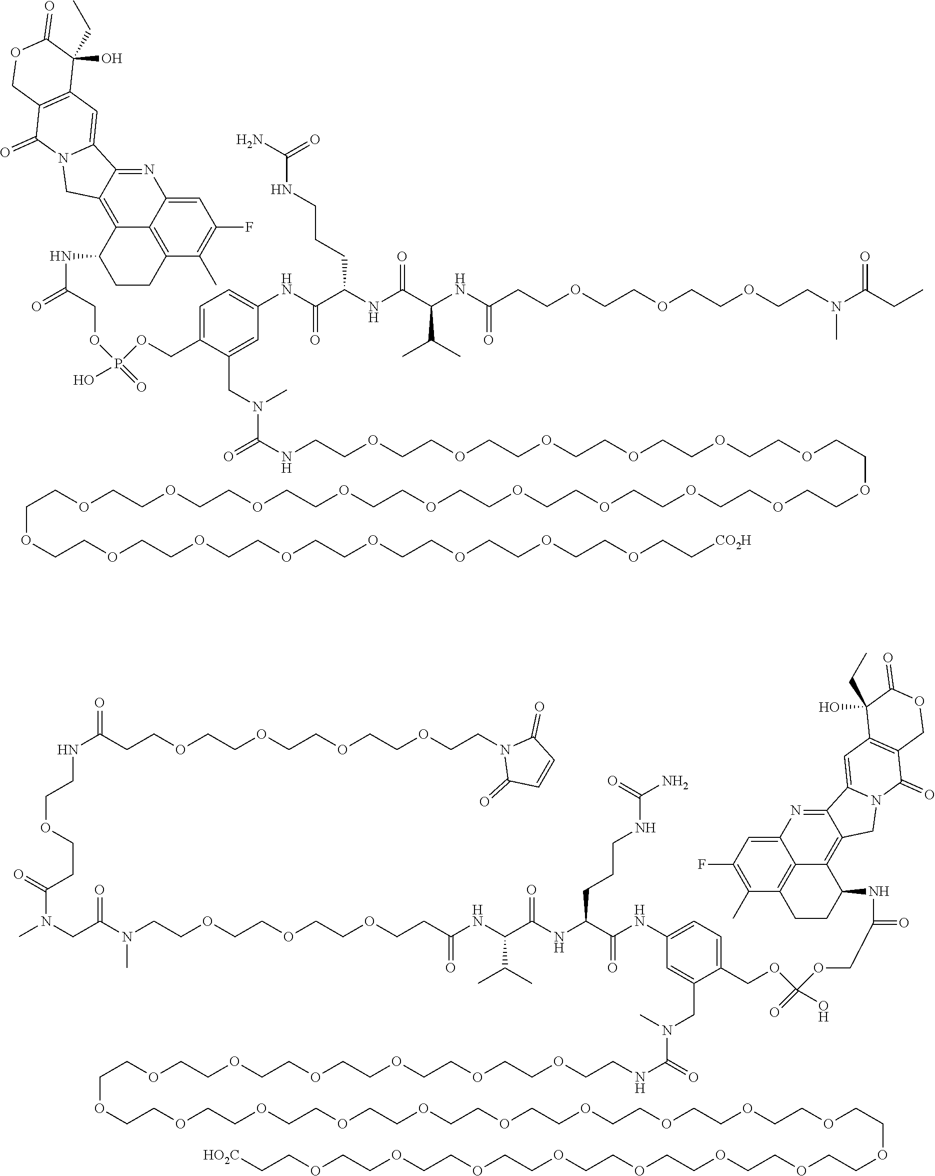

To a solution of (6S,9S)-1-amino-26-((6S,9S)-1-amino-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)carbamoyl)-9-isopropyl-23-methyl-1,8,11,24-tetraoxo-14,17,20-trioxa-2,7,10,23-tetraazapentacosan-25-yl)-6-((3-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)phenyl)carbamoyl)-9-isopropyl-23-methyl-1,8,11,24,27-pentaoxo-14,17,20,30-tetraoxa-2,7,10,23,26-pentaazadotriacontan-32-aminium trifluoroacetate (109 mg, 22 μmol) and 2,5-dioxopyrrolidin-1-yl 1-(2,5-dioxo-2,5-dihydro-1H-pyrrol-1-yl)-3,6,9,12-tetraoxapentadecan-15-oate (19.4 mg, 43.9 μmol) in DMF (0.6 mL) was added DIEA (57.4 μL, 329.3 μmol). After standing for 1 hour the solution was diluted with DMSO and purified by RP-HPLC. Upon lyophilization, 1,1′-((((2S,5S,39S,42S)-22-(1-(2,5-dioxo-2,5-dihydro-1H-pyrrol-1-yl)-15-oxo-3,6,9,12,19-pentaoxa-16-azadocosan-22-oyl)-5,39-diisopropyl-19,25-dimethyl-4,7,20,24,37,40-hexaoxo-2,42-bis(3-ureidopropyl)-10,13,16,28,31,34-hexaoxa-3,6,19,22,25,38,41-heptaazatritetracontanedioyl)bis(azanediyl))bis(6-((((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)(hydroxy)phosphoryl)oxy)methyl)-3,1-phenylene))bis(2-methyl-3-oxo-7′,10′,13′,16′,19′,22′,25′,28′,31′,34′,37′,40′,43′,46′,49′,52′,55′,58′,61′,64′,67′,70′,73′,76′-tetracosaoxa-2′,4′-diazanonaheptacontan-79-oic acid) (30.3 mg, 27% yield) was obtained. HRMS: MH⁺=5272.5601, Rt=2.70 min (5 min acidic method).

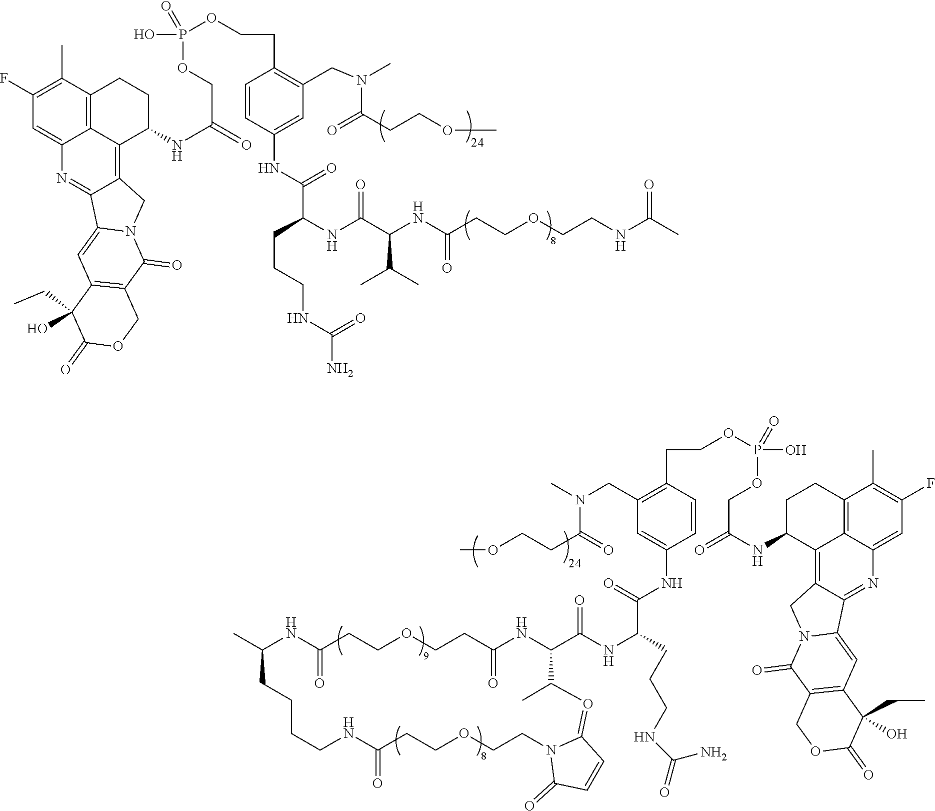

| | |
|---|---|
| Ex- ample #1 | 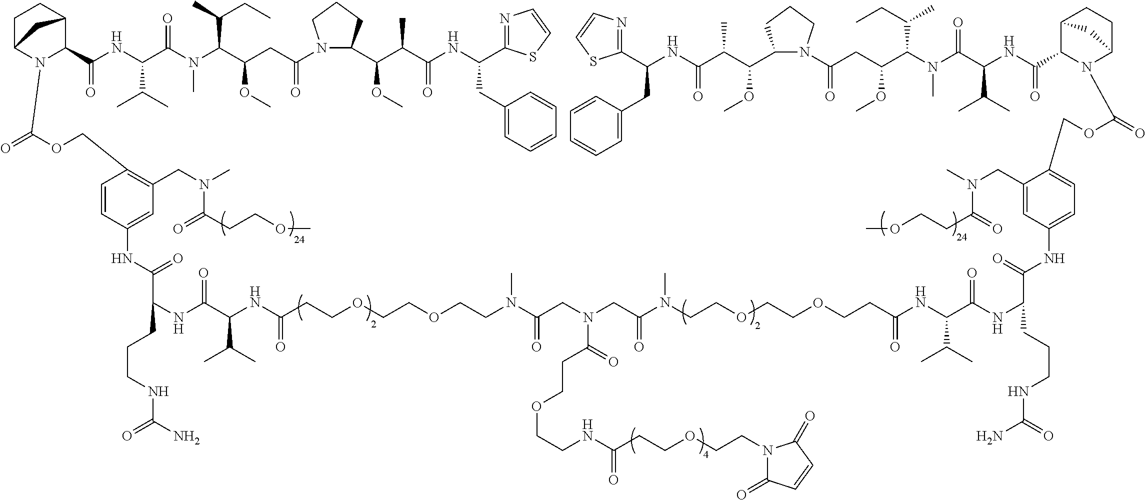 |
| Ex- ample #2 | 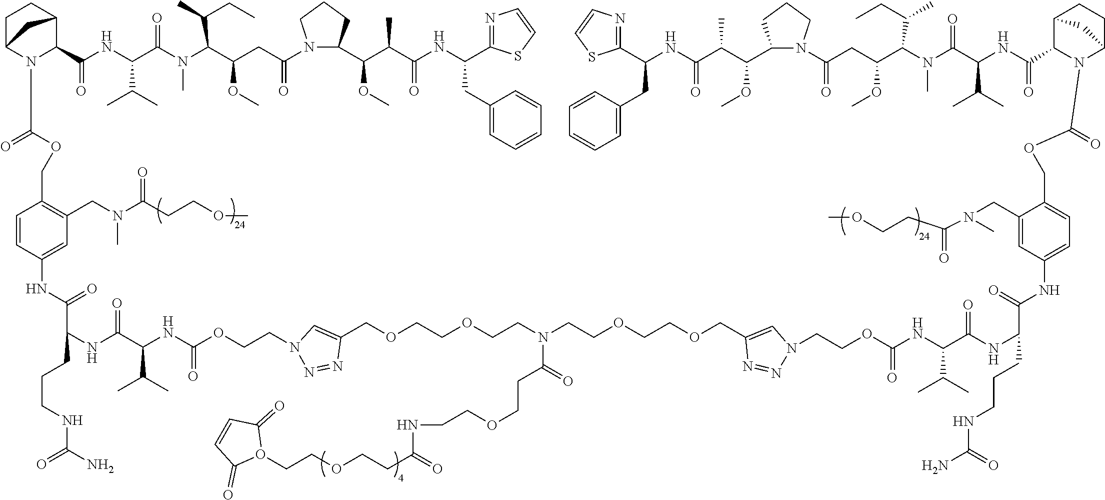 |
| Ex- ample #3 | 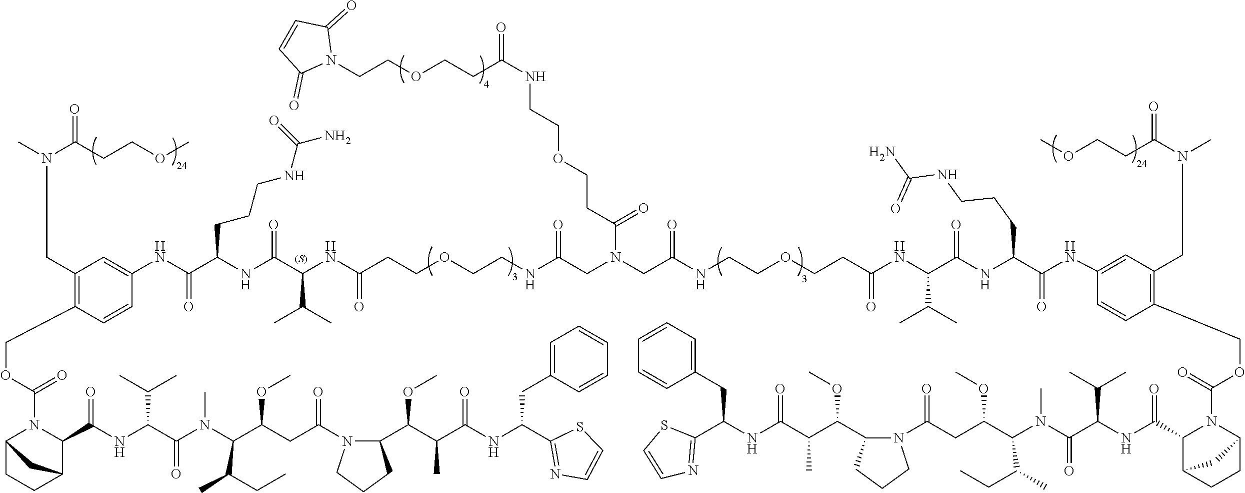 |
| Ex- ample #4 | 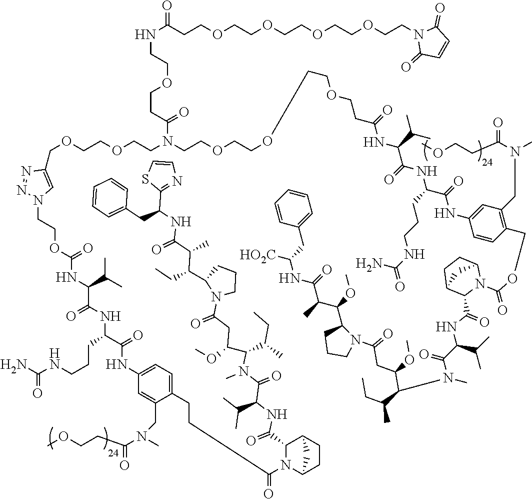 |
| Ex- ample #5 | 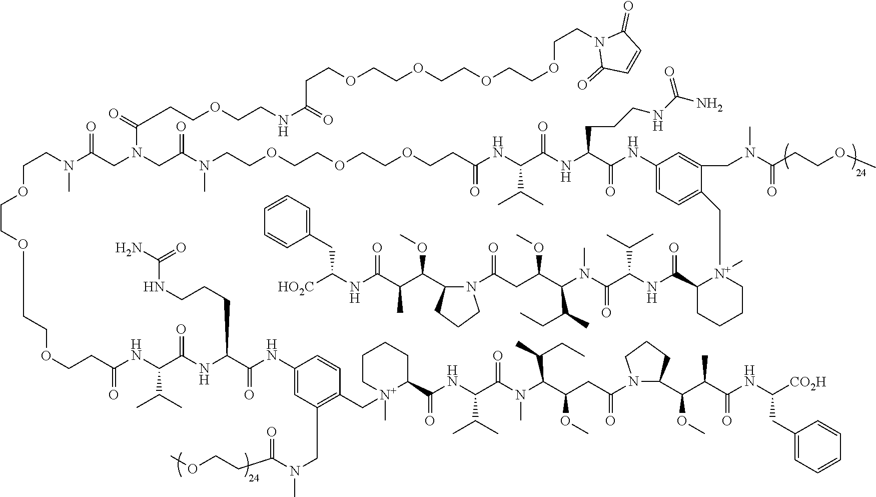 |
| Ex- ample #6 | 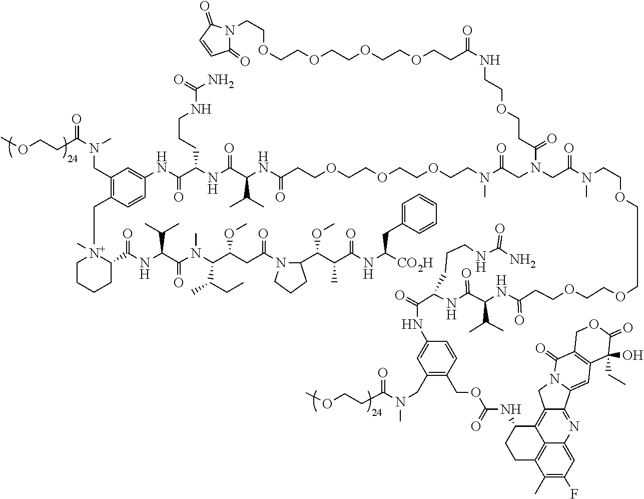 |
| Ex- ample #7 | 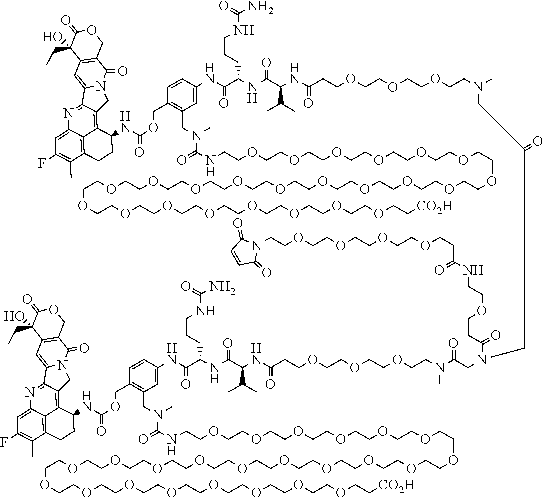 |
| Ex- ample #8 | 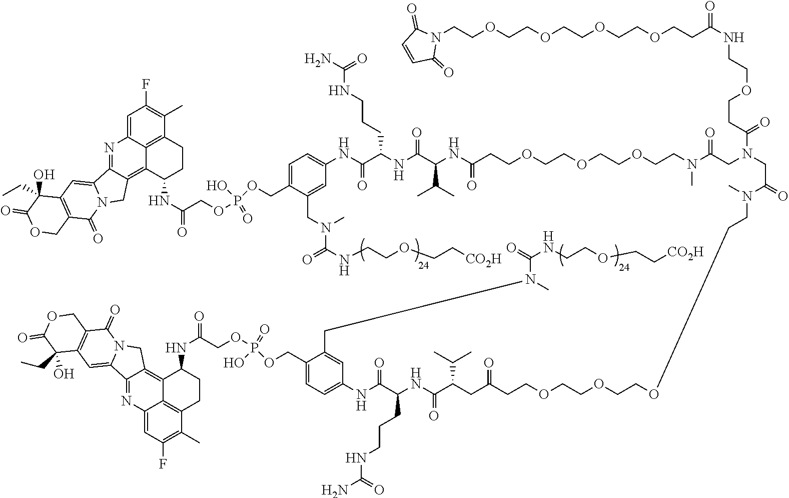 |
| P1 | In a manner similar to the preparation of Examples #1-8, the following compounds |
| | could be prepared (each P refers to the combination of the drug moiety D1 or D2, with |
| | A1 or A2 respectively and * in each P# of P1-P8 below represents the point of |
| | attachment to L, whilst L refers to the linker). As used herein, “P-L-P“ refers to the |
| | linker-payloads, linker-drugs, or linker-compounds disclosed herein and the terms “P#- |
| | L#-P#” refers to a specific linker-drug disclosed herein, wherein each of the codes “P#” |
| | refers to a specific drug unless otherwise specified and L# refers to a specific dual |
| | linker unless otherwise specified. The two “P#” codes can be the same or different, i.e. |
| | refers to the same or different drugs. For example, “P1-L1-P1” refers to the linker- |
| | payload compound with dual linker L1 attaches to two P1 payloads, while “P1-L1-P2” |
| | refers to the linker-payload compound with dual linker L1 attaches to a P1 and a P2 |
| | payload, including an enantiomer, diastereoisomer, atropisomer, deuterated |
| | derivative, and/or pharmaceutically acceptable salt of any of the foregoing. In some |
| | embodiments, for example, when the linker L1 is not symmetrical, the terms “P1-L1- |
| | P2” and “P2-L1-P1” refer to two different linker-drugs. |
| P2 | 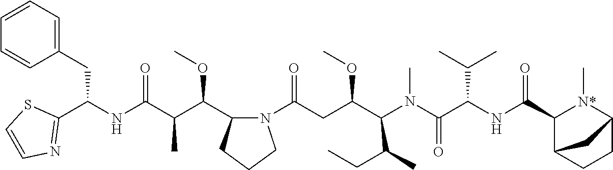 |
| | 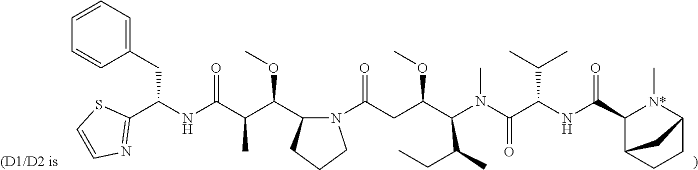 |
| P3 | 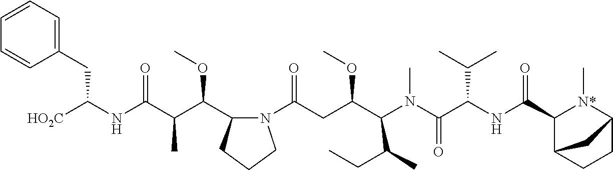 |
| | 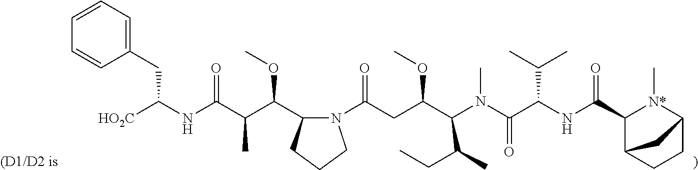 |
| P4 | 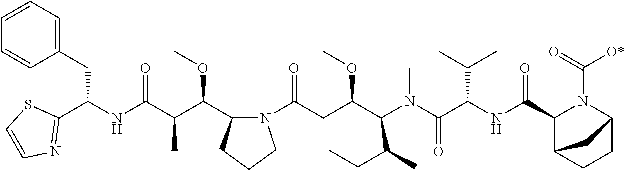 |
| | 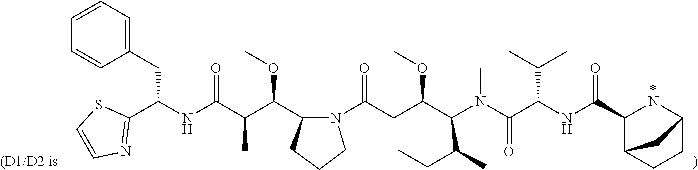 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H]1N[C@@H]2CC[C@H]1C2)C(C)C] |
| P5 | 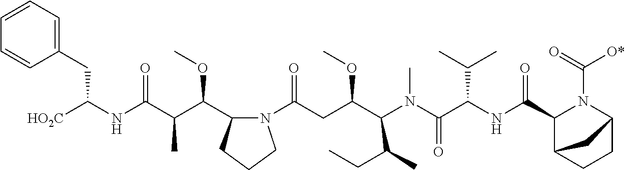 |
| | 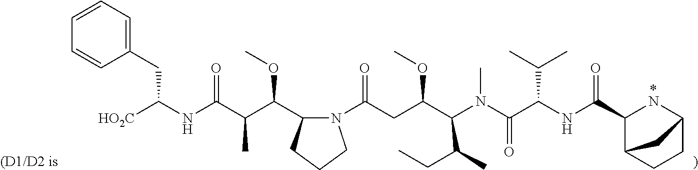 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H]1N[C@@H]2CC[C@H]1C2)C(C)C] |
| P6 | 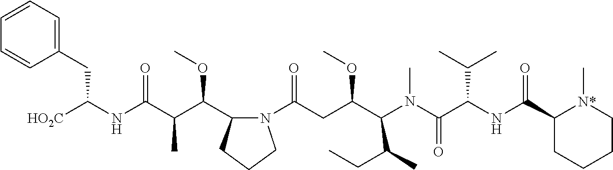 |
| | 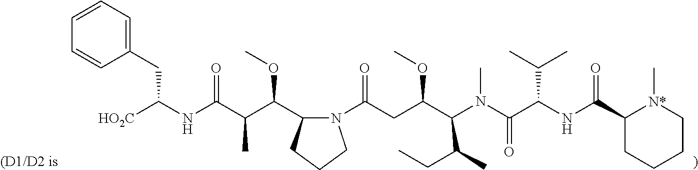 |
| P7 | 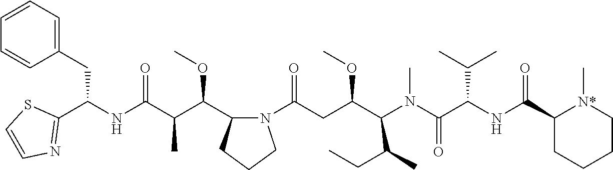 |
| | 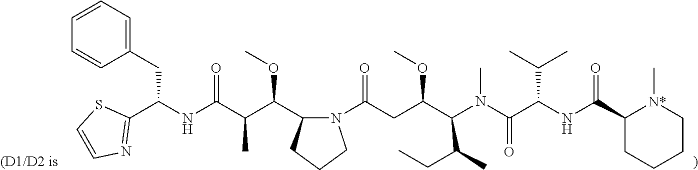 |
| P8 | 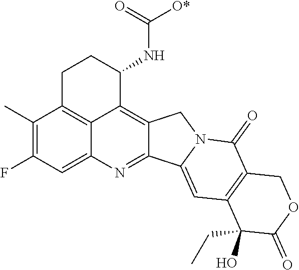 |
| | 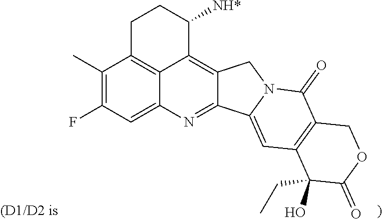 |
| P1-L1- P1 | 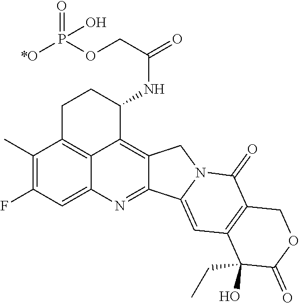 |
| | 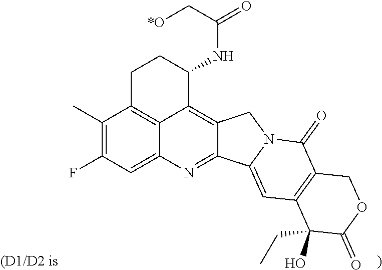 |
| P1-L1- P2 | 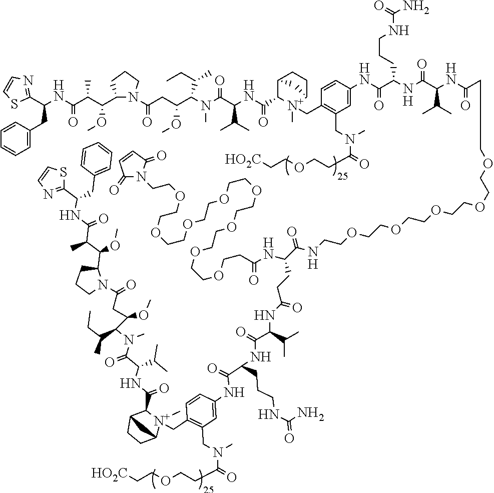 |
| P1-L1- P3 | 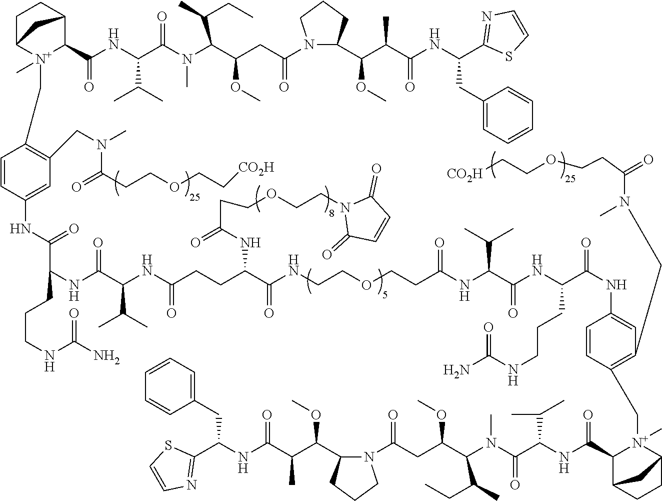 |
| P1-L1- P4 | 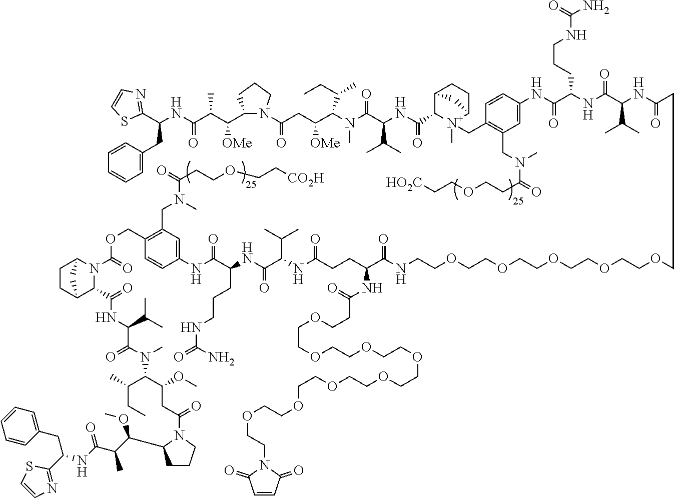 |
| P1-L1- P5 | 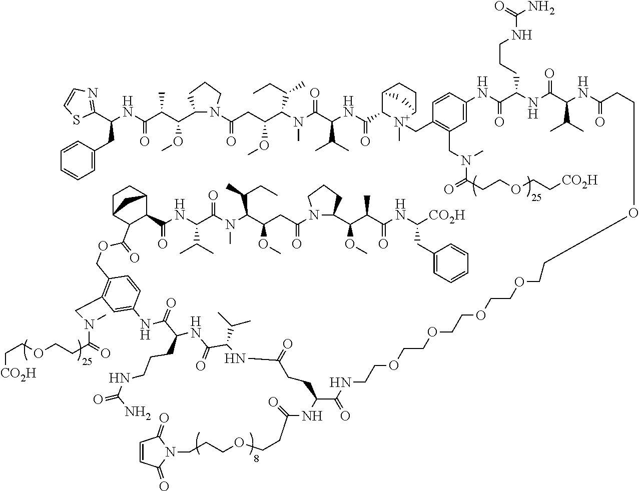 |
| P1-L1- P3 | 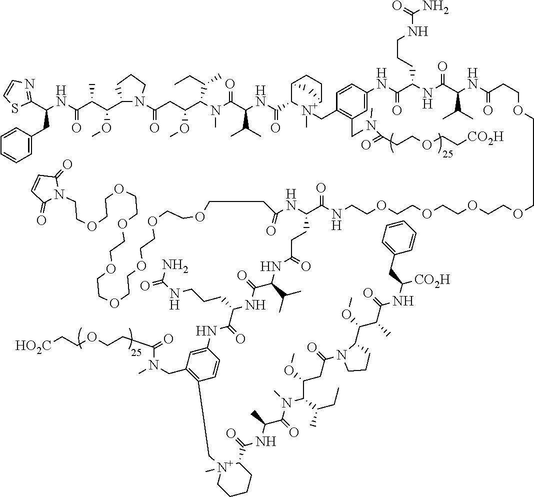 |
| P2-L1- P1 | 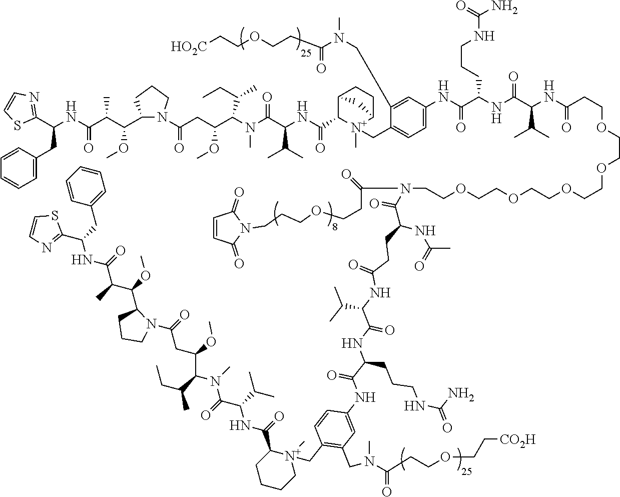 |
| P2-L1- P2 | 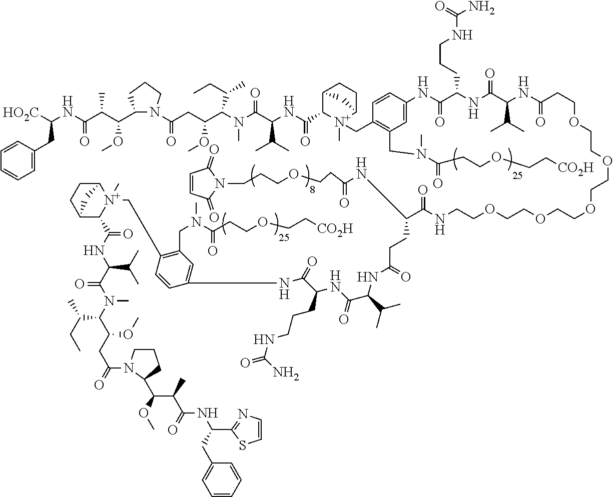 |
| P2-L1- P3 | 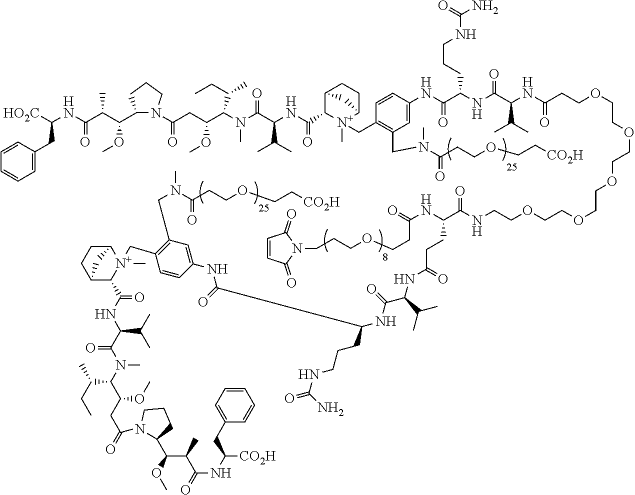 |
| P2-L1- P4 | 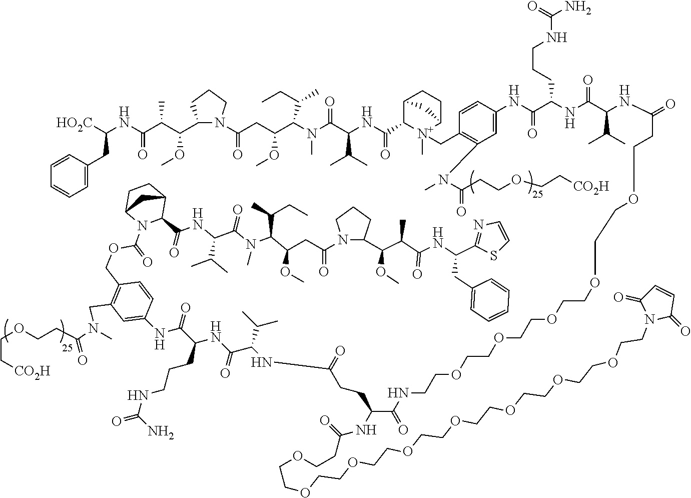 |
| P2-L1- P5 | 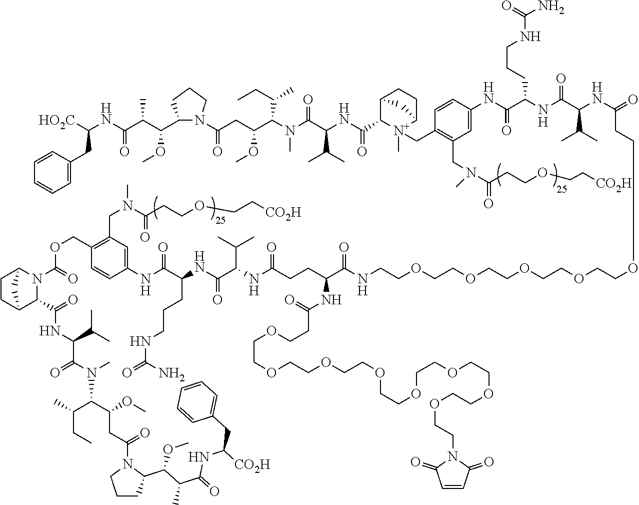 |
| P2-L1- P6 | 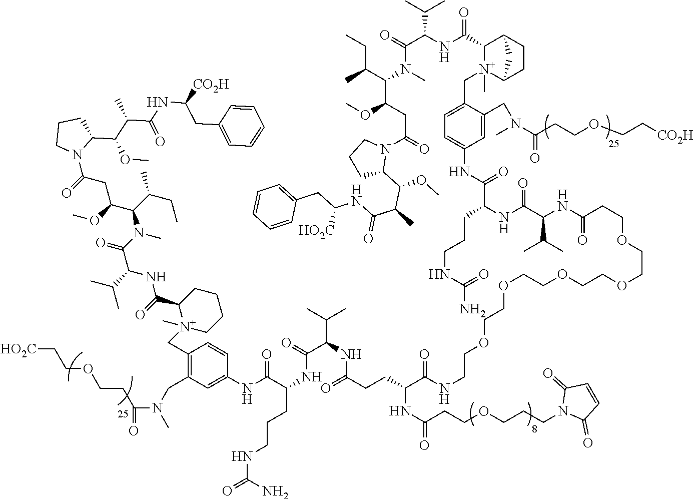 |
| P3-L1- P1 | 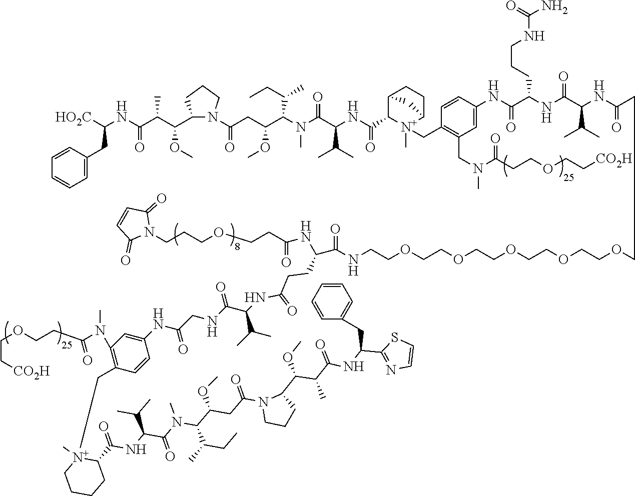 |
| P3-L1- P2 | 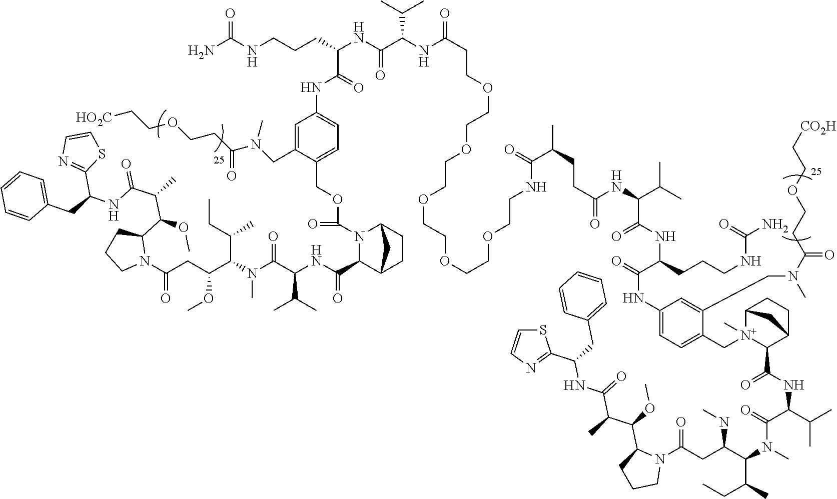 |
| P3-L1- P3 | 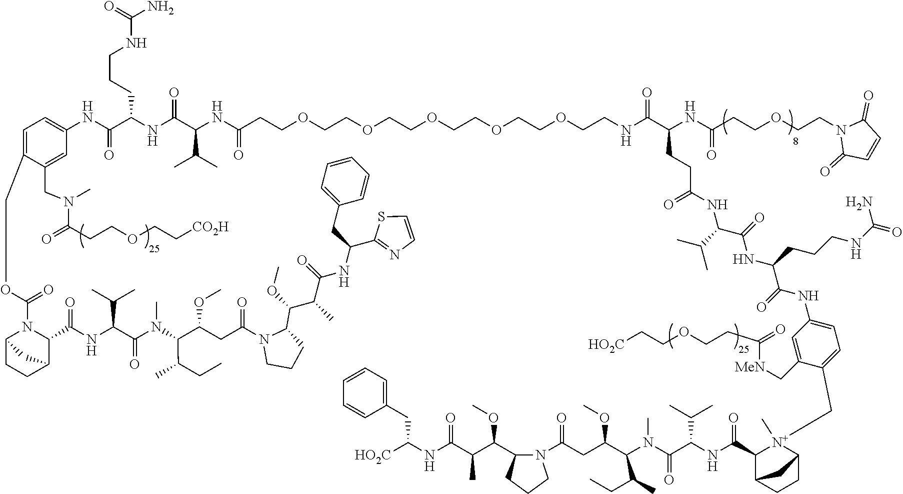 |
| P3-L1- P4 | 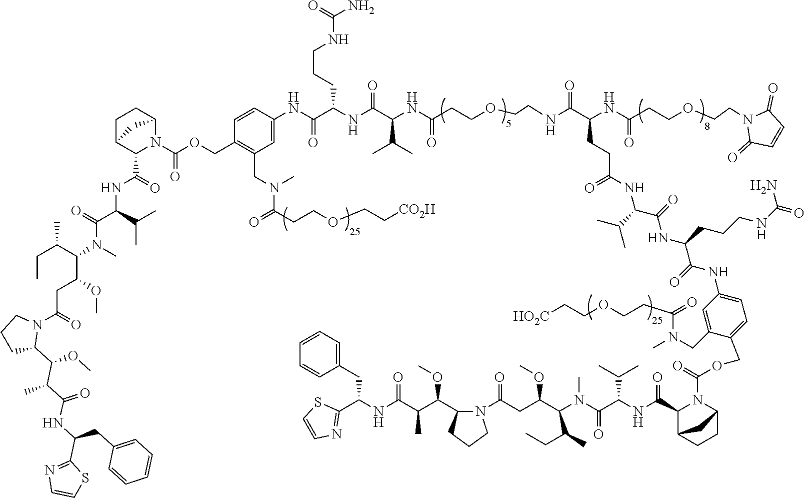 |
| P4-L1- P1 | 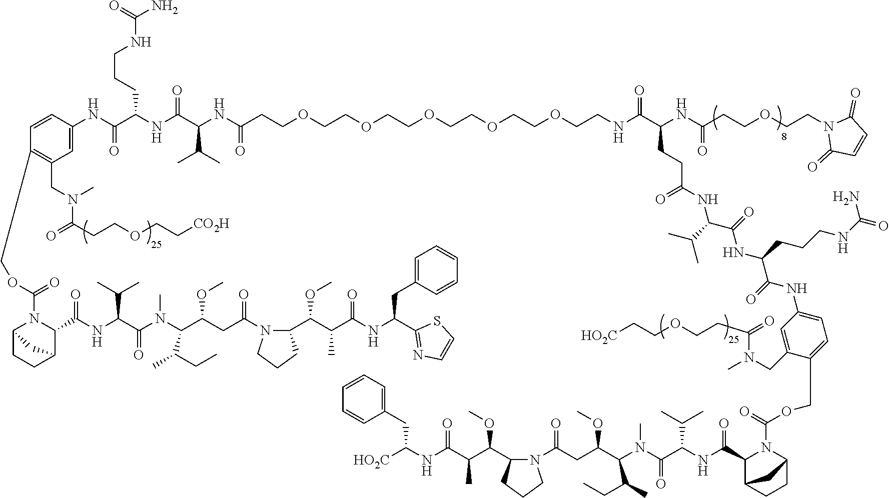 |
| P4-L1- P2 | 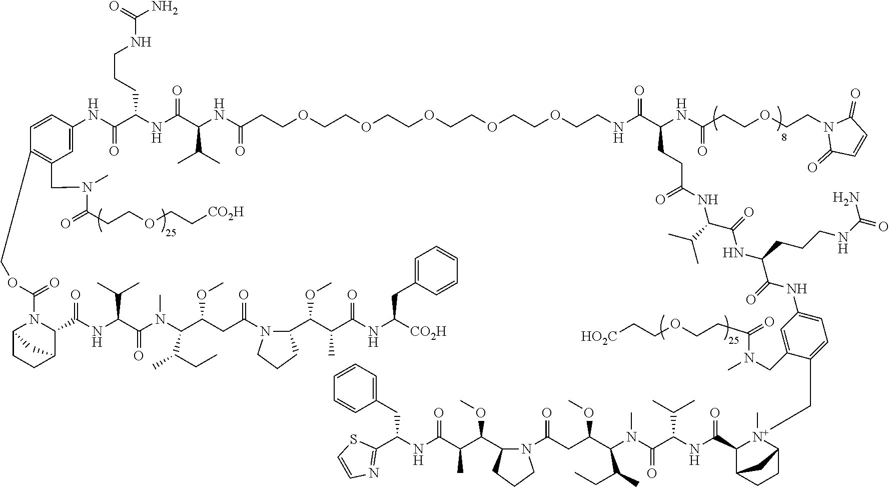 |
| P4-L1- P3 | 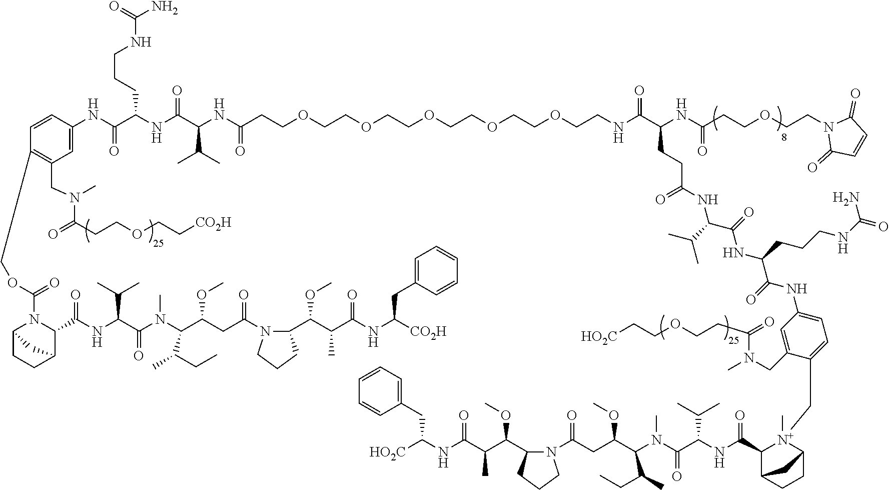 |
| P4-L1- P4 | 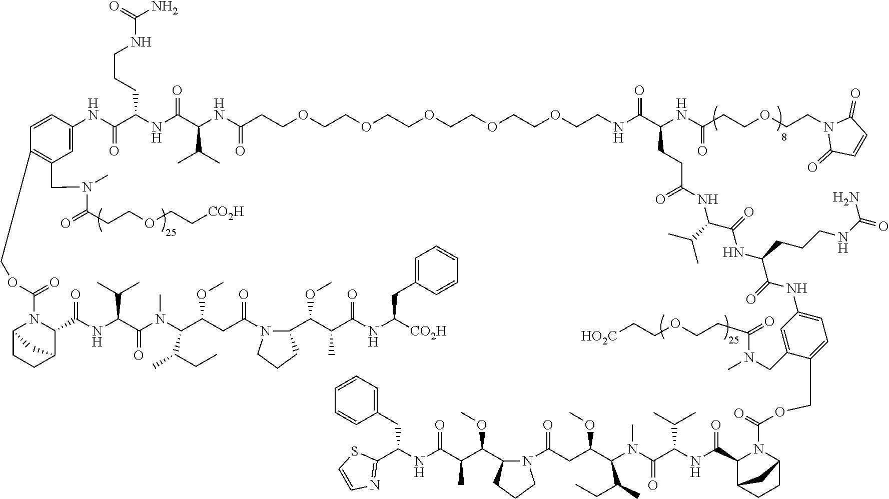 |
| P1-L2- P1 | 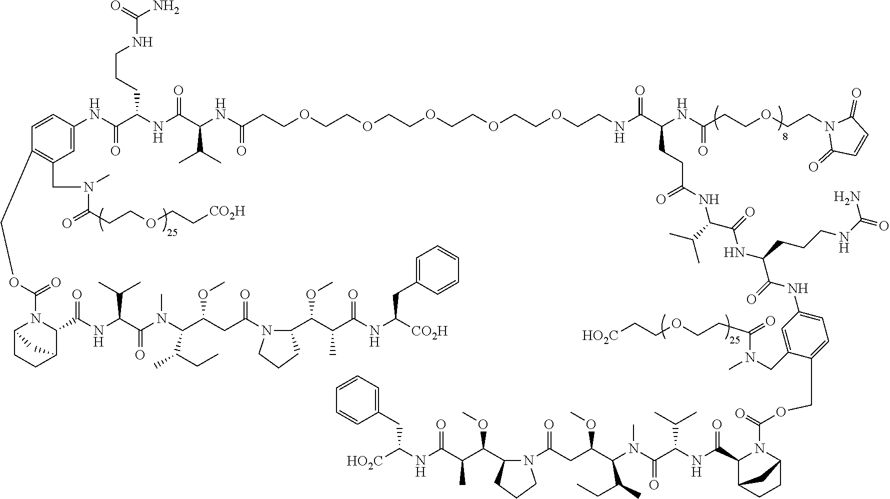 |
| P2-L2- P1 | 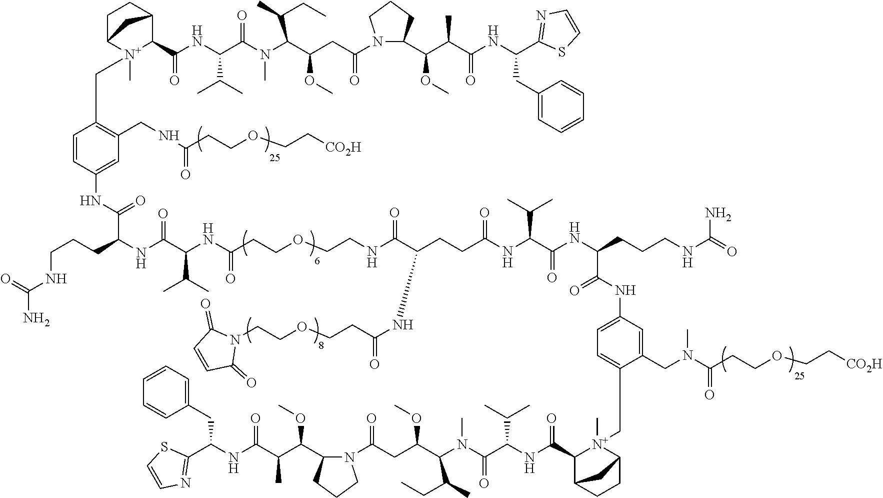 |
| P2-L2- P2 | 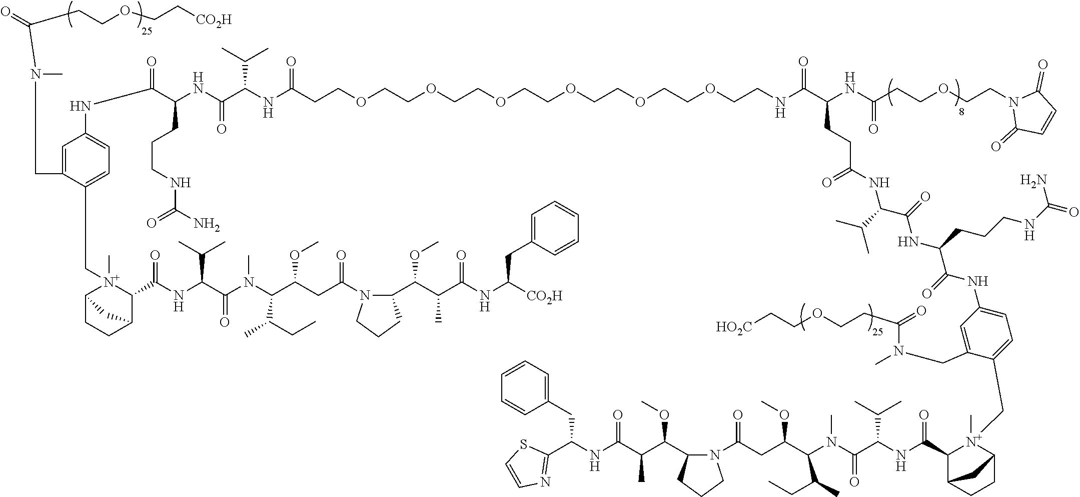 |
| P3-L2- P3 | 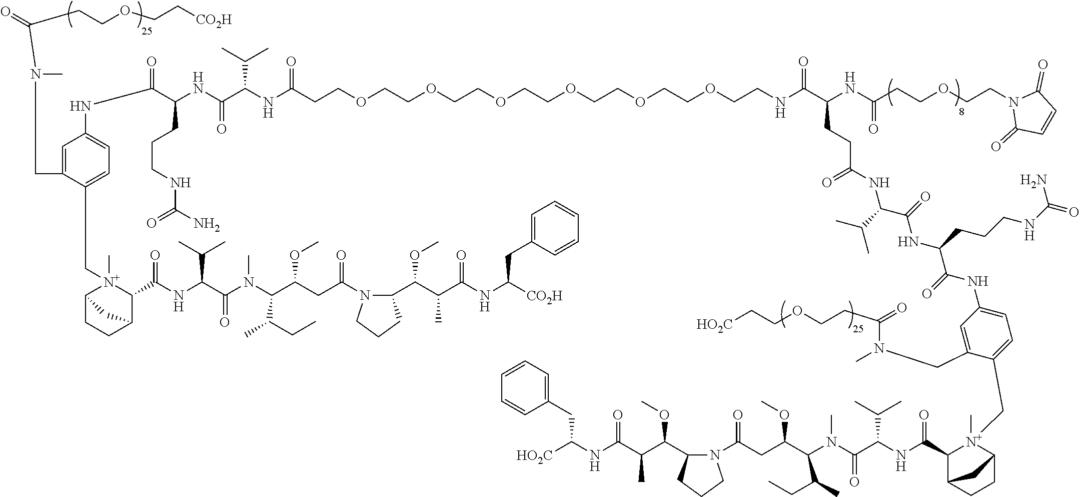 |
| P3-L2- P4 | 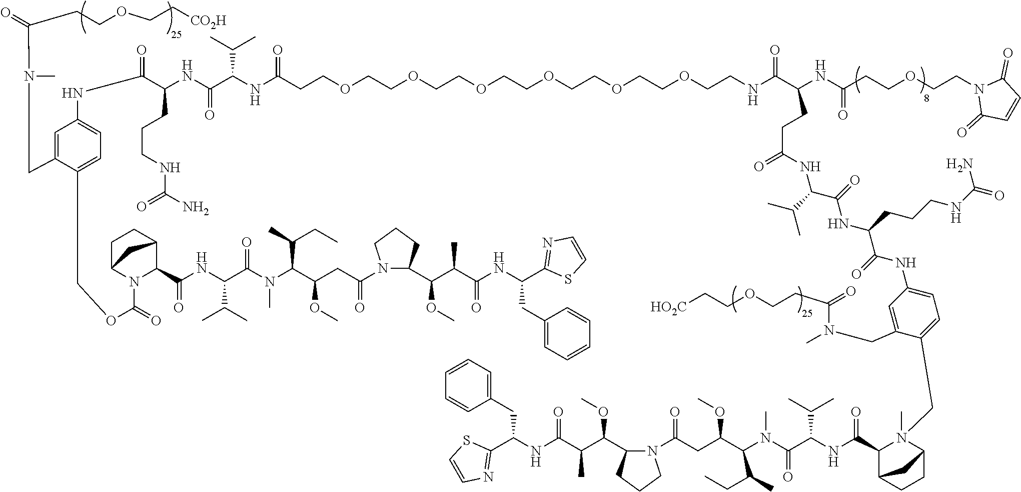 |
| P4-L2- P3 | 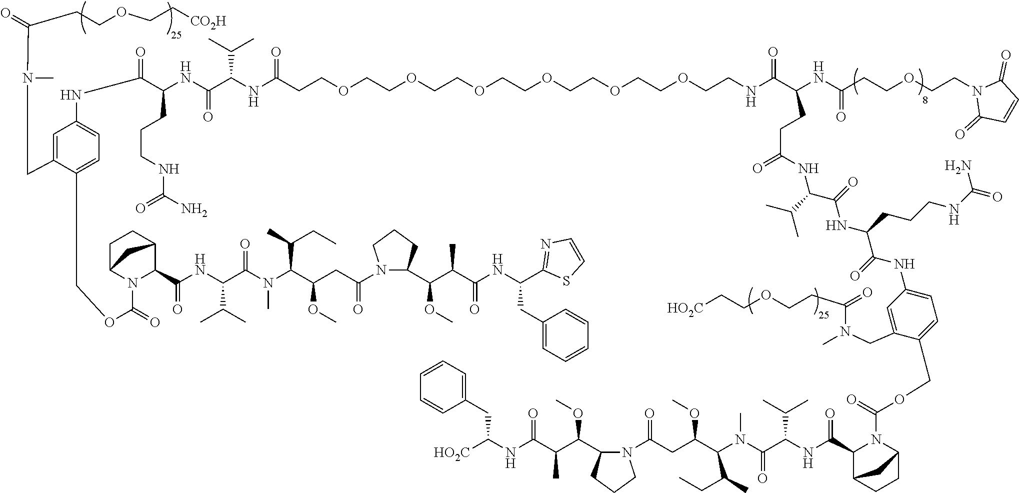 |
| P4-L2- P4 | 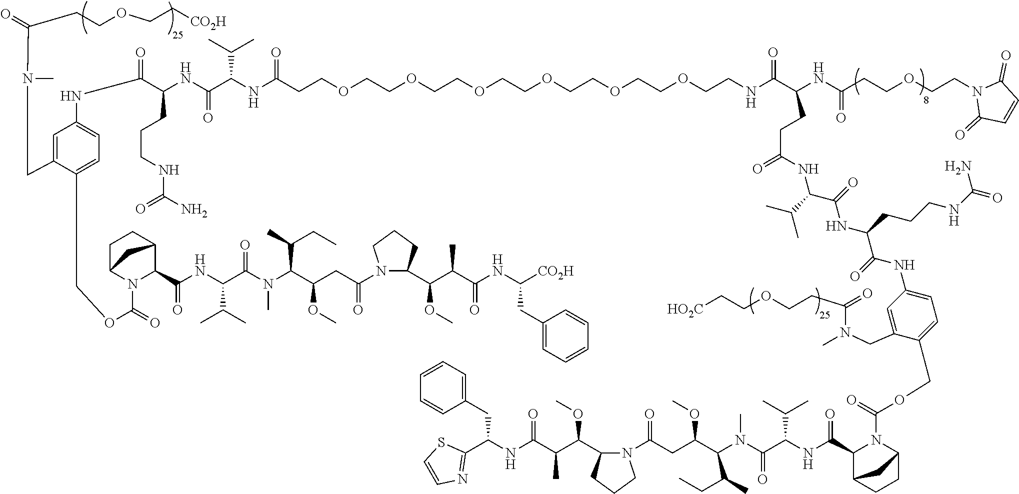 |
| P1-L3- P1 | 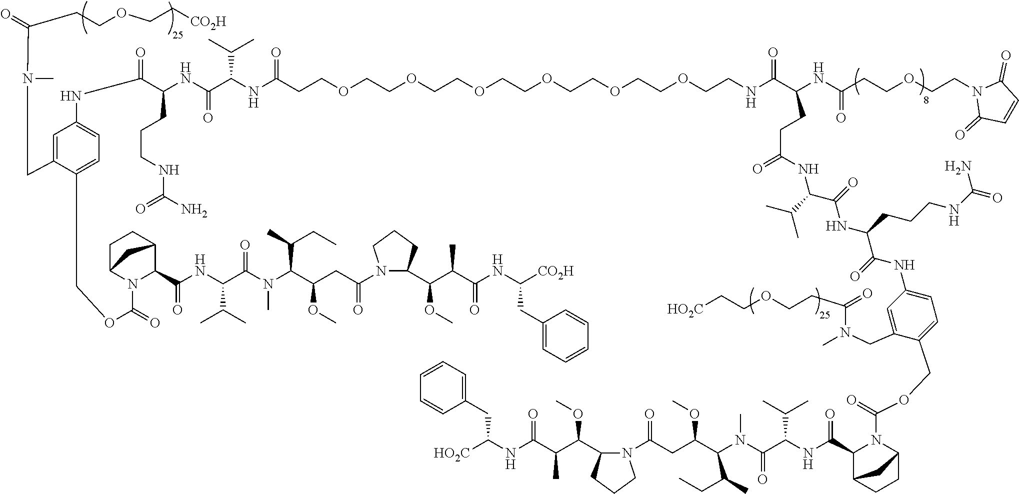 |
| P1-L3- P2 | 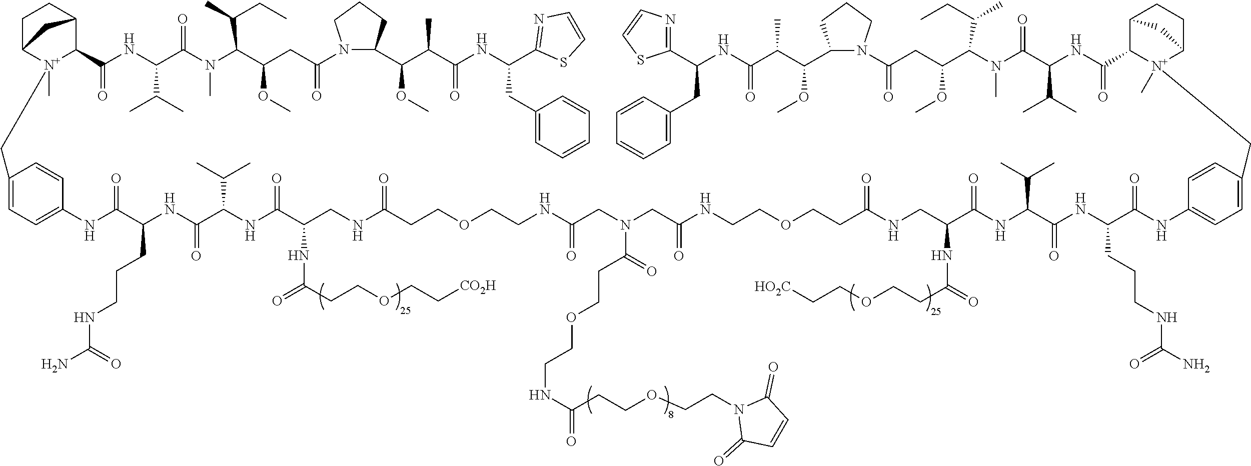 |
| P2-L3- P2 | 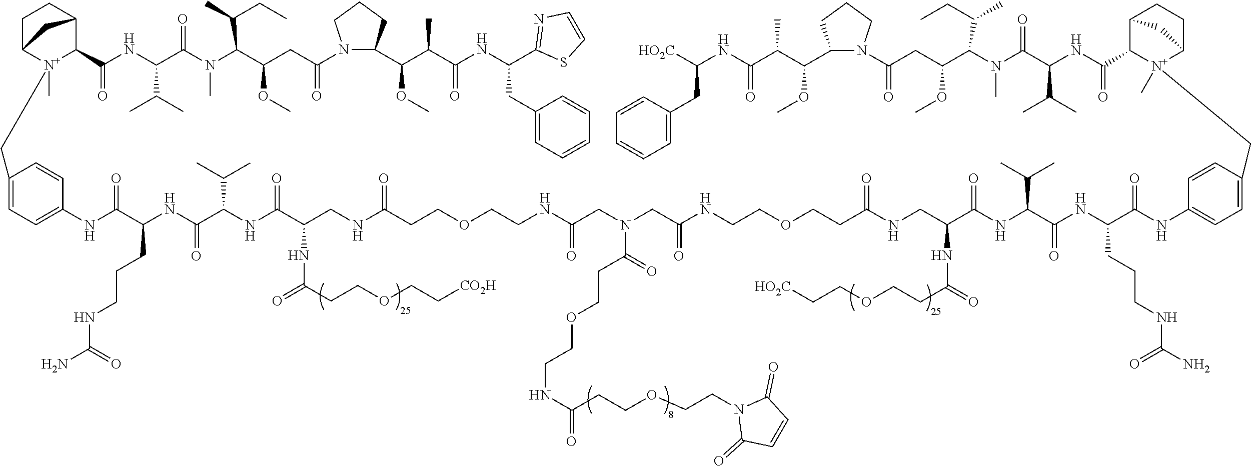 |
| P3-L3- P3 | 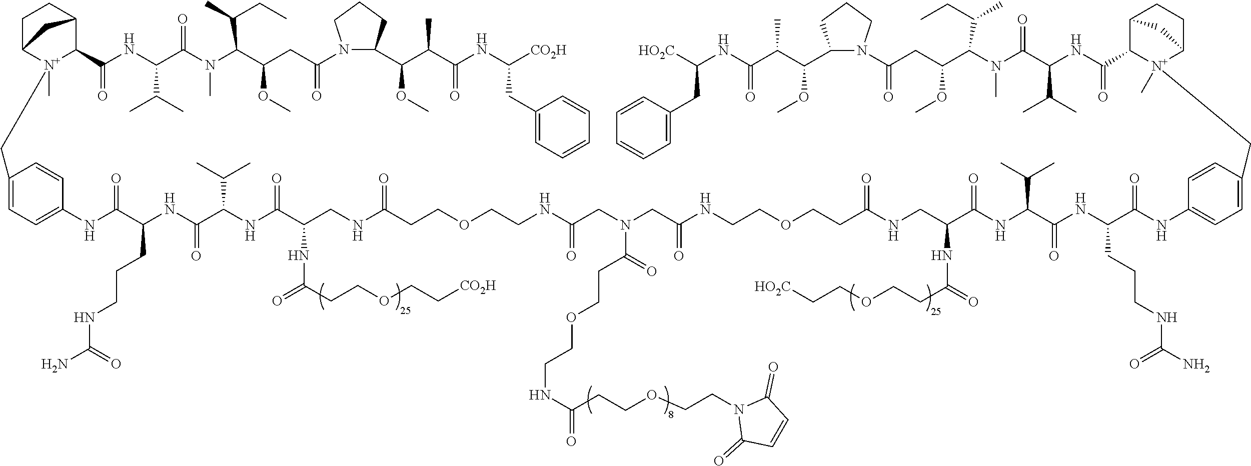 |
| P3-L3- P4 | 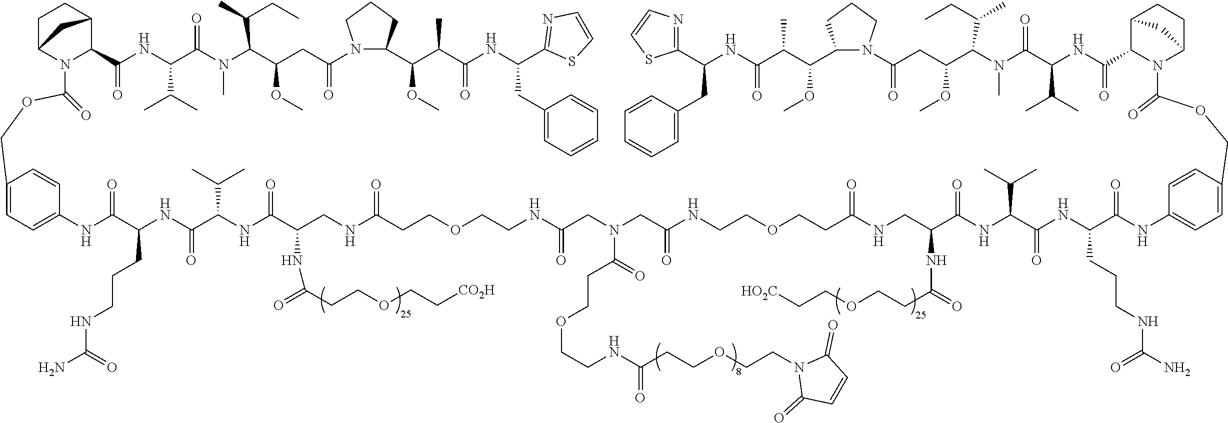 |
| P4-L3- P4 | 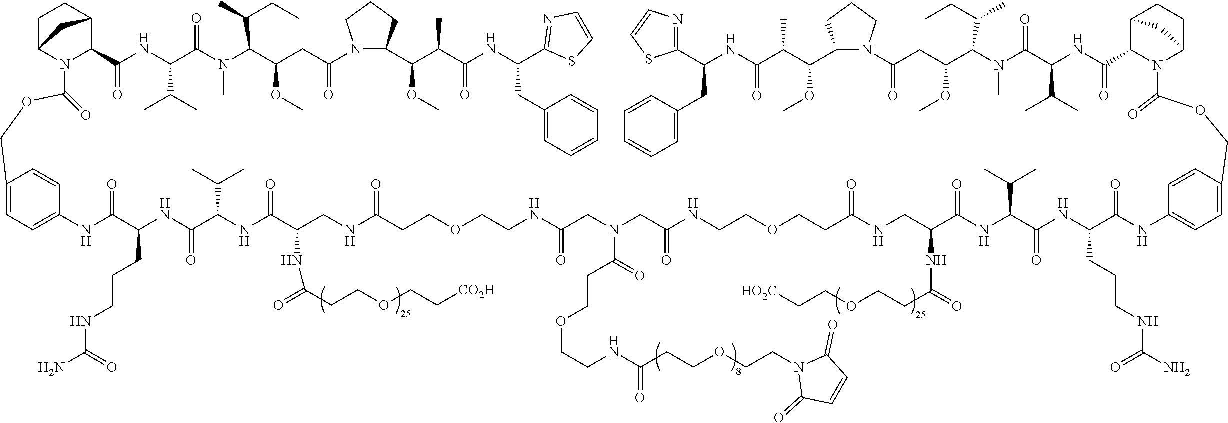 915 |
| P1-L4- P1 | 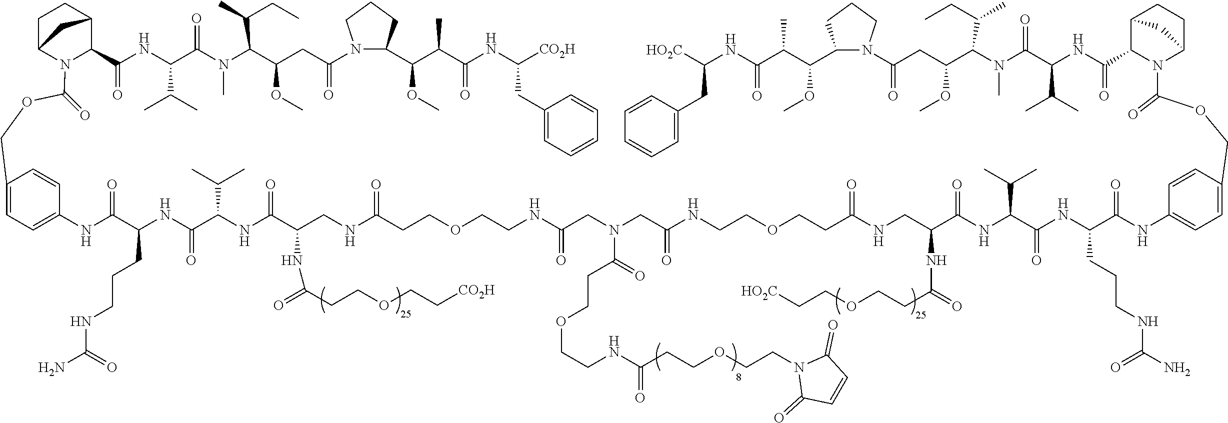 |
| P1-L4- P2 | 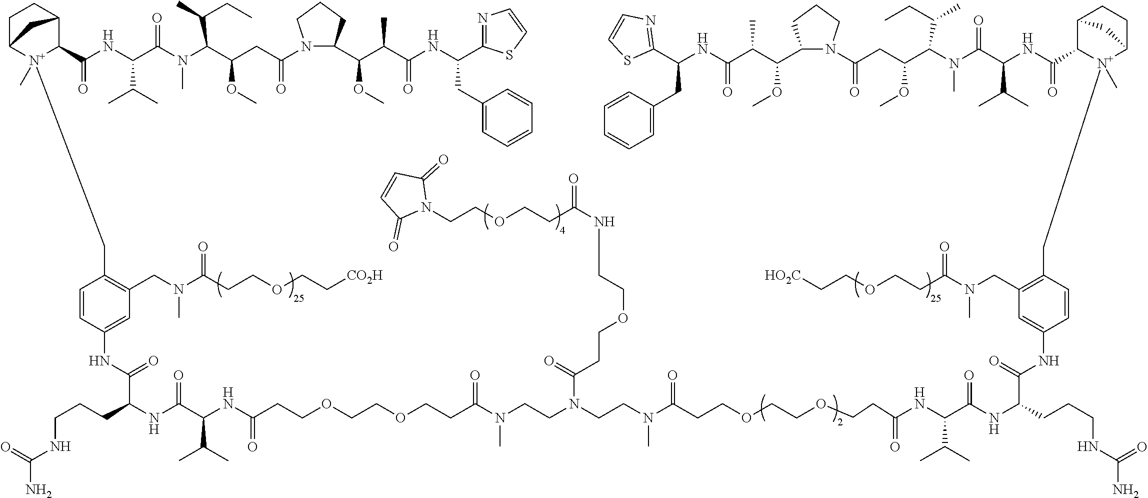 |
| P2-L4- P1 | 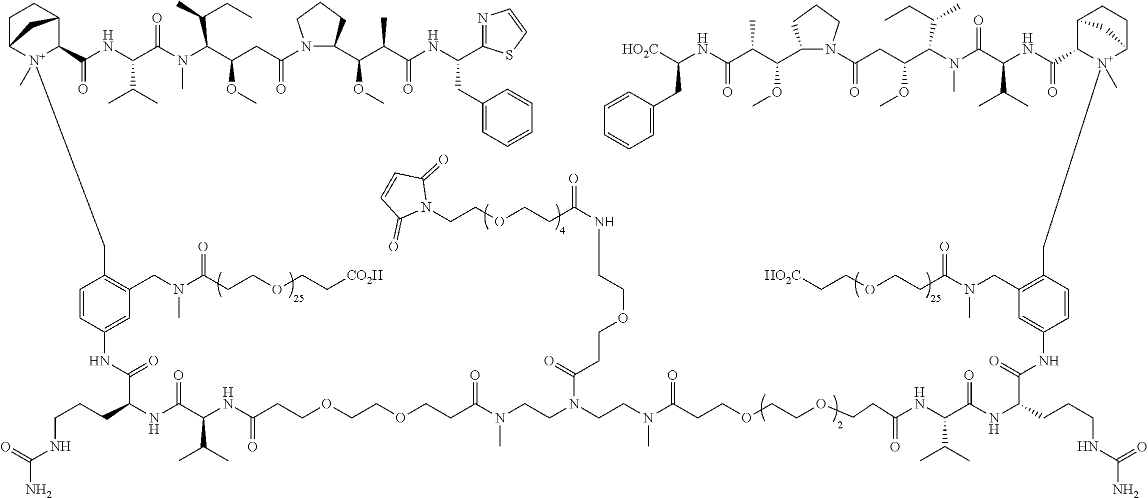 |
| P2-L4- P2 | 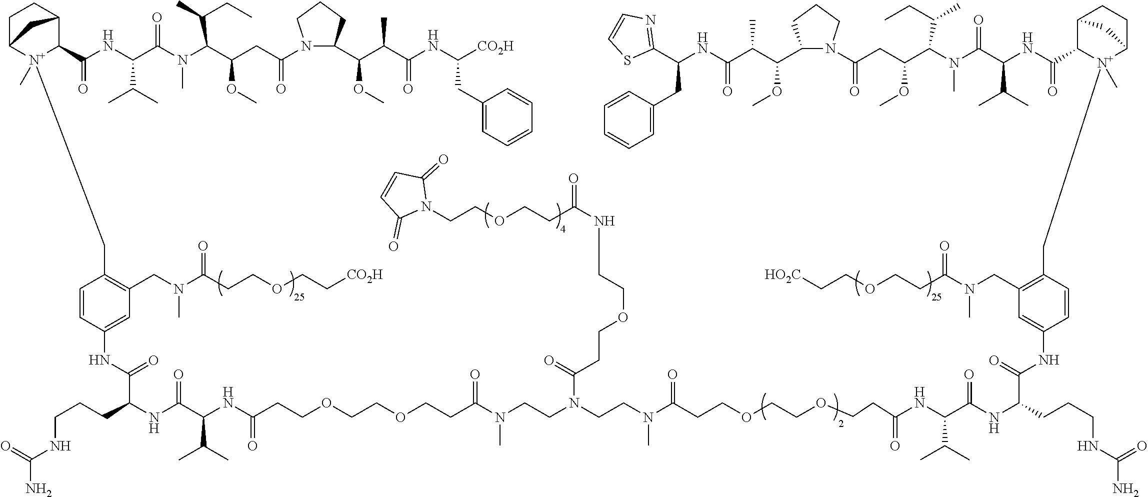 |
| P3-L4- P3 | 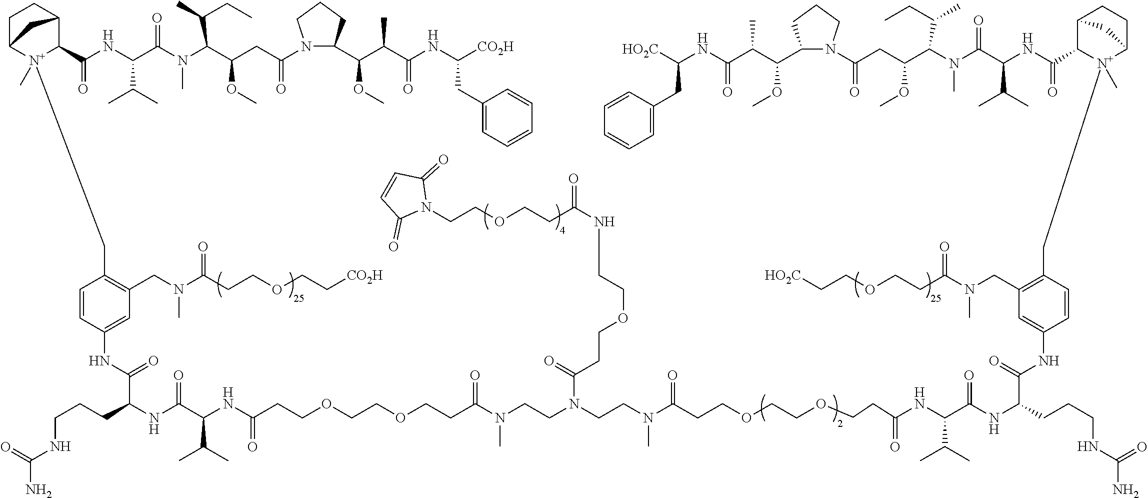 |
| P3-L4- P4 | 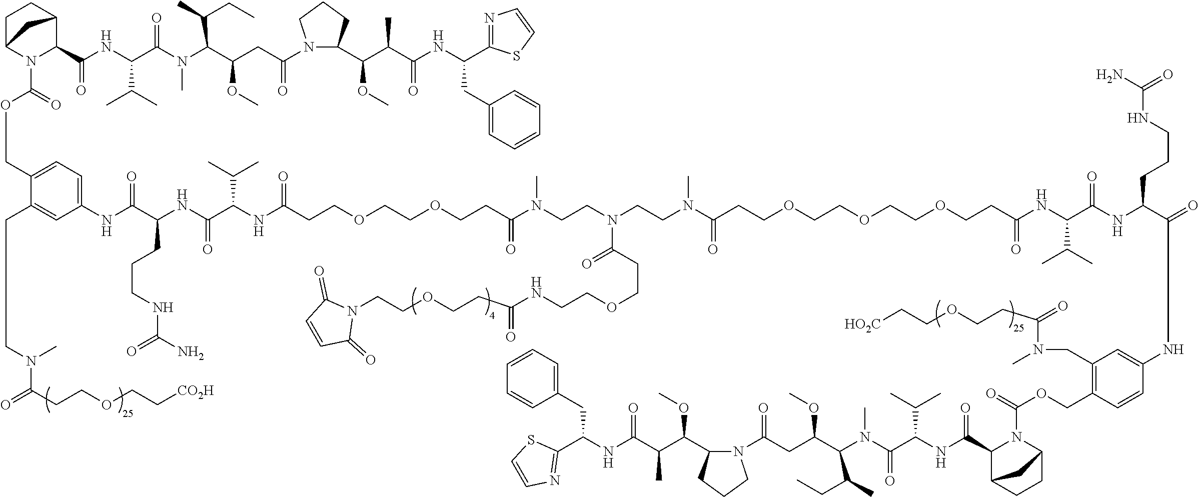 |
| P4-L4- P3 | 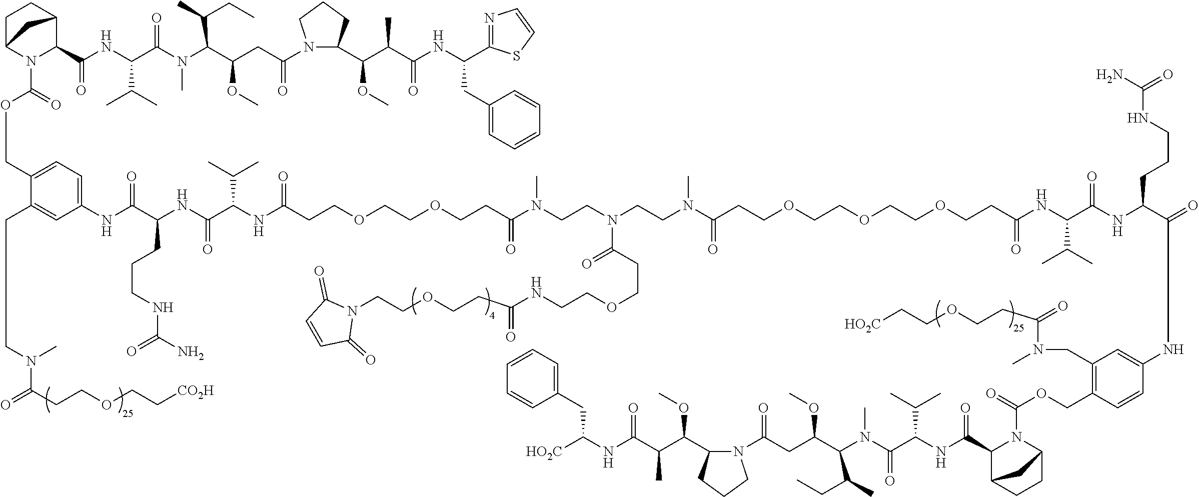 |
| P4-L4- P4 | 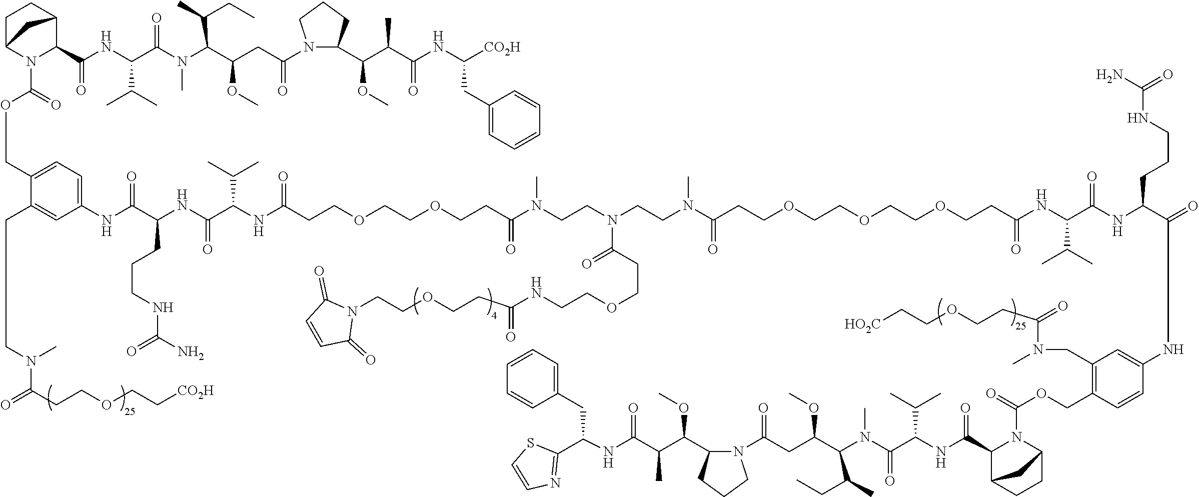 |
| P5-L4- P5 | 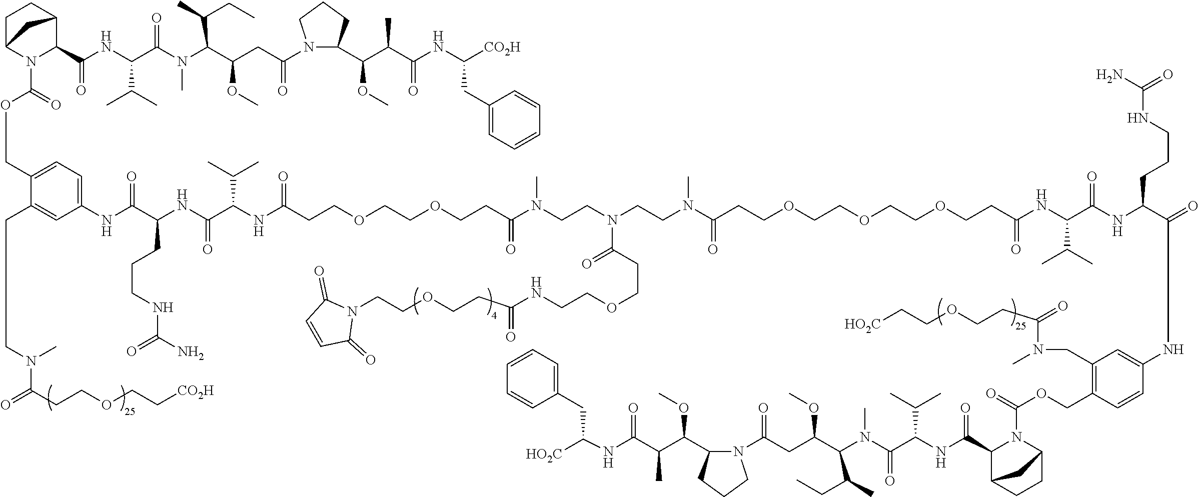 |
| P6-L4- P6 | 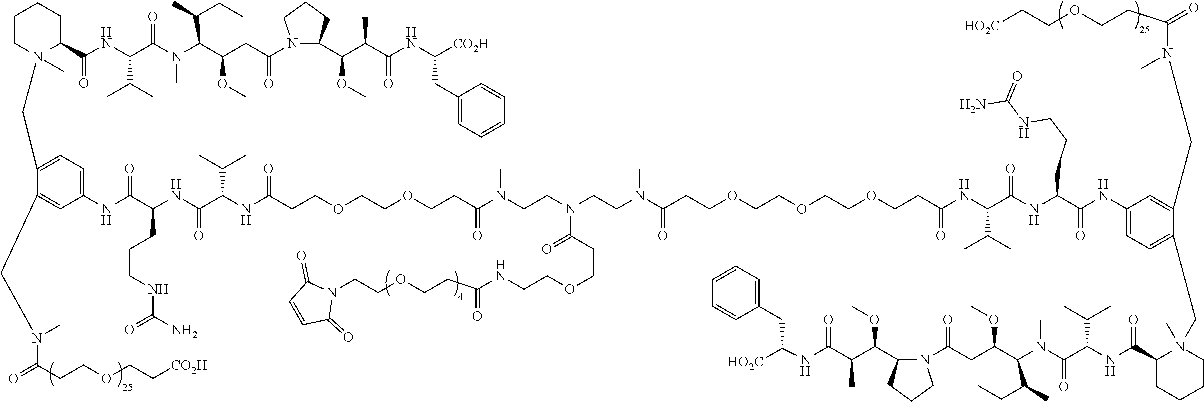 |
| P1-L5- P1 | 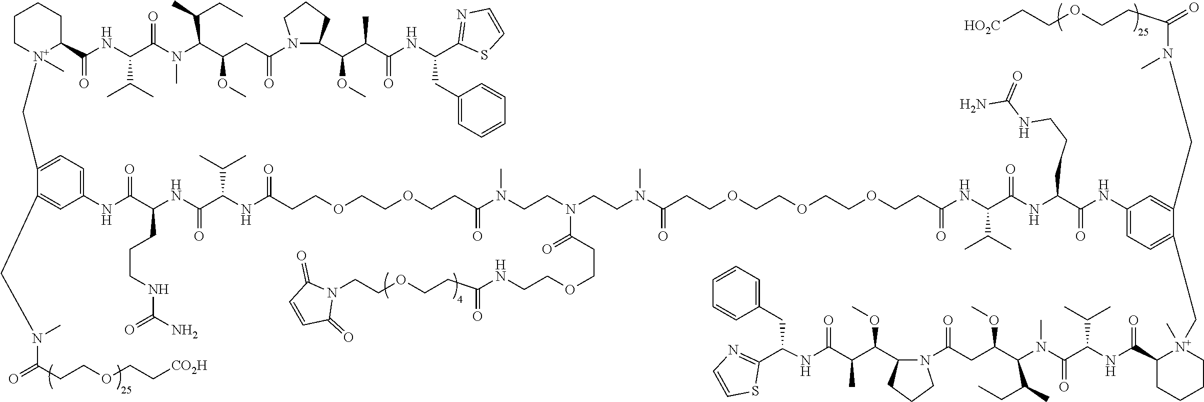 |
| P1-L5- P2 | 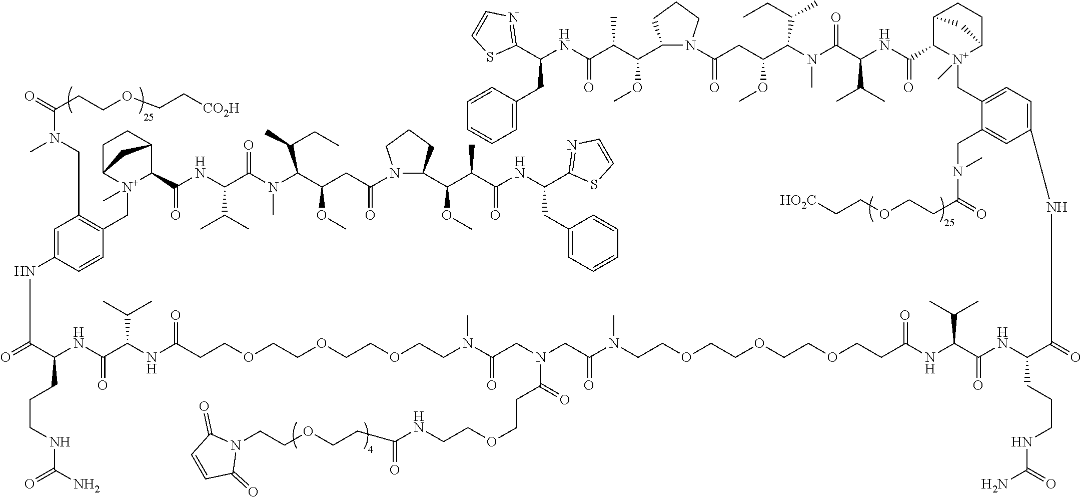 |
| P2-L5- P2 | 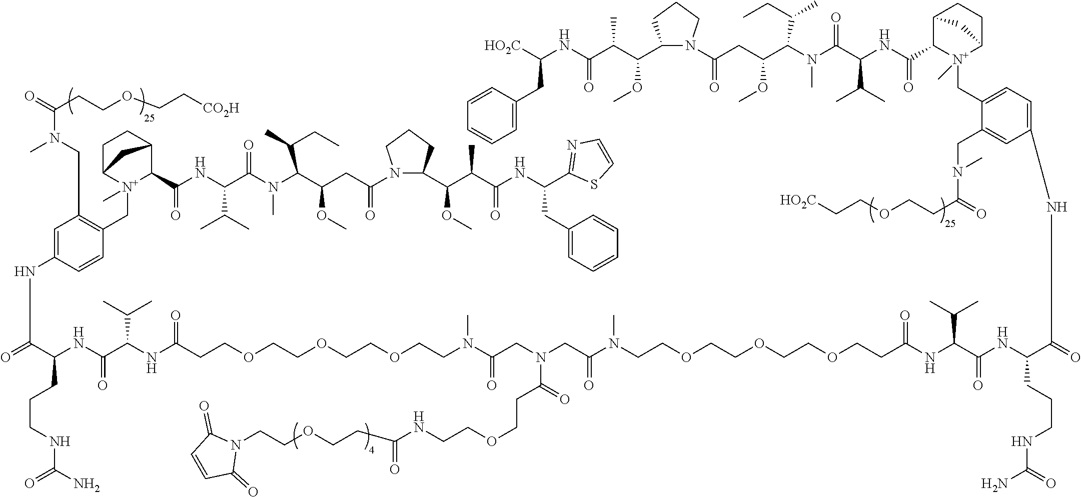 |
| P3-L5- P3 | 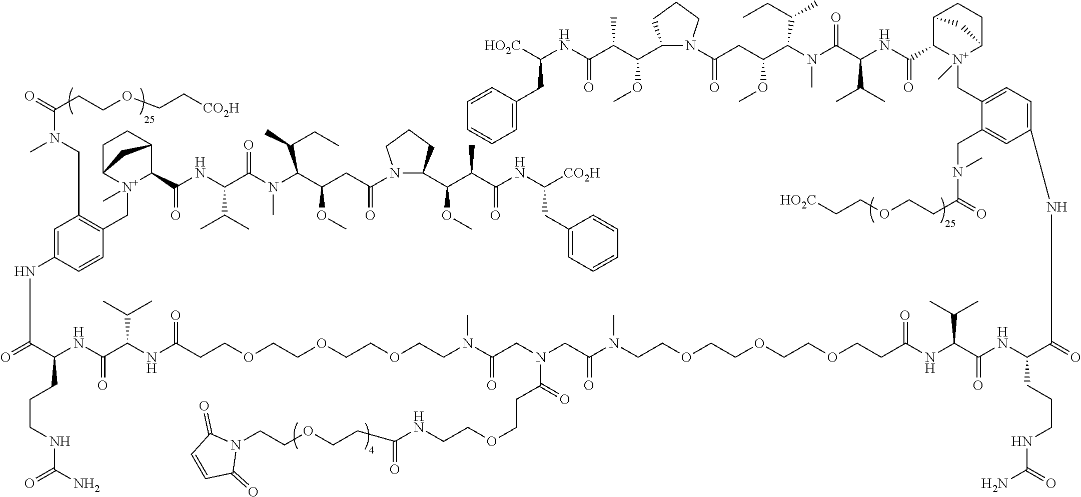 |
| P3-L5- P4 | 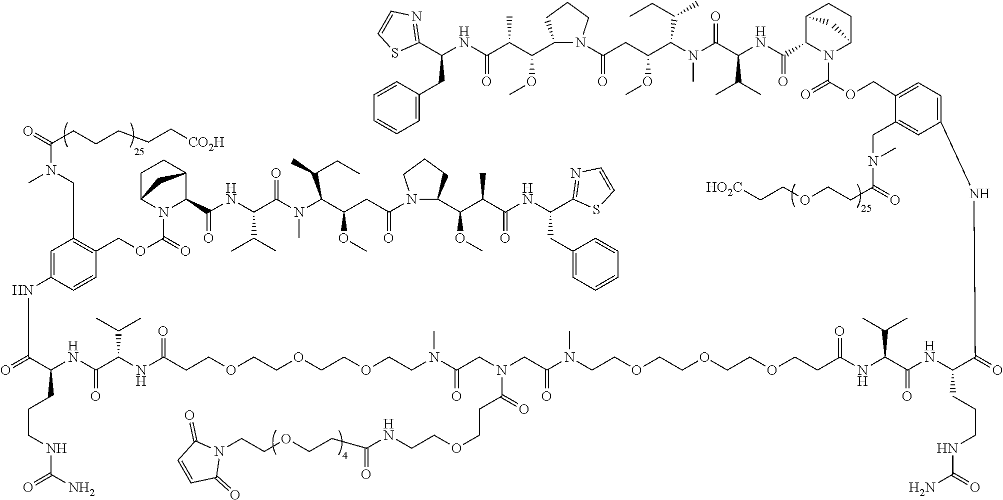 |
| P4-L5- P4 | 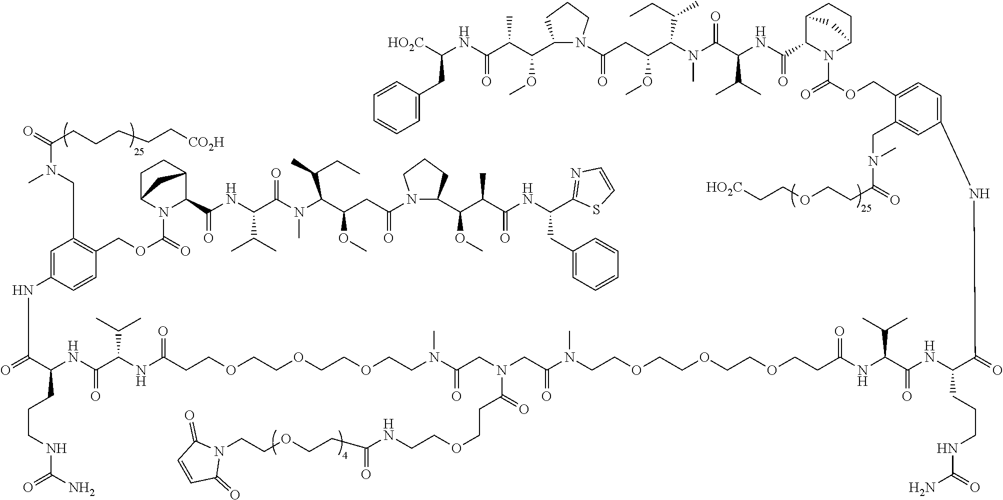 |
| P2-L5- P5 | 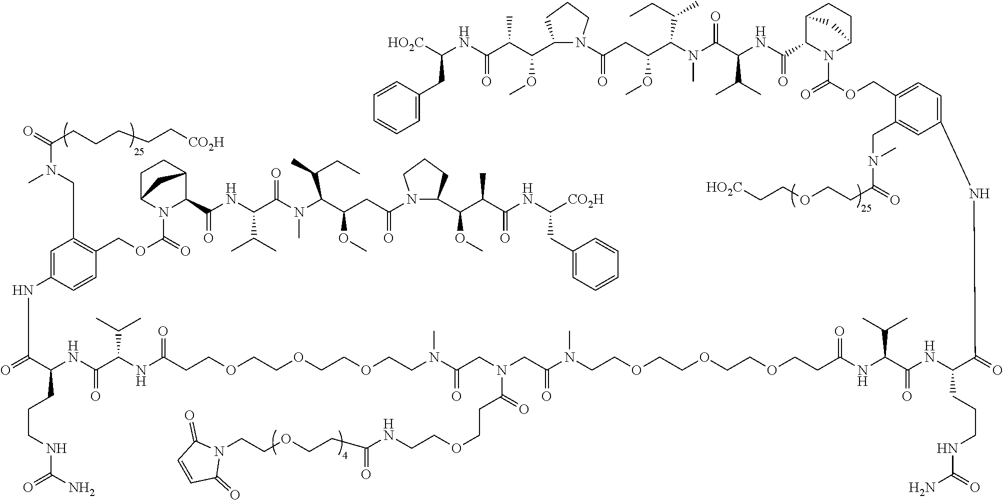 |
| P5-L5- P5 | 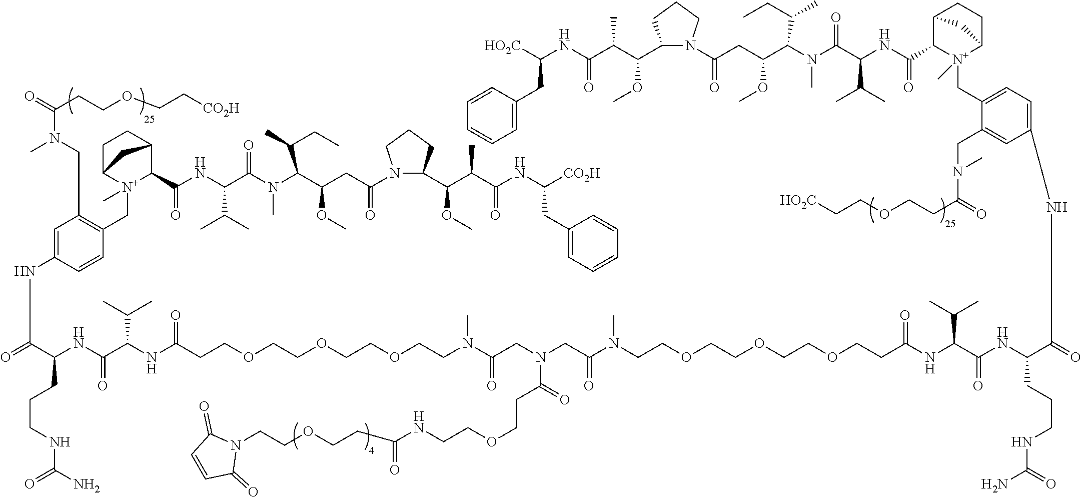 |
| P5-L5- P7 | 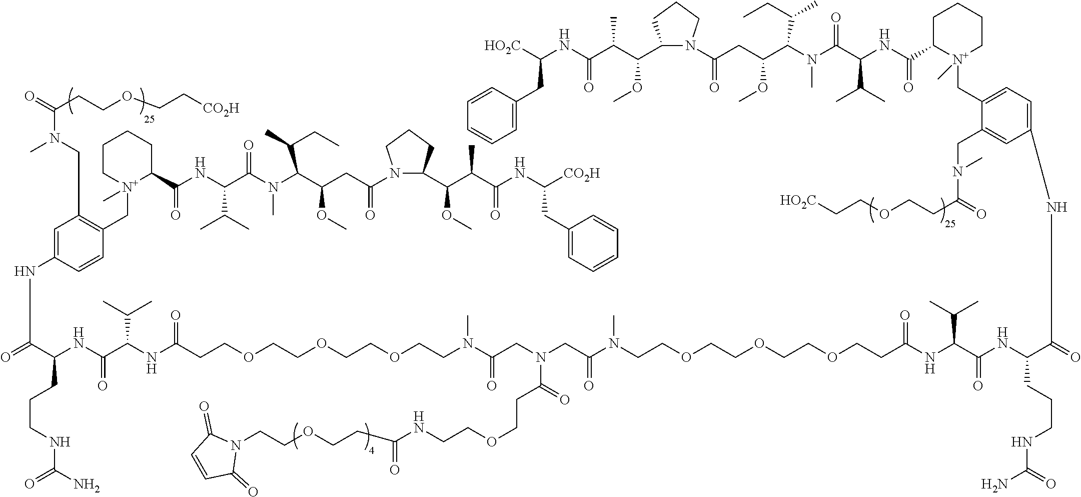 |
| P5-L5- P8 | 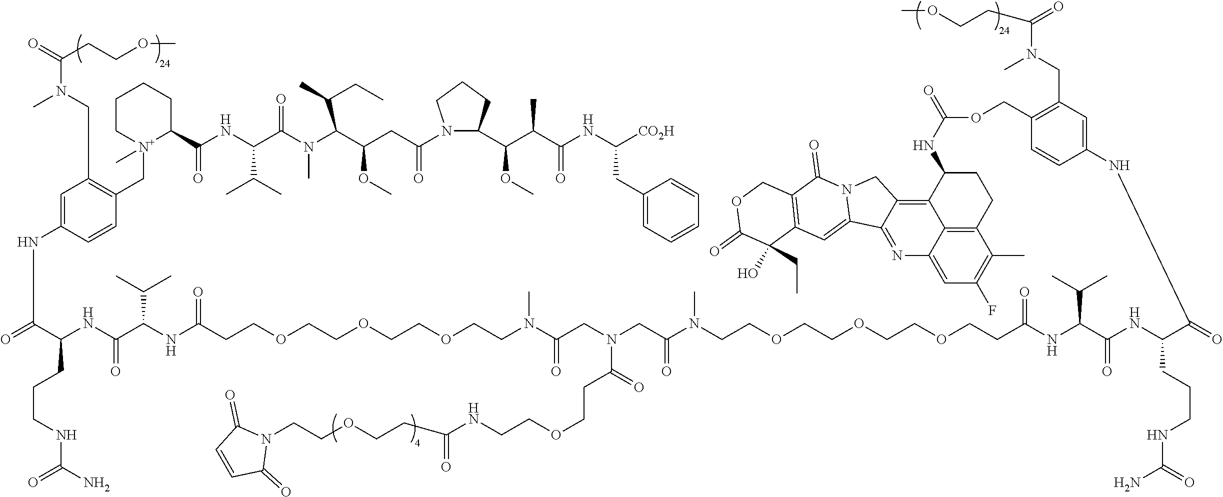 |
| P6-L5- P6 | 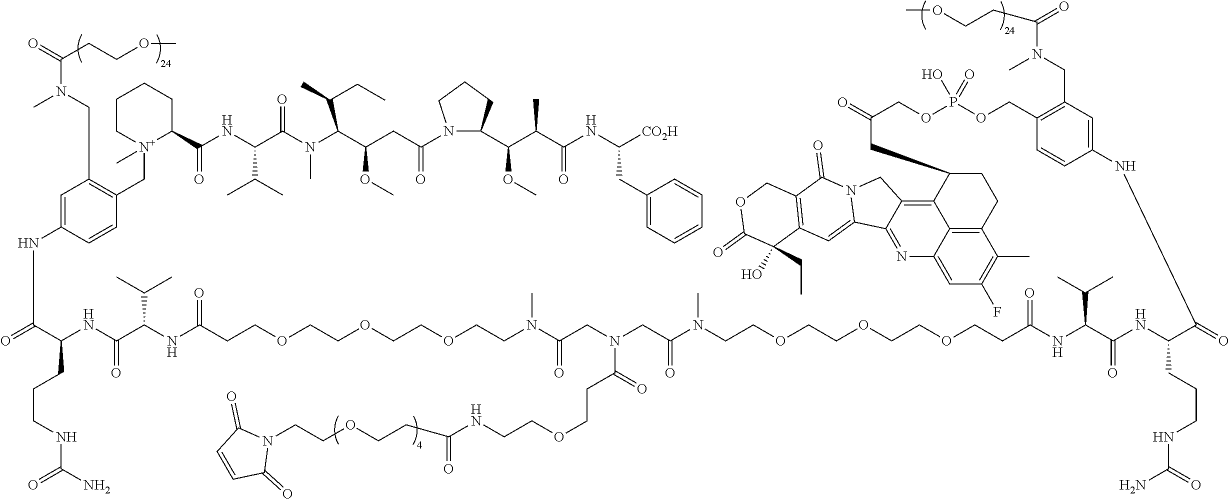 |
| P7-L5- P7 | 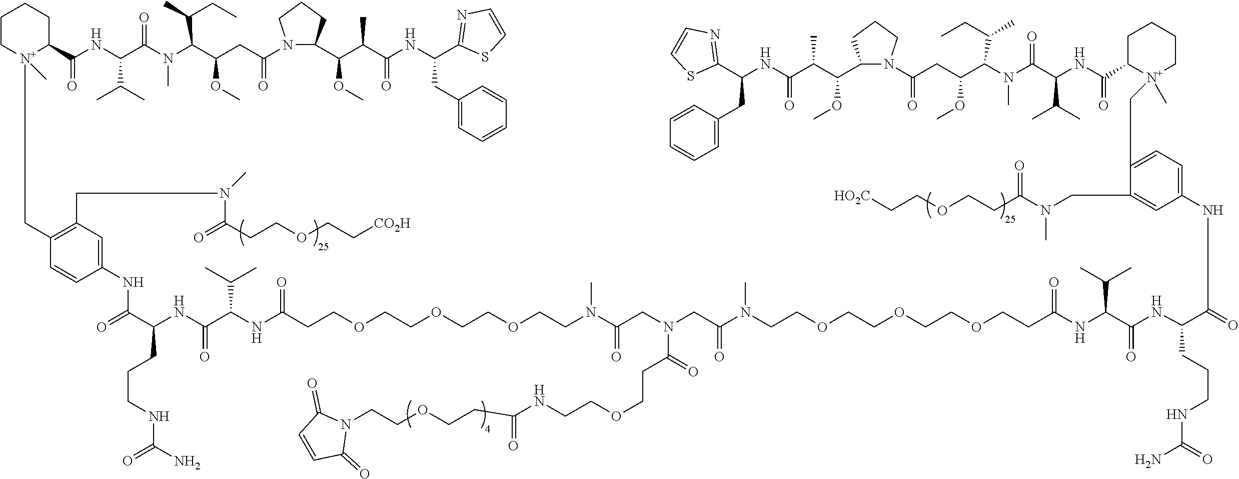 |
| P8-L5- P8 | 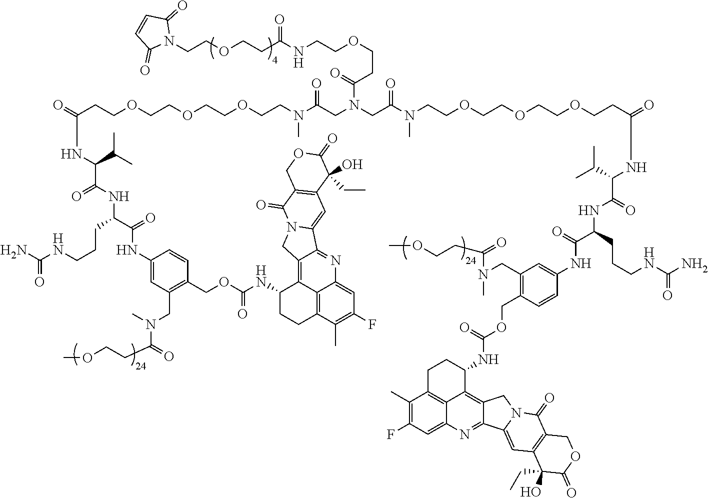 |
| P1-L6- P1 | 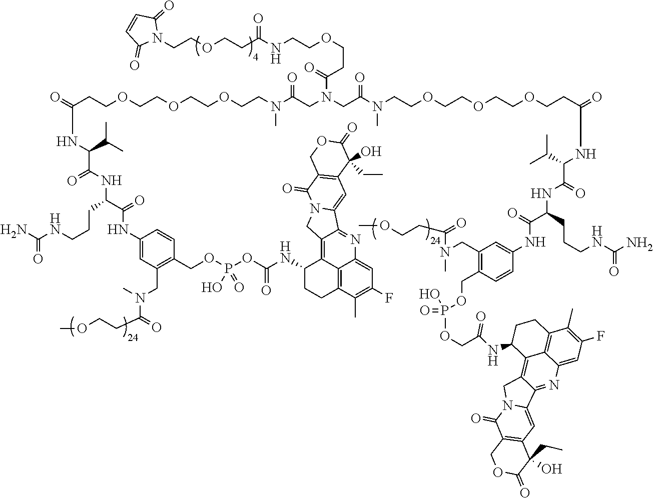 |
| P1-L6- P2 | 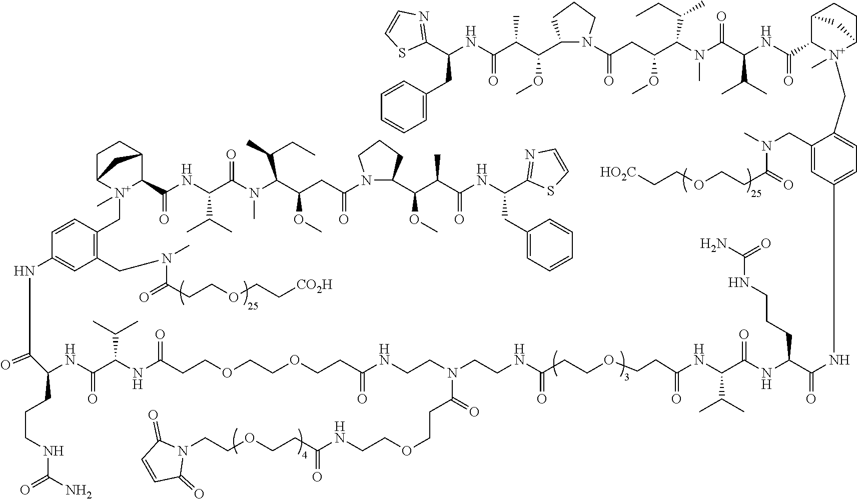 |
| P1-L6- P3 | 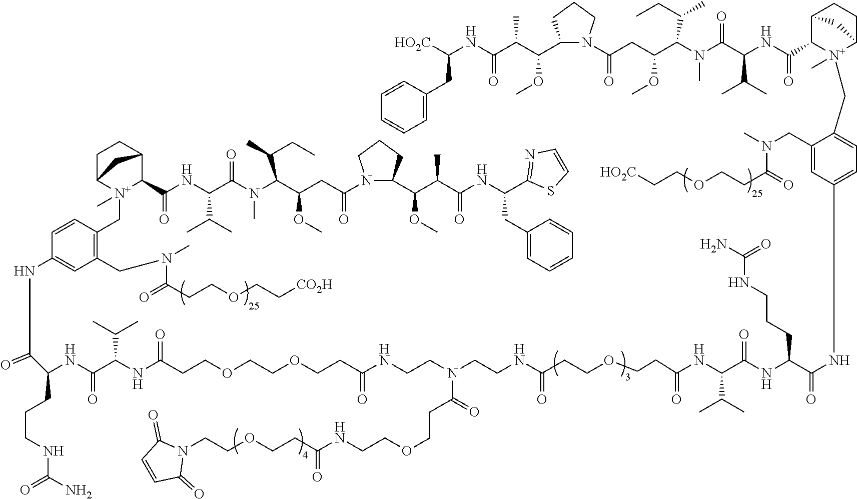 |
| P1-L6- P4 | 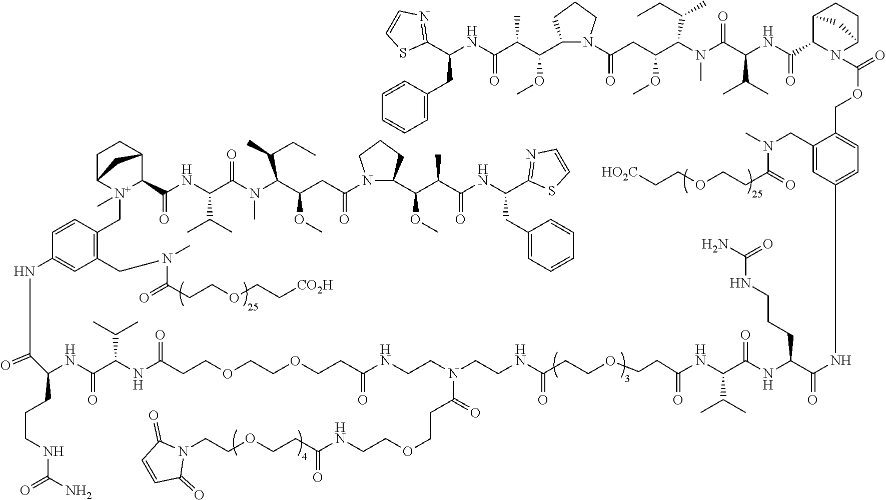 |
| P2-L6- P1 | 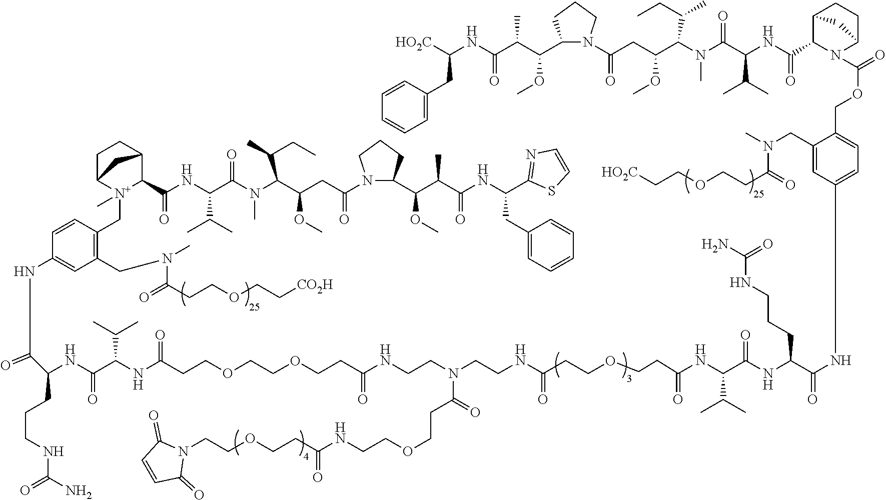 |
| P2-L6- P2 | 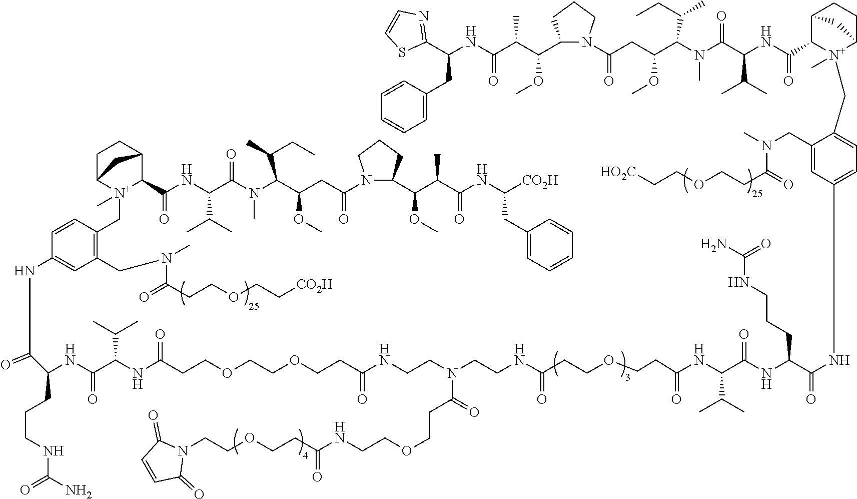 |
| P2-L6- P3 | 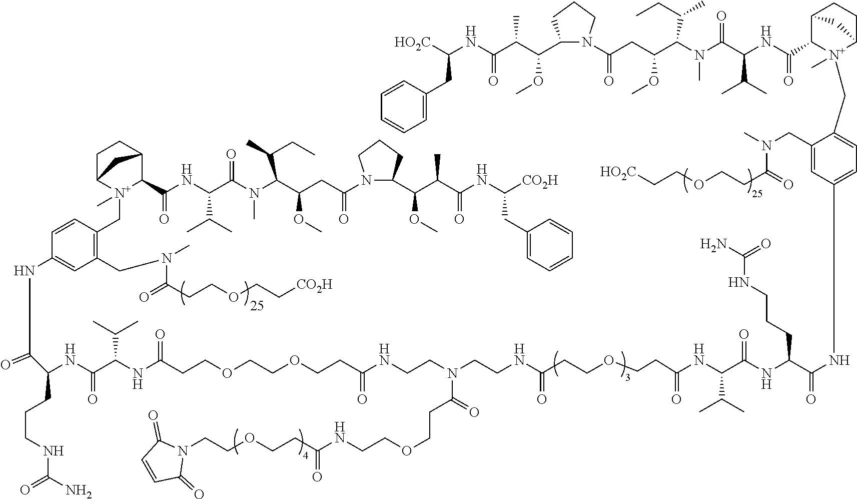 |
| P2-L6- P4 | 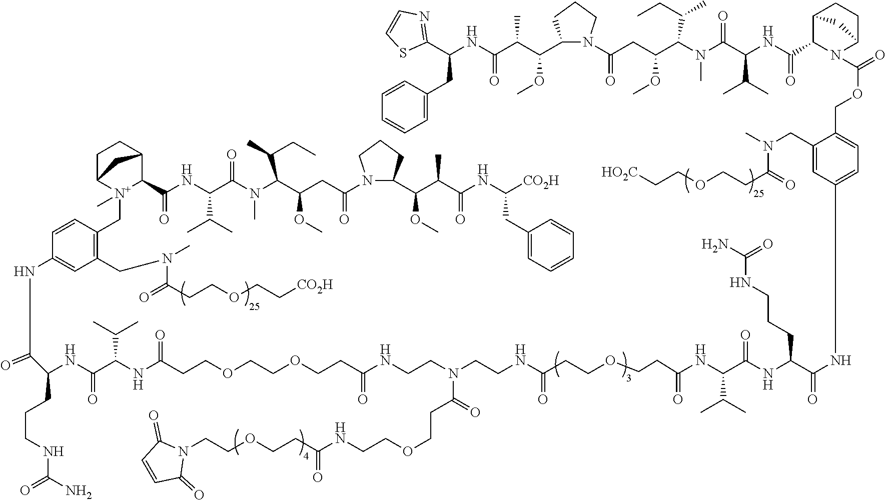 |
| P3-L6- P1 | 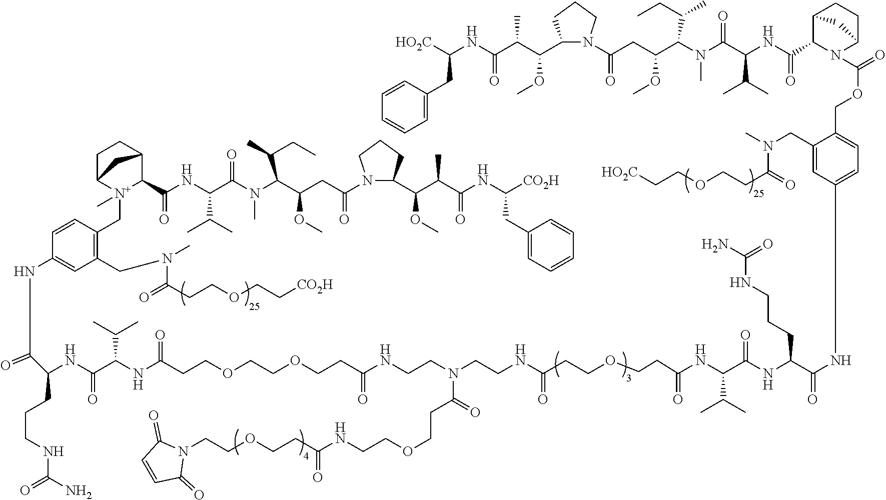 |
| P3-L6- P2 | 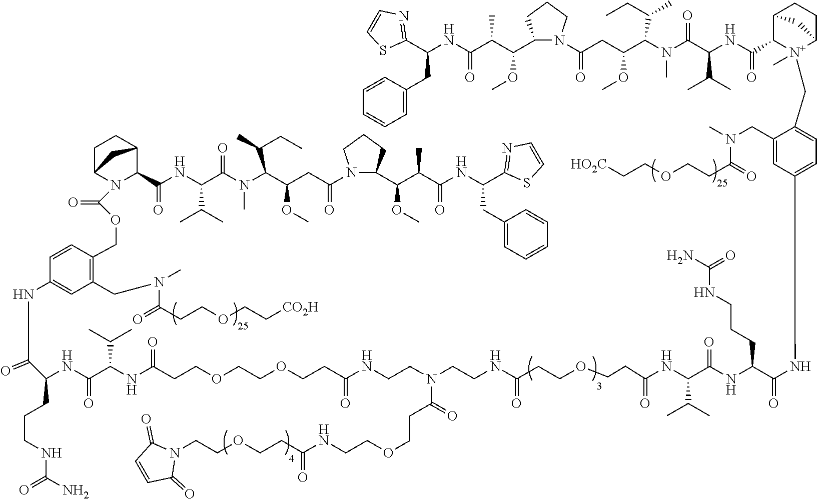 |
| P3-L6- P3 | 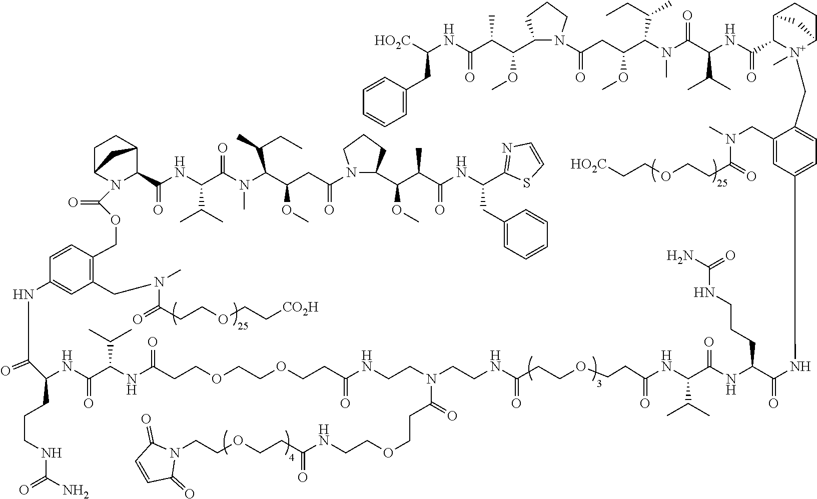 |
| P3-L6- P4 | 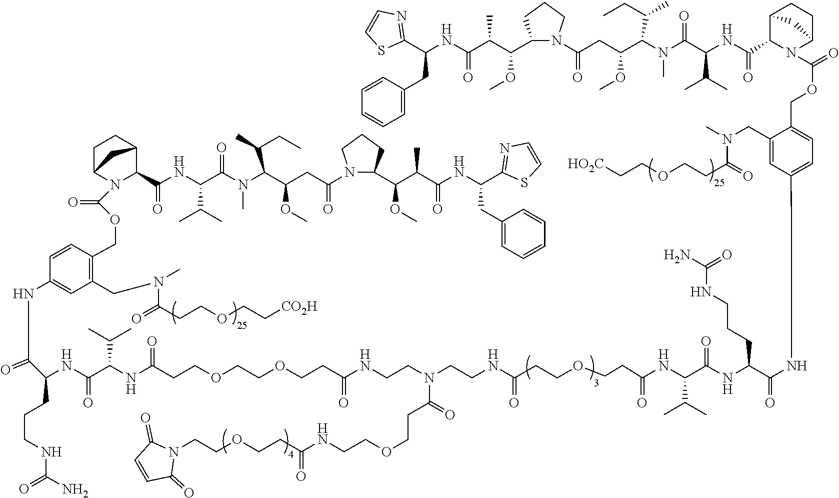 |
| P4-L6- P1 | 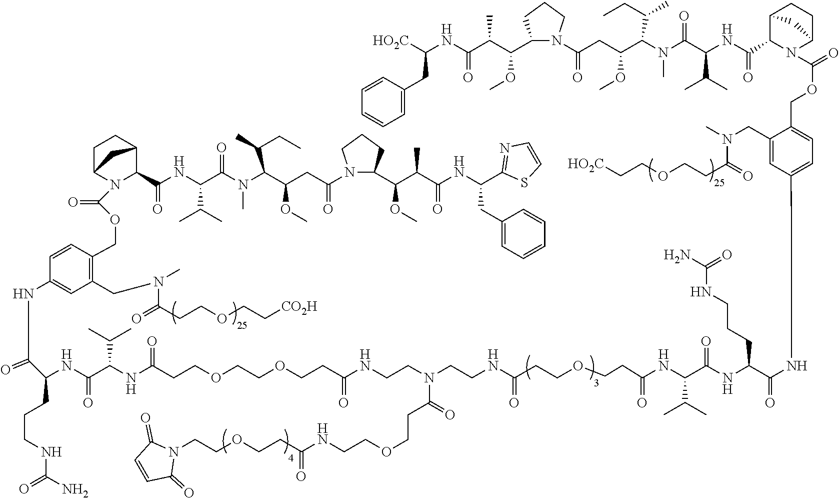 |
| P4-L6- P2 | 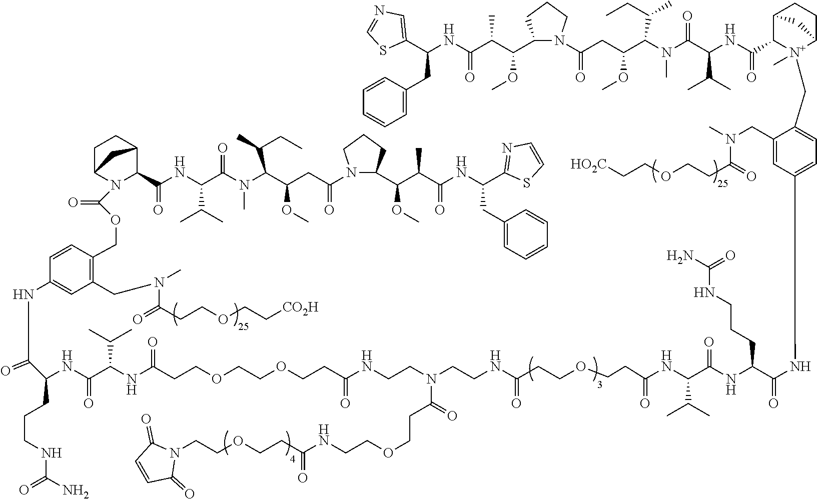 |
| P4-L6- P3 | 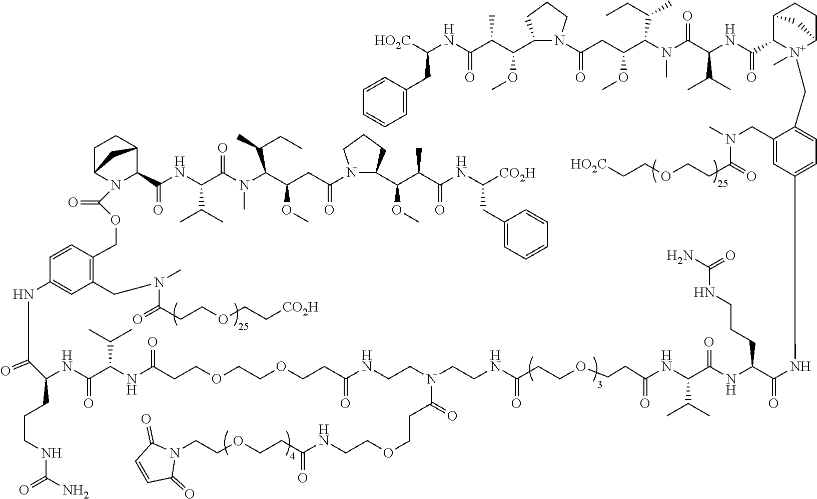 |
| P4-L6- P4 | 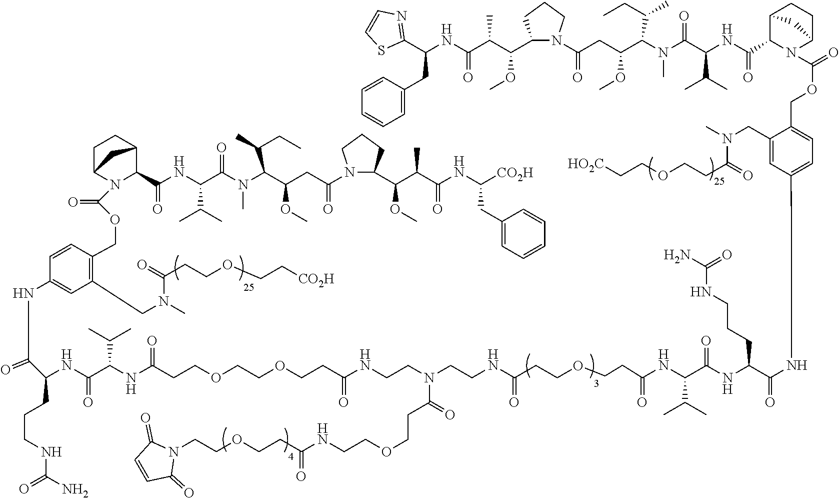 |
| P5-L6- P3 | 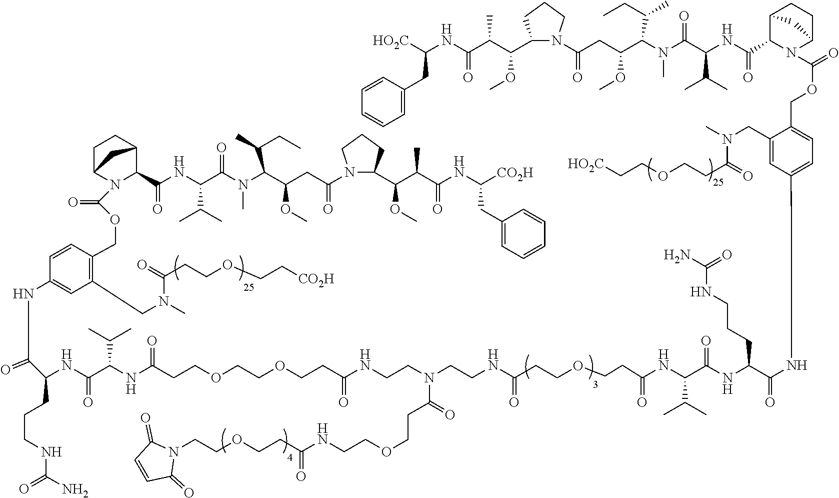 |
| P6-L6- P6 | 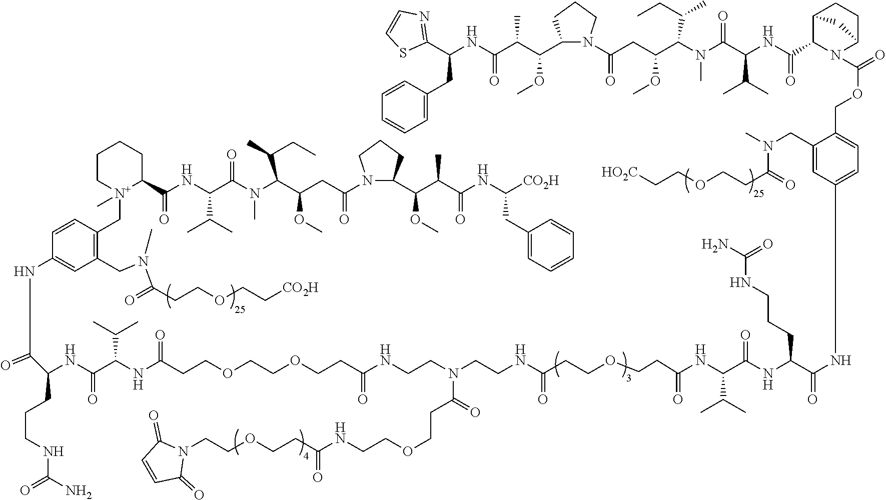 |
| P7-L6- P7 | 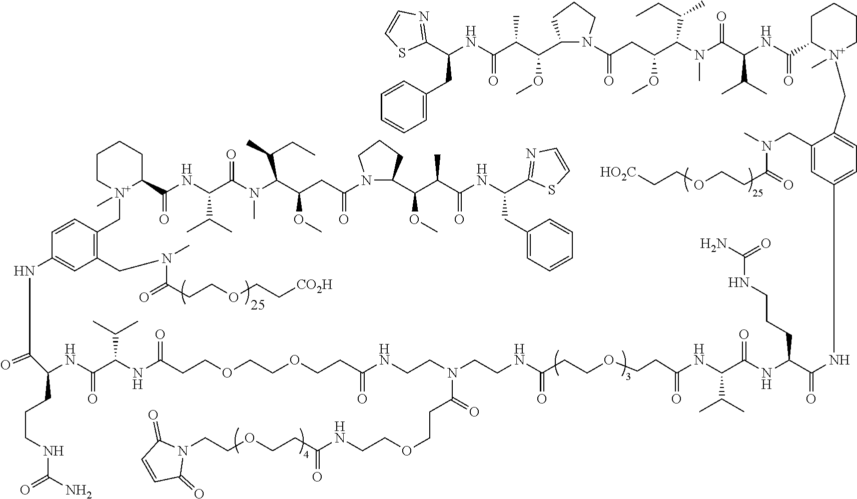 |
| P8-L6- P8 | 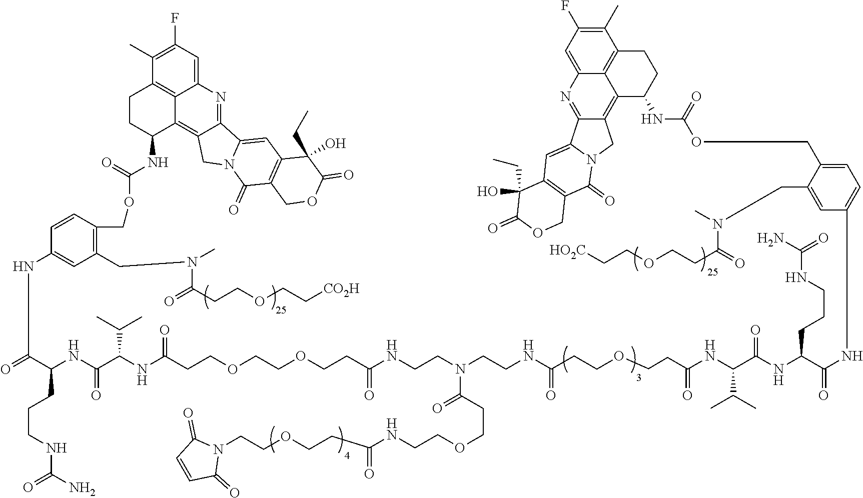 |
| P1-L7- P1 | 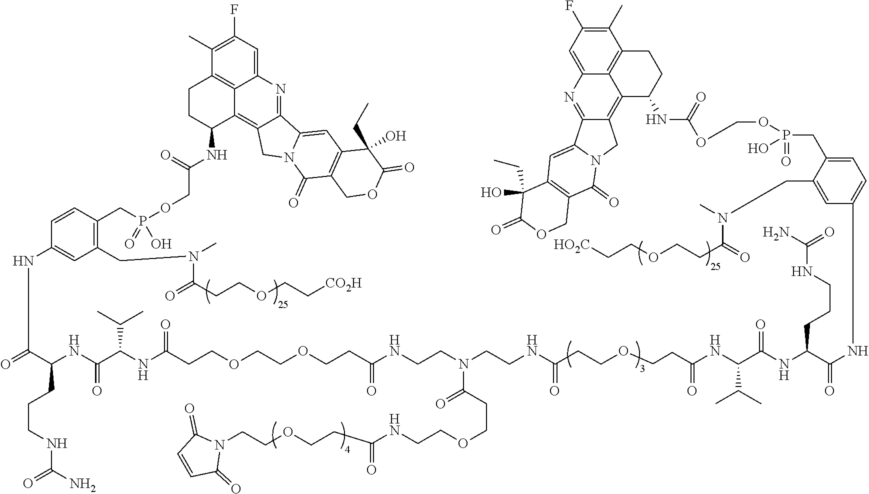 |
| P1-L7- P2 | 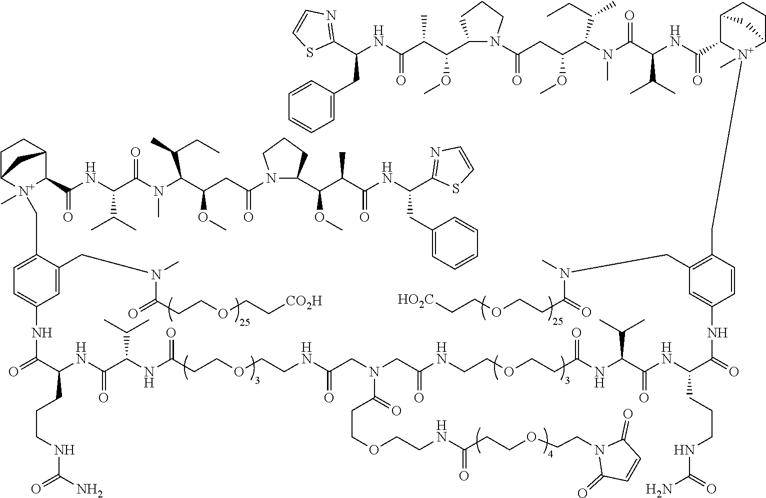 |
| P2-L7- P2 | 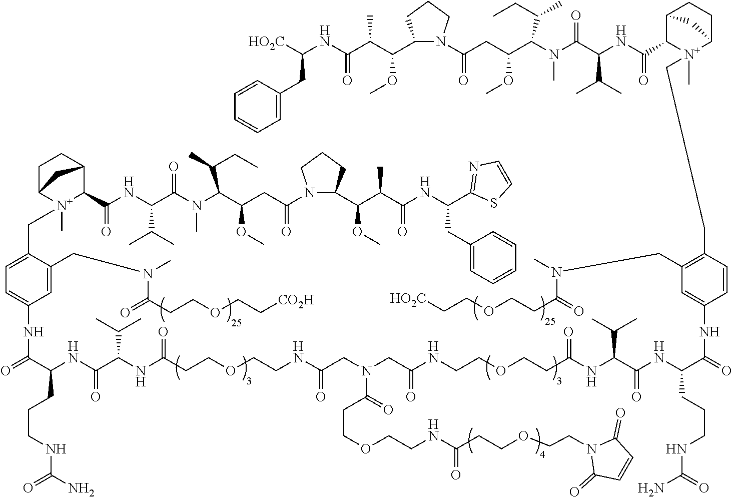 |
| P3-L7- P3 | 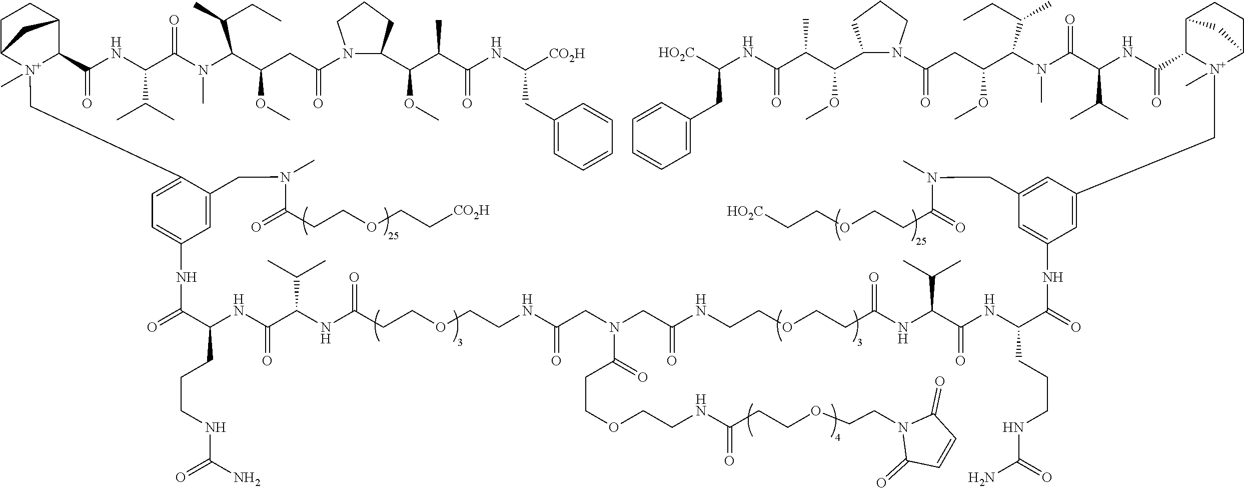 |
| P3-L7- P4 | 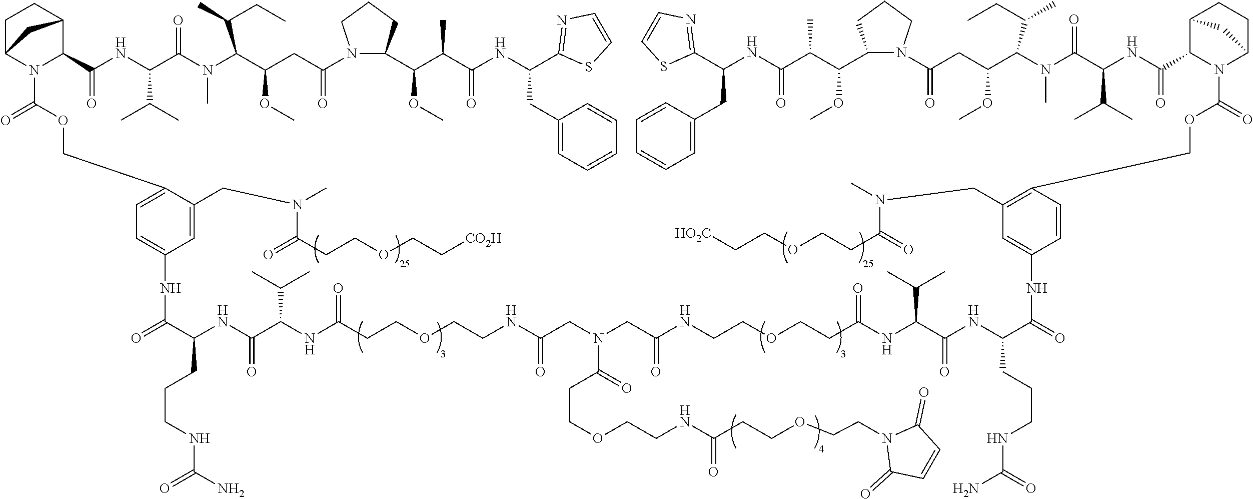 |
| P4-L7- P4 | 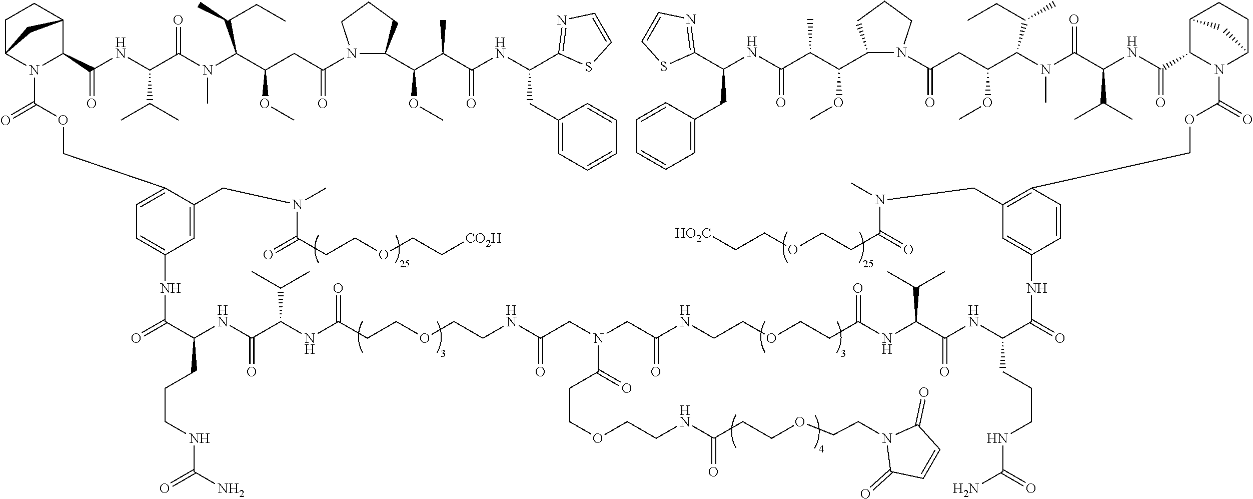 |
| P2-L7- P5 | 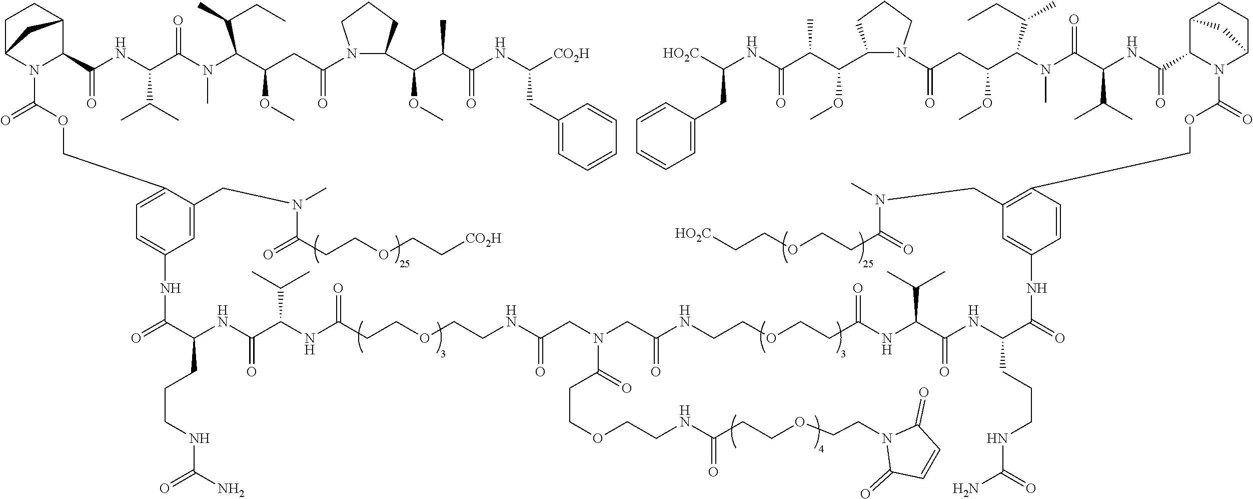 |
| P5-L7- P5 | 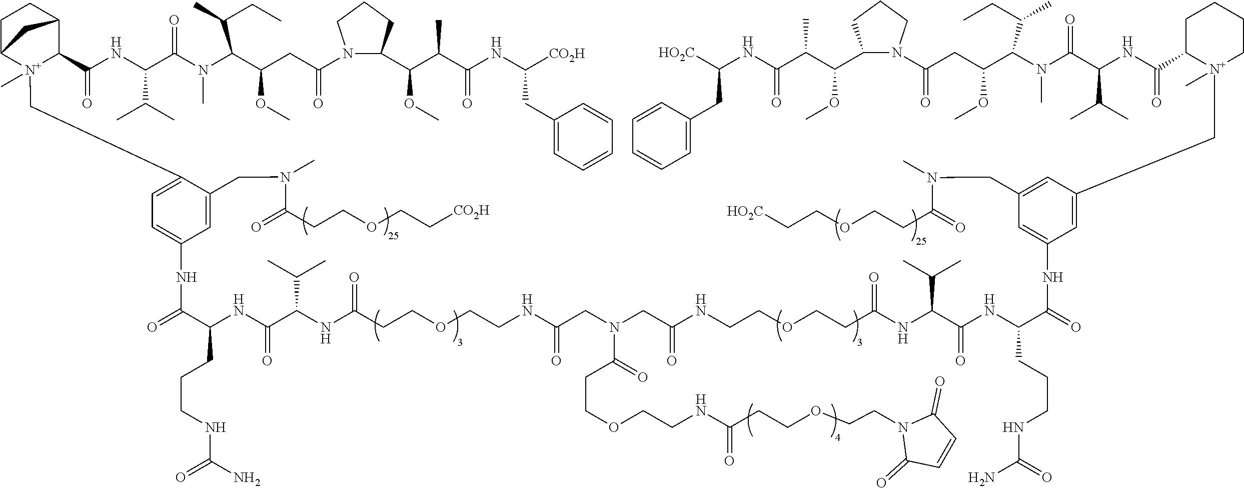 |
| P5-L7- P7 | 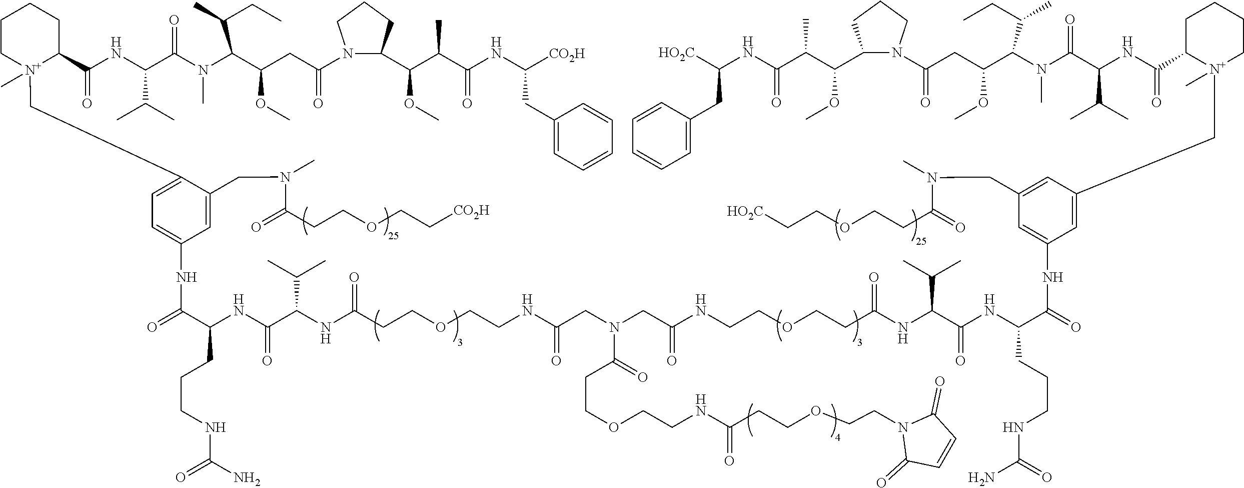 |
| P5-L7- P8 | 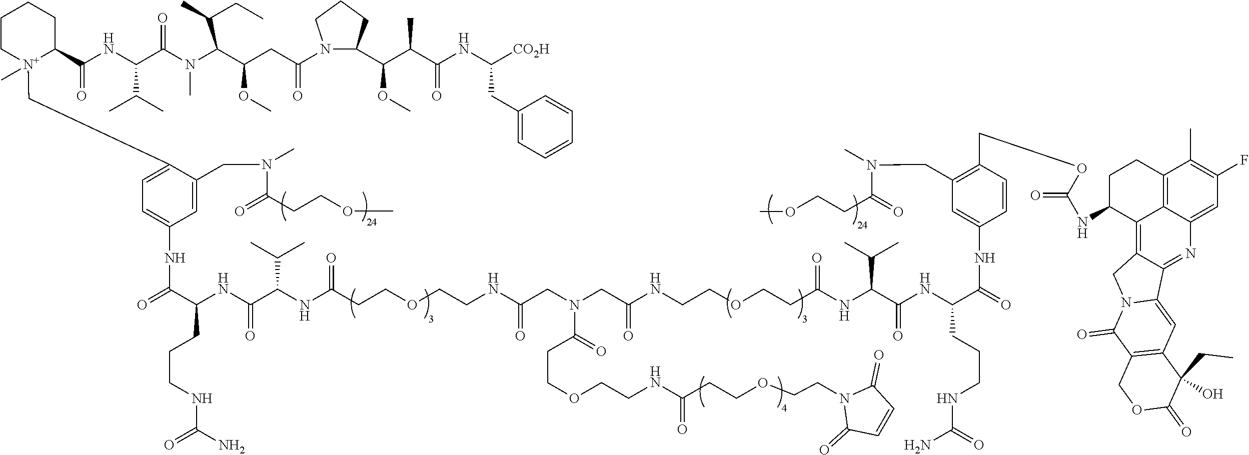 |
| P6-L7- P6 | 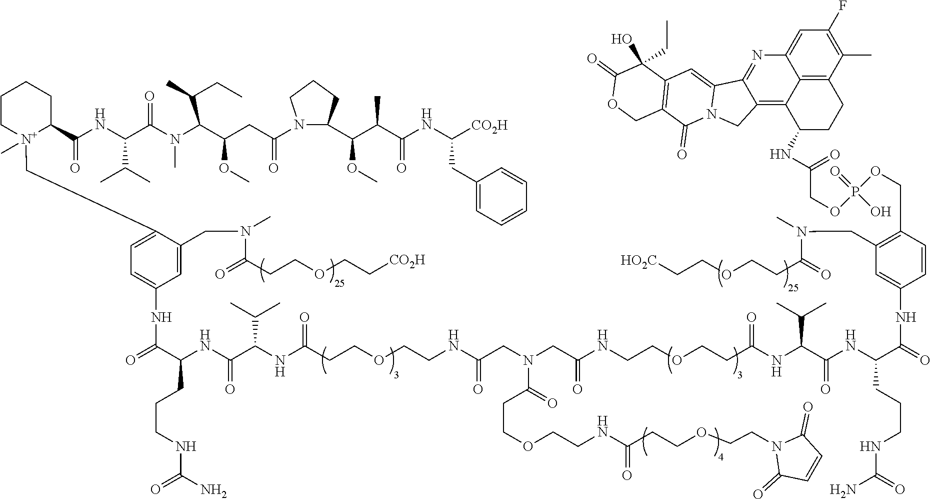 |
| P7-L7- P7 | 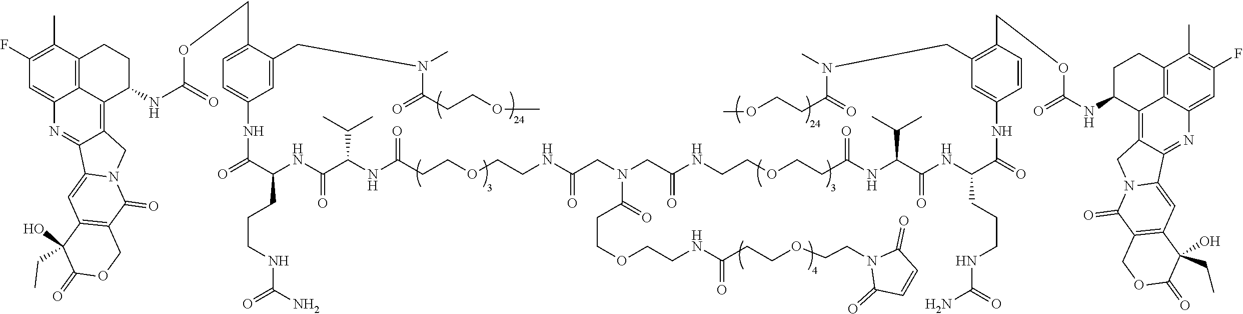 |
| P8-L7- P8 | 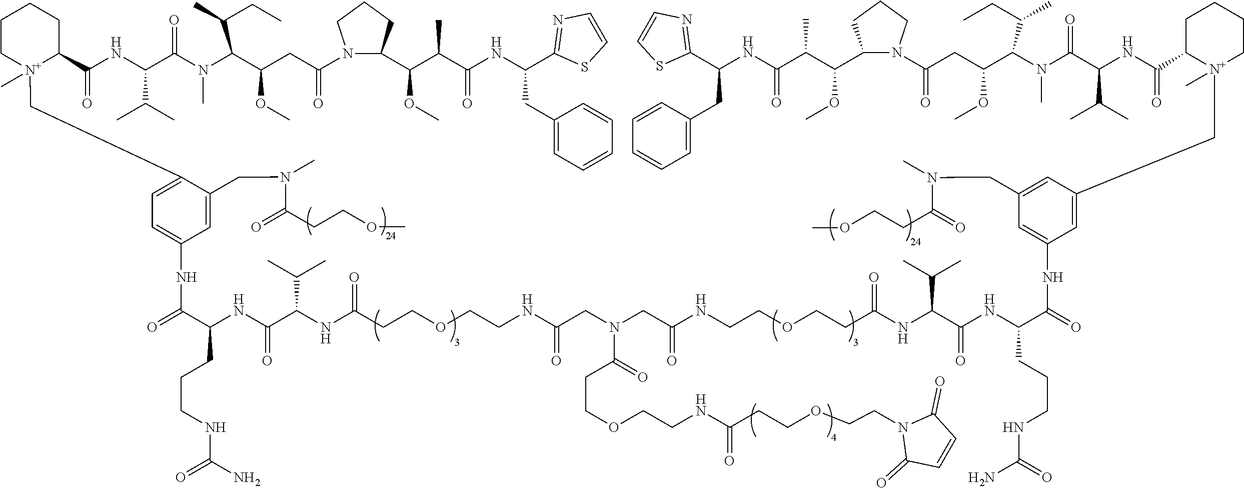 |
| P1-L8- P1 | 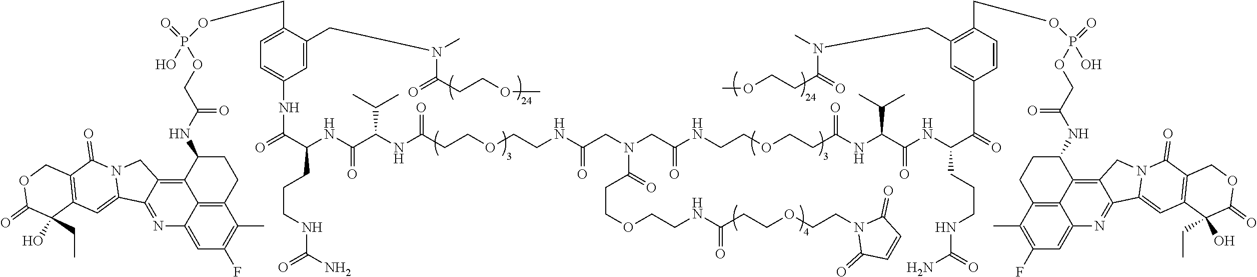 |
| P1-L8- P2 | 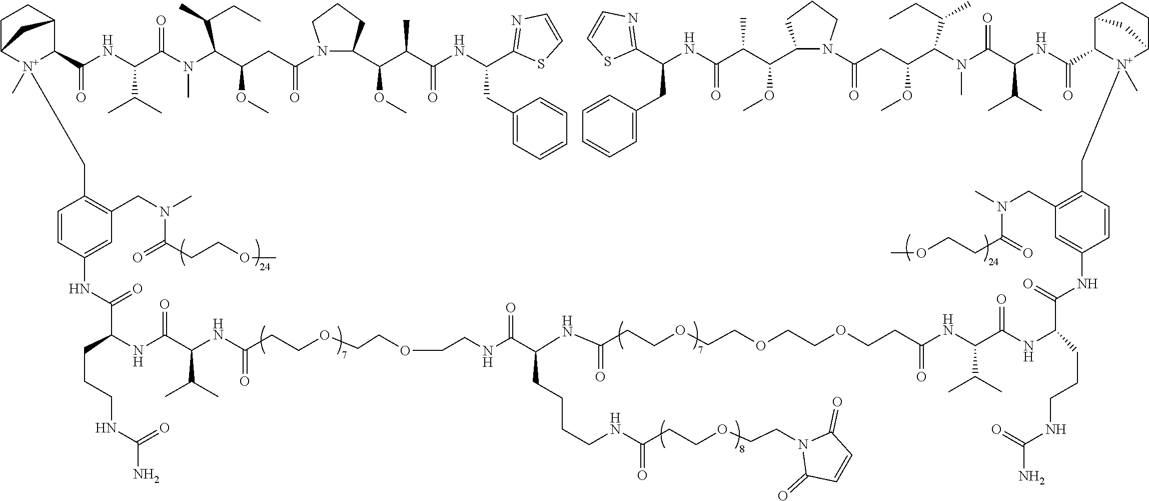 |
| P2-L8- P1 | 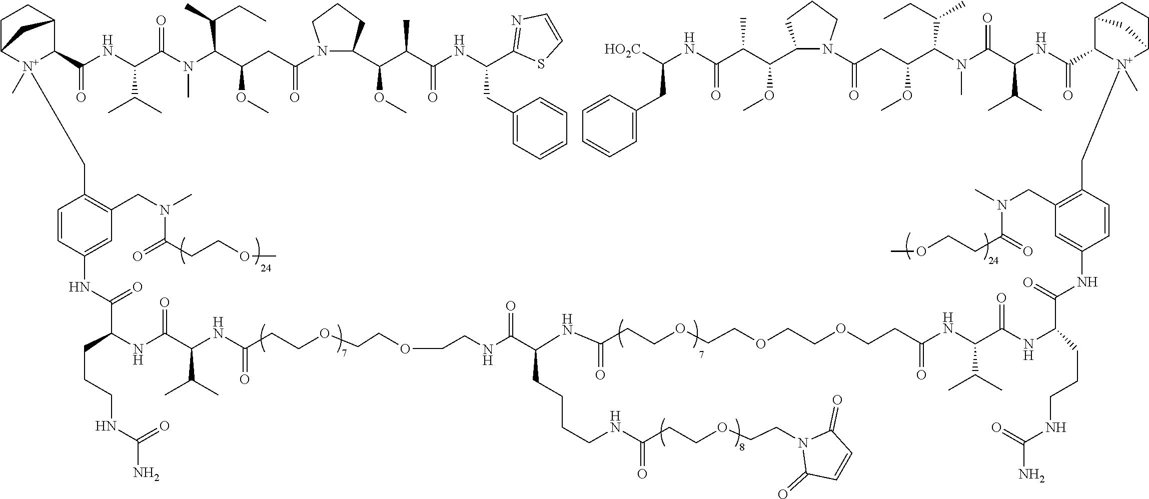 |
| P2-L8- P2 | 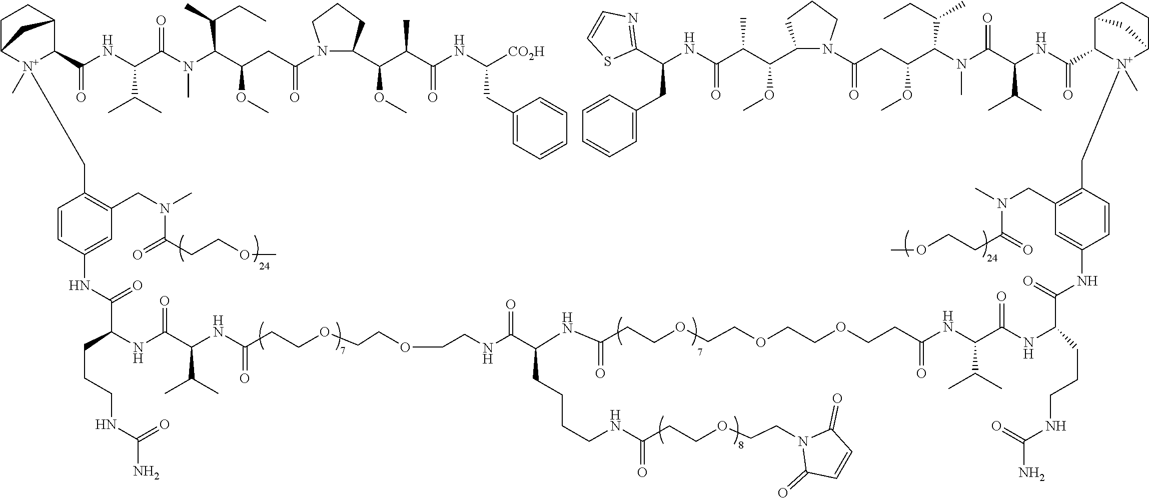 |
| P3-L8- P3 | 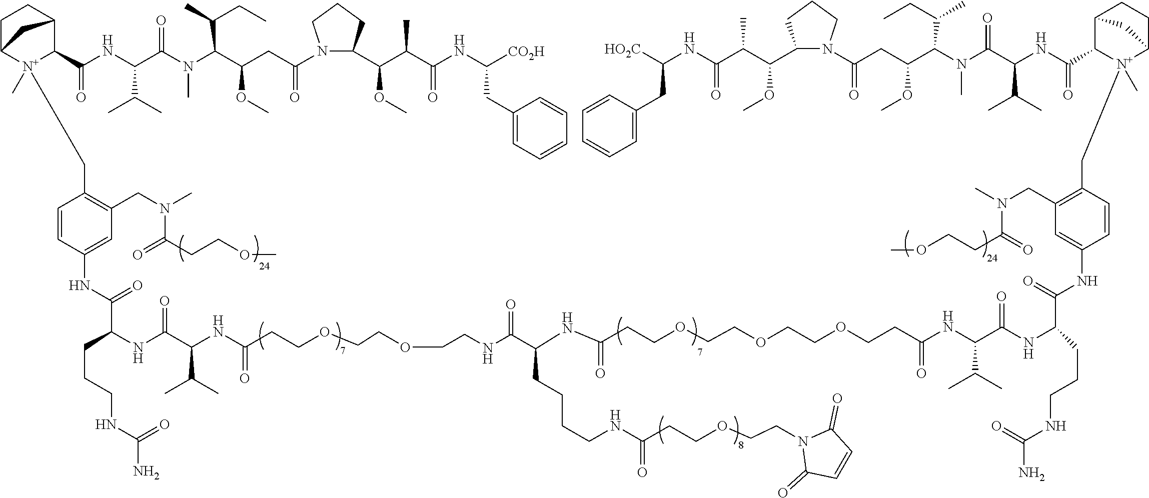 |
| P3-L8- P4 | 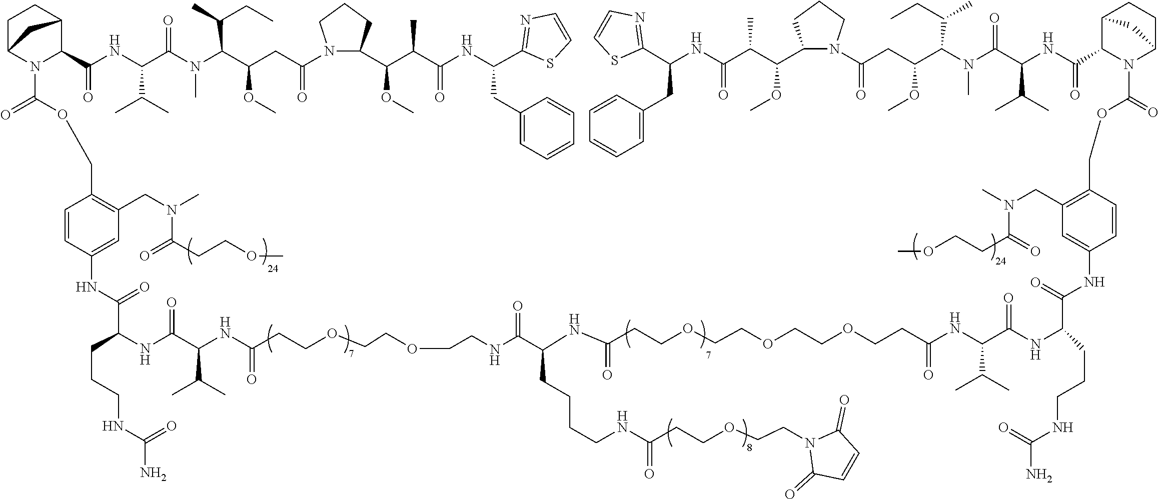 |
| P4-L8- P3 | 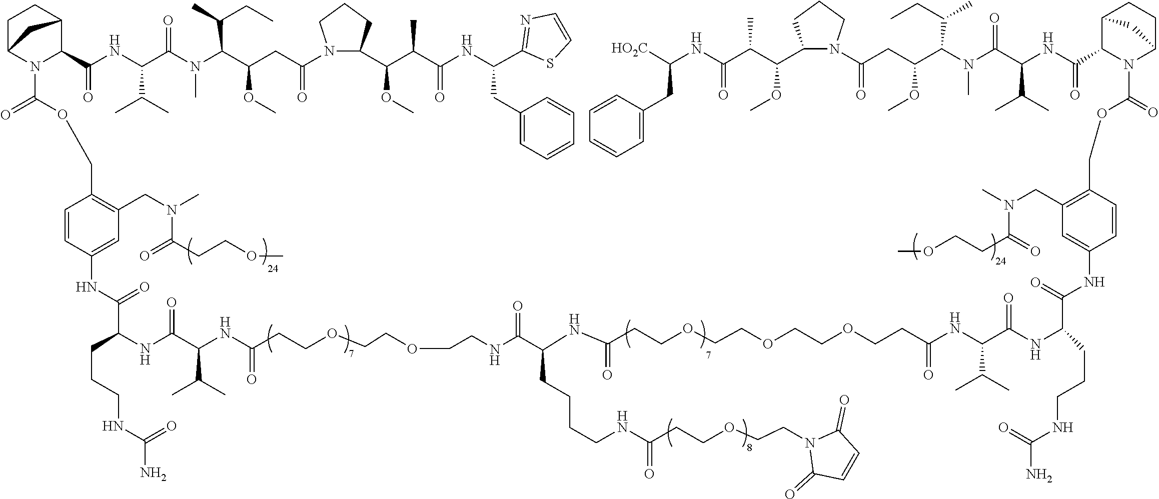 |
| P4-L8- P4 | 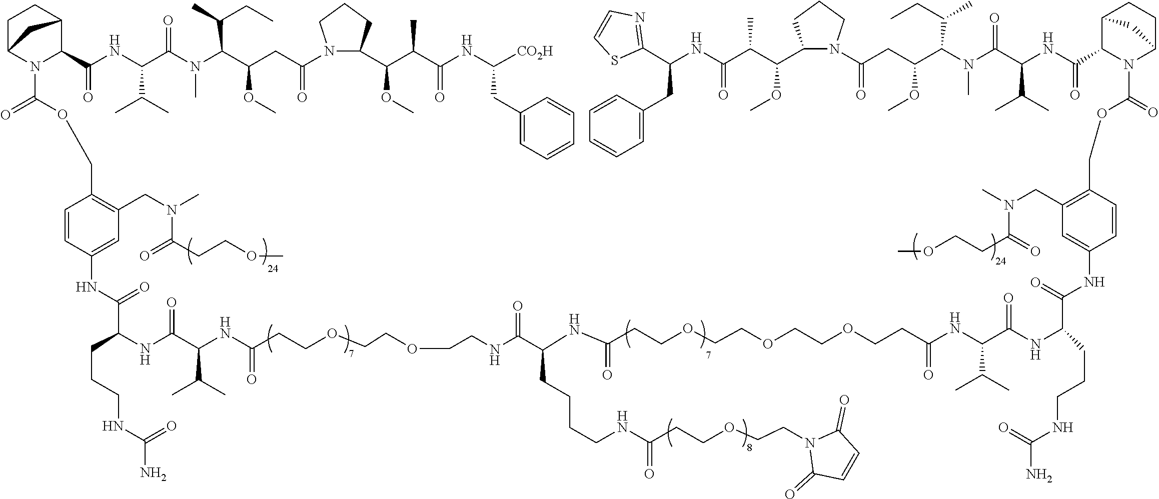 |
| P5-L8- P5 | 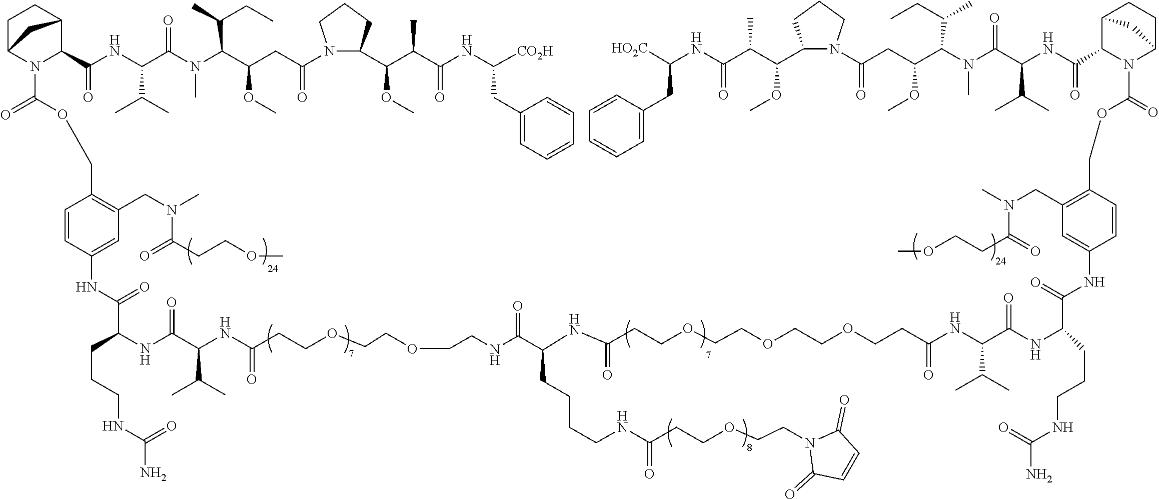 |
| P5-L8- P7 | 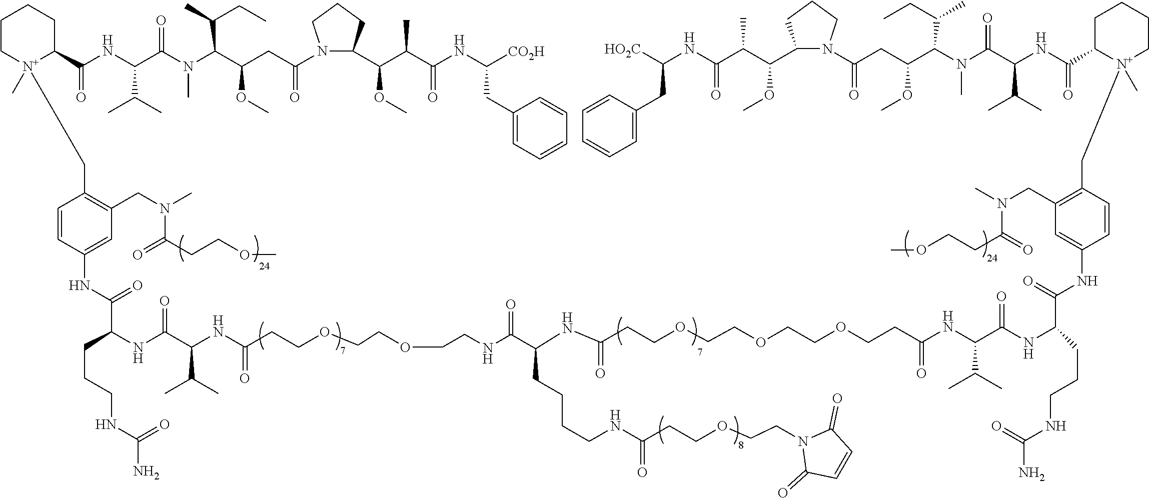 |
| P5-L8- P8 | 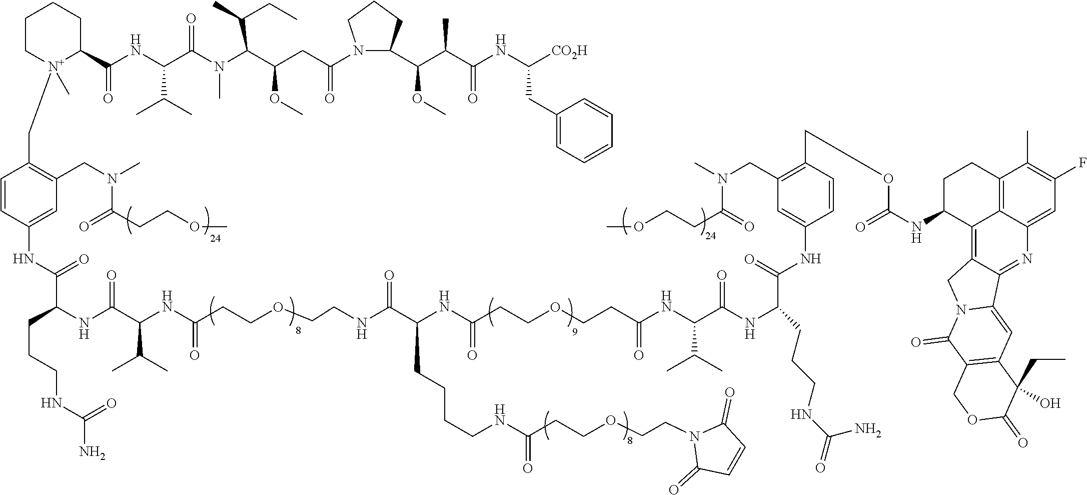 |
| P6-L8- P6 | 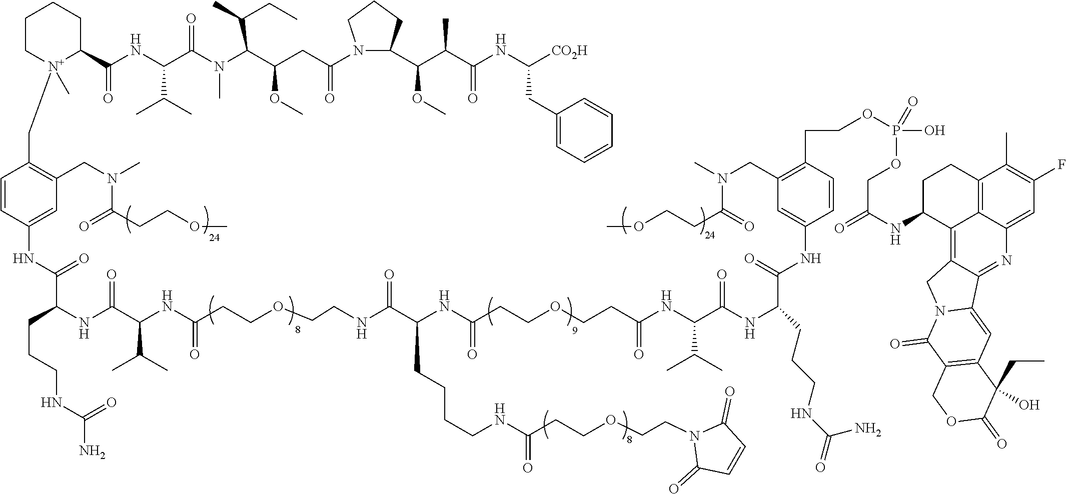 |
| P7-L8- P7 | 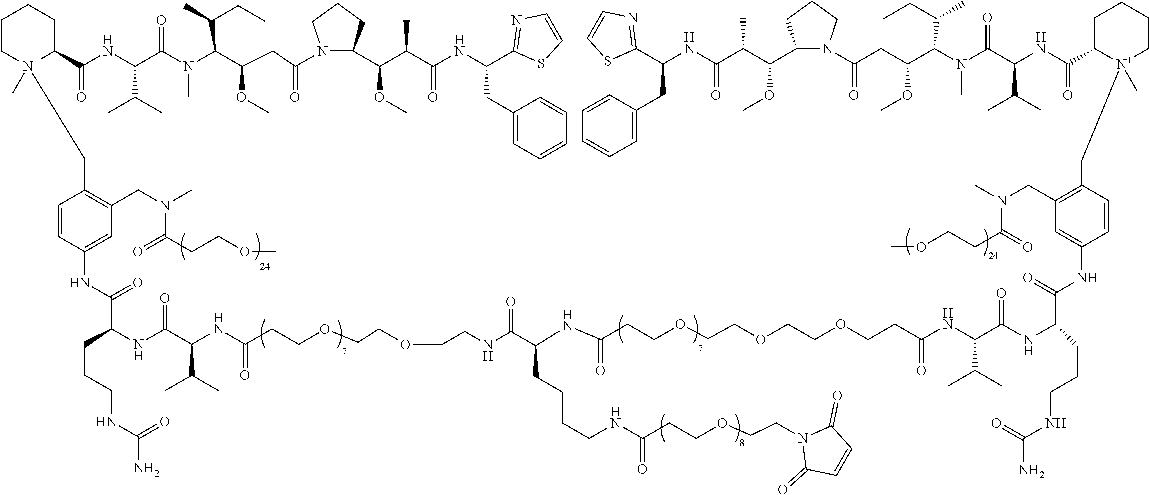 |
| P8-L8- P8 | 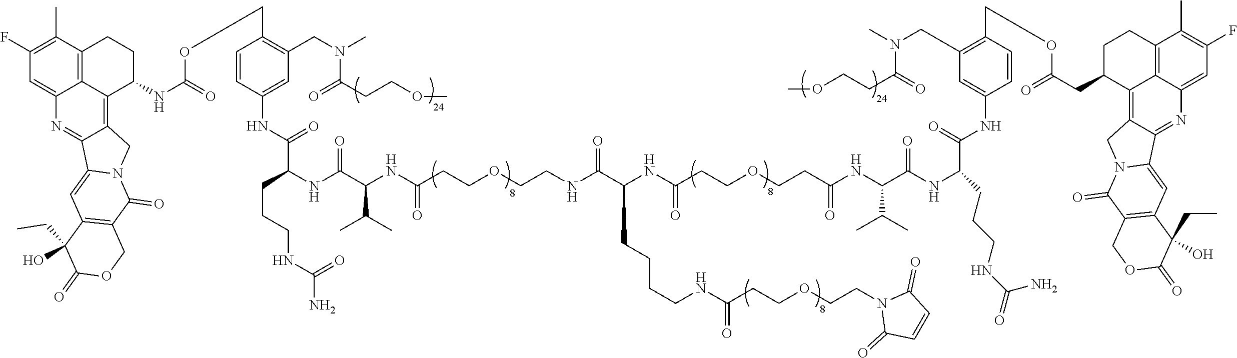 |
| P1-L9- P1 | 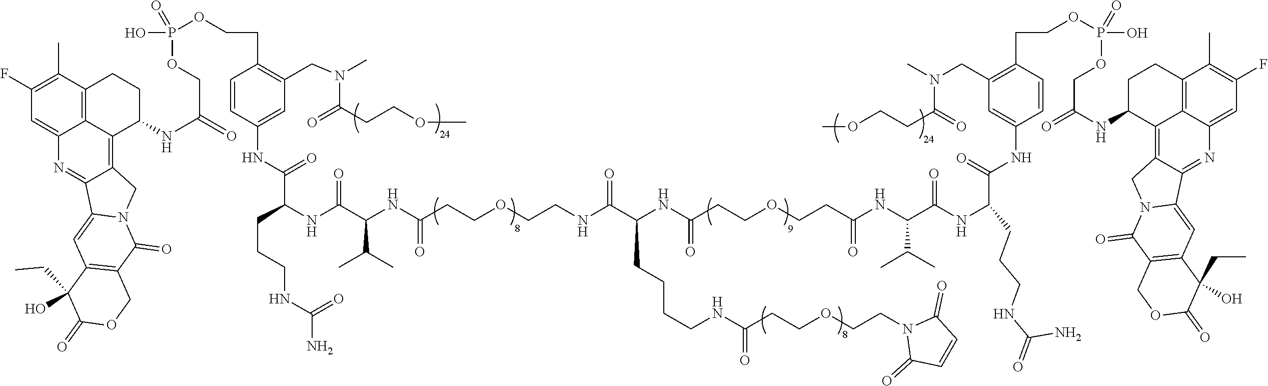 |
| P2-L9- P1 | 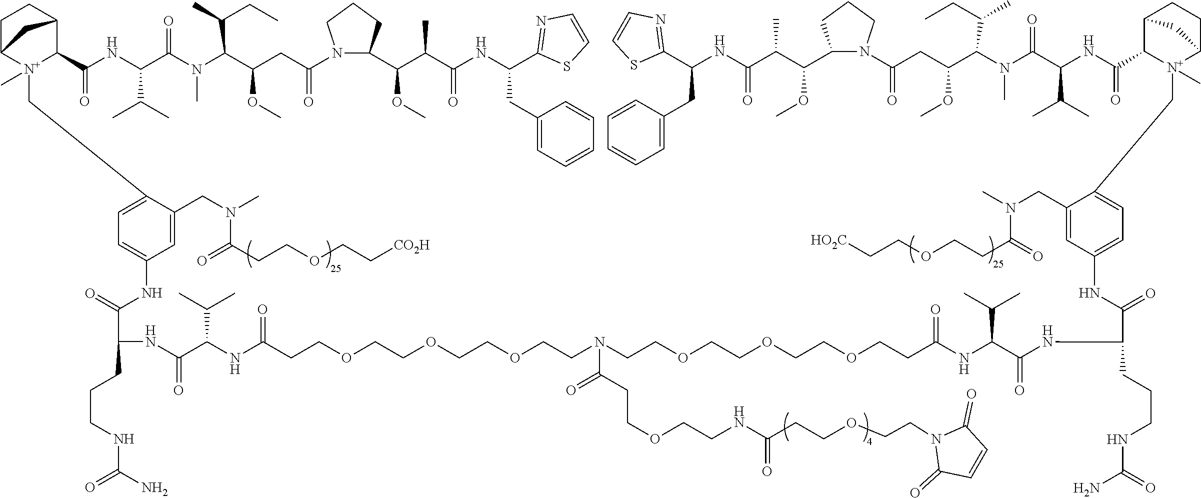 |
| P2-L9- P2 | 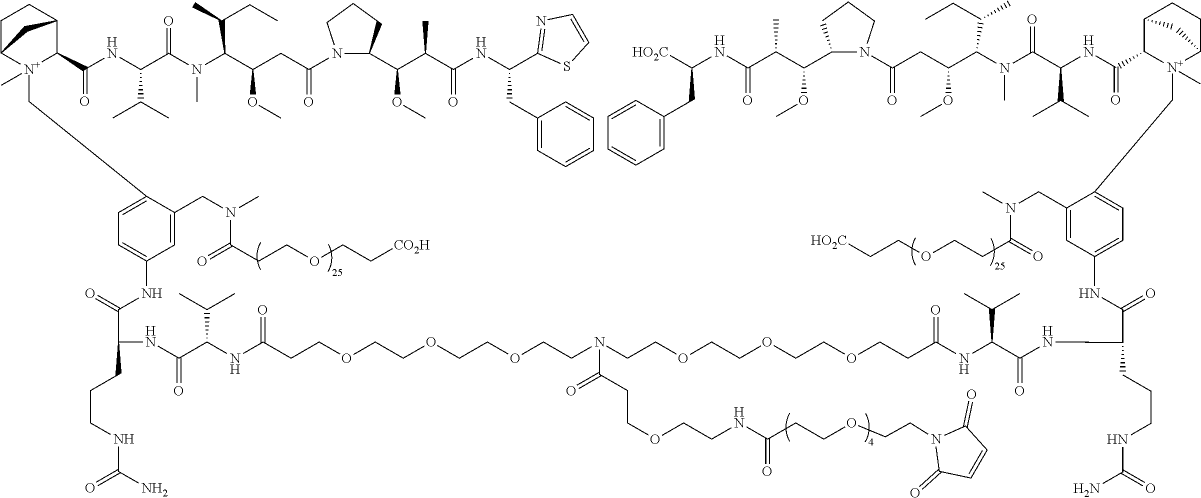 |
| P3-L9- P3 | 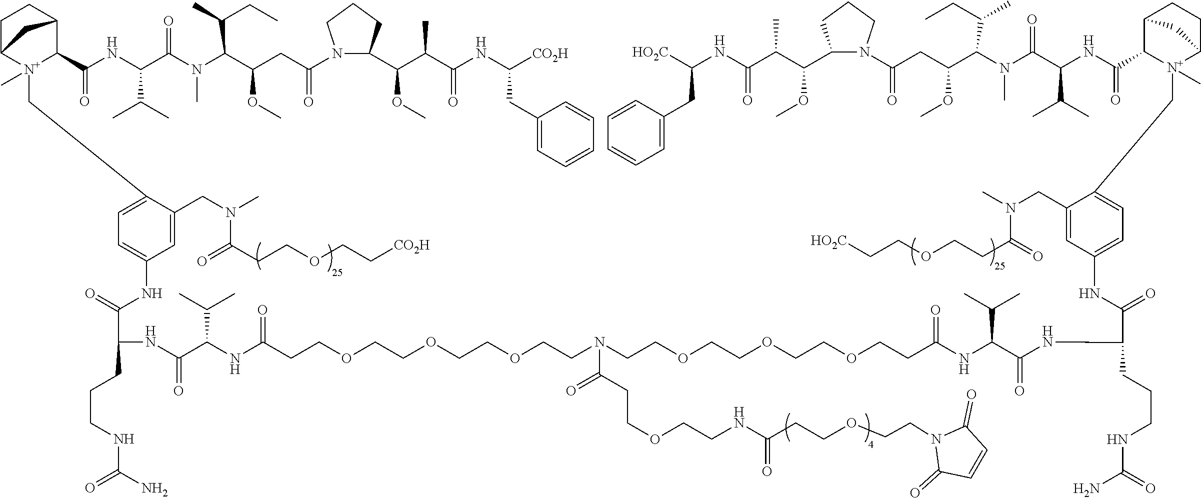 |
| P3-L9- P4 | 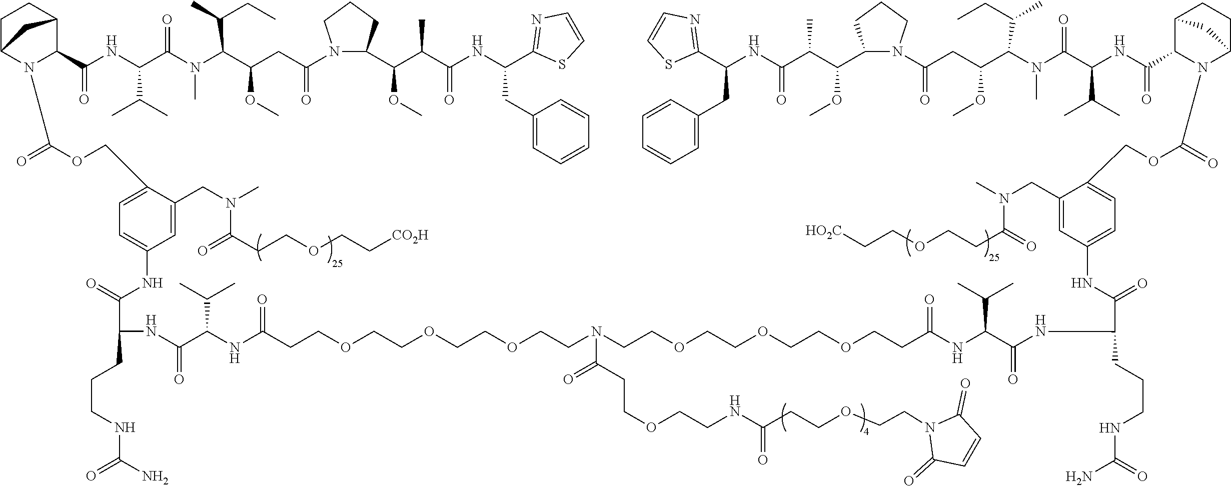 |
| P4-L9- P4 | 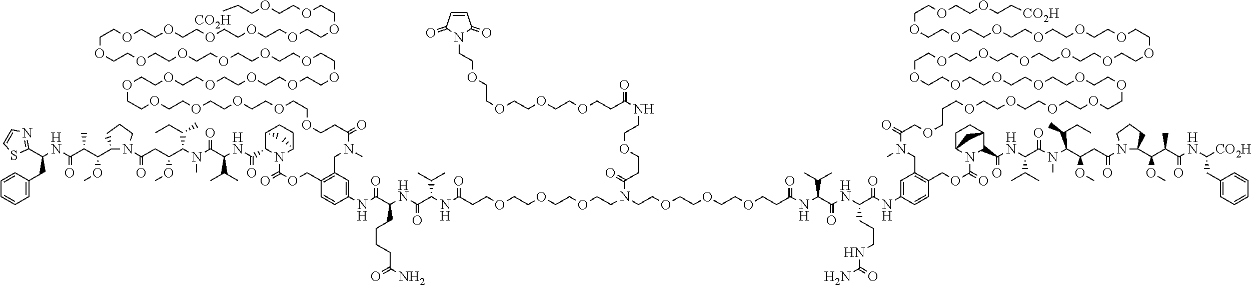 |
| P5-L9- P5 | 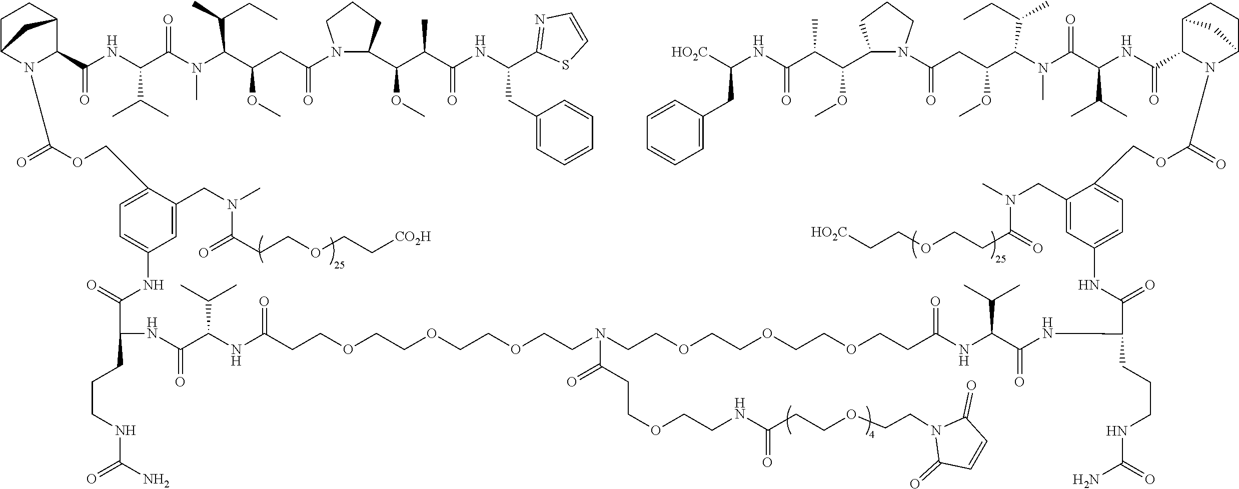 |
| P6-L9- P6 | 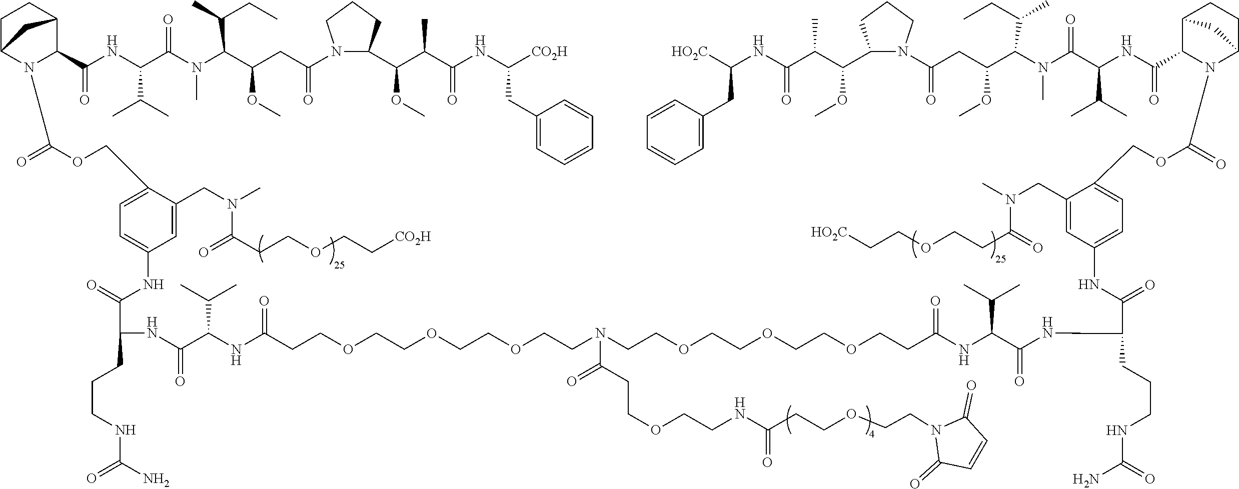 |
| P7-L9- P7 | 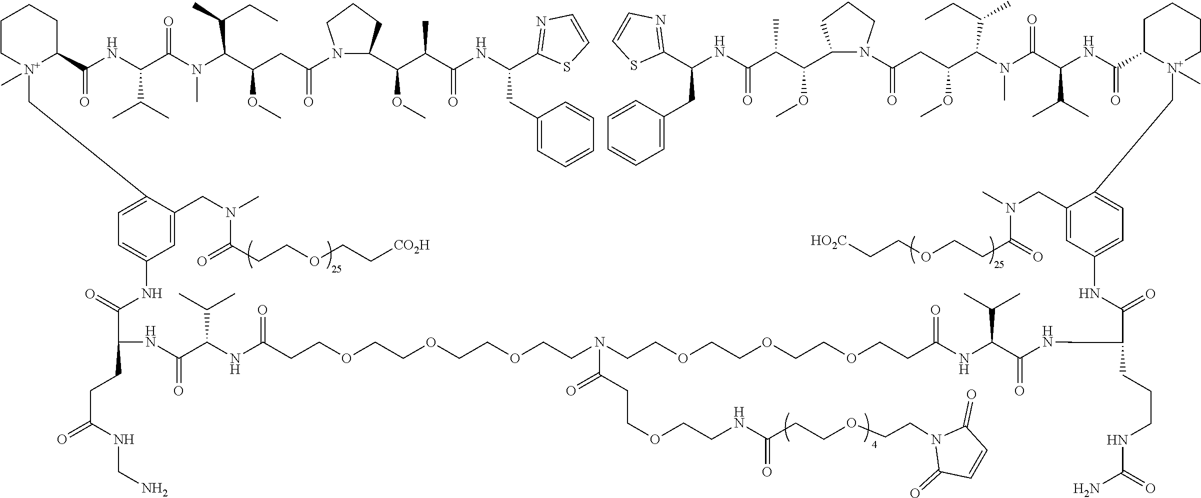 |
| P8-L9- P8 | 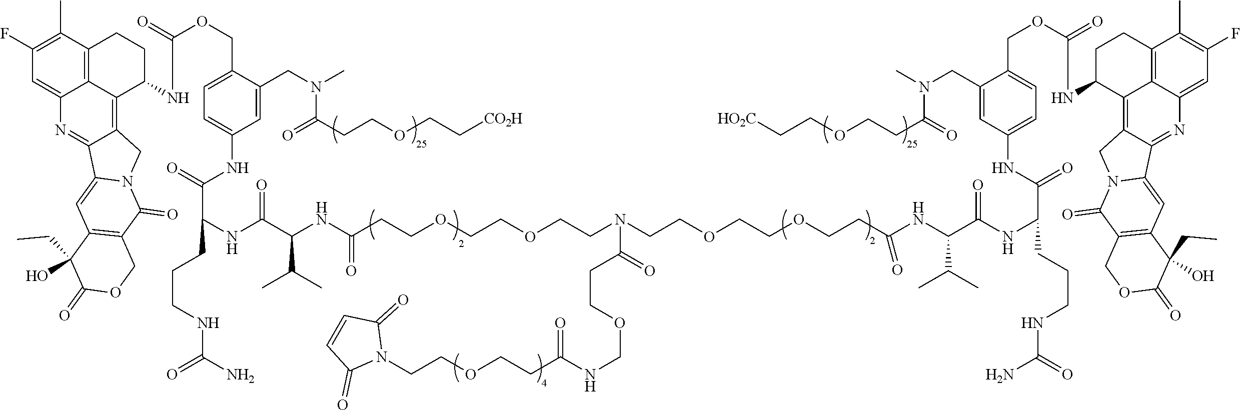 |
| P1- L10-P1 | 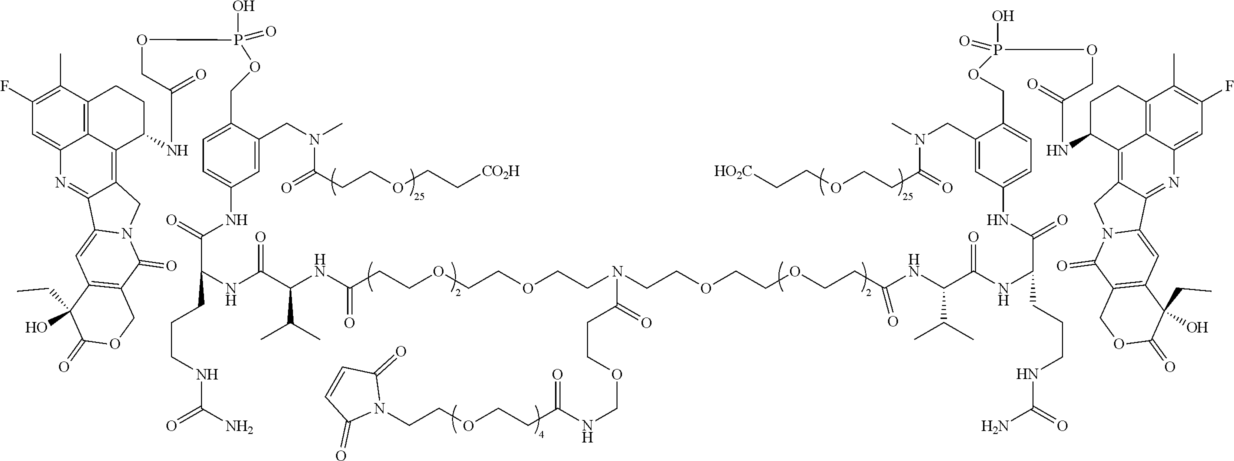 |
| P1- L10-P2 | 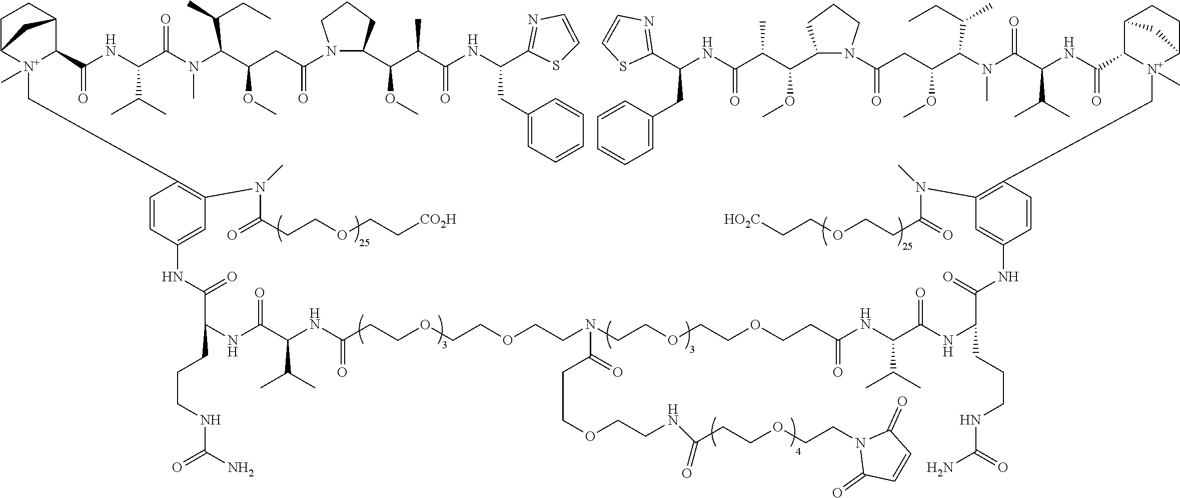 |
| P2- L10-P2 | 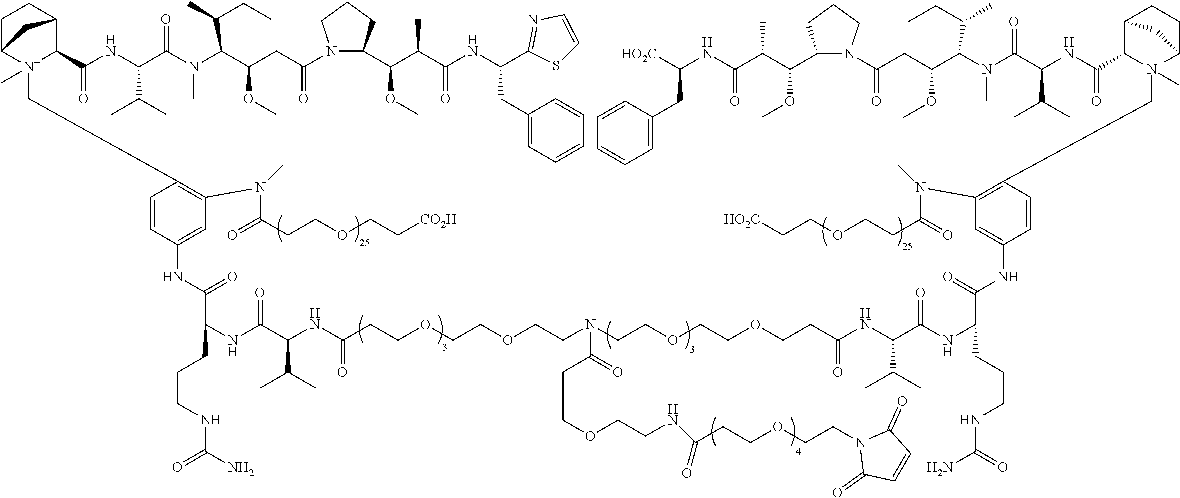 |
| P3- L10-P3 | 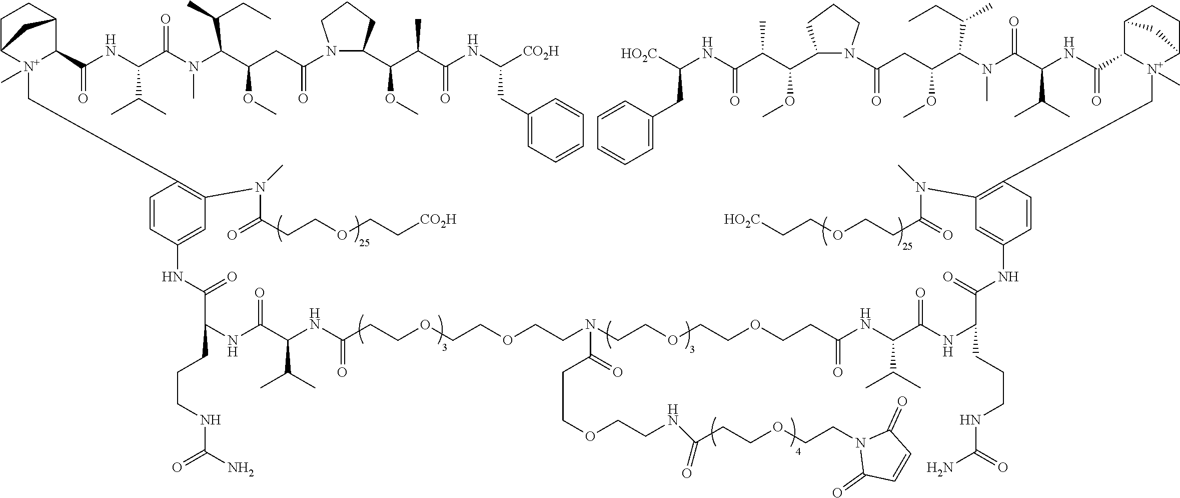 |
| P3- L10-P4 | 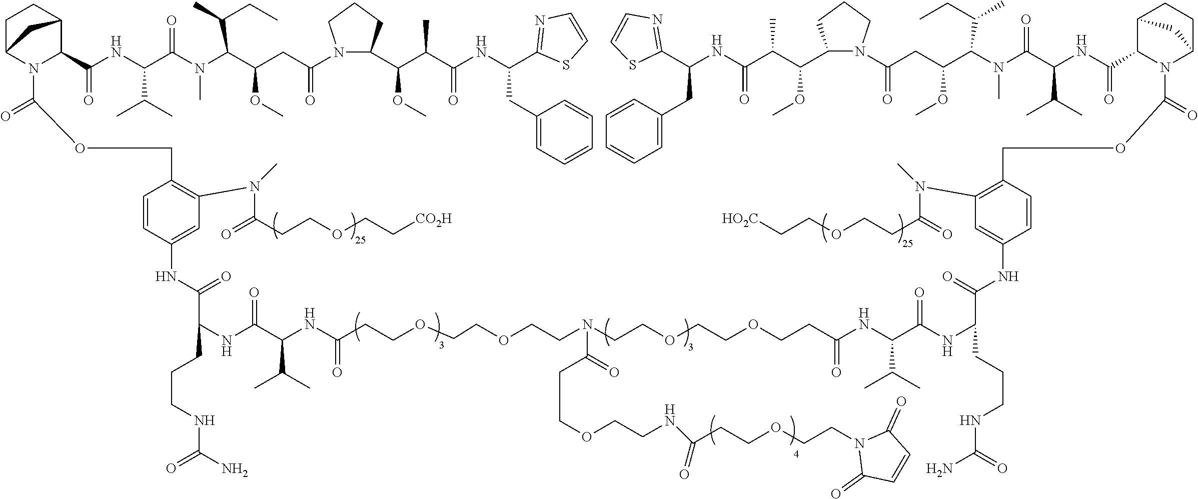 |
| P4- L10-P4 | 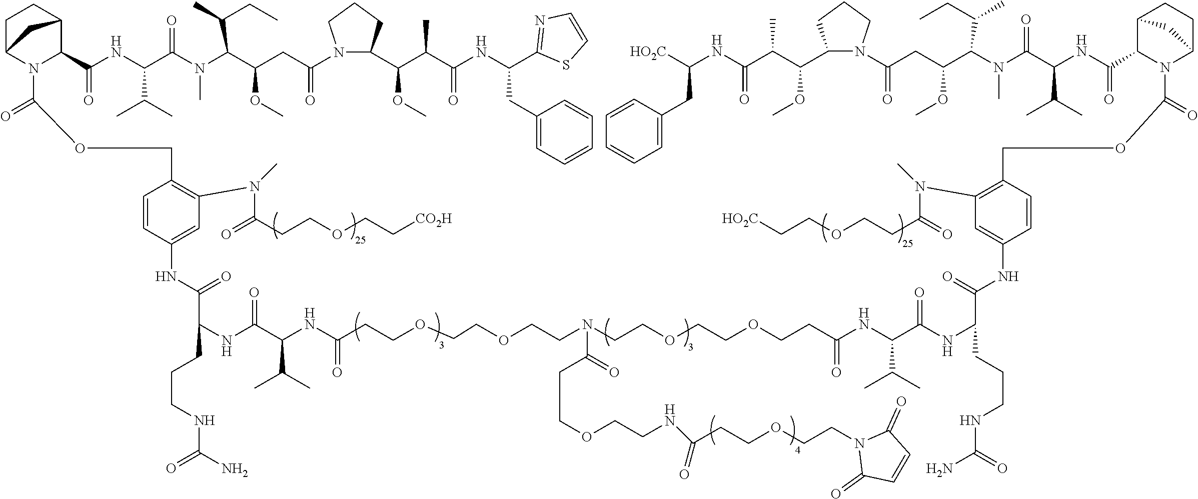 |
| P5- L10-P5 | 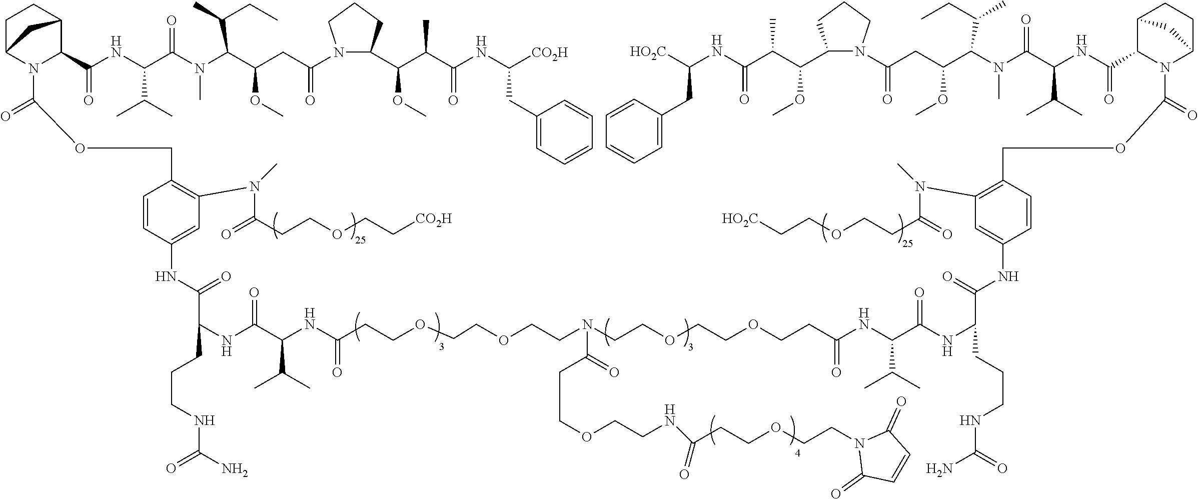 |
| P6- L10-P6 | 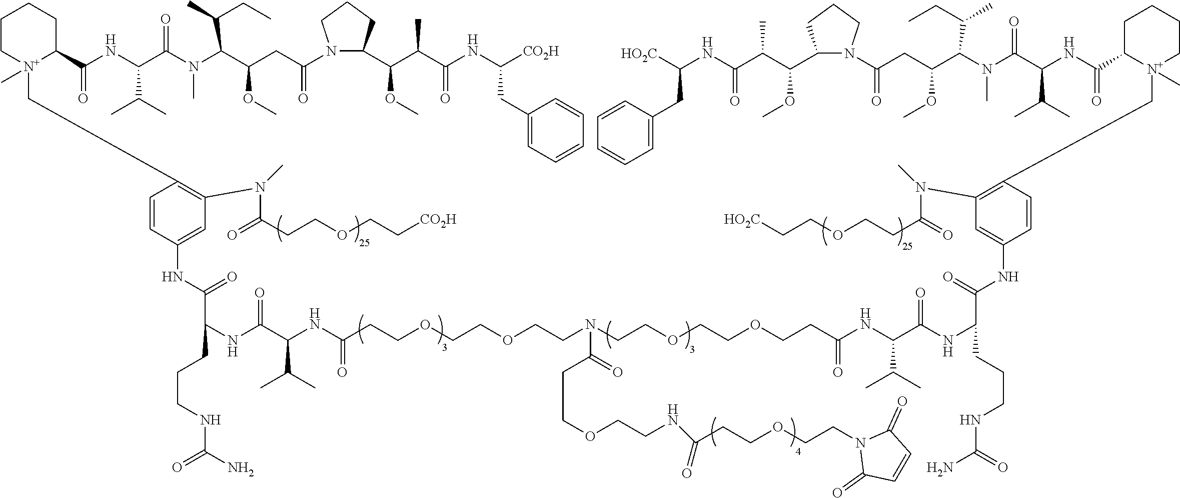 |
| P7- L10-P7 | 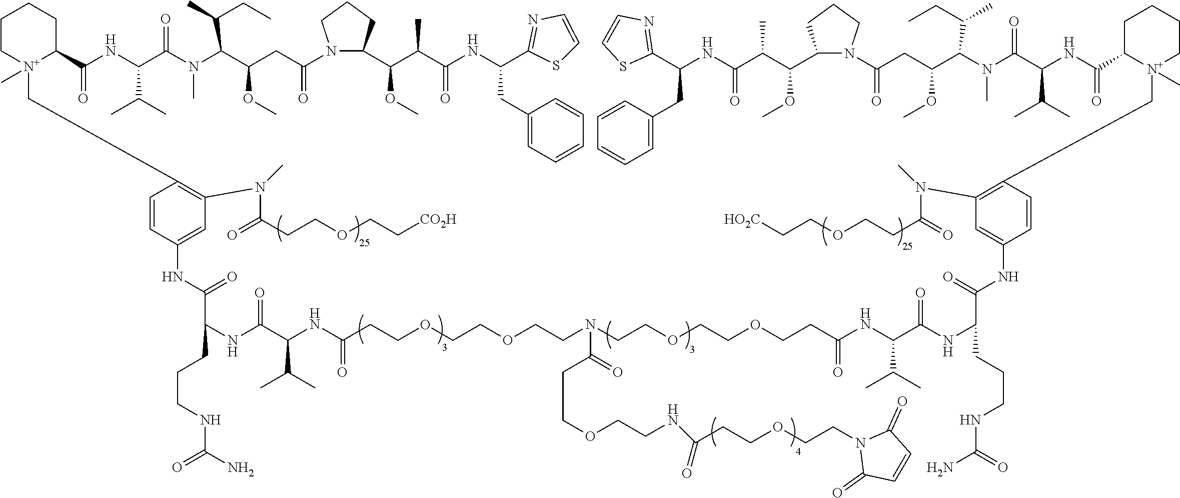 |
| P8- L10-P8 | 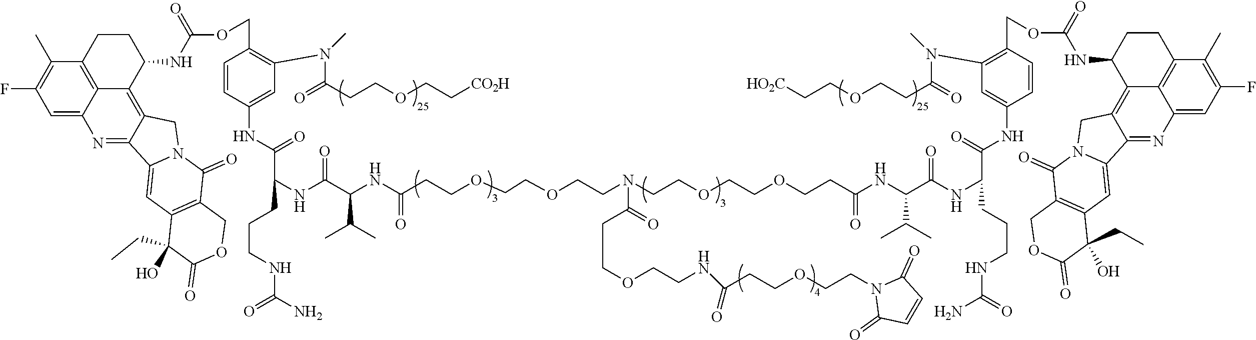 |
| P1- L11-P1 | 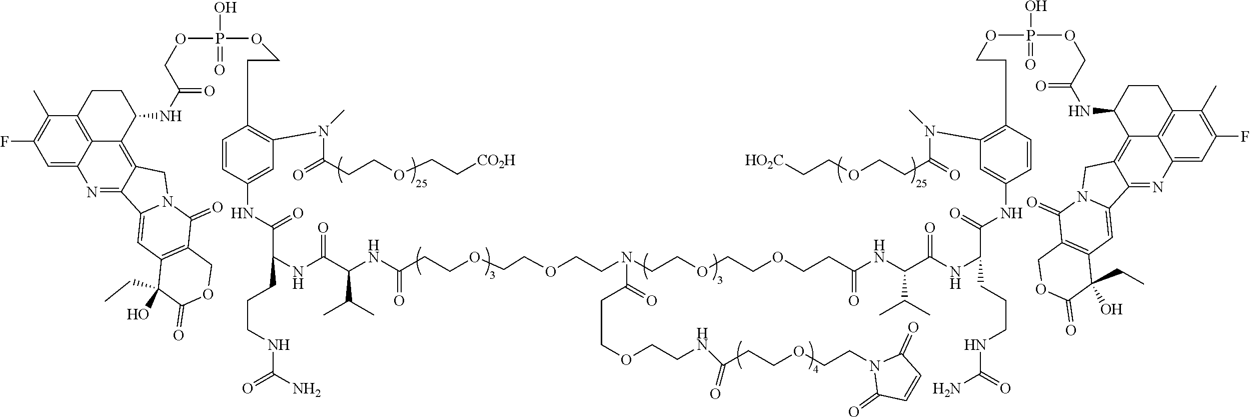 |
| P1- L11-P2 | 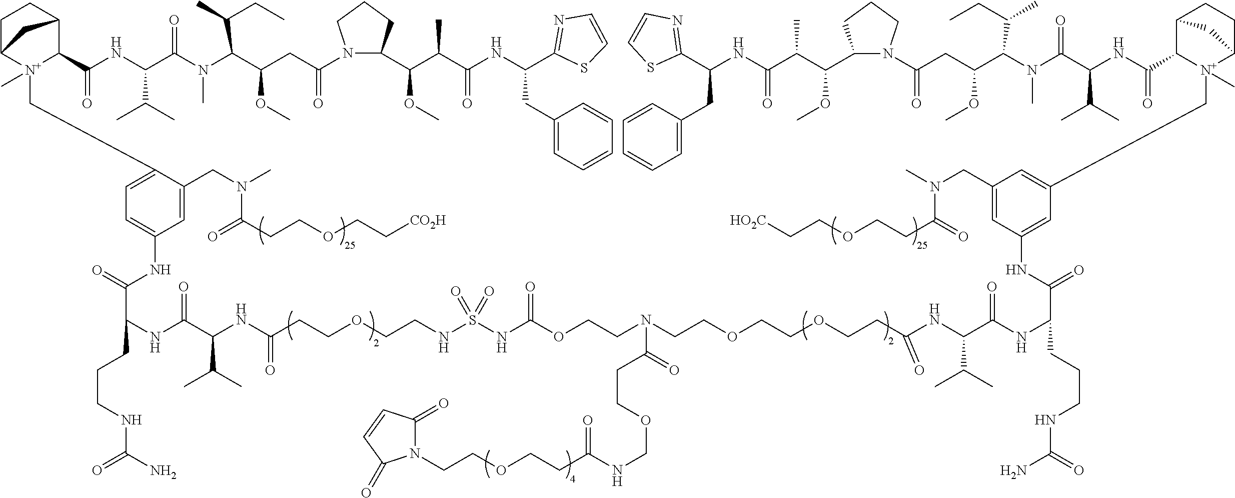 |
| P2- L11-P1 | 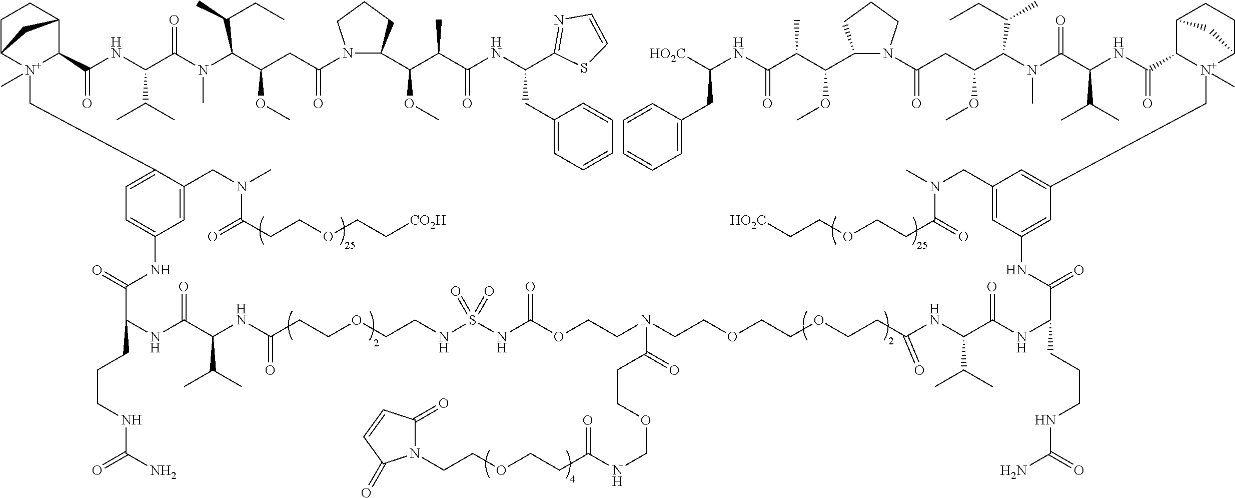 |
| P2- L11-P2 | 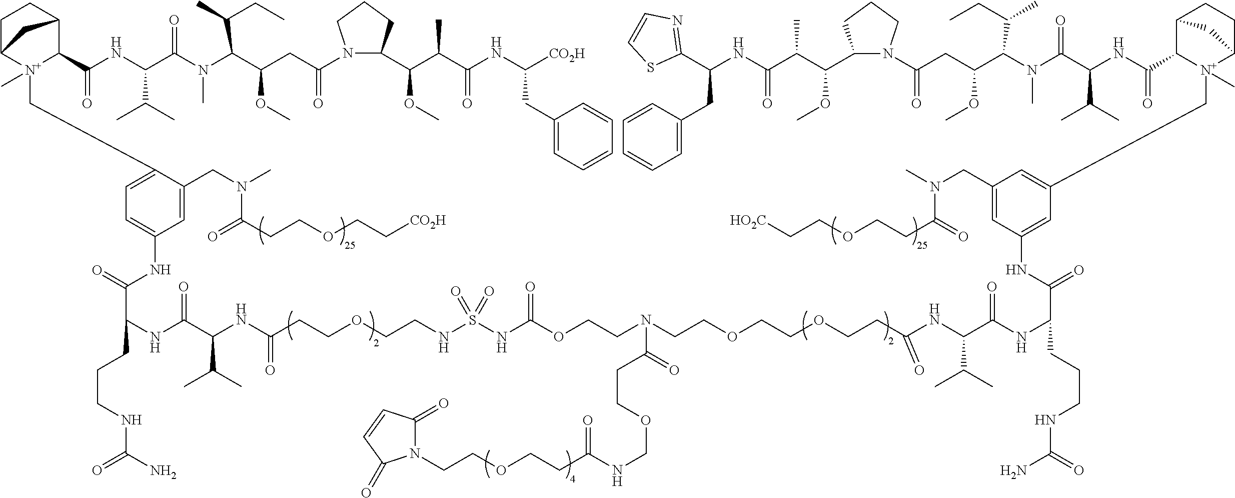 |
| P3- L11-P3 | 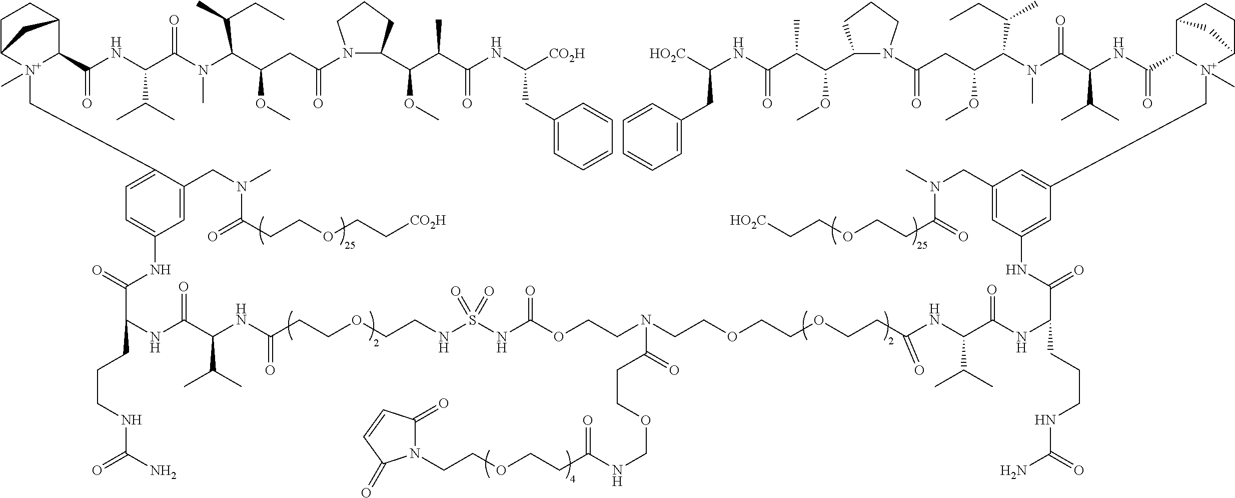 |
| P3- L11-P4 | 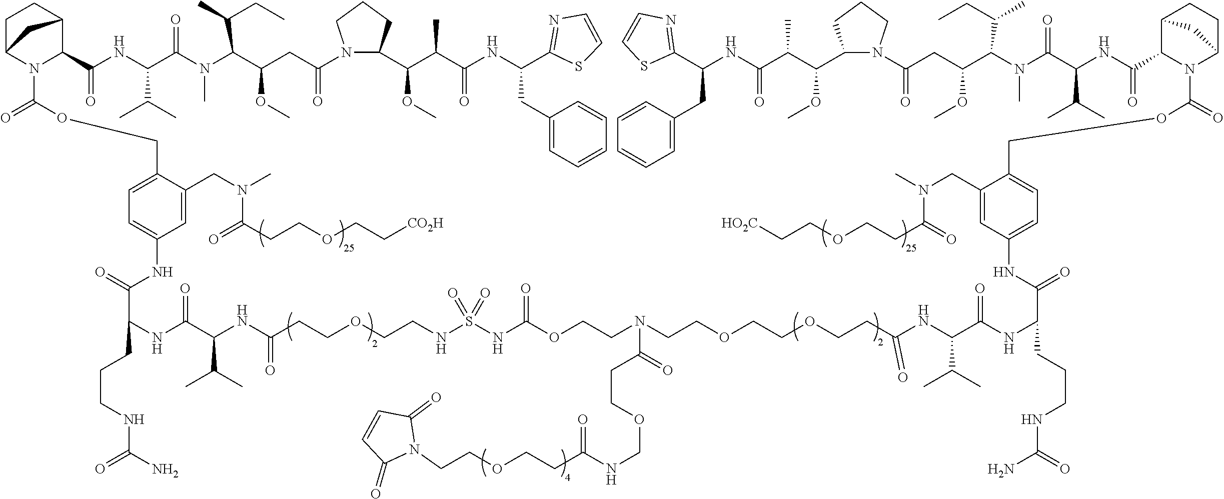 |
| P4- L11-P3 | 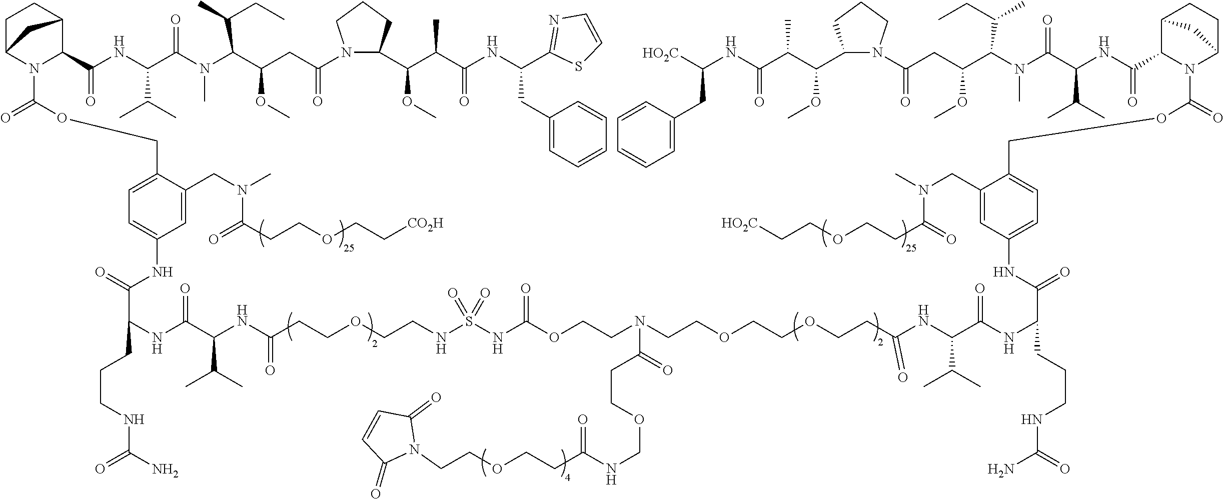 |
| P4- L11-P4 | 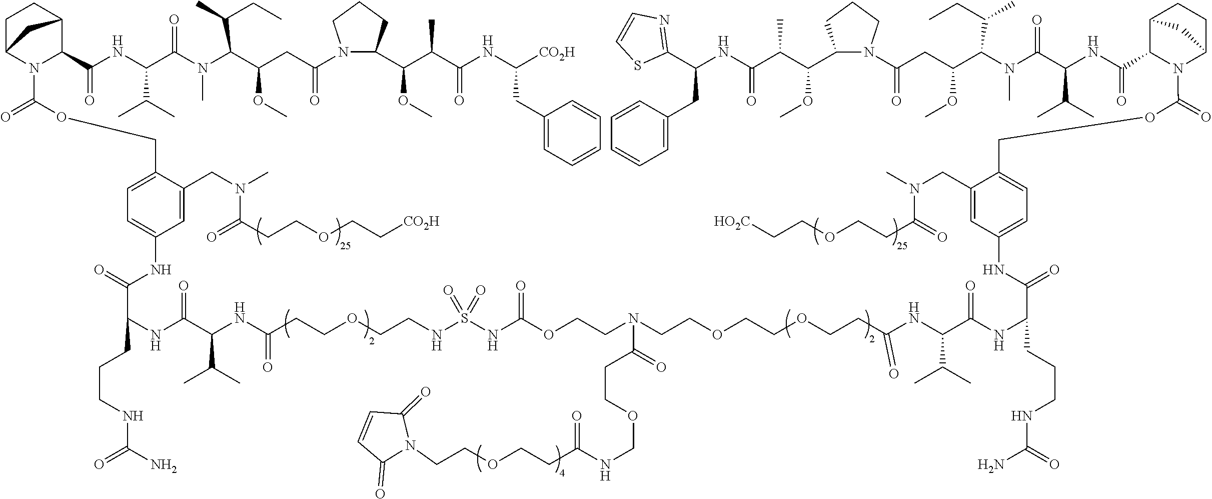 |
| P1- L12-P1 | 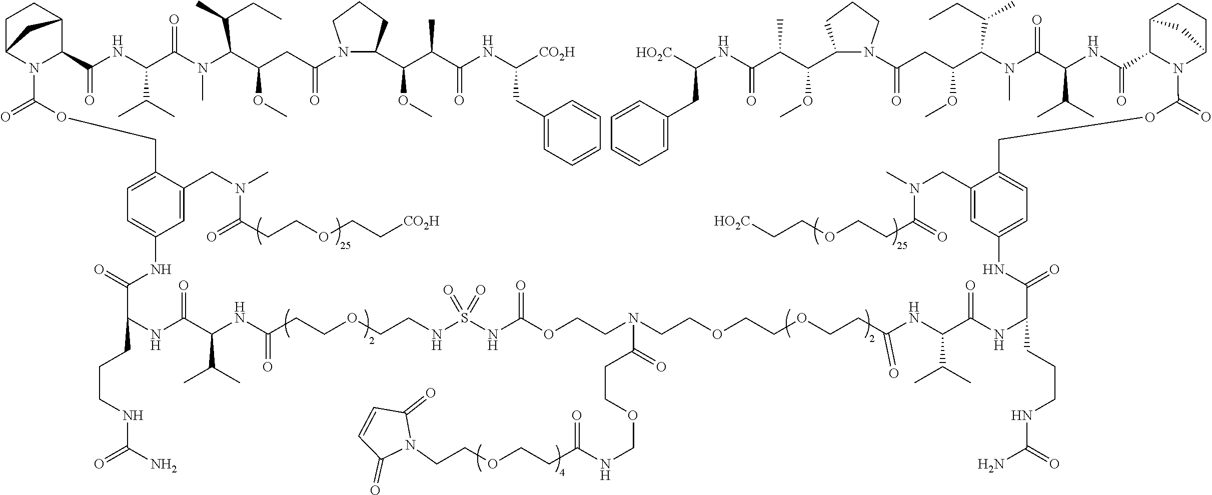 |
| P1- L12-P2 | 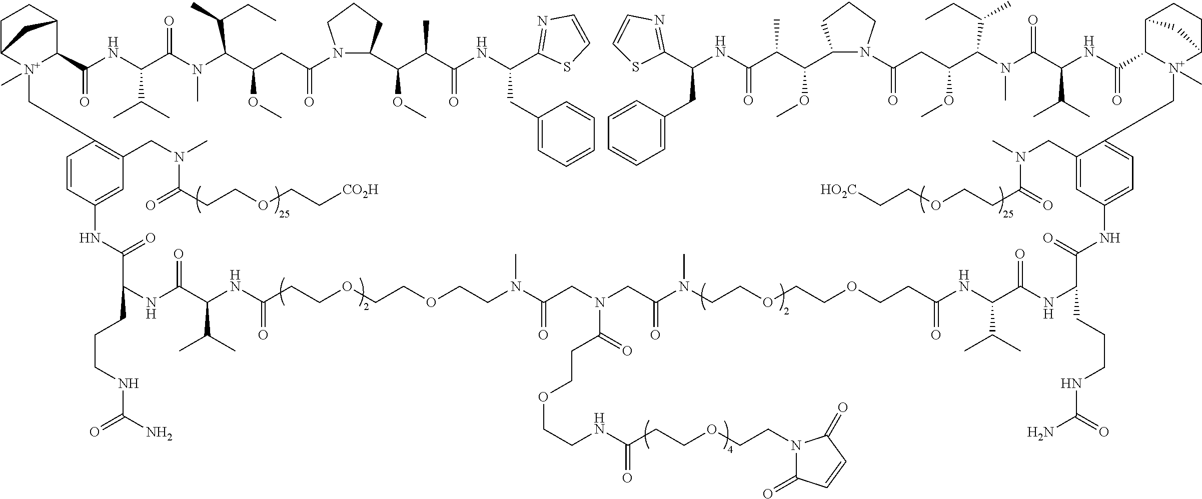 |
| P1- L12-P3 | 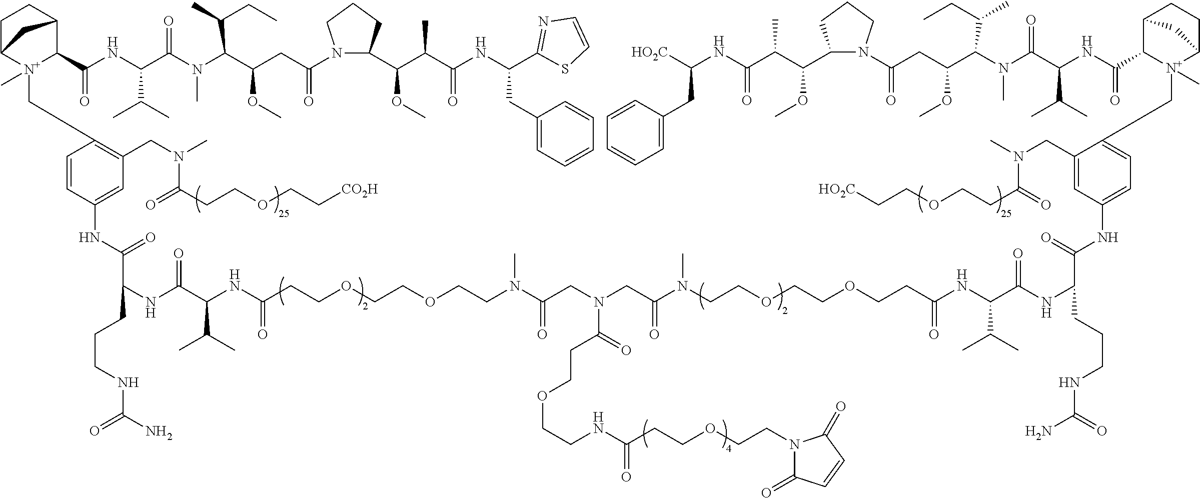 |
| P1- L12-P4 | 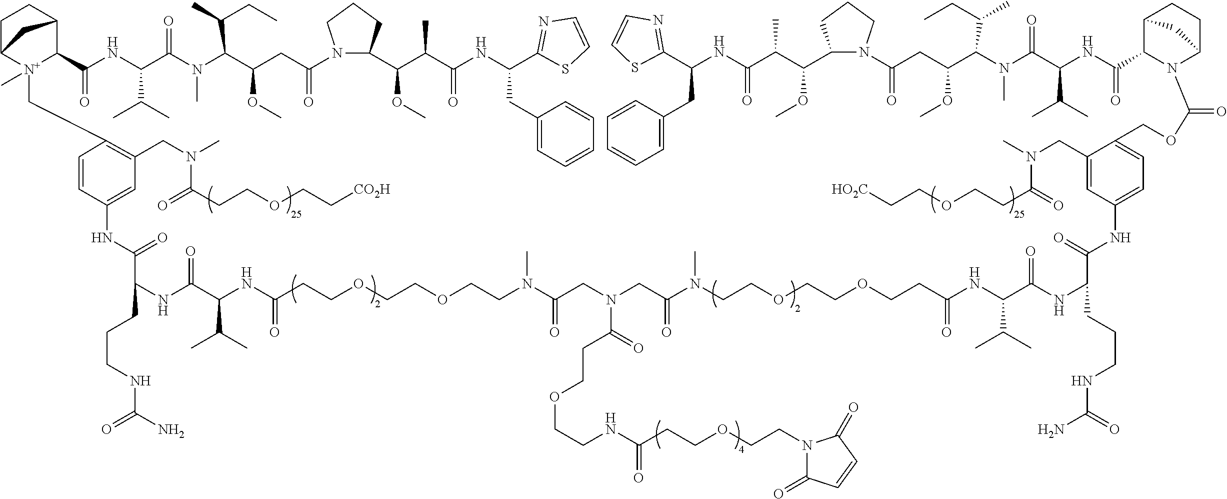 |
| P2- L12-P2 | 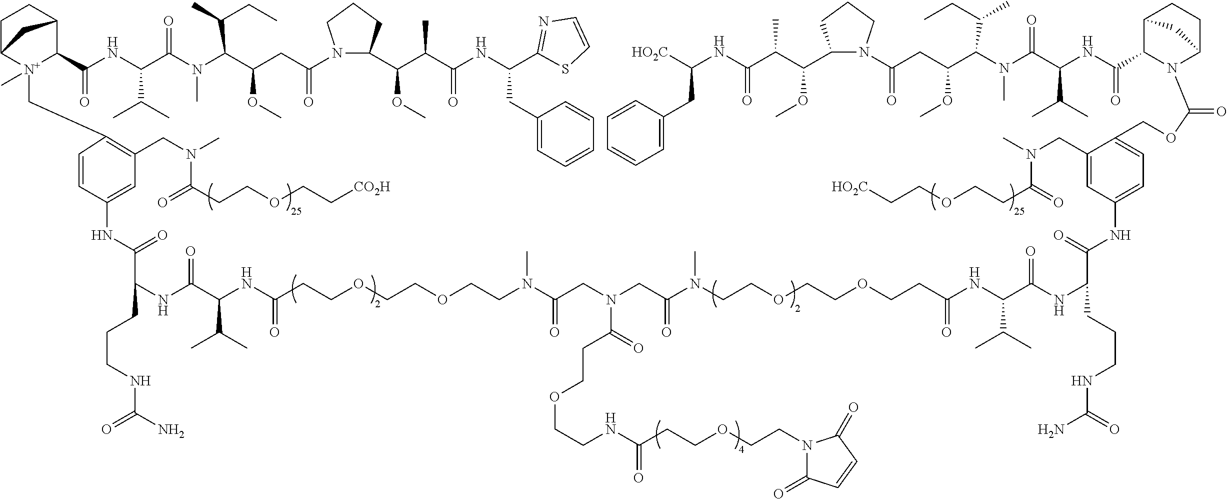 |
| P2- L12-P3 | 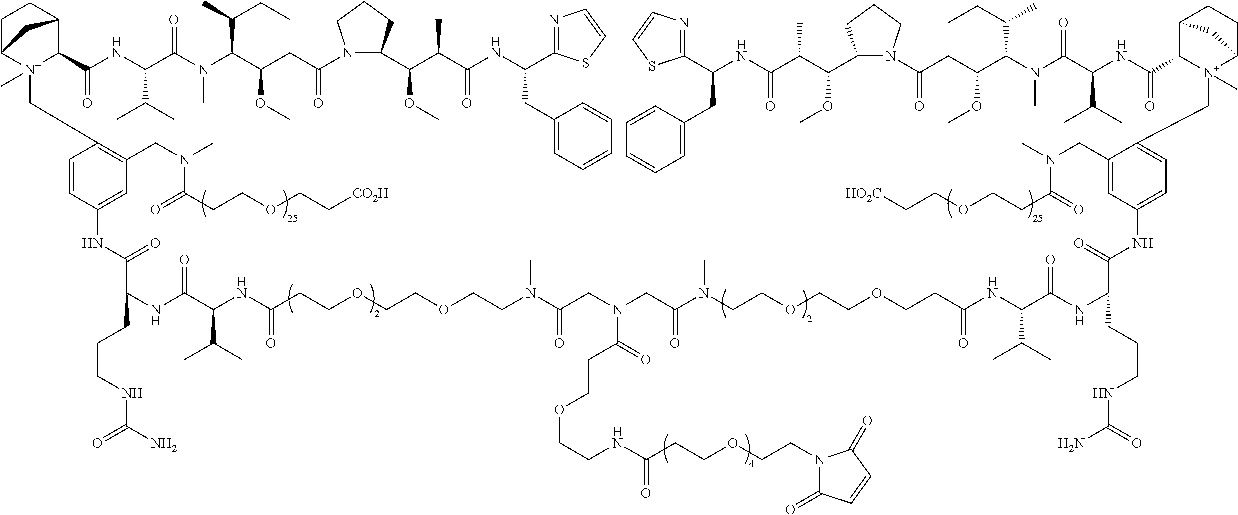 |
| P2- L12-P4 | 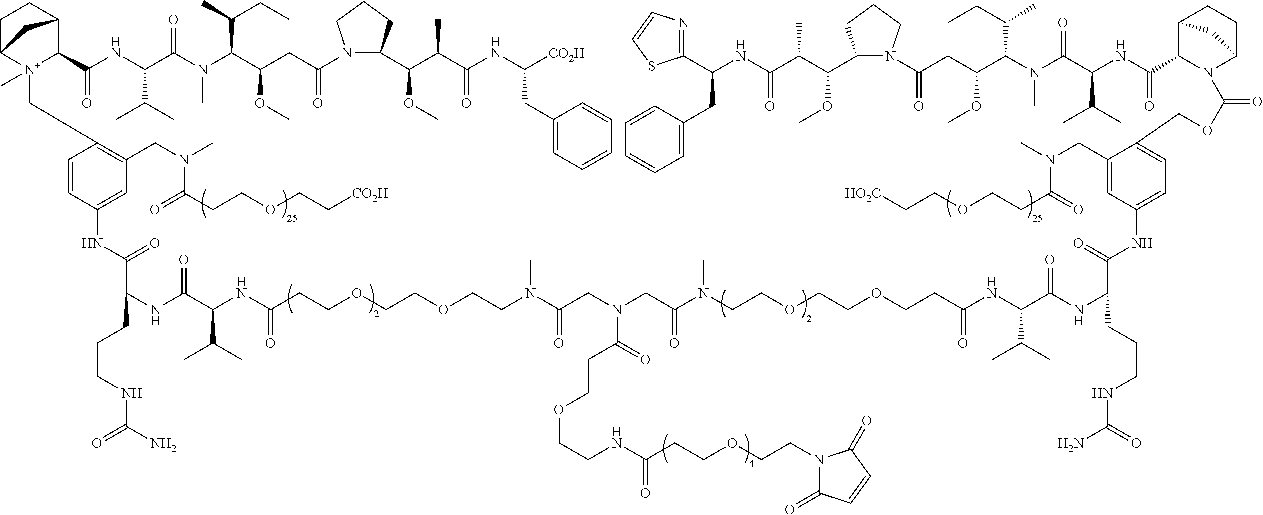 |
| P3- L12-P3 | 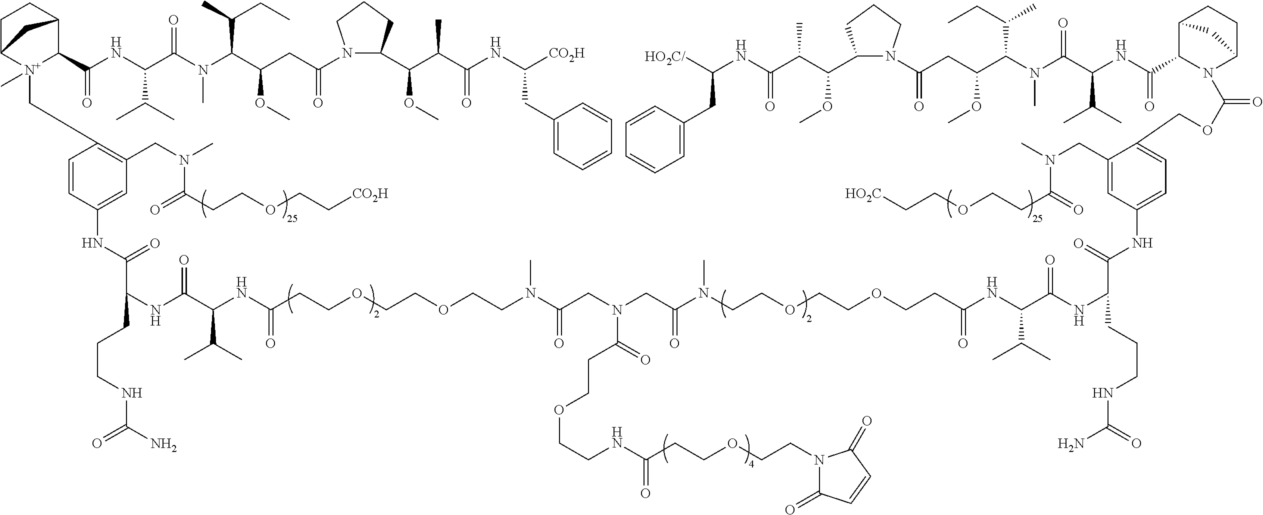 |
| P3- L12-P4 | 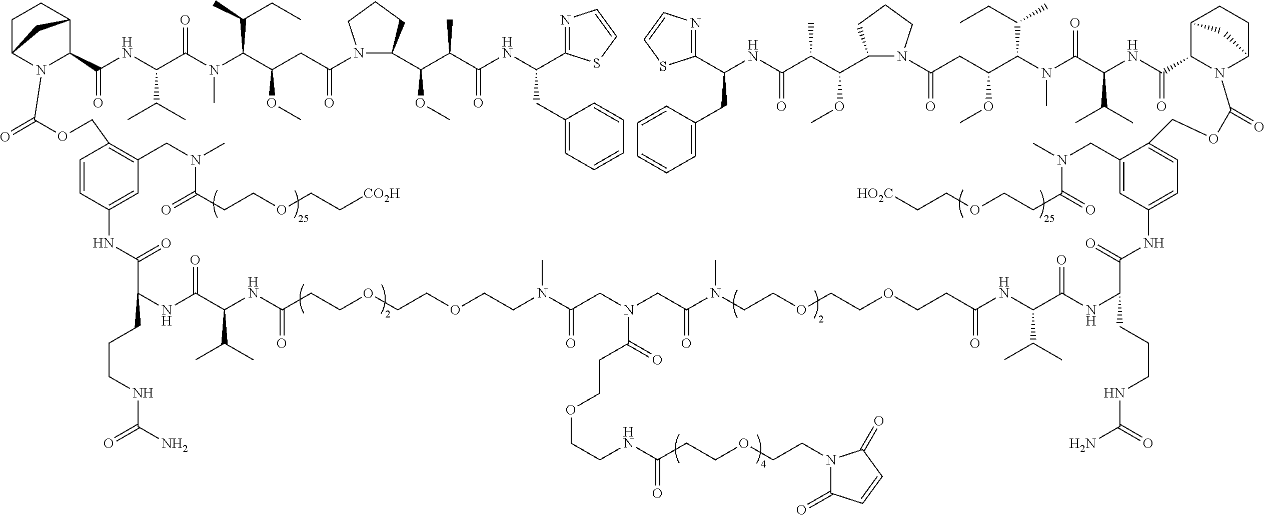 |
| P4- L12- P4 | 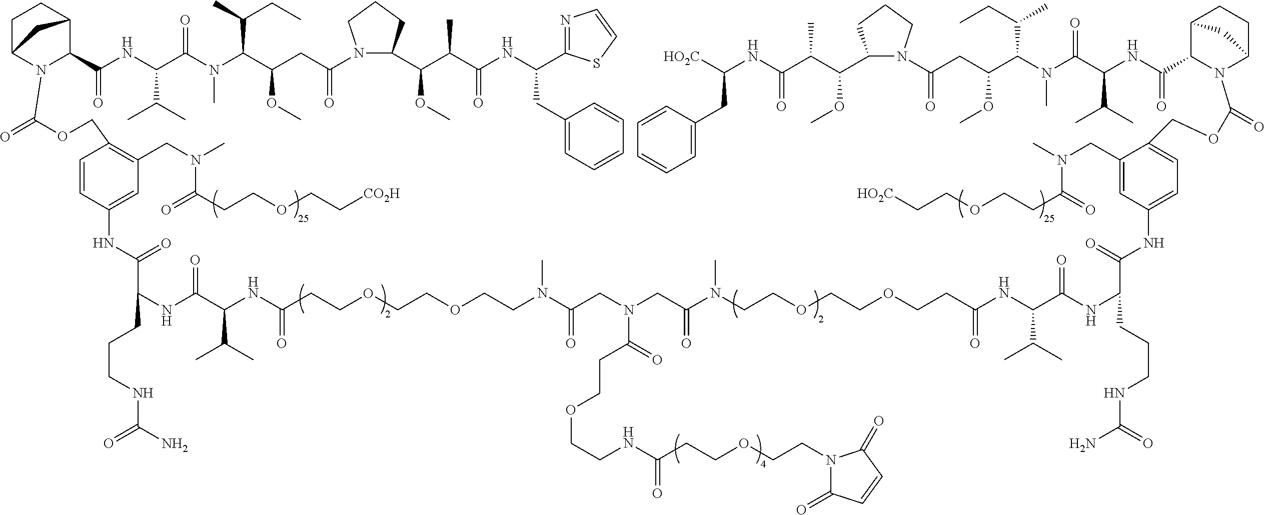 |
| P2- L12-P5 | 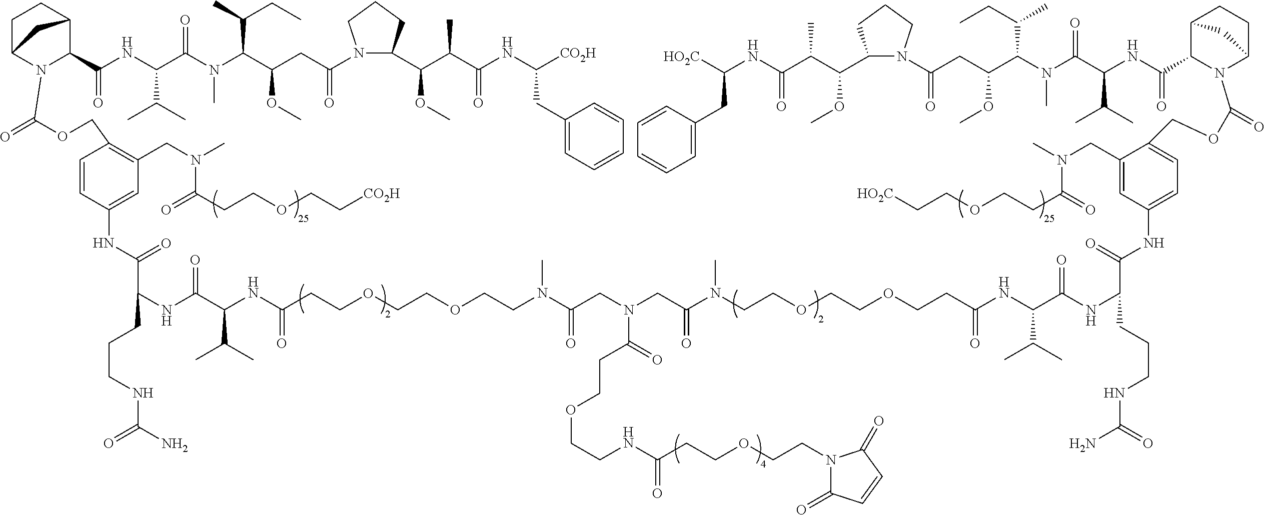 |
| P5- L12-P5 | 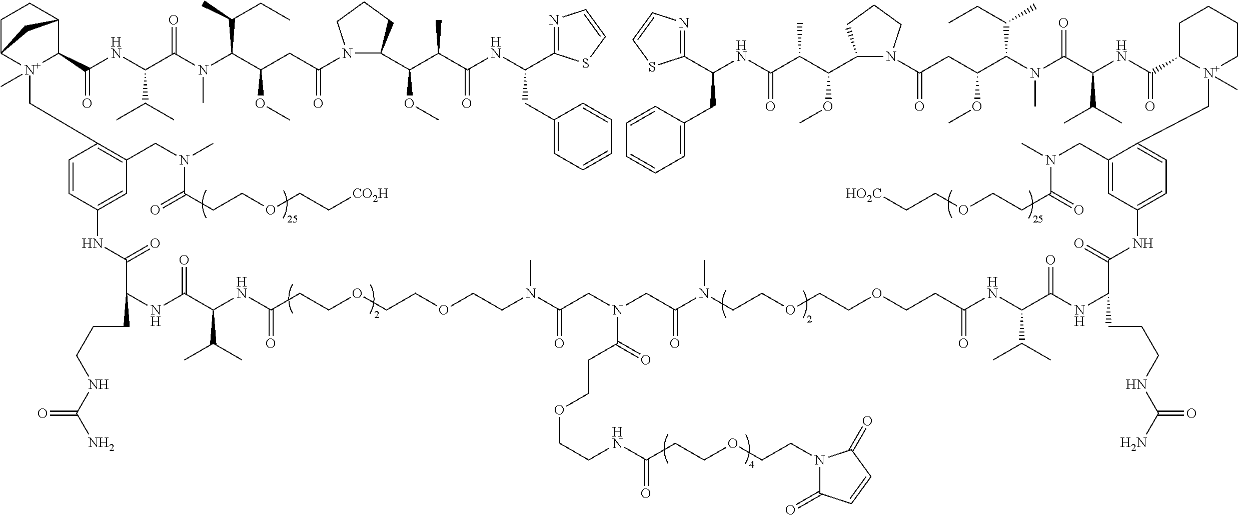 |
| P7- L12-P8 | 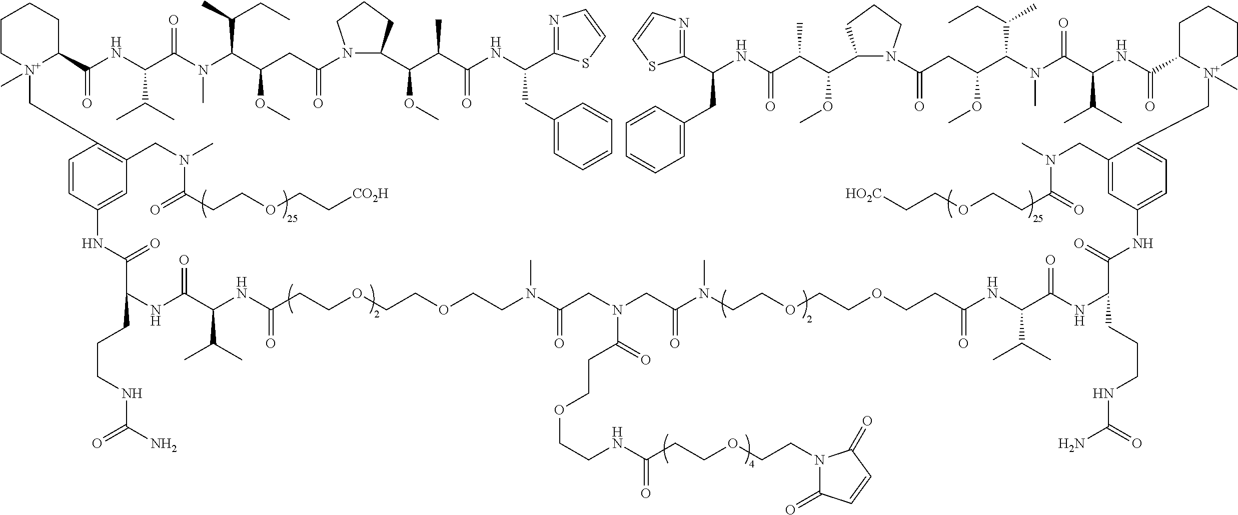 |
| P1- L13-P1 | 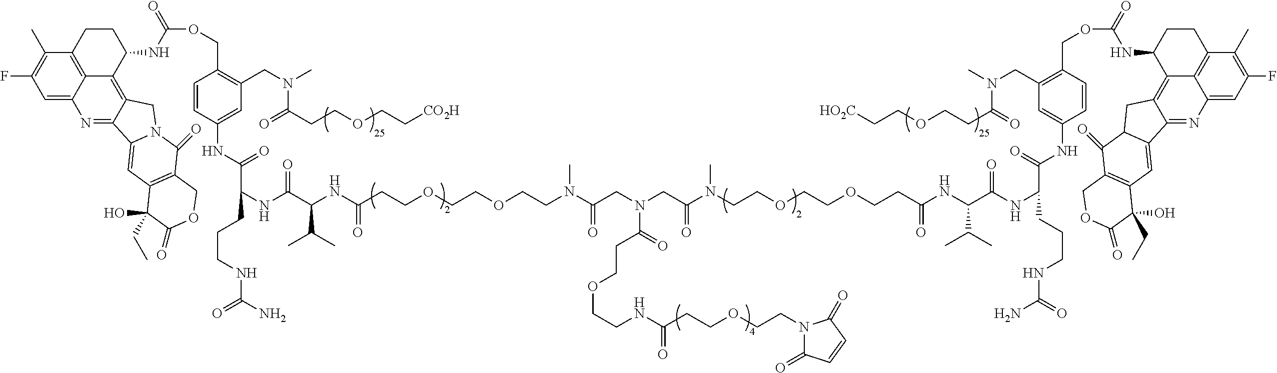 |
| P1- L13-P2 | 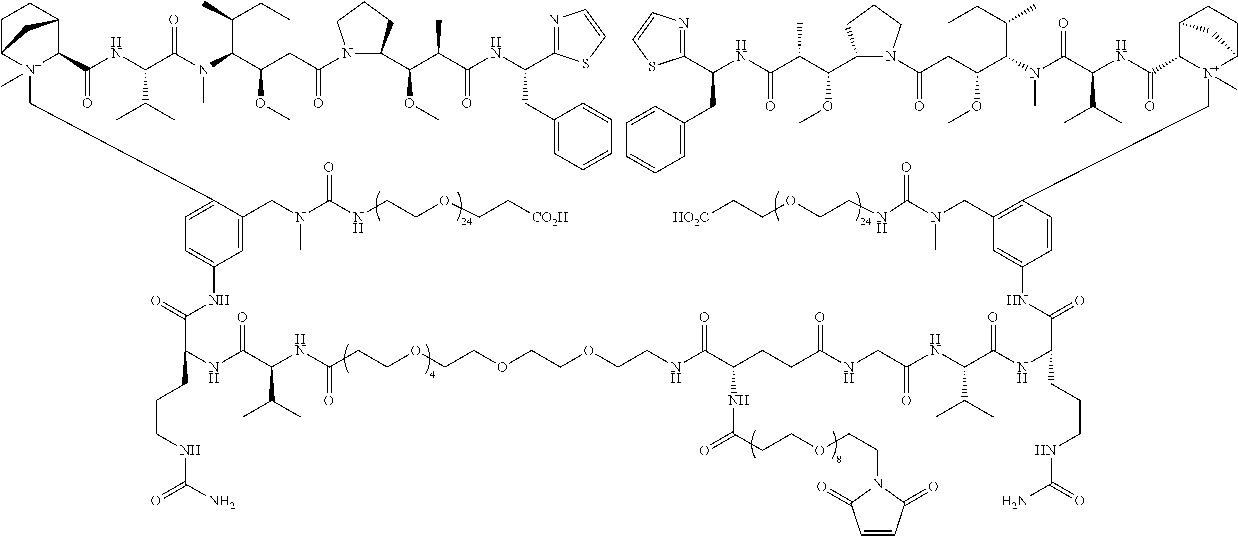 |
| P2- L13-P1 | 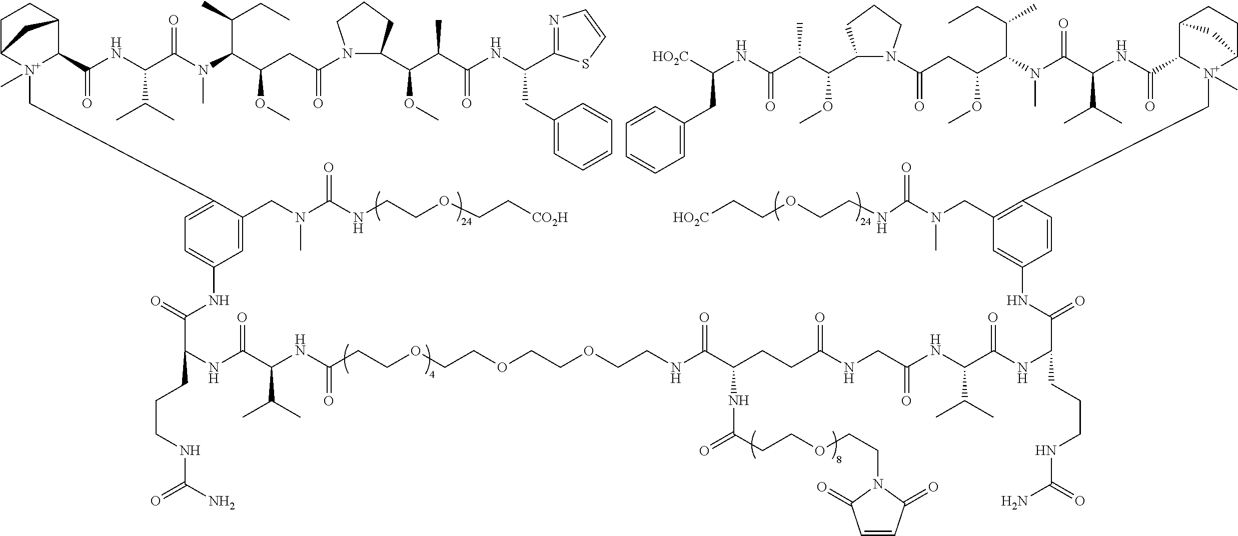 |
| P2- L13-P2 | 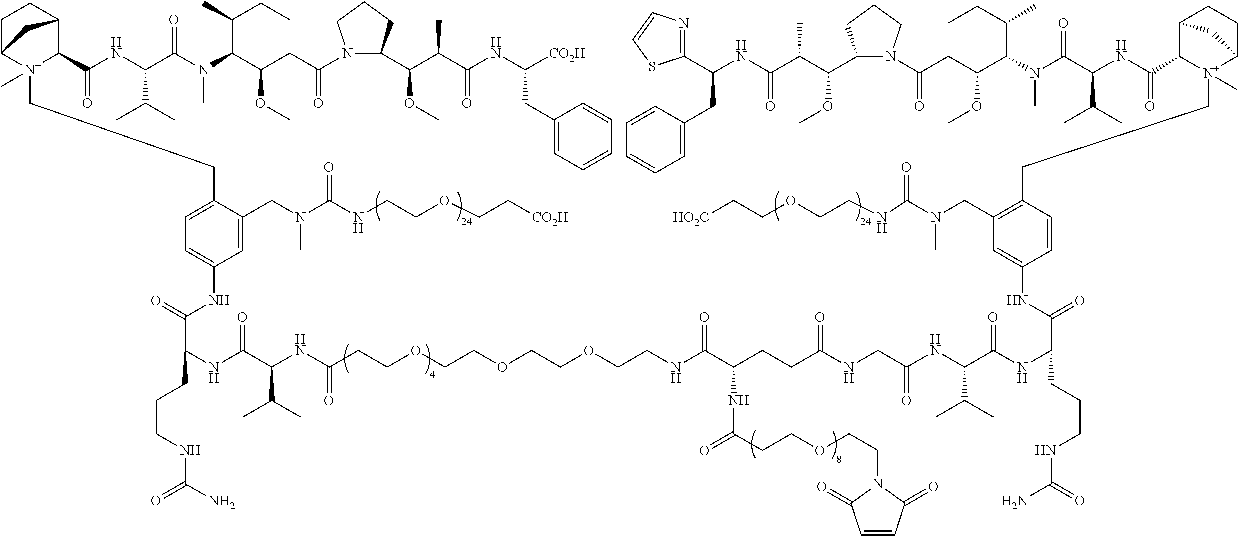 |
| P3- L13-P3 | 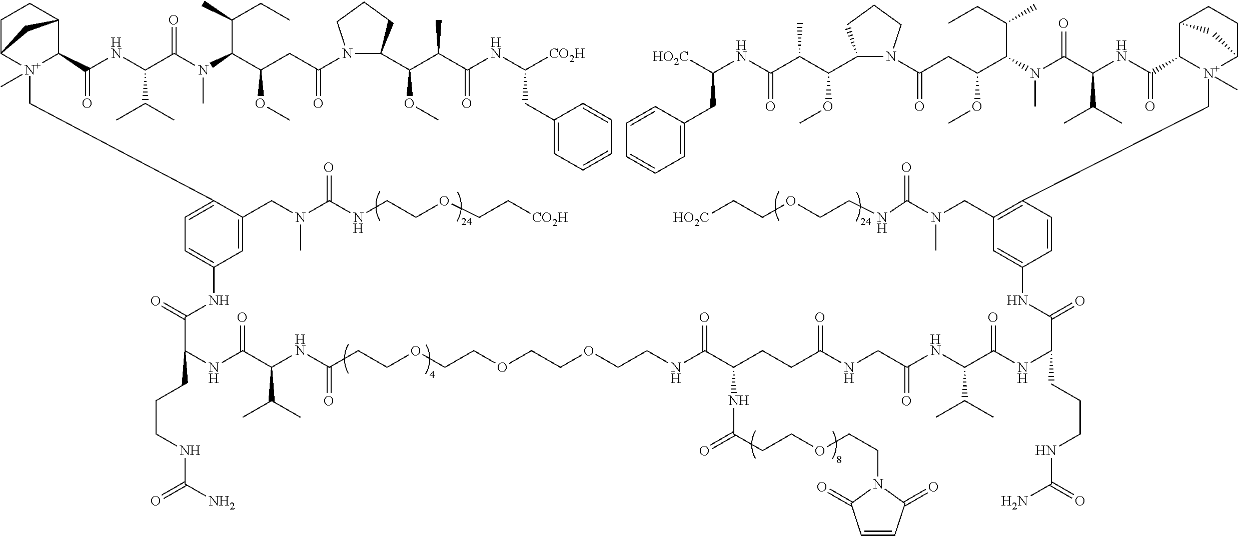 |
| P3- L13-P4 | 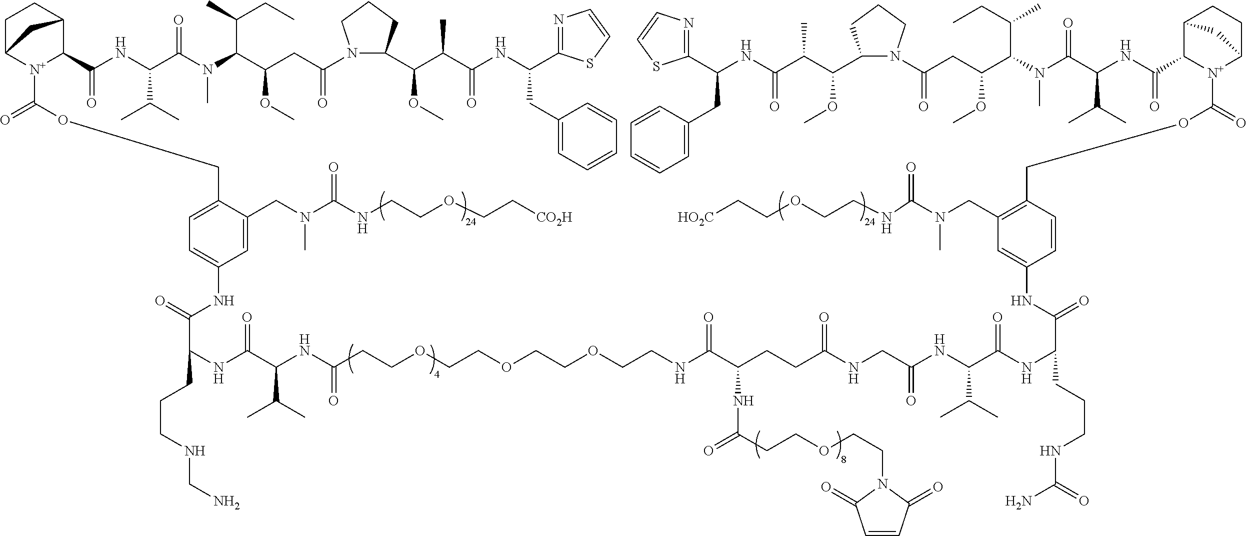 |
| P4- L13-P3 | 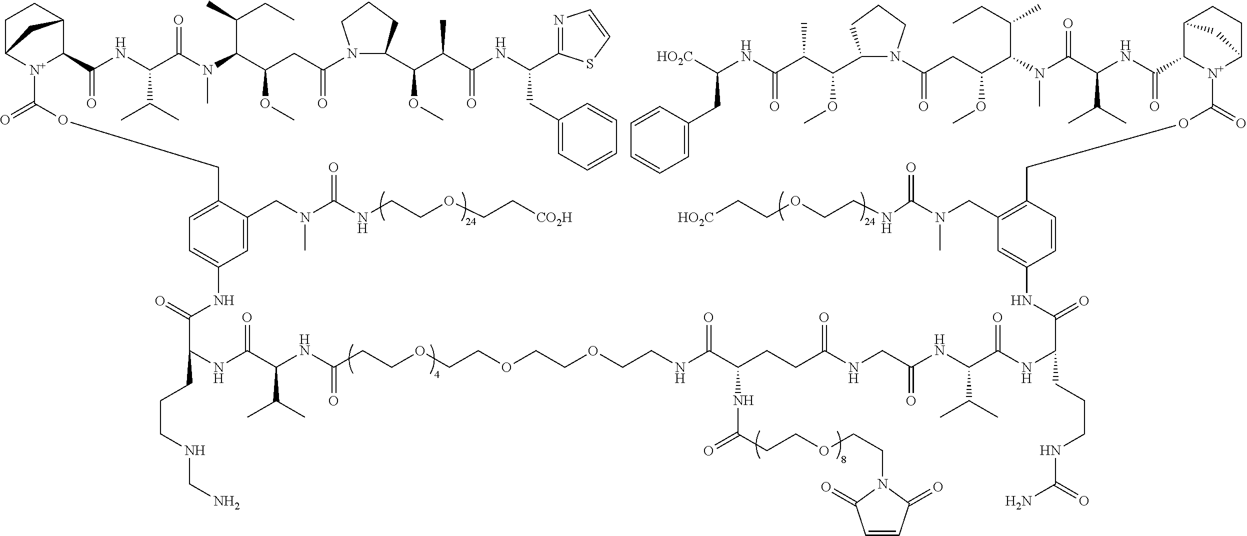 |
| P4- L13-P4 | 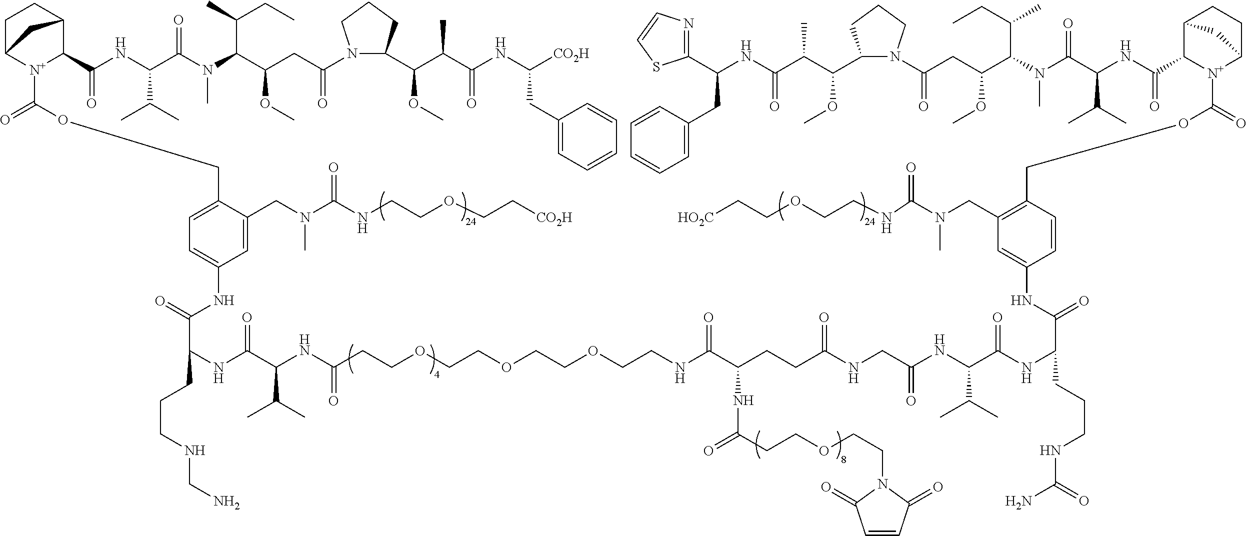 |
| P1- L14-P1 | 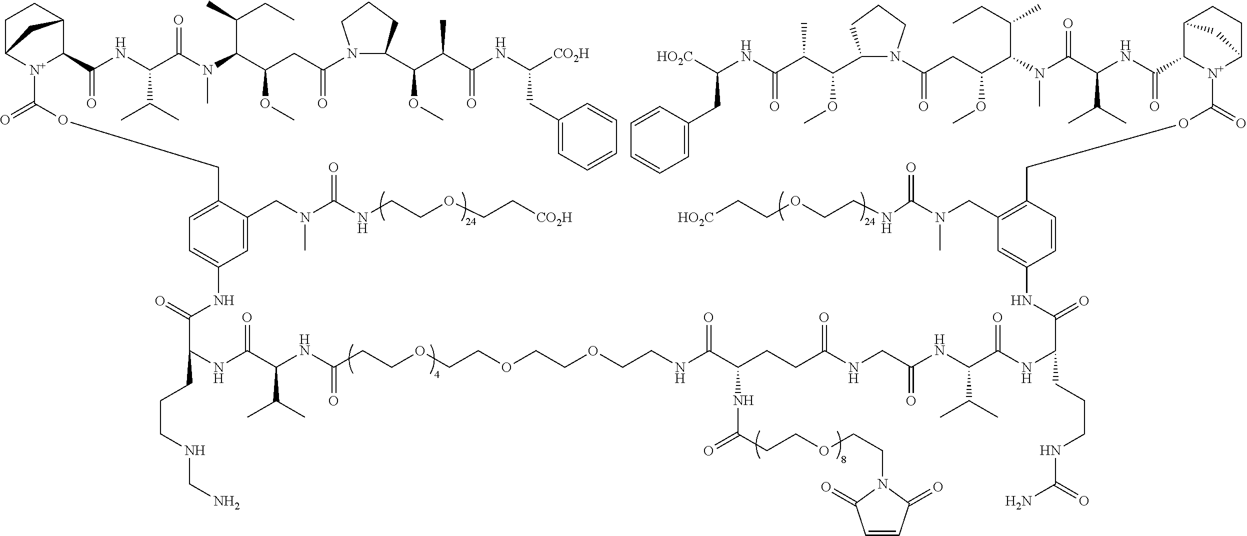 |
| P1- L14-P2 | 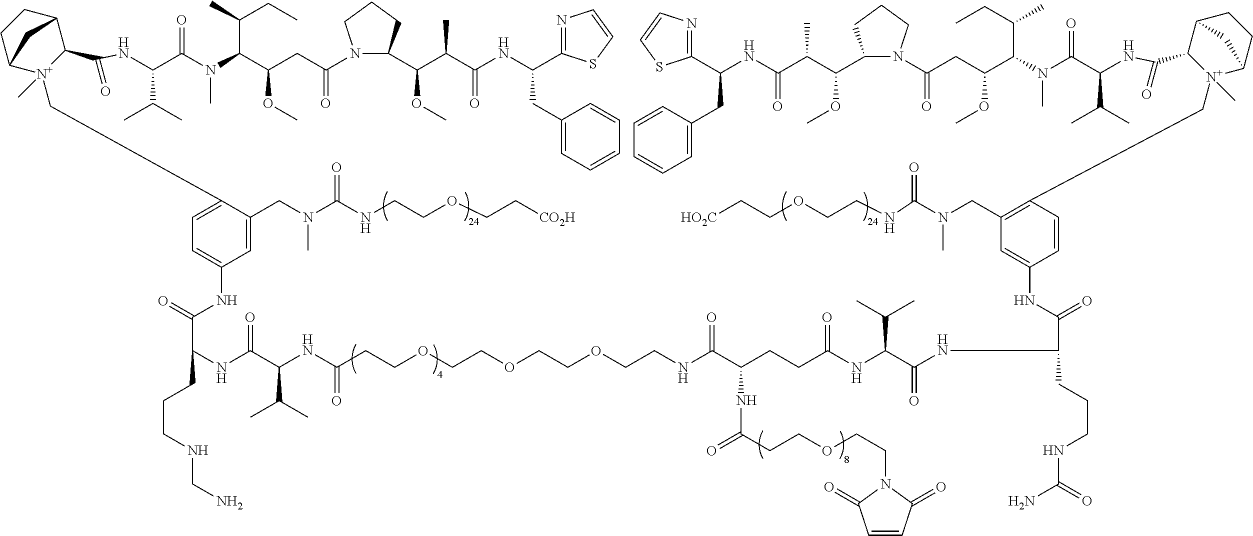 |
| P2- L14-P1 | 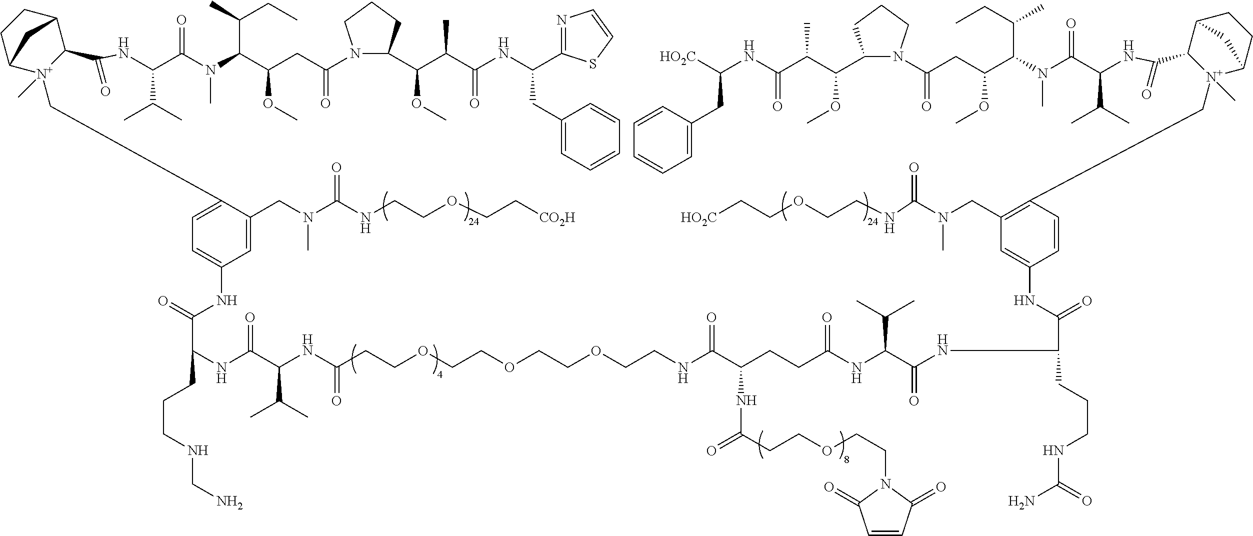 |
| P2- L14-P2 | 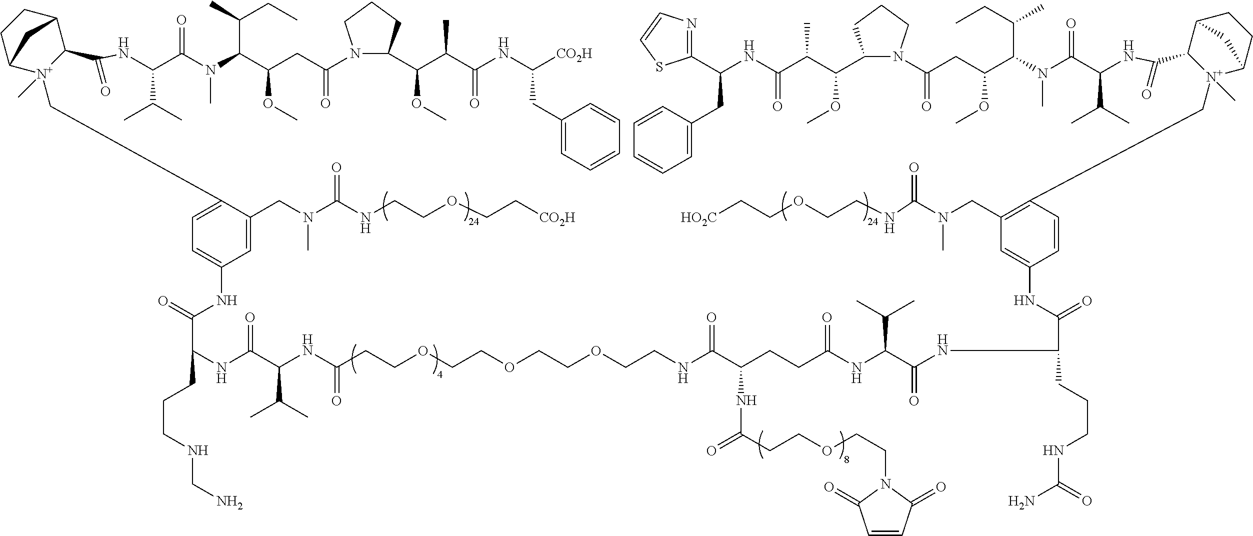 |
| P3- L14-P3 | 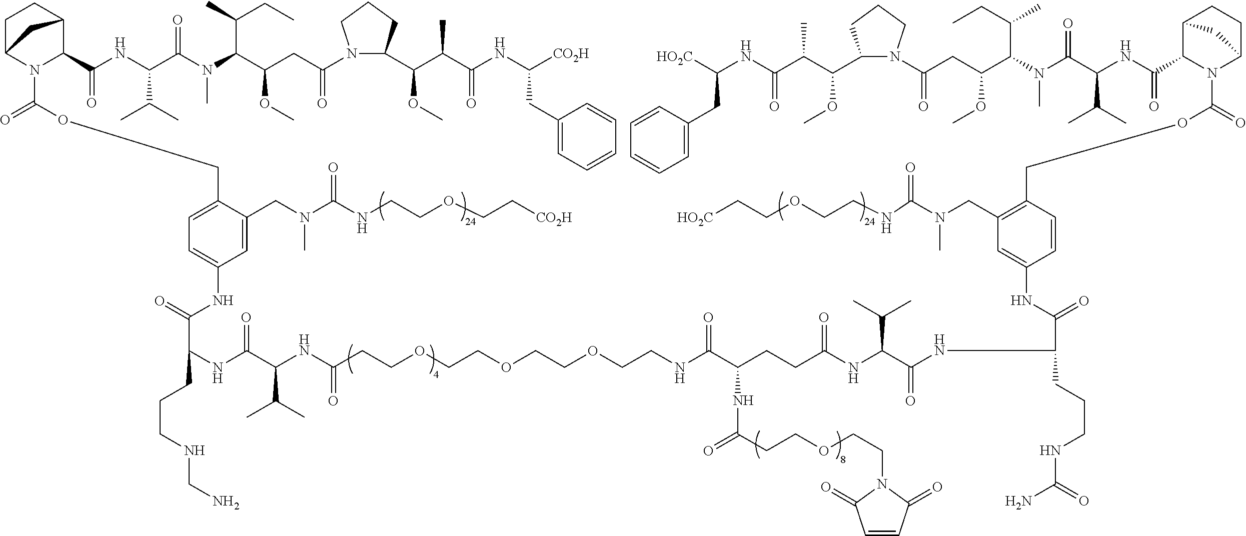 |
| P3- L14-P4 | 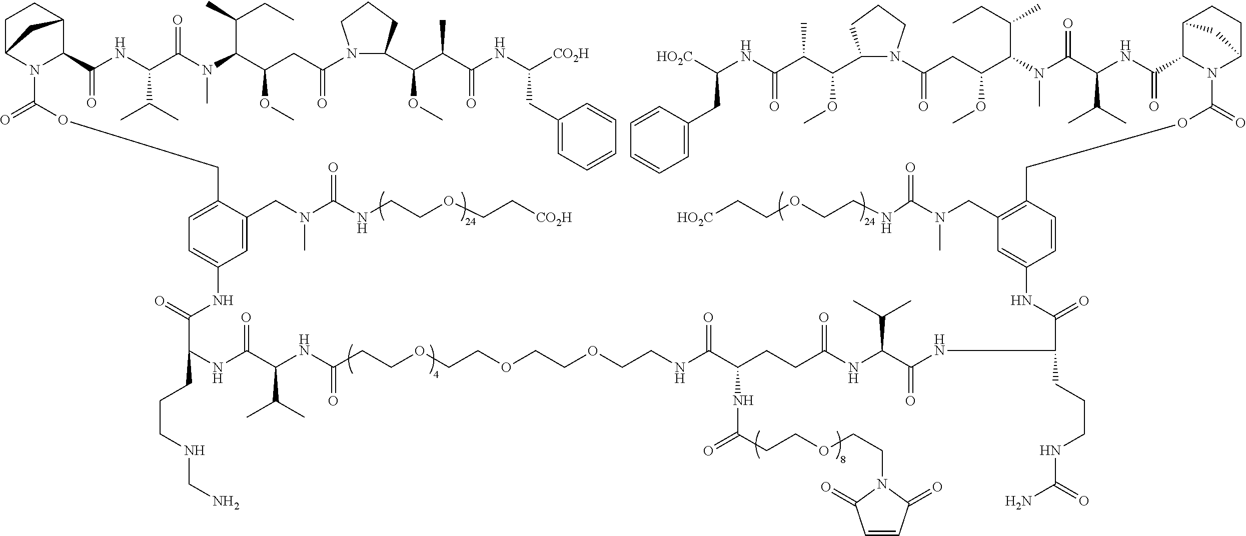 |
| P4- L14-P3 | 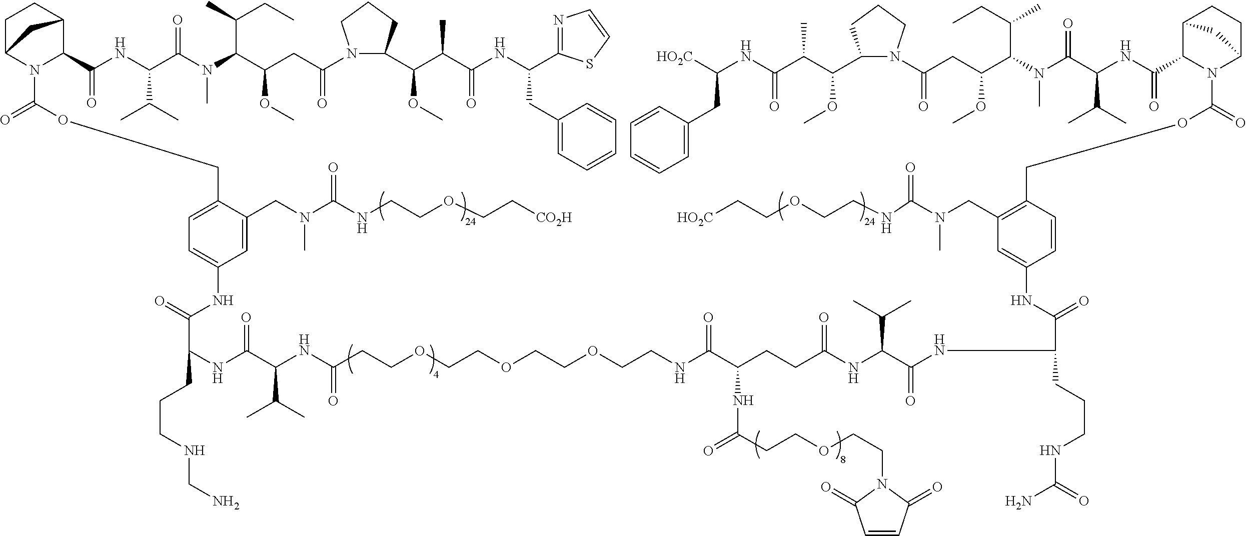 |
| P4- L14-P4 | 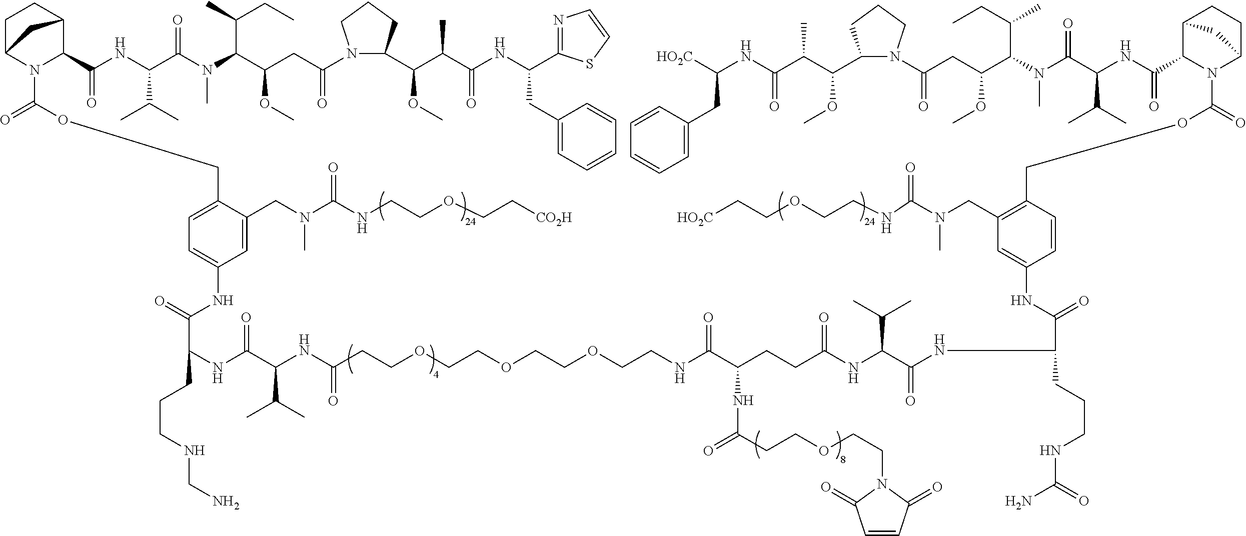 |
| P1- L15-P1 | 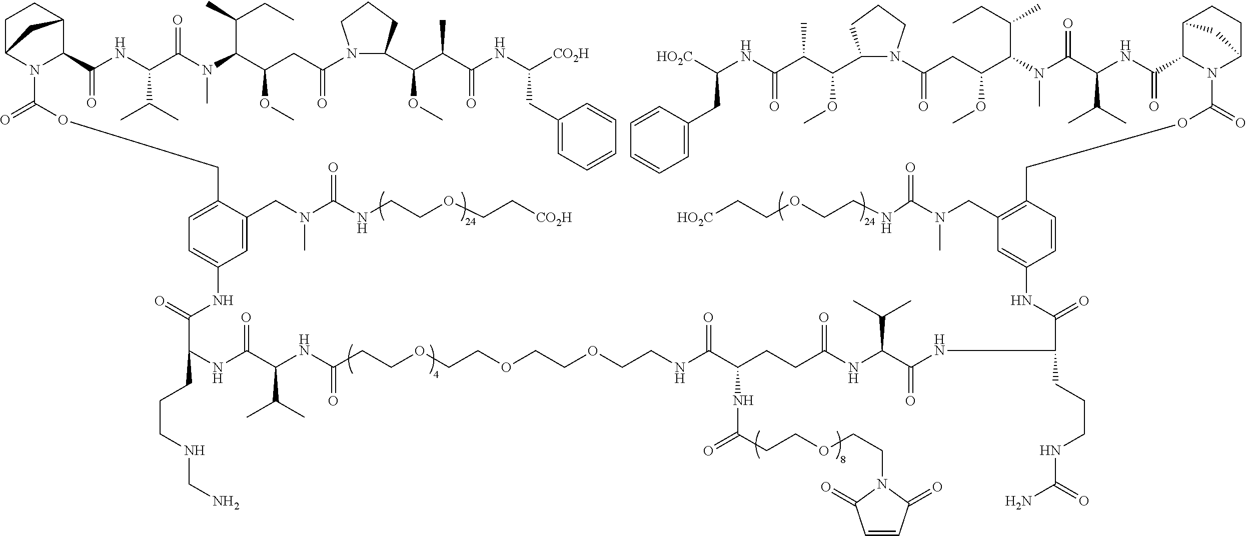 |
| P1- L15-P2 | 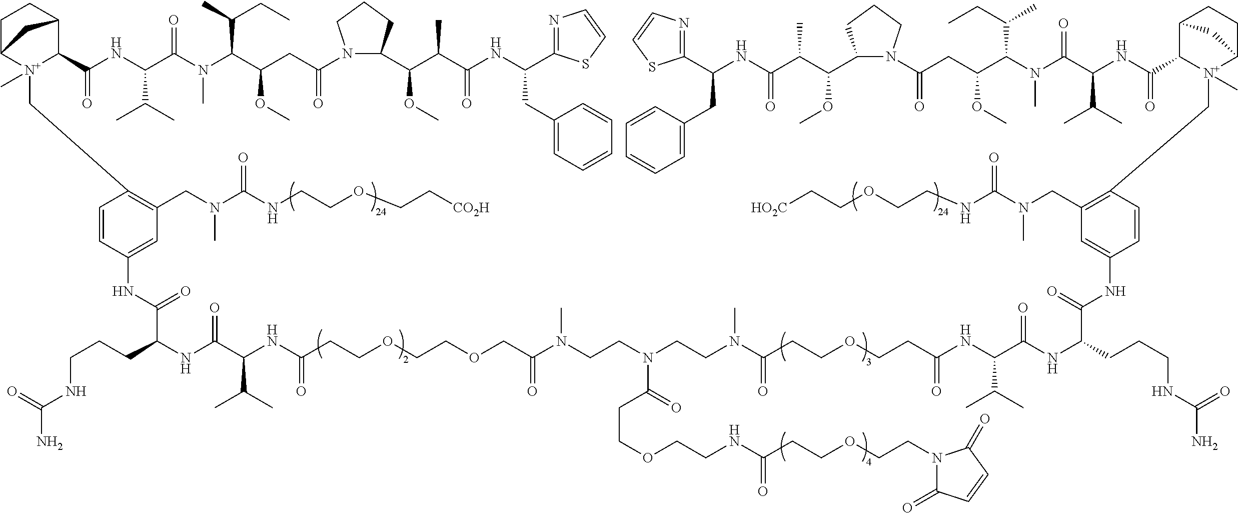 |
| P2- L15-P1 | 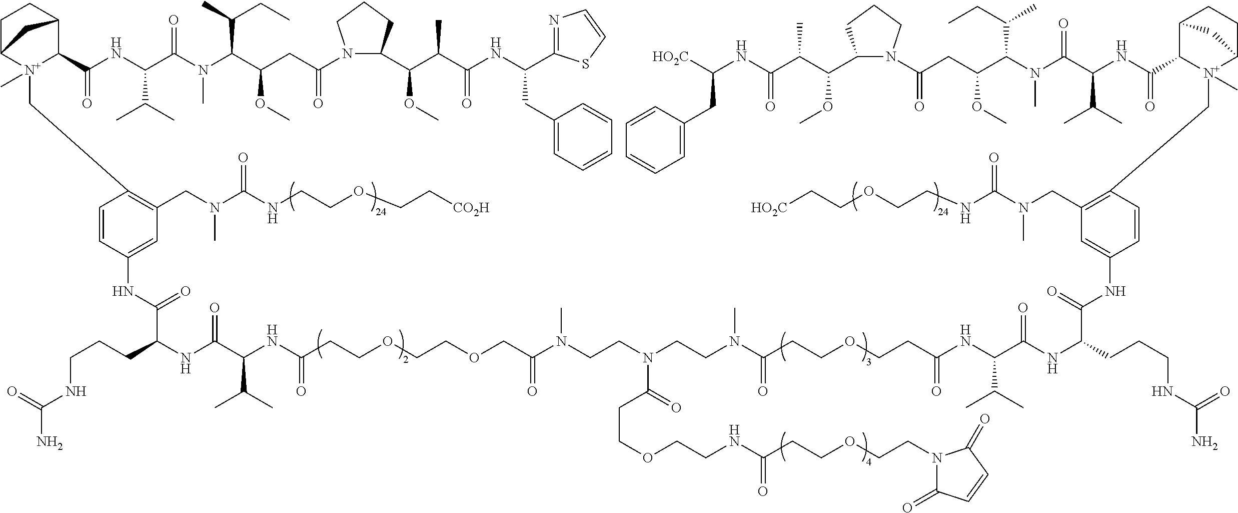 |
| P2- L15-P2 | 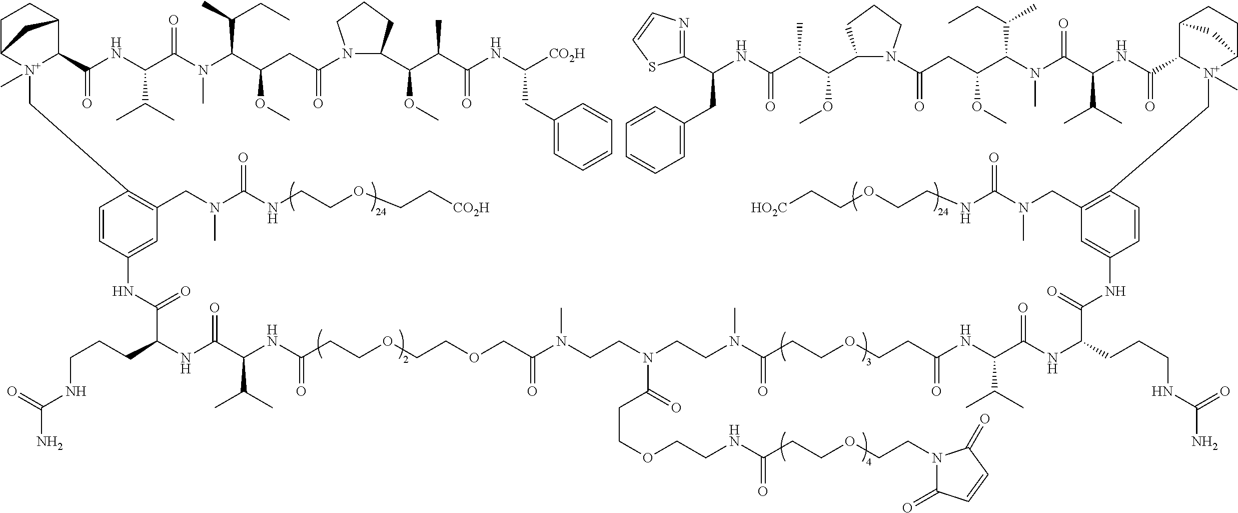 |
| P3- L15-P3 | 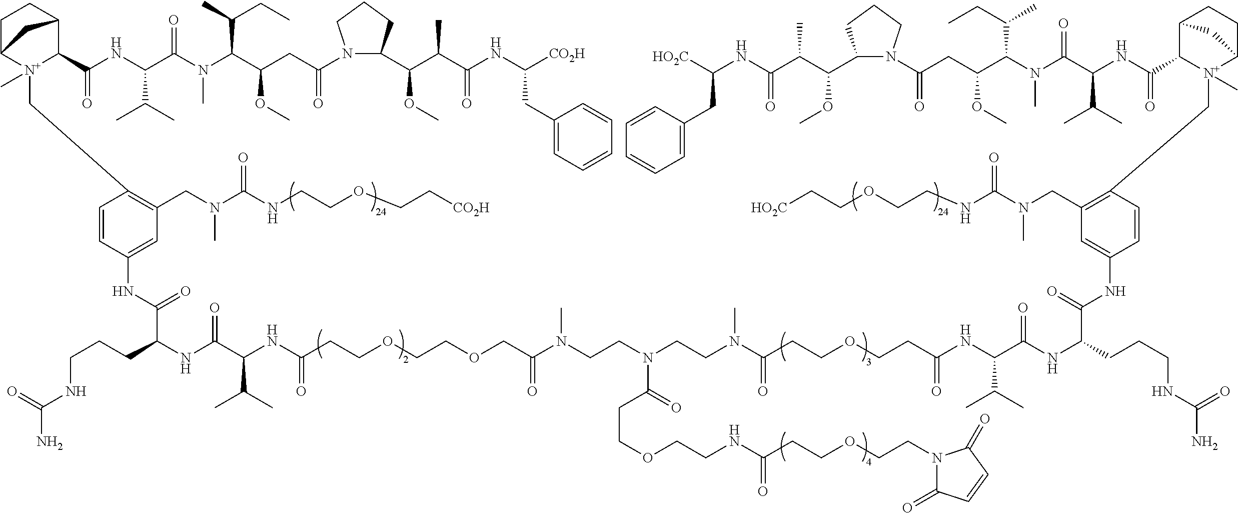 |
| P3- L15-P4 | 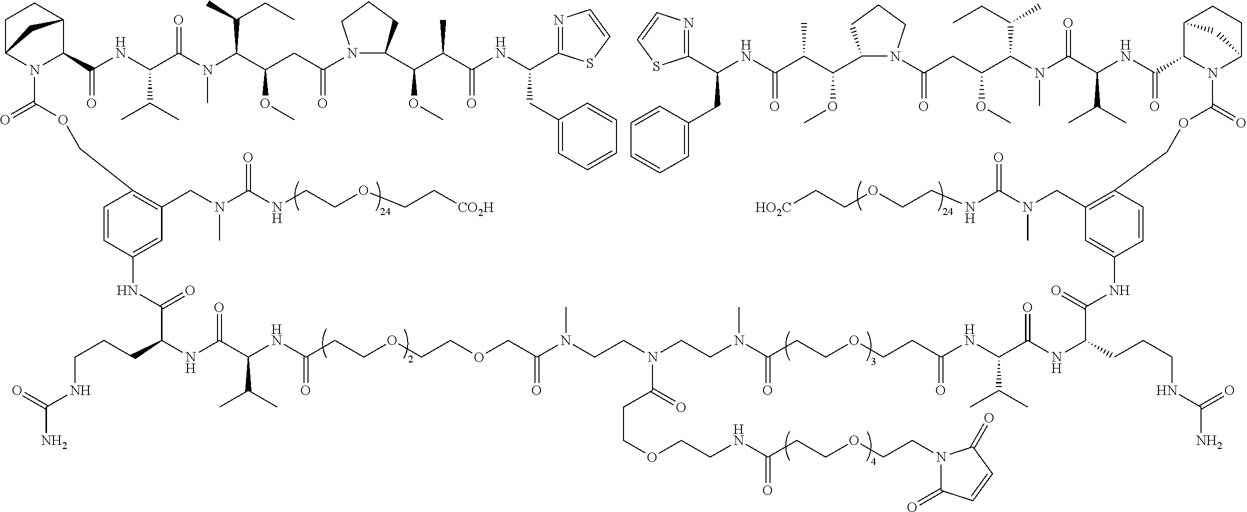 |
| P4- L15-P3 | 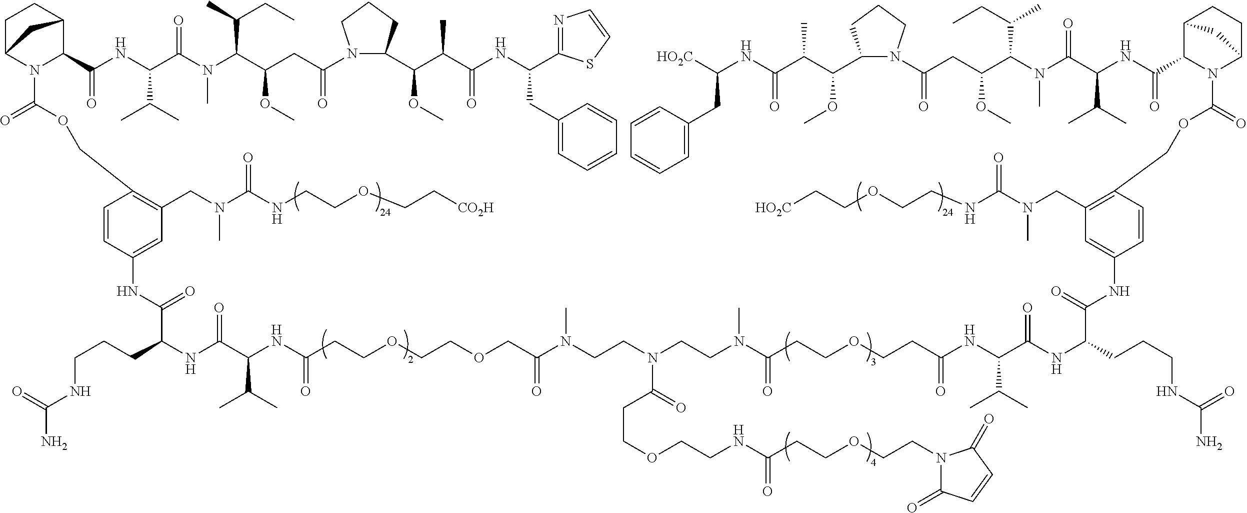 |
| P4- L15-P4 | 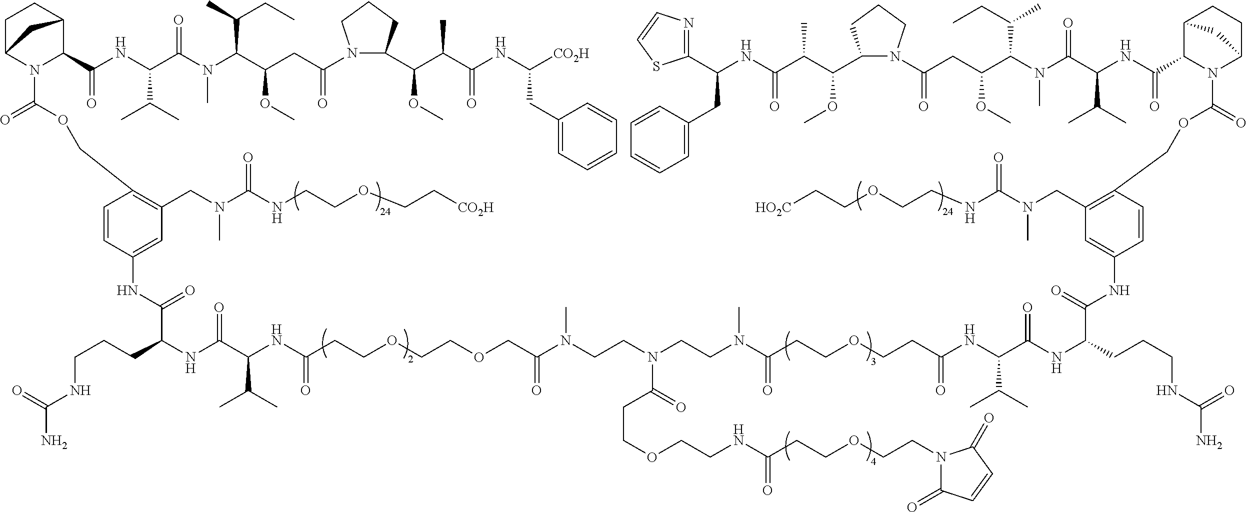 |
| P1- L16-P1 | 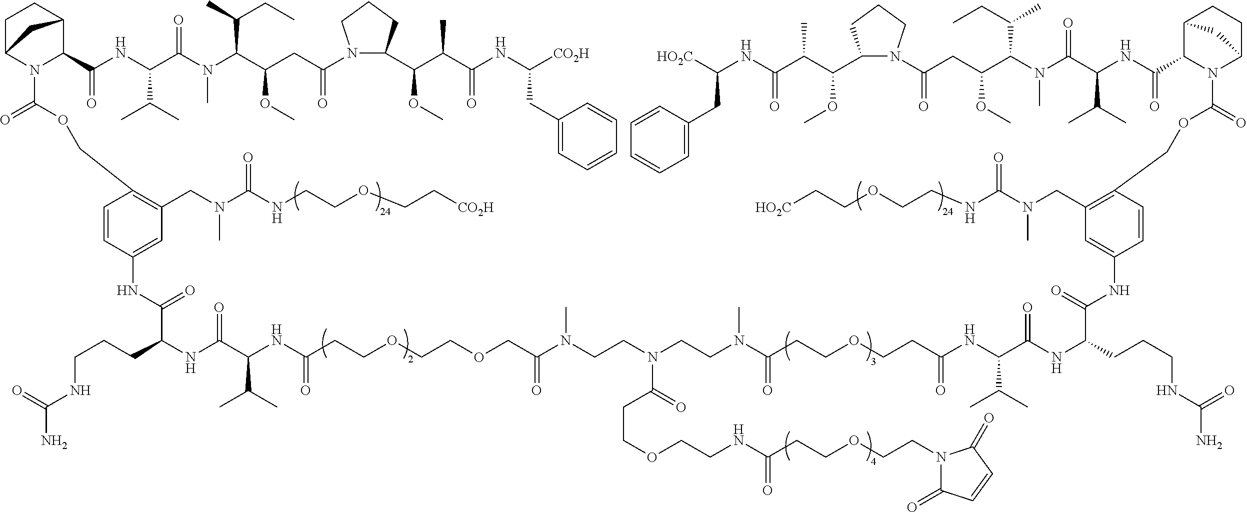 |
| P1- L16-P2 | 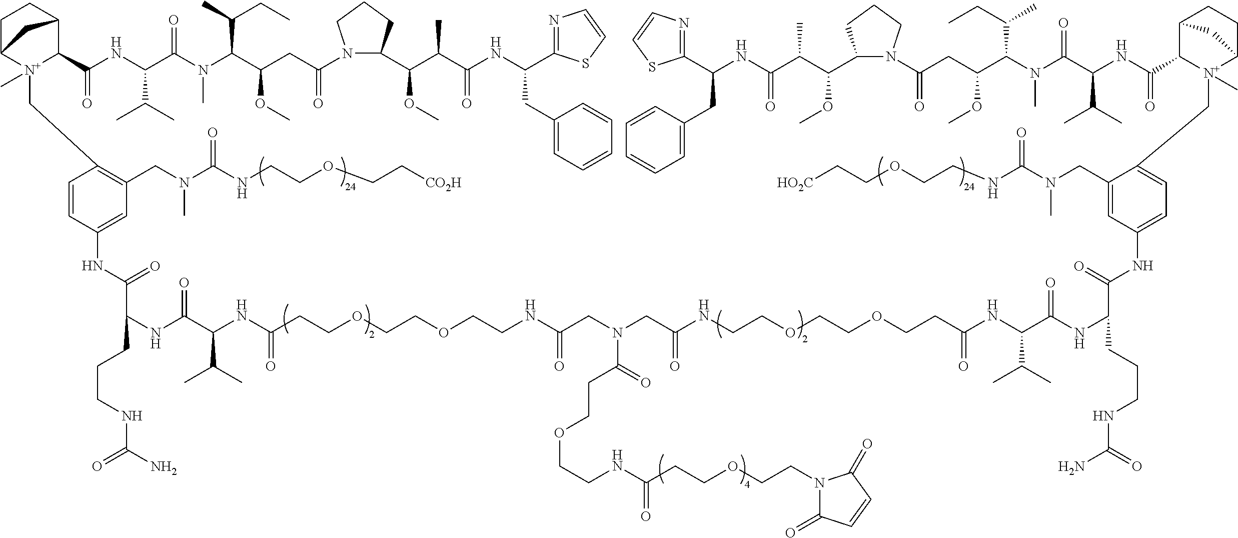 |
| P2- L16-P2 | 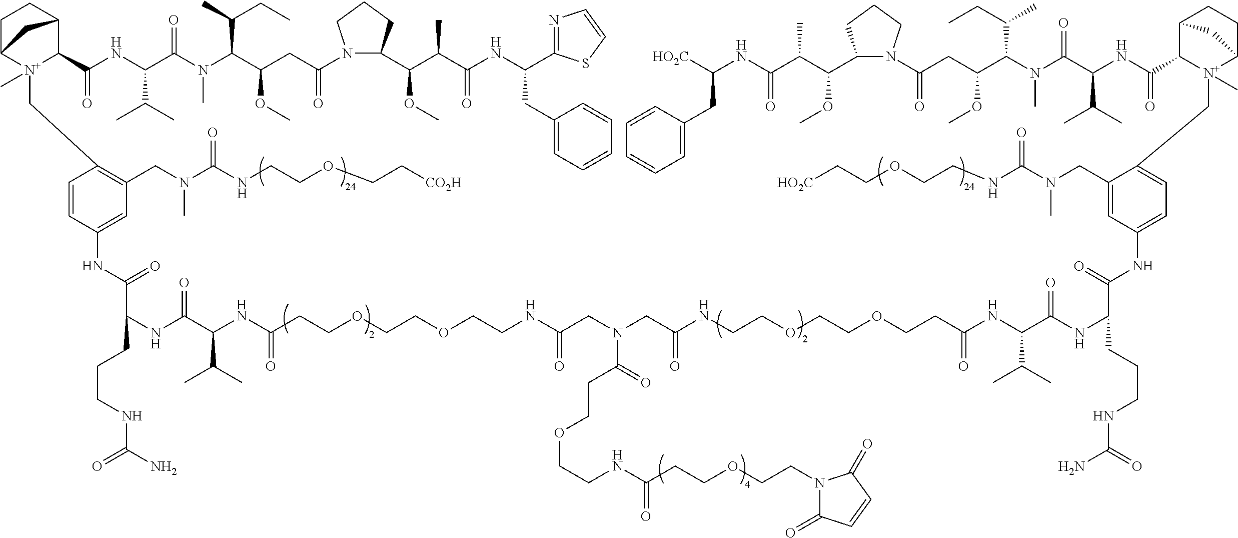 |
| P3- L16-P3 | 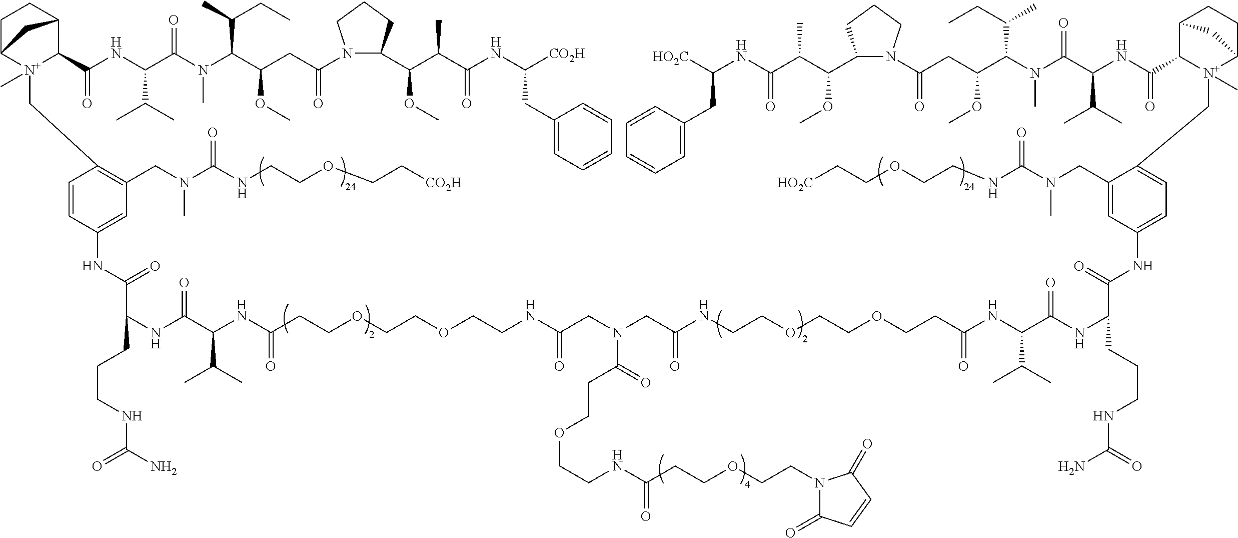 |
| P3- L16-P4 | 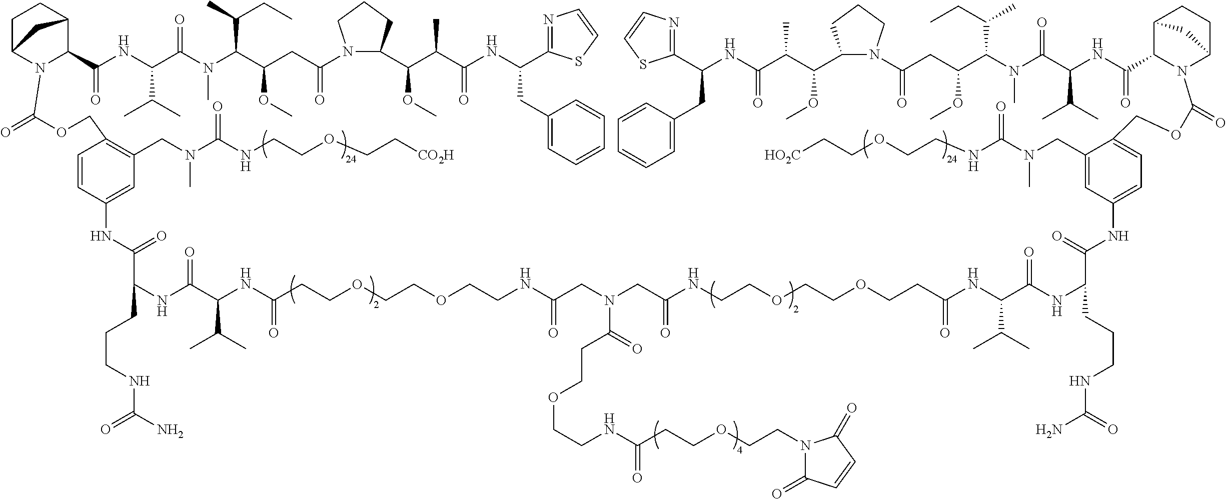 |
| P4- L16-P4 | 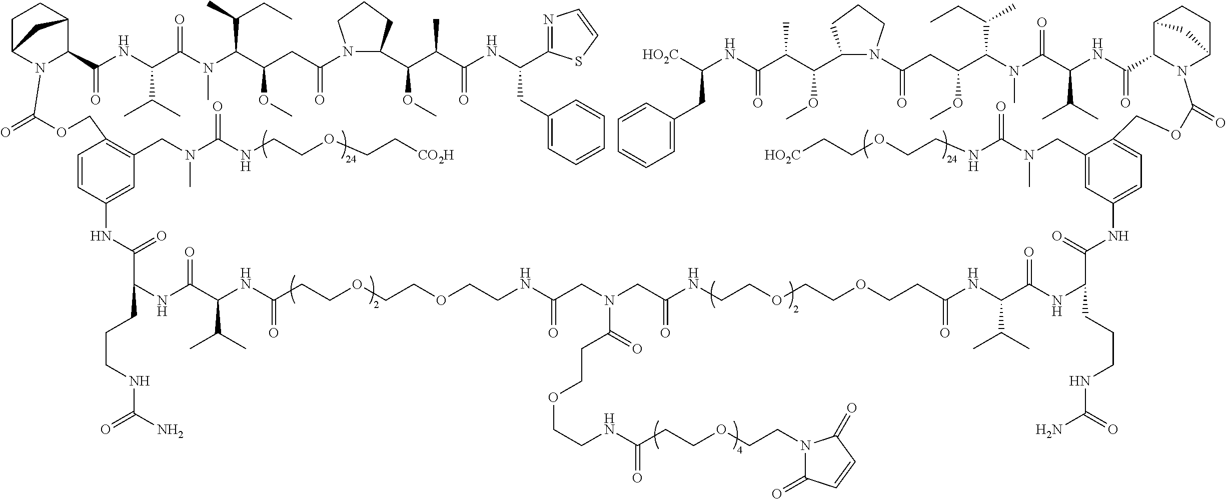 |
| P1- L17-P1 | 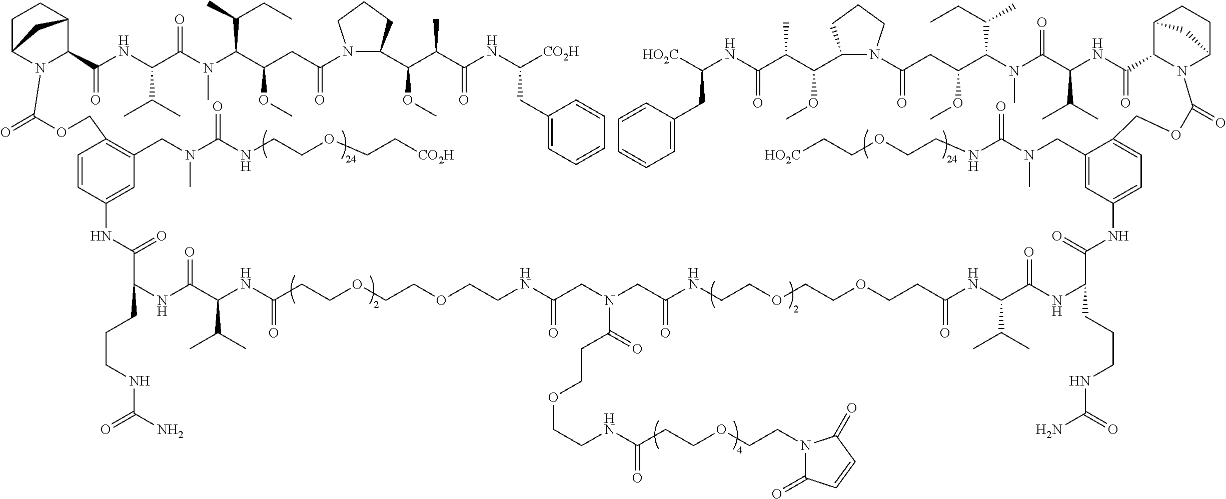 |
| P1- L17-P2 | 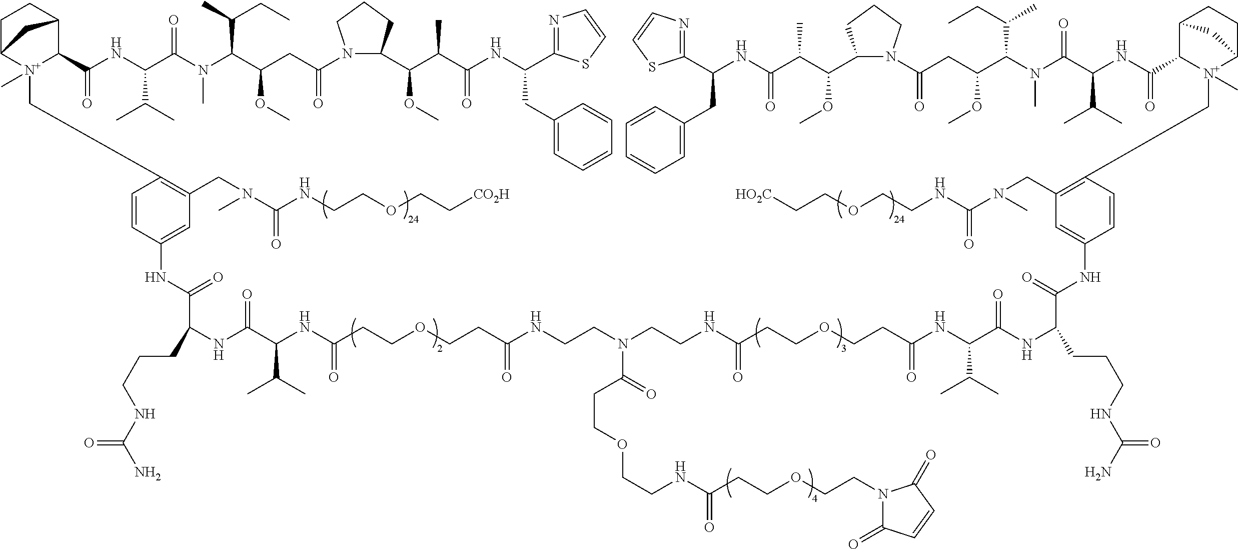 |
| P2- L17-P1 | 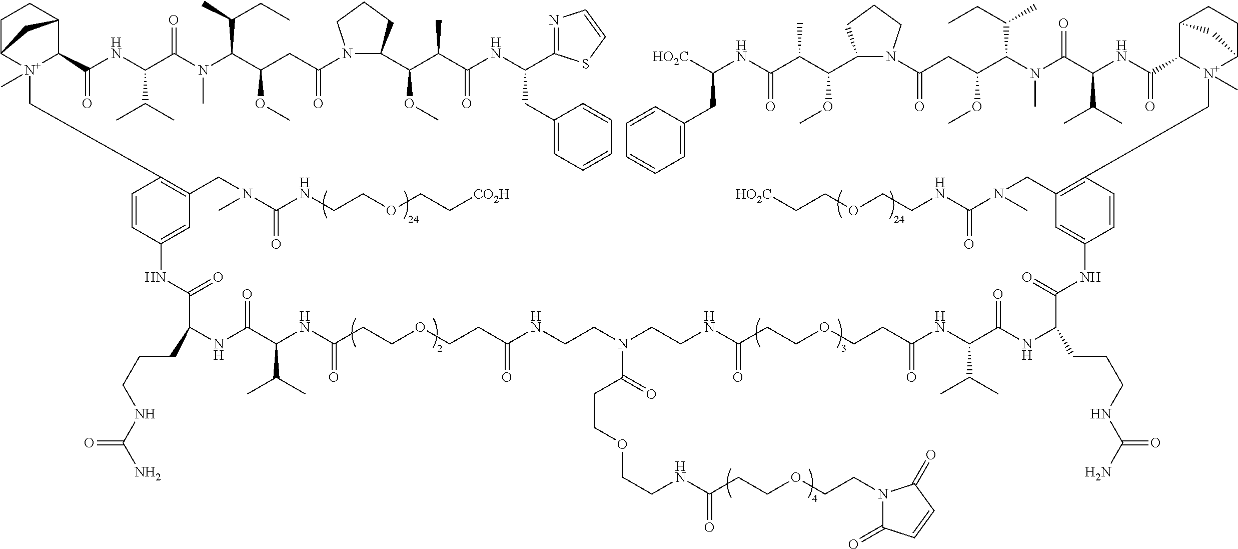 |
| P2- L17-P2 | 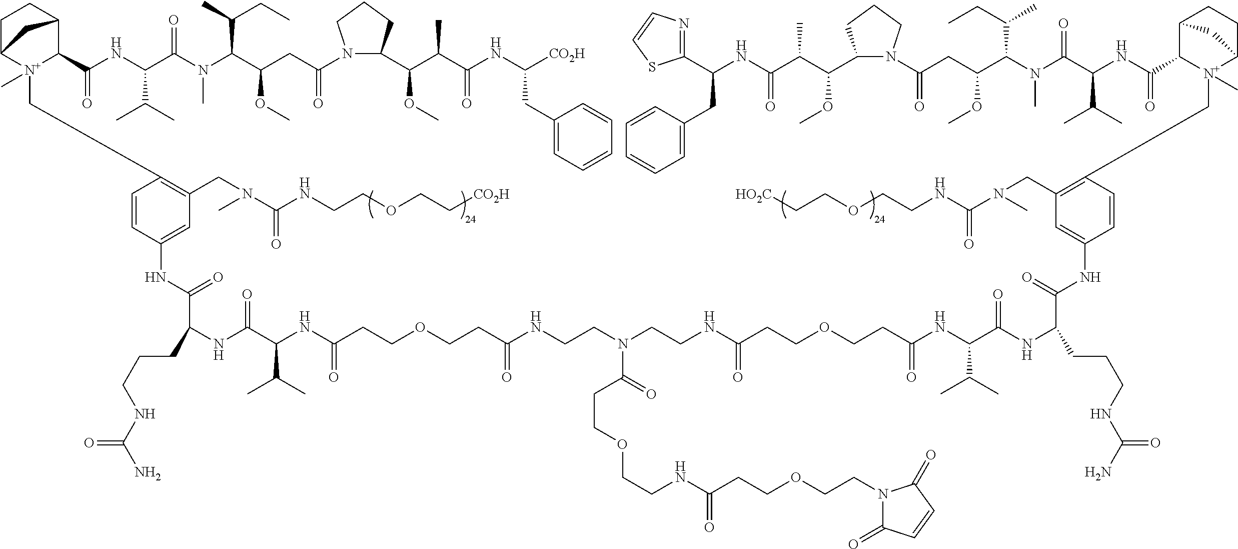 |
| P3- L17-P3 | 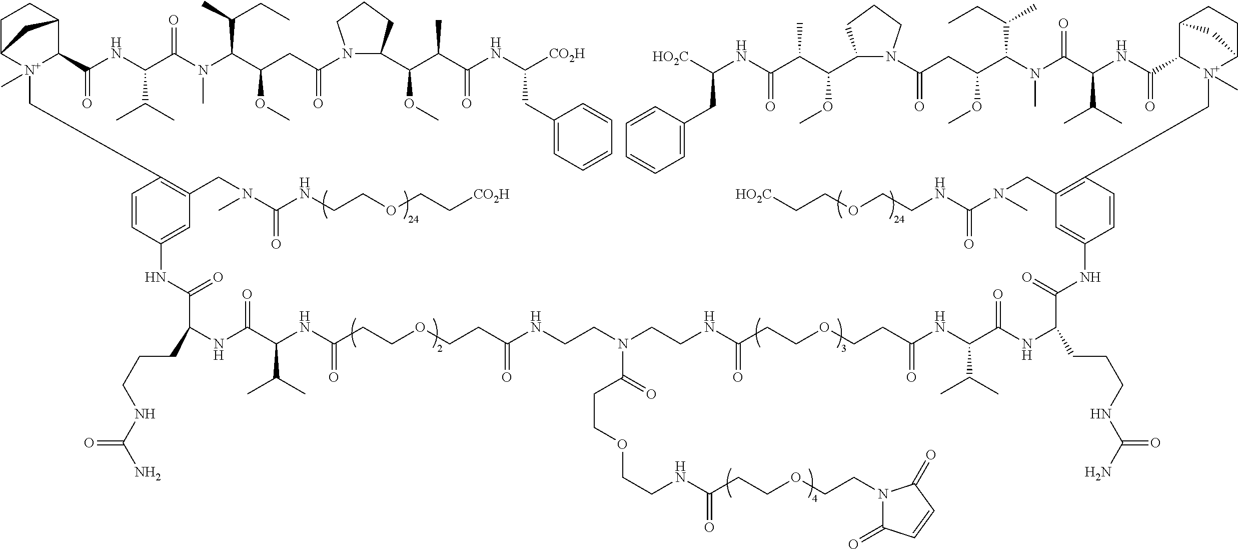 |
| P3- L17-P4 | 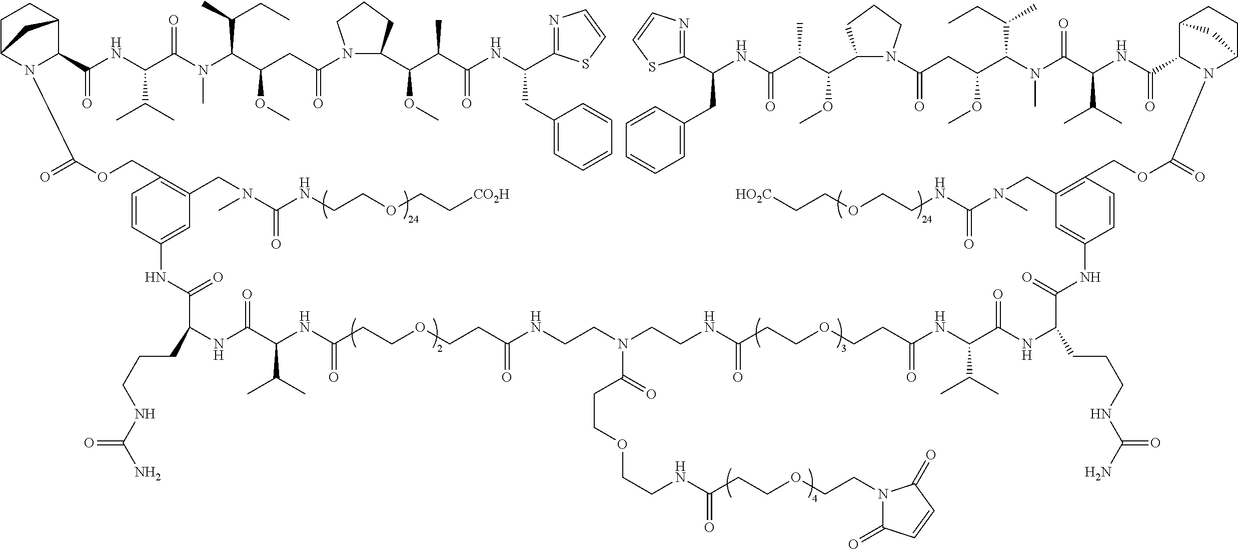 |
| P4- L17-P3 | 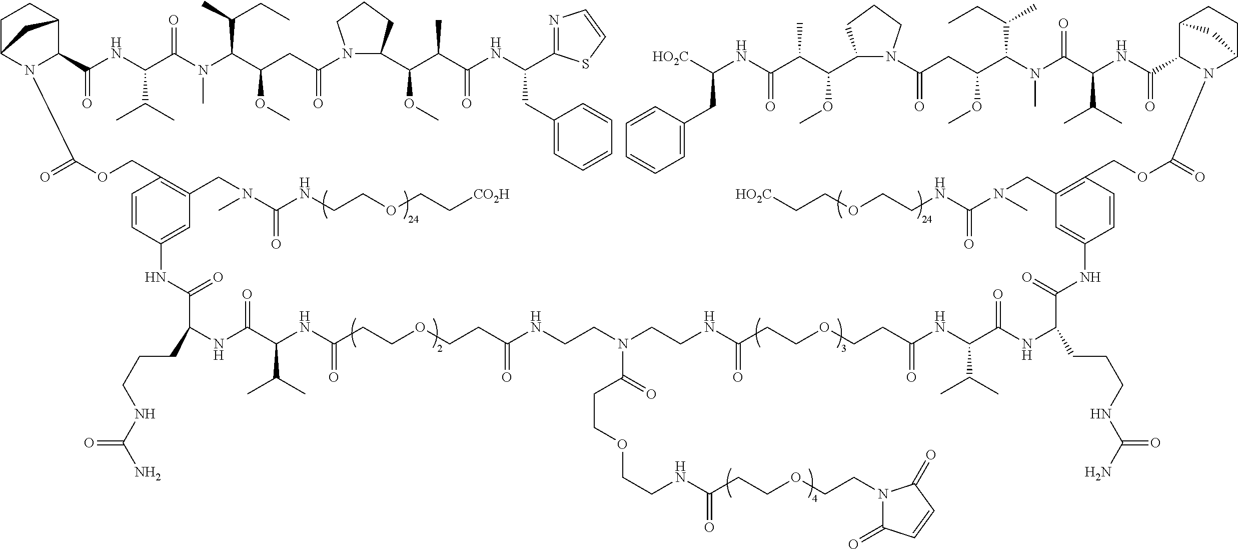 |
| P4- L17-P4 | 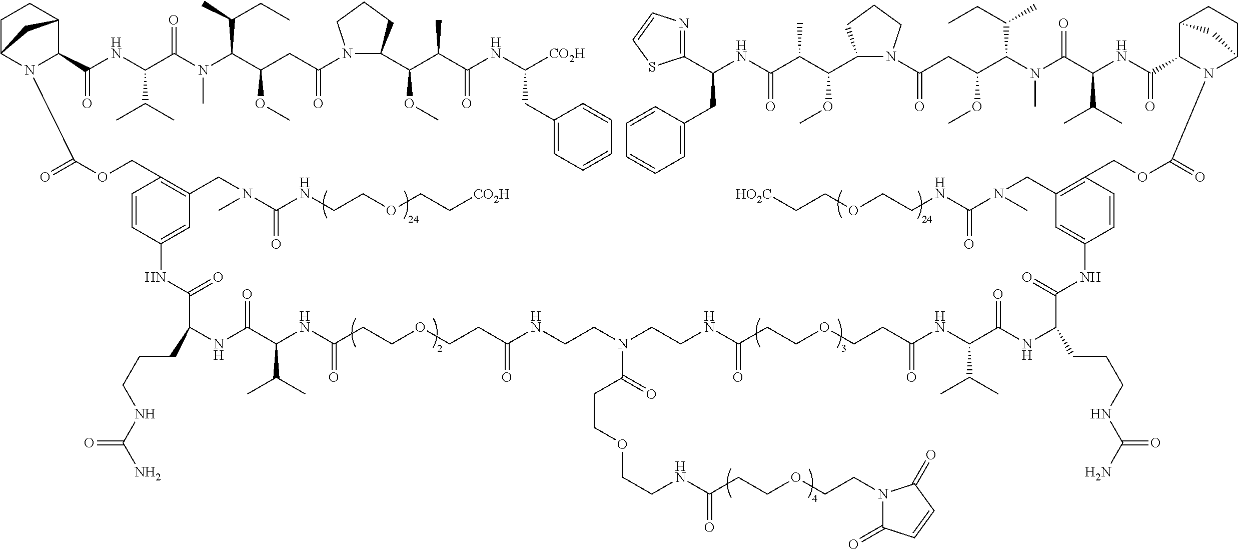 |
| P1- L18-P1 | 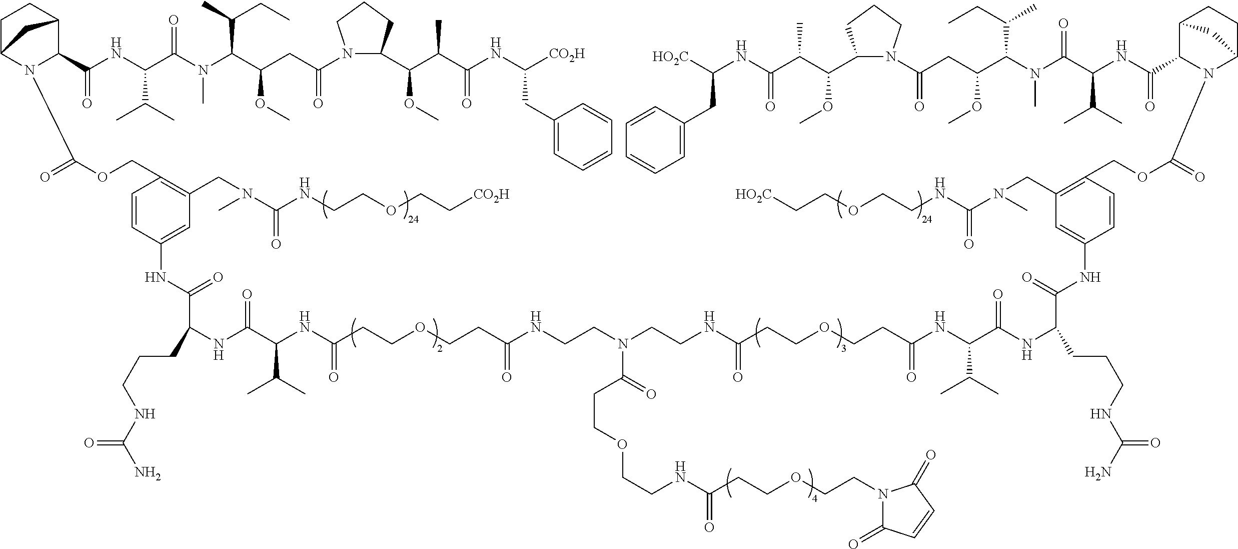 |
| P1- L18-P2 | 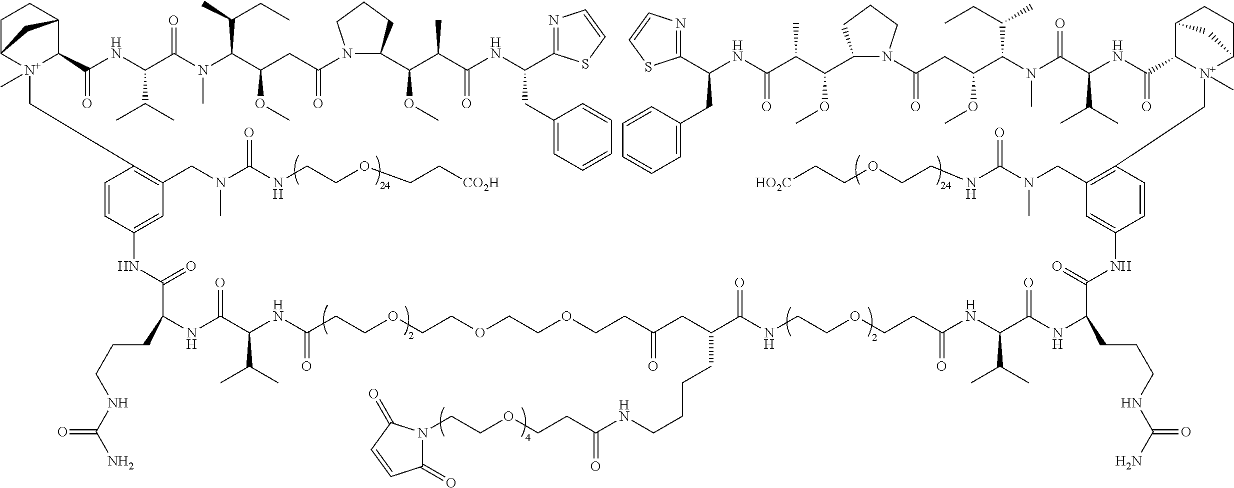 |
| P2- L18-P1 | 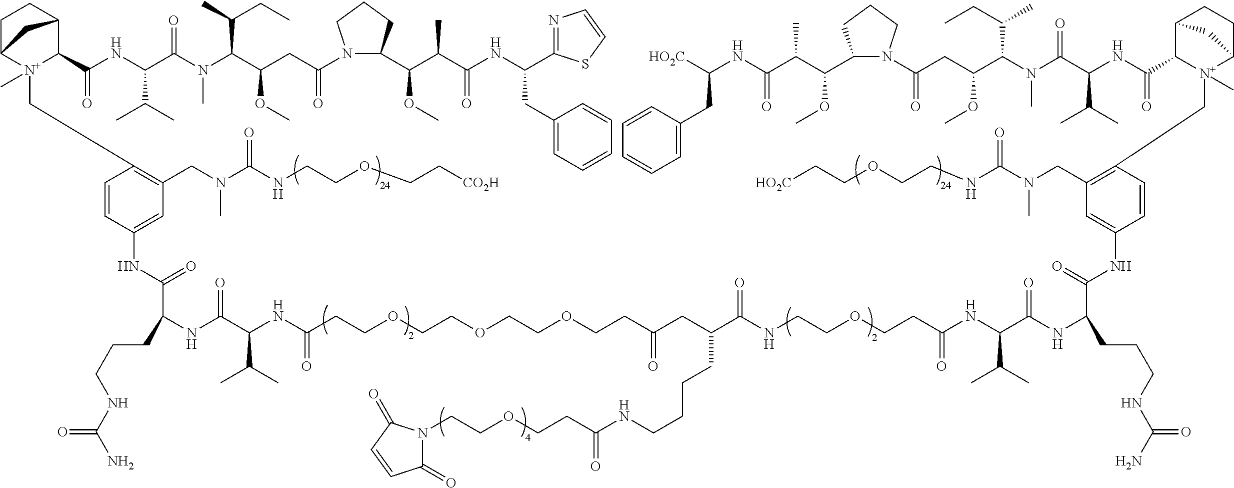 |
| P2- L18-P2 | 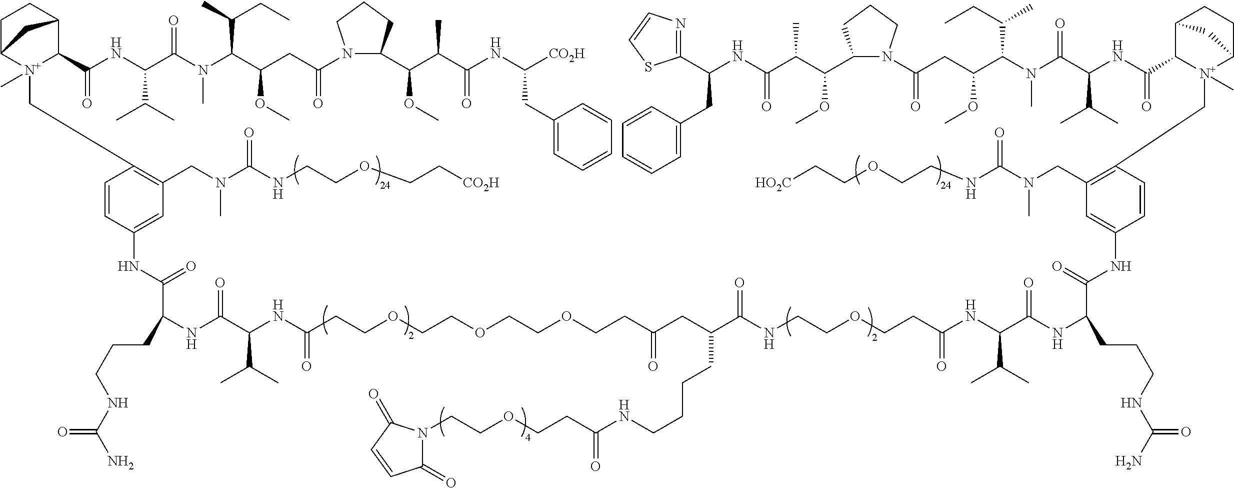 |
| P3- L18-P3 | 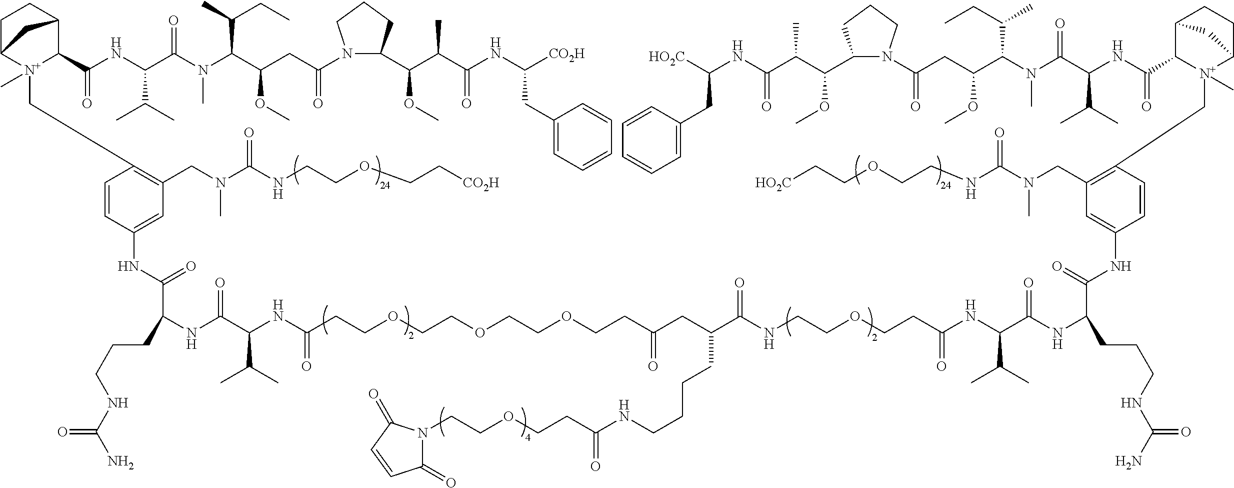 |
| P3- L18-P4 | 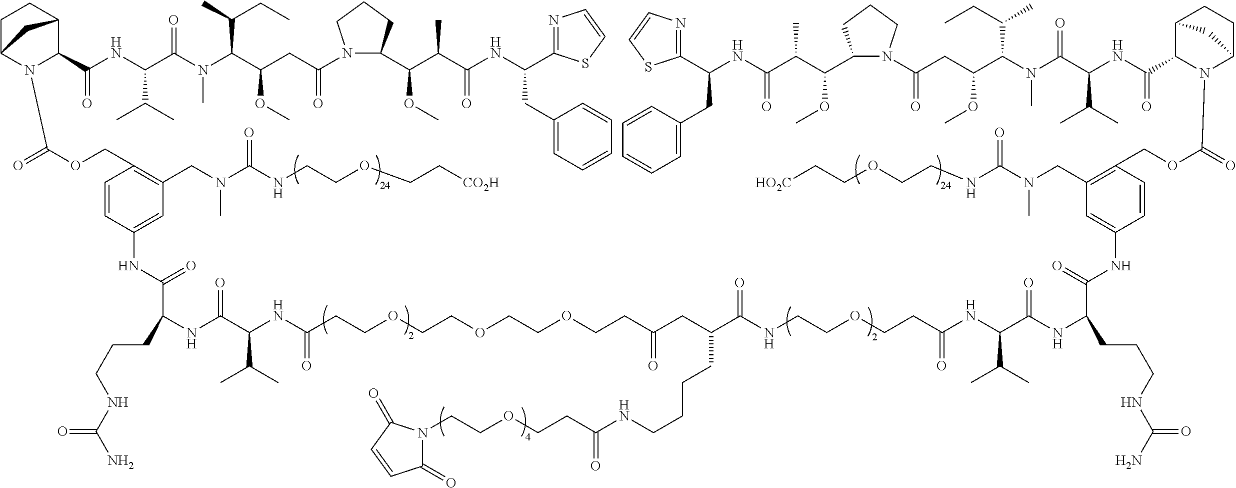 |
| P4- L18-P3 | 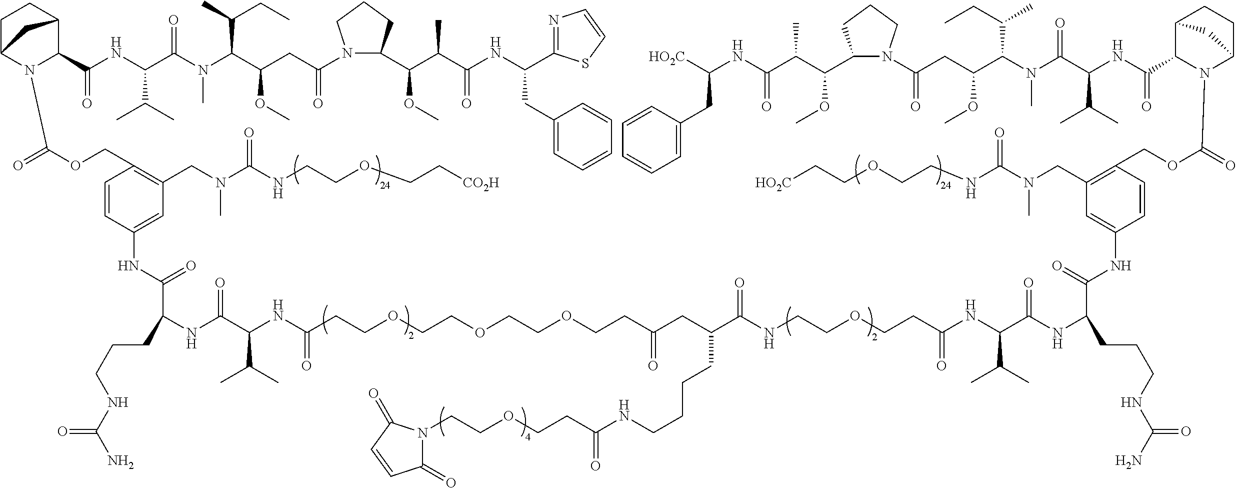 |
| P4- L18-P4 | 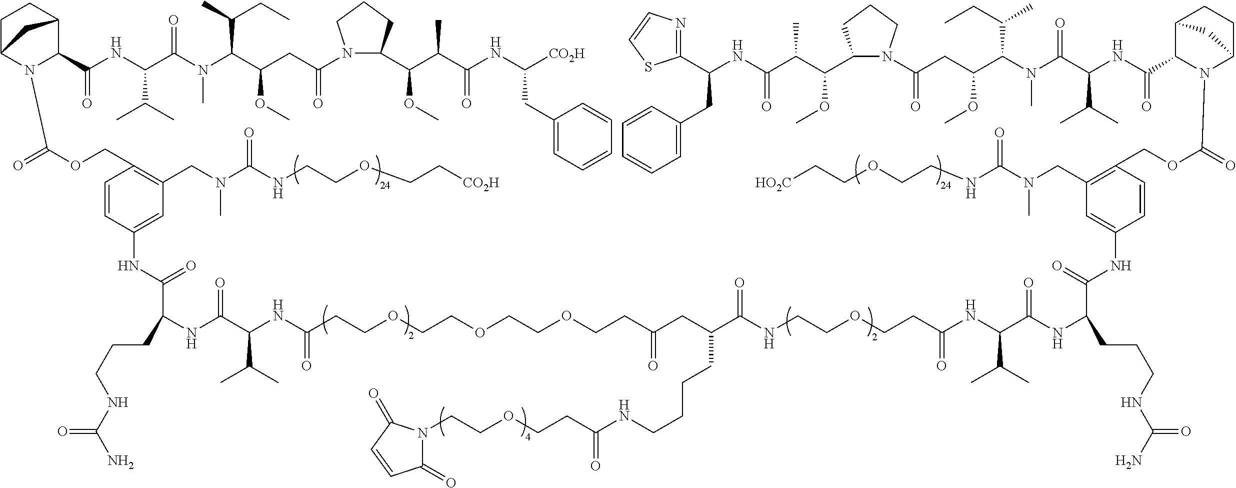 |
| P1- L19-P1 | 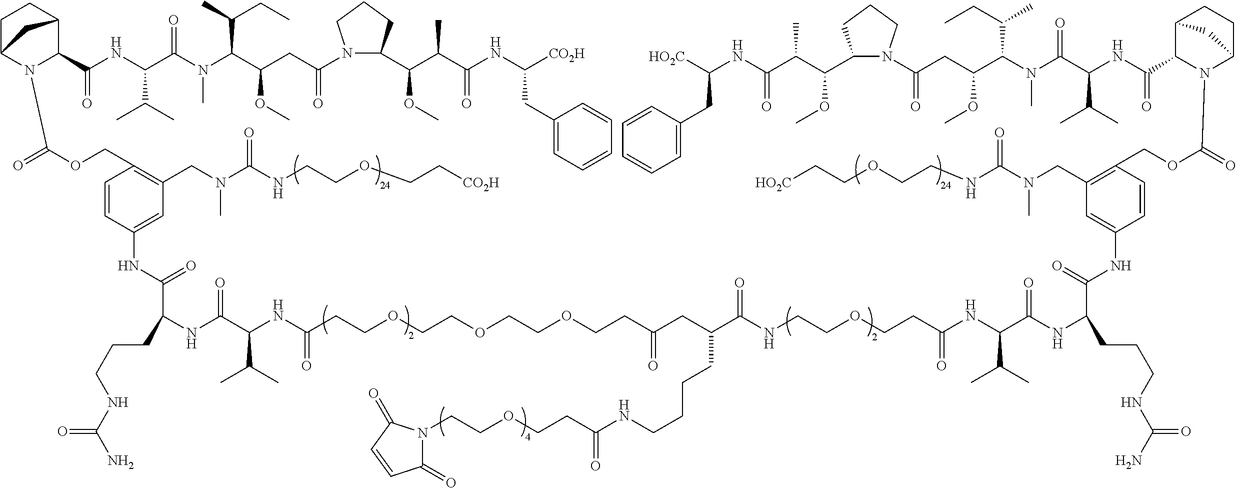 |
| P1- L19-P2 | 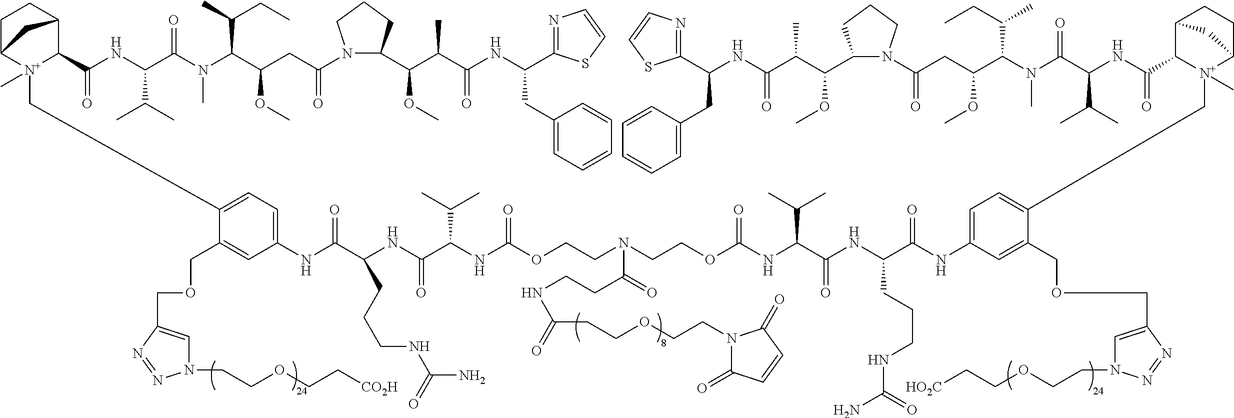 |
| P1- L19-P3 | 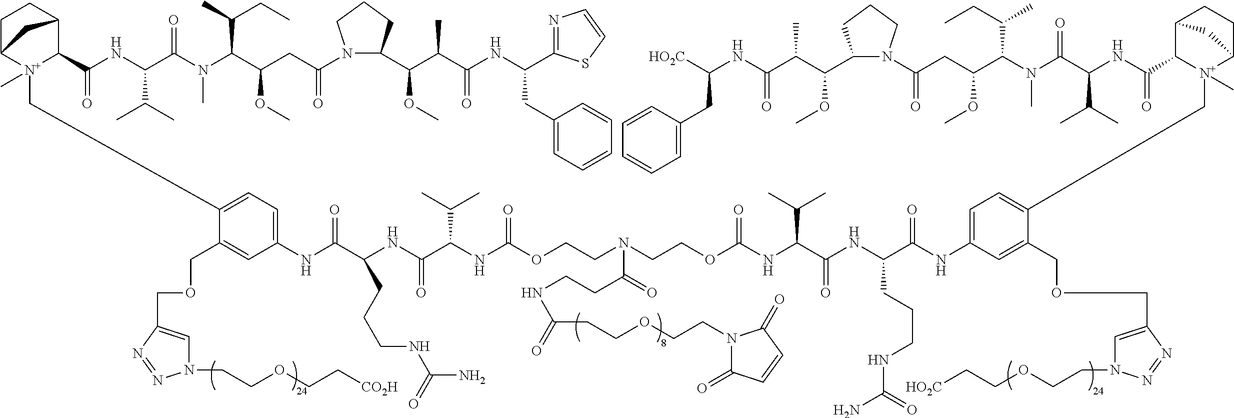 |
| P1- L19-P4 | 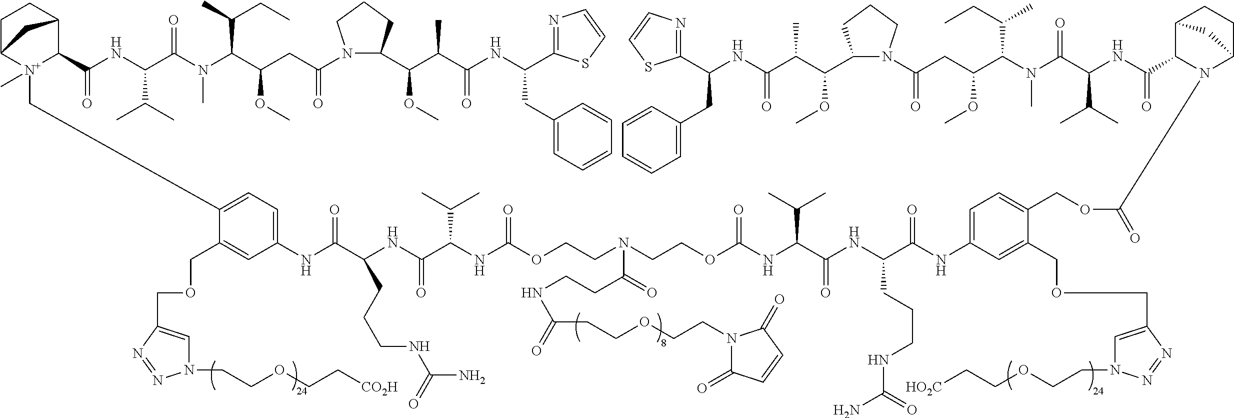 |
| P2- L19-P2 | 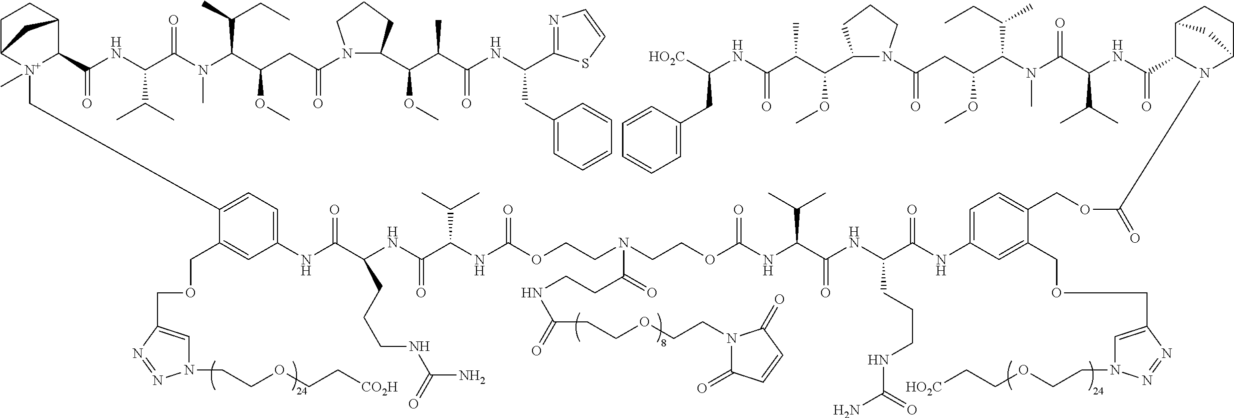 |
| P2- L19-P3 | 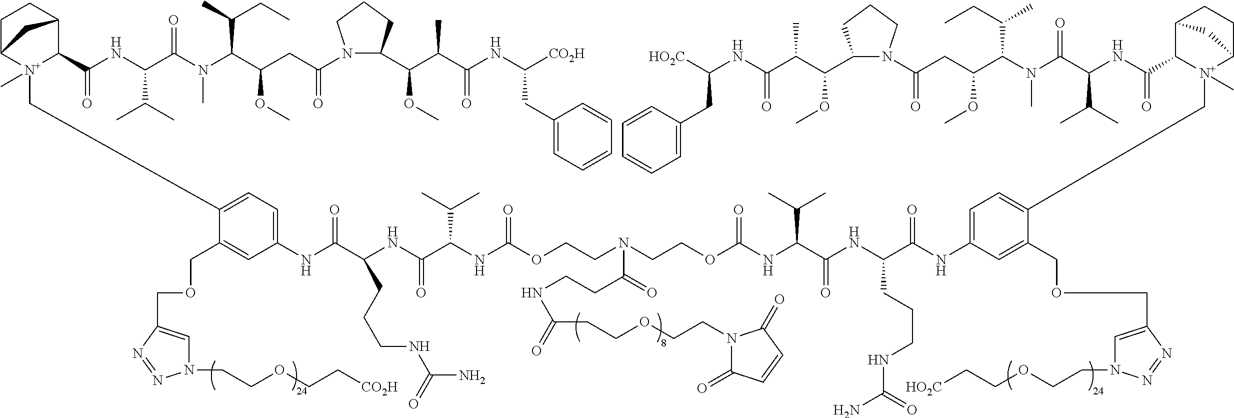 |
| P2- L19-P4 | 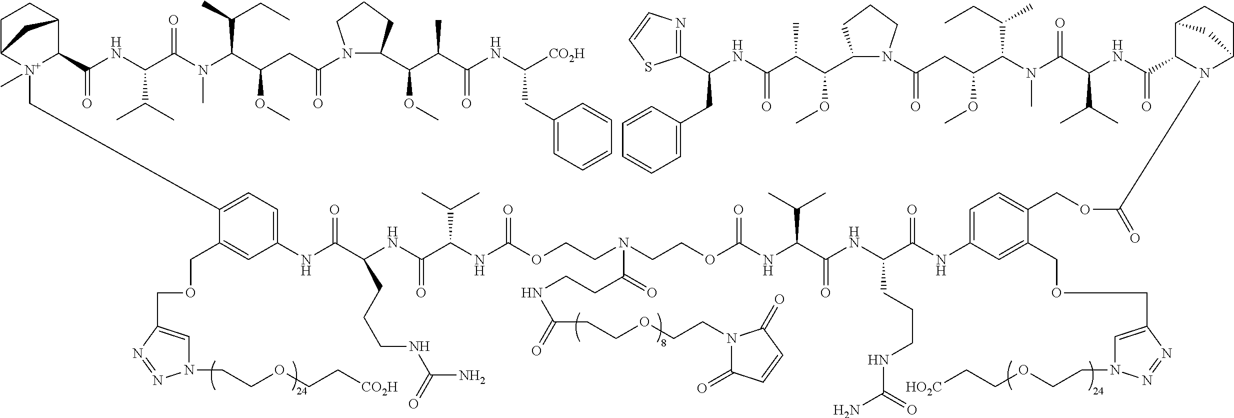 |
| P3- L19-P3 | 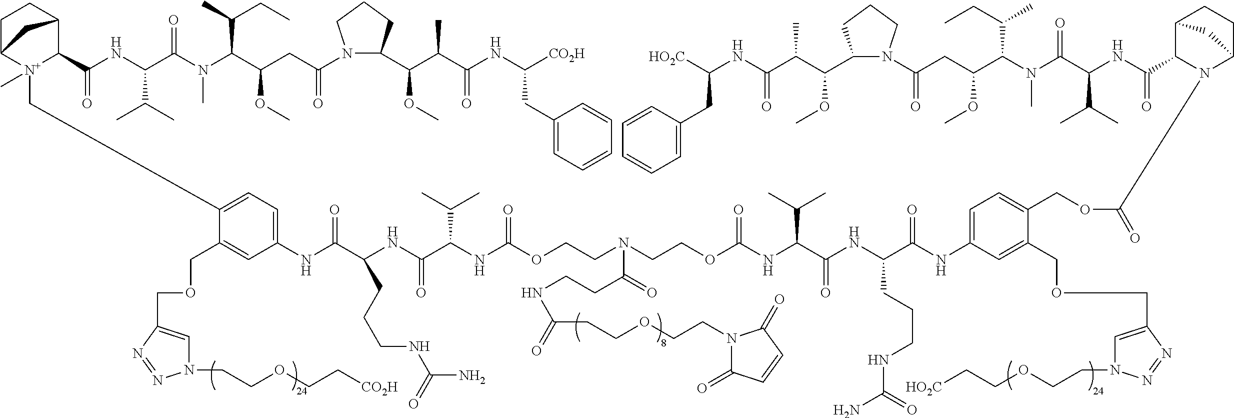 |
| P3- L19-P4 | 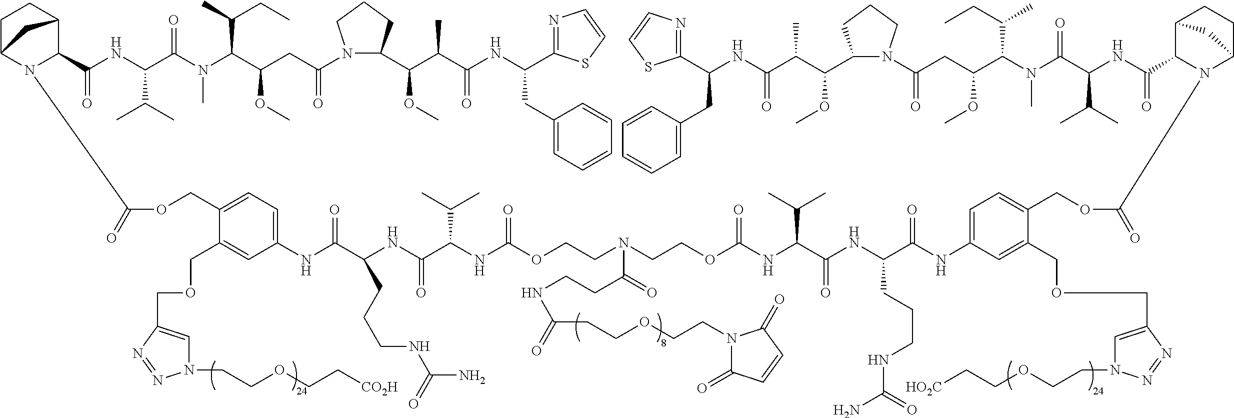 |
| P4- L19-P4 | 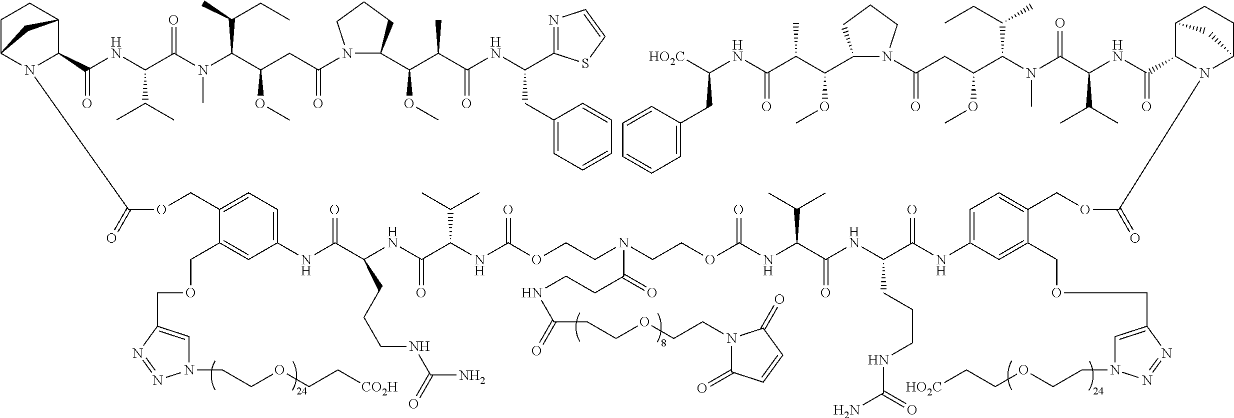 |
| P1- L20-P1 | 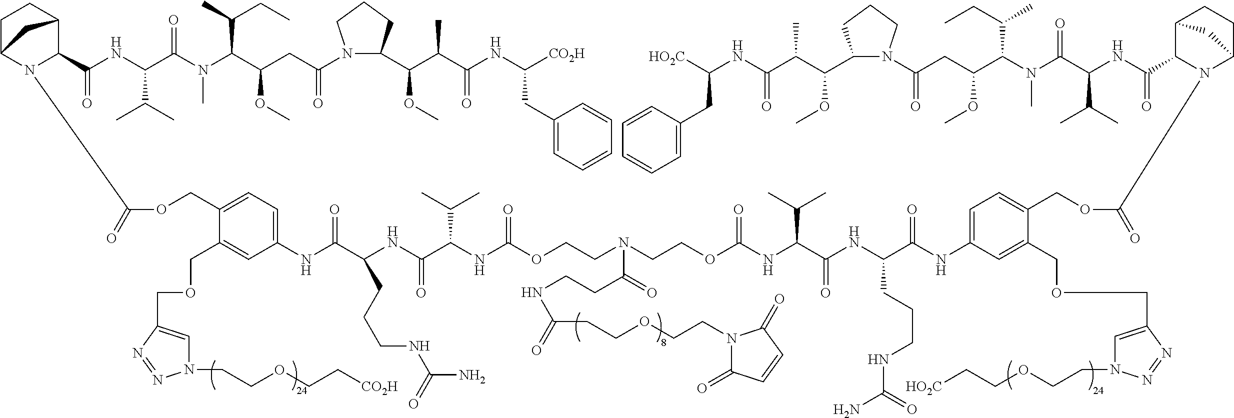 |
| P1- L20-P2 | 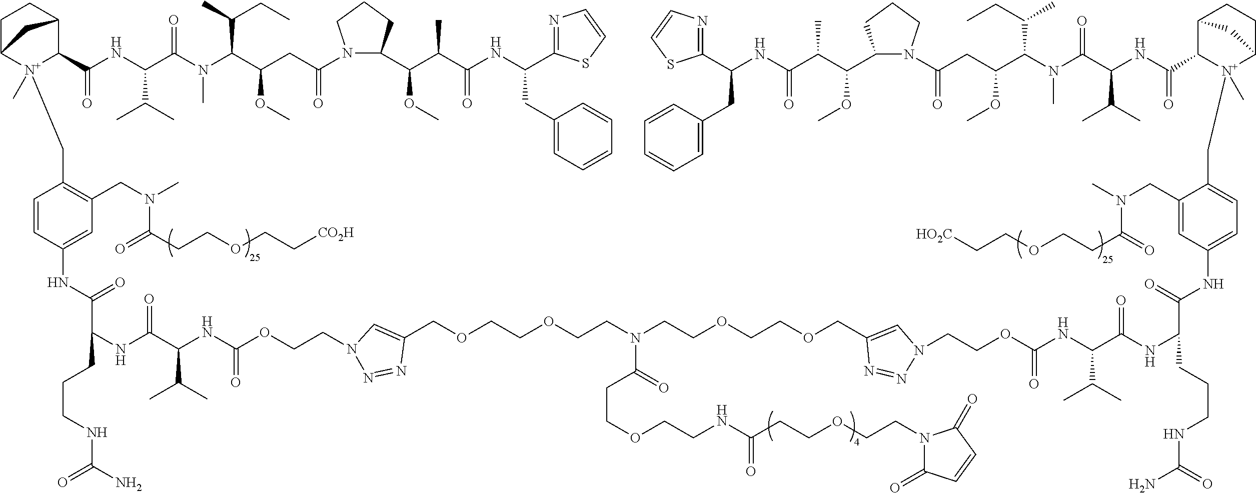 |
| P2- L20-P2 | 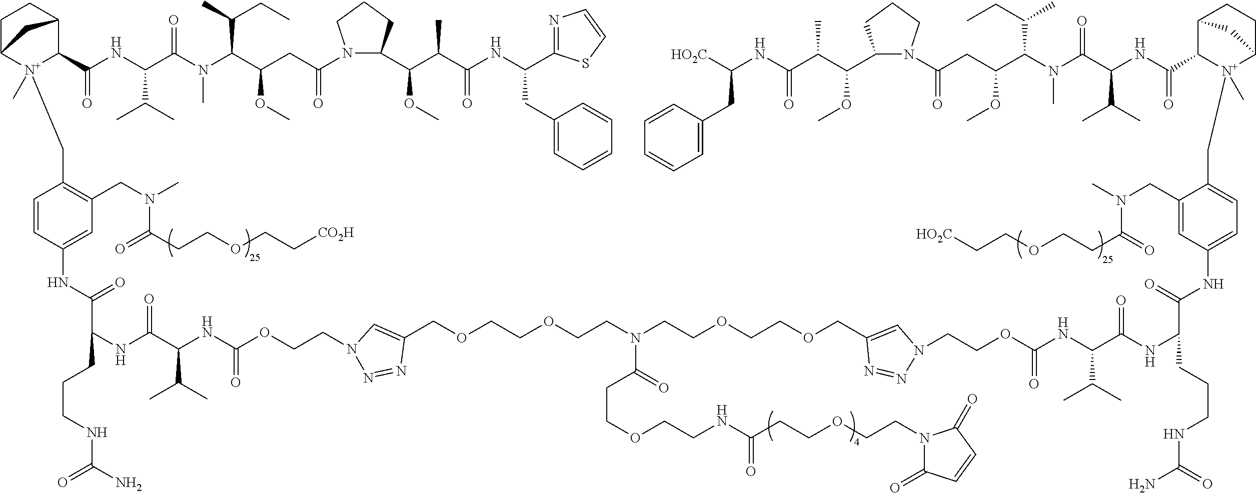 |
| P3- L20-P3 | 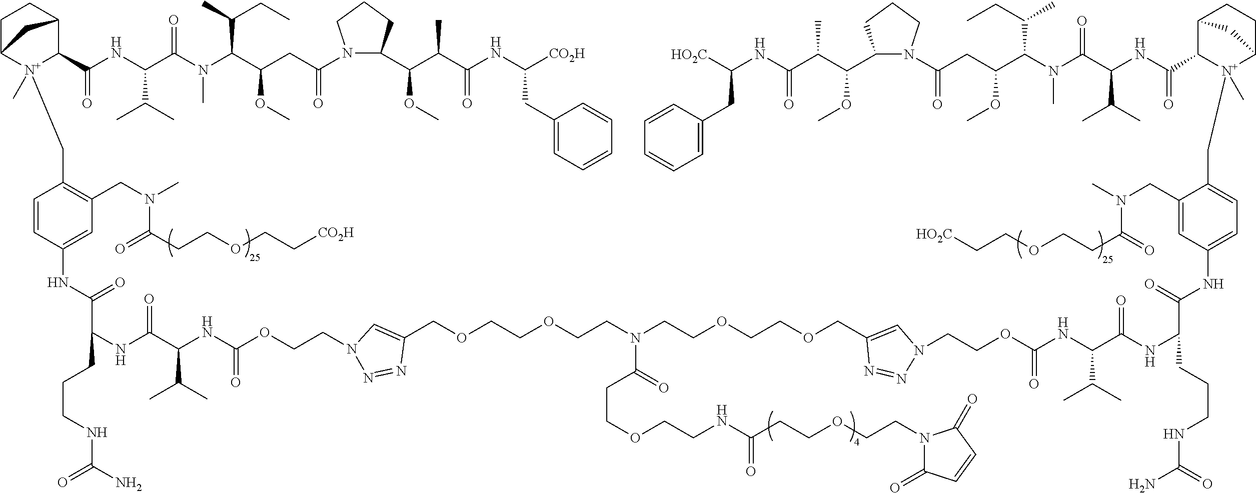 |
| P3- L20-P4 | 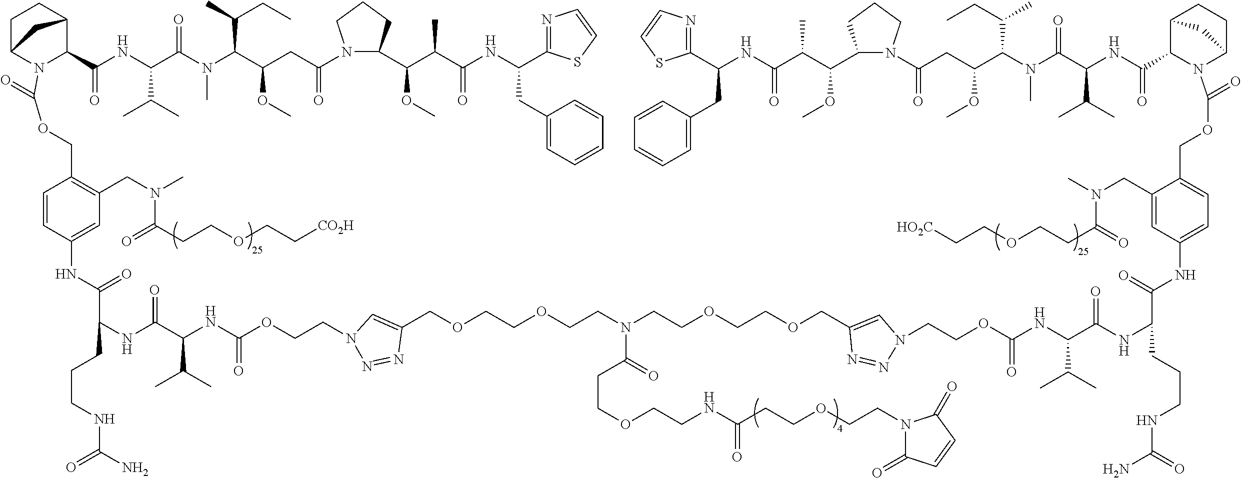 |
| P4- L20-P4 | 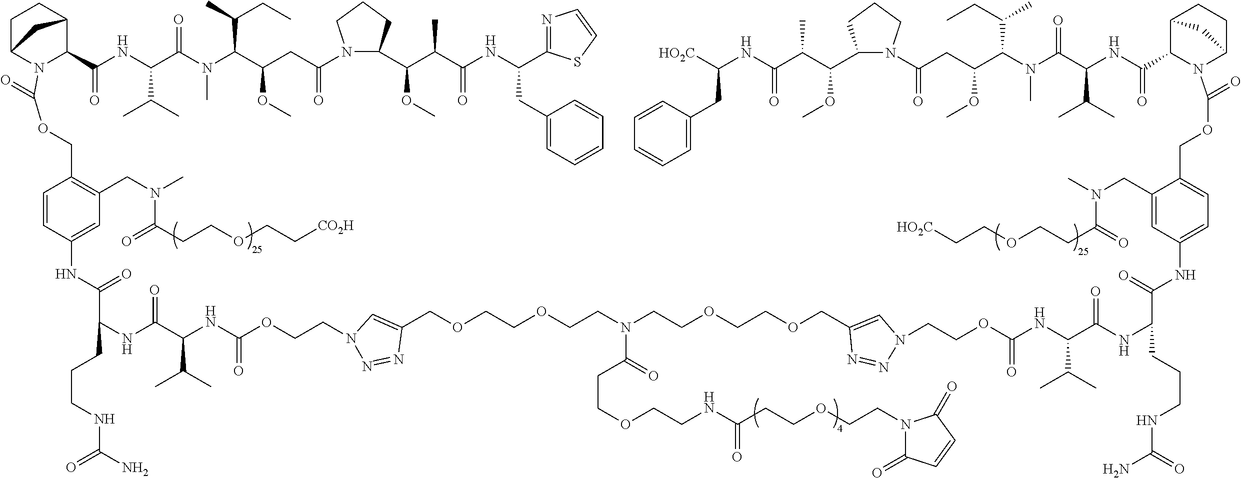 |
| P1- L21-P1 | 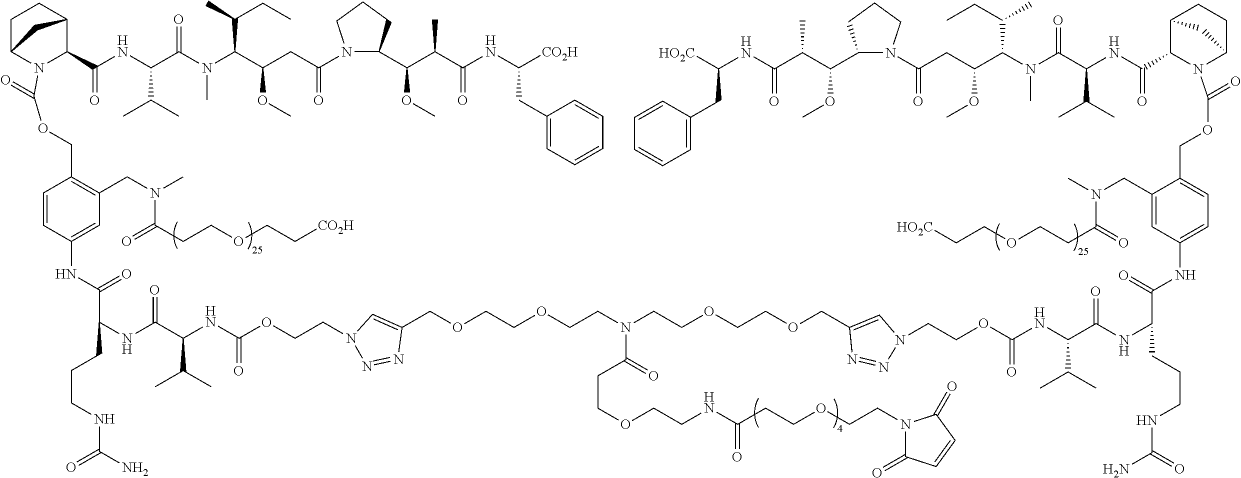 |
| P1- L21-P2 | 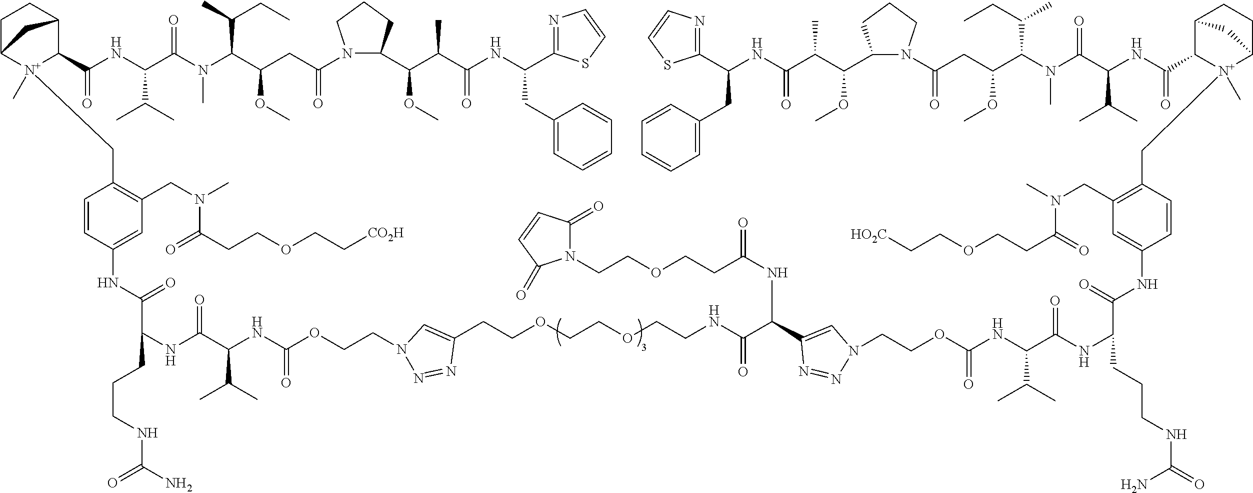 |
| P2- L21-P1 | 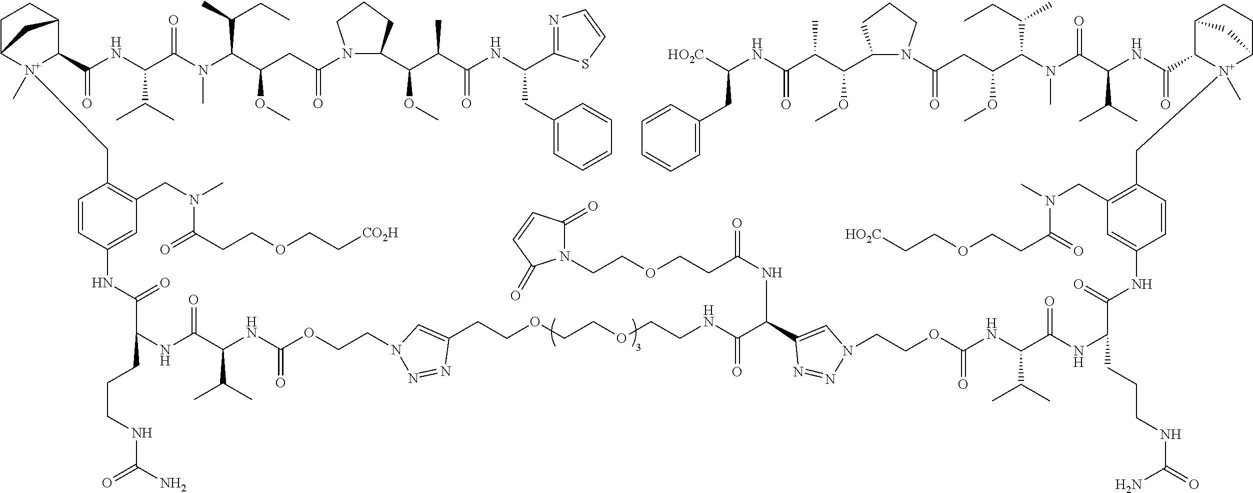 |
| P2- L21-P2 | 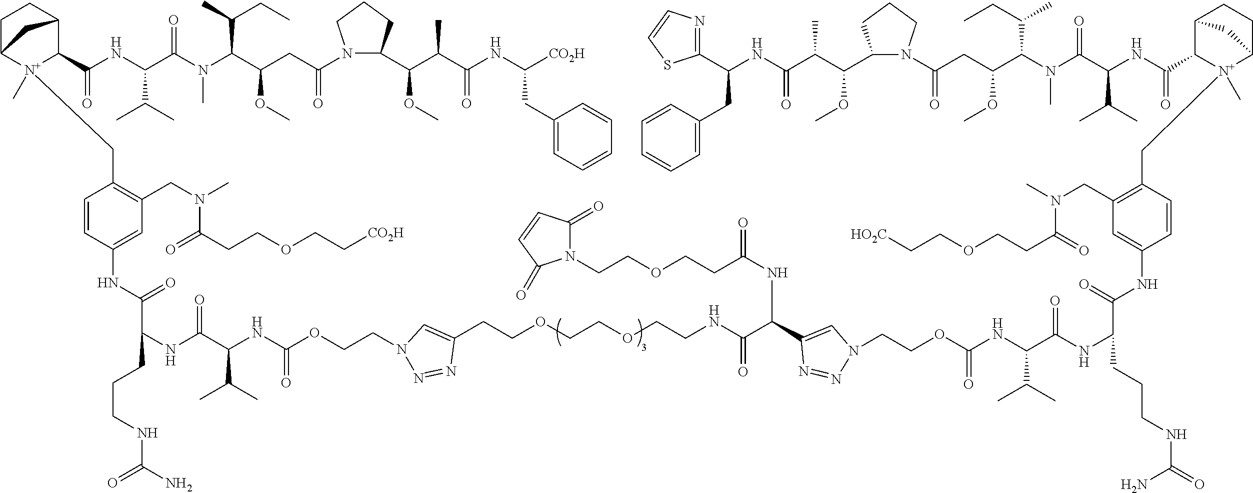 |
| P3- L21-P3 | 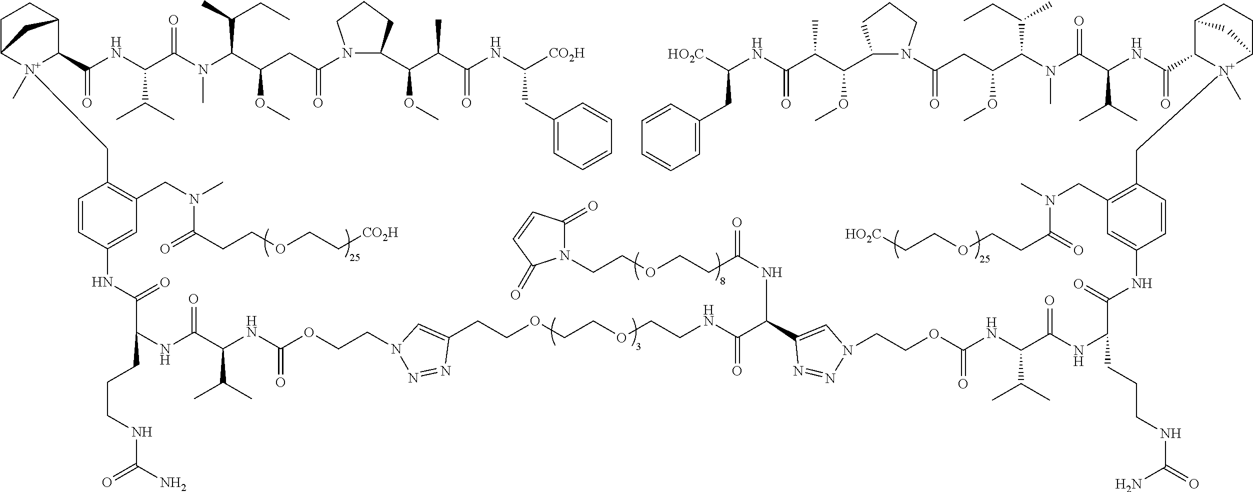 |
| P3- L21-P4 | 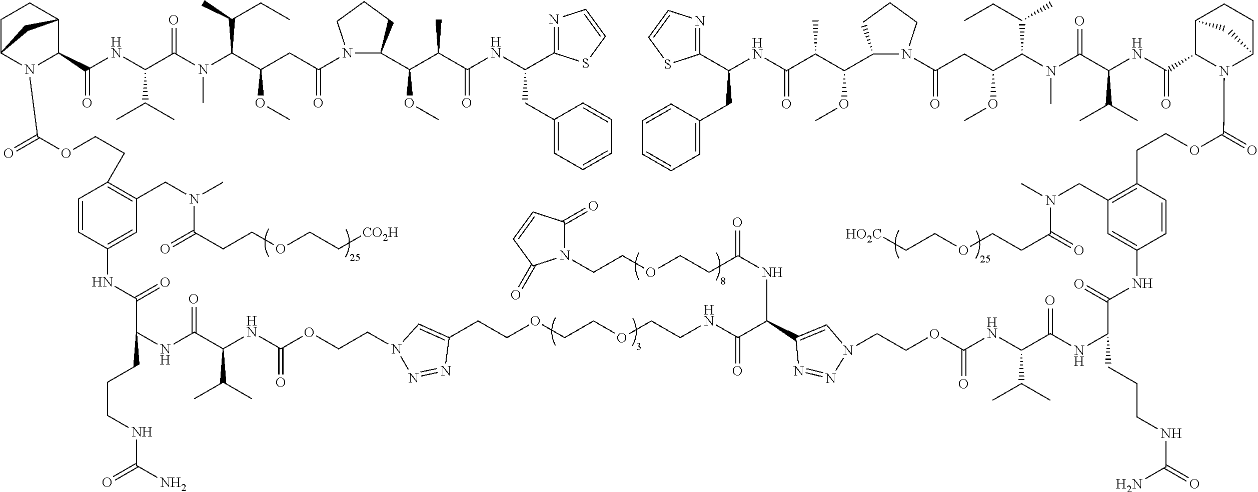 |
| P4- L21-P3 | 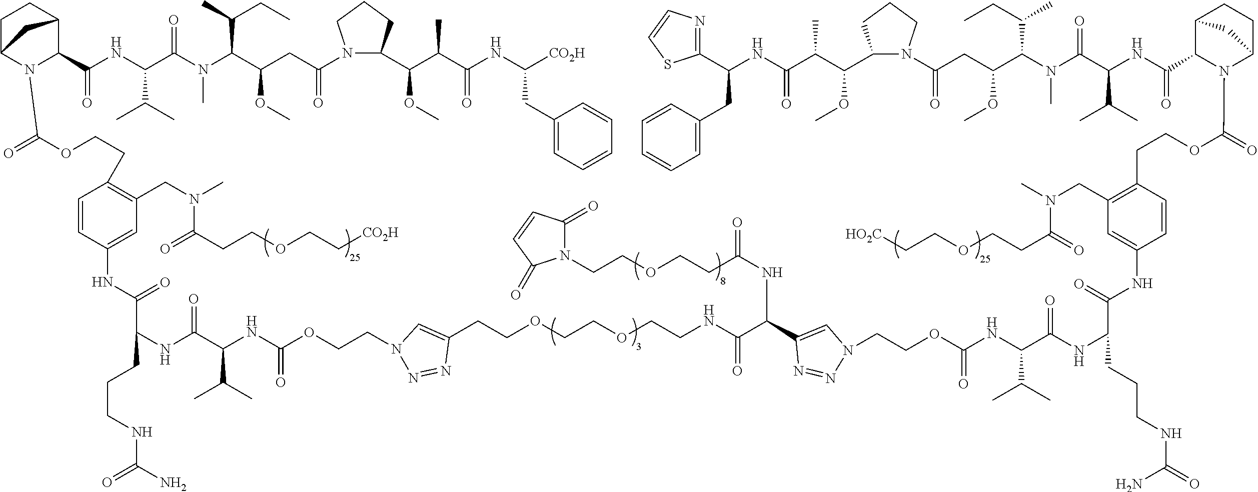 |
| P4- L21-P4 | 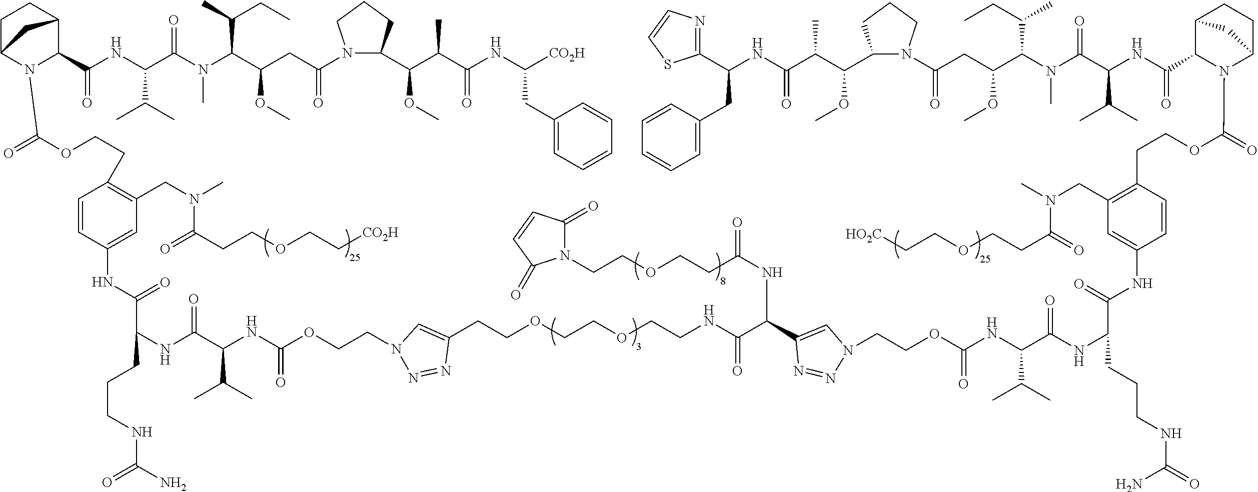 |
| P1- L22-P1 | 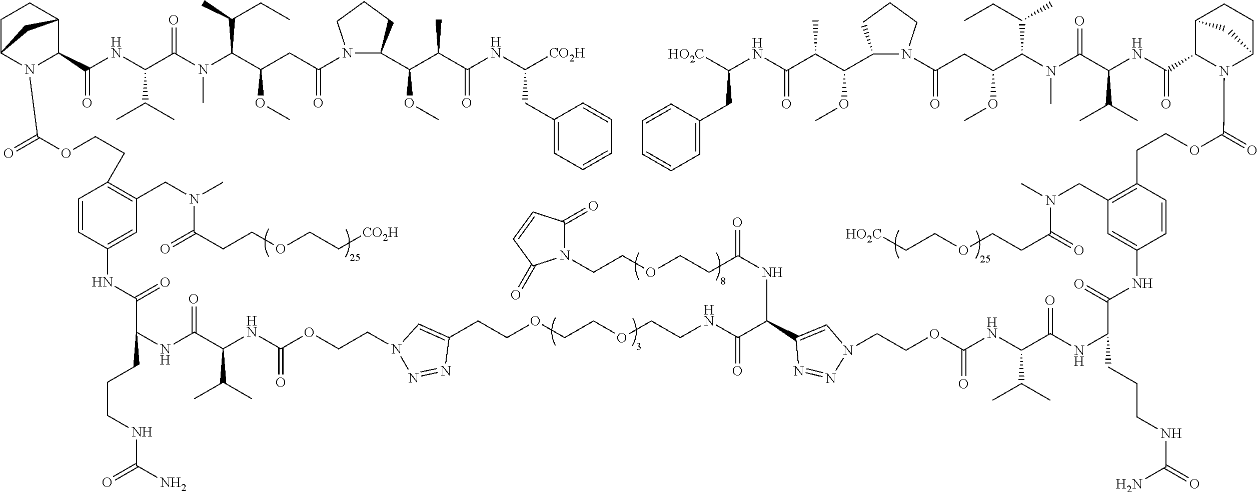 |
| P1- L22-P2 | 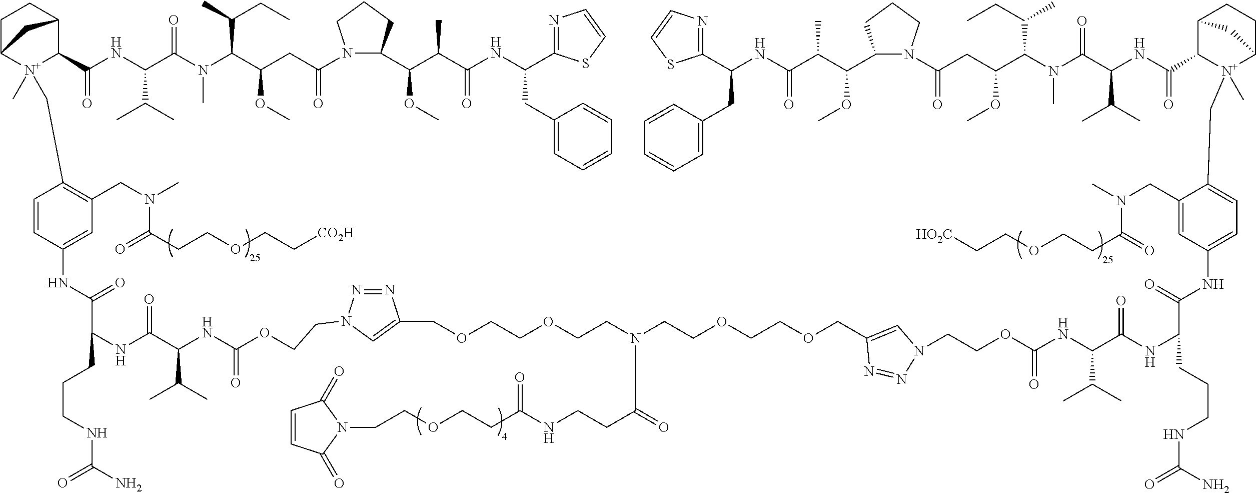 |
| P2- L22-P2 | 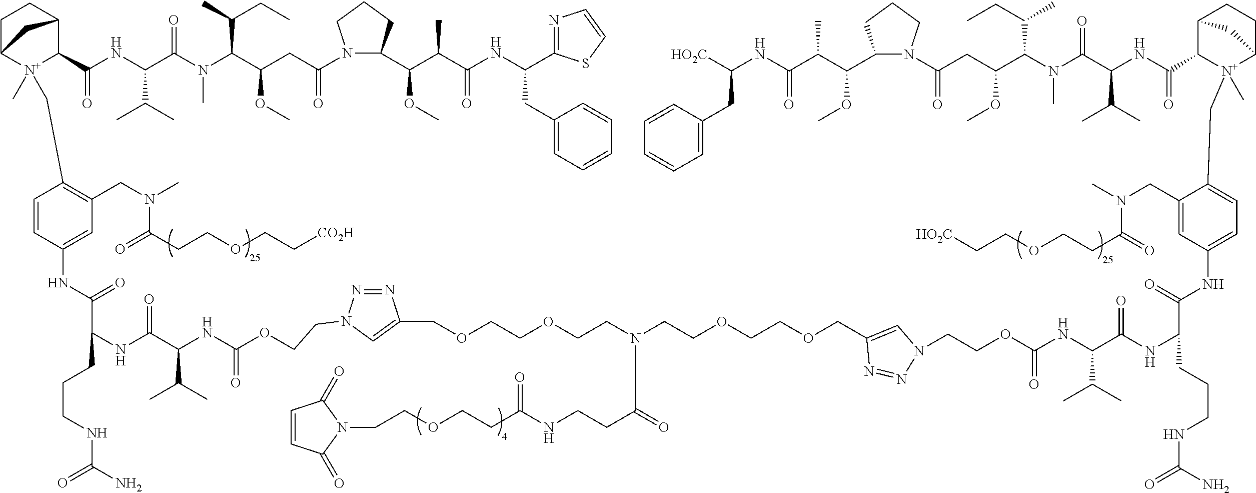 |
| P3- L22-P3 | 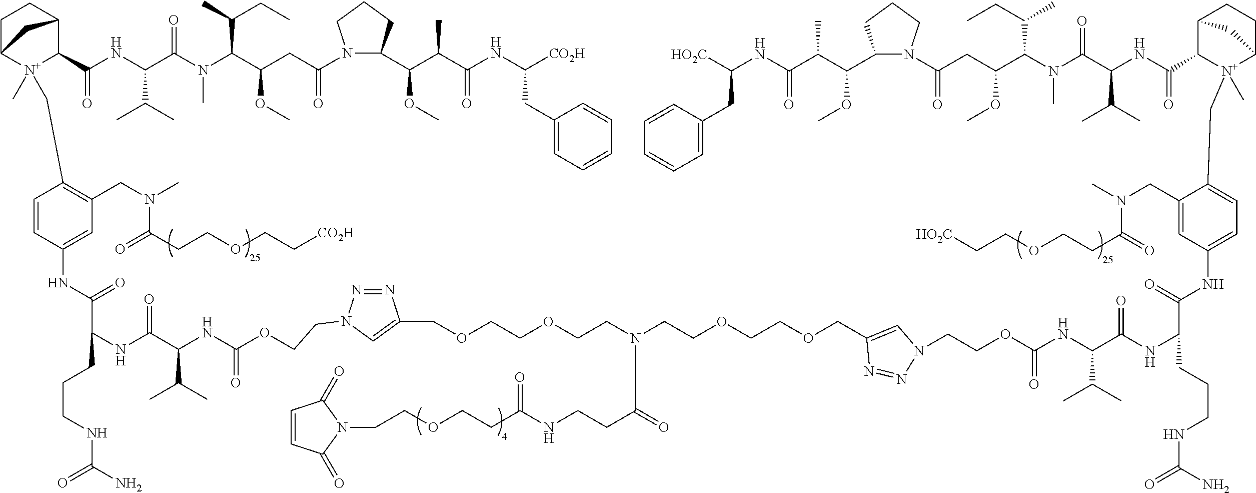 |
| P3- L22-P4 | 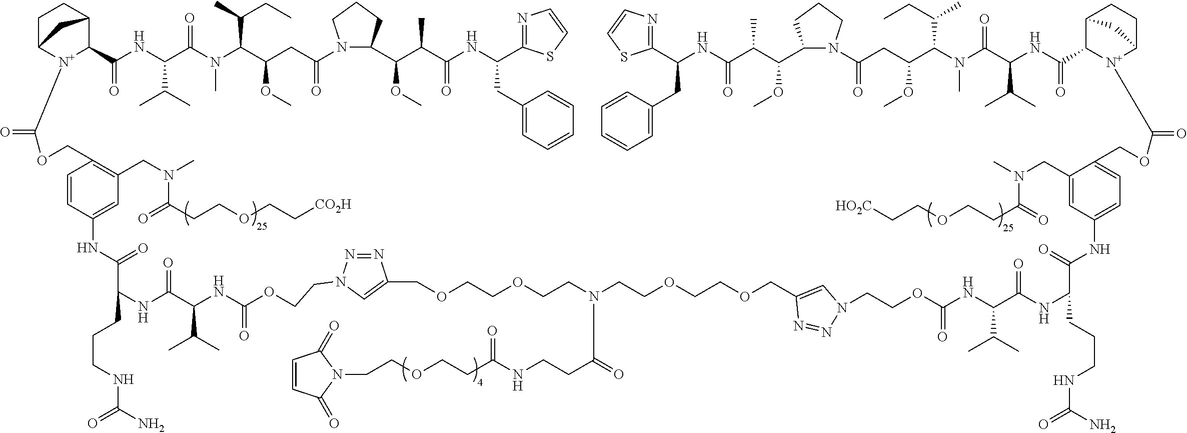 |
| P4- L22-P4 | 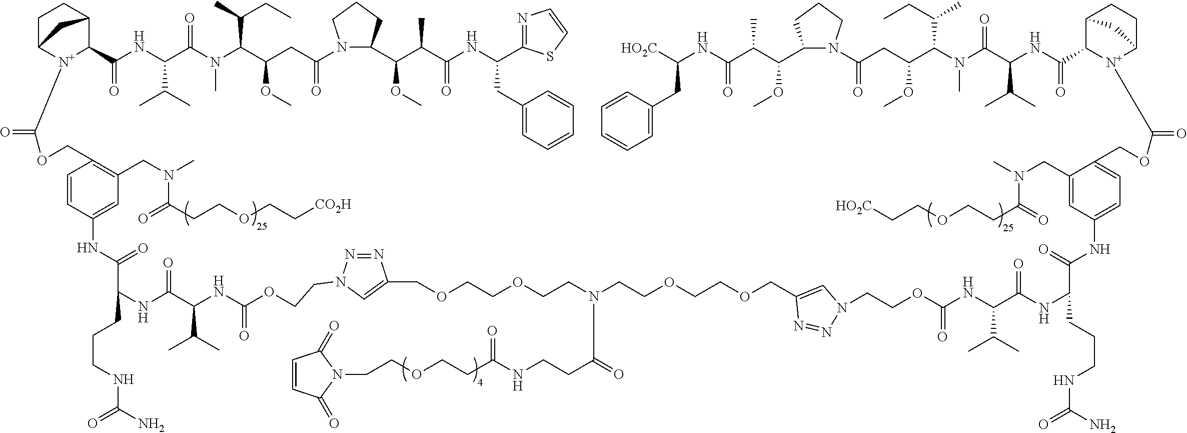 |
| P1- L23-P1 | 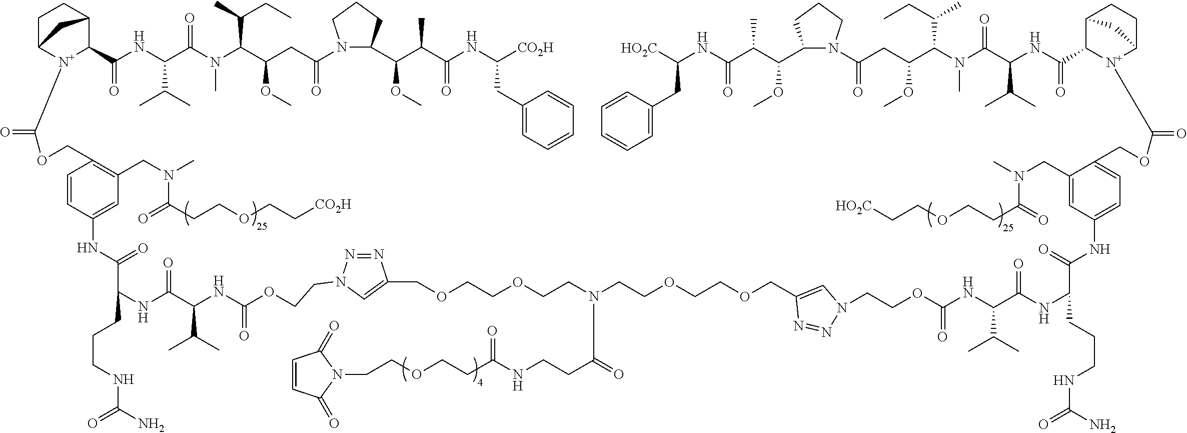 |
| P1- L23-P2 | 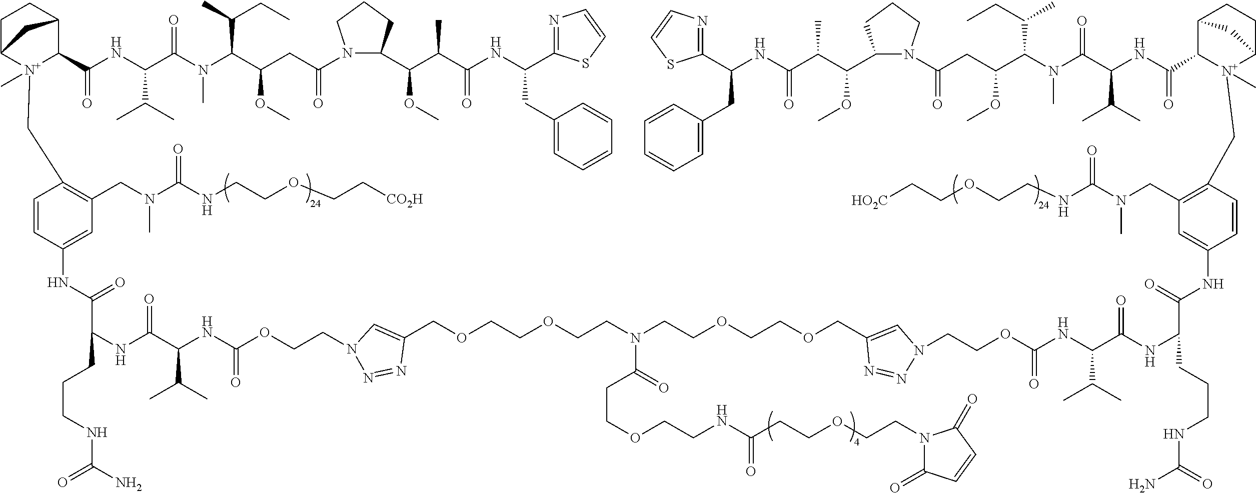 |
| P2- L23-P2 | 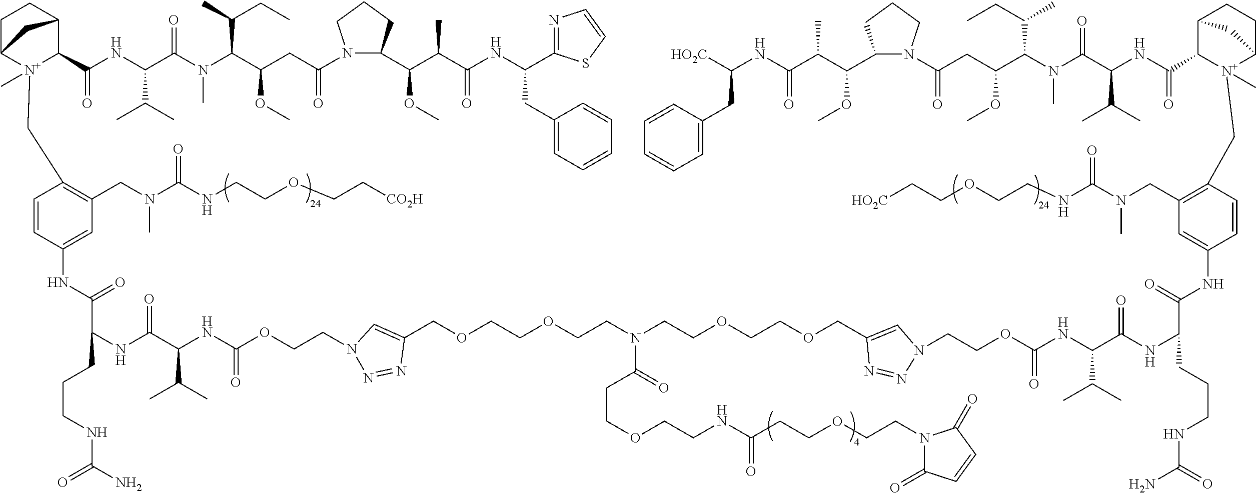 |
| P3- L23-P3 | 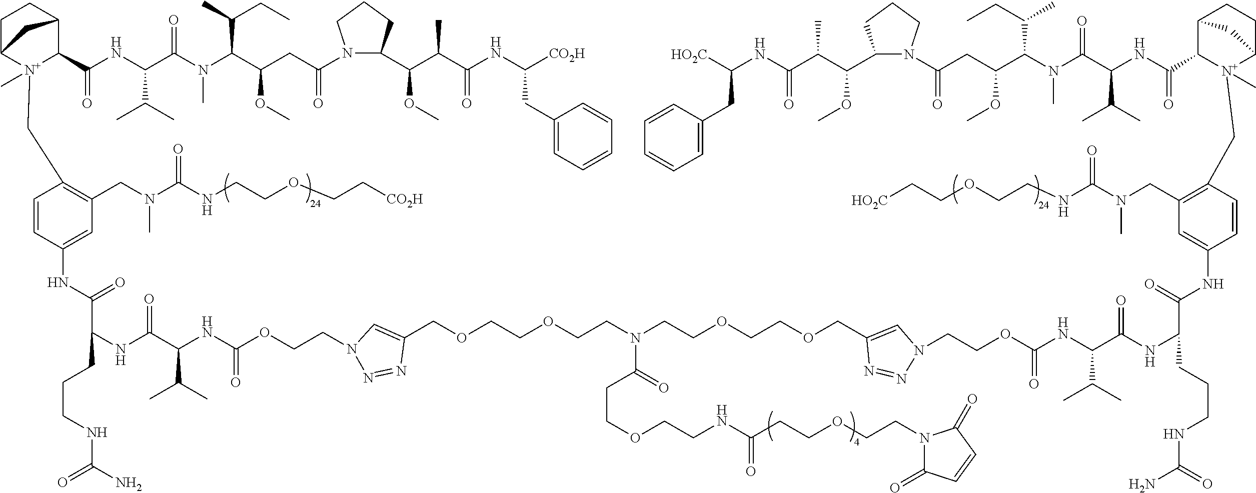 |
| P3- L23-P4 | 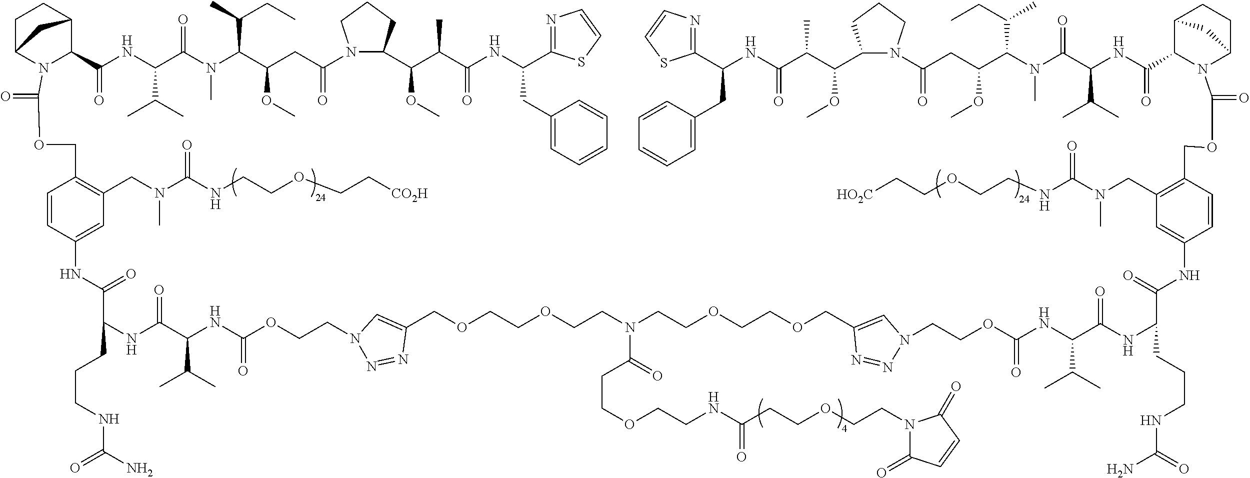 |
| P4- L23-P4 | 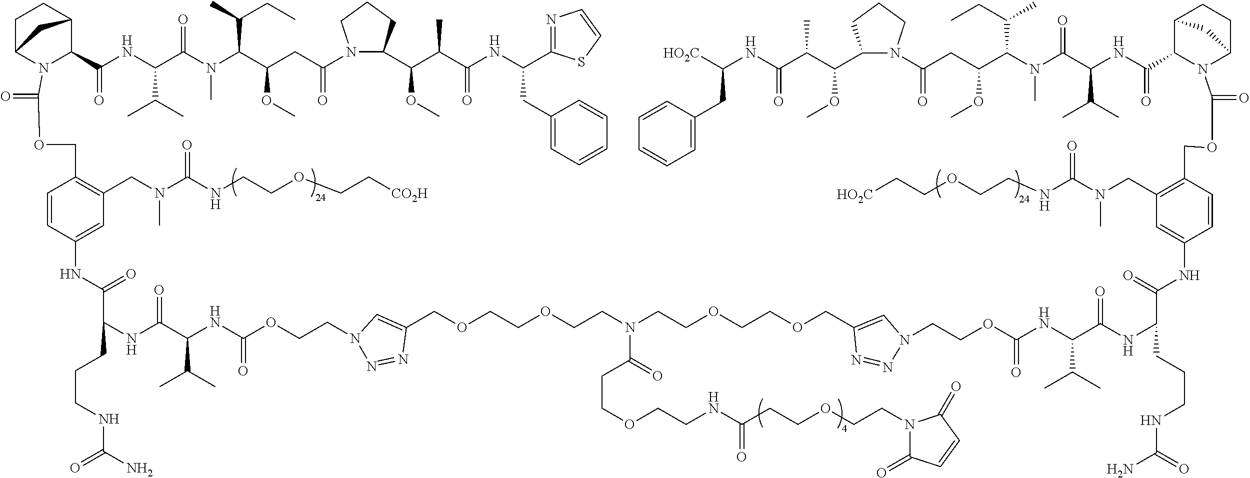 |
| P1- L24-P1 | 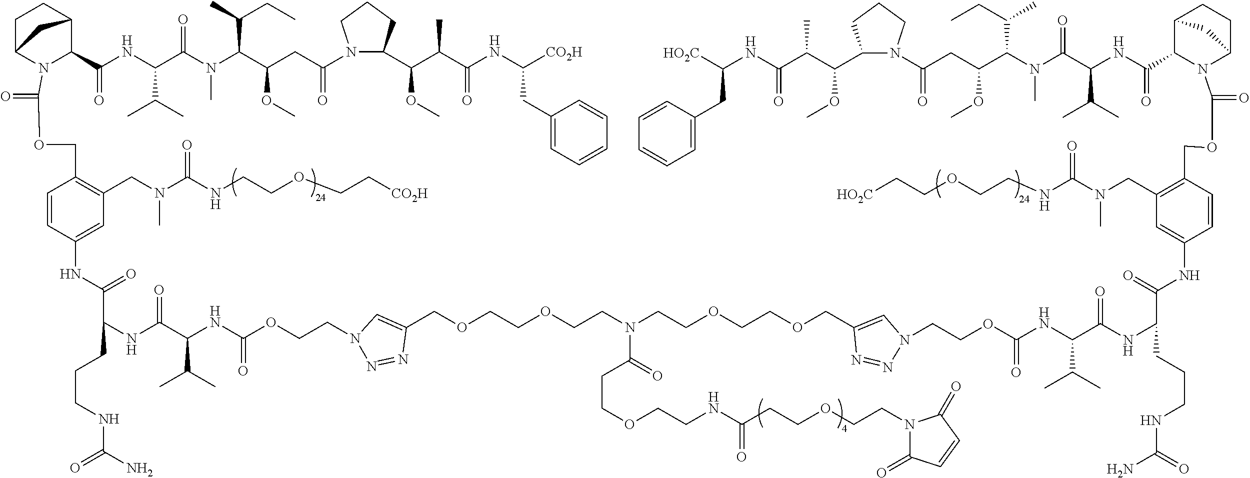 |
| P1- L24-P2 | 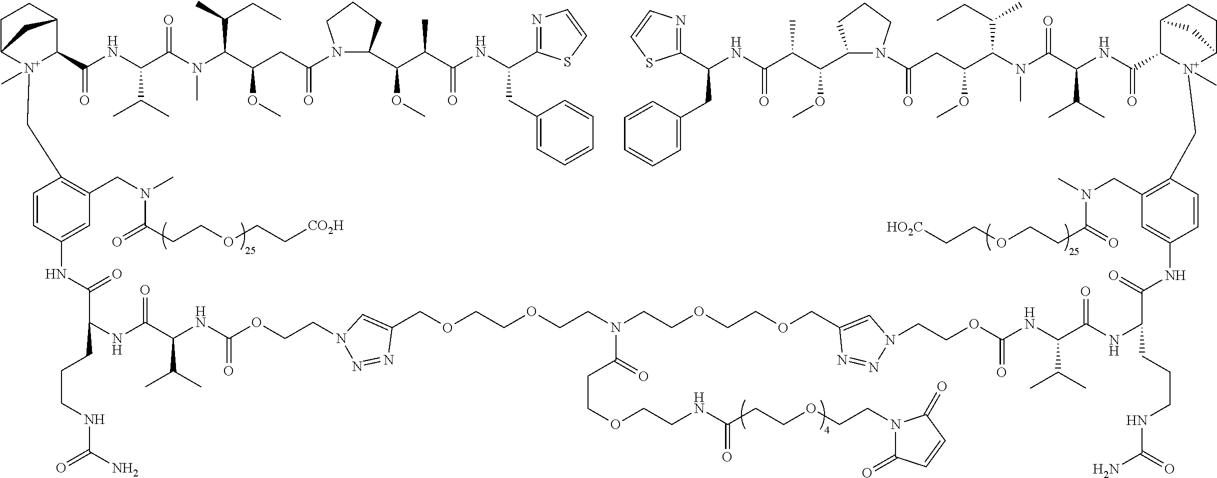 |
| P2- L24-P2 | 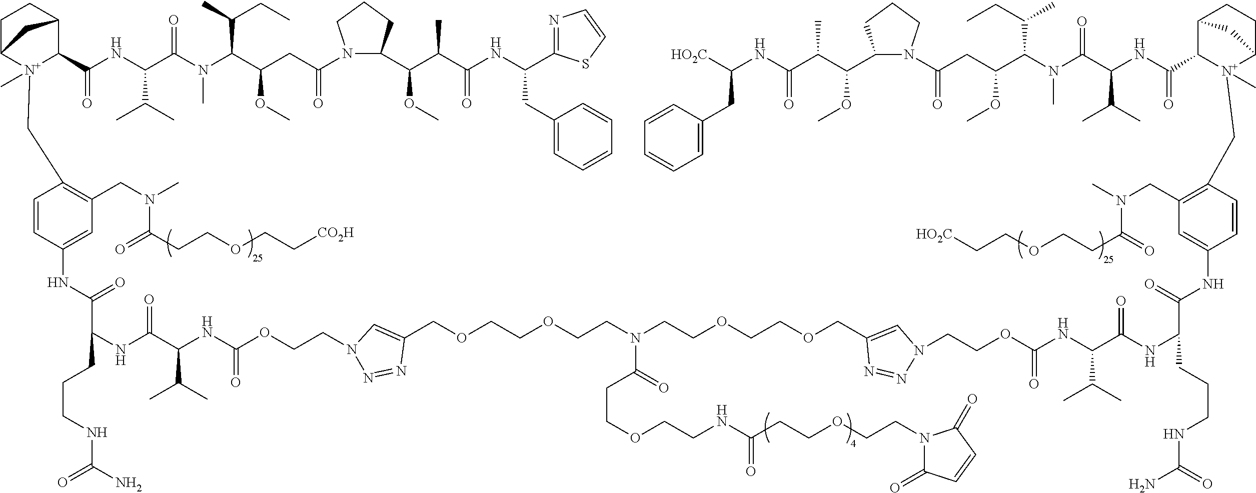 |
| P3- L24-P3 | 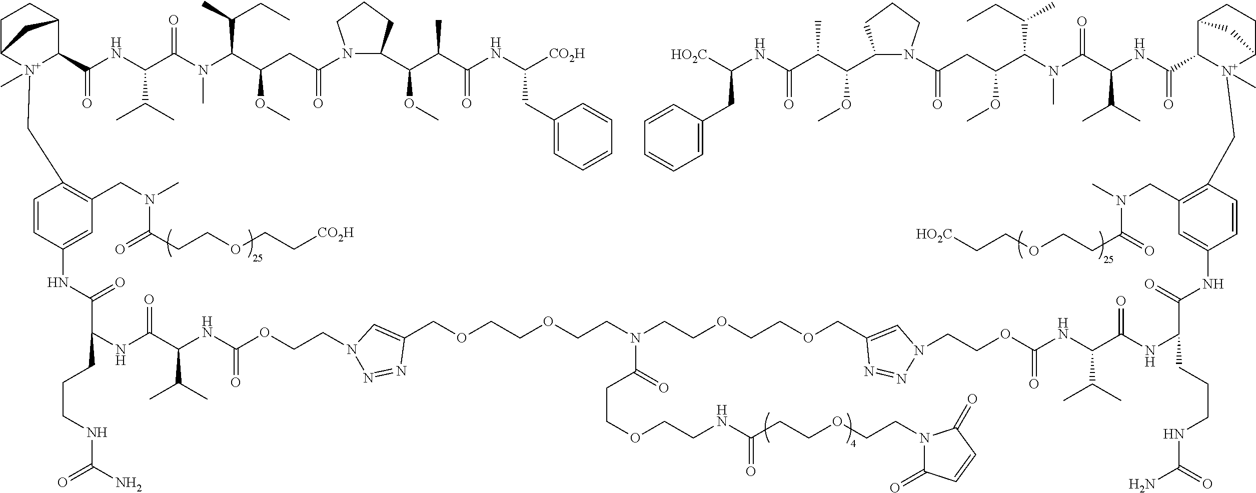 |
| P3- L24-P4 | 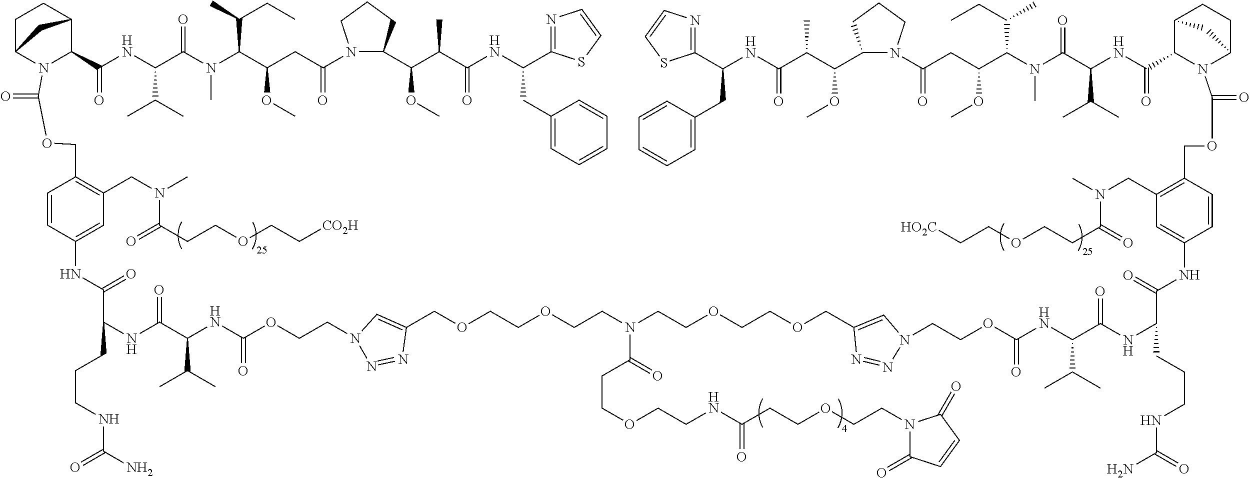 |
| P4- L24-P4 | 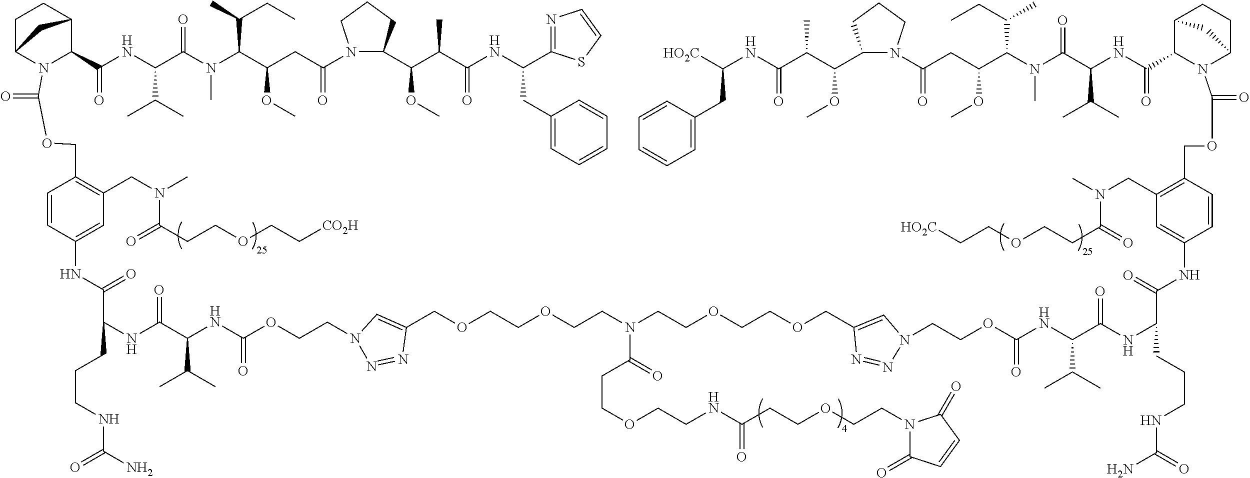 |
| P1- L25-P1 | 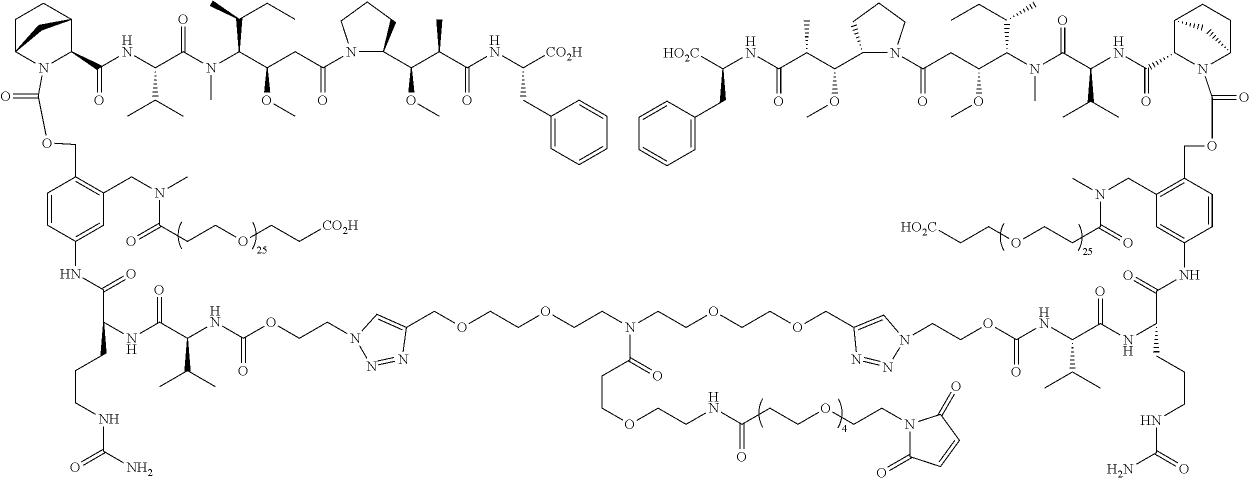 |
| P1- L25-P2 | 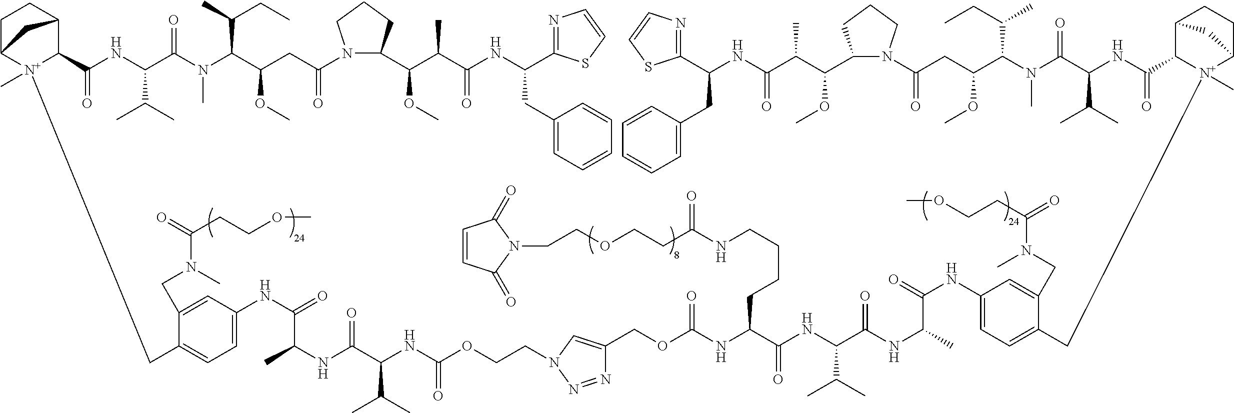 |
| P2- L25-P1 | 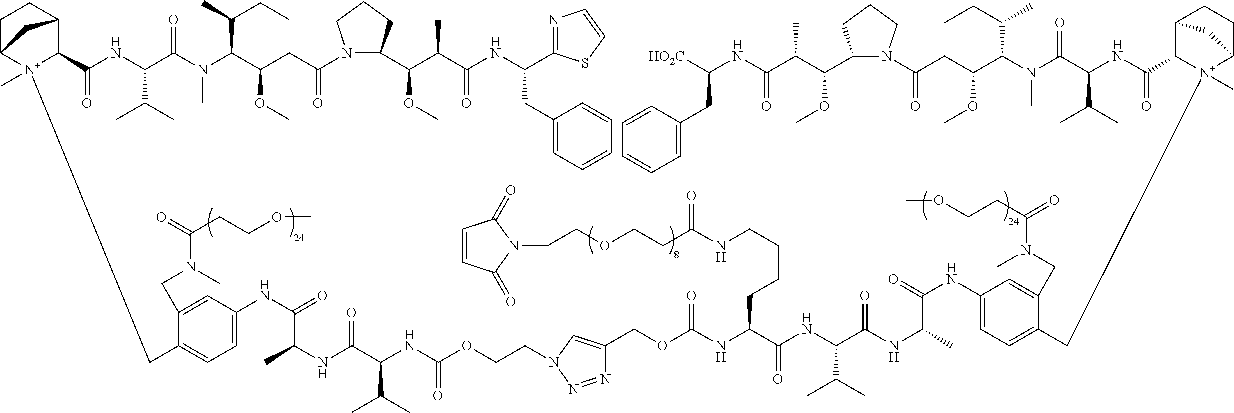 |
| P2- L25-P2 | 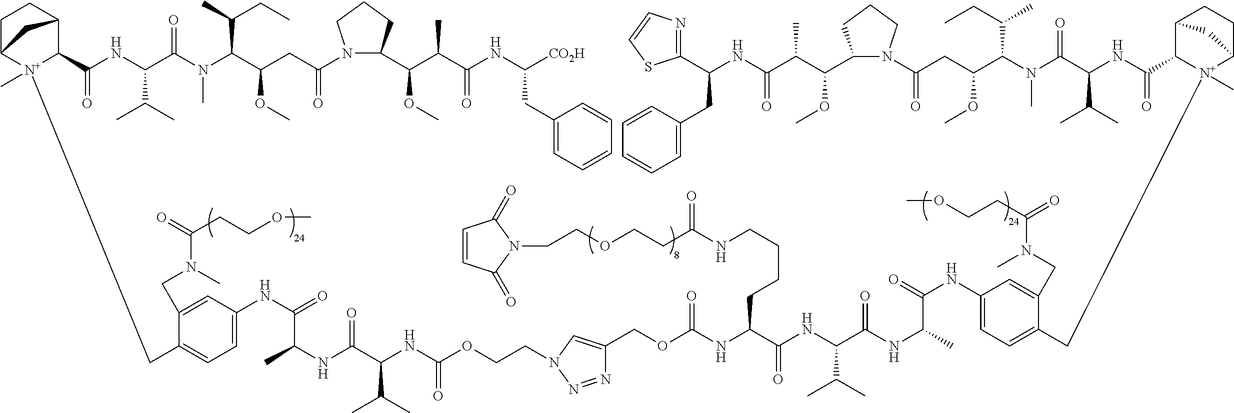 |
| P3- L25-P3 | 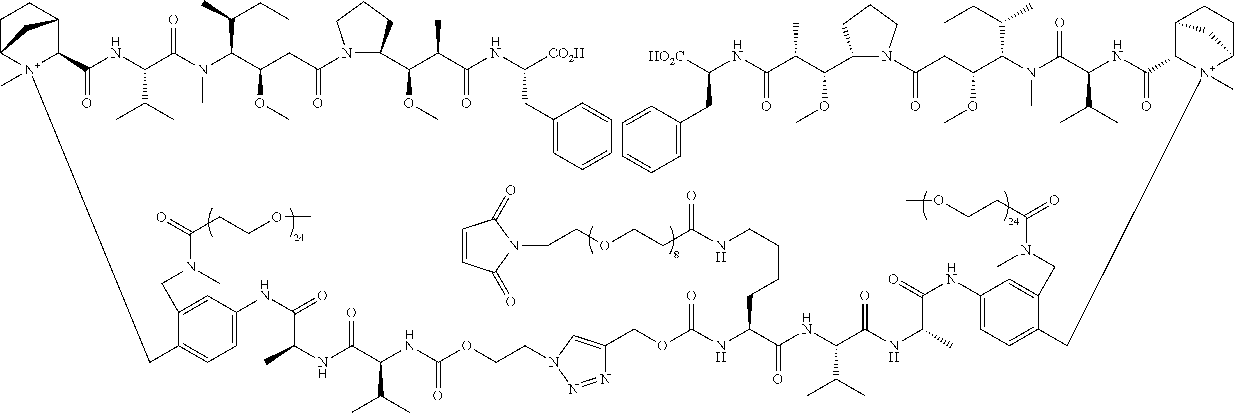 |
| P3- L25-P4 | 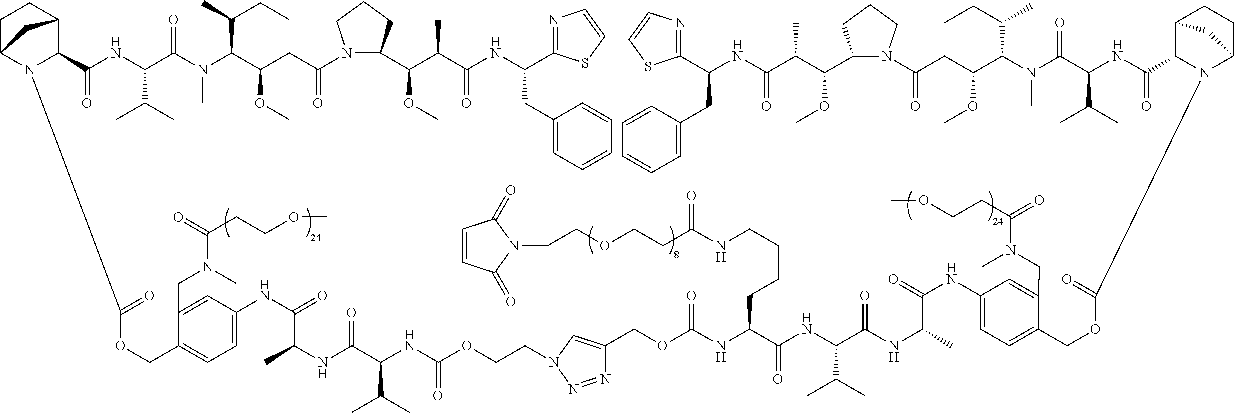 |
| P4- L25-P3 | 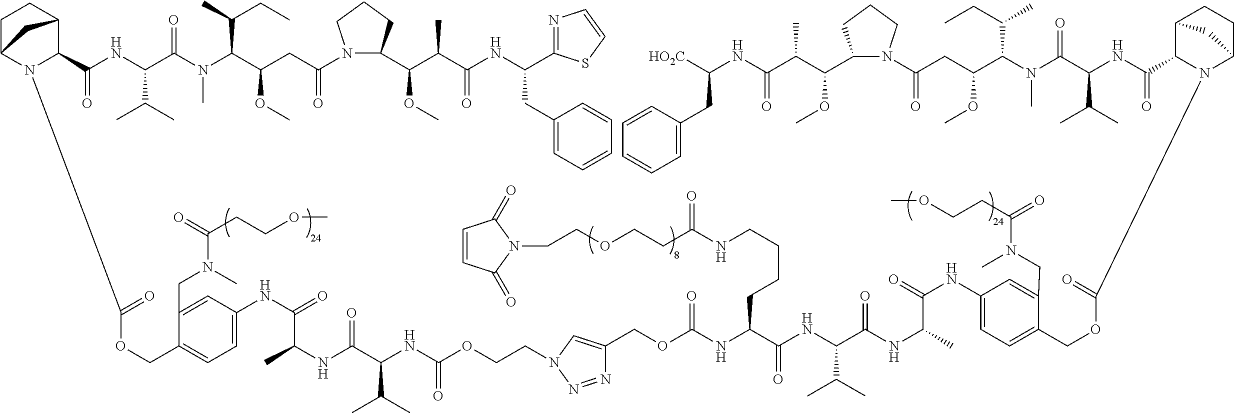 |
| P4- L25-P4 | 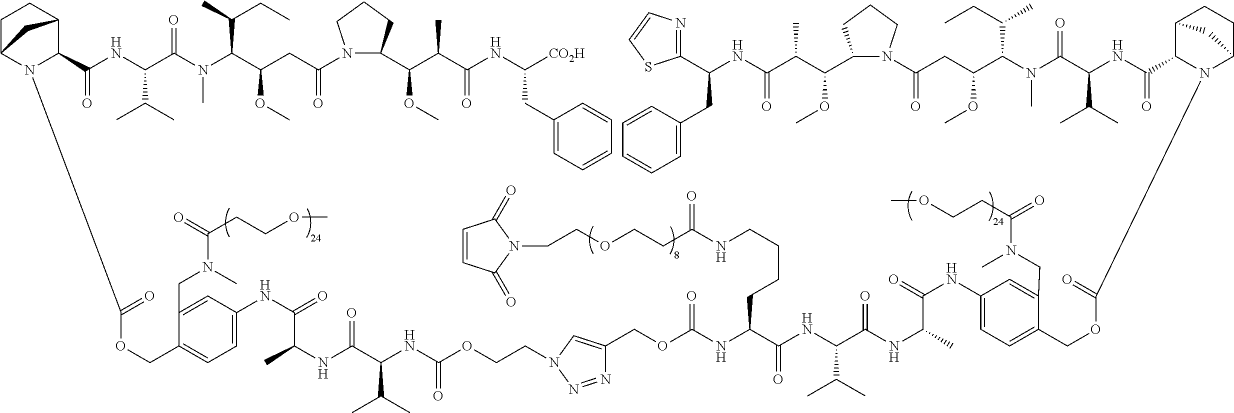 |
| P1- L26-P1 | 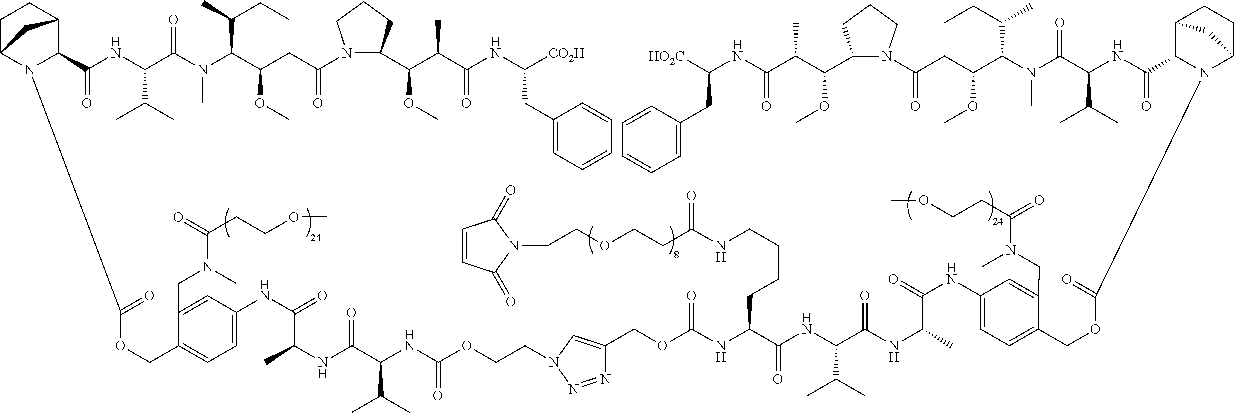 |
| P1- L26-P2 | 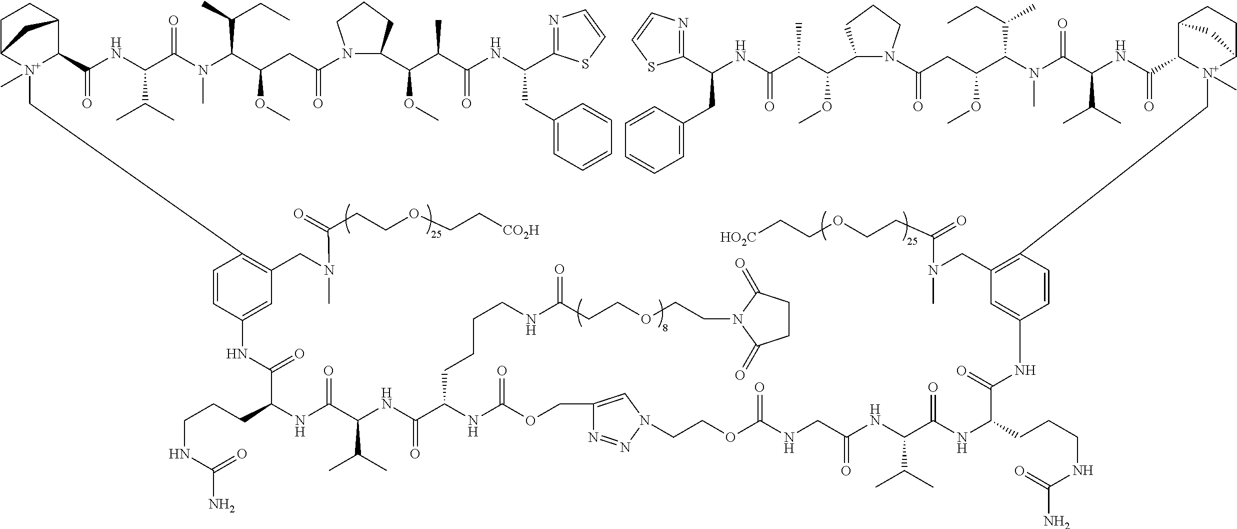 |
| P2- L26-P1 | 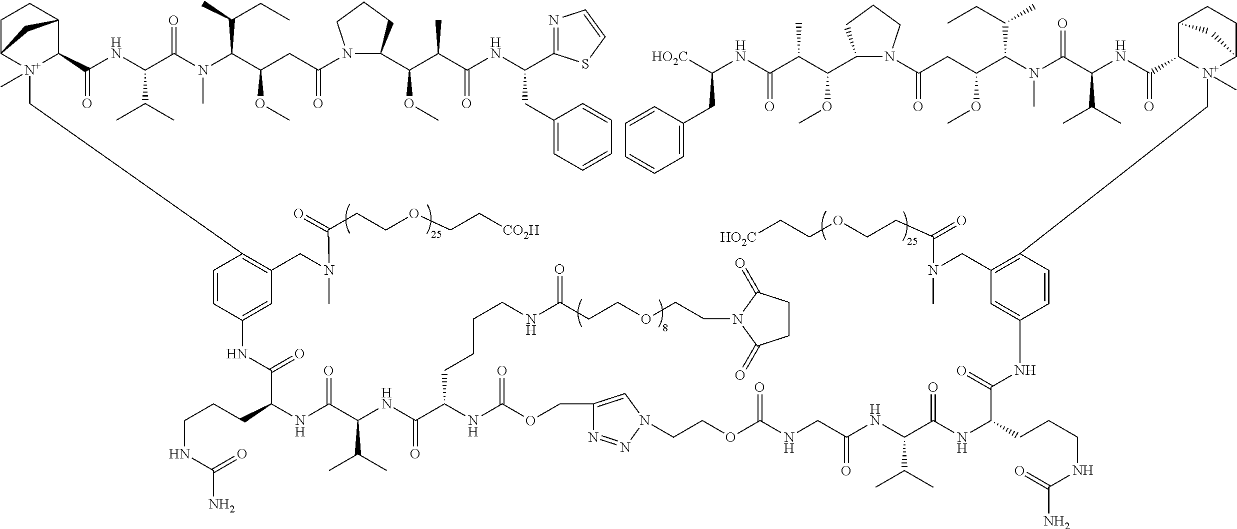 |
| P2- L26-P2 | 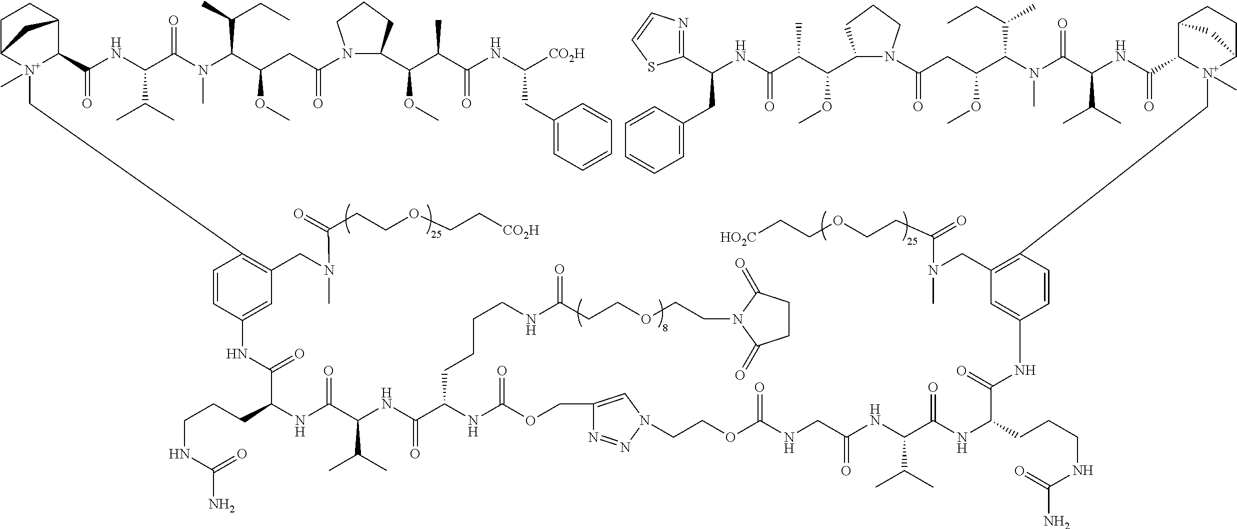 |
| P3- L26-P3 | 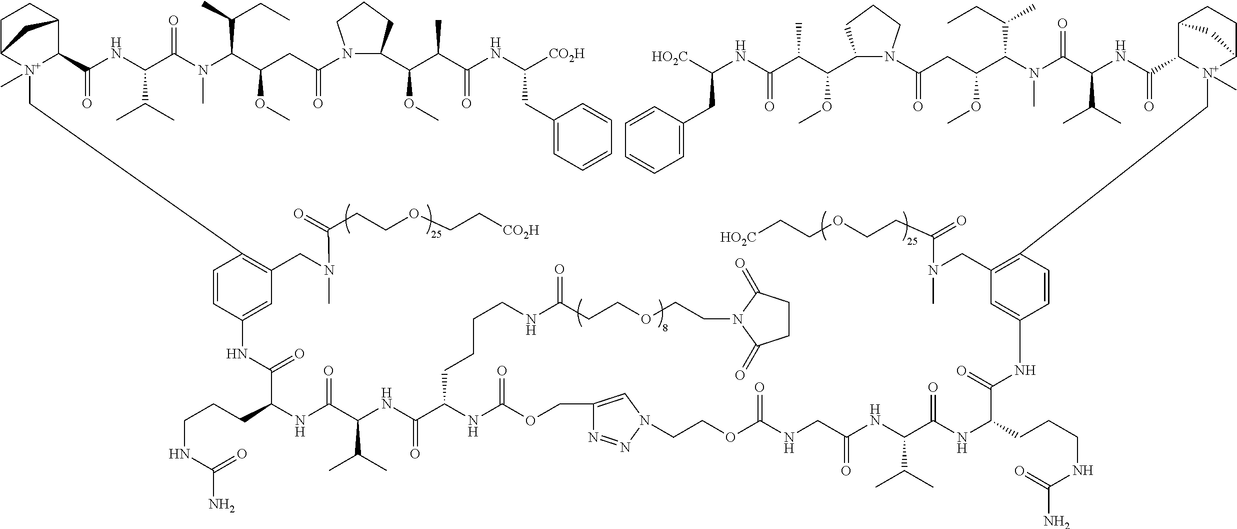 |
| P3- L26-P4 | 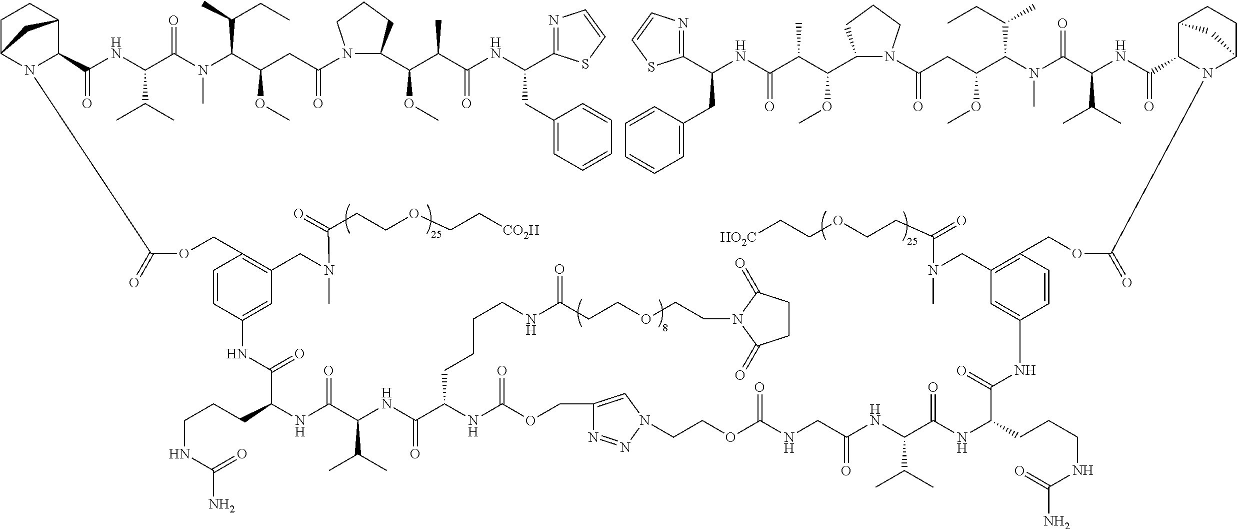 |
| P4- L26-P3 | 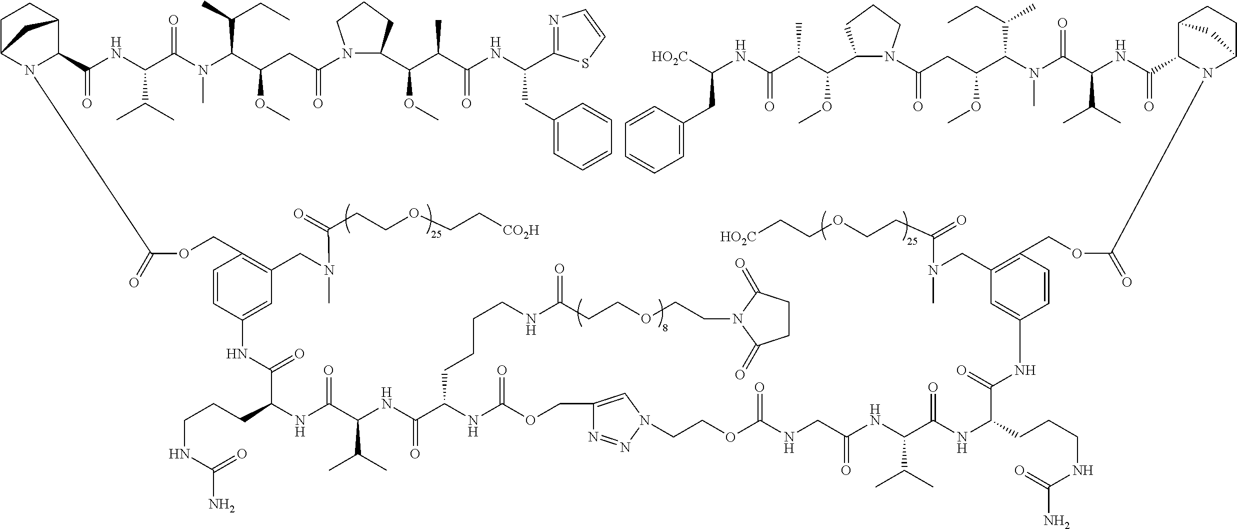 |
| P4- L26-P4 | 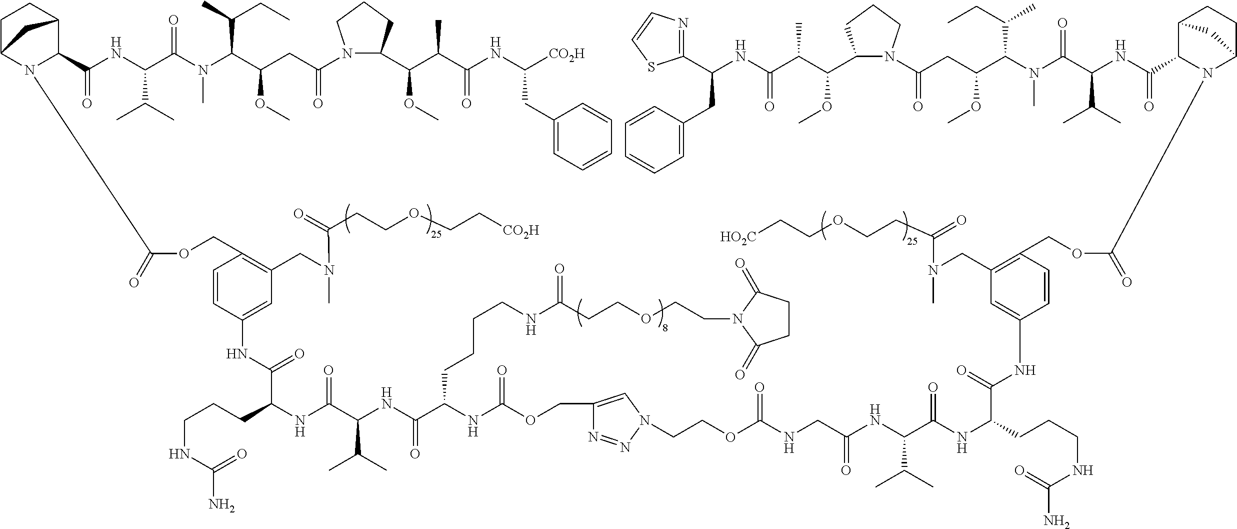 |
| P1- L27-P1 | 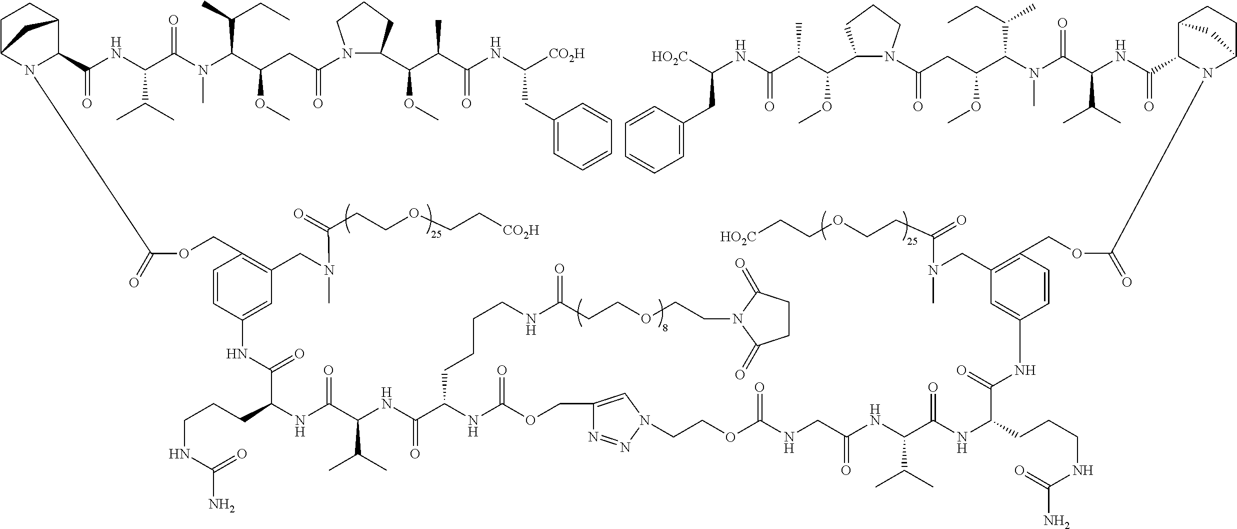 |
| P1- L27-P2 | 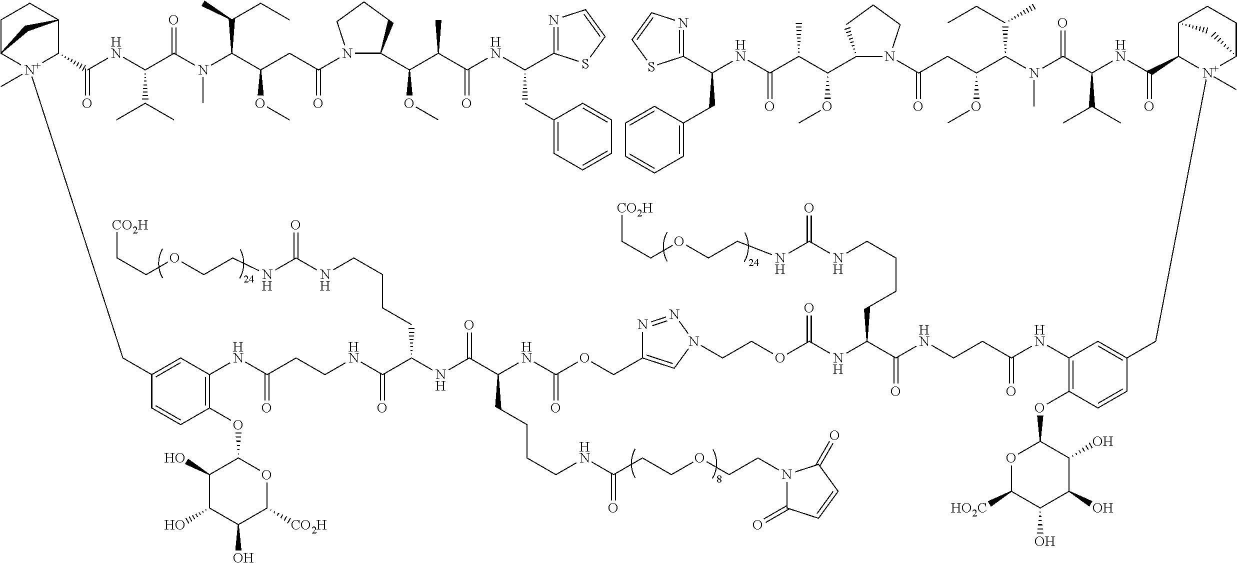 |
| P2- L27-P1 | 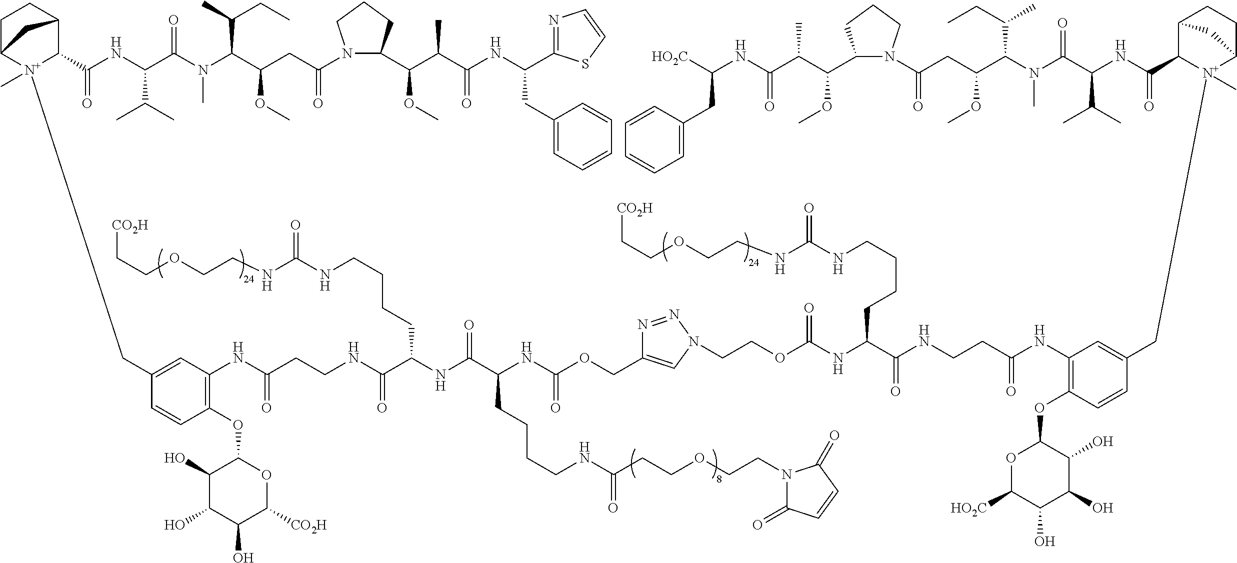 |
| P2- L27-P2 | 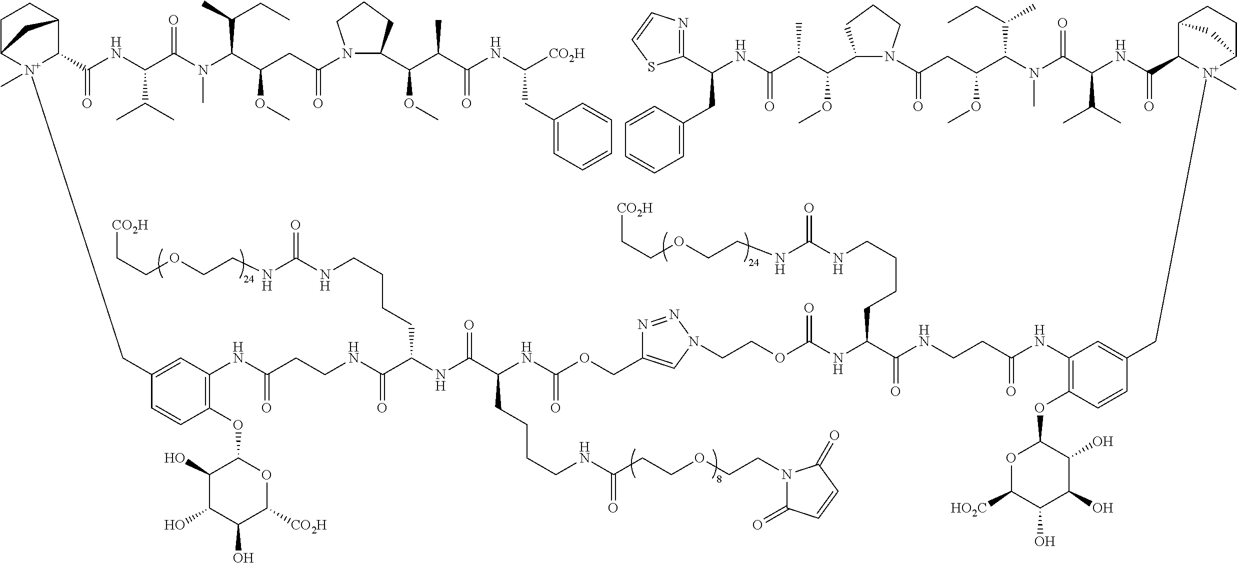 |
| P3- L27-P3 | 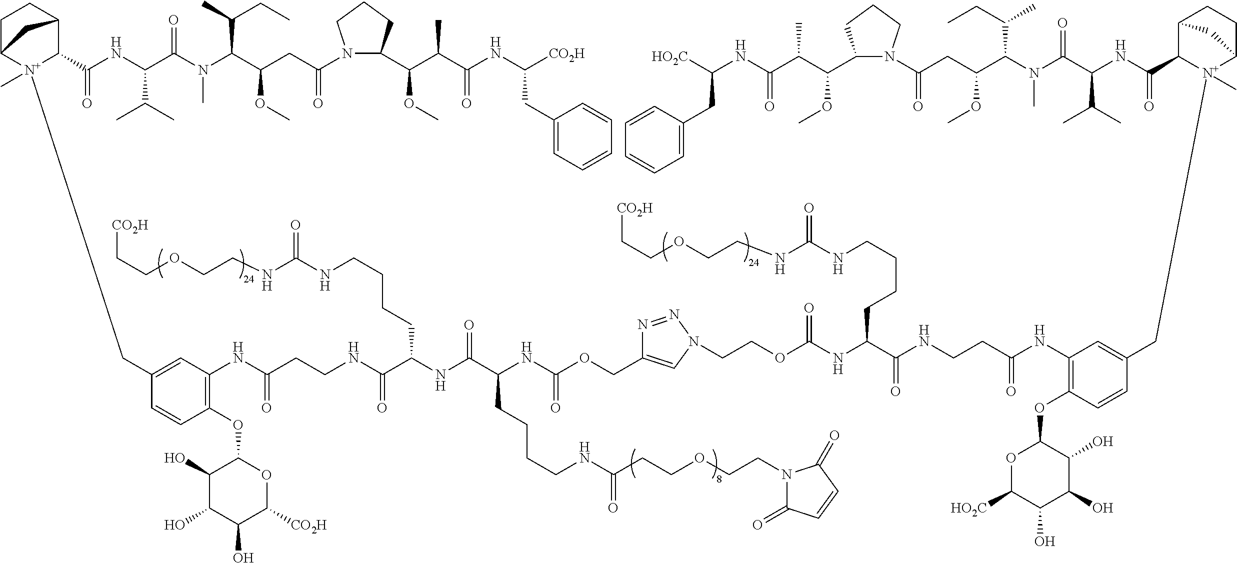 |
| P3- L27-P4 | 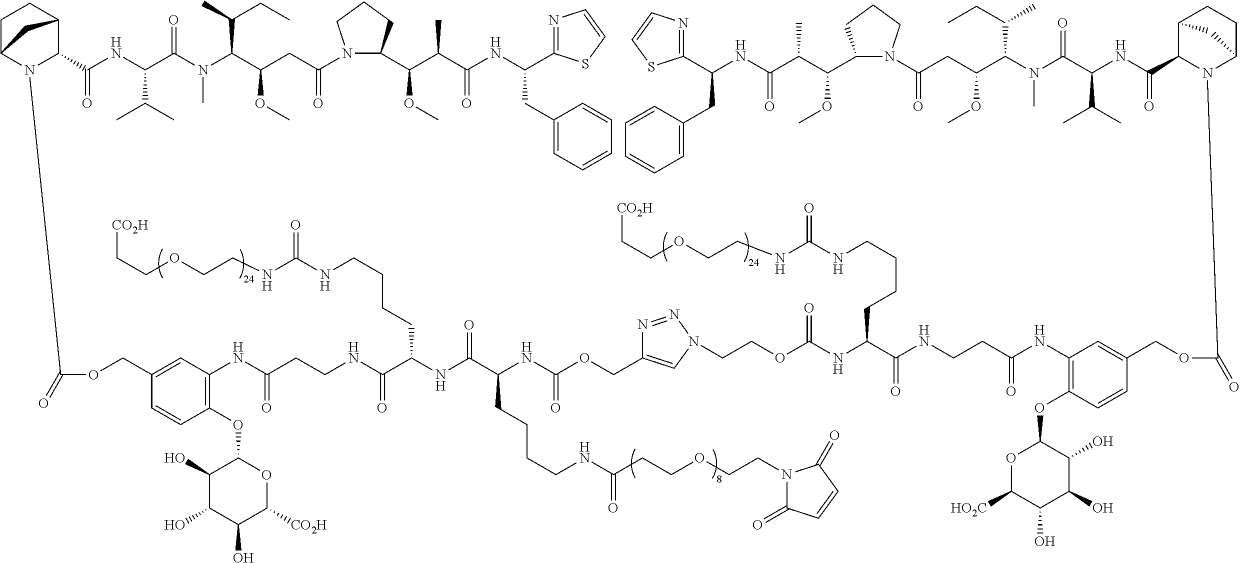 |
| P4- L27-P3 | 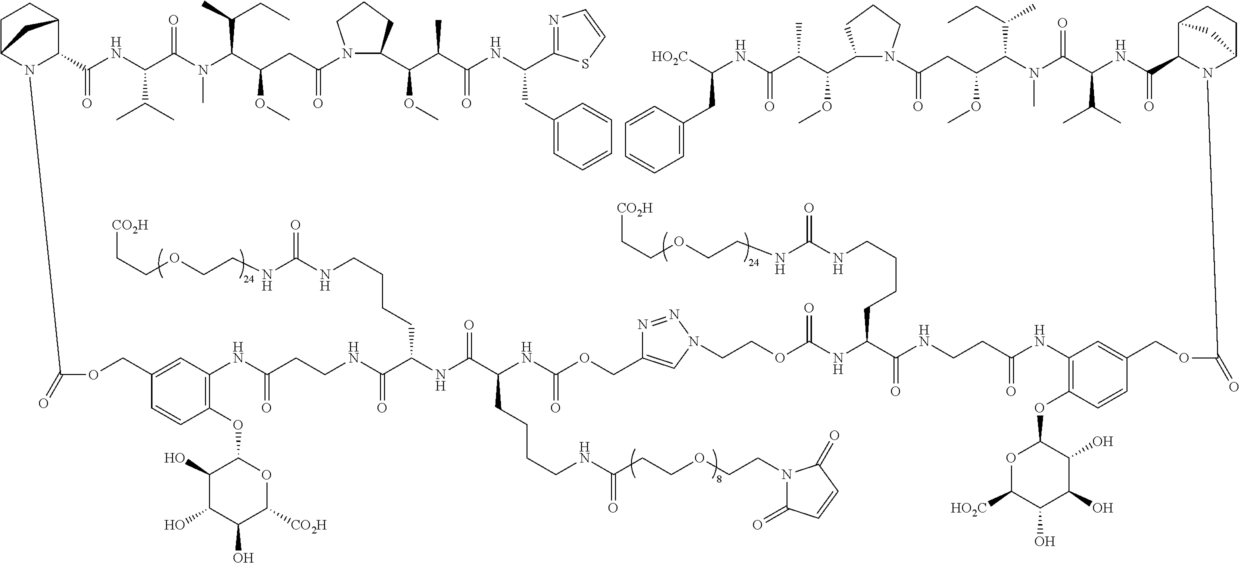 |
| P4- L27-P4 | 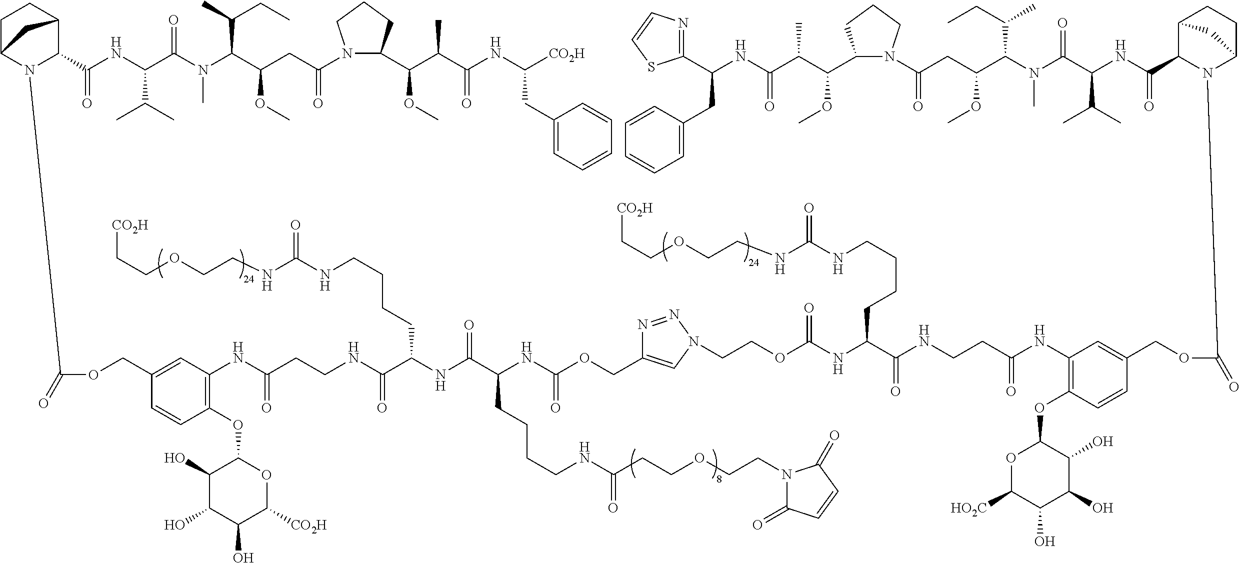 |
| P1- L28-P1 | 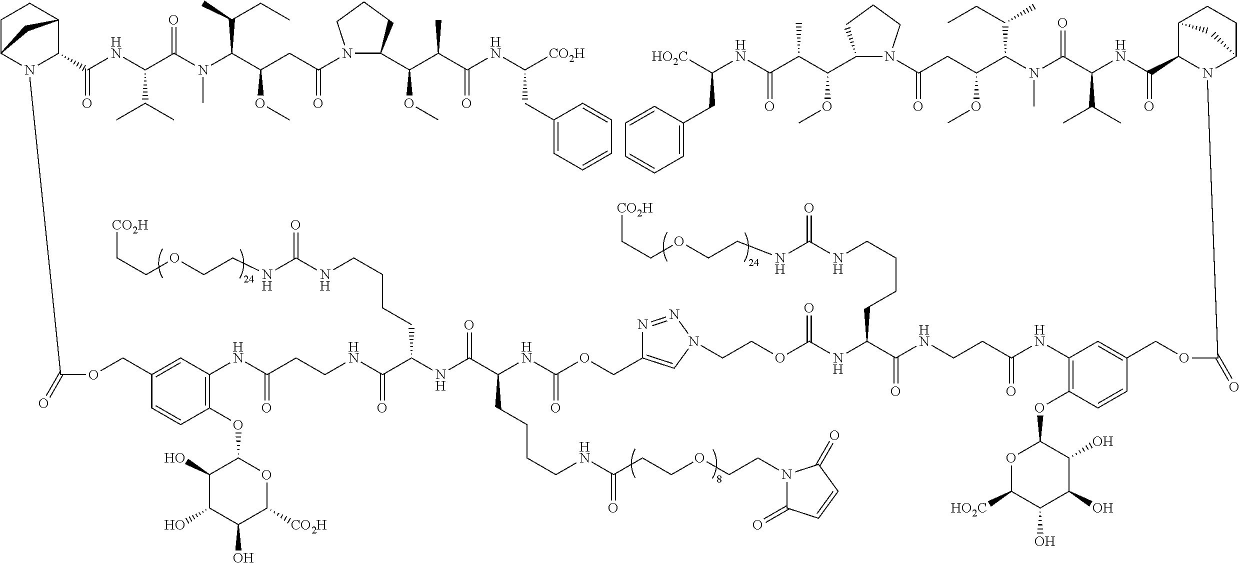 |
| P1- L28-P2 | 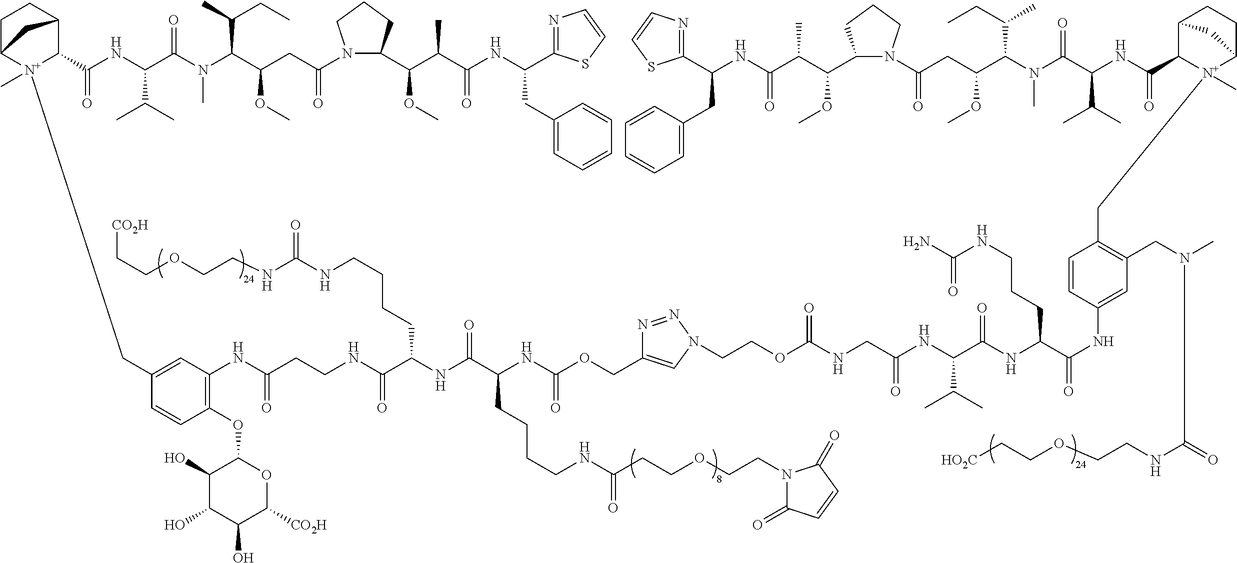 |
| P2- L28-P1 | 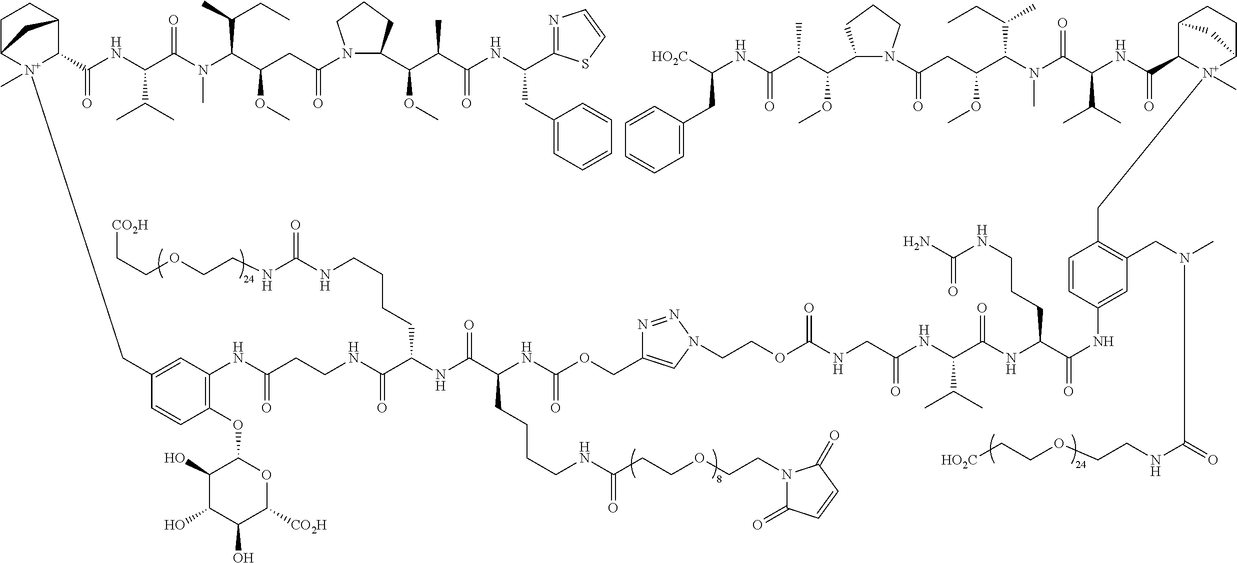 |
| P2- L28-P2 | 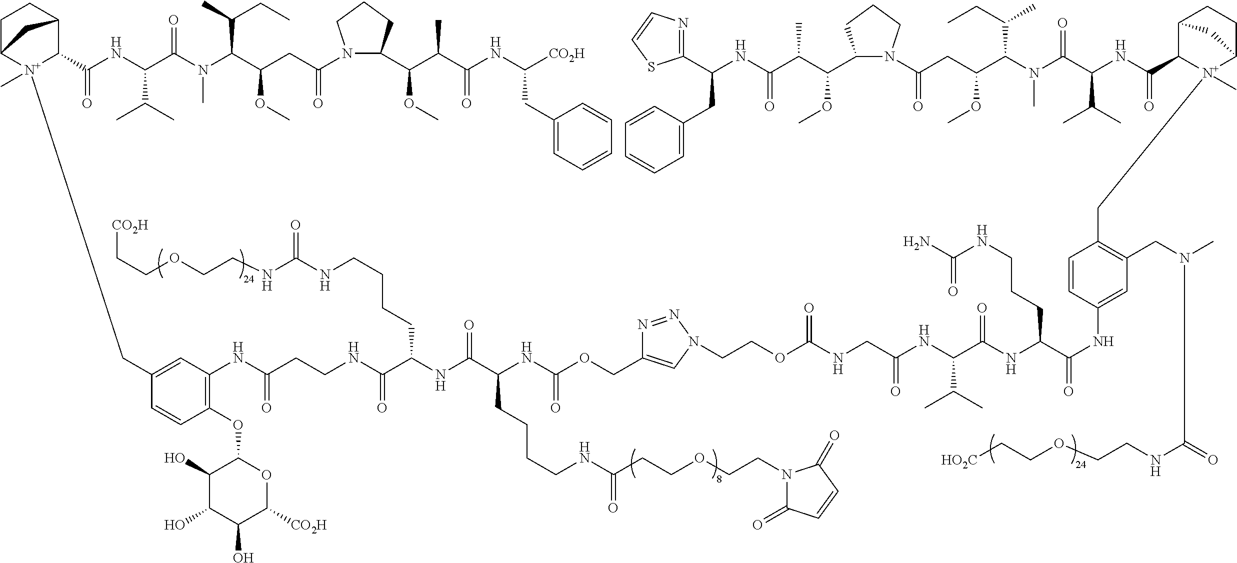 |
| P3- L28-P3 | 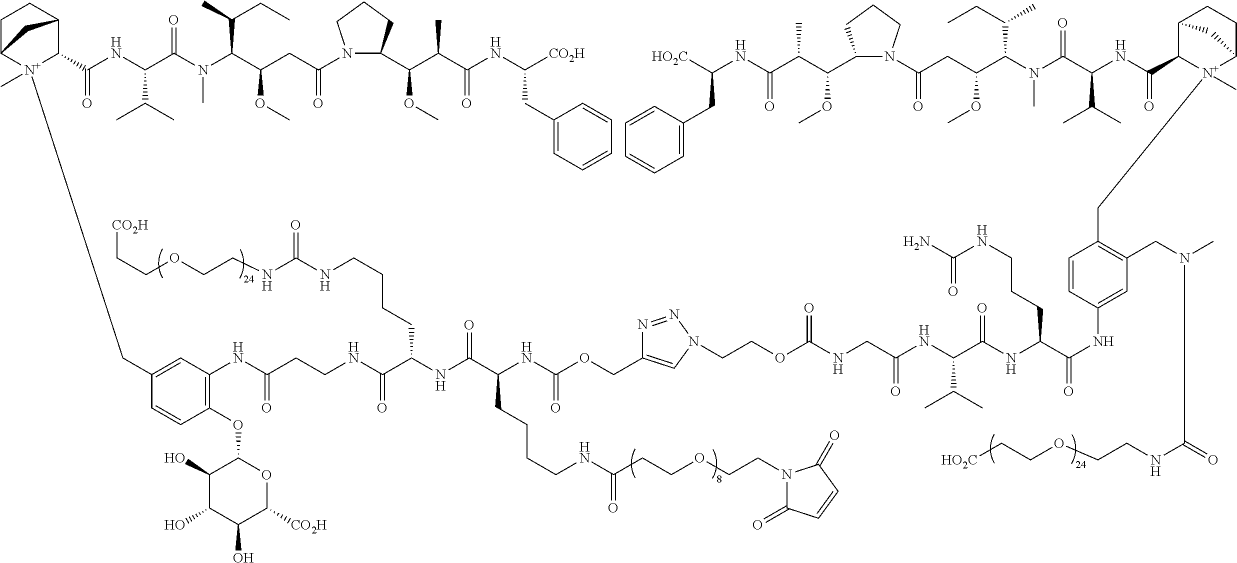 |
| P3- L28-P4 | 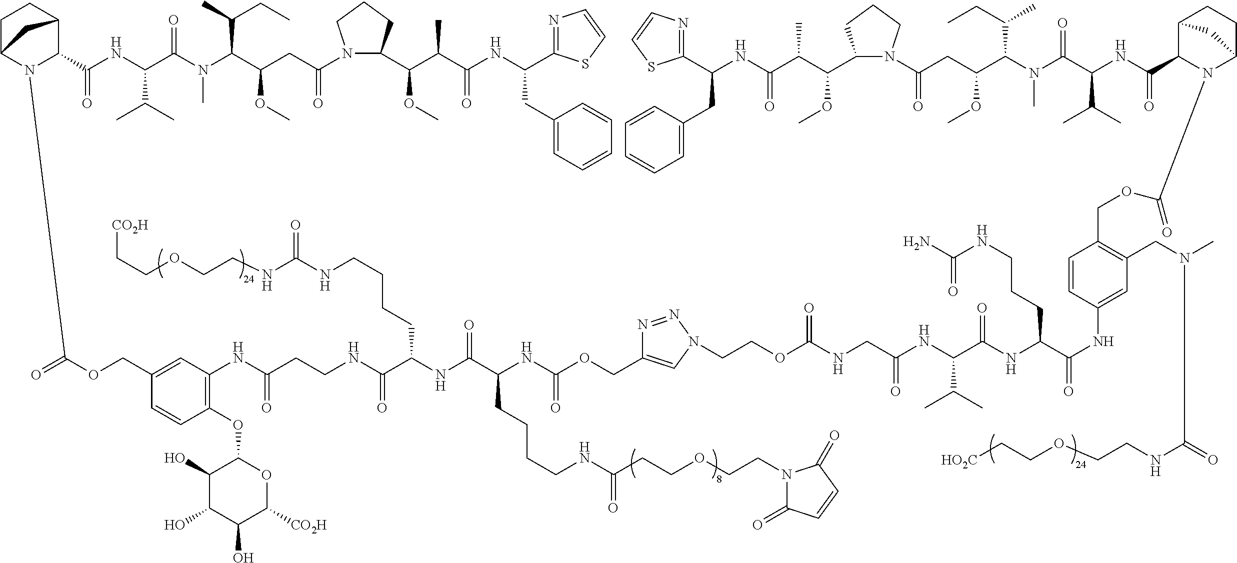 |
| P4- L28-P3 | 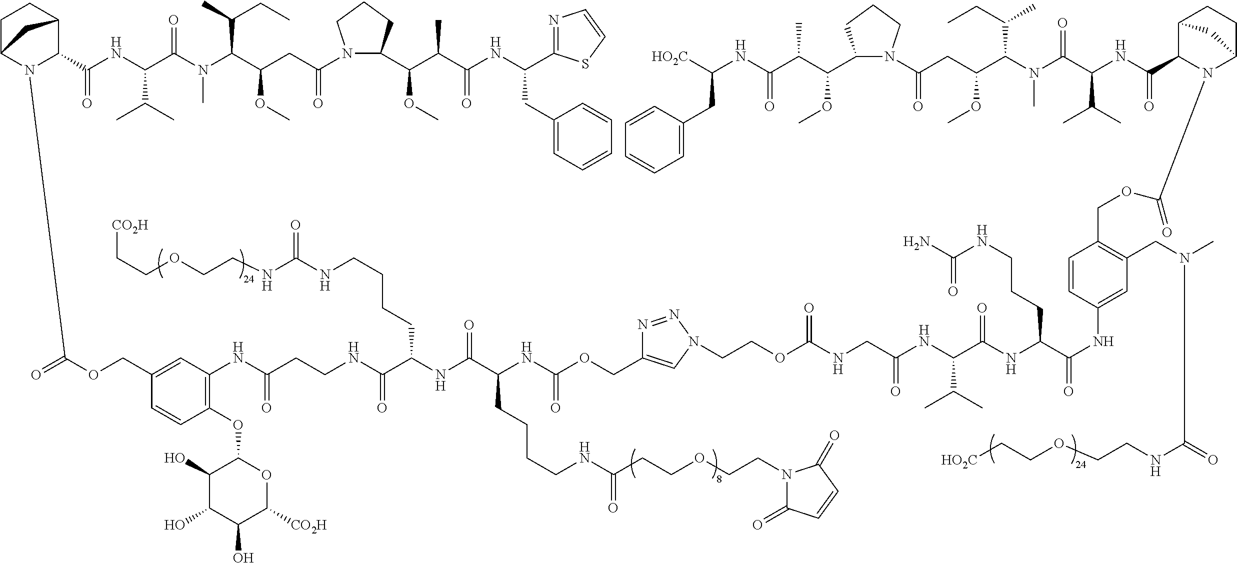 |
| P4- L28-P4 | 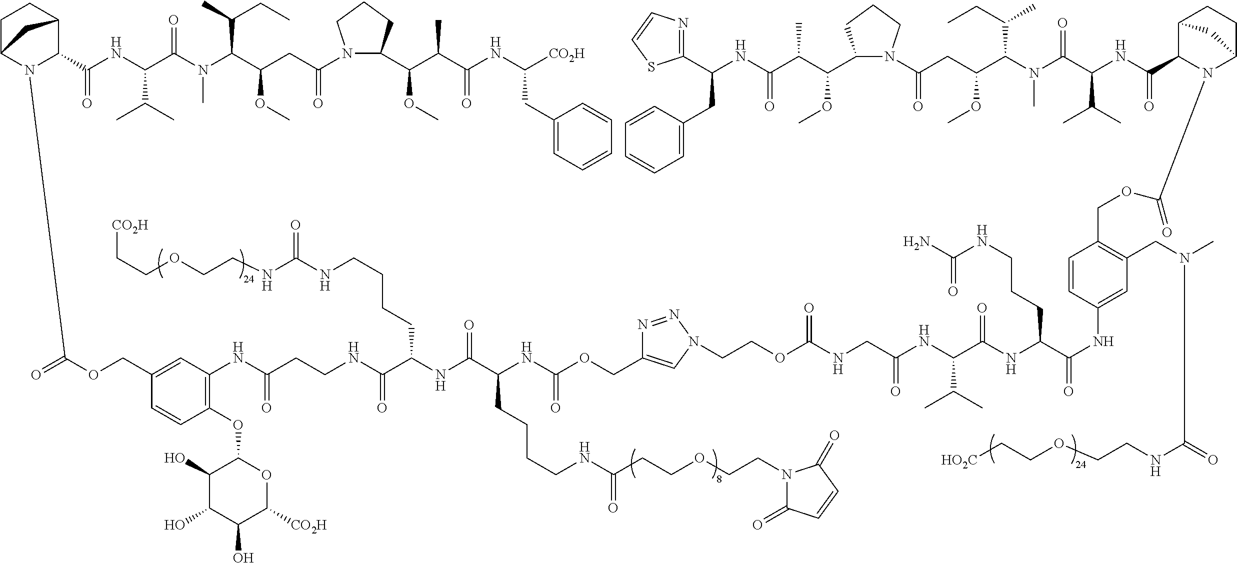 |
| P1- L29-P1 | 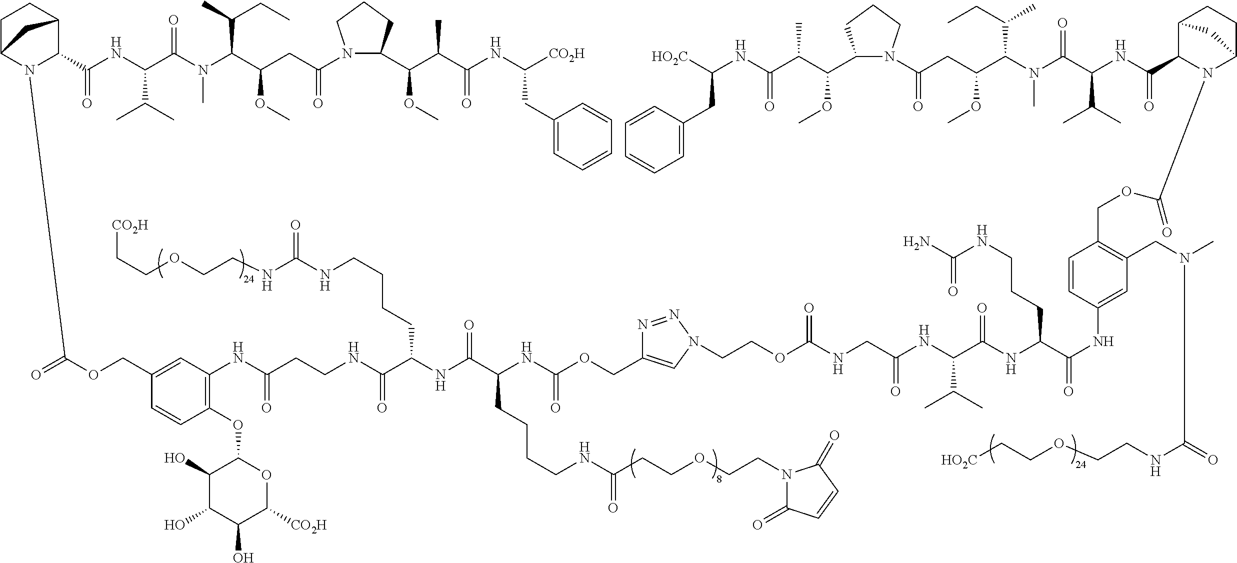 |
| P1- L29-P2 | 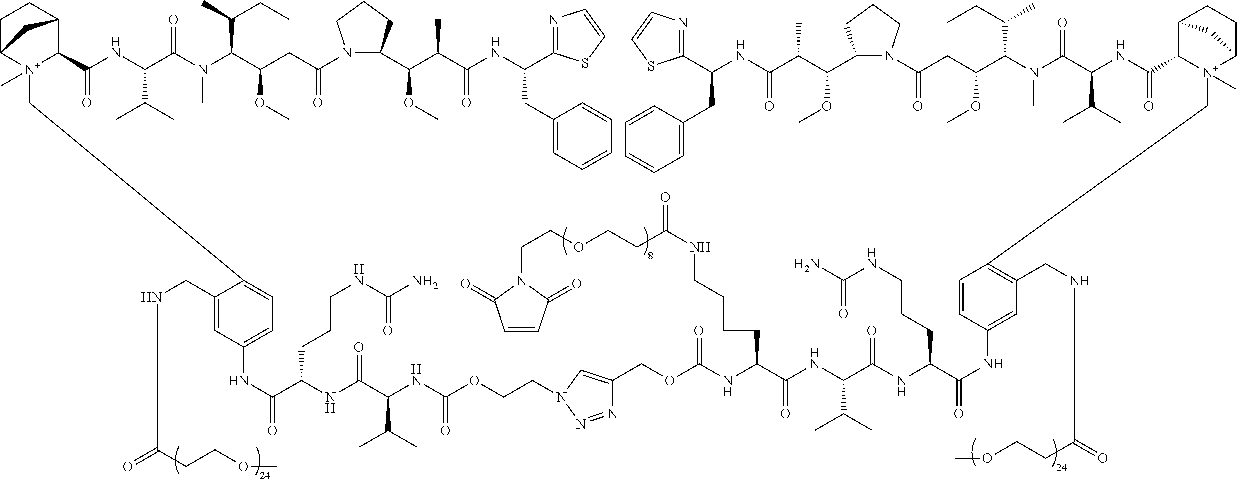 |
| P2- L29-P1 | 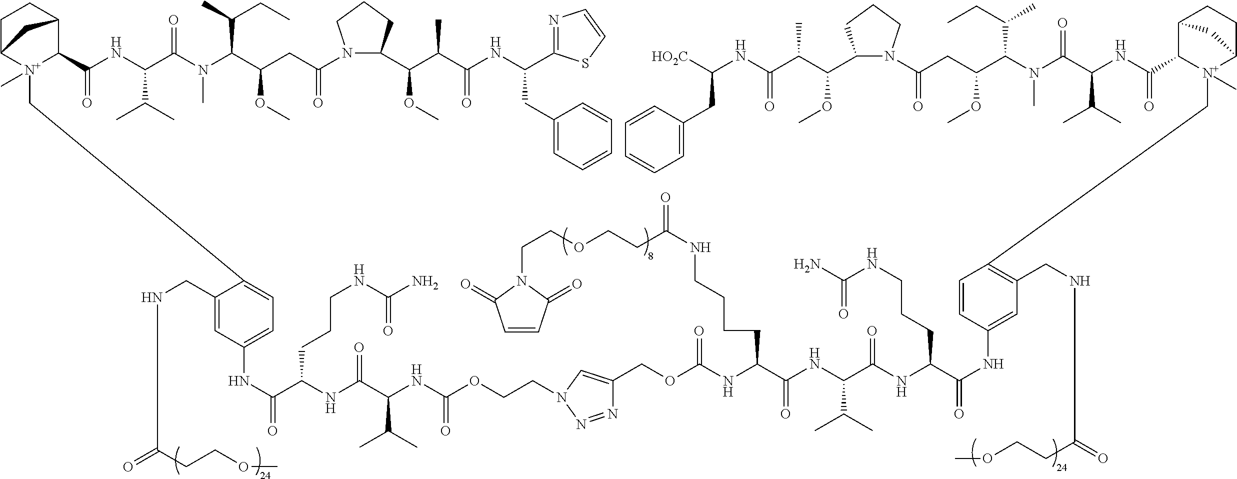 |
| P2- L29-P2 | 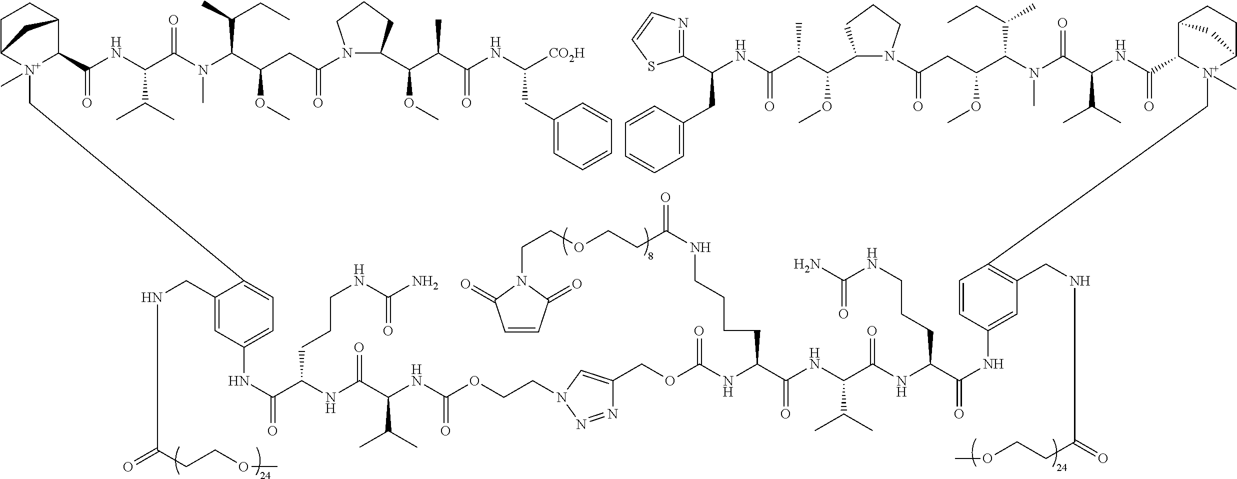 |
| P3- L29-P3 | 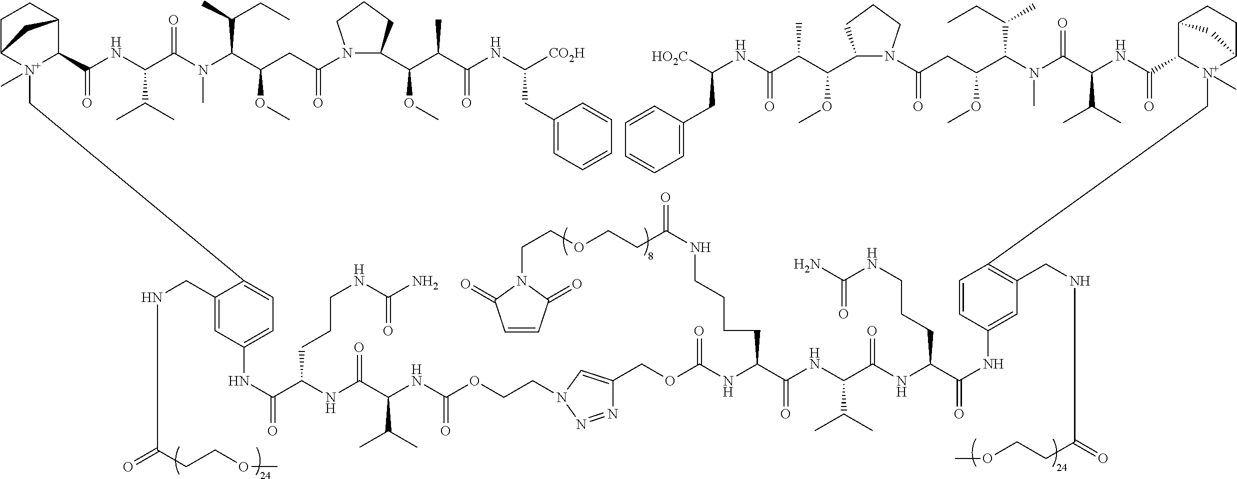 |
| P3- L29-P4 | 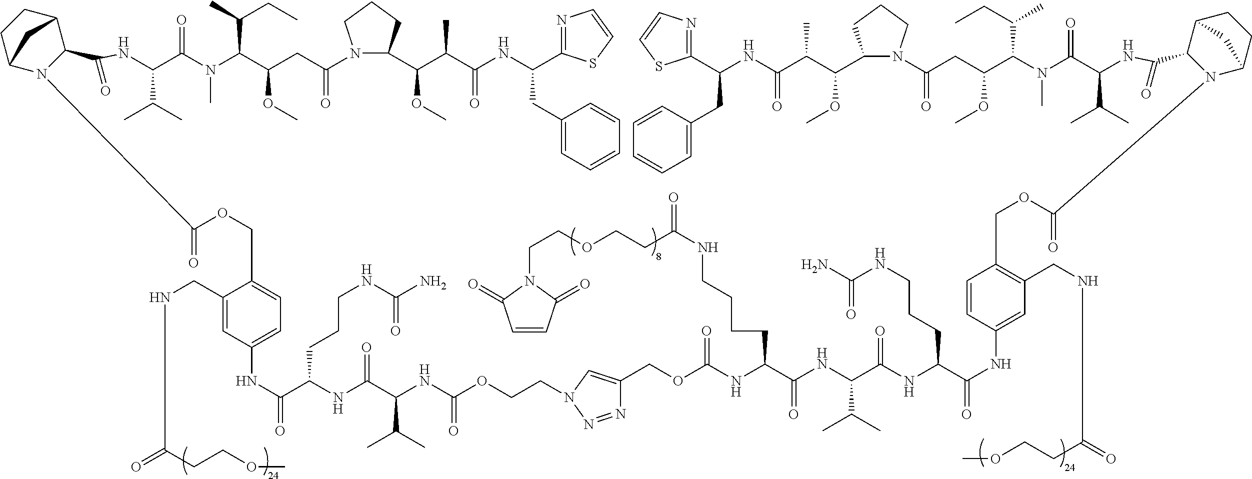 |
| P4- L29-P3 | 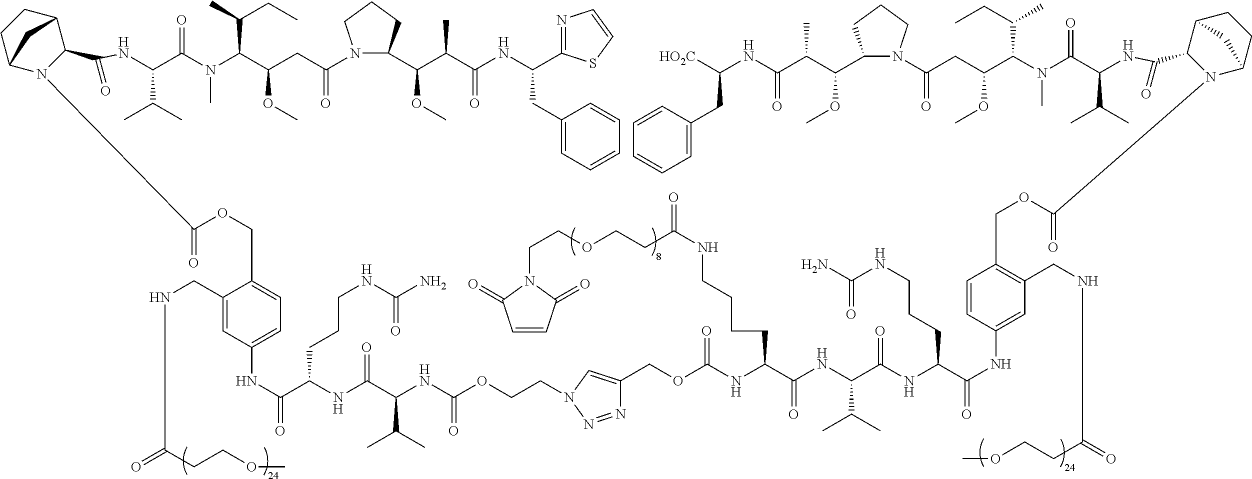 |
| P4- L29-P4 | 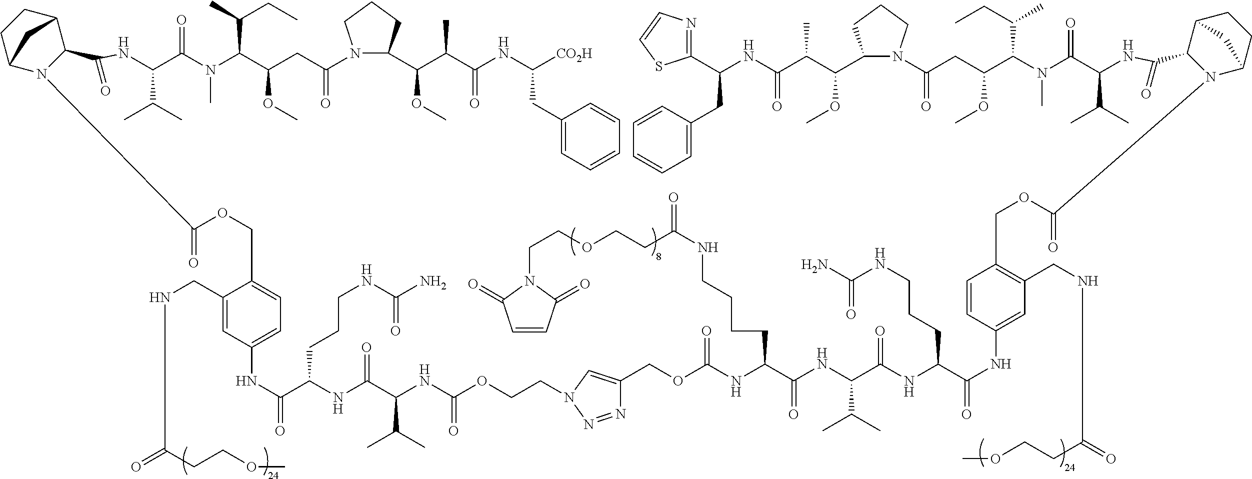 |
| P1- L30-P1 | 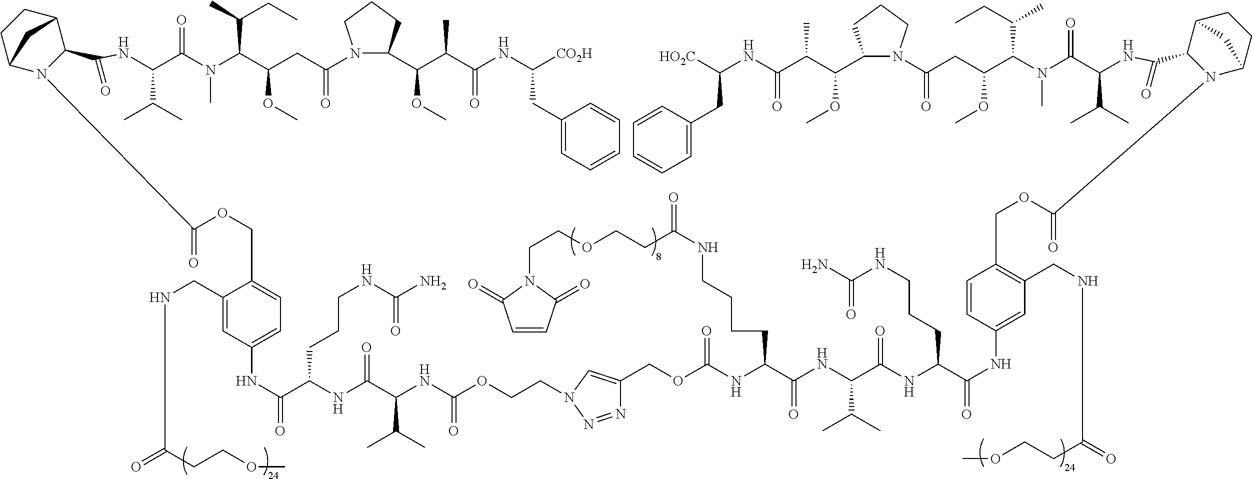 |
| P1- L30-P2 | 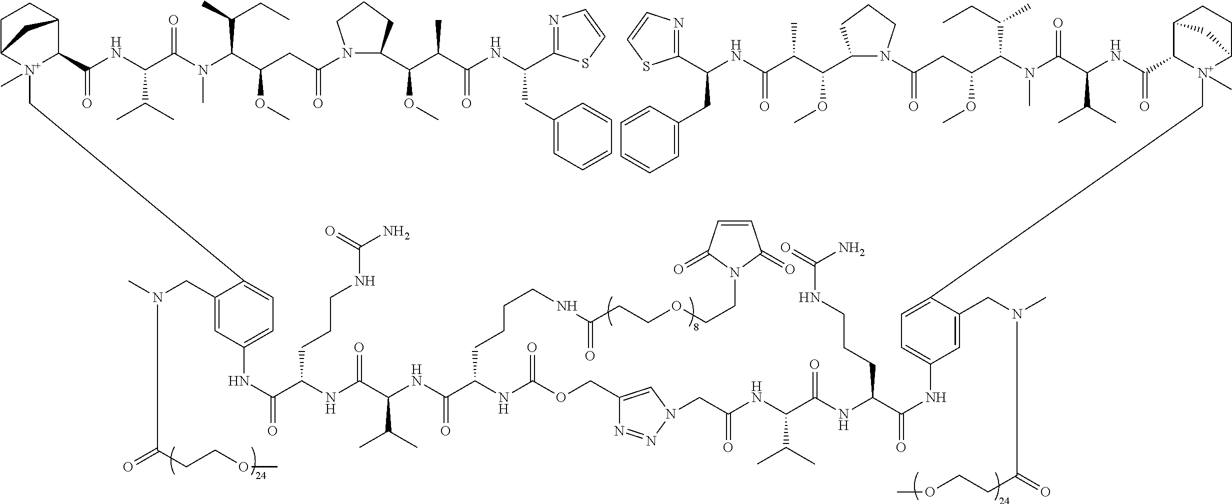 |
| P2- L30-P1 | 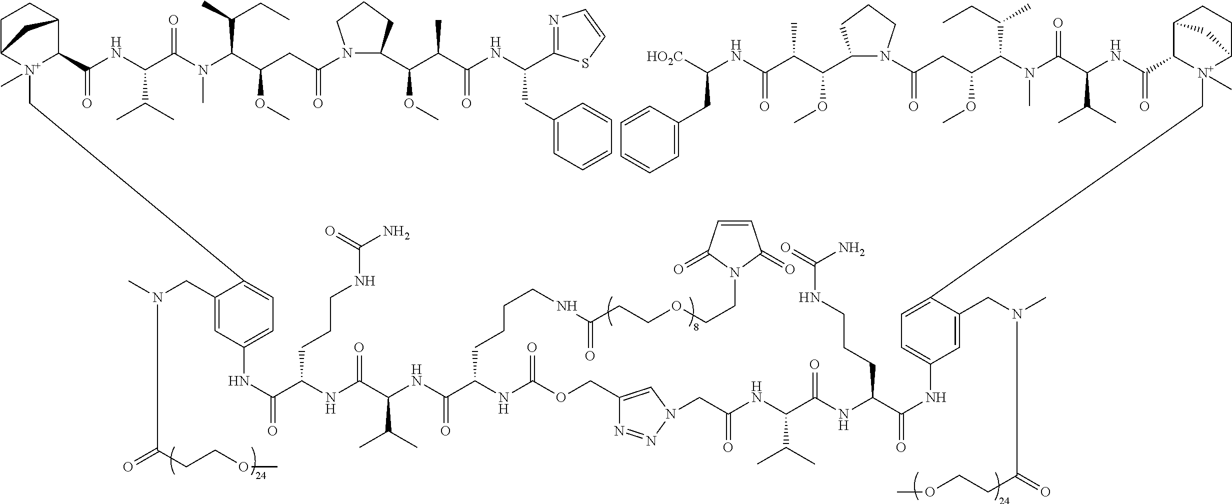 |
| P2- L30-P2 | 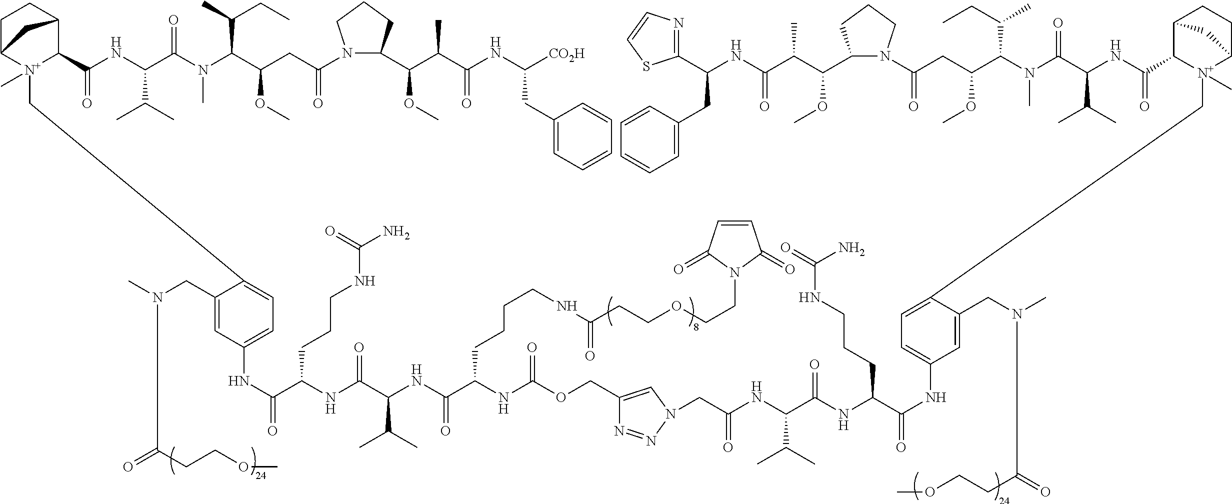 |
| P3- L30-P3 | 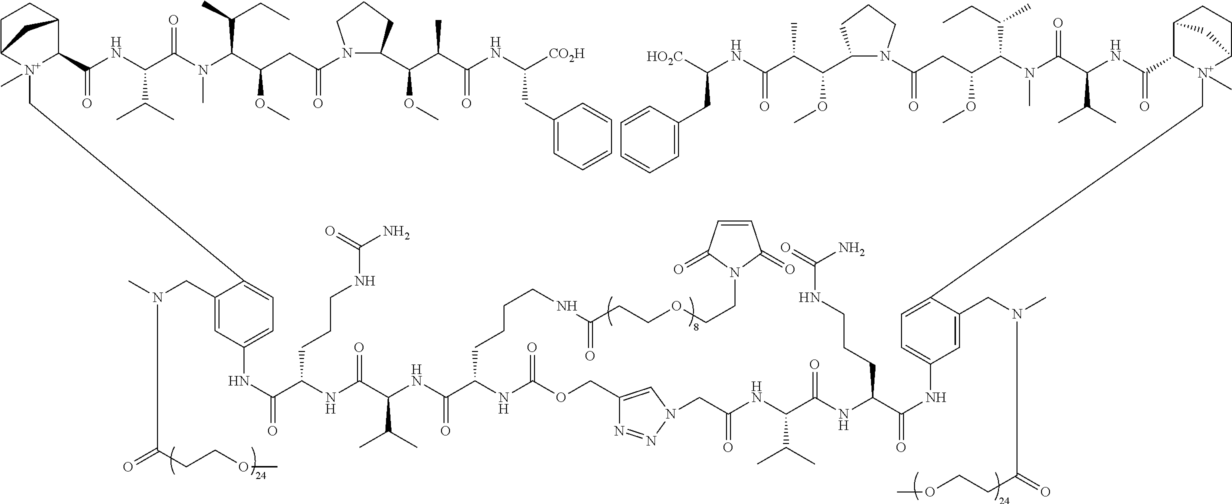 |
| P3- L30-P4 | 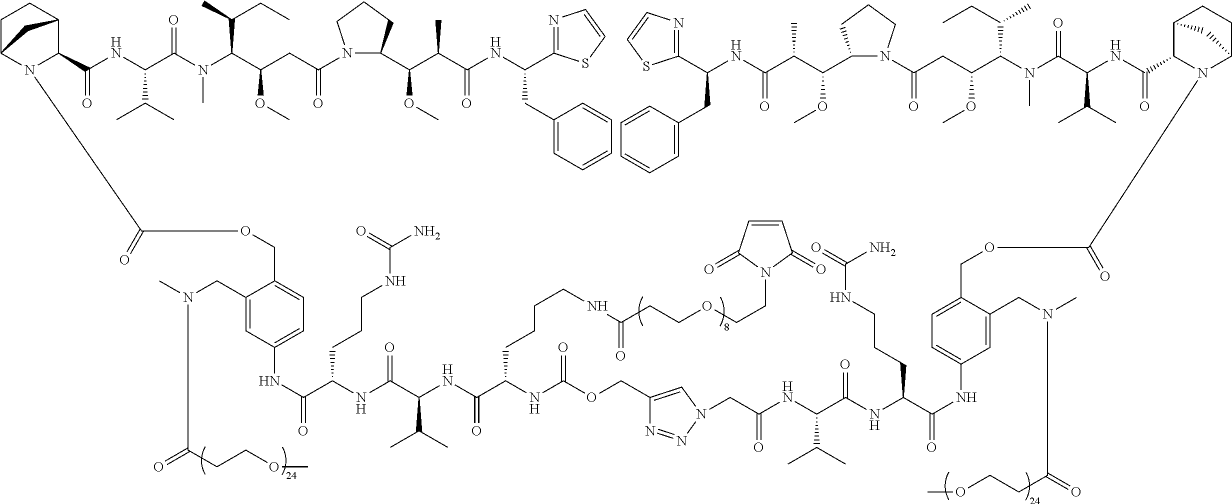 |
| P4- L30-P3 | 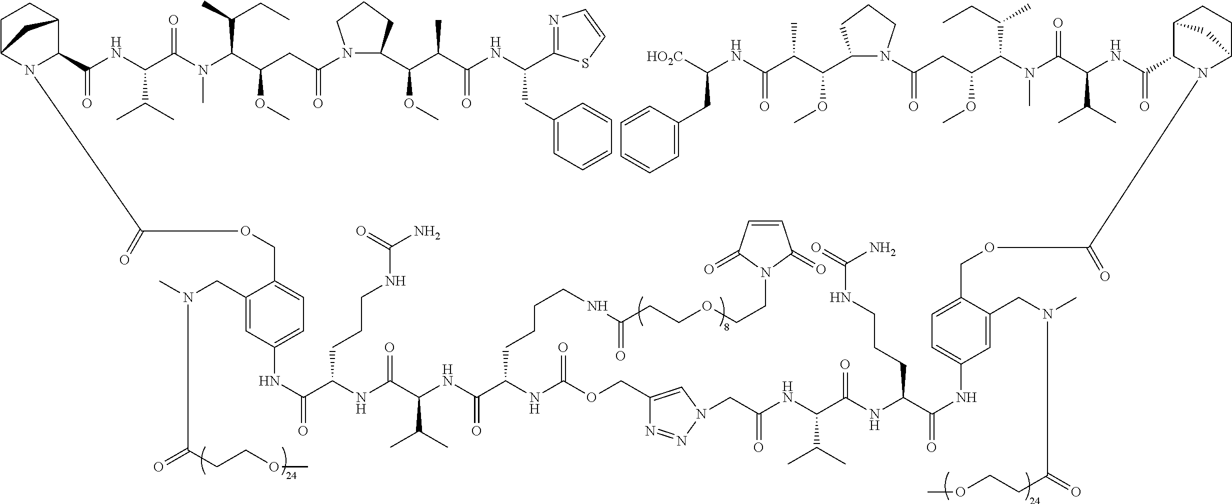 |
| P4- L30-P4 | 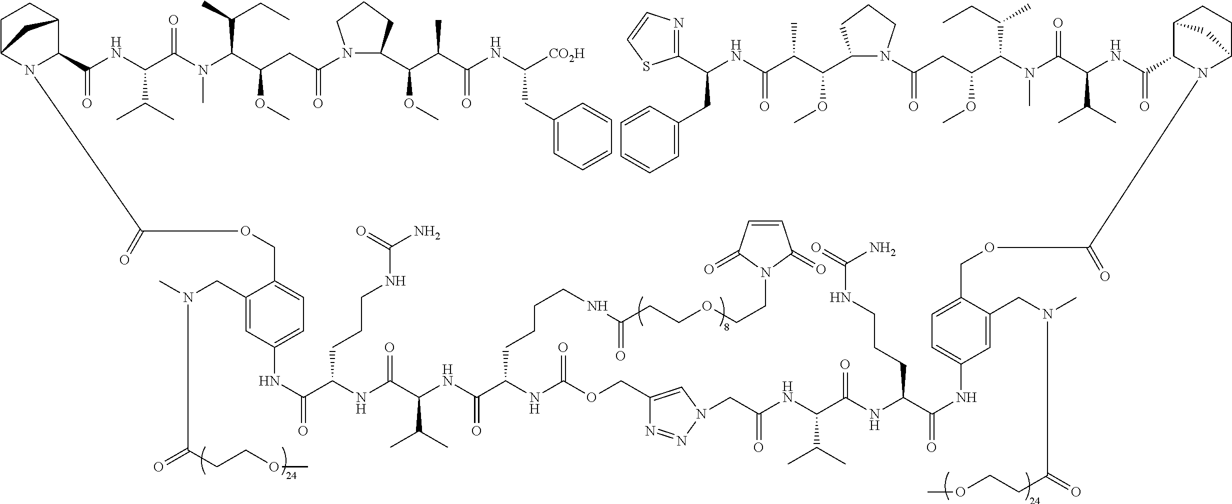 |
| P1- L31-P1 | 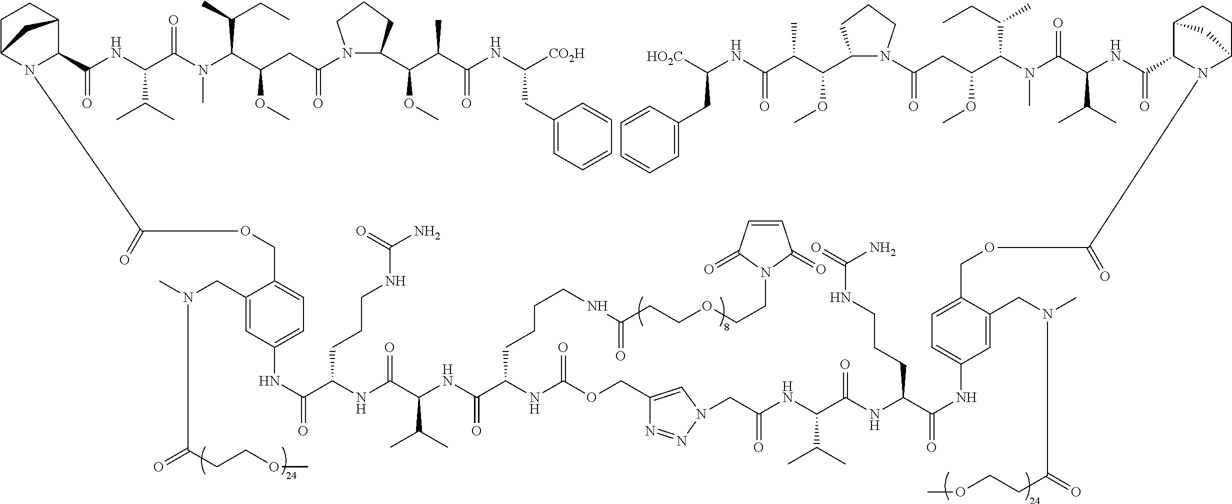 |
| P1- L31-P2 | 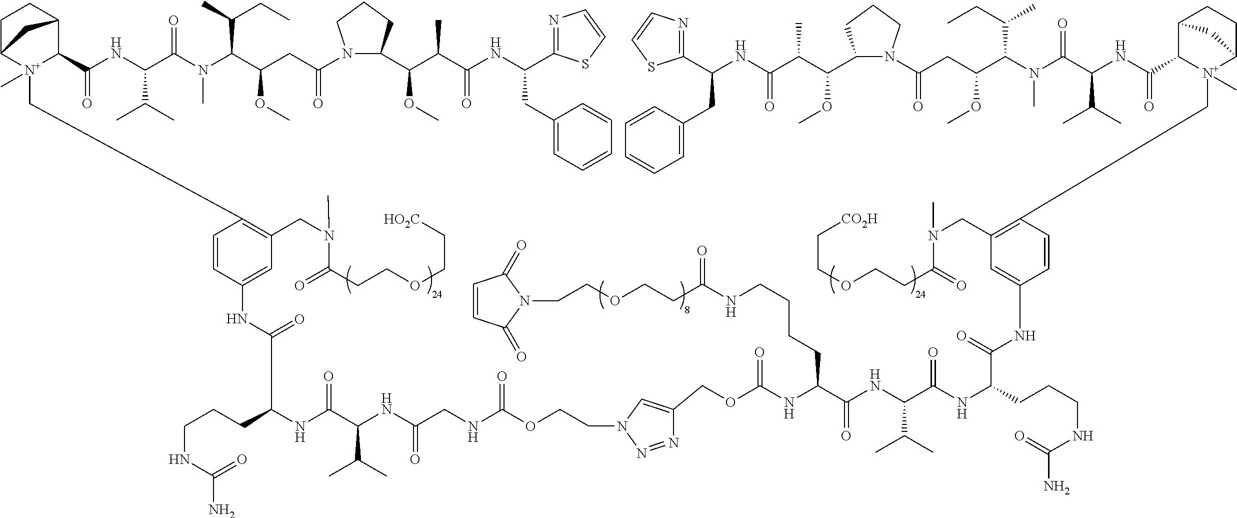 |
| P2- L31-P1 | 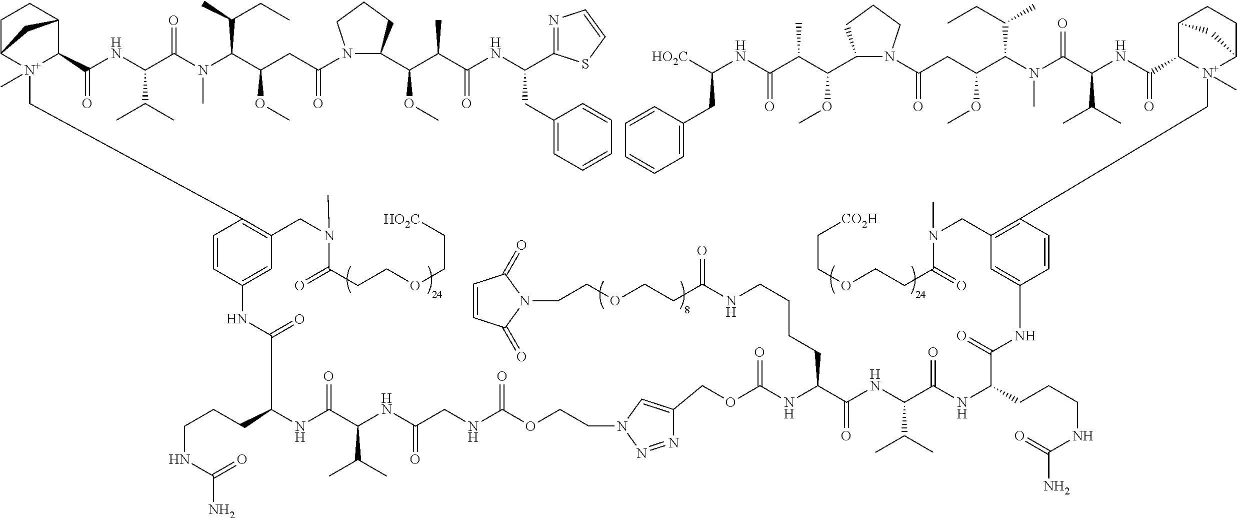 |
| P2- L31-P2 | 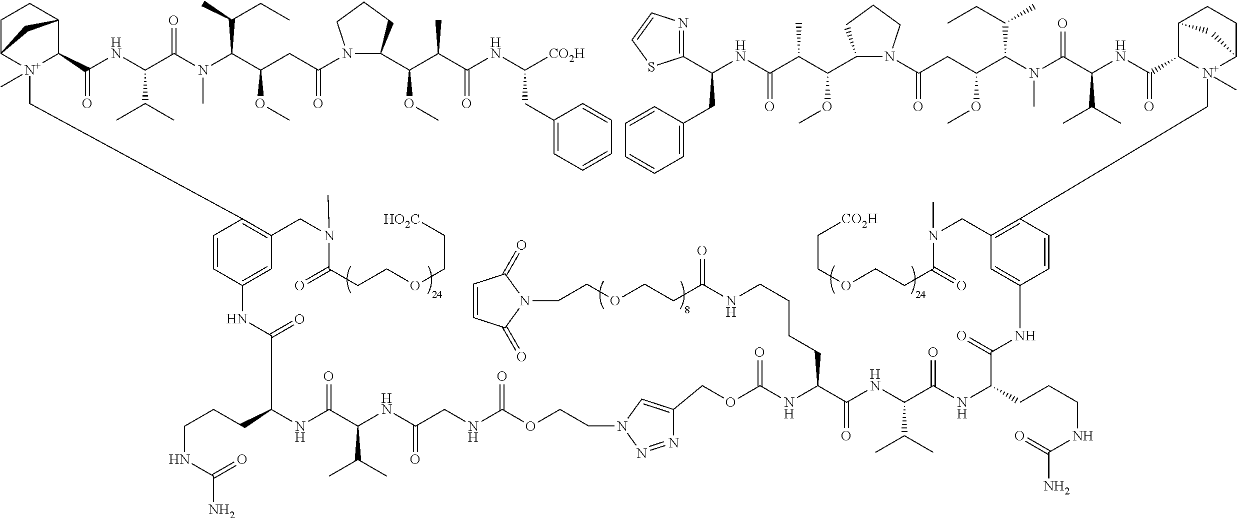 |
| P3- L31-P3 | 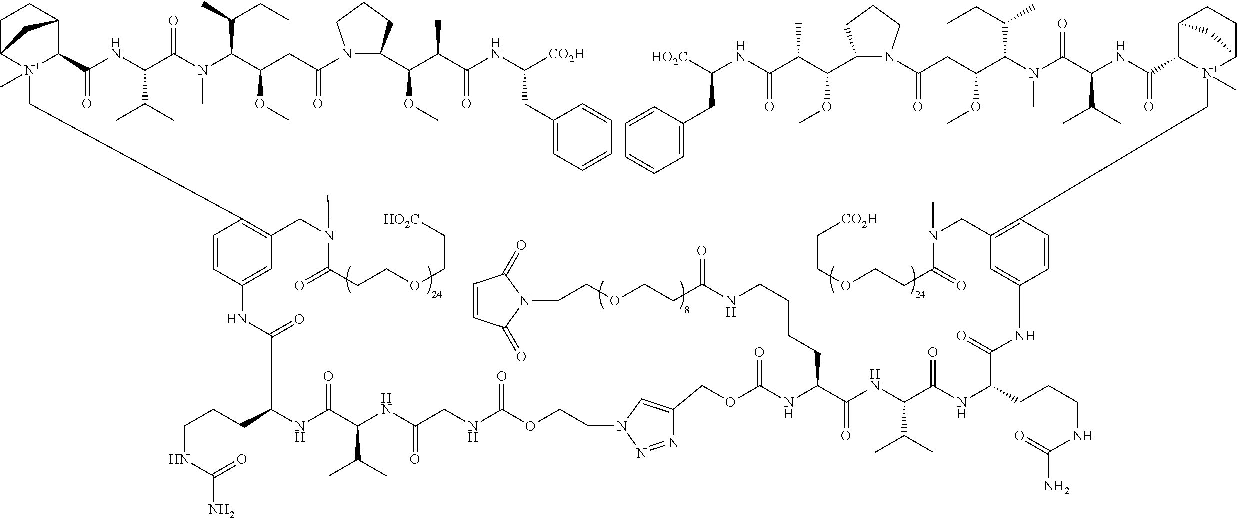 |
| P3- L31-P4 | 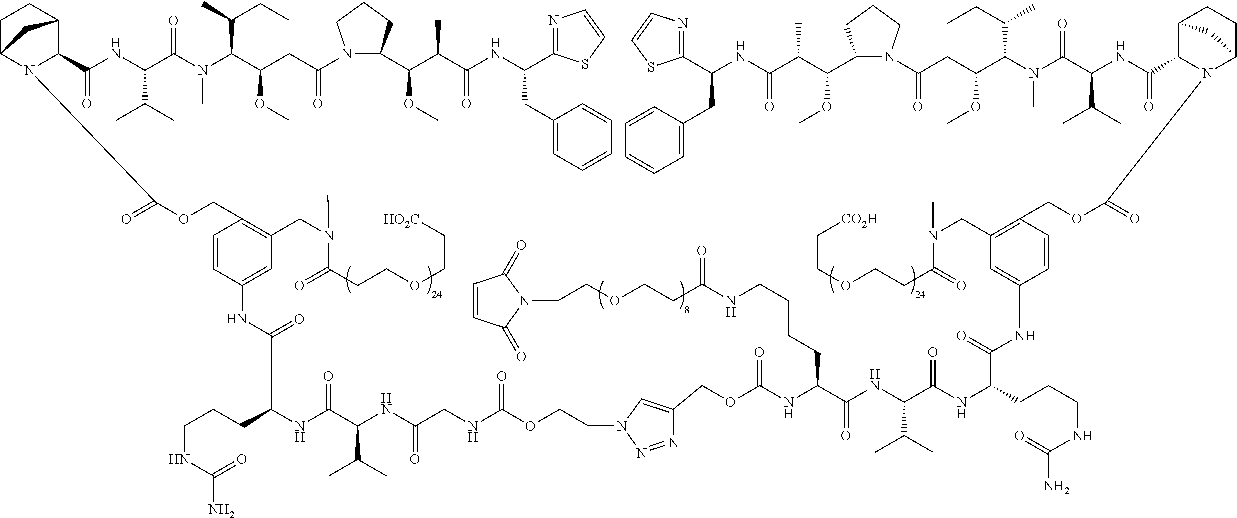 |
| P4- L31-P3 | 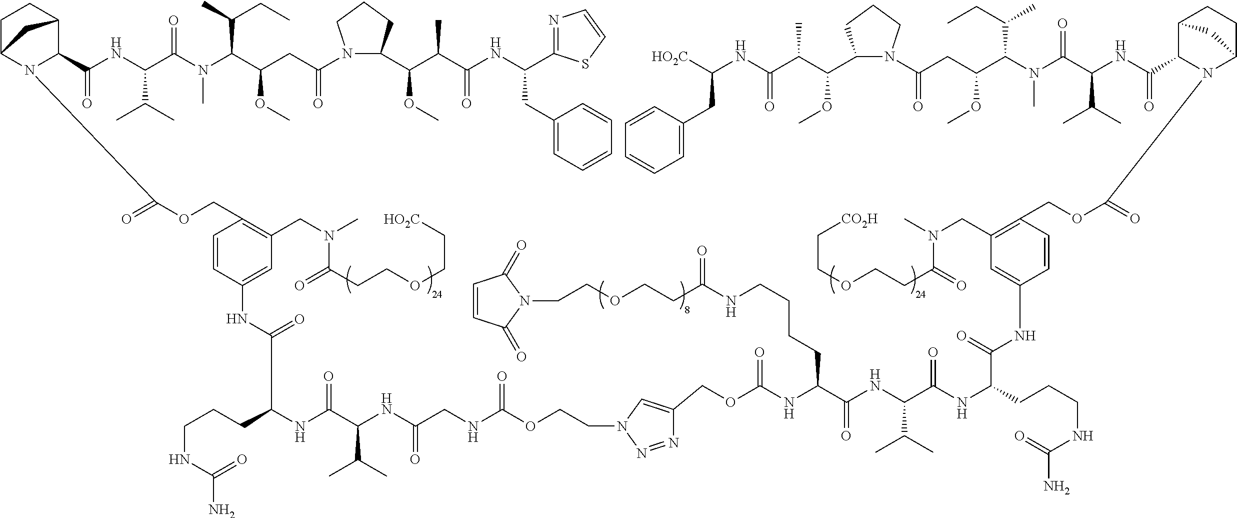 |
| P4- L31-P4 | 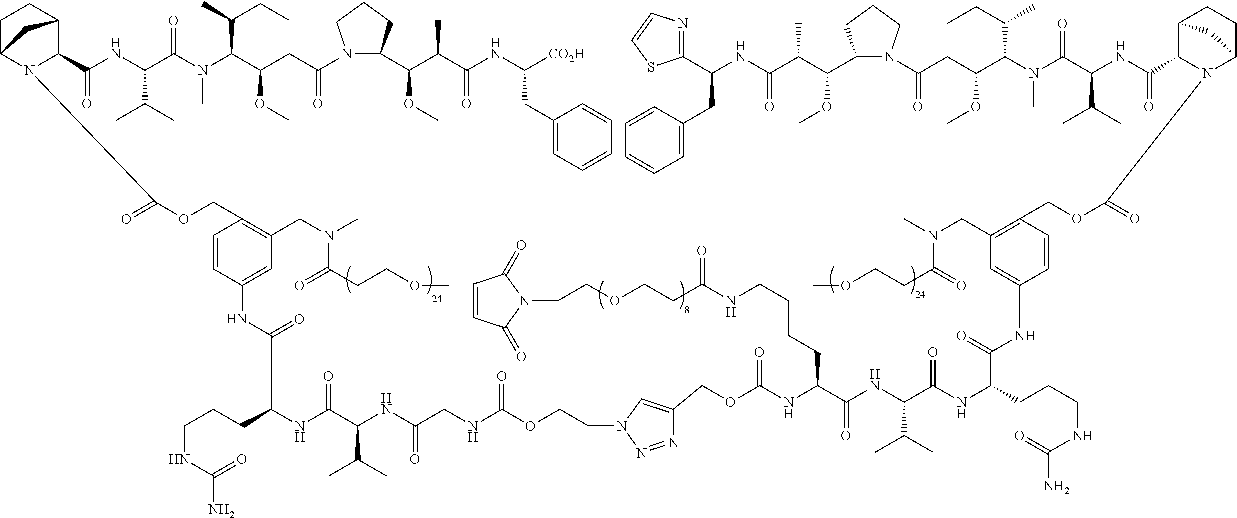 |
| P1- L32-P1 | 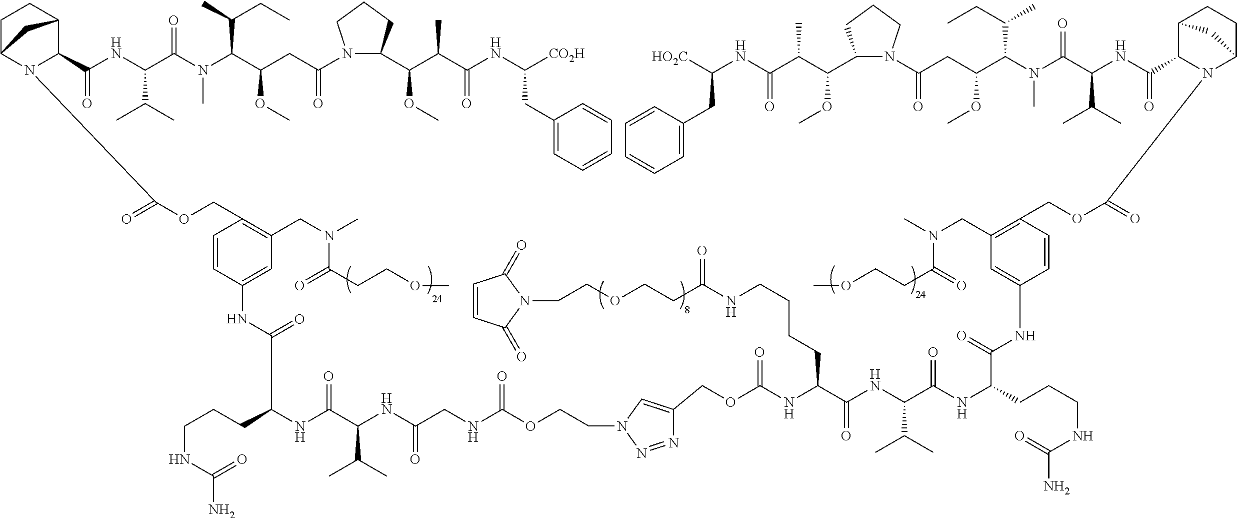 |
| P1- L32-P2 | 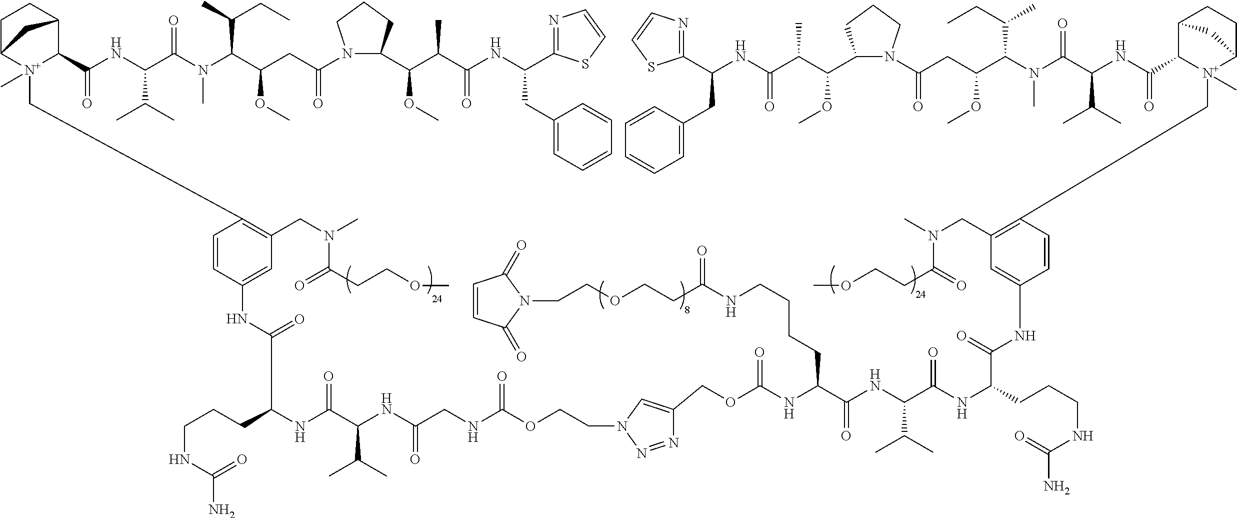 |
| P2- L32-P1 | 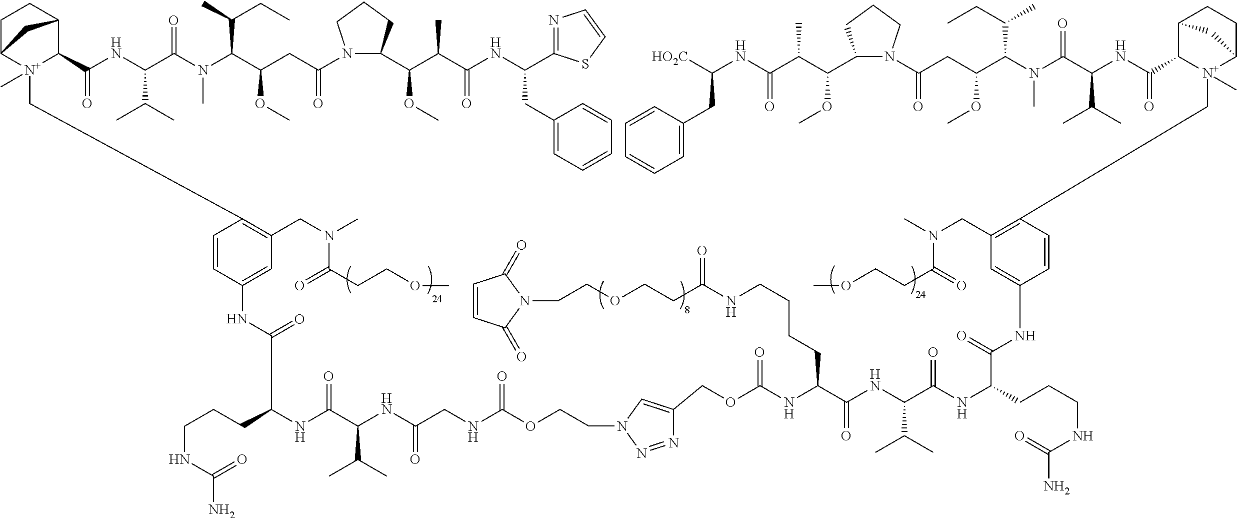 |
| P2- L32-P2 | 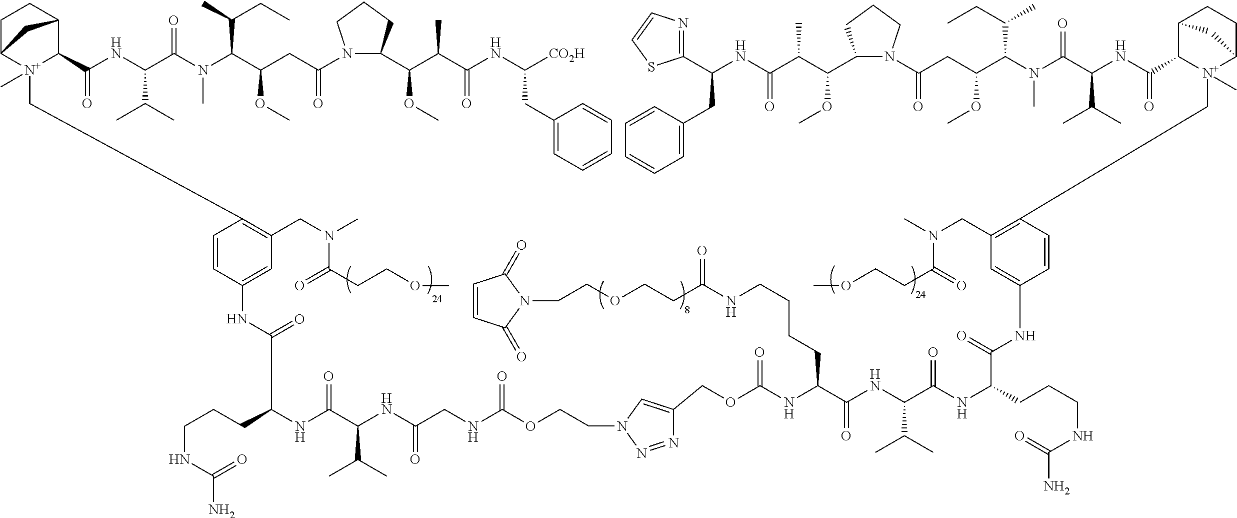 |
| P3- L32-P3 | 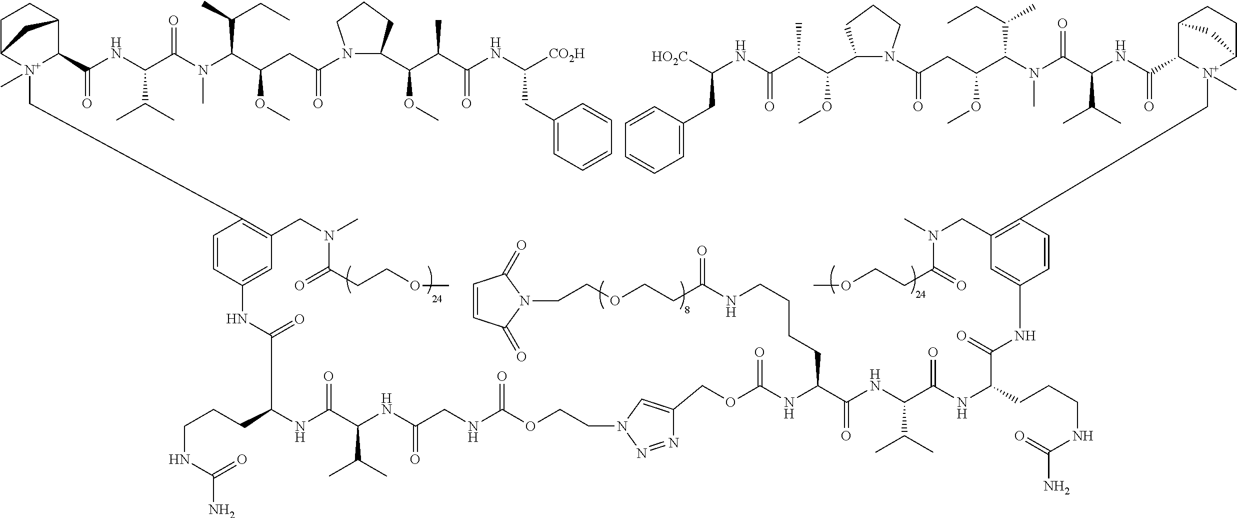 |
| P3- L32-P4 | 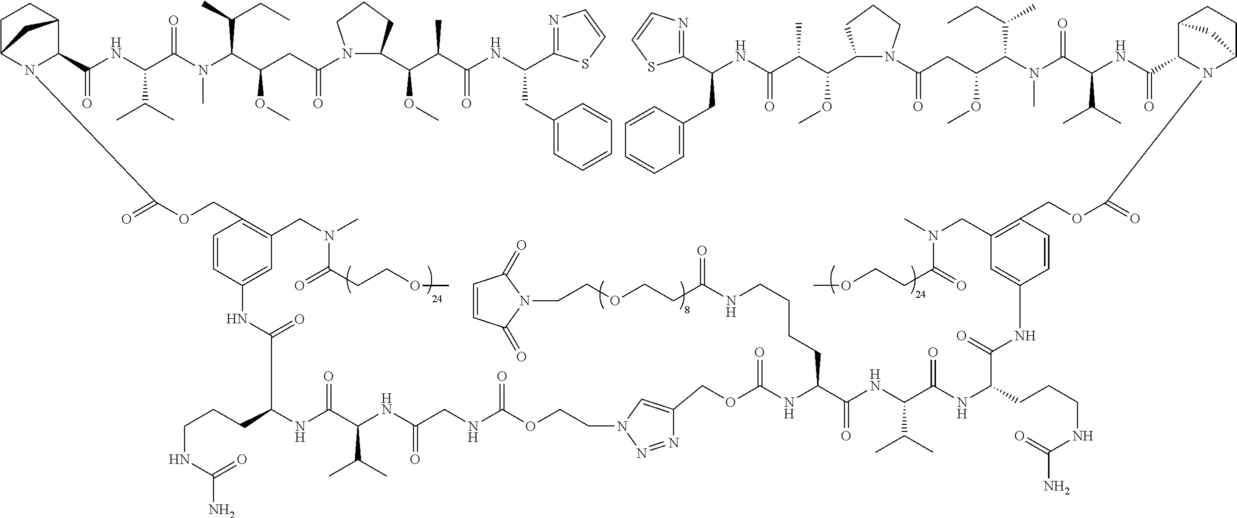 |
| P4- L32-P3 | 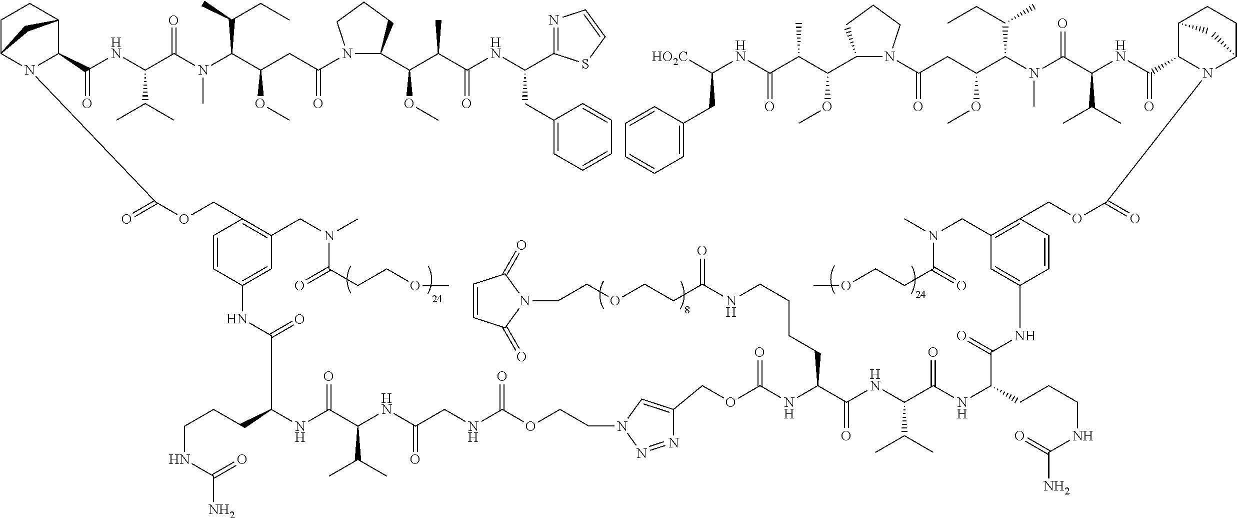 |
| P4- L32-P4 | 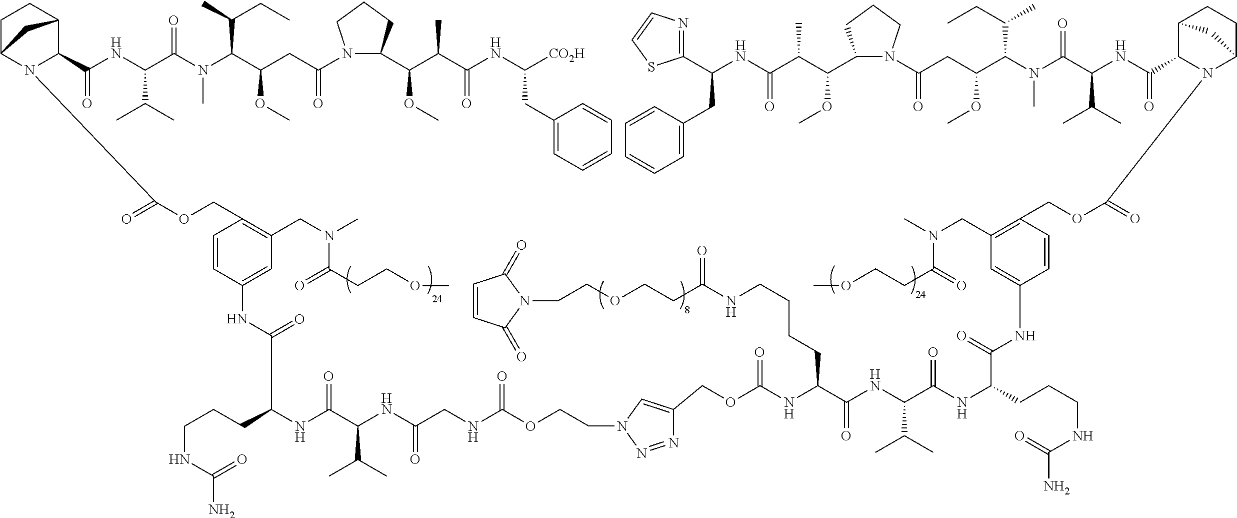 |
| P1- L33-P1 | 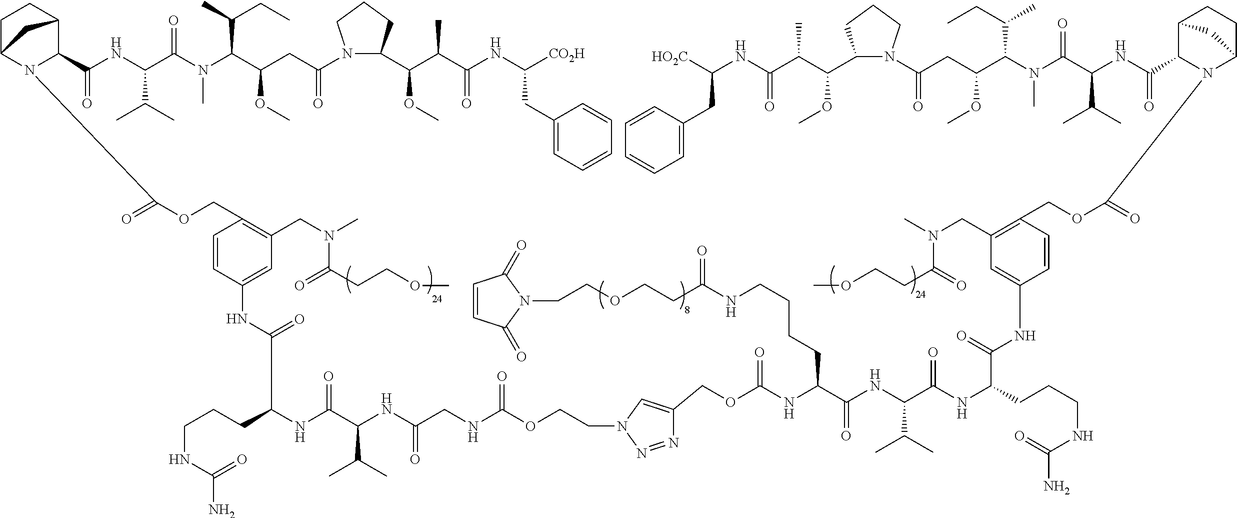 |
| P1- L33-P2 | 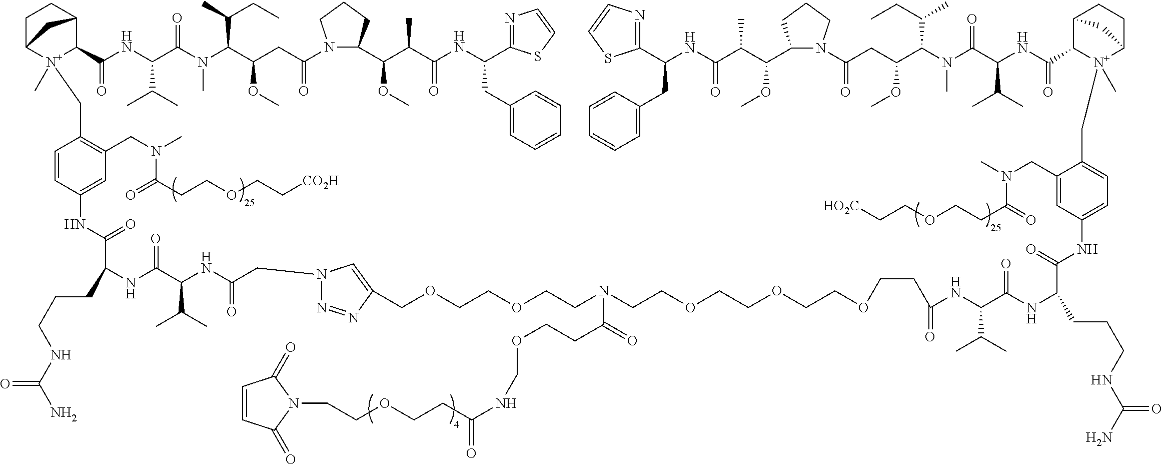 |
| P2- L33-P1 | 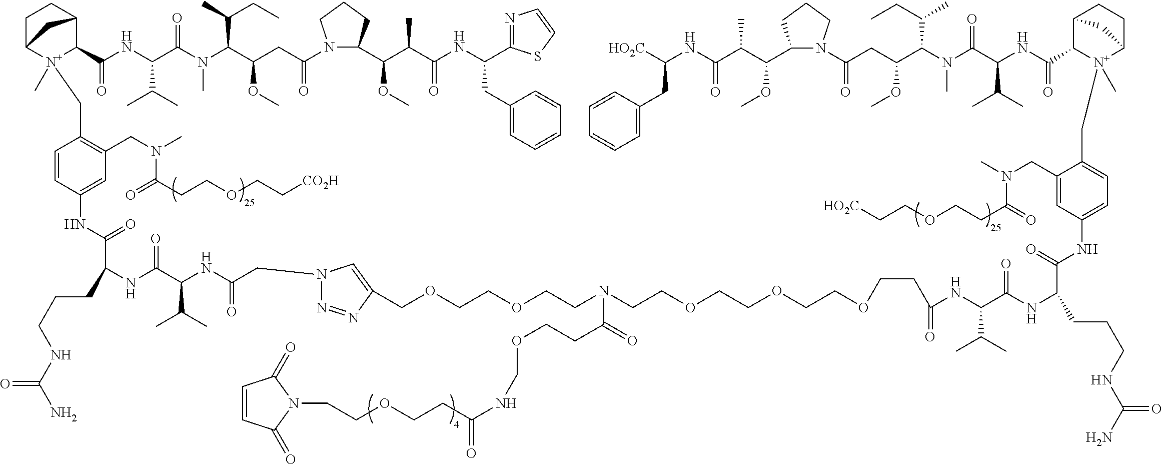 |
| P2- L33-P2 | 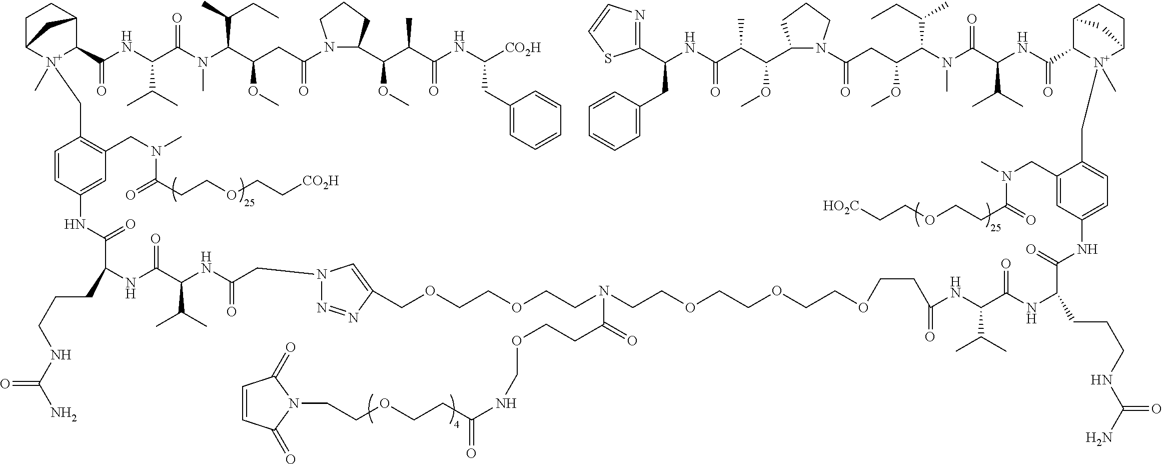 |
| P3- L33-P3 | 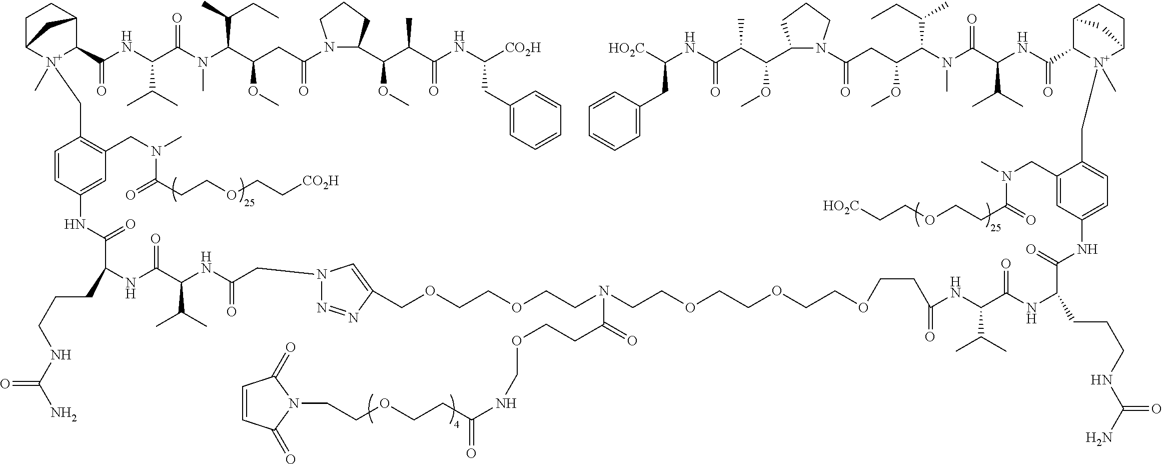 |
| P3- L33-P4 | 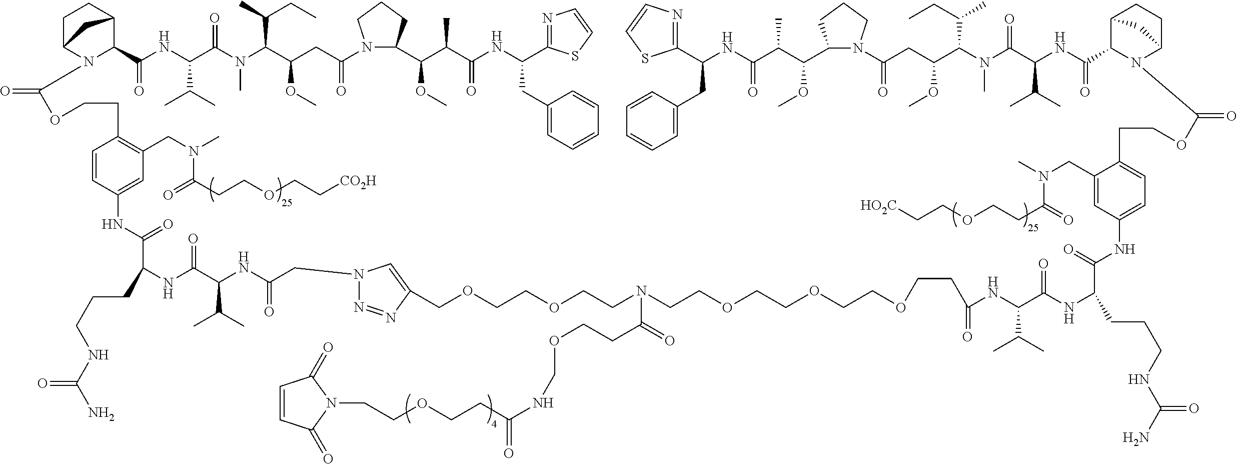 |
| P4- L33-P3 | 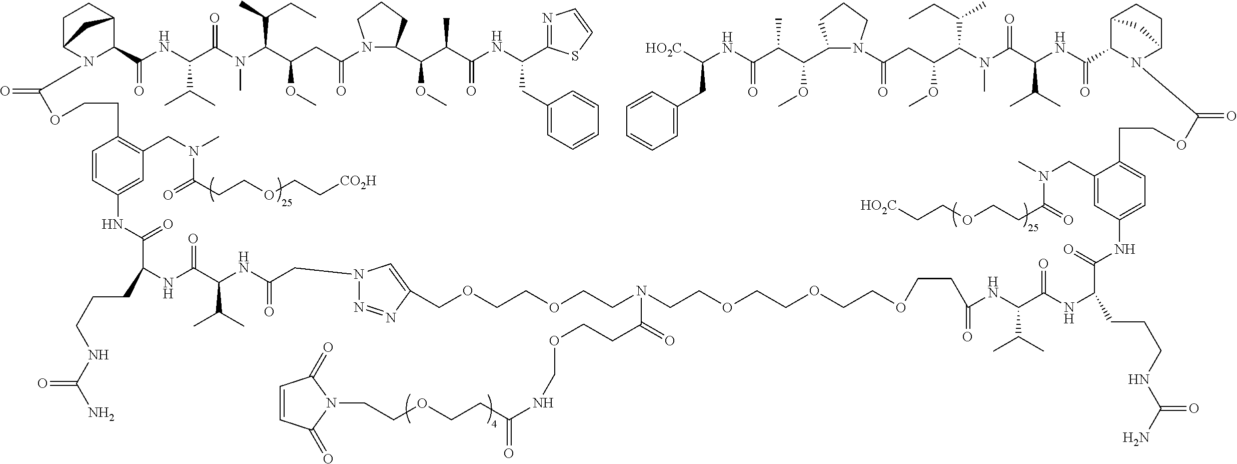 |
| P4- L33-P4 | 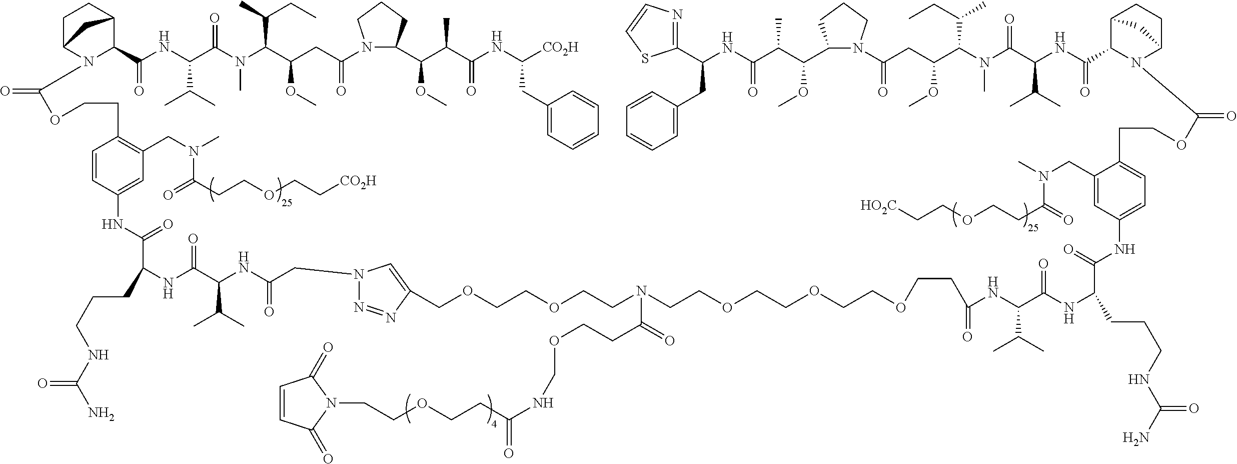 |
| P1- L34-P1 | 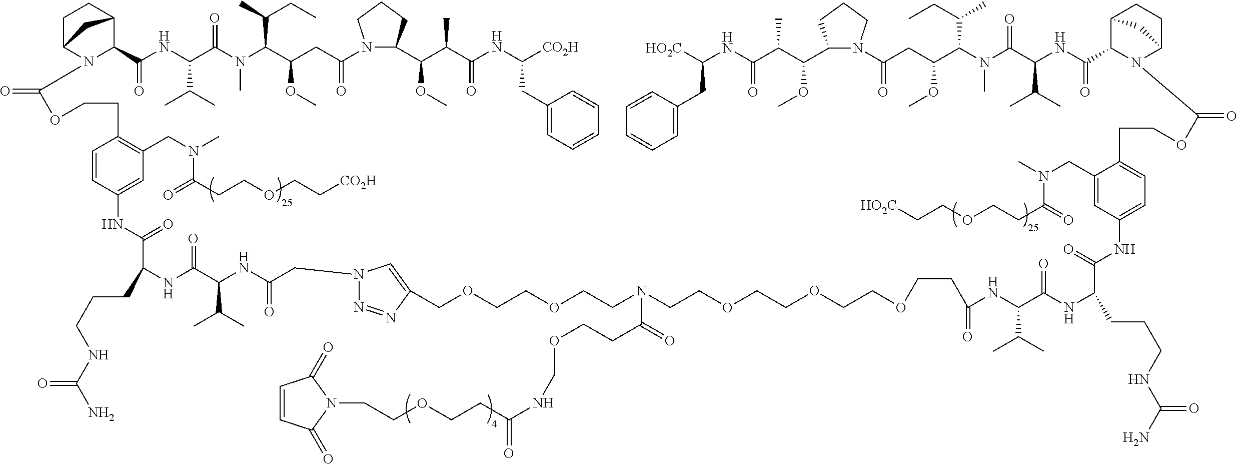 |
| P1- L34-P2 | 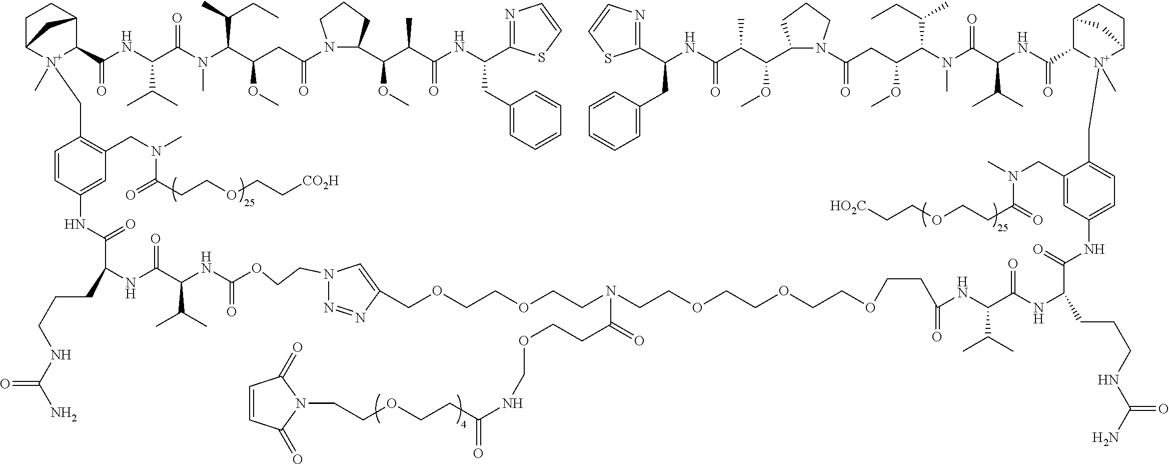 |
| P2- L34-P1 | 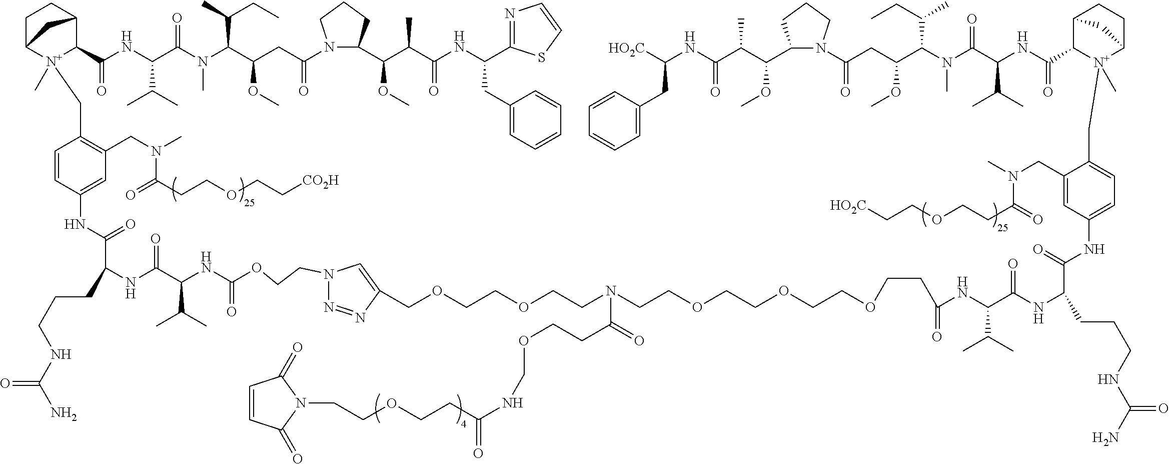 |
| P2- L34-P2 | 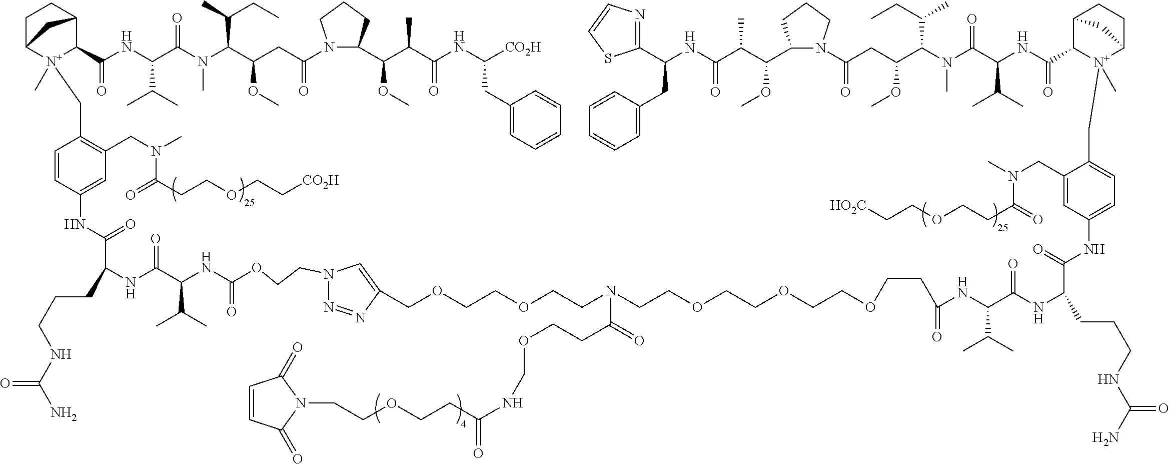 |
| P3- L34-P3 | 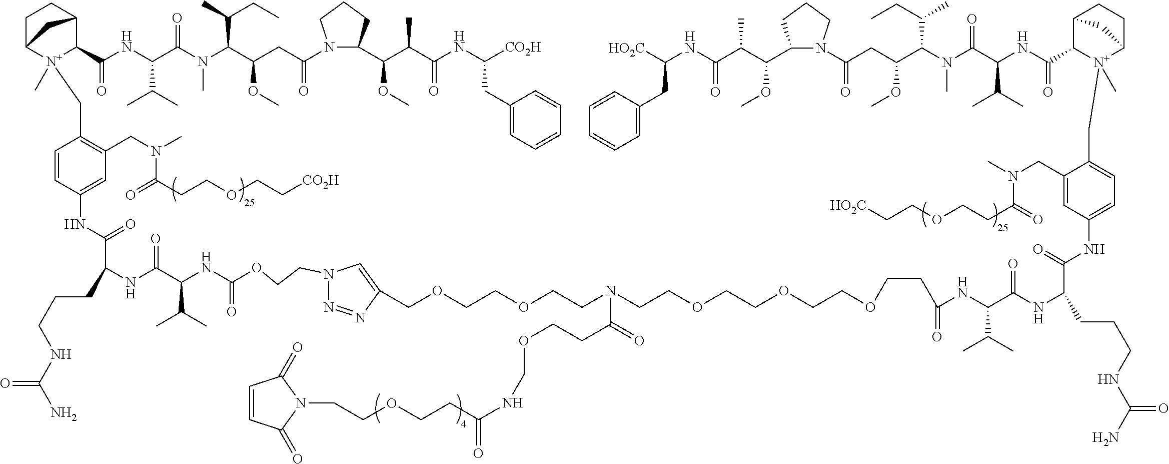 |
| P3- L34-P4 | 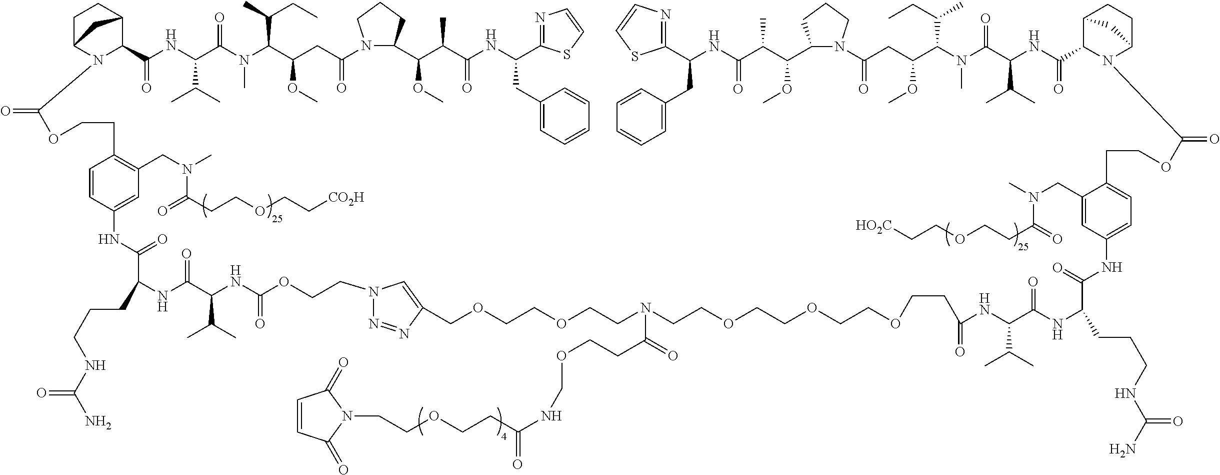 |
| P4- L34-P3 | 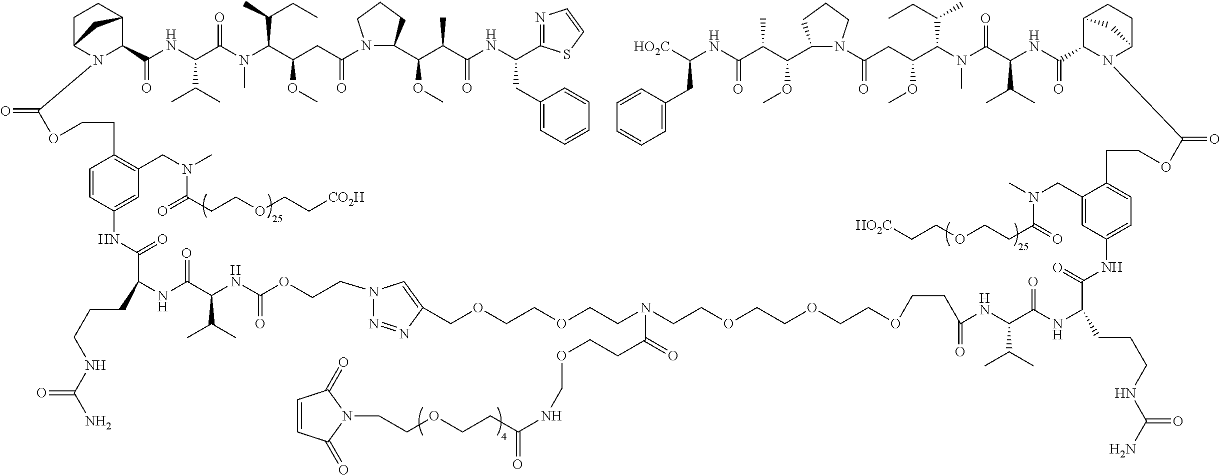 |
| P4 L34-P4 | 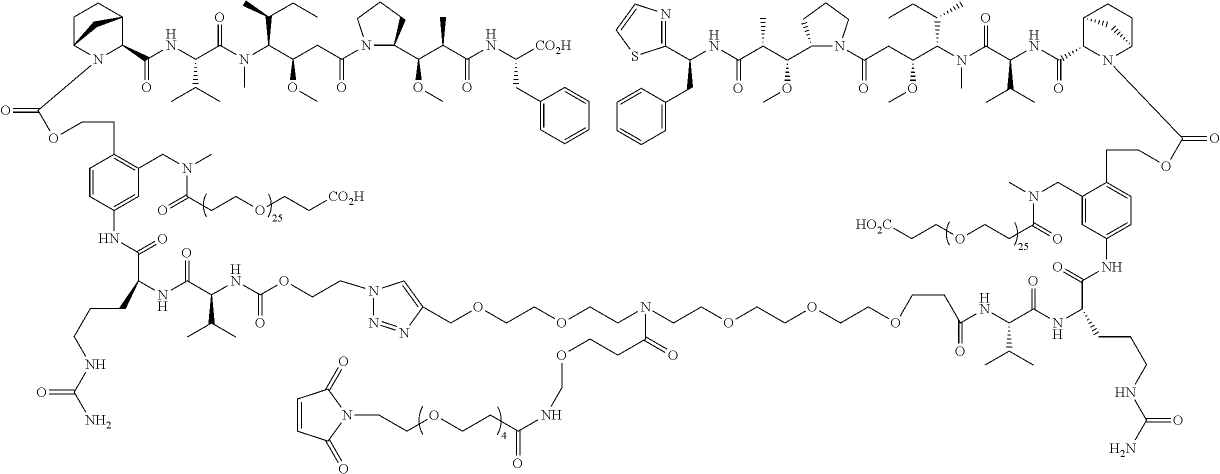 |
| P1- L35-P1 | 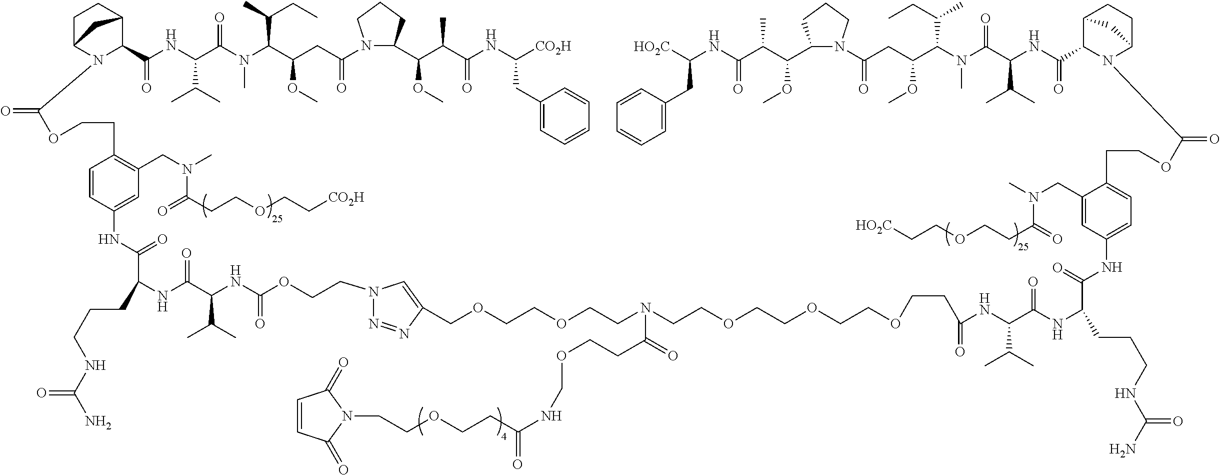 |
| P1- L35-P2 | 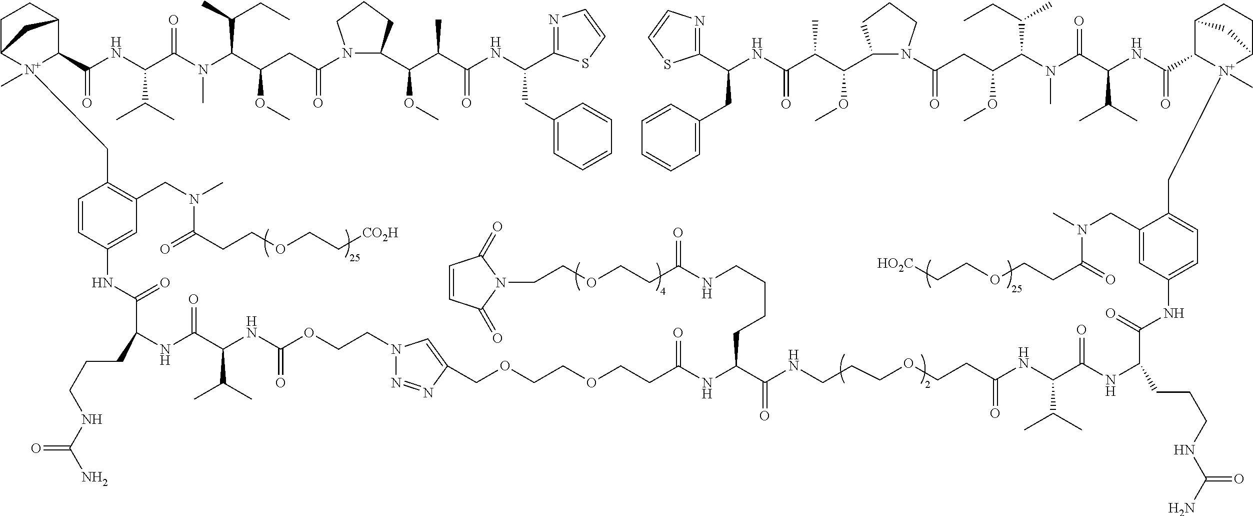 |
| P2- L35-P1 | 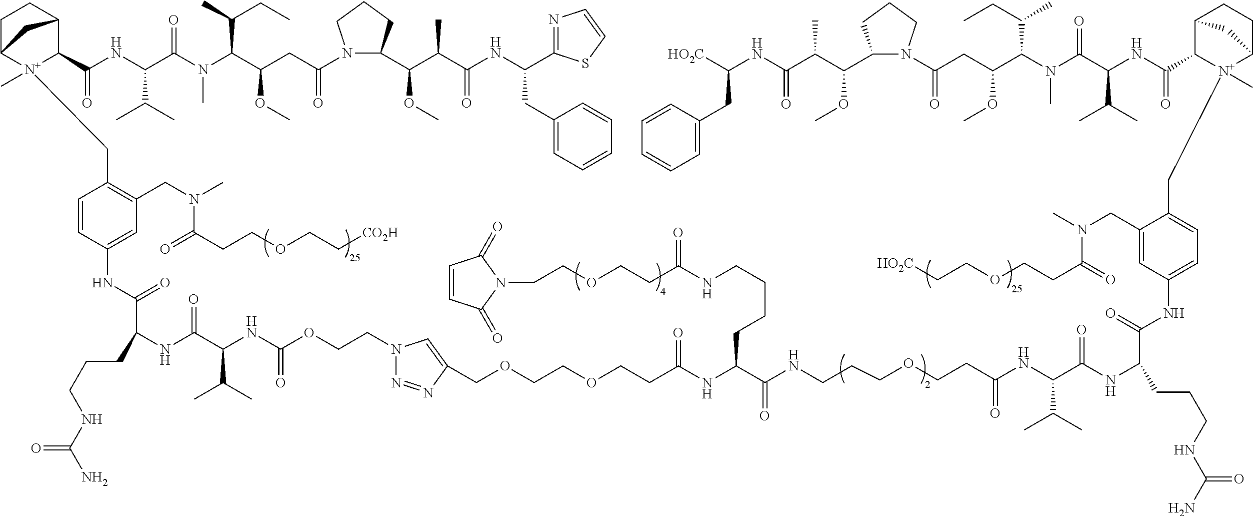 |
| P2- L35-P2 | 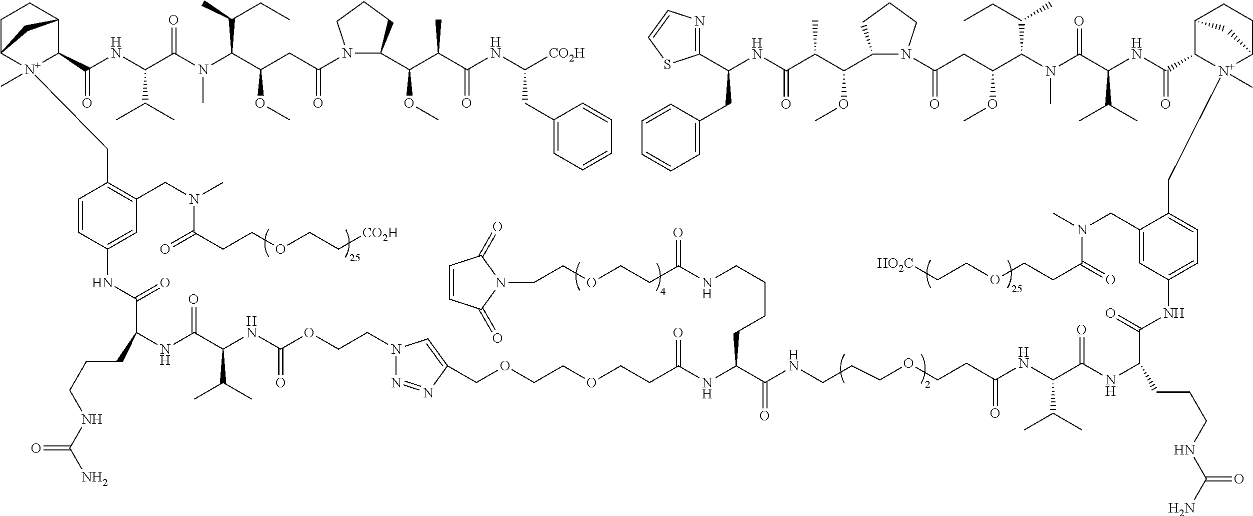 |
| P3- L35-P3 | 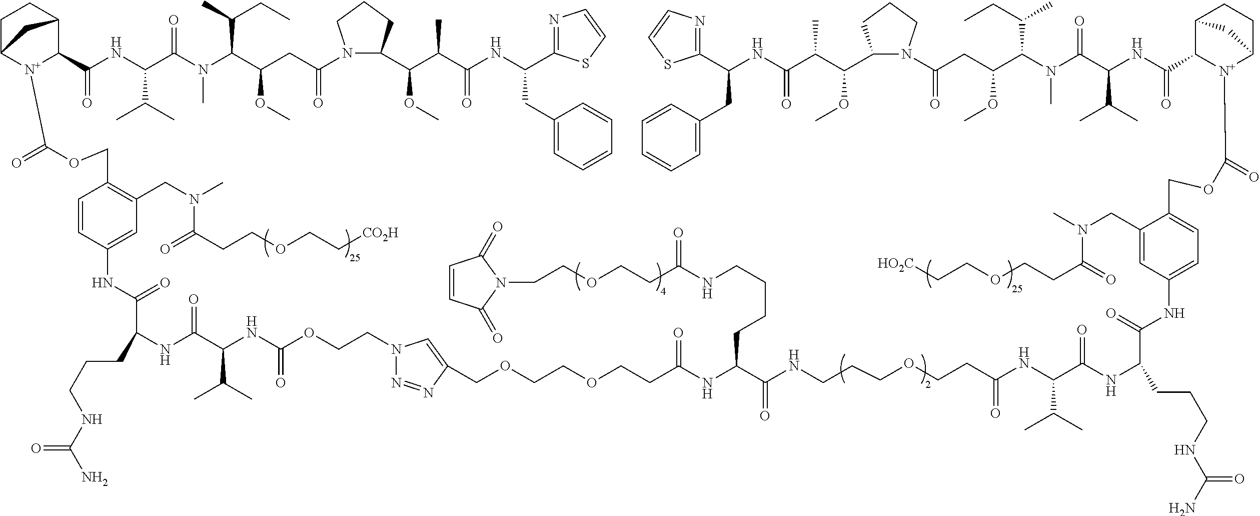 |
| P3- L35-P4 | 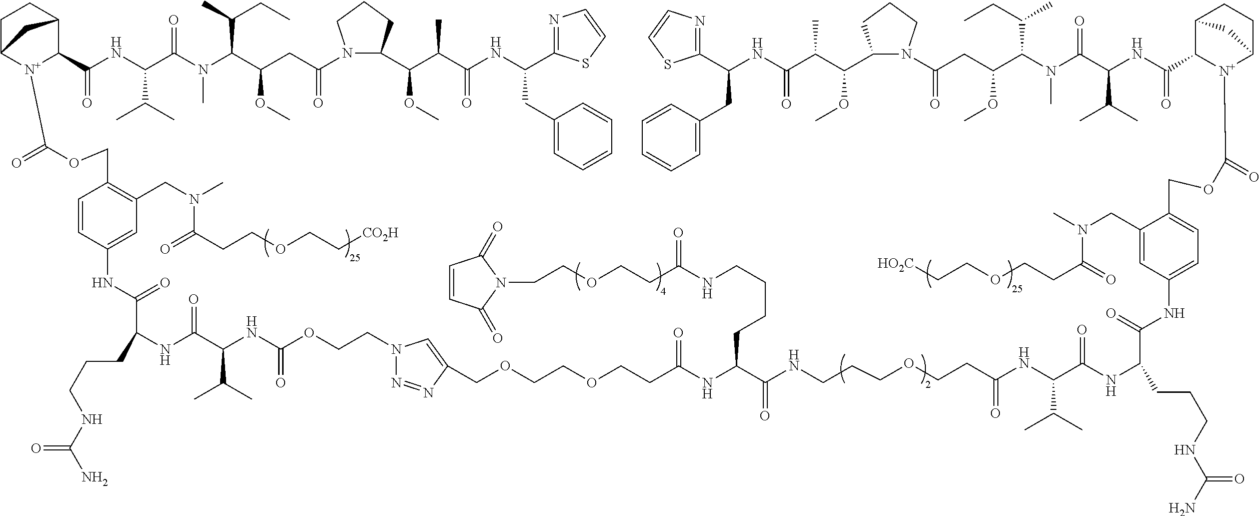 |
| P4- L35-P3 | 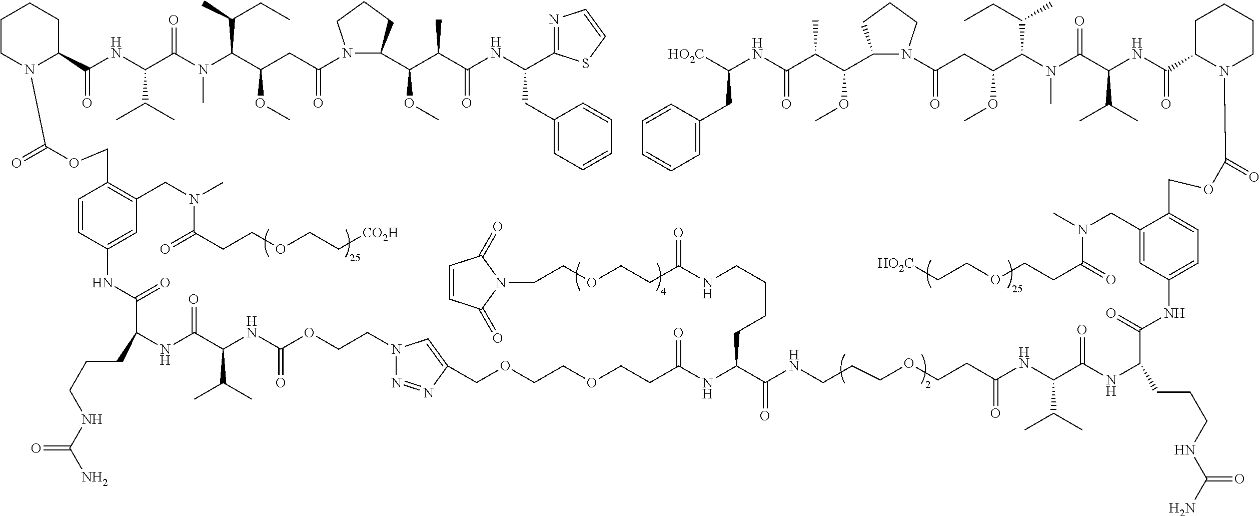 |
| P4- L35-P4 | 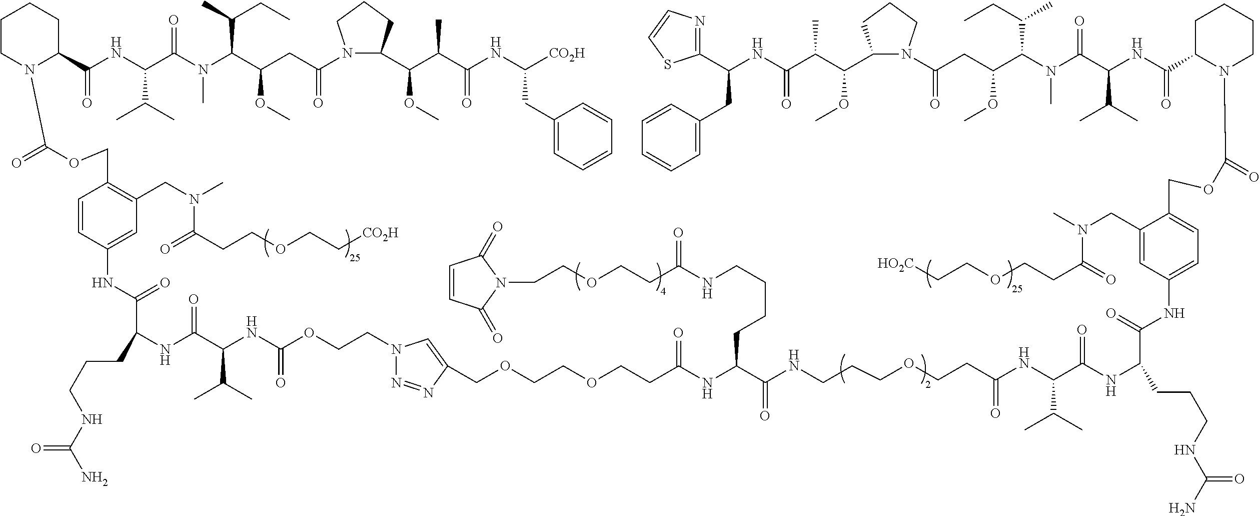 |
| P1- L36-P1 | 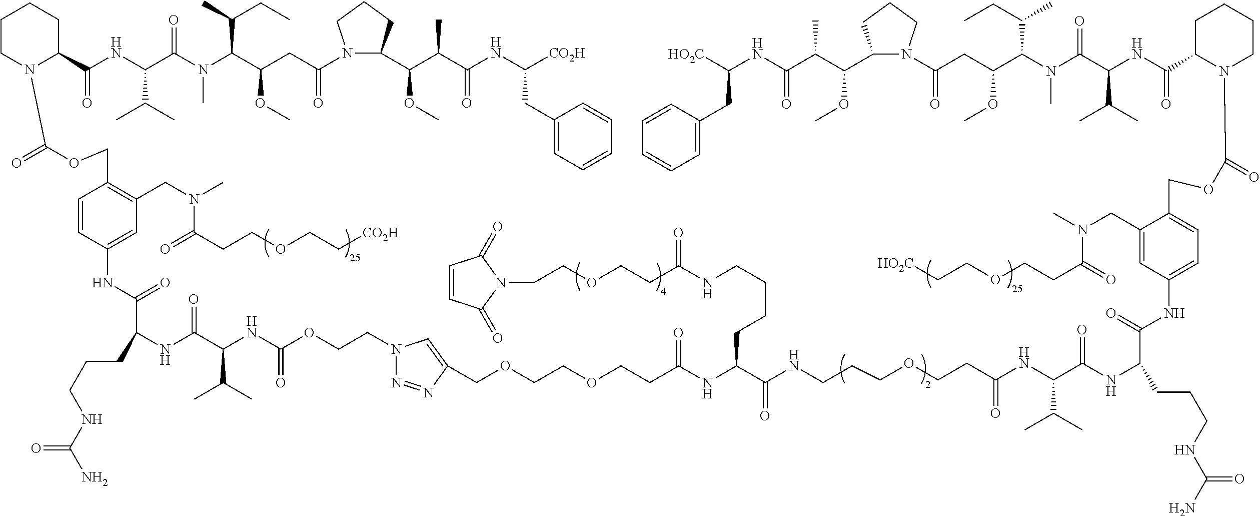 |
| P1- L36-P2 | 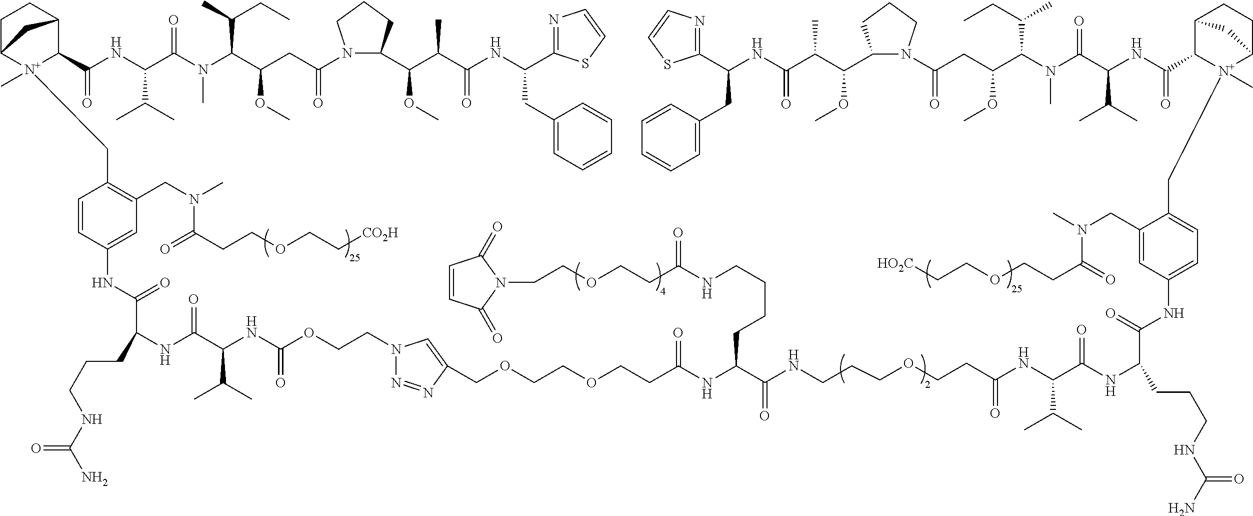 |
| P2- L36-P1 | 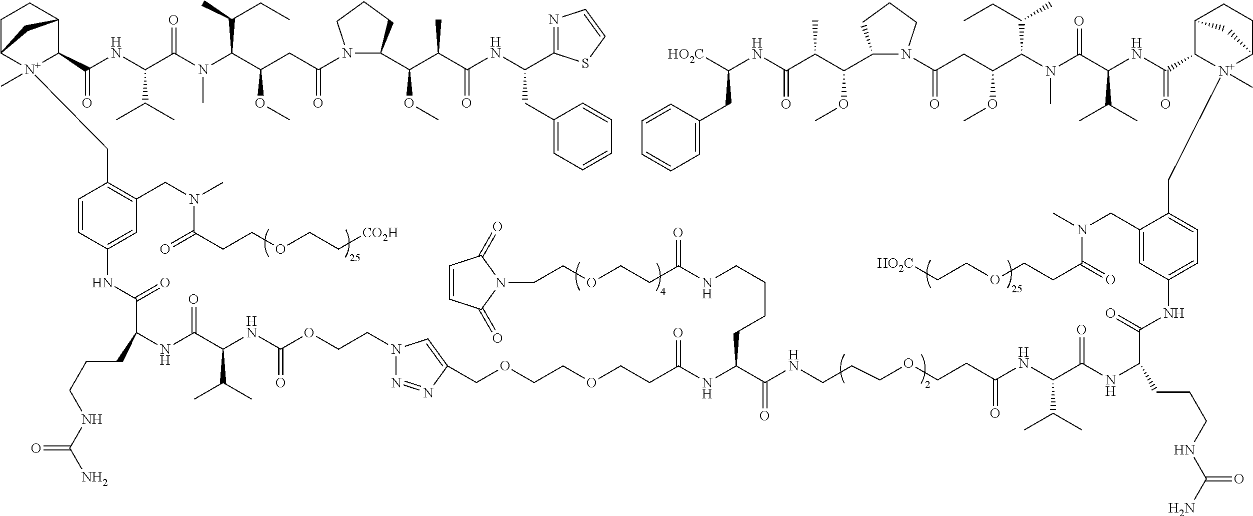 |
| P2- L36-P2 | 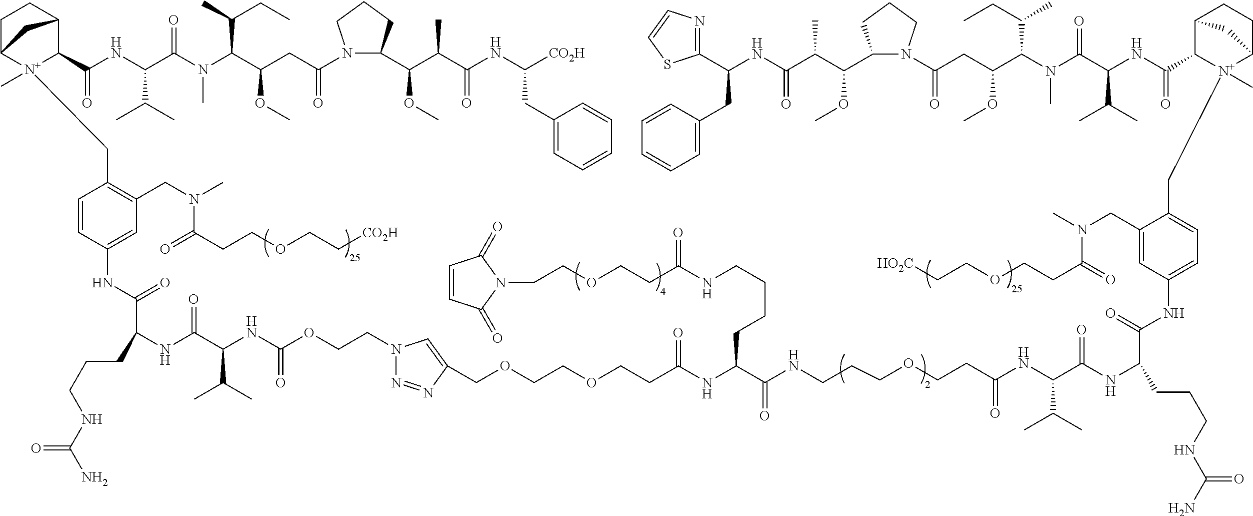 |
| P3- L36-P3 | 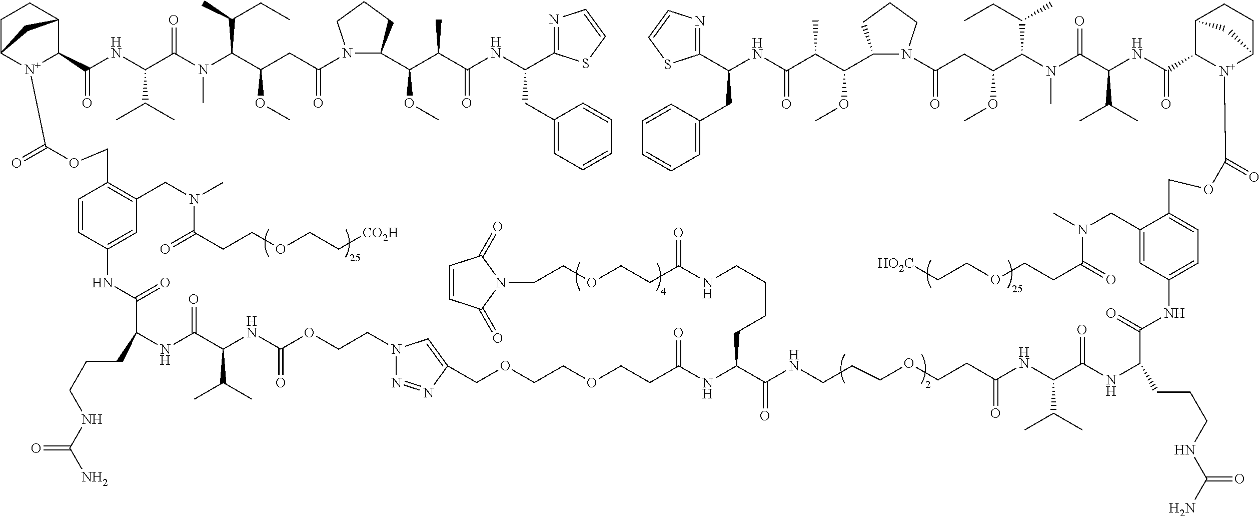 |
| P3- L36-P4 | 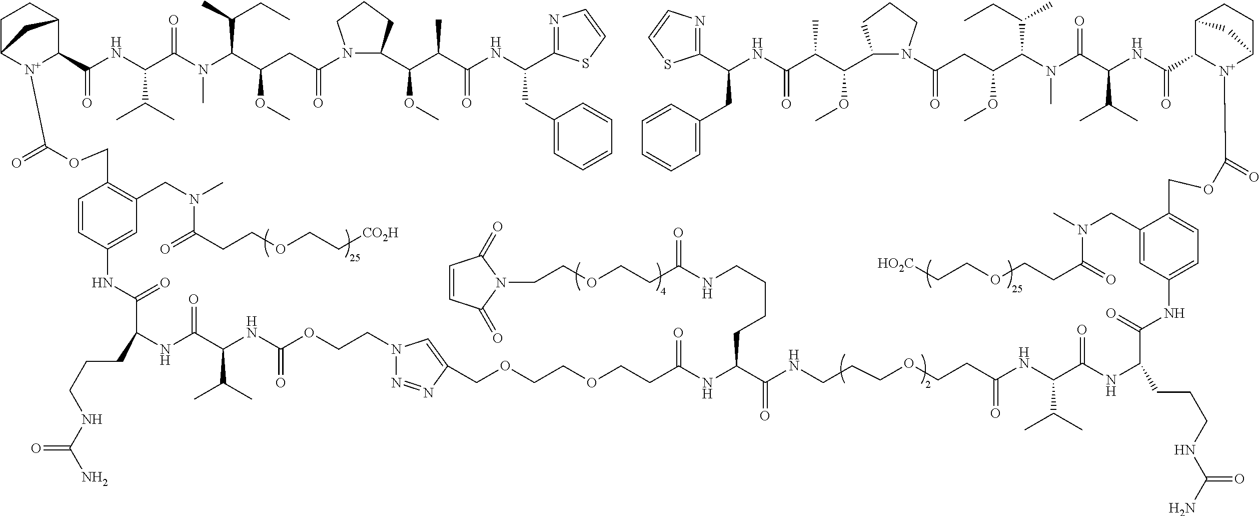 |
| P4- L36-P3 | 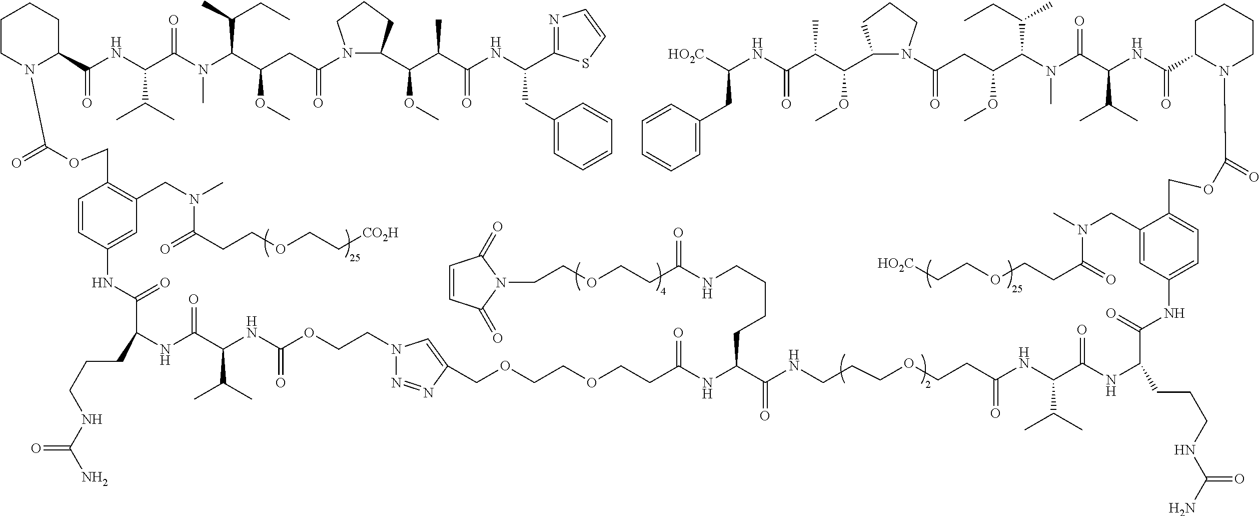 |
| P4- L36-P4 | 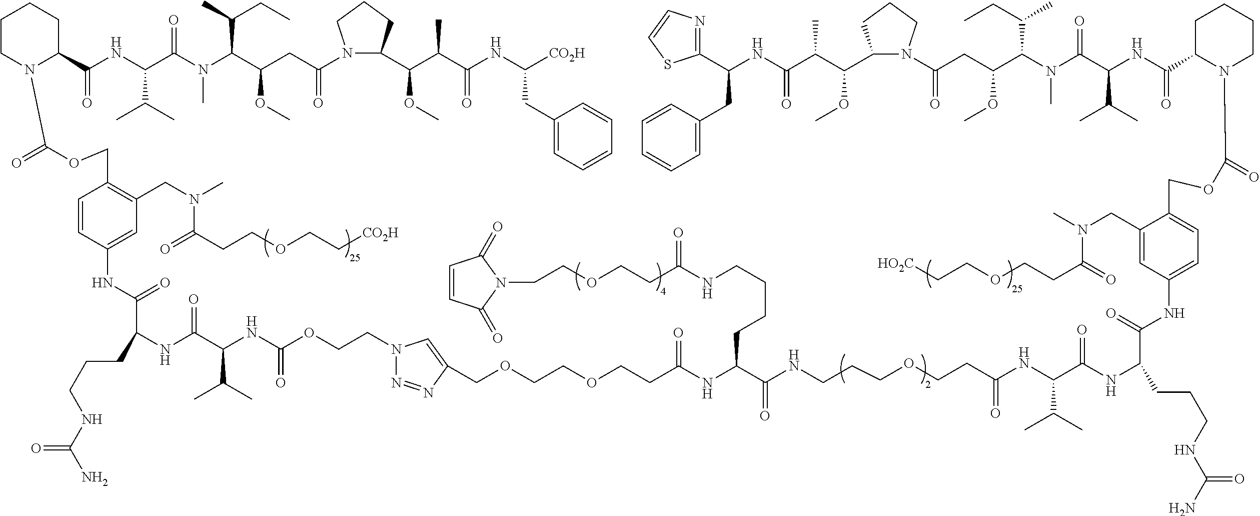 |
| P5- L36-P5 | 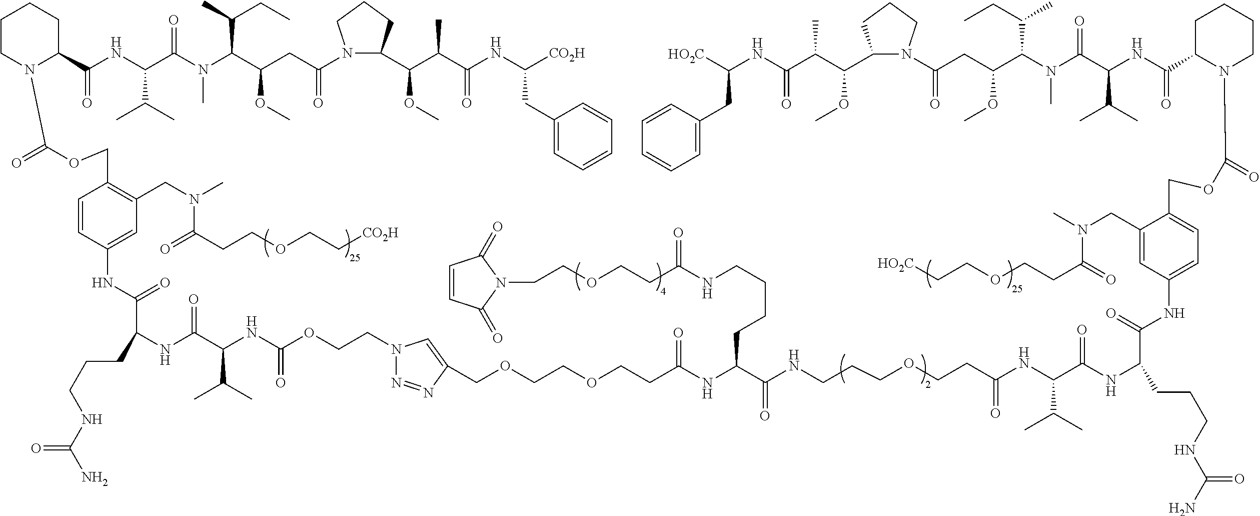 |
| P6- L36-P6 | 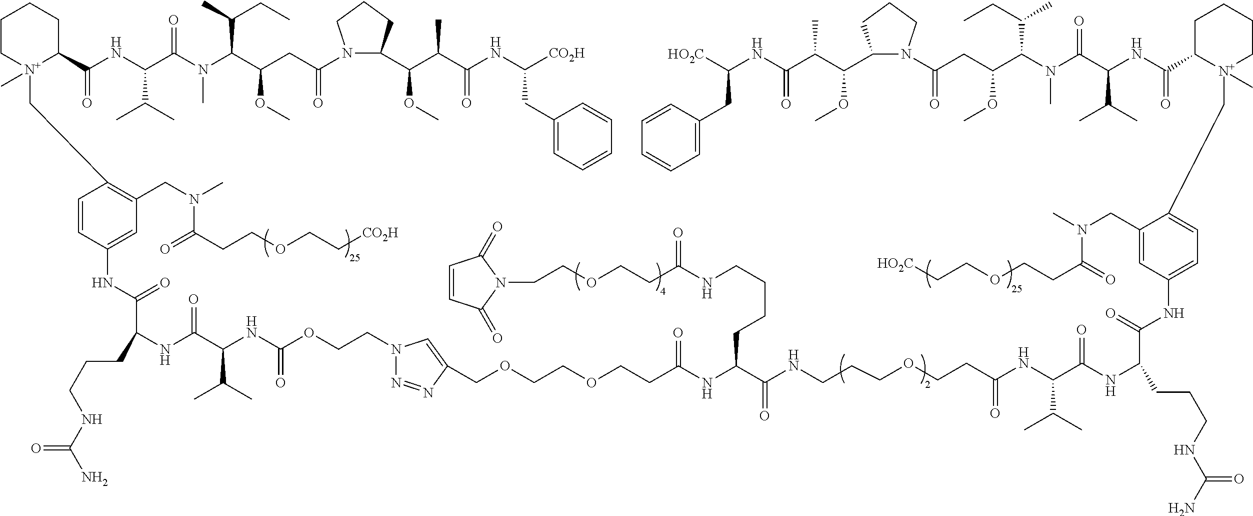 |
| Ex- ample #1 | 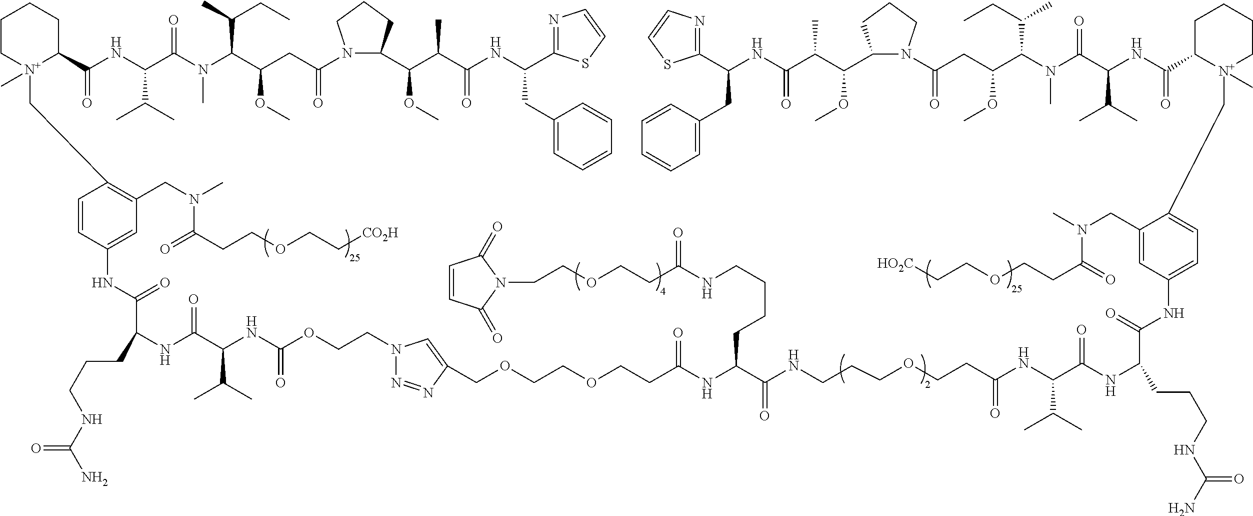 |

Using the procedures described in preparation of Examples #1-8, the following compounds could be prepared: P1-L1-P1, P1-L1-P2, P1-L1-P3, P1-L1-P4, P1-L1-P5, P1-L1-P6, P1-L1-P7, P1-L1-P8, P1-L2-P1, P1-L2-P2, P1-L2-P3, P1-L2-P4, P1-L2-P5, P1-L2-P6, P1-L2-P7, P1-L2-P8, P1-L3-P1, P1-L3-P2, P1-L3-P3, P1-L3-P4, P1-L3-P5, P1-L3-P6, P1-L3-P7, P1-L3-P8, P1-L4-P1, P1-L4-P2, P1-L4-P3, P1-L4-P4, P1-L4-P5, P1-L4-P6, P1-L4-P7, P1-L4-P8, P1-L5-P1, P1-L5-P2, P1-L5-P3, P1-L5-P4, P1-L5-P5, P1-L5-P6, P1-L5-P7, P1-L5-P8, P1-L6-P1, P1-L6-P2, P1-L6-P3, P1-L6-P4, P1-L6-P5, P1-L6-P6, P1-L6-P7, P1-L6-P8, P1-L7-P1, P1-L7-P2, P1-L7-P3, P1-L7-P4, P1-L7-P5, P1-L7-P6, P1-L7-P7, P1-L7-P8, P1-L8-P1, P1-L8-P2, P1-L8-P3, P1-L8-P4, P1-L8-P5, P1-L8-P6, P1-L8-P7, P1-L8-P8, P1-L9-P1, P1-L9-P2, P1-L9-P3, P1-L9-P4, P1-L9-P5, P1-L9-P6, P1-L9-P7, P1-L9-P8, P1-L10-P1, P1-10-P2, P1-L10-P3, P1-L10- P4, P1-L10-P5, P1-10-P6, P1-L10-P7, P1-10-P8, P1-L11-P1, P1-L11-P2, P1-L11-P3, P1-L11-P4, P1-L11-P5, P1-L11-P6, P1-L11-P7, P1-L11-P8, P1-L12-P1, P1-L12-P2, P1-L12-P3, P1-L12-P4, P1-L12-P5, P1-L12-P6, P1-L12-P7, P1-L12-P8, P1-L13-P1, P1-L13-P2, P1-L13-P3, P1-L13-P4, P1-L13-P5, P1-L13-P6, P1-L13-P7, P1-L13-P8, P1-L14-P1, P1-L14-P2, P1-L14-P3, P1-L14-P4, P1-L14-P5, P1-L14-P6, P1-L14-P7, P1-L14-P8, P1-L15-P1, P1-L15-P2, P1-L15-P3, P1-L15-P4, P1-L15-P5, P1-L15-P6, P1-L15-P7, P1-L15-P8, P1-L16-P1, P1-L16-P2, P1-L16-P3, P1-L16-P4, P1-L16-P5, P1-L16-P6, P1-L16-P7, P1-L16-P8, P1-L17-P1, P1-L17-P2, P1-L17-P3, P1-L17-P4, P1-L17-P5, P1-L17-P6, P1-L17-P7, P1-L17-P8, P1-L18-P1, P1-L18-P2, P1-L18-P3, P1-L18-P4, P1-L18-P5, P1-L18-P6, P1-L18-P7, P1-L18-P8, P1-L19-P1, P1-L19-P2, P1-L19-P3, P1-L19-P4, P1-L19-P5, P1-L19-P6, P1-L19-P7, P1-L19-P8, P1-L20-P1, P1-L20-P2, P1-L20-P3, P1-L20-P4, P1-L20-P5, P1-L20-P6, P1-L20-P7, P1-L20-P8, P1-L21-P1, P1-L21-P2, P1-L21-P3, P1-L21-P4, P1-L21-P5, P1-L21-P6, P1-L21-P7, P1-L21-P8, P1-L22-P1, P1-L22-P2, P1-L22-P3, P1-L22-P4, P1-L22-P5, P1-L22-P6, P1-L22-P7, P1-L22-P8, P1-L23-P1, P1-L23-P2, P1-L23-P3, P1-L23-P4, P1-L23-P5, P1-L23-P6, P1-L23-P7, P1-L23-P8, P1-L24-P1, P1-L24-P2, P1-L24-P3, P1-L24-P4, P1-L24-P5, P1-L24-P6, P1-L24-P7, P1-L24-P8, P1-L25-P1, P1-L25-P2, P1-L25-P3, P1-L25-P4, P1-L25-P5, P1-L25-P6, P1-L25-P7, P1-L25-P8, P1-L26-P1, P1-L26-P2, P1-L26-P3, P1-L26-P4, P1-L26-P5, P1-L26-P6, P1-L26-P7, P1-L26-P8, P1-L27-P1, P1-L27-P2, P1-L27-P3, P1-L27-P4, P1-L27-P5, P1-L27-P6, P1-L27-P7, P1-L27-P8, P1-L28-P1, P1-L28-P2, P1-L28-P3, P1-L28-P4, P1-L28-P5, P1-L28-P6, P1-L28-P7, P1-L28-P8, P1-L29-P1, P1-L29-P2, P1-L29-P3, P1-L29-P4, P1-L29-P5, P1-L29-P6, P1-L29-P7, P1-L29-P8, P1-L30-P1, P1-L30-P2, P1-L30-P3, P1-L30-P4, P1-L30-P5, P1-L30-P6, P1-L30-P7, P1-L30-P8, P1-L31-P1, P1-L31-P2, P1-L31-P3, P1-L31-P4, P1-L31-P5, P1-L31-P6, P1-L31-P7, P1-L31-P8, P1-L32-P1, P1-L32-P2, P1-L32-P3, P1-L32-P4, P1-L32-P5, P1-L32-P6, P1-L32-P7, P1-L32-P8, P1-L33-P1, P1-L33-P2, P1-L33-P3, P1-L33-P4, P1-L33-P5, P1-L33-P6, P1-L33-P7, P1-L33-P8, P1-L34-P1, P1-L34-P2, P1-L34-P3, P1-L34-P4, P1-L34-P5, P1-L34-P6, P1-L34-P7, P1-L34-P8, P1-L35-P1, P1-L35-P2, P1-L35-P3, P1-L35-P4, P1-L35-P5, P1-L35-P6, P1-L35-P7, P1-L35-P8, P1-L36-P1, P1-L36-P2, P1-L36-P3, P1-L36-P4, P1-L36-P5, P1-L36-P6, P1-L36-P7, P1-L36-P8, P2-L1-P1, P2-L1-P2, P2-L1-P3, P2-L1-P4, P2-L1-P5, P2-L1-P6, P2-L1-P7, P2-L1-P8, P2-L2-P1, P2-L2-P2, P2-L2-P3, P2-L2-P4, P2-L2-P5, P2-L2-P6, P2-L2-P7, P2-L2-P8, P2-L3-P1, P2-L3-P2, P2-L3-P3, P2-L3-P4, P2-L3-P5, P2-L3-P6, P2-L3-P7, P2-L3-P8, P2-L4-P1, P2-L4-P2, P2-L4-P3, P2-L4-P4, P2-L4-P5, P2-L4-P6, P2-L4-P7, P2-L4-P8, P2-L5-P1, P2-L5-P2, P2-L5-P3, P2-L5-P4, P2-L5-P5, P2-L5-P6, P2-L5-P7, P2-L5-P8, P2-L6-P1, P2-L6-P2, P2-L6-P3, P2-L6-P4, P2-L6-P5, P2-L6-P6, P2-L6-P7, P2-L6-P8, P2-L7-P1, P2-L7-P2, P2-L7-P3, P2-L7-P4, P2-L7-P5, P2-L7-P6, P2-L7-P7, P2-L7-P8, P2-L8-P1, P2-L8-P2, P2-L8-P3, P2-L8-P4, P2-L8-P5, P2-L8-P6, P2-L8-P7, P2-L8-P8, P2-L9-P1, P2-L9-P2, P2-L9-P3, P2-L9-P4, P2-L9-P5, P2-L9-P6, P2-L9-P7, P2-L9-P8, P2-10-P1, P2-110-P2, P2-10-P3, P2-L10-P4, P2-10-P5, P2-10-P6, P2-10-P7, P2-10-P8, P2-L11-P1, P2-L11-P2, P2-L11-P3, P2-L11-P4, P2-L11-P5, P2-L11-P6, P2-L11-P7, P2-L11-P8, P2-L12-P1, P2-L12-P2, P2-L12-P3, P2-L12-P4, P2-L12-P5, P2-L12-P6, P2-L12-P7, P2-L12-P8, P2-L13-P1, P2-L13-P2, P2-L13-P3, P2-L13-P4, P2-L13-P5, P2-L13-P6, P2-L13-P7, P2-L13-P8, P2-L14-P1, P2-L14-P2, P2-L14-P3, P2-L14-P4, P2-L14-P5, P2-L14-P6, P2-L14-P7, P2-L14-P8, P2-L15-P1, P2-L15-P2, P2-L15-P3, P2-L15-P4, P2-L15-P5, P2-L15-P6, P2-L15-P7, P2-L15-P8, P2-L16-P1, P2-L16-P2, P2-L16-P3, P2-L16-P4, P2-L16-P5, P2-L16-P6, P2-L16-P7, P2-L16-P8, P2-L17-P1, P2-L17-P2, P2-L17-P3, P2-L17-P4, P2-L17-P5, P2-L17-P6, P2-L17-P7, P2-L17-P8, P2-L18-P1, P2-L18-P2, P2-L18-P3, P2-L18-P4, P2-L18-P5, P2-L18-P6, P2-L18-P7, P2-L18-P8, P2-L19-P1, P2-L19-P2, P2-L19-P3, P2-L19-P4, P2-L19-P5, P2-L19-P6, P2-L19-P7, P2-L19-P8, P2-L20-P1, P2-L20-P2, P2-L20-P3, P2-L20-P4, P2-L20-P5, P2-L20-P6, P2-L20-P7, P2-L20-P8, P2-L21-P1, P2-L21-P2, P2-L21-P3, P2-L21-P4, P2-L21-P5, P2-L21-P6, P2-L21-P7, P2-L21-P8, P2-L22-P1, P2-L22-P2, P2-L22-P3, P2-L22-P4, P2-L22-P5, P2-L22-P6, P2-L22-P7, P2-L22-P8, P2-L23-P1, P2-L23-P2, P2-L23-P3, P2-L23-P4, P2-L23-P5, P2-L23-P6, P2-L23-P7, P2-L23-P8, P2-L24-P1, P2-L24-P2, P2-L24-P3, P2-L24-P4, P2-L24-P5, P2-L24-P6, P2-L24-P7, P2-L24-P8, P2-L25-P1, P2-L25-P2, P2-L25-P3, P2-L25-P4, P2-L25-P5, P2-L25-P6, P2-L25-P7, P2-L25-P8, P2-L26-P1, P2-L26-P2, P2-L26-P3, P2-L26-P4, P2-L26-P5, P2-L26-P6, P2-L26-P7, P2-L26-P8, P2-L27-P1, P2-L27-P2, P2-L27-P3, P2-L27-P4, P2-L27-P5, P2-L27-P6, P2-L27-P7, P2-L27-P8, P2-L28-P1, P2-L28-P2, P2-L28-P3, P2-L28-P4, P2-L28-P5, P2-L28-P6, P2-L28-P7, P2-L28-P8, P2-L29-P1, P2-L29-P2, P2-L29-P3, P2-L29-P4, P2-L29-P5, P2-L29-P6, P2-L29-P7, P2-L29-P8, P2-L30-P1, P2-L30-P2, P2-L30-P3, P2-L30-P4, P2-L30-P5, P2-L30-P6, P2-L30-P7, P2-L30-P8, P2-L31-P1, P2-L31-P2, P2-L31-P3, P2-L31-P4, P2-L31-P5, P2-L31-P6, P2-L31-P7, P2-L31-P8, P2-L32-P1, P2-L32-P2, P2-L32-P3, P2-L32-P4, P2-L32-P5, P2-L32-P6, P2-L32-P7, P2-L32-P8, P2-L33-P1, P2-L33-P2, P2-L33-P3, P2-L33-P4, P2-L33-P5, P2-L33-P6, P2-L33-P7, P2-L33-P8, P2-L34-P1, P2-L34-P2, P2-L34-P3, P2-L34-P4, P2-L34-P5, P2-L34-P6, P2-L34-P7, P2-L34-P8, P2-L35-P1, P2-L35-P2, P2-L35-P3, P2-L35-P4, P2-L35-P5, P2-L35-P6, P2-L35-P7, P2-L35-P8, P2-L36-P1, P2-L36-P2, P2-L36-P3, P2-L36-P4, P2-L36-P5, P2-L36-P6, P2-L36-P7, P2-L36-P8, P3-L1-P1, P3-L1-P2, P3-L1-P3, P3-L1-P4, P3-L1-P5, P3-L1-P6, P3-L1-P7, P3-L1-P8, P3-L2-P1, P3-L2-P2, P3-L2-P3, P3-L2-P4, P3-L2-P5, P3-L2-P6, P3-L2-P7, P3-L2-P8, P3-L3-P1, P3-L3-P2, P3-L3-P3, P3-L3-P4, P3-L3-P5, P3-L3-P6, P3-L3-P7, P3-L3-P8, P3-L4-P1, P3-L4-P2, P3-L4-P3, P3-L4-P4, P3-L4-P5, P3-L4-P6, P3-L4-P7, P3-L4-P8, P3-L5-P1, P3-L5-P2, P3-L5-P3, P3-L5-P4, P3-L5-P5, P3-L5-P6, P3-L5-P7, P3-L5-P8, P3-L6-P1, P3-L6-P2, P3-L6-P3, P3-L6-P4, P3-L6-P5, P3-L6-P6, P3-L6-P7, P3-L6-P8, P3-L7-P1, P3-L7-P2, P3-L7-P3, P3-L7-P4, P3-L7-P5, P3-L7-P6, P3-L7-P7, P3-L7-P8, P3-L8-P1, P3-L8-P2, P3-L8-P3, P3-L8-P4, P3-L8-P5, P3-L8-P6, P3-L8-P7, P3-L8-P8, P3-L9-P1, P3-L9-P2, P3-L9-P3, P3-L9-P4, P3-L9-P5, P3-L9-P6, P3-L9-P7, P3-L9-P8, P3-L10-P1, P3-L10-P2, P3-L10-P3, P3-L10-P4, P3-L10-P5, P3-L10-P6, P3-L10-P7, P3-L10-P8, P3-11-P1, P3-L11-P2, P3-L11-P3, P3-L11-P4, P3-L11-P5, P3-L11-P6, P3-L11-P7, P3-L11-P8, P3-L12-P1, P3-L12-P2, P3-L12-P3, P3-L12-P4, P3-L12-P5, P3-L12-P6, P3-L12-P7, P3-L12-P8, P3-L13-P1, P3-L13-P2, P3-L13-P3, P3-L13-P4, P3-L13-P5, P3-L13-P6, P3-L13-P7, P3-L13-P8, P3-L14-P1, P3-L14-P2, P3-L14-P3, P3-L14-P4, P3-L14-P5, P3-L14-P6, P3-L14-P7, P3-L14-P8, P3-L15-P1, P3-L15-P2, P3-L15-P3, P3-L15-P4, P3-L15-P5, P3-L15-P6, P3-L15-P7, P3-L15-P8, P3-L16-P1, P3-L16-P2, P3-L16-P3, P3-L16-P4, P3-L16-P5, P3-L16-P6, P3-L16-P7, P3-L16-P8, P3-L17-P1, P3-L17-P2, P3-L17-P3, P3-L17-P4, P3-L17-P5, P3-L17-P6, P3-L17-P7, P3-L17-P8, P3-L18-P1, P3-L18-P2, P3-L18-P3, P3-L18-P4, P3-L18-P5, P3-L18-P6, P3-L18-P7, P3-L18-P8, P3-L19-P1, P3-L19-P2, P3-L19-P3, P3-L19-P4, P3-L19-P5, P3-L19-P6, P3-L19-P7, P3-L19-P8, P3-L20-P1, P3-L20-P2, P3-L20-P3, P3-L20-P4, P3-L20-P5, P3-L20-P6, P3-L20-P7, P3-L20-P8, P3-L21-P1, P3-L21-P2, P3-L21-P3, P3-L21-P4, P3-L21-P5, P3-L21-P6, P3-L21-P7, P3-L21-P8, P3-L22-P1, P3-L22-P2, P3-L22-P3, P3-L22-P4, P3-L22-P5, P3-L22-P6, P3-L22-P7, P3-L22-P8, P3-L23-P1, P3-L23-P2, P3-L23-P3, P3-L23-P4, P3-L23-P5, P3-L23-P6, P3-L23-P7, P3-L23-P8, P3-L24-P1, P3-L24-P2, P3-L24-P3, P3-L24-P4, P3-L24-P5, P3-L24-P6, P3-L24-P7, P3-L24-P8, P3-L25-P1, P3-L25-P2, P3-L25-P3, P3-L25-P4, P3-L25-P5, P3-L25-P6, P3-L25-P7, P3-L25-P8, P3-L26-P1, P3-L26-P2, P3-L26-P3, P3-L26-P4, P3-L26-P5, P3-L26-P6, P3-L26-P7, P3-L26-P8, P3-L27-P1, P3-L27-P2, P3-L27-P3, P3-L27-P4, P3-L27-P5, P3-L27-P6, P3-L27-P7, P3-L27-P8, P3-L28-P1, P3-L28-P2, P3-L28-P3, P3-L28-P4, P3-L28-P5, P3-L28-P6, P3-L28-P7, P3-L28-P8, P3-L29-P1, P3-L29-P2, P3-L29-P3, P3-L29-P4, P3-L29-P5, P3-L29-P6, P3-L29-P7, P3-L29-P8, P3-L30-P1, P3-L30-P2, P3-L30-P3, P3-L30-P4, P3-L30-P5, P3-L30-P6, P3-L30-P7, P3-L30-P8, P3-L31-P1, P3-L31-P2, P3-L31-P3, P3-L31-P4, P3-L31-P5, P3-L31-P6, P3-L31-P7, P3-L31-P8, P3-L32-P1, P3-L32-P2, P3-L32-P3, P3-L32-P4, P3-L32-P5, P3-L32-P6, P3-L32-P7, P3-L32-P8, P3-L33-P1, P3-L33-P2, P3-L33-P3, P3-L33-P4, P3-L33-P5, P3-L33-P6, P3-L33-P7, P3-L33-P8, P3-L34-P1, P3-L34-P2, P3-L34-P3, P3-L34-P4, P3-L34-P5, P3-L34-P6, P3-L34-P7, P3-L34-P8, P3-L35-P1, P3-L35-P2, P3-L35-P3, P3-L35-P4, P3-L35-P5, P3-L35-P6, P3-L35-P7, P3-L35-P8, P3-L36-P1, P3-L36-P2, P3-L36-P3, P3-L36-P4, P3-L36-P5, P3-L36-P6, P3-L36-P7, P3-L36-P8, P4-L1-P1, P4-L1-P2, P4-L1-P3, P4-L1-P4, P4-L1-P5, P4-L1-P6, P4-L1-P7, P4-L1-P8, P4-L2-P1, P4-L2-P2, P4-L2-P3, P4-L2-P4, P4-L2-P5, P4-L2-P6, P4-L2-P7, P4-L2-P8, P4-L3-P1, P4-L3-P2, P4-L3-P3, P4-L3-P4, P4-L3-P5, P4-L3-P6, P4-L3-P7, P4-L3-P8, P4-L4-P1, P4-L4-P2, P4-L4-P3, P4-L4-P4, P4-L4-P5, P4-L4-P6, P4-L4-P7, P4-L4-P8, P4-L5-P1, P4-L5-P2, P4-L5-P3, P4-L5-P4, P4-L5-P5, P4-L5-P6, P4-L5-P7, P4-L5-P8, P4-L6-P1, P4-L6-P2, P4-L6-P3, P4-L6-P4, P4-L6-P5, P4-L6-P6, P4-L6-P7, P4-L6-P8, P4-L7-P1, P4-L7-P2, P4-L7-P3, P4-L7-P4, P4-L7-P5, P4-L7-P6, P4-L7-P7, P4-L7-P8, P4-L8-P1, P4-L8-P2, P4-L8-P3, P4-L8-P4, P4-L8-P5, P4-L8-P6, P4-L8-P7, P4-L8-P8, P4-L9-P1, P4-L9-P2, P4-L9-P3, P4-L9-P4, P4-L9-P5, P4-L9-P6, P4-L9-P7, P4-L9-P8, P4-L10-P1, P4-L10-P2, P4-L10-P3, P4- L10-P4, P4-10-P5, P4-10-P6, P4-10-P7, P4-10-P8, P4-L11-P1, P4-L11-P2, P4-L11-P3, P4-L11-P4, P4-L11-P5, P4-L11-P6, P4-L11-P7, P4-L11-P8, P4-L12-P1, P4-L12-P2, P4-L12-P3, P4-L12-P4, P4-L12-P5, P4-L12-P6, P4-L12-P7, P4-L12-P8, P4-L13-P1, P4-L13-P2, P4-L13-P3, P4-L13-P4, P4-L13-P5, P4-L13-P6, P4-L13-P7, P4-L13-P8, P4-L14-P1, P4-L14-P2, P4-L14-P3, P4-L14-P4, P4-L14-P5, P4-L14-P6, P4-L14-P7, P4-L14-P8, P4-L15-P1, P4-L15-P2, P4-L15-P3, P4-L15-P4, P4-L15-P5, P4-L15-P6, P4-L15-P7, P4-L15-P8, P4-L16-P1, P4-L16-P2, P4-L16-P3, P4-L16-P4, P4-L16-P5, P4-L16-P6, P4-L16-P7, P4-L16-P8, P4-L17-P1, P4-L17-P2, P4-L17-P3, P4-L17-P4, P4-L17-P5, P4-L17-P6, P4-L17-P7, P4-L17-P8, P4-L18-P1, P4-L18-P2, P4-L18-P3, P4-L18-P4, P4-L18-P5, P4-L18-P6, P4-L18-P7, P4-L18-P8, P4-L19-P1, P4-L19-P2, P4-L19-P3, P4-L19-P4, P4-L19-P5, P4-L19-P6, P4-L19-P7, P4-L19-P8, P4-L20-P1, P4-L20-P2, P4-L20-P3, P4-L20-P4, P4-L20-P5, P4-L20-P6, P4-L20-P7, P4-L20-P8, P4-L21-P1, P4-L21-P2, P4-L21-P3, P4-L21-P4, P4-L21-P5, P4-L21-P6, P4-L21-P7, P4-L21-P8, P4-L22-P1, P4-L22-P2, P4-L22-P3, P4-L22-P4, P4-L22-P5, P4-L22-P6, P4-L22-P7, P4-L22-P8, P4-L23-P1, P4-L23-P2, P4-L23-P3, P4-L23-P4, P4-L23-P5, P4-L23-P6, P4-L23-P7, P4-L23-P8, P4-L24-P1, P4-L24-P2, P4-L24-P3, P4-L24-P4, P4-L24-P5, P4-L24-P6, P4-L24-P7, P4-L24-P8, P4-L25-P1, P4-L25-P2, P4-L25-P3, P4-L25-P4, P4-L25-P5, P4-L25-P6, P4-L25-P7, P4-L25-P8, P4-L26-P1, P4-L26-P2, P4-L26-P3, P4-L26-P4, P4-L26-P5, P4-L26-P6, P4-L26-P7, P4-L26-P8, P4-L27-P1, P4-L27-P2, P4-L27-P3, P4-L27-P4, P4-L27-P5, P4-L27-P6, P4-L27-P7, P4-L27-P8, P4-L28-P1, P4-L28-P2, P4-L28-P3, P4-L28-P4, P4-L28-P5, P4-L28-P6, P4-L28-P7, P4-L28-P8, P4-L29-P1, P4-L29-P2, P4-L29-P3, P4-L29-P4, P4-L29-P5, P4-L29-P6, P4-L29-P7, P4-L29-P8, P4-L30-P1, P4-L30-P2, P4-L30-P3, P4-L30-P4, P4-L30-P5, P4-L30-P6, P4-L30-P7, P4-L30-P8, P4-L31-P1, P4-L31-P2, P4-L31-P3, P4-L31-P4, P4-L31-P5, P4-L31-P6, P4-L31-P7, P4-L31-P8, P4-L32-P1, P4-L32-P2, P4-L32-P3, P4-L32-P4, P4-L32-P5, P4-L32-P6, P4-L32-P7, P4-L32-P8, P4-L33-P1, P4-L33-P2, P4-L33-P3, P4-L33-P4, P4-L33-P5, P4-L33-P6, P4-L33-P7, P4-L33-P8, P4-L34-P1, P4-L34-P2, P4-L34-P3, P4-L34-P4, P4-L34-P5, P4-L34-P6, P4-L34-P7, P4-L34-P8, P4-L35-P1, P4-L35-P2, P4-L35-P3, P4-L35-P4, P4-L35-P5, P4-L35-P6, P4-L35-P7, P4-L35-P8, P4-L36-P1, P4-L36-P2, P4-L36-P3, P4-L36-P4, P4-L36-P5, P4-L36-P6, P4-L36-P7, P4-L36-P8, P5-L1-P1, P5-L1-P2, P5-L1-P3, P5-L1-P4, P5-L1-P5, P5-L1-P6, P5-L1-P7, P5-L1-P8, P5-L2-P1, P5-L2-P2, P5-L2-P3, P5-L2-P4, P5-L2-P5, P5-L2-P6, P5-L2-P7, P5-L2-P8, P5-L3-P1, P5-L3-P2, P5-L3-P3, P5-L3-P4, P5-L3-P5, P5-L3-P6, P5-L3-P7, P5-L3-P8, P5-L4-P1, P5-L4-P2, P5-L4-P3, P5-L4-P4, P5-L4-P5, P5-L4-P6, P5-L4-P7, P5-L4-P8, P5-L5-P1, P5-L5-P2, P5-L5-P3, P5-L5-P4, P5-L5-P5, P5-L5-P6, P5-L5-P7, P5-L5-P8, P5-L6-P1, P5-L6-P2, P5-L6-P3, P5-L6-P4, P5-L6-P5, P5-L6-P6, P5-L6-P7, P5-L6-P8, P5-L7-P1, P5-L7-P2, P5-L7-P3, P5-L7-P4, P5-L7-P5, P5-L7-P6, P5-L7-P7, P5-L7-P8, P5-L8-P1, P5-L8-P2, P5-L8-P3, P5-L8-P4, P5-L8-P5, P5-L8-P6, P5-L8-P7, P5-L8-P8, P5-L9-P1, P5-L9-P2, P5-L9-P3, P5-L9-P4, P5-L9-P5, P5-L9-P6, P5-L9-P7, P5-L9-P8, P5-L10-P1, P5-L10-P2, P5-L10-P3, P5-L10-P4, P5-L10-P5, P5-L10-P6, P5-L10-P7, P5-L10-P8, P5-L11-P1, P5-L11-P2, P5-L11-P3, P5-L11-P4, P5-L11-P5, P5-L11-P6, P5-L11-P7, P5-L11-P8, P5-L12-P1, P5-L12-P2, P5-L12-P3, P5-L12-P4, P5-L12-P5, P5-L12-P6, P5-L12-P7, P5-L12-P8, P5-L13-P1, P5-L13-P2, P5-L13-P3, P5-L13-P4, P5-L13-P5, P5-L13-P6, P5-L13-P7, P5-L13-P8, P5-L14-P1, P5-L14-P2, P5-L14-P3, P5-L14-P4, P5-L14-P5, P5-L14-P6, P5-L14-P7, P5-L14-P8, P5-L15-P1, P5-L15-P2, P5-L15-P3, P5-L15-P4, P5-L15-P5, P5-L15-P6, P5-L15-P7, P5-L15-P8, P5-L16-P1, P5-L16-P2, P5-L16-P3, P5-L16-P4, P5-L16-P5, P5-L16-P6, P5-L16-P7, P5-L16-P8, P5-L17-P1, P5-L17-P2, P5-L17-P3, P5-L17-P4, P5-L17-P5, P5-L17-P6, P5-L17-P7, P5-L17-P8, P5-L18-P1, P5-L18-P2, P5-L18-P3, P5-L18-P4, P5-L18-P5, P5-L18-P6, P5-L18-P7, P5-L18-P8, P5-L19-P1, P5-L19-P2, P5-L19-P3, P5-L19-P4, P5-L19-P5, P5-L19-P6, P5-L19-P7, P5-L19-P8, P5-L20-P1, P5-L20-P2, P5-L20-P3, P5-L20-P4, P5-L20-P5, P5-L20-P6, P5-L20-P7, P5-L20-P8, P5-L21-P1, P5-L21-P2, P5-L21-P3, P5-L21-P4, P5-L21-P5, P5-L21-P6, P5-L21-P7, P5-L21-P8, P5-L22-P1, P5-L22-P2, P5-L22-P3, P5-L22-P4, P5-L22-P5, P5-L22-P6, P5-L22-P7, P5-L22-P8, P5-L23-P1, P5-L23-P2, P5-L23-P3, P5-L23-P4, P5-L23-P5, P5-L23-P6, P5-L23-P7, P5-L23-P8, P5-L24-P1, P5-L24-P2, P5-L24-P3, P5-L24-P4, P5-L24-P5, P5-L24-P6, P5-L24-P7, P5-L24-P8, P5-L25-P1, P5-L25-P2, P5-L25-P3, P5-L25-P4, P5-L25-P5, P5-L25-P6, P5-L25-P7, P5-L25-P8, P5-L26-P1, P5-L26-P2, P5-L26-P3, P5-L26-P4, P5-L26-P5, P5-L26-P6, P5-L26-P7, P5-L26-P8, P5-L27-P1, P5-L27-P2, P5-L27-P3, P5-L27-P4, P5-L27-P5, P5-L27-P6, P5-L27-P7, P5-L27-P8, P5-L28-P1, P5-L28-P2, P5-L28-P3, P5-L28-P4, P5-L28-P5, P5-L28-P6, P5-L28-P7, P5-L28-P8, P5-L29-P1, P5-L29-P2, P5-L29-P3, P5-L29-P4, P5-L29-P5, P5-L29-P6, P5-L29-P7, P5-L29-P8, P5-L30-P1, P5-L30-P2, P5-L30-P3, P5-L30-P4, P5-L30-P5, P5-L30-P6, P5-L30-P7, P5-L30-P8, P5-L31-P1, P5-L31-P2, P5-L31-P3, P5-L31-P4, P5-L31-P5, P5-L31-P6, P5-L31-P7, P5-L31-P8, P5-L32-P1, P5-L32-P2, P5-L32-P3, P5-L32-P4, P5-L32-P5, P5-L32-P6, P5-L32-P7, P5-L32-P8, P5-L33-P1, P5-L33-P2, P5-L33-P3, P5-L33-P4, P5-L33-P5, P5-L33-P6, P5-L33-P7, P5-L33-P8, P5-L34-P1, P5-L34-P2, P5-L34-P3, P5-L34-P4, P5-L34-P5, P5-L34-P6, P5-L34-P7, P5-L34-P8, P5-L35-P1, P5-L35-P2, P5-L35-P3, P5-L35-P4, P5-L35-P5, P5-L35-P6, P5-L35-P7, P5-L35-P8, P5-L36-P1, P5-L36-P2, P5-L36-P3, P5-L36-P4, P5-L36-P5, P5-L36-P6, P5-L36-P7, P5-L36-P8, P6-L1-P1, P6-L1-P2, P6-L1-P3, P6-L1-P4, P6-L1-P5, P6-L1-P6, P6-L1-P7, P6-L1-P8, P6-L2-P1, P6-L2-P2, P6-L2-P3, P6-L2-P4, P6-L2-P5, P6-L2-P6, P6-L2-P7, P6-L2-P8, P6-L3-P1, P6-L3-P2, P6-L3-P3, P6-L3-P4, P6-L3-P5, P6-L3-P6, P6-L3-P7, P6-L3-P8, P6-L4-P1, P6-L4-P2, P6-L4-P3, P6-L4-P4, P6-L4-P5, P6-L4-P6, P6-L4-P7, P6-L4-P8, P6-L5-P1, P6-L5-P2, P6-15-P3, P6-L5-P4, P6-L5-P5, P6-L5-P6, P6-L5-P7, P6-L5-P8, P6-L6-P1, P6-L6-P2, P6-L6-P3, P6-L6-P4, P6-L6-P5, P6-L6-P6, P6-L6-P7, P6-L6-P8, P6-L7-P1, P6-L7-P2, P6-L7-P3, P6-L7-P4, P6-L7-P5, P6-L7-P6, P6-L7-P7, P6-L7-P8, P6-L8-P1, P6-L8-P2, P6-L8-P3, P6-L8-P4, P6-L8-P5, P6-L8-P6, P6-L8-P7, P6-L8-P8, P6-L9-P1, P6-L9-P2, P6-L9-P3, P6-L9-P4, P6-L9-P5, P6-L9-P6, P6-L9-P7, P6-L9-P8, P6-10-P1, P6-10-P2, P6-L10-P3, P6-L10-P4, P6-L10-P5, P6-L10-P6, P6-10-P7, P6-10-P8, P6-11-P1, P6-L11-P2, P6-L11-P3, P6-L11-P4, P6-L11-P5, P6-L11-P6, P6-L11-P7, P6-L11-P8, P6-L12-P1, P6-L12-P2, P6-L12-P3, P6-L12-P4, P6-L12-P5, P6-L12-P6, P6-L12-P7, P6-L12-P8, P6-L13-P1, P6-L13-P2, P6-L13-P3, P6-L13-P4, P6-L13-P5, P6-L13-P6, P6-L13-P7, P6-L13-P8, P6-L14-P1, P6-L14-P2, P6-L14-P3, P6-L14-P4, P6-L14-P5, P6-L14-P6, P6-L14-P7, P6-L14-P8, P6-15-P1, P6-15-P2, P6-15-P3, P6-L15-P4, P6-15-P5, P6-15-P6, P6-15-P7, P6-15-P8, P6-L16-P1, P6-L16-P2, P6-L16-P3, P6-L16-P4, P6-L16-P5, P6-L16-P6, P6-L16-P7, P6-L16-P8, P6-L17-P1, P6-L17-P2, P6-L17-P3, P6-L17-P4, P6-L17-P5, P6-L17-P6, P6-L17-P7, P6-L17-P8, P6-L18-P1, P6-L18-P2, P6-L18-P3, P6-L18-P4, P6-L18-P5, P6-L18-P6, P6-L18-P7, P6-L18-P8, P6-L19-P1, P6-L19-P2, P6-L19-P3, P6-L19-P4, P6-L19-P5, P6-L19-P6, P6-L19-P7, P6-L19-P8, P6-L20-P1, P6-L20-P2, P6-L20-P3, P6-L20-P4, P6-L20-P5, P6-L20-P6, P6-L20-P7, P6-L20-P8, P6-L21-P1, P6-L21-P2, P6-L21-P3, P6-L21-P4, P6-L21-P5, P6-L21-P6, P6-L21-P7, P6-L21-P8, P6-L22-P1, P6-L22-P2, P6-L22-P3, P6-L22-P4, P6-L22-P5, P6-L22-P6, P6-L22-P7, P6-L22-P8, P6-L23-P1, P6-L23-P2, P6-L23-P3, P6-L23-P4, P6-L23-P5, P6-L23-P6, P6-L23-P7, P6-L23-P8, P6-L24-P1, P6-L24-P2, P6-L24-P3, P6-L24-P4, P6-L24-P5, P6-L24-P6, P6-L24-P7, P6-L24-P8, P6-L25-P1, P6-L25-P2, P6-L25-P3, P6-L25-P4, P6-L25-P5, P6-L25-P6, P6-L25-P7, P6-L25-P8, P6-L26-P1, P6-L26-P2, P6-L26-P3, P6-L26-P4, P6-L26-P5, P6-L26-P6, P6-L26-P7, P6-L26-P8, P6-L27-P1, P6-L27-P2, P6-L27-P3, P6-L27-P4, P6-L27-P5, P6-L27-P6, P6-L27-P7, P6-L27-P8, P6-L28-P1, P6-L28-P2, P6-L28-P3, P6-L28-P4, P6-L28-P5, P6-L28-P6, P6-L28-P7, P6-L28-P8, P6-L29-P1, P6-L29-P2, P6-L29-P3, P6-L29-P4, P6-L29-P5, P6-L29-P6, P6-L29-P7, P6-L29-P8, P6-L30-P1, P6-L30-P2, P6-L30-P3, P6-L30-P4, P6-L30-P5, P6-L30-P6, P6-L30-P7, P6-L30-P8, P6-L31-P1, P6-L31-P2, P6-L31-P3, P6-L31-P4, P6-L31-P5, P6-L31-P6, P6-L31-P7, P6-L31-P8, P6-L32-P1, P6-L32-P2, P6-L32-P3, P6-L32-P4, P6-L32-P5, P6-L32-P6, P6-L32-P7, P6-L32-P8, P6-L33-P1, P6-L33-P2, P6-L33-P3, P6-L33-P4, P6-L33-P5, P6-L33-P6, P6-L33-P7, P6-L33-P8, P6-L34-P1, P6-L34-P2, P6-L34-P3, P6-L34-P4, P6-L34-P5, P6-L34-P6, P6-L34-P7, P6-L34-P8, P6-L35-P1, P6-L35-P2, P6-L35-P3, P6-L35-P4, P6-L35-P5, P6-L35-P6, P6-L35-P7, P6-L35-P8, P6-L36-P1, P6-L36-P2, P6-L36-P3, P6-L36-P4, P6-L36-P5, P6-L36-P6, P6-L36-P7, P6-L36-P8, P7-L1-P1, P7-L1-P2, P7-L1-P3, P7-L1-P4, P7-L1-P5, P7-L1-P6, P7-L1-P7, P7-L1-P8, P7-L2-P1, P7-L2-P2, P7-L2-P3, P7-L2-P4, P7-L2-P5, P7-L2-P6, P7-L2-P7, P7-L2-P8, P7-L3-P1, P7-L3-P2, P7-L3-P3, P7-L3-P4, P7-L3-P5, P7-L3-P6, P7-L3-P7, P7-L3-P8, P7-L4-P1, P7-L4-P2, P7-L4-P3, P7-L4-P4, P7-L4-P5, P7-L4-P6, P7-L4-P7, P7-L4-P8, P7-L5-P1, P7-L5-P2, P7-L5-P3, P7-L5-P4, P7-L5-P5, P7-L5-P6, P7-L5-P7, P7-L5-P8, P7-L6-P1, P7-L6-P2, P7-L6-P3, P7-L6-P4, P7-L6-P5, P7-L6-P6, P7-L6-P7, P7-L6-P8, P7-L7-P1, P7-L7-P2, P7-L7-P3, P7-L7-P4, P7-L7-P5, P7-L7-P6, P7-L7-P7, P7-L7-P8, P7-L8-P1, P7-L8-P2, P7-L8-P3, P7-L8-P4, P7-L8-P5, P7-L8-P6, P7-L8-P7, P7-L8-P8, P7-L9-P1, P7-L9-P2, P7-L9-P3, P7-L9-P4, P7-L9-P5, P7-L9-P6, P7-L9-P7, P7-L9-P8, P7-10-P1, P7-10-P2, P7-L10-P3, P7-L10-P4, P7-10-P5, P7-10-P6, P7-10-P7, P7-10-P8, P7-L11-P1, P7-L11-P2, P7-L11-P3, P7-L11-P4, P7-L11-P5, P7-L11-P6, P7-L11-P7, P7-L11-P8, P7-L12-P1, P7-L12-P2, P7-L12-P3, P7-L12-P4, P7-L12-P5, P7-L12-P6, P7-L12-P7, P7-L12-P8, P7-L13-P1, P7-L13-P2, P7-L13-P3, P7-L13-P4, P7-L13-P5, P7-L13-P6, P7-L13-P7, P7-L13-P8, P7-L14-P1, P7-L14-P2, P7-L14-P3, P7-L14-P4, P7-L14-P5, P7-L14-P6, P7-L14-P7, P7-L14-P8, P7-L15-P1, P7-L15-P2, P7-L15-P3, P7-L15-P4, P7-L15-P5, P7-L15-P6, P7-L15-P7, P7-L15-P8, P7-L16-P1, P7-L16-P2, P7-L16-P3, P7-L16-P4, P7-L16-P5, P7-L16-P6, P7-L16-P7, P7-L16-P8, P7-L17-P1, P7-L17-P2, P7-L17-P3, P7-L17-P4, P7-L17-P5, P7-L17-P6, P7-L17-P7, P7-L17-P8, P7-L18-P1, P7-L18-P2, P7-L18-P3, P7-L18-P4, P7-L18-P5, P7-L18-P6, P7-L18-P7, P7-L18-P8, P7-L19-P1, P7-L19-P2, P7-L19-P3, P7-L19-P4, P7-L19-P5, P7-L19-P6, P7-L19-P7, P7-L19-P8, P7-L20-P1, P7-L20-P2, P7-L20-P3, P7-L20-P4, P7-L20-P5, P7-L20-P6, P7-L20-P7, P7-L20-P8, P7-L21-P1, P7-L21-P2, P7-L21-P3, P7-L21-P4, P7-L21-P5, P7-L21-P6, P7-L21-P7, P7-L21-P8, P7-L22-P1, P7-L22-P2, P7-L22-P3, P7-L22-P4, P7-L22-P5, P7-L22-P6, P7-L22-P7, P7-L22-P8, P7-L23-P1, P7-L23-P2, P7-L23-P3, P7-L23-P4, P7-L23-P5, P7-L23-P6, P7-L23-P7, P7-L23-P8, P7-L24-P1, P7-L24-P2, P7-L24-P3, P7-L24-P4, P7-L24-P5, P7-L24-P6, P7-L24-P7, P7-L24-P8, P7-L25-P1, P7-L25-P2, P7-L25-P3, P7-L25-P4, P7-L25-P5, P7-L25-P6, P7-L25-P7, P7-L25-P8, P7-L26-P1, P7-L26-P2, P7-L26-P3, P7-L26-P4, P7-L26-P5, P7-L26-P6, P7-L26-P7, P7-L26-P8, P7-L27-P1, P7-L27-P2, P7-L27-P3, P7-L27-P4, P7-L27-P5, P7-L27-P6, P7-L27-P7, P7-L27-P8, P7-L28-P1, P7-L28-P2, P7-L28-P3, P7-L28-P4, P7-L28-P5, P7-L28-P6, P7-L28-P7, P7-L28-P8, P7-L29-P1, P7-L29-P2, P7-L29-P3, P7-L29-P4, P7-L29-P5, P7-L29-P6, P7-L29-P7, P7-L29-P8, P7-L30-P1, P7-L30-P2, P7-L30-P3, P7-L30-P4, P7-L30-P5, P7-L30-P6, P7-L30-P7, P7-L30-P8, P7-L31-P1, P7-L31-P2, P7-L31-P3, P7-L31-P4, P7-L31-P5, P7-L31-P6, P7-L31-P7, P7-L31-P8, P7-L32-P1, P7-L32-P2, P7-L32-P3, P7-L32-P4, P7-L32-P5, P7-L32-P6, P7-L32-P7, P7-L32-P8, P7-L33-P1, P7-L33-P2, P7-L33-P3, P7-L33-P4, P7-L33-P5, P7-L33-P6, P7-L33-P7, P7-L33-P8, P7-L34-P1, P7-L34-P2, P7-L34-P3, P7-L34-P4, P7-L34-P5, P7-L34-P6, P7-L34-P7, P7-L34-P8, P7-L35-P1, P7-L35-P2, P7-L35-P3, P7-L35-P4, P7-L35-P5, P7-L35-P6, P7-L35-P7, P7-L35-P8, P7-L36-P1, P7-L36-P2, P7-L36-P3, P7-L36-P4, P7-L36-P5, P7-L36-P6, P7-L36-P7, P7-L36-P8, P8-L1-P1, P8-L1-P2, P8-L1-P3, P8-L1-P4, P8-L1-P5, P8-L1-P6, P8-L1-P7, P8-L1-P8, P8-L2-P1, P8-L2-P2, P8-L2-P3, P8-L2-P4, P8-L2-P5, P8-L2-P6, P8-L2-P7, P8-L2-P8, P8-L3-P1, P8-L3-P2, P8-L3-P3, P8-L3-P4, P8-L3-P5, P8-L3-P6, P8-L3-P7, P8-L3-P8, P8-L4-P1, P8-L4-P2, P8-L4-P3, P8-L4-P4, P8-L4-P5, P8-L4-P6, P8-L4-P7, P8-L4-P8, P8-L5-P1, P8-L5-P2, P8-L5-P3, P8-L5-P4, P8-L5-P5, P8-L5-P6, P8-L5-P7, P8-L5-P8, P8-L6-P1, P8-L6-P2, P8-L6-P3, P8-L6-P4, P8-L6-P5, P8-L6-P6, P8-L6-P7, P8-L6-P8, P8-L7-P1, P8-L7-P2, P8-L7-P3, P8-L7-P4, P8-L7-P5, P8-L7-P6, P8-L7-P7, P8-L7-P8, P8-L8-P1, P8-L8-P2, P8-L8-P3, P8-L8-P4, P8-L8-P5, P8-L8-P6, P8-L8-P7, P8-L8-P8, P8-L9-P1, P8-L9-P2, P8-L9-P3, P8-L9-P4, P8-L9-P5, P8-L9-P6, P8-L9-P7, P8-L9-P8, P8-L10-P1, P8-L10-P2, P8-L10-P3, P8-L10-P4, P8-L10-P5, P8-L10-P6, P8-L10-P7, P8-L10-P8, P8-11-P1, P8-L11-P2, P8-L11-P3, P8-L11-P4, P8-L11-P5, P8-L11-P6, P8-L11-P7, P8-L11-P8, P8-L12-P1, P8-L12-P2, P8-L12-P3, P8-L12-P4, P8-L12-P5, P8-L12-P6, P8-L12-P7, P8-L12-P8, P8-L13-P1, P8-L13-P2, P8-L13-P3, P8-L13-P4, P8-L13-P5, P8-L13-P6, P8-L13-P7, P8-L13-P8, P8-L14-P1, P8-L14-P2, P8-L14-P3, P8-L14-P4, P8-L14-P5, P8-L14-P6, P8-L14-P7, P8-L14-P8, P8-L15-P1, P8-L15-P2, P8-L15-P3, P8-L15-P4, P8-L15-P5, P8-L15-P6, P8-L15-P7, P8-L15-P8, P8-L16-P1, P8-L16-P2, P8-L16-P3, P8-L16-P4, P8-L16-P5, P8-L16-P6, P8-L16-P7, P8-L16-P8, P8-L17-P1, P8-L17-P2, P8-L17-P3, P8-L17-P4, P8-L17-P5, P8-L17-P6, P8-L17-P7, P8-L17-P8, P8-L18-P1, P8-L18-P2, P8-L18-P3, P8-L18-P4, P8-L18-P5, P8-L18-P6, P8-L18-P7, P8-L18-P8, P8-L19-P1, P8-L19-P2, P8-L19-P3, P8-L19-P4, P8-L19-P5, P8-L19-P6, P8-L19-P7, P8-L19-P8, P8-L20-P1, P8-L20-P2, P8-L20-P3, P8-L20-P4, P8-L20-P5, P8-L20-P6, P8-L20-P7, P8-L20-P8, P8-L21-P1, P8-L21-P2, P8-L21-P3, P8-L21-P4, P8-L21-P5, P8-L21-P6, P8-L21-P7, P8-L21-P8, P8-L22-P1, P8-L22-P2, P8-L22-P3, P8-L22-P4, P8-L22-P5, P8-L22-P6, P8-L22-P7, P8-L22-P8, P8-L23-P1, P8-L23-P2, P8-L23-P3, P8-L23-P4, P8-L23-P5, P8-L23-P6, P8-L23-P7, P8-L23-P8, P8-L24-P1, P8-L24-P2, P8-L24-P3, P8-L24-P4, P8-L24-P5, P8-L24-P6, P8-L24-P7, P8-L24-P8, P8-L25-P1, P8-L25-P2, P8-L25-P3, P8-L25-P4, P8-L25-P5, P8-L25-P6, P8-L25-P7, P8-L25-P8, P8-L26-P1, P8-L26-P2, P8-L26-P3, P8-L26-P4, P8-L26-P5, P8-L26-P6, P8-L26-P7, P8-L26-P8, P8-L27-P1, P8-L27-P2, P8-L27-P3, P8-L27-P4, P8-L27-P5, P8-L27-P6, P8-L27-P7, P8-L27-P8, P8-L28-P1, P8-L28-P2, P8-L28-P3, P8-L28-P4, P8-L28-P5, P8-L28-P6, P8-L28-P7, P8-L28-P8, P8-L29-P1, P8-L29-P2, P8-L29-P3, P8-L29-P4, P8-L29-P5, P8-L29-P6, P8-L29-P7, P8-L29-P8, P8-L30-P1, P8-L30-P2, P8-L30-P3, P8-L30-P4, P8-L30-P5, P8-L30-P6, P8-L30-P7, P8-L30-P8, P8-L31-P1, P8-L31-P2, P8-L31-P3, P8-L31-P4, P8-L31-P5, P8-L31-P6, P8-L31-P7, P8-L31-P8, P8-L32-P1, P8-L32-P2, P8-L32-P3, P8-L32-P4, P8-L32-P5, P8-L32-P6, P8-L32-P7, P8-L32-P8, P8-L33-P1, P8-L33-P2, P8-L33-P3, P8-L33-P4, P8-L33-P5, P8-L33-P6, P8-L33-P7, P8-L33-P8, P8-L34-P1, P8-L34-P2, P8-L34-P3, P8-L34-P4, P8-L34-P5, P8-L34-P6, P8-L34-P7, P8-L34-P8, P8-L35-P1, P8-L35-P2, P8-L35-P3, P8-L35-P4, P8-L35-P5, P8-L35-P6, P8-L35-P7, P8-L35-P8, P8-L36-P1, P8-L36-P2, P8-L36-P3, P8-L36-P4, P8-L36-P5, P8-L36-P6, P8-L36-P7 and P8-L36-P8.

### Example B. Conjugation and Characterization of ADCs

### In Vitro Cysmab ADC Preparation

Antibody (typically 5-10 mg) was incubated with rProtein A Sepharose resin (GE) at a ratio of 10 mg Ab to 1 ml resin in PBS for 15 minutes with mixing in an appropriately sized disposable column. Cysteine HCl was added to a final concentration of 20 mM and incubated with agitation for 30 min at room temperature to allow the reactive cysteines to be deblocked. The resin was rapidly washed with 50 column volumes PBS on a vacuum manifold in multiple additions. The resin was then resuspended in an equal volume PBS containing 250 nM CuCl₂. Reformation of antibody interchain disulfides was monitored by taking time points. At each time point, 25 μL of resin slurry was removed, 1 μL of 20 mM MC-valcit-MMAE was added, and the tube flicked several times. The resin was spun down, supernatant removed, and then eluted with 50 μL Antibody elution buffer (Thermo). The resin was pelleted and the supernatant analyzed by reverse phase chromatography using an Agilent PLRP-S 4000A 5 um, 4.6×50 mm column (Buffer A is water, 0.1% TFA, Buffer B Acetonitrile, 0.1% TFA, column held at 80° C., Flowrate 1.5 ml/min; Gradient 0 minutes—30% B, 5 minutes—45% B, 6.5 min—100% B, 8 minutes—100% B, 10 minutes—30%).

Once determined that the antibody has reformed its interchain disulfide bonds, the resin was washed with 10 column volumes PBS and the resin was resuspended in an equal volume PBS and 12 equivalents of the appropriate linker-payload (20 mM) in DMSO was added and then incubated at room temperature for 2 hours. The resin was then washed with 50 column volumes PBS to remove excess linker-payload. The ADC was eluted from the protein A resin with antibody elution buffer. The ADC was then dialyzed into PBS. The material was then concentrated using a centrifugal concentrator using an Amicon Ultra-15, 50 KDa, regenerated cellulose (Millipore, UFC0905024), to 4.5 mg/ml and filtered sterilely through 0.22 μm sterile PVDF Filter, 25 mm (Millapore, SLGV013SL) and stored at 4° C. The following analyses were performed—analytical SEC to determine percent monomer, reduced mass spectroscopy to determine DAR, LAL test to determine endotoxin load and protein concentration was determined by A280 utilizing extinction coefficient and molecular weight of antibody. All in vitro materials were >90% monomer. Percent aggregation, as determined by comparison of the area of the high-molecular-weight peak absorbance at 210 and 280 nm with the area of the peak absorbance for monomeric ADC. HRMS data (protein method) indicated a dominant mass of the heavy chain+2 species, giving a DAR of ˜4.0 was calculated by comparing MS intensities of peaks for DAR1 DAR2 and DAR3 species.

General Methodology: Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1): 20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.1×150 mm, 8 um (Agilent, PL1912-3803). Flowrate used was 0.3 ml/min. The gradient used was 0-0.75 minute 95%A, 0.76-1.9 minute 75%A, 1.91-11.0 minute 50%A, 11.01-11.50 10% A, 11.51-13.50 minute 95%A, 13.51-18 minute 95%A on an Acuity Bio H-Class Quaternary UPLC (Waters). MS system was Xevo G2-XS QToF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.
Size exclusion chromatography (SEC): SEC was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

### In Vivo Cysmab ADC Preparation

Antibody (25-200 mg) was incubated with rProtein A Sepharose resin (Cytiva) at a ratio of 10 mg Ab to 1 ml resin in PBS for 15 minutes with mixing in an appropriately sized disposable column. Cysteine HCl was added to a final concentration of 20 mM and incubated with agitation for 30 min at room temperature to allow the reactive cysteines to be deblocked. The resin was rapidly washed with 50 column volumes phosphate buffered saline pH 7.2 (PBS) on a vacuum manifold in multiple additions. The resin was then resuspended in an equal volume PBS containing 250 nM CuCl₂. Reformation of antibody interchain disulfides was monitored by taking time points. At each time point, 25 μL of resin slurry was removed, 1 μL of 20 mM MC-valcit-MMAE was added, and the tube flicked several times. The resin was spun down, supernatant removed, and then eluted with 50 μL Antibody elution buffer (Thermo). The resin was pelleted and the supernatant analyzed by reverse phase chromatography using an Agilent PLRP-S 4000A Sum, 4.6×50 mm column (Buffer A is water, 0.1% TFA, Buffer B Acetonitrile, 0.1% TFA, column held at 80° C., Flowrate 1.5 ml/min; Gradient 0 minutes—30% B, 5 minutes—45% B, 6.5 min—100% B, 8 minutes—100% B, 10 minutes—30%).

Once it was determined that the antibody has reformed its interchain disulfide bonds, the resin was washed with 10 column volumes PBS and the resin was resuspended in an equal volume PBS and 12 equivalents of the appropriate linker-payload (20 mM) in DMSO was added and then incubated at room temperature for 2 hours. The resin was then washed with 50 column volumes PBS to remove excess linker-payload. The ADC was eluted from the protein A resin with antibody elution buffer. The ADC was then dialyzed into PBS and preparative SEC using a 16/60 or 26/600 S200 increase pg SEC column (GE) with PBS as the mobile phase if needed.

The material was then concentrated using a centrifugal concentrator using an Amicon Ultra-15, 50 KDa, regenerated cellulose (Millipore, UFC0905024), to 4.5 mg/ml and filtered sterilely through 0.22 μm sterile PVDF Filter, 25 mm (Millapore, SLGV013SL) and stored at 4° C. The following analyses were performed—analytical SEC to determine percent monomer, mass spectroscopy to determine DAR, LAL test to determine endotoxin load and protein concentration was determined by A280 utilizing extinction coefficient and molecular weight of antibody. All in vivo materials were >95% monomer. Aggregation was typically <10%. Percent aggregation, as determined by comparison of the area of the high-molecular-weight peak absorbance at 210 and 280 nm with the area of the peak absorbance for monomeric ADC. HRMS data (protein method) indicated a dominant mass of the heavy chain+2 species, giving a DAR of ˜4.0 was calculated by comparing MS intensities of peaks for DAR1 DAR2 and DAR3 species.

### General Methodology

Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1): 20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.1×150 mm, 8 um (Agilent, PL1912-3803). Flowrate used was 0.3 ml/min. The gradient used was 0-0.75 minute 95%A, 0.76-1.9 minute 75%A, 1.91-11.0 minute 50%A, 11.01-11.50 10%A, 11.51-13.50 minute 95%A, 13.51-18 minute 95%A on an Acuity Bio H-Class Quaternary UPLC (Waters). MS system was Xevo G2-XS QToF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.

Size exclusion chromatography (SEC): SEC was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

### Example C. In Vitro Assessment of EphA2 Dual ADCs

### Cell Lines

The EphA2 antibody drug conjugates were tested against nine endogenous cancer cell lines.

OCUM-1: Stomach/gastric cancer—JCRB No. JCRB0192 cultured in DMEM+0.5 mM sodium pyruvate+10% FBS
EBC-1: Lung cancer—JCRB No. JCRB0820 cultured in DMEM+10% FBS
SKCO-1: Colon cancer—ATCC No. HTB-39 cultured in Eagle's Minimum Essential Medium+10% FBS
The Her2 antibody drug conjugates were tested against nine endogenous cancer cell lines.
NCI-N87: Stomach/gastric cancer—ATCC No. CRL-5822 cultured in RPMI-1640+10% FBS
The PCAD antibody drug conjugates were tested against nine endogenous cancer cell lines.
BICR-22: Tongue cancer—ECACC No. 04072106 cultured in DMEM+2 mM Glutamine+2% FBS+0.4 micrograms/ml Hydrocortisone

### Inhibition of Cell Proliferation and Survival

The ability of the antibody drug conjugates to inhibit cell proliferation and survival was assessed using the Promega CellTiter-Glo® proliferation assay.

Cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cell viability and cell density were determined using a cell counter (Vi-Cell XR Cell Viability Analyzer, Beckman Coulter). Cells with higher than 85% viability were seeded in white clear bottom 384-well TC treated plates (Corning cat. #3765). Cells were seeded at a density of 1,000 cells per well in 45 μL of standard growth media. Plates were incubated at 5% CO₂, 37° C. overnight in a tissue culture incubator. The next day, free auristatin payloads and targeting ADCs were prepared at 10× in standard growth media. The prepared treatments were added to the cells resulting in final concentrations of 0.005-100 nM and a final volume of 50 μL per well. Each drug concentration was tested in quadruplets. Plates were incubated at 5% CO₂, 37° C. for 5 days in a tissue culture incubator, after which cell viability was assessed through the addition of 25 μL of CellTiter Glo® (Promega, cat #G7573), a reagent which lyses cells and measures total adenosine triphosphate (ATP) content. Plates were incubated at room temperature for 10 minutes to stabilize luminescent signals prior to reading using a luminescence reader (EnVision Multilabel Plate Reader, PerkinElmer). To evaluate the effect of the drug treatments, luminescent counts from wells containing untreated cells (100% viability) were used to normalize treated samples. A variable slope model was applied to fit a nonlinear regression curve to the data in GraphPad PRISM version 7.02 software. IC50 and Amax values were extrapolated from the resultant curves.

The concentrations of treatment required to inhibit 50% of cell growth or survival (IC50) were calculated with representative IC50 values of the cell lines tested summarized in Tables 1-4.

The representative cancer cell lines were shown to be sensitive to the permeable auristatin payloads with IC50s ranging from 0.23-1.38 nM. The ADCs targeting EphA2 had cytotoxic effect on cell proliferation on the representative cancer cell lines with IC50s ranging from 0.075-12.02 nM. The ADCs targeting Her2 had cytotoxic effect on cell proliferation on the representative cancer cell line with IC50s ranging from 0.047-0.117 nM. The ADCs targeting PCAD had cytotoxic effect on cell proliferation on the representative cancer cell line with IC50s ranging from 0.495-0.970 nM. These studies indicate that the auristatin ADCs were capable of inhibiting cell proliferation on various cancer cell lines expressing EphA2, Her2 and PCAD.

TABLE 1
EphA2 ADC Cytotoxicity

| | Cell Line | | | | | |
|---|---|---|---|---|---|---|
| Treatment | EBC1 | | OCUM1 | | SKCO1 | |
| (Antibody: | IC50 | Amax | IC50 | Amax | IC50 | Amax |
| Example No.) | (nM) | (Span) | (nM) | (Span) | (nM) | (Span) |
| 1C1: Example 1 | 0.409 | 87.7 | 12.02 | 51.1 | 10.66 | 119.5 |
| 1C1: Example 2 | 0.299 | 78.5 | 6.43 | 26.8 | 1.84 | 50.2 |
| 1C1: Example 3 | 0.075 | 78.2 | 1.39 | 49.9 | 0.111 | 80.6 |
| 1C1: Example 4 | 0.146 | 83.3 | 1.73 | 56.4 | 0.166 | 89.2 |

TABLE 2
Her2 ADC Cytotoxicity

| | |
|---|---|
| | Cell Line |
| Treatment | NCIN87 |

| (Antibody: Example No.) | IC50 (nM) | Amax (Span) |
|---|---|---|
| Trastuzumab: Example 1 | 0.076 | 90.7 |
| Trastuzumab: Example 2 | 0.117 | 79.2 |
| Trastuzumab: Example 3 | 0.047 | 66.8 |
| Trastuzumab: Example 4 | 0.057 | 67.1 |

TABLE 3
PCAD ADC Cytotoxicity

| | |
|---|---|
| | Cell Line |
| Treatment | BICR22 |

| (Antibody: Example No.) | IC50 (nM) | Amax (Span) |
|---|---|---|
| NOV169N31Q: Example 1 | 0.970 | 95.1 |
| NOV169N31Q: Example 2 | 0.921 | 95.7 |
| NOV169N31Q: Example 3 | 0.495 | 98.0 |
| NOV169N31Q: Example 4 | 0.685 | 99.5 |

TABLE 4
Unconjugated Drug Cytotoxicity

| | Cell Line | | | | | | | | | |
|---|---|---|---|---|---|---|---|---|---|---|
| | EBC1 | | OCUM1 | | NCIN87 | | SKCO1 | | BICR22 | |
| | IC50 | Amax | IC50 | Amax | IC50 | Amax | IC50 | Amax | IC50 | Amax |
| Treatment | (nM) | (Span) | (nM) | (Span) | (nM) | (Span) | (nM) | (Span) | (nM) | (Span) |
| Auristatin#1 | 0.230 | 77.0 | 1.38 | 72.3 | 1.20 | 75.7 | 1.13 | 81.6 | 0.271 | 89.7 |
| Auristatin#2 | — | — | — | — | — | — | — | — | — | — |

Auristatin#1 = 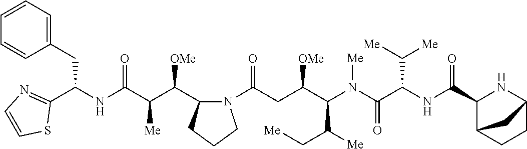 [SMILES: CCC(C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H]1N[C@@H]2CC[C@H]1C2)C(C)C]
Auristatin#2 =

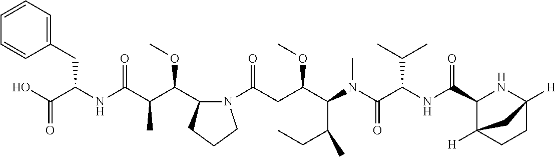 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H]1N[C@@H]2CC[C@H]1C2)C(C)C]

### Cell Lines

The Anti-HER2 TOPOi dual antibody drug conjugates Trastuzumab-Example 8 and Trastuzumab-Example 7 were tested against three breast cancer in vitro models.
BT474: ATCC No. HTB-20 cultured in Dulbecco's Modified Eagle Medium+10% FBS
UACC-812: ATCC No. CRL-1897 cultured in RPMI-1640+20% FBS+20 ng/mL EGF
ZR-75-30: ATCC No. CRL-1504 cultured in RPMI-1640+10% FBS

The antibody drug conjugates Trastuzumab-Example 5 and Trastuzumab-Example 6 were tested against three breast cancer in vitro models.
BT474: ATCC No. HTB-20 cultured in Dulbecco's Modified Eagle Medium+10% FBS
EFM-19: RRID: CVCL_0253 cultured in RPMI-1640+10% FBS
ZR-75-30: ATCC No. CRL-1504 cultured in RPMI-1640+10% FBS

### Inhibition of Cell Proliferation and Survival

The ability of Trastuzumab-Example 8, Trastuzumab-Example 7, Trastuzumab-Example 5 and Trastuzumab-Example 6 to inhibit cell proliferation and survival was assessed using the Promega CellTiter-Glo® proliferation assay.

Cell lines were cultured in media that is optimal for their growth at 5% CO₂ and 37° C. in a tissue culture incubator. Prior to seeding for the proliferation assay, the cells were split at least 2 days before the assay to ensure optimal growth density. On the day of seeding, adherent cells were lifted off tissue culture flasks using Trypsin-EDTA (0.05%) (Thermo Fisher cat. #25300054). Cell viability and cell density were determined using a cell counter (Vi-Cell BLU Cell Viability Analyzer, Beckman Coulter). Cells with higher than 85% viability were seeded in white clear bottom 384-well TC treated plates (Corning cat. #3765). Cells were seeded at a density of 1,000 cells per well in 45 μL of standard growth media, with the exception of ZR-75-30 and UACC-812 which were seeded at a density of 2,000 cells per well in 45 μL of standard growth media. Plates were incubated at 5% CO₂ and 37° C. overnight in a tissue culture incubator.

On the day of dosing, the HER2 targeting ADCs and HER2 were prepared at 10× in standard growth media. The prepared targeting ADC treatments were added to the cells resulting in final concentrations of 3.81e⁻⁵ nM to 100 nM and a final volume of 50 uL per well. Each drug concentration was tested in quadruplets. The same day, cell viability of untreated cells at time of dosing was assessed through the addition of 25 μL of CellTiter Glo® (Promega, cat #G7573), a reagent which lyses cells and measures total adenosine triphosphate (ATP) content. Plates were incubated at room temperature for 10 minutes to stabilize the luminescent signals prior to reading. The luminescent signal for all plates was measured using a luminescence reader (PHERAstar FSX Plate Reader, BMG Labtech). This signal represents the cell viability of untreated cells on the day of dosing, or day 0.

Plates were incubated at 5% CO₂ and 37° C. for 5 days in a tissue culture incubator, after which cell viability was assessed through the addition of 25 μL of CellTiter Glo® (Promega, cat #G7573). Plates were incubated at room temperature for 10 minutes to stabilize luminescent signals. All assay plates were read using a luminescence reader (PHERAstar FSX Plate Reader, BMG Labtech). To evaluate the effect of the drug treatments, luminescent counts from wells containing untreated cells (100% viability) at day 5 and day 0 were used to normalize treated samples. A variable slope model was applied to fit a nonlinear regression curve to the data in GraphPad PRISM version 9.4.1 software. IC50 and Amax/Span values were extrapolated from the resultant curves.

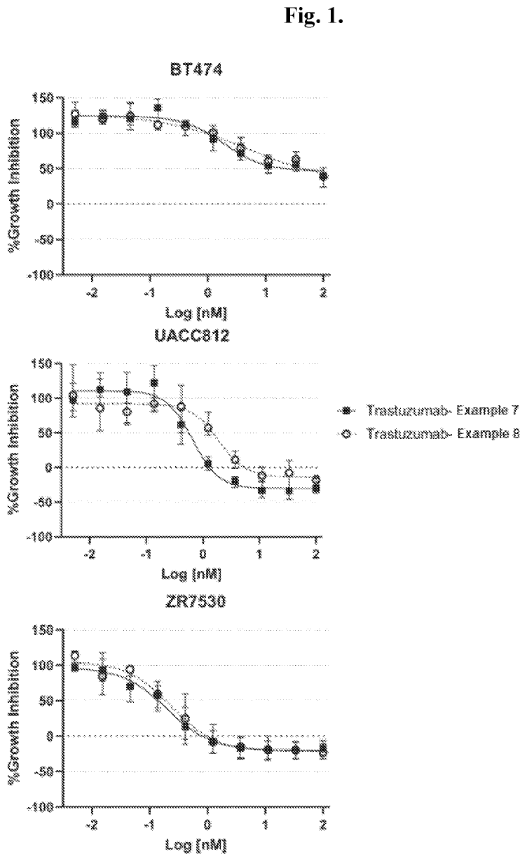

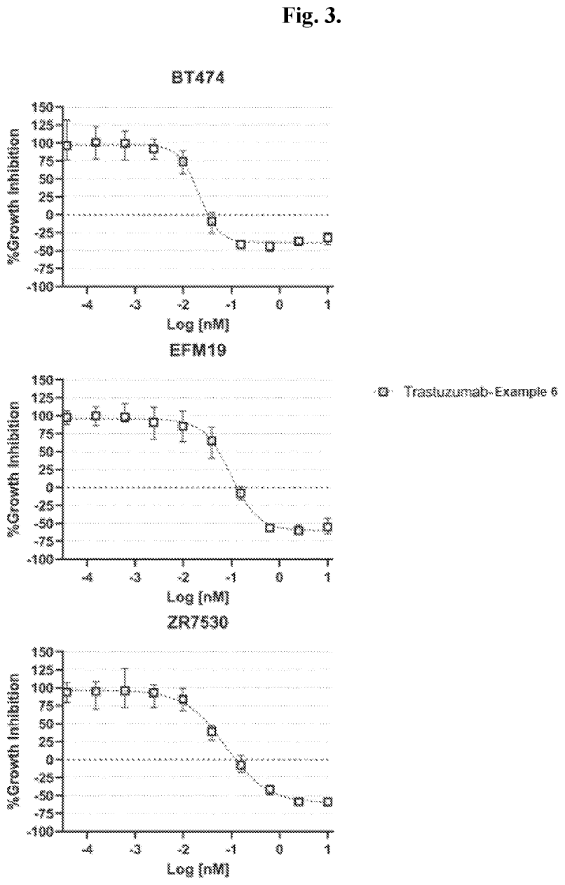

The dose response curves of Trastuzumab-Example 8 (FIG. 1), Trastuzumab-Example 7 (FIG. 1), Trastuzumab-Example 5 (FIG. 12, and Trastuzumab-Example 6 (FIG. 3) against representative cancer cell lines are shown in the indicated Figures. The concentrations of treatment required to inhibit 50% of cell growth or survival (IC50) were calculated with representative IC50 values of the cell lines tested summarized in Tables 5, 6, and 7 respectively.

The representative breast cancer cell lines were shown to be sensitive to the Dual ADCs with IC50 values ranging from 0.01 nM to 5 nM. These studies indicate that HER2 targeting Dual ADCs with multiple payload formats were capable of inhibiting cell proliferation on various breast cancer cell lines expressing HER2.

TABLE 5
Anti-HER2 TOPOi dual ADC Cytotoxicity (IC50) on Breast Models
IC50 (nM) of Compounds on Breast Models

| Compound | BT474 | UACC812 | ZR7530 |
|---|---|---|---|
| Trastuzumab- | 4.991 | 1.892 | 0.239 |
| Example 8 | | | |
| Trastuzumab- | 1.888 | 0.6574 | 0.1996 |
| Example 7 | | | |

TABLE 6
Anti-HER2 AuriX dual ADC
Cytotoxicity (IC50) on Breast Models
IC50 (nM) of Compounds on Breast Models

| Compound | BT474 | EFM19 | ZR7530 |
|---|---|---|---|
| Trastuzumab- | 0.01165 | 0.04618 | 0.04080 |
| Example 5 | | | |

TABLE 7
Anti-HER2 AuriX/TOPOi dual ADC
Cytotoxicity (IC50) on Breast Models
IC50 (nM) of Compounds on Breast Models

| Compound | BT474 | EFM19 | ZR7530 |
|---|---|---|---|
| Trastuzumab- | 0.02080 | 0.09663 | 0.07600 |
| Example 6 | | | |

1-257. (canceled)

258. A compound or pharmaceutically acceptable salt thereof, of formula (C-1): 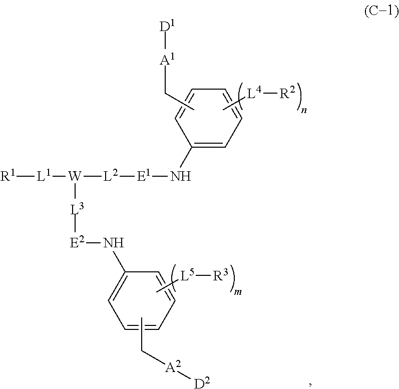
wherein:
R¹ is an attachment group;
L¹ is a bridging spacer;
W is N or CR^{w}, wherein R^{w} is H or C₁-C₆alkyl (e.g. R^{w} is H);
L² and L³ are each independently a connecting spacer;
E¹ and E² are each independently a peptide group comprising 1 to 6 amino acids, wherein said peptide group is optionally substituted by a hydrophilic group;
A¹ and A² are each independently a bond, —OC(═O)—*, 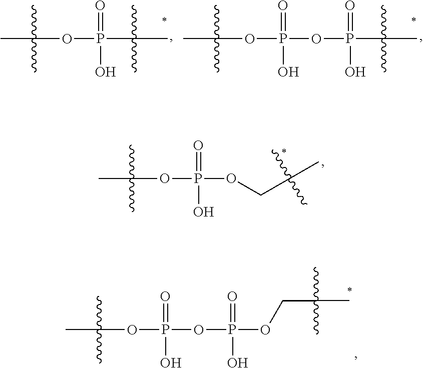 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆alkyl, and C₃-C₈cycloalkyl and the * of A¹ or A² indicates the point of attachment to D¹ or D²;
D¹ and D² are each independently a pharmaceutically active drug, optionally with the proviso that neither D¹ nor D² is a BH3 mimetic;
L⁴ and L⁵ are each independently a spacer moiety;
R² and R³ are each independently selected from:
(i) a polysarcosine with the following moiety: 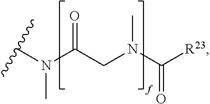
wherein
f is an integer between 3 and 25; and
R²³ is H, —CH₃ or —CH₂CH₂C(═O)OH;
(ii) a polyethylene glycol of formula: 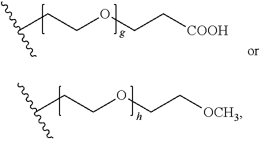
wherein g and h are independently an integer between 2 and 30; and
m and n are each independently 1;
or a pharmaceutically acceptable salt thereof.

259. The compound or pharmaceutically acceptable salt thereof according to claim 258, of formula (D1-1)

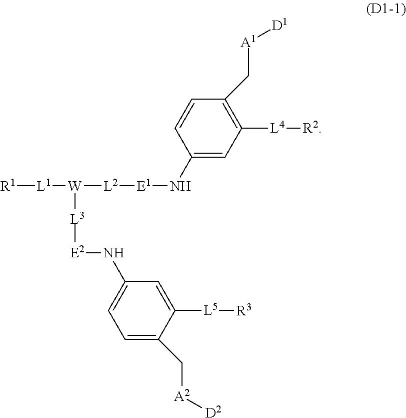

260. The compound or pharmaceutically acceptable salt thereof according to claim 258, wherein R¹ is selected from the group consisting of: 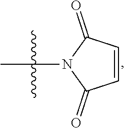 [SMILES: *N1C(=O)C=CC1=O]
—ONH₂, —NH₂, 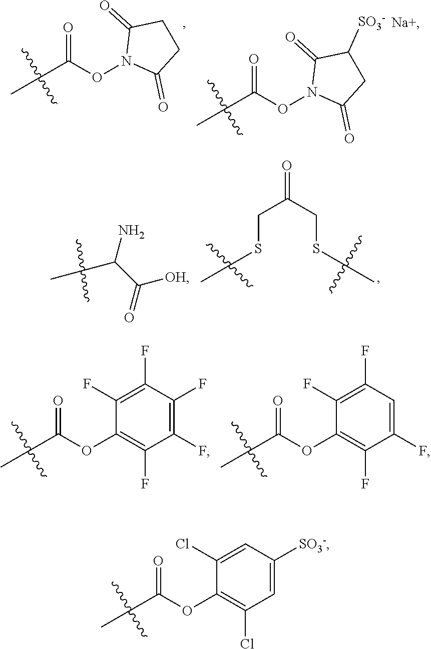
—N₃, 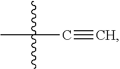 [SMILES: *C#C]
—SH, —SR¹¹, —SSR¹², —S(═O)₂(CH═CH₂), —(CH₂)₂S(═O)₂(CH═CH₂), —NHS(═O)₂(CH═CH₂), —NR¹¹C(═O)CH₂Br e.g. —NHC(═O)CH)C, —CH₂O(═O)CH₂I, e.g. —NHC(═O)CH₂I, 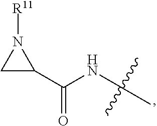
—C(O)NHNH₂, 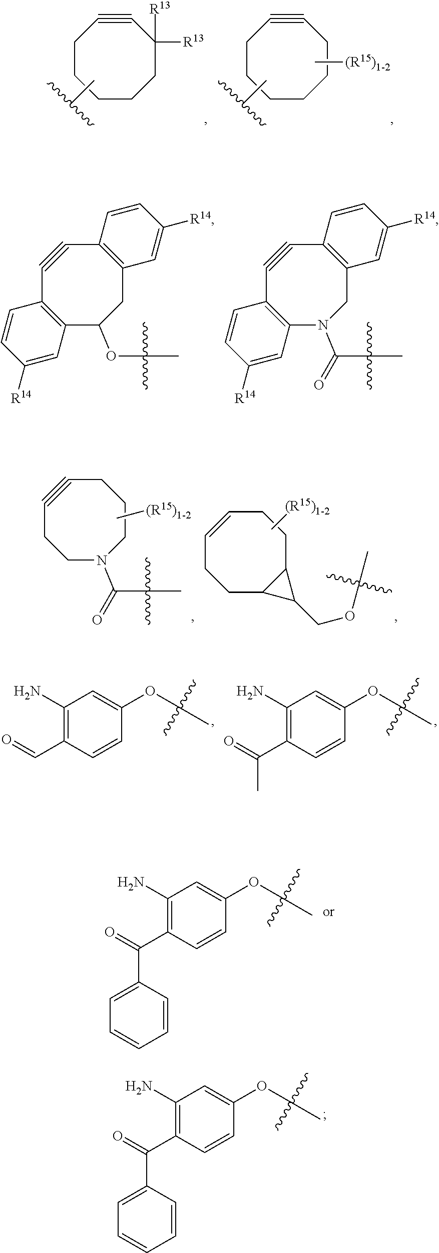
wherein:
each R¹¹ is independently selected from H and C₁-C₆alkyl;
each R¹² is 2-pyridyl or 4-pyridyl;
each R¹³ is independently selected from H, C₁-C₆alkyl, F, Cl, and —OH;
each R¹⁴ is independently selected from H, C₁-C₆alkyl, F, Cl, —NH₂, —OCH₃, —OCH₂CH₃, —N(CH₃)₂, —CN, —NO₂ and —OH; and
each R¹⁵ is independently selected from H, C₁-C₆alkyl, fluoro, benzyloxy substituted with —C(═O)OH, benzyl substituted with —C(═O)OH, C₁-C₄alkoxy substituted with —C(═O)OH and C₁-C₄alkyl substituted with —C(═O)OH.

261. The compound or pharmaceutically acceptable salt thereof according to claim 260, wherein R¹ is selected from the group consisting of: 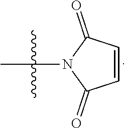 [SMILES: *N1C(=O)C=CC1=O]
—ONH₂, 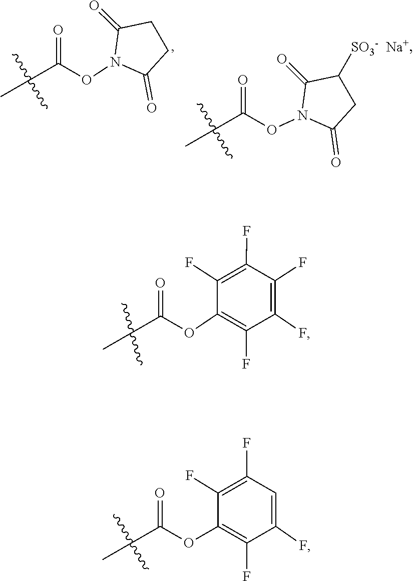
NHC(═O)CH₂Br and —NHC(═O)CH₂I.

262. The compound or pharmaceutically acceptable salt thereof according to claim 258, wherein:
(1) L¹ comprises: 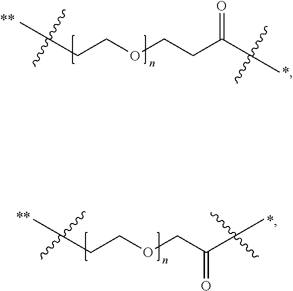
or *—CH(OH)CH(OH)CH(OH)CH(OH)—**,
wherein each n is an integer from 1 to 12, wherein the * of L¹ indicates the point of direct or indirect (e.g. direct) attachment to W, and the ** of L¹ indicates the point of direct or indirect (e.g. direct) attachment to R¹;
(2) L¹ is 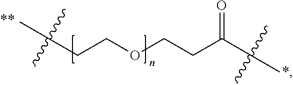
and n is an integer from 1 to 12 or n is 1 or n is 12, wherein the * of L¹ indicates the point of direct or indirect (e.g. direct) attachment to W, and the ** of L¹ indicates the point of direct or indirect (e.g. direct) attachment to R¹;
(3) L¹ is 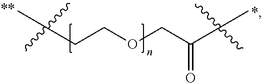
and n is an integer from 1 to 12, wherein the * of L¹ indicates the point of direct or indirect (e.g. direct) attachment to W, and the ** of L₁ indicates the point of direct or indirect (e.g. direct) attachment to R¹;
(4) L¹ comprises 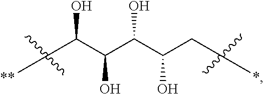 [SMILES: *C[C@H](O)[C@@H](O)[C@@H](O)[C@@H](*)O]
wherein the * of L¹ indicates the point of direct or indirect (e.g. direct) attachment to W, and the ** of L¹ indicates the point of direct or indirect (e.g. direct) attachment to R¹; or
(5) L¹ is a bridging spacer comprising:
*—C(═O)(CH₂)ₘO(CH₂)ₘ—**; *—C(═O)((CH₂)ₘO)ₜ(CH₂)ₙ—**; *—C(═O)(CH₂)ₘ—**;
*—C(═O)N H((CH₂)ₘO)ₜ(CH₂)ₙ—**;
*—C(═O)O(CH₂)ₘSSC(R^{L1})₂(CH₂)ₘC(═O)NR^{L1}(CH₂)ₘNR^{L1}C(═O)(CH₂)ₘ—**;
*—C(═O)O(CH₂)ₘC(═O)NH(CH₂)ₘ—**; *—C(═O)(CH₂)ₘNH(CH₂)ₘ—**;
*—C(═O)(CH₂)ₘNH(CH₂)ₙC(═O)—**; *—C(═O)(CH₂)ₘX₁(CH₂)ₘ—**;
*—C(═O)((CH₂)ₘO)ₜ(CH₂)ₙX₁(CH₂)ₙ—**; *—C(═O)(CH₂)ₘNHC(═O)(CH₂)ₙ—**;
*—C(═O)((CH₂)ₘO)ₜ(CH₂)ₙNHC(═O)(CH₂)ₙ—**;
*—C(═O)(CH₂)ₘNHC(═O)(CH₂)ₙX₁(CH₂)ₙ—**;
*—C(═O)((CH₂)ₘO)ₜ(CH₂)ₙNHC(═O)(CH₂)ₙX₁(CH₂)ₙ—**;
*—C(═O)((CH₂)ₘO)ₜ(CH₂)ₙC(═O)NH(CH₂)ₘ—**; *—C(═O)(CH₂)ₘC(R^{L1})₂—** or
*—C(═O)(CH₂)ₘC(═O)NH(CH₂)ₘ—**, wherein the * of L¹ indicates the point of direct or indirect (e.g. direct) attachment to W, and the ** of L¹ indicates the point of direct or indirect (e.g. direct) attachment to R¹;
X₁ is 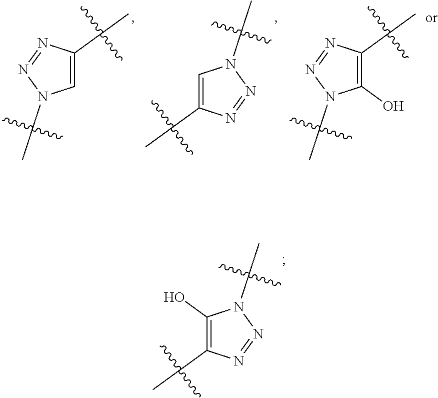 [SMILES: *c1cn(*)nn1 ; *c1cn(*)nn1 ; *c1nnn(*)c1O ; *c1nnn(*)c1O]
and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27,28,29 and 30; and
each R^{L1} is independently selected from H and C₁-C₆alkyl.

263. The compound or pharmaceutically acceptable salt thereof according to claim 262, wherein L¹ comprises a moiety represented by 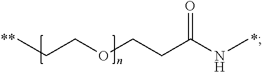
wherein n is an integer from 1 to 12 (e.g. 4 to 8, e.g. 4 or 8), wherein the * of L¹ indicates the point of direct or indirect (e.g. direct) attachment to W, and the ** of L¹ indicates the point of direct or indirect attachment to R¹.

264. The compound or pharmaceutically acceptable salt thereof according to claim 258, wherein L¹ is represented by a formula 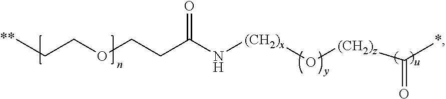
wherein
n is an integer from 1 to 12 (e.g. 4 to 8, e.g. 4 or 8);
x is an integer from 0 to 6 (e.g. 0 to 4, e.g. 0 to 2, e.g. 0 or 2);
y is 0 or 1;
z is an integer from 0 to 6 (e.g. 0 to 4, e.g. 0 to 2, e.g. 0 or 2);
u is 0 or 1; and
wherein the * of L¹ indicates the point of direct attachment to W, and the ** of L¹ indicates the point of direct attachment to R¹.

265. The compound or pharmaceutically acceptable salt thereof according to claim 258, wherein L¹ is selected from the group consisting of:

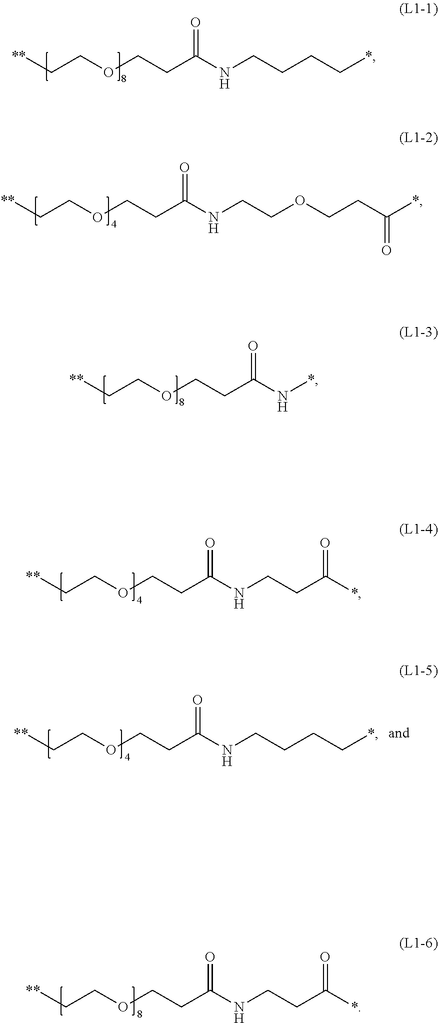

266. The compound or pharmaceutically acceptable salt thereof according to claim 258, wherein L² and L³ are each independently a connecting spacer comprising a moiety represented by: 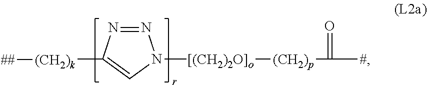
wherein
k is an integer from 0 to 6;
r is 0 or 1;
o is an integer from 0 to 12;
p is an integer from 0 to 6; and
wherein the # of L² or L³ indicates the point of direct or indirect attachment to E¹ or E², respectively, and the ## of L² or L³ indicates the point of direct or indirect attachment to W.

267. The compound or pharmaceutically acceptable salt thereof according to claim 258, wherein
L² and L³ are each independently a connecting spacer selected from a group consisting of 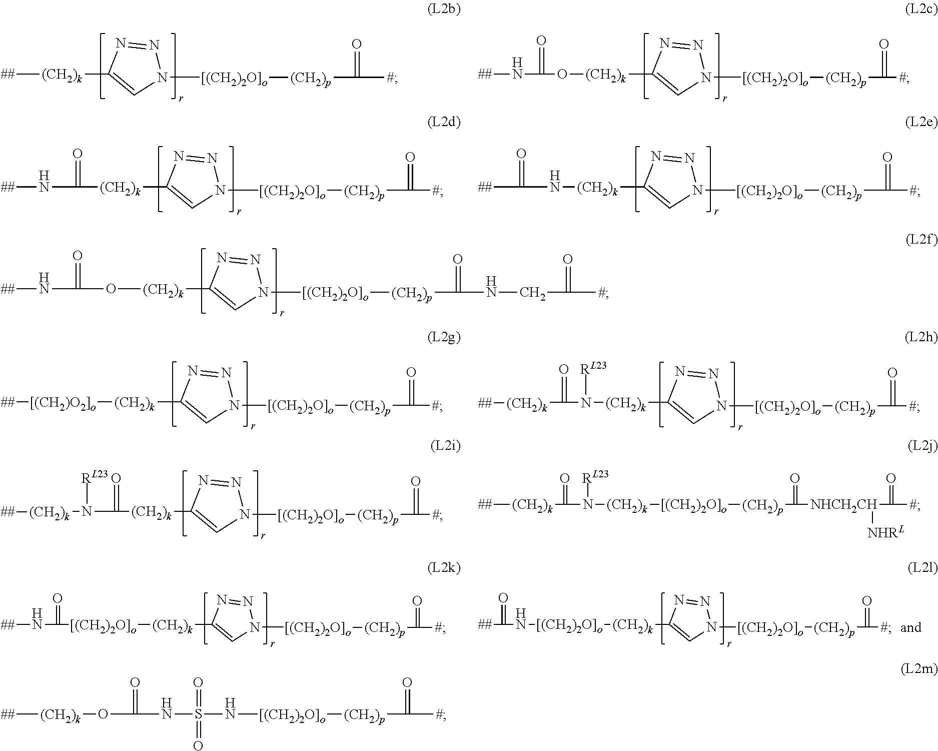
wherein
k, in each occurrence, is independently an integer from 0 to 4;
r, in each occurrence, is independently 0 or 1;
o, in each occurrence, is independently an integer from 0 to 10;
p, in each occurrence, is independently an integer from 0 to 4;
R^{L23} is hydrogen or C₁-C₆alkyl;
R^{L} is hydrogen or —C(O)—R^{H};
R^{H} is a hydrophilic group; and
the # of L² or L³ indicates the point of direct attachment to E¹ or E², respectively, and the ## of L² or L³ indicates the point of direct attachment to W;
provided that when W is N, L² and L³ are not (L2c), (L2d), (L2f) or (L2k),

268. The compound or pharmaceutically acceptable salt thereof according to claim 267, wherein L² and L³ are each independently a connecting spacer selected from a group consisting of: 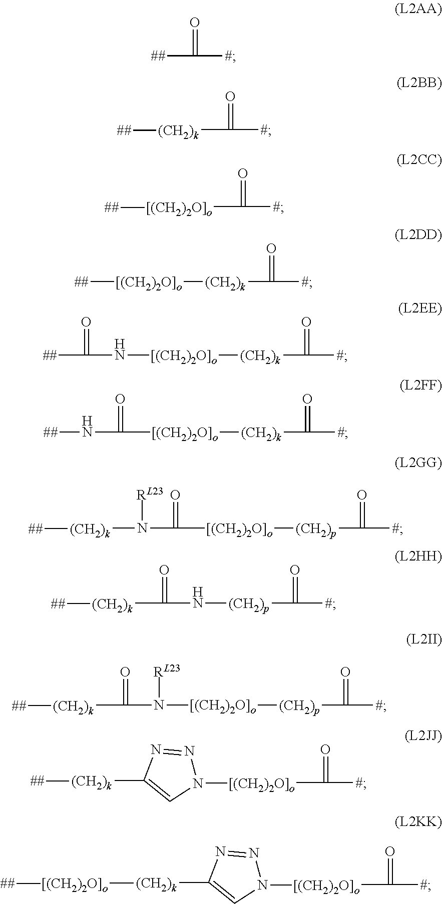 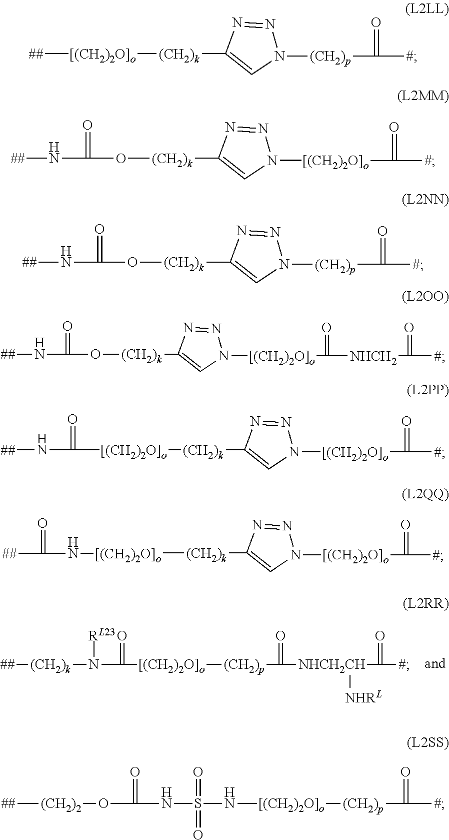
wherein
k, in each occurrence, is independently an integer from 1 to 3;
o, in each occurrence, is independently an integer from 1 to 9;
p, in each occurrence, is independently an integer from 1 to 3;
R^{L23} is hydrogen or C₁-C₃alkyl;
R^{L} is hydrogen or —C(O)—R^{H};
R^{H} is a hydrophilic group; and
the # of L² or L³ indicates the point of direct attachment to E¹ or E², respectively, and the ## of L² or L³ indicates the point of direct attachment to W; provided that when W is N, L² and L³ are not (L2FF), (L2MM), (L2NN), (L200), or (L2PP).

269. The compound or pharmaceutically acceptable salt thereof according claim 258, wherein L² and L³, independently, are a connecting spacer selected from a group consisting of: 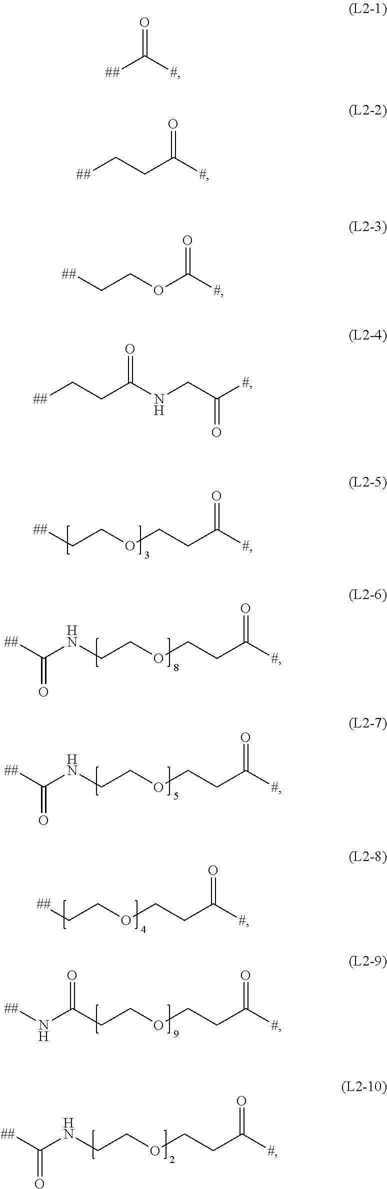 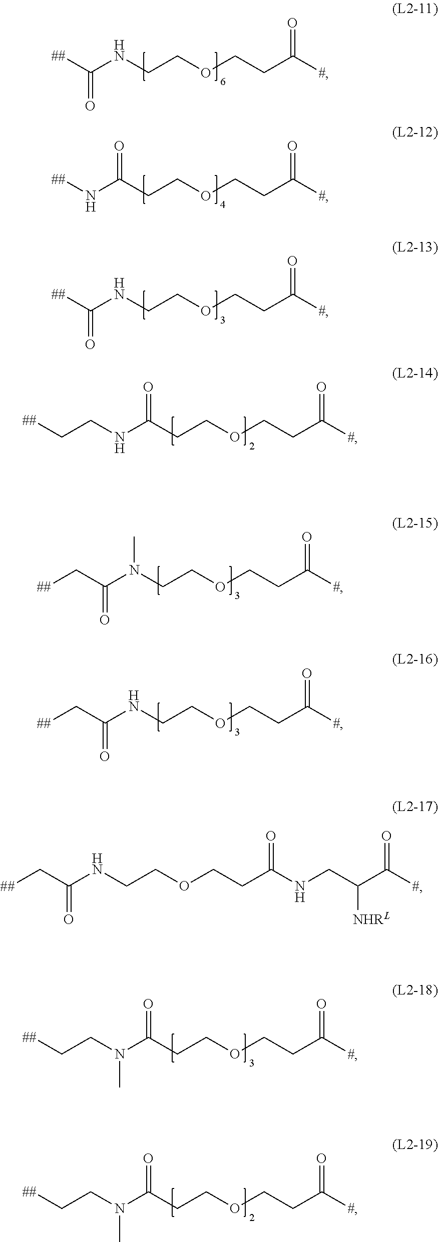 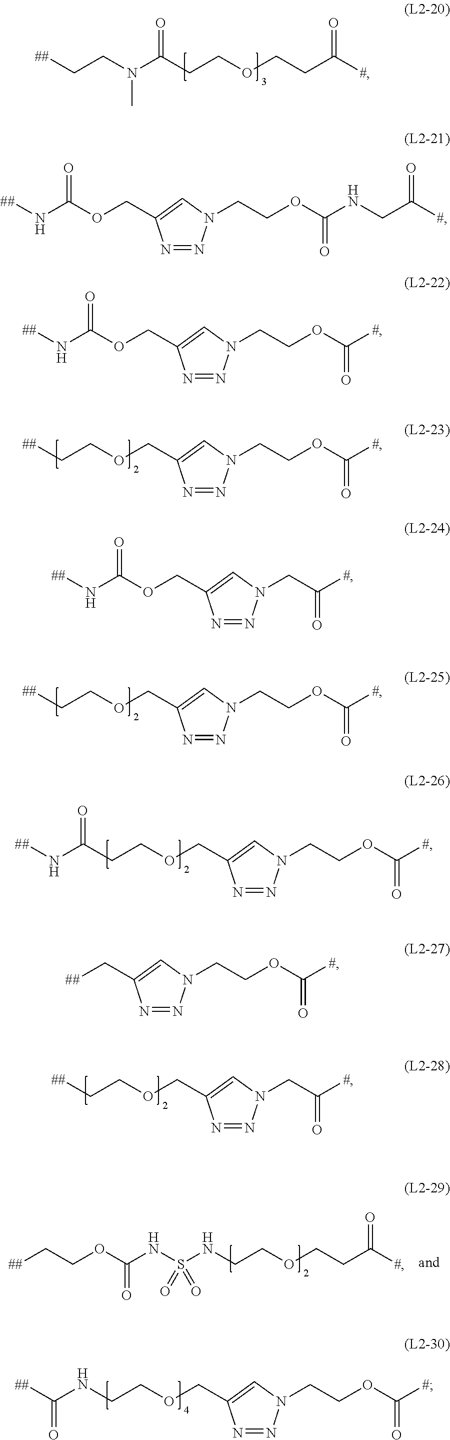
wherein the # of L² or L³ indicates the point of direct attachment to E¹ or E², respectively, the ## of L² or L³ indicates the point of direct attachment to W; R^{L} is hydrogen or —C(O)—R^{H}; and
R^{H} is 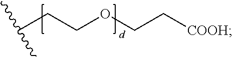
and d is an integer from 20 to 30 (e.g. 25).

270. The compound or pharmaceutically acceptable salt thereof according to claim 258, wherein each peptide group independently comprises 1 to 4, 1 to 3, or 1 to 2 amino acid residues, wherein each amino acid residues is independently selected from glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), L-tyrosine (Tyr) and β-alanine (β-Ala).

271. The compound or pharmaceutically acceptable salt according to claim 258, wherein each peptide group is independently selected from Val-Cit, Phe-Lys, Val-Ala, Val-Lys, Leu-Cit, Cit-(β-Ala), Gly-Gly-Gly, Gly-Gly-Phe-Gly, and sulfo-Ala-Val-Ala.

272. The compound or pharmaceutically acceptable salt according to claim 258, wherein E¹ and/or E², independently, is/are each a peptide group selected from a group consisting of: 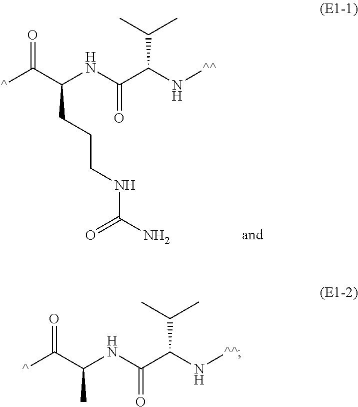
wherein \hat{ } of E1-1 or E1-2 indicates the point of direct attachment to V¹ or V² in Formula (B) or direct attachment to the —NH— group in Formula (C) and (D); and \hat{ }\hat{ } of E1-1 or E1-2 indicates the point of direct attachment to L² or L³, respectively (e.g. where E¹ and E² are each a peptide group independently selected from (E1-1) and (E1-2)).

273. The compound or pharmaceutically acceptable salt thereof according to claim 258, wherein E¹ and/or E², independently, is/are each a peptide group represented by 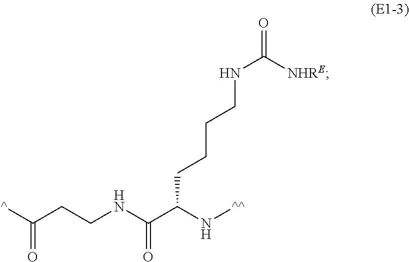
wherein R^{E} is a hydrophilic group R^{H}.

274. The compound or pharmaceutically acceptable salt thereof according to claim 273, wherein the hydrophilic group R^{H} in (E1-3) is 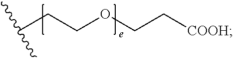
wherein e is an integer between 20 and 30 (e.g. 25).

275. The compound or pharmaceutically acceptable salt according to claim 258, wherein A¹ and A² are independently selected from a bond, 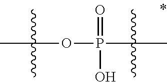 [SMILES: *OP(*)(=O)O]
and —OC(═O)—*, wherein * indicates the point of attachment to D¹ or D² respectively.

276. The compound or pharmaceutically acceptable salt according to claim 258, wherein:
i) L⁴ and L⁵ are each independently a spacer moiety having the structure 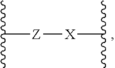
wherein:
Z is —O—, —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{L45}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is a bond, triazolyl, or —CH₂—triazolyl-,
wherein X is connected to R² or R³; or
(ii) L⁴ and L⁵, independently, are a spacer moiety having the structure 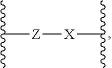
wherein:
Z is —CH₂—, —CH₂O—, —CH₂N(R^{L45})C(═O)O—, —NHC(═O)C(R^{L45})₂NHC(═O)O—, —NHC(═O)C(R^{L45})₂NH—, —NHC(═O)C(R^{L45})₂NHC(═O)—, —C(═O)NR^{b}—, —C(═O)NH—, —CH₂NR^{L45}C(═O)—, —CH₂NR^{L45}C(═O)NH—, —CH₂NR^{L45}C(═O)NR^{L45}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{L45} is independently selected from H, C₁-C₆alkyl, and C₃-C₈cycloalkyl; and
X is —CH₂—triazolyl-C₁-C₄alkylene-OC(O)NHS(O)₂NH—, —C₄-C₆cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂—triazolyl-C₁-C₄alkylene-OC(O)NHS(O)₂NH—(CH₂CH₂O)ₙ—, —C₄-C₆cycloalkylene-OC(O)NHS(O)₂NH—(CH₂CH₂O)ₙ—, wherein each n independently is 1, 2, or 3,
wherein X is connected to R² or R³,

277. The compound or pharmaceutically acceptable salt thereof according to claim 276, wherein L⁴ and L⁵ are each independently a spacer moiety selected from a group consisting of 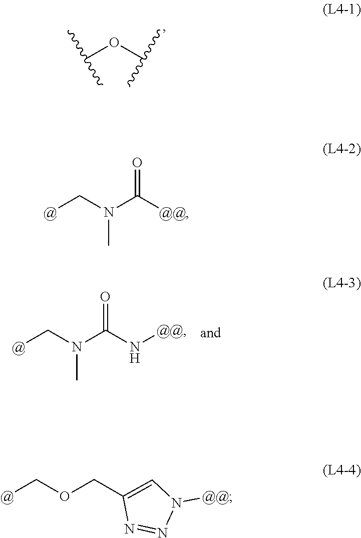
wherein the @ of L⁴ or L⁵ indicates the point of direct attachment to the phenyl group, and the @@ of L⁴ or L⁵ indicates the point of direct attachment to R² or R³.

278. The compound or pharmaceutically acceptable salt thereof according to claim 258, wherein R² and R³ each independently comprises a polyethylene glycol of formula: 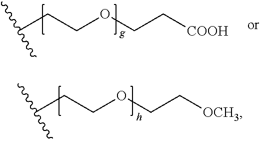
wherein g and h are independently an integer between 2 and 30.

279. A conjugate comprising an antibody or an antigen-binding fragment thereof covalently linked to two pharmaceutically active drugs through a dual linker, wherein the dual linker has one attachment point connected to the antibody and two attachment points to the two pharmaceutically active drugs, with the proviso that neither pharmaceutically active drug is a BH3 mimetic, and wherein the conjugate comprises the compound or pharmaceutically acceptable salt thereof according to claim 258.

280. A composition comprising multiple copies of the antibody-drug conjugate according to claim 279, wherein the average a of the antibody-drug conjugates in the composition is from about 1 to about 8, e.g., about 1 to about 6, about 1 to about 4, or about 1 to about 2.

281. The compound or pharmaceutically acceptable salt thereof according to claim 258, represented by any one of the following formulae: 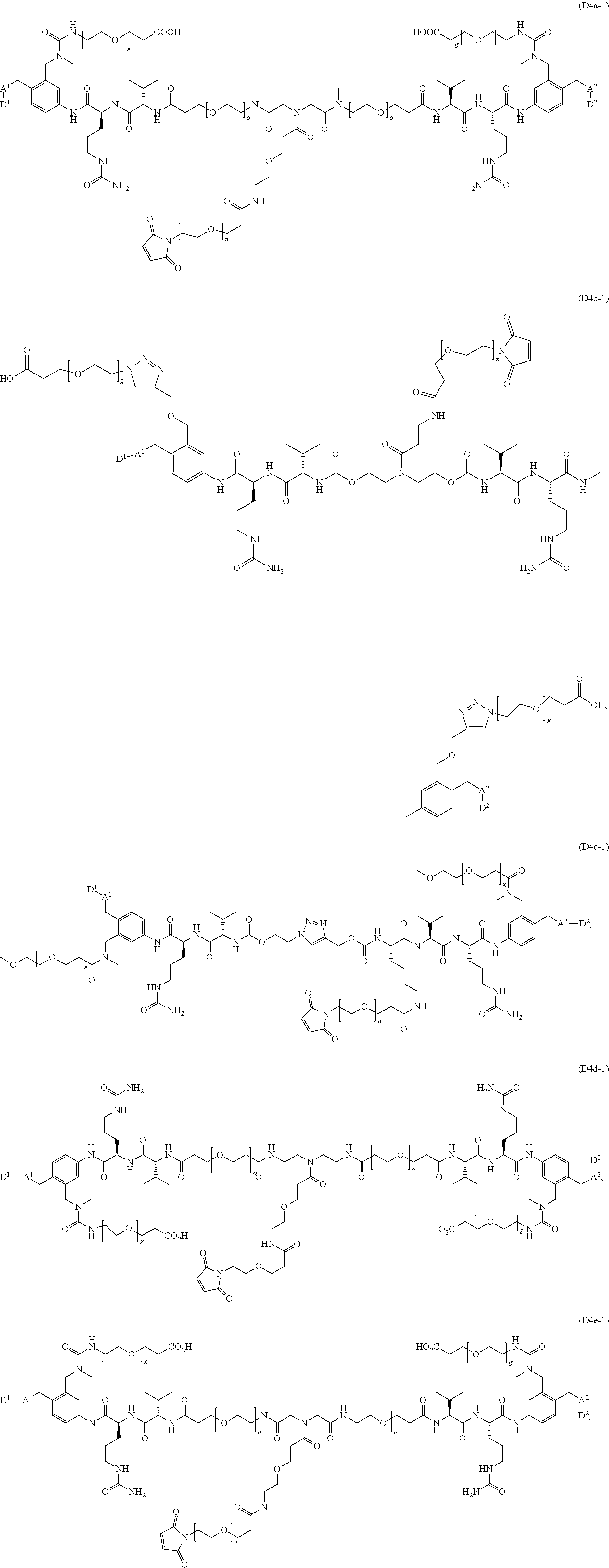 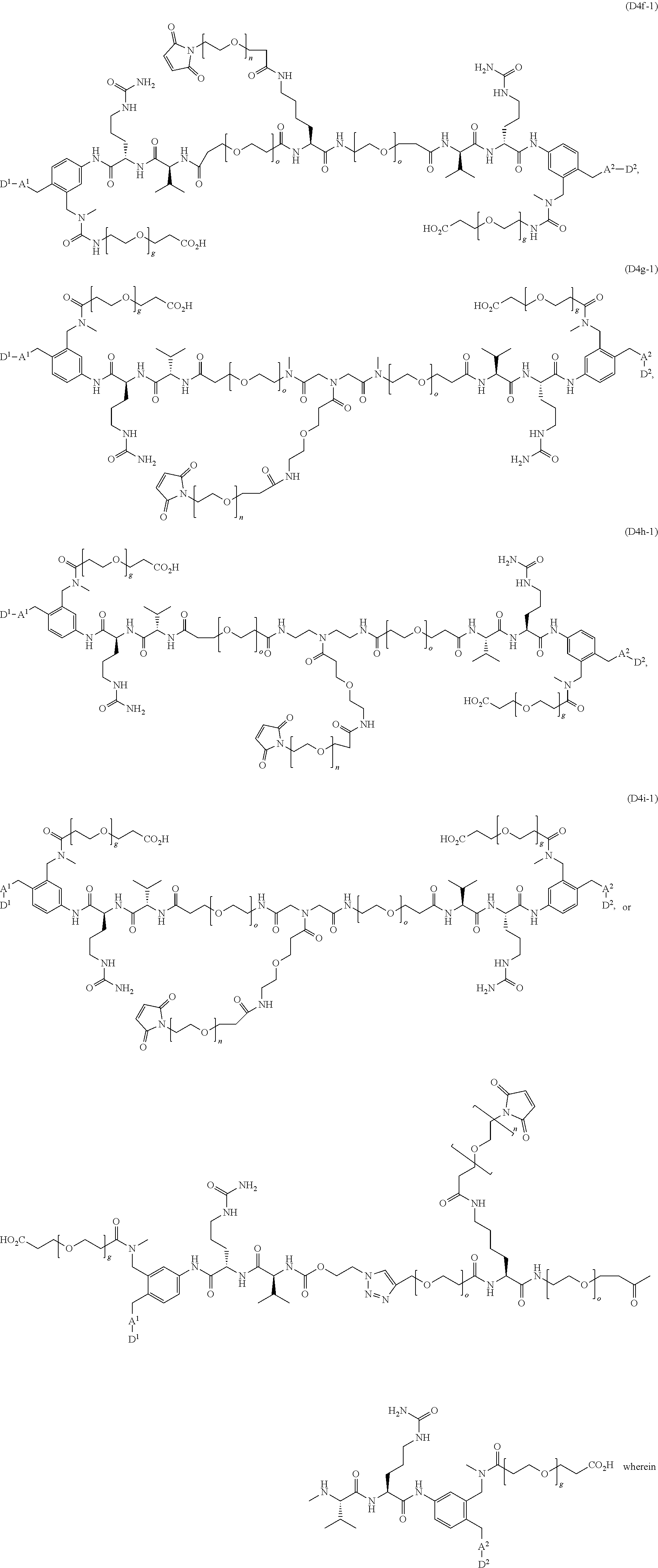
A¹ and A² are each independently selected from a bond, 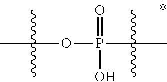 [SMILES: *OP(*)(=O)O]
and —O—C(═O)—*, wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 2 and 5); and
n is an integer between 1 and 12 (e.g., between 2 and 5).

282. The conjugate according to claim 279, represented by any one of the following formulae: 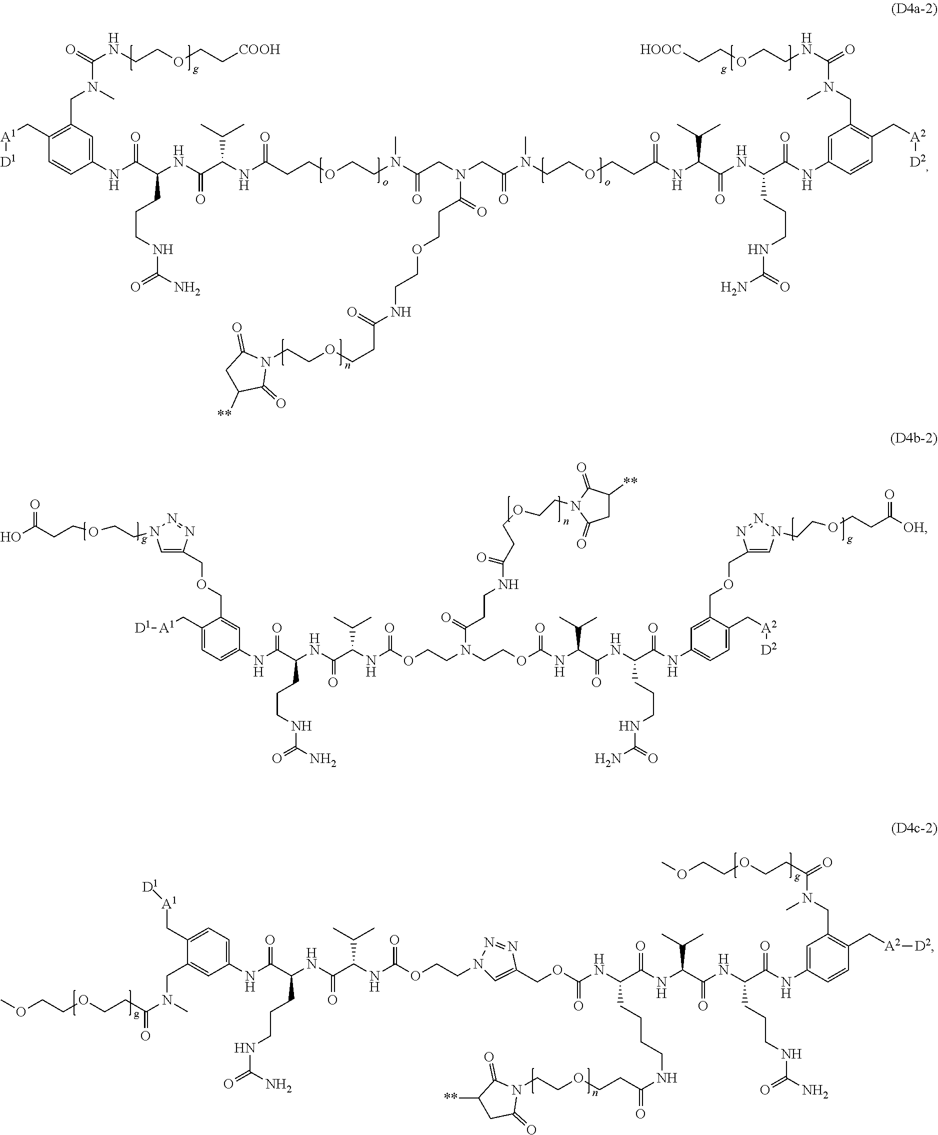 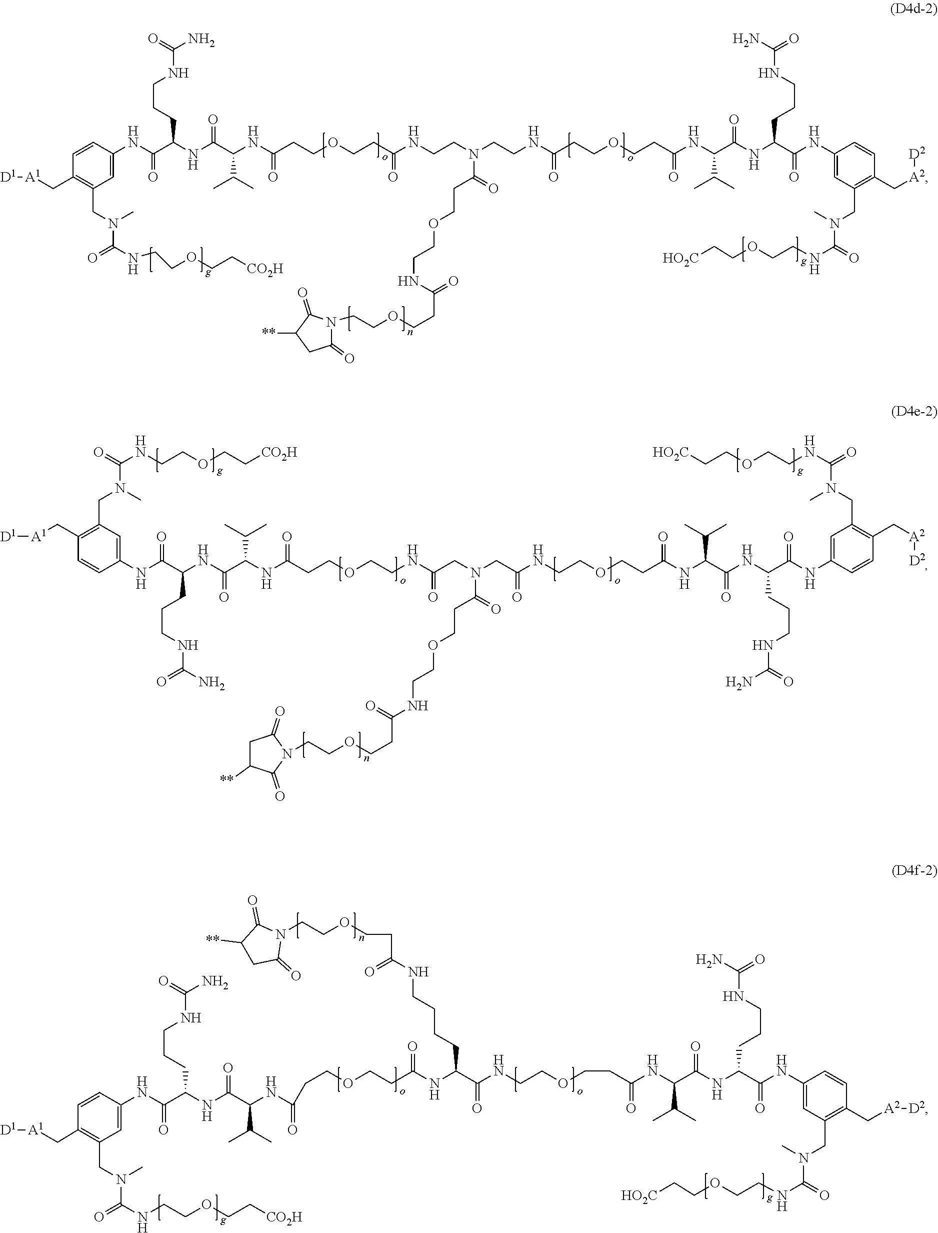 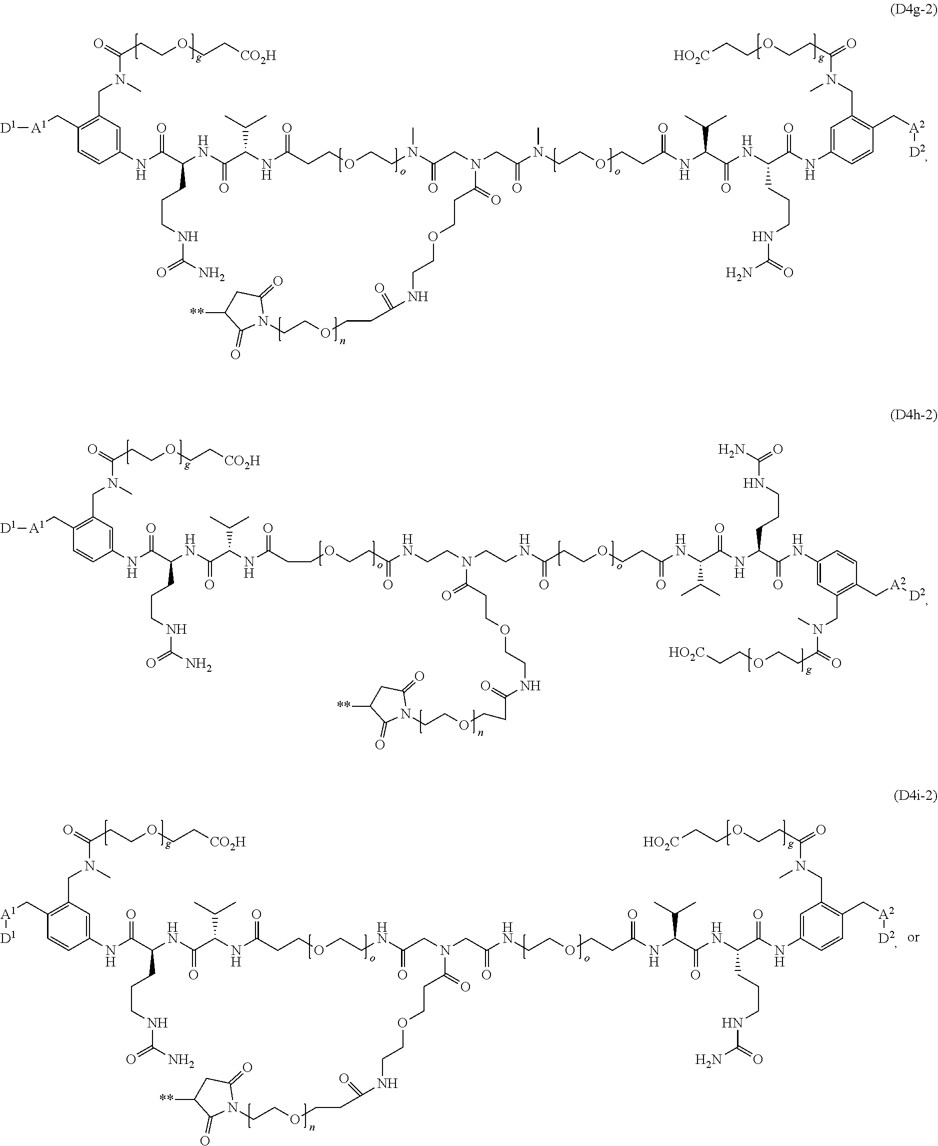 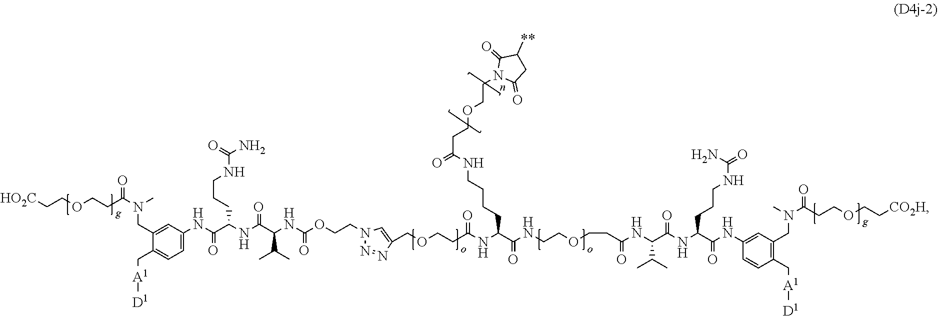
A¹ and A² are each independently selected from a bond, 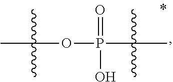 [SMILES: *OP(*)(=O)O]
and —O—C(═O)—*, wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 2 and 5);
n is an integer between 1 and 12 (e.g., between 2 and 5); and [symbol]
indicates the point of attachment to the Ab.

283. The compound or pharmaceutically acceptable salt thereof according to claim 258, of formula (D5a-1): 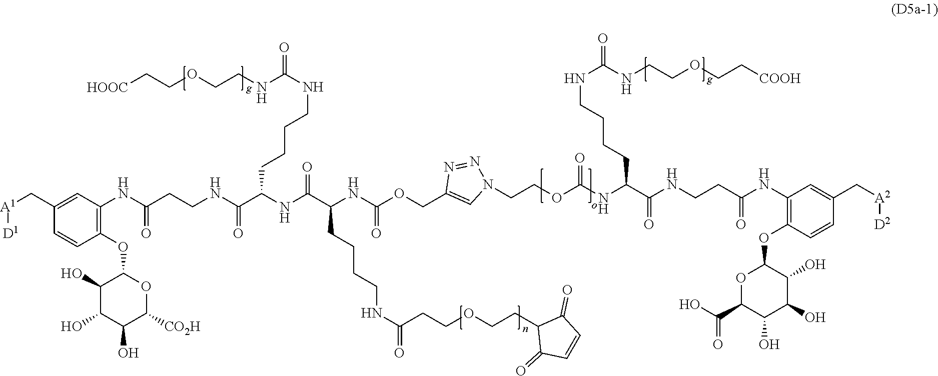
wherein:
A¹ and A² are each independently selected from a bond, 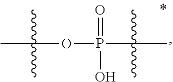 [SMILES: *OP(*)(=O)O]
and —O—C(═O)—*, wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 1 and 3); and
n is an integer between 1 and 12 (e.g., between 5 and 10).

284. The conjugate according to claim 279, of formula (D5a-2): 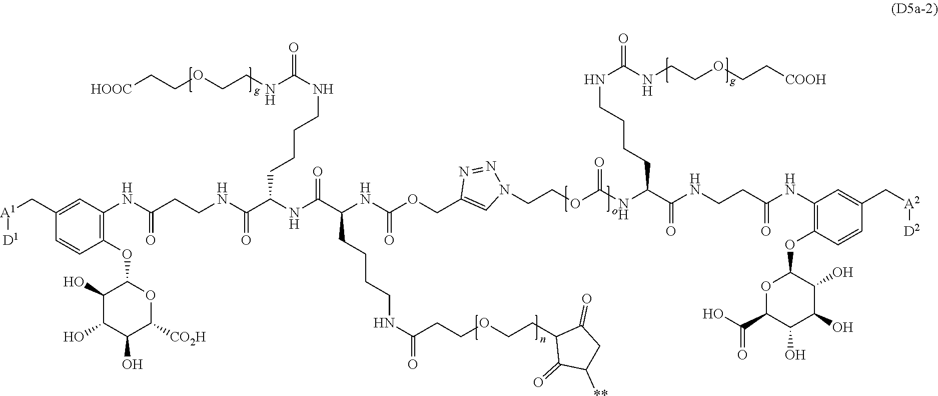
wherein:
A¹ and A² are each independently selected from a bond, 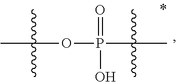 [SMILES: *OP(*)(=O)O]
and —O—C(═O)—*, wherein * in A¹ and A² indicates the point of attachment to D¹ or D²;
g for each occurrence is independently an integer between 20 and 30 (e.g., 20, 21, 22, 23, 24, 25, 26, 27, 28, 29 or 30);
o for each occurrence is independently an integer between 1 and 9 (e.g., between 1 and 3);
n is an integer between 1 and 12 (e.g., between 5 and 10); and [symbol]
indicates the point of attachment to the Ab;
or represented by the following formulae: 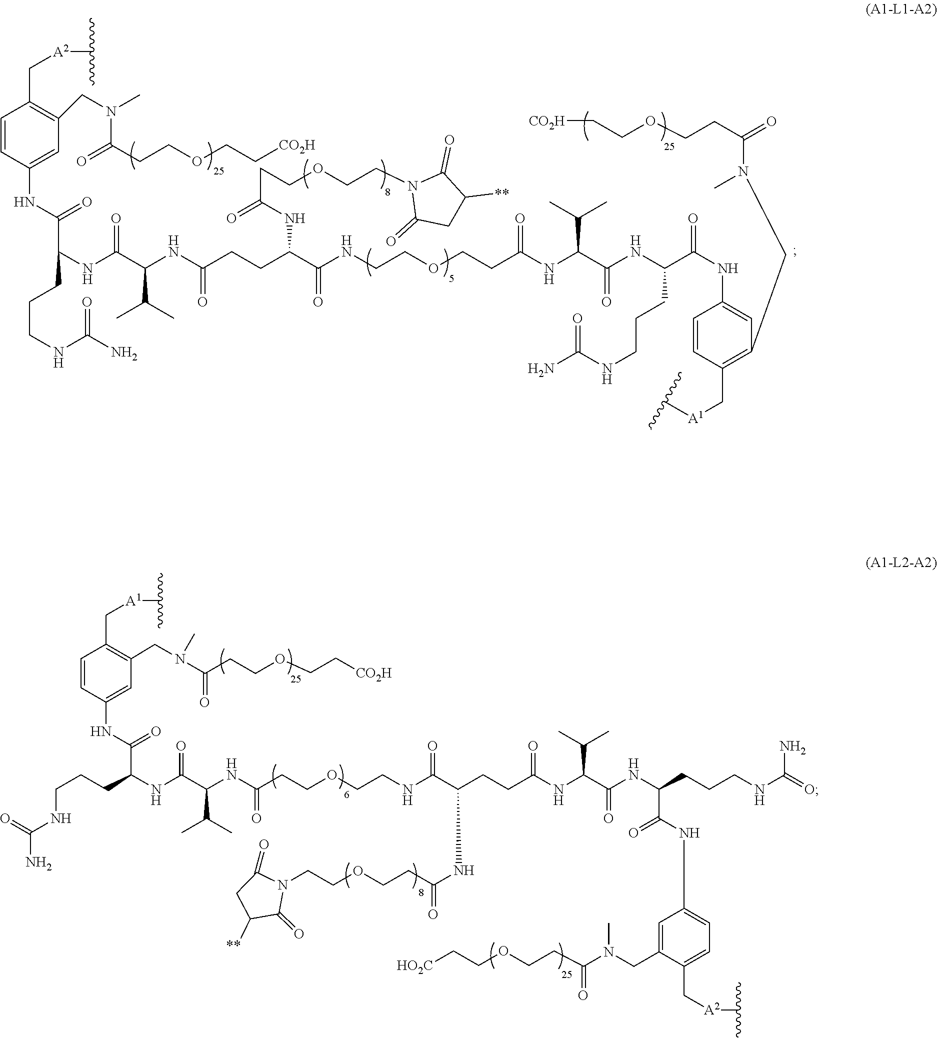 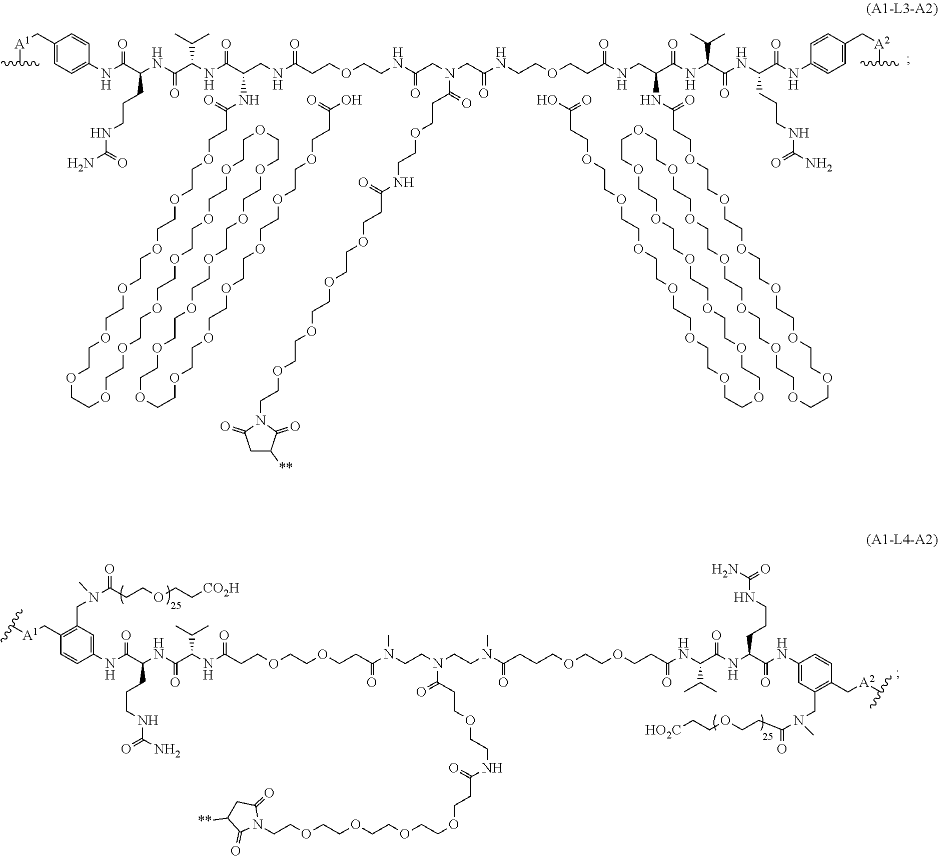 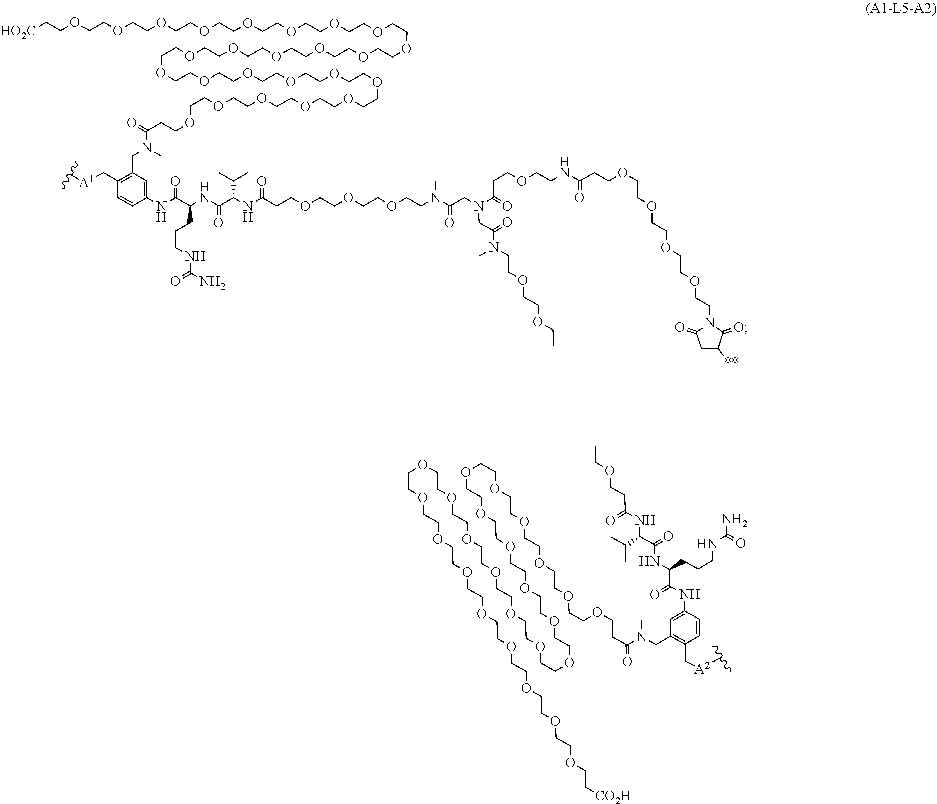 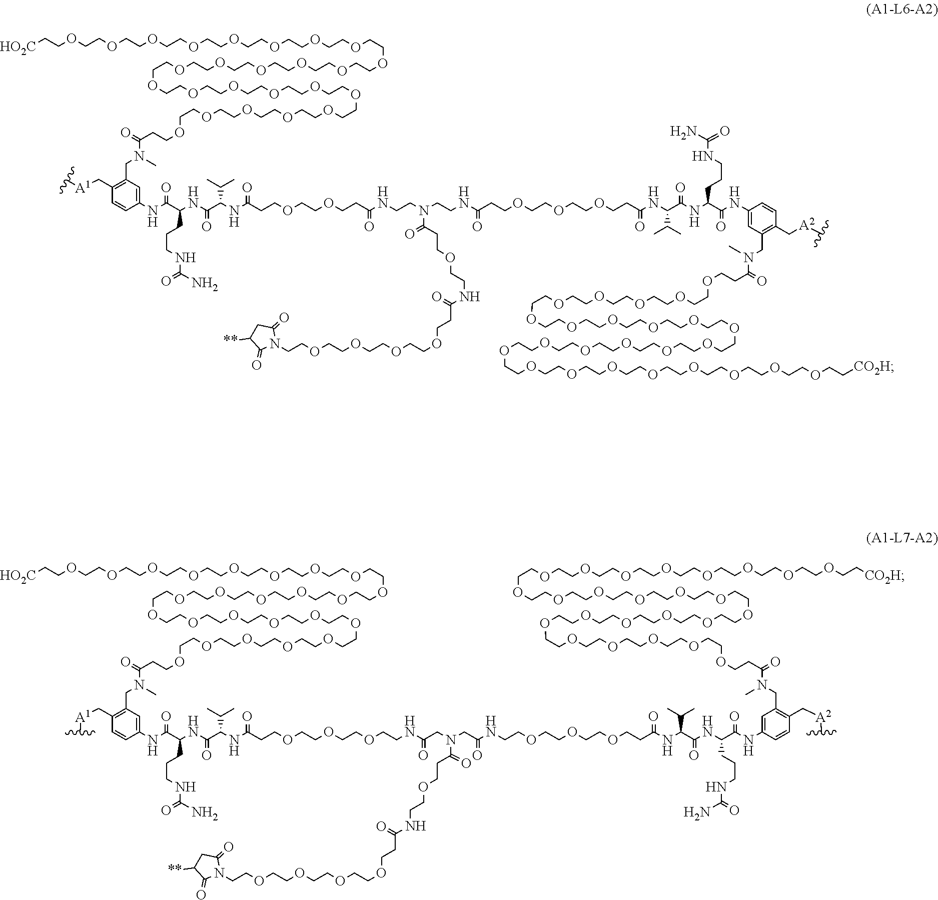 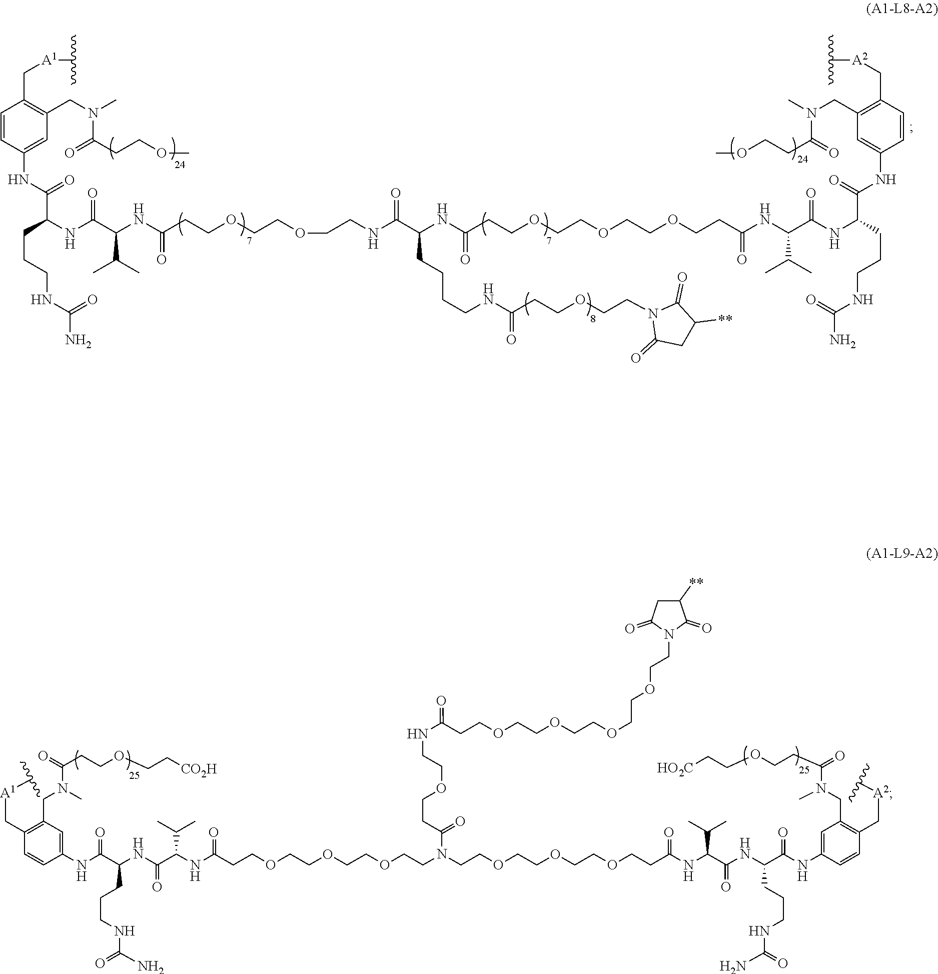 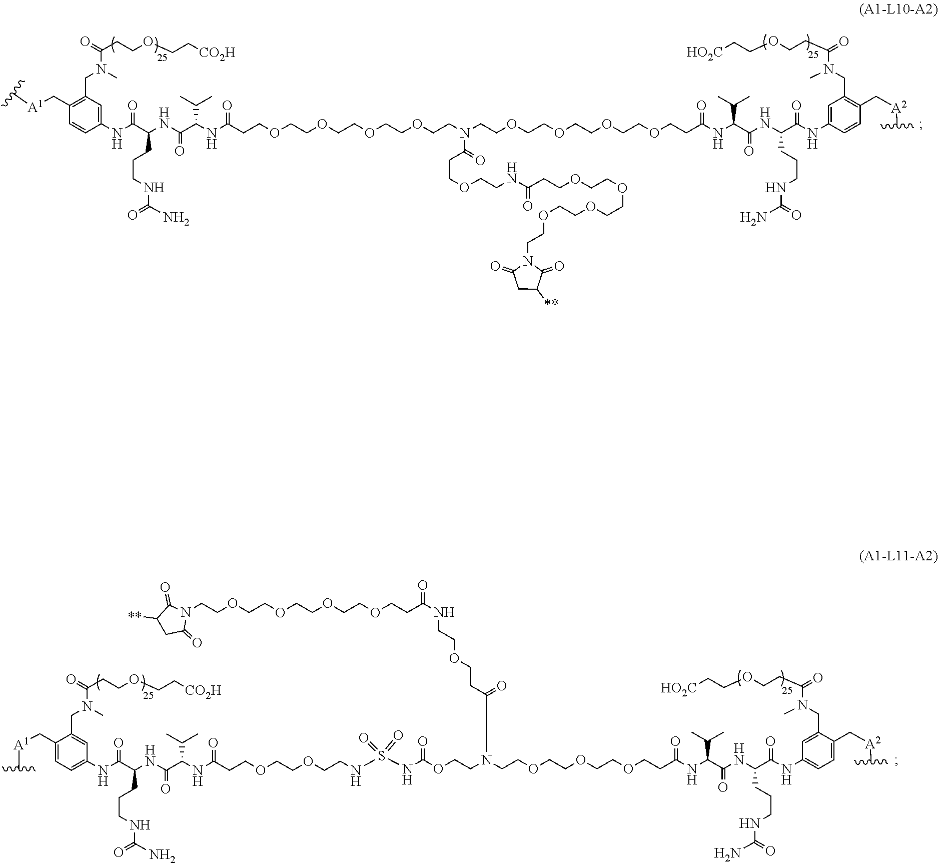 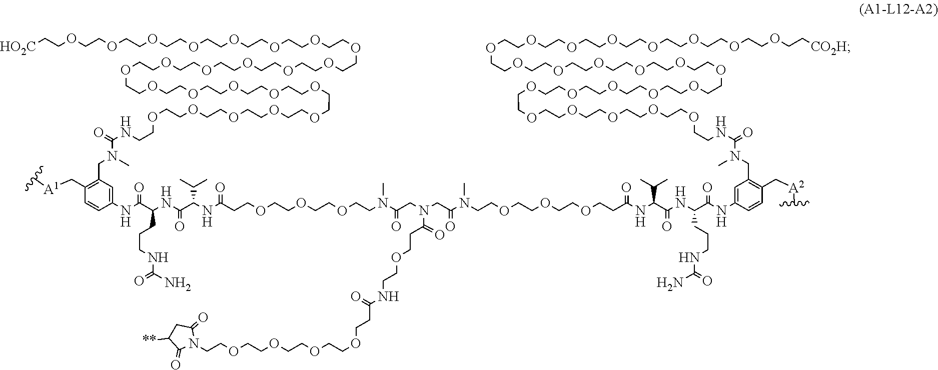 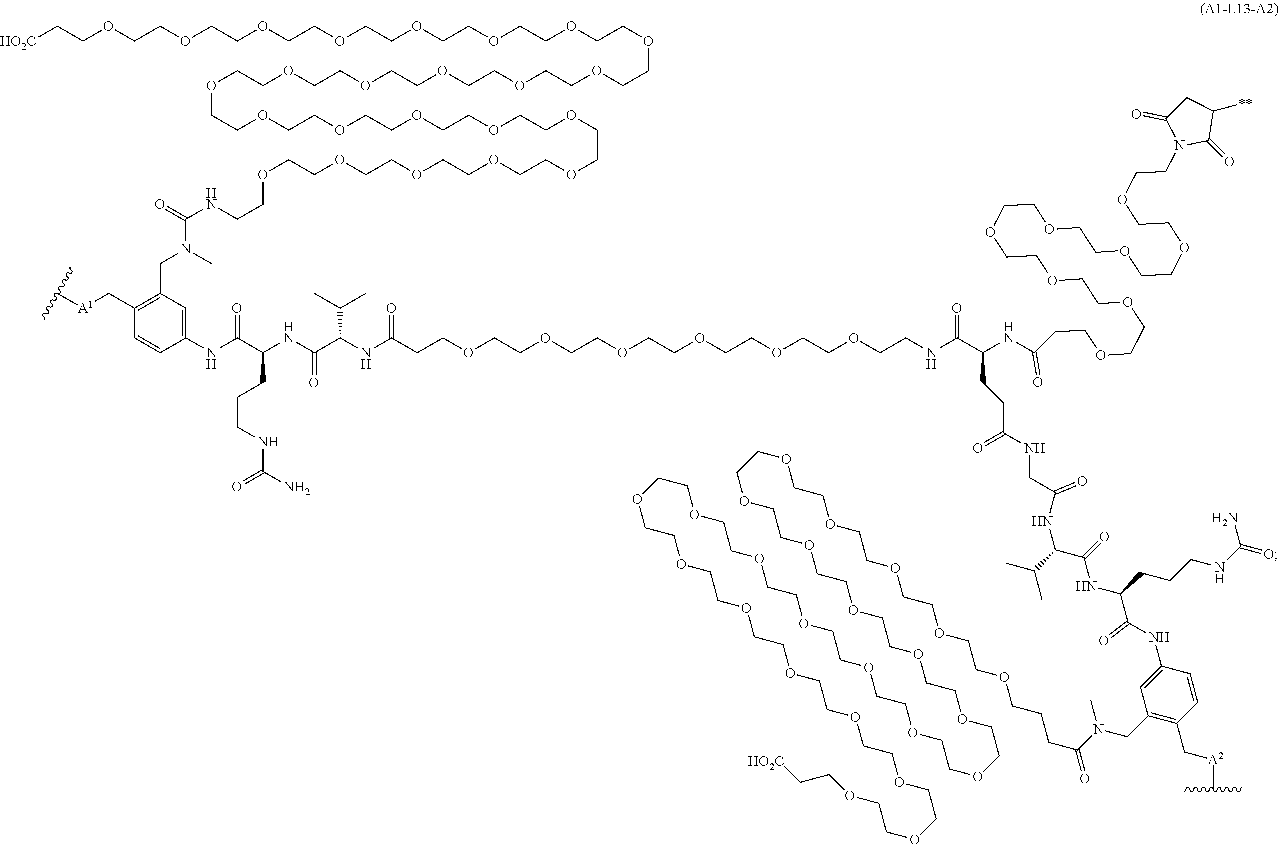 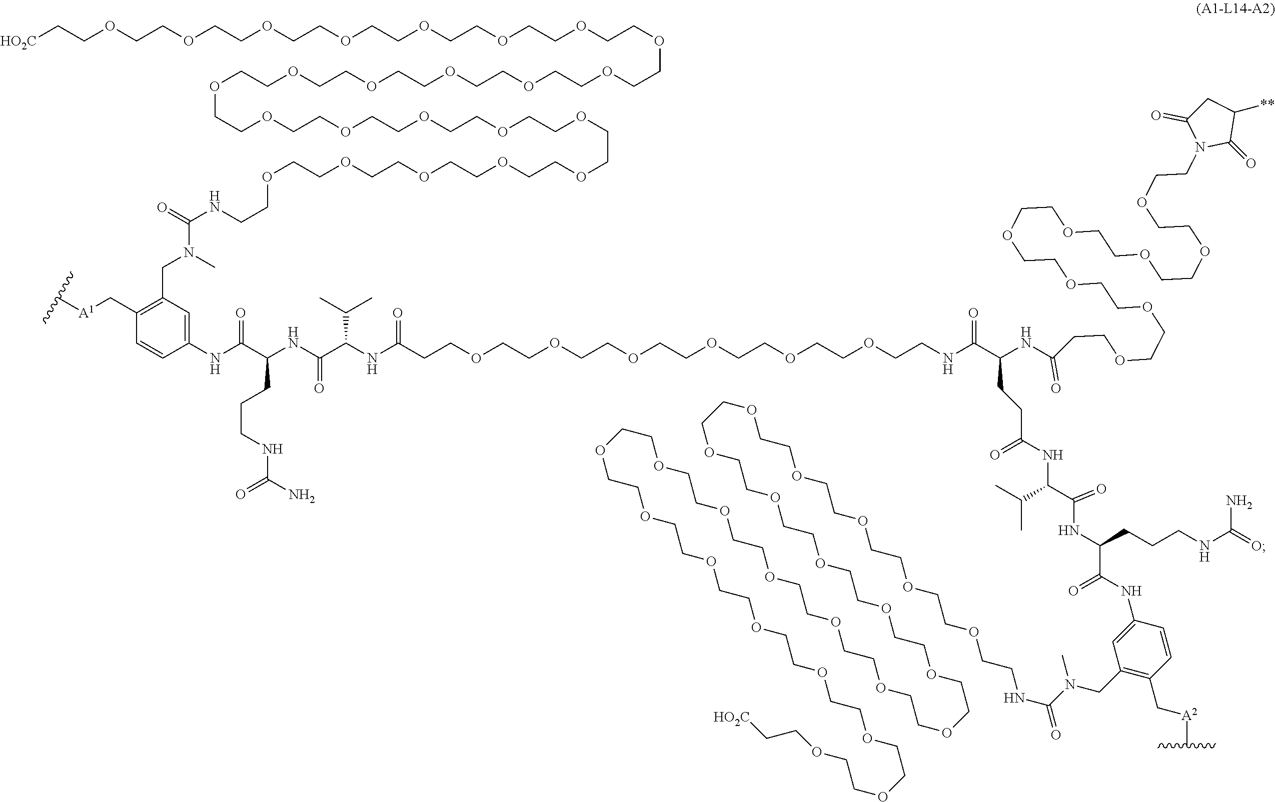 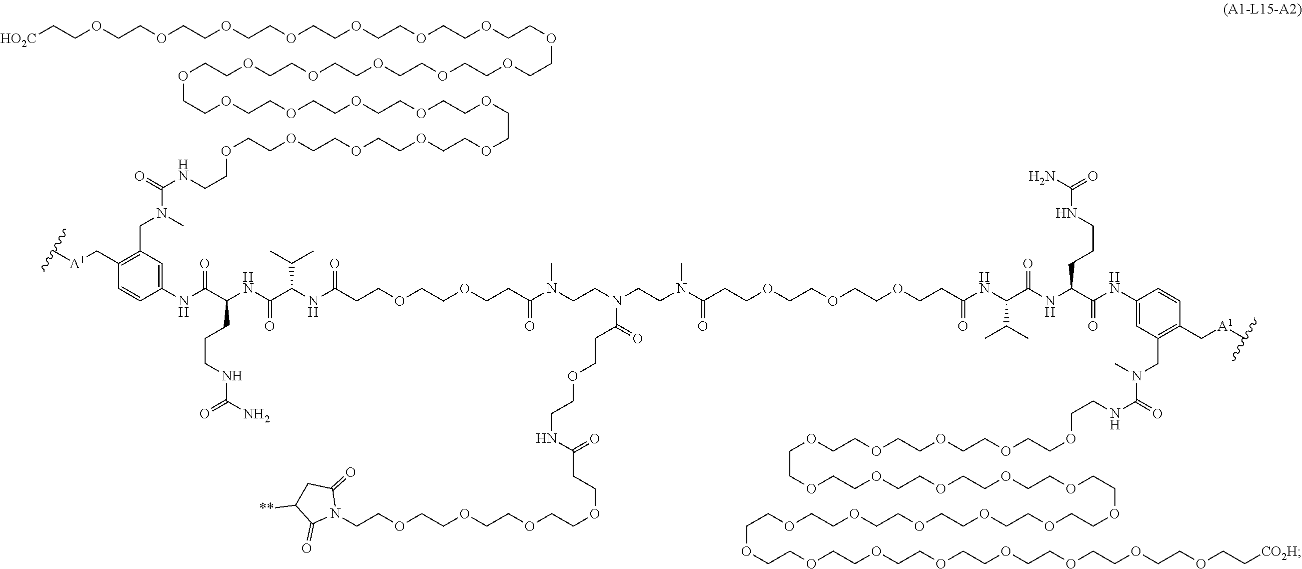 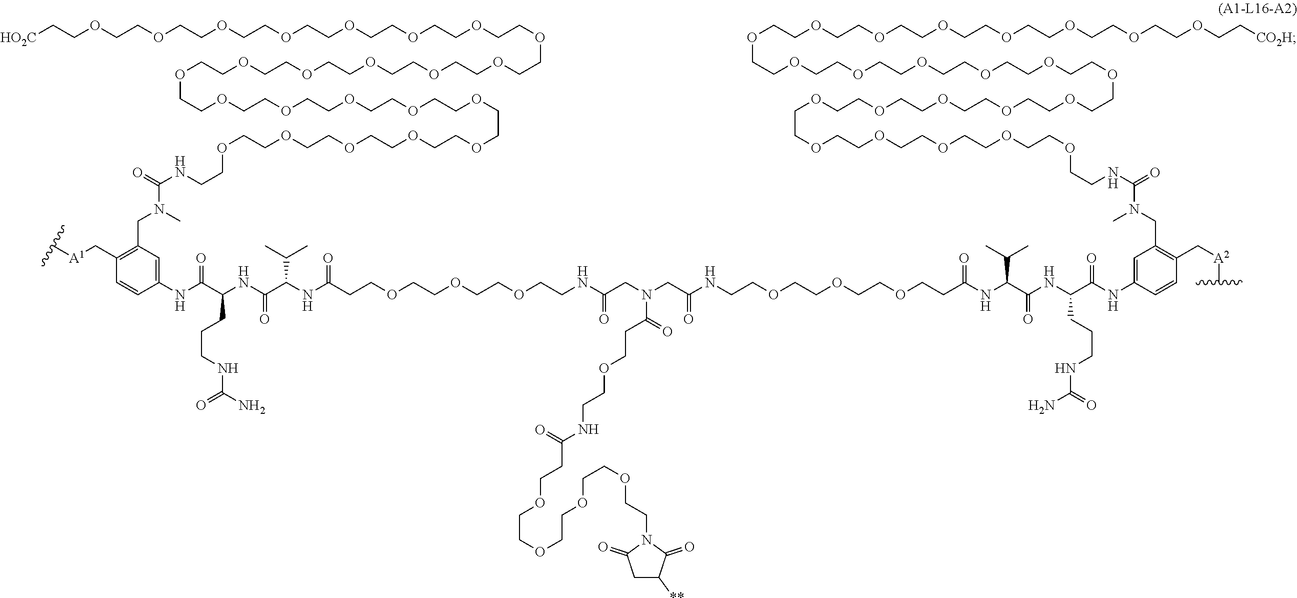 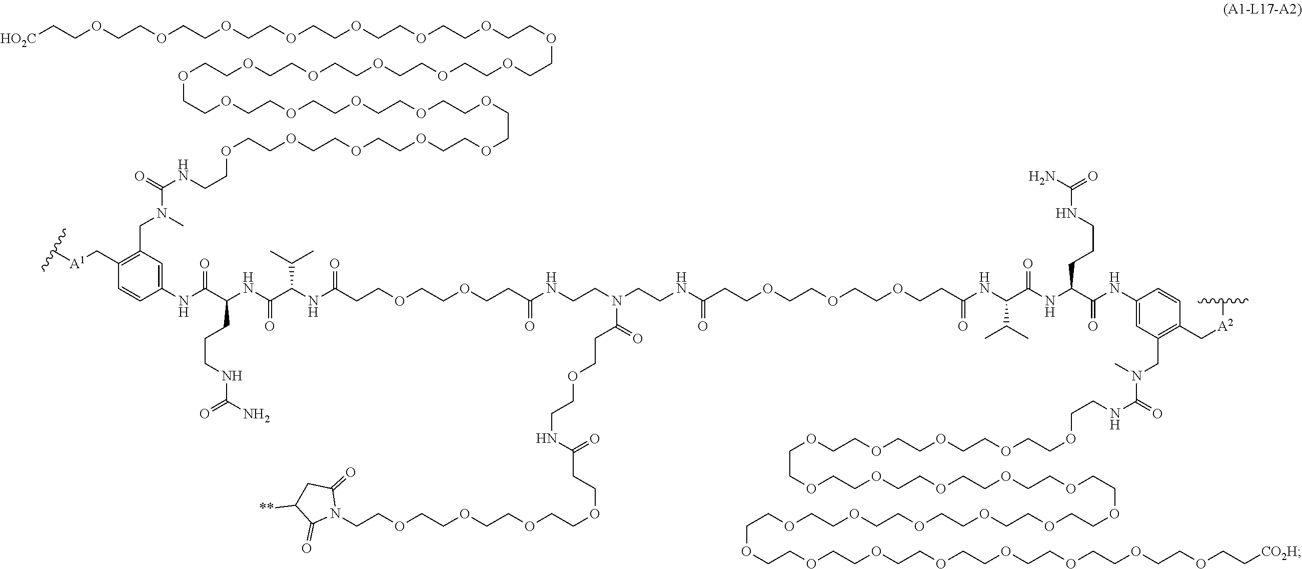 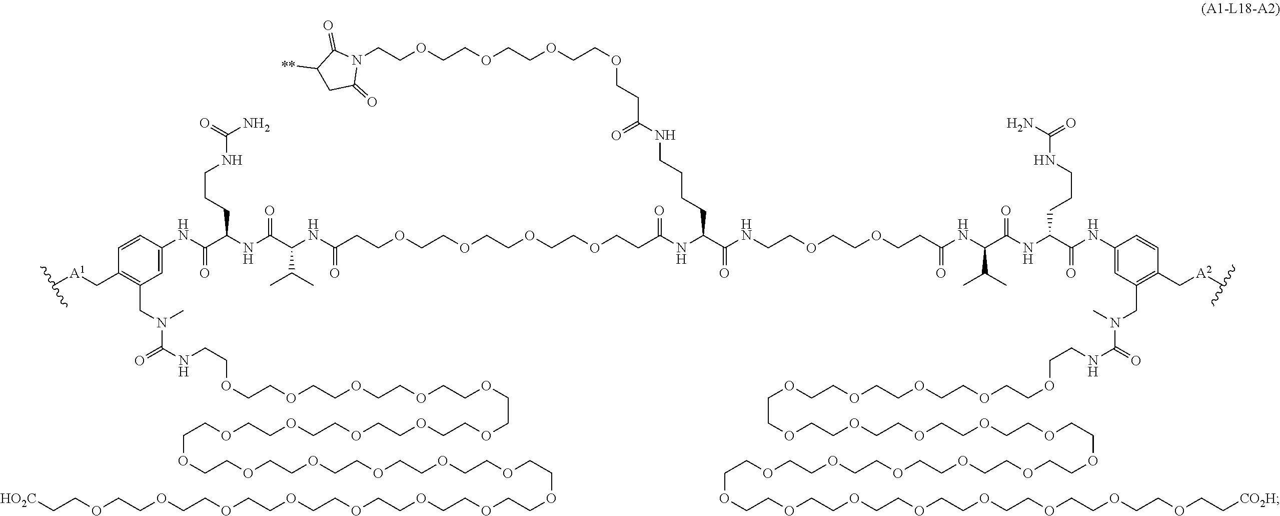 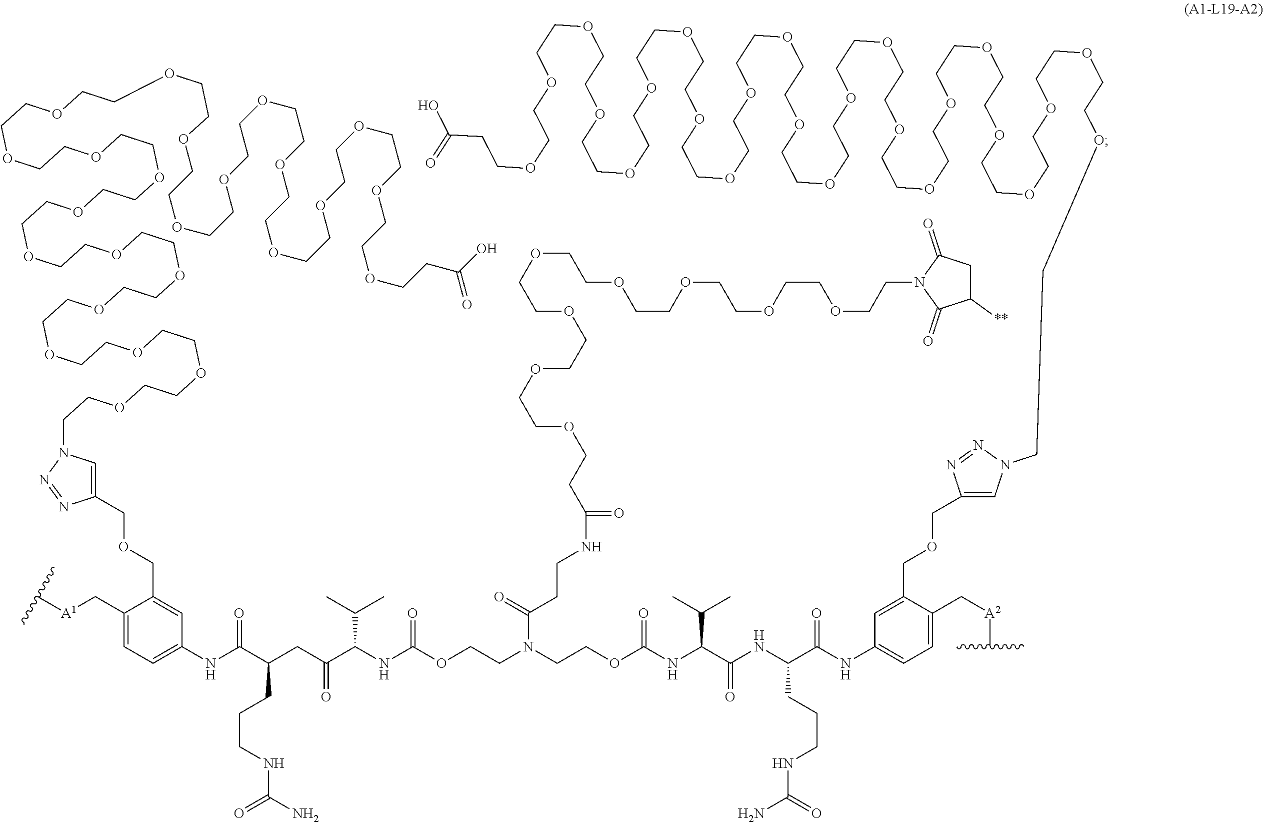 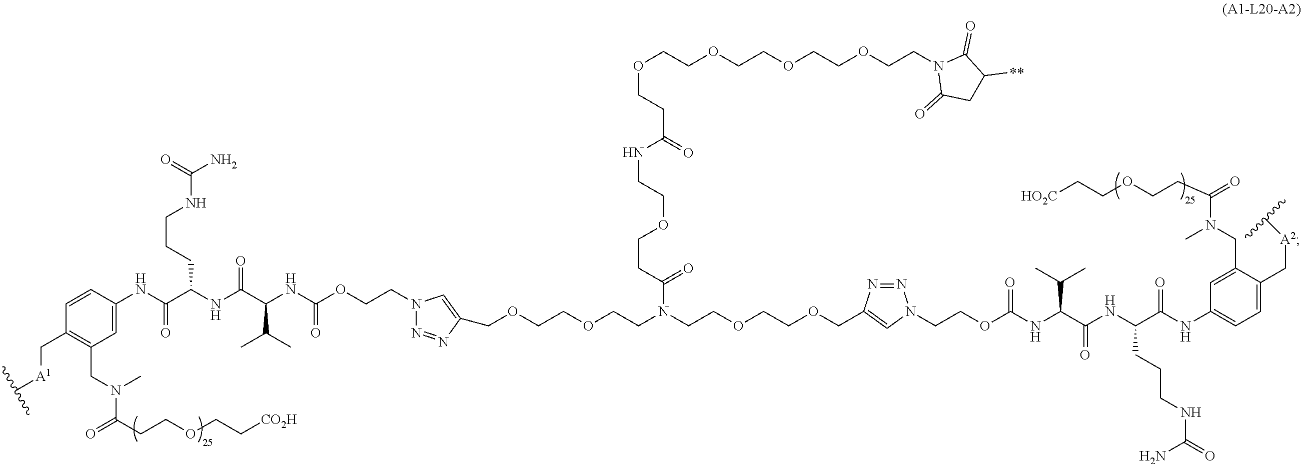 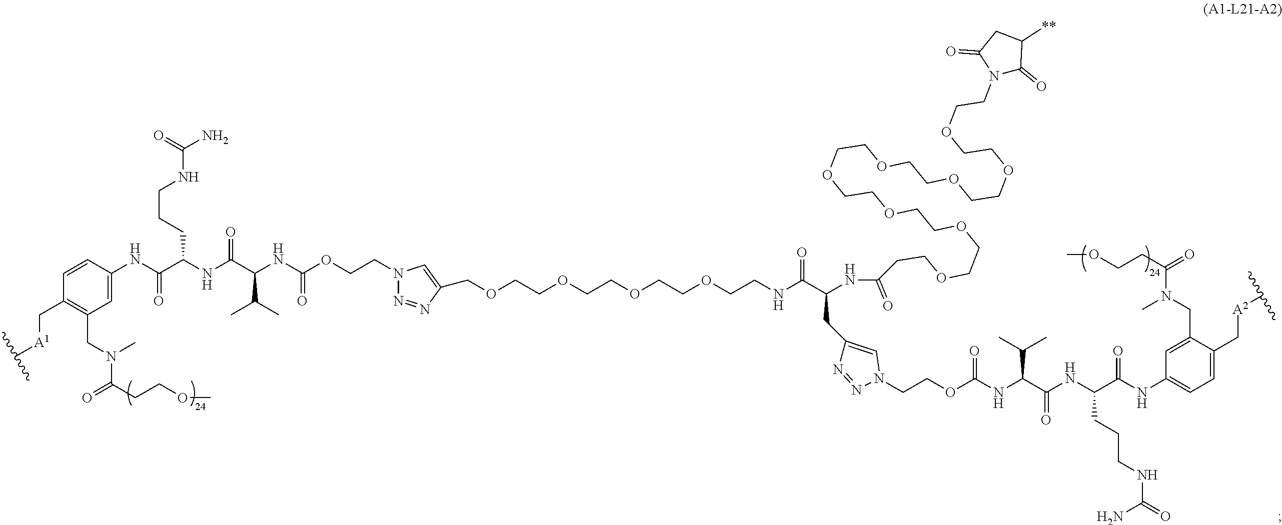 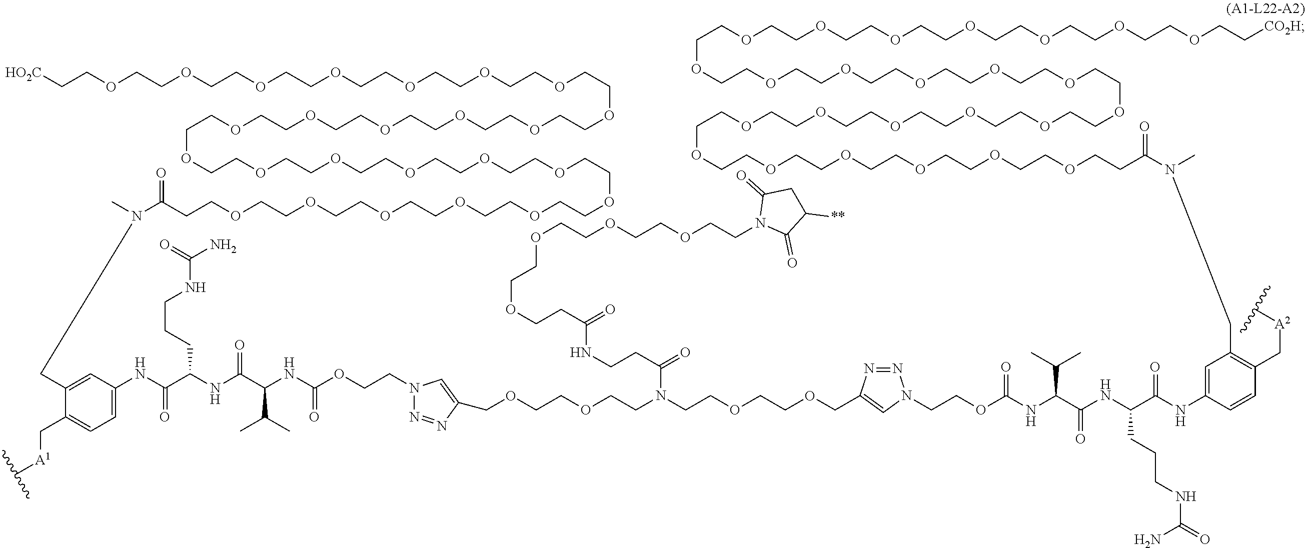 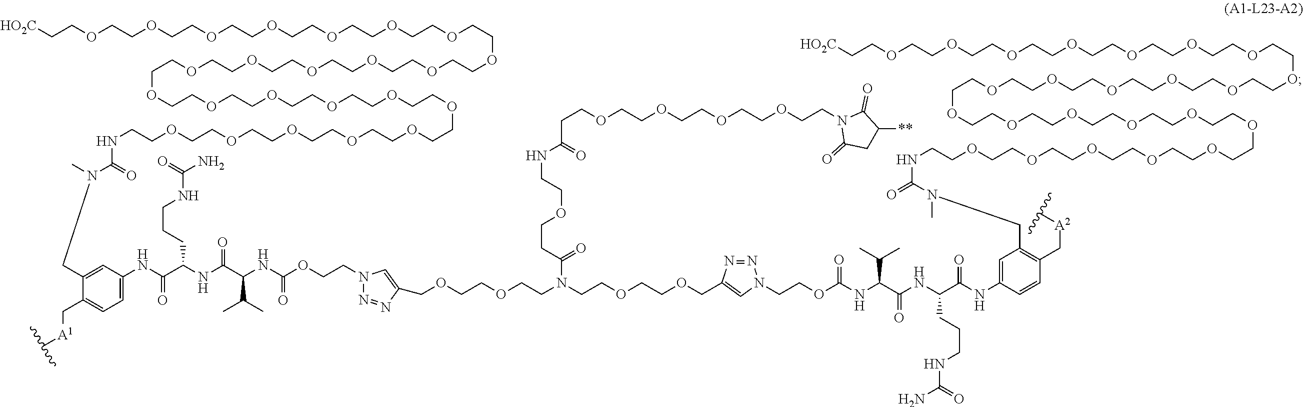 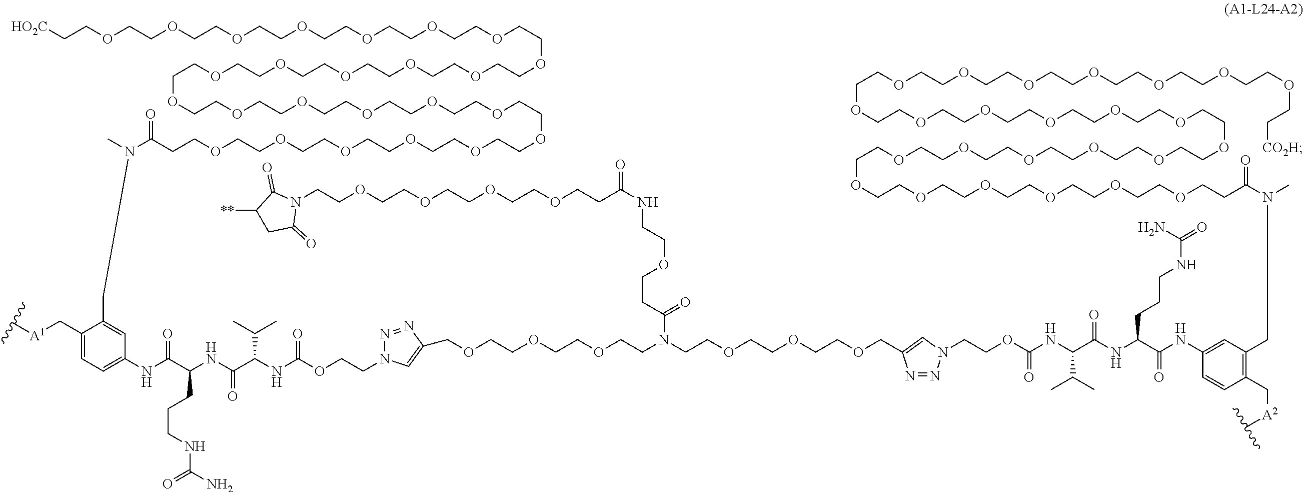 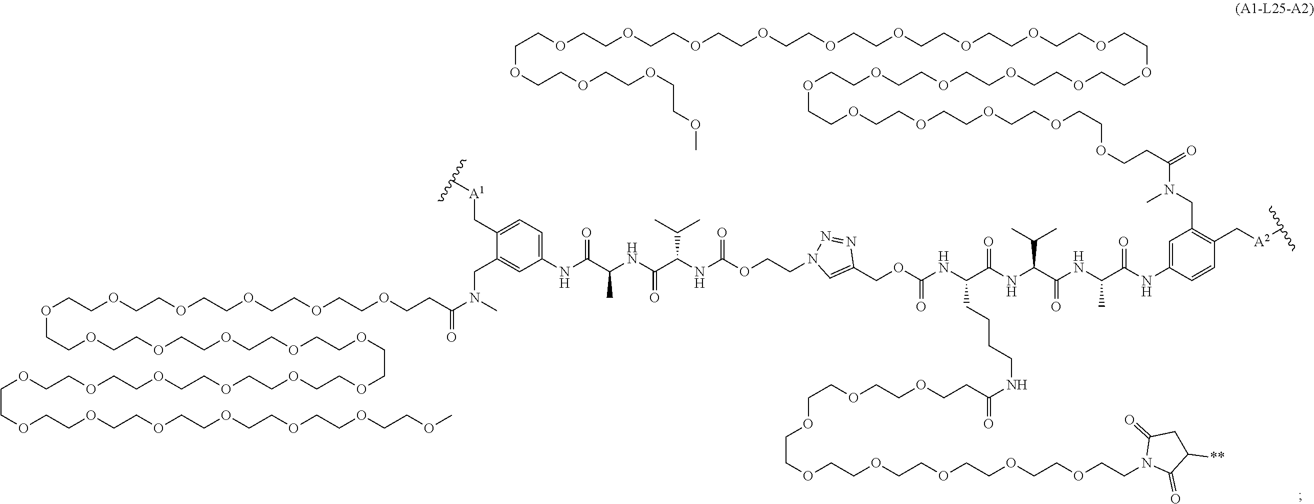 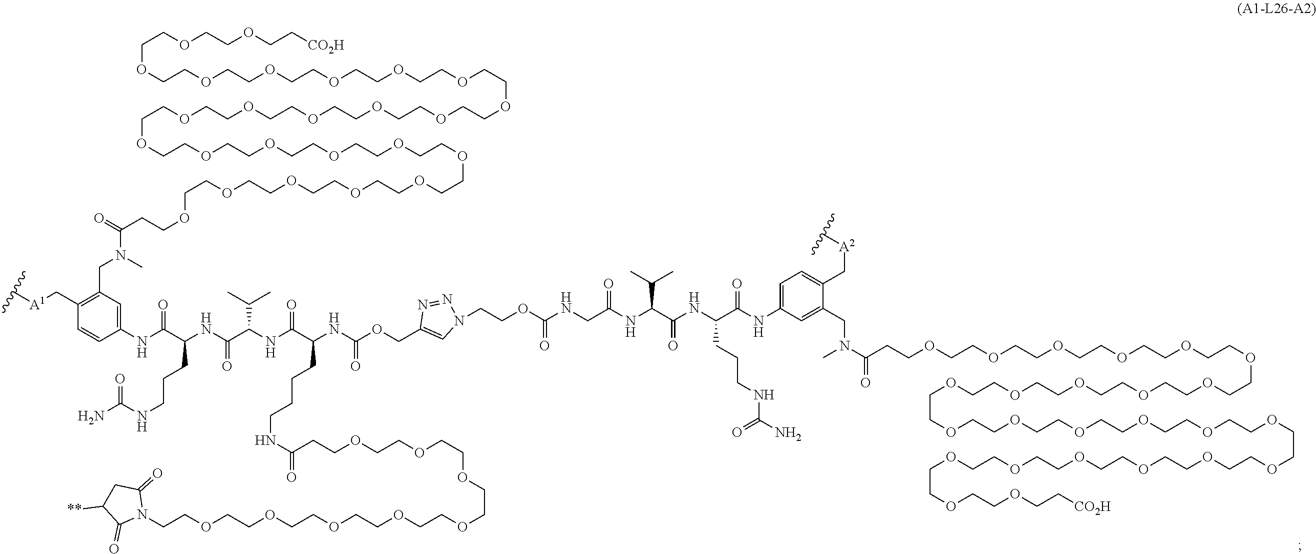 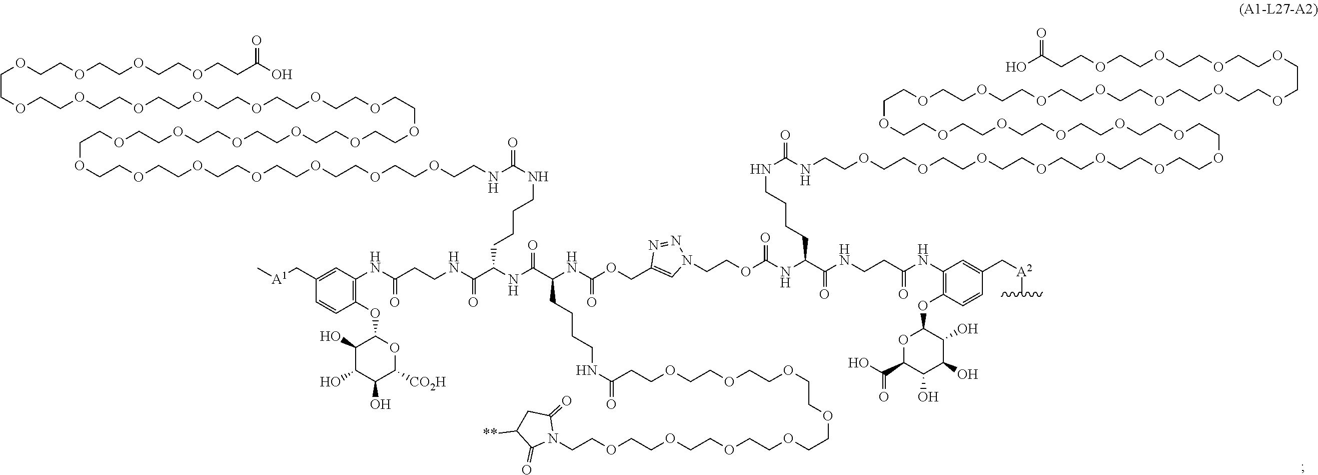 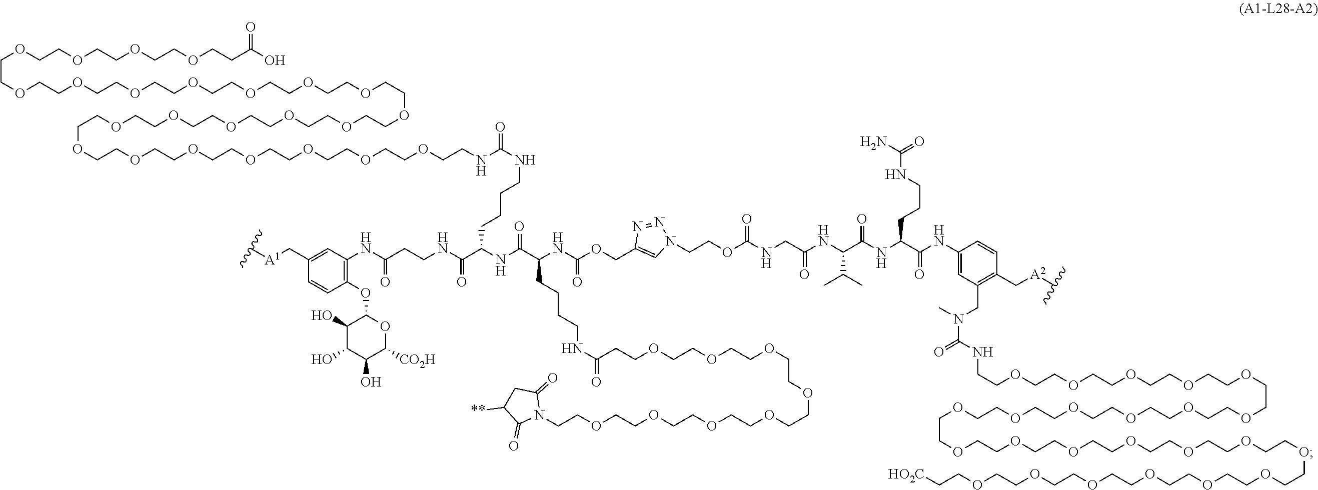 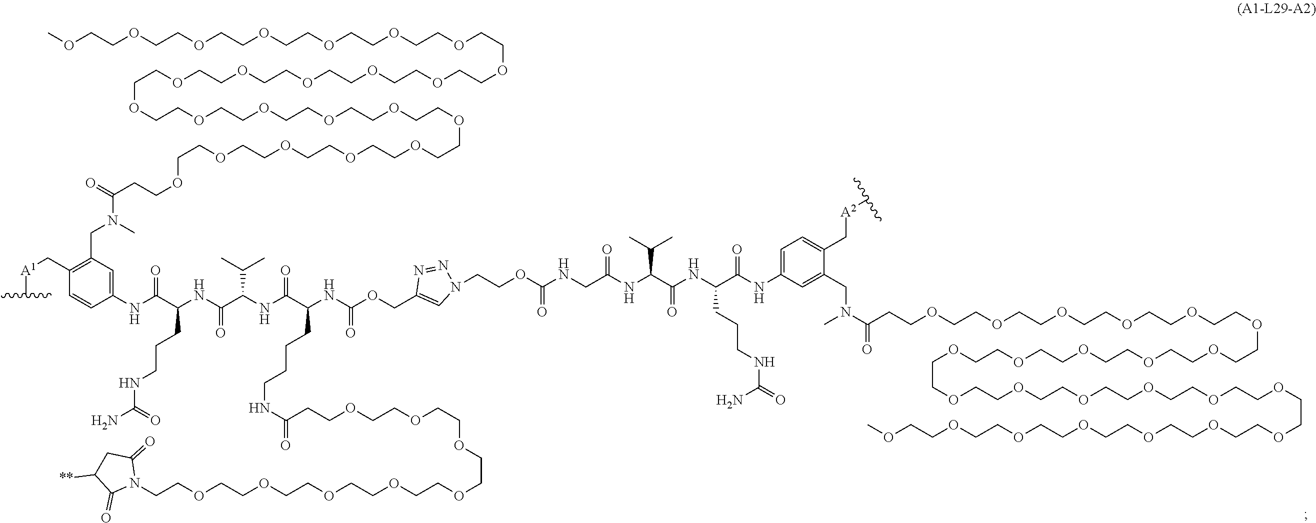 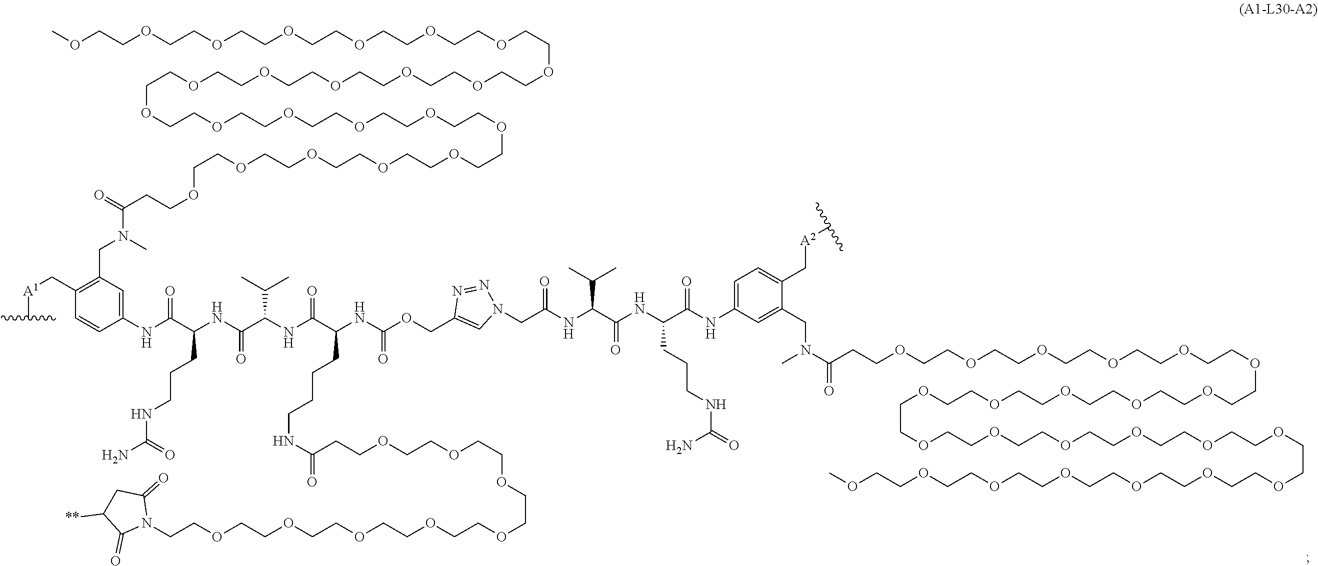 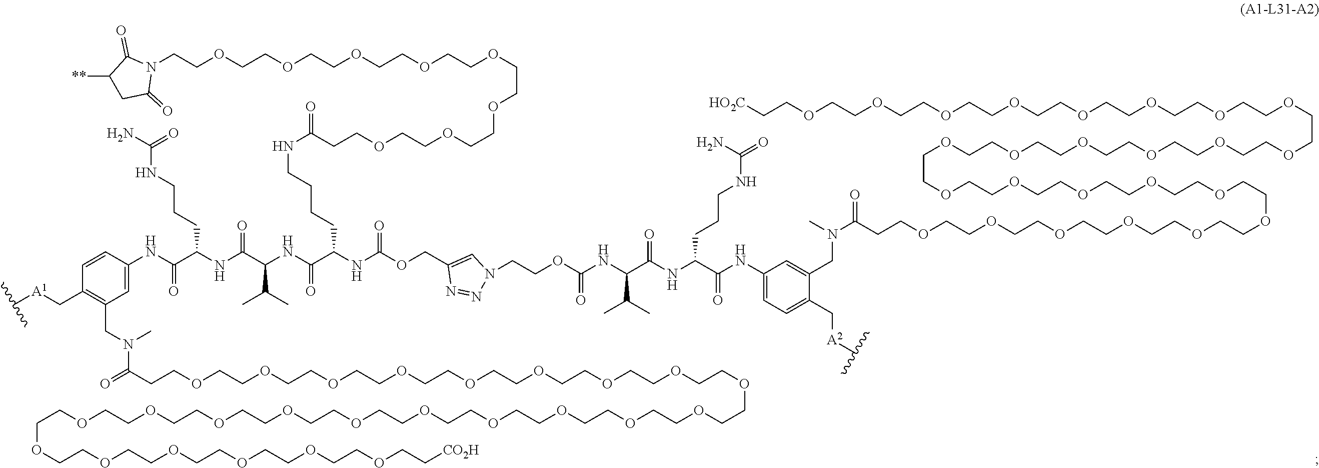 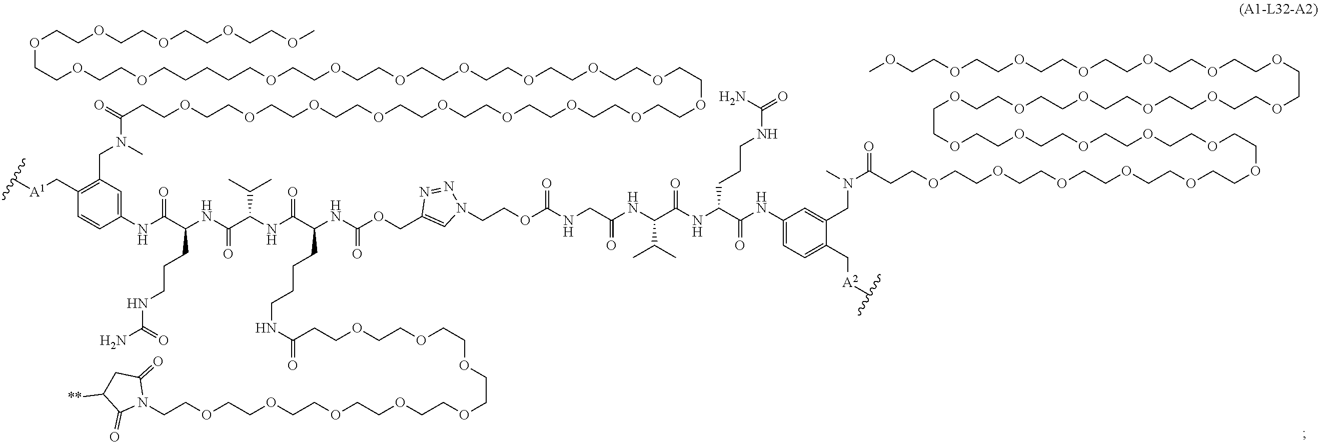 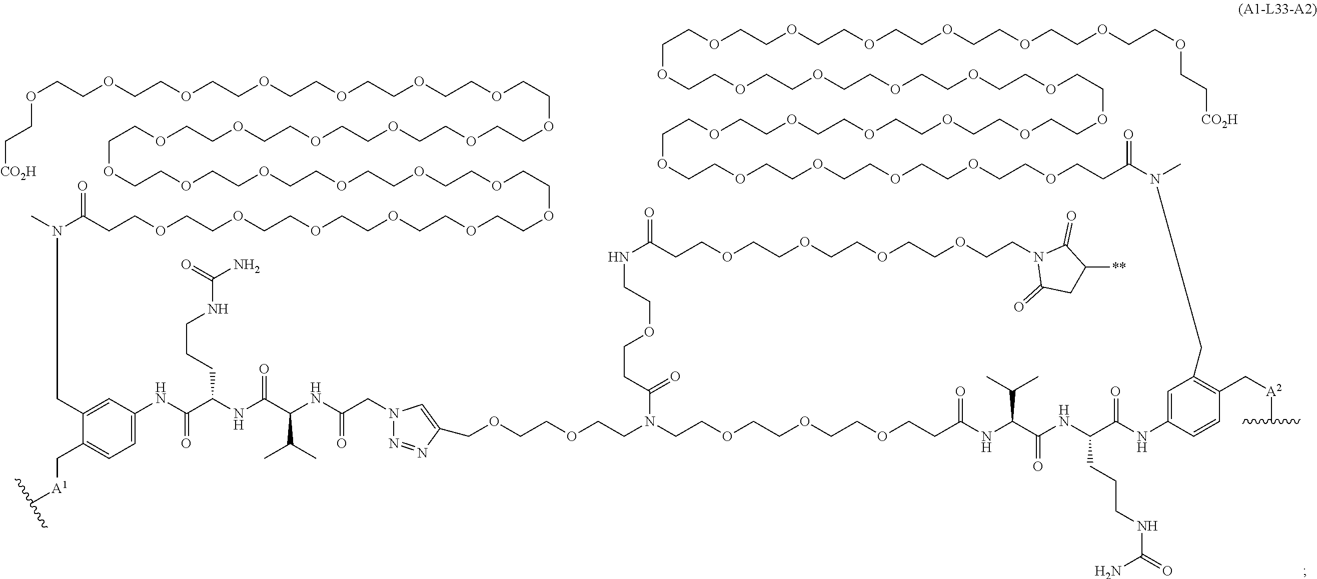 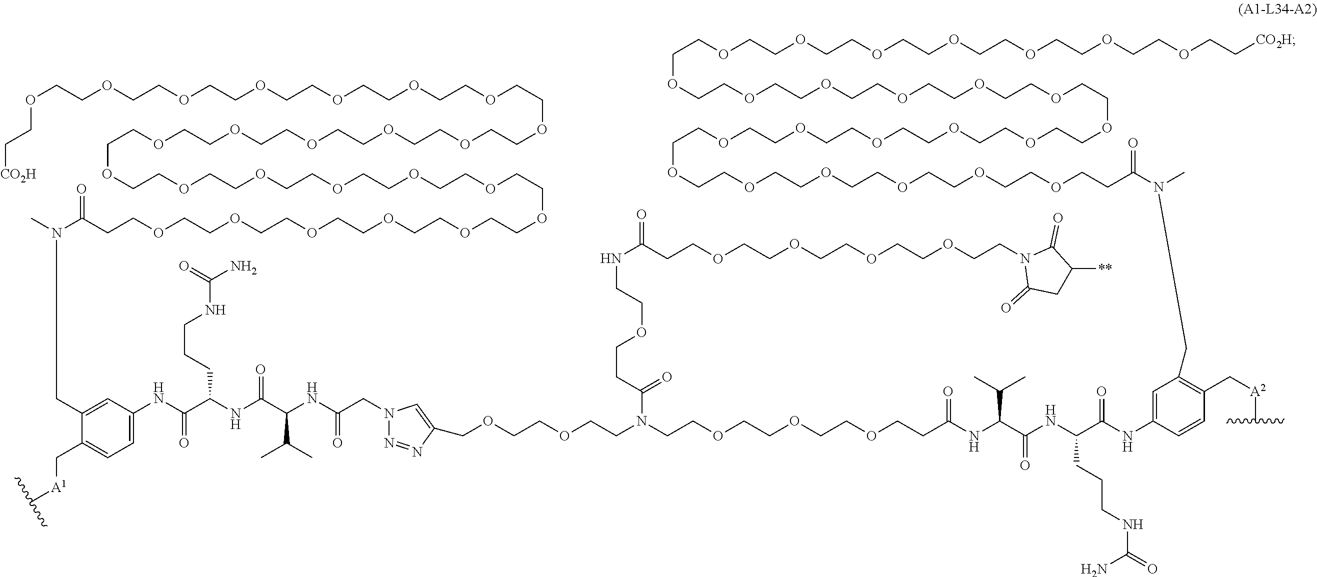 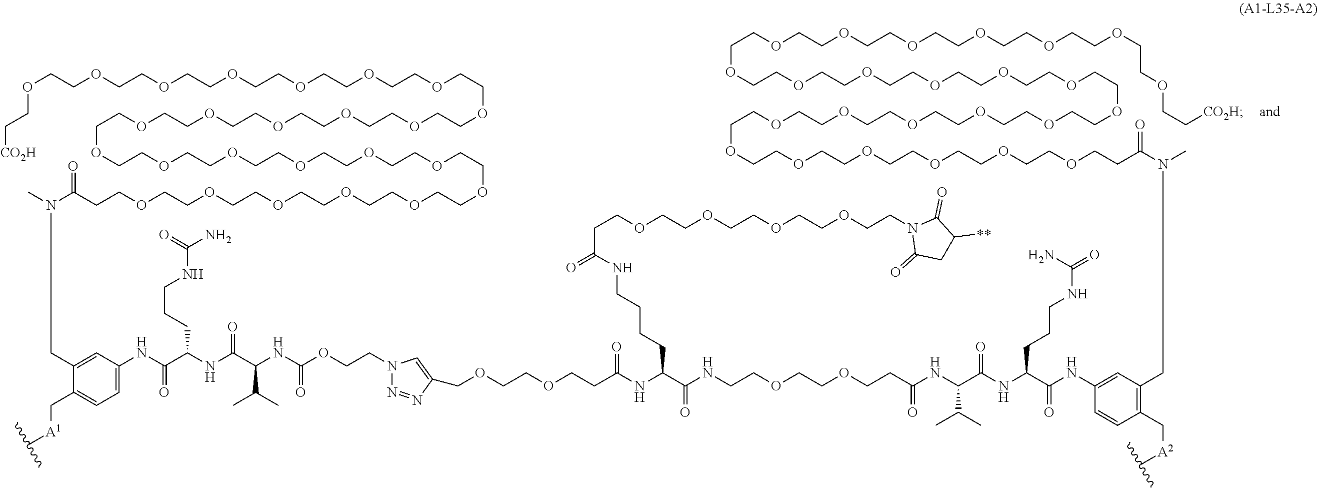 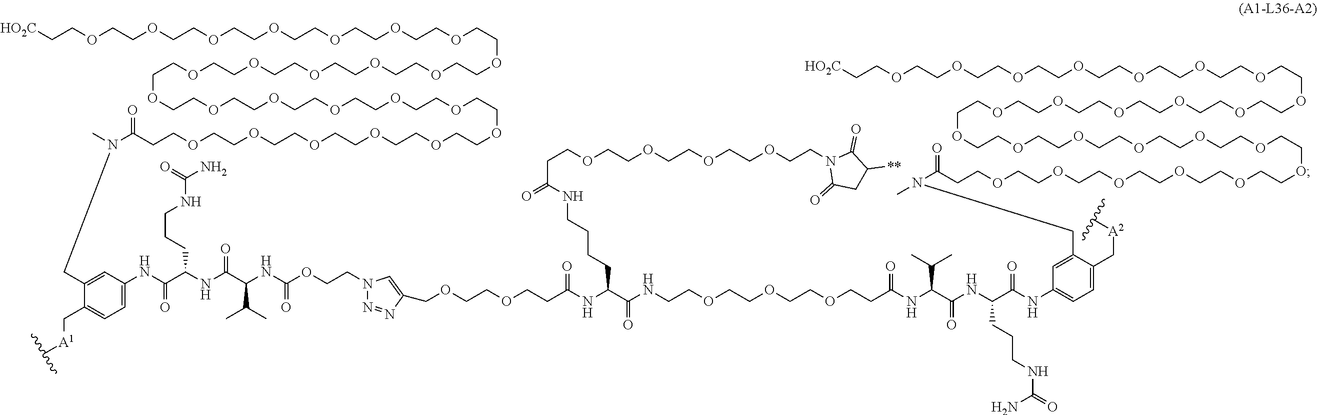
wherein A¹ or A² are each independently selected from a bond, 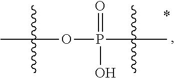 [SMILES: *OP(*)(=O)O]
and —OC(═O)—*, wherein * indicates the point of attachment to D¹ or D²; [symbol] indicates the point of attachment to the Ab; and [symbol] indicates the point of direct attachment to D¹ or D².

285. A pharmaceutical composition comprising the conjugate according to claim 279, and a pharmaceutically acceptable carrier.

286. A pharmaceutical composition comprising the conjugate according to claim 284, and a pharmaceutically acceptable carrier.


SEQUENCE LISTING (402 sequences)

SEQ ID NO: 1 - protein, 121 aa, synthetic construct
  REGION 1..121: note = Synthetic
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 2 - protein, 107 aa, synthetic construct
  REGION 1..107: note = Synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 3 - protein, 118 aa, synthetic construct
  REGION 1..118: note = Synthetic
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 4 - protein, 112 aa, synthetic construct
  REGION 1..112: note = Synthetic
NIMLTQSPSSLAVSAGEKVTMSCKSSQSVFFSSSQKNYLAWYQQIPGQSPKLLIYWASTRESGVPDRFTGSGSGTDFTLIISSVQSEDLAIYYCHQYLSSRTFGGGTKLEIK

SEQ ID NO: 5 - protein, 116 aa, synthetic construct [sequence omitted]

SEQ ID NO: 6 - protein, 111 aa, synthetic construct [sequence omitted]

SEQ ID NO: 7 - protein, 123 aa, synthetic construct
  REGION 1..123: note = Synthetic
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSS

SEQ ID NO: 8 - protein, 106 aa, synthetic construct
  REGION 1..106: note = Synthetic
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIK

SEQ ID NO: 9 - protein, 120 aa, synthetic construct [sequence omitted]

SEQ ID NO: 10 - protein, 109 aa, synthetic construct
  REGION 1..109: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 11 - protein, 122 aa, synthetic construct [sequence omitted]

SEQ ID NO: 12 - protein, 106 aa, synthetic construct
  REGION 1..106: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEI

SEQ ID NO: 13 - protein, 119 aa, synthetic construct
  REGION 1..119: note = Synthetic
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSS

SEQ ID NO: 14 - protein, 107 aa, synthetic construct
  REGION 1..107: note = Synthetic
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIK

SEQ ID NO: 15 - protein, 10 aa, synthetic construct
  REGION 1..10: note = Synthetic
GGTFSNYWMH

SEQ ID NO: 16 - protein, 17 aa, synthetic construct
  REGION 1..17: note = Synthetic
ATYRGHSDTYYNQKFKG

SEQ ID NO: 17 - protein, 12 aa, synthetic construct
  REGION 1..12: note = Synthetic
GAIYNGYDVLDN

SEQ ID NO: 18 - protein, 11 aa, synthetic construct
  REGION 1..11: note = Synthetic
SASQDISNYLN

SEQ ID NO: 19 - protein, 7 aa, synthetic construct
  REGION 1..7: note = Synthetic
YTSNLHS

SEQ ID NO: 20 - protein, 9 aa, synthetic construct
  REGION 1..9: note = Synthetic
QQYRKLPWT

SEQ ID NO: 21 - protein, 10 aa, synthetic construct
  REGION 1..10: note = Synthetic
GYTFTSYYIH

SEQ ID NO: 22 - protein, 17 aa, synthetic construct
  REGION 1..17: note = Synthetic
VIYPGNDDISYNQKFKG

SEQ ID NO: 23 - protein, 9 aa, synthetic construct
  REGION 1..9: note = Synthetic
EVRLRYFDV

SEQ ID NO: 24 - protein, 17 aa, synthetic construct
  REGION 1..17: note = Synthetic
KSSQSVFFSSSQKNYLA

SEQ ID NO: 25 - protein, 7 aa, synthetic construct
  REGION 1..7: note = Synthetic
WASTRES

SEQ ID NO: 26 - protein, 8 aa, synthetic construct
  REGION 1..8: note = Synthetic
HQYLSSRT

SEQ ID NO: 27 - protein, 10 aa, synthetic construct
  REGION 1..10: note = Synthetic
GYTITDSNIH

SEQ ID NO: 28 - protein, 17 aa, synthetic construct
  REGION 1..17: note = Synthetic
YIYPYNGGTDYNQKFKN

SEQ ID NO: 29 - protein, 7 aa, synthetic construct
  REGION 1..7: note = Synthetic
GNPWLAY

SEQ ID NO: 30 - protein, 15 aa, synthetic construct
  REGION 1..15: note = Synthetic
RASESLDNYGIRFLT

SEQ ID NO: 31 - protein, 7 aa, synthetic construct
  REGION 1..7: note = Synthetic
AASNQGS

SEQ ID NO: 32 - protein, 9 aa, synthetic construct
  REGION 1..9: note = Synthetic
QQTKEVPWS

SEQ ID NO: 33 - protein, 17 aa, synthetic construct
  REGION 1..17: note = Synthetic
TCAISGDSVSSQSAAWN

SEQ ID NO: 34 - protein, 18 aa, synthetic construct
  REGION 1..18: note = Synthetic
RIYYRSKWYNDYALSVKS

SEQ ID NO: 35 - protein, 11 aa, synthetic construct
  REGION 1..11: note = Synthetic
GEGYGREGFAI

SEQ ID NO: 36 - protein, 11 aa, synthetic construct
  REGION 1..11: note = Synthetic
RASQTISNTLA

SEQ ID NO: 37 - protein, 7 aa, synthetic construct
  REGION 1..7: note = Synthetic
AASNLQS

SEQ ID NO: 38 - protein, 8 aa, synthetic construct
  REGION 1..8: note = Synthetic
QQYLSWFT

SEQ ID NO: 39 - protein, 10 aa, synthetic construct
  REGION 1..10: note = Synthetic
GFNIKDTYIH

SEQ ID NO: 40 - protein, 17 aa, synthetic construct
  REGION 1..17: note = Synthetic
RIYPTNGYTRYADSVKG

SEQ ID NO: 41 - protein, 11 aa, synthetic construct
  REGION 1..11: note = Synthetic
WGGDGFYAMDV

SEQ ID NO: 42 - protein, 12 aa, synthetic construct
  REGION 1..12: note = Synthetic
RASQDVNTAVAW

SEQ ID NO: 43 - protein, 7 aa, synthetic construct
  REGION 1..7: note = Synthetic
SASFLES

SEQ ID NO: 44 - protein, 9 aa, synthetic construct
  REGION 1..9: note = Synthetic
QQHYTTPPT

SEQ ID NO: 45 - protein, 10 aa, synthetic construct
  REGION 1..10: note = Synthetic
GFTFNSFAMS

SEQ ID NO: 46 - protein, 17 aa, synthetic construct
  REGION 1..17: note = Synthetic
AISGSGGGTYYADSVKG

SEQ ID NO: 47 - protein, 13 aa, synthetic construct
  REGION 1..13: note = Synthetic
DKILWFGEPVFDY

SEQ ID NO: 48 - protein, 11 aa, synthetic construct
  REGION 1..11: note = Synthetic
RASQSVSSYLA

SEQ ID NO: 49 - protein, 7 aa, synthetic construct
  REGION 1..7: note = Synthetic
DASNRAT

SEQ ID NO: 50 - protein, 9 aa, synthetic construct
  REGION 1..9: note = Synthetic
QQRSNWPPT

SEQ ID NO: 51 - protein, 10 aa, synthetic construct
  REGION 1..10: note = Synthetic
GYTFTDFGMN

SEQ ID NO: 52 - protein, 17 aa, synthetic construct
  REGION 1..17: note = Synthetic
WINTFTGEPSYGNVFKG

SEQ ID NO: 53 - protein, 10 aa, synthetic construct
  REGION 1..10: note = Synthetic
RHGNGNVFDS

SEQ ID NO: 54 - protein, 11 aa, synthetic construct
  REGION 1..11: note = Synthetic
RASQSIGSNIH

SEQ ID NO: 55 - protein, 7 aa, synthetic construct
  REGION 1..7: note = Synthetic
YTSESIS

SEQ ID NO: 56 - protein, 9 aa, synthetic construct
  REGION 1..9: note = Synthetic
QQSNSWPLT

SEQ ID NO: 57 - protein, 451 aa, synthetic construct
  REGION 1..451: note = Synthetic
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
  REGION 1..214: note = Synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 448 aa, synthetic construct
  REGION 1..448: note = Synthetic
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 60 - protein, 219 aa, synthetic construct
  REGION 1..219: note = Synthetic
NIMLTQSPSSLAVSAGEKVTMSCKSSQSVFFSSSQKNYLAWYQQIPGQSPKLLIYWASTRESGVPDRFTGSGSGTDFTLIISSVQSEDLAIYYCHQYLSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 446 aa, synthetic construct
  REGION 1..446: note = Synthetic
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 62 - protein, 218 aa, synthetic construct
  REGION 1..218: note = Synthetic
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 453 aa, synthetic construct
  REGION 1..453: note = Synthetic
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 64 - protein, 213 aa, synthetic construct
  REGION 1..213: note = Synthetic
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 451 aa, synthetic construct
  REGION 1..451: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 452 aa, synthetic construct
  REGION 1..452: note = Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 68 - protein, 214 aa, synthetic construct
  REGION 1..214: note = Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 449 aa, synthetic construct
  REGION 1..449: note = Synthetic
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 70 - protein, 214 aa, synthetic construct
  REGION 1..214: note = Synthetic
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - skipped

SEQ ID NO: 72 - protein, 184 aa, synthetic construct
  REGION 1..184: note = Synthetic
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 73 - protein, 364 aa, synthetic construct
  REGION 1..364: note = Synthetic
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 74 - protein, 829 aa, synthetic construct
  REGION 1..829: note = Synthetic
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 75 - protein, 1255 aa, synthetic construct [sequence omitted]

SEQ ID NO: 76 - protein, 300 aa, synthetic construct
  REGION 1..300: note = Synthetic
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 77 - protein, 243 aa, synthetic construct
  REGION 1..243: note = Synthetic
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 78 - protein, 229 aa, synthetic construct
  REGION 1..229: note = Synthetic
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 79 - protein, 534 aa, synthetic construct
  REGION 1..534: note = Synthetic
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 80 - protein, 117 aa, synthetic construct [sequence omitted]

SEQ ID NO: 81 - protein, 111 aa, synthetic construct [sequence omitted]

SEQ ID NO: 82 - protein, 10 aa, synthetic construct
  REGION 1..10: note = Synthetic
GYTFSSYWIE

SEQ ID NO: 83 - protein, 17 aa, synthetic construct
  REGION 1..17: note = Synthetic
EILPGGGDTNYNEIFKG

SEQ ID NO: 84 - protein, 8 aa, synthetic construct
  REGION 1..8: note = Synthetic
RVPIRLDY

SEQ ID NO: 85 - protein, 18 aa, synthetic construct
  REGION 1..18: note = Synthetic
ITCKASQSVDYEGDSFLN

SEQ ID NO: 86 - protein, 7 aa, synthetic construct
  REGION 1..7: note = Synthetic
AASNLES

SEQ ID NO: 87 - protein, 9 aa, synthetic construct
  REGION 1..9: note = Synthetic
QQSNEDPLT

SEQ ID NO: 88 - protein, 447 aa, synthetic construct
  REGION 1..447: note = Synthetic
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 89 - protein, 218 aa, synthetic construct
  REGION 1..218: note = Synthetic
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 116 aa, synthetic construct
  REGION 1..116: note = Synthetic
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 91 - protein, 109 aa, synthetic construct
  REGION 1..109: note = Synthetic
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVL

SEQ ID NO: 92 - protein, 449 aa, synthetic construct
  REGION 1..449: note = Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 93 - protein, 214 aa, synthetic construct
  REGION 1..214: note = Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 448 aa, synthetic construct
  REGION 1..448: note = Synthetic
QVQLVQSGAEVKKPGASVKMSCKASGYTFTSYWMHWVRQAPGQGLEWIGAIYPGNSETGYAQKFQGRATLTADTSTSTAYMELSSLRSEDTAVYYCTRENWDPGFAFWGQGTLITVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 95 - protein, 218 aa, synthetic construct
  REGION 1..218: note = Synthetic
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 446 aa, synthetic construct
  REGION 1..446: note = Synthetic
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 97 - protein, 219 aa, synthetic construct
  REGION 1..219: note = Synthetic
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 448 aa, synthetic construct
  REGION 1..448: note = Synthetic
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 99 - protein, 218 aa, synthetic construct; identical to SEQ ID NO: 95
  REGION 1..218: note = Synthetic

SEQ ID NO: 100 - protein, 453 aa, synthetic construct
  REGION 1..453: note = Synthetic
QVQLQESGPGLVKPSQTLSLTCTVSGGSISSGGYYWSWIRQHPGKGLEWIGIIYYSGSTYYNPSLKSRVTISVDTSKNQFSLKLNSVTAADTAVFYCARVAIVTTIPGGMDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 101 - protein, 214 aa, synthetic construct
  REGION 1..214: note = Synthetic
EIVLTQSPDFQSVTPKEKVTITCRASQSIGISLHWYQQKPDQSPKLLIKYASQSFSGVPSRFSGSGSGTDFTLTINSLEAEDAATYYCHQSRSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 448 aa, synthetic construct
  REGION 1..448: note = Synthetic
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 103 - protein, 218 aa, synthetic construct
  REGION 1..218: note = Synthetic
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 446 aa, synthetic construct
  REGION 1..446: note = Synthetic
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 105 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 450 aa, synthetic construct
  REGION 1..450: note = Synthetic
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 107 - protein, 219 aa, synthetic construct
  REGION 1..219: note = Synthetic
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 447 aa, synthetic construct
  REGION 1..447: note = Synthetic
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 109 - protein, 214 aa, synthetic construct
  REGION 1..214: note = Synthetic
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 451 aa, synthetic construct
  REGION 1..451: note = Synthetic
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 111 - protein, 214 aa, synthetic construct
  REGION 1..214: note = Synthetic
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 450 aa, synthetic construct
  REGION 1..450: note = Synthetic
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 113 - protein, 219 aa, synthetic construct
  REGION 1..219: note = Synthetic
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 449 aa, synthetic construct
  REGION 1..449: note = Synthetic
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 115 - protein, 215 aa, synthetic construct
  REGION 1..215: note = Synthetic
EIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 450 aa, synthetic construct
  REGION 1..450: note = Synthetic
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 117 - protein, 217 aa, synthetic construct
  REGION 1..217: note = Synthetic
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 118 - protein, 450 aa, synthetic construct
  REGION 1..450: note = Synthetic
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 119 - protein, 219 aa, synthetic construct
  REGION 1..219: note = Synthetic
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 448 aa, synthetic construct
  REGION 1..448: note = Synthetic
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 121 - protein, 214 aa, synthetic construct
  REGION 1..214: note = Synthetic
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 446 aa, synthetic construct
  REGION 1..446: note = Synthetic
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 123 - protein, 214 aa, synthetic construct
  REGION 1..214: note = Synthetic
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 446 aa, synthetic construct
  REGION 1..446: note = Synthetic
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 125 - protein, 214 aa, synthetic construct
  REGION 1..214: note = Synthetic
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 1210 aa, Homo sapiens [sequence omitted]

SEQ ID NO: 127 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 128 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 129 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 130 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 131 - protein, 875 aa, Homo sapiens [sequence omitted]

SEQ ID NO: 132 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 133 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 134 - protein, 690 aa, Homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSVLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL

SEQ ID NO: 135 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 136 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 137 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSEHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 138 - protein, 572 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 139 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 140 - protein, 296 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 141 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 142 - protein, 14507 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 143 - protein, 447 aa, synthetic construct
  REGION 1..447: note = Synthetic
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 144 - protein, 214 aa, synthetic construct
  REGION 1..214: note = Synthetic
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 4 aa, synthetic construct
  REGION 1..4: note = Synthetic
GFLG

SEQ ID NO: 146 - protein, 4 aa, synthetic construct
  REGION 1..4: note = Synthetic
GLFG

SEQ ID NO: 147 - protein, 4 aa, synthetic construct
  REGION 1..4: note = Synthetic
ALAL

SEQ ID NO: 148 - protein, 4 aa, synthetic construct
  REGION 1..4: note = Synthetic
GGGG

SEQ ID NO: 149 - protein, 4 aa, synthetic construct
  REGION 1..4: note = Synthetic
GFVG

SEQ ID NO: 150 - protein, 4 aa, synthetic construct
  REGION 1..4: note = Synthetic
GVFG

SEQ ID NO: 151 - protein, 116 aa, synthetic construct
  REGION 1..116: note = Synthetic
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 152 - protein, 107 aa, synthetic construct
  REGION 1..107: note = Synthetic
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 153 - protein, 120 aa, synthetic construct
  REGION 1..120: note = Synthetic
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 154 - protein, 112 aa, synthetic construct
  REGION 1..112: note = Synthetic
DIVMTQTPLSLPVTPGEPASISCRSSQSLVHRNQNTYLHWYLQKPGQSPQLLIYTVSNRFSGVPDRFSGSGSGTDFTLKISRVEAEDVGVYFCSQSSHVPPTFGQGTKLEIK

SEQ ID NO: 155 - protein, 121 aa, synthetic construct
  REGION 1..121: note = Synthetic
QVQLVQSGAEVKKPGASVKVSCKASGYTFTSYWINWVRQAPGQGLEWIGNIFPDTTTTNYNEKFKGRVTLTRDTSISTAYMELSRLRSDDTAVYYCAREYYDGTYDAMDYWGQGTLVTVSS

SEQ ID NO: 156 - protein, 107 aa, synthetic construct
  REGION 1..107: note = Synthetic
AIQMTQSPSSLSASVGDRVTITCKASQSVNNDVAWYQQKPGKAPKLLIYYASNRYTGVPSRFSGSGSGTDFTLTISSLQPEDFATYFCQQDYSSPRTFGQGTKLEIK

SEQ ID NO: 157 - protein, 118 aa, synthetic construct
  REGION 1..118: note = Synthetic
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 158 - protein, 112 aa, synthetic construct
  REGION 1..112: note = Synthetic
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 159 - protein, 118 aa, synthetic construct
  REGION 1..118: note = Synthetic
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 160 - protein, 107 aa, synthetic construct
  REGION 1..107: note = Synthetic
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 161 - protein, 117 aa, synthetic construct
  REGION 1..117: note = Synthetic
EVQLQQSGAELVKPGASVKLSCTASGFNIRDTYIHWVKQRPEQGLEWIGRIDPPNGNLKYDPKFQGKATITADTSSNTAYLQFSSLTSEDTAVYYCARSDGYSFAYWGQGTLVTVSS

SEQ ID NO: 162 - protein, 112 aa, synthetic construct
  REGION 1..112: note = Synthetic
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHVEYPFTFGSGTKLEIK

SEQ ID NO: 163 - protein, 117 aa, synthetic construct
  REGION 1..117: note = Synthetic
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 164 - protein, 112 aa, synthetic construct
  REGION 1..112: note = Synthetic
DIVMTQSPLSLPVTPGEPASISCRSSQSLVHSNRDTYLRWYLQKPGQSPQLLIYKVSNRFSGVPDRFSGSGSGTDFTLKISRVEAEDVGVYYCSQSTHVPYTFGGGTKVEIK

SEQ ID NO: 165 - protein, 122 aa, synthetic construct
  REGION 1..122: note = Synthetic
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 166 - protein, 106 aa, synthetic construct
  REGION 1..106: note = Synthetic
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 167 - protein, 128 aa, synthetic construct
  REGION 1..128: note = Synthetic
QVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARLDHRYHEDTVYPGMDVWGQGTLVTVSS

SEQ ID NO: 168 - protein, 107 aa, synthetic construct
  REGION 1..107: note = Synthetic
DIELTQPPSVSVAPGQTARISCSGDNLPAYTVTWYQQKPGQAPVLVIYDDSDRPSGIPERFSGSNSGNTATLTISGTQAEDEADYYCASWDPSSGVVFGGGTKLTVL

SEQ ID NO: 169 - protein, 10 aa, synthetic construct
  REGION 1..10: note = Synthetic
GYTFTNYGVN

SEQ ID NO: 170 - protein, 17 aa, synthetic construct
  REGION 1..17: note = Synthetic
WINPNTGEPTFDDDFKG

SEQ ID NO: 171 - protein, 11 aa, synthetic construct
  REGION 1..11: note = Synthetic
SRGKNEAWFAY

SEQ ID NO: 172 - protein, 16 aa, synthetic construct
  REGION 1..16: note = Synthetic
RSSQSLVHRNQNTYLH

SEQ ID NO: 173 - protein, 7 aa, synthetic construct
  REGION 1..7: note = Synthetic
TVSNRFS

SEQ ID NO: 174 - protein, 9 aa, synthetic construct
  REGION 1..9: note = Synthetic
SQSSHVPPT

SEQ ID NO: 175 - protein, 10 aa, synthetic construct
  REGION 1..10: note = Synthetic
GYTFTSYWIN

SEQ ID NO: 176 - protein, 17 aa, synthetic construct
  REGION 1..17: note = Synthetic
NIFPDTTTTNYNEKFKG

SEQ ID NO: 177 - protein, 12 aa, synthetic construct
  REGION 1..12: note = Synthetic
EYYDGTYDAMDY

SEQ ID NO: 178 - protein, 11 aa, synthetic construct
  REGION 1..11: note = Synthetic
KASQSVNNDVA

SEQ ID NO: 179 - protein, 7 aa, synthetic construct
  REGION 1..7: note = Synthetic
YASNRYT

SEQ ID NO: 180 - protein, 9 aa, synthetic construct
  REGION 1..9: note = Synthetic
QQDYSSPRT

SEQ ID NO: 181 - protein, 10 aa, synthetic construct
  REGION 1..10: note = Synthetic
GFTFSSFGMH

SEQ ID NO: 182 - protein, 17 aa, synthetic construct
  REGION 1..17: note = Synthetic
YISSGSFTIYYADSVKG

SEQ ID NO: 183 - protein, 9 aa, synthetic construct
  REGION 1..9: note = Synthetic
MRKGYAMDY

SEQ ID NO: 184 - protein, 16 aa, synthetic construct
  REGION 1..16: note = Synthetic
RSSQIIIHSDGNTYLE

SEQ ID NO: 185 - protein, 7 aa, synthetic construct
  REGION 1..7: note = Synthetic
KVSNRFS

SEQ ID NO: 186 - protein, 9 aa, synthetic construct
  REGION 1..9: note = Synthetic
FQGSHVPHT

SEQ ID NO: 187 - protein, 10 aa, synthetic construct
  REGION 1..10: note = Synthetic
GYTFTNYGMN

SEQ ID NO: 188 - protein, 17 aa, synthetic construct
  REGION 1..17: note = Synthetic
WINTYTGEPTYADDFKG

SEQ ID NO: 189 - protein, 9 aa, synthetic construct
  REGION 1..9: note = Synthetic
IGDSSPSDY

SEQ ID NO: 190 - protein, 11 aa, synthetic construct
  REGION 1..11: note = Synthetic
KASQSVSNDVV

SEQ ID NO: 191 - protein, 9 aa, synthetic construct
  REGION 1..9: note = Synthetic
QQDYTSPWT

SEQ ID NO: 192 - protein, 10 aa, synthetic construct
  REGION 1..10: note = Synthetic
GFNIRDTYIH

SEQ ID NO: 193 - protein, 17 aa, synthetic construct
  REGION 1..17: note = Synthetic
RIDPPNGNLKYDPKFQG

SEQ ID NO: 194 - protein, 8 aa, synthetic construct
  REGION 1..8: note = Synthetic
SDGYSFAY

SEQ ID NO: 195 - protein, 16 aa, synthetic construct
  REGION 1..16: note = Synthetic
RSSKSLLHSNGNTYLY

SEQ ID NO: 196 - protein, 7 aa, synthetic construct
  REGION 1..7: note = Synthetic
RMSNLAS

SEQ ID NO: 197 - protein, 9 aa, synthetic construct
  REGION 1..9: note = Synthetic
MQHVEYPFT

SEQ ID NO: 198 - protein, 10 aa, synthetic construct
  REGION 1..10: note = Synthetic
GYTFSSYWMH

SEQ ID NO: 199 - protein, 17 aa, synthetic construct
  REGION 1..17: note = Synthetic
LIHPESGSTNYNEMFKN

SEQ ID NO: 200 - protein, 8 aa, synthetic construct
  REGION 1..8: note = Synthetic
GGRLYFDY

SEQ ID NO: 201 - protein, 16 aa, synthetic construct
  REGION 1..16: note = Synthetic
RSSQSLVHSNRDTYLR

SEQ ID NO: 202 - protein, 9 aa, synthetic construct
  REGION 1..9: note = Synthetic
SQSTHVPYT

SEQ ID NO: 203 - protein, 10 aa, synthetic construct
  REGION 1..10: note = Synthetic
GYTFTNYVMH

SEQ ID NO: 204 - protein, 17 aa, synthetic construct
  REGION 1..17: note = Synthetic
YINPYNDDVKYNEKFKG

SEQ ID NO: 205 - protein, 13 aa, synthetic construct
  REGION 1..13: note = Synthetic
WGYYGSPLYYFDY

SEQ ID NO: 206 - protein, 10 aa, synthetic construct
  REGION 1..10: note = Synthetic
RASSRLIYMH

SEQ ID NO: 207 - protein, 7 aa, synthetic construct
  REGION 1..7: note = Synthetic
ATSNLAS

SEQ ID NO: 208 - protein, 9 aa, synthetic construct
  REGION 1..9: note = Synthetic
QQWNSNPPT

SEQ ID NO: 209 - protein, 12 aa, synthetic construct
  REGION 1..12: note = Synthetic
GDSVSSNSAAWS

SEQ ID NO: 210 - protein, 18 aa, synthetic construct
  REGION 1..18: note = Synthetic
RIYYRSKWYNDYAVSVKS

SEQ ID NO: 211 - protein, 16 aa, synthetic construct
  REGION 1..16: note = Synthetic
LDHRYHEDTVYPGMDV

SEQ ID NO: 212 - protein, 11 aa, synthetic construct
  REGION 1..11: note = Synthetic
SGDNLPAYTVT

SEQ ID NO: 213 - protein, 7 aa, synthetic construct
  REGION 1..7: note = Synthetic
DDSDRPS

SEQ ID NO: 214 - protein, 10 aa, synthetic construct
  REGION 1..10: note = Synthetic
ASWDPSSGVV

SEQ ID NO: 215 - protein, 11 aa, synthetic construct
  REGION 1..11: note = Synthetic
QSLVHRNGNTY

SEQ ID NO: 216 - protein, 219 aa, synthetic construct
  REGION 1..219: note = Synthetic
DIVMTQTPLSLPVTPGEPASISCRSSQSLVHRNQNTYLHWYLQKPGQSPQLLIYTVSNRFSGVPDRFSGSGSGTDFTLKISRVEAEDVGVYFCSQSSHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 451 aa, synthetic construct
  REGION 1..451: note = Synthetic
QVQLVQSGAEVKKPGASVKVSCKASGYTFTSYWINWVRQAPGQGLEWIGNIFPDTTTTNYNEKFKGRVTLTRDTSISTAYMELSRLRSDDTAVYYCAREYYDGTYDAMDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 218 - protein, 214 aa, synthetic construct
  REGION 1..214: note = Synthetic
AIQMTQSPSSLSASVGDRVTITCKASQSVNNDVAWYQQKPGKAPKLLIYYASNRYTGVPSRFSGSGSGTDFTLTISSLQPEDFATYFCQQDYSSPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 448 aa, synthetic construct
  REGION 1..448: note = Synthetic
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 220 - protein, 219 aa, synthetic construct
  REGION 1..219: note = Synthetic
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 448 aa, synthetic construct
  REGION 1..448: note = Synthetic
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 222 - protein, 214 aa, synthetic construct
  REGION 1..214: note = Synthetic
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 447 aa, synthetic construct
  REGION 1..447: note = Synthetic
EVQLQQSGAELVKPGASVKLSCTASGFNIRDTYIHWVKQRPEQGLEWIGRIDPPNGNLKYDPKFQGKATITADTSSNTAYLQFSSLTSEDTAVYYCARSDGYSFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 224 - protein, 219 aa, synthetic construct
  REGION 1..219: note = Synthetic
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHVEYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 447 aa, synthetic construct
  REGION 1..447: note = Synthetic
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 226 - protein, 219 aa, synthetic construct
  REGION 1..219: note = Synthetic
DIVMTQSPLSLPVTPGEPASISCRSSQSLVHSNRDTYLRWYLQKPGQSPQLLIYKVSNRFSGVPDRFSGSGSGTDFTLKISRVEAEDVGVYYCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 452 aa, synthetic construct
  REGION 1..452: note = Synthetic
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 228 - protein, 213 aa, synthetic construct
  REGION 1..213: note = Synthetic
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 457 aa, synthetic construct
  REGION 1..457: note = Synthetic
QVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARLDHRYHEDTVYPGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 230 - protein, 213 aa, synthetic construct
  REGION 1..213: note = Synthetic
DIELTQPPSVSVAPGQTARISCSGDNLPAYTVTWYQQKPGQAPVLVIYDDSDRPSGIPERFSGSNSGNTATLTISGTQAEDEADYYCASWDPSSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 231 - protein, 994 aa, synthetic construct
  REGION 1..994: note = Synthetic
MRPVALLLLPSLLALLAHGLSLEAPTVGKGQAPGIEETDGELTAAPTPEQPERGVHFVTTAPTLKLLNHHPLLEEFLQEGLEKGDEELRPALPFQPDPPAPFTPSPLPRLANQDSRPVFTSPTPAMAAVPTQPQSKEGPWSPESESPMLRITAPLPPGPSMAVPTLGPGEIASTTPPSRAWTPTQEGPGDMGRPWVAEVVSQGAGIGIQGTITSSTASGDDEETTTTTTIITTTITTVQTPGPCSWNFSGPEGSLDSPTDLSSPTDVGLDCFFYISVYPGYGVEIKVQNISLREGETVTVEGLGGPDPLPLANQSFLLRGQVIRSPTHQAALRFQSLPPPAGPGTFHFHYQAYLLSCHFPRRPAYGDVTVTSLHPGGSARFHCATGYQLKGARHLTCLNATQPFWDSKEPVCIAACGGVIRNATTGRIVSPGFPGNYSNNLTCHWLLEAPEGQRLHLHFEKVSLAEDDDRLIIRNGDNVEAPPVYDSYEVEYLPIEGLLSSGKHFFVELSTDSSGAAAGMALRYEAFQQGHCYEPFVKYGNFSSSTPTYPVGTTVEFSCDPGYTLEQGSIIIECVDPHDPQWNETEPACRAVCSGEITDSAGVVLSPNWPEPYGRGQDCIWGVHVEEDKRIMLDIRVLRIGPGDVLTFYDGDDLTARVLGQYSGPRSHFKLFTSMADVTIQFQSDPGTSVLGYQQGFVIHFFEVPRNDTCPELPEIPNGWKSPSQPELVHGTVVTYQCYPGYQVVGSSVLMCQWDLTWSEDLPSCQRVTSCHDPGDVEHSRRLISSPKFPVGATVQYICDQGFVLMGSSILTCHDRQAGSPKWSDRAPKCLLEQLKPCHGLSAPENGARSPEKQLHPAGATIHFSCAPGYVLKGQASIKCVPGHPSHWSDPPPICRAASLDGFYNSRSLDVAKAPAASSTLDAAHIAAAIFLPLVAMVLLVGGVYFYFSRLQGKSSLQLPRPRPRPYNRITIESAFDNPTYETGSLSFAGDERI

SEQ ID NO: 232 - protein, 858 aa, synthetic construct
  REGION 1..858: note = Synthetic
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQETLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 233 - protein, 618 aa, synthetic construct
  REGION 1..618: note = Synthetic
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 234 - protein, 383 aa, synthetic construct
  REGION 1..383: note = Synthetic
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNRTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 235 - DNA, 1350 bp, synthetic construct
  misc_feature 1..1350: note = Synthetic
  source 1..1350: mol_type = other DNA
caggttcagctccagcagtctggcagcgagctgaaaaaacctggcgcctccgtgaaggtgtcctgcaaggcttctggctacacctttaccaactacggcgtgaactggatcaagcaggcccctggacaaggcctccaatggatgggctggatcaaccccaataccggcgagcccaccttcgacgacgatttcaagggcagattcgccttcagcctggacacctctgtgtccacagcctacctccagatcagcagcctgaaggccgatgataccgccgtgtacttctgctccagaagccggggaaagaacgaggcttggtttgcctattggggccagggcacactggtcaccgttagctctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 236 - protein, 450 aa, synthetic construct
  REGION 1..450: note = Synthetic
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 237 - protein, 219 aa, synthetic construct; identical to SEQ ID NO: 119
  REGION 1..219: note = Synthetic

SEQ ID NO: 238 - DNA, 657 bp, synthetic construct
  misc_feature 1..657: note = Synthetic
  source 1..657: mol_type = other DNA
gacattcagctgacacagagccctctgagcctgcctgttacactgggacagcctgccagcatcagctgtagaagcagccagagcctggtgcacagaaacggcaacacctacctgcactggttccagcagaggcctggccagtctcctagactgctgatctacaccgtgtccaacagattcagcggcgtgcccgatagattttccggcagcggctctggcaccgacttcaccctgaagattagcagagtggaagccgaggacgtgggcgtgtacttctgtagccagtctagccacgtgccacctacctttggcgccggaaccagactggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 239 - protein, 217 aa, synthetic construct
  REGION 1..217: note = Synthetic
DIVMTQTPLSLPVTPGEPASISCRSSQSLVHRNQNTYLHWYLQKPGQSPQLLIYTVSNRFSGVPDRFSGSGSGTDFTLKSRVEAEDVGVYFCSQSSHVPPTFGQGTKLEIKRTVAAPSVFIFPPSDEQLKSGTASVVCLLNNFYPREAKVQWKVDNALSGNSQESVTEQDSKDSTYSLSSTLTLSKADYEKHKVYACEVTHQGLSSPVTKSFNRGEC

SEQ ID NO: 240 - protein, 451 aa, synthetic construct
  REGION 1..451: note = Synthetic
EVQLLESGGGLVQPGGSLRLSCAASGFTFSSFAMSWVRQAPGKGLEWVSAISGFGGSTYYADSVKGRFTISRDNSKNTLYLQMNSLRAEDTAVYYCARQFWEDQPFYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 241 - DNA, 1353 bp, synthetic construct
  misc_feature 1..1353: note = Synthetic
  source 1..1353: mol_type = other DNA
gaggtgcagctgctggagagcggcggcggcctggtgcagcccggcggcagcctgcggctgagctgcgccgccagcggcttcaccttcagcagcttcgccatgagctgggtgcggcaggcccccggcaagggcctggagtgggtgagcgccatcagcggcttcggcggcagcacctactacgccgacagcgtgaagggccggttcaccatcagccgggacaacagcaagaacaccctgtacctgcagatgaacagcctgcgggccgaggacaccgccgtgtactactgcgcccggcagttctgggaggaccagcccttctacttcgactactggggccagggcaccctggtgaccgtgagcagcgccagcaccaagggccccagcgtgttccccctggcccccagcagcaagagcaccagcggcggcaccgccgccctgggctgcctggtgaaggactacttcccctgccccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctacccctgcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 242 - protein, 451 aa, synthetic construct
  REGION 1..451: note = Synthetic
EVQLLESGGGLVQPGGSLRLSCAASGFTFSSFAMSWVRQAPGKGLEWVSAISGFGGSTYYADSVKGRFTISRDNSKNTLYLQMNSLRAEDTAVYYCARQFWEDQPFYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 243 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - DNA, 642 bp, synthetic construct
  misc_feature 1..642: note = Synthetic
  source 1..642: mol_type = other DNA
gacatccagatgacccagagccccagcagcctgagcgccagcgtgggcgaccgggtgaccatcacctgccgggccagccagagcatcagcagctacctgaactggtaccagcagaagcccggcaaggcccccaagctgctgatctacgccgccagcagcctgcagagcggcgtgcccagccggttcagcggcagcggcagcggcaccgacttcaccctgaccatcagcagcctgcagcccgaggacttcgccacctactactgccagcagagctacagcacccccctgaccttcggccagggcaccaaggtggagatcaagcggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgc

SEQ ID NO: 245 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 246 - DNA, 1350 bp, synthetic construct
  misc_feature 1..1350: note = Synthetic
  source 1..1350: mol_type = other DNA
gaggtgcagctggtggagagcggcggcggcctggtgcagcccggcggcagcctgcggctgagctgcgccgccagcggcttcaacatcaaggacacctacatccactgggtgcggcaggcccccggcaagggcctggagtgggtggcccggatctaccccaccaacggctacacccggtacgccgacagcgtgaagggccggttcaccatcagcgccgacaccagcaagaacaccgcctacctgcagatgaacagcctgcgggccgaggacaccgccgtgtactactgcagccggtggggcggcgacggcttctacgccatggactactggggccagggcaccctggtgaccgtgagcagcgccagcaccaagggccccagcgtgttccccctggcccccagcagcaagagcaccagcggcggcaccgccgccctgggctgcctggtgaaggactacttcccctgccccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctacccctgcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 247 - DNA, 642 bp, synthetic construct
  misc_feature 1..642: note = Synthetic
  source 1..642: mol_type = other DNA
gacatccagatgacccagagccccagcagcctgagcgccagcgtgggcgaccgggtgaccatcacctgccgggccagccaggacgtgaacaccgccgtggcctggtaccagcagaagcccggcaaggcccccaagctgctgatctacagcgccagcttcctgtacagcggcgtgcccagccggttcagcggcagccggagcggcaccgacttcaccctgaccatcagcagcctgcagcccgaggacttcgccacctactactgccagcagcactacaccaccccccccaccttcggccagggcaccaaggtggagatcaagcggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgc

SEQ ID NO: 248 - protein, 448 aa, synthetic construct
  REGION 1..448: note = Synthetic
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - DNA, 1344 bp, synthetic construct
  misc_feature 1..1344: note = Synthetic
  source 1..1344: mol_type = other DNA
gaggtgcagctggtgcagagcggcgccgaggtgaagaagcccggcgagagcctgaagatcagctgcaaggtgagcggctacaccttcaccgaccacaccatccactggatgcggcagatgcccggcaagggcctggagtggatgggctacatctacccccggagcggcagcatcaactacaacgagaagttcaagggccaggtgaccatcagcgccgacaagagcagcagcaccgcctacctgcagtggagcagcctgaaggccagcgacaccgccatgtactactgcgcccggcggaacctgttcctgcccatggagtactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagcgggtggagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 250 - protein, 220 aa, synthetic construct
  REGION 1..220: note = Synthetic
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - DNA, 660 bp, synthetic construct
  misc_feature 1..660: note = Synthetic
  source 1..660: mol_type = other DNA
gacatcgtgatgacccagacccccctgagcctgcccgtgacccccggcgagcccgccagcatcagctgccggagcagccagagcctgctgagcagcggcgaccagaagaactacctgacctggtacctgcagaagcccggccagagcccccagctgctgatctactgggccagcacccgggagagcggcgtgcccgaccggttcagcggcagcggcagcggcaccgacttcaccctgaagatcagccgggtggaggccgaggacgtgggcgtgtactactgccagaacgactaccggtaccccctgaccttcggccagggcaccaagctggagatcaagcggaccgtggccgcccccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaaccggggcgagtgc

SEQ ID NO: 252 - protein, 458 aa, synthetic construct
  REGION 1..458: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 253 - DNA, 1374 bp, synthetic construct
  misc_feature 1..1374: note = Synthetic
  source 1..1374: mol_type = other DNA
gaagttcagctgcttgaatctggcggcggactggttcaacctggcggatctctgagactgagctgtgccgccagcggcttcaccttcagccactatatgatggcctgggtccgacaggcccctggcaaaggacttgaatgggtgtccagaatcggcccctctggcggccctacacactacgctgattctgtgaagggcagattcaccatcagccgggacaacagcaagaacaccctgtacctgcagatgaacagcctgagagccgaggacaccgccgtgtattactgtgccggctacgacagcggctacgattatgtggctgtggccggacctgccgagtactttcagcattggggacagggcaccctggtcaccgttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 254 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - DNA, 642 bp, synthetic construct
  misc_feature 1..642: note = Synthetic
  source 1..642: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaacagctacagccggacctttggccagggaacaaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 256 - protein, 7 aa, synthetic construct
  REGION 1..7: note = Synthetic
GYTFTNY

SEQ ID NO: 257 - protein, 6 aa, synthetic construct
  REGION 1..6: note = Synthetic
NPNTGE

SEQ ID NO: 258 - protein, 5 aa, synthetic construct
  REGION 1..5: note = Synthetic
NYGVN

SEQ ID NO: 259 - protein, 8 aa, synthetic construct
  REGION 1..8: note = Synthetic
GYTFTNYG

SEQ ID NO: 260 - protein, 8 aa, synthetic construct
  REGION 1..8: note = Synthetic
INPNTGEP

SEQ ID NO: 261 - protein, 13 aa, synthetic construct
  REGION 1..13: note = Synthetic
SRSRGKNEAWFAY

SEQ ID NO: 262 - protein, 112 aa, synthetic construct
  REGION 1..112: note = Synthetic
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 263 - protein, 12 aa, synthetic construct
  REGION 1..12: note = Synthetic
SQSLVHRNGNTY

SEQ ID NO: 264 - skipped

SEQ ID NO: 265 - protein, 6 aa, synthetic construct
  REGION 1..6: note = Synthetic
SSHVPP

SEQ ID NO: 266 - protein, 16 aa, synthetic construct
  REGION 1..16: note = Synthetic
RSSQSLVHRNGNTYLH

SEQ ID NO: 267 - protein, 111 aa, synthetic construct
  REGION 1..111: note = Synthetic
DIVMTQTPLSLPVTPGEPASISCRSSQSLVHRNQNTYLHWYLQKPGQSPQLLIYTVSNRFSGVPDRFSGSGSGTDFTLKSRVEAEDVGVYFCSQSSHVPPTFGQGTKLEIK

SEQ ID NO: 268 - protein, 12 aa, synthetic construct
  REGION 1..12: note = Synthetic
SQSLVHRNQNTY

SEQ ID NO: 269 - protein, 11 aa, synthetic construct
  REGION 1..11: note = Synthetic
QSLVHRNQNTY

SEQ ID NO: 270 - protein, 121 aa, synthetic construct
  REGION 1..121: note = Synthetic
EVQLLESGGGLVQPGGSLRLSCAASGFTFSSFAMSWVRQAPGKGLEWVSAISGFGGSTYYADSVKGRFTISRDNSKNTLYLQMNSLRAEDTAVYYCARQFWEDQPFYFDYWGQGTLVTVSS

SEQ ID NO: 271 - protein, 7 aa, synthetic construct
  REGION 1..7: note = Synthetic
GFTFSSF

SEQ ID NO: 272 - protein, 6 aa, synthetic construct
  REGION 1..6: note = Synthetic
SGFGGS

SEQ ID NO: 273 - protein, 12 aa, synthetic construct
  REGION 1..12: note = Synthetic
QFWEDQPFYFDY

SEQ ID NO: 274 - protein, 5 aa, synthetic construct
  REGION 1..5: note = Synthetic
SFAMS

SEQ ID NO: 275 - protein, 17 aa, synthetic construct
  REGION 1..17: note = Synthetic
AISGFGGSTYYADSVKG

SEQ ID NO: 276 - protein, 8 aa, synthetic construct
  REGION 1..8: note = Synthetic
GFTFSSFA

SEQ ID NO: 277 - protein, 8 aa, synthetic construct
  REGION 1..8: note = Synthetic
ISGFGGST

SEQ ID NO: 278 - protein, 14 aa, synthetic construct
  REGION 1..14: note = Synthetic
ARQFWEDQPFYFDY

SEQ ID NO: 279 - protein, 10 aa, synthetic construct
  REGION 1..10: note = Synthetic
GFTFSSFAMS

SEQ ID NO: 280 - protein, 107 aa, synthetic construct [sequence omitted]

SEQ ID NO: 281 - protein, 7 aa, synthetic construct
  REGION 1..7: note = Synthetic
SQSISSY

SEQ ID NO: 282 - skipped

SEQ ID NO: 283 - protein, 6 aa, synthetic construct
  REGION 1..6: note = Synthetic
SYSTPL

SEQ ID NO: 284 - protein, 11 aa, synthetic construct
  REGION 1..11: note = Synthetic
RASQSISSYLN

SEQ ID NO: 285 - protein, 7 aa, synthetic construct
  REGION 1..7: note = Synthetic
AASSLQS

SEQ ID NO: 286 - protein, 9 aa, synthetic construct
  REGION 1..9: note = Synthetic
QQSYSTPLT

SEQ ID NO: 287 - protein, 6 aa, synthetic construct
  REGION 1..6: note = Synthetic
QSISSY

SEQ ID NO: 288 - protein, 7 aa, synthetic construct
  REGION 1..7: note = Synthetic
AASSLQS

SEQ ID NO: 289 - protein, 7 aa, synthetic construct
  REGION 1..7: note = Synthetic
GFNIKDT

SEQ ID NO: 290 - protein, 6 aa, synthetic construct
  REGION 1..6: note = Synthetic
YPTNGY

SEQ ID NO: 291 - protein, 11 aa, synthetic construct
  REGION 1..11: note = Synthetic
WGGDGFYAMDY

SEQ ID NO: 292 - protein, 5 aa, synthetic construct
  REGION 1..5: note = Synthetic
DTYIH

SEQ ID NO: 293 - protein, 8 aa, synthetic construct
  REGION 1..8: note = Synthetic
GFNIKDTY

SEQ ID NO: 294 - protein, 8 aa, synthetic construct
  REGION 1..8: note = Synthetic
IYPTNGYT

SEQ ID NO: 295 - protein, 13 aa, synthetic construct
  REGION 1..13: note = Synthetic
SRWGGDGFYAMDY

SEQ ID NO: 296 - protein, 107 aa, synthetic construct [sequence omitted]

SEQ ID NO: 297 - protein, 7 aa, synthetic construct
  REGION 1..7: note = Synthetic
SQDVNTA

SEQ ID NO: 298 - skipped

SEQ ID NO: 299 - protein, 6 aa, synthetic construct
  REGION 1..6: note = Synthetic
HYTTPP

SEQ ID NO: 300 - protein, 11 aa, synthetic construct
  REGION 1..11: note = Synthetic
RASQDVNTAVA

SEQ ID NO: 301 - protein, 7 aa, synthetic construct
  REGION 1..7: note = Synthetic
SASFLYS

SEQ ID NO: 302 - protein, 6 aa, synthetic construct
  REGION 1..6: note = Synthetic
QDVNTA

SEQ ID NO: 303 - protein, 118 aa, synthetic construct
  REGION 1..118: note = Synthetic
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSS

SEQ ID NO: 304 - protein, 7 aa, synthetic construct
  REGION 1..7: note = Synthetic
GYTFTDH

SEQ ID NO: 305 - protein, 6 aa, synthetic construct
  REGION 1..6: note = Synthetic
YPRSGS

SEQ ID NO: 306 - protein, 9 aa, synthetic construct
  REGION 1..9: note = Synthetic
RNLFLPMEY

SEQ ID NO: 307 - protein, 5 aa, synthetic construct
  REGION 1..5: note = Synthetic
DHTIH

SEQ ID NO: 308 - protein, 17 aa, synthetic construct
  REGION 1..17: note = Synthetic
YIYPRSGSINYNEKFKG

SEQ ID NO: 309 - protein, 8 aa, synthetic construct
  REGION 1..8: note = Synthetic
GYTFTDHT

SEQ ID NO: 310 - protein, 10 aa, synthetic construct
  REGION 1..10: note = Synthetic
GYTFTDHTIH

SEQ ID NO: 311 - protein, 113 aa, synthetic construct
  REGION 1..113: note = Synthetic
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIK

SEQ ID NO: 312 - protein, 13 aa, synthetic construct
  REGION 1..13: note = Synthetic
SQSLLSSGDQKNY

SEQ ID NO: 313 - skipped

SEQ ID NO: 314 - protein, 6 aa, synthetic construct
  REGION 1..6: note = Synthetic
DYRYPL

SEQ ID NO: 315 - protein, 17 aa, synthetic construct
  REGION 1..17: note = Synthetic
RSSQSLLSSGDQKNYLT

SEQ ID NO: 316 - protein, 9 aa, synthetic construct
  REGION 1..9: note = Synthetic
QNDYRYPLT

SEQ ID NO: 317 - protein, 12 aa, synthetic construct
  REGION 1..12: note = Synthetic
QSLLSSGDQKNY

SEQ ID NO: 318 - protein, 128 aa, synthetic construct
  REGION 1..128: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 319 - protein, 7 aa, synthetic construct
  REGION 1..7: note = Synthetic
GFTFSHY

SEQ ID NO: 320 - protein, 6 aa, synthetic construct
  REGION 1..6: note = Synthetic
GPSGGP

SEQ ID NO: 321 - protein, 19 aa, synthetic construct
  REGION 1..19: note = Synthetic
YDSGYDYVAVAGPAEYFQH

SEQ ID NO: 322 - protein, 5 aa, synthetic construct
  REGION 1..5: note = Synthetic
HYMMA

SEQ ID NO: 323 - protein, 17 aa, synthetic construct
  REGION 1..17: note = Synthetic
RIGPSGGPTHYADSVKG

SEQ ID NO: 324 - protein, 19 aa, synthetic construct
  REGION 1..19: note = Synthetic
YDSGYDYVAVAGPAEYFQH

SEQ ID NO: 325 - protein, 8 aa, synthetic construct
  REGION 1..8: note = Synthetic
GFTFSHYM

SEQ ID NO: 326 - protein, 8 aa, synthetic construct
  REGION 1..8: note = Synthetic
IGPSGGPT

SEQ ID NO: 327 - protein, 21 aa, synthetic construct
  REGION 1..21: note = Synthetic
AGYDSGYDYVAVAGPAEYFQH

SEQ ID NO: 328 - protein, 10 aa, synthetic construct
  REGION 1..10: note = Synthetic
GFTFSHYMMA

SEQ ID NO: 329 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 330 - protein, 7 aa, synthetic construct
  REGION 1..7: note = Synthetic
SQSISTW

SEQ ID NO: 331 - skipped

SEQ ID NO: 332 - protein, 6 aa, synthetic construct
  REGION 1..6: note = Synthetic
YNSYSR

SEQ ID NO: 333 - protein, 11 aa, synthetic construct
  REGION 1..11: note = Synthetic
RASQSISTWLA

SEQ ID NO: 334 - protein, 7 aa, synthetic construct
  REGION 1..7: note = Synthetic
KASNLHT

SEQ ID NO: 335 - protein, 9 aa, synthetic construct
  REGION 1..9: note = Synthetic
QQYNSYSRT

SEQ ID NO: 336 - protein, 6 aa, synthetic construct
  REGION 1..6: note = Synthetic
QSISTW

SEQ ID NO: 337 - protein, 976 aa, synthetic construct
  REGION 1..976: note = Synthetic
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 338 - protein, 115 aa, synthetic construct
  REGION 1..115: note = Synthetic
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 339 - protein, 105 aa, synthetic construct
  REGION 1..105: note = Synthetic
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 340 - DNA, 345 bp, synthetic construct
  misc_feature 1..345: note = Synthetic
  source 1..345: mol_type = other DNA
gaagttcagctggttcagtctggcgccgaagtgaagaaacctggcgccaccgtgaagatcagctgcaaggtgtccggctacaccttcaccgactactacatccactgggtgcagcaggcccctggcaaaggacttgagtggatgggcagagtgaaccccgatcacggcgacagctactacaaccagaagttcaaggacaaggccaccatcaccgccgacaagagcaccgataccgcctacatggaactgagcagcctgagaagcgaggataccgccgtgtacttctgcgcccggaactacctgtttgaccactggggacagggcaccctggtcacagttagttct

SEQ ID NO: 341 - DNA, 315 bp, synthetic construct
  misc_feature 1..315: note = Synthetic
  source 1..315: mol_type = other DNA
gacattcagatgacacagagccctagcagcgtgtccgcctctgtgggagacagagtgaccatcacatgcaaggccagccaggatgtgggaacagccgtggcttggtatcagcagaagcctggcaaggcccctaagctgctgatctactgggccagcatcagacacacaggcgtgcccagcagattttctggcagcggctctggcaccgacttcaccctgaccatatctagcctgcagccagaggacttcgccacctactactgccaccagtttgccacctacaccttcggcggaggcaccaaggtggaaatcaag

SEQ ID NO: 342 - protein, 445 aa, synthetic construct
  REGION 1..445: note = Synthetic
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 343 - DNA, 1335 bp, synthetic construct
  misc_feature 1..1335: note = Synthetic
  source 1..1335: mol_type = other DNA
gaagttcagctggttcagtctggcgccgaagtgaagaaacctggcgccaccgtgaagatcagctgcaaggtgtccggctacaccttcaccgactactacatccactgggtgcagcaggcccctggcaaaggacttgagtggatgggcagagtgaaccccgatcacggcgacagctactacaaccagaagttcaaggacaaggccaccatcaccgccgacaagagcaccgataccgcctacatggaactgagcagcctgagaagcgaggataccgccgtgtacttctgcgcccggaactacctgtttgaccactggggacagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 344 - protein, 212 aa, synthetic construct
  REGION 1..212: note = Synthetic
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - DNA, 636 bp, synthetic construct
  misc_feature 1..636: note = Synthetic
  source 1..636: mol_type = other DNA
gacattcagatgacacagagccctagcagcgtgtccgcctctgtgggagacagagtgaccatcacatgcaaggccagccaggatgtgggaacagccgtggcttggtatcagcagaagcctggcaaggcccctaagctgctgatctactgggccagcatcagacacacaggcgtgcccagcagattttctggcagcggctctggcaccgacttcaccctgaccatatctagcctgcagccagaggacttcgccacctactactgccaccagtttgccacctacaccttcggcggaggcaccaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 346 - protein, 449 aa, synthetic construct
  REGION 1..449: note = Synthetic
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 347 - DNA, 1347 bp, synthetic construct
  misc_feature 1..1347: note = Synthetic
  source 1..1347: mol_type = other DNA
gaagtgcagctggttgaatctggcggaggactggttcagcctggcggatctctgagactgtcttgtgccgccagcggctacaccttcaccaacttcggcatgaactgggtccgacaggcccctggcaaaggccttgaatgggtcgcctggatcaacaccaataccggcgagcccagatacgccgaagagttcaagggcagattcaccatcagccgggacaacgccaagaacagcctgtacctgcagatgaactccctgagagccgaggacaccgccgtgtactactgtgccagagattgggacggcgcctacttcttcgattactggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 348 - protein, 449 aa, synthetic construct
  REGION 1..449: note = Synthetic
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 349 - DNA, 1347 bp, synthetic construct
  misc_feature 1..1347: note = Synthetic
  source 1..1347: mol_type = other DNA
gaagtgcagctggttgaatctggcggaggactggttcagcctggcggatctctgagactgtcttgtgccgccagcggctacaccttcaccaacttcggcatgaactgggtccgacaggcccctggcaaaggccttgaatgggtcgcctggatcaacaccaataccggcgagcccagatacgccgaagagttcaagggcagattcaccatcagccgggacaacgccaagaacagcctgtacctgcagatgaactccctgagagccgaggacaccgccgtgtactactgtgccagagattgggacggcgcctacttcttcgattactggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 350 - protein, 214 aa, synthetic construct
  REGION 1..214: note = Synthetic
DIQMTQSPSSLSASVGDRVTITCKASQSVSNDVAWYQQKPGKAPKLLIYFATNRYTGVPSRFSGSGYGTDFTLTISSLQPEDFATYYCQQDYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - DNA, 642 bp, synthetic construct
  misc_feature 1..642: note = Synthetic
  source 1..642: mol_type = other DNA
gacattcagatgacacagagccctagcagcctgagcgcctctgtgggagacagagtgaccatcacatgcaaggccagccagagcgtgtccaacgacgtggcatggtatcagcagaagcctggcaaggcccctaagctgctgatctacttcgccaccaacagatacaccggcgtgcccagcagattttccggctctggctacggcaccgacttcaccctgaccatatctagcctgcagccagaggatttcgccacctactactgccagcaggactacagcagcccttggacatttggccagggaacaaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 352 - protein, 451 aa, synthetic construct
  REGION 1..451: note = Synthetic
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 353 - DNA, 1353 bp, synthetic construct
  misc_feature 1..1353: note = Synthetic
  source 1..1353: mol_type = other DNA
caggttcagctggttcagtctggcgccgaagtgaagaaacctggcgcctctgtgaaggtgtcctgcaaggccagcggctacacctttacaacagccggcatgcagtgggtccgacaggctcctggacaaggcttggaatggatgggctggatcaacacccacagcggcgtgcccaaatacgccgaggatttcaagggcagagtgaccatcagcgccgacaccagcacaagcacagcctacctgcagctgagcagcctgaagtctgaggacaccgccgtgtactactgtgccagaagcggctttggcagcagctactggtacttcgatgtgtggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 354 - protein, 451 aa, synthetic construct
  REGION 1..451: note = Synthetic
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 355 - DNA, 1353 bp, synthetic construct
  misc_feature 1..1353: note = Synthetic
  source 1..1353: mol_type = other DNA
caggttcagctggttcagtctggcgccgaagtgaagaaacctggcgcctctgtgaaggtgtcctgcaaggccagcggctacacctttacaacagccggcatgcagtgggtccgacaggctcctggacaaggcttggaatggatgggctggatcaacacccacagcggcgtgcccaaatacgccgaggatttcaagggcagagtgaccatcagcgccgacaccagcacaagcacagcctacctgcagctgagcagcctgaagtctgaggacaccgccgtgtactactgtgccagaagcggctttggcagcagctactggtacttcgatgtgtggggccagggcaccctggtcacagttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 356 - protein, 214 aa, synthetic construct
  REGION 1..214: note = Synthetic
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - DNA, 645 bp, synthetic construct
  misc_feature 1..645: note = Synthetic
  source 1..645: mol_type = other DNA
gacattcagatgacacagagccctagcagcctgagcgcctctgtgggagacagagtgaccatcacatgcaaggccagccaggatgtgtctacagccgtggcctggtatcagcagaagcctggaaaggcccctaagctgctgatctacagcgccagctacagatacaccggcgtgcccagcagattttctggcagcggctctggcaccgacttcaccctgaccatatctagcctgcagccagaggacttcgccgtgtactactgccagcagcactacatcacccctctgacctttggccaggggaccaagctggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgctga

SEQ ID NO: 358 - protein, 10 aa, synthetic construct
  REGION 1..10: note = Synthetic
GYTFTDYYIH

SEQ ID NO: 359 - protein, 17 aa, synthetic construct
  REGION 1..17: note = Synthetic
RVNPDHGDSYYNQKFKD

SEQ ID NO: 360 - protein, 6 aa, synthetic construct
  REGION 1..6: note = Synthetic
NYLFDH

SEQ ID NO: 361 - protein, 5 aa, synthetic construct
  REGION 1..5: note = Synthetic
DYYIH

SEQ ID NO: 362 - protein, 7 aa, synthetic construct
  REGION 1..7: note = Synthetic
GYTFTDY

SEQ ID NO: 363 - protein, 6 aa, synthetic construct
  REGION 1..6: note = Synthetic
NPDHGD

SEQ ID NO: 364 - protein, 8 aa, synthetic construct
  REGION 1..8: note = Synthetic
GYTFTDYY

SEQ ID NO: 365 - protein, 8 aa, synthetic construct
  REGION 1..8: note = Synthetic
VNPDHGDS

SEQ ID NO: 366 - protein, 8 aa, synthetic construct
  REGION 1..8: note = Synthetic
ARNYLFDH

SEQ ID NO: 367 - protein, 11 aa, synthetic construct
  REGION 1..11: note = Synthetic
KASQDVGTAVA

SEQ ID NO: 368 - protein, 7 aa, synthetic construct
  REGION 1..7: note = Synthetic
WASIRHT

SEQ ID NO: 369 - protein, 7 aa, synthetic construct
  REGION 1..7: note = Synthetic
HQFATYT

SEQ ID NO: 370 - protein, 7 aa, synthetic construct
  REGION 1..7: note = Synthetic
SQDVGTA

SEQ ID NO: 371 - protein, 4 aa, synthetic construct
  REGION 1..4: note = Synthetic
FATY

SEQ ID NO: 372 - protein, 6 aa, synthetic construct
  REGION 1..6: note = Synthetic
QDVGTA

SEQ ID NO: 373 - protein, 10 aa, synthetic construct
  REGION 1..10: note = Synthetic
GYTFTNFGMN

SEQ ID NO: 374 - protein, 17 aa, synthetic construct
  REGION 1..17: note = Synthetic
WINTNTGEPRYAEEFKG

SEQ ID NO: 375 - protein, 10 aa, synthetic construct
  REGION 1..10: note = Synthetic
DWDGAYFFDY

SEQ ID NO: 376 - protein, 7 aa, synthetic construct
  REGION 1..7: note = Synthetic
GYTFTNF

SEQ ID NO: 377 - protein, 6 aa, synthetic construct
  REGION 1..6: note = Synthetic
NTNTGE

SEQ ID NO: 378 - protein, 5 aa, synthetic construct
  REGION 1..5: note = Synthetic
NFGMN

SEQ ID NO: 379 - protein, 8 aa, synthetic construct
  REGION 1..8: note = Synthetic
INTNTGEP

SEQ ID NO: 380 - protein, 12 aa, synthetic construct
  REGION 1..12: note = Synthetic
ARDWDGAYFFDY

SEQ ID NO: 381 - protein, 11 aa, synthetic construct
  REGION 1..11: note = Synthetic
KASQSVSNDVA

SEQ ID NO: 382 - protein, 7 aa, synthetic construct
  REGION 1..7: note = Synthetic
FATNRYT

SEQ ID NO: 383 - protein, 9 aa, synthetic construct
  REGION 1..9: note = Synthetic
QQDYSSPWT

SEQ ID NO: 384 - protein, 7 aa, synthetic construct
  REGION 1..7: note = Synthetic
SQSVSND

SEQ ID NO: 385 - skipped

SEQ ID NO: 386 - protein, 6 aa, synthetic construct
  REGION 1..6: note = Synthetic
DYSSPW

SEQ ID NO: 387 - protein, 6 aa, synthetic construct
  REGION 1..6: note = Synthetic
QSVSND

SEQ ID NO: 388 - protein, 10 aa, synthetic construct
  REGION 1..10: note = Synthetic
GYTFTTAGMQ

SEQ ID NO: 389 - protein, 17 aa, synthetic construct
  REGION 1..17: note = Synthetic
WINTHSGVPKYAEDFKG

SEQ ID NO: 390 - protein, 12 aa, synthetic construct
  REGION 1..12: note = Synthetic
SGFGSSYWYFDV

SEQ ID NO: 391 - protein, 8 aa, synthetic construct
  REGION 1..7: note = Synthetic
GYTFTTAG

SEQ ID NO: 392 - protein, 6 aa, synthetic construct
  REGION 1..6: note = Synthetic
NTHSGV

SEQ ID NO: 393 - protein, 5 aa, synthetic construct
  REGION 1..5: note = Synthetic
TAGMQ

SEQ ID NO: 394 - protein, 8 aa, synthetic construct
  REGION 1..8: note = Synthetic
INTHSGVP

SEQ ID NO: 395 - protein, 14 aa, synthetic construct
  REGION 1..14: note = Synthetic
ARSGFGSSYWYFDV

SEQ ID NO: 396 - protein, 11 aa, synthetic construct
  REGION 1..11: note = Synthetic
KASQDVSTAVA

SEQ ID NO: 397 - protein, 7 aa, synthetic construct
  REGION 1..7: note = Synthetic
SASYRYT

SEQ ID NO: 398 - protein, 9 aa, synthetic construct
  REGION 1..9: note = Synthetic
QQHYITPLT

SEQ ID NO: 399 - protein, 7 aa, synthetic construct
  REGION 1..7: note = Synthetic
SQDVSTA

SEQ ID NO: 400 - protein, 6 aa, synthetic construct
  REGION 1..6: note = Synthetic
HYITPL

SEQ ID NO: 401 - protein, 6 aa, synthetic construct
  REGION 1..6: note = Synthetic
QDVSTA

SEQ ID NO: 402 - protein, 7 aa, synthetic construct
  REGION 1..7: note = Synthetic
GYTFTTA